(19) United States
(12) Patent Application Publication
(10) Pub. No.: US 2022/0016243 A1
(43) Pub. Date: Jan. 20, 2022

(54) METHODS FOR TREATMENT OF CANCER WITH AN ANTI-TIGIT ANTAGONIST ANTIBODY

(71) Applicants: Genentech, Inc., South San Francisco, CA (US); Hoffmann-La Roche Inc., Little Falls, NJ (US)

(72) Inventors: Catherine LAI, South San Francisco, CA (US); Janet LAU, San Francisco, CA (US); Anthony Jongha LEE, San Mateo, CA (US); Shi LI, South San Francisco, CA (US); Yvonne Gail LIN-LIU, San Francisco, CA (US); Christina Jeanne MATHENY, San Francisco, CA (US); Diana MENDUS, San Francisco, CA (US); Raymond D. MENG, Mountain View, CA (US); Anh NGUYEN DUC, Basel (CH); Jilpa Bhupendra PATEL, Dublin, CA (US); Thinh Quang PHAM, Campbell, CA (US); Isabelle Anne ROONEY, Burlingame, CA (US); Heather Blythe STEVENS, San Francisco, CA (US); Sarah Marie TROUTMAN, San Francisco, CA (US); Lijia WANG, San Francisco, CA (US); Yulei WANG, Foster City, CA (US); Patrick Georges Robert WILLIAMS, San Francisco, CA (US); Benjamin WU, Millbrae, CA (US); Yibing YAN, San Francisco, CA (US); Aijing ZHANG, Hillsborough, NC (US); Xiaosong ZHANG, San Francisco, CA (US); Marcus Dale BALLINGER, Burlingame, CA (US); Hila BARAK, Belmont, CA (US); Elizabeth Alexandra BENNETT, South San Francisco, CA (US); Marcela Lucia CASTRO, San Francisco, CA (US); Edward Namserk CHA, San Carlos, CA (US); Hui Min Phyllis CHAN, San Francisco, CA (US); Stephen CHUI, South San Francisco, CA (US); Christopher Roland COTTER, Portland, OR (US); Viraj Vinay DEGAONKAR, South San Francisco, CA (US); Barbara Jennifer GITLITZ, Los Angeles, CA (US); Tien HOANG, Mill Valley, CA (US); Kimberly Mayumi KOMATSUBARA, Mountain View, CA (US)

(73) Assignees: Genentech, Inc., South San Francisco, CA (US); Hoffmann-La Roche Inc., Little Falls, NJ (US)

(21) Appl. No.: 17/159,128

(22) Filed: Jan. 26, 2021

Related U.S. Application Data
(60) Provisional application No. 63/127,109, filed on Dec. 17, 2020.
(60) Provisional application No. 63/124,693, filed on Dec. 11, 2020.
(60) Provisional application No. 63/114,517, filed on Nov. 16, 2020.
(60) Provisional application No. 63/105,198, filed on Oct. 23, 2020.
(60) Provisional application No. 63/085,890, filed on Sep. 30, 2020.
(60) Provisional application No. 63/074,827, filed on Sep. 4, 2020.
(60) Provisional application No. 63/074,807, filed on Sep. 4, 2020.
(60) Provisional application No. 63/059,960, filed on Jul. 31, 2020.
(60) Provisional application No. 63/059,054, filed on Jul. 30, 2020.
(60) Provisional application No. 62/994,272, filed on Mar. 24, 2020.
(60) Provisional application No. 62/985,822, filed on Mar. 5, 2020.
(60) Provisional application No. 62/966,448, filed on Jan. 27, 2020.

(51) Int. Cl. A61K 39/395; A61P 35/00; A61K 31/282; A61K 31/7048; A61K 31/519; A61K 31/7068; A61K 31/337; A61K 38/19; A61K 47/60; A61K 31/675; A61P 35/04; A61K 33/243; A61K 47/68; A61K 31/513; C07K 16/28

(52) U.S. Cl. CPC A61K 39/3955; A61K 2039/545; A61K 31/282; A61K 31/7048; A61K 31/519; A61K 31/7068; A61K 31/337; A61K 38/193; A61K 47/60; A61K 31/675; A61P 35/04; A61K 33/243; A61K 47/6801; A61K 47/6849; A61K 31/513; C07K 16/2809; C07K 16/2827; A61P 35/00

(57) ABSTRACT

The present invention relates to methods, uses, and compositions for the treatment of cancer (e.g., a lung cancer; a cervical cancer; a breast cancer; a head and neck cancer; a liver cancer; a bladder cancer; a gastric cancer; an esophageal cancer; a pancreatic cancer; a kidney or renal cancer; a melanoma; an ovarian cancer; or a colorectal cancer). More specifically, the invention concerns the treatment of patients having cancer with an anti-TIGIT antagonist antibody, including treatment with an anti-TIGIT antagonist antibody in a combination therapy.

### CROSS-REFERENCES TO RELATED APPLICATIONS

This application claims priority to U.S. Patent Application No. 62/966,448, filed on Jan. 27, 2020; U.S. Patent Application No. 62/985,822, filed on Mar. 5, 2020; U.S. Patent Application No. 62/994,272, filed on Mar. 24, 2020; U.S. Patent Application No. 63/059,054, filed on Jul. 30, 2020; U.S. Patent Application No. 63/059,960, filed on Jul. 31, 2020; U.S. Patent Application No. 63/074,807, filed on Sep. 4, 2020; U.S. Patent Application No. 63/074,827, filed on Sep. 4, 2020; U.S. Patent Application No. 63/085,890, filed on Sep. 30, 2020; U.S. Patent Application No. 63/105,198, filed on Oct. 23, 2020; U.S. Patent Application No. 63/114,517, filed on Nov. 16, 2020; U.S. Patent Application No. 63/124,693, filed on Dec. 11, 2020; and U.S. Patent Application No. 63/127,109, filed on Dec. 17, 2020, the entire contents of each of which are incorporated herein by reference in their entirety.

### SEQUENCE LISTING

The instant application contains a Sequence Listing which has been submitted electronically in ASCII format and is hereby incorporated by reference in its entirety. Said ASCII copy, created on Jan. 26, 2021, is named 50474-206002_Sequence_Listing_1_26_2021_ST25 and is 30,515 bytes in size.

### FIELD OF THE INVENTION

The present invention relates to methods, uses, and compositions for the treatment of cancer. More specifically, the invention concerns the treatment of patients having cancer with an anti-TIGIT antagonist antibody (e.g., treatment with an anti-TIGIT antagonist antibody as a monotherapy or a combination therapy).

### BACKGROUND OF THE INVENTION

Cancers are characterized by the uncontrolled growth of cell subpopulations. Cancers are the leading cause of death in the developed world and the second leading cause of death in developing countries, with over 14 million new cancer cases diagnosed and over eight million cancer deaths occurring each year. Cancer care thus represents a significant and ever-increasing societal burden.

Thus, there is an unmet need in the field for the development of efficacious immunotherapies and methods of dosing the same for the treatment of cancers.

### SUMMARY OF THE INVENTION

In one aspect, the invention provides a method of treating a subject having a cancer, the method comprising administering to the subject a dosing regimen comprising one or more dosing cycles of an anti-TIGIT antagonist antibody at a dose of about 500 mg to about 700 mg every three weeks, a PD-1 axis binding antagonist at a dose of about 900 mg to about 1500 mg every three weeks, a platinum-based chemotherapeutic agent every three weeks, and a non-platinum-based chemotherapeutic agent every three weeks.

In another aspect, the invention provides a method of treating a subject having a cancer, the method comprising administering to the subject a dosing regimen comprising one or more dosing cycles of an anti-TIGIT antagonist antibody at a dose of about 700 mg to about 1000 mg every four weeks and a PD-1 axis binding antagonist at a dose of about 1400 mg to 2000 mg every four weeks.

In another aspect, the invention provides a method of treating a subject having a cancer, the method comprising administering to the subject a dosing regimen comprising one or more dosing cycles of an anti-TIGIT antagonist antibody at a dose of about 300 mg to about 600 mg every two weeks and a PD-1 axis binding antagonist at a dose of about 600 mg to about 1200 mg every two weeks.

In another aspect, the invention provides a kit comprising an anti-TIGIT antagonist antibody for use in combination with a PD-1 axis binding antagonist for treating a subject having a cancer according to the methods provided herein.

In another aspect, the invention provides an anti-TIGIT antagonist antibody and a PD-1 axis binding antagonist for use in a method of treating a subject having a cancer, wherein the method is according to the methods provided herein.

In another aspect, the invention provides use of an anti-TIGIT antagonist antibody in the manufacture of a medicament for treating a subject having a cancer in combination with a PD-1 axis binding antagonist, wherein the treatment is according to the methods provided herein.

In another aspect, the invention provides a method of treating a subject having a cancer, the method comprising administering to the subject a dosing regimen comprising one or more dosing cycles of an anti-TIGIT antagonist antibody at a dose of about 700 mg to about 1000 mg every four weeks and a PD-1 axis binding antagonist at a dose of about 1400 mg to 2000 mg every four weeks.

In another aspect, the invention provides a method of treating a subject having a cancer, the method comprising administering to the subject a dosing regimen comprising one or more dosing cycles of an anti-TIGIT antagonist antibody at a dose of about 300 mg to about 600 mg every two weeks and a PD-1 axis binding antagonist at a dose of about 600 mg to about 1200 mg every two weeks.

In another aspect, the invention provides a method of treating a subject having a cancer, the method comprising administering to the subject a dosing regimen comprising one or more dosing cycles of an anti-TIGIT antagonist antibody at a dose of about 500 mg to about 700 mg every three weeks, a PD-1 axis binding antagonist at a dose of about 900 mg to about 1500 mg every three weeks, a platinum-based chemotherapeutic agent every three weeks, and a non-platinum-based chemotherapeutic agent every three weeks.

In another aspect, the invention provides a method of treating a subject having a cancer, the method comprising administering to the subject a dosing regimen comprising one or more dosing cycles of an anti-TIGIT antagonist antibody at a dose of about 500 mg to about 700 mg every three weeks and an anti-PD-1 antagonist antibody at a dose of about 100 mg to about 300 mg every three weeks, wherein the anti-PD-1 antagonist antibody is pembrolizumab.

In another aspect, the invention provides a method of treating a subject having a cancer, the method comprising administering to the subject a dosing regimen comprising one or more dosing cycles of tiragolumab and pembrolizumab, wherein the pembrolizumab is administered at a dose of between about 300 mg to about 500 mg every six weeks.

In another aspect, the invention provides a method for treating a subject having a cancer, the method comprising administering to the subject a dosing regimen comprising one or more dosing cycles of an anti-TIGIT antagonist antibody at a dose of between about 500 mg to about 700 mg every three weeks, a PD-1 axis binding antagonist at a dose of between about 900 mg to about 1500 mg every three weeks, and an antimetabolite at a dose of between about 10 mg/m² to about 10000 mg/m² twice a day orally every three weeks for 2-weeks on/1-week off.

In another aspect, the invention provides a method of treating a subject having a cancer, the method comprising administering to the subject a dosing regimen comprising one or more dosing cycles of an anti-TIGIT antagonist antibody at a dose of about 500 mg to about 700 mg every three weeks, a PD-1 axis binding antagonist at a dose of about 900 mg to about 1500 mg every three weeks, gemcitabine, and nab-paclitaxel.

In another aspect, the invention provides a method for treating a subject having a cancer, the method comprising administering to the subject a dosing regimen comprising one or more dosing cycles of an anti-TIGIT antagonist antibody at a dose of between about 500 mg to about 700 mg every three weeks, a PD-1 axis binding antagonist at a dose of between about 900 mg to about 1500 mg every three weeks, and a VEGF antagonist at a dose of between about 1 mg/kg to about 35 mg/kg every three weeks.

In another aspect, the invention provides a method of treating a subject having a cancer, the method comprising administering to the subject a dosing regimen comprising an induction phase and a maintenance phase, wherein (a) the induction phase comprises one or more dosing cycles of an anti-TIGIT antagonist antibody at a dose of about 500 mg to about 700 mg every three weeks, a PD-1 axis binding antagonist at a dose of about 900 mg to about 1500 mg every three weeks, a platinum-based chemotherapeutic agent every three weeks, and a non-platinum-based chemotherapeutic agent every three weeks; and (b) the maintenance phase comprises one or more additional dosing cycles of the anti-TIGIT antagonist antibody every three weeks, the PD-1 axis binding antagonist every three weeks, and the non-platinum-based chemotherapeutic agent every three weeks, and wherein the maintenance phase does not comprise administration of the platinum-based chemotherapeutic agent.

In another aspect, the invention provides a method of treating a subject having a cancer, the method comprising administering to the subject a dosing regimen comprising an induction phase and a maintenance phase, wherein (a) the induction phase comprises one or more dosing cycles of an anti-TIGIT antagonist antibody at a dose of about 500 mg to about 700 mg every three weeks, a PD-1 axis binding antagonist at a dose of about 900 mg to about 1500 mg every three weeks, a platinum-based chemotherapeutic agent every three weeks, and a non-platinum-based chemotherapeutic agent every three weeks; and (b) the maintenance phase comprises one or more additional dosing cycles of the anti-TIGIT antagonist antibody at a dose of about 700 mg to about 1000 mg every four weeks and the PD-1 axis binding antagonist at a dose of about 1400 mg to 2000 mg every four weeks, wherein the maintenance phase does not comprise administration of the platinum-based chemotherapeutic agent or non-platinum-based chemotherapeutic agent.

In another aspect, the invention provides a method of treating a subject or population of subjects having a lung cancer, the method comprising administering to the subject or population of subjects a dosing regimen comprising one or more dosing cycles of an effective amount of an anti-TIGIT antagonist antibody, a PD-1 axis binding antagonist, a platinum-based chemotherapeutic agent, and a topoisomerase II inhibitor, wherein the treatment extends progression-free survival (PFS) of the subject as compared to treatment with the PD-1 axis binding antagonist, the platinum-based chemotherapeutic agent, and the topoisomerase II inhibitor without the anti-TIGIT antagonist antibody.

In another aspect, the invention provides a method of treating a population of subjects having a lung cancer, the method comprising administering to the population of subjects a dosing regimen comprising one or more dosing cycles of an effective amount of an anti-TIGIT antagonist antibody, a PD-1 axis binding antagonist, a platinum-based chemotherapeutic agent, and a topoisomerase II inhibitor, wherein the treatment results in a median PFS of the population of subjects of about 8.2 months to about 9.2 months.

In another aspect, the invention provides a method of treating a subject or population of subjects having a lung cancer, the method comprising administering to the subject or population of subjects a dosing regimen comprising one or more dosing cycles of an effective amount of an anti-TIGIT antagonist antibody, a PD-1 axis binding antagonist, a platinum-based chemotherapeutic agent, and a topoisomerase II inhibitor, wherein the treatment extends OS of the subject as compared to treatment with the PD-1 axis binding antagonist, the platinum-based chemotherapeutic agent, and the topoisomerase II inhibitor without the anti-TIGIT antagonist antibody.

In another aspect, the invention provides a method of treating a population of subjects having a lung cancer, the method comprising administering to the population of subjects a dosing regimen comprising one or more dosing cycles of an effective amount of an anti-TIGIT antagonist antibody, a PD-1 axis binding antagonist, a platinum-based chemotherapeutic agent, and a topoisomerase II inhibitor, wherein the treatment results in a median OS of the population of subjects of about 15.3 months to about 17.6 months.

In another aspect, the invention provides a method for treating a subject or population of subjects having SCLC, the method comprising administering to the subject or population of subjects one or more 21-day dosing cycles of an anti-TIGIT antagonist antibody at a dose of about 500 mg to about 700 mg on Day 1 of each dosing cycle, atezolizumab at a dose of about 900 mg to about 1500 mg on Day 1 of each dosing cycle, carboplatin at a dose sufficient to achieve AUC=5 mg/ml/min on Day 1 of each dosing cycle, and etoposide at a dose of 100 mg/m² on each of Days 1, 2, and 3 of each dosing cycle, wherein the treatment extends PFS and/or OS of the subject or population of subjects as compared to treatment with atezolizumab, carboplatin, and etoposide without the anti-TIGIT antagonist antibody.

In another aspect, the invention provides a method for treating a subject or population of subjects having ES-SCLC, the method comprising administering to the subject or population of subjects four initial dosing cycles followed by one or more additional dosing cycles, wherein (a) the four initial dosing cycles comprise administering tiragolumab at a dose of about 600 mg on Day 1 of each initial dosing cycle, atezolizumab at a dose of about 1200 mg on Day 1 of each initial dosing cycle, carboplatin at a dose sufficient to achieve AUC=5 mg/ml/min on Day 1 of each initial dosing cycle, and etoposide at a dose of 100 mg/m² on each of Days 1, 2, and 3 of each initial dosing cycle; and (b) the one or more additional dosing cycles comprise administering tiragolumab at a dose of about 600 mg on Day 1 of each additional dosing cycle and atezolizumab at a dose of about 1200 mg on Day 1 of each additional dosing cycle, wherein the four initial dosing cycles and the one or more additional dosing cycles are each 21-day dosing cycles, and wherein the treatment extends PFS and/or OS of the subject or population of subjects as compared to treatment with atezolizumab, carboplatin, and etoposide without the tiragolumab.

In another aspect, the invention provides a method of treating a subject or population of subjects having a lung cancer, the method comprising administering to the subject or population of subjects a dosing regimen comprising one or more dosing cycles of an anti-TIGIT antagonist antibody, a PD-1 axis binding antagonist, a first chemotherapeutic agent which is a platinum-based chemotherapeutic agent, and a second chemotherapeutic agent which is a non-platinum-based chemotherapeutic agent.

In another aspect, the invention provides a method of treating a subject or population of subjects having an advanced non-squamous NSCLC, the method comprising administering to the subject or population of subjects a dosing regimen comprising four 21-day dosing cycles of tiragolumab, atezolizumab, carboplatin or cisplatin, and pemetrexed, wherein the tiragolumab is administered at a dose of about 600 mg every three weeks, the atezolizumab is administered at a dose of about 1200 mg every three weeks, the carboplatin is administered at a dose sufficient to achieve an AUC=5 mg/ml/min every three weeks or the cisplatin is administered at a dose of 75 mg/m² every three weeks, and the pemetrexed is administered at a dose of about 500 mg/m² every three weeks on Day 1 of each of the four 21-day dosing cycles.

In another aspect, the invention provides a method of treating a subject or population of subjects having an advanced non-squamous NSCLC, the method comprising administering to the subject or population of subjects (i) four induction phase dosing cycles of tiragolumab at a dose of about 600 mg every three weeks, atezolizumab at a dose of about 1200 mg every three weeks, carboplatin at a dose sufficient to achieve an AUC=5 mg/ml/min every three weeks, and pemetrexed at a dose of about 500 mg/m² every three weeks; and (ii) one or more maintenance phase dosing cycles of tiragolumab at a dose of about 600 mg every three weeks, atezolizumab at a dose of about 1200 mg every three weeks, and pemetrexed at a dose of about 500 mg/m² every three weeks, wherein the one or more 21-day dosing cycles of the maintenance phase do not comprise administration of the carboplatin, wherein the subject or population of subjects have received no prior systemic therapy for the advanced non-squamous NSCLC.

In another aspect, the invention provides a method for treating a subject having a resectable lung cancer, the method comprising administering to the subject one or more dosing cycles of an anti-TIGIT antagonist antibody at a dose of between about 500 mg to about 700 mg every three weeks and a PD-1 axis binding antagonist at a dose of between about 900 mg to about 1500 mg every three weeks.

In another aspect, the invention provides a method for treating a subject having a lung cancer, the method comprising administering to the subject one or more dosing cycles of an anti-TIGIT antagonist antibody and a PD-1 axis binding antagonist, wherein at least one of the dosing cycles comprises administering to the subject the anti-TIGIT antagonist antibody at a dose of between about 500 mg to about 700 mg every three weeks and the PD-1 axis binding antagonist at a dose of between about 900 mg to about 1500 mg every three weeks as a neoadjuvant treatment.

In another aspect, the invention provides a method for treating a subject having a resectable lung cancer, the method comprising administering to the subject one or more dosing cycles of tiragolumab at a dose of about 600 mg every three weeks, atezolizumab at a dose of about 1200 mg every three weeks, and (a) (i) carboplatin at a dose targeted to achieve an AUC of 5 mg/mL/min or an AUC of 6 mg/mL/min every three weeks; or (ii) cisplatin at a dose of about 75 mg/m² every three weeks; and (b) (i) pemetrexed at a dose of about 500 mg/m² every three weeks or gemcitabine at a dose of about 1000 mg/m² or about 1250 mg/m² on Days 1 and 8 of each dosing cycle; or (ii) paclitaxel at a dose of about 175 mg/m² or about 200 mg/m² every three weeks.

In another aspect, the invention provides a method for treating a subject having a lung cancer, the method comprising administering to the subject one or more dosing cycles of tiragolumab and atezolizumab, wherein (I) at least one of the dosing cycles is a neoadjuvant treatment and comprises administering to the subject (a) tiragolumab at a dose of about 1200 mg every three weeks; (b) atezolizumab at a dose of about 1200 mg every three weeks as a neoadjuvant treatment; and (c) (i) carboplatin at a dose targeted to achieve an AUC of 5 mg/mL/min every three weeks and gemcitabine at a dose of about 1000 mg/m² on Days 1 and 8 of each dosing cycle; (ii) carboplatin at a dose targeted to achieve an AUC of 6 mg/mL/min every three weeks and paclitaxel at a dose of about 175 mg/m² or about 200 mg/m² every three weeks; or (iii) cisplatin at a dose of about 75 mg/m² every three weeks and gemcitabine at a dose of about 1250 mg/m² on Days 1 and 8 of each dosing cycle; and (II) at least one of the dosing cycles comprises administering to the subject tiragolumab at a dose of between about 500 mg to about 700 mg every three weeks and atezolizumab at a dose of between about 900 mg to about 1500 mg every three weeks as an adjuvant treatment.

In another aspect, the invention provides a method for treating a subject or population of subjects having a cervical cancer with a detectable expression level of PD-L1, the method comprising administering to the subject or population of subjects one or more dosing cycles of an anti-TIGIT antagonist antibody at a dose of between about 500 mg to about 700 mg every three weeks and a PD-1 axis binding antagonist at a dose of between about 900 mg to about 1500 mg every three weeks.

In another aspect, the invention provides a method of selecting a therapy for a subject having a cervical cancer, the method comprising (a) detecting the protein expression level of PD-L1 on tumor cells from a tumor sample from the subject by an IHC assay using an anti-PD-L1 antibody suitable for staining; and (b) selecting for the subject having a detectable expression level of PD-L1 a therapy comprising one or more dosing cycles of an anti-TIGIT antagonist antibody administered at a dose of between about 500 mg to about 700 mg every three weeks and a PD-1 axis binding antagonist administered at a dose of between about 900 mg to about 1500 mg every three weeks based on PD-L1 expression on tumor cells having been detected.

In another aspect, the invention provides a method for treating a subject having a cervical cancer with a detectable expression level of PD-L1, the method comprising administering to the subject one or more dosing cycles of tiragolumab at a dose of about 600 mg every three weeks and atezolizumab at a dose of about 1200 mg every three weeks.

In another aspect, the invention provides a method of treating a subject or population of subjects having a breast cancer, the method comprising administering to the subject or population of subjects a dosing regimen comprising one or more dosing cycles of tiragolumab at a dose of about 840 mg every four weeks, atezolizumab at a dose of about 1680 mg every four weeks, and nab-paclitaxel at a dose of about 100 mg/m² for 3-weeks on/1-week off.

In another aspect, the invention provides a method of treating a subject having an early triple-negative breast cancer (eTNBC), the method comprising administering to the subject a dosing regimen comprising one or more dosing cycles of an anti-TIGIT antagonist antibody at a dose of about 300 mg to about 600 mg every two weeks and a PD-1 axis binding antagonist at a dose of about 600 mg to about 1200 mg every two weeks.

In another aspect, the invention provides a method of treating a subject having an eTNBC, the method comprising administering to the subject a dosing regimen comprising tiragolumab at a dose of about 420 mg every two weeks, atezolizumab at a dose of about 840 mg every two weeks, and (a) (i) nab-paclitaxel at a dose of about 125 mg/m² every week and carboplatin at a dose targeted to achieve an AUC of 5 mg/mL/min every three weeks for the first 12 weeks of the dosing regimen; and (ii) doxorubicin at a dose of about 60 mg/m² every two weeks, cyclophosphamide at a dose of about 600 mg/m² every two weeks, and G-CSF or GM-CSF every two weeks for weeks 13-19 of the dosing regimen; or (b) (i) nab-paclitaxel at a dose of about 125 mg/m² every week for the first 12 weeks of the dosing regimen; and (ii) doxorubicin at a dose of about 60 mg/m² every two weeks, cyclophosphamide at a dose of about 600 mg/m² every two weeks, and G-CSF or GM-CSF every two weeks for weeks 13-19 of the dosing regimen; wherein the method further comprises surgery between two and six weeks after the last dose of the dosing regimen.

In another aspect, the invention provides a method for treating a subject or population of subjects having an SCCHN with a detectable expression level of PD-L1, the method comprising administering to the subject or population of subjects one or more dosing cycles of an anti-TIGIT antagonist antibody at a dose of between about 500 mg to about 700 mg every three weeks and a PD-1 axis binding antagonist at a dose of between about 900 mg to about 1500 mg every three weeks.

In another aspect, the invention provides a method of selecting a therapy for a subject or population of subjects having an SCCHN, the method comprising: (a) detecting a protein expression level of PD-L1 in a tumor sample from the subject or population of subjects by an IHC assay using an anti-PD-L1 antibody suitable for staining; and (b) selecting for the subject or population of subjects having a detectable expression level of PD-L1 a therapy comprising one or more dosing cycles of a PD-1 axis binding antagonist at a dose of between about 900 mg to about 1500 mg every three weeks and an anti-TIGIT antagonist antibody at a dose of between about 500 mg to about 700 mg every three weeks based on PD-L1 expression having been detected.

In another aspect, the invention provides a method for treating a subject having an SCCHN with a detectable expression level of PD-L1, the method comprising administering to the subject one or more dosing cycles of tiragolumab at a dose of about 600 mg every three weeks and atezolizumab at a dose of about 1200 mg every three weeks.

In another aspect, the invention provides a method of treating a subject or population of subjects having a hepatocellular carcinoma (HCC), the method comprising administering to the subject or population of subjects one or more dosing cycles of an anti-TIGIT antagonist antibody and a PD-1 axis binding antagonist, wherein the subject or population of subjects have received no prior systemic treatment for HCC.

In another aspect, the invention provides a method of treating a subject or population of subjects having an HCC, the method comprising administering to the subject or population of subjects one or more dosing cycles of an anti-TIGIT antagonist antibody, a PD-1 axis binding antagonist, and a VEGF antagonist.

In another aspect, the invention provides a method of treating a subject or population of subjects having an HCC, the method comprising administering to the subject one or more dosing cycles of tiragolumab at a dose of about 600 mg every three weeks, atezolizumab at a dose of about 1200 mg every three weeks, and bevacizumab at a dose of about 15 mg/kg every three weeks.

In another aspect, the invention provides a method for treating a subject or population of subjects having an MIBC, the method comprising administering to the subject one or more dosing cycles of an anti-TIGIT antagonist antibody at a dose of between about 500 mg to about 700 mg every three weeks and a PD-1 axis binding antagonist at a dose of between about 900 mg to about 1500 mg every three weeks, wherein the subject is ineligible for treatment with a platinum-based chemotherapeutic agent.

In another aspect, the invention provides a method for treating a subject or population of subjects having an MIBC, the method comprising administering to the subject one or more dosing cycles of an anti-TIGIT antagonist antibody at a dose of between about 500 mg to about 700 mg every three weeks and a PD-1 axis binding antagonist at a dose of between about 900 mg to about 1500 mg every three weeks, wherein the treatment is a perioperative treatment.

In another aspect, the invention provides a method for treating a subject or population of subjects having an MIBC, the method comprising administering to the subject or population of subjects one or more dosing cycles of tiragolumab at a dose of about 600 mg every three weeks and atezolizumab at a dose of about 1200 mg every three weeks, wherein the subject or subjects are cisplatin ineligible.

In another aspect, the invention provides a method for treating a subject or population of subjects having an MIBC, the method comprising administering to the subject or population of subjects one or more dosing cycles of tiragolumab at a dose of about 600 mg every three weeks and atezolizumab at a dose of about 1200 mg every three weeks, wherein the treatment is a perioperative treatment.

In another aspect, the invention provides a method for treating a subject or population of subjects having an mUC, the method comprising administering to the subject or population of subjects a dosing regimen comprising one or more dosing cycles of an anti-TIGIT antagonist antibody at a dose of between about 500 mg to about 700 mg every three weeks and a PD-1 axis binding antagonist at a dose of between about 900 mg to about 1500 mg every three weeks.

In another aspect, the invention provides a method for treating a subject or population of subjects having an mUC, the method comprising administering to the subject or population of subjects one or more dosing cycles of tiragolumab at a dose of about 600 mg every three weeks and atezolizumab at a dose of about 1200 mg every three weeks.

In another aspect, the invention provides a method for treating a subject or population of subjects having an mUC, the method comprising administering to the subject or population of subjects a first dosing regimen followed by a second dosing regimen, wherein (a) the first dosing regimen comprises one or more dosing cycles of tiragolumab at a dose of about 600 mg every three weeks and atezolizumab at a dose of about 1200 mg every three weeks; and (b) the second dosing regimen comprises one or more dosing cycles of atezolizumab at a dose of about 1200 mg every three weeks and (i) enfortumab vedotin is administered at a dose of 1.25 mg/kg every week for 2-weeks on/1 week off or (ii) sacituzumab govitecan is administered at a dose of 10 mg/kg every week for 2-weeks on/1 week off, wherein the second dosing regimen is administered to the subject or population of subjects after the subject or population of subjects have experienced disease progression or unacceptable toxicity during the first dosing regimen.

In another aspect, the invention provides a method of treating a subject or population of subjects having a pancreatic cancer, the method comprising administering to the subject or population of subjects a dosing regimen comprising one or more 28-day dosing cycles of tiragolumab at a dose of about 420 mg on Days 1 and 15 of each 28-day dosing cycle, atezolizumab at a dose of about 840 mg on Days 1 and 15 of each 28-day dosing cycle, gemcitabine at a dose of about 1000 mg/m² on Days 1, 8, and 15 of each 28-day dosing cycle, and nab-paclitaxel at a dose of about 125 mg/m² on Days 1, 8, and 15 of each 28-day dosing cycle.

In another aspect, the invention provides a method for treating a subject or population of subjects having an advanced or metastatic esophageal cancer, the method comprising administering to the subject or population of subjects a dosing regimen comprising one or more 21-day dosing cycles of an anti-TIGIT antagonist antibody at a dose of between about 500 mg to about 700 mg on Day 1 of each dosing cycle and a PD-1 axis binding antagonist at a dose of between about 900 mg to about 1500 mg on Day 1 of each dosing cycle.

In another aspect, the invention provides a method for treating a subject or population of subjects having an esophageal cancer, the method comprising administering to the subject or population of subjects a dosing regimen comprising one or more 21-day dosing cycles of an anti-TIGIT antagonist antibody at a dose of between about 500 mg to about 700 mg on Day 1 of each dosing cycle and a PD-1 axis binding antagonist at a dose of between about 900 mg to about 1500 mg on Day 1 of each dosing cycle, wherein the subject or subjects have been previously treated with a platinum-based chemotherapeutic agent and a non-platinum-based chemotherapeutic agent.

In another aspect, the invention provides a method for treating a subject or population of subjects having an advanced or metastatic esophageal cancer, the method comprising administering to the subject or population of subjects a dosing regimen comprising one or more 21-day dosing cycles of tiragolumab at a dose of about 600 mg on Day 1 of each dosing cycle, atezolizumab at a dose of about 1200 mg on Day 1 of each dosing cycle, cisplatin at a dose of about 80 mg/m² on Day 1 of each dosing cycle, and 5-fluorouracil at a dose of 800 mg/m²/24 hours on Days 1-5 of each 21-day cycle, wherein cisplatin is omitted from the dosing regimen after six doses.

In another aspect, the invention provides a method for treating a subject or population of subjects having an advanced or metastatic esophageal cancer, the method comprising administering to the subject or population of subjects a first dosing regimen and a second dosing regimen, wherein (a) the first dosing regimen comprises one or more 21-day dosing cycles of cisplatin at a dose of about 80 mg/m² on Day 1 of each dosing cycle and 5-fluorouracil at a dose of 800 mg/m²/24 hours on Days 1-5 of each 21-day cycle, wherein cisplatin is omitted from the dosing regimen after six doses; and (b) the second dosing regimen comprises one or more 21-day dosing cycles of tiragolumab at a dose of about 600 mg on Day 1 of each dosing cycle and atezolizumab at a dose of about 1200 mg on Day 1 of each dosing cycle.

In another aspect, the invention provides a kit comprising a PD-1 axis binding antagonist and/or an anti-TIGIT antagonist antibody for treating a subject having a cancer according to the methods provided herein.

In another aspect, the invention provides a kit comprising a PD-1 axis binding antagonist for use in combination with an anti-TIGIT antagonist antibody for treating a subject having a cancer according to the methods provided herein.

In another aspect, the invention provides a kit comprising an anti-TIGIT antagonist antibody for use in combination with a PD-1 axis binding antagonist for treating a subject having a cancer according to the methods provided herein.

In another aspect, the invention provides an anti-TIGIT antagonist antibody and a PD-1 axis binding antagonist for use in a method of treating a subject or population of subjects having a cancer, wherein the method is according to the methods provided herein.

In another aspect, the invention provides use of an anti-TIGIT antagonist antibody in the manufacture of a medicament for treating a subject or population of subjects having a cancer in combination with a PD-1 axis binding antagonist, wherein the treatment is according to the methods provided herein.

### BRIEF DESCRIPTION OF THE DRAWINGS

FIG. 1 is a flow chart showing the Phase Ib chemotherapy expansion and Phase Ib Q4W dosing expansion).

FIG. 2 is a flow chart of a phase Ib trial schema. EOCG=Eastern Cooperative Oncology Group; IHC=immunohistochemistry; PD-L1+=programmed death-ligand 1 positive; Q4W=every 4 weeks; RECIST v1.1=Response Criteria in Solid Tumors, Version 1.1; TFI=treatment-free interval; TNBC=triple-negative breast cancer.

FIG. 3 is a flow chart of a phase Ia study design. CRC=colorectal cancer, DLT=dose-limiting toxicity, GC=gastric cancer, HNSCC=head and neck squamous cell carcinoma, IV=intravenous, MAD=maximum administered dose, MSI=microsatellite instability, MSS=microsatellite-stable, MTD=maximum tolerated dose, NSCLC=non-small cell lung cancer, OC=ovarian cancer, PD=progression of disease, PK=pharmacokinetic, RCC=renal cell carcinoma, TNBC=triple-negative breast cancer, UBC=urothelial bladder cancer.

FIG. 4 is a flow chart of the phase Ib tiragolumab and atezolizumab expansion cohorts, serial biopsy cohort, and Q4W dosing expansion cohort study design. CIT=cancer immunotherapy; DLT=dose-limiting toxicity; HNSCC=head and neck squamous cell carcinoma, IV=intravenous, MAD=maximum administered dose, MSI=microsatellite instability, MSS=microsatellite-stable, MTD=maximum tolerated dose, NSCLC=non-small cell lung cancer, PD=progression of disease; PD 1=programmed death-1; PD-L1=programmed death ligand 1; PK=pharmacokinetic, TIGIT=T-cell Immunoreceptor with Ig and ITIM domains. Note: Limited to patients with PD-L1 selected and/or TIGIT-selected tumors. Advanced, incurable, refractory tumors. Tiragolumab IV and atezolizumab 1200 mg IV every 3 weeks. Will only enroll following IMC review of safety data from Phase Ia. 3+3 dose escalation with DLT window of 21 days. Other intermediate doses of tiragolumab may be studied if they do not exceed the MTD. To gain further safety, PK, and PD data, patients may be backfill-enrolled if they consent to optional serial biopsies. Evaluation of dose levels exceeding 1200 mg tiragolumab will require a protocol amendment with a supporting rationale. ^{b}Expansion may begin at doses ≤MTD or MAD. ^{c}Up to approximately half of patients in each cohort will be those who consent to optional serial biopsies. Patients with tumors for which anti-PD-L1/PD-1 agents are approved by local regulatory authorities (e.g., NSCLC melanoma, renal cell carcinoma) may only be enrolled in an expansion cohort (indication-specific or serial biopsy) if a clinical trial of an investigational agent in combination with an anti-PD-L1 is considered an acceptable treatment option. ^{d}Limited to patients who consent to optional serial biopsies (acceptable samples include core needle, excisional, incisional, punch, and/or foreceps biopsies). ^{e}In the Q4W dosing expansion cohort, a safety run-in of approximately 3 patients will be completed. All relevant safety data from the safety run-in will be thoroughly reviewed by an IMC and by the investigators before enrollment is continued.

FIG. 5 is a flow chart of the phase Ib chemotherapy expansion cohorts study design.

FIG. 6 is a flow chart of the phase Ib non-chemotherapy expansion cohorts study design.

FIG. 7 is a flow chart showing the conditions for continuing study treatment beyond progression. ECOG=Eastern Cooperative Oncology Group; RECIST=Response Evaluation Criteria in Solid Tumors.

FIG. 8 is a flow chart showing crossover from phase Ia to phase Ib. AE=adverse event, DLT=dose-limiting toxicity, PD=progression of disease.

FIG. 9 is a graph showing the pharmacokinetics of tiragolumab.

FIG. 10 is a series of graphs showing the pharmacodynamics of tiragolumab.

FIG. 11 is a graph showing all adverse events 10% in a phase Ia tiragolumab dose-escalation study. *Grade 5 AEs were malignant neoplasm progression (n=3), not related to tiragolumab.

FIG. 12 is a graph showing all adverse events 10% in phase Ib tiragolumab and atezolizumab dose-escalation study. *Grade 5 AEs were malignant neoplasm progression (n=12) and pulmonary embolism (n=2), not related to study drug(s).

FIG. 13 is a graph showing tumor size reduction in phase Ia tiragolumab dose-escalation study.

FIG. 14 is a graph showing tumor size reduction in phase Ib tiragolumab and atezolizumab dose-escalation study.

FIG. 15 is a graph showing CIT-naive PD-L1-positive NSCLC tumor size reduction in phase Ib tiragolumab and atezolizumab dose-escalation study.

FIG. 16 is a graph showing CIT-naive PD-L1-positive NSCLC tumor size reduction over time in phase Ib tiragolumab and atezolizumab dose-escalation study.

FIG. 17 is a flow chart of a phase III trial schema. 1L=first-line; CE=carboplatin and etoposide; ECOG PS=Eastern Cooperative Oncology Group performance status; ES-SCLC=extensive-stage small cell lung cancer; LDH=lactase dehydrogenase; RECIST=Response Evaluation Criteria in Solid Tumors; ITT=intent-to-treat; PP=primary population.

FIG. 18 is a schematic diagram of the study design showing the parameters for the selection of subjects, stratification criteria, randomization into treatment arms, and treatment endpoints.

FIG. 19 is a schematic diagram showing the design of the GO42501 Phase II clinical trial. Patients with resectable Stage II, IIIA, or select IIIB (T3N2) non-small cell lung cancer (NSCLC) who do not have an activating EGFR mutation (EGFR−), do not have an ALK fusion oncogene (ALK−), and have an Eastern Cooperative Oncology Group (ECOG) Performance Status of 0 or 1, are selected. Cohort A consists of PD-L1 high patients. Patients in Cohort A are treated with atezolizumab (Atezo) and tiragolumab (Tira) every three weeks (Q3W) for 4 cycles. Cohort B consists of patients having any PD-L1 status. Patients in Cohort B are treated with Atezo, Tira, and platinum-based doublet chemotherapy (Chemo) Q3W for 4 cycles. Chest computed tomography (CT) is performed after Cycle 2 and Cycle 4. Surgery is performed for patients in both cohorts, and major pathological response (MPR) and pathological complete response (pCR) are assessed. Following surgery, patients in Cohort A are treated with Atezo and Tira Q3W for 16 cycles or Chemo Q3W for 4 cycles. Patients in Cohort B are treated with Atezo and Tira Q3W for 16 cycles. Postoperative radiotherapy (PORT) is optional for R1/R2 resections and/or ypN2 prior to adjuvant administration. Survival follow-up is performed.

FIG. 20 is a flow chart showing patient enrollment in the GO42501 Phase II clinical trial. For Cohort A (PD-L1 high patients; PD-L1 tumor proportion score (TPS)≥50%), a safety lead-in is performed using 6 patients. If surgical safety criteria are not met, enrollment of Cohort A is halted. If surgical safety criteria are met, enrollment of Cohort A is continued. For Cohort B (patients with any PD-L1 status), a safety lead-in is performed using 6 patients with PD-L1 TPS <50%. If surgical safety criteria are not met, enrollment of Cohort B is halted. If surgical safety criteria are met, enrollment of Cohort B is continued, and patients having PD-L1 TPS <50% and PD-L1 TPS 50% are enrolled. After 8 patients with tumors expressing PD-L1 ≥50% have been enrolled into Cohort B, enrollment of patients having PD-L1 TPS ≥50% into Cohort A is continued.

FIG. 21 is a flow chart of a phase II trial schema. 1L=first line; CDx=companion diagnostic; ECOG PS=Eastern Cooperative Oncology Group performance status; IHC=immunohistochemistry; IRC=independent review committee; PD-L1=programmed death-ligand 1; Q3W=every 3 weeks; R=randomization; s/p=status-post; IV=intravenous.

FIG. 22 is a flow chart of a Phase Ib trial schema. TNBC=triple-negative breast cancer; G-CSF=granulocyte colony-stimulating factor; GM-CSF=granulocyte-macrophage colony-stimulating factor; Rand.=randomization, pCR=pathologic complete response, QW=once a week, Q2W=every two weeks, Q3W=every three weeks.

FIG. 23 is a flow chart of a Phase II trial schema. HPV=Human papillomavirus; IHC=immunohistochemistry; IV=intravenous; Q3W=every 3 weeks; PD-L1 low=TIC 10%-49%; PD-L1 high=TIC ≥50%; SCCHN=squamous cell carcinoma of head and neck; RECIST=Response Evaluation Criteria in Solid Tumors.

FIG. 24 is a schematic diagram showing the design of the muscle-invasive bladder cancer (MIBC) cohorts of the WO039613 Phase Ib/II clinical trial. At the screening stage, patients having MIBC who are cisplatin ineligible and either PD-L1 positive (+) (top) or PD-L1 negative (−) (bottom) are identified. Following randomization (R), patients are treated with atezolizumab (Atezo; control) or atezolizumab and tiragolumab (Tira).

FIG. 25 is a schematic diagram showing the design of the metastatic urothelial carcinoma (mUC) cohorts of the WO039613 Phase Ib/II clinical trial. At the screening stage, patients having second-line locally advanced or metastatic UC who have progressed during or following platinum-containing therapy and who are cancer immunotherapy (CIT) naïve are identified. Following randomization (R), patients are treated with atezolizumab (Atezo; control); atezolizumab and enfortumab vedotin (EV); atezolizumab and niraparib (Nira); atezolizumab and Hu5F9-G4; atezolizumab and tiragolumab (Tira); atezolizumab and sacituzumab govitecan (SG); atezolizumab and tocilizumab (TCZ); atezolizumab and RO7122290 (FAP-4-1 BBL); or RO7121661 (PD1/TIM-3) during Stage 1. ^{a}During Stage 1, patients who experience loss of clinical benefit, as determined by the investigator, or unacceptable toxicity may be eligible to receive a different treatment combination during Stage 2, provided they meet the eligibility criteria. ^{b}The Atezo+Nira arm has 40 patients. Patients who received enfortumab vedotin in Stage 1 do not receive enfortumab vedotin in Stage 2, and patients who received sacituzumab govitecan in Stage 1 do not receive sacituzumab govitecan in Stage 2; other patients who are eligible for more than one treatment arm are assigned a treatment arm by the investigator. Once the Atezo+SG arm opens, enrollment in the Atezo+EV arm is closed. ^{d}The Atezo+RO7122290 (FAP-4-1 BBL) arm is only opened in ex-U.S. countries.

FIG. 26 is a diagram showing the objective response rate (ORR) (complete response/partial response (CR/PR); stable disease/progressive disease (SD/PD); or not evaluable (NE)) in patients from the CITYSCAPE trial having low or high PD-L1 TPS as assessed by the pharmDx 22C3 IHC assay (high TPS ≥50%; low TPS 1-49%) or low or high PD-L1 tumor content (TC) as assessed by the CE-IVD VENTANA SP263 IHC assay (high TC ≥50%; low TC 1-49%).

FIG. 27A is a bar graph showing the response rate (95% confidence interval (CI)) for patients from the CITYSCAPE trial having a TPS ≥1% as measured using the 22C3 IHC assay.

FIG. 27B is a bar graph showing the response rate (95% CI) for patients from the CITYSCAPE trial having a TC ≥1% as measured using the SP263 IHC assay (and TPS ≥1% as measured using the 22C3 IHC assay).

FIG. 28A is a graph showing progression-free survival (percent) for patients from the CITYSCAPE trial who were treated with tiragolumab and atezolizumab (tira+atezo) or placebo+atezo and had a TPS ≥1% as measured using the 22C3 IHC assay. The inset table shows median PFS in months (mo) and hazard ratio (HR).

FIG. 28B is a graph showing progression-free survival (percent) for patients from the CITYSCAPE trial who were treated with tiragolumab and atezolizumab (tira+atezo) or placebo+atezo and had a TC ≥1% as measured using the SP263 IHC assay (and TPS ≥1% as measured using the 22C3 IHC assay). The inset table shows median PFS in months and HR.

FIG. 29A is a bar graph showing the response rate (95% confidence interval (CI)) for patients from the CITYSCAPE trial having a TPS ≥50% as measured using the 22C3 IHC assay.

FIG. 29B is a bar graph showing the response rate (95% CI) for patients from the CITYSCAPE trial having a TC ≥50% as measured using the SP263 IHC assay.

FIG. 30A is a graph showing progression-free survival (percent) for patients from the CITYSCAPE trial who were treated with tiragolumab and atezolizumab (tira+atezo) or placebo+atezo and had a TPS ≥50% as measured using the 22C3 IHC assay. The inset table shows median PFS in months and HR.

FIG. 30B is a graph showing progression-free survival (percent) for patients from the CITYSCAPE trial who were treated with tiragolumab and atezolizumab (tira+atezo) or placebo+atezo and had a TC ≥50% as measured using the SP263 IHC assay. The inset table shows median PFS in months and HR.

### DETAILED DESCRIPTION OF THE INVENTION

The present invention provides therapeutic methods and compositions for treatment of cancer (e.g., a lung cancer (e.g., an early stage lung cancer (e.g., a resectable lung cancer), a SCLC (e.g., an ES-SCLC), a NSCLC (e.g., a squamous NSCLC or a non-squamous NSCLC, a locally advanced unresectable NSCLC, a Stage IIIB NSCLC, a recurrent or metastatic NSCLC (e.g., a locally advanced unresectable or metastatic non-squamous NSCLC (e.g., Stage IV non-squamous NSCLC)), or a Stage IV NSCLC (e.g., wherein the subject has not been previously treated for Stage IV NSCLC))); a cervical cancer (e.g., a Stage IVB, metastatic, recurrent, or persistent cervical cancer, e.g., a metastatic and/or recurrent PD-L1-positive cervical carcinoma); a breast cancer (e.g., a TNBC (e.g., an early TNBC (eTNBC))) or a HER2-positive breast cancer); a head and neck cancer (e.g., SCCHN, e.g., recurrent/metastatic PD-L1-positive SCCHN); a liver cancer (e.g., HCC, e.g., locally advanced or metastatic HCC and/or unresectable HCC); a bladder cancer (e.g., MIBC, locally advanced UC, or mUC); an esophageal cancer; a pancreatic cancer (e.g., PDAC, e.g., metastatic PDAC); a kidney or renal cancer (e.g., a RCC); a melanoma; an ovarian cancer; a gastric cancer (e.g., a gastroesophageal junction cancer); or a CRC (e.g., MSS or MSI-Low CRC)). The invention is based, at least in part, on the discovery that immunotherapies including an anti-TIGIT antibody (e.g., an anti-TIGIT antagonist antibody, such as tiragolumab) in combination with a PD-1 axis binding antagonist, a VEGF antagonist, and/or a chemotherapeutic agent can be useful in the treatment of cancer. Compositions, uses, and kits involving such combinations and/or dosing regimens are also provided herein.

### I. General Techniques

The techniques and procedures described or referenced herein are generally well understood and commonly employed using conventional methodology by those skilled in the art, such as, for example, the widely utilized methodologies described in Sambrook et al., Molecular Cloning: A Laboratory Manual 3d edition (2001) Cold Spring Harbor Laboratory Press, Cold Spring Harbor, N.Y.; Current Protocols in Molecular Biology (F. M. Ausubel, et al. eds., (2003)); the series Methods in Enzymology (Academic Press, Inc.): PCR 2: A Practical Approach (M. J. MacPherson, B. D. Hames and G. R. Taylor eds. (1995)), Harlow and Lane, eds. (1988) Antibodies, A Laboratory Manual, and Animal Cell Culture (R. I. Freshney, ed. (1987)); Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney), ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-8) J. Wiley and Sons; Handbook of Experimental Immunology (D. M. Weir and C. C. Blackwell, eds.); Gene Transfer Vectors for Mammalian Cells (J. M. Miller and M. P. Calos, eds., 1987); PCR: The Polymerase Chain Reaction, (Mullis et al., eds., 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: A Practical Approach (D. Catty., ed., IRL Press, 1988-1989); Monoclonal Antibodies: A Practical Approach (P. Shepherd and C. Dean, eds., Oxford University Press, 2000); Using Antibodies: A Laboratory Manual (E. Harlow and D. Lane (Cold Spring Harbor Laboratory Press, 1999); The Antibodies (M. Zanetti and J. D. Capra, eds., Harwood Academic Publishers, 1995); and Cancer: Principles and Practice of Oncology (V. T. DeVita et al., eds., J.B. Lippincott Company, 1993).

### II. Definitions

It is to be understood that aspects and embodiments of the invention described herein include “comprising,” “consisting,” and “consisting essentially of” aspects and embodiments. As used herein, the singular form “a,” “an,” and “the” includes plural references unless indicated otherwise.

The term “about” as used herein refers to the usual error range for the respective value readily known to the skilled person in this technical field. Reference to “about” a value or parameter herein includes (and describes) aspects that are directed to that value or parameter per se. For example, description referring to “about X” includes description of “X.”

The terms “level of expression” or “expression level” in general are used interchangeably and generally refer to the amount of a biomarker in a biological sample. “Expression” generally refers to the process by which information (e.g., gene-encoded and/or epigenetic information) is converted into the structures present and operating in the cell. Therefore, as used herein, “expression” may refer to transcription into a polynucleotide, translation into a polypeptide, or even polynucleotide and/or polypeptide modifications (e.g., posttranslational modification of a polypeptide). Fragments of the transcribed polynucleotide, the translated polypeptide, or polynucleotide and/or polypeptide modifications (e.g., posttranslational modification of a polypeptide) shall also be regarded as expressed whether they originate from a transcript generated by alternative splicing or a degraded transcript, or from a post-translational processing of the polypeptide, e.g., by proteolysis. “Expressed genes” include those that are transcribed into a polynucleotide as mRNA and then translated into a polypeptide, and also those that are transcribed into RNA but not translated into a polypeptide (for example, transfer and ribosomal RNAs). An “amount” or “level” (e.g., expression level) of a biomarker can be measured by methods known to one skilled in the art and also disclosed herein. The amount or level of a biomarker associated with an increased clinical benefit to an individual can, for example, be a detectable level in a biological sample. In some aspects, the expression level or amount of a biomarker can be used to identify/characterize a subject having a cancer who may be likely to respond to, or benefit from, a particular therapy (e.g., a therapy comprising one or more dosing cycles of a PD-1 axis binding antagonist and an anti-TIGIT antagonist antibody or a therapy comprising one or more dosing cycles of an anti-TIGIT antagonist antibody).

“Increased expression,” “increased expression level,” “increased levels,” “elevated expression,” “elevated expression levels,” or “elevated levels” refers to an increased expression or increased levels of a biomarker in an individual relative to a control, such as an individual or individuals who are not suffering from the disease or disorder (e.g., cancer) or an internal control (e.g., a housekeeping biomarker).

“Decreased expression,” “decreased expression level,” “decreased levels,” “reduced expression,” “reduced expression levels,” or “reduced levels” refers to a decrease expression or decreased levels of a biomarker in an individual relative to a control, such as an individual or individuals who are not suffering from the disease or disorder (e.g., cancer) or an internal control (e.g., a housekeeping biomarker). In some aspects, reduced expression is little or no expression.

The presence and/or expression level/amount of various biomarkers described herein in a sample can be analyzed by a number of methodologies, many of which are known in the art and understood by the skilled artisan, including, but not limited to, immunohistochemistry (“IHC”), Western blot analysis, immunoprecipitation, molecular binding assays, ELISA, ELIFA, flow cytometry, fluorescence activated cell sorting (“FACS”), MassARRAY, proteomics, quantitative blood based assays (e.g., Serum ELISA), biochemical enzymatic activity assays, in situ hybridization (ISH), fluorescence in situ hybridization (FISH), Southern analysis, Northern analysis, whole genome sequencing, massively parallel DNA sequencing (e.g., next-generation sequencing), NANOSTRING®, polymerase chain reaction (PCR) including quantitative real time PCR (qRT-PCR) and other amplification type detection methods, such as, for example, branched DNA, SISBA, TMA and the like, RNA-seq, microarray analysis, gene expression profiling, and/or serial analysis of gene expression (“SAGE”), as well as any one of the wide variety of assays that can be performed by protein, gene, and/or tissue array analysis. Typical protocols for evaluating the status of genes and gene products are found, for example in Ausubel et al., eds., 1995, Current Protocols In Molecular Biology, Units 2 (Northern Blotting), 4 (Southern Blotting), 15 (Immunoblotting) and 18 (PCR Analysis). Multiplexed immunoassays such as those available from Rules Based Medicine or Meso Scale Discovery (“MSD”) may also be used.

By “correlate” or “correlating” is meant comparing, in any way, the performance and/or results of a first analysis or protocol with the performance and/or results of a second analysis or protocol. For example, one may use the results of a first analysis or protocol in carrying out a second protocol and/or one may use the results of a first analysis or protocol to determine whether a second analysis or protocol should be performed. With respect to the aspect of polypeptide analysis or protocol, one may use the results of the polypeptide expression analysis or protocol to determine whether a specific therapeutic regimen should be performed. With respect to the aspect of polynucleotide analysis or protocol, one may use the results of the polynucleotide expression analysis or protocol to determine whether a specific therapeutic regimen should be performed.

The phrase “substantially reduced” or “substantially different,” as used herein, denotes a sufficiently high degree of difference between two numeric values (generally one associated with a molecule and the other associated with a reference/comparator molecule) such that one of skill in the art would consider the difference between the two values to be of statistical significance within the context of the biological characteristic measured by said values (e.g., K_{D} values). The difference between said two values is, for example, greater than about 10%, greater than about 20%, greater than about 30%, greater than about 40%, and/or greater than about 50% as a function of the value for the reference/comparator molecule.

The term “substantially similar” or “substantially the same,” as used herein, denotes a sufficiently high degree of similarity between two numeric values (for example, one associated with an antibody of the invention and the other associated with a reference/comparator antibody), such that one of skill in the art would consider the difference between the two values to be of little or no biological and/or statistical significance within the context of the biological characteristic measured by said values (e.g., K_{D} values). The difference between said two values is, for example, less than about 50%, less than about 40%, less than about 30%, less than about 20%, and/or less than about 10% as a function of the reference/comparator value.

The phrase “based on” when used herein means that the information about one or more biomarkers is used to inform a treatment decision, information provided on a package insert, or marketing/promotional guidance, and the like.

The term “TIGIT” or “T-cell immunoreceptor with Ig and ITIM domains” as used herein refers to any native TIGIT from any vertebrate source, including mammals such as primates (e.g., humans) and rodents (e.g., mice and rats), unless otherwise indicated. TIGIT is also known in the art as DKFZp667A205, FLJ39873, V-set and immunoglobulin domain-containing protein 9, V-set and transmembrane domain-containing protein 3, VSIG9, VSTM3, and WUCAM. The term encompasses “full-length,” unprocessed TIGIT (e.g., full-length human TIGIT having the amino acid sequence of SEQ ID NO: 30), as well as any form of TIGIT that results from processing in the cell (e.g., processed human TIGIT without a signal sequence, having the amino acid sequence of SEQ ID NO: 31). The term also encompasses naturally occurring variants of TIGIT, e.g., splice variants or allelic variants. The amino acid sequence of an exemplary human TIGIT may be found under UniProt Accession Number Q495A1.

The terms “programmed death ligand 1” and “PD-L1” refer herein to native sequence human PD-L1 polypeptide. Native sequence PD-L1 polypeptides are provided under Uniprot Accession No. Q9NZQ7 (SEQ ID NO: 32). For example, the native sequence PD-L1 may have the amino acid sequence as set forth in Uniprot Accession No. Q9NZQ7-1 (isoform 1). In another example, the native sequence PD-L1 may have the amino acid sequence as set forth in Uniprot Accession No. Q9NZQ7-2 (isoform 2). In yet another example, the native sequence PD-L1 may have the amino acid sequence as set forth in Uniprot Accession No. Q9NZQ7-3 (isoform 3). The term also encompasses naturally occurring variants of PD-L1, e.g., splice variants, or allelic variants. PD-L1 is also referred to in the art as “programmed cell death 1 ligand 1,” “PDCD1LG1,” “CD274,” “B7-H,” and “PDL1.”

The term “PD-1” or “Programmed Cell Death protein 1” refers herein to any native PD-1 from any vertebrate source, including mammals such as primates (e.g., humans) and rodents (e.g., mice and rats), unless otherwise indicated. PD-1 is also known in the art as CD279, PDCD1, and programmed cell death 1. The term also encompasses naturally occurring variants of PD-1, e.g., splice variants, or allelic variants. The amino acid sequence of an exemplary human PD-1 may be found under UniProt Accession Number Q15116.

The term “PD-L2” or “Programmed Cell Death 1 Ligand 2” refers herein to any native PD-L2 from any vertebrate source, including mammals such as primates (e.g., humans) and rodents (e.g., mice and rats), unless otherwise indicated. PD-L2 is also known in the art as CD273 molecule, B7DC, and PDCD1 L2. The term also encompasses naturally occurring variants of PD-L2, e.g., splice variants, or allelic variants. The amino acid sequence of an exemplary human PD-L2 may be found under UniProt Accession Number Q9BQ51.

The term “antagonist” is used in the broadest sense, and includes any molecule that partially or fully blocks, inhibits, or neutralizes a biological activity of a native polypeptide disclosed herein. Suitable antagonist molecules specifically include antagonist antibodies or antibody fragments (e.g., antigen-binding fragments), fragments or amino acid sequence variants of native polypeptides, peptides, antisense oligonucleotides, small organic molecules, etc. Methods for identifying antagonists of a polypeptide may comprise contacting a polypeptide with a candidate antagonist molecule and measuring a detectable change in one or more biological activities normally associated with the polypeptide.

The term “PD-1 axis binding antagonist” refers to a molecule that inhibits the interaction of a PD-1 axis binding partner with either one or more of its binding partners, so as to remove T-cell dysfunction resulting from signaling on the PD-1 signaling axis, with a result being to restore or enhance T-cell function (e.g., proliferation, cytokine production, and/or target cell killing). As used herein, a PD-1 axis binding antagonist includes a PD-L1 binding antagonist, a PD-1 binding antagonist, and a PD-L2 binding antagonist. In some instances, the PD-1 axis binding antagonist includes a PD-L1 binding antagonist or a PD-1 binding antagonist. In one aspect, the PD-1 axis binding antagonist is a PD-L1 binding antagonist. In another aspect, the PD-1 axis binding antagonist is a PD-1 binding antagonist. In another aspect, the PD-1 axis binding antagonist is a PD-L2 binding antagonist.

The term “PD-1 binding antagonist” refers to a molecule that decreases, blocks, inhibits, abrogates or interferes with signal transduction resulting from the interaction of PD-1 with one or more of its binding partners, such as PD-L1 and/or PD-L2. PD-1 (programmed death 1) is also referred to in the art as “programmed cell death 1,” “PDCD1,” “CD279,” and “SLEB2.” An exemplary human PD-1 is shown in UniProtKB/Swiss-Prot Accession No. Q15116. In some instances, the PD-1 binding antagonist is a molecule that inhibits the binding of PD-1 to one or more of its binding partners. In a specific aspect, the PD-1 binding antagonist inhibits the binding of PD-1 to PD-L1 and/or PD-L2. For example, PD-1 binding antagonists include anti-PD-1 antibodies, antigen-binding fragments thereof, immunoadhesins, fusion proteins, oligopeptides, and other molecules that decrease, block, inhibit, abrogate or interfere with signal transduction resulting from the interaction of PD-1 with PD-L1 and/or PD-L2. In one instance, a PD-1 binding antagonist reduces the negative co-stimulatory signal mediated by or through cell surface proteins expressed on T lymphocytes mediated signaling through PD-1 so as render a dysfunctional T-cell less dysfunctional (e.g., enhancing effector responses to antigen recognition). In some instances, the PD-1 binding antagonist binds to PD-1. In some instances, the PD-1 binding antagonist is an anti-PD-1 antibody (e.g., an anti-PD-1 antagonist antibody). Exemplary anti-PD-1 antagonist antibodies include nivolumab, pembrolizumab, MEDI-0680 (AMP 514), PDR001 (spartalizumab), REGN2810 (cemiplimab), BGB-108, prolgolimab, camrelizumab, sintilimab, tislelizumab, toripalimab, dostarlimab, retifanlimab, sasanlimab, penpulimab, CS1003, HLX10, SCT-110A, zimberelimab, balstilimab, genolimzumab, BI 754091, cetrelimab, YBL-006, BAT1306, HX008, budigalimab, AMG 404, CX-188, JTX-4014, 609A, Sym021, LZM009, F520, SG001, AM0001, ENUM 244C8, ENUM 388D4, STI-1110, AK-103, and hAb21. In a specific aspect, a PD-1 binding antagonist is MDX-1106 (nivolumab). In another specific aspect, a PD-1 binding antagonist is MK-3475 (pembrolizumab, previously known as lambrolizumab). In another specific aspect, a PD-1 binding antagonist is a PD-L2 Fc fusion protein, e.g., AMP-224. In another specific aspect, a PD-1 binding antagonist is MEDI-0680. In another specific aspect, a PD-1 binding antagonist is PDR001 (spartalizumab). In another specific aspect, a PD-1 binding antagonist is REGN2810 (cemiplimab). In another specific aspect, a PD-1 binding antagonist is BGB-108. In another specific aspect, a PD-1 binding antagonist is prolgolimab. In another specific aspect, a PD-1 binding antagonist is camrelizumab. In another specific aspect, a PD-1 binding antagonist is sintilimab. In another specific aspect, a PD-1 binding antagonist is tislelizumab. In another specific aspect, a PD-1 binding antagonist is toripalimab. Other additional exemplary PD-1 binding antagonists include BION-004, CB201, AUNP-012, ADG104, and LBL-006.

The term “anti-PD-1 antagonist antibody” refers to an antibody or an antigen-binding fragment or variant thereof that is capable of binding PD-1 with sufficient affinity such that it substantially or completely inhibits the biological activity of PD-1. For example, an anti-PD-1 antagonist antibody may decrease, block, inhibit, abrogate or interfere with signal transduction resulting from the interaction of PD-1 with either one or more of its binding partners, such as PD-L1 and/or PD-L2. It will be understood by one of ordinary skill in the art that in some instances, an anti-PD-1 antagonist antibody may antagonize one PD-1 activity without affecting another PD-1 activity. For example, an anti-PD-1 antagonist antibody for use in certain of the methods or uses described herein is an anti-PD-1 antagonist antibody that antagonizes PD-1 activity in response to one of its binding partners (e.g., PD-L1 or PD-L2) without affecting or minimally affecting any of the other PD-1 interactions. In one aspect, the extent of binding of an anti-PD-1 antagonist antibody to an unrelated, non-PD-1 protein is less than about 10% of the binding of the antibody to PD-1 as measured, e.g., by a radioimmunoassay (RIA). In certain aspects, an anti-PD-1 antagonist antibody that binds to PD-1 has a dissociation constant (K_{D}) of ≤1 μM, ≤100 nM, ≤10 nM, ≤1 nM, ≤0.1 nM, ≤0.01 nM, or ≤0.001 nM (e.g., 10⁻⁸M or less, e.g., from 10⁻⁸M to 10⁻¹³M, e.g., from 10⁻⁹ M to 10⁻¹³ M). In certain aspects, an anti-PD-1 antagonist antibody binds to an epitope of PD-1 that is conserved among PD-1 from different species or an epitope on PD-1 that allows for cross-species reactivity. In one aspect, the anti-PD-1 antagonist antibody is pembrolizumab (previously known as lambrolizumab). In one aspect, the anti-PD-1 antagonist antibody is nivolumab.

The term “PD-L1 binding antagonist” refers to a molecule that decreases, blocks, inhibits, abrogates, or interferes with signal transduction resulting from the interaction of PD-L1 with either one or more of its binding partners, such as PD-1 and/or B7-1. In some instances, a PD-L1 binding antagonist is a molecule that inhibits the binding of PD-L1 to its binding partners. In a specific aspect, the PD-L1 binding antagonist inhibits binding of PD-L1 to PD-1 and/or B7-1. In some instances, the PD-L1 binding antagonists include anti-PD-L1 antibodies, antigen-binding fragments thereof, immunoadhesins, fusion proteins, oligopeptides and other molecules that decrease, block, inhibit, abrogate or interfere with signal transduction resulting from the interaction of PD-L1 with one or more of its binding partners, such as PD-1 and/or B7-1. In one instance, a PD-L1 binding antagonist reduces the negative co-stimulatory signal mediated by or through cell surface proteins expressed on T lymphocytes mediated signaling through PD-L1 so as to render a dysfunctional T-cell less dysfunctional (e.g., enhancing effector responses to antigen recognition). In some instances, the PD-L1 binding antagonist binds to PD-L1. In some instances, a PD-L1 binding antagonist is an anti-PD-L1 antibody (e.g., an anti-PD-L1 antagonist antibody). Exemplary anti-PD-L1 antagonist antibodies include atezolizumab, MDX-1105, MED14736 (durvalumab), MSB0010718C (avelumab), SHR-1316, CS1001, envafolimab, TQB2450, ZKAB001, LP-002, CX-072, IMC-001, KL-A167, APL-502, cosibelimab, lodapolimab, FAZ053, TG-1501, BGB-A333, BCD-135, AK-106, LDP, GR1405, HLX20, MSB2311, RC98, PDL-GEX, KD036, KY1003, YBL-007, and HS-636. In some aspects, the anti-PD-L1 antibody is atezolizumab, MDX-1105, MED14736 (durvalumab), or MSB0010718C (avelumab). In one specific aspect, the PD-L1 binding antagonist is MDX-1105. In another specific aspect, the PD-L1 binding antagonist is MED14736 (durvalumab). In another specific aspect, the PD-L1 binding antagonist is MSB0010718C (avelumab). In other aspects, the PD-L1 binding antagonist may be a small molecule, e.g., GS-4224, INCB086550, MAX-10181, INCB090244, CA-170, or ABSK041, which in some instances may be administered orally. Other exemplary PD-L1 binding antagonists include AVA-004, MT-6035, VXM10, LYN192, GB7003, and JS-003. In a preferred aspect, the PD-L1 binding antagonist is atezolizumab, marketed as TECENTRIQ™. Atezolizumab is described in WHO Drug Information (International Nonproprietary Names for Pharmaceutical Substances), Proposed INN: List 112, Vol. 28, No. 4, published Jan. 16, 2015 (see page 485). In another specific aspect, an anti PD-L1 antibody is MSB0015718C.

The term “anti-PD-L1 antagonist antibody” refers to an antibody or an antigen-binding fragment or variant thereof that is capable of binding PD-L1 with sufficient affinity such that it substantially or completely inhibits the biological activity of PD-L1. For example, an anti-PD-L1 antagonist antibody may decrease, block, inhibit, abrogate or interfere with signal transduction resulting from the interaction of PD-L1 with either one or more of its binding partners, such as PD-1 and/or B7-1. It will be understood by one of ordinary skill in the art that in some instances, an anti-PD-L1 antagonist antibody may antagonize one PD-L1 activity without affecting another PD-L1 activity. For example, an anti-PD-L1 antagonist antibody for use in certain of the methods or uses described herein is an anti-PD-L1 antagonist antibody that antagonizes PD-L1 activity in response to one of its binding partners (e.g., PD-1 or B7-1) without affecting or minimally affecting any of the other PD-L1 interactions. In one aspect, the extent of binding of an anti-PD-L1 antagonist antibody to an unrelated, non-PD-L1 protein is less than about 10% of the binding of the antibody to PD-L1 as measured, e.g., by a radioimmunoassay (RIA). In certain aspects, an anti-PD-L1 antagonist antibody that binds to PD-L1 has a dissociation constant (K_{D}) of ≤1 μM, ≤100 nM, ≤10 nM, ≤1 nM, ≤0.1 nM, ≤0.01 nM, or ≤0.001 nM (e.g., 10⁻⁸M or less, e.g., from 10⁻⁸M to 10⁻¹³M, e.g., from 10⁻⁹ M to 10⁻¹³ M). In certain aspects, an anti-PD-L1 antagonist antibody binds to an epitope of PD-L1 that is conserved among PD-L1 from different species or an epitope on PD-L1 that allows for cross-species reactivity. In one aspect, the anti-PD-L1 antagonist antibody is atezolizumab.

The term “PD-L2 binding antagonist” refers to a molecule that decreases, blocks, inhibits, abrogates or interferes with signal transduction resulting from the interaction of PD-L2 with either one or more of its binding partners, such as PD-1. PD-L2 (programmed death ligand 2) is also referred to in the art as “programmed cell death 1 ligand 2,” “PDCD1LG2,” “CD273,” “B7-DC,” “Btdc,” and “PDL2.” An exemplary human PD-L2 is shown in UniProtKB/Swiss-Prot Accession No. Q9BQ51. In some instances, a PD-L2 binding antagonist is a molecule that inhibits the binding of PD-L2 to one or more of its binding partners. In a specific aspect, the PD-L2 binding antagonist inhibits binding of PD-L2 to PD-1. Exemplary PD-L2 antagonists include anti-PD-L2 antibodies, antigen binding fragments thereof, immunoadhesins, fusion proteins, oligopeptides and other molecules that decrease, block, inhibit, abrogate or interfere with signal transduction resulting from the interaction of PD-L2 with either one or more of its binding partners, such as PD-1. In one aspect, a PD-L2 binding antagonist reduces the negative co-stimulatory signal mediated by or through cell surface proteins expressed on T lymphocytes mediated signaling through PD-L2 so as render a dysfunctional T-cell less dysfunctional (e.g., enhancing effector responses to antigen recognition). In some aspects, the PD-L2 binding antagonist binds to PD-L2. In some aspects, a PD-L2 binding antagonist is an immunoadhesin.

Further examples of PD-1 axis binding antagonists include cemiplimab, prolgolimab, camrelizumab, sintilimab, tislelizumab, toripalimab, dostarlimab, retifanlimab, spartalizumab, sasanlimab, penpulimab, CS1003, HLX10, SCT-110A, SHR-1316, CS1001, envafolimab, TQB2450, ZKAB001, LP-002, zimberelimab, balstilimab, genolimzumab, BI 754091, cetrelimab, YBL-006, BAT1306, HX008, CX-072, IMC-001, KL-A167, budigalimab, AMG 404, CX-188, JTX-4014, 609A, Sym021, LZM009, F520, SG001, APL-502, cosibelimab, lodapolimab, GS-4224, INCB086550, FAZ053, TG-1501, BGB-A333, BCD-135, AK-106, LDP, GR1405, HLX20, MSB2311, MAX-10181, RC98, BION-004, AM0001, CB201, ENUM 244C8, ENUM 388D4, AUNP-012, STI-1110, ADG104, AK-103, LBL-006, hAb21, AVA-004, PDL-GEX, INCB090244, KD036, KY1003, LYN192, MT-6035, VXM10, YBL-007, ABSK041, GB7003, JS-003, and HS-636.

For the purposes herein, “atezolizumab” is an Fc-engineered, humanized, non-glycosylated IgG1 kappa immunoglobulin that binds PD-L1 and comprises the heavy chain sequence of SEQ ID NO: 28 and the light chain sequence of SEQ ID NO: 29. Atezolizumab comprises a single amino acid substitution (asparagine to alanine) at position 297 on the heavy chain (N297A) using EU numbering of Fc region amino acid residues, which results in a non-glycosylated antibody that has minimal binding to Fc receptors. Atezolizumab is also described in WHO Drug Information (International Nonproprietary Names for Pharmaceutical Substances), Proposed INN: List 112, Vol. 28, No. 4, published Jan. 16, 2015 (see page 485).

As used herein, “pembrolizumab” is a recombinant humanized monoclonal IgG4 antibody directed against human cell surface receptor PD-1. Pembrolizumab is also described in WHO Drug Information (International Nonproprietary Names for Pharmaceutical Substances), Proposed INN: List 72, Vol. 28, No. 3, published 2014 (see page 407).

As used herein, “tiragolumab” is a fully human IgG1/kappa MAb-derived in Open Monoclonal Technology (OMT) rats that binds TIGIT and comprises the heavy chain sequence of SEQ ID NO: 33 and the light chain sequence of SEQ ID NO: 34. Tiragolumab comprises two N-linked glycosylation sites (N306) in the Fc domain. Tiragolumab is also described in WHO Drug Information (International Nonproprietary Names for Pharmaceutical Substances), Proposed INN: List 117, Vol. 31, No. 2, published Jul. 7, 2017 (see page 343).

As used herein, “bevacizumab” is a recombinant humanized monoclonal antibody that recognizes all isoforms of VEGF, which is described in WHO Drug Information (International Nonproprietary Names for Pharmaceutical Substances), Proposed INN: List 83, Vol. 14, No. 2, published 2000 (see page 107). Bevacizumab comprises the heavy chain variable region sequence of SEQ ID NO: X and the light chain variable region sequence of SEQ ID NO: X.

The term “anti-TIGIT antagonist antibody” refers to an antibody or an antigen-binding fragment or variant thereof that is capable of binding TIGIT with sufficient affinity such that it substantially or completely inhibits the biological activity of TIGIT. For example, an anti-TIGIT antagonist antibody may block signaling through PVR, PVRL2, and/or PVRL3 so as to restore a functional response by T-cells (e.g., proliferation, cytokine production, target cell killing) from a dysfunctional state to antigen stimulation. For example, an anti-TIGIT antagonist antibody may block signaling through PVR without impacting PVR-CD226 interaction. It will be understood by one of ordinary skill in the art that in some instances, an anti-TIGIT antagonist antibody may antagonize one TIGIT activity without affecting another TIGIT activity. For example, an anti-TIGIT antagonist antibody for use in certain of the methods or uses described herein is an anti-TIGIT antagonist antibody that antagonizes TIGIT activity in response to one of PVR interaction, PVRL3 interaction, or PVRL2 interaction, e.g., without affecting or minimally affecting any of the other TIGIT interactions. In one aspect, the extent of binding of an anti-TIGIT antagonist antibody to an unrelated, non-TIGIT protein is less than about 10% of the binding of the antibody to TIGIT as measured, e.g., by a radioimmunoassay (RIA). In certain aspects, an anti-TIGIT antagonist antibody that binds to TIGIT has a dissociation constant (K_{D}) of ≤1 μM, ≤100 nM, ≤10 nM, ≤1 nM, ≤0.1 nM, ≤0.01 nM, or ≤0.001 nM (e.g., 10⁻⁸M or less, e.g., from 10⁻⁸M to 10⁻¹³M, e.g., from 10⁻³M to 10⁻¹³ M). In certain aspects, an anti-TIGIT antagonist antibody binds to an epitope of TIGIT that is conserved among TIGIT from different species or an epitope on TIGIT that allows for cross-species reactivity. In some aspects, the anti-TIGIT binding antibody has intact Fc-mediated effector function (e.g., tiragolumab, vibostolimab, etigilimab, EOS084448, or TJ-T6). In some aspects, the anti-TIGIT binding antibody has enhanced Fc-mediated effector function (e.g., SGN-TGT). In other aspects, the anti-TIGIT binding antibody lacks Fc-mediated effector function (e.g., domvanalimab, BMS-986207, ASP8374, or COM902). In some aspects, the anti-TIGIT binding antibody is an IgG1 class antibody (e.g., tiragolumab, vibostolimab, domvanalimab, BMS-986207, etigilimab, BGB-A1217, SGN-TGT, EOS084448 (EOS-448), TJ-T6, or AB308). In other aspects, the anti-TIGIT binding antibody is an IgG4 class antibody (e.g., ASP8374 or COM902). In one aspect, the anti-TIGIT antagonist antibody is tiragolumab.

As used herein, “administering” is meant a method of giving a dosage of a compound (e.g., an anti-TIGIT antagonist antibody, a PD-1 axis binding antagonist (e.g., an anti-PD-L1 antibody), a VEGF antagonist, or a chemotherapeutic agent (e.g., a platinum-based chemotherapeutic agent (e.g., carboplatin or cisplatin) and/or one or more non-platinum-based chemotherapeutic agents (e.g., an alkylating agent (e.g., cyclophosphamide), a taxane (e.g., paclitaxel, e.g., nab-paclitaxel), and/or a topoisomerase II inhibitor (e.g., doxorubicin))) or a composition (e.g., a pharmaceutical composition, e.g., a pharmaceutical composition including an anti-TIGIT antagonist antibody, a PD-1 axis binding antagonist (e.g., an anti-PD-L1 antibody), a chemotherapeutic agent (e.g., a platinum-based chemotherapeutic agent (e.g., carboplatin or cisplatin) and/or one or more non-platinum-based chemotherapeutic agents (e.g., an alkylating agent (e.g., cyclophosphamide), a taxane (e.g., paclitaxel, e.g., nab-paclitaxel), and/or a topoisomerase II inhibitor (e.g., doxorubicin)), an antibody-drug conjugate (ADC) (e.g., enfortumab vedotin or sacituzumab govitecan), and/or a colony stimulating factor (CSF) (e.g., pegfilgrastim, filgrastim, or sargramostim)) to a subject. The compounds and/or compositions utilized in the methods described herein can be administered, for example, intravenously (e.g., by intravenous infusion), subcutaneously, intramuscularly, intradermally, percutaneously, intraarterially, intraperitoneally, intralesionally, intracranially, intraarticularly, intraprostatically, intrapleurally, intratracheally, intranasally, intravitreally, intravaginally, intrarectally, topically, intratumorally, peritoneally, subconjunctivally, intravesicularlly, mucosally, intrapericardially, intraumbilically, intraocularly, orally, topically, locally, by inhalation, by injection, by infusion, by continuous infusion, by localized perfusion bathing target cells directly, by catheter, by lavage, in cremes, or in lipid compositions. The method of administration can vary depending on various factors (e.g., the compound or composition being administered and the severity of the condition, disease, or disorder being treated).

A “fixed” or “flat” dose of a therapeutic agent (e.g., an anti-TIGIT antagonist antibody, a PD-1 axis binding antagonist (e.g., an anti-PD-L1 antibody), a VEGF antagonist, a chemotherapeutic agent (e.g., a platinum-based chemotherapeutic agent or non-platinum-based chemotherapeutic agent), an ADC (e.g., enfortumab vedotin or sacituzumab govitecan), or a CSF (e.g., pegfilgrastim, filgrastim, or sargramostim)) refers to a dose that is administered to a patient without regard for the weight or body surface area (BSA) of the patient. The fixed or flat dose is therefore not provided as a mg/kg dose or a mg/m² dose, but rather as an absolute amount of the therapeutic agent (e.g., absolute amount of the therapeutic agent in mg).

As used herein, the term “treatment” or “treating” refers to clinical intervention designed to alter the natural course of the individual or cell being treated during the course of clinical pathology. Desirable effects of treatment include delaying or decreasing the rate of disease progression, ameliorating or palliating the disease state, and remission or improved prognosis. For example, an individual is successfully “treated” if one or more symptoms associated with cancer are mitigated or eliminated, including, but are not limited to, reducing the proliferation of (or destroying) cancerous cells, decreasing symptoms resulting from the disease, increasing the quality of life of those suffering from the disease, decreasing the dose of other medications required to treat the disease, delaying the progression of the disease, and/or prolonging survival of individuals. For example, treating comprises effective cancer treatment with an effective amount of a therapeutic agent (e.g., an anti-TIGIT antagonist antibody, a PD-1 axis binding antagonist, a VEGF antagonist, a chemotherapeutic agent (e.g., a platinum-based chemotherapeutic agent or non-platinum-based chemotherapeutic agent), an ADC (e.g., enfortumab vedotin or sacituzumab govitecan), and/or a CSF (e.g., pegfilgrastim, filgrastim, or sargramostim)) or combination of therapeutic agents. Treating herein includes, inter alia, adjuvant therapy, neoadjuvant therapy, non-metastatic cancer therapy (e.g., locally advanced cancer therapy), and metastatic cancer therapy. The treatment may be first-line treatment (e.g., the patient may be previously untreated or not have received prior systemic therapy), or second line or later treatment.

As used herein, “in combination with” or “in conjunction with” refers to administration of one treatment modality in addition to another treatment modality, for example, a treatment regimen that includes administration of a PD-1 axis binding antagonist (e.g., atezolizumab), a VEGF antagonist, a chemotherapeutic agent (e.g., a platinum-based chemotherapeutic agent or non-platinum-based chemotherapeutic agent), an ADC (e.g., enfortumab vedotin or sacituzumab govitecan), and/or a CSF (e.g., pegfilgrastim, filgrastim, or sargramostim) and an anti-TIGIT antagonist antibody (e.g., an anti-TIGIT antagonist antibody as disclosed herein, e.g., tiragolumab). As such, “in combination with” refers to administration of one treatment modality before, during, or after administration of the other treatment modality to the patient.

A drug that is administered “concurrently” with one or more other drugs is administered during the same treatment cycle, on the same day of treatment, as the one or more other drugs, and, optionally, at the same time as the one or more other drugs. For instance, for cancer therapies given every 3 weeks, the concurrently administered drugs are each administered on day 1 of a 3-week cycle.

As used herein, the term “perioperative treatment” refers to a treatment that is administered before and after a surgery. A perioperative treatment may include administration of a neoadjuvant therapy prior to a surgery (e.g., a cystectomy) and a therapy (e.g., an adjuvant therapy) following the surgery. For example, a perioperative treatment may include a neoadjuvant therapy (e.g., an anti-TIGIT antagonist antibody and a PD-1 axis binding antagonist neoadjuvant therapy) that is administered after diagnosis and before (e.g., about 1, 2, 3, 4, 5, 6, 7, 8, 9, 12, 13, 14, 15 weeks or more before) a surgery (e.g., a cystectomy) and a therapy (e.g., an adjuvant therapy (e.g., an anti-TIGIT antagonist antibody and a PD-1 axis binding antagonist adjuvant therapy)) following the surgery (e.g., about 1, 2, 3, 4, 5, 6, 7, 8, 9, 12, 13, 14, 15 weeks or more following the surgery).

A “disorder” or “disease” is any condition that would benefit from treatment including, but not limited to, disorders that are associated with some degree of abnormal cell proliferation, e.g., cancer.

The term “dysfunction,” in the context of immune dysfunction, refers to a state of reduced immune responsiveness to antigenic stimulation.

The term “dysfunctional,” as used herein, also includes refractory or unresponsive to antigen recognition, specifically, impaired capacity to translate antigen recognition into downstream T-cell effector functions, such as proliferation, cytokine production (e.g., gamma interferon) and/or target cell killing.

The terms “cancer” and “cancerous” refer to or describe the physiological condition in mammals that is typically characterized by unregulated cell growth. Cancers include solid tumor cancers and non-solid tumor cancers and locally advanced or metastatic cancers (e.g., locally advanced or metastatic tumors). Examples of cancer include but are not limited to, carcinoma, lymphoma, blastoma, sarcoma, and leukemia or lymphoid malignancies. More particular examples of such cancers include, but are not limited to urothelial carcinoma (UC), including locally advanced and metastatic UC (mUC), bladder cancer (e.g., muscle invasive bladder cancer (MIBC) and non-muscle invasive bladder cancer (NMIBC), e.g., BCG-refractory NMIBC), MIBC urothelial bladder cancer (UBC); kidney or renal cancer (e.g., renal cell carcinoma (RCC)); cancer of the urinary tract; lung cancer, such as small cell lung cancer (SCLC), which includes extensive stage SCLC (ES-SCLC); non-small cell lung cancer (NSCLC), which includes squamous NSCLC or non-squamous NSCLC, including locally advanced unresectable NSCLC (e.g., Stage IIIB NSCLC), or recurrent or metastatic NSCLC (e.g., Stage IV NSCLC), adenocarcinoma of the lung, or squamous cell cancer (e.g., epithelial squamous cell cancer (e.g., squamous carcinoma of the lung); pancreatic cancer (e.g., pancreatic ductal adenocarcinoma (PDAC), e.g., metastatic PDAC)); head and neck cancer (e.g., SCCHN, e.g., recurrent/metastatic PD-L1-positive SCCHN, and head and neck squamous cell cancer (HNSCC); ovarian cancer (OC); esophageal cancer; cancer of the peritoneum; hepatocellular cancer; gastric cancer (GC) (e.g., gastroesophageal junction (GEJ) cancer) or stomach cancer, including gastrointestinal cancer and gastrointestinal stromal cancer; glioblastoma; cancer of the urinary tract; hepatoma; breast cancer (e.g., HER2+ breast cancer and triple-negative breast cancer (TNBC (e.g., early TNBC (eTNBC)), which are estrogen receptors (ER−), progesterone receptors (PgR−), and HER2 (HER2−) negative); prostate cancer, such as castration-resistant prostate cancer (CRPC); cancer of the peritoneum; hepatocellular cancer; gastric or stomach cancer, including gastrointestinal cancer and gastrointestinal stromal cancer; pancreatic cancer (e.g., pancreatic ductal adenocarcinoma (PDAC)); glioblastoma; cervical cancer (e.g., a Stage IVB, metastatic, recurrent, or persistent cervical cancer, e.g., a metastatic and/or recurrent PD-L1-positive cervical carcinoma); ovarian cancer; liver cancer (e.g., hepatocellular carcinoma (HCC), e.g., locally advanced or metastatic HCC and/or unresectable HCC); hepatoma; colon cancer; rectal cancer; colorectal cancer (CRC; e.g., CRC with microsatellite-stable (MSS) and microsatellite instability (MSI) low (MSI-Low)); endometrial or uterine carcinoma; salivary gland carcinoma; prostate cancer; vulval cancer; thyroid cancer; hepatic carcinoma; anal carcinoma; penile carcinoma; melanoma, including superficial spreading melanoma, lentigo maligna melanoma, acral lentiginous melanomas, and nodular melanomas; multiple myeloma and B-cell lymphoma (including low grade/follicular non-Hodgkin's lymphoma (NHL)); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia); chronic lymphocytic leukemia (CLL); acute lymphoblastic leukemia (ALL); acute myologenous leukemia (AML); hairy cell leukemia; chronic myeloblastic leukemia (CML); post-transplant lymphoproliferative disorder (PTLD); and myelodysplastic syndromes (MDS), as well as abnormal vascular proliferation associated with phakomatoses, edema (such as that associated with brain tumors), Meigs' syndrome, brain cancer, head and neck cancer, and associated metastases.

The term “persistent cervical cancer” as used herein refers to a cervical cancer that has not been rendered undetectable or benign after previous therapy.

As used herein, “Stage IVB cervical cancer” refers to a cervical cancer that is classified as such using a cervical cancer staging system (e.g., International Federation of Gynecology and Obstetrics (FIGO) staging system). In some aspects, a cervical cancer is classified as Stage IVB if it has metastasized to distant organs (including the parenchyma of the spleen or liver) or to the inguinal and extra-abdominal lymph nodes.

As used herein, the term “recurrent cervical cancer” refers to a cervical cancer that has been detected or has returned following an initial treatment with surgery, radiation therapy, and/or chemotherapy.

The term “TNBC” refers to breast cancer that lacks expression of ER, PR, and HER2. The term “eTNBC” refers to T2-4d TNBC (e.g., cT2-cT4, cN0-cN3, and cM0).

Head and neck cancers include cancers that begin in the mucosal surfaces of the upper aerodigestive tract and affect the oral cavity, oropharynx, larynx, hypopharynx, and nasopharynx.

As used herein, “urothelial carcinoma” and “UC” refer to a type of cancer that typically occurs in the urinary system, and includes muscle-invasive bladder cancer (MIBC) and muscle-invasive urinary tract urothelial cancer (UTUC). UC is also referred to in the art as transitional cell carcinoma (TCC).

The term “tumor” refers to all neoplastic cell growth and proliferation, whether malignant or benign, and all pre-cancerous and cancerous cells and tissues. The terms “cancer,” “cancerous,” “cell proliferative disorder,” “proliferative disorder,” and “tumor” are not mutually exclusive as referred to herein.

A “tumor cell” as used herein, refers to any tumor cell present in a tumor or a sample thereof. Tumor cells may be distinguished from other cells that may be present in a tumor sample, for example, stromal cells and tumor-infiltrating immune cells, using methods known in the art and/or described herein.

“Tumor immunity” refers to the process in which tumors evade immune recognition and clearance. Thus, as a therapeutic concept, tumor immunity is “treated” when such evasion is attenuated, and the tumors are recognized and attacked by the immune system. Examples of tumor recognition include tumor binding, tumor shrinkage, and tumor clearance.

As used herein, “metastasis” is meant the spread of cancer from its primary site to other places in the body. Cancer cells can break away from a primary tumor, penetrate into lymphatic and blood vessels, circulate through the bloodstream, and grow in a distant focus (metastasize) in normal tissues elsewhere in the body. Metastasis can be local or distant. Metastasis is a sequential process, contingent on tumor cells breaking off from the primary tumor, traveling through the bloodstream, and stopping at a distant site. At the new site, the cells establish a blood supply and can grow to form a life-threatening mass. Both stimulatory and inhibitory molecular pathways within the tumor cell regulate this behavior, and interactions between the tumor cell and host cells in the distant site are also significant.

The term “cytotoxic agent” as used herein refers to any agent that is detrimental to cells (e.g., causes cell death or destruction, inhibits proliferation, or otherwise inhibits or prevents a cellular function). Cytotoxic agents include, but are not limited to, radioactive isotopes (e.g., At²¹¹, I¹³¹, I¹²⁵, Y⁹⁰, Re¹⁸⁶, Re¹⁸⁸, Sm¹⁵³, Bi²¹², P³², Pb²¹² and radioactive isotopes of Lu); chemotherapeutic agents or drugs (e.g., methotrexate, adriamicin, vinca alkaloids (vincristine, vinblastine, etoposide), doxorubicin, melphalan, mitomycin C, chlorambucil, daunorubicin or other intercalating agents); growth inhibitory agents; enzymes and fragments thereof such as nucleolytic enzymes; antibiotics; and toxins such as small molecule toxins or enzymatically active toxins of bacterial, fungal, plant or animal origin, including fragments and/or variants thereof; and the various antitumor or anti-cancer agents disclosed below. Exemplary cytotoxic agents can be selected from anti-microtubule agents, platinum coordination complexes, alkylating agents, topoisomerase II inhibitors, antimetabolites, topoisomerase I inhibitors, hormones and hormonal analogues, signal transduction pathway inhibitors, non-receptor tyrosine kinase angiogenesis inhibitors, immunotherapeutic agents, proapoptotic agents, inhibitors of LDH-A, inhibitors of fatty acid biosynthesis, cell cycle signaling inhibitors, HDAC inhibitors, proteasome inhibitors, and inhibitors of cancer metabolism. In one instance, the cytotoxic agent is a platinum-based chemotherapeutic agent (e.g., carboplatin or cisplatin). In one instance, the cytotoxic agent is an antagonist of EGFR, e.g., N-(3-ethynylphenyl)-6,7-bis(2-methoxyethoxy)quinazolin-4-amine (e.g., erlotinib). In one instance the cytotoxic agent is a RAF inhibitor, e.g., a BRAF and/or CRAF inhibitor. In one instance the RAF inhibitor is vemurafenib. In one instance, the cytotoxic agent is a PI3K inhibitor.

“Chemotherapeutic agent” includes chemical compounds useful in the treatment of cancer. Examples of chemotherapeutic agents include erlotinib (TARCEVA®, Genentech/OSI Pharm.), bortezomib (VELCADE®, Millennium Pharm.), disulfiram, epigallocatechin gallate, salinosporamide A, carfilzomib, 17-AAG (geldanamycin), radicicol, lactate dehydrogenase A (LDH-A), fulvestrant (FASLODEX®, AstraZeneca), sunitinib (SUTENT®, Pfizer/Sugen), letrozole (FEMARA®, Novartis), imatinib mesylate (GLEEVEC®, Novartis), finasunate (VATALANIB®, Novartis), oxaliplatin (ELOXATIN®, Sanofi), 5-FU (5-fluorouracil), leucovorin, Rapamycin (Sirolimus, RAPAMUNE®, Wyeth), Lapatinib (TYKERB®, GSK572016, Glaxo Smith Kline), Lonafamib (SCH 66336), sorafenib (NEXAVAR®, Bayer Labs), gefitinib (IRESSA®, AstraZeneca), AG1478, alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylomelamine; acetogenins (especially bullatacin and bullatacinone); a camptothecin (including topotecan and irinotecan); bryostatin; callystatin; CC-1065 (including its adozelesin, carzelesin and bizelesin synthetic analogs); cryptophycins (particularly cryptophycin 1 and cryptophycin 8); adrenocorticosteroids (including prednisone and prednisolone); cyproterone acetate; 5α-reductases including finasteride and dutasteride); vorinostat, romidepsin, panobinostat, valproic acid, mocetinostat dolastatin; aldesleukin, talc duocarmycin (including the synthetic analogs, KW-2189 and CB1-TM1); eleutherobin; pancratistatin; a sarcodictyin; spongistatin; nitrogen mustards such as chlorambucil, chlomaphazine, chlorophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, fotemustine, lomustine, nimustine, and ranimnustine; antibiotics such as the enediyne antibiotics (e.g., calicheamicin, especially calicheamicin γ1I and calicheamicin ω1I (Angew Chem. Intl. Ed. Engl. 1994 33:183-186); dynemicin, including dynemicin A; bisphosphonates, such as clodronate; an esperamicin; as well as neocarzinostatin chromophore and related chromoprotein enediyne antibiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN® (doxorubicin), morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogs such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfomithine; elliptinium acetate; an epothilone; etoglucid; gallium nitrate; hydroxyurea; lentinan; lonidainine; maytansinoids such as maytansine and ansamitocins; mitoguazone; mitoxantrone; mopidamnol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxanes (taxoids), e.g., TAXOL® (paclitaxel; Bristol-Myers Squibb Oncology, Princeton, N.J.), ABRAXANE® (Cremophor-free), albumin-engineered nanoparticle formulations of paclitaxel (e.g., nanoparticle albumin-engineered paclitaxel (nab-paclitaxel)) (American Pharmaceutical Partners, Schaumberg, Ill.), and TAXOTERE® (docetaxel, doxetaxel; Sanofi-Aventis); chloranmbucil; GEMZAR® (gemcitabine); 6-thioguanine; mercaptopurine; methotrexate; platinum analogs such as cisplatin and carboplatin; vinblastine; etoposide (VP-16); ifosfamide; mitoxantrone; vincristine; NAVELBINE® (vinorelbine); novantrone; teniposide; edatrexate; daunomycin; aminopterin; capecitabine (XELODA®); ibandronate; CPT-11; topoisomerase inhibitor RFS 2000; difluoromethylornithine (DMFO); retinoids such as retinoic acid; and pharmaceutically acceptable salts, acids and derivatives of any of the above.

Chemotherapeutic agents also include (i) anti-hormonal agents that act to regulate or inhibit hormone action on tumors such as anti-estrogens and selective estrogen receptor modulators (SERMs), including, for example, tamoxifen (including NOLVADEX®; tamoxifen citrate), raloxifene, droloxifene, iodoxyfene, 4-hydroxytamoxifen, trioxifene, keoxifene, LY117018, onapristone, and FARESTON® (toremifine citrate); (ii) aromatase inhibitors that inhibit the enzyme aromatase, which regulates estrogen production in the adrenal glands, such as, for example, 4(5)-imidazoles, aminoglutethimide, MEGASE® (megestrol acetate), AROMASIN® (exemestane; Pfizer), formestanie, fadrozole, RIVISOR® (vorozole), FEMARA® (letrozole; Novartis), and ARIMIDEX® (anastrozole; AstraZeneca); (iii) anti-androgens such as flutamide, nilutamide, bicalutamide, leuprolide and goserelin; buserelin, tripterelin, medroxyprogesterone acetate, diethylstilbestrol, premarin, fluoxymesterone, all trans retionic acid, fenretinide, as well as troxacitabine (a 1,3-dioxolane nucleoside cytosine analog); (iv) protein kinase inhibitors (e.g., an anaplastic lymphoma kinase (Alk) inhibitor, such as AF-802 (also known as CH-5424802 or alectinib)); (v) lipid kinase inhibitors; (vi) antisense oligonucleotides, particularly those which inhibit expression of genes in signaling pathways implicated in aberrant cell proliferation, such as, for example, PKC-alpha, Ralf and H-Ras; (vii) ribozymes such as VEGF expression inhibitors (e.g., ANGIOZYME®) and HER2 expression inhibitors; (viii) vaccines such as gene therapy vaccines, for example, ALLOVECTIN®, LEUVECTIN®, and VAXID®; PROLEUKIN®, rIL-2; a topoisomerase 1 inhibitor such as LURTOTECAN®; ABARELIX® rmRH; and (ix) pharmaceutically acceptable salts, acids and derivatives of any of the above.

Chemotherapeutic agents also include “platinum-based” chemotherapeutic agents, also referred to herein as “platinum agents,” which comprise an organic compound which contains platinum as an integral part of the molecule. Typically, platinum-based chemotherapeutic agents are coordination complexes of platinum. Platinum-based chemotherapeutic agents are sometimes called “platins” in the art. Examples of platinum-based chemotherapeutic agents include, but are not limited to, cisplatin, carboplatin, oxaliplatin, nedaplatin, triplatin tetranitrate, phenanthriplatin, picoplatin, lipoplatin, and satraplatin. Platinum-based chemotherapeutic agents (e.g., cisplatin or carboplatin) may be administered in combination with one or more additional chemotherapeutic agents, e.g., a nucleoside analog (e.g., gemcitabine), an antimetabolite (e.g., pemetrexed or gemcitabine), or a taxane (e.g., paclitaxel or nab-paclitaxel).

The term “eligible for treatment with a platinum-based chemotherapy” means that the subject is eligible for treatment with a platinum-based chemotherapy, either in the attending clinician's judgment or according to standardized criteria for eligibility for platinum-based chemotherapy that are known in the art.

Chemotherapeutic agents also include “non-platinum-based chemotherapeutic agents,” which, as used herein, refer to chemotherapeutic agents that are not “platinum-based.” As used herein, the terms “non-platinum-based chemotherapeutic agents” and “non-platinum agents” are used interchangeably. Exemplary non-platinum-based chemotherapeutic agents include antimetabolites (e.g., pemetrexed and gemcitabine), topoisomerase II inhibitors (e.g., etoposide, teniposide, doxorubicin, daunorubicin, mitoxantrone, amsacrine, an ellipticine, aurintricarboxylic acid, or HU-331), taxanes (e.g., paclitaxel (e.g., albumin-engineered paclitaxel, also referred to as nanoparticle-albumin-bound paclitaxel (nab-paclitaxel)), docetaxel, larotaxel, cabazitaxel, milataxel, tesetaxel, and/or orataxel). Exemplary non-platinum-based chemotherapeutic agents also include alkylating agents (e.g., cyclophosphamide).

A “nucleoside analog,” as used herein, refers to a nucleoside that includes a nucleic acid analog and a sugar. Nucleoside analogs may function as antimetabolites. Exemplary nucleoside analogues include but are not limited to gemcitabine, cytarabine, fludarabine, and cladribine.

A “taxane” as used herein is a diterpene which may bind to tubulin, promoting microtubule assembly and stabilization and/or prevent microtubule depolymerization. Taxanes included herein include taxoid 10-deacetylbaccatin III and/or derivatives thereof. Exemplary taxanes include, but are not limited to, paclitaxel (i.e., TAXOL®, CAS #33069-62-4), docetaxel (i.e., TAXOTERE®, CAS #114977-28-5), larotaxel, cabazitaxel, milataxel, tesetaxel, and/or orataxel. In some aspects, the taxane is an albumin-coated nanoparticle (e.g., nab-paclitaxel, i.e., ABRAXANE® and/or nab-docetaxel, ABI-008). In some aspects, the taxane is nab-paclitaxel (ABRAXANE®). In some aspects, the taxane is formulated in CREMAPHOR® (e.g., TAXOL®) and/or in Tween such as polysorbate 80 (e.g., TAXOTERE®). In some aspects, the taxane is liposome-encapsulated taxane. In some aspects, the taxane is a prodrug form and/or conjugated form of taxane (e.g., DHA covalently conjugated to paclitaxel, paclitaxel poliglumex, and/or linoleyl carbonate-paclitaxel). In some aspects, the paclitaxel is formulated with substantially no surfactant (e.g., in the absence of CREMAPHOR and/or Tween-such as TOCOSOL® paclitaxel).

An “antimetabolite” as used herein is a chemotherapeutic agent that interferes with and inhibits (wholly or partially) an endogenous (normal) metabolic process within a cell (e.g., a cancer cell). Antimetabolites include gemcitabine, pemetrexed, capecitabine, hydroxyurea, methotrexate, fluorouracil, cladribine, mercaptopurine, and pralatrexate.

Chemotherapeutic agents also include dexamethasone, interferons, colchicine, metoprine, cyclosporine, amphotericin, metronidazole, alemtuzumab, alitretinoin, allopurinol, amifostine, arsenic trioxide, asparaginase, BCG live, bevacizumab, bexarotene, cladribine, clofarabine, darbepoetin alfa, denileukin, dexrazoxane, epoetin alfa, elotinib, filgrastim, histrelin acetate, ibritumomab, interferon alfa-2a, interferon alfa-2b, lenalidomide, levamisole, mesna, methoxsalen, nandrolone, nelarabine, nofetumomab, oprelvekin, palifermin, pamidronate, pegademase, pegaspargase, pegfilgrastim, pemetrexed disodium, plicamycin, porfimer sodium, quinacrine, rasburicase, sargramostim, temozolomide, VM-26, 6-TG, toremifene, tretinoin, ATRA, valrubicin, zoledronate, and zoledronic acid, and pharmaceutically acceptable salts thereof.

Chemotherapeutic agents also include hydrocortisone, hydrocortisone acetate, cortisone acetate, tixocortol pivalate, triamcinolone acetonide, triamcinolone alcohol, mometasone, amcinonide, budesonide, desonide, fluocinonide, fluocinolone acetonide, betamethasone, betamethasone sodium phosphate, dexamethasone, dexamethasone sodium phosphate, fluocortolone, hydrocortisone-17-butyrate, hydrocortisone-17-valerate, aclometasone dipropionate, betamethasone valerate, betamethasone dipropionate, prednicarbate, clobetasone-17-butyrate, clobetasol-17-propionate, fluocortolone caproate, fluocortolone pivalate and fluprednidene acetate; immune selective anti-inflammatory peptides (ImSAIDs) such as phenylalanine-glutamine-glycine (FEG) and its D-isomeric form (feG) (IMULAN BioTherapeutics, LLC); anti-rheumatic drugs such as azathioprine, ciclosporin (cyclosporine A), D-penicillamine, gold salts, hydroxychloroquine, leflunomideminocycline, sulfasalazine, tumor necrosis factor alpha (TNFα) blockers such as etanercept (Enbrel), infliximab (Remicade), adalimumab (Humira), certolizumab pegol (Cimzia), golimumab (Simponi), Interleukin 1 (IL-1) blockers such as anakinra (Kineret), T cell costimulation blockers such as abatacept (Orencia), Interleukin 6 (IL-6) blockers such as tocilizumab (ACTEMERA®); Interleukin 13 (IL-13) blockers such as lebrikizumab; Interferon alpha (IFN) blockers such as Rontalizumab; Beta 7 integrin blockers such as rhuMAb Beta7; IgE pathway blockers such as Anti-M1 prime; Secreted homotrimeric LTa3 and membrane bound heterotrimer LTa1/β2 blockers such as Anti-lymphotoxin alpha (LTa); radioactive isotopes (e.g., At211, I131, I125, Y90, Re186, Re188, Sm153, Bi212, P32, Pb212 and radioactive isotopes of Lu); miscellaneous investigational agents such as thioplatin, PS-341, phenylbutyrate, ET-18-OCH3, or farnesyl transferase inhibitors (L-739749, L-744832); polyphenols such as quercetin, resveratrol, piceatannol, epigallocatechine gallate, theaflavins, flavanols, procyanidins, betulinic acid and derivatives thereof; autophagy inhibitors such as chloroquine; delta-9-tetrahydrocannabinol (dronabinol, MARINOL®); beta-lapachone; lapachol; colchicines; betulinic acid; acetylcamptothecin, scopolectin, and 9-aminocamptothecin); podophyllotoxin; tegafur (UFTORAL®); bexarotene (TARGRETIN®); bisphosphonates such as clodronate (for example, BONEFOS® or OSTAC®), etidronate (DIDROCAL®), NE-58095, zoledronic acid/zoledronate (ZOMETA®), alendronate (FOSAMAX®), pamidronate (AREDIA®), tiludronate (SKELID®), or risedronate (ACTONEL®); and epidermal growth factor receptor (EGF-R); vaccines such as THERATOPE® vaccine; perifosine, COX-2 inhibitor (e.g., celecoxib or etoricoxib), proteosome inhibitor (e.g., PS341); CCI-779; tipifarnib (R11577); orafenib, ABT510; Bcl-2 inhibitor such as oblimersen sodium (GENASENSE®); pixantrone; farnesyltransferase inhibitors such as lonafarnib (SCH 6636, SARASAR™); and pharmaceutically acceptable salts, acids or derivatives of any of the above; as well as combinations of two or more of the above such as CHOP, an abbreviation for a combined therapy of cyclophosphamide, doxorubicin, vincristine, and prednisolone; and FOLFOX, an abbreviation for a treatment regimen with oxaliplatin (ELOXATIN™) combined with 5-FU and leucovorin.

Chemotherapeutic agents also include non-steroidal anti-inflammatory drugs with analgesic, antipyretic and anti-inflammatory effects. NSAIDs include non-selective inhibitors of the enzyme cyclooxygenase. Specific examples of NSAIDs include aspirin, propionic acid derivatives such as ibuprofen, fenoprofen, ketoprofen, flurbiprofen, oxaprozin and naproxen, acetic acid derivatives such as indomethacin, sulindac, etodolac, diclofenac, enolic acid derivatives such as piroxicam, meloxicam, tenoxicam, droxicam, lornoxicam and isoxicam, fenamic acid derivatives such as mefenamic acid, meclofenamic acid, flufenamic acid, tolfenamic acid, and COX-2 inhibitors such as celecoxib, etoricoxib, lumiracoxib, parecoxib, rofecoxib, and valdecoxib. NSAIDs can be indicated for the symptomatic relief of conditions such as rheumatoid arthritis, osteoarthritis, inflammatory arthropathies, ankylosing spondylitis, psoriatic arthritis, Reiter's syndrome, acute gout, dysmenorrhoea, metastatic bone pain, headache and migraine, postoperative pain, mild-to-moderate pain due to inflammation and tissue injury, pyrexia, ileus, and renal colic.

Chemotherapeutic agents also include “EGFR inhibitors,” which refers to compounds that bind to or otherwise interact directly with EGFR and prevent or reduce its signaling activity, and is alternatively referred to as an “EGFR antagonist.” Examples of such agents include small molecules that bind to EGFR. EGFR antagonists include small molecules such as compounds described in U.S. Pat. Nos. 5,616,582, 5,457,105, 5,475,001, 5,654,307, 5,679,683, 6,084,095, 6,265,410, 6,455,534, 6,521,620, 6,596,726, 6,713,484, 5,770,599, 6,140,332, 5,866,572, 6,399,602, 6,344,459, 6,602,863, 6,391,874, 6,344,455, 5,760,041, 6,002,008, and 5,747,498, as well as the following PCT publications: WO98/14451, WO98/50038, WO99/09016, and WO99/24037. Particular small molecule EGFR antagonists include OSI-774 (CP-358774, erlotinib, TARCEVA® Genentech/OSI Pharmaceuticals); PD 183805 (CI 1033, 2-propenamide, N-[4-[(3-chloro-4-fluorophenyl)amino]-7-[3-(4-morpholinyl)propoxy]-6-quinazolinyl]-, dihydrochloride, Pfizer Inc.); ZD1839, gefitinib (IRESSA®) 4-(3′-Chloro-4′-fluoroanilino)-7-methoxy-6-(3-morpholinopropoxy)quinazoline, AstraZeneca); ZM 105180 ((6-amino-4-(3-methylphenyl-amino)-quinazoline, Zeneca); BIBX-1382 (N8-(3-chloro-4-fluoro-phenyl)-N2-(1-methyl-piperidin-4-yl)-pyrimido[5,4-d]pyrimidine-2,8-diamine, Boehringer Ingelheim); PKI-166 ((R)-4-[4-[(1-phenylethyl)amino]-1H-pyrrolo[2,3-d]pyrimidin-6-yl]-phenol); (R)-6-(4-hydroxyphenyl)-4-[(1-phenylethyl)amino]-7H-pyrrolo[2,3-d]pyrimidine); CL-387785 (N-[4-[(3-bromophenyl)amino]-6-quinazolinyl]-2-butynamide); EKB-569 (N-[4-[(3-chloro-4-fluorophenyl)amino]-3-cyano-7-ethoxy-6-quinolinyl]-4-(dimethylamino)-2-butenamide) (Wyeth); AG1478 (Pfizer); AG1571 (SU 5271; Pfizer); dual EGFR/HER2 tyrosine kinase inhibitors such as lapatinib (TYKERB®, GSK572016 or N-[3-chloro-4-[(3 fluorophenyOmethoxy]phenyl]-6[5[[[2methylsulfonyl)ethyl]amino]methyl]-2-furanyl]-4-quinazolinamine).

Chemotherapeutic agents also include “tyrosine kinase inhibitors” including the EGFR-targeted drugs noted in the preceding paragraph; inhibitors of insulin receptor tyrosine kinases, including anaplastic lymphoma kinase (Alk) inhibitors, such as AF-802 (also known as CH-5424802 or alectinib), ASP3026, X396, LDK378, AP26113, crizotinib (XALKORI®), and ceritinib (ZYKADIA®); small molecule HER2 tyrosine kinase inhibitor such as TAK165 available from Takeda; CP-724,714, an oral selective inhibitor of the ErbB2 receptor tyrosine kinase (Pfizer and OSI); dual-HER inhibitors such as EKB-569 (available from Wyeth) which preferentially binds EGFR but inhibits both HER2 and EGFR-overexpressing cells; lapatinib (GSK572016; available from Glaxo-SmithKline), an oral HER2 and EGFR tyrosine kinase inhibitor; PKI-166 (available from Novartis); pan-HER inhibitors such as canertinib (CI-1033; Pharmacia); Raf-1 inhibitors such as antisense agent ISIS-5132 available from ISIS Pharmaceuticals which inhibit Raf-1 signaling; non-HER targeted TK inhibitors such as imatinib mesylate (GLEEVEC®, available from Glaxo SmithKline); multi-targeted tyrosine kinase inhibitors such as sunitinib (SUTENT®, available from Pfizer); VEGF receptor tyrosine kinase inhibitors such as vatalanib (PTK787/ZK222584, available from Novartis/Schering AG); MAPK extracellular regulated kinase I inhibitor CI-1040 (available from Pharmacia); quinazolines, such as PD 153035,4-(3-chloroanilino) quinazoline; pyridopyrimidines; pyrimidopyrimidines; pyrrolopyrimidines, such as CGP 59326, CGP 60261 and CGP 62706; pyrazolopyrimidines, 4-(phenylamino)-7H-pyrrolo[2,3-d] pyrimidines; curcumin (diferuloyl methane, 4,5-bis (4-fluoroanilino)phthalimide); tyrphostines containing nitrothiophene moieties; PD-0183805 (Warner-Lamber); antisense molecules (e.g., those that bind to HER-encoding nucleic acid); quinoxalines (U.S. Pat. No. 5,804,396); tryphostins (U.S. Pat. No. 5,804,396); ZD6474 (Astra Zeneca); PTK-787 (Novartis/Schering AG); pan-HER inhibitors such as CI-1033 (Pfizer); Affinitac (ISIS 3521; Isis/Lilly); imatinib mesylate (GLEEVEC®); PKI 166 (Novartis); GW2016 (Glaxo SmithKline); CI-1033 (Pfizer); EKB-569 (Wyeth); Semaxinib (Pfizer); ZD6474 (AstraZeneca); PTK-787 (Novartis/Schering AG); INC-1C11 (Imclone), rapamycin (sirolimus, RAPAMUNE®); or as described in any of the following patent publications: U.S. Pat. No. 5,804,396; WO 1999/09016 (American Cyanamid); WO 1998/43960 (American Cyanamid); WO 1997/38983 (Warner Lambert); WO 1999/06378 (Warner Lambert); WO 1999/06396 (Warner Lambert); WO 1996/30347 (Pfizer, Inc); WO 1996/33978 (Zeneca); WO 1996/3397 (Zeneca) and WO 1996/33980 (Zeneca).

The term “anthracycline” relates to a chemotherapeutic agent, an anticancer agent for inducing apoptosis, preferably by inhibiting the rebinding of DNA in topoisomerase II. Examples include doxorubicin (adriamycin), daunorubicin (daunomycin), epirubicin, idarubicin, rhodomycin, pyrarubicin, valrubicin, N-trifluoro-acetyl doxorubicin-14-valerate, aclacinomycin, morpholinodoxorubicin (morpholino-DOX), cyanomorpholino-doxorubicin (cyanomorpholino-DOX), 2-pyrrolino-doxorubicin (2-PDOX), 5-iminodaunomycin, mitoxantrone and aclacinomycin A (aclarubicin). In some aspects, the anthracycline is administered in combination with an alkylating agent, e.g., doxorubicin in combination with cyclophosphamide (treatment with AC).

An “alkylating agent” as used herein is a chemotherapeutic agent which causes DNA damage by attaching an alkyl group to DNA. Alkylating agents include cyclophosphamide and N,N′,N″-triethylenethiophosphoramide.

An “effective amount” of a compound, for example, an anti-TIGIT antagonist antibody, a PD-1 axis binding antagonist (e.g., anti-PD-L1 antibody), a VEGF antagonist, a chemotherapeutic agent (e.g., a platinum-based chemotherapeutic agent or non-platinum-based chemotherapeutic agent), an ADC (e.g., enfortumab vedotin or sacituzumab govitecan), or a CSF (e.g., pegfilgrastim, filgrastim, or sargramostim), is at least the minimum amount required to achieve the desired therapeutic result, such as a measurable increase in overall survival or progression-free survival of a particular disease or disorder (e.g., a cancer). An effective amount herein may vary according to factors such as the disease state, age, sex, and weight of the patient, and the ability of the antibody to elicit a desired response in the subject. An effective amount is also one in which any toxic or detrimental effects of the treatment are outweighed by the therapeutically beneficial effects. For prophylactic use, beneficial or desired results include results such as eliminating or reducing the risk, lessening the severity, or delaying the onset of the disease, including biochemical, histological and/or behavioral symptoms of the disease, its complications, and intermediate pathological phenotypes presenting during development of the disease. For therapeutic use, beneficial or desired results include clinical results such as decreasing one or more symptoms resulting from the disease (e.g., reduction or delay in cancer-related pain, symptomatic skeletal-related events (SSE), reduction in symptoms per the European Organization for Research and Treatment of Cancer Quality-of-Life Questionnaire (EORTC QLQ-C30, e.g., fatigue, nausea, vomiting, pain, dyspnea, insomnia, appetite loss, constipation, diarrhea, or general level of physical emotional, cognitive, or social functioning), reduction in pain as measured by, e.g., the 10-point pain severity (measured at its worst) numerical rating scale (NRS), increasing the quality of life of those suffering from the disease, decreasing the dose of other medications required to treat the disease, enhancing effect of another medication such as via targeting, delaying the progression of the disease (e.g., progression-free survival or radiographic progression-free survival (rPFS); delay of unequivocal clinical progression (e.g., cancer-related pain progression, symptomatic skeletal-related event, deterioration in Eastern Cooperative Group Oncology Group (ECOG) Performance Status (PS) (e.g., how the disease affects the daily living abilities of the patient), and/or initiation of next systemic anti-cancer therapy), and/or prolonging survival. In the case of cancer or tumor, an effective amount of the drug may have the effect in reducing the number of cancer cells; reducing the tumor size; inhibiting (i.e., slow to some extent or desirably stop) cancer cell infiltration into peripheral organs; inhibit (i.e., slow to some extent and desirably stop) tumor metastasis; inhibiting to some extent tumor growth; and/or relieving to some extent one or more of the symptoms associated with the disorder. An effective amount can be administered in one or more administrations. For purposes of this invention, an effective amount of drug, compound, or pharmaceutical composition is an amount sufficient to accomplish prophylactic or therapeutic treatment either directly or indirectly. As is understood in the clinical context, an effective amount of a drug, compound, or pharmaceutical composition may or may not be achieved in conjunction with another drug, compound, or pharmaceutical composition. Thus, an “effective amount” may be considered in the context of administering one or more therapeutic agents, and a single agent may be considered to be given in an effective amount if, in conjunction with one or more other agents, a desirable result may be or is achieved. In some aspects, beneficial or desired results are reduction in symptoms associated with lung cancer per the health-related quality of life (HRQoL) questionnaire as assessed by symptoms in lung cancer (SILO) scale (e.g., time to deterioration (TTD) in cough dyspnea and chest pain) and/or delaying time to lung-specific antigen progression).

“Immunogenicity” refers to the ability of a particular substance to provoke an immune response. Tumors are immunogenic and enhancing tumor immunogenicity aids in the clearance of the tumor cells by the immune response. Examples of enhancing tumor immunogenicity include but are not limited to treatment with a TIGIT and/or PD-L1 antagonist (e.g., anti-TIGIT antagonist antibodies and/or anti-PD-L1 antibodies).

“Individual response” or “response” can be assessed using any endpoint indicating a benefit to the subject, including, without limitation, (1) inhibition, to some extent, of disease progression (e.g., progression of cancer, e.g., a lung cancer (e.g., small cell lung cancer (SCLC), which includes extensive stage SCLC (ES-SCLC); non-small cell lung cancer (NSCLC), which includes squamous NSCLC or non-squamous NSCLC, including locally advanced unresectable NSCLC (e.g., Stage IIIB NSCLC), or recurrent or metastatic NSCLC (e.g., Stage IV NSCLC), adenocarcinoma of the lung), a UC, e.g., a bladder cancer (e.g., an MIBC), a urothelial bladder cancer (UBC), a pancreatic cancer (e.g., a pancreatic ductal adenocarcinoma (PDAC), e.g., a metastatic PDAC)), a kidney or renal cancer (e.g., a renal cell carcinoma (RCC)), a melanoma, a head and neck cancer (e.g., a head and neck squamous cell cancer (HNSCC)), an ovarian cancer (0C), a gastric cancer (GC) (e.g., a gastroesophageal junction (GEJ) cancer), a hepatocellular carcinoma (HCC), a colorectal cancer (CRC; e.g., CRC with microsatellite-stable (MSS) and microsatellite instability (MSI) low (MSI-Low)), or a breast cancer (e.g., HER2+breast cancer and triple-negative breast cancer (TNBC), which are estrogen receptors (ER−), progesterone receptors (PgR−), and HER2 (HER2−) negative))), including slowing down and complete arrest; (2) a reduction in tumor size; (3) inhibition (i.e., reduction, slowing down or complete stopping) of cancer cell infiltration into adjacent peripheral organs and/or tissues; (4) inhibition (i.e. reduction, slowing down or complete stopping) of metastasis; (5) relief, to some extent, of one or more symptoms associated with the disease or disorder (e.g., cancer); (6) increase or extend in the length of survival, including overall survival and progression-free survival; and/or (9) decreased mortality at a given point of time following treatment.

As used herein, “pathological complete response” (pCR) is defined as the proportion of patients with an absence of residual invasive cancer of the complete resected specimen. In the context of breast cancer, “pathological complete response” or “pCR” refers to eradication of tumor from both breast and lymph nodes (ypT0/is ypN0).

As used herein in the context of urothelial carcinoma (UC), “pathological downstaging rate” is defined as the proportion of patients that reach ≥pT1pN0 at the time of cystectomy.

As used herein, “complete response” or “CR” refers to disappearance of all target lesions.

As used herein, “partial response” or “PR” refers to at least a 30% decrease in the sum of the longest diameters (SLD) of target lesions, taking as reference the baseline SLD.

As used herein, “objective response rate” (ORR) refers to the sum of complete response (CR) rate and partial response (PR) rate.

As used herein, “duration of objective response” (DOR) is defined as the time from the first occurrence of a documented objective response to disease progression, or death from any cause within 30 days of the last dose of a treatment, whichever occurs first.

“Sustained response” refers to the sustained effect on reducing tumor growth after cessation of a treatment. For example, the tumor size may remain to be the same or smaller as compared to the size at the beginning of the administration phase. In some aspects, the sustained response has a duration at least the same as the treatment duration, at least 1.5×, 2.0×, 2.5×, or 3.0× length of the treatment duration.

An “effective response” of a subject or a subject's “responsiveness” to treatment with a medicament and similar wording refers to the clinical or therapeutic benefit imparted to a subject as risk for, or suffering from, a disease or disorder, such as cancer. In one aspect, such benefit includes any one or more of: extending survival (including overall survival and progression free survival); resulting in an objective response (including a CR or a PR); or improving signs or symptoms of cancer.

A subject who “does not have an effective response” to treatment refers to a subject who does not have any one of extending survival (including overall survival and progression free survival); resulting in an objective response (including a CR or a PR); or improving signs or symptoms of cancer.

As used herein, “survival” refers to the patient remaining alive, and includes overall survival as well as progression-free survival.

As used herein, “overall survival” and “OS” refer to the length of time from either the date of diagnosis or the start of treatment for a disease (e.g., cancer) that the patient is still alive. For example, OS may be defined as the time from randomization to death from any cause.

As used herein, “overall survival rate” refers to the percentage of subjects in a group who are alive after a particular duration of time, e.g., six months, 1 year, or 5 years from the time of diagnosis or treatment.

As used herein, “recurrence-free survival” (RFS) is defined as the time from Day 1 in the first cycle after surgery to the first documented recurrence of disease or death from any cause.

As used herein, “event-free survival” (EFS) is defined as the time from randomization to any of the following events (whichever occurs first): disease progression (e.g., progression that precludes surgery, as assessed by the investigator); local or distant disease recurrence; or death from any cause.

As used herein, “progression-free survival” (PFS) refers to the length of time during and after treatment during which the disease being treated (e.g., cancer) does not get worse. Progression-free survival may include the amount of time patients have experienced a CR or a PR, as well as the amount of time patients have experienced stable disease.

As used herein, “stable disease” or “SD” refers to neither sufficient shrinkage of target lesions to qualify for CR or PR, nor sufficient increase to qualify for PD, taking as reference the smallest SLD since the treatment started.

As used herein, “progressive disease” or “PD” refers to at least a 20% increase in the sum of the longest diameters (SLD) of target lesions, taking as reference the smallest SLD recorded since the treatment started or the presence of one or more new lesions.

As used herein, “major pathological response” (MPR) is defined as 10% residual viable tumor at the time of surgical resection in the primary tumor.

As used herein, “delaying progression” of a disorder or disease means to defer, hinder, slow, retard, stabilize, and/or postpone development of the disease or disorder (e.g., cancer). This delay can be of varying lengths of time, depending on the history of the disease and/or subject being treated. As is evident to one skilled in the art, a sufficient or significant delay can, in effect, encompass prevention, in that the subject does not develop the disease. For example, in a late stage cancer, development of central nervous system (CNS) metastasis, may be delayed.

As used herein, the term “reducing or inhibiting cancer relapse” means to reduce or inhibit tumor or cancer relapse, or tumor or cancer progression.

By “reduce or inhibit” is meant the ability to cause an overall decrease of 20%, 30%, 40%, 50%, 60%, 70%, 75%, 80%, 85%, 90%, 95%, or greater. Reduce or inhibit can refer to the symptoms of the disorder being treated (e.g., cancer), the presence or size of metastases, or the size of the primary tumor.

By “extending survival” is meant increasing overall or progression free survival in a treated patient relative to an untreated patient (e.g., relative to a patient not treated with the medicament), or relative to a patient who does not express a biomarker at the designated level, and/or relative to a patient treated with an approved anti-tumor agent. An objective response refers to a measurable response, including CR or PR.

The terms “detecting” and “detection” are used herein in the broadest sense to include both qualitative and quantitative measurements of a target molecule. Detecting includes identifying the mere presence of the target molecule in a sample as well as determining whether the target molecule is present in the sample at detectable levels. Detecting may be direct or indirect.

As used herein, a “PD-L1-positive tumor cell fraction” is the percentage of viable tumor cells showing partial or complete membrane staining (exclusive of cytoplasmic staining) at any intensity relative to all viable tumor cells present in a sample, following staining of the sample in the context of an immunohistochemical (INC) assay, e.g., an IHC assay staining for PD-L1 using the antibody SP142, SP263, 22C3, or 28-8. Accordingly, a PD-L1-positive tumor cell fraction may be calculated using the PD-L1 IHC SP263 (Ventana) assay, for example, by the formula PD-L1-positive tumor cell fraction=(number of PD-L1-positive tumor cells)/(total number of PD-L1-positive and PD-L1 negative tumor cells), wherein PD-L1 cytoplasmic staining of tumor cells and all non-tumor cells (e.g., tumor-infiltrating immune cells, normal cells, necrotic cells, and debris) are excluded from evaluation and scoring. It will be appreciated that any given diagnostic PD-L1 antibody may correspond with a particular IHC assay protocol and/or scoring terminology that can be used to derive a PD-L1-positive tumor cell fraction. For example, a PD-L1-positive tumor cell fraction can be derived from a tumor cell sample stained with SP263, 22C3, SP142, or 28-8 using OPTIVIEW® detection on Benchmark ULTRA, EnVision Flex on AutostainerLink 48, OPTIVIEW® detection and amplification on Benchmark ULTRA, or EnVision Flex on AutostainerLink 48, respectively. In another example, a PD-L1-positive tumor cell fraction may be calculated using the PD-L1 IHC 22C3 pharmDx assay (Dako) according to the formula above. A skilled artisan will appreciate that the sensitivities can vary between different PD-L1 antibodies used in IHC assays. For example, only about 64% of samples that meet a 1% TC or 25% TC threshold, as defined respectively by staining with 28-8 or 22C3 and SP263, meet the threshold when stained using SP142. Hirsch et al., Journal of Thoracic Oncology 2016, 12(2): 208-222. As used herein, the terms PD-L1-positive tumor cell fraction and “tumor proportion score” (TPS) are used interchangeably.

As used herein, the “Ventana SP142 IHC assay” is conducted according to the Ventana PD-L1 (SP142) Assay package insert (Tucson, Ariz.: Ventana Medical Systems, Inc.), which is incorporated herein by reference in its entirety.

As used herein, the “Ventana SP263 IHC assay” is conducted according to the Ventana PD-L1 (SP263) Assay package insert (Tucson, Ariz.: Ventana Medical Systems, Inc.), which is incorporated herein by reference in its entirety.

As used herein, the “pharmDx 22C3 IHC assay” is conducted according to the PD-L1 IHC 22C3 pharmDx package insert (Carpinteria, Calif.: Dako, Agilent Pathology Solutions), which is incorporated herein by reference in its entirety.

As used herein, the “pharmDx 28-8 IHC assay” is conducted according to the PD-L1 IHC 28-8 pharmDx package insert (Carpinteria, Calif.: Dako, Agilent Pathology Solutions), which is incorporated herein by reference in its entirety.

A “tumor-infiltrating immune cell,” as used herein, refers to any immune cell present in a tumor or a sample thereof. Tumor-infiltrating immune cells include, but are not limited to, intratumoral immune cells, peritumoral immune cells, other tumor stroma cells (e.g., fibroblasts), or any combination thereof. Such tumor-infiltrating immune cells can be, for example, T lymphocytes (such as CD8+T lymphocytes and/or CD4+T lymphocytes), B lymphocytes, or other bone marrow-lineage cells, including granulocytes (e.g., neutrophils, eosinophils, and basophils), monocytes, macrophages, dendritic cells (e.g., interdigitating dendritic cells), histiocytes, and natural killer cells.

The term “biomarker,” as used herein, refers to an indicator, e.g., predictive, diagnostic, and/or prognostic, which can be detected in a sample, for example, PD-L1, ctDNA, or cytokines (e.g., cytokines associated with T-cell activation and/or lymphocyte subpopulations). The biomarker may serve as an indicator of a particular subtype of a disease or disorder (e.g., cancer) characterized by certain, molecular, pathological, histological, and/or clinical features. In some aspects, a biomarker is a gene. Biomarkers include, but are not limited to, polypeptides, polynucleotides (e.g., DNA (e.g., ctDNA), and/or RNA), polynucleotide copy number alterations (e.g., DNA copy numbers), polypeptide and polynucleotide modifications (e.g., posttranslational modifications), carbohydrates, and/or glycolipid-based molecular markers. In some aspects, the biomarker is PD-L1. In some aspects, the biomarker is ctDNA. In some aspects, the biomarker is one or more cytokines (e.g., one or more cytokines associated with T-cell activation and/or lymphocyte subpopulations). In some aspects, the biomarker is a cell (e.g., an immune cell, e.g., a T cell, e.g., a T cell subset, e.g., an activated T cell). In some aspects, the biomarker is a direct or indirect indicator of human papillomavirus (HPV) status. In some aspects, the biomarker is p16. In some aspects, the biomarker is an HPV protein or nucleic acid.

The term “housekeeping biomarker” refers to a biomarker or group of biomarkers (e.g., polynucleotides and/or polypeptides) which are typically similarly present in all cell types. In some aspects, the housekeeping biomarker is a “housekeeping gene.” A “housekeeping gene” refers herein to a gene or group of genes which encode proteins whose activities are essential for the maintenance of cell function and which are typically similarly present in all cell types.

As used herein, “circulating tumor DNA” and “ctDNA” refer to tumor-derived DNA in the circulatory system that is not associated with cells. ctDNA is a type of cell-free DNA (cfDNA) that may originate from tumor cells or from circulating tumor cells (CTCs). ctDNA may be found, e.g., in the bloodstream of a patient, or in a biological sample (e.g., blood, serum, plasma, or urine) obtained from a patient. In some aspects, ctDNA may include aberrant mutations (e.g., patient-specific variants) and/or methylation patterns.

The term “antibody” includes monoclonal antibodies (including full-length antibodies which have an immunoglobulin Fc region), polyclonal antibodies, antibody compositions with polyepitopic specificity, multispecific antibodies (e.g., bispecific antibodies), diabodies, and single-chain molecules, as well as antibody fragments, including antigen-binding fragments, such as Fab, F(ab′)2, and Fv, so long as they exhibit the desired biological activity. The term “immunoglobulin” (Ig) is used interchangeably with “antibody” herein. In one instance, the antibody is a full-length monoclonal antibody.

The term IgG “isotype” or “subclass” as used herein is meant any of the subclasses of immunoglobulins defined by the chemical and antigenic characteristics of their constant regions.

Depending on the amino acid sequences of the constant domains of their heavy chains, antibodies (immunoglobulins) can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1, and IgA2. The heavy chain constant domains that correspond to the different classes of immunoglobulins are called α, γ, ε, γ, and μ, respectively. The subunit structures and three-dimensional configurations of different classes of immunoglobulins are well known and described generally in, for example, Abbas et al. Cellular and Mol. Immunology, 4th ed. (W.B. Saunders, Co., 2000). An antibody may be part of a larger fusion molecule, formed by covalent or non-covalent association of the antibody with one or more other proteins or peptides.

The basic 4-chain antibody unit is a heterotetrameric glycoprotein composed of two identical light (L) chains and two identical heavy (H) chains. An IgM antibody consists of 5 of the basic heterotetramer units along with an additional polypeptide called a J chain, and contains 10 antigen binding sites, while IgA antibodies comprise from 2-5 of the basic 4-chain units which can polymerize to form polyvalent assemblages in combination with the J chain. In the case of IgGs, the 4-chain unit is generally about 150,000 Daltons. Each L chain is linked to an H chain by one covalent disulfide bond, while the two H chains are linked to each other by one or more disulfide bonds depending on the H chain isotype. Each H and L chain also has regularly spaced intrachain disulfide bridges. Each H chain has at the N-terminus, a variable domain (V_{H}) followed by three constant domains (C_{H}) for each of the α and γ chains and four C_{H} domains for μ and ε isotypes. Each L chain has at the N-terminus, a variable domain (V_{L}) followed by a constant domain at its other end. The V_{L} is aligned with the V_{H} and the C_{L} is aligned with the first constant domain of the heavy chain (CH1). Particular amino acid residues are believed to form an interface between the light chain and heavy chain variable domains. The pairing of a V_{H} and V_{L} together forms a single antigen-binding site. For the structure and properties of the different classes of antibodies, see, e.g., Basic and Clinical Immunology, 8^{th} Edition, Daniel P. Sties, Abba I. Terr and Tristram G. Parsolw (eds), Appleton & Lange, Norwalk, Conn., 1994, page 71 and Chapter 6. The L chain from any vertebrate species can be assigned to one of two clearly distinct types, called kappa and lambda, based on the amino acid sequences of their constant domains. Depending on the amino acid sequence of the constant domain of their heavy chains (CH), immunoglobulins can be assigned to different classes or isotypes. There are five classes of immunoglobulins: IgA, IgD, IgE, IgG and IgM, having heavy chains designated α, δ, ε, γ, and μ, respectively. The γ and α classes are further divided into subclasses on the basis of relatively minor differences in the CH sequence and function, e.g., humans express the following subclasses: IgG1, IgG2A, IgG2B, IgG3, IgG4, IgA1 and IgA2.

The term “hypervariable region” or “HVR” as used herein refers to each of the regions of an antibody variable domain which are hypervariable in sequence and which determine antigen binding specificity, for example “complementarity determining regions” (“CDRs”).

Generally, antibodies comprise six CDRs: three in the VH (CDR-H1, CDR-H2, CDR-H3), and three in the VL (CDR-L1, CDR-L2, CDR-L3). Exemplary CDRs herein include:

(a) hypervariable loops occurring at amino acid residues 26-32 (L1), 50-52 (L2), 91-96 (L3), 26-32 (H1), 53-55 (H2), and 96-101 (H3) (Chothia and Lesk, J. Mol. Biol. 196:901-917 (1987));

(b) CDRs occurring at amino acid residues 24-34 (L1), 50-56 (L2), 89-97 (L3), 31-35b (H1), 50-65 (H2), and 95-102 (H3) (Kabat et al., Sequences of Proteins of Immunological Interest, 5^{th} Ed. Public Health Service, National Institutes of Health, Bethesda, Md. (1991)); and

(c) antigen contacts occurring at amino acid residues 27c-36 (L1), 46-55 (L2), 89-96 (L3), 30-35b (H1), 47-58 (H2), and 93-101 (H3) (MacCallum et al. J. Mol. Biol. 262: 732-745 (1996)).

Unless otherwise indicated, the CDRs are determined according to Kabat et al., supra. One of skill in the art will understand that the CDR designations can also be determined according to Chothia, supra, McCallum, supra, or any other scientifically accepted nomenclature system.

“Framework” or “FR” refers to variable domain residues other than complementary determining regions (CDRs). The FR of a variable domain generally consists of four FR domains: FR1, FR2, FR3, and FR4. Accordingly, the CDR and FR sequences generally appear in the following sequence in VH (or VL): FR1-CDR-H1(CDR-L1)-FR2-CDR-H2(CDR-L2)-FR3-CDR-H3(CDR-L3)-FR4.

The term “variable-domain residue-numbering as in Kabat” or “amino-acid-position numbering as in Kabat,” and variations thereof, refers to the numbering system used for heavy-chain variable domains or light-chain variable domains of the compilation of antibodies in Kabat et al., supra. Using this numbering system, the actual linear amino acid sequence may contain fewer or additional amino acids corresponding to a shortening of, or insertion into, a FR or HVR of the variable domain. For example, a heavy-chain variable domain may include a single amino acid insert (e.g., residue 52a according to Kabat) after residue 52 of H2 and inserted residues (e.g., residues 82a, 82b, and 82c, etc. according to Kabat) after heavy-chain FR residue 82. The Kabat numbering of residues may be determined for a given antibody by alignment at regions of homology of the sequence of the antibody with a “standard” Kabat numbered sequence.

The Kabat numbering system is generally used when referring to a residue in the variable domain (approximately residues 1-107 of the light chain and residues 1-113 of the heavy chain) (e.g., Kabat et al., Sequences of Immunological Interest. 5th Ed. Public Health Service, National Institutes of Health, Bethesda, Md. (1991)). The “EU numbering system” or “EU index” is generally used when referring to a residue in an immunoglobulin heavy chain constant region (e.g., the EU index reported in Kabat et al., supra). The “EU index as in Kabat” refers to the residue numbering of the human IgG1 EU antibody.

The term “variable” refers to the fact that certain segments of the variable domains differ extensively in sequence among antibodies. The V domain mediates antigen binding and defines the specificity of a particular antibody for its particular antigen. However, the variability is not evenly distributed across the entire span of the variable domains. Instead, it is concentrated in three segments called hypervariable regions (HVRs) both in the light-chain and the heavy chain variable domains. The more highly conserved portions of variable domains are called the framework regions (FR). The variable domains of native heavy and light chains each comprise four FR regions, largely adopting a beta-sheet configuration, connected by three HVRs, which form loops connecting, and in some cases forming part of, the beta-sheet structure. The HVRs in each chain are held together in close proximity by the FR regions and, with the HVRs from the other chain, contribute to the formation of the antigen binding site of antibodies (see Kabat et al., Sequences of Immunological Interest, Fifth Edition, National Institute of Health, Bethesda, Md. (1991)). The constant domains are not involved directly in the binding of antibody to an antigen, but exhibit various effector functions, such as participation of the antibody in antibody-dependent cellular toxicity.

The “variable region” or “variable domain” of an antibody refers to the amino-terminal domains of the heavy or light chain of the antibody. The variable domains of the heavy chain and light chain may be referred to as “VH” and “VL”, respectively. These domains are generally the most variable parts of the antibody (relative to other antibodies of the same class) and contain the antigen binding sites.

“Framework” or “FR” refers to variable domain residues other than hypervariable region (HVR) residues. The FR of a variable domain generally consists of four FR domains: FR1, FR2, FR3, and FR4. Accordingly, the HVR and FR sequences generally appear in the following sequence in VH (or VL): FR1-H1(L1)-FR2-H2(L2)-FR3-H3(L3)-FR4.

The terms “full-length antibody,” “intact antibody,” and “whole antibody” are used interchangeably to refer to an antibody in its substantially intact form, as opposed to an antibody fragment. Specifically, whole antibodies include those with heavy and light chains including an Fc region. The constant domains may be native sequence constant domains (e.g., human native sequence constant domains) or amino acid sequence variants thereof. In some cases, the intact antibody may have one or more effector functions.

A “naked antibody” refers to an antibody that is not conjugated to a heterologous moiety (e.g., a cytotoxic moiety) or radiolabel. The naked antibody may be present in a pharmaceutical composition.

An “antibody fragment” comprises a portion of an intact antibody, preferably the antigen-binding and/or the variable region of the intact antibody. Examples of antibody fragments include Fab, Fab′, F(ab′)2 and Fv fragments; diabodies; linear antibodies (see U.S. Pat. No. 5,641,870, Example 2; Zapata et al., Protein Eng. 8(10): 1057-1062 [1995]); single-chain antibody molecules and multispecific antibodies formed from antibody fragments. Papain digestion of antibodies produced two identical antigen-binding fragments, called “Fab” fragments, and a residual “Fc” fragment, a designation reflecting the ability to crystallize readily. The Fab fragment consists of an entire L chain along with the variable region domain of the H chain (V_{H}), and the first constant domain of one heavy chain (C_{H}1). Each Fab fragment is monovalent with respect to antigen binding, i.e., it has a single antigen-binding site. Pepsin treatment of an antibody yields a single large F(ab′)₂ fragment which roughly corresponds to two disulfide linked Fab fragments having different antigen-binding activity and is still capable of cross-linking antigen. Fab′ fragments differ from Fab fragments by having a few additional residues at the carboxy terminus of the CH1 domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments which have hinge cysteines between them. Other chemical couplings of antibody fragments are also known.

The Fc fragment comprises the carboxy-terminal portions of both H chains held together by disulfides. The effector functions of antibodies are determined by sequences in the Fc region, the region which is also recognized by Fc receptors (FcR) found on certain types of cells.

“Functional fragments” of the antibodies of the invention comprise a portion of an intact antibody, generally including the antigen binding or variable region of the intact antibody or the Fc region of an antibody which retains or has modified FcR binding capability. Examples of antibody fragments include linear antibody, single-chain antibody molecules and multispecific antibodies formed from antibody fragments.

“Fv” is the minimum antibody fragment which contains a complete antigen-recognition and -binding site. This fragment consists of a dimer of one heavy- and one light-chain variable region domain in tight, non-covalent association. From the folding of these two domains emanate six hypervariable loops (3 loops each from the H and L chain) that contribute the amino acid residues for antigen binding and confer antigen binding specificity to the antibody. However, even a single variable domain (or half of an Fv comprising only three HVRs specific for an antigen) has the ability to recognize and bind antigen, although at a lower affinity than the entire binding site.

“Single-chain Fv” also abbreviated as “sFv” or “scFv” are antibody fragments that comprise the V_{H} and V_{L} antibody domains connected into a single polypeptide chain. Preferably, the sFv polypeptide further comprises a polypeptide linker between the V_{H} and V_{L} domains which enables the sFv to form the desired structure for antigen binding. For a review of the sFv, see Pluckthun in The Pharmacology of Monoclonal Antibodies, vol. 113, Rosenburg and Moore eds., Springer-Verlag, New York, pp. 269-315 (1994).

The term “Fc region” herein is used to define a C-terminal region of an immunoglobulin heavy chain that contains at least a portion of the constant region. The term includes native sequence Fc regions and variant Fc regions. In one aspect, a human IgG heavy chain Fc region extends from Cys226, or from Pro230, to the carboxyl-terminus of the heavy chain. However, antibodies produced by host cells may undergo post-translational cleavage of one or more, particularly one or two, amino acids from the C-terminus of the heavy chain. Therefore, an antibody produced by a host cell by expression of a specific nucleic acid molecule encoding a full-length heavy chain may include the full-length heavy chain, or it may include a cleaved variant of the full-length heavy chain. This may be the case where the final two C-terminal amino acids of the heavy chain are glycine (G446) and lysine (K447). Therefore, the C-terminal lysine (Lys447), or the C-terminal glycine (Gly446) and lysine (Lys447), of the Fc region may or may not be present. Amino acid sequences of heavy chains including an Fc region are denoted herein without the C-terminal lysine (Lys447) if not indicated otherwise. In one aspect, a heavy chain including an Fc region as specified herein, comprised in an antibody disclosed herein, comprises an additional C-terminal glycine-lysine dipeptide (G446 and K447). In one aspect, a heavy chain including an Fc region as specified herein, comprised in an antibody disclosed herein, comprises an additional C-terminal glycine residue (G446). In one aspect, a heavy chain including an Fc region as specified herein, comprised in an antibody disclosed herein, comprises an additional C-terminal lysine residue (K447). In one aspect, the Fc region contains a single amino acid substitution N297A of the heavy chain. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, Md., 1991.

“Fc receptor” or “FcR” describes a receptor that binds to the Fc region of an antibody. The preferred FcR is a native sequence human FcR. Moreover, a preferred FcR is one which binds an IgG antibody (a gamma receptor) and includes receptors of the FcγRI, FcγRII, and FcγRIII subclasses, including allelic variants and alternatively spliced forms of these receptors, FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (ITAM) in its cytoplasmic domain. Inhibiting receptor FcγRIIB contains an immunoreceptor tyrosine-based inhibition motif (ITIM) in its cytoplasmic domain (see M. Daëron, Annu. Rev. Immunol. 15:203-234 (1997). FcRs are reviewed in Ravetch and Kinet, Annu. Rev. Immunol. 9: 457-92 (1991); Capel et al., Immunomethods 4: 25-34 (1994); and de Haas et al., J. Lab. Clin. Med. 126: 330-41 (1995). Other FcRs, including those to be identified in the future, are encompassed by the term “FcR” herein.

The term “diabodies” refers to small antibody fragments prepared by constructing sFv fragments (see preceding paragraph) with short linkers (about 5-10) residues) between the V_{H} and V_{L} domains such that inter-chain but not intra-chain pairing of the V domains is achieved, thereby resulting in a bivalent fragment, i.e., a fragment having two antigen-binding sites. Bispecific diabodies are heterodimers of two “crossover” sFv fragments in which the V_{H} and V_{L} domains of the two antibodies are present on different polypeptide chains. Diabodies are described in greater detail in, for example, EP 404,097; WO 93/11161; Hollinger et al., Proc. Natl. Acad. Sci. USA 90: 6444-6448 (1993).

The monoclonal antibodies herein specifically include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is (are) identical with or homologous to corresponding sequences in antibodies derived from another species or belonging to another antibody class or subclass, as well as fragments of such antibodies, so long as they exhibit the desired biological activity (U.S. Pat. No. 4,816,567; Morrison et al., Proc. Natl. Acad. Sci. USA, 81:6851-6855 (1984)). Chimeric antibodies of interest herein include PRIMATIZED® antibodies wherein the antigen-binding region of the antibody is derived from an antibody produced by, e.g., immunizing macaque monkeys with an antigen of interest. As used herein, “humanized antibody” is used a subset of “chimeric antibodies.”

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG₁, IgG₂, IgG₃, IgG₄, IgA₁, and IgA₂. The heavy chain constant domains that correspond to the different classes of immunoglobulins are called α, δ, ε, γ, and μ, respectively.

“Affinity” refers to the strength of the sum total of non-covalent interactions between a single binding site of a molecule (e.g., an antibody) and its binding partner (e.g., an antigen, e.g., TIGIT, PD-L1, or VEGF). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (K_{D}). Affinity can be measured by common methods known in the art, including those described herein. Specific illustrative and exemplary aspects for measuring binding affinity are described in the following.

A “human antibody” is an antibody that possesses an amino-acid sequence corresponding to that of an antibody produced by a human and/or has been made using any of the techniques for making human antibodies as disclosed herein. This definition of a human antibody specifically excludes a humanized antibody comprising non-human antigen-binding residues. Human antibodies can be produced using various techniques known in the art, including phage-display libraries. Hoogenboom and Winter, J. Mol. Biol., 227:381 (1991); Marks et al., J. Mol. Biol., 222:581 (1991). Also available for the preparation of human monoclonal antibodies are methods described in Cole et al., Monoclonal Antibodies and Cancer Therapy, Alan R. Liss, p. 77 (1985); Boerner et al., J. Immunol., 147(1):86-95 (1991). See also van Dijk and van de Winkel, Curr. Opin. Pharmacol., 5: 368-74 (2001). Human antibodies can be prepared by administering the antigen to a transgenic animal that has been modified to produce such antibodies in response to antigenic challenge, but whose endogenous loci have been disabled, e.g., immunized xenomice (see, e.g., U.S. Pat. Nos. 6,075,181 and 6,150,584 regarding XENOMOUSE™ technology). See also, for example, Li et al., Proc. Natl. Acad. Sci. USA, 103:3557-3562 (2006) regarding human antibodies generated via a human B-cell hybridoma technology.

“Humanized” forms of non-human (e.g., murine) antibodies are chimeric antibodies that contain minimal sequence derived from non-human immunoglobulin. In one aspect, a humanized antibody is a human immunoglobulin (recipient antibody) in which residues from an HVR (hereinafter defined) of the recipient are replaced by residues from an HVR of a non-human species (donor antibody) such as mouse, rat, rabbit or non-human primate having the desired specificity, affinity, and/or capacity. In some instances, framework (“FR”) residues of the human immunoglobulin are replaced by corresponding non-human residues. Furthermore, humanized antibodies may comprise residues that are not found in the recipient antibody or in the donor antibody. These modifications may be made to further refine antibody performance, such as binding affinity. In general, a humanized antibody will comprise substantially all of at least one, and typically two, variable domains, in which all or substantially all of the hypervariable loops correspond to those of a non-human immunoglobulin sequence, and all or substantially all of the FR regions are those of a human immunoglobulin sequence, although the FR regions may include one or more individual FR residue substitutions that improve antibody performance, such as binding affinity, isomerization, immunogenicity, etc. The number of these amino acid substitutions in the FR are typically no more than 6 in the H chain, and in the L chain, no more than 3. The humanized antibody optionally will also comprise at least a portion of an immunoglobulin constant region (Fc), typically that of a human immunoglobulin. For further details, see, e.g., Jones et al., Nature 321:522-525 (1986); Riechmann et al., Nature 332:323-329 (1988); and Presta, Curr. Op. Struct. Biol. 2:593-596 (1992). See also, for example, Vaswani and Hamilton, Ann. Allergy, Asthma & Immunol. 1:105-115 (1998); Harris, Biochem. Soc. Transactions 23:1035-1038 (1995); Hurle and Gross, Curr. Op. Biotech. 5:428-433 (1994); and U.S. Pat. Nos. 6,982,321 and 7,087,409.

The term an “isolated antibody” when used to describe the various antibodies disclosed herein, means an antibody that has been identified and separated and/or recovered from a cell or cell culture from which it was expressed. Contaminant components of its natural environment are materials that would typically interfere with diagnostic or therapeutic uses for the polypeptide, and can include enzymes, hormones, and other proteinaceous or non-proteinaceous solutes. In some aspects, an antibody is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC). For a review of methods for assessment of antibody purity, see, e.g., Flatman et al., J. Chromatogr. B 848:79-87 (2007). In preferred aspects, the antibody will be purified (1) to a degree sufficient to obtain at least 15 residues of N-terminal or internal amino acid sequence by use of a spinning cup sequenator, or (2) to homogeneity by SDS-PAGE under non-reducing or reducing conditions using Coomassie blue or, preferably, silver stain. Isolated antibody includes antibodies in situ within recombinant cells, because at least one component of the polypeptide natural environment will not be present. Ordinarily, however, isolated polypeptide will be prepared by at least one purification step.

The term “monoclonal antibody” as used herein refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies in accordance with the present invention may be made by a variety of techniques, including but not limited to the hybridoma method, recombinant DNA methods, phage-display methods, and methods utilizing transgenic animals containing all or part of the human immunoglobulin loci.

As used herein, the term “binds,” “specifically binds to,” or is “specific for” refers to measurable and reproducible interactions such as binding between a target and an antibody, which is determinative of the presence of the target in the presence of a heterogeneous population of molecules including biological molecules. For example, an antibody that specifically binds to a target (which can be an epitope) is an antibody that binds this target with greater affinity, avidity, more readily, and/or with greater duration than it binds to other targets. In one aspect, the extent of binding of an antibody to an unrelated target is less than about 10% of the binding of the antibody to the target as measured, for example, by a radioimmunoassay (RIA). In certain aspects, an antibody that specifically binds to a target has a dissociation constant (K_{D}) of ≤1 μM, ≤100 nM, ≤10 nM, ≤1 nM, or ≤0.1 nM. In certain aspects, an antibody specifically binds to an epitope on a protein that is conserved among the protein from different species. In another aspect, specific binding can include, but does not require exclusive binding. The term as used herein can be exhibited, for example, by a molecule having a K_{D} for the target of 10⁻⁴M or lower, alternatively 10⁻⁸M or lower, alternatively 10⁻⁶ M or lower, alternatively 10⁻⁷ M or lower, alternatively 10⁻⁸ M or lower, alternatively 10⁻⁹ M or lower, alternatively 10⁻¹⁰ M or lower, alternatively 10⁻¹¹ M or lower, alternatively 10⁻¹² M or lower or a K_{D} in the range of 10⁻⁴ M to 10⁻⁶ M or 10⁻⁶ M to 10⁻¹° M or 10⁷ M to 10⁻⁹ M. As will be appreciated by the skilled artisan, affinity and K_{D} values are inversely related. A high affinity for an antigen is measured by a low K_{D} value. In one aspect, the term “specific binding” refers to binding where a molecule binds to a particular polypeptide or epitope on a particular polypeptide without substantially binding to any other polypeptide or polypeptide epitope.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, Calif., or may be compiled from the source code. The ALIGN-2 program should be compiled for use on a UNIX operating system, including digital UNIX V4.0D. All sequence comparison parameters are set by the ALIGN-2 program and do not vary.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:

100 times the fraction X/Y

where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

As used herein, “subject” or “individual” is meant a mammal, including, but not limited to, a human or non-human mammal, such as a bovine, equine, canine, ovine, or feline. In some aspects, the subject is a human. Patients are also subjects herein.

The term “patient” refers to a human patient. For example, the patient may be an adult.

The term “diagnosis” is used herein to refer to the identification or classification of a molecular or pathological state, disease or condition (e.g., cancer). For example, “diagnosis” may refer to identification of a particular type of cancer. “Diagnosis” may also refer to the classification of a particular subtype of cancer, for instance, by histopathological criteria, or by molecular features (e.g., a subtype characterized by expression of one or a combination of biomarkers (e.g., particular genes or proteins encoded by said genes)).

The term “sample,” as used herein, refers to a composition that is obtained or derived from a subject and/or individual of interest that contains a cellular and/or other molecular entity that is to be characterized and/or identified, for example, based on physical, biochemical, chemical, and/or physiological characteristics. For example, the phrase “tumor sample”, “disease sample” and variations thereof refers to any sample obtained from a subject of interest that would be expected or is known to contain the cellular and/or molecular entity that is to be characterized. Samples include, but are not limited to, tissue samples, primary or cultured cells or cell lines, cell supernatants, cell lysates, platelets, serum, plasma, vitreous fluid, lymph fluid, synovial fluid, follicular fluid, seminal fluid, amniotic fluid, milk, whole blood, blood-derived cells, urine, cerebro-spinal fluid, saliva, sputum, tears, perspiration, mucus, stool, tumor lysates, and tissue culture medium, tissue extracts such as homogenized tissue, tumor tissue, cellular extracts, and combinations thereof.

By “tissue sample” or “cell sample” is meant a collection of similar cells obtained from a tissue of a subject or individual. The source of the tissue or cell sample may be solid tissue as from a fresh, frozen and/or preserved organ, tissue sample, biopsy, and/or aspirate; blood or any blood constituents such as plasma; bodily fluids such as cerebral spinal fluid, amniotic fluid, peritoneal fluid, or interstitial fluid; cells from any time in gestation or development of the subject. The tissue sample may also be primary or cultured cells or cell lines. Optionally, the tissue or cell sample is obtained from a disease tissue/organ. For instance, a “tumor sample” is a tissue sample obtained from a tumor or other cancerous tissue. The tissue sample may contain a mixed population of cell types (e.g., tumor cells and non-tumor cells, cancerous cells and non-cancerous cells). The tissue sample may contain compounds which are not naturally intermixed with the tissue in nature such as preservatives, anticoagulants, buffers, fixatives, wax, nutrients, antibiotics, or the like. In some aspects, the sample is a tumor tissue sample. In some aspects, the tumor tissue sample is a UC tumor tissue sample (e.g., a bladder cancer tumor tissue sample (e.g., an MIBC tumor tissue sample)). In some aspects, the sample is a transurethral resection of bladder tumor (TURBT) sample. In some aspects, the sample is a cystectomy or nephroureterectomy sample. In other aspects, the tumor tissue sample is a lung cancer tumor tissue sample (e.g., an early stage lung cancer tissue sample (e.g., an NSCLC tumor tissue sample (e.g., a stage II, IIIA, or IIIB NSCLC tumor tissue sample), e.g., squamous or non-squamous NSCLC tumor tissue sample, e.g., a resectable NSCLC tumor tissue sample)). In some aspects, the sample is a locally advanced unresectable NSCLC tumor tissue sample (e.g., Stage IIIB NSCLC tumor tissue sample), or recurrent or metastatic NSCLC tumor tissue sample (e.g., Stage IV NSCLC tumor tissue sample)), a pancreatic cancer tumor tissue sample (e.g., a PDAC tumor tissue sample), e.g., a metastatic PDAC tumor tissue sample)), or a breast cancer tumor tissue sample (e.g., a HER2+ breast cancer tumor tissue sample or a TNBC tumor tissue sample).

For the purposes herein a “section” of a tissue sample is meant a single part or piece of a tissue sample, for example, a thin slice of tissue or cells cut from a tissue sample (e.g., a tumor sample). It is to be understood that multiple sections of tissue samples may be taken and subjected to analysis, provided that it is understood that the same section of tissue sample may be analyzed at both morphological and molecular levels, or analyzed with respect to polypeptides (e.g., by immunohistochemistry) and/or polynucleotides (e.g., by in situ hybridization).

A “reference sample,” “reference cell,” “reference tissue,” “control sample,” “control cell,” or “control tissue,” as used herein, refers to a sample, cell, tissue, standard, or level that is used for comparison purposes. In one aspect, a reference sample, reference cell, reference tissue, control sample, control cell, or control tissue is obtained from a healthy and/or non-diseased part of the body (e.g., tissue or cells) of the same subject or individual. For example, the reference sample, reference cell, reference tissue, control sample, control cell, or control tissue may be healthy and/or non-diseased cells or tissue adjacent to the diseased cells or tissue (e.g., cells or tissue adjacent to a tumor). In another aspect, a reference sample is obtained from an untreated tissue and/or cell of the body of the same subject or individual. In yet another aspect, a reference sample, reference cell, reference tissue, control sample, control cell, or control tissue is obtained from a healthy and/or non-diseased part of the body (e.g., tissues or cells) of an individual who is not the subject or individual. In even another aspect, a reference sample, reference cell, reference tissue, control sample, control cell, or control tissue is obtained from an untreated tissue and/or cell of the body of an individual who is not the subject or individual.

The term “protein,” as used herein, refers to any native protein from any vertebrate source, including mammals such as primates (e.g., humans) and rodents (e.g., mice and rats), unless otherwise indicated. The term encompasses “full-length,” unprocessed protein as well as any form of the protein that results from processing in the cell. The term also encompasses naturally occurring variants of the protein, e.g., splice variants or allelic variants.

“Polynucleotide” or “nucleic acid,” as used interchangeably herein, refers to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase, or by a synthetic reaction. Thus, for instance, polynucleotides as defined herein include, without limitation, single- and double-stranded DNA, DNA including single- and double-stranded regions, single- and double-stranded RNA, and RNA including single- and double-stranded regions, hybrid molecules comprising DNA and RNA that may be single-stranded or, more typically, double-stranded or include single- and double-stranded regions. In addition, the term “polynucleotide” as used herein refers to triple-stranded regions comprising RNA or DNA or both RNA and DNA. The strands in such regions may be from the same molecule or from different molecules. The regions may include all of one or more of the molecules, but more typically involve only a region of some of the molecules. One of the molecules of a triple-helical region often is an oligonucleotide. The terms “polynucleotide” and “nucleic acid” specifically includes mRNA and cDNAs.

A polynucleotide may comprise modified nucleotides, such as methylated nucleotides and their analogs. If present, modification to the nucleotide structure may be imparted before or after assembly of the polymer. The sequence of nucleotides may be interrupted by non-nucleotide components. A polynucleotide may be further modified after synthesis, such as by conjugation with a label. Other types of modifications include, for example, “caps,” substitution of one or more of the naturally-occurring nucleotides with an analog, internucleotide modifications such as, for example, those with uncharged linkages (e.g., methyl phosphonates, phosphotriesters, phosphoamidates, carbamates, and the like) and with charged linkages (e.g., phosphorothioates, phosphorodithioates, and the like), those containing pendant moieties, such as, for example, proteins (e.g., nucleases, toxins, antibodies, signal peptides, poly-L-lysine, and the like), those with intercalators (e.g., acridine, psoralen, and the like), those containing chelators (e.g., metals, radioactive metals, boron, oxidative metals, and the like), those containing alkylators, those with modified linkages (e.g., alpha anomeric nucleic acids), as well as unmodified forms of the polynucleotide(s). Further, any of the hydroxyl groups ordinarily present in the sugars may be replaced, for example, by phosphonate groups, phosphate groups, protected by standard protecting groups, or activated to prepare additional linkages to additional nucleotides, or may be conjugated to solid or semi-solid supports. The 5′ and 3′ terminal OH can be phosphorylated or substituted with amines or organic capping group moieties of from 1 to 20 carbon atoms. Other hydroxyls may also be derivatized to standard protecting groups. Polynucleotides can also contain analogous forms of ribose or deoxyribose sugars that are generally known in the art, including, for example, 2′-O-methyl-, 2′-O-allyl-, 2′-fluoro-, or 2′-azido-ribose, carbocyclic sugar analogs, α-anomeric sugars, epimeric sugars such as arabinose, xyloses or lyxoses, pyranose sugars, furanose sugars, sedoheptuloses, acyclic analogs, and abasic nucleoside analogs such as methyl riboside. One or more phosphodiester linkages may be replaced by alternative linking groups. These alternative linking groups include, but are not limited to, aspects wherein phosphate is replaced by P(O)S (“thioate”), P(S)S (“dithioate”), “(O)NR₂ (“amidate”), P(O)R, P(O)OR′, CO or CH2 (“formacetal”), in which each R or R′ is independently H or substituted or unsubstituted alkyl (1-20 C) optionally containing an ether (—O—) linkage, aryl, alkenyl, cycloalkyl, cycloalkenyl or araldyl. Not all linkages in a polynucleotide need be identical. The preceding description applies to all polynucleotides referred to herein, including RNA and DNA.

“Carriers” as used herein include pharmaceutically acceptable carriers, excipients, or stabilizers that are nontoxic to the cell or mammal being exposed thereto at the dosages and concentrations employed. Often the physiologically acceptable carrier is an aqueous pH buffered solution. Examples of physiologically acceptable carriers include buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™, polyethylene glycol (PEG), and PLURONICS™.

The phrase “pharmaceutically acceptable” indicates that the substance or composition must be compatible chemically and/or toxicologically, with the other ingredients comprising a formulation, and/or the mammal being treated therewith.

The term “pharmaceutical formulation” or “pharmaceutical composition” refers to a preparation which is in such form as to permit the biological activity of an active ingredient contained therein to be effective, and which contains no additional components which are unacceptably toxic to a subject to which the formulation would be administered.

The term “package insert” is used to refer to instructions customarily included in commercial packages of therapeutic products or medicaments that contain information about the indications, usage, dosage, administration, combination therapy, other medicaments to be combined with the packaged product, and/or contraindications and/or warnings concerning the use of such therapeutic products or medicaments.

As used herein, the term “induction phase” refers to a series of one or more dosing cycles (e.g., about 4-6 cycles) of one or more therapeutic agents (e.g., an anti-TIGIT antagonist antibody, a PD-1 axis binding antagonist, and/or a chemotherapeutic agent) administered to a subject, wherein the one or more dosing cycles are optionally followed by a maintenance phase.

The term “maintenance phase” as used herein refers to a series of one or more dosing cycles of one or more therapeutic agents (e.g., an anti-TIGIT antagonist antibody, a PD-1 axis binding antagonist, and/or a chemotherapeutic agent) that are administered to a subject subsequent to an induction phase. In some instances, the maintenance phase is initiated only if the subject did not experience disease progression or unacceptable toxicity during the induction phase. The induction phase and maintenance phase may or may not comprise use of the same therapeutic agents. For example, in some instances, the induction phase includes use of an anti-TIGIT antagonist antibody, a PD-1 axis binding antagonist, a platinum-based chemotherapeutic agent, and a non-platinum-based chemotherapeutic agent, and the maintenance phase includes use of an anti-TIGIT antagonist antibody and a PD-1 axis binding antagonist.

In the context of bladder cancer, the term “ineligible for treatment with a platinum-based chemotherapy” or “unfit for treatment with a platinum-based chemotherapy” means that the subject is ineligible or unfit for treatment with a platinum-based chemotherapy, either in the attending clinician's judgment or according to standardized criteria for eligibility for platinum-based chemotherapy that are known in the art. For example, the criteria set forth in Galsky et al. Lancet Oncol. 12(3):211-4, 2011, which is incorporated herein by reference in its entirety, may be used to determine whether a subject is eligible for cisplatin-based chemotherapy. Galsky et al. describe a consensus definition of patients with metastatic UC (mUC) in which patients meeting at least one of the following are considered unfit for cisplatin-based chemotherapy: (i) a World Health Association (WHO) or Eastern Cooperative Oncology Group (ECOG) performance status of 2, or Karnofsky performance status of 60-70%; (ii) creatinine clearance (calculated or measured) less than 1 mL/s; (iii) National Cancer Institute (NCI) Common Terminology Criteria for Adverse Events (CTCAE) v4.0 Grade ≥2 audiometric hearing loss; (iv) CTCAE v.4.0 Grade ≥2 peripheral neuropathy; and/or New York Heart Association (NYHA) class III heart failure. In one example, a patient is considered unfit for cisplatin-based chemotherapy if they have one or more of the following: impaired renal function (e.g., glomerular filtration rate (GFR) >30 but <60 mL/min); GFR may be assessed by direct measurement (i.e., creatinine clearance or ethyldediaminetetra-acetate) or, if not available, by calculation from serum/plasma creatinine (Cockcroft-Gault formula)); hearing loss (e.g., NCI CTCAE v4.0 Grade ≥2 audiometric hearing loss of 25 decibels at two contiguous frequencies); peripheral neuropathy (e.g., NCI CTCAE v4.0 Grade ≥2 peripheral neuropathy (i.e., sensory alteration or paresthesia, including tingling)); and/or ECOG performance status assessment (see Oken et al. Am. J. Clin. Oncol. 5:649-655, 1982, which is incorporated herein by reference in its entirety) (e.g., an ECOG performance status of 2). In some aspects, a subject having one of the following may be eligible for carboplatin-based chemotherapy: impaired renal function (e.g., GFR >30 but <60 mL/min); GFR may be assessed by direct measurement (i.e., creatinine clearance or ethyldediaminetetra-acetate) or, if not available, by calculation from serum/plasma creatinine (Cockcroft-Gault formula)); hearing loss (e.g., CTCAE v4.0 Grade ≥2 audiometric hearing loss of 25 decibels at two contiguous frequencies); peripheral neuropathy (e.g., NCI CTCAE v4.0 Grade ≥2 peripheral neuropathy (i.e., sensory alteration or paresthesia, including tingling)); and/or ECOG performance status assessment (e.g., an ECOG performance status of 2). For example, cisplatin ineligibility may be defined by any one of the following criteria: (i) impaired renal function (GFR <60 mL/min); GFR may be assessed by direct measurement (i.e., creatinine clearance or ethyldediaminetetra-acetate) or, if not available, by calculation from serum/plasma creatinine (Cockcroft Gault formula); (ii) a hearing loss (measured by audiometry) of 25 dB at two contiguous frequencies; (iii) Grade 2 or greater peripheral neuropathy (i.e., sensory alteration or parasthesis including tingling); and (iv) ECOG Performance Status of 2.

### III. Therapeutic and Diagnostic Methods and Uses

A. Therapeutic methods and uses relating to cancers

Therapeutic Methods and Uses

Provided herein are methods and uses for treating cancer (e.g., a solid tumor and/or a locally advanced or metastatic cancer, e.g., a lung cancer (e.g., an early stage lung cancer (e.g., a resectable lung cancer), a small cell lung cancer (SCLC) (e.g., an extensive stage (ES)-SCLC), a non-small cell lung cancer (NSCLC) (e.g., a squamous NSCLC or a non-squamous NSCLC, a locally advanced unresectable NSCLC, a Stage IIIB NSCLC, a recurrent or metastatic NSCLC (e.g., a locally advanced unresectable or metastatic non-squamous NSCLC (e.g., Stage IV non-squamous NSCLC)), or a Stage IV NSCLC (e.g., wherein the subject has not been previously treated for Stage IV NSCLC))); a cervical cancer (e.g., a Stage IVB, metastatic, recurrent, or persistent cervical cancer, e.g., a metastatic and/or recurrent PD-L1-positive cervical carcinoma); a breast cancer (e.g., a triple-negative breast cancer (TNBC) (e.g., an estrogen receptor negative (ER−), progesterone receptor negative (PR−), and HER2 negative (HER2-) breast cancer, e.g., an early TNBC (eTNBC)) or a HER2-positive breast cancer); a head and neck cancer (e.g., squamous cell carcinoma of the head and neck (SCCHN), e.g., recurrent/metastatic PD-L1-positive SCCHN); a liver cancer (e.g., hepatocellular carcinoma (HCC), e.g., locally advanced or metastatic HCC and/or unresectable HCC); a bladder cancer (e.g., muscle-invasive bladder cancer (MIBC) or locally advanced or metastatic urothelial carcinoma (mUC)); an esophageal cancer; a pancreatic cancer (e.g., a pancreatic ductal adenocarcinoma (PDAC), e.g., a metastatic PDAC); a kidney or renal cancer (e.g., a renal cell carcinoma (RCC)); a melanoma; an ovarian cancer; a gastric cancer (e.g., a gastroesophageal junction cancer); or a colorectal cancer (CRC; e.g., CRC with microsatellite-stable (MSS) or microsatellite instability (MSI) low (MSI-Low)) in a subject comprising administering to the subject one or more dosing cycles of an effective amount of an anti-TIGIT antagonist antibody (e.g., an anti-TIGIT antagonist antibody, e.g., tiragolumab), or a combination of both an anti-TIGIT antagonist antibody and a PD-1 axis binding antagonist (e.g., an anti-PD-L1 antagonist antibody such as atezolizumab, or an anti-PD-1 antagonist antibody such as pembrolizumab).

The present invention includes methods and uses involving administration of an effective amount of an anti-TIGIT antagonist antibody (e.g., an anti-TIGIT antagonist antibody as disclosed herein, e.g., tiragolumab) and a PD-1 axis binding antagonist (e.g., an anti-PD-L1 antagonist antibody, such as atezolizumab, or an anti-PD-1 antagonist antibody, such as e.g., pembrolizumab) to a subject in need thereof every four weeks (e.g., on Day 1 of each 28-day dosing cycle). In some instances, administration of the effective amount of the anti-TIGIT antagonist antibody (e.g., an anti-TIGIT antagonist antibody as disclosed herein, e.g., tiragolumab) and the PD-1 axis binding antagonist (e.g., an anti-PD-L1 antagonist antibody, such as atezolizumab) results in a complete response (CR) or a partial response (PR). In some instances, administration of the effective amount of the anti-TIGIT antagonist antibody (e.g., an anti-TIGIT antagonist antibody as disclosed herein, e.g., tiragolumab) and the PD-1 axis binding antagonist (e.g., an anti-PD-L1 antagonist antibody, such as atezolizumab) results in an increase in progression-free survival (PFS) or duration of objective response (DOR). In some instances, administration of the effective amount of the anti-TIGIT antagonist antibody (e.g., an anti-TIGIT antagonist antibody as disclosed herein, e.g., tiragolumab) and the PD-1 axis binding antagonist (e.g., an anti-PD-L1 antagonist antibody, such as atezolizumab) results in an increase in overall survival (OS). In some instances, administration of the effective amount of the anti-TIGIT antagonist antibody (e.g., an anti-TIGIT antagonist antibody as disclosed herein, e.g., tiragolumab) and the PD-1 axis binding antagonist (e.g., an anti-PD-L1 antagonist antibody, such as atezolizumab) results in an increase in PFS of the subject, e.g., as compared to treatment with the PD-1 axis binding antagonist without the anti-TIGIT antagonist antibody or as compared to treatment with the anti-TIGIT antagonist antibody without the PD-1 axis binding antagonist. In some instances, the effective amount of the anti-TIGIT antagonist antibody (e.g., an anti-TIGIT antagonist antibody as disclosed herein, e.g., tiragolumab) and the PD-1 axis binding antagonist (e.g., an anti-PD-L1 antagonist antibody, such as atezolizumab) extends OS of the subject, e.g., as compared to treatment with the PD-1 axis binding antagonist without the anti-TIGIT antagonist antibody or as compared to treatment with the anti-TIGIT antagonist antibody without the PD-1 axis binding antagonist.

The present invention includes methods and uses involving administration of an effective amount of an anti-TIGIT antagonist antibody (e.g., an anti-TIGIT antagonist antibody as disclosed herein, e.g., tiragolumab) to a subject in need thereof every four weeks (e.g., on Day 1 of each 28-day dosing cycle). In some instances, the anti-TIGIT antagonist antibody (e.g., an anti-TIGIT antagonist antibody as disclosed herein, e.g., tiragolumab) is administered every four weeks (e.g., on Day 1 of each 28-day dosing cycle) and the PD-1 axis binding antagonist (e.g., an anti-PD-L1 antagonist antibody, e.g., atezolizumab, or an anti-PD-1 antagonist antibody, such as e.g., pembrolizumab) is administered every two weeks (e.g., on Days 1 and 15 of each 28-day dosing cycle), every three weeks (e.g., on Day 1 of each 21-day dosing cycle), or every four weeks (e.g., on Day 1 of each 28-day dosing cycle). In some instances, administration of the effective amount of the anti-TIGIT antagonist antibody (e.g., an anti-TIGIT antagonist antibody as disclosed herein, e.g., tiragolumab) results in a CR or a PR. In some instances, administration of the effective amount of the anti-TIGIT antagonist antibody (e.g., an anti-TIGIT antagonist antibody as disclosed herein, e.g., tiragolumab) results in an increase in PFS of the subject compared to a reference. In some instances, administration of the effective amount of the anti-TIGIT antagonist antibody (e.g., an anti-TIGIT antagonist antibody as disclosed herein, e.g., tiragolumab) results in an increase in DOR. In some instances, administration of the effective amount of the anti-TIGIT antagonist antibody (e.g., an anti-TIGIT antagonist antibody as disclosed herein, e.g., tiragolumab) extends OS of the subject.

The present invention includes methods and uses involving administration of an effective amount of an anti-TIGIT antagonist antibody (e.g., an anti-TIGIT antagonist antibody as disclosed herein, e.g., tiragolumab) to a subject in need thereof every two weeks (e.g., on Days 1 and 15 of each 28-day dosing cycle). In some instances, the anti-TIGIT antagonist antibody (e.g., an anti-TIGIT antagonist antibody as disclosed herein, e.g., tiragolumab) is administered every two weeks (e.g., on Days 1 and 15 of each 28-day dosing cycle) and the PD-1 axis binding antagonist (e.g., an anti-PD-L1 antagonist antibody, e.g., atezolizumab, or an anti-PD-1 antagonist antibody, such as e.g., pembrolizumab) is administered every two weeks (e.g., on Days 1 and 15 of each 28-day dosing cycle), every three weeks (e.g., on Day 1 of each 21-day dosing cycle), or every four weeks (e.g., on Day 1 of each 28-day dosing cycle). The present invention includes methods and uses involving administration of an effective amount of an anti-TIGIT antagonist antibody (e.g., an anti-TIGIT antagonist antibody as disclosed herein, e.g., tiragolumab) and a PD-1 axis binding antagonist (e.g., an anti-PD-L1 antagonist antibody, such as atezolizumab, or an anti-PD-1 antagonist antibody, such as e.g., pembrolizumab) to a subject in need thereof every two weeks (e.g., on Days 1 and 15 of each 28-day dosing cycle). In some instances, administration of the effective amount of the anti-TIGIT antagonist antibody (e.g., an anti-TIGIT antagonist antibody as disclosed herein, e.g., tiragolumab) and the PD-1 axis binding antagonist (e.g., an anti-PD-L1 antagonist antibody, such as atezolizumab) results in a CR or a PR. In some instances, administration of the effective amount of the anti-TIGIT antagonist antibody (e.g., an anti-TIGIT antagonist antibody as disclosed herein, e.g., tiragolumab) and the PD-1 axis binding antagonist (e.g., an anti-PD-L1 antagonist antibody, such as atezolizumab) results in an increase in PFS of the subject, e.g., as compared to treatment with the PD-1 axis binding antagonist without the anti-TIGIT antagonist antibody or as compared to treatment with the anti-TIGIT antagonist antibody without the PD-1 axis binding antagonist. In some instances, administration of the effective amount of the anti-TIGIT antagonist antibody (e.g., an anti-TIGIT antagonist antibody as disclosed herein, e.g., tiragolumab) and the PD-1 axis binding antagonist (e.g., an anti-PD-L1 antagonist antibody, such as atezolizumab) extends OS of the subject, e.g., as compared to treatment with the PD-1 axis binding antagonist without the anti-TIGIT antagonist antibody or as compared to treatment with the anti-TIGIT antagonist antibody without the PD-1 axis binding antagonist.

The present invention includes methods and uses involving administration of an effective amount of an anti-TIGIT antagonist antibody (e.g., an anti-TIGIT antagonist antibody as disclosed herein, e.g., tiragolumab) to a subject in need thereof every three weeks (e.g., on Day 1 of each 21-day dosing cycle). In some instances, the anti-TIGIT antagonist antibody (e.g., an anti-TIGIT antagonist antibody as disclosed herein, e.g., tiragolumab) is administered every three weeks (e.g., on Day 1 of each 21-day dosing cycle) and the PD-1 axis binding antagonist (e.g., an anti-PD-L1 antagonist antibody, e.g., atezolizumab, or an anti-PD-1 antagonist antibody, such as e.g., pembrolizumab) is administered every two weeks (e.g., on Days 1 and 15 of each 28-day dosing cycle), every three weeks (e.g., on Day 1 of each 21-day dosing cycle), or every four weeks (e.g., on Day 1 of each 28-day dosing cycle). In certain instances, the present invention includes methods and uses involving administration of an effective amount of an anti-TIGIT antagonist antibody (e.g., an anti-TIGIT antagonist antibody as disclosed herein, e.g., tiragolumab) to a subject in need thereof every three weeks (e.g., on Day 1 of each 21-day dosing cycle). In some instances, administration of the effective amount of the anti-TIGIT antagonist antibody (e.g., an anti-TIGIT antagonist antibody as disclosed herein, e.g., tiragolumab) results in a CR or a PR. In some instances, administration of the effective amount of the anti-TIGIT antagonist antibody (e.g., an anti-TIGIT antagonist antibody as disclosed herein, e.g., tiragolumab) results in an increase in PFS of the subject compared to a reference. In some instances, administration of the effective amount of the anti-TIGIT antagonist antibody (e.g., an anti-TIGIT antagonist antibody as disclosed herein, e.g., tiragolumab) extends OS of the subject. In some instances, the present invention includes a method of treating a subject having a cancer, the method comprising administering to the subject a dosing regimen comprising one or more dosing cycles of an anti-TIGIT antagonist antibody at a dose of about 500 mg to about 700 mg every three weeks, a PD-1 axis binding antagonist at a dose of about 900 mg to about 1500 mg every three weeks, a platinum-based chemotherapeutic agent every three weeks, and a non-platinum-based chemotherapeutic agent every three weeks. In some instances, the method comprises administering to the subject a dosing regimen comprising one or more dosing cycles of an anti-TIGIT antagonist antibody at a dose of 500 mg to 700 mg every three weeks, a PD-1 axis binding antagonist at a dose of 900 mg to 1500 mg every three weeks, a platinum-based chemotherapeutic agent every three weeks, and a non-platinum-based chemotherapeutic agent every three weeks.

In certain instances, the PD-1 axis binding antagonist and the anti-TIGIT antagonist antibody are administered without a chemotherapeutic agent (e.g., without any chemotherapeutic agent, e.g., the entire dosing regimen is devoid of administration of a chemotherapeutic agent to the subject).

In some embodiments, the subject has not been previously treated with a therapy (e.g., a cancer immunotherapy and/or a chemotherapeutic agent) for the cancer. In some embodiments, the subject has received prior treatment with a therapy (e.g., a cancer immunotherapy and/or a chemotherapeutic agent) for the cancer. In some instances, the subject has not received prior systemic therapy (e.g., e.g., prior systemic therapy with curative intent, e.g., chemotherapy) within at least the month prior to the administration with the PD-1 axis binding antagonist and the anti-TIGIT antagonist antibody (e.g., within the two months prior, three months prior, four months prior, six months prior, one year prior, two years prior, three years prior, four years prior, five years prior, or ten years prior to the administration with the PD-1 axis binding antagonist and the anti-TIGIT antagonist antibody). In some instances, the subject is chemotherapy naïve.

In some embodiments, the PD-1 axis binding antagonist and the anti-TIGIT antagonist antibody are administered in conjunction with a chemotherapy. For example, a once-every-two-weeks (Q2W), once-every-three-weeks (Q3W), or once-every-four-weeks (Q4W) dosing regimen of the PD-1 axis binding antagonist and/or the anti-TIGIT antagonist antibody can be administered in conjunction with one or more chemotherapeutic agents. The one or more chemotherapeutic agents can be administered at the same frequency as the frequency of administration of the PD-1 axis binding antagonist and the anti-TIGIT antagonist antibody (Q2W, Q3W, or Q4W) or at a different frequency (e.g., 3-weeks on/1-week off schedule (e.g., Days 1, 8, and 15 of every 28-day cycle)). For example, in some embodiments, the PD-1 axis binding antagonist and the anti-TIGIT antagonist antibody are administered every two weeks and the one or more chemotherapeutic agents is administered every week, 3-weeks on/1-week off, every two weeks, every three weeks, or every four weeks. Alternatively, the PD-1 axis binding antagonist and the anti-TIGIT antagonist antibody are administered every three weeks and the one or more chemotherapeutic agents is administered every week, two weeks, every three weeks, or every four weeks. Alternatively, the PD-1 axis binding antagonist and the anti-TIGIT antagonist antibody are administered every four weeks and the one or more chemotherapeutic agents is administered every week, 3-weeks on/1-week off, every two weeks, every three weeks, or every four weeks. In certain instances, a chemotherapeutic agent is administered multiple times per week (e.g., 2, 3, 4, 5, 6 or 7 times per week (e.g., at Days 1, 2, and 3 of a dosing cycle).

In some embodiments, the dose of a chemotherapeutic agent is reduced after one or more initial doses (e.g., after one, two, three, four, or more initial doses). For example, a subsequent dose of the chemotherapeutic agent (e.g., a platinum-based chemotherapeutic agent (e.g., carboplatin or cisplatin) and/or one or more non-platinum-based chemotherapeutic agents (e.g., an alkylating agent (e.g., cyclophosphamide), a taxane (e.g., paclitaxel, e.g., nab-paclitaxel), and/or a topoisomerase II inhibitor (e.g., doxorubicin))) can be administered at about 95%, 90%, 85%, 80%, 75%, 70%, 65%, 60%, 55%, 50%, 45%, 40%, 35%, 30%, 25%, 20%, 15%, 10%, or 5% of the initial dose. For example, an initial dose of nab-paclitaxel of 125 mg/m² can be reduced for a subsequent dose, e.g., to 100 mg/m² or 75 mg/m²; an initial dose of nab-paclitaxel of 100 mg/m² can be reduced for a subsequent dose, e.g., to 75 mg/m²; an initial dose of paclitaxel of about 175 mg/m² can be reduced for a subsequent dose, e.g., to 150 mg/m², 125 mg/m², 100 mg/m², or 75 mg/m²; an initial dose of paclitaxel of about 200 mg/m² can be reduced for a subsequent dose, e.g., to 175 mg/m², 150 mg/m², 125 mg/m², 100 mg/m², or 75 mg/m²; an initial dose of gemcitabine of about 1000 mg/m² can be reduced for a subsequent dose, e.g., to 900 mg/m², 800 mg/m², 750 mg/m², 700 mg/m², 600 mg/m², or 500 mg/m²; an initial dose of cisplatin of about 75 mg/m² can be reduced for a subsequent dose, e.g., to 70 mg/m², 65 mg/m², 60 mg/m², 55 mg/m², 50 mg/m², or 45 mg/m²; an initial dose of pemetrexed of about 500 mg/m² can be reduced for a subsequent dose, e.g., to 450 mg/m², 400 mg/m², 350 mg/m², 300 mg/m², 250 mg/m², or 200 mg/m²; and/or an initial dose of carboplatin of a dose sufficient to achieve AUC=6 mg/ml/min can be reduced for a subsequent dose, e.g., to a dose sufficient to achieve AUC=5.5. mg/ml/min, 5.0 mg/ml/min, 4.5 mg/ml/min, or 4.0 mg/ml/min. In some examples, an initial dose of nab-paclitaxel of 125 mg/m² can be reduced for a subsequent dose, e.g., to 100 mg/m² or 75 mg/m²; an initial dose of nab-paclitaxel of 100 mg/m² can be reduced for a subsequent dose, e.g., to 75 mg/m²; an initial dose of paclitaxel of 175 mg/m² can be reduced for a subsequent dose, e.g., to 150 mg/m², 125 mg/m², 100 mg/m², or 75 mg/m²; an initial dose of paclitaxel of 200 mg/m² can be reduced for a subsequent dose, e.g., to 175 mg/m², 150 mg/m², 125 mg/m², 100 mg/m², or 75 mg/m²; an initial dose of gemcitabine of 1000 mg/m² can be reduced for a subsequent dose, e.g., to 900 mg/m², 800 mg/m², 750 mg/m², 700 mg/m², 600 mg/m², or 500 mg/m²; an initial dose of cisplatin of 75 mg/m² can be reduced for a subsequent dose, e.g., to 70 mg/m², 65 mg/m², 60 mg/m², 55 mg/m², 50 mg/m², or 45 mg/m²; an initial dose of pemetrexed of 500 mg/m² can be reduced for a subsequent dose, e.g., to 450 mg/m², 400 mg/m², 350 mg/m², 300 mg/m², 250 mg/m², or 200 mg/m²; and/or an initial dose of carboplatin of a dose sufficient to achieve AUC=6 mg/ml/min can be reduced for a subsequent dose, e.g., to a dose sufficient to achieve AUC=5.5. mg/ml/min, 5.0 mg/ml/min, 4.5 mg/ml/min, or 4.0 mg/ml/min.

The present invention includes methods and uses involving administration of an effective amount of an anti-TIGIT antagonist antibody (e.g., an anti-TIGIT antagonist antibody as disclosed herein, e.g., tiragolumab), a PD-1 axis binding antagonist (e.g., an anti-PD-L1 antagonist antibody, such as atezolizumab, or an anti-PD-1 antagonist antibody such as pembrolizumab), and a chemotherapy combination to a subject in need thereof. In some embodiments, the anti-TIGIT antagonist antibody (e.g., anti-TIGIT antagonist antibody as disclosed herein, e.g., tiragolumab) and the PD-1 axis binding antagonist (e.g., an anti-PD-L1 antagonist antibody, e.g., atezolizumab) are administered every two weeks (e.g., on Days 1 and 15 of each 28-day dosing cycle), every three weeks (e.g., on Day 1 of each 21-day dosing cycle), or every four weeks (e.g., on Day 1 of each 28-day dosing cycle). In some instances, the anti-TIGIT antagonist antibody (e.g., an anti-TIGIT antagonist antibody as disclosed herein, e.g., tiragolumab) is administered every four weeks (e.g., on Day 1 of each 28-day dosing cycle) and the PD-1 axis binding antagonist (e.g., an anti-PD-L1 antagonist antibody, e.g., atezolizumab) is administered every two weeks (e.g., on Days 1 and 15 of each 28-day dosing cycle), every three weeks (e.g., on Day 1 of each 21-day dosing cycle), or every four weeks (e.g., on Day 1 of each 28-day dosing cycle). In some instances, the chemotherapy combination includes an effective amount of a first non-platinum-based chemotherapeutic agent and an effective amount of a second non-platinum-based chemotherapeutic agent. In some instances, the first non-platinum-based chemotherapeutic agent is an antimetabolite and the second non-platinum-based chemotherapeutic agent is a taxane. In some embodiments, the chemotherapy combination (e.g., the antimetabolite (e.g., gemcitabine, pemetrexed, or capecitabine) and the taxane (e.g., nab-paclitaxel and paclitaxel)) is administered weekly, biweekly, or three times every four weeks (e.g., on Days 1, 8, and 15 of each 28-day dosing cycle).

In particular embodiments, the method involves administration of an effective amount of an anti-TIGIT antagonist antibody (e.g., an anti-TIGIT antagonist antibody as disclosed herein, e.g., tiragolumab), a PD-1 axis binding antagonist (e.g., an anti-PD-L1 antagonist antibody, e.g., atezolizumab), gemcitabine, and paclitaxel to a subject in need thereof, wherein the anti-TIGIT antagonist antibody (e.g., anti-TIGIT antagonist antibody as disclosed herein, e.g., tiragolumab) and the PD-1 axis binding antagonist (e.g., an anti-PD-L1 antagonist antibody, e.g., atezolizumab) are administered every two weeks (e.g., on Days 1 and 15 of each 28-day dosing cycle) and the chemotherapy combination (e.g., the antimetabolite and the taxane (e.g., gemcitabine and paclitaxel)) is administered more frequently (e.g., three times every four weeks (e.g., on Days 1, 8, and 15 of each 28-day dosing cycle).

In some instances, administration of the effective amount of the anti-TIGIT antagonist antibody (e.g., an anti-TIGIT antagonist antibody as disclosed herein, e.g., tiragolumab), the PD-1 axis binding antagonist (e.g., anti-PD-L1 antagonist antibody, such as atezolizumab), the antimetabolite (e.g., gemcitabine), and the taxane (e.g., paclitaxel) results in a CR or a PR. In some instances, administration of the effective amount of the anti-TIGIT antagonist antibody (e.g., an anti-TIGIT antagonist antibody as disclosed herein, e.g., tiragolumab), the PD-1 axis binding antagonist (e.g., anti-PD-L1 antagonist antibody, such as atezolizumab), the antimetabolite (e.g., gemcitabine), and the taxane (e.g., paclitaxel) results in an increase in PFS of the subject. In some instances, administration of the effective amount of the anti-TIGIT antagonist antibody (e.g., an anti-TIGIT antagonist antibody as disclosed herein, e.g., tiragolumab), the PD-1 axis binding antagonist (e.g., anti-PD-L1 antagonist antibody, such as atezolizumab), the antimetabolite (e.g., gemcitabine), and the taxane (e.g., paclitaxel) extends OS of the subject.

The present invention includes methods and uses involving administration of an effective amount of an anti-TIGIT antagonist antibody (e.g., an anti-TIGIT antagonist antibody as disclosed herein, e.g., tiragolumab), a PD-1 axis binding antagonist (e.g., an anti-PD-L1 antagonist antibody, such as atezolizumab, or an anti-PD-1 antagonist antibody such as pembrolizumab), and a chemotherapy combination to a subject in need thereof, wherein the chemotherapy combination includes an effective amount of a platinum-based chemotherapeutic agent and an effective amount of a non-platinum-based chemotherapeutic agent. In some instances, the anti-TIGIT antagonist antibody (e.g., anti-TIGIT antagonist antibody as disclosed herein, e.g., tiragolumab) and the PD-1 axis binding antagonist (e.g., an anti-PD-L1 antagonist antibody, e.g., atezolizumab) are administered every two weeks (e.g., on Days 1 and 15 of each 28-day dosing cycle), every three weeks (e.g., on Day 1 of each 21-day dosing cycle), or every four weeks (e.g., on Day 1 of each 28-day dosing cycle). In some instances, the platinum-based chemotherapeutic agent is carboplatin or cisplatin and the non-platinum-based chemotherapeutic agent is an antimetabolite (e.g., pemetrexed). In some embodiments, the chemotherapy combination (e.g., the platinum-based chemotherapeutic agent and the antimetabolite (e.g., pemetrexed)) are administered weekly, every two weeks, every four weeks, or three times every four weeks (e.g., on Days 1, 8, and 15 of each 28-day dosing cycle).

In particular embodiments, the method involves administration of an effective amount of an anti-TIGIT antagonist antibody (e.g., an anti-TIGIT antagonist antibody as disclosed herein, e.g., tiragolumab), a PD-1 axis binding antagonist (e.g., an anti-PD-L1 antagonist antibody, e.g., atezolizumab), a platinum-based chemotherapeutic agent (e.g., carboplatin or cisplatin), and an antimetabolite (e.g., pemetrexed) to a subject in need thereof, wherein the anti-TIGIT antagonist antibody (e.g., anti-TIGIT antagonist antibody as disclosed herein, e.g., tiragolumab) and the PD-1 axis binding antagonist (e.g., an anti-PD-L1 antagonist antibody, e.g., atezolizumab) are administered every three weeks (e.g., on Day 1 of each 21-day dosing cycle) and the chemotherapy combination (e.g., the platinum-based chemotherapeutic agent (e.g., carboplatin or cisplatin) and the antimetabolite (e.g., pemetrexed) are administered at the same frequency (e.g., every three weeks, e.g., on Day 1 of each 21-day dosing cycle). In some instances, the dosing continues for four-to-six induction dosing cycles (e.g., four induction dosing cycles, five induction dosing cycles, or six induction dosing cycles). After the induction dosing cycles, maintenance therapy can be administered in one or more subsequent (maintenance) dosing cycles. In certain embodiments, the one or more maintenance dosing cycles does not include the platinum-based chemotherapeutic agent.

In some instances, administration of the effective amount of the anti-TIGIT antagonist antibody (e.g., an anti-TIGIT antagonist antibody as disclosed herein, e.g., tiragolumab), the PD-1 axis binding antagonist (e.g., anti-PD-L1 antagonist antibody, such as atezolizumab), the platinum-based chemotherapeutic agent (e.g., carboplatin or cisplatin), and the antimetabolite (e.g., pemetrexed) results in a CR or a PR. In some instances, administration of the effective amount of the anti-TIGIT antagonist antibody (e.g., an anti-TIGIT antagonist antibody as disclosed herein, e.g., tiragolumab), the PD-1 axis binding antagonist (e.g., anti-PD-L1 antagonist antibody, such as atezolizumab), the platinum-based chemotherapeutic agent (e.g., carboplatin or cisplatin), and the antimetabolite (e.g., pemetrexed) results in an increase in PFS of the subject. In some instances, administration of the effective amount of the anti-TIGIT antagonist antibody (e.g., an anti-TIGIT antagonist antibody as disclosed herein, e.g., tiragolumab), the PD-1 axis binding antagonist (e.g., anti-PD-L1 antagonist antibody, such as atezolizumab), the platinum-based chemotherapeutic agent (e.g., carboplatin or cisplatin), and the antimetabolite (e.g., pemetrexed) extends OS of the subject.

In some instances, the subject receiving the anti-TIGIT antagonist antibody (e.g., an anti-TIGIT antagonist antibody as disclosed herein, e.g., tiragolumab), the PD-1 axis binding antagonist (e.g., anti-PD-L1 antagonist antibody, such as atezolizumab), the platinum-based chemotherapeutic agent (e.g., carboplatin or cisplatin), and the antimetabolite (e.g., pemetrexed) is being treated for a solid tumor or a locally advanced or metastatic cancer. Additionally or alternatively, the cancer may be a lung cancer (e.g., an early stage lung cancer (e.g., a resectable lung cancer), a SCLC (e.g., an ES-SCLC), a NSCLC (e.g., a squamous NSCLC or a non-squamous NSCLC, a locally advanced unresectable NSCLC, a Stage IIIB NSCLC, a recurrent or metastatic NSCLC (e.g., a locally advanced unresectable or metastatic non-squamous NSCLC (e.g., Stage IV non-squamous NSCLC)), or a Stage IV NSCLC (e.g., wherein the subject has not been previously treated for Stage IV NSCLC))); a cervical cancer (e.g., a Stage IVB, metastatic, recurrent, or persistent cervical cancer, e.g., a metastatic and/or recurrent PD-L1-positive cervical carcinoma); a breast cancer (e.g., a TNBC (e.g., an early TNBC (eTNBC))) or a HER2-positive breast cancer); a head and neck cancer (e.g., SCCHN, e.g., recurrent/metastatic PD-L1-positive SCCHN); a liver cancer (e.g., HCC, e.g., locally advanced or metastatic HCC and/or unresectable HCC); a bladder cancer (e.g., MIBC, locally advanced UC, or mUC); an esophageal cancer; a pancreatic cancer (e.g., PDAC, e.g., metastatic PDAC); a kidney or renal cancer (e.g., a RCC); a melanoma; an ovarian cancer; a gastric cancer (e.g., a gastroesophageal junction cancer); or a CRC (e.g., MSS or MSI-Low CRC).

The present invention also includes methods and uses involving administration of an effective amount of an anti-TIGIT antagonist antibody (e.g., an anti-TIGIT antagonist antibody as disclosed herein, e.g., tiragolumab), a PD-1 axis binding antagonist (e.g., an anti-PD-L1 antagonist antibody, such as atezolizumab, or an anti-PD-1 antagonist antibody such as pembrolizumab), and a chemotherapy (e.g., a taxane (e.g., paclitaxel or nab-paclitaxel)) to a subject in need thereof. In some instances, the anti-TIGIT antagonist antibody (e.g., anti-TIGIT antagonist antibody as disclosed herein, e.g., tiragolumab) and the PD-1 axis binding antagonist (e.g., an anti-PD-L1 antagonist antibody, e.g., atezolizumab) are administered every two weeks (e.g., on Days 1 and 15 of each 28-day dosing cycle), every three weeks (e.g., on Day 1 of each 21-day dosing cycle), or every four weeks (e.g., on Day 1 of each 28-day dosing cycle). In some instances, the anti-TIGIT antagonist antibody (e.g., anti-TIGIT antagonist antibody as disclosed herein, e.g., tiragolumab) and the PD-1 axis binding antagonist (e.g., an anti-PD-L1 antagonist antibody, e.g., atezolizumab) are administered every four weeks (e.g., on Day 1 of each 28-day dosing cycle). In some embodiments, the chemotherapy is administered weekly, every two weeks, every four weeks, or three times every four weeks (e.g., on Days 1, 8, and 15 of each 28-day dosing cycle). In some embodiments, the chemotherapy is administered weekly.

In some instances, administration of an effective amount of an anti-TIGIT antagonist antibody (e.g., an anti-TIGIT antagonist antibody as disclosed herein, e.g., tiragolumab), a PD-1 axis binding antagonist (e.g., an anti-PD-L1 antagonist antibody, such as atezolizumab, or an anti-PD-1 antagonist antibody such as pembrolizumab), and a chemotherapy (e.g., a taxane (e.g., paclitaxel or nab-paclitaxel)) results in a CR or a PR. In some instances, administration of an effective amount of an anti-TIGIT antagonist antibody (e.g., an anti-TIGIT antagonist antibody as disclosed herein, e.g., tiragolumab), a PD-1 axis binding antagonist (e.g., an anti-PD-L1 antagonist antibody, such as atezolizumab, or an anti-PD-1 antagonist antibody such as pembrolizumab), and a chemotherapy (e.g., a taxane (e.g., paclitaxel or nab-paclitaxel)) results in an increase in PFS of the subject compared to a reference. In some instances, administration of an effective amount of an anti-TIGIT antagonist antibody (e.g., an anti-TIGIT antagonist antibody as disclosed herein, e.g., tiragolumab), a PD-1 axis binding antagonist (e.g., an anti-PD-L1 antagonist antibody, such as atezolizumab, or an anti-PD-1 antagonist antibody such as pembrolizumab), and a chemotherapy (e.g., a taxane (e.g., paclitaxel or nab-paclitaxel)) results in an increase in DOR. In some instances, administration of an effective amount of an anti-TIGIT antagonist antibody (e.g., an anti-TIGIT antagonist antibody as disclosed herein, e.g., tiragolumab), a PD-1 axis binding antagonist (e.g., an anti-PD-L1 antagonist antibody, such as atezolizumab, or an anti-PD-1 antagonist antibody such as pembrolizumab), and a chemotherapy (e.g., a taxane (e.g., paclitaxel or nab-paclitaxel)) extends OS of the subject.

The present invention also includes methods and uses involving administration of an effective amount of an anti-TIGIT antagonist antibody (e.g., an anti-TIGIT antagonist antibody as disclosed herein, e.g., tiragolumab), a PD-1 axis binding antagonist (e.g., an anti-PD-L1 antagonist antibody, such as atezolizumab, or an anti-PD-1 antagonist antibody such as pembrolizumab), and a chemotherapy combination to a subject in need thereof, wherein the chemotherapy combination includes an effective amount of a platinum-based chemotherapeutic agent and an effective amount of a non-platinum-based chemotherapeutic agent, wherein the non-platinum-based chemotherapeutic agent is a taxane (e.g., paclitaxel or nab-paclitaxel). In some instances, the anti-TIGIT antagonist antibody (e.g., anti-TIGIT antagonist antibody as disclosed herein, e.g., tiragolumab) and the PD-1 axis binding antagonist (e.g., an anti-PD-L1 antagonist antibody, e.g., atezolizumab) are administered every two weeks (e.g., on Days 1 and 15 of each 28-day dosing cycle), every three weeks (e.g., on Day 1 of each 21-day dosing cycle), or every four weeks (e.g., on Day 1 of each 28-day dosing cycle). In some embodiments, the chemotherapy combination (e.g., the platinum-based chemotherapeutic agent and the taxane (e.g., paclitaxel or nab-paclitaxel)) are administered weekly, every two weeks, every four weeks, or three times every four weeks (e.g., on Days 1, 8, and 15 of each 28-day dosing cycle).

In particular embodiments, the method involves administration of an effective amount of an anti-TIGIT antagonist antibody (e.g., an anti-TIGIT antagonist antibody as disclosed herein, e.g., tiragolumab), a PD-1 axis binding antagonist (e.g., an anti-PD-L1 antagonist antibody, e.g., atezolizumab), a platinum-based chemotherapeutic agent (e.g., carboplatin or cisplatin), and a taxane (e.g., paclitaxel or nab-paclitaxel) to a subject in need thereof, wherein the anti-TIGIT antagonist antibody (e.g., anti-TIGIT antagonist antibody as disclosed herein, e.g., tiragolumab) and the PD-1 axis binding antagonist (e.g., an anti-PD-L1 antagonist antibody, e.g., atezolizumab) are administered every three weeks (e.g., on Day 1 of each 21-day dosing cycle) and the chemotherapy combination (e.g., the platinum-based chemotherapeutic agent (e.g., carboplatin or cisplatin) and the taxane (e.g., paclitaxel or nab-paclitaxel) are administered at the same frequency (e.g., every three weeks, e.g., on Day 1 of each 21-day dosing cycle). In some instances, the dosing continues for four-to-six induction dosing cycles (e.g., four induction dosing cycles, five induction dosing cycles, or six induction dosing cycles). After the induction dosing cycles, maintenance therapy can be administered in one or more subsequent (maintenance) dosing cycles. In certain embodiments, the one or more maintenance dosing cycles does not include the platinum-based chemotherapeutic agent.

In some instances, administration of the effective amount of the anti-TIGIT antagonist antibody (e.g., an anti-TIGIT antagonist antibody as disclosed herein, e.g., tiragolumab), the PD-1 axis binding antagonist (e.g., anti-PD-L1 antagonist antibody, such as atezolizumab), the platinum-based chemotherapeutic agent (e.g., carboplatin or cisplatin), and the taxane (e.g., paclitaxel or nab-paclitaxel) results in a CR or a PR. In some instances, administration of the effective amount of the anti-TIGIT antagonist antibody (e.g., an anti-TIGIT antagonist antibody as disclosed herein, e.g., tiragolumab), the PD-1 axis binding antagonist (e.g., anti-PD-L1 antagonist antibody, such as atezolizumab), the platinum-based chemotherapeutic agent (e.g., carboplatin or cisplatin), and the taxane (e.g., paclitaxel or nab-paclitaxel) results in an increase in PFS of the subject. In some instances, administration of the effective amount of the anti-TIGIT antagonist antibody (e.g., an anti-TIGIT antagonist antibody as disclosed herein, e.g., tiragolumab), the PD-1 axis binding antagonist (e.g., anti-PD-L1 antagonist antibody, such as atezolizumab), the platinum-based chemotherapeutic agent (e.g., carboplatin or cisplatin), and the taxane (e.g., paclitaxel or nab-paclitaxel) extends OS of the subject.

In some instances, the subject receiving the anti-TIGIT antagonist antibody (e.g., an anti-TIGIT antagonist antibody as disclosed herein, e.g., tiragolumab), the PD-1 axis binding antagonist (e.g., anti-PD-L1 antagonist antibody, such as atezolizumab), the platinum-based chemotherapeutic agent (e.g., carboplatin or cisplatin), and the taxane (e.g., paclitaxel or nab-paclitaxel) is being treated for a solid tumor or a locally advanced or metastatic cancer. Additionally or alternatively, the cancer may be a lung cancer (e.g., an early stage lung cancer (e.g., a resectable lung cancer), a SCLC (e.g., an ES-SCLC), a NSCLC (e.g., a squamous NSCLC or a non-squamous NSCLC, a locally advanced unresectable NSCLC, a Stage IIIB NSCLC, a recurrent or metastatic NSCLC (e.g., a locally advanced unresectable or metastatic non-squamous NSCLC (e.g., Stage IV non-squamous NSCLC)), or a Stage IV NSCLC (e.g., wherein the subject has not been previously treated for Stage IV NSCLC))); a cervical cancer (e.g., a Stage IVB, metastatic, recurrent, or persistent cervical cancer, e.g., a metastatic and/or recurrent PD-L1-positive cervical carcinoma); a breast cancer (e.g., a TNBC (e.g., an early TNBC (eTNBC))) or a HER2-positive breast cancer); a head and neck cancer (e.g., SCCHN, e.g., recurrent/metastatic PD-L1-positive SCCHN); a liver cancer (e.g., HCC, e.g., locally advanced or metastatic HCC and/or unresectable HCC); a bladder cancer (e.g., MIBC, locally advanced UC, or mUC); an esophageal cancer; a pancreatic cancer (e.g., PDAC, e.g., metastatic PDAC); a kidney or renal cancer (e.g., a RCC); a melanoma; an ovarian cancer; a gastric cancer (e.g., a gastroesophageal junction cancer); or a CRC (e.g., MSS or MSI-Low CRC).

Also provided herein are methods and uses involving administration of an effective amount of an anti-TIGIT antagonist antibody (e.g., an anti-TIGIT antagonist antibody as disclosed herein, e.g., tiragolumab), a PD-1 axis binding antagonist (e.g., an anti-PD-L1 antagonist antibody, such as atezolizumab, or an anti-PD-1 antagonist antibody such as pembrolizumab), and a chemotherapy combination to a subject in need thereof, wherein the chemotherapy combination includes an effective amount of a platinum-based chemotherapeutic agent and an effective amount of a non-platinum-based chemotherapeutic agent, wherein the non-platinum-based chemotherapeutic agent is a topoisomerase II inhibitor (e.g., etoposide). In some instances, the anti-TIGIT antagonist antibody (e.g., anti-TIGIT antagonist antibody as disclosed herein, e.g., tiragolumab) and the PD-1 axis binding antagonist (e.g., an anti-PD-L1 antagonist antibody, e.g., atezolizumab) are administered every two weeks (e.g., on Days 1 and 15 of each 28-day dosing cycle), every three weeks (e.g., on Day 1 of each 21-day dosing cycle), or every four weeks (e.g., on Day 1 of each 28-day dosing cycle). In some embodiments, the chemotherapy combination (e.g., the platinum-based chemotherapeutic agent and the topoisomerase II inhibitor (e.g., etoposide)) are administered weekly, every two weeks, every four weeks, or three times every four weeks (e.g., on Days 1, 8, and 15 of each 28-day dosing cycle). In some embodiments, the topoisomerase II inhibitor (e.g., etoposide) is administered more frequently than the platinum-based chemotherapeutic agent (e.g., three times per week, e.g., on Days 1, 2, and 3 of each dosing cycle).

In particular embodiments, the method involves administration of an effective amount of an anti-TIGIT antagonist antibody (e.g., an anti-TIGIT antagonist antibody as disclosed herein, e.g., tiragolumab), a PD-1 axis binding antagonist (e.g., an anti-PD-L1 antagonist antibody, e.g., atezolizumab), a platinum-based chemotherapeutic agent (e.g., carboplatin or cisplatin), and a topoisomerase II inhibitor (e.g., etoposide) to a subject in need thereof, wherein the anti-TIGIT antagonist antibody (e.g., anti-TIGIT antagonist antibody as disclosed herein, e.g., tiragolumab) and the PD-1 axis binding antagonist (e.g., an anti-PD-L1 antagonist antibody, e.g., atezolizumab) are administered every three weeks (e.g., on Day 1 of each 21-day dosing cycle) and the chemotherapy combination (e.g., the platinum-based chemotherapeutic agent (e.g., carboplatin or cisplatin) and the topoisomerase II inhibitor (e.g., etoposide) are administered at the same frequency (e.g., every three weeks, e.g., on Day 1 of each 21-day dosing cycle). In some instances, the dosing continues for four-to-six induction dosing cycles (e.g., four induction dosing cycles, five induction dosing cycles, or six induction dosing cycles). After the induction dosing cycles, maintenance therapy can be administered in one or more subsequent (maintenance) dosing cycles. In certain embodiments, the one or more maintenance dosing cycles does not include the platinum-based chemotherapeutic agent or the topoisomerase II inhibitor (e.g., etoposide).

In some instances, the effective amount of the anti-TIGIT antagonist antibody (e.g., an anti-TIGIT antagonist antibody as disclosed herein, e.g., tiragolumab), the PD-1 axis binding antagonist (e.g., anti-PD-L1 antagonist antibody, such as atezolizumab), the platinum-based chemotherapeutic agent (e.g., carboplatin or cisplatin), and the topoisomerase II inhibitor (e.g., etoposide) results in (a) a CR or a PR. In some instances, the effective amount of the anti-TIGIT antagonist antibody (e.g., an anti-TIGIT antagonist antibody as disclosed herein, e.g., tiragolumab), the PD-1 axis binding antagonist (e.g., anti-PD-L1 antagonist antibody, such as atezolizumab), the platinum-based chemotherapeutic agent (e.g., carboplatin or cisplatin), and the topoisomerase II inhibitor (e.g., etoposide) results in an increase in PFS of the subject. In some instances, the effective amount of the anti-TIGIT antagonist antibody (e.g., an anti-TIGIT antagonist antibody as disclosed herein, e.g., tiragolumab), the PD-1 axis binding antagonist (e.g., anti-PD-L1 antagonist antibody, such as atezolizumab), the platinum-based chemotherapeutic agent (e.g., carboplatin or cisplatin), and the topoisomerase II inhibitor (e.g., etoposide) extends OS of the subject.

In some instances, the subject receiving the anti-TIGIT antagonist antibody (e.g., an anti-TIGIT antagonist antibody as disclosed herein, e.g., tiragolumab), the PD-1 axis binding antagonist (e.g., anti-PD-L1 antagonist antibody, such as atezolizumab), the platinum-based chemotherapeutic agent (e.g., carboplatin or cisplatin), and the topoisomerase II inhibitor (e.g., etoposide) is being treated for a solid tumor or a locally advanced or metastatic cancer. Additionally or alternatively, the cancer may be a lung cancer (e.g., an early stage lung cancer (e.g., a resectable lung cancer), a SCLC (e.g., an ES-SCLC), a NSCLC (e.g., a squamous NSCLC or a non-squamous NSCLC, a locally advanced unresectable NSCLC, a Stage IIIB NSCLC, a recurrent or metastatic NSCLC (e.g., a locally advanced unresectable or metastatic non-squamous NSCLC (e.g., Stage IV non-squamous NSCLC)), or a Stage IV NSCLC (e.g., wherein the subject has not been previously treated for Stage IV NSCLC))); a cervical cancer (e.g., a Stage IVB, metastatic, recurrent, or persistent cervical cancer, e.g., a metastatic and/or recurrent PD-L1-positive cervical carcinoma); a breast cancer (e.g., a TNBC (e.g., an early TNBC (eTNBC))) or a HER2-positive breast cancer); a head and neck cancer (e.g., SCCHN, e.g., recurrent/metastatic PD-L1-positive SCCHN); a liver cancer (e.g., HCC, e.g., locally advanced or metastatic HCC and/or unresectable HCC); a bladder cancer (e.g., MIBC, locally advanced UC, or mUC); an esophageal cancer; a pancreatic cancer (e.g., PDAC, e.g., metastatic PDAC); a kidney or renal cancer (e.g., a RCC); a melanoma; an ovarian cancer; a gastric cancer (e.g., a gastroesophageal junction cancer); or a CRC (e.g., MSS or MSI-Low CRC).

Also provided herein are methods and uses involving administration of an effective amount of an anti-TIGIT antagonist antibody (e.g., an anti-TIGIT antagonist antibody as disclosed herein, e.g., tiragolumab), a PD-1 axis binding antagonist (e.g., an anti-PD-L1 antagonist antibody, such as atezolizumab, or an anti-PD-1 antagonist antibody such as pembrolizumab), and a VEGF antagonist (e.g., an anti-VEGF antibody (e.g., bevacizumab)) to a subject in need thereof. In some instances, the anti-TIGIT antagonist antibody (e.g., anti-TIGIT antagonist antibody as disclosed herein, e.g., tiragolumab), the PD-1 axis binding antagonist (e.g., an anti-PD-L1 antagonist antibody, e.g., atezolizumab), and the VEGF antagonist (e.g., an anti-VEGF antibody (e.g., bevacizumab)) are administered every three weeks (e.g., on Day 1 of each 21-day dosing cycle).

In particular embodiments, the method involves administration of an effective amount of an anti-TIGIT antagonist antibody (e.g., an anti-TIGIT antagonist antibody as disclosed herein, e.g., tiragolumab), a PD-1 axis binding antagonist (e.g., an anti-PD-L1 antagonist antibody, e.g., atezolizumab), and the VEGF antagonist (e.g., an anti-VEGF antibody (e.g., bevacizumab)) to a subject in need thereof, wherein the anti-TIGIT antagonist antibody (e.g., anti-TIGIT antagonist antibody as disclosed herein, e.g., tiragolumab), the PD-1 axis binding antagonist (e.g., an anti-PD-L1 antagonist antibody, e.g., atezolizumab), and the VEGF antagonist (e.g., an anti-VEGF antibody (e.g., bevacizumab)) are administered every three weeks (e.g., on Day 1 of each 21-day dosing cycle). In some instances, the effective amount of the anti-TIGIT antagonist antibody (e.g., an anti-TIGIT antagonist antibody as disclosed herein, e.g., tiragolumab), the PD-1 axis binding antagonist (e.g., anti-PD-L1 antagonist antibody, such as atezolizumab), and the VEGF antagonist (e.g., an anti-VEGF antibody (e.g., bevacizumab)) results in an increase in OS of the subject. In some instances, the effective amount of the anti-TIGIT antagonist antibody (e.g., an anti-TIGIT antagonist antibody as disclosed herein, e.g., tiragolumab), the PD-1 axis binding antagonist (e.g., anti-PD-L1 antagonist antibody, such as atezolizumab), and the VEGF antagonist (e.g., an anti-VEGF antibody (e.g., bevacizumab)) results in an increase in PFS of the subject.

In some instances, the subject receiving the anti-TIGIT antagonist antibody (e.g., an anti-TIGIT antagonist antibody as disclosed herein, e.g., tiragolumab), the PD-1 axis binding antagonist (e.g., anti-PD-L1 antagonist antibody, such as atezolizumab), and the VEGF antagonist (e.g., an anti-VEGF antibody (e.g., bevacizumab)) is being treated for a solid tumor or a locally advanced or metastatic cancer. Additionally or alternatively, the cancer may be a lung cancer (e.g., an early stage lung cancer (e.g., a resectable lung cancer), a SCLC (e.g., an ES-SCLC), a NSCLC (e.g., a squamous NSCLC or a non-squamous NSCLC, a locally advanced unresectable NSCLC, a Stage IIIB NSCLC, a recurrent or metastatic NSCLC (e.g., a locally advanced unresectable or metastatic non-squamous NSCLC (e.g., Stage IV non-squamous NSCLC)), or a Stage IV NSCLC (e.g., wherein the subject has not been previously treated for Stage IV NSCLC))); a cervical cancer (e.g., a Stage IVB, metastatic, recurrent, or persistent cervical cancer, e.g., a metastatic and/or recurrent PD-L1-positive cervical carcinoma); a breast cancer (e.g., a TNBC (e.g., an eTNBC)) or a HER2-positive breast cancer); a head and neck cancer (e.g., SCCHN, e.g., recurrent/metastatic PD-L1-positive SCCHN); a liver cancer (e.g., HCC, e.g., locally advanced or metastatic HCC and/or unresectable HCC); a bladder cancer (e.g., MIBC, locally advanced UC, or mUC); an esophageal cancer; a pancreatic cancer (e.g., PDAC, e.g., metastatic PDAC); a kidney or renal cancer (e.g., a RCC); a melanoma; an ovarian cancer; a gastric cancer (e.g., a gastroesophageal junction cancer); or a CRC (e.g., MSS or MSI-Low CRC).

The present invention includes a method of treating cancer in a cancer patient comprising administering to the patient a combination of atezolizumab, bevacizumab, and tiragolumab in an amount effective to treat the cancer.

Also provided herein are methods and uses involving administration of an effective amount of an anti-TIGIT antagonist antibody (e.g., an anti-TIGIT antagonist antibody as disclosed herein, e.g., tiragolumab), an anti-PD-1 antagonist antibody, and a VEGF antagonist (e.g., an anti-VEGF antibody (e.g., bevacizumab)) to a subject in need thereof, wherein the anti-PD-1 antagonist antibody is pembrolizumab. In some instances, the anti-TIGIT antagonist antibody (e.g., anti-TIGIT antagonist antibody as disclosed herein, e.g., tiragolumab), an anti-PD-1 antagonist antibody, and a VEGF antagonist (e.g., an anti-VEGF antibody (e.g., bevacizumab)) are administered every three weeks (e.g., on Day 1 of each 21-day dosing cycle), wherein the anti-PD-1 antagonist antibody is pembrolizumab.

In particular embodiments, the method involves administration of an effective amount of an anti-TIGIT antagonist antibody (e.g., an anti-TIGIT antagonist antibody as disclosed herein, e.g., tiragolumab), an anti-PD-1 antagonist antibody, and a VEGF antagonist (e.g., an anti-VEGF antibody (e.g., bevacizumab)) to a subject in need thereof, wherein the anti-TIGIT antagonist antibody (e.g., anti-TIGIT antagonist antibody as disclosed herein, e.g., tiragolumab), the anti-PD-1 antagonist antibody, and the VEGF antagonist (e.g., an anti-VEGF antibody (e.g., bevacizumab)) are administered every three weeks (e.g., on Day 1 of each 21-day dosing cycle), wherein the anti-PD-1 antagonist antibody is pembrolizumab. In some instances, the effective amount of the anti-TIGIT antagonist antibody (e.g., an anti-TIGIT antagonist antibody as disclosed herein, e.g., tiragolumab), the anti-PD-1 antagonist antibody, and the VEGF antagonist (e.g., an anti-VEGF antibody (e.g., bevacizumab)) results in an increase in OS of the subject, wherein the anti-PD-1 antagonist antibody is pembrolizumab. In some instances, the effective amount of the anti-TIGIT antagonist antibody (e.g., an anti-TIGIT antagonist antibody as disclosed herein, e.g., tiragolumab), the anti-PD-1 antagonist antibody, and the VEGF antagonist (e.g., an anti-VEGF antibody (e.g., bevacizumab)) results in an increase in PFS of the subject, wherein the anti-PD-1 antagonist antibody is pembrolizumab.

In some instances, the subject receiving the anti-TIGIT antagonist antibody (e.g., an anti-TIGIT antagonist antibody as disclosed herein, e.g., tiragolumab), the anti-PD-1 antagonist antibody, and the VEGF antagonist (e.g., an anti-VEGF antibody (e.g., bevacizumab)) is being treated for a solid tumor or a locally advanced or metastatic cancer, wherein the anti-PD-1 antagonist antibody is pembrolizumab. Additionally or alternatively, the cancer may be a lung cancer (e.g., an early stage lung cancer (e.g., a resectable lung cancer), a SCLC (e.g., an ES-SCLC), a NSCLC (e.g., a squamous NSCLC or a non-squamous NSCLC, a locally advanced unresectable NSCLC, a Stage IIIB NSCLC, a recurrent or metastatic NSCLC (e.g., a locally advanced unresectable or metastatic non-squamous NSCLC (e.g., Stage IV non-squamous NSCLC)), or a Stage IV NSCLC (e.g., wherein the subject has not been previously treated for Stage IV NSCLC))); a cervical cancer (e.g., a Stage IVB, metastatic, recurrent, or persistent cervical cancer, e.g., a metastatic and/or recurrent PD-L1-positive cervical carcinoma); a breast cancer (e.g., a TNBC (e.g., an eTNBC)) or a HER2-positive breast cancer); a head and neck cancer (e.g., SCCHN, e.g., recurrent/metastatic PD-L1-positive SCCHN); a liver cancer (e.g., HCC, e.g., locally advanced or metastatic HCC and/or unresectable HCC); a bladder cancer (e.g., MIBC, locally advanced UC, or mUC); an esophageal cancer; a pancreatic cancer (e.g., PDAC, e.g., metastatic PDAC); a kidney or renal cancer (e.g., a RCC); a melanoma; an ovarian cancer; a gastric cancer (e.g., a gastroesophageal junction cancer); or a CRC (e.g., MSS or MSI-Low CRC).

Also provided herein are methods and uses involving administration of an effective amount of tiragolumab and pembrolizumab to a subject in need thereof. In some instances, tiragolumab is administered every three weeks (e.g., on Day 1 and Day 22 of each 42-day dosing cycle) and pembrolizumab is administered every six weeks (e.g., on Day 1 of each 42-day dosing cycle).

In particular embodiments, the method involves administration of an effective amount of tiragolumab and pembrolizumab to a subject in need thereof, wherein tiragolumab is administered every three weeks (e.g., on Day 1 and Day 22 of each 42-day dosing cycle) and pembrolizumab is administered every six weeks (e.g., on Day 1 of each 42-day dosing cycle). In some instances, the effective amount of tiragolumab and pembrolizumab results in an increase in OS of the subject, wherein the anti-PD-1 antagonist antibody is pembrolizumab. In some instances, the effective amount of tiragolumab and pembrolizumab results in an increase in PFS of the subject, wherein the anti-PD-1 antagonist antibody is pembrolizumab.

In some instances, the subject receiving tiragolumab and pembrolizumab is being treated for a solid tumor or a locally advanced or metastatic cancer, wherein the anti-PD-1 antagonist antibody is pembrolizumab. Additionally or alternatively, the cancer may be a lung cancer (e.g., an early stage lung cancer (e.g., a resectable lung cancer), a SCLC (e.g., an ES-SCLC), a NSCLC (e.g., a squamous NSCLC or a non-squamous NSCLC, a locally advanced unresectable NSCLC, a Stage IIIB NSCLC, a recurrent or metastatic NSCLC (e.g., a locally advanced unresectable or metastatic non-squamous NSCLC (e.g., Stage IV non-squamous NSCLC)), or a Stage IV NSCLC (e.g., wherein the subject has not been previously treated for Stage IV NSCLC))); a cervical cancer (e.g., a Stage IVB, metastatic, recurrent, or persistent cervical cancer, e.g., a metastatic and/or recurrent PD-L1-positive cervical carcinoma); a breast cancer (e.g., a TNBC (e.g., an eTNBC)) or a HER2-positive breast cancer); a head and neck cancer (e.g., SCCHN, e.g., recurrent/metastatic PD-L1-positive SCCHN); a liver cancer (e.g., HCC, e.g., locally advanced or metastatic HCC and/or unresectable HCC); a bladder cancer (e.g., MIBC, locally advanced UC, or mUC); an esophageal cancer; a pancreatic cancer (e.g., PDAC, e.g., metastatic PDAC); a kidney or renal cancer (e.g., a RCC); a melanoma; an ovarian cancer; a gastric cancer (e.g., a gastroesophageal junction cancer); or a CRC (e.g., MSS or MSI-Low CRC).

Also provided herein are methods of treating a subject having a cancer, the methods comprising administering to the subject a dosing regimen comprising one or more dosing cycles of an anti-TIGIT antagonist antibody at a dose of about 500 mg to about 700 mg every three weeks, a PD-1 axis binding antagonist at a dose of about 900 mg to about 1500 mg every three weeks, a platinum-based chemotherapeutic agent every three weeks, and a non-platinum-based chemotherapeutic agent every three weeks. In some instances, the method comprises an induction phase and a maintenance phase. In some instances, the induction phase and maintenance phase each comprise one or more dosing cycles. In some instances, the maintenance phase does not comprise administration of the platinum-based chemotherapeutic agent. In some instances, the maintenance phase does not comprise administration of the non-platinum-based chemotherapeutic agent. In some instances, the maintenance phase comprises one or more dosing cycles of an anti-TIGIT antagonist antibody at a dose of about 700 mg to about 1000 mg every four weeks and a PD-1 axis binding antagonist at a dose of about 1400 mg to 2000 mg every four weeks.

Also provided herein are methods of treating a subject having a cancer, the methods comprising administering to the subject a dosing regimen comprising one or more dosing cycles of an anti-TIGIT antagonist antibody at a dose of about 500 mg to about 700 mg every three weeks and an anti-PD-1 antagonist antibody at a dose of about 100 mg to about 300 mg every three weeks, wherein the anti-PD-1 antagonist antibody is pembrolizumab.

Also provided herein are an anti-TIGIT antagonist antibody and a PD-1 axis binding antagonist for use in a method of treating a subject or population of subjects having a cancer, wherein the method is according to a method provided herein.

Also provided herein is use of an anti-TIGIT antagonist antibody in the manufacture of a medicament for treating a subject or population of subjects having a cancer in combination with a PD-1 axis binding antagonist, wherein the treatment is according to a method provided herein. In some aspects, the PD-1 axis binding antagonist are provided in separate formulations. In other aspects, the anti-TIGIT antagonist antibody and the PD-1 axis binding antagonist are provided in a single formulation. In some aspects, tiragolumab and atezolizumab are combined in an IV bag prior to administration.

Dosing of Agents

Dosing of anti-TIGIT antagonist antibodies, PD-1 axis binding antagonists, VEGF antagonists, and chemotherapeutic agents is described in Section III(K).

Cancer Characterization and Selection

In any of the methods, uses, or compositions for use described herein, the cancer may be solid tumor or a locally advanced or metastatic cancer. In some instances, the cancer is a lung cancer (e.g., an early stage lung cancer (e.g., a resectable lung cancer), a SCLC (e.g., an ES-SCLC), a NSCLC (e.g., a squamous NSCLC or a non-squamous NSCLC, a locally advanced unresectable NSCLC, a Stage IIIB NSCLC, a recurrent or metastatic NSCLC (e.g., a locally advanced unresectable or metastatic non-squamous NSCLC (e.g., Stage IV non-squamous NSCLC)), or a Stage IV NSCLC (e.g., wherein the subject has not been previously treated for Stage IV NSCLC))); a cervical cancer (e.g., a Stage IVB, metastatic, recurrent, or persistent cervical cancer, e.g., a metastatic and/or recurrent PD-L1-positive cervical carcinoma); a breast cancer (e.g., a TNBC (e.g., an early TNBC (eTNBC))) or a HER2-positive breast cancer); a head and neck cancer (e.g., SCCHN, e.g., recurrent/metastatic PD-L1-positive SCCHN); a liver cancer (e.g., HCC, e.g., locally advanced or metastatic HCC and/or unresectable HCC); a bladder cancer (e.g., MIBC, locally advanced UC, or mUC); an esophageal cancer; a pancreatic cancer (e.g., PDAC, e.g., metastatic PDAC); a kidney or renal cancer (e.g., a RCC); a melanoma; an ovarian cancer; a gastric cancer (e.g., a gastroesophageal junction cancer); or a CRC (e.g., MSS or MSI-Low CRC). In some instances in which the subject has a breast cancer, the subject has not received prior systemic therapy for metastatic breast cancer.

In some instances, in any of the methods, uses, or compositions for use described herein, the subject has no epidermal growth factor receptor (EGFR) or anaplastic lymphoma kinase (ALK) genomic tumor aberrations. In some instances, in any of the methods, uses, or compositions for use described herein, the subject does not have a sensitizing EGFR gene mutation or ALK gene rearrangement. In some instances, the subject has an Eastern Cooperative Oncology Group (ECOG) Performance Status (PS) of 0 or 1.

Methods for detecting the mutational status EGFR and ALK are well known in the art, and include, but are not limited to, sequencing DNA from clinical samples (e.g., tumor biopsies or blood samples (e.g., circulating tumor DNA in blood)) using a next-generation sequencing method, such as the targeted gene pulldown and sequencing method described in Frampton et al. (Nature Biotechnology. 31(11): 1023-1033, 2013), which is incorporated by reference herein in its entirety. Such a next-generation sequencing method can be used with any of the methods disclosed herein to detect various mutations (e.g., insertions, deletions, base substitutions, focal gene amplifications, and/or homozygous gene deletions), while enabling the use of small samples (e.g., from small-core needle biopsies, fine-needle aspirations, and/or cell blocks) or fixed samples (e.g., formalin-fixed and paraffin-embedded (FFPE) samples). Other methods for the detection of the mutational status of EGFR and ALK include fluorescence in situ hybridization (FISH) and immunohistochemical (IHC) methods. Exemplary methods for the detection of the mutational status of ALK are disclosed in U.S. Pat. No. 9,651,555, which is herein incorporated by reference in its entirety. In some instances, the VENTANA® anti-ALK(D5F3) IHC assay is used to determine the mutational status of the ALK gene.

In some instances of any of the methods described herein, the mutation is a sensitizing EGFR mutation. Sensitizing EGFR mutations are well known in the art and include those described in U.S. Publication No: US 2018/0235968 and in Juan et al. (Therapeutic Advances in Medical Oncology. 9(3): 201-216, 2017), which are incorporated by reference herein in their entireties. In some instances, the sensitizing EGFR mutation is a mutation in any one of exons 18-21 (e.g., a mutation in exon 18, exon 19, exon 20, and/or exon 21). In some instances, the sensitizing EGFR mutation is a deletion of exon 19 (dell 9). In other instances, sensitizing EGFR mutation is a L858R point mutation in exon 21. In some instances, the sensitizing EGFR mutation is a G719X point mutation in exon 18, wherein “X” is most commonly C, A, or S. In some instances, the sensitizing EGFR mutation is a G719S point mutation in exon 18. In some instances, the sensitizing EGFR mutation is a G719A point mutation in exon 18. In some instances, the sensitizing EGFR mutation is a S720F point mutation in exon 18. In some instances, the sensitizing EGFR mutation is a L861Q point mutation in exon 21. In some instances, the sensitizing EGFR mutation is a L861R point mutation in exon 21. In other instances, the sensitizing EGFR mutation is a T790M point mutation. In some instances, the sensitizing EGFR mutation is an E709X point mutation, where “X” is most commonly K, A, or H. In some instances, the sensitizing EGFR mutation is a S768I point mutation.

In some instances of any of the methods described herein, the mutation is an ALK gene rearrangement. ALK gene rearrangements are well known in the art and include those described in U.S. Pat. No. 9,651,555 and in Du et al. (Thoracic Cancer. 9: 423-430, 2018), which are incorporated herein by reference in their entireties. In some instances, the ALK gene rearrangement results in the creation of an oncogenic ALK tyrosine kinase that activates downstream signaling pathways resulting in increased cell proliferation and survival. In some instances, the ALK gene rearrangement is an ALK rearrangement with a gene selected from the group consisting of EML4, KIF5B, KLC1, TFG, TPR, HIP1, STRN, DCTN1, SQSTM1, NPM1, BCL11A, BIRC6, RANBP2, AT/C, CLTC, TMP4, and MSN resulting in the formation of a fusion oncogene. In some instances, the ALK gene rearrangement is an EML4 rearrangement with ALK resulting in the formation of the fusion oncogene EML4-ALK.

In some instances, in any of the methods, uses, or compositions for use described herein, the subject does not have a pulmonary lymphoepithelioma-like carcinoma subtype of NSCLC. Methods for detecting the subtype of NSCLC are well known in the art, and include, but are not limited to, methods of determination by histopathological criteria, or by molecular features (e.g., a subtype characterized by expression of one or a combination of biomarkers (e.g., particular genes or proteins encoded by said genes)). In some instances, the sample is selected from the group consisting of a tissue sample, a whole blood sample, a serum sample, and a plasma sample. In some instances, the tissue sample is a tumor sample.

In some instances, in any of the methods, uses, or compositions for use described herein, the subject does not have an active Epstein-Barr virus (EBV) infection or a known or suspected chronic active EBV infection. Indicators of active or chronic active EBV infections for use in the methods described herein can include, but are not limited to, EBV IgM, EBV IgG, Epstein-Barr nuclear antigen (EBNA), and Epstein-Barr viral particles detected in a sample from the subject (e.g., a blood or serum sample). Methods for detecting the presence of one or more indicators of active or chronic active EBV infection, including EBV IgM, EBV IgG, Epstein-Barr nuclear antigen (EBNA), and Epstein-Barr viral particles in a sample from a subject are well known in the art, and include, but are not limited to, methods involving serological diagnosis (e.g., the detection of EBV DNA (e.g., by PCR analysis of a blood sample for the detection of EBV viral particles) or EBV antigens or anti-EBV antibodies (e.g., detection of EBNA, EBV IgM, or EBV IgG using heterophilic antibodies). In some instances, the sample is selected from the group consisting of a whole blood sample, a serum sample, and a plasma sample. In some instances, the subject is negative for EBV IgM and/or negative by EBV PCR. In some instances, the subject is negative for EBV IgM and/or negative by EBV PCR and is positive for EBV IgG and/or positive for Epstein-Barr nuclear antigen (EBNA). In other instances, the subject is negative for EBV IgG and/or negative for EBNA.

In some instances, in any of the methods, uses, or compositions for use described herein, the subject has a PD-L1 selected tumor (e.g., a tumor PD-L1 expression with a minimum PD-L1-positive tumor cell fraction or TPS 30% (e.g., 50%) as determined by an IHC with the SP263 or 22C3 antibody or a proportion of tumor area occupied by PD-L1 expressing tumor-infiltrating immune cells (ICs) is greater than or equal to 1% in the tumor sample as determined by an IHC with the SP142 antibody). In some instances, the PD-L1 selected tumor is a tumor that has been determined to have a PD-L1-positive tumor cell fraction or PD-L1 TPS of greater than, or equal to, 30% (e.g., greater than, or equal to, 50%) by an immunohistochemical (IHC) assay. In some instances, the PD-L1 selected tumor is a tumor that has been determined to have a proportion of tumor area occupied by PD-L1-expressing immune cells (ICs) greater than or equal to 1% by an immunohistochemical (IHC) assay. In some instances, the IHC assay uses the anti-PD-L1 antibody SP263, 22C3, SP142, or 28-8. In some instances, the IHC assay uses anti-PD-L1 antibody SP263. In some instances, the IHC assay uses anti-PD-L1 antibody SP142. In some instances, the IHC assay uses anti-PD-L1 antibody 22C3. In some instances, the tumor sample has been determined to have a TPS of greater than, or equal to, 50%. In some instances, the PD-L1-positive tumor cell fraction is greater than, or equal to, 50% (e.g., as determined by positive staining with the anti-PD-L1 antibody SP263 (e.g., using the Ventana assay), as determined by positive staining with the anti-PD-L1 antibody 22C3 (e.g., using the pharmDx assay), or as determined by positive staining with the anti-PD-L1 antibody 28-8). In some embodiments, the PD-L1-positive tumor cell fraction is greater than, or equal to, 30%, as determined by positive staining with the anti-PD-L1 antibody SP142. In some instances, the ICs has been determined to be greater than, or equal to, 1% (e.g., as determined using the Ventana (SP142) PD-L1 IHC assay). In some instances, the ICs has been determined to be greater than, or equal to, 5% (e.g., as determined using the Ventana (SP142) PD-L1 IHC assay). In some instances, the ICs has been determined to be greater than, or equal to, 10% (e.g., as determined using the Ventana (SP142) PD-L1 IHC assay). In some instances, the ICs has been determined to be greater than, or equal to, 1% and less than 50% (e.g., as determined using the Ventana (SP142) PD-L1 IHC assay). In some instances, the ICs has been determined to be greater than, or equal to, 1% and less than 30% (e.g., as determined using the Ventana (SP142) PD-L1 IHC assay).

In some instances, in any of the methods, uses, or compositions for use described herein, a tumor sample obtained from the individual has a detectable protein expression level of PD-L1. In some instances, the detectable protein expression level of PD-L1 has been determined by an IHC assay. In some instances, the IHC assay uses anti-PD-L1 antibody SP142. In some instances, the tumor sample has been determined to have a detectable expression level of PD-L1 in greater than, or equal to, 1% of the tumor cells in the tumor sample. In some instances, the tumor sample has been determined to have a detectable expression level of PD-L1 in greater than, or equal to, 1% and less than 5% of the tumor cells in the tumor sample. In some instances, the tumor sample has been determined to have a detectable expression level of PD-L1 in greater than, or equal to, 5% and less than 50% of the tumor cells in the tumor sample. In some instances, the tumor sample has been determined to have a detectable expression level of PD-L1 in greater than, or equal to, 50% of the tumor cells in the tumor sample. In some instances, the tumor sample has been determined to have a detectable expression level of PD-L1 in tumor-infiltrating immune cells that comprise greater than, or equal to, 1% of the tumor sample. In some instances, the tumor sample has been determined to have a detectable expression level of PD-L1 in tumor-infiltrating immune cells that comprise greater than, or equal to, 1% and less than 5% of the tumor sample. In some instances, the tumor sample has been determined to have a detectable expression level of PD-L1 in tumor-infiltrating immune cells that comprise greater than, or equal to, 5% and less than 10% of the tumor sample. In some instances, the tumor sample has been determined to have a detectable expression level of PD-L1 in tumor-infiltrating immune cells that comprise greater than, or equal to, 10% of the tumor sample.

In some instances, in any of the methods, uses, or compositions for use described herein, a tumor sample obtained from the individual has a detectable nucleic acid expression level of PD-L1. In some instances, the detectable nucleic acid expression level of PD-L1 has been determined by RNA-seq, RT-qPCR, qPCR, multiplex qPCR or RT-qPCR, microarray analysis, SAGE, MassARRAY technique, ISH, or a combination thereof. In some instances, the sample is selected from the group consisting of a tissue sample, a whole blood sample, a serum sample, and a plasma sample. In some instances, the tissue sample is a tumor sample. In some instances, the tumor sample comprises tumor-infiltrating immune cells, tumor cells, stromal cells, and any combinations thereof.

Responses to Treatment

In some embodiments of any of the methods described herein, a subject's response to the therapy can be characterized by one or more measures. In some embodiments, the treatment results in a CR or a PR. In some embodiments, the treatment results in an increase in PFS or DOR.

In some instances, the treatment results in an increase in PFS of the subject, e.g., as compared to treatment with the PD-1 axis binding antagonist without the anti-TIGIT antagonist antibody or as compared to treatment with the anti-TIGIT antagonist antibody without the PD-1 axis binding antagonist. For example, in embodiments in which no chemotherapeutic agent is administered (e.g., only an anti-TIGIT antagonist antibody (e.g., tiragolumab) in combination with a PD-1 axis binding antagonist (e.g., atezolizumab) is administered), the treatment may result in an increase in PFS of the subject, e.g., as compared to treatment with the PD-1 axis binding antagonist without the anti-TIGIT antagonist antibody or as compared to treatment with the anti-TIGIT antagonist antibody without the PD-1 axis binding antagonist. In embodiments in which an anti-TIGIT antagonist antibody (e.g., tiragolumab) and a PD-1 axis binding antagonist (e.g., atezolizumab) are administered in combination with one or more chemotherapeutic agents (e.g., a platinum-based chemotherapeutic agent (e.g., carboplatin or cisplatin) and/or one or more non-platinum-based chemotherapeutic agents (e.g., an alkylating agent (e.g., cyclophosphamide), a taxane (e.g., paclitaxel, e.g., nab-paclitaxel), and/or a topoisomerase II inhibitor (e.g., doxorubicin))), the treatment may result in an increase in PFS of the subject, e.g., as compared to (i) treatment with the PD-1 axis binding antagonist and the one or more chemotherapeutic agents without the anti-TIGIT antagonist antibody; (ii) as compared to treatment with the anti-TIGIT antagonist antibody and the one or more chemotherapeutic agents without the PD-1 axis binding antagonist; and/or (iii) as compared to treatment with the PD-1 axis binding antagonist and the anti-TIGIT antagonist antibody without the one or more chemotherapeutic agents.

In some instances, the treatment extends OS of the subject, e.g., as compared to treatment with the PD-1 axis binding antagonist without the anti-TIGIT antagonist antibody or as compared to treatment with the anti-TIGIT antagonist antibody without the PD-1 axis binding antagonist. For example, in embodiments in which no chemotherapeutic agent is administered (e.g., only an anti-TIGIT antagonist antibody (e.g., tiragolumab) in combination with a PD-1 axis binding antagonist (e.g., atezolizumab) is administered), the treatment may result in an increase in OS of the subject, e.g., as compared to treatment with the PD-1 axis binding antagonist without the anti-TIGIT antagonist antibody or as compared to treatment with the anti-TIGIT antagonist antibody without the PD-1 axis binding antagonist. In embodiments in which an anti-TIGIT antagonist antibody (e.g., tiragolumab) and a PD-1 axis binding antagonist (e.g., atezolizumab) are administered in combination with one or more chemotherapeutic agents (e.g., a platinum-based chemotherapeutic agent (e.g., carboplatin or cisplatin) and/or one or more non-platinum-based chemotherapeutic agents (e.g., an antimetabolite (e.g., pemetrexed, gemcitabine, or capecitabine), a taxane (e.g., paclitaxel, e.g., nab-paclitaxel), and/or a topoisomerase II inhibitor (e.g., etoposide))), the treatment may result in an increase in OS of the subject, e.g., as compared to (i) treatment with the PD-1 axis binding antagonist and the one or more chemotherapeutic agents without the anti-TIGIT antagonist antibody; (ii) as compared to treatment with the anti-TIGIT antagonist antibody and the one or more chemotherapeutic agents without the PD-1 axis binding antagonist; and/or (iii) as compared to treatment with the PD-1 axis binding antagonist and the anti-TIGIT antagonist antibody without the one or more chemotherapeutic agents.

Progression-free survival of the subject can be measured according to RECIST v1.1 criteria, as described in Eisenhauer et al., Eur. J. Cancer. 2009, 45:228-47. In some embodiments, PFS is measured as the period of time from the start of treatment to the first occurrence of disease progression as determined by RECIST v1.1 criteria. In some embodiments, PFS is measured as the time from the start of treatment to the time of death.

In some embodiments, a treatment described herein extends the PFS of the subject by at least about 2.4 months (e.g., by 2.4-120 months, by 2.5-100 months, by 3.0-80 months, by 4.0-60 months, by 5.0-48 months, by 6.0-36 months, by 8.0-24 months, or by 10-12 months, e.g., by at least about 2.4 months, 2.5 months, 2.6 months, 2.7 months, 2.8 months, 2.9 months, 3.0 months, 3.1 months, 3.2 months, 3.3 months, 3.4 months, 3.5 months, 3.6 months, 3.7 months, 3.8 months, 3.9 months, 4.0 months, 4.1 months, 4.2 months, 4.3 months, 4.4 months, 4.5 months, 4.6 months, 4.7 months, 4.8 months, 4.9 months, 5.0 months, 5.1 months, 5.2 months, 5.3 months, 5.4 months, 5.5 months, 5.6 months, 5.7 months, 5.8 months, 5.9 months, 6.0 months, 6.5 months, 7.0 months, 7.5 months, 8.0 months, 8.5 months, 9.0 months, 9.5 months, 10 months, 10.5 months, 11 months, 11.5 months, 12 months, 13 months, 14 months, 15 months, 16 months, 17 months, 18 months, 19 months, 20 months, 21 months, 22 months, 23 months, 24 months, 25 months, 26 months, 27 months, 28 months, 29 months, 30 months, 31 months, 32 months, 33 months, 34 months, 35 months, or 36 months). In some embodiments, the treatment extends the PFS of the subject by at least about 4 months (e.g., by 4-120 months, by 5-100 months, by 6-80 months, by 7-60 months, by 8-48 months, by 9-36 months, or by 10-24 months, e.g., by at least about 4.0 months, 4.1 months, 4.2 months, 4.3 months, 4.4 months, 4.5 months, 4.6 months, 4.7 months, 4.8 months, 4.9 months, 5.0 months, 5.1 months, 5.2 months, 5.3 months, 5.4 months, 5.5 months, 5.6 months, 5.7 months, 5.8 months, 5.9 months, 6.0 months, 6.5 months, 7.0 months, 7.5 months, 8.0 months, 8.5 months, 9.0 months, 9.5 months, 10 months, 10.5 months, 11 months, 11.5 months, 12 months, 13 months, 14 months, 15 months, 16 months, 17 months, 18 months, 19 months, 20 months, 21 months, 22 months, 23 months, 24 months, 25 months, 26 months, 27 months, 28 months, 29 months, 30 months, 31 months, 32 months, 33 months, 34 months, 35 months, or 36 months). In some embodiments, the treatment extends the PFS of the subject by at least about 2 months (e.g., by 2-120 months, by 3-100 months, by 4-80 months, by 6-60 months, by 8-48 months, by 9-36 months, or by 10-24 months, e.g., by at least about 2.0 months, 2.1 months, 2.2 months, 2.3 months, 2.4 months, 2.5 months, 2.6 months, 2.7 months, 2.8 months, 2.9 months, 3.0 months, 3.1 months, 3.2 months, 3.3 months, 3.4 months, 3.5 months, 3.6 months, 3.7 months, 3.8 months, 3.9 months, 4.0 months, 4.1 months, 4.2 months, 4.3 months, 4.4 months, 4.5 months, 4.6 months, 4.7 months, 4.8 months, 4.9 months, 5.0 months, 5.1 months, 5.2 months, 5.3 months, 5.4 months, 5.5 months, 5.6 months, 5.7 months, 5.8 months, 5.9 months, 6.0 months, 6.5 months, 7.0 months, 7.5 months, 8.0 months, 8.5 months, 9.0 months, 9.5 months, 10 months, 10.5 months, 11 months, 11.5 months, 12 months, 13 months, 14 months, 15 months, 16 months, 17 months, 18 months, 19 months, 20 months, 21 months, 22 months, 23 months, 24 months, 25 months, 26 months, 27 months, 28 months, 29 months, 30 months, 31 months, 32 months, 33 months, 34 months, 35 months, or 36 months).

In some embodiments, a treatment described herein extends the DOR of the subject by at least about 2.4 months (e.g., by 2.4-120 months, by 2.5-100 months, by 3.0-80 months, by 4.0-60 months, by 5.0-48 months, by 6.0-36 months, by 8.0-24 months, or by 10-12 months, e.g., by at least about 2.4 months, 2.5 months, 2.6 months, 2.7 months, 2.8 months, 2.9 months, 3.0 months, 3.1 months, 3.2 months, 3.3 months, 3.4 months, 3.5 months, 3.6 months, 3.7 months, 3.8 months, 3.9 months, 4.0 months, 4.1 months, 4.2 months, 4.3 months, 4.4 months, 4.5 months, 4.6 months, 4.7 months, 4.8 months, 4.9 months, 5.0 months, 5.1 months, 5.2 months, 5.3 months, 5.4 months, 5.5 months, 5.6 months, 5.7 months, 5.8 months, 5.9 months, 6.0 months, 6.5 months, 7.0 months, 7.5 months, 8.0 months, 8.5 months, 9.0 months, 9.5 months, 10 months, 10.5 months, 11 months, 11.5 months, 12 months, 13 months, 14 months, 15 months, 16 months, 17 months, 18 months, 19 months, 20 months, 21 months, 22 months, 23 months, 24 months, 25 months, 26 months, 27 months, 28 months, 29 months, 30 months, 31 months, 32 months, 33 months, 34 months, 35 months, or 36 months). In some embodiments, the treatment extends the DOR of the subject by at least about 4 months (e.g., by 4-120 months, by 5-100 months, by 6-80 months, by 7-60 months, by 8-48 months, by 9-36 months, or by 10-24 months, e.g., by at least about 4.0 months, 4.1 months, 4.2 months, 4.3 months, 4.4 months, 4.5 months, 4.6 months, 4.7 months, 4.8 months, 4.9 months, 5.0 months, 5.1 months, 5.2 months, 5.3 months, 5.4 months, 5.5 months, 5.6 months, 5.7 months, 5.8 months, 5.9 months, 6.0 months, 6.5 months, 7.0 months, 7.5 months, 8.0 months, 8.5 months, 9.0 months, 9.5 months, 10 months, 10.5 months, 11 months, 11.5 months, 12 months, 13 months, 14 months, 15 months, 16 months, 17 months, 18 months, 19 months, 20 months, 21 months, 22 months, 23 months, 24 months, 25 months, 26 months, 27 months, 28 months, 29 months, 30 months, 31 months, 32 months, 33 months, 34 months, 35 months, or 36 months). In some embodiments, the treatment extends the DOR of the subject by at least about 2 months (e.g., by 2-120 months, by 3-100 months, by 4-80 months, by 6-60 months, by 8-48 months, by 9-36 months, or by 10-24 months, e.g., by at least about 2.0 months, 2.1 months, 2.2 months, 2.3 months, 2.4 months, 2.5 months, 2.6 months, 2.7 months, 2.8 months, 2.9 months, 3.0 months, 3.1 months, 3.2 months, 3.3 months, 3.4 months, 3.5 months, 3.6 months, 3.7 months, 3.8 months, 3.9 months, 4.0 months, 4.1 months, 4.2 months, 4.3 months, 4.4 months, 4.5 months, 4.6 months, 4.7 months, 4.8 months, 4.9 months, 5.0 months, 5.1 months, 5.2 months, 5.3 months, 5.4 months, 5.5 months, 5.6 months, 5.7 months, 5.8 months, 5.9 months, 6.0 months, 6.5 months, 7.0 months, 7.5 months, 8.0 months, 8.5 months, 9.0 months, 9.5 months, 10 months, 10.5 months, 11 months, 11.5 months, 12 months, 13 months, 14 months, 15 months, 16 months, 17 months, 18 months, 19 months, 20 months, 21 months, 22 months, 23 months, 24 months, 25 months, 26 months, 27 months, 28 months, 29 months, 30 months, 31 months, 32 months, 33 months, 34 months, 35 months, or 36 months).

In some embodiments, OS is measured as the period of time from the start of treatment to death. In some instances, the treatment extends the OS of the subject by at least about 2 months (e.g., by 2-120 months, by 3-110 months, by 4-100 months, by 5-80 months, by 6-60 months, by 7-48 months, by 8-36 months, or by 10-24 months, e.g., by at least about 2 months, 2.1 months, 2.2 months, 2.3 months, 2.4 months, 2.5 months, 2.6 months, 2.7 months, 2.8 months, 2.9 months, 3.0 months, 3.1 months, 3.2 months, 3.3 months, 3.4 months, 3.5 months, 3.6 months, 3.7 months, 3.8 months, 3.9 months, 4.0 months, 4.1 months, 4.2 months, 4.3 months, 4.4 months, 4.5 months, 4.6 months, 4.7 months, 4.8 months, 4.9 months, 5.0 months, 5.1 months, 5.2 months, 5.3 months, 5.4 months, 5.5 months, 5.6 months, 5.7 months, 5.8 months, 5.9 months, 6.0 months, 6.5 months, 7.0 months, 7.5 months, 8.0 months, 8.5 months, 9.0 months, 9.5 months, 10 months, 10.5 months, 11 months, 11.5 months, 12 months, 13 months, 14 months, 15 months, 16 months, 17 months, 18 months, 19 months, 20 months, 21 months, 22 months, 23 months, 24 months, 25 months, 26 months, 27 months, 28 months, 29 months, 30 months, 31 months, 32 months, 33 months, 34 months, 35 months, or 36 months). In some instances, the treatment extends the OS of the subject by at least about 3.3 months (e.g., by 3.3-120 months, by 4-100 months, by 5-80 months, by 6-60 months, by 7-48 months, by 8-36 months, or by 10-24 months, e.g., by at least about 3.3 months, 3.4 months, 3.5 months, 3.6 months, 3.7 months, 3.8 months, 3.9 months, 4.0 months, 4.1 months, 4.2 months, 4.3 months, 4.4 months, 4.5 months, 4.6 months, 4.7 months, 4.8 months, 4.9 months, 5.0 months, 5.1 months, 5.2 months, 5.3 months, 5.4 months, 5.5 months, 5.6 months, 5.7 months, 5.8 months, 5.9 months, 6.0 months, 6.5 months, 7.0 months, 7.5 months, 8.0 months, 8.5 months, 9.0 months, 9.5 months, 10 months, 10.5 months, 11 months, 11.5 months, 12 months, 13 months, 14 months, 15 months, 16 months, 17 months, 18 months, 19 months, 20 months, 21 months, 22 months, 23 months, 24 months, 25 months, 26 months, 27 months, 28 months, 29 months, 30 months, 31 months, 32 months, 33 months, 34 months, 35 months, or 36 months). In some instances, the treatment extends the OS of the subject by at least about 5.3 months (e.g., by 5.3-120, by 6-60 months, by 7-48 months, by 8-36 months, or by 10-24 months, e.g., by at least about 5.3 months, 5.5 months, 6.0 months, 6.5 months, 7.0 months, 7.5 months, 8.0 months, 8.5 months, 9.0 months, 9.5 months, 10 months, 10.5 months, 11 months, 11.5 months, 12 months, 13 months, 14 months, 15 months, 16 months, 17 months, 18 months, 19 months, 20 months, 21 months, 22 months, 23 months, 24 months, 25 months, 26 months, 27 months, 28 months, 29 months, 30 months, 31 months, 32 months, 33 months, 34 months, 35 months, or 36 months).

B. Therapeutic Methods and Uses Relating to Lung Cancer

Lung cancer remains the leading cause of cancer deaths worldwide. In the United States, it is the most common cancer in both men and women and accounts for 12%-14% of all new cancer cases. In 2020, there will be an estimated 228,820 new cases of lung cancer, resulting in 135,720 deaths in the United States (Siegel et al. CA Cancer J Clin. 70:7-30 (2020)).

Non-Small Cell Lung Cancer

Non-small cell lung cancer (NSCLC) is the predominant subtype of lung cancer, accounting for approximately 85% of all cases.

Non-small cell lung cancer (NSCLC) is the predominant subtype of lung cancer, accounting for approximately 80%-85% of all cases (Osmani et al. Semin Cancer Biol. 52 (Pt 1):103-9 (2018)). NSCLC can be divided into two major histologic types: adenocarcinoma and squamous cell carcinoma (Travis et al. 2011). Adenocarcinoma histology accounts for approximately 40%-50% of all NSCLC, while squamous cell histology accounts for approximately 20%-30% of NSCLC (Osmani et al. Semin Cancer Biol. 52 (Pt 1):103-9 (2018)). The remaining cases of NSCLC are represented by large cell carcinoma, neuroendocrine tumors, sarcomatoid carcinoma, and are of poorly differentiated histology.

In its early stages, NSCLC is treated surgically with curative intent. However, 30%-70% of patients undergoing resection develop recurrence and die as a result of disease progression (Siegel et al. Cancer Statistics. CA Cancer J Clin. 70:7-30 (2020)). Therefore, there is a high unmet need for improved medical intervention for early-stage NSCLC.

For advanced disease, the overall five-year survival rate is 2%-4%. Poor prognostic factors for survival in patients with NSCLC include advanced stage of disease at the time of initial diagnosis, poor performance status, and a history of unintentional weight loss. More than half of the patients with NSCLC are diagnosed with distant disease, which directly contributes to poor survival prospects.

Despite improvements in the first-line treatment of patients with advanced NSCLC that have resulted in longer survival times and reduced disease-related symptoms, nearly all patients experience disease progression. Cancer immunotherapies, in particular, offer the possibility of long-term disease control. In the metastatic NSCLC setting, PD-L1/PD-1 blocking antibodies (e.g., atezolizumab, nivolumab, and pembrolizumab) provided clinically meaningful benefit in either unselected or PD-L1-selected advanced NSCLC patients; however, a substantial proportion of patients still remained unresponsive or progressed on anti-PD-L1/PD-1 treatment, and the escape mechanisms to such treatment are poorly understood.

Thus, there is an unmet need in the field for the development of efficacious immunotherapies and methods of dosing the same for the treatment (e.g., first-line treatment) of lung cancer (e.g., NSCLC (e.g., non-squamous NSCLC (e.g., locally advanced unresectable or metastatic non-squamous NSCLC (e.g., Stage IV non-squamous NSCLC)))) that achieve a more favorable benefit-risk profile.

Small Cell Lung Cancer

Small cell lung cancer (SCLC) accounts for approximately 15% of all cases of lung cancer. The majority (approximately 70%) of patients with SCLC are diagnosed with extensive-stage disease (ES-SCLC), which has poor survival prospects (median OS approximately 10-12 months). While chemotherapy alone can palliate symptoms and prolong survival for patients with ES-SCLC, long-term survival is rare. The five-year relative survival rate for people with stage I SCLC is approximately 31%. At stage IV, the five-year relative survival rate declines to approximately 2%.

Thus, there is a need in the field for improved treatments for lung cancer (e.g., SCLC, e.g., ES-SCLC).

i. Methods and Uses for Treating Lung Cancer

Provided herein are methods of treating a population of subjects having a lung cancer, the method comprising administering to the population of subjects a dosing regimen comprising one or more dosing cycles of an effective amount of an anti-TIGIT antagonist antibody, a PD-1 axis binding antagonist, a platinum-based chemotherapeutic agent, and a topoisomerase II inhibitor, wherein the treatment results in a median PFS of the population of subjects of at least about 6 months (e.g., at least about 6 months (e.g., between 6 months and 24 months (e.g., between about 6 months to about 15 months (e.g., 6 months, 7 months, 8 months, 9 months, 10 months, 11 months, 12 months, 13 months, 14 months, or 15 months), e.g., between about 6 months to about 13 months (e.g., 6 months, 6.5 months, 7 months, 7.5 months, 8 months, 8.5 months, 9 months, 9.5 months, 10 months, 10.5 months, 11 months, 11.5 months, 12 months, 12.5 months, 13 months)), e.g., between about 8 months to about 10 months (e.g., 8.1 months, 8.2 months, 8.3 months, 8.4 months, 8.5 months, 8.6 months, 8.7 months, 8.8 months, 8.9 months, 9.0 months, 9.1 months, 9.2 months, 9.3 months, 9.4 months, 9.5 months, 9.6 months, 9.7 months, 9.8 months, 9.9 months, 10.0 months)). In some instances, treatment results in a median PFS of the population of subjects of about 8.2 months to about 9.2 months (e.g., about 8.2, 8.4, 8.6, 8.8, 9.0, or 9.2 months, e.g., 8.2-8.4, 8.4-8.6, 8.6-8.8, 8.8-9.0, or 9.0-9.2 months).

Also provided herein are methods of treating a population of subjects having a lung cancer, the method comprising administering to the population of subjects a dosing regimen comprising one or more dosing cycles of an effective amount of an anti-TIGIT antagonist antibody, a PD-1 axis binding antagonist, a platinum-based chemotherapeutic agent, and a topoisomerase II inhibitor, wherein the treatment results in a median OS of the population of subjects of at least about 12 months (e.g., between about 12 months to about 40 months (e.g., between about 12 to about 30 months (e.g., 12 months, 13 months, 14 months, 15 months, 16 months, 17 months, 18 months, 19 months, 20 months, 21 months, 22 months, 23 months, 24 months, 25 months, 26 months, 27 months, 28 months, 29 months, or 30 months), e.g., between about 12 months to about 20 months (e.g., 12 months, 13 months, 14 months, 15 months, 16 months, 17 months, 18 months, 19 months, or 20 months))). In some instances, the treatment results in a median OS of the population of subjects of about 15.3 months to about 17.6 months (e.g., about 15.5, 16, 16.5, 17, 17.5, or 17.6 months, e.g., 15.3-16 months, 16-17 months, or 17-17.6 months).

ii. Methods and Uses for Treating Small Cell Lung Cancer

Provided herein are methods and uses for treating lung cancer (e.g., small cell lung cancer (SCLC), e.g., extensive stage SCLC (ES-SCLC)) in a subject or population of subjects comprising administering to the subject or population of subjects one or more dosing cycles of an effective amount of an anti-TIGIT antagonist antibody (e.g., an anti-TIGIT antagonist antibody as disclosed herein, e.g., tiragolumab), a PD-1 axis binding antagonist (e.g., an anti-PD-L1 antagonist antibody, such as atezolizumab), a platinum-based chemotherapeutic agent (e.g., carboplatin or cisplatin), and a topoisomerase II inhibitor (e.g., etoposide).

Dosing Regimens, Administration, and Response to Treatment

The therapeutic methods and uses of the invention described herein include, in one aspect, administering to a subject or population of subjects having a lung cancer (e.g., SCLC, e.g., ES-SCLC) an effective amount of an anti-TIGIT antagonist antibody (e.g., an anti-TIGIT antagonist antibody as disclosed herein, e.g., tiragolumab), a PD-1 axis binding antagonist (e.g., an anti-PD-L1 antagonist antibody (e.g., atezolizumab)), a platinum-based chemotherapeutic agent, and a topoisomerase II inhibitor, wherein the treatment extends progression-free survival (PFS) of the subject or population of subjects as compared to treatment with the PD-1 axis binding antagonist, the platinum-based chemotherapeutic agent, and the topoisomerase II inhibitor without the anti-TIGIT antagonist antibody, thereby treating the subject or population of subjects. In some instances, the treatment extends OS of the subject or population of subjects as compared to treatment with the PD-1 axis binding antagonist, the platinum-based chemotherapeutic agent, and the topoisomerase II inhibitor without the anti-TIGIT antagonist antibody.

In some embodiments, the PFS of the individual is measured according to RECIST v1.1 criteria, as described in Eisenhauer et al., Eur. J. Cancer. 2009, 45:228-47. In some embodiments, PFS is measured as the period of time from the start of treatment to the first occurrence of disease progression as determined by RECIST v1.1 criteria. In some embodiments, PFS is measured as the time from the start of treatment to the time of death.

In some embodiments, the treatment extends the PFS of the subject or population of subjects by at least about 2.4 months (e.g., by 2.4-120 months, by 2.5-100 months, by 3.0-80 months, by 4.0-60 months, by 5.0-48 months, by 6.0-36 months, by 8.0-24 months, or by 10-12 months, e.g., by at least about 2.4 months, 2.5 months, 2.6 months, 2.7 months, 2.8 months, 2.9 months, 3.0 months, 3.1 months, 3.2 months, 3.3 months, 3.4 months, 3.5 months, 3.6 months, 3.7 months, 3.8 months, 3.9 months, 4.0 months, 4.1 months, 4.2 months, 4.3 months, 4.4 months, 4.5 months, 4.6 months, 4.7 months, 4.8 months, 4.9 months, 5.0 months, 5.5 months, 6.0 months, 6.5 months, 7.0 months, 7.5 months, 8.0 months, 8.5 months, 9.0 months, 9.5 months, 10 months, 10.5 months, 11 months, 11.5 months, 12 months, 13 months, 14 months, 15 months, 16 months, 17 months, 18 months, 19 months, 20 months, 21 months, 22 months, 23 months, 24 months, 25 months, 26 months, 27 months, 28 months, 29 months, 30 months, 31 months, 32 months, 33 months, 34 months, 35 months, or 36 months) as compared to treatment with the PD-1 axis binding antagonist, the platinum-based chemotherapeutic agent, and the topoisomerase II inhibitor without the anti-TIGIT antagonist antibody. In some embodiments, the treatment extends the PFS of the subject or population of subjects by at least about 4 months (e.g., by 4-120 months, by 5-100 months, by 6-80 months, by 7-60 months, by 8-48 months, by 9-36 months, or by 10-24 months, e.g., by at least about 4.0 months, 4.1 months, 4.2 months, 4.3 months, 4.4 months, 4.5 months, 4.6 months, 4.7 months, 4.8 months, 4.9 months, 5.0 months, 5.5 months, 6.0 months, 6.5 months, 7.0 months, 7.5 months, 8.0 months, 8.5 months, 9.0 months, 9.5 months, 10 months, 10.5 months, 11 months, 11.5 months, 12 months, 13 months, 14 months, 15 months, 16 months, 17 months, 18 months, 19 months, 20 months, 21 months, 22 months, 23 months, 24 months, 25 months, 26 months, 27 months, 28 months, 29 months, 30 months, 31 months, 32 months, 33 months, 34 months, 35 months, or 36 months) as compared to treatment with the PD-1 axis binding antagonist, the platinum-based chemotherapeutic agent, and the topoisomerase II inhibitor without the anti-TIGIT antagonist antibody. In some embodiments, the treatment extends the PFS of the subject or population of subjects by at least about 2 months (e.g., by 2-120 months, by 3-100 months, by 4-80 months, by 6-60 months, by 8-48 months, by 9-36 months, or by 10-24 months, e.g., by at least about 2.0 months, 2.1 months, 2.2 months, 2.3 months, 2.4 months, 2.5 months, 2.6 months, 2.7 months, 2.8 months, 2.9 months, 3.0 months, 3.1 months, 3.2 months, 3.3 months, 3.4 months, 3.5 months, 3.6 months, 3.7 months, 3.8 months, 3.9 months, 4.0 months, 4.1 months, 4.2 months, 4.3 months, 4.4 months, 4.5 months, 4.6 months, 4.7 months, 4.8 months, 4.9 months, 5.0 months, 5.5 months, 6.0 months, 6.5 months, 7.0 months, 7.5 months, 8.0 months, 8.5 months, 9.0 months, 9.5 months, 10 months, 10.5 months, 11 months, 11.5 months, 12 months, 13 months, 14 months, 15 months, 16 months, 17 months, 18 months, 19 months, 20 months, 21 months, 22 months, 23 months, 24 months, 25 months, 26 months, 27 months, 28 months, 29 months, 30 months, 31 months, 32 months, 33 months, 34 months, 35 months, or 36 months) as compared to treatment with the PD-1 axis binding antagonist, the platinum-based chemotherapeutic agent, and the topoisomerase II inhibitor without the anti-TIGIT antagonist antibody. In some aspects, the treatment extends the PFS of the subject or population of subjects by at least about 3 months to about 4 months as compared to treatment with the PD-1 axis binding antagonist, the platinum-based chemotherapeutic agent, and the topoisomerase II inhibitor without the anti-TIGIT antagonist antibody.

In some instances, the methods and uses of treating a subject or population of subjects having a lung cancer (e.g., SCLC, e.g., ES-SCLC) include administering to the subject or population of subjects an effective amount of an anti-TIGIT antagonist antibody (e.g., an anti-TIGIT antagonist antibody as disclosed herein, e.g., tiragolumab), a PD-1 axis binding antagonist (e.g., an anti-PD-L1 antagonist antibody, such as atezolizumab), a platinum-based chemotherapeutic agent (e.g., carboplatin or cisplatin), and a topoisomerase II inhibitor (e.g., etoposide), wherein the treatment extends OS of the subject or population of subjects as compared to treatment with the PD-1 axis binding antagonist, the platinum-based chemotherapeutic agent, and the topoisomerase II inhibitor without the anti-TIGIT antagonist antibody.

In some embodiments, OS is measured as the period of time from the start of treatment to death. In some instances, the treatment extends the OS of the subject or population of subjects by at least about 2 months (e.g., by 2-120 months, by 3-110 months, by 4-100 months, by 5-80 months, by 6-60 months, by 7-48 months, by 8-36 months, or by 10-24 months, e.g., by at least about 2 months, 2.1 months, 2.2 months, 2.3 months, 2.4 months, 2.5 months, 2.6 months, 2.7 months, 2.8 months, 2.9 months, 3.0 months, 3.1 months, 3.2 months, 3.3 months, 3.4 months, 3.5 months, 3.6 months, 3.7 months, 3.8 months, 3.9 months, 4.0 months, 4.1 months, 4.2 months, 4.3 months, 4.4 months, 4.5 months, 4.6 months, 4.7 months, 4.8 months, 4.9 months, 5.0 months, 5.1 months, 5.2 months, 5.3 months, 5.4 months, 5.5 months, 5.6 months, 5.7 months, 5.8 months, 5.9 months, 6.0 months, 6.5 months, 7.0 months, 7.5 months, 8.0 months, 8.5 months, 9.0 months, 9.5 months, 10 months, 10.5 months, 11 months, 11.5 months, 12 months, 13 months, 14 months, 15 months, 16 months, 17 months, 18 months, 19 months, 20 months, 21 months, 22 months, 23 months, 24 months, 25 months, 26 months, 27 months, 28 months, 29 months, 30 months, 31 months, 32 months, 33 months, 34 months, 35 months, or 36 months) as compared to treatment with the PD-1 axis binding antagonist, the platinum-based chemotherapeutic agent, and the topoisomerase II inhibitor without the anti-TIGIT antagonist antibody. In some instances, the treatment extends the OS of the subject or population of subjects by at least about 3.3 months (e.g., by 3.3-120 months, by 4-100 months, by 5-80 months, by 6-60 months, by 7-48 months, by 8-36 months, or by 10-24 months, e.g., by at least about 3.3 months, 3.4 months, 3.5 months, 3.6 months, 3.7 months, 3.8 months, 3.9 months, 4.0 months, 4.1 months, 4.2 months, 4.3 months, 4.4 months, 4.5 months, 4.6 months, 4.7 months, 4.8 months, 4.9 months, 5.0 months, 5.5 months, 6.0 months, 6.5 months, 7.0 months, 7.5 months, 8.0 months, 8.5 months, 9.0 months, 9.5 months, 10 months, 10.5 months, 11 months, 11.5 months, 12 months, 13 months, 14 months, 15 months, 16 months, 17 months, 18 months, 19 months, 20 months, 21 months, 22 months, 23 months, 24 months, 25 months, 26 months, 27 months, 28 months, 29 months, 30 months, 31 months, 32 months, 33 months, 34 months, 35 months, or 36 months) as compared to treatment with the PD-1 axis binding antagonist, the platinum-based chemotherapeutic agent, and the topoisomerase II inhibitor without the anti-TIGIT antagonist antibody. In some instances, the treatment extends the OS of the subject or population of subjects by at least about 5.3 months (e.g., by 5.3-120, by 6-60 months, by 7-48 months, by 8-36 months, or by 10-24 months, e.g., by at least about 5.3 months, 5.5 months, 6.0 months, 6.5 months, 7.0 months, 7.5 months, 8.0 months, 8.5 months, 9.0 months, 9.5 months, 10 months, 10.5 months, 11 months, 11.5 months, 12 months, 13 months, 14 months, 15 months, 16 months, 17 months, 18 months, 19 months, 20 months, 21 months, 22 months, 23 months, 24 months, 25 months, 26 months, 27 months, 28 months, 29 months, 30 months, 31 months, 32 months, 33 months, 34 months, 35 months, or 36 months) as compared to treatment with the PD-1 axis binding antagonist, the platinum-based chemotherapeutic agent, and the topoisomerase II inhibitor without the anti-TIGIT antagonist antibody.

In some instances, the effective amount of the anti-TIGIT antagonist antibody (e.g., an anti-TIGIT antagonist antibody as disclosed herein, e.g., tiragolumab) is a dose (e.g. a fixed dose) of between about 30 mg to about 1200 mg (e.g., between about 30 mg to about 1100 mg, e.g., between about 60 mg to about 1000 mg, e.g., between about 100 mg to about 900 mg, e.g., between about 200 mg to about 800 mg, e.g., between about 300 mg to about 800 mg, e.g., between about 400 mg to about 800 mg, e.g., between about 400 mg to about 750 mg, e.g., between about 450 mg to about 750 mg, e.g., between about 500 mg to about 700 mg, e.g., between about 550 mg to about 650 mg, e.g., 600 mg±10 mg, e.g., 600±6 mg, e.g., 600±5 mg, e.g., 600±3 mg, e.g., 600±1 mg, e.g., 600±0.5 mg, e.g., 600 mg) every three weeks. In some instances, the effective amount of the anti-TIGIT antagonist antibody (e.g., an anti-TIGIT antagonist antibody as disclosed herein, e.g., tiragolumab) is a dose (e.g., a fixed dose) of between about 30 mg to about 600 mg (e.g., between about 50 mg to about 600 mg, e.g., between about 60 mg to about 600 mg, e.g., between about 100 mg to about 600 mg, e.g., between about 200 mg to about 600 mg, e.g., between about 200 mg to about 550 mg, e.g., between about 250 mg to about 500 mg, e.g., between about 300 mg to about 450 mg, e.g., between about 350 mg to about 400 mg, e.g., about 375 mg) every three weeks. In some instances, the effective amount of the anti-TIGIT antagonist antibody (e.g., an anti-TIGIT antagonist antibody as disclosed herein, e.g., tiragolumab) is a dose (e.g., a fixed dose) of about 600 mg every three weeks.

In some instances, the effective amount of the anti-TIGIT antagonist antibody (e.g., an anti-TIGIT antagonist antibody as disclosed herein, e.g., tiragolumab) is a dose (e.g. a fixed dose) of between 30 mg to 1200 mg (e.g., between 30 mg to 1100 mg, e.g., between 60 mg to 1000 mg, e.g., between 100 mg to 900 mg, e.g., between 200 mg to 800 mg, e.g., between 300 mg to 800 mg, e.g., between 400 mg to 800 mg, e.g., between 400 mg to 750 mg, e.g., between 450 mg to 750 mg, e.g., between 500 mg to 700 mg, e.g., between 550 mg to 650 mg, e.g., 600 mg±10 mg, e.g., 600±6 mg, e.g., 600±5 mg, e.g., 600±3 mg, e.g., 600±1 mg, e.g., 600±0.5 mg, e.g., 600 mg) every three weeks. In some instances, the effective amount of the anti-TIGIT antagonist antibody (e.g., an anti-TIGIT antagonist antibody as disclosed herein, e.g., tiragolumab) is a dose (e.g., a fixed dose) of between 30 mg to 600 mg (e.g., between 50 mg to 600 mg, e.g., between 60 mg to 600 mg, e.g., between 100 mg to 600 mg, e.g., between 200 mg to 600 mg, e.g., between 200 mg to 550 mg, e.g., between 250 mg to 500 mg, e.g., between 300 mg to 450 mg, e.g., between 350 mg to 400 mg, e.g., 375 mg) every three weeks. In some instances, the effective amount of the anti-TIGIT antagonist antibody (e.g., an anti-TIGIT antagonist antibody as disclosed herein, e.g., tiragolumab) is a dose (e.g., a fixed dose) of 600 mg every three weeks.

In some instances, effective amount of the anti-TIGIT antagonist antibody (e.g., an anti-TIGIT antagonist antibody as disclosed herein, e.g., tiragolumab) is a dose (e.g., a fixed dose) dose of 600 mg every three weeks. In some instances, the dose (e.g., fixed dose) of the anti-TIGIT antagonist antibody (e.g., an anti-TIGIT antagonist antibody as disclosed herein, e.g., tiragolumab) administered in a combination therapy (e.g., a combination treatment with a PD-1 axis binding antagonist (e.g., an anti-PD-L1 antagonist antibody, e.g., atezolizumab)), topoisomerase II inhibitor (e.g., etoposide), and/or a platinum-based chemotherapeutic agent (e.g., carboplatin or cisplatin) may be reduced as compared to a standard dose of the anti-TIGIT antagonist antibody administered as a monotherapy.

In some instances, the effective amount of the PD-1 axis binding antagonist (e.g., anti-PD-L1 antagonist antibody (e.g., atezolizumab)) is a dose (e.g., a fixed dose) of between about 80 mg to about 2000 mg (e.g., between about 100 mg to about 1600 mg, e.g., between about 200 mg to about 1600 mg, e.g., between about 300 mg to about 1600 mg, e.g., between about 400 mg to about 1600 mg, e.g., between about 500 mg to about 1600 mg, e.g., between about 600 mg to about 1600 mg, e.g., between about 700 mg to about 1600 mg, e.g., between about 800 mg to about 1600 mg, e.g., between about 900 mg to about 1500 mg, e.g., between about 1000 mg to about 1400 mg, e.g., between about 1050 mg to about 1350 mg, e.g., between about 1100 mg to about 1300 mg, e.g., between about 1150 mg to about 1250 mg, e.g., between about 1175 mg to about 1225 mg, e.g., between about 1190 mg to about 1210 mg, e.g., 1200 mg±5 mg, e.g., 1200±2.5 mg, e.g., 1200±1.0 mg, e.g., 1200±0.5 mg, e.g., 1200 mg) every three weeks. In some instances, the effective amount of the PD-1 axis binding antagonist (e.g., anti-PD-L1 antagonist antibody (e.g., atezolizumab)) is a dose (e.g., a fixed dose) of about 840 mg every two weeks. In some instances, the effective amount of the PD-1 axis binding antagonist (e.g., anti-PD-L1 antagonist antibody (e.g., atezolizumab)) is a dose (e.g., a fixed dose) of about 1200 mg every three weeks. In some instances, the effective amount of the PD-1 axis binding antagonist (e.g., anti-PD-L1 antagonist antibody (e.g., atezolizumab)) is a dose (e.g., a fixed dose) of between 80 mg to 2000 mg (e.g., between 100 mg to 1600 mg, e.g., between 200 mg to 1600 mg, e.g., between 300 mg to 1600 mg, e.g., between 400 mg to 1600 mg, e.g., between 500 mg to 1600 mg, e.g., between 600 mg to 1600 mg, e.g., between 700 mg to 1600 mg, e.g., between 800 mg to 1600 mg, e.g., between 900 mg to 1500 mg, e.g., between 1000 mg to 1400 mg, e.g., between 1050 mg to 1350 mg, e.g., between 1100 mg to 1300 mg, e.g., between 1150 mg to 1250 mg, e.g., between 1175 mg to 1225 mg, e.g., between 1190 mg to 1210 mg, e.g., 1200 mg±5 mg, e.g., 1200±2.5 mg, e.g., 1200±1.0 mg, e.g., 1200±0.5 mg, e.g., 1200 mg) every three weeks. In some instances, the effective amount of the PD-1 axis binding antagonist (e.g., anti-PD-L1 antagonist antibody (e.g., atezolizumab)) is a dose (e.g., a fixed dose) of 840 mg every two weeks.

In some instances, the effective amount of the PD-1 axis binding antagonist (e.g., anti-PD-L1 antagonist antibody (e.g., atezolizumab)) is a dose (e.g., a fixed dose) of about 1400 mg to 2000 mg every four weeks. In some instances, the effective amount of the PD-1 axis binding antagonist (e.g., anti-PD-L1 antagonist antibody (e.g., atezolizumab)) is a dose (e.g., a fixed dose) of 1400 mg to 2000 mg every four weeks. In some instances, the effective amount of the PD-1 axis binding antagonist (e.g., anti-PD-L1 antagonist antibody (e.g., atezolizumab)) is a dose (e.g., a fixed dose) of about 1680 mg every four weeks. In some instances, the effective amount of the PD-1 axis binding antagonist (e.g., anti-PD-L1 antagonist antibody (e.g., atezolizumab)) is a dose (e.g., a fixed dose) of 1680 mg every four weeks. In some instances, the dose (e.g., fixed dose) of the PD-1 axis binding antagonist (e.g., anti-PD-L1 antagonist antibody (e.g., atezolizumab)) administered in a combination therapy (e.g., a combination treatment with an anti-TIGIT antagonist antibody, such as an anti-TIGIT antagonist antibody disclosed herein, e.g., tiragolumab), a topoisomerase II inhibitor (e.g., etoposide), and/or a platinum-based chemotherapeutic agent (e.g., carboplatin or cisplatin) may be reduced as compared to a standard dose of the PD-1 axis binding antagonist administered as a monotherapy.

In some instances, the effective amount of the PD-1 axis binding antagonist (e.g., anti-PD-L1 antagonist antibody (e.g., atezolizumab)) is a dose of between about 0.01 mg/kg to about 50 mg/kg of the subject's body weight (e.g., between about 0.01 mg/kg to about 45 mg/kg, e.g., between about 0.1 mg/kg to about 40 mg/kg, e.g., between about 1 mg/kg to about 35 mg/kg, e.g., between about 2.5 mg/kg to about 30 mg/kg, e.g., between about 5 mg/kg to about 25 mg/kg, e.g., between about 10 mg/kg to about 20 mg/kg, e.g., between about 12.5 mg/kg to about 15 mg/kg, e.g., about 15±2 mg/kg, about 15±1 mg/kg, about 15±0.5 mg/kg, about 15±0.2 mg/kg, or about 15±0.1 mg/kg, e.g., about 15 mg/kg) every three weeks. In some instances, the effective amount of the PD-1 axis binding antagonist (e.g., anti-PD-L1 antagonist antibody (e.g., atezolizumab)) is a dose of between about 0.01 mg/kg to about 15 mg/kg of the subject's body weight (e.g., between about 0.1 mg/kg to about 15 mg/kg, e.g., between about 0.5 mg/kg to about 15 mg/kg, e.g., between about 1 mg/kg to about 15 mg/kg, e.g., between about 2.5 mg/kg to about 15 mg/kg, e.g., between about 5 mg/kg to about 15 mg/kg, e.g., between about 7.5 mg/kg to about 15 mg/kg, e.g., between about 10 mg/kg to about 15 mg/kg, e.g., between about 12.5 mg/kg to about 15 mg/kg, e.g., between about 14 mg/kg to about 15 mg/kg, e.g., about 15±1 mg/kg, e.g., about 15±0.5 mg/kg, e.g., about 15±0.2 mg/kg, e.g., about 15±0.1 mg/kg, e.g., about 15 mg/kg) every three weeks. In some instances, the effective amount of PD-1 axis binding antagonist (e.g., anti-PD-L1 antagonist antibody (e.g., atezolizumab)) is a dose of about 15 mg/kg administered every three weeks.

In some instances, the effective amount of the PD-1 axis binding antagonist (e.g., anti-PD-L1 antagonist antibody (e.g., atezolizumab)) is a dose of between 0.01 mg/kg to 50 mg/kg of the subject's body weight (e.g., between 0.01 mg/kg to 45 mg/kg, e.g., between 0.1 mg/kg to 40 mg/kg, e.g., between 1 mg/kg to 35 mg/kg, e.g., between 2.5 mg/kg to 30 mg/kg, e.g., between 5 mg/kg to 25 mg/kg, e.g., between 10 mg/kg to 20 mg/kg, e.g., between 12.5 mg/kg to 15 mg/kg, e.g., 15±2 mg/kg, 15±1 mg/kg, 15±0.5 mg/kg, 15±0.2 mg/kg, or 15±0.1 mg/kg, e.g., 15 mg/kg) every three weeks. In some instances, the effective amount of the PD-1 axis binding antagonist (e.g., anti-PD-L1 antagonist antibody (e.g., atezolizumab)) is a dose of between 0.01 mg/kg to 15 mg/kg of the subject's body weight (e.g., between 0.1 mg/kg to 15 mg/kg, e.g., between 0.5 mg/kg to 15 mg/kg, e.g., between 1 mg/kg to 15 mg/kg, e.g., between 2.5 mg/kg to 15 mg/kg, e.g., between 5 mg/kg to 15 mg/kg, e.g., between 7.5 mg/kg to 15 mg/kg, e.g., between 10 mg/kg to 15 mg/kg, e.g., between 12.5 mg/kg to 15 mg/kg, e.g., between 14 mg/kg to 15 mg/kg, e.g., 15±1 mg/kg, e.g., 15±0.5 mg/kg, e.g., 15±0.2 mg/kg, e.g., 15±0.1 mg/kg, e.g., 15 mg/kg) every three weeks. In some instances, the effective amount of PD-1 axis binding antagonist (e.g., anti-PD-L1 antagonist antibody (e.g., atezolizumab)) is a dose of 15 mg/kg administered every three weeks.

In some instances, the dose of the PD-1 axis binding antagonist (e.g., anti-PD-L1 antagonist antibody (e.g., atezolizumab)) administered in a combination therapy (e.g., a combination treatment with an anti-TIGIT antagonist antibody, such as an anti-TIGIT antagonist antibody disclosed herein, e.g., tiragolumab), a topoisomerase II inhibitor (e.g., etoposide), and/or a platinum-based chemotherapeutic agent (e.g., carboplatin or cisplatin) may be reduced as compared to a standard dose of the PD-1 axis binding antagonist administered as a monotherapy.

In some instances, the effective amount of the platinum-based chemotherapeutic agent (e.g., carboplatin or cisplatin) is a dose sufficient to achieve an AUC from 1-50 mg/ml/min (e.g., 2-25 mg/ml/min, 3-15 mg/ml/min, 4-10 mg/ml/min, or 5 mg/ml/min, e.g., 2 mg/ml/min, 3 mg/ml/min, 4 mg/ml/min, 5 mg/ml/min, 6 mg/ml/min, 7 mg/ml/min, 8 mg/ml/min, 9 mg/ml/min, 10 mg/ml/min, 11 mg/ml/min, 12 mg/ml/min, 13 mg/ml/min, 14 mg/ml/min, 15 mg/ml/min, 20 mg/ml/min, 25 mg/ml/min, 30 mg/ml/min, 35 mg/ml/min, 40 mg/ml/min, 45 mg/ml/min, 50 mg/ml/min). In some instances, the effective amount of the platinum-based chemotherapeutic agent (e.g., carboplatin or cisplatin) is a dose sufficient to achieve AUC=5 mg/ml/min.

AUC can be calculated using the Calvert formula (Calvert et al., J. Clin. Oncol. 1989, 7:1748-56):

Total dose (mg)=(target AUC)×(glomerular filtration rate [GFR]+25)

In some instances, for example, 1200 mg of atezolizumab is equivalent to an average body weight-based dose of 15 m/kg.

In some instances, the effective amount of the platinum-based chemotherapeutic agent (e.g., carboplatin or cisplatin) is 200 mg-1500 mg (e.g., 300 mg-1200 mg, 400 mg-1100 mg, or 500 mg-1000 mg, e.g., 300 mg-400 mg, 400 mg-500 mg, 500 mg-600 mg, 600 mg-700 mg, 700 mg-750 mg, 750 mg-800 mg, 800 mg-900 mg, 900 mg-1000 mg, 1000 mg-1100 mg, or 1100 mg-1200 mg, e.g., about 200 mg, about 300 mg, about 400 mg, about 500 mg, about 600 mg, about 700 mg, about 800 mg, about 900 mg, about 1000 mg, about 1100 mg, about 1200 mg, about 1300 mg, about 1400 mg, or about 1500 mg). In some instances, the effective amount of the platinum-based chemotherapeutic agent (e.g., carboplatin or cisplatin) is about 500 mg-1000 mg (e.g., about 500 mg, about 600 mg, about 700 mg, about 800 mg, about 900 mg, or about 1000 mg).

In some instances, the effective amount of the topoisomerase II inhibitor (e.g., etoposide) is from 10-1000 mg/m² (e.g., from 20-800 mg/m², from 30-700 mg/m², from 40-500 mg/m², from 50-300 mg/m², from 75-200 mg/m², or from 80-150 mg/m², e.g., about 20 mg/m², about 30 mg/m², about 40 mg/m², about 50 mg/m², about 60 mg/m², about 70 mg/m², about 80 mg/m², about 90 mg/m², about 100 mg/m², about 110 mg/m², about 120 mg/m², about 130 mg/m², about 140 mg/m², about 150 mg/m², about 160 mg/m², about 170 mg/m², about 180 mg/m², about 190 mg/m², about 200 mg/m², about 250 mg/m², about 300 mg/m², about 400 mg/m², about 500 mg/m², about 600 mg/m², about 700 mg/m², about 800 mg/m², about 900 mg/m², or about 1000 mg/m²). In some instances, the effective amount of the topoisomerase II inhibitor (e.g., etoposide) is about 100 mg/m².

In some instances, the effective amount of the platinum-based chemotherapeutic agent (e.g., carboplatin or cisplatin) is 200 mg-1500 mg (e.g., 300 mg-1200 mg, 400 mg-1100 mg, or 500 mg-1000 mg, e.g., 300 mg-400 mg, 400 mg-500 mg, 500 mg-600 mg, 600 mg-700 mg, 700 mg-750 mg, 750 mg-800 mg, 800 mg-900 mg, 900 mg-1000 mg, 1000 mg-1100 mg, or 1100 mg-1200 mg, e.g., 200 mg, 300 mg, 400 mg, 500 mg, 600 mg, 700 mg, 800 mg, 900 mg, 1000 mg, 1100 mg, 1200 mg, 1300 mg, 1400 mg, or 1500 mg). In some instances, the effective amount of the platinum-based chemotherapeutic agent (e.g., carboplatin or cisplatin) is 500 mg-1000 mg (e.g., 500 mg, 600 mg, 700 mg, 800 mg, 900 mg, or 1000 mg).

In some instances, the effective amount of the topoisomerase II inhibitor (e.g., etoposide) is from 10-1000 mg/m² (e.g., from 20-800 mg/m², from 30-700 mg/m², from 40-500 mg/m², from 50-300 mg/m², from 75-200 mg/m², or from 80-150 mg/m², e.g., 20 mg/m², 30 mg/m², 40 mg/m², 50 mg/m², 60 mg/m², 70 mg/m², 80 mg/m², 90 mg/m², 100 mg/m², 110 mg/m², 120 mg/m², 130 mg/m², 140 mg/m², 150 mg/m², 160 mg/m², 170 mg/m², 180 mg/m², 190 mg/m², 200 mg/m², 250 mg/m², 300 mg/m², 400 mg/m², 500 mg/m², 600 mg/m², 700 mg/m², 800 mg/m², 900 mg/m², or 1000 mg/m²). In some instances, the effective amount of the topoisomerase II inhibitor (e.g., etoposide) is 100 mg/m².

In any of the methods and uses of the invention, the anti-TIGIT antagonist antibody (e.g., an anti-TIGIT antagonist antibody as disclosed herein, e.g., tiragolumab), the PD-1 axis binding antagonist (e.g., anti-PD-L1 antagonist antibody (e.g., atezolizumab)), the platinum-based chemotherapeutic agent (e.g., carboplatin or cisplatin), and the topoisomerase II inhibitor (e.g., etoposide)) may be administered in one or more dosing cycles (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 or more dosing cycles). In some instances, dosing cycles of the anti-TIGIT antagonist antibody (e.g., an anti-TIGIT antagonist antibody as disclosed herein, e.g., tiragolumab) and the PD-1 axis binding antagonist (e.g., anti-PD-L1 antagonist antibody (e.g., atezolizumab)) (with or without the platinum-based chemotherapeutic agent (e.g., carboplatin or cisplatin) and/or the topoisomerase II inhibitor (e.g., etoposide)) continue until there is a loss of clinical benefit (e.g., confirmed disease progression, drug resistance, death, or unacceptable toxicity). In some instances, the length of each dosing cycle is about 18 to 24 days (e.g., 15 days, 16 days, 17 days, 18 days, 19 days, 20 days, 21 days, 22 days, 23 days, or 24 days). In some instances, the length of each dosing cycle is about 21 days. In other instances, the length of each dosing cycle is about 14 days. In other instances, the length of each dosing cycle is about 28 days.

In some instances, the anti-TIGIT antagonist antibody (e.g., an anti-TIGIT antagonist antibody as disclosed herein, e.g., tiragolumab) is administered on about Day 1 (e.g., Day 1±3 days) of each dosing cycle. For example, the anti-TIGIT antagonist antibody (e.g., an anti-TIGIT antagonist antibody as disclosed herein, e.g., tiragolumab) is administered intravenously at a dose (e.g., a fixed dose) of about 600 mg on Day 1 of each dosing cycle, e.g., each 21-day cycle (i.e., at a dose of about 600 mg every three weeks). In some instances, the anti-TIGIT antagonist antibody (e.g., an anti-TIGIT antagonist antibody as disclosed herein, e.g., tiragolumab) is administered intravenously at a dose (e.g., a fixed dose) of 600 mg on Day 1 of each dosing cycle, e.g., each 21-day cycle (i.e., at a dose of 600 mg every three weeks). Similarly, in some instances, the PD-1 axis binding antagonist (e.g., anti-PD-L1 antagonist antibody (e.g., atezolizumab)) is administered on about Day 1 (e.g., Day 1±3 days) of each dosing cycle. For example, in some instances, the PD-1 axis binding antagonist (e.g., anti-PD-L1 antagonist antibody (e.g., atezolizumab)) is administered intravenously at a dose of about 1200 mg on Day 1 of each 21-day cycle (i.e., at a dose of about 1200 mg every three weeks). In other instances, e.g., the PD-1 axis binding antagonist (e.g., anti-PD-L1 antagonist antibody (e.g., atezolizumab)) is administered intravenously at a dose of about 1200 mg on Day 1 of each 21-day cycle (i.e., at a dose of about 1200 mg every three weeks). In some instances, the PD-1 axis binding antagonist (e.g., anti-PD-L1 antagonist antibody (e.g., atezolizumab)) is administered intravenously at a dose of 1200 mg on Day 1 of each 21-day cycle (i.e., at a dose of 1200 mg every three weeks). In some instances, both the anti-TIGIT antagonist antibody (e.g., an anti-TIGIT antagonist antibody as disclosed herein, e.g., tiragolumab) and the PD-1 axis binding antagonist (e.g., anti-PD-L1 antagonist antibody (e.g., atezolizumab)) are administered on about Day 1 (e.g., Day 1±3 days) of each dosing cycle. In some instances, the platinum-based chemotherapeutic agent is administered on about Day 1 (e.g., Day 1±3 days) of each dosing cycle. In some instances, the topoisomerase II inhibitor is on about Day 1 (e.g., Day 1±3 days) of each dosing cycle. For example, in some instances, the anti-TIGIT antagonist antibody (e.g., an anti-TIGIT antagonist antibody as disclosed herein, e.g., tiragolumab) is administered intravenously at a dose of about 600 mg on Day 1 of each 21-day cycle (i.e., at a dose of about 600 mg every three weeks), and the PD-1 axis binding antagonist (e.g., anti-PD-L1 antagonist antibody (e.g., atezolizumab)) is administered intravenously at a dose of about 1200 mg on Day 1 of each 21-day cycle (i.e., at a dose of about 1200 mg every three weeks), the platinum-based chemotherapeutic agent (e.g., carboplatin or cisplatin) is administered at a dose sufficient to achieve AUC=5 mg/ml/min on Day 1 of each of the four initial dosing cycles, and the topoisomerase II inhibitor (e.g., etoposide) is administered at a dose of 100 mg/m² on each of Days 1, 2, and 3 of each of the four initial dosing cycles. In some instances, the anti-TIGIT antagonist antibody (e.g., an anti-TIGIT antagonist antibody as disclosed herein, e.g., tiragolumab) is administered intravenously at a dose of 600 mg on Day 1 of each 21-day cycle (i.e., at a dose of 600 mg every three weeks), and the PD-1 axis binding antagonist (e.g., anti-PD-L1 antagonist antibody (e.g., atezolizumab)) is administered intravenously at a dose of1200 mg on Day 1 of each 21-day cycle (i.e., at a dose of 1200 mg every three weeks), the platinum-based chemotherapeutic agent (e.g., carboplatin or cisplatin) is administered at a dose sufficient to achieve AUC=5 mg/ml/min on Day 1 of each of the four initial dosing cycles, and the topoisomerase II inhibitor (e.g., etoposide) is administered at a dose of 100 mg/m² on each of Days 1, 2, and 3 of each of the four initial dosing cycles.

In some instances, the anti-TIGIT antagonist antibody, PD-1 axis binding antagonist, platinum-based chemotherapeutic agent, and topoisomerase II inhibitor are administered in each of four initial dosing cycles. In some instances, the anti-TIGIT antagonist antibody is administered at a dose from about 30 mg to about 1200 mg on Day 1 of each of the four initial dosing cycles. In some instances, the anti-TIGIT antagonist antibody is administered at a dose from about 30 mg to about 600 mg on Day 1 of each of the four initial dosing cycles. In some instances, the anti-TIGIT antagonist antibody is administered at a dose of about 600 mg on Day 1 of each of the four initial dosing cycles. In some instances, the PD-1 axis binding antagonist is administered at a dose from about 80 mg to about 1600 mg on Day 1 of each of the four initial dosing cycles (e.g., at a dose of about 1200 mg on Day 1 of each of the four initial dosing cycles). In some instances, the anti-TIGIT antagonist antibody is administered at a dose from 30 mg to 1200 mg on Day 1 of each of the four initial dosing cycles. In some instances, the anti-TIGIT antagonist antibody is administered at a dose from 30 mg to 600 mg on Day 1 of each of the four initial dosing cycles. In some instances, the anti-TIGIT antagonist antibody is administered at a dose of 600 mg on Day 1 of each of the four initial dosing cycles. In some instances, the PD-1 axis binding antagonist is administered at a dose from 80 mg to 1600 mg on Day 1 of each of the four initial dosing cycles (e.g., at a dose of 1200 mg on Day 1 of each of the four initial dosing cycles). In some instances, the platinum-based chemotherapeutic agent is administered at a dose sufficient to achieve AUC=5 mg/ml/min on Day 1 of each of the four initial dosing cycle, and/or the topoisomerase II inhibitor is administered at a dose of 100 mg/m² on each of Days 1, 2, and 3 of each of the four initial dosing cycles.

In some instances, the anti-TIGIT antagonist antibody and the PD-1 axis binding antagonist are further administered in one or more additional cycles following the fourth initial dosing cycle. In some instances, the anti-TIGIT antagonist antibody is administered at a dose from about 30 mg to about 1200 mg on Day 1 of each of the one or more additional dosing cycles (e.g., at a dose from about 30 mg to about 600 mg on Day 1 of each of the one or more additional dosing cycles). In some instances, the anti-TIGIT antagonist antibody is administered at a dose of about 600 mg on Day 1 of each of the one or more additional dosing cycles. In some instances, the PD-1 axis binding antagonist is administered at a dose from about 80 mg to about 2000 mg on Day 1 of each of the one or more additional dosing cycles (e.g., at a dose of about 840 mg, 1200 mg, or 1680 mg on Day 1 of each of the one or more additional dosing cycles). In some instances, the additional dosing cycles include administration of the PD-1 axis binding antagonist (e.g., atezolizumab) at a dose of about 840 mg every two weeks, about 1200 mg every three weeks, or about 1680 mg every four weeks. For example, in some instances, each of the one or more dosing cycles is about 14 days, and the PD-1 axis binding antagonist (e.g., atezolizumab) is administered at a dose of about 840 mg on Day 1 of each of the one or more additional dosing cycles. In some instances, each of the one or more dosing cycles is about 21 days, and the PD-1 axis binding antagonist (e.g., atezolizumab) is administered at a dose of about 1200 mg on Day 1 of each of the one or more additional dosing cycles. In some instances, each of the one or more dosing cycles is about 28 days, and the PD-1 axis binding antagonist (e.g., atezolizumab) is administered at a dose of about 1680 mg on Day 1 of each of the one or more additional dosing cycles.

In some instances, the anti-TIGIT antagonist antibody is administered at a dose from 30 mg to 1200 mg on Day 1 of each of the one or more additional dosing cycles (e.g., at a dose from 30 mg to 600 mg on Day 1 of each of the one or more additional dosing cycles). In some instances, the anti-TIGIT antagonist antibody is administered at a dose of 600 mg on Day 1 of each of the one or more additional dosing cycles. In some instances, the PD-1 axis binding antagonist is administered at a dose from 80 mg to 2000 mg on Day 1 of each of the one or more additional dosing cycles (e.g., at a dose of 840 mg, 1200 mg, or 1680 mg on Day 1 of each of the one or more additional dosing cycles). In some instances, the additional dosing cycles include administration of the PD-1 axis binding antagonist (e.g., atezolizumab) at a dose of 840 mg every two weeks, 1200 mg every three weeks, or 1680 mg every four weeks. For example, in some instances, each of the one or more dosing cycles is 14 days, and the PD-1 axis binding antagonist (e.g., atezolizumab) is administered at a dose of 840 mg on Day 1 of each of the one or more additional dosing cycles. In some instances, each of the one or more dosing cycles is 21 days, and the PD-1 axis binding antagonist (e.g., atezolizumab) is administered at a dose of 1200 mg on Day 1 of each of the one or more additional dosing cycles. In some instances, each of the one or more dosing cycles is 28 days, and the PD-1 axis binding antagonist (e.g., atezolizumab) is administered at a dose of 1680 mg on Day 1 of each of the one or more additional dosing cycles.

In some instances, a subject or population of subjects having lung cancer (e.g., SCLC, e.g., ES-SCLC) is treated by administering to the subject or population of subjects one or more dosing cycles (e.g., 21-day dosing cycles) of an anti-TIGIT antagonist antibody (e.g., an anti-TIGIT antagonist antibody as disclosed herein, e.g., tiragolumab) at a dose from about 30 mg to about 1200 mg (e.g., between about 30 mg to about 1100 mg, e.g., between about 60 mg to about 1000 mg, e.g., between about 100 mg to about 900 mg, e.g., between about 200 mg to about 800 mg, e.g., between about 300 mg to about 800 mg, e.g., between about 400 mg to about 800 mg, e.g., between about 400 mg to about 750 mg, e.g., between about 450 mg to about 750 mg, e.g., between about 500 mg to about 700 mg, e.g., between about 550 mg to about 650 mg, e.g., 600 mg±10 mg, e.g., 600±6 mg, e.g., 600±5 mg, e.g., 600±3 mg, e.g., 600±1 mg, e.g., 600±0.5 mg, e.g., 600 mg) on Day 1 of each dosing cycle, a PD-1 axis binding antagonist (e.g., anti-PD-L1 antagonist antibody (e.g., atezolizumab)) at a dose from about 80 mg to about 2000 mg (e.g., between about 100 mg to about 1600 mg, e.g., between about 200 mg to about 1600 mg, e.g., between about 300 mg to about 1600 mg, e.g., between about 400 mg to about 1600 mg, e.g., between about 500 mg to about 1600 mg, e.g., between about 600 mg to about 1600 mg, e.g., between about 700 mg to about 1600 mg, e.g., between about 800 mg to about 1600 mg, e.g., between about 900 mg to about 1500 mg, e.g., between about 1000 mg to about 1400 mg, e.g., between about 1050 mg to about 1350 mg, e.g., between about 1100 mg to about 1300 mg, e.g., between about 1150 mg to about 1250 mg, e.g., between about 1175 mg to about 1225 mg, e.g., between about 1190 mg to about 1210 mg, e.g., 1200 mg±5 mg, e.g., 1200±2.5 mg, e.g., 1200±1.0 mg, e.g., 1200±0.5 mg, e.g., 1200 mg) on Day 1 of each dosing cycle (e.g., at a dose from about 30 mg to 1200 mg (e.g., between 30 mg to 1100 mg, e.g., between 60 mg to 1000 mg, e.g., between 100 mg to 900 mg, e.g., between 200 mg to 800 mg, e.g., between 300 mg to 800 mg, e.g., between 400 mg to 800 mg, e.g., between 400 mg to 750 mg, e.g., between 450 mg to 750 mg, e.g., between 500 mg to 700 mg, e.g., between 550 mg to 650 mg, e.g., 600 mg±10 mg, e.g., 600±6 mg, e.g., 600±5 mg, e.g., 600±3 mg, e.g., 600±1 mg, e.g., 600±0.5 mg, e.g., 600 mg) on Day 1 of each dosing cycle, a PD-1 axis binding antagonist (e.g., anti-PD-L1 antagonist antibody (e.g., atezolizumab)) at a dose from 80 mg to 2000 mg (e.g., between 100 mg to 1600 mg, e.g., between 200 mg to 1600 mg, e.g., between 300 mg to 1600 mg, e.g., between 400 mg to 1600 mg, e.g., between 500 mg to 1600 mg, e.g., between 600 mg to 1600 mg, e.g., between 700 mg to 1600 mg, e.g., between 800 mg to 1600 mg, e.g., between 900 mg to 1500 mg, e.g., between 1000 mg to 1400 mg, e.g., between 1050 mg to 1350 mg, e.g., between 1100 mg to 1300 mg, e.g., between 1150 mg to 1250 mg, e.g., between 1175 mg to 1225 mg, e.g., between 1190 mg to 1210 mg, e.g., 1200 mg±5 mg, e.g., 1200±2.5 mg, e.g., 1200±1.0 mg, e.g., 1200±0.5 mg, e.g., 1200 mg) on Day 1 of each dosing cycle), a platinum-based chemotherapeutic agent (e.g., carboplatin or cisplatin) at a dose sufficient to achieve AUC from 1-50 mg/ml/min (e.g., 2-25 mg/ml/min, 3-15 mg/ml/min, 4-10 mg/ml/min, or 5 mg/ml/min, e.g., 2 mg/ml/min, 3 mg/ml/min, 4 mg/ml/min, 5 mg/ml/min, 6 mg/ml/min, 7 mg/ml/min, 8 mg/ml/min, 9 mg/ml/min, 10 mg/ml/min, 11 mg/ml/min, 12 mg/ml/min, 13 mg/ml/min, 14 mg/ml/min, 15 mg/ml/min, 20 mg/ml/min, 25 mg/ml/min, 30 mg/ml/min, 35 mg/ml/min, 40 mg/ml/min, 45 mg/ml/min, 50 mg/ml/min, e.g., 5 mg/ml/min) on Day 1 of each dosing cycle, and a topoisomerase II inhibitor (e.g., etoposide) at a dose of 100 mg/m² on each of Days 1, 2, and 3 of each dosing cycle, wherein the treatment extends PFS and/or OS of the subject or population of subjects as compared to treatment with PD-1 axis binding antagonist (e.g., anti-PD-L1 antagonist antibody (e.g., atezolizumab)), platinum-based chemotherapeutic agent (e.g., carboplatin or cisplatin), and topoisomerase II inhibitor (e.g., etoposide) without the anti-TIGIT antagonist antibody (e.g., an anti-TIGIT antagonist antibody as disclosed herein, e.g., tiragolumab).

In some instances, a subject or population of subjects having lung cancer (e.g., SCLC, e.g., ES-SCLC) is treated by administering to the subject or population of subjects one or more dosing cycles (e.g., 21-day dosing cycles) of an anti-TIGIT antagonist antibody (e.g., an anti-TIGIT antagonist antibody as disclosed herein, e.g., tiragolumab) at a dose from 30 mg to 1200 mg (e.g., between 30 mg to 1100 mg, e.g., between 60 mg to 1000 mg, e.g., between 100 mg to 900 mg, e.g., between 200 mg to 800 mg, e.g., between 300 mg to 800 mg, e.g., between 400 mg to 800 mg, e.g., between 400 mg to 750 mg, e.g., between 450 mg to 750 mg, e.g., between 500 mg to 700 mg, e.g., between 550 mg to 650 mg, e.g., 600 mg±10 mg, e.g., 600±6 mg, e.g., 600±5 mg, e.g., 600±3 mg, e.g., 600±1 mg, e.g., 600±0.5 mg, e.g., 600 mg) on Day 1 of each dosing cycle, a PD-1 axis binding antagonist (e.g., anti-PD-L1 antagonist antibody (e.g., atezolizumab)) at a dose from 80 mg to 2000 mg (e.g., between 100 mg to 1600 mg, e.g., between 200 mg to 1600 mg, e.g., between 300 mg to 1600 mg, e.g., between 400 mg to 1600 mg, e.g., between 500 mg to 1600 mg, e.g., between 600 mg to 1600 mg, e.g., between 700 mg to 1600 mg, e.g., between 800 mg to 1600 mg, e.g., between 900 mg to 1500 mg, e.g., between 1000 mg to 1400 mg, e.g., between 1050 mg to 1350 mg, e.g., between 1100 mg to 1300 mg, e.g., between 1150 mg to 1250 mg, e.g., between 1175 mg to 1225 mg, e.g., between 1190 mg to 1210 mg, e.g., 1200 mg±5 mg, e.g., 1200±2.5 mg, e.g., 1200±1.0 mg, e.g., 1200±0.5 mg, e.g., 1200 mg) on Day 1 of each dosing cycle, a platinum-based chemotherapeutic agent (e.g., carboplatin or cisplatin) at a dose sufficient to achieve AUC from 1-50 mg/ml/min (e.g., 2-25 mg/ml/min, 3-15 mg/ml/min, 4-10 mg/ml/min, or 5 mg/ml/min, e.g., 2 mg/ml/min, 3 mg/ml/min, 4 mg/ml/min, 5 mg/ml/min, 6 mg/ml/min, 7 mg/ml/min, 8 mg/ml/min, 9 mg/ml/min, 10 mg/ml/min, 11 mg/ml/min, 12 mg/ml/min, 13 mg/ml/min, 14 mg/ml/min, 15 mg/ml/min, 20 mg/ml/min, 25 mg/ml/min, 30 mg/ml/min, 35 mg/ml/min, 40 mg/ml/min, 45 mg/ml/min, 50 mg/ml/min, e.g., 5 mg/ml/min) on Day 1 of each dosing cycle, and a topoisomerase II inhibitor (e.g., etoposide) at a dose of 100 mg/m² on each of Days 1, 2, and 3 of each dosing cycle, wherein the treatment extends PFS and/or OS of the subject or population of subjects as compared to treatment with PD-1 axis binding antagonist (e.g., anti-PD-L1 antagonist antibody (e.g., atezolizumab)), platinum-based chemotherapeutic agent (e.g., carboplatin or cisplatin), and topoisomerase II inhibitor (e.g., etoposide) without the anti-TIGIT antagonist antibody (e.g., an anti-TIGIT antagonist antibody as disclosed herein, e.g., tiragolumab).

In some instances, the treatment extends the OS of the subject or population of subjects by at least about 3.3 months (e.g., by 3.3-120 months, by 4-100 months, by 5-80 months, by 6-60 months, by 7-48 months, by 8-36 months, or by 10-24 months, e.g., by at least about 3.3 months, 3.4 months, 3.5 months, 3.6 months, 3.7 months, 3.8 months, 3.9 months, 4.0 months, 4.1 months, 4.2 months, 4.3 months, 4.4 months, 4.5 months, 4.6 months, 4.7 months, 4.8 months, 4.9 months, 5.0 months, 5.5 months, 6.0 months, 6.5 months, 7.0 months, 7.5 months, 8.0 months, 8.5 months, 9.0 months, 9.5 months, 10 months, 10.5 months, 11 months, 11.5 months, 12 months, 13 months, 14 months, 15 months, 16 months, 17 months, 18 months, 19 months, 20 months, 21 months, 22 months, 23 months, 24 months, 25 months, 26 months, 27 months, 28 months, 29 months, 30 months, 31 months, 32 months, 33 months, 34 months, 35 months, or 36 months) as compared to treatment with the PD-1 axis binding antagonist, the platinum-based chemotherapeutic agent, and the topoisomerase II inhibitor without the anti-TIGIT antagonist antibody. In some instances, the treatment extends the OS of the subject or population of subjects by at least about 5.3 months (e.g., by 5.3-120, by 6-60 months, by 7-48 months, by 8-36 months, or by 10-24 months, e.g., by at least about 5.3 months, 5.5 months, 6.0 months, 6.5 months, 7.0 months, 7.5 months, 8.0 months, 8.5 months, 9.0 months, 9.5 months, 10 months, 10.5 months, 11 months, 11.5 months, 12 months, 13 months, 14 months, 15 months, 16 months, 17 months, 18 months, 19 months, 20 months, 21 months, 22 months, 23 months, 24 months, 25 months, 26 months, 27 months, 28 months, 29 months, 30 months, 31 months, 32 months, 33 months, 34 months, 35 months, or 36 months) as compared to treatment with the PD-1 axis binding antagonist, the platinum-based 20 chemotherapeutic agent, and the topoisomerase II inhibitor without the anti-TIGIT antagonist antibody.

In some embodiments, the treatment extends the PFS of the subject or population of subjects by at least about 2.4 months (e.g., by 2.4-120 months, by 2.5-100 months, by 3.0-80 months, by 4.0-60 months, by 5.0-48 months, by 6.0-36 months, by 8.0-24 months, or by 10-12 months, e.g., by at least about 2.4 months, 2.5 months, 2.6 months, 2.7 months, 2.8 months, 2.9 months, 3.0 months, 3.1 months, 3.2 months, 3.3 months, 3.4 months, 3.5 months, 3.6 months, 3.7 months, 3.8 months, 3.9 months, 4.0 months, 4.1 months, 4.2 months, 4.3 months, 4.4 months, 4.5 months, 4.6 months, 4.7 months, 4.8 months, 4.9 months, 5.0 months, 5.5 months, 6.0 months, 6.5 months, 7.0 months, 7.5 months, 8.0 months, 8.5 months, 9.0 months, 9.5 months, 10 months, 10.5 months, 11 months, 11.5 months, 12 months, 13 months, 14 months, 15 months, 16 months, 17 months, 18 months, 19 months, 20 months, 21 months, 22 months, 23 months, 24 months, 25 months, 26 months, 27 months, 28 months, 29 months, 30 months, 31 months, 32 months, 33 months, 34 months, 35 months, or 36 months) as compared to treatment with the PD-1 axis binding antagonist, the platinum-based chemotherapeutic agent, and the topoisomerase II inhibitor without the anti-TIGIT antagonist antibody. In some embodiments, the treatment extends the PFS of the subject or population of subjects by at least about 4 months (e.g., by 4-120 months, by 5-100 months, by 6-80 months, by 7-60 months, by 8-48 months, by 9-36 months, or by 10-24 months, e.g., by at least about 4.0 months, 4.1 months, 4.2 months, 4.3 months, 4.4 months, 4.5 months, 4.6 months, 4.7 months, 4.8 months, 4.9 months, 5.0 months, 5.5 months, 6.0 months, 6.5 months, 7.0 months, 7.5 months, 8.0 months, 8.5 months, 9.0 months, 9.5 months, 10 months, 10.5 months, 11 months, 11.5 months, 12 months, 13 months, 14 months, 15 months, 16 months, 17 months, 18 months, 19 months, 20 months, 21 months, 22 months, 23 months, 24 months, 25 months, 26 months, 27 months, 28 months, 29 months, 30 months, 31 months, 32 months, 33 months, 34 months, 35 months, or 36 months) as compared to treatment with the PD-1 axis binding antagonist, the platinum-based chemotherapeutic agent, and the topoisomerase II inhibitor without the anti-TIGIT antagonist antibody.

In some embodiments, the subject or population of subjects receives one or more additional dosing cycles (e.g., 21-day dosing cycles) of the anti-TIGIT antagonist antibody at a dose from about 30 mg to about 1200 mg on Day 1 of each additional dosing cycle and atezolizumab at a dose from about 80 mg to about 2000 mg on Day 1 of each additional dosing cycle, wherein carboplatin and etoposide are omitted from each of the one or more additional dosing cycles.

In some instances, the anti-TIGIT antagonist antibody (e.g., an anti-TIGIT antagonist antibody as disclosed herein, e.g., tiragolumab) is administered to the subject or population of subjects by intravenous infusion over about 60±15 minutes (e.g., about 50 minutes, about 51 minutes, about 52 minutes, about 53 minutes, about 54 minutes, about 55 minutes, about 56 minutes, about 57 minutes, about 58 minutes, about 59 minutes, about 60 minutes, about 61 minutes, about 62 minutes, about 63 minutes, about 64 minutes, about 65 minutes, about 66 minutes, about 67 minutes, about 68 minutes, about 69 minutes, or about 70 minutes). In some instances, the PD-1 axis binding antagonist (e.g., anti-PD-L1 antagonist antibody (e.g., atezolizumab)) is administered to the subject or population of subjects by intravenous infusion over about 60±15 minutes (e.g., about 45 minutes, about 46 minutes, about 47 minutes, about 48 minutes, about 49 minutes, about 50 minutes, about 51 minutes, about 52 minutes, about 53 minutes, about 54 minutes, about 55 minutes, about 56 minutes, about 57 minutes, about 58 minutes, about 59 minutes, about 60 minutes, about 61 minutes, about 62 minutes, about 63 minutes, about 64 minutes, about 65 minutes, about 66 minutes, about 67 minutes, about 68 minutes, about 69 minutes, about 70 minutes, about 71 minutes, about 72 minutes, about 73 minutes, about 74 minutes, or about 75 minutes).

In some instances, the anti-TIGIT antagonist antibody (e.g., an anti-TIGIT antagonist antibody as disclosed herein, e.g., tiragolumab) is administered to the subject or population of subjects before the PD-1 axis binding antagonist (e.g., anti-PD-L1 antagonist antibody (e.g., atezolizumab)). In some instances, for example, following administration of the anti-TIGIT antagonist antibody and before administration of the PD-1 axis binding antagonist, the method includes an intervening first observation period. In some instances, the method further includes a second observation period following administration of the PD-1 axis binding antagonist. In some instances, the method includes both a first observation period following administration of the anti-TIGIT antagonist antibody and second observation period following administration of PD-1 axis binding antagonist. In some instances, the first and second observation periods are each between about 30 minutes to about 60 minutes in length. In instances in which the first and second observation periods are each about 60 minutes in length, the method may include recording the subject's vital signs (e.g., pulse rate, respiratory rate, blood pressure, and temperature) at about 30+10 minutes after administration of the anti-TIGIT antagonist antibody and PD-1 axis binding antagonist during the first and second observation periods, respectively. In instances in which the first and second observation periods are each about 30 minutes in length, the method may include recording the subject's vital signs (e.g., pulse rate, respiratory rate, blood pressure, and temperature) at about 15±10 minutes after administration of the anti-TIGIT antagonist antibody and PD-1 axis binding antagonist during the first and second observation periods, respectively.

In other instances, the PD-1 axis binding antagonist (e.g., anti-PD-L1 antagonist antibody (e.g., atezolizumab)) is administered to the subject or population of subjects before the anti-TIGIT antagonist antibody (e.g., an anti-TIGIT antagonist antibody as disclosed herein, e.g., tiragolumab). In some instances, for example, following administration of the PD-1 axis binding antagonist and before administration of the anti-TIGIT antagonist antibody, the method includes an intervening first observation period. In some instances, the method includes a second observation period following administration of the anti-TIGIT antagonist antibody. In some instances, the method includes both a first observation period following administration of the PD-1 axis binding antagonist and second observation period following administration of the anti-TIGIT antagonist antibody. In some instances, the first and second observation periods are each between about 30 minutes to about 60 minutes in length. In instances in which the first and second observation periods are each about 60 minutes in length, the method may include recording the subject's vital signs (e.g., pulse rate, respiratory rate, blood pressure, and temperature) at about 30±10 minutes after administration of the PD-1 axis binding antagonist and anti-TIGIT antagonist antibody during the first and second observation periods, respectively. In instances in which the first and second observation periods are each about 30 minutes in length, the method may include recording the subject's vital signs (e.g., pulse rate, respiratory rate, blood pressure, and temperature) at about 15±10 minutes after administration of the PD-1 axis binding antagonist and anti-TIGIT antagonist antibody during the first and second observation periods, respectively.

In other instances, the anti-TIGIT antagonist antibody (e.g., an anti-TIGIT antagonist antibody as disclosed herein, e.g., tiragolumab) and the PD-1 axis binding antagonist (e.g., anti-PD-L1 (atezolizumab) antagonist antibody) are administered to the subject or population of subjects simultaneously. In some instances, for example, following administration of the anti-TIGIT antagonist antibody and the PD-1 axis binding antagonist (e.g., anti-PD-L1 antagonist antibody) the method includes an observation period. In some instances, the observation period is between about 30 minutes to about 60 minutes in length. In instances in which the observation period is about 60 minutes in length, the method may include recording the subject's vital signs (e.g., pulse rate, respiratory rate, blood pressure, and temperature) at about 30±10 minutes after administration of the PD-1 axis binding antagonist (e.g., anti-PD-L1 antagonist antibody) and anti-TIGIT antagonist antibody during the observation period. In instances in which the observation period is about 30 minutes in length, the method may include recording the subject's vital signs (e.g., pulse rate, respiratory rate, blood pressure, and temperature) at about 15±10 minutes after administration of the PD-1 axis binding antagonist (e.g., anti-PD-L1 antagonist antibody) and anti-TIGIT antagonist antibody during the observation period.

In any of the methods, uses, or compositions for use described herein, the anti-TIGIT antagonist antibody (e.g., an anti-TIGIT antagonist antibody as disclosed herein, e.g., tiragolumab), PD-1 axis binding antagonist (e.g., anti-PD-L1 antibody (e.g., atezolizumab)), platinum-based chemotherapeutic agent (e.g., carboplatin or cisplatin), and topoisomerase II inhibitor (e.g., etoposide)), or a medicament thereof, may be administered in conjunction with (either separately or together), one or more additional anti-cancer therapeutic agent(s) (e.g., an immunomodulatory agent (e.g., an agent that decreases or inhibits one or more immune co-inhibitory receptors (e.g., one or more immune co-inhibitory receptors selected from TIGIT, PD-L1, PD-1, CTLA-4, LAG3, TIM3, BTLA, and/or VISTA), such as a CTLA-4 antagonist, e.g., an anti-CTLA-4 antagonist antibody (e.g., ipilimumab (YERVOY®)), or an agent that increases or activates one or more immune co-stimulatory receptors (e.g., one or more immune co-stimulatory receptors selected from CD226, OX-40, CD28, CD27, CD137, HVEM, and/or GITR), such as an OX-40 agonist, e.g., an OX-40 agonist antibody), a chemotherapeutic agent, a cytotoxic agent, a growth inhibitory agent, a radiotherapy/radiation therapy, and/or an anti-hormonal agent, such as those recited herein above).

In some instances, the lung cancer is a small cell lung cancer (SCLC), such as extensive stage SCLC (ES-SCLC). In some instances, the subject or population of subjects is treatment-naïve for ES-SCLC (e.g., chemotherapy-naïve for ES-SCLC).

In some instances, the lung cancer is unselected for PD-L1 expression. In other instances, the lung cancer is selected for PD-L1 expression. In some instances, the lung cancer is selected for PD-L1 expression by an immunohistochemical (IHC) assay comprising staining with an anti-PD-L1 antibody, such as SP263, 22C3, SP142, or 28-8. In some instances, the anti-PD-L1 antibody is SP263 and the IHC assay is the Ventana SP263 IHC assay; the anti-PD-L1 antibody is 22C3 and the IHC assay is the pharmDx 22C3 IHC assay; the anti-PD-L1 antibody is SP142 and the IHC assay is the Ventana SP142 IHC assay, or the anti-PD-L1 antibody is 28-8 and the IHC assay is the pharmDx 28-8 IHC assay.

In some instances, in any of the methods, uses, or compositions for use described herein, a tumor sample obtained from the individual has a detectable nucleic acid expression level of PD-L1. In some instances, the detectable nucleic acid expression level of PD-L1 has been determined by RNA-seq, RT-qPCR, qPCR, multiplex qPCR or RT-qPCR, microarray analysis, SAGE, MassARRAY technique, ISH, or a combination thereof. In some instances, the sample is selected from the group consisting of a tissue sample, a whole blood sample, a serum sample, and a plasma sample. In some instances, the tissue sample is a tumor sample. In some instances, the tumor sample comprises tumor-infiltrating immune cells, tumor cells, stromal cells, and any combinations thereof.

In some embodiments, the lung cancer is small cell lung cancer (SCLC). In some embodiments, the SCLC is extensive-stage small cell lung cancer (ES-SCLC), also referred to as stage 4 (IV) SCLC. In some embodiments, the SCLC is histologically or cytologically confirmed ES-SCLC, according to or as defined by the Veterans Administration Lung Study Group (VALG) staging system (see, e.g., Micke et al. Lung Cancer 2002, 37:271-6). In some embodiments, SCLC is classified as ES-SCLC if the individual is inoperable and cannot be classified as having limited or limited stage SCLC (L-SCLC or LS-SCLC). In some embodiments, the ES-SCLC is detectable and/or has spread outside the originally affected lung. In some embodiments, the ESSCLC is detectable and/or has spread further into other (e.g., distant) organs, such as (but not limited to) the liver, adrenal glands, lymph nodes and/or brain. In some embodiments, the ESSCLC is difficult to treat.

In some embodiments, the subject or population of subjects has a poor prognosis. In some embodiments, the subject or population of subjects is a treatment-naïve subject or population of subjects (e.g., a chemotherapy-naïve subject or population of subjects). In some embodiments, a treatment-naïve subject is a subject who has not received prior treatment, e.g., for cancer, for SCLC, or for ES-SCLC. In some embodiments, the treatment naïve subject is a subject who has not received prior treatment for ES-SCLC. In some embodiments, the treatment-naïve subject is chemotherapy naïve, e.g., a subject who has not received prior chemotherapy for the treatment of, e.g., cancer, SCLC, and/or ES-SCLC. In some embodiments, the subject or population of subjects has not received treatment for ES-SCLC. In some embodiments, the subject or population of subjects has not received prior systemic treatment for ES-SCLC. In some embodiments, the subject or population of subjects has received prior chemoradiotherapy for limited stage SCLC (LS-SCLC) with curative intent, and has experienced a treatment-free cycle of at least six months since the last chemotherapy, radiotherapy, or chemoradiotherapy cycle from the diagnosis of ES-SCLC. In some embodiments, the subject or population of subjects has asymptomatic supratentorial or cerebellar central nervous system (CNS) metastases. In some embodiments, the subject or population of subjects does not have metastases to the midbrain, pons, medulla, or spinal cord. In some embodiments, the subject or population of subjects has CNS disease and does not require corticosteroid treatment for CNS disease. In some embodiments, the subject or population of subjects has new asymptomatic metastases and has received radiation therapy and/or surgery for CNS metastases. In some embodiments, the subject or population of subjects has measurable disease, according to/as defined by RECIST v1.1 criteria (see, e.g., Eisenhauer et al., Eur. J. Cancer 2009, 45: 228-247). In some embodiments, the subject or population of subjects has not received prior treatment with a CD137 agonist or an immune checkpoint blockade therapy.

In some instances, the treatment results in a CR or a PR. In some instances, the PFS of the subject or population of subjects is increased as compared to a reference PFS time. In some instances, wherein the reference PFS time is the median PFS time of a population of subjects who have received a treatment with (e.g., anti-PD-L1 antibody (e.g., atezolizumab)), platinum-based chemotherapeutic agent (e.g., carboplatin or cisplatin), and topoisomerase II inhibitor (e.g., etoposide) without the anti-TIGIT antagonist antibody (e.g., tiragolumab).

In some instances, the methods further comprise an additional therapy. The additional therapy may be radiation therapy, surgery (e.g., lumpectomy and a mastectomy), chemotherapy, gene therapy, DNA therapy, viral therapy, RNA therapy, immunotherapy, bone marrow transplantation, nanotherapy, monoclonal antibody therapy, or a combination of the foregoing. The additional therapy may be in the form of adjuvant or neoadjuvant therapy. In some embodiments, the additional therapy is the administration of small molecule enzymatic inhibitor or anti-metastatic agent. In some embodiments, the additional therapy is the administration of side-effect limiting agents (e.g., agents intended to lessen the occurrence and/or severity of side effects of treatment, such as anti-nausea agents, etc.). In some embodiments, the additional therapy is radiation therapy. In some embodiments, the additional therapy is surgery. In some embodiments, the additional therapy is a combination of radiation therapy and surgery. In some embodiments, the additional therapy is gamma irradiation.

Additional therapeutic antibodies contemplated for use herein include, without limitation, alemtuzumab (Campath), bevacizumab (AVASTIN®, Genentech); cetuximab (ERBITUX®, Imclone); panitumumab (VECTIBIX®, Amgen), rituximab (RITUXAN®, Genentech/Biogen Idec), pertuzumab (OMNITARG®, 2C4, Genentech), trastuzumab (HERCEPTIN®, Genentech), tositumomab (Bexxar, Corixia), the antibody drug conjugate gemtuzumab ozogamicin (MYLOTARG®, Wyeth), apolizumab, aselizumab, atlizumab, bapineuzumab, bivatuzumab mertansine, cantuzumab mertansine, cedelizumab, certolizumab pegol, cidfusituzumab, cidtuzumab, daclizumab, eculizumab, efalizumab, epratuzumab, erlizumab, felvizumab, fontolizumab, gemtuzumab ozogamicin, inotuzumab ozogamicin, ipilimumab, labetuzumab, lintuzumab, matuzumab, mepolizumab, motavizumab, motovizumab, natalizumab, nimotuzumab, nolovizumab, numavizumab, ocrelizumab, omalizumab, palivizumab, pascolizumab, pecfusituzumab, pectuzumab, pexelizumab, ralivizumab, ranibizumab, reslivizumab, reslizumab, resyvizumab, rovelizumab, ruplizumab, sibrotuzumab, siplizumab, sontuzumab, tacatuzumab tetraxetan, tadocizumab, talizumab, tefibazumab, tocilizumab, toralizumab, tucotuzumab celmoleukin, tucusituzumab, umavizumab, urtoxazumab, ustekinumab, visilizumab, and the anti-interleukin-12 (ABT-874/J695, Wyeth Research and Abbott Laboratories).

In some embodiments, the additional therapy is therapy targeting PI3K/AKT/mTOR pathway, HSP90 inhibitor, tubulin inhibitor, apoptosis inhibitor, and/or chemopreventative agent. In some embodiments, the additional therapy is CTLA-4 (also known as CD152), e.g., a blocking antibody, ipilimumab (also known as MDX-010, MDX-101, or Yervoy®), tremelimumab (also known as ticilimumab or CP-675,206), an antagonist directed against B7-H3 (also known as CD276), e.g., a blocking antibody, MGA271, an antagonist directed against TGF beta, e.g., metelimumab (also known as CAT-192), fresolimumab (also known as GC1008), or LY2157299, a treatment comprising adoptive transfer of a T cell (e.g., a cytotoxic T cell or CTL) expressing a chimeric antigen receptor (CAR), a treatment comprising adoptive transfer of a T cell comprising a dominant-negative TGF beta receptor, e.g., a dominant-negative TGF beta type II receptor, a treatment comprising a HERCREEM protocol (see, e.g., ClinicalTrials.gov Identifier NCT00889954), an agonist directed against CD137 (also known as TNFRSF9, 4-1BB, or ILA), e.g., an activating antibody, urelumab (also known as BMS-663513), an agonist directed against CD40, e.g., an activating antibody, CP-870893, an agonist directed against OX40 (also known as CD134), e.g., an activating antibody, administered in conjunction with a different anti-OX40 antibody (e.g., AgonOX), an agonist directed against CD27, e.g., an activating antibody, CDX-1127, indoleamine-2,3-dioxygenase (IDO), 1-methyl-D-tryptophan (also known as 1-D-MT), an antibody-drug conjugate (in some embodiments, comprising mertansine or monomethyl auristatin E (MMAE)), an anti-NaPi2b antibody-MMAE conjugate (also known as DNIB0600A or RG7599), trastuzumab emtansine (also known as TDM1, ado-trastuzumab emtansine, or KADCYLA®, Genentech), DMUC5754A, an antibody-drug conjugate targeting the endothelin B receptor (EDNBR), e.g., an antibody directed against EDNBR conjugated with MMAE, an angiogenesis inhibitor, an antibody directed against a VEGF, e.g., VEGF-A, bevacizumab (also known as AVASTIN®, Genentech), an antibody directed against angiopoietin 2 (also known as Ang2), MEDI3617, an antineoplastic agent, an agent targeting CSF-1R (also known as M-CSFR or CD115), anti-CSF-1R (also known as IMCCS4), an interferon, for example interferon alpha or interferon gamma, Roferon-A, GM-CSF (also known as recombinant human granulocyte macrophage colony stimulating factor, rhu GMCSF, sargramostim, or Leukine®), IL-2 (also known as aldesleukin or Proleukin®), IL-12, an antibody targeting CD20 (in some embodiments, the antibody targeting CD20 is obinutuzumab (also known as GA101 or Gazyva®) or rituximab), an antibody targeting GITR (in some embodiments, the antibody targeting GITR is TRX518), in conjunction with a cancer vaccine (in some embodiments, the cancer vaccine is a peptide cancer vaccine, which in some embodiments is a personalized peptide vaccine; in some embodiments the peptide cancer vaccine is a multivalent long peptide, a multi-peptide, a peptide cocktail, a hybrid peptide, or a peptidepulsed dendritic cell vaccine (see, e.g., Yamada et al., Cancer Sci, 104:14-21, 2013)), in conjunction with an adjuvant, a TLR agonist, e.g., Poly-ICLC (also known as Hiltonol®), LPS, MPL, or CpG ODN, tumor necrosis factor (TNF) alpha, IL-1, HMGB1, an IL-10 antagonist, an IL-4 antagonist, an IL-13 antagonist, an HVEM antagonist, an ICOS agonist, e.g., by administration of ICOS-L, or an agonistic antibody directed against ICOS, a treatment targeting CX3CL1, a treatment targeting CXCL10, a treatment targeting CCL5, an LFA-1 or ICAM1 agonist, a Selectin agonist, a targeted therapy, an inhibitor of B-Raf, vemurafenib (also known as Zelboraf®, dabrafenib (also known as Tafinlar®), erlotinib (also known as Tarceva®), an inhibitor of a MEK, such as MEK1 (also known as MAP2K1) or MEK2 (also known as MAP2K2). cobimetinib (also known as GDC-0973 or XL-518), trametinib (also known as Mekinist®), an inhibitor of K-Ras, an inhibitor of c-Met, onartuzumab (also known as MetMAb), an inhibitor of Alk, AF802 (also known as CH5424802 or alectinib), an inhibitor of a phosphatidylinositol 3-kinase (PI3K), BKM120, idelalisib (also known as GS-1101 or CAL 101), perifosine (also known as KRX-0401), an Akt, MK2206, GSK690693, GDC-0941, an inhibitor of mTOR, sirolimus (also known as rapamycin), temsirolimus (also known as CCI-779 or Torisel®), everolimus (also known as RAD001), ridaforolimus (also known as AP-23573, MK-8669, or deforolimus), OSI-027, AZD8055, INK128, a dual PI3K/mTOR inhibitor, XL765, GDC-0980, BEZ235 (also known as NVP-BEZ235), BGT226, GSK2126458, PF-04691502, PF-05212384 (also known as PKI-587). The additional therapy may be one or more of the chemotherapeutic agents described herein.

iii. Methods and Uses for Treating Locally Advanced Unresectable or Metastatic Lung Cancer

Provided herein are methods and uses for treating lung cancer (e.g., non-small cell lung cancer (NSCLC), which includes squamous NSCLC or non-squamous NSCLC, including locally advanced unresectable NSCLC (e.g., Stage IIIB NSCLC), or recurrent or metastatic NSCLC (e.g., Stage IV NSCLC), small cell lung cancer (SCLC), which includes extensive stage SCLC (ES-SCLC), and adenocarcinoma of the lung) in a subject or population of subjects comprising administering to the subject or population of subjects one or more dosing cycles of an effective amount of an anti-TIGIT antagonist antibody (e.g., an anti-TIGIT antagonist antibody as disclosed herein, e.g., tiragolumab), a PD-1 axis binding antagonist (e.g., an anti-PD-L1 antagonist antibody, such as atezolizumab), and a first and second chemotherapeutic agent (e.g., a platinum-based chemotherapeutic agent and a non-platinum-based chemotherapeutic agent).

The present invention includes methods and uses involving administration of an effective amount of an anti-TIGIT antagonist antibody (e.g., an anti-TIGIT antagonist antibody as disclosed herein, e.g., tiragolumab), a PD-1 axis binding antagonist (e.g., an anti-PD-L1 antagonist antibody, such as atezolizumab), and a chemotherapy combination to a subject or population of subjects in need thereof. In some embodiments, the anti-TIGIT antagonist antibody (e.g., anti-TIGIT antagonist antibody as disclosed herein, e.g., tiragolumab) and the PD-1 axis binding antagonist (e.g., an anti-PD-L1 antagonist antibody, e.g., atezolizumab) are administered every three weeks (e.g., on Day 1 of each 21-day dosing cycle). In some aspects, the invention includes methods and uses involving administration of an effective amount of an anti-TIGIT antagonist antibody (e.g., an anti-TIGIT antagonist antibody as disclosed herein, e.g., tiragolumab) and a PD-1 axis binding antagonist (e.g., an anti-PD-L1 antagonist antibody, such as atezolizumab) based on body weight (BW) or body surface area (BSA) of a subject or population of subjects every three weeks (e.g., on Day 1 of each 21-day dosing cycle).

In some aspects, the invention includes methods and uses involving administration of an effective amount of an anti-TIGIT antagonist antibody (e.g., an anti-TIGIT antagonist antibody as disclosed herein, e.g., tiragolumab), a PD-1 axis binding antagonist (e.g., an anti-PD-L1 antagonist antibody, such as atezolizumab), and a chemotherapy combination to a subject or population of subjects in need thereof, wherein the chemotherapy combination includes an effective amount of a platinum-based chemotherapeutic agent and an effective amount of a non-platinum-based chemotherapeutic agent. In some instances, the anti-TIGIT antagonist antibody (e.g., anti-TIGIT antagonist antibody as disclosed herein, e.g., tiragolumab) and the PD-1 axis binding antagonist (e.g., an anti-PD-L1 antagonist antibody, e.g., atezolizumab) are administered every three weeks (e.g., on Day 1 of each 21-day dosing cycle). In some instances, the platinum-based chemotherapeutic agent is carboplatin or cisplatin and the non-platinum-based chemotherapeutic agent is an antimetabolite (e.g., pemetrexed).

In particular embodiments, the method involves administration of an effective amount of an anti-TIGIT antagonist antibody (e.g., an anti-TIGIT antagonist antibody as disclosed herein, e.g., tiragolumab), a PD-1 axis binding antagonist (e.g., an anti-PD-L1 antagonist antibody, e.g., atezolizumab), a platinum-based chemotherapeutic agent (e.g., carboplatin or cisplatin), and an antimetabolite (e.g., pemetrexed) to a subject or population of subjects in need thereof, wherein the anti-TIGIT antagonist antibody (e.g., anti-TIGIT antagonist antibody as disclosed herein, e.g., tiragolumab) and the PD-1 axis binding antagonist (e.g., an anti-PD-L1 antagonist antibody, e.g., atezolizumab) are administered every three weeks (e.g., on Day 1 of each 21-day dosing cycle) and the chemotherapy combination (e.g., the platinum-based chemotherapeutic agent (e.g., carboplatin or cisplatin) and the antimetabolite (e.g., pemetrexed) are administered at the same frequency (e.g., every three weeks, e.g., on Day 1 of each 21-day dosing cycle). In some instances, the dosing continues for four-to-six induction dosing cycles (e.g., four induction dosing cycles, five induction dosing cycles, or six induction dosing cycles). After the induction dosing cycles, maintenance therapy can be administered in one or more subsequent (maintenance) dosing cycles. In certain embodiments, the one or more maintenance dosing cycles does not include the platinum-based chemotherapeutic agent.

In some instances, the present invention includes methods and uses involving administration of an effective amount of an anti-TIGIT antagonist antibody (e.g., an anti-TIGIT antagonist antibody as disclosed herein, e.g., tiragolumab) and a PD-1 axis binding antagonist (e.g., an anti-PD-L1 antagonist antibody, such as atezolizumab) to a subject or population of subjects in need thereof every four weeks (e.g., on Day 1 of each 28-day dosing cycle).

In some instances, administration of the effective amount of the anti-TIGIT antagonist antibody (e.g., an anti-TIGIT antagonist antibody as disclosed herein, e.g., tiragolumab) and the PD-1 axis binding antagonist (e.g., an anti-PD-L1 antagonist antibody, such as atezolizumab) results in a CR or a PR. In some instances, administration of the effective amount of the anti-TIGIT antagonist antibody (e.g., an anti-TIGIT antagonist antibody as disclosed herein, e.g., tiragolumab) and the PD-1 axis binding antagonist (e.g., an anti-PD-L1 antagonist antibody, such as atezolizumab) results in an increase in PFS or DOR. In some instances, administration of the effective amount of the anti-TIGIT antagonist antibody (e.g., an anti-TIGIT antagonist antibody as disclosed herein, e.g., tiragolumab) and the PD-1 axis binding antagonist (e.g., an anti-PD-L1 antagonist antibody, such as atezolizumab) results in an increase in OS. In some instances, administration of the effective amount of the anti-TIGIT antagonist antibody (e.g., an anti-TIGIT antagonist antibody as disclosed herein, e.g., tiragolumab) and the PD-1 axis binding antagonist (e.g., an anti-PD-L1 antagonist antibody, such as atezolizumab) results in an increase in progression-free survival of the subject or population of subjects, e.g., as compared to treatment with the PD-1 axis binding antagonist without the anti-TIGIT antagonist antibody or as compared to treatment with the anti-TIGIT antagonist antibody without the PD-1 axis binding antagonist. In some instances, the effective amount of the anti-TIGIT antagonist antibody (e.g., an anti-TIGIT antagonist antibody as disclosed herein, e.g., tiragolumab) and the PD-1 axis binding antagonist (e.g., an anti-PD-L1 antagonist antibody, such as atezolizumab) extends OS of the subject or population of subjects, e.g., as compared to treatment with the PD-1 axis binding antagonist without the anti-TIGIT antagonist antibody or as compared to treatment with the anti-TIGIT antagonist antibody without the PD-1 axis binding antagonist.

In some instances, administration of the effective amount of an anti-TIGIT antagonist antibody (e.g., an anti-TIGIT antagonist antibody as disclosed herein, e.g., tiragolumab), a PD-1 axis binding antagonist (e.g., an anti-PD-L1 antagonist antibody, such as atezolizumab), and a chemotherapy combination to a subject or population of subjects in need thereof (e.g., a combination of a platinum-based chemotherapeutic agent (e.g., carboplatin or cisplatin) and an antimetabolite (e.g., pemetrexed)) results in an increase in a median PFS of the subject or population of subjects as compared to treatment with pembrolizumab and the chemotherapy combination (e.g., the antimetabolite (e.g., pemetrexed) and the platinum-based chemotherapeutic agent (e.g., carboplatin or cisplatin)). In some instances, the treatment extends the PFS of the subject or population of subjects by at least about 3.5 months or about 4.7 months (e.g., at least about 3.5, 3.6, 3.7, 3.8, 3.9, 4.0, 4.1, 4.2, 4.3, 4.4, 4.5, 4.6, or 4.7 months, e.g., at least about 3.5-3.7 months, 3.7-3.9 months, 3.9-4.1 months, 4.1-4.3 months, 4.3-4.5 months, or 4.5-4.7 months).

In some embodiments, the administration of the effective amount of an anti-TIGIT antagonist antibody (e.g., an anti-TIGIT antagonist antibody as disclosed herein, e.g., tiragolumab), a PD-1 axis binding antagonist (e.g., an anti-PD-L1 antagonist antibody, such as atezolizumab), and a chemotherapy combination (e.g., a combination of a platinum-based chemotherapeutic agent (e.g., carboplatin or cisplatin) and an antimetabolite (e.g., pembrolizumab)) to a subject or population of subjects having a lung cancer (e.g., an NSCLC) according to any of the methods described herein results in a median PFS of greater than 8.8 months (e.g., at least 8.9 months, at least 9.0 months, at least 9.2 months, at least 9.5 months, at least 10 months, at least 11 months, at least 12 months, at least 13 months, at least 14 months, at least 15 months, at least 16 months, at least 17 months, at least 18 months, at least 20 months, at least 24 months, at least 30 months, at least 36 months, at least 42 months, at least 48 months, at least 54 months, or more, e.g., about 8.9 months, about 9.0 months, about 9.2 months, about 9.5 months, about 10 months, about 11 months, about 12 months, about 13 months, about 14 months, about 15 months, about 16 months, about 17 months, about 18 months, about 20 months, about 24 months, about 30 months, about 36 months, about 42 months, about 48 months, about 54 months, or more). In some instances, the treatment results in a median PFS of the population of subjects of at least about 8 months (e.g., between 8 months and 36 months, e.g., between 8 months and 24 months (e.g., 8 months, 9 months, 10 months, 12 months, 13 months, 14 months, 15 months, 16 months, 17 months, 18 months, 19 months, 20 months, 21 months, 22 months, 23 months, or 24 months). In some instances, the treatment results in a median PFS of the population of subjects of about 12.5 months to about 14.7 months (e.g., 12.5, 12.7, 12.9, 13.1, 13.3, 13.5, 13.7, 13.9, 14.1, 14.3, 14.5, or 14.7 months, e.g., about 12.5-13 months, 13-13.5 months, 13.5-14 months, or 14-14.7 months. In some embodiments, the administration of the effective amount of an anti-TIGIT antagonist antibody (e.g., an anti-TIGIT antagonist antibody as disclosed herein, e.g., tiragolumab), a PD-1 axis binding antagonist (e.g., an anti-PD-L1 antagonist antibody, such as atezolizumab), and a chemotherapy combination (e.g., a combination of a platinum-based chemotherapeutic agent (e.g., carboplatin or cisplatin) and an antimetabolite (e.g., pembrolizumab)) to a subject or population of subjects having a lung cancer (e.g., an NSCLC) according to any of the methods described herein results in a median PFS of at least 10 months. In some embodiments, the administration of the effective amount of an anti-TIGIT antagonist antibody (e.g., an anti-TIGIT antagonist antibody as disclosed herein, e.g., tiragolumab), a PD-1 axis binding antagonist (e.g., an anti-PD-L1 antagonist antibody, such as atezolizumab), and a chemotherapy combination (e.g., a combination of a platinum-based chemotherapeutic agent (e.g., carboplatin or cisplatin) and an antimetabolite (e.g., pembrolizumab)) to a subject or population of subjects having a lung cancer (e.g., an NSCLC) according to any of the methods described herein results in a median PFS of at least 12 months.

In some instances, administration of the effective amount of an anti-TIGIT antagonist antibody (e.g., an anti-TIGIT antagonist antibody as disclosed herein, e.g., tiragolumab), a PD-1 axis binding antagonist (e.g., an anti-PD-L1 antagonist antibody, such as atezolizumab), and a chemotherapy combination to a subject or population of subjects having a lung cancer (e.g., a combination of a platinum-based chemotherapeutic agent (e.g., carboplatin or cisplatin) and an antimetabolite (e.g., pemetrexed)) results in an increase in a median OS (OS) of the subject or population of subjects as compared to treatment with pembrolizumab and the chemotherapy combination (e.g., the antimetabolite (e.g., pemetrexed) and the platinum-based chemotherapeutic agent (e.g., carboplatin or cisplatin)). In some instances, the treatment extends the OS of the subject or population of subjects by at least about 4 months (e.g., between 4 and 12 months (e.g., 4 months, 5 months, 6 months, 7 months, 8 months, 9 months, 10 months, 11 months, or 12 months)). In some instances, the treatment extends the OS of the subject or population of subjects by at least about 5.5 months to about 8.0 months (e.g., 5.5, 6.0, 6.5, 7.0, 7.5, or 8.0 months, e.g., 5.5-6.5, 6.5-7.5, or 7.5-8.0 months).

In some embodiments, the administration of the effective amount of an anti-TIGIT antagonist antibody (e.g., an anti-TIGIT antagonist antibody as disclosed herein, e.g., tiragolumab), a PD-1 axis binding antagonist (e.g., an anti-PD-L1 antagonist antibody, such as atezolizumab), and a chemotherapy combination (e.g., a combination of a platinum-based chemotherapeutic agent (e.g., carboplatin or cisplatin) and an antimetabolite (e.g., pembrolizumab)) to a subject or population of subjects having a lung cancer (e.g., an NSCLC) according to any of the methods described herein results in a median OS of greater than 22 months (e.g., at least 23, at least 24, at least 25, at least 26, at least 27, at least 28, at least 29, at least 30, at least 31, at least 32, at least 33, at least 34, at least 35, at least 36, at least 42, at least 48, at least 54, at least 60, at least 70, at least 80, at least 90, at least 100, at least 110, at least 120, at least 130, at least 140, at least 150, at least 160, at least 170, at least 180, at least 190, or at least 200 months, e.g., about 23, about 24, about 25, about 26, about 27, about 28, about 29, about 30, about 31, about 32, about, 33, about 34, about 35, about 36, about 40, about 42, about 48, about 54, about 60, about 70, about 80, about 90, about 100, about 110, about 120, about 130, about 140, about 150, about 160, about 170, about 180, about 190, about 200 months). In some embodiments, the treatment results in a median OS of the population of subjects of at least about 24 months (e.g., between 24 months and 42 months (e.g., between 24 months and 36 months (e.g., 24 months, 25 months, 26 months, 27 months, 28 months, 29 months, 30 months, 31 months, 32 months, 33 months, 34 months, 35 months, or 36 months))). In some embodiments, the treatment results in a median OS of the population of subjects of about 27.5 months to about 32.0 months (e.g., 27.5, 28.0, 28.5, 29.0, 29.5, 30.0, 30.5, 31.0, 31.5, or 32.0 months (e.g., 27.5-28.5, 28.5-29.5, 29.5-30.5, 30.5-31.5, or 31.5-32 months). In some embodiments, the administration of the effective amount of an anti-TIGIT antagonist antibody (e.g., an anti-TIGIT antagonist antibody as disclosed herein, e.g., tiragolumab), a PD-1 axis binding antagonist (e.g., an anti-PD-L1 antagonist antibody, such as atezolizumab), and a chemotherapy combination to a subject or population of subjects in need thereof (e.g., a combination of a platinum-based chemotherapeutic agent (e.g., carboplatin or cisplatin) and an antimetabolite (e.g., pembrolizumab)) to a subject or population of subjects having a lung cancer (e.g., an NSCLC) according to any of the methods described herein results in a median OS of at least 24 months. In some embodiments, the administration of the effective amount of an anti-TIGIT antagonist antibody (e.g., an anti-TIGIT antagonist antibody as disclosed herein, e.g., tiragolumab), a PD-1 axis binding antagonist (e.g., an anti-PD-L1 antagonist antibody, such as atezolizumab), and a chemotherapy combination (e.g., a combination of a platinum-based chemotherapeutic agent (e.g., carboplatin or cisplatin) and an antimetabolite (e.g., pembrolizumab)) to a subject or population of subjects having a lung cancer (e.g., an NSCLC) according to any of the methods described herein results in a median OS of at least 36 months.

In some instances, administration of the effective amount of an anti-TIGIT antagonist antibody (e.g., an anti-TIGIT antagonist antibody as disclosed herein, e.g., tiragolumab), a PD-1 axis binding antagonist (e.g., an anti-PD-L1 antagonist antibody, such as atezolizumab), and a chemotherapy combination to a subject or population of subjects in need thereof (e.g., a combination of a platinum-based chemotherapeutic agent (e.g., carboplatin or cisplatin) and an antimetabolite (e.g., pemetrexed)) results in an increase in an overall response rate (ORR) of the subject or population of subjects as compared to treatment with pembrolizumab and the chemotherapy combination (e.g., the antimetabolite (e.g., pemetrexed) and the platinum-based chemotherapeutic agent (e.g., carboplatin or cisplatin)), e.g., an increase in ORR of at least 10%, (e.g., at least 15%, at least 20%, at least 25%, at least 30%, at least 35%, or at least 40%) as compared to treatment with pembrolizumab and the chemotherapy combination (e.g., the antimetabolite (e.g., pemetrexed) and the platinum-based chemotherapeutic agent (e.g., carboplatin or cisplatin)).

In some embodiments, the administration of the effective amount of an anti-TIGIT antagonist antibody (e.g., an anti-TIGIT antagonist antibody as disclosed herein, e.g., tiragolumab), a PD-1 axis binding antagonist (e.g., an anti-PD-L1 antagonist antibody, such as atezolizumab), and a chemotherapy combination (e.g., a combination of a platinum-based chemotherapeutic agent (e.g., carboplatin or cisplatin) and an antimetabolite (e.g., pembrolizumab)) to a subject or population of subjects having a lung cancer (e.g., an NSCLC) according to any of the methods described herein results in an ORR of greater than 47.5% (e.g., at least 48%, at least 49%, at least 50%, at least 55%, at least 60%, at least 65%, at least 70%, at least 75%, at least 80%, at least 85%, at least 90%, at least 95%, or 100%, e.g., about 48%, about 49%, about 50%, about 55%, about 60%, about 65%, about 70%, about 75%, about 80%, about 85%, about 90%, about 95%, or about 100%). In some embodiments, the administration of the effective amount of an anti-TIGIT antagonist antibody (e.g., an anti-TIGIT antagonist antibody as disclosed herein, e.g., tiragolumab), a PD-1 axis binding antagonist (e.g., an anti-PD-L1 antagonist antibody, such as atezolizumab), and a chemotherapy combination (e.g., a combination of a platinum-based chemotherapeutic agent (e.g., carboplatin or cisplatin) and an antimetabolite (e.g., pembrolizumab)) to a subject or population of subjects having a lung cancer (e.g., an NSCLC) according to any of the methods described herein results in an ORR of at least 50%. In some embodiments, the administration of the effective amount of an anti-TIGIT antagonist antibody (e.g., an anti-TIGIT antagonist antibody as disclosed herein, e.g., tiragolumab), a PD-1 axis binding antagonist (e.g., an anti-PD-L1 antagonist antibody, such as atezolizumab), and a chemotherapy combination (e.g., a combination of a platinum-based chemotherapeutic agent (e.g., carboplatin or cisplatin) and an antimetabolite (e.g., pembrolizumab)) to a subject or population of subjects having a lung cancer (e.g., an NSCLC) according to any of the methods described herein results in an ORR of at least 60%. In some embodiments, the administration of the effective amount of an anti-TIGIT antagonist antibody (e.g., an anti-TIGIT antagonist antibody as disclosed herein, e.g., tiragolumab), a PD-1 axis binding antagonist (e.g., an anti-PD-L1 antagonist antibody, such as atezolizumab), and a chemotherapy combination (e.g., a combination of a platinum-based chemotherapeutic agent (e.g., carboplatin or cisplatin) and an antimetabolite (e.g., pembrolizumab)) to a subject or population of subjects having a lung cancer (e.g., an NSCLC) according to any of the methods described herein results in an ORR of at least 70%.

The present invention includes methods and uses involving administration of an effective amount of an anti-TIGIT antagonist antibody (e.g., an anti-TIGIT antagonist antibody as disclosed herein, e.g., tiragolumab) to a subject or population of subjects in need thereof every four weeks (e.g., on Day 1 of each 28-day dosing cycle). In some instances, administration of the effective amount of the anti-TIGIT antagonist antibody (e.g., an anti-TIGIT antagonist antibody as disclosed herein, e.g., tiragolumab) results in a CR or a PR. In some instances, administration of the effective amount of the anti-TIGIT antagonist antibody (e.g., an anti-TIGIT antagonist antibody as disclosed herein, e.g., tiragolumab) results in an increase in progression-free survival of the subject or population of subjects compared to a reference. In some instances, administration of the effective amount of the anti-TIGIT antagonist antibody (e.g., an anti-TIGIT antagonist antibody as disclosed herein, e.g., tiragolumab) results in an increase in DOR. In some instances, administration of the effective amount of the anti-TIGIT antagonist antibody (e.g., an anti-TIGIT antagonist antibody as disclosed herein, e.g., tiragolumab) extends OS of the subject or population of subjects.

The present invention includes methods and uses involving administration of an effective amount of an anti-TIGIT antagonist antibody (e.g., an anti-TIGIT antagonist antibody as disclosed herein, e.g., tiragolumab) and a PD-1 axis binding antagonist (e.g., an anti-PD-L1 antagonist antibody, such as atezolizumab) to a subject or population of subjects in need thereof every two weeks (e.g., on Days 1 and 15 of each 28-day dosing cycle). In some instances, administration of the effective amount of the anti-TIGIT antagonist antibody (e.g., an anti-TIGIT antagonist antibody as disclosed herein, e.g., tiragolumab) and the PD-1 axis binding antagonist (e.g., an anti-PD-L1 antagonist antibody, such as atezolizumab) results in a CR or a PR. In some instances, administration of the effective amount of the anti-TIGIT antagonist antibody (e.g., an anti-TIGIT antagonist antibody as disclosed herein, e.g., tiragolumab) and the PD-1 axis binding antagonist (e.g., an anti-PD-L1 antagonist antibody, such as atezolizumab) results in an increase in progression-free survival of the subject or population of subjects, e.g., as compared to treatment with the PD-1 axis binding antagonist without the anti-TIGIT antagonist antibody or as compared to treatment with the anti-TIGIT antagonist antibody without the PD-1 axis binding antagonist. In some instances, administration of the effective amount of the anti-TIGIT antagonist antibody (e.g., an anti-TIGIT antagonist antibody as disclosed herein, e.g., tiragolumab) and the PD-1 axis binding antagonist (e.g., an anti-PD-L1 antagonist antibody, such as atezolizumab) extends OS of the subject or population of subjects, e.g., as compared to treatment with the PD-1 axis binding antagonist without the anti-TIGIT antagonist antibody or as compared to treatment with the anti-TIGIT antagonist antibody without the PD-1 axis binding antagonist.

In certain instances, the present invention includes methods and uses involving administration of an effective amount of an anti-TIGIT antagonist antibody (e.g., an anti-TIGIT antagonist antibody as disclosed herein, e.g., tiragolumab) to a subject or population of subjects in need thereof every two weeks (e.g., on Days 1 and 15 of each 28-day dosing cycle). In some instances, administration of the effective amount of the anti-TIGIT antagonist antibody (e.g., an anti-TIGIT antagonist antibody as disclosed herein, e.g., tiragolumab) results in a CR or a PR. In some instances, administration of the effective amount of the anti-TIGIT antagonist antibody (e.g., an anti-TIGIT antagonist antibody as disclosed herein, e.g., tiragolumab) results in an increase in progression-free survival of the subject or population of subjects compared to a reference. In some instances, administration of the effective amount of the anti-TIGIT antagonist antibody (e.g., an anti-TIGIT antagonist antibody as disclosed herein, e.g., tiragolumab) extends OS of the subject or population of subjects.

In some instances, the subject or population of subjects has not received prior systemic therapy (e.g., e.g., prior systemic therapy with curative intent, e.g., chemotherapy) within the month prior to the administration with the PD-1 axis binding antagonist and the anti-TIGIT antagonist antibody (e.g., within the two months prior, three months prior, four months prior, six months prior, one year prior, two years prior, three years prior, four years prior, five years prior, or ten years prior to the administration with the PD-1 axis binding antagonist and the anti-TIGIT antagonist antibody). In some instances, the subject or population of subjects is chemotherapy naïve.

In some embodiments, the PD-1 axis binding antagonist and the anti-TIGIT antagonist antibody are administered in conjunction with a chemotherapy. For example, a once-every-two-weeks (Q2W), once-every-three-weeks (Q3W), or once-every-four-weeks (Q4W) dosing regimen of the PD-1 axis binding antagonist and the anti-TIGIT antagonist antibody can be administered in conjunction with one or more chemotherapeutic agents. The one or more chemotherapeutic agents can be administered at the same frequency as the frequency of administration of the PD-1 axis binding antagonist and the anti-TIGIT antagonist antibody (Q2W, Q3W, or Q4W) or at a different frequency (e.g., 3-weeks on/1-week off schedule). For example, in some embodiments, the PD-1 axis binding antagonist and the anti-TIGIT antagonist antibody are administered every two weeks and the one or more chemotherapeutic agents is administered every week, 3-weeks on/1-week off, every two weeks, every three weeks, or every four weeks. Alternatively, the PD-1 axis binding antagonist and the anti-TIGIT antagonist antibody are administered every three weeks and the one or more chemotherapeutic agents is administered every week, two weeks, every three weeks, or every four weeks. Alternatively, the PD-1 axis binding antagonist and the anti-TIGIT antagonist antibody are administered every four weeks and the one or more chemotherapeutic agents is administered every week, 3-weeks on/1-week off, every two weeks, every three weeks, or every four weeks. In certain instances, a chemotherapeutic agent is administered multiple times per week (e.g., 2, 3, 4, 5, 6 or 7 times per week (e.g., at Days 1, 2, and 3 of a dosing cycle)).

In some embodiments, the dose of a chemotherapeutic agent is reduced after one or more initial doses (e.g., after one, two, three, four, or more initial doses). For example, a subsequent dose of the chemotherapeutic agent (e.g., a platinum-based chemotherapeutic agent (e.g., carboplatin or cisplatin) and/or one or more non-platinum-based chemotherapeutic agents (e.g., an antimetabolite (e.g., pemetrexed or gemcitabine) can be administered at about 95%, 90%, 85%, 80%, 75%, 70%, 65%, 60%, 55%, 50%, 45%, 40%, 35%, 30%, 25%, 20%, 15%, 10%, or 5% of the initial dose. For example, an initial dose of cisplatin of about 75 mg/m² can be reduced for a subsequent dose, e.g., to 70 mg/m², 65 mg/m², 60 mg/m², 55 mg/m², 50 mg/m², or 45 mg/m²; an initial dose of pemetrexed of about 500 mg/m² can be reduced for a subsequent dose, e.g., to 450 mg/m², 400 mg/m², 350 mg/m², 300 mg/m², 250 mg/m², or 200 mg/m²; and/or an initial dose of carboplatin of a dose sufficient to achieve AUC=5 mg/ml/min can be reduced for a subsequent dose, e.g., to a dose sufficient to achieve AUC=4.5 mg/ml/min, 4.0 mg/ml/min, 3.5 mg/ml/min, or 3.0 mg/ml/min.

In some instances, administration of the effective amount of the anti-TIGIT antagonist antibody (e.g., an anti-TIGIT antagonist antibody as disclosed herein, e.g., tiragolumab), the PD-1 axis binding antagonist (e.g., anti-PD-L1 antagonist antibody, such as atezolizumab), the platinum-based chemotherapeutic agent (e.g., carboplatin or cisplatin), and the antimetabolite (e.g., pemetrexed) results in a CR or a PR. In some instances, administration of the effective amount of the anti-TIGIT antagonist antibody (e.g., an anti-TIGIT antagonist antibody as disclosed herein, e.g., tiragolumab), the PD-1 axis binding antagonist (e.g., anti-PD-L1 antagonist antibody, such as atezolizumab), the platinum-based chemotherapeutic agent (e.g., carboplatin or cisplatin), and the antimetabolite (e.g., pemetrexed) results in an increase in progression-free survival of the subject or population of subjects. In some instances, administration of the effective amount of the anti-TIGIT antagonist antibody (e.g., an anti-TIGIT antagonist antibody as disclosed herein, e.g., tiragolumab), the PD-1 axis binding antagonist (e.g., anti-PD-L1 antagonist antibody, such as atezolizumab), the platinum-based chemotherapeutic agent (e.g., carboplatin or cisplatin), and the antimetabolite (e.g., pemetrexed) extends OS of the subject or population of subjects.

In some instances, the subject or population of subjects receiving the anti-TIGIT antagonist antibody (e.g., an anti-TIGIT antagonist antibody as disclosed herein, e.g., tiragolumab), the PD-1 axis binding antagonist (e.g., anti-PD-L1 antagonist antibody, such as atezolizumab), the platinum-based chemotherapeutic agent (e.g., carboplatin or cisplatin), and the antimetabolite (e.g., pemetrexed) is being treated for a lung cancer, e.g., an NSCLC (e.g., non-squamous NSCLC (e.g., locally advanced unresectable or metastatic non-squamous NSCLC (e.g., Stage IV non-squamous NSCLC))).

Dosing of Agents

Dosing of anti-TIGIT antagonist antibodies, PD-1 axis binding antagonists, and chemotherapeutic agents is described in Section III(K).

Cancer Characterization

In any of the methods, uses, or compositions for use described herein, the lung cancer may be an NSCLC (e.g., non-squamous NSCLC (e.g., locally advanced unresectable or metastatic non-squamous NSCLC (e.g., Stage IV non-squamous NSCLC))). In some instances, the subject or population of subjects has not received prior systemic therapy for the lung cancer.

In some instances, in any of the methods, uses, or compositions for use described herein, the subject has no epidermal growth factor receptor (EGFR) or anaplastic lymphoma kinase (ALK) genomic tumor aberrations. In some instances, in any of the methods, uses, or compositions for use described herein, the subject does not have an EGFR gene mutation (e.g., a sensitizing EGFR gene mutation) or ALK gene rearrangement. In some instances, the subject has an Eastern Cooperative Oncology Group (ECOG) Performance Status (PS) of 0 or 1.

Methods for detecting the mutational status EGFR and ALK are well known in the art, and include, but are not limited to, sequencing DNA from clinical samples (e.g., tumor biopsies or blood samples (e.g., circulating tumor DNA in blood)) using a next-generation sequencing method, such as the targeted gene pulldown and sequencing method described in Frampton et al. (Nature Biotechnology. 31(11): 1023-1033, 2013), which is incorporated by reference herein in its entirety. Such a next-generation sequencing method can be used with any of the methods disclosed herein to detect various mutations (e.g., insertions, deletions, base substitutions, focal gene amplifications, and/or homozygous gene deletions), while enabling the use of small samples (e.g., from small-core needle biopsies, fine-needle aspirations, and/or cell blocks) or fixed samples (e.g., formalin-fixed and paraffin-embedded (FFPE) samples). Other methods for the detection of the mutational status of EGFR and ALK include fluorescence in situ hybridization (FISH) and immunohistochemical (IHC) methods. Exemplary methods for the detection of the mutational status of ALK are disclosed in U.S. Pat. No. 9,651,555, which is herein incorporated by reference in its entirety. In some instances, the VENTANA® anti-ALK (D5F3) IHC assay is used to determine the mutational status of the ALK gene.

In some instances of any of the methods described herein, the mutation is a sensitizing EGFR mutation. Sensitizing EGFR mutations are well known in the art and include those described in U.S. Publication No: US 2018/0235968 and in Juan et al. (Therapeutic Advances in Medical Oncology. 9(3): 201-216, 2017), which are incorporated by reference herein in their entireties. In some instances, the sensitizing EGFR mutation is a mutation in any one of exons 18-21 (e.g., a mutation in exon 18, exon 19, exon 20, and/or exon 21). In some instances, the sensitizing EGFR mutation is a deletion of exon 19 (dell 9). In other instances, sensitizing EGFR mutation is a L858R point mutation in exon 21. In some instances, the sensitizing EGFR mutation is a G719X point mutation in exon 18, wherein “X” is most commonly C, A, or S. In some instances, the sensitizing EGFR mutation is a G719S point mutation in exon 18. In some instances, the sensitizing EGFR mutation is a G719A point mutation in exon 18. In some instances, the sensitizing EGFR mutation is a S720F point mutation in exon 18. In some instances, the sensitizing EGFR mutation is a L861Q point mutation in exon 21. In some instances, the sensitizing EGFR mutation is a L861R point mutation in exon 21. In other instances, the sensitizing EGFR mutation is a T790M point mutation. In some instances, the sensitizing EGFR mutation is an E709X point mutation, where “X” is most commonly K, A, or H. In some instances, the sensitizing EGFR mutation is a S768I point mutation.

In some instances of any of the methods described herein, the mutation is an ALK gene rearrangement. ALK gene rearrangements are well known in the art and include those described in U.S. Pat. No. 9,651,555 and in Du et al. (Thoracic Cancer. 9: 423-430, 2018), which are incorporated herein by reference in their entireties. In some instances, the ALK gene rearrangement results in the creation of an oncogenic ALK tyrosine kinase that activates downstream signaling pathways resulting in increased cell proliferation and survival. In some instances, the ALK gene rearrangement is an ALK rearrangement with a gene selected from the group consisting of EML4, KIF5B, KLC1, TFG, TPR, HIP1, STRN, DCTN1, SQSTM1, NPM1, BCL11A, BIRC6, RANBP2, AT/C, CLTC, TMP4, and MSN resulting in the formation of a fusion oncogene. In some instances, the ALK gene rearrangement is an EML4 rearrangement with ALK resulting in the formation of the fusion oncogene EML4-ALK.

In some instances, in any of the methods, uses, or compositions for use described herein, the subject does not have a pulmonary lymphoepithelioma-like carcinoma subtype of NSCLC. Methods for detecting the subtype of NSCLC are well known in the art, and include, but are not limited to, methods of determination by histopathological criteria, or by molecular features (e.g., a subtype characterized by expression of one or a combination of biomarkers (e.g., particular genes or proteins encoded by said genes)). In some instances, the sample is selected from the group consisting of a tissue sample, a whole blood sample, a serum sample, and a plasma sample. In some instances, the tissue sample is a tumor sample.

In some instances, in any of the methods, uses, or compositions for use described herein, the subject does not have an active Epstein-Barr virus (EBV) infection or a known or suspected chronic active EBV infection. Indicators of active or chronic active EBV infections for use in the methods described herein can include, but are not limited to, EBV IgM, EBV IgG, Epstein-Barr nuclear antigen (EBNA), and Epstein-Barr viral particles detected in a sample from the subject (e.g., a blood or serum sample). Methods for detecting the presence of one or more indicators of active or chronic active EBV infection, including EBV IgM, EBV IgG, Epstein-Barr nuclear antigen (EBNA), and Epstein-Barr viral particles in a sample from a subject are well known in the art, and include, but are not limited to, methods involving serological diagnosis (e.g., the detection of EBV DNA (e.g., by PCR analysis of a blood sample for the detection of EBV viral particles) or EBV antigens or anti-EBV antibodies (e.g., detection of EBNA, EBV IgM, or EBV IgG using heterophilic antibodies). In some instances, the sample is selected from the group consisting of a whole blood sample, a serum sample, and a plasma sample. In some instances, the subject is negative for EBV IgM and/or negative by EBV PCR. In some instances, the subject is negative for EBV IgM and/or negative by EBV PCR and is positive for EBV IgG and/or positive for Epstein-Barr nuclear antigen (EBNA). In other instances, the subject is negative for EBV IgG and/or negative for EBNA.

In some instances, the subject has a PD-L1 selected tumor (e.g., a tumor PD-L1 expression with a minimum PD-L1-positive tumor cell fraction or TPS ≥30% (e.g., ≥50%) as determined by an IHC with the SP263 or 22C3 antibody or a proportion of tumor area occupied by PD-L1 expressing tumor-infiltrating immune cells (ICs) is greater than or equal to 1% in the tumor sample as determined by an IHC with the SP142 antibody). In some instances, the PD-L1 selected tumor is a tumor that has been determined to have a PD-L1-positive tumor cell fraction or PD-L1 TPS of greater than, or equal to, 30% (e.g., greater than, or equal to, 50%) by an immunohistochemical (IHC) assay. In some instances, the PD-L1 selected tumor is a tumor that has been determined to have a proportion of tumor area occupied by PD-L1 expressing immune cells (ICs) greater than or equal to 1% by an immunohistochemical (IHC) assay. In some instances, the IHC assay uses the anti-PD-L1 antibody SP263, 22C3, SP142, or 28-8. In some instances, the IHC assay uses anti-PD-L1 antibody SP263. In some instances, the IHC assay uses anti-PD-L1 antibody SP142. In some instances, the IHC assay uses anti-PD-L1 antibody 22C3. In some instances, the tumor sample has been determined to have a TPS of greater than, or equal to, 50%. In some instances, the PD-L1-positive tumor cell fraction is greater than, or equal to, 50% (e.g., as determined by positive staining with the anti-PD-L1 antibody SP263 (e.g., using the Ventana assay), as determined by positive staining with the anti-PD-L1 antibody 22C3 (e.g., using the pharmDx assay), or as determined by positive staining with the anti-PD-L1 antibody 28-8). In some embodiments, the PD-L1-positive tumor cell fraction is greater than, or equal to, 30%, as determined by positive staining with the anti-PD-L1 antibody SP142. In some instances, the ICs has been determined to be greater than, or equal to, 1% (e.g., as determined using the Ventana (SP142) PD-L1 IHC assay). In some instances, the ICs has been determined to be greater than, or equal to, 5% (e.g., as determined using the Ventana (SP142) PD-L1 IHC assay). In some instances, the ICs has been determined to be greater than, or equal to, 10% (e.g., as determined using the Ventana (SP142) PD-L1 IHC assay). In some instances, the ICs has been determined to be greater than, or equal to, 1% and less than 50% (e.g., as determined using the Ventana (SP142) PD-L1 IHC assay). In some instances, the ICs has been determined to be greater than, or equal to, 1% and less than 30% (e.g., as determined using the Ventana (SP142) PD-L1 IHC assay).

In some instances, in any of the methods, uses, or compositions for use described herein, a tumor sample obtained from the individual has a detectable protein expression level of PD-L1. In some instances, the detectable protein expression level of PD-L1 has been determined by an IHC assay. In some instances, the IHC assay uses anti-PD-L1 antibody SP142. In some instances, the tumor sample has been determined to have a detectable expression level of PD-L1 in greater than, or equal to, 1% of the tumor cells in the tumor sample. In some instances, the tumor sample has been determined to have a detectable expression level of PD-L1 in greater than, or equal to, 1% and less than 5% of the tumor cells in the tumor sample. In some instances, the tumor sample has been determined to have a detectable expression level of PD-L1 in greater than, or equal to, 5% and less than 50% of the tumor cells in the tumor sample. In some instances, the tumor sample has been determined to have a detectable expression level of PD-L1 in greater than, or equal to, 50% of the tumor cells in the tumor sample. In some instances, the tumor sample has been determined to have a detectable expression level of PD-L1 in tumor-infiltrating immune cells that comprise greater than, or equal to, 1% of the tumor sample. In some instances, the tumor sample has been determined to have a detectable expression level of PD-L1 in tumor-infiltrating immune cells that comprise greater than, or equal to, 1% and less than 5% of the tumor sample. In some instances, the tumor sample has been determined to have a detectable expression level of PD-L1 in tumor-infiltrating immune cells that comprise greater than, or equal to, 5% and less than 10% of the tumor sample. In some instances, the tumor sample has been determined to have a detectable expression level of PD-L1 in tumor-infiltrating immune cells that comprise greater than, or equal to, 10% of the tumor sample.

In some instances, the subject has a lung cancer (e.g., NSCLC (e.g., non-squamous NSCLC (e.g., locally advanced unresectable or metastatic non-squamous NSCLC (e.g., Stage IV non-squamous NSCLC)))) that has not been evaluated from PD-L1 expression. For example, in some instances, the subject having a lung cancer has not been determined to have a PD-L1-positive tumor cell fraction greater than, or equal to, 50% (e.g., the subject has not been determined to have a PD-L1-positive tumor cell fraction greater than, or equal to, 45%, 40%, 35%, or 30%). For example, in some instances, the subject has not been determined to have a TPS of greater than, or equal to, 50% PD-L1-positive (e.g., the subject has not been determined to have a TPS of greater than, or equal to, 45% PD-L1-positive, 40% PD-L1-positive, 35% PD-L1-positive, or 30% PD-L1-positive), e.g., as assessed using any of the IHC methods described herein or known in the art.

In some instances, the subject having a lung cancer (e.g., NSCLC (e.g., non-squamous NSCLC (e.g., locally advanced unresectable or metastatic non-squamous NSCLC (e.g., Stage IV non-squamous NSCLC)))) has been determined to have a PD-L1-positive tumor cell fraction of less than 50% (e.g., from 1% to 50%, from 1% to 49%, from 5% to 45%, from 10% to 40%, from 15% to 35%, or from 20% to 30%, e.g., from 1% to 5%, from 5% to 10%, from 10% to 15%, from 15% to 20%, from 20% to 25%, from 25% to 30%, from 30% to 35%, from 35% to 40%, from 40% to 45%, or from 45% to 49%, e.g., less than 49%, less than 45%, less than 40%, less than 35%, less than 30%, less than 25%, less than 20%, less than 15%, less than 10%, less than 9%, less than 8%, less than 7%, less than 6%, less than 5%, less than 4%, less than 3%, less than 2%, less than 1%, or about 0%). For example, in certain instances, the subject having a lung cancer has been determined to have a PD-L1-positive tumor cell fraction from 1-49% (e.g., from 1% to 5%, from 5% to 10%, from 10% to 15%, from 15% to 20%, from 20% to 25%, from 25% to 30%, from 30% to 35%, from 35% to 40%, from 40% to 45%, or from 45% to 49%). In other instances, the subject having a lung cancer has been determined to have a PD-L1-positive tumor cell fraction of less than 1% (e.g., about 0%, or an undetectable PD-L1 expression).

For example, in some instances, the subject having a lung cancer (e.g., NSCLC (e.g., non-squamous NSCLC (e.g., locally advanced unresectable or metastatic non-squamous NSCLC (e.g., Stage IV non-squamous NSCLC)))) has been determined to have a TPS of less than 50% PD-L1-positive (e.g., from 1% to 50%, from 1% to 49%, from 5% to 45%, from 10% to 40%, from 15% to 35%, or from 20% to 30% PD-L1-positive, e.g., from 1% to 5%, from 5% to 10%, from 10% to 15%, from 15% to 20%, from 20% to 25%, from 25% to 30%, from 30% to 35%, from 35% to 40%, from 40% to 45%, or from 45% to 49% PD-L1-positive, e.g., less than 49%, less than 45%, less than 40%, less than 35%, less than 30%, less than 25%, less than 20%, less than 15%, less than 10%, less than 9%, less than 8%, less than 7%, less than 6%, less than 5%, less than 4%, less than 3%, less than 2%, less than 1%, or about 0% PD-L1-positive). For example, in certain instances, the subject having a lung cancer has been determined to have a TPS from 1-49% PD-L1-positive (e.g., from 1% to 5%, from 5% to 10%, from 10% to 15%, from 15% to 20%, from 20% to 25%, from 25% to 30%, from 30% to 35%, from 35% to 40%, from 40% to 45%, or from 45% to 49% PD-L1-positive). In other instances, the subject having a lung cancer has been determined to have a TPS of less than 1% PD-L1-positive (e.g., about 0%, or an undetectable PD-L1 expression).

In particular embodiments, the subject has an NSCLC (e.g., non-squamous NSCLC (e.g., locally advanced unresectable or metastatic non-squamous NSCLC (e.g., Stage IV non-squamous NSCLC))) that has not been evaluated from PD-L1 expression. For example, in some instances, the subject having an NSCLC has not been determined to have a PD-L1-positive tumor cell fraction greater than, or equal to, 50% (e.g., the subject has not been determined to have a PD-L1-positive tumor cell fraction greater than, or equal to, 45%, 40%, 35%, or 30%). For example, in some instances, the subject has not been determined to have a TPS of greater than, or equal to, 50% PD-L1-positive (e.g., the subject has not been determined to have a TPS of greater than, or equal to, 45% PD-L1-positive, 40% PD-L1-positive, 35% PD-L1-positive, or 30% PD-L1-positive), e.g., as assessed using any of the IHC methods described herein or known in the art.

In some instances, the subject having an NSCLC (e.g., non-squamous NSCLC (e.g., locally advanced unresectable or metastatic non-squamous NSCLC (e.g., Stage IV non-squamous NSCLC))) has been determined to have a PD-L1-positive tumor cell fraction of less than 50% (e.g., from 1% to 50%, from 1% to 49%, from 5% to 45%, from 10% to 40%, from 15% to 35%, or from 20% to 30%, e.g., from 1% to 5%, from 5% to 10%, from 10% to 15%, from 15% to 20%, from 20% to 25%, from 25% to 30%, from 30% to 35%, from 35% to 40%, from 40% to 45%, or from 45% to 49%, e.g., less than 49%, less than 45%, less than 40%, less than 35%, less than 30%, less than 25%, less than 20%, less than 15%, less than 10%, less than 9%, less than 8%, less than 7%, less than 6%, less than 5%, less than 4%, less than 3%, less than 2%, less than 1%, or about 0%). For example, in certain instances, the subject having an NSCLC has been determined to have a PD-L1-positive tumor cell fraction from 1-49% (e.g., from 1% to 5%, from 5% to 10%, from 10% to 15%, from 15% to 20%, from 20% to 25%, from 25% to 30%, from 30% to 35%, from 35% to 40%, from 40% to 45%, or from 45% to 49%). In other instances, the subject having an NSCLC has been determined to have a PD-L1-positive tumor cell fraction of less than 1% (e.g., about 0%, or an undetectable PD-L1 expression).

For example, in some instances, the subject having an NSCLC (e.g., non-squamous NSCLC (e.g., locally advanced unresectable or metastatic non-squamous NSCLC (e.g., Stage IV non-squamous NSCLC))) has been determined to have a TPS of less than 50% PD-L1-positive (e.g., from 1% to 50%, from 1% to 49%, from 5% to 45%, from 10% to 40%, from 15% to 35%, or from 20% to 30% PD-L1-positive, e.g., from 1% to 5%, from 5% to 10%, from 10% to 15%, from 15% to 20%, from 20% to 25%, from 25% to 30%, from 30% to 35%, from 35% to 40%, from 40% to 45%, or from 45% to 49% PD-L1-positive, e.g., less than 49%, less than 45%, less than 40%, less than 35%, less than 30%, less than 25%, less than 20%, less than 15%, less than 10%, less than 9%, less than 8%, less than 7%, less than 6%, less than 5%, less than 4%, less than 3%, less than 2%, less than 1%, or about 0% PD-L1-positive). For example, in certain instances, the subject having an NSCLC has been determined to have a TPS from 1-49% PD-L1-positive (e.g., from 1% to 5%, from 5% to 10%, from 10% to 15%, from 15% to 20%, from 20% to 25%, from 25% to 30%, from 30% to 35%, from 35% to 40%, from 40% to 45%, or from 45% to 49% PD-L1-positive). In other instances, the subject having an NSCLC has been determined to have a TPS of less than 1% PD-L1-positive (e.g., about 0%, or an undetectable PD-L1 expression).

In some instances, a tumor sample obtained from the individual has a detectable nucleic acid expression level of PD-L1. In some instances, the detectable nucleic acid expression level of PD-L1 has been determined by RNA-seq, RT-qPCR, qPCR, multiplex qPCR or RT-qPCR, microarray analysis, SAGE, MassARRAY technique, ISH, or a combination thereof. In some instances, the sample is selected from the group consisting of a tissue sample, a whole blood sample, a serum sample, and a plasma sample. In some instances, the tissue sample is a tumor sample. In some instances, the tumor sample comprises tumor-infiltrating immune cells, tumor cells, stromal cells, and any combinations thereof.

In some instances, a tumor sample obtained from a subject having a lung cancer (e.g., NSCLC (e.g., non-squamous NSCLC (e.g., locally advanced unresectable or metastatic non-squamous NSCLC (e.g., Stage IV non-squamous NSCLC)))) has a low or undetectable nucleic acid expression level of PD-L1. In some instances, the nucleic acid expression level of PD-L1 has been determined by RNA-seq, RT-qPCR, qPCR, multiplex qPCR or RT-qPCR, microarray analysis, SAGE, MassARRAY technique, ISH, or a combination thereof. In some instances, the sample is selected from the group consisting of a tissue sample, a whole blood sample, a serum sample, and a plasma sample. In some instances, the tissue sample is a tumor sample. In some instances, the tumor sample comprises tumor-infiltrating immune cells, tumor cells, stromal cells, and any combinations thereof.

In some instances of any of the methods described herein, the subject having a lung cancer (e.g., NSCLC (e.g., non-squamous NSCLC (e.g., locally advanced unresectable or metastatic non-squamous NSCLC (e.g., Stage IV non-squamous NSCLC)))) has received no prior systemic treatment for the lung cancer (e.g., no prior systemic treatment with curative intent). In particular embodiments, the subject has a locally advanced lung cancer and has received no prior systemic treatment for the locally advanced lung cancer. In some instances, the subject has an NSCLC (e.g., a non-squamous NSCLC, e.g., a locally advanced unresectable or metastatic non-squamous NSCLC) and has received no prior systemic treatment for the NSCLC (e.g., a non-squamous NSCLC, e.g., a locally advanced unresectable or metastatic non-squamous NSCLC). Prior systemic treatments include prior neo-adjuvant, adjuvant chemotherapy, radiotherapy, and chemoradiotherapy with curative intent for non-metastatic disease.

In other instances, the subject having a lung cancer (e.g., NSCLC (e.g., non-squamous NSCLC (e.g., locally advanced unresectable or metastatic non-squamous NSCLC (e.g., Stage IV non-squamous NSCLC)))) has received prior systemic treatment for the lung cancer and has experienced a treatment-free interval of at least 12 months before treatment according to any of the methods of the present invention.

iv. Methods and Uses for Treating Resectable Lung Cancer

Provided herein are methods and uses for treating lung cancer (e.g., early stage lung cancer (e.g., resectable lung cancer (e.g., NSCLC (e.g., squamous or non-squamous NSCLC)))) in a subject comprising administering to the subject one or more dosing cycles of an effective amount of an anti-TIGIT antagonist antibody (e.g., an anti-TIGIT antagonist antibody as disclosed herein, e.g., tiragolumab) and a PD-1 axis binding antagonist (e.g., an anti-PD-L1 antagonist antibody, such as atezolizumab, or an anti-PD-1 antagonist antibody, such as pembrolizumab). In some instances, at least one dosing cycle is administered as a neoadjuvant treatment. In some instances, the treatment is a neoadjuvant treatment. In some instances, at least one dosing cycle is administered as an adjuvant treatment. In some instances, the treatment is an adjuvant treatment. In some instances, the treatment comprises a neoadjuvant treatment and an adjuvant treatment. In some instances, the lung cancer is a resectable lung cancer. In some instances, the lung cancer is an early stage lung cancer (e.g., stage II, IIIA, or IIIB lung cancer). In some instances, the lung cancer is an NSCLC (e.g., a squamous or non-squamous NSCLC). In some instances, the lung cancer is PD-L1 positive (e.g., PD-L1 high). In some instances, the lung cancer has no epidermal growth factor receptor (EGFR) or anaplastic lymphoma kinase (ALK) genomic tumor aberrations. In some embodiments, the subject has not been previously treated for lung cancer (e.g., a prior surgery, a prior immunotherapy, a prior chemotherapy, or a prior radiotherapy). In some instances, the subject is eligible to receive a platinum-based chemotherapy regimen. In some instances, the subject is eligible for an R0 resection with curative intent. The subject is preferably a human.

The present invention includes methods and uses for treating a subject having a resectable lung cancer (e.g., a resectable NSCLC (e.g., a resectable squamous or non-squamous NSCLC)), the method comprising administering to the subject one or more dosing cycles of an anti-TIGIT antagonist antibody (e.g., an anti-TIGIT antagonist antibody as disclosed herein, e.g., tiragolumab) at a dose (e.g., a fixed dose) of between about 30 mg to about 1200 mg every three weeks (e.g., on Day 1 of each 21-day dosing cycle) and a PD-1 axis binding antagonist (e.g., an anti-PD-L1 antagonist antibody, such as atezolizumab, or an anti-PD-1 antagonist antibody, such as e.g., pembrolizumab) at a dose (e.g., a fixed dose) of between about 80 mg to about 1600 mg every three weeks (e.g., on Day 1 of each 21-day dosing cycle). In some aspects, the method comprises administering to the subject one or more dosing cycles of an anti-TIGIT antagonist antibody (e.g., an anti-TIGIT antagonist antibody as disclosed herein, e.g., tiragolumab) at a dose (e.g., a fixed dose) of between 30 mg to 1200 mg every three weeks (e.g., on Day 1 of each 21-day dosing cycle) and a PD-1 axis binding antagonist (e.g., an anti-PD-L1 antagonist antibody, such as atezolizumab, or an anti-PD-1 antagonist antibody, such as e.g., pembrolizumab) at a dose (e.g., a fixed dose) of between 80 mg to 1600 mg every three weeks (e.g., on Day 1 of each 21-day dosing cycle).

The present invention includes methods and uses involving administration of an effective amount of an anti-TIGIT antagonist antibody (e.g., an anti-TIGIT antagonist antibody as disclosed herein, e.g., tiragolumab) and a PD-1 axis binding antagonist (e.g., an anti-PD-L1 antagonist antibody, such as atezolizumab, or an anti-PD-1 antagonist antibody, such as e.g., pembrolizumab) to a subject in need thereof every three weeks (e.g., on Day 1 of each 21-day dosing cycle). In some instances, at least one of the dosing cycles comprises administering to the subject the anti-TIGIT antagonist antibody at a dose (e.g., a fixed dose) of between about 30 mg to about 1200 mg every three weeks (e.g., a dose of 600 mg every three weeks) and the PD-1 axis binding antagonist at a dose (e.g., a fixed dose) of between about 80 mg to about 1600 mg every three weeks (e.g., a dose of about 1200 mg every three weeks) as a neoadjuvant treatment. In some instances, at least one of the dosing cycles comprises administering to the subject the anti-TIGIT antagonist antibody at a dose (e.g., a fixed dose) of between about 30 mg to about 1200 mg every three weeks (e.g., a dose of 600 mg every three weeks) and the PD-1 axis binding antagonist at a dose (e.g., a fixed dose) of between about 80 mg to about 1600 mg every three weeks (e.g., a dose of about 1200 mg every three weeks) as an adjuvant treatment. In some instances, at least one of the dosing cycles comprises administering to the subject the anti-TIGIT antagonist antibody at a dose (e.g., a fixed dose) of between 30 mg to 1200 mg every three weeks (e.g., a dose of 600 mg every three weeks) and the PD-1 axis binding antagonist at a dose (e.g., a fixed dose) of between 80 mg to 1600 mg every three weeks (e.g., a dose of 1200 mg every three weeks) as a neoadjuvant treatment. In some instances, at least one of the dosing cycles comprises administering to the subject the anti-TIGIT antagonist antibody at a dose (e.g., a fixed dose) of between 30 mg to 1200 mg every three weeks (e.g., a dose of 600 mg every three weeks) and the PD-1 axis binding antagonist at a dose (e.g., a fixed dose) of between 80 mg to 1600 mg every three weeks (e.g., a dose of 1200 mg every three weeks) as an adjuvant treatment.

In some instances, the subject receiving the anti-TIGIT antagonist antibody (e.g., an anti-TIGIT antagonist antibody as disclosed herein, e.g., tiragolumab) and the PD-1 axis binding antagonist (e.g., anti-PD-L1 antagonist antibody, such as atezolizumab) is being treated for a lung cancer (e.g., an early stage lung cancer (e.g., a resectable lung cancer (e.g., an NSCLC (e.g., a squamous or non-squamous NSCLC)))).

The PD-1 axis binding antagonist anti-TIGIT antagonist antibody may be administered in any suitable manner known in the art. For example, the PD-1 axis binding antagonist and anti-TIGIT antagonist antibody may be administered sequentially (on different days) or concurrently (on the same day or during the same treatment cycle). In some instances, the anti-TIGIT antagonist antibody and/or the PD-1 axis binding antagonist are administered on about Day 1 (e.g., Day −3, Day −2, Day −1, Day 1, Day 2, or Day 3) of a dosing cycle. In some instances, the PD-1 axis binding antagonist and anti-TIGIT antagonist antibody may be administered on the same day. In some instances, the PD-1 axis binding antagonist is administered before the anti-TIGIT antagonist antibody. In some instances, the PD-1 axis binding antagonist is administered after the anti-TIGIT antagonist antibody. In some instances, the PD-1 axis binding antagonist is administered simultaneously with the anti-TIGIT antagonist antibody. In some instances, the PD-1 axis binding antagonist may be administered prior to an anti-TIGIT antagonist antibody that is administered on the same day. In some instances, the PD-1 axis binding antagonist may be administered after to an anti-TIGIT antagonist antibody that is administered on the same day. In yet other instances, the PD-1 axis binding antagonist is administered at the same time as the anti-TIGIT antagonist antibody. In some instances, the PD-1 axis binding antagonist is in a separate composition as the anti-TIGIT antagonist antibody. In some instances, the PD-1 axis binding antagonist is in the same composition as the anti-TIGIT antagonist antibody. In some instances, the PD-1 axis binding antagonist is administered through a separate intravenous line from any other therapeutic agent administered to the patient on the same day. The PD-1 axis binding antagonist and anti-TIGIT antagonist antibody may be administered by the same route of administration or by different routes of administration. In some instances, the PD-1 axis binding antagonist is administered intravenously, intramuscularly, subcutaneously, topically, orally, transdermally, intraperitoneally, intraorbitally, by implantation, by inhalation, intrathecally, intraventricularly, or intranasally. In some instances, the PD-1 axis binding antagonist is administered intravenously. In some instances, the anti-TIGIT antagonist antibody is administered intravenously, intramuscularly, subcutaneously, topically, orally, transdermally, intraperitoneally, intraorbitally, by implantation, by inhalation, intrathecally, intraventricularly, or intranasally. In some instances, the anti-TIGIT antagonist antibody is administered intravenously. In some instances, there is a first observation period following administration of the PD-1 axis binding antagonist. In some instances, there is a second observation period following administration of the PD-1 axis binding antagonist. In some instances, there is a first observation period following administration of the anti-TIGIT antagonist antibody. In some instances, there is a second observation period following administration of the anti-TIGIT antagonist antibody. In some instances, the observation period is between about 30 minutes to about 60 minutes in length. In some instances, the anti-TIGIT antagonist antibody and/or PD-1 axis binding antagonist are administered intravenously or subcutaneously. In some instances, the intravenous infusion is over 30±10 minutes and/or over 60±15 minutes. In one example, atezolizumab may be administered intravenously over 60 minutes; if the first infusion is tolerated, all subsequent infusions may be delivered over 30 minutes. In some examples, the PD-1 axis binding antagonist is not administered as an intravenous push or bolus. In one example, tiragolumab may be administered intravenously over 60 minutes; if the first infusion is tolerated, all subsequent infusions may be delivered over 30 minutes. In some examples, the anti-TIGIT antagonist antibody is not administered as an intravenous push or bolus.

In any of the preceding examples, each dosing cycle may have any suitable length, e.g., about 7 days (about 5, 6, 7, 8, or 9 days), about 14 days (e.g., about 12, 13, 14, 15, or 16 days), about 21 days (e.g., about 18, 19, 20, 21, 22, 23, or 24 days), about 28 days (about 25, 26, 27, 28, 29, 30, or 31 days), or longer. In some instances, each dosing cycle is about 21 days.

In some instances, a PD-L1 expression level of a sample (e.g., a tumor sample, a blood sample (e.g., a plasma sample), or a lymph sample) obtained from the subject has been determined. In some instances, the sample has been determined to have a detectable expression level of PD-L1 (e.g., a detectable protein and/or nucleic acid expression level of PD-L1). In some instances, the detectable expression level of PD-L1 is a detectable protein expression level of PD-L1. In some instances, the detectable expression level of PD-L1 is a PD-L1-positive tumor cell fraction (e.g., a PD-L1-positive tumor cell fraction of greater than or equal to 50%). In some instances, the detectable protein expression level of PD-L1 has been determined by an immunohistochemical (IHC) assay comprising staining with an anti-PD-L1 antibody suitable for staining (e.g., anti-PD-L1 antibody SP263). In some instances, the IHC assay is a Ventana SP263 IHC assay. In some instances, the detectable expression level of PD-L1 is a detectable nucleic acid expression level of PD-L1. In some instances, the mutational status of epidermal growth factor receptor (EGFR) or anaplastic lymphoma kinase (ALK) is determined. In some instances, the method further comprises obtaining a sample from the subject. In some instances, the method further comprises determining the expression level of PD-L1.

In some instances, the first dosing cycle is initiated prior to a surgery (e.g., a segmentectomy, a lobectomy, a bilobectomy, or a pneumonectomy). In some instances, one or more dosing cycles are completed prior to a surgery. In some instances, at least 1, 2, 3, or 4 dosing cycles (e.g., 1, 2, 3, 4, 5, 6, 7, 8, or more dosing cycles) are completed prior to a surgery. In some instances, 4 dosing cycles are completed prior to a surgery. In some instances, one or more dosing cycles (e.g., 1, 2, 3, 4, 5, 6, 7, 8, 9, 10, 11, 12, 13, 14, 15, 16, 17, 18, 19, 20, or more dosing cycles) are initiated after a surgery. In some instances, 16 dosing cycles are completed after a surgery. In some instances, the treatment includes a surgery. In some instances, the surgery is a segmentectomy, a lobectomy, a bilobectomy, or a pneumonectomy. In some instances, the treatment includes a radiotherapy (e.g., a post-operative radiotherapy).

In some instances, the treating results in an increase in major pathological response (MPR) rate as compared to a reference MPR rate. In some instances, the treating results in a pathological complete response (pCR) and/or an increase in pCR rate as compared to a reference pCR rate. In some instances, the treating results in an increase in event-free survival (EFS) as compared to a reference EFS time. In some instances, the treating results in an increase in OS as compared to a reference OS time. In some instances, the reference MPR rate, reference pCR rate, and/or reference EFS time are an MPR rate, a pCR rate, and/or an EFS time of a population of subjects who have received a treatment comprising: (a) a PD-1 axis binding antagonist without an anti-TIGIT antagonist antibody; and/or (b) cisplatin and docetaxel or cisplatin, docetaxel, and bevacizumab. In some instances, administration of the effective amount of the anti-TIGIT antagonist antibody (e.g., an anti-TIGIT antagonist antibody as disclosed herein, e.g., tiragolumab) and the PD-1 axis binding antagonist (e.g., an anti-PD-L1 antagonist antibody, such as atezolizumab) results in an increase in MPR rate. In some instances, administration of the effective amount of the anti-TIGIT antagonist antibody (e.g., an anti-TIGIT antagonist antibody as disclosed herein, e.g., tiragolumab) and the PD-1 axis binding antagonist (e.g., an anti-PD-L1 antagonist antibody, such as atezolizumab) results in a pCR. In some instances, administration of the effective amount of the anti-TIGIT antagonist antibody (e.g., an anti-TIGIT antagonist antibody as disclosed herein, e.g., tiragolumab) and the PD-1 axis binding antagonist (e.g., an anti-PD-L1 antagonist antibody, such as atezolizumab) results in an increase in EFS. In some instances, administration of the effective amount of the anti-TIGIT antagonist antibody (e.g., an anti-TIGIT antagonist antibody as disclosed herein, e.g., tiragolumab) and the PD-1 axis binding antagonist (e.g., an anti-PD-L1 antagonist antibody, such as atezolizumab) results in an increase in OS.

In some instances, the treatment further comprises one or more chemotherapeutic agents (e.g., a platinum-based chemotherapeutic agent (e.g., carboplatin or cisplatin) and/or one or more non-platinum-based chemotherapeutic agents (e.g., an antimetabolite (e.g., pemetrexed or gemcitabine) and/or a taxane (e.g., paclitaxel, e.g., nab-paclitaxel)). In some instances, the neoadjuvant and/or adjuvant treatment further comprises one or more chemotherapeutic agents (e.g., a platinum-based chemotherapeutic agent (e.g., carboplatin or cisplatin) and/or one or more non-platinum-based chemotherapeutic agents (e.g., an antimetabolite (e.g., pemetrexed or gemcitabine) and/or a taxane (e.g., paclitaxel, e.g., nab-paclitaxel)). In some instances, the one or more chemotherapeutic agents are one or more platinum-based chemotherapeutic agents and/or one or more non-platinum-based chemotherapeutic agents. In some instances, the platinum-based chemotherapeutic agent is carboplatin or cisplatin. In some instances, the non-platinum-based chemotherapeutic agents are an antimetabolite (e.g., pemetrexed or gemcitabine) and/or a taxane (e.g., paclitaxel, e.g., nab-paclitaxel). In some instances, the one or more chemotherapeutic agents are a platinum-based chemotherapeutic agent (e.g., carboplatin or cisplatin) and a non-platinum-based chemotherapeutic agent (e.g., an antimetabolite (e.g., pemetrexed or gemcitabine) and/or a taxane (e.g., paclitaxel, e.g., nab-paclitaxel)). In some instances, at least one or more chemotherapeutic agents are: (a) carboplatin and pemetrexed; (b) carboplatin and paclitaxel; (c) cisplatin and pemetrexed; (d) carboplatin and gemcitabine; or (e) cisplatin and gemcitabine. In some instances, the one or more chemotherapeutic agents used in a treatment for non-squamous NSCLC are (a) carboplatin and pemetrexed, (b) carboplatin and paclitaxel, or (c) cisplatin and pemetrexed. In some instances, the one or more chemotherapeutic agents used in a treatment for squamous NSCLC are (a) carboplatin and gemcitabine, (b) carboplatin and paclitaxel, or (c) cisplatin and gemcitabine. In some instances, the treatment further comprises administering one or more chemotherapeutic agents (e.g., a platinum-based chemotherapeutic agent (e.g., carboplatin or cisplatin) and/or one or more non-platinum-based chemotherapeutic agents (e.g., an antimetabolite (e.g., pemetrexed or gemcitabine) and/or a taxane (e.g., paclitaxel, e.g., nab-paclitaxel)). In some instances, the one or more chemotherapeutic agents are administered every three weeks. In some instances, the one or more chemotherapeutic agents are administered on about Day 1 (e.g., Day −3, Day −2, Day −1, Day 1, Day 2, or Day 3) of one or more dosing cycles. In some instances, the one or more chemotherapeutic agents are administered on about Day 1 (e.g., Day −3, Day −2, Day −1, Day 1, Day 2, or Day 3) and on about Day 8 (e.g., Day 5, Day 6, Day 7, Day 8, Day 9, Day 10, or Day 11) of one or more dosing cycles. In some instances, the dosing cycles are, e.g., about 7 days (about 5, 6, 7, 8, or 9 days), about 14 days (e.g., about 12, 13, 14, 15, or 16 days), about 21 days (e.g., about 18, 19, 20, 21, 22, 23, or 24 days), about 28 days (about 25, 26, 27, 28, 29, 30, or 31 days), or longer. In some instances, each dosing cycle is about 21 days. In some instances, the one or more chemotherapeutic agents are administered intravenously, intramuscularly, subcutaneously, topically, orally, transdermally, intraperitoneally, intraorbitally, by implantation, by inhalation, intrathecally, intraventricularly, or intranasally. In some instances, the one or more chemotherapeutic agents are administered after the PD-1 axis binding antagonist (e.g., atezolizumab) and/or anti-TIGIT antagonist antibody (e.g., an anti-TIGIT antagonist antibody as disclosed herein, e.g., tiragolumab). In some instances, the non-platinum-based chemotherapeutic agent (e.g., an antimetabolite (e.g., pemetrexed or gemcitabine) and/or a taxane (e.g., paclitaxel, e.g., nab-paclitaxel)) is administered before the platinum-based chemotherapeutic agent (e.g., carboplatin or cisplatin).

The present invention includes methods and uses for treating a subject having a resectable lung cancer (e.g., an early stage resectable lung cancer (e.g., a resectable NSCLC (e.g., a resectable squamous or non-squamous NSCLC))), the method comprising administering to the subject one or more dosing cycles of an anti-TIGIT antagonist antibody (e.g., an anti-TIGIT antagonist antibody as disclosed herein, e.g., tiragolumab) at a dose (e.g., a fixed dose) of between about 30 mg to about 600 mg every three weeks, a PD-1 axis binding antagonist (e.g., an anti-PD-L1 antagonist antibody, such as atezolizumab, or an anti-PD-1 antagonist antibody, such as e.g., pembrolizumab) at a dose (e.g., a fixed dose) of between about 80 mg to about 1600 mg every three weeks, a platinum-based chemotherapeutic agent (e.g., cisplatin or carboplatin), and a non-platinum-based chemotherapeutic agent (e.g., an antimetabolite (e.g., pemetrexed or gemcitabine) or a taxane (e.g., paclitaxel or nab-paclitaxel)). In some aspects, the method comprising administering to the subject one or more dosing cycles of an anti-TIGIT antagonist antibody (e.g., an anti-TIGIT antagonist antibody as disclosed herein, e.g., tiragolumab) at a dose (e.g., a fixed dose) of between 30 mg to 600 mg every three weeks, a PD-1 axis binding antagonist (e.g., an anti-PD-L1 antagonist antibody, such as atezolizumab, or an anti-PD-1 antagonist antibody, such as e.g., pembrolizumab) at a dose (e.g., a fixed dose) of between 80 mg to 1600 mg every three weeks, a platinum-based chemotherapeutic agent (e.g., cisplatin or carboplatin), and a non-platinum-based chemotherapeutic agent (e.g., an antimetabolite (e.g., pemetrexed or gemcitabine) or a taxane (e.g., paclitaxel or nab-paclitaxel)).

The present invention includes methods and uses for treating a subject having a lung cancer (e.g., an early stage lung cancer (e.g., a resectable lung cancer (e.g., an NSCLC (e.g., a squamous or non-squamous NSCLC)))), the method comprising administering to the subject one or more dosing cycles of an anti-TIGIT antagonist antibody (e.g., an anti-TIGIT antagonist antibody as disclosed herein, e.g., tiragolumab), a PD-1 axis binding antagonist (e.g., an anti-PD-L1 antagonist antibody, such as atezolizumab, or an anti-PD-1 antagonist antibody, such as e.g., pembrolizumab), a platinum-based chemotherapeutic agent (e.g., cisplatin or carboplatin), and a non-platinum-based chemotherapeutic agent (e.g., an antimetabolite (e.g., pemetrexed or gemcitabine) or a taxane (e.g., paclitaxel or nab-paclitaxel)), wherein at least one of the dosing cycles comprises administering to the subject the anti-TIGIT antagonist antibody at a dose (e.g., a fixed dose) of between about 30 mg to about 600 mg every three weeks, the PD-1 axis binding antagonist at a dose (e.g., a fixed dose) of between about 80 mg to about 1600 mg every three weeks, a platinum-based chemotherapeutic agent (e.g., cisplatin or carboplatin), and a non-platinum-based chemotherapeutic agent (e.g., an antimetabolite (e.g., pemetrexed or gemcitabine) or a taxane (e.g., paclitaxel or nab-paclitaxel)) as a neoadjuvant treatment. In some aspects, at least one of the dosing cycles comprises administering to the subject the anti-TIGIT antagonist antibody at a dose (e.g., a fixed dose) of between 30 mg to 600 mg every three weeks, the PD-1 axis binding antagonist at a dose (e.g., a fixed dose) of between 80 mg to 1600 mg every three weeks, a platinum-based chemotherapeutic agent (e.g., cisplatin or carboplatin), and a non-platinum-based chemotherapeutic agent (e.g., an antimetabolite (e.g., pemetrexed or gemcitabine) or a taxane (e.g., paclitaxel or nab-paclitaxel)) as a neoadjuvant treatment.

The present invention includes methods and uses for treating a subject having a resectable lung cancer (e.g., an early stage resectable lung cancer (e.g., a resectable NSCLC (e.g., a resectable squamous or non-squamous NSCLC))), the method comprising administering to the subject one or more dosing cycles of an anti-TIGIT antagonist antibody (e.g., an anti-TIGIT antagonist antibody as disclosed herein, e.g., tiragolumab) at a dose (e.g., a fixed dose) of between about 30 mg to about 600 mg every three weeks, a PD-1 axis binding antagonist (e.g., an anti-PD-L1 antagonist antibody, such as atezolizumab, or an anti-PD-1 antagonist antibody, such as e.g., pembrolizumab) at a dose (e.g., a fixed dose) of between about 80 mg to about 1600 mg every three weeks, and: (a) (i) a platinum-based chemotherapeutic agent (e.g., cisplatin or carboplatin) at a dose targeted to achieve an AUC of 5 mg/mL/min or an AUC of 6 mg/mL/min every three weeks; or (ii) a platinum-based chemotherapeutic agent (e.g., cisplatin or carboplatin) at a dose of about 75 mg/m² every three weeks; and (b) (i) an antimetabolite at a dose of about 500 mg/m² every three weeks or about 1000 mg/m² or about 1250 mg/m² on Days 1 and 8 of each dosing cycle; or (ii) a taxane at a dose of about 100 mg/m², about 175 mg/m², or about 200 mg/m² every three weeks. In some aspects, the method comprises administering to the subject one or more dosing cycles of an anti-TIGIT antagonist antibody (e.g., an anti-TIGIT antagonist antibody as disclosed herein, e.g., tiragolumab) at a dose (e.g., a fixed dose) of between 30 mg to 600 mg every three weeks, a PD-1 axis binding antagonist (e.g., an anti-PD-L1 antagonist antibody, such as atezolizumab, or an anti-PD-1 antagonist antibody, such as e.g., pembrolizumab) at a dose (e.g., a fixed dose) of between 80 mg to 1600 mg every three weeks, and: (a) (i) a platinum-based chemotherapeutic agent (e.g., cisplatin or carboplatin) at a dose targeted to achieve an AUC of 5 mg/mL/min or an AUC of 6 mg/mL/min every three weeks; or (ii) a platinum-based chemotherapeutic agent (e.g., cisplatin or carboplatin) at a dose of 75 mg/m² every three weeks; and (b) (i) an antimetabolite at a dose of 500 mg/m² every three weeks or 1000 mg/m² or 1250 mg/m² on Days 1 and 8 of each dosing cycle; or (ii) a taxane at a dose of 100 mg/m², 175 mg/m², or 200 mg/m² every three weeks.

The present invention includes methods and uses for treating a subject having a lung cancer (e.g., an early stage lung cancer (e.g., a resectable lung cancer (e.g., an NSCLC (e.g., a squamous or non-squamous NSCLC)))), the method comprising administering to the subject one or more dosing cycles of an anti-TIGIT antagonist antibody (e.g., an anti-TIGIT antagonist antibody as disclosed herein, e.g., tiragolumab), a PD-1 axis binding antagonist (e.g., an anti-PD-L1 antagonist antibody, such as atezolizumab, or an anti-PD-1 antagonist antibody, such as e.g., pembrolizumab), a platinum-based chemotherapeutic agent (e.g., cisplatin or carboplatin), and a non-platinum-based chemotherapeutic agent (e.g., an antimetabolite (e.g., pemetrexed or gemcitabine) or a taxane (e.g., paclitaxel or nab-paclitaxel)), wherein at least one of the dosing cycles comprises administering to the subject: (a) the anti-TIGIT antagonist antibody at a dose (e.g., a fixed dose) of between about 30 mg to about 600 mg every three weeks; (b) the PD-1 axis binding antagonist at a dose (e.g., a fixed dose) of between about 80 mg to about 1600 mg every three weeks; (c) the platinum-based chemotherapeutic agent: (i) at a dose targeted to achieve an AUC of 5 mg/mL/min or an AUC of 6 mg/mL/min every three weeks; or (ii) at a dose of about 75 mg/m² every three weeks; and (d) the non-platinum-based chemotherapeutic agent, wherein the non-platinum-based chemotherapeutic agent is: (i) an antimetabolite at a dose of about 500 mg/m² every three weeks or about 1000 mg/m² or about 1250 mg/m² on Days 1 and 8 of each dosing cycle; or (ii) a taxane at a dose of about 100 mg/m², about 175 mg/m², or about 200 mg/m² every three weeks; wherein the treatment is a neoadjuvant treatment. In some aspects, at least one of the dosing cycles comprises administering to the subject: (a) the anti-TIGIT antagonist antibody at a dose (e.g., a fixed dose) of between 30 mg to 600 mg every three weeks; (b) the PD-1 axis binding antagonist at a dose (e.g., a fixed dose) of between 80 mg to 1600 mg every three weeks; (c) the platinum-based chemotherapeutic agent: (i) at a dose targeted to achieve an AUC of 5 mg/mL/min or an AUC of 6 mg/mL/min every three weeks; or (ii) at a dose of 75 mg/m² every three weeks; and (d) the non-platinum-based chemotherapeutic agent, wherein the non-platinum-based chemotherapeutic agent is: (i) an antimetabolite at a dose of 500 mg/m² every three weeks or 1000 mg/m² or 1250 mg/m² on Days 1 and 8 of each dosing cycle; or (ii) a taxane at a dose of 100 mg/m², 175 mg/m², or 200 mg/m² every three weeks; wherein the treatment is a neoadjuvant treatment.

The present invention includes methods and uses for treating a subject having a lung cancer (e.g., an early stage lung cancer (e.g., a resectable lung cancer (e.g., an NSCLC (e.g., a squamous or non-squamous NSCLC)))), the method comprising administering to the subject one or more dosing cycles of an anti-TIGIT antagonist antibody and a PD-1 axis binding antagonist, wherein: (a) at least one of the dosing cycles comprises administering to the subject the anti-TIGIT antagonist antibody at a dose (e.g., a fixed dose) of between about 30 mg to about 1200 mg every three weeks and the PD-1 axis binding antagonist at a dose (e.g., a fixed dose) of between about 80 mg to about 1600 mg every three weeks as a neoadjuvant treatment; and (b) at least one of the dosing cycles comprises administering to the subject the anti-TIGIT antagonist antibody at a dose (e.g., a fixed dose) of between about 30 mg to about 1200 mg every three weeks and the PD-1 axis binding antagonist at a dose (e.g., a fixed dose) of between about 80 mg to about 1600 mg every three weeks as an adjuvant treatment. In some aspects, the method comprises administering to the subject one or more dosing cycles of an anti-TIGIT antagonist antibody and a PD-1 axis binding antagonist, wherein: (a) at least one of the dosing cycles comprises administering to the subject the anti-TIGIT antagonist antibody at a dose (e.g., a fixed dose) of between 30 mg to 1200 mg every three weeks and the PD-1 axis binding antagonist at a dose (e.g., a fixed dose) of between 80 mg to 1600 mg every three weeks as a neoadjuvant treatment; and (b) at least one of the dosing cycles comprises administering to the subject the anti-TIGIT antagonist antibody at a dose (e.g., a fixed dose) of between 30 mg to 1200 mg every three weeks and the PD-1 axis binding antagonist at a dose (e.g., a fixed dose) of between 80 mg to 1600 mg every three weeks as an adjuvant treatment.

The present invention includes methods and uses for treating a subject having a lung cancer (e.g., an early stage lung cancer (e.g., a resectable lung cancer (e.g., an NSCLC (e.g., a squamous or non-squamous NSCLC)))), the method comprising administering to the subject one or more dosing cycles of an anti-TIGIT antagonist antibody and a PD-1 axis binding antagonist, wherein: (I) at least one of the dosing cycles is a neoadjuvant treatment and comprises administering to the subject: (a) the anti-TIGIT antagonist antibody at a dose (e.g., a fixed dose) of between about 30 mg to about 1200 mg every three weeks; (b) the PD-1 axis binding antagonist at a dose (e.g., a fixed dose) of between about 80 mg to about 1600 mg every three weeks as a neoadjuvant treatment; (c) a platinum-based chemotherapeutic agent: (i) at a dose targeted to achieve an AUC of 5 mg/mL/min or an AUC of 6 mg/mL/min every three weeks; or (ii) at a dose of about 75 mg/m² every three weeks; and (d) a non-platinum-based chemotherapeutic agent, wherein the non-platinum-based chemotherapeutic agent is: (i) an antimetabolite at a dose of about 500 mg/m² every three weeks or about 1000 mg/m² or about 1250 mg/m² on Days 1 and 8 of each dosing cycle; or (ii) a taxane at a dose of about 100 mg/m², about 175 mg/m², or about 200 mg/m² every three weeks; and (II) at least one of the dosing cycles comprises administering to the subject the anti-TIGIT antagonist antibody at a dose (e.g., a fixed dose) of between about 30 mg to about 1200 mg every three weeks and the PD-1 axis binding antagonist at a dose (e.g., a fixed dose) of between about 80 mg to about 1600 mg every three weeks as an adjuvant treatment. In some aspects, the method comprises administering to the subject: (a) the anti-TIGIT antagonist antibody at a dose (e.g., a fixed dose) of between 30 mg to 1200 mg every three weeks; (b) the PD-1 axis binding antagonist at a dose (e.g., a fixed dose) of between 80 mg to 1600 mg every three weeks as a neoadjuvant treatment; (c) a platinum-based chemotherapeutic agent: (i) at a dose targeted to achieve an AUC of 5 mg/mL/min or an AUC of 6 mg/mL/min every three weeks; or (ii) at a dose of 75 mg/m² every three weeks; and (d) a non-platinum-based chemotherapeutic agent, wherein the non-platinum-based chemotherapeutic agent is: (i) an antimetabolite at a dose of 500 mg/m² every three weeks or 1000 mg/m² or 1250 mg/m² on Days 1 and 8 of each dosing cycle; or (ii) a taxane at a dose of 100 mg/m², 175 mg/m², or 200 mg/m² every three weeks; and (II) at least one of the dosing cycles comprises administering to the subject the anti-TIGIT antagonist antibody at a dose (e.g., a fixed dose) of between 30 mg to 1200 mg every three weeks and the PD-1 axis binding antagonist at a dose (e.g., a fixed dose) of between 80 mg to 1600 mg every three weeks as an adjuvant treatment.

In some instances, the treatment may further comprise an additional therapy. Any suitable additional therapy known in the art or described herein may be used. The additional therapy may be radiation therapy (e.g., a post-operative radiotherapy), surgery, gene therapy, DNA therapy, viral therapy, RNA therapy, immunotherapy, bone marrow transplantation, nanotherapy, monoclonal antibody therapy, gamma irradiation, or a combination of the foregoing.

In some instances, the additional therapy is the administration of side-effect limiting agents (e.g., agents intended to lessen the occurrence and/or severity of side effects of treatment, such as anti-nausea agents, a corticosteroid (e.g., prednisone or an equivalent, e.g., at a dose of 1-2 mg/kg/day), hormone replacement medicine(s), and the like).

Also provided herein are methods for treating lung cancer in a subject comprising administering to the subject a treatment regimen comprising an effective amount of a PD-1 axis binding antagonist (e.g., atezolizumab) and/or anti-TIGIT antagonist antibody (e.g., an anti-TIGIT antagonist antibody as disclosed herein, e.g., tiragolumab) in combination with one or more chemotherapeutic agents (e.g., a platinum-based chemotherapeutic agent (e.g., carboplatin or cisplatin) and/or one or more non-platinum-based chemotherapeutic agents (e.g., an antimetabolite (e.g., pemetrexed or gemcitabine) and/or a taxane (e.g., paclitaxel, e.g., nab-paclitaxel))) and/or cancer therapy (e.g., a surgery and/or a radiotherapy). For example, a PD-1 axis binding antagonist may be administered in combination with an additional chemotherapy or chemotherapeutic agent (see definition above); a targeted therapy or targeted therapeutic agent; an immunotherapy or immunotherapeutic agent, for example, a monoclonal antibody; one or more cytotoxic agents (see definition above); or combinations thereof.

Dosing of Agents

Dosing of anti-TIGIT antagonist antibodies, PD-1 axis binding antagonists, and chemotherapeutic agents is described in Section III(K).

Cancer Characterization and Selection

In some instances, in any of the methods, uses, or compositions for use described herein, the lung cancer (e.g., early-stage lung cancer (e.g., resectable lung cancer (e.g., NSCLC (e.g., squamous or non-squamous NSCLC)))) is resectable (e.g., eligible for R0 resection with curative intent). In some instances, the lung cancer is an NSCLC. In some instances, the NSCLC is a squamous or non-squamous NSCLC. In some instances, the lung cancer is PD-L1 positive. In some instances, the lung cancer is PD-L1 high. In some instances, the lung cancer has no epidermal growth factor receptor (EGFR) or anaplastic lymphoma kinase (ALK) genomic tumor aberrations. In some instances, the lung cancer is a stage II, IIIA, or IIIB lung cancer.

In some instances, in any of the methods, uses, or compositions for use described herein, the subject is eligible for platinum-based chemotherapy. In some instances, the subject has an Eastern Cooperative Oncology Group (ECOG) Performance Status (PS) of 0 or 1. In some instances, the subject has not received a prior therapy (e.g., an immunotherapy, a chemotherapy, or a radiotherapy) for lung cancer.

In some instances, in any of the methods, uses, or compositions for use described herein, the presence or level of circulating tumor DNA (ctDNA) may be assessed. In some instances, ctDNA is assessed in a sample (e.g., a blood sample (e.g., a plasma sample)) from the subject. In some instances, ctDNA is assessed in a sample from the subject prior to day 1 of the first dosing cycle (e.g., the first dosing cycle of an anti-TIGIT antagonist antibody and a PD-1 axis binding antagonist). In some instances, ctDNA is assessed in a sample from the subject prior to surgery. In some instances, ctDNA is assessed in a sample from the subject after surgery.

In some instances, in any of the methods, uses, or compositions for use described herein, the presence or level of immune cells (e.g., T cells) may be assessed. In some instances, the presence or level of immune cells is assessed in a sample (e.g., a blood sample, a tumor tissue sample, or a lymph node sample) from the subject. In some instances, the presence or level of immune cells is assessed in a sample from the subject prior to surgery. In some instances, the presence or level of immune cells is assessed in a sample from the subject after surgery.

Assessment of PD-L1 Expression

The expression of PD-L1 may be assessed as described in Section III(L).

Assessment of EGFR and ALK Aberrations

Methods for detecting the mutational status EGFR and ALK are described in Section III(N) herein.

Responses to Treatment

In some embodiments of any of the methods described herein, a subject's response to the therapy can be characterized by one or more measures. In some embodiments, the treatment results in an increase in major pathological response (MPR) rate. In some embodiments, the treatment results in a pCR. In some embodiments, the treatment results in an increase in event-free survival (EFS). In some embodiments, the treatment results in an improvement in patient-reported outcomes. In some embodiments, the treatment results in an improvement in patient-reported physical functioning, role functioning, or GHS/QoL, as measured by the EORTC-QLQ-C30. In some embodiments, the treatment results in an improvement in patient-reported lung cancer symptoms for cough, dyspnea, and chest pain, as measured through the use of the EORTC-QLQ-LC13.

In some instances, the treatment results in an increase in MPR rate of the subject, e.g., as compared to treatment with the PD-1 axis binding antagonist without the anti-TIGIT antagonist antibody or as compared to treatment with the anti-TIGIT antagonist antibody without the PD-1 axis binding antagonist. In some instances, the treatment results in an increase in MPR rate of the subject, e.g., as compared to treatment with the PD-1 axis binding antagonist and the anti-TIGIT antagonist antibody without chemotherapy (e.g., a platinum-based doublet chemotherapy (e.g., a platinum-based chemotherapeutic agent (e.g., carboplatin or cisplatin) and a non-platinum-based chemotherapeutic agent (e.g., an antimetabolite (e.g., pemetrexed or gemcitabine) or a taxane (e.g., paclitaxel or nab-paclitaxel)))).

In some instances, the treatment results in an increase in pCR of the subject, e.g., as compared to treatment with the PD-1 axis binding antagonist without the anti-TIGIT antagonist antibody or as compared to treatment with the anti-TIGIT antagonist antibody without the PD-1 axis binding antagonist. In some instances, the treatment results in an increase in pCR of the subject, e.g., as compared to treatment with the PD-1 axis binding antagonist and the anti-TIGIT antagonist antibody without chemotherapy (e.g., a platinum-based doublet chemotherapy (e.g., a platinum-based chemotherapeutic agent (e.g., carboplatin or cisplatin) and a non-platinum-based chemotherapeutic agent (e.g., an antimetabolite (e.g., pemetrexed or gemcitabine) or a taxane (e.g., paclitaxel or nab-paclitaxel)))).

In some instances, the treatment extends OS of the subject, e.g., as compared to treatment with the PD-1 axis binding antagonist without the anti-TIGIT antagonist antibody or as compared to treatment with the anti-TIGIT antagonist antibody without the PD-1 axis binding antagonist. In some instances, the treatment extends OS of the subject, e.g., as compared to treatment with the PD-1 axis binding antagonist and the anti-TIGIT antagonist antibody without chemotherapy (e.g., a platinum-based doublet chemotherapy (e.g., a platinum-based chemotherapeutic agent (e.g., carboplatin or cisplatin) and a non-platinum-based chemotherapeutic agent (e.g., an antimetabolite (e.g., pemetrexed or gemcitabine) or a taxane (e.g., paclitaxel or nab-paclitaxel)))).

In some instances, the treatment extends EFS of the subject, e.g., as compared to treatment with the PD-1 axis binding antagonist without the anti-TIGIT antagonist antibody or as compared to treatment with the anti-TIGIT antagonist antibody without the PD-1 axis binding antagonist. In some instances, the treatment extends EFS of the subject, e.g., as compared to treatment with the PD-1 axis binding antagonist and the anti-TIGIT antagonist antibody without chemotherapy (e.g., a platinum-based doublet chemotherapy (e.g., a platinum-based chemotherapeutic agent (e.g., carboplatin or cisplatin) and a non-platinum-based chemotherapeutic agent (e.g., an antimetabolite (e.g., pemetrexed or gemcitabine) or a taxane (e.g., paclitaxel or nab-paclitaxel)))).

In some embodiments, a treatment described increases the MPR rate by at least 1% (e.g., by 5%, 10%, 15%, 20%, 25%, 30%, 35%, 40%, 45%, 50%, 55%, 60%, 65%, 70%, 75%, 80%, 85%, 90%, 95%, or 100%).

In some embodiments, a treatment described herein extends the pCR of the subject by at least about 2 months (e.g., by 2-120 months, by 2.5-100 months, by 3.0-80 months, by 4.0-60 months, by 5.0-48 months, by 6.0-36 months, by 8.0-24 months, or by 10-12 months, e.g., by at least about 2.4 months, 2.5 months, 2.6 months, 2.7 months, 2.8 months, 2.9 months, 3.0 months, 3.1 months, 3.2 months, 3.3 months, 3.4 months, 3.5 months, 3.6 months, 3.7 months, 3.8 months, 3.9 months, 4.0 months, 4.1 months, 4.2 months, 4.3 months, 4.4 months, 4.5 months, 4.6 months, 4.7 months, 4.8 months, 4.9 months, 5.0 months, 5.5 months, 6.0 months, 6.5 months, 7.0 months, 7.5 months, 8.0 months, 8.5 months, 9.0 months, 9.5 months, 10 months, 10.5 months, 11 months, 11.5 months, 12 months, 13 months, 14 months, 15 months, 16 months, 17 months, 18 months, 19 months, 20 months, 21 months, 22 months, 23 months, 24 months, 25 months, 26 months, 27 months, 28 months, 29 months, 30 months, 31 months, 32 months, 33 months, 34 months, 35 months, or 36 months). In some embodiments, the treatment extends the pCR of the subject by at least about 4 months (e.g., by 4-120 months, by 5-100 months, by 6-80 months, by 7-60 months, by 8-48 months, by 9-36 months, or by 10-24 months, e.g., by at least about 4.0 months, 4.1 months, 4.2 months, 4.3 months, 4.4 months, 4.5 months, 4.6 months, 4.7 months, 4.8 months, 4.9 months, 5.0 months, 5.5 months, 6.0 months, 6.5 months, 7.0 months, 7.5 months, 8.0 months, 8.5 months, 9.0 months, 9.5 months, 10 months, 10.5 months, 11 months, 11.5 months, 12 months, 13 months, 14 months, 15 months, 16 months, 17 months, 18 months, 19 months, 20 months, 21 months, 22 months, 23 months, 24 months, 25 months, 26 months, 27 months, 28 months, 29 months, 30 months, 31 months, 32 months, 33 months, 34 months, 35 months, or 36 months).

In some embodiments, a treatment described herein extends the EFS of the subject by at least about 2 months (e.g., by 2-120 months, by 2.5-100 months, by 3.0-80 months, by 4.0-60 months, by 5.0-48 months, by 6.0-36 months, by 8.0-24 months, or by 10-12 months, e.g., by at least about 2.4 months, 2.5 months, 2.6 months, 2.7 months, 2.8 months, 2.9 months, 3.0 months, 3.1 months, 3.2 months, 3.3 months, 3.4 months, 3.5 months, 3.6 months, 3.7 months, 3.8 months, 3.9 months, 4.0 months, 4.1 months, 4.2 months, 4.3 months, 4.4 months, 4.5 months, 4.6 months, 4.7 months, 4.8 months, 4.9 months, 5.0 months, 5.5 months, 6.0 months, 6.5 months, 7.0 months, 7.5 months, 8.0 months, 8.5 months, 9.0 months, 9.5 months, 10 months, 10.5 months, 11 months, 11.5 months, 12 months, 13 months, 14 months, 15 months, 16 months, 17 months, 18 months, 19 months, 20 months, 21 months, 22 months, 23 months, 24 months, 25 months, 26 months, 27 months, 28 months, 29 months, 30 months, 31 months, 32 months, 33 months, 34 months, 35 months, or 36 months). In some embodiments, the treatment extends the EFS of the subject by at least about 4 months (e.g., by 4-120 months, by 5-100 months, by 6-80 months, by 7-60 months, by 8-48 months, by 9-36 months, or by 10-24 months, e.g., by at least about 4.0 months, 4.1 months, 4.2 months, 4.3 months, 4.4 months, 4.5 months, 4.6 months, 4.7 months, 4.8 months, 4.9 months, 5.0 months, 5.5 months, 6.0 months, 6.5 months, 7.0 months, 7.5 months, 8.0 months, 8.5 months, 9.0 months, 9.5 months, 10 months, 10.5 months, 11 months, 11.5 months, 12 months, 13 months, 14 months, 15 months, 16 months, 17 months, 18 months, 19 months, 20 months, 21 months, 22 months, 23 months, 24 months, 25 months, 26 months, 27 months, 28 months, 29 months, 30 months, 31 months, 32 months, 33 months, 34 months, 35 months, or 36 months).

In some embodiments, OS is measured as the period of time from the start of treatment to death. In some instances, the treatment extends the OS of the subject by at least about 2 months (e.g., by 2-120 months, by 3-110 months, by 4-100 months, by 5-80 months, by 6-60 months, by 7-48 months, by 8-36 months, or by 10-24 months, e.g., by at least about 2 months, 2.1 months, 2.2 months, 2.3 months, 2.4 months, 2.5 months, 2.6 months, 2.7 months, 2.8 months, 2.9 months, 3.0 months, 3.1 months, 3.2 months, 3.3 months, 3.4 months, 3.5 months, 3.6 months, 3.7 months, 3.8 months, 3.9 months, 4.0 months, 4.1 months, 4.2 months, 4.3 months, 4.4 months, 4.5 months, 4.6 months, 4.7 months, 4.8 months, 4.9 months, 5.0 months, 5.5 months, 6.0 months, 6.5 months, 7.0 months, 7.5 months, 8.0 months, 8.5 months, 9.0 months, 9.5 months, 10 months, 10.5 months, 11 months, 11.5 months, 12 months, 13 months, 14 months, 15 months, 16 months, 17 months, 18 months, 19 months, 20 months, 21 months, 22 months, 23 months, 24 months, 25 months, 26 months, 27 months, 28 months, 29 months, 30 months, 31 months, 32 months, 33 months, 34 months, 35 months, or 36 months). In some instances, the treatment extends the OS of the subject by at least about 3.3 months (e.g., by 3.3-120 months, by 4-100 months, by 5-80 months, by 6-60 months, by 7-48 months, by 8-36 months, or by 10-24 months, e.g., by at least about 3.3 months, 3.4 months, 3.5 months, 3.6 months, 3.7 months, 3.8 months, 3.9 months, 4.0 months, 4.1 months, 4.2 months, 4.3 months, 4.4 months, 4.5 months, 4.6 months, 4.7 months, 4.8 months, 4.9 months, 5.0 months, 5.5 months, 6.0 months, 6.5 months, 7.0 months, 7.5 months, 8.0 months, 8.5 months, 9.0 months, 9.5 months, 10 months, 10.5 months, 11 months, 11.5 months, 12 months, 13 months, 14 months, 15 months, 16 months, 17 months, 18 months, 19 months, 20 months, 21 months, 22 months, 23 months, 24 months, 25 months, 26 months, 27 months, 28 months, 29 months, 30 months, 31 months, 32 months, 33 months, 34 months, 35 months, or 36 months). In some instances, the treatment extends the OS of the subject by at least about 5.3 months (e.g., by 5.3-120, by 6-60 months, by 7-48 months, by 8-36 months, or by 10-24 months, e.g., by at least about 5.3 months, 5.5 months, 6.0 months, 6.5 months, 7.0 months, 7.5 months, 8.0 months, 8.5 months, 9.0 months, 9.5 months, 10 months, 10.5 months, 11 months, 11.5 months, 12 months, 13 months, 14 months, 15 months, 16 months, 17 months, 18 months, 19 months, 20 months, 21 months, 22 months, 23 months, 24 months, 25 months, 26 months, 27 months, 28 months, 29 months, 30 months, 31 months, 32 months, 33 months, 34 months, 35 months, or 36 months).

C. Therapeutic and Diagnostic Methods and Uses Relating to Cervical Cancer

Cervical Cancer

Cervical cancer is the fourth most frequently diagnosed cancer and the fourth leading cause of cancer-related death. More than 500,000 women are diagnosed with cervical cancer annually worldwide, resulting in more than 300,000 deaths. Almost 90% of cervical cancer deaths occur in developing countries. In the United States, there are 13,000 new cases of invasive cervical cancer and approximately 4000 cancer-related deaths each year.

Treatment for early and locally advanced cervical cancer consists of surgery and definitive chemoradiotherapy, respectively, and can be quite effective in eliciting a remission. However, if cancer recurs or fails to resolve with primary treatment, prognosis is quite poor with 5-year survival rates of approximately 15%, which is comparable to that of patients with de novo metastatic disease. With few exceptions, the standard of care for recurrent, persistent, or de novo metastatic disease is chemotherapy plus bevacizumab based on the Gynecology Oncology Group 240 trial, which showed that bevacizumab added to chemotherapy improved median OS compared with chemotherapy alone (17 vs. 13.3 months, respectively).

Currently, no globally-accepted standard of care exists after recurrence or progression on chemotherapy plus bevacizumab. As such, treatment options for these patients largely comprise various cytotoxic chemotherapy agents, administered as either a single agent or in combination. However, given the historically low response rates of approximately 10%-15%, increasing focus has been given to whether cytotoxic chemotherapies represent an acceptable standard of care over best supportive care given the impact and burden such agents can impart on patient quality of life.

Historically low efficacy rates of existing therapies, coupled with the engagement of the immune response owing to HPV infection of the cervical epithelial cells, makes cervical cancer a particularly attractive opportunity for novel immunotherapy-based approaches.

Thus, there is an unmet need in the field for the development of efficacious immunotherapies and methods of dosing the same for the treatment of cancers (e.g., cervical cancer, e.g., Stage IVB, metastatic, recurrent, or persistent cervical cancer, e.g., a metastatic and/or recurrent PD-L1-positive cervical carcinoma).

Methods and Uses for Treating Cervical Cancer

Provided herein are methods and uses for treating cancer with a detectable expression level of PD-L1 (e.g., cervical cancer, e.g., Stage IVB, metastatic, recurrent, or persistent cervical cancer, e.g., a metastatic and/or recurrent PD-L1-positive cervical carcinoma) in a subject or population of subjects comprising administering to the subject or population of subjects one or more dosing cycles of an effective amount of an anti-TIGIT antagonist antibody and a PD-1 axis binding antagonist (e.g., an anti-PD-L1 antagonist antibody (e.g., atezolizumab) or an anti-PD-1 antagonist antibody (e.g., MDX-1106 (nivolumab) or MK-3475 (pembrolizumab, previously known as lambrolizumab))).

Dosing Regimens and Administration

The therapeutic methods and uses of the invention described herein include, in one aspect, administering to a subject or population of subjects having a cancer with a detectable expression level of PD-L1 (e.g., cervical cancer, e.g., Stage IVB, metastatic, recurrent, or persistent cervical cancer, e.g., a metastatic and/or recurrent PD-L1-positive cervical carcinoma) one or more dosing cycles of an effective amount of an anti-TIGIT antagonist antibody (e.g., an anti-TIGIT antagonist antibody as disclosed herein, e.g., tiragolumab) and an effective amount of a PD-1 axis binding antagonist (e.g., an anti-PD-L1 antagonist antibody (e.g., atezolizumab) or an anti-PD-1 antagonist antibody (e.g., MDX-1106 (nivolumab) or MK-3475 (pembrolizumab, previously known as lambrolizumab))), thereby treating the subject or population of subjects.

In some instances, the effective amount of the anti-TIGIT antagonist antibody (e.g., an anti-TIGIT antagonist antibody as disclosed herein, e.g., tiragolumab) is a dose (e.g., a fixed dose) of between about 30 mg to about 1200 mg (e.g., between about 30 mg to about 1100 mg, e.g., between about 60 mg to about 1000 mg, e.g., between about 100 mg to about 900 mg, e.g., between about 200 mg to about 800 mg, e.g., between about 300 mg to about 800 mg, e.g., between about 400 mg to about 800 mg, e.g., between about 400 mg to about 750 mg, e.g., between about 450 mg to about 750 mg, e.g., between about 500 mg to about 700 mg, e.g., between about 550 mg to about 650 mg, e.g., 600 mg±10 mg, e.g., 600±6 mg, e.g., 600±5 mg, e.g., 600±3 mg, e.g., 600±1 mg, e.g., 600±0.5 mg, e.g., 600 mg) every three weeks. In some instances, the effective amount of the anti-TIGIT antagonist antibody (e.g., an anti-TIGIT antagonist antibody as disclosed herein, e.g., tiragolumab) is a dose (e.g., a fixed dose) of between about 30 mg to about 600 mg (e.g., between about 50 mg to between 600 mg, e.g., between about 60 mg to about 600 mg, e.g., between about 100 mg to about 600 mg, e.g., between about 200 mg to about 600 mg, e.g., between about 200 mg to about 550 mg, e.g., between about 250 mg to about 500 mg, e.g., between about 300 mg to about 450 mg, e.g., between about 350 mg to about 400 mg, e.g., about 375 mg) every three weeks. In some instances, the effective amount of the anti-TIGIT antagonist antibody (e.g., an anti-TIGIT antagonist antibody as disclosed herein, e.g., tiragolumab) is a dose (e.g., a fixed dose) of about 600 mg every three weeks. In some instances, the effective amount of the anti-TIGIT antagonist antibody (e.g., an anti-TIGIT antagonist antibody as disclosed herein, e.g., tiragolumab) is a dose (e.g., a fixed dose) of between 30 mg to 1200 mg (e.g., between 30 mg to 1100 mg, e.g., between 60 mg to 1000 mg, e.g., between 100 mg to 900 mg, e.g., between 200 mg to 800 mg, e.g., between 300 mg to 800 mg, e.g., between 400 mg to 800 mg, e.g., between 400 mg to 750 mg, e.g., between 450 mg to 750 mg, e.g., between 500 mg to 700 mg, e.g., between 550 mg to 650 mg, e.g., 600 mg±10 mg, e.g., 600±6 mg, e.g., 600±5 mg, e.g., 600±3 mg, e.g., 600±1 mg, e.g., 600±0.5 mg, e.g., 600 mg) every three weeks. In some instances, the effective amount of the anti-TIGIT antagonist antibody (e.g., an anti-TIGIT antagonist antibody as disclosed herein, e.g., tiragolumab) is a dose (e.g., a fixed dose) of between 30 mg to 600 mg (e.g., between 50 mg to between 600 mg, e.g., between 60 mg to 600 mg, e.g., between 100 mg to 600 mg, e.g., between 200 mg to 600 mg, e.g., between 200 mg to 550 mg, e.g., between 250 mg to 500 mg, e.g., between 300 mg to 450 mg, e.g., between 350 mg to 400 mg, e.g., 375 mg) every three weeks. In some instances, the effective amount of the anti-TIGIT antagonist antibody (e.g., an anti-TIGIT antagonist antibody as disclosed herein, e.g., tiragolumab) is a dose (e.g., a fixed dose) of 600 mg every three weeks. In some instances, effective amount of the anti-TIGIT antagonist antibody (e.g., an anti-TIGIT antagonist antibody as disclosed herein, e.g., tiragolumab) is a dose (e.g., a fixed dose) of 600 mg every three weeks. In some instances, the dose of the anti-TIGIT antagonist antibody (e.g., an anti-TIGIT antagonist antibody as disclosed herein, e.g., tiragolumab) administered in a combination therapy (e.g., a combination treatment with a PD-1 axis binding antagonist (e.g., an anti-PD-L1 antagonist antibody (e.g., atezolizumab) or an anti-PD-1 antagonist antibody (e.g., MDX-1106 (nivolumab) or MK-3475 (pembrolizumab, previously known as lambrolizumab))) may be reduced as compared to a standard dose of the anti-TIGIT antagonist antibody administered as a monotherapy.

In some instances, the effective amount of the PD-1 axis binding antagonist (e.g., anti-PD-L1 antagonist antibody (e.g., atezolizumab)) is a dose (e.g., a fixed dose) of between about 80 mg to about 2000 mg (e.g., between about 80 mg to about 1950 mg, e.g., between about 80 mg to about 1900 mg, e.g., between about 80 mg to about 1800 mg, e.g., between about 100 mg to about 1700 mg, e.g., between about 200 mg to about 1600 mg, e.g., between about 300 mg to about 1400 mg, e.g., between about 400 mg to about 1300 mg, e.g., between about 500 mg to about 1200 mg, e.g., between about 600 mg to about 1100 mg, e.g., between about 700 mg to about 1000 mg, e.g., between about 740 mg to about 940 mg, e.g., between about 790 mg to about 890 mg, e.g., between about 815 mg to about 865 mg, e.g., between about 830 mg to about 850 mg, e.g., 840 mg±5 mg, e.g., 840±2.5 mg, e.g., 840±1.0 mg, e.g., 840±0.5 mg, e.g., 840 mg) every two weeks. In some instances, the effective amount of the PD-1 axis binding antagonist (e.g., anti-PD-L1 antagonist antibody (e.g., atezolizumab)) is a dose of between about 80 mg to about 2000 mg (e.g., between about 100 mg to about 2000 mg, e.g., between about 200 mg to about 1900 mg, e.g., between about 300 mg to about 1700 mg, e.g., between about 400 mg to about 1600 mg, e.g., between about 500 mg to about 1600 mg, e.g., between about 600 mg to about 1600 mg, e.g., between about 700 mg to about 1600 mg, e.g., between about 800 mg to about 1600 mg, e.g., between about 900 mg to about 1500 mg, e.g., between about 1000 mg to about 1400 mg, e.g., between about 1050 mg to about 1350 mg, e.g., between about 1100 mg to about 1300 mg, e.g., between about 1150 mg to about 1250 mg, e.g., between about 1175 mg to about 1225 mg, e.g., between about 1190 mg to about 1210 mg, e.g., 1200 mg±5 mg, e.g., 1200±2.5 mg, e.g., 1200±1.0 mg, e.g., 1200±0.5 mg, e.g., 1200 mg) every three weeks. In some instances, the effective amount of the PD-1 axis binding antagonist (e.g., anti-PD-L1 antagonist antibody (e.g., atezolizumab)) is a dose of between about 80 mg to about 2000 mg (e.g., between about 100 mg to about 2000 mg, e.g., between about 200 mg to about 2000 mg, e.g., between about 300 mg to about 2000 mg, e.g., between about 400 mg to about 2000 mg, e.g., between about 500 mg to about 2000 mg, e.g., between about 600 mg to about 1900 mg, e.g., between about 700 mg to about 1800 mg, e.g., between about 800 mg to about 1800 mg, e.g., between about 900 mg to about 1800 mg, e.g., between about 1000 mg to about 1800 mg, e.g., between about 1100 mg to about 1800 mg, e.g., between about 1200 mg to about 1800 mg, e.g., between about 1300 mg to about 1800 mg, e.g., between about 1400 mg to about 1800 mg, e.g., between about 1500 mg to about 1800 mg, e.g., between about 1580 mg to about 1780 mg, e.g., between about 1630 mg to about 1730 mg, e.g., between about 1655 mg to about 1705 mg, e.g., between about 1670 mg to about 1690 mg, e.g., 1680 mg±5 mg, e.g., 1680±2.5 mg, e.g., 1680±1.0 mg, e.g., 1680±0.5 mg, e.g., 1680 mg) every four weeks. In some instances, the effective amount of the PD-1 axis binding antagonist (e.g., anti-PD-L1 antagonist antibody (e.g., atezolizumab)) is a dose of about 840 mg every two weeks. In some instances, the effective amount of the PD-1 axis binding antagonist (e.g., anti-PD-L1 antagonist antibody (e.g., atezolizumab)) is a dose of 840 mg every two weeks. In some instances, the effective amount of the PD-1 axis binding antagonist (e.g., anti-PD-L1 antagonist antibody (e.g., atezolizumab)) is a dose of about 1200 mg every three weeks. In some instances, the effective amount of the PD-1 axis binding antagonist (e.g., anti-PD-L1 antagonist antibody (e.g., atezolizumab)) is a dose (e.g., a fixed dose) of between 80 mg to 2000 mg (e.g., between 80 mg to 1950 mg, e.g., between 80 mg to 1900 mg, e.g., between 80 mg to 1800 mg, e.g., between 100 mg to 1700 mg, e.g., between 200 mg to 1600 mg, e.g., between 300 mg to 1400 mg, e.g., between 400 mg to 1300 mg, e.g., between 500 mg to 1200 mg, e.g., between 600 mg to 1100 mg, e.g., between 700 mg to 1000 mg, e.g., between 740 mg to 940 mg, e.g., between 790 mg to 890 mg, e.g., between 815 mg to 865 mg, e.g., between 830 mg to 850 mg, e.g., 840 mg±5 mg, e.g., 840±2.5 mg, e.g., 840±1.0 mg, e.g., 840±0.5 mg, e.g., 840 mg) every two weeks. In some instances, the effective amount of the PD-1 axis binding antagonist (e.g., anti-PD-L1 antagonist antibody (e.g., atezolizumab)) is a dose of between 80 mg to 2000 mg (e.g., between 100 mg to 2000 mg, e.g., between 200 mg to 1900 mg, e.g., between 300 mg to 1700 mg, e.g., between 400 mg to 1600 mg, e.g., between 500 mg to 1600 mg, e.g., between 600 mg to 1600 mg, e.g., between 700 mg to 1600 mg, e.g., between 800 mg to 1600 mg, e.g., between 900 mg to 1500 mg, e.g., between 1000 mg to 1400 mg, e.g., between 1050 mg to 1350 mg, e.g., between 1100 mg to 1300 mg, e.g., between 1150 mg to 1250 mg, e.g., between 1175 mg to 1225 mg, e.g., between 1190 mg to 1210 mg, e.g., 1200 mg±5 mg, e.g., 1200±2.5 mg, e.g., 1200±1.0 mg, e.g., 1200±0.5 mg, e.g., 1200 mg) every three weeks. In some instances, the effective amount of the PD-1 axis binding antagonist (e.g., anti-PD-L1 antagonist antibody (e.g., atezolizumab)) is a dose of between 80 mg to 2000 mg (e.g., between 100 mg to 2000 mg, e.g., between 200 mg to 2000 mg, e.g., between 300 mg to 2000 mg, e.g., between 400 mg to 2000 mg, e.g., between 500 mg to 2000 mg, e.g., between 600 mg to 1900 mg, e.g., between 700 mg to 1800 mg, e.g., between 800 mg to 1800 mg, e.g., between 900 mg to 1800 mg, e.g., between 1000 mg to 1800 mg, e.g., between 1100 mg to 1800 mg, e.g., between 1200 mg to 1800 mg, e.g., between 1300 mg to 1800 mg, e.g., between 1400 mg to 1800 mg, e.g., between 1500 mg to 1800 mg, e.g., between 1580 mg to 1780 mg, e.g., between 1630 mg to 1730 mg, e.g., between 1655 mg to 1705 mg, e.g., between 1670 mg to 1690 mg, e.g., 1680 mg±5 mg, e.g., 1680±2.5 mg, e.g., 1680±1.0 mg, e.g., 1680±0.5 mg, e.g., 1680 mg) every four weeks. In some instances, the effective amount of the PD-1 axis binding antagonist (e.g., anti-PD-L1 antagonist antibody (e.g., atezolizumab)) is a dose of 840 mg every two weeks. In some instances, the effective amount of the PD-1 axis binding antagonist (e.g., anti-PD-L1 antagonist antibody (e.g., atezolizumab)) is a dose of 840 mg every two weeks. In some instances, the effective amount of the PD-1 axis binding antagonist (e.g., anti-PD-L1 antagonist antibody (e.g., atezolizumab)) is a dose of 1200 mg every three weeks. In some instances, the effective amount of the PD-1 axis binding antagonist (e.g., anti-PD-L1 antagonist antibody (e.g., atezolizumab)) is a dose of 1200 mg every three weeks. In some instances, the effective amount of the PD-1 axis binding antagonist (e.g., anti-PD-L1 antagonist antibody (e.g., atezolizumab)) is a dose of about 1680 mg every four weeks. In some instances, the effective amount of the PD-1 axis binding antagonist (e.g., anti-PD-L1 antagonist antibody (e.g., atezolizumab)) is a dose of 1680 mg every four weeks. In some instances, the dose of the PD-1 axis binding antagonist (e.g., anti-PD-L1 antagonist antibody (e.g., atezolizumab)) administered in a combination therapy (e.g., a combination treatment with an anti-TIGIT antagonist antibody, such as an anti-TIGIT antagonist antibody disclosed herein, e.g., tiragolumab) may be reduced as compared to a standard dose of the anti-PD-L1 antagonist antibody administered as a monotherapy.

In some instances, the effective amount of the PD-1 axis binding antagonist (e.g., anti-PD-L1 antagonist antibody (e.g., atezolizumab)) is a dose of between about 0.01 mg/kg to about 50 mg/kg of the subject's body weight (e.g., between about 0.01 mg/kg to about 45 mg/kg, e.g., between about 0.1 mg/kg to about 40 mg/kg, e.g., between about 1 mg/kg to about 35 mg/kg, e.g., between about 2.5 mg/kg to about 30 mg/kg, e.g., between about 5 mg/kg to about 25 mg/kg, e.g., between about 10 mg/kg to about 20 mg/kg, e.g., between about 12.5 mg/kg to about 15 mg/kg, e.g., about 15±2 mg/kg, about 15±1 mg/kg, about 15±0.5 mg/kg, about 15±0.2 mg/kg, or about 15±0.1 mg/kg, e.g., about 15 mg/kg) every three weeks. In some instances, the effective amount of the PD-1 axis binding antagonist (e.g., anti-PD-L1 antagonist antibody (e.g., atezolizumab)) is a dose of between about 0.01 mg/kg to about 15 mg/kg of the subject's body weight (e.g., between about 0.1 mg/kg to about 15 mg/kg, e.g., between about 0.5 mg/kg to about 15 mg/kg, e.g., between about 1 mg/kg to about 15 mg/kg, e.g., between about 2.5 mg/kg to about 15 mg/kg, e.g., between about 5 mg/kg to about 15 mg/kg, e.g., between about 7.5 mg/kg to about 15 mg/kg, e.g., between about 10 mg/kg to about 15 mg/kg, e.g., between about 12.5 mg/kg to about 15 mg/kg, e.g., between about 14 mg/kg to about 15 mg/kg, e.g., about 15±1 mg/kg, e.g., about 15±0.5 mg/kg, e.g., about 15±0.2 mg/kg, e.g., about 15±0.1 mg/kg, e.g., about 15 mg/kg) every three weeks. In some instances, the effective amount of anti-PD-L1 antagonist antibody (e.g., atezolizumab) is a dose of about 15 mg/kg administered every three weeks. In some instances, the effective amount of the PD-1 axis binding antagonist (e.g., anti-PD-L1 antagonist antibody (e.g., atezolizumab)) is a dose of between 0.01 mg/kg to 50 mg/kg of the subject's body weight (e.g., between 0.01 mg/kg to 45 mg/kg, e.g., between 0.1 mg/kg to 40 mg/kg, e.g., between 1 mg/kg to 35 mg/kg, e.g., between 2.5 mg/kg to 30 mg/kg, e.g., between 5 mg/kg to 25 mg/kg, e.g., between 10 mg/kg to 20 mg/kg, e.g., between 12.5 mg/kg to 15 mg/kg, e.g., 15±2 mg/kg, 15±1 mg/kg, 15±0.5 mg/kg, 15±0.2 mg/kg, or 15±0.1 mg/kg, e.g., 15 mg/kg) every three weeks. In some instances, the effective amount of the PD-1 axis binding antagonist (e.g., anti-PD-L1 antagonist antibody (e.g., atezolizumab)) is a dose of between 0.01 mg/kg to 15 mg/kg of the subject's body weight (e.g., between 0.1 mg/kg to 15 mg/kg, e.g., between 0.5 mg/kg to 15 mg/kg, e.g., between 1 mg/kg to 15 mg/kg, e.g., between 2.5 mg/kg to 15 mg/kg, e.g., between 5 mg/kg to 15 mg/kg, e.g., between 7.5 mg/kg to 15 mg/kg, e.g., between 10 mg/kg to 15 mg/kg, e.g., between 12.5 mg/kg to 15 mg/kg, e.g., between 14 mg/kg to 15 mg/kg, e.g., 15±1 mg/kg, e.g., 15±0.5 mg/kg, e.g., 15±0.2 mg/kg, e.g., 15±0.1 mg/kg, e.g., 15 mg/kg) every three weeks. In some instances, the effective amount of anti-PD-L1 antagonist antibody (e.g., atezolizumab) is a dose of 15 mg/kg administered every three weeks. In some instances, the dose of the PD-1 axis binding antagonist (e.g., anti-PD-L1 antagonist antibody (e.g., atezolizumab)) administered in a combination therapy (e.g., a combination treatment with an anti-TIGIT antagonist antibody, such as an anti-TIGIT antagonist antibody disclosed herein, e.g., tiragolumab) may be reduced as compared to a standard dose of the PD-1 axis binding antagonist (e.g., anti-PD-L1 antagonist antibody (e.g., atezolizumab) or anti-PD-1 antagonist antibody (e.g., MDX-1106 (nivolumab) or MK-3475 (pembrolizumab, previously known as lambrolizumab))) administered as a monotherapy.

In some instances, the effective amount of the PD-1 axis binding antagonist (e.g., anti-PD-1 antagonist antibody (e.g., pembrolizumab)) is a dose (e.g., a fixed dose) of between about 20 mg to about 1000 mg (e.g., between about 40 mg to about 900 mg, e.g., between about 60 mg to about 800 mg, e.g., between about 80 mg to about 700 mg, e.g., between about 80 mg to about 600 mg, e.g., between about 100 mg to about 500 mg, e.g., between about 120 mg to about 400 mg, e.g., between about 140 mg to about 300 mg, e.g., between about 160 mg to about 350 mg, e.g., between about 180 mg to about 300 mg, e.g., between about 180 mg to about 250 mg, e.g., between about 180 mg to about 220 mg, e.g., between about 190 mg to about 210 mg, e.g., 200 mg±5 mg, e.g., 200±2.5 mg, e.g., 200±1.0 mg, e.g., 200±0.5 mg, e.g., 200 mg) every three weeks. In some instances, the effective amount of the PD-1 axis binding antagonist (e.g., anti-PD-1 antagonist antibody (e.g., pembrolizumab)) is a dose (e.g., a fixed dose) of between 20 mg to 1000 mg (e.g., between 40 mg to 900 mg, e.g., between 60 mg to 800 mg, e.g., between 80 mg to 700 mg, e.g., between 80 mg to 600 mg, e.g., between 100 mg to 500 mg, e.g., between 120 mg to 400 mg, e.g., between 140 mg to 300 mg, e.g., between 160 mg to 350 mg, e.g., between 180 mg to 300 mg, e.g., between 180 mg to 250 mg, e.g., between 180 mg to 220 mg, e.g., between 190 mg to 210 mg, e.g., 200 mg±5 mg, e.g., 200±2.5 mg, e.g., 200±1.0 mg, e.g., 200±0.5 mg, e.g., 200 mg) every three weeks. In some instances, the effective amount of the PD-1 axis binding antagonist (e.g., anti-PD-1 antagonist antibody (e.g., pembrolizumab)) is a dose (e.g., a fixed dose) of 200 mg every three weeks. In some instances, the dose of the PD-1 axis binding antagonist (e.g., anti-PD-1 antagonist antibody (e.g., pembrolizumab)) administered in a combination therapy (e.g., a combination treatment with an anti-TIGIT antagonist antibody, such as an anti-TIGIT antagonist antibody disclosed herein, e.g., tiragolumab) may be reduced as compared to a standard dose of the anti-PD-L1 antagonist antibody administered as a monotherapy.

In some instances, the effective amount of the PD-1 axis binding antagonist (e.g., anti-PD-1 antagonist antibody (e.g., nivolumab)) is a dose (e.g., a fixed dose) of between about 20 mg to about 1000 mg (e.g., between about 40 mg to about 900 mg, e.g., between about 60 mg to about 800 mg, e.g., between about 80 mg to about 700 mg, e.g., between about 80 mg to about 600 mg, e.g., between about 100 mg to about 500 mg, e.g., between about 120 mg to about 400 mg, e.g., between about 140 mg to about 300 mg, e.g., between about 160 mg to about 350 mg, e.g., between about 180 mg to about 300 mg, e.g., between about 200 mg to about 280 mg, e.g., between about 220 mg to about 260 mg, e.g., between about 230 mg to about 250 mg, e.g., 240 mg±5 mg, e.g., 240±2.5 mg, e.g., 240±1.0 mg, e.g., 240±0.5 mg, e.g., 240 mg) every two weeks. In some instances, the effective amount of the PD-1 axis binding antagonist (e.g., anti-PD-1 antagonist antibody (e.g., nivolumab)) is a dose of 240 mg every two weeks. In some instances, the effective amount of the PD-1 axis binding antagonist (e.g., anti-PD-1 antagonist antibody (e.g., nivolumab)) is a dose of between about 100 mg to about 1000 mg (e.g., between about 200 mg to about 900 mg, e.g., between about 300 mg to about 800 mg, e.g., between about 400 mg to about 700 mg, e.g., between about 400 mg to about 600 mg, e.g., between about 400 mg to about 550 mg, e.g., between about 420 mg to about 540 mg, e.g., between about 440 mg to about 520 mg, e.g., between about 460 mg to about 500 mg, e.g., between about 470 mg to about 490 mg, e.g., 480 mg±5 mg, e.g., 480±2.5 mg, e.g., 480±1.0 mg, e.g., 480±0.5 mg, e.g., 480 mg) every four weeks. In some instances, the effective amount of the PD-1 axis binding antagonist (e.g., anti-PD-1 antagonist antibody (e.g., nivolumab)) is a dose (e.g., a fixed dose) of between 20 mg to 1000 mg (e.g., between 40 mg to 900 mg, e.g., between 60 mg to 800 mg, e.g., between 80 mg to 700 mg, e.g., between 80 mg to 600 mg, e.g., between 100 mg to 500 mg, e.g., between 120 mg to 400 mg, e.g., between 140 mg to 300 mg, e.g., between 160 mg to 350 mg, e.g., between 180 mg to 300 mg, e.g., between 200 mg to 280 mg, e.g., between 220 mg to 260 mg, e.g., between 230 mg to 250 mg, e.g., 240 mg±5 mg, e.g., 240±2.5 mg, e.g., 240±1.0 mg, e.g., 240±0.5 mg, e.g., 240 mg) every two weeks. In some instances, the effective amount of the PD-1 axis binding antagonist (e.g., anti-PD-1 antagonist antibody (e.g., nivolumab)) is a dose of 240 mg every two weeks. In some instances, the effective amount of the PD-1 axis binding antagonist (e.g., anti-PD-1 antagonist antibody (e.g., nivolumab)) is a dose of between 100 mg to 1000 mg (e.g., between 200 mg to 900 mg, e.g., between 300 mg to 800 mg, e.g., between 400 mg to 700 mg, e.g., between 400 mg to 600 mg, e.g., between 400 mg to 550 mg, e.g., between 420 mg to 540 mg, e.g., between 440 mg to 520 mg, e.g., between 460 mg to 500 mg, e.g., between 470 mg to 490 mg, e.g., 480 mg±5 mg, e.g., 480±2.5 mg, e.g., 480±1.0 mg, e.g., 480±0.5 mg, e.g., 480 mg) every four weeks. In some instances, the effective amount of the PD-1 axis binding antagonist (e.g., anti-PD-1 antagonist antibody (e.g., nivolumab)) is a dose of 480 mg every four weeks. In some instances, the dose of the PD-1 axis binding antagonist (e.g., anti-PD-1 antagonist antibody (e.g., nivolumab)) administered in a combination therapy (e.g., a combination treatment with an anti-TIGIT antagonist antibody, such as an anti-TIGIT antagonist antibody disclosed herein, e.g., tiragolumab) may be reduced as compared to a standard dose of the anti-PD-L1 antagonist antibody administered as a monotherapy.

In any of the methods and uses of the invention, the anti-TIGIT antagonist antibody (e.g., an anti-TIGIT antagonist antibody as disclosed herein, e.g., tiragolumab) and the PD-1 axis binding antagonist (e.g., anti-PD-L1 antagonist antibody (e.g., atezolizumab) or anti-PD-1 antagonist antibody (e.g., MDX-1106 (nivolumab) or MK-3475 (pembrolizumab, previously known as lambrolizumab))) may be administered in one or more dosing cycles (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 or more dosing cycles). In some instances, the dosing cycles of the anti-TIGIT antagonist antibody (e.g., an anti-TIGIT antagonist antibody as disclosed herein, e.g., tiragolumab) and the PD-1 axis binding antagonist (e.g., anti-PD-L1 antagonist antibody (e.g., atezolizumab) or anti-PD-1 antagonist antibody (e.g., MDX-1106 (nivolumab) or MK-3475 (pembrolizumab, previously known as lambrolizumab))) continue until there is a loss of clinical benefit (e.g., confirmed disease progression, drug resistance, death, or unacceptable toxicity). In some instances, the length of each dosing cycle is about 14 to 28 days (e.g., 14 days, 15 days, 16 days, 17 days, 18 days, 19 days, 20 days, 21 days, 22 days, 23 days, 24 days, 25 days, 26 days, 27 days, or 28 days). In some instances, the length of each dosing cycle is about 21 days. In some instances, the length of each dosing cycle is about 14 days. In some instances, the length of each dosing cycle is about 28 days. In some instances, the anti-TIGIT antagonist antibody (e.g., an anti-TIGIT antagonist antibody as disclosed herein, e.g., tiragolumab) is administered on about Day 1 (e.g., Day 1±3 days) of each dosing cycle. For example, the anti-TIGIT antagonist antibody (e.g., an anti-TIGIT antagonist antibody as disclosed herein, e.g., tiragolumab) is administered intravenously at a dose (e.g., a fixed dose) of about 600 mg on Day 1 of each 21-day cycle (i.e., at a dose of about 600 mg every three weeks). In some instances, the anti-TIGIT antagonist antibody (e.g., an anti-TIGIT antagonist antibody as disclosed herein, e.g., tiragolumab) is administered intravenously at a dose (e.g., a fixed dose) of 600 mg on Day 1 of each 21-day cycle (i.e., at a dose of 600 mg every three weeks). Similarly, in some instances, the PD-1 axis binding antagonist (e.g., an anti-PD-L1 antagonist antibody (e.g., atezolizumab) or an anti-PD-1 antagonist antibody (e.g., MDX-1106 (nivolumab) or MK-3475 (pembrolizumab, previously known as lambrolizumab))) is administered on about Day 1 (e.g., Day 1±3 days) of each dosing cycle. For example, the PD-1 axis binding antagonist (e.g., anti-PD-L1 antagonist antibody (e.g., atezolizumab)) is administered intravenously at a dose of about 1200 mg on Day 1 of each 21-day cycle (i.e., at a dose of about 1200 mg every three weeks). For example, the PD-1 axis binding antagonist (e.g., anti-PD-1 antagonist antibody (e.g., pembrolizumab)) is administered intravenously at a dose of about 200 mg on Day 1 of each 21-day cycle (i.e., at a dose of about 200 mg every three weeks). For example, the PD-1 axis binding antagonist (e.g., anti-PD-1 antagonist antibody (e.g., nivolumab)) is administered intravenously at a dose of about 240 mg on Day 1 of each 14-day cycle (i.e., at a dose of about 240 mg every two weeks). For example, the PD-1 axis binding antagonist (e.g., anti-PD-1 antagonist antibody (e.g., nivolumab)) is administered intravenously at a dose of about 480 mg on Day 1 of each 28-day cycle (i.e., at a dose of about 480 mg every four weeks). In some instances, the PD-1 axis binding antagonist (e.g., anti-PD-L1 antagonist antibody (e.g., atezolizumab)) is administered intravenously at a dose of about 1200 mg on Day 1 of each 21-day cycle (i.e., at a dose of about 1200 mg every three weeks). For example, the PD-1 axis binding antagonist (e.g., anti-PD-1 antagonist antibody (e.g., pembrolizumab)) is administered intravenously at a dose of about 200 mg on Day 1 of each 21-day cycle (i.e., at a dose of about 200 mg every three weeks). For example, the PD-1 axis binding antagonist (e.g., anti-PD-1 antagonist antibody (e.g., nivolumab)) is administered intravenously at a dose of about 240 mg on Day 1 of each 14-day cycle (i.e., at a dose of about 240 mg every two weeks). For example, the PD-1 axis binding antagonist (e.g., anti-PD-1 antagonist antibody (e.g., nivolumab)) is administered intravenously at a dose of about 480 mg on Day 1 of each 28-day cycle (i.e., at a dose of about 480 mg every four weeks).

In some instances, both the anti-TIGIT antagonist antibody (e.g., an anti-TIGIT antagonist antibody as disclosed herein, e.g., tiragolumab) and the PD-1 axis binding antagonist (e.g., anti-PD-L1 antagonist antibody (e.g., atezolizumab) or anti-PD-1 antagonist antibody (e.g., MDX-1106 (nivolumab) or MK-3475 (pembrolizumab, previously known as lambrolizumab))) are administered on about Day 1 (e.g., Day 1±3 days) of each dosing cycle. For example, the anti-TIGIT antagonist antibody (e.g., an anti-TIGIT antagonist antibody as disclosed herein, e.g., tiragolumab) is administered intravenously at a dose (e.g., a fixed dose) of about 600 mg on Day 1 of each 21-day cycle (i.e., at a dose of about 600 mg every three weeks), and the PD-1 axis binding antagonist (e.g., anti-PD-L1 antagonist antibody (e.g., atezolizumab)) is administered intravenously at a dose (e.g., a fixed dose) of about 1200 mg on Day 1 of each 21-day cycle (i.e., at a dose of about 1200 mg every three weeks). For example, the anti-TIGIT antagonist antibody (e.g., an anti-TIGIT antagonist antibody as disclosed herein, e.g., tiragolumab) is administered intravenously at a dose of about 600 mg on Day 1 of each 21-day cycle (i.e., at a dose of about 600 mg every three weeks), and the PD-1 axis binding antagonist (e.g., anti-PD-L1 antagonist antibody (e.g., pembrolizumab)) is administered intravenously at a dose of about 200 mg on Day 1 of each 21-day cycle (i.e., at a dose of about 200 mg every three weeks). In some examples, the anti-TIGIT antagonist antibody (e.g., an anti-TIGIT antagonist antibody as disclosed herein, e.g., tiragolumab) is administered intravenously at a dose (e.g., a fixed dose) of 600 mg on Day 1 of each 21-day cycle (i.e., at a dose of 600 mg every three weeks), and the PD-1 axis binding antagonist (e.g., anti-PD-L1 antagonist antibody (e.g., atezolizumab)) is administered intravenously at a dose (e.g., a fixed dose) of 1200 mg on Day 1 of each 21-day cycle (i.e., at a dose of 1200 mg every three weeks). For example, the anti-TIGIT antagonist antibody (e.g., an anti-TIGIT antagonist antibody as disclosed herein, e.g., tiragolumab) is administered intravenously at a dose of 600 mg on Day 1 of each 21-day cycle (i.e., at a dose of 600 mg every three weeks), and the PD-1 axis binding antagonist (e.g., anti-PD-L1 antagonist antibody (e.g., pembrolizumab)) is administered intravenously at a dose of 200 mg on Day 1 of each 21-day cycle (i.e., at a dose of 200 mg every three weeks).

In some instances, both the anti-TIGIT antagonist antibody (e.g., an anti-TIGIT antagonist antibody as disclosed herein, e.g., tiragolumab) and the PD-1 axis binding antagonist (e.g., anti-PD-L1 antagonist antibody (e.g., atezolizumab) or anti-PD-1 antagonist antibody (e.g., MDX-1106 (nivolumab) or MK-3475 (pembrolizumab, previously known as lambrolizumab))) are administered on about Day 1 (e.g., Day 1±3 days) of the first dosing cycle. For example, the anti-TIGIT antagonist antibody (e.g., an anti-TIGIT antagonist antibody as disclosed herein, e.g., tiragolumab) is administered intravenously at a dose (e.g., a fixed dose) of about 600 mg on Day 1 of each 21-day cycle (i.e., at a dose of about 600 mg every three weeks), and the PD-1 axis binding antagonist (e.g., anti-PD-L1 antagonist antibody (e.g., nivolumab)) is administered intravenously at a dose (e.g., a fixed dose) of about 240 mg on Day 1 of each 14-day cycle (i.e., at a dose of about 240 mg every two weeks). For example, the anti-TIGIT antagonist antibody (e.g., an anti-TIGIT antagonist antibody as disclosed herein, e.g., tiragolumab) is administered intravenously at a dose of about 600 mg on Day 1 of each 21-day cycle (i.e., at a dose of about 600 mg every three weeks), and the PD-1 axis binding antagonist (e.g., anti-PD-L1 antagonist antibody (e.g., nivolumab)) is administered intravenously at a dose of about 480 mg on Day 1 of each 28-day cycle (i.e., at a dose of about 480 mg every four weeks). In some examples, the anti-TIGIT antagonist antibody (e.g., an anti-TIGIT antagonist antibody as disclosed herein, e.g., tiragolumab) is administered intravenously at a dose (e.g., a fixed dose) of 600 mg on Day 1 of each 21-day cycle (i.e., at a dose of 600 mg every three weeks), and the PD-1 axis binding antagonist (e.g., anti-PD-L1 antagonist antibody (e.g., nivolumab)) is administered intravenously at a dose (e.g., a fixed dose) of 240 mg on Day 1 of each 14-day cycle (i.e., at a dose of 240 mg every two weeks). For example, the anti-TIGIT antagonist antibody (e.g., an anti-TIGIT antagonist antibody as disclosed herein, e.g., tiragolumab) is administered intravenously at a dose of 600 mg on Day 1 of each 21-day cycle (i.e., at a dose of 600 mg every three weeks), and the PD-1 axis binding antagonist (e.g., anti-PD-L1 antagonist antibody (e.g., nivolumab)) is administered intravenously at a dose of 480 mg on Day 1 of each 28-day cycle (i.e., at a dose of 480 mg every four weeks).

In some instances, the anti-TIGIT antagonist antibody (e.g., an anti-TIGIT antagonist antibody as disclosed herein, e.g., tiragolumab) is administered to the subject or population of subjects by intravenous infusion over about 60±15 minutes (e.g., about 50 minutes, about 51 minutes, about 52 minutes, about 53 minutes, about 54 minutes, about 55 minutes, about 56 minutes, about 57 minutes, about 58 minutes, about 59 minutes, about 60 minutes, about 61 minutes, about 62 minutes, about 63 minutes, about 64 minutes, about 65 minutes, about 66 minutes, about 67 minutes, about 68 minutes, about 69 minutes, or about 70 minutes). In some instances, the PD-1 axis binding antagonist (e.g., anti-PD-L1 antagonist antibody (e.g., atezolizumab) or anti-PD-1 antagonist antibody (e.g., MDX-1106 (nivolumab) or MK-3475 (pembrolizumab, previously known as lambrolizumab))) is administered to the subject or population of subjects by intravenous infusion over about 60±15 minutes (e.g., about 45 minutes, about 46 minutes, about 47 minutes, about 48 minutes, about 49 minutes, about 50 minutes, about 51 minutes, about 52 minutes, about 53 minutes, about 54 minutes, about 55 minutes, about 56 minutes, about 57 minutes, about 58 minutes, about 59 minutes, about 60 minutes, about 61 minutes, about 62 minutes, about 63 minutes, about 64 minutes, about 65 minutes, about 66 minutes, about 67 minutes, about 68 minutes, about 69 minutes, about 70 minutes, about 71 minutes, about 72 minutes, about 73 minutes, about 74 minutes, or about 75 minutes).

In some instances, the anti-TIGIT antagonist antibody (e.g., an anti-TIGIT antagonist antibody as disclosed herein, e.g., tiragolumab) is administered to the subject or population of subjects by intravenous infusion over about 30±10 minutes (e.g., about 20 minutes, about 21 minutes, about 22 minutes, about 23 minutes, about 24 minutes, about 25 minutes, about 26 minutes, about 27 minutes, about 28 minutes, about 29 minutes, about 30 minutes, about 31 minutes, about 32 minutes, about 33 minutes, about 34 minutes, about 35 minutes, about 36 minutes, about 37 minutes, about 38 minutes, about 39 minutes, or about 40 minutes). In some instances, the PD-1 axis binding antagonist (e.g., anti-PD-L1 antagonist antibody (e.g., atezolizumab) or anti-PD-1 antagonist antibody (e.g., MDX-1106 (nivolumab) or MK-3475 (pembrolizumab, previously known as lambrolizumab))) is administered to the subject or population of subjects by intravenous infusion over about 30±10 minutes (e.g., about 20 minutes, about 21 minutes, about 22 minutes, about 23 minutes, about 24 minutes, about 25 minutes, about 26 minutes, about 27 minutes, about 28 minutes, about 29 minutes, about 30 minutes, about 31 minutes, about 32 minutes, about 33 minutes, about 34 minutes, about 35 minutes, about 36 minutes, about 37 minutes, about 38 minutes, about 39 minutes, or about 40 minutes).

In some instances, the anti-TIGIT antagonist antibody (e.g., an anti-TIGIT antagonist antibody as disclosed herein, e.g., tiragolumab) is administered to the subject or population of subjects before the PD-1 axis binding antagonist (e.g., anti-PD-L1 antagonist antibody (e.g., atezolizumab) or anti-PD-1 antagonist antibody (e.g., MDX-1106 (nivolumab) or MK-3475 (pembrolizumab, previously known as lambrolizumab))). In some instances, for example, following administration of the anti-TIGIT antagonist antibody and before administration of the PD-1 axis binding antagonist (e.g., anti-PD-L1 antagonist antibody (e.g., atezolizumab) or anti-PD-1 antagonist antibody (e.g., MDX-1106 (nivolumab) or MK-3475 (pembrolizumab, previously known as lambrolizumab))), the method includes an intervening first observation period. In some instances, the method further includes a second observation period following administration of the PD-1 axis binding antagonist (e.g., anti-PD-L1 antagonist antibody (e.g., atezolizumab) or anti-PD-1 antagonist antibody (e.g., MDX-1106 (nivolumab) or MK-3475 (pembrolizumab, previously known as lambrolizumab))). In some instances, the method includes both a first observation period following administration of the anti-TIGIT antagonist antibody and second observation period following administration of the PD-1 axis binding antagonist (e.g., anti-PD-L1 antagonist antibody (e.g., atezolizumab) or anti-PD-1 antagonist antibody (e.g., MDX-1106 (nivolumab) or MK-3475 (pembrolizumab, previously known as lambrolizumab))). In some instances, the first and second observation periods are each between about 30 minutes to about 60 minutes in length. In instances in which the first and second observation periods are each about 60 minutes in length, the method may include recording the subject or population of subjects' vital signs (e.g., pulse rate, respiratory rate, blood pressure, and temperature) at about 30±10 minutes after administration of the anti-TIGIT antagonist antibody and PD-1 axis binding antagonist (e.g., anti-PD-L1 antagonist antibody (e.g., atezolizumab) or anti-PD-1 antagonist antibody (e.g., MDX-1106 (nivolumab) or MK-3475 (pembrolizumab, previously known as lambrolizumab))) during the first and second observation periods, respectively. In instances in which the first and second observation periods are each about 30 minutes in length, the method may include recording the subject or population of subjects' vital signs (e.g., pulse rate, respiratory rate, blood pressure, and temperature) at about 15±10 minutes after administration of the anti-TIGIT antagonist antibody and PD-1 axis binding antagonist (e.g., anti-PD-L1 antagonist antibody (e.g., atezolizumab) or anti-PD-1 antagonist antibody (e.g., MDX-1106 (nivolumab) or MK-3475 (pembrolizumab, previously known as lambrolizumab))) during the first and second observation periods, respectively.

In other instances, the PD-1 axis binding antagonist (e.g., anti-PD-L1 antagonist antibody (e.g., atezolizumab) or anti-PD-1 antagonist antibody (e.g., MDX-1106 (nivolumab) or MK-3475 (pembrolizumab, previously known as lambrolizumab))) is administered to the subject or population of subjects before the anti-TIGIT antagonist antibody (e.g., an anti-TIGIT antagonist antibody as disclosed herein, e.g., tiragolumab). In some instances, for example, following administration of a PD-1 axis binding antagonist (e.g., an anti-PD-L1 antagonist antibody (e.g., atezolizumab) or an anti-PD-1 antagonist antibody (e.g., MDX-1106 (nivolumab) or MK-3475 (pembrolizumab, previously known as lambrolizumab))) and before administration of the anti-TIGIT antagonist antibody, the method includes an intervening first observation period. In some instances, the method includes a second observation period following administration of the anti-TIGIT antagonist antibody. In some instances, the method includes both a first observation period following administration of the PD-1 axis binding antagonist (e.g., anti-PD-L1 antagonist antibody (e.g., atezolizumab) or anti-PD-1 antagonist antibody (e.g., MDX-1106 (nivolumab) or MK-3475 (pembrolizumab, previously known as lambrolizumab))) and second observation period following administration of the anti-TIGIT antagonist antibody. In some instances, the first and second observation periods are each between about 30 minutes to about 60 minutes in length. In instances in which the first and second observation periods are each about 60 minutes in length, the method may include recording the subject or population of subjects' vital signs (e.g., pulse rate, respiratory rate, blood pressure, and temperature) at about 30±10 minutes after administration of the PD-1 axis binding antagonist (e.g., anti-PD-L1 antagonist antibody (e.g., atezolizumab) or anti-PD-1 antagonist antibody (e.g., MDX-1106 (nivolumab) or MK-3475 (pembrolizumab, previously known as lambrolizumab))) and anti-TIGIT antagonist antibody during the first and second observation periods, respectively. In instances in which the first and second observation periods are each about 30 minutes in length, the method may include recording the subject or population of subjects' vital signs (e.g., pulse rate, respiratory rate, blood pressure, and temperature) at about 15±10 minutes after administration of the PD-1 axis binding antagonist (e.g., anti-PD-L1 antagonist antibody (e.g., atezolizumab) or anti-PD-1 antagonist antibody (e.g., MDX-1106 (nivolumab) or MK-3475 (pembrolizumab, previously known as lambrolizumab))) and anti-TIGIT antagonist antibody during the first and second observation periods, respectively.

In other instances, the anti-TIGIT antagonist antibody (e.g., an anti-TIGIT antagonist antibody as disclosed herein, e.g., tiragolumab) and the PD-1 axis binding antagonist (e.g., anti-PD-L1 antagonist antibody (e.g., atezolizumab) or anti-PD-1 antagonist antibody (e.g., MDX-1106 (nivolumab) or MK-3475 (pembrolizumab, previously known as lambrolizumab))) are administered to the subject or population of subjects simultaneously. In some instances, for example, following administration of the anti-TIGIT antagonist antibody and the PD-1 axis binding antagonist (e.g., anti-PD-L1 antagonist antibody (e.g., atezolizumab) or anti-PD-1 antagonist antibody (e.g., MDX-1106 (nivolumab) or MK-3475 (pembrolizumab, previously known as lambrolizumab))) the method includes an observation period. In some instances, the observation period is between about 30 minutes to about 60 minutes in length. In instances in which the observation period is about 60 minutes in length, the method may include recording the subject or population of subjects' vital signs (e.g., pulse rate, respiratory rate, blood pressure, and temperature) at about 30±10 minutes after administration of the PD-1 axis binding antagonist (e.g., anti-PD-L1 antagonist antibody (e.g., atezolizumab) or anti-PD-1 antagonist antibody (e.g., MDX-1106 (nivolumab) or MK-3475 (pembrolizumab, previously known as lambrolizumab))) and anti-TIGIT antagonist antibody during the observation period. In instances in which the observation period is about 30 minutes in length, the method may include recording the subject or population of subjects' vital signs (e.g., pulse rate, respiratory rate, blood pressure, and temperature) at about 15±10 minutes after administration of the PD-1 axis binding antagonist (e.g., anti-PD-L1 antagonist antibody (e.g., atezolizumab) or anti-PD-1 antagonist antibody (e.g., MDX-1106 (nivolumab) or MK-3475 (pembrolizumab, previously known as lambrolizumab))) and anti-TIGIT antagonist antibody during the observation period.

In another aspect, the invention provides a method of treating a subject or population of subjects having a Stage IVB, metastatic, recurrent, or persistent cervical cancer by administering to the subject or population of subjects one or more dosing cycles of an anti-TIGIT antagonist antibody at a dose (e.g., a fixed dose) of 600 mg every three weeks and atezolizumab at a dose (e.g., a fixed dose) of 1200 mg every three weeks, wherein the anti-TIGIT antagonist antibody has a VH domain having the amino acid sequence of SEQ ID NO: 17 or 18 and a VL domain having the amino acid sequence of SEQ ID NO: 19, as described in further detail below. In some instances, the anti-TIGIT antagonist antibody has a VH domain having the amino acid sequence of SEQ ID NO: 17 and a VL domain having the amino acid sequence of SEQ ID NO: 19. In some instances, the anti-TIGIT antagonist antibody has a VH domain having the amino acid sequence of SEQ ID NO: 18 and a VL domain having the amino acid sequence of SEQ ID NO: 19.

In another aspect, the invention provides a method of treating a subject or population of subjects having a Stage IVB, metastatic, recurrent, or persistent cervical cancer by administering to the subject or population of subjects one or more dosing cycles of tiragolumab at a dose (e.g., a fixed dose) of 600 mg every three weeks and atezolizumab at a dose (e.g., a fixed dose) of 1200 mg every three weeks.

In another aspect, the invention provides a method of treating a subject or population of subjects having a Stage IVB, metastatic, recurrent, or persistent cervical cancer by administering to the subject or population of subjects one or more dosing cycles of tiragolumab at a dose (e.g., a fixed dose) of 600 mg every three weeks and pembrolizumab at a dose (e.g., a fixed dose) of 200 mg every three weeks.

In another aspect, the invention provides a method of treating a subject or population of subjects having a Stage IVB, metastatic, recurrent, or persistent cervical cancer by administering to the subject or population of subjects one or more dosing cycles of tiragolumab at a dose (e.g., a fixed dose) of 600 mg every three weeks and nivolumab at a dose (e.g., a fixed dose) of 240 mg every two weeks.

In another aspect, the invention provides a method of treating a subject or population of subjects having a Stage IVB, metastatic, recurrent, or persistent cervical cancer by administering to the subject or population of subjects one or more dosing cycles of tiragolumab at a dose (e.g., a fixed dose) of 600 mg every three weeks and nivolumab at a dose (e.g., a fixed dose) of 480 mg every four weeks.

In another aspect, the invention provides an anti-TIGIT antagonist antibody (e.g., an anti-TIGIT antagonist antibody disclosed herein, e.g., tiragolumab) and an PD-1 axis binding antagonist (e.g., an anti-PD-L1 antagonist antibody (e.g., atezolizumab) or an anti-PD-1 antagonist antibody (e.g., MDX-1106 (nivolumab) or MK-3475 (pembrolizumab, previously known as lambrolizumab))) for use in a method of treating a subject or population of subjects having a cancer with a detectable expression level of PD-L1 (e.g., cervical cancer, e.g., Stage IVB, metastatic, recurrent, or persistent cervical cancer, e.g., a metastatic and/or recurrent PD-L1-positive cervical carcinoma), wherein the method comprises administering to the subject or population of subjects one or more dosing cycles of an effective amount of an anti-TIGIT antagonist antibody and an effective amount of a PD-1 axis binding antagonist (e.g., an anti-PD-L1 antagonist antibody (e.g., atezolizumab) or an anti-PD-1 antagonist antibody (e.g., MDX-1106 (nivolumab) or MK-3475 (pembrolizumab, previously known as lambrolizumab))).

In some instances, the effective amount of the anti-TIGIT antagonist antibody (e.g., an anti-TIGIT antagonist antibody as disclosed herein, e.g., tiragolumab) is a dose (e.g., a fixed dose) of between about 30 mg to about 1200 mg (e.g., between about 30 mg to about 1100 mg, e.g., between about 60 mg to about 1000 mg, e.g., between about 100 mg to about 900 mg, e.g., between about 200 mg to about 800 mg, e.g., between about 300 mg to about 800 mg, e.g., between about 400 mg to about 800 mg, e.g., between about 400 mg to about 750 mg, e.g., between about 450 mg to about 750 mg, e.g., between about 500 mg to about 700 mg, e.g., between about 550 mg to about 650 mg, e.g., 600 mg±10 mg, e.g., 600±6 mg, e.g., 600±5 mg, e.g., 600±3 mg, e.g., 600±1 mg, e.g., 600±0.5 mg, e.g., 600 mg) every three weeks. In some instances, the effective amount of the anti-TIGIT antagonist antibody (e.g., an anti-TIGIT antagonist antibody as disclosed herein, e.g., tiragolumab) is a dose of between about 30 mg to about 600 mg (e.g., between about 50 mg to between 600 mg, e.g., between about 60 mg to about 600 mg, e.g., between about 100 mg to about 600 mg, e.g., between about 200 mg to about 600 mg, e.g., between about 200 mg to about 550 mg, e.g., between about 250 mg to about 500 mg, e.g., between about 300 mg to about 450 mg, e.g., between about 350 mg to about 400 mg, e.g., about 375 mg) every three weeks. In some instances, the effective amount of the anti-TIGIT antagonist antibody (e.g., an anti-TIGIT antagonist antibody as disclosed herein, e.g., tiragolumab) is a dose (e.g., a fixed dose) of between 30 mg to 1200 mg (e.g., between 30 mg to 1100 mg, e.g., between 60 mg to 1000 mg, e.g., between 100 mg to 900 mg, e.g., between 200 mg to 800 mg, e.g., between 300 mg to 800 mg, e.g., between 400 mg to 800 mg, e.g., between 400 mg to 750 mg, e.g., between 450 mg to 750 mg, e.g., between 500 mg to 700 mg, e.g., between 550 mg to 650 mg, e.g., 600 mg±10 mg, e.g., 600±6 mg, e.g., 600±5 mg, e.g., 600±3 mg, e.g., 600±1 mg, e.g., 600±0.5 mg, e.g., 600 mg) every three weeks. In some instances, the effective amount of the anti-TIGIT antagonist antibody (e.g., an anti-TIGIT antagonist antibody as disclosed herein, e.g., tiragolumab) is a dose of between 30 mg to 600 mg (e.g., between 50 mg to between 600 mg, e.g., between 60 mg to 600 mg, e.g., between 100 mg to 600 mg, e.g., between 200 mg to 600 mg, e.g., between 200 mg to 550 mg, e.g., between 250 mg to 500 mg, e.g., between 300 mg to 450 mg, e.g., between 350 mg to 400 mg, e.g., 375 mg) every three weeks. In some instances, the effective amount of the anti-TIGIT antagonist antibody (e.g., an anti-TIGIT antagonist antibody as disclosed herein, e.g., tiragolumab) is a dose of about 600 mg every three weeks. In some instances, effective amount of the anti-TIGIT antagonist antibody (e.g., an anti-TIGIT antagonist antibody as disclosed herein, e.g., tiragolumab) is a dose of 600 mg every three weeks. In some instances, the dose of the anti-TIGIT antagonist antibody (e.g., an anti-TIGIT antagonist antibody as disclosed herein, e.g., tiragolumab) is to be administered in a combination therapy (e.g., a combination treatment with a PD-1 axis binding antagonist (e.g., an anti-PD-L1 antagonist antibody (e.g., atezolizumab) or an anti-PD-1 antagonist antibody (e.g., MDX-1106 (nivolumab) or MK-3475 (pembrolizumab, previously known as lambrolizumab))) may be reduced as compared to a standard dose of the anti-TIGIT antagonist antibody is to be administered as a monotherapy.

In some instances, the effective amount of the PD-1 axis binding antagonist (e.g., anti-PD-L1 antagonist antibody (e.g., atezolizumab)) is a dose (e.g., a fixed dose) of between about 80 mg to about 2000 mg (e.g., between about 80 mg to about 1950 mg, e.g., between about 80 mg to about 1900 mg, e.g., between about 80 mg to about 1800 mg, e.g., between about 100 mg to about 1700 mg, e.g., between about 200 mg to about 1600 mg, e.g., between about 300 mg to about 1400 mg, e.g., between about 400 mg to about 1300 mg, e.g., between about 500 mg to about 1200 mg, e.g., between about 600 mg to about 1100 mg, e.g., between about 700 mg to about 1000 mg, e.g., between about 740 mg to about 940 mg, e.g., between about 790 mg to about 890 mg, e.g., between about 815 mg to about 865 mg, e.g., between about 830 mg to about 850 mg, e.g., 840 mg±5 mg, e.g., 840±2.5 mg, e.g., 840±1.0 mg, e.g., 840±0.5 mg, e.g., 840 mg) every two weeks. In some instances, the effective amount of the PD-1 axis binding antagonist (e.g., anti-PD-L1 antagonist antibody (e.g., atezolizumab)) is a dose of between about 80 mg to about 2000 mg (e.g., between about 100 mg to about 2000 mg, e.g., between about 200 mg to about 1900 mg, e.g., between about 300 mg to about 1700 mg, e.g., between about 400 mg to about 1600 mg, e.g., between about 500 mg to about 1600 mg, e.g., between about 600 mg to about 1600 mg, e.g., between about 700 mg to about 1600 mg, e.g., between about 800 mg to about 1600 mg, e.g., between about 900 mg to about 1500 mg, e.g., between about 1000 mg to about 1400 mg, e.g., between about 1050 mg to about 1350 mg, e.g., between about 1100 mg to about 1300 mg, e.g., between about 1150 mg to about 1250 mg, e.g., between about 1175 mg to about 1225 mg, e.g., between about 1190 mg to about 1210 mg, e.g., 1200 mg±5 mg, e.g., 1200±2.5 mg, e.g., 1200±1.0 mg, e.g., 1200±0.5 mg, e.g., 1200 mg) every three weeks. In some instances, the effective amount of the PD-1 axis binding antagonist (e.g., anti-PD-L1 antagonist antibody (e.g., atezolizumab)) is a dose of between about 80 mg to about 2000 mg (e.g., between about 100 mg to about 2000 mg, e.g., between about 200 mg to about 2000 mg, e.g., between about 300 mg to about 2000 mg, e.g., between about 400 mg to about 2000 mg, e.g., between about 500 mg to about 2000 mg, e.g., between about 600 mg to about 1900 mg, e.g., between about 700 mg to about 1800 mg, e.g., between about 800 mg to about 1800 mg, e.g., between about 900 mg to about 1800 mg, e.g., between about 1000 mg to about 1800 mg, e.g., between about 1100 mg to about 1800 mg, e.g., between about 1200 mg to about 1800 mg, e.g., between about 1300 mg to about 1800 mg, e.g., between about 1400 mg to about 1800 mg, e.g., between about 1500 mg to about 1800 mg, e.g., between about 1580 mg to about 1780 mg, e.g., between about 1630 mg to about 1730 mg, e.g., between about 1655 mg to about 1705 mg, e.g., between about 1670 mg to about 1690 mg, e.g., 1680 mg±5 mg, e.g., 1680±2.5 mg, e.g., 1680±1.0 mg, e.g., 1680±0.5 mg, e.g., 1680 mg) every four weeks. In some instances, the effective amount of the PD-1 axis binding antagonist (e.g., anti-PD-L1 antagonist antibody (e.g., atezolizumab)) is a dose (e.g., a fixed dose) of between 80 mg to 2000 mg (e.g., between 80 mg to 1950 mg, e.g., between 80 mg to 1900 mg, e.g., between 80 mg to 1800 mg, e.g., between 100 mg to 1700 mg, e.g., between 200 mg to 1600 mg, e.g., between 300 mg to 1400 mg, e.g., between 400 mg to 1300 mg, e.g., between 500 mg to 1200 mg, e.g., between 600 mg to 1100 mg, e.g., between 700 mg to 1000 mg, e.g., between 740 mg to 940 mg, e.g., between 790 mg to 890 mg, e.g., between 815 mg to 865 mg, e.g., between 830 mg to 850 mg, e.g., 840 mg±5 mg, e.g., 840±2.5 mg, e.g., 840±1.0 mg, e.g., 840±0.5 mg, e.g., 840 mg) every two weeks. In some instances, the effective amount of the PD-1 axis binding antagonist (e.g., anti-PD-L1 antagonist antibody (e.g., atezolizumab)) is a dose of between 80 mg to 2000 mg (e.g., between 100 mg to 2000 mg, e.g., between 200 mg to 1900 mg, e.g., between 300 mg to 1700 mg, e.g., between 400 mg to 1600 mg, e.g., between 500 mg to 1600 mg, e.g., between 600 mg to 1600 mg, e.g., between 700 mg to 1600 mg, e.g., between 800 mg to 1600 mg, e.g., between 900 mg to 1500 mg, e.g., between 1000 mg to 1400 mg, e.g., between 1050 mg to 1350 mg, e.g., between 1100 mg to 1300 mg, e.g., between 1150 mg to 1250 mg, e.g., between 1175 mg to 1225 mg, e.g., between 1190 mg to 1210 mg, e.g., 1200 mg ±5 mg, e.g., 1200±2.5 mg, e.g., 1200±1.0 mg, e.g., 1200±0.5 mg, e.g., 1200 mg) every three weeks. In some instances, the effective amount of the PD-1 axis binding antagonist (e.g., anti-PD-L1 antagonist antibody (e.g., atezolizumab)) is a dose of between 80 mg to 2000 mg (e.g., between 100 mg to 2000 mg, e.g., between 200 mg to 2000 mg, e.g., between 300 mg to 2000 mg, e.g., between 400 mg to 2000 mg, e.g., between 500 mg to 2000 mg, e.g., between 600 mg to 1900 mg, e.g., between 700 mg to 1800 mg, e.g., between 800 mg to 1800 mg, e.g., between 900 mg to 1800 mg, e.g., between 1000 mg to 1800 mg, e.g., between 1100 mg to 1800 mg, e.g., between 1200 mg to 1800 mg, e.g., between 1300 mg to 1800 mg, e.g., between 1400 mg to 1800 mg, e.g., between 1500 mg to 1800 mg, e.g., between 1580 mg to 1780 mg, e.g., between 1630 mg to 1730 mg, e.g., between 1655 mg to 1705 mg, e.g., between 1670 mg to 1690 mg, e.g., 1680 mg±5 mg, e.g., 1680±2.5 mg, e.g., 1680±1.0 mg, e.g., 1680±0.5 mg, e.g., 1680 mg) every four weeks. In some instances, the effective amount of the PD-1 axis binding antagonist (e.g., anti-PD-L1 antagonist antibody (e.g., atezolizumab)) is a dose of about 840 mg every two weeks. In some instances, the effective amount of the PD-1 axis binding antagonist (e.g., anti-PD-L1 antagonist antibody (e.g., atezolizumab)) is a dose of 840 mg every two weeks. In some instances, the effective amount of the PD-1 axis binding antagonist (e.g., anti-PD-L1 antagonist antibody (e.g., atezolizumab)) is a dose of about 1200 mg every three weeks. In some instances, the effective amount of the PD-1 axis binding antagonist (e.g., anti-PD-L1 antagonist antibody (e.g., atezolizumab)) is a dose of 1200 mg every three weeks. In some instances, the effective amount of the PD-1 axis binding antagonist (e.g., anti-PD-L1 antagonist antibody (e.g., atezolizumab)) is a dose of about 1680 mg every four weeks. In some instances, the effective amount of the PD-1 axis binding antagonist (e.g., anti-PD-L1 antagonist antibody (e.g., atezolizumab)) is a dose of 1680 mg every four weeks. In some instances, the dose of the PD-1 axis binding antagonist (e.g., anti-PD-L1 antagonist antibody (e.g., atezolizumab)) to be administered in a combination therapy (e.g., a combination treatment with an anti-TIGIT antagonist antibody, such as an anti-TIGIT antagonist antibody disclosed herein, e.g., tiragolumab) may be reduced as compared to a standard dose of the anti-PD-L1 antagonist antibody to be administered as a monotherapy.

In some instances, the effective amount of the PD-1 axis binding antagonist (e.g., anti-PD-1 antagonist antibody (e.g., pembrolizumab)) is a dose (e.g., a fixed dose) of between about 20 mg to about 1000 mg (e.g., between about 40 mg to about 900 mg, e.g., between about 60 mg to about 800 mg, e.g., between about 80 mg to about 700 mg, e.g., between about 80 mg to about 600 mg, e.g., between about 100 mg to about 500 mg, e.g., between about 120 mg to about 400 mg, e.g., between about 140 mg to about 300 mg, e.g., between about 160 mg to about 350 mg, e.g., between about 180 mg to about 300 mg, e.g., between about 180 mg to about 250 mg, e.g., between about 180 mg to about 220 mg, e.g., between about 190 mg to about 210 mg, e.g., 200 mg±5 mg, e.g., 200±2.5 mg, e.g., 200±1.0 mg, e.g., 200±0.5 mg, e.g., 200 mg) every three weeks. In some instances, the effective amount of the PD-1 axis binding antagonist (e.g., anti-PD-1 antagonist antibody (e.g., pembrolizumab)) is a dose (e.g., a fixed dose) of between 20 mg to 1000 mg (e.g., between 40 mg to 900 mg, e.g., between 60 mg to 800 mg, e.g., between 80 mg to 700 mg, e.g., between 80 mg to 600 mg, e.g., between 100 mg to 500 mg, e.g., between 120 mg to 400 mg, e.g., between 140 mg to 300 mg, e.g., between 160 mg to 350 mg, e.g., between 180 mg to 300 mg, e.g., between 180 mg to 250 mg, e.g., between 180 mg to 220 mg, e.g., between 190 mg to 210 mg, e.g., 200 mg±5 mg, e.g., 200±2.5 mg, e.g., 200±1.0 mg, e.g., 200±0.5 mg, e.g., 200 mg) every three weeks. In some instances, the effective amount of the PD-1 axis binding antagonist (e.g., anti-PD-1 antagonist antibody (e.g., pembrolizumab)) is a dose of about 200 mg every three weeks. In some instances, the effective amount of the PD-1 axis binding antagonist (e.g., anti-PD-1 antagonist antibody (e.g., pembrolizumab)) is a dose of 200 mg every three weeks. In some instances, the dose of the PD-1 axis binding antagonist (e.g., anti-PD-1 antagonist antibody (e.g., pembrolizumab)) administered in a combination therapy (e.g., a combination treatment with an anti-TIGIT antagonist antibody, such as an anti-TIGIT antagonist antibody disclosed herein, e.g., tiragolumab) may be reduced as compared to a standard dose of the anti-PD-L1 antagonist antibody administered as a monotherapy.

In some instances, the effective amount of the PD-1 axis binding antagonist (e.g., anti-PD-1 antagonist antibody (e.g., nivolumab)) is a dose (e.g., a fixed dose) of between about 20 mg to about 1000 mg (e.g., between about 40 mg to about 900 mg, e.g., between about 60 mg to about 800 mg, e.g., between about 80 mg to about 700 mg, e.g., between about 80 mg to about 600 mg, e.g., between about 100 mg to about 500 mg, e.g., between about 120 mg to about 400 mg, e.g., between about 140 mg to about 300 mg, e.g., between about 160 mg to about 350 mg, e.g., between about 180 mg to about 300 mg, e.g., between about 200 mg to about 280 mg, e.g., between about 220 mg to about 260 mg, e.g., between about 230 mg to about 250 mg, e.g., 240 mg±5 mg, e.g., 240±2.5 mg, e.g., 240±1.0 mg, e.g., 240±0.5 mg, e.g., 240 mg) every two weeks. In some instances, the effective amount of the PD-1 axis binding antagonist (e.g., anti-PD-1 antagonist antibody (e.g., nivolumab)) is a dose of about 240 mg every two weeks. In some instances, the effective amount of the PD-1 axis binding antagonist (e.g., anti-PD-1 antagonist antibody (e.g., nivolumab)) is a dose of 240 mg every two weeks. In some instances, the effective amount of the PD-1 axis binding antagonist (e.g., anti-PD-1 antagonist antibody (e.g., nivolumab)) is a dose of between about 100 mg to about 1000 mg (e.g., between about 200 mg to about 900 mg, e.g., between about 300 mg to about 800 mg, e.g., between about 400 mg to about 700 mg, e.g., between about 400 mg to about 600 mg, e.g., between about 400 mg to about 550 mg, e.g., between about 420 mg to about 540 mg, e.g., between about 440 mg to about 520 mg, e.g., between about 460 mg to about 500 mg, e.g., between about 470 mg to about 490 mg, e.g., 480 mg±5 mg, e.g., 480±2.5 mg, e.g., 480±1.0 mg, e.g., 480±0.5 mg, e.g., 480 mg) every four weeks. In some instances, the effective amount of the PD-1 axis binding antagonist (e.g., anti-PD-1 antagonist antibody (e.g., nivolumab)) is a dose (e.g., a fixed dose) of between 20 mg to 1000 mg (e.g., between 40 mg to 900 mg, e.g., between 60 mg to 800 mg, e.g., between 80 mg to 700 mg, e.g., between 80 mg to 600 mg, e.g., between 100 mg to 500 mg, e.g., between 120 mg to 400 mg, e.g., between 140 mg to 300 mg, e.g., between 160 mg to 350 mg, e.g., between 180 mg to 300 mg, e.g., between 200 mg to 280 mg, e.g., between 220 mg to 260 mg, e.g., between 230 mg to 250 mg, e.g., 240 mg±5 mg, e.g., 240±2.5 mg, e.g., 240±1.0 mg, e.g., 240±0.5 mg, e.g., 240 mg) every two weeks. In some instances, the effective amount of the PD-1 axis binding antagonist (e.g., anti-PD-1 antagonist antibody (e.g., nivolumab)) is a dose of 240 mg every two weeks. In some instances, the effective amount of the PD-1 axis binding antagonist (e.g., anti-PD-1 antagonist antibody (e.g., nivolumab)) is a dose of 240 mg every two weeks. In some instances, the effective amount of the PD-1 axis binding antagonist (e.g., anti-PD-1 antagonist antibody (e.g., nivolumab)) is a dose of between 100 mg to 1000 mg (e.g., between 200 mg to 900 mg, e.g., between 300 mg to 800 mg, e.g., between 400 mg to 700 mg, e.g., between 400 mg to 600 mg, e.g., between 400 mg to 550 mg, e.g., between 420 mg to 540 mg, e.g., between 440 mg to 520 mg, e.g., between 460 mg to 500 mg, e.g., between 470 mg to 490 mg, e.g., 480 mg±5 mg, e.g., 480±2.5 mg, e.g., 480±1.0 mg, e.g., 480±0.5 mg, e.g., 480 mg) every four weeks. In some instances, the effective amount of the PD-1 axis binding antagonist (e.g., anti-PD-1 antagonist antibody (e.g., nivolumab)) is a dose of about 480 mg every four weeks. In some instances, the effective amount of the PD-1 axis binding antagonist (e.g., anti-PD-1 antagonist antibody (e.g., nivolumab)) is a dose of 480 mg every four weeks. In some instances, the dose of the PD-1 axis binding antagonist (e.g., anti-PD-1 antagonist antibody (e.g., nivolumab)) administered in a combination therapy (e.g., a combination treatment with an anti-TIGIT antagonist antibody, such as an anti-TIGIT antagonist antibody disclosed herein, e.g., tiragolumab) may be reduced as compared to a standard dose of the anti-PD-L1 antagonist antibody administered as a monotherapy.

In some instances, the effective amount of the PD-1 axis binding antagonist (e.g., anti-PD-L1 antagonist antibody (e.g., atezolizumab)) is a dose of between about 0.01 mg/kg to about 50 mg/kg of the subject's body weight (e.g., between about 0.01 mg/kg to about 45 mg/kg, e.g., between about 0.1 mg/kg to about 40 mg/kg, e.g., between about 1 mg/kg to about 35 mg/kg, e.g., between about 2.5 mg/kg to about 30 mg/kg, e.g., between about 5 mg/kg to about 25 mg/kg, e.g., between about 10 mg/kg to about 20 mg/kg, e.g., between about 12.5 mg/kg to about 15 mg/kg, e.g., about 15±2 mg/kg, about 15±1 mg/kg, about 15±0.5 mg/kg, about 15±0.2 mg/kg, or about 15±0.1 mg/kg, e.g., about 15 mg/kg) every three weeks. In some instances, the effective amount of the PD-1 axis binding antagonist (e.g., anti-PD-L1 antagonist antibody (e.g., atezolizumab)) is a dose of between about 0.01 mg/kg to about 15 mg/kg of the subject's body weight (e.g., between about 0.1 mg/kg to about 15 mg/kg, e.g., between about 0.5 mg/kg to about 15 mg/kg, e.g., between about 1 mg/kg to about 15 mg/kg, e.g., between about 2.5 mg/kg to about 15 mg/kg, e.g., between about 5 mg/kg to about 15 mg/kg, e.g., between about 7.5 mg/kg to about 15 mg/kg, e.g., between about 10 mg/kg to about 15 mg/kg, e.g., between about 12.5 mg/kg to about 15 mg/kg, e.g., between about 14 mg/kg to about 15 mg/kg, e.g., about 15±1 mg/kg, e.g., about 15±0.5 mg/kg, e.g., about 15±0.2 mg/kg, e.g., about 15±0.1 mg/kg, e.g., about 15 mg/kg) every three weeks. In some instances, effective amount of anti-PD-L1 antagonist antibody (e.g., atezolizumab) is a dose of about 15 mg/kg to be administered every three weeks. In some instances, the effective amount of the PD-1 axis binding antagonist (e.g., anti-PD-L1 antagonist antibody (e.g., atezolizumab)) is a dose of between 0.01 mg/kg to 50 mg/kg of the subject's body weight (e.g., between 0.01 mg/kg to 45 mg/kg, e.g., between 0.1 mg/kg to 40 mg/kg, e.g., between 1 mg/kg to 35 mg/kg, e.g., between 2.5 mg/kg to 30 mg/kg, e.g., between 5 mg/kg to 25 mg/kg, e.g., between 10 mg/kg to 20 mg/kg, e.g., between 12.5 mg/kg to 15 mg/kg, e.g., 15±2 mg/kg, 15±1 mg/kg, 15±0.5 mg/kg, 15±0.2 mg/kg, or 15±0.1 mg/kg, e.g., 15 mg/kg) every three weeks. In some instances, the effective amount of the PD-1 axis binding antagonist (e.g., anti-PD-L1 antagonist antibody (e.g., atezolizumab)) is a dose of between 0.01 mg/kg to 15 mg/kg of the subject's body weight (e.g., between 0.1 mg/kg to 15 mg/kg, e.g., between 0.5 mg/kg to 15 mg/kg, e.g., between 1 mg/kg to 15 mg/kg, e.g., between 2.5 mg/kg to 15 mg/kg, e.g., between 5 mg/kg to 15 mg/kg, e.g., between 7.5 mg/kg to 15 mg/kg, e.g., between 10 mg/kg to 15 mg/kg, e.g., between 12.5 mg/kg to 15 mg/kg, e.g., between 14 mg/kg to 15 mg/kg, e.g., 15±1 mg/kg, e.g., 15±0.5 mg/kg, e.g., 15±0.2 mg/kg, e.g., 15±0.1 mg/kg, e.g., 15 mg/kg) every three weeks. In some instances, effective amount of anti-PD-L1 antagonist antibody (e.g., atezolizumab) is a dose of 15 mg/kg to be administered every three weeks. In some instances, the dose of the PD-1 axis binding antagonist (e.g., an anti-PD-L1 antagonist antibody (e.g., atezolizumab) or an anti-PD-1 antagonist antibody (e.g., MDX-1106 (nivolumab) or MK-3475 (pembrolizumab, previously known as lambrolizumab))) is administered in a combination therapy (e.g., a combination treatment with an anti-TIGIT antagonist antibody, such as an anti-TIGIT antagonist antibody disclosed herein, e.g., tiragolumab) may be reduced as compared to a standard dose of the PD-1 axis binding antagonist (e.g., anti-PD-L1 antagonist antibody (e.g., atezolizumab) or anti-PD-1 antagonist antibody (e.g., MDX-1106 (nivolumab) or MK-3475 (pembrolizumab, previously known as lambrolizumab))) administered as a monotherapy.

The anti-TIGIT antagonist antibody (e.g., an anti-TIGIT antagonist antibody as disclosed herein, e.g., tiragolumab) and the PD-1 axis binding antagonist (e.g., an anti-PD-L1 antagonist antibody (e.g., atezolizumab) or an anti-PD-1 antagonist antibody (e.g., MDX-1106 (nivolumab) or MK-3475 (pembrolizumab, previously known as lambrolizumab))) may be administered in one or more dosing cycles (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 or more dosing cycles). In some instances, the dosing cycles of the anti-TIGIT antagonist antibody (e.g., an anti-TIGIT antagonist antibody as disclosed herein, e.g., tiragolumab) and the PD-1 axis binding antagonist (e.g., an anti-PD-L1 antagonist antibody (e.g., atezolizumab) or an anti-PD-1 antagonist antibody (e.g., MDX-1106 (nivolumab) or MK-3475 (pembrolizumab, previously known as lambrolizumab))) continue until there is a loss of clinical benefit (e.g., confirmed disease progression, drug resistance, death, or unacceptable toxicity). In some instances, the length of each dosing cycle is about 14 to 28 days (e.g., 14 days, 15 days, 16 days, 17 days, 18 days, 19 days, 20 days, 21 days, 22 days, 23 days, 24 days, 25 days, 26 days, 27 days, or 28 days). In some instances, the length of each dosing cycle is about 21 days. In some instances, the length of each dosing cycle is about 14 days. In some instances, the length of each dosing cycle is about 28 days. In some instances, the anti-TIGIT antagonist antibody (e.g., an anti-TIGIT antagonist antibody as disclosed herein, e.g., tiragolumab) is to be administered on about Day 1 (e.g., Day 1±3 days) of each dosing cycle. For example, the anti-TIGIT antagonist antibody (e.g., an anti-TIGIT antagonist antibody as disclosed herein, e.g., tiragolumab) is to be administered intravenously at a dose (e.g., a fixed dose) of about 600 mg on Day 1 of each 21-day cycle (i.e., at a dose of about 600 mg every three weeks). Similarly, in some instances, the PD-1 axis binding antagonist (e.g., an anti-PD-L1 antagonist antibody (e.g., atezolizumab) or an anti-PD-1 antagonist antibody (e.g., MDX-1106 (nivolumab) or MK-3475 (pembrolizumab, previously known as lambrolizumab))) is to be administered on about Day 1 (e.g., Day 1±3 days) of each dosing cycle. For example, the PD-1 axis binding antagonist (e.g., anti-PD-L1 antagonist antibody (e.g., atezolizumab)) is to be administered intravenously at a dose (e.g., a fixed dose) of about 1200 mg on Day 1 of each 21-day cycle (i.e., at a dose of about 1200 mg every three weeks). For example, the PD-1 axis binding antagonist (e.g., anti-PD-1 antagonist antibody (e.g., pembrolizumab)) is to be administered intravenously at a dose of about 200 mg on Day 1 of each 21-day cycle (i.e., at a dose of about 200 mg every three weeks). For example, the PD-1 axis binding antagonist (e.g., anti-PD-1 antagonist antibody (e.g., nivolumab)) is to be administered intravenously at a dose of about 240 mg on Day 1 of each 14-day cycle (i.e., at a dose of about 240 mg every two weeks). For example, the PD-1 axis binding antagonist (e.g., anti-PD-1 antagonist antibody (e.g., nivolumab)) is to be administered intravenously at a dose of about 480 mg on Day 1 of each 28-day cycle (i.e., at a dose of about 480 mg every four weeks). In some instances, both the anti-TIGIT antagonist antibody (e.g., an anti-TIGIT antagonist antibody as disclosed herein, e.g., tiragolumab) and the PD-1 axis binding antagonist (e.g., an anti-PD-L1 antagonist antibody (e.g., atezolizumab) or an anti-PD-1 antagonist antibody (e.g., MDX-1106 (nivolumab) or MK-3475 (pembrolizumab, previously known as lambrolizumab))) are to be administered on about Day 1 (e.g., Day 1±3 days) of each dosing cycle. In some instances, both the anti-TIGIT antagonist antibody (e.g., an anti-TIGIT antagonist antibody as disclosed herein, e.g., tiragolumab) and the PD-1 axis binding antagonist (e.g., an anti-PD-L1 antagonist antibody (e.g., atezolizumab) or an anti-PD-1 antagonist antibody (e.g., MDX-1106 (nivolumab) or MK-3475 (pembrolizumab, previously known as lambrolizumab))) are to be administered on about Day 1 (e.g., Day 1±3 days) of the first dosing cycle. For example, the anti-TIGIT antagonist antibody (e.g., an anti-TIGIT antagonist antibody as disclosed herein, e.g., tiragolumab) is to be administered intravenously at a dose of about 600 mg on Day 1 of each 21-day cycle (i.e., at a dose of about 600 mg every three weeks), and the PD-1 axis binding antagonist (e.g., anti-PD-L1 antagonist antibody (e.g., nivolumab)) is administered intravenously at a dose of about 240 mg on Day 1 of each 14-day cycle (i.e., at a dose of about 240 mg every two weeks). For example, the anti-TIGIT antagonist antibody (e.g., an anti-TIGIT antagonist antibody as disclosed herein, e.g., tiragolumab) is to be administered intravenously at a dose of about 600 mg on Day 1 of each 21-day cycle (i.e., at a dose of about 600 mg every three weeks), and the PD-1 axis binding antagonist (e.g., anti-PD-L1 antagonist antibody (e.g., nivolumab)) is administered intravenously at a dose of about 480 mg on Day 1 of each 28-day cycle (i.e., at a dose of about 480 mg every four weeks). In some examples, the PD-1 axis binding antagonist (e.g., anti-PD-L1 antagonist antibody (e.g., atezolizumab)) is to be administered intravenously at a dose (e.g., a fixed dose) of 1200 mg on Day 1 of each 21-day cycle (i.e., at a dose of 1200 mg every three weeks). For example, the PD-1 axis binding antagonist (e.g., anti-PD-1 antagonist antibody (e.g., pembrolizumab)) is to be administered intravenously at a dose of 200 mg on Day 1 of each 21-day cycle (i.e., at a dose of 200 mg every three weeks). For example, the PD-1 axis binding antagonist (e.g., anti-PD-1 antagonist antibody (e.g., nivolumab)) is to be administered intravenously at a dose of 240 mg on Day 1 of each 14-day cycle (i.e., at a dose of 240 mg every two weeks). For example, the PD-1 axis binding antagonist (e.g., anti-PD-1 antagonist antibody (e.g., nivolumab)) is to be administered intravenously at a dose of 480 mg on Day 1 of each 28-day cycle (i.e., at a dose of 480 mg every four weeks). In some instances, both the anti-TIGIT antagonist antibody (e.g., an anti-TIGIT antagonist antibody as disclosed herein, e.g., tiragolumab) and the PD-1 axis binding antagonist (e.g., an anti-PD-L1 antagonist antibody (e.g., atezolizumab) or an anti-PD-1 antagonist antibody (e.g., MDX-1106 (nivolumab) or MK-3475 (pembrolizumab, previously known as lambrolizumab))) are to be administered on Day 1 (e.g., Day 1±3 days) of each dosing cycle. In some instances, both the anti-TIGIT antagonist antibody (e.g., an anti-TIGIT antagonist antibody as disclosed herein, e.g., tiragolumab) and the PD-1 axis binding antagonist (e.g., an anti-PD-L1 antagonist antibody (e.g., atezolizumab) or an anti-PD-1 antagonist antibody (e.g., MDX-1106 (nivolumab) or MK-3475 (pembrolizumab, previously known as lambrolizumab))) are to be administered on Day 1 (e.g., Day 1 3 days) of the first dosing cycle. For example, the anti-TIGIT antagonist antibody (e.g., an anti-TIGIT antagonist antibody as disclosed herein, e.g., tiragolumab) is to be administered intravenously at a dose of 600 mg on Day 1 of each 21-day cycle (i.e., at a dose of 600 mg every three weeks), and the PD-1 axis binding antagonist (e.g., anti-PD-L1 antagonist antibody (e.g., nivolumab)) is administered intravenously at a dose of 240 mg on Day 1 of each 14-day cycle (i.e., at a dose of 240 mg every two weeks). For example, the anti-TIGIT antagonist antibody (e.g., an anti-TIGIT antagonist antibody as disclosed herein, e.g., tiragolumab) is to be administered intravenously at a dose of 600 mg on Day 1 of each 21-day cycle (i.e., at a dose of 600 mg every three weeks), and the PD-1 axis binding antagonist (e.g., anti-PD-L1 antagonist antibody (e.g., nivolumab)) is administered intravenously at a dose of 480 mg on Day 1 of each 28-day cycle (i.e., at a dose of 480 mg every four weeks).

In some instances, the anti-TIGIT antagonist antibody (e.g., an anti-TIGIT antagonist antibody as disclosed herein, e.g., tiragolumab) is to be administered to the subject or population of subjects by intravenous infusion over about 60±15 minutes (e.g., about 50 minutes, about 51 minutes, about 52 minutes, about 53 minutes, about 54 minutes, about 55 minutes, about 56 minutes, about 57 minutes, about 58 minutes, about 59 minutes, about 60 minutes, about 61 minutes, about 62 minutes, about 63 minutes, about 64 minutes, about 65 minutes, about 66 minutes, about 67 minutes, about 68 minutes, about 69 minutes, or about 70 minutes). In some instances, the PD-1 axis binding antagonist (e.g., an anti-PD-L1 antagonist antibody (e.g., atezolizumab) or an anti-PD-1 antagonist antibody (e.g., MDX-1106 (nivolumab) or MK-3475 (pembrolizumab, previously known as lambrolizumab))) is to be administered to the subject or population of subjects by intravenous infusion over about 60±15 minutes (e.g., about 45 minutes, about 46 minutes, about 47 minutes, about 48 minutes, about 49 minutes, about 50 minutes, about 51 minutes, about 52 minutes, about 53 minutes, about 54 minutes, about 55 minutes, about 56 minutes, about 57 minutes, about 58 minutes, about 59 minutes, about 60 minutes, about 61 minutes, about 62 minutes, about 63 minutes, about 64 minutes, about 65 minutes, about 66 minutes, about 67 minutes, about 68 minutes, about 69 minutes, about 70 minutes, about 71 minutes, about 72 minutes, about 73 minutes, about 74 minutes, or about 75 minutes).

In some instances, the anti-TIGIT antagonist antibody (e.g., an anti-TIGIT antagonist antibody as disclosed herein, e.g., tiragolumab) is to be administered to the subject or population of subjects by intravenous infusion over about 30±10 minutes (e.g., about 20 minutes, about 21 minutes, about 22 minutes, about 23 minutes, about 24 minutes, about 25 minutes, about 26 minutes, about 27 minutes, about 28 minutes, about 29 minutes, about 30 minutes, about 31 minutes, about 32 minutes, about 33 minutes, about 34 minutes, about 35 minutes, about 36 minutes, about 37 minutes, about 38 minutes, about 39 minutes, or about 40 minutes). In some instances, the PD-1 axis binding antagonist (e.g., an anti-PD-L1 antagonist antibody (e.g., atezolizumab) or an anti-PD-1 antagonist antibody (e.g., MDX-1106 (nivolumab) or MK-3475 (pembrolizumab, previously known as lambrolizumab))) is to be administered to the subject or population of subjects by intravenous infusion over about 30±10 minutes (e.g., about 20 minutes, about 21 minutes, about 22 minutes, about 23 minutes, about 24 minutes, about 25 minutes, about 26 minutes, about 27 minutes, about 28 minutes, about 29 minutes, about 30 minutes, about 31 minutes, about 32 minutes, about 33 minutes, about 34 minutes, about 35 minutes, about 36 minutes, about 37 minutes, about 38 minutes, about 39 minutes, or about 40 minutes).

In some instances, the anti-TIGIT antagonist antibody (e.g., an anti-TIGIT antagonist antibody as disclosed herein, e.g., tiragolumab) is to be administered to the subject or population of subjects before the PD-1 axis binding antagonist (e.g., an anti-PD-L1 antagonist antibody (e.g., atezolizumab) or an anti-PD-1 antagonist antibody (e.g., MDX-1106 (nivolumab) or MK-3475 (pembrolizumab, previously known as lambrolizumab))). In some instances, for example, following administration of the anti-TIGIT antagonist antibody and before administration of the PD-1 axis binding antagonist (e.g., anti-PD-L1 antagonist antibody (e.g., atezolizumab) or anti-PD-1 antagonist antibody (e.g., MDX-1106 (nivolumab) or MK-3475 (pembrolizumab, previously known as lambrolizumab))), the method includes an intervening first observation period. In some instances, the method further includes a second observation period following administration of the PD-1 axis binding antagonist (e.g., anti-PD-L1 antagonist antibody (e.g., atezolizumab) or anti-PD-1 antagonist antibody (e.g., MDX-1106 (nivolumab) or MK-3475 (pembrolizumab, previously known as lambrolizumab))). In some instances, the method includes both a first observation period following administration of the anti-TIGIT antagonist antibody and second observation period following administration of the PD-1 axis binding antagonist (e.g., anti-PD-L1 antagonist antibody (e.g., atezolizumab) or anti-PD-1 antagonist antibody (e.g., MDX-1106 (nivolumab) or MK-3475 (pembrolizumab, previously known as lambrolizumab))). In some instances, the first and second observation periods are each between about 30 minutes to about 60 minutes in length. In instances in which the first and second observation periods are each about 60 minutes in length, the method may include recording the subject or population of subjects' vital signs (e.g., pulse rate, respiratory rate, blood pressure, and temperature) at about 30±10 minutes after administration of the anti-TIGIT antagonist antibody and PD-1 axis binding antagonist (e.g., anti-PD-L1 antagonist antibody (e.g., atezolizumab) or anti-PD-1 antagonist antibody (e.g., MDX-1106 (nivolumab) or MK-3475 (pembrolizumab, previously known as lambrolizumab))) during the first and second observation periods, respectively. In instances in which the first and second observation periods are each about 30 minutes in length, the method may include recording the subject or population of subjects' vital signs (e.g., pulse rate, respiratory rate, blood pressure, and temperature) at about 15±10 minutes after administration of the anti-TIGIT antagonist antibody and PD-1 axis binding antagonist (e.g., anti-PD-L1 antagonist antibody (e.g., atezolizumab) or anti-PD-1 antagonist antibody (e.g., MDX-1106 (nivolumab) or MK-3475 (pembrolizumab, previously known as lambrolizumab))) during the first and second observation periods, respectively.

In other instances, the PD-1 axis binding antagonist (e.g., an anti-PD-L1 antagonist antibody (e.g., atezolizumab) or an anti-PD-1 antagonist antibody (e.g., MDX-1106 (nivolumab) or MK-3475 (pembrolizumab, previously known as lambrolizumab))) is to be administered to the subject or population of subjects before the anti-TIGIT antagonist antibody (e.g., an anti-TIGIT antagonist antibody as disclosed herein, e.g., tiragolumab). In some instances, for example, following administration of the PD-1 axis binding antagonist (e.g., anti-PD-L1 antagonist antibody (e.g., atezolizumab) or anti-PD-1 antagonist antibody (e.g., MDX-1106 (nivolumab) or MK-3475 (pembrolizumab, previously known as lambrolizumab))) and before administration of the anti-TIGIT antagonist antibody, the method includes an intervening first observation period. In some instances, the method includes a second observation period following administration of the anti-TIGIT antagonist antibody. In some instances, the method includes both a first observation period following administration of the PD-1 axis binding antagonist (e.g., anti-PD-L1 antagonist antibody (e.g., atezolizumab) or anti-PD-1 antagonist antibody (e.g., MDX-1106 (nivolumab) or MK-3475 (pembrolizumab, previously known as lambrolizumab))) and second observation period following administration of the anti-TIGIT antagonist antibody. In some instances, the first and second observation periods are each between about 30 minutes to about 60 minutes in length. In instances in which the first and second observation periods are each about 60 minutes in length, the method may include recording the subject or population of subjects' vital signs (e.g., pulse rate, respiratory rate, blood pressure, and temperature) at about 30±10 minutes after administration of the PD-1 axis binding antagonist (e.g., anti-PD-L1 antagonist antibody (e.g., atezolizumab) or anti-PD-1 antagonist antibody (e.g., MDX-1106 (nivolumab) or MK-3475 (pembrolizumab, previously known as lambrolizumab))) and anti-TIGIT antagonist antibody during the first and second observation periods, respectively. In instances in which the first and second observation periods are each about 30 minutes in length, the method may include recording the subject or population of subjects' vital signs (e.g., pulse rate, respiratory rate, blood pressure, and temperature) at about 15±10 minutes after administration of the PD-1 axis binding antagonist (e.g., anti-PD-L1 antagonist antibody (e.g., atezolizumab) or anti-PD-1 antagonist antibody (e.g., MDX-1106 (nivolumab) or MK-3475 (pembrolizumab, previously known as lambrolizumab))) and anti-TIGIT antagonist antibody during the first and second observation periods, respectively.

In other instances, the anti-TIGIT antagonist antibody (e.g., an anti-TIGIT antagonist antibody as disclosed herein, e.g., tiragolumab) and the PD-1 axis binding antagonist (e.g., anti-PD-L1 antagonist antibody (e.g., atezolizumab) or anti-PD-1 antagonist antibody (e.g., MDX-1106 (nivolumab) or MK-3475 (pembrolizumab, previously known as lambrolizumab))) is to be administered to the subject or population of subjects simultaneously. In some instances, for example, following administration of the anti-TIGIT antagonist antibody and the PD-1 axis binding antagonist (e.g., anti-PD-L1 antagonist antibody (e.g., atezolizumab) or anti-PD-1 antagonist antibody (e.g., MDX-1106 (nivolumab) or MK-3475 (pembrolizumab, previously known as lambrolizumab))) the method includes an observation period. In some instances, the observation period is between about 30 minutes to about 60 minutes in length. In instances in which the observation period is about 60 minutes in length, the method may include recording the subject's vital signs (e.g., pulse rate, respiratory rate, blood pressure, and temperature) at about 30±10 minutes after administration of the PD-1 axis binding antagonist (e.g., anti-PD-L1 antagonist antibody (e.g., atezolizumab) or anti-PD-1 antagonist antibody (e.g., MDX-1106 (nivolumab) or MK-3475 (pembrolizumab, previously known as lambrolizumab))) and anti-TIGIT antagonist antibody during the observation period. In instances in which the observation period is about 30 minutes in length, the method may include recording the subject's vital signs (e.g., pulse rate, respiratory rate, blood pressure, and temperature) at about 15±10 minutes after administration of the PD-1 axis binding antagonist (e.g., anti-PD-L1 antagonist antibody (e.g., atezolizumab) or anti-PD-1 antagonist antibody (e.g., MDX-1106 (nivolumab) or MK-3475 (pembrolizumab, previously known as lambrolizumab))) and anti-TIGIT antagonist antibody during the observation period.

In another aspect, the invention provides an anti-TIGIT antagonist antibody (e.g., an anti-TIGIT antagonist antibody disclosed herein, e.g., tiragolumab) and PD-1 axis binding antagonist (e.g., anti-PD-L1 antagonist antibody (e.g., atezolizumab)) for use in a method of treating a subject or population of subjects having a cancer with a detectable expression level of PD-L1 (e.g., cervical cancer, e.g., Stage IVB, metastatic, recurrent, or persistent cervical cancer, e.g., a metastatic and/or recurrent PD-L1-positive cervical carcinoma), wherein the method comprises administering to the subject or population of subjects one or more dosing cycles of an anti-TIGIT antagonist antibody at a dose (e.g., a fixed dose) of 600 mg every three weeks and atezolizumab at a dose (e.g., a fixed dose) of 1200 mg every three weeks, wherein the anti-TIGIT antagonist antibody comprises: a VH domain comprising the amino acid sequence of SEQ ID NO: 17 or 18; and a VL domain comprising the amino acid sequence of SEQ ID NO: 19, as described in further detail below. In some instances, the anti-TIGIT antagonist antibody has a VH domain having the amino acid sequence of SEQ ID NO: 17 and a VL domain having the amino acid sequence of SEQ ID NO: 19. In some instances, the anti-TIGIT antagonist antibody has a VH domain having the amino acid sequence of SEQ ID NO: 18 and a VL domain having the amino acid sequence of SEQ ID NO: 19.

In another aspect, the invention provides an anti-TIGIT antagonist antibody (e.g., an anti-TIGIT antagonist antibody disclosed herein, e.g., tiragolumab) and PD-1 axis binding antagonist (e.g., anti-PD-1 antagonist antibody (e.g., pembrolizumab)) for use in a method of treating a subject or population of subjects having a cancer with a detectable expression level of PD-L1 (e.g., cervical cancer, e.g., Stage IVB, metastatic, recurrent, or persistent cervical cancer, e.g., a metastatic and/or recurrent PD-L1-positive cervical carcinoma), wherein the method comprises administering to the subject or population of subjects one or more dosing cycles of an anti-TIGIT antagonist antibody at a dose (e.g., a fixed dose) of 600 mg every three weeks and pembrolizumab at a dose (e.g., a fixed dose) of 200 mg every three weeks, wherein the anti-TIGIT antagonist antibody comprises: a VH domain comprising the amino acid sequence of SEQ ID NO: 17 or 18; and a VL domain comprising the amino acid sequence of SEQ ID NO: 19, as described in further detail below. In some instances, the anti-TIGIT antagonist antibody has a VH domain having the amino acid sequence of SEQ ID NO: 17 and a VL domain having the amino acid sequence of SEQ ID NO: 19. In some instances, the anti-TIGIT antagonist antibody has a VH domain having the amino acid sequence of SEQ ID NO: 18 and a VL domain having the amino acid sequence of SEQ ID NO: 19.

In another aspect, the invention provides an anti-TIGIT antagonist antibody (e.g., an anti-TIGIT antagonist antibody disclosed herein, e.g., tiragolumab) and PD-1 axis binding antagonist (e.g., anti-PD-1 antagonist antibody (e.g., nivolumab)) for use in a method of treating a subject or population of subjects having a cancer with a detectable expression level of PD-L1 (e.g., cervical cancer, e.g., Stage IVB, metastatic, recurrent, or persistent cervical cancer, e.g., a metastatic and/or recurrent PD-L1-positive cervical carcinoma), wherein the method comprises administering to the subject or population of subjects one or more dosing cycles of an anti-TIGIT antagonist antibody at a dose (e.g., a fixed dose) of 600 mg every three weeks and nivolumab at a dose (e.g., a fixed dose) of 240 mg every two weeks, wherein the anti-TIGIT antagonist antibody comprises: a VH domain comprising the amino acid sequence of SEQ ID NO: 17 or 18; and a VL domain comprising the amino acid sequence of SEQ ID NO: 19, as described in further detail below. In some instances, the anti-TIGIT antagonist antibody has a VH domain having the amino acid sequence of SEQ ID NO: 17 and a VL domain having the amino acid sequence of SEQ ID NO: 19. In some instances, the anti-TIGIT antagonist antibody has a VH domain having the amino acid sequence of SEQ ID NO: 18 and a VL domain having the amino acid sequence of SEQ ID NO: 19.

In another aspect, the invention provides an anti-TIGIT antagonist antibody (e.g., an anti-TIGIT antagonist antibody disclosed herein, e.g., tiragolumab) and PD-1 axis binding antagonist (e.g., anti-PD-1 antagonist antibody (e.g., nivolumab)) for use in a method of treating a subject or population of subjects having a cancer with a detectable expression level of PD-L1 (e.g., cervical cancer, e.g., Stage IVB, metastatic, recurrent, or persistent cervical cancer, e.g., a metastatic and/or recurrent PD-L1-positive cervical carcinoma), wherein the method comprises administering to the subject or population of subjects one or more dosing cycles of an anti-TIGIT antagonist antibody at a dose (e.g., a fixed dose) of 600 mg every three weeks and nivolumab at a dose (e.g., a fixed dose) of 480 mg every four weeks, wherein the anti-TIGIT antagonist antibody comprises: a VH domain comprising the amino acid sequence of SEQ ID NO: 17 or 18; and a VL domain comprising the amino acid sequence of SEQ ID NO: 19, as described in further detail below. In some instances, the anti-TIGIT antagonist antibody has a VH domain having the amino acid sequence of SEQ ID NO: 17 and a VL domain having the amino acid sequence of SEQ ID NO: 19. In some instances, the anti-TIGIT antagonist antibody has a VH domain having the amino acid sequence of SEQ ID NO: 18 and a VL domain having the amino acid sequence of SEQ ID NO: 19.

In another aspect, the invention provides an anti-TIGIT antagonist antibody (e.g., an anti-TIGIT antagonist antibody disclosed herein, e.g., tiragolumab) and PD-1 axis binding antagonist (e.g., anti-PD-L1 antagonist antibody (e.g., atezolizumab)) for use in a method of treating a subject or population of subjects having a cancer with a detectable expression level of PD-L1 (e.g., cervical cancer, e.g., Stage IVB, metastatic, recurrent, or persistent cervical cancer, e.g., a metastatic and/or recurrent PD-L1-positive cervical carcinoma), wherein the method comprises administering to the subject or population of subjects one or more dosing cycles of tiragolumab at a dose (e.g., a fixed dose) of 600 mg every three weeks and atezolizumab at a dose (e.g., a fixed dose) of 1200 mg every three weeks.

In another aspect, the invention provides an anti-TIGIT antagonist antibody (e.g., an anti-TIGIT antagonist antibody disclosed herein, e.g., tiragolumab) and PD-1 axis binding antagonist (e.g., anti-PD-1 antagonist antibody (e.g., MK-3475 (pembrolizumab, previously known as lambrolizumab)) for use in a method of treating a subject or population of subjects having a cancer with a detectable expression level of PD-L1 (e.g., cervical cancer, e.g., Stage IVB, metastatic, recurrent, or persistent cervical cancer, e.g., a metastatic and/or recurrent PD-L1-positive cervical carcinoma), wherein the method comprises administering to the subject or population of subjects one or more dosing cycles of tiragolumab at a dose (e.g., a fixed dose) of 600 mg every three weeks and pembrolizumab at a dose (e.g., a fixed dose) of 200 mg every three weeks.

In another aspect, the invention provides an anti-TIGIT antagonist antibody (e.g., an anti-TIGIT antagonist antibody disclosed herein, e.g., tiragolumab) and PD-1 axis binding antagonist (e.g., anti-PD-1 antagonist antibody (e.g., MDX-1106 (nivolumab)) for use in a method of treating a subject or population of subjects having a cancer with a detectable expression level of PD-L1 (e.g., cervical cancer, e.g., Stage IVB, metastatic, recurrent, or persistent cervical cancer, e.g., a metastatic and/or recurrent PD-L1-positive cervical carcinoma), wherein the method comprises administering to the subject or population of subjects one or more dosing cycles of tiragolumab at a dose (e.g., a fixed dose) of 600 mg every three weeks and nivolumab at a dose (e.g., a fixed dose) of 240 mg every two weeks.

In another aspect, the invention provides an anti-TIGIT antagonist antibody (e.g., an anti-TIGIT antagonist antibody disclosed herein, e.g., tiragolumab) and PD-1 axis binding antagonist (e.g., anti-PD-1 antagonist antibody (e.g., MDX-1106 (nivolumab)) for use in a method of treating a subject or population of subjects having a cancer with a detectable expression level of PD-L1 (e.g., cervical cancer, e.g., Stage IVB, metastatic, recurrent, or persistent cervical cancer, e.g., a metastatic and/or recurrent PD-L1-positive cervical carcinoma), wherein the method comprises administering to the subject or population of subjects one or more dosing cycles of tiragolumab at a dose (e.g., a fixed dose) of 600 mg every three weeks and nivolumab at a dose (e.g., a fixed dose) of 480 mg every four weeks.

In another aspect, the invention provides uses of an anti-TIGIT antagonist antibody (e.g., an anti-TIGIT antagonist antibody disclosed herein, e.g., tiragolumab) and PD-1 axis binding antagonist (e.g., anti-PD-L1 antagonist antibody (e.g., atezolizumab) or anti-PD-1 antagonist antibody (e.g., MDX-1106 (nivolumab) or MK-3475 (pembrolizumab, previously known as lambrolizumab))) in the manufacture or preparation of a medicament for use in a method of treating a subject or population of subjects having a cancer with a detectable expression level of PD-L1 (e.g., cervical cancer, e.g., Stage IVB, metastatic, recurrent, or persistent cervical cancer, e.g., a metastatic and/or recurrent PD-L1-positive cervical carcinoma), wherein the method comprises administering to the subject or population of subjects one or more dosing cycles of the medicament, and wherein the medicament is formulated for administration of an effective amount of the anti-TIGIT antagonist antibody and an effective amount of the PD-1 axis binding antagonist (e.g., anti-PD-L1 antagonist antibody (e.g., atezolizumab) or anti-PD-1 antagonist antibody (e.g., MDX-1106 (nivolumab) or MK-3475 (pembrolizumab, previously known as lambrolizumab))).

In another aspect, the invention provides uses of an anti-TIGIT antagonist antibody in the manufacture of a medicament for use in a method of treating a subject or population of subjects having a cancer with a detectable expression level of PD-L1 (e.g., cervical cancer, e.g., Stage IVB, metastatic, recurrent, or persistent cervical cancer, e.g., a metastatic and/or recurrent PD-L1-positive cervical carcinoma), wherein the method comprises administering to the subject or population of subjects one or more dosing cycles of the medicament and a PD-1 axis binding antagonist (e.g., an anti-PD-L1 antagonist antibody (e.g., atezolizumab) or an anti-PD-1 antagonist antibody (e.g., MDX-1106 (nivolumab) or MK-3475 (pembrolizumab, previously known as lambrolizumab))), and wherein the medicament is formulated for administration of an effective amount of the anti-TIGIT antagonist antibody and an effective amount of the PD-1 axis binding antagonist (e.g., anti-PD-L1 antagonist antibody (e.g., atezolizumab) or anti-PD-1 antagonist antibody (e.g., MDX-1106 (nivolumab) or MK-3475 (pembrolizumab, previously known as lambrolizumab))).

In another aspect, the invention provides uses of a PD-1 axis binding antagonist (e.g., an anti-PD-L1 antagonist antibody (e.g., atezolizumab) or an anti-PD-1 antagonist antibody (e.g., MDX-1106 (nivolumab) or MK-3475 (pembrolizumab, previously known as lambrolizumab))) in the manufacture of a medicament for use in a method of treating a subject or population of subjects having a cancer with a detectable expression level of PD-L1 (e.g., cervical cancer, e.g., Stage IVB, metastatic, recurrent, or persistent cervical cancer, e.g., a metastatic and/or recurrent PD-L1-positive cervical carcinoma), wherein the method comprises administering to the subject or population of subjects one or more dosing cycles of the medicament and an anti-TIGIT antagonist antibody, and wherein the medicament is formulated for administration an effective amount of the PD-1 axis binding antagonist (e.g., anti-PD-L1 antagonist antibody (e.g., atezolizumab) or anti-PD-1 antagonist antibody (e.g., MDX-1106 (nivolumab) or MK-3475 (pembrolizumab, previously known as lambrolizumab))) and an effective amount of the anti-TIGIT antagonist antibody is to be administered.

In some instances, the effective amount of the anti-TIGIT antagonist antibody (e.g., an anti-TIGIT antagonist antibody as disclosed herein, e.g., tiragolumab) is a dose (e.g., a fixed dose) of between about 30 mg to about 1200 mg (e.g., between about 30 mg to about 1100 mg, e.g., between about 60 mg to about 1000 mg, e.g., between about 100 mg to about 900 mg, e.g., between about 200 mg to about 800 mg, e.g., between about 300 mg to about 800 mg, e.g., between about 400 mg to about 800 mg, e.g., between about 400 mg to about 750 mg, e.g., between about 450 mg to about 750 mg, e.g., between about 500 mg to about 700 mg, e.g., between about 550 mg to about 650 mg, e.g., 600 mg±10 mg, e.g., 600±6 mg, e.g., 600±5 mg, e.g., 600±3 mg, e.g., 600±1 mg, e.g., 600±0.5 mg, e.g., 600 mg) every three weeks. In some instances, an effective amount of the anti-TIGIT antagonist antibody (e.g., an anti-TIGIT antagonist antibody as disclosed herein, e.g., tiragolumab) is a dose of between about 30 mg to about 600 mg (e.g., between about 50 mg to between 600 mg, e.g., between about 60 mg to about 600 mg, e.g., between about 100 mg to about 600 mg, e.g., between about 200 mg to about 600 mg, e.g., between about 200 mg to about 550 mg, e.g., between about 250 mg to about 500 mg, e.g., between about 300 mg to about 450 mg, e.g., between about 350 mg to about 400 mg, e.g., about 375 mg) (e.g., between 30 mg to 1200 mg (e.g., between 30 mg to 1100 mg, e.g., between 60 mg to 1000 mg, e.g., between 100 mg to 900 mg, e.g., between 200 mg to 800 mg, e.g., between 300 mg to 800 mg, e.g., between 400 mg to 800 mg, e.g., between 400 mg to 750 mg, e.g., between 450 mg to 750 mg, e.g., between 500 mg to 700 mg, e.g., between 550 mg to 650 mg, e.g., 600 mg±10 mg, e.g., 600±6 mg, e.g., 600±5 mg, e.g., 600±3 mg, e.g., 600±1 mg, e.g., 600±0.5 mg, e.g., 600 mg) every three weeks. In some instances, an effective amount of the anti-TIGIT antagonist antibody (e.g., an anti-TIGIT antagonist antibody as disclosed herein, e.g., tiragolumab) is a dose of between 30 mg to 600 mg (e.g., between 50 mg to between 600 mg, e.g., between 60 mg to 600 mg, e.g., between 100 mg to 600 mg, e.g., between 200 mg to 600 mg, e.g., between 200 mg to 550 mg, e.g., between 250 mg to 500 mg, e.g., between 300 mg to 450 mg, e.g., between 350 mg to 400 mg, e.g., 375 mg)) every three weeks. In some instances, the effective amount of the anti-TIGIT antagonist antibody (e.g., an anti-TIGIT antagonist antibody as disclosed herein, e.g., tiragolumab) is a dose of about 600 mg every three weeks. In some instances, effective amount of the anti-TIGIT antagonist antibody (e.g., an anti-TIGIT antagonist antibody as disclosed herein, e.g., tiragolumab) is a dose of 600 mg every three weeks. In some instances, the dose of the anti-TIGIT antagonist antibody (e.g., an anti-TIGIT antagonist antibody as disclosed herein, e.g., tiragolumab) is to be administered in a combination therapy (e.g., a combination treatment with a PD-1 axis binding antagonist (e.g., an anti-PD-L1 antagonist antibody (e.g., atezolizumab) or an anti-PD-1 antagonist antibody (e.g., MDX-1106 (nivolumab) or MK-3475 (pembrolizumab, previously known as lambrolizumab)))) may be reduced as compared to a standard dose of the anti-TIGIT antagonist antibody is to be administered as a monotherapy.

In some instances, the effective amount of the PD-1 axis binding antagonist (e.g., anti-PD-L1 antagonist antibody (e.g., atezolizumab)) is a dose (e.g., a fixed dose) of between about 80 mg to about 2000 mg (e.g., between about 80 mg to about 1950 mg, e.g., between about 80 mg to about 1900 mg, e.g., between about 80 mg to about 1800 mg, e.g., between about 100 mg to about 1700 mg, e.g., between about 200 mg to about 1600 mg, e.g., between about 300 mg to about 1400 mg, e.g., between about 400 mg to about 1300 mg, e.g., between about 500 mg to about 1200 mg, e.g., between about 600 mg to about 1100 mg, e.g., between about 700 mg to about 1000 mg, e.g., between about 740 mg to about 940 mg, e.g., between about 790 mg to about 890 mg, e.g., between about 815 mg to about 865 mg, e.g., between about 830 mg to about 850 mg, e.g., 840 mg±5 mg, e.g., 840±2.5 mg, e.g., 840±1.0 mg, e.g., 840±0.5 mg, e.g., 840 mg) (e.g., between 80 mg to 2000 mg (e.g., between 80 mg to 1950 mg, e.g., between 80 mg to 1900 mg, e.g., between 80 mg to 1800 mg, e.g., between 100 mg to 1700 mg, e.g., between 200 mg to 1600 mg, e.g., between 300 mg to 1400 mg, e.g., between 400 mg to 1300 mg, e.g., between 500 mg to 1200 mg, e.g., between 600 mg to 1100 mg, e.g., between 700 mg to 1000 mg, e.g., between 740 mg to 940 mg, e.g., between 790 mg to 890 mg, e.g., between 815 mg to 865 mg, e.g., between 830 mg to 850 mg, e.g., 840 mg±5 mg, e.g., 840±2.5 mg, e.g., 840±1.0 mg, e.g., 840+0.5 mg, e.g., 840 mg)) every two weeks. In some instances, the effective amount of the PD-1 axis binding antagonist (e.g., anti-PD-L1 antagonist antibody (e.g., atezolizumab)) is a dose of between about 80 mg to about 2000 mg (e.g., between about 100 mg to about 2000 mg, e.g., between about 200 mg to about 1900 mg, e.g., between about 300 mg to about 1700 mg, e.g., between about 400 mg to about 1600 mg, e.g., between about 500 mg to about 1600 mg, e.g., between about 600 mg to about 1600 mg, e.g., between about 700 mg to about 1600 mg, e.g., between about 800 mg to about 1600 mg, e.g., between about 900 mg to about 1500 mg, e.g., between about 1000 mg to about 1400 mg, e.g., between about 1050 mg to about 1350 mg, e.g., between about 1100 mg to about 1300 mg, e.g., between about 1150 mg to about 1250 mg, e.g., between about 1175 mg to about 1225 mg (e.g., between 100 mg to 2000 mg, e.g., between 200 mg to 1900 mg, e.g., between 300 mg to 1700 mg, e.g., between 400 mg to 1600 mg, e.g., between 500 mg to 1600 mg, e.g., between 600 mg to 1600 mg, e.g., between 700 mg to 1600 mg, e.g., between 800 mg to 1600 mg, e.g., between 900 mg to 1500 mg, e.g., between 1000 mg to 1400 mg, e.g., between 1050 mg to 1350 mg, e.g., between 1100 mg to 1300 mg, e.g., between 1150 mg to 1250 mg, e.g., between 1175 mg to 1225 mg), e.g., between about 1190 mg to about 1210 mg (e.g., between 1190 mg to 1210 mg), e.g., 1200 mg±5 mg, e.g., 1200±2.5 mg, e.g., 1200±1.0 mg, e.g., 1200±0.5 mg, e.g., 1200 mg) every three weeks. In some instances, the effective amount of the PD-1 axis binding antagonist (e.g., anti-PD-L1 antagonist antibody (e.g., atezolizumab)) is a dose of between about 80 mg to about 2000 mg (e.g., between about 100 mg to about 2000 mg, e.g., between about 200 mg to about 2000 mg, e.g., between about 300 mg to about 2000 mg, e.g., between about 400 mg to about 2000 mg, e.g., between about 500 mg to about 2000 mg, e.g., between about 600 mg to about 1900 mg, e.g., between about 700 mg to about 1800 mg, e.g., between about 800 mg to about 1800 mg, e.g., between about 900 mg to about 1800 mg, e.g., between about 1000 mg to about 1800 mg, e.g., between about 1100 mg to about 1800 mg, e.g., between about 1200 mg to about 1800 mg, e.g., between about 1300 mg to about 1800 mg, e.g., between about 1400 mg to about 1800 mg, e.g., between about 1500 mg to about 1800 mg, e.g., between about 1580 mg to about 1780 mg, e.g., between about 1630 mg to about 1730 mg, e.g., between about 1655 mg to about 1705 mg, e.g., between about 1670 mg to about 1690 mg (e.g., between 100 mg to 2000 mg, e.g., between 200 mg to 2000 mg, e.g., between 300 mg to 2000 mg, e.g., between 400 mg to 2000 mg, e.g., between 500 mg to 2000 mg, e.g., between 600 mg to 1900 mg, e.g., between 700 mg to 1800 mg, e.g., between 800 mg to 1800 mg, e.g., between 900 mg to 1800 mg, e.g., between 1000 mg to 1800 mg, e.g., between 1100 mg to 1800 mg, e.g., between 1200 mg to 1800 mg, e.g., between 1300 mg to 1800 mg, e.g., between 1400 mg to 1800 mg, e.g., between 1500 mg to 1800 mg, e.g., between 1580 mg to 1780 mg, e.g., between 1630 mg to 1730 mg, e.g., between 1655 mg to 1705 mg, e.g., between 1670 mg to 1690 mg), e.g., 1680 mg±5 mg, e.g., 1680±2.5 mg, e.g., 1680±1.0 mg, e.g., 1680±0.5 mg, e.g., 1680 mg) every four weeks. In some instances, the effective amount of the PD-1 axis binding antagonist (e.g., anti-PD-L1 antagonist antibody (e.g., atezolizumab)) is a dose of about 840 mg every two weeks. In some instances, the effective amount of the PD-1 axis binding antagonist (e.g., anti-PD-L1 antagonist antibody (e.g., atezolizumab)) is a dose of 840 mg every two weeks. In some instances, the effective amount of the PD-1 axis binding antagonist (e.g., anti-PD-L1 antagonist antibody (e.g., atezolizumab)) is a dose of about 1200 mg every three weeks. In some instances, the effective amount of the PD-1 axis binding antagonist (e.g., anti-PD-L1 antagonist antibody (e.g., atezolizumab)) is a dose of 1200 mg every three weeks. In some instances, the effective amount of the PD-1 axis binding antagonist (e.g., anti-PD-L1 antagonist antibody (e.g., atezolizumab)) is a dose of about 1680 mg every four weeks. In some instances, the effective amount of the PD-1 axis binding antagonist (e.g., anti-PD-L1 antagonist antibody (e.g., atezolizumab)) is a dose of 1680 mg every four weeks. In some instances, the dose of the PD-1 axis binding antagonist (e.g., an anti-PD-L1 antagonist antibody (e.g., atezolizumab) or an anti-PD-1 antagonist antibody (e.g., MDX-1106 (nivolumab) or MK-3475 (pembrolizumab, previously known as lambrolizumab))) to be administered in a combination therapy (e.g., a combination treatment with an anti-TIGIT antagonist antibody, such as an anti-TIGIT antagonist antibody disclosed herein, e.g., tiragolumab) may be reduced as compared to a standard dose of the anti-PD-L1 antagonist antibody to be administered as a monotherapy.

In some instances, the effective amount of the PD-1 axis binding antagonist (e.g., anti-PD-1 antagonist antibody (e.g., pembrolizumab)) is a dose (e.g., a fixed dose) of between about 20 mg to about 1000 mg (e.g., between about 40 mg to about 900 mg, e.g., between about 60 mg to about 800 mg, e.g., between about 80 mg to about 700 mg, e.g., between about 80 mg to about 600 mg, e.g., between about 100 mg to about 500 mg, e.g., between about 120 mg to about 400 mg, e.g., between about 140 mg to about 300 mg, e.g., between about 160 mg to about 350 mg, e.g., between about 180 mg to about 300 mg, e.g., between about 180 mg to about 250 mg, e.g., between about 180 mg to about 220 mg, e.g., between about 190 mg to about 210 mg (e.g., between 40 mg to 900 mg, e.g., between 60 mg to 800 mg, e.g., between 80 mg to 700 mg, e.g., between 80 mg to 600 mg, e.g., between 100 mg to 500 mg, e.g., between 120 mg to 400 mg, e.g., between 140 mg to 300 mg, e.g., between 160 mg to 350 mg, e.g., between 180 mg to 300 mg, e.g., between 180 mg to 250 mg, e.g., between 180 mg to 220 mg, e.g., between 190 mg to 210 mg), e.g., 200 mg±5 mg, e.g., 200±2.5 mg, e.g., 200±1.0 mg, e.g., 200±0.5 mg, e.g., 200 mg) every three weeks. In some instances, the effective amount of the PD-1 axis binding antagonist (e.g., anti-PD-1 antagonist antibody (e.g., pembrolizumab)) is a dose of about 200 mg every three weeks. In some instances, the effective amount of the PD-1 axis binding antagonist (e.g., anti-PD-1 antagonist antibody (e.g., pembrolizumab)) is a dose of 200 mg every three weeks. In some instances, the dose of the PD-1 axis binding antagonist (e.g., anti-PD-1 antagonist antibody (e.g., pembrolizumab)) administered in a combination therapy (e.g., a combination treatment with an anti-TIGIT antagonist antibody, such as an anti-TIGIT antagonist antibody disclosed herein, e.g., tiragolumab) may be reduced as compared to a standard dose of the anti-PD-L1 antagonist antibody administered as a monotherapy.

In some instances, the effective amount of the PD-1 axis binding antagonist (e.g., anti-PD-1 antagonist antibody (e.g., nivolumab)) is a dose (e.g., a fixed dose) of between about 20 mg to about 1000 mg (e.g., 20 mg to 1000 mg) (e.g., between about 40 mg to about 900 mg, e.g., between about 60 mg to about 800 mg, e.g., between about 80 mg to about 700 mg, e.g., between about 80 mg to about 600 mg, e.g., between about 100 mg to about 500 mg, e.g., between about 120 mg to about 400 mg, e.g., between about 140 mg to about 300 mg, e.g., between about 160 mg to about 350 mg, e.g., between about 180 mg to about 300 mg, e.g., between about 200 mg to about 280 mg, e.g., between about 220 mg to about 260 mg, e.g., between about 230 mg to about 250 mg (e.g., between 40 mg to 900 mg, e.g., between 60 mg to 800 mg, e.g., between 80 mg to 700 mg, e.g., between 80 mg to 600 mg, e.g., between 100 mg to 500 mg, e.g., between 120 mg to 400 mg, e.g., between 140 mg to 300 mg, e.g., between 160 mg to 350 mg, e.g., between 180 mg to 300 mg, e.g., between 200 mg to 280 mg, e.g., between 220 mg to 260 mg, e.g., between 230 mg to 250 mg), e.g., 240 mg±5 mg, e.g., 240±2.5 mg, e.g., 240±1.0 mg, e.g., 240±0.5 mg, e.g., 240 mg) every two weeks. In some instances, the effective amount of the PD-1 axis binding antagonist (e.g., anti-PD-1 antagonist antibody (e.g., nivolumab)) is a dose of about 240 mg every two weeks. In some instances, the effective amount of the PD-1 axis binding antagonist (e.g., anti-PD-1 antagonist antibody (e.g., nivolumab)) is a dose of 240 mg every two weeks. In some instances, the effective amount of the PD-1 axis binding antagonist (e.g., anti-PD-1 antagonist antibody (e.g., nivolumab)) is a dose of between about 100 mg to about 1000 mg (e.g., between 100 mg to 1000 mg) (e.g., between about 200 mg to about 900 mg, e.g., between about 300 mg to about 800 mg, e.g., between about 400 mg to about 700 mg, e.g., between about 400 mg to about 600 mg, e.g., between about 400 mg to about 550 mg, e.g., between about 420 mg to about 540 mg, e.g., between about 440 mg to about 520 mg, e.g., between about 460 mg to about 500 mg, e.g., between about 470 mg to about 490 mg (e.g., between 200 mg to 900 mg, e.g., between 300 mg to 800 mg, e.g., between 400 mg to 700 mg, e.g., between 400 mg to 600 mg, e.g., between 400 mg to 550 mg, e.g., between 420 mg to 540 mg, e.g., between 440 mg to 520 mg, e.g., between 460 mg to 500 mg, e.g., between 470 mg to 490 mg), e.g., 480 mg±5 mg, e.g., 480±2.5 mg, e.g., 480±1.0 mg, e.g., 480±0.5 mg, e.g., 480 mg) every four weeks. In some instances, the effective amount of the PD-1 axis binding antagonist (e.g., anti-PD-1 antagonist antibody (e.g., nivolumab)) is a dose of about 480 mg every four weeks. In some instances, the effective amount of the PD-1 axis binding antagonist (e.g., anti-PD-1 antagonist antibody (e.g., nivolumab)) is a dose of 480 mg every four weeks. In some instances, the dose of the PD-1 axis binding antagonist (e.g., anti-PD-1 antagonist antibody (e.g., nivolumab)) administered in a combination therapy (e.g., a combination treatment with an anti-TIGIT antagonist antibody, such as an anti-TIGIT antagonist antibody disclosed herein, e.g., tiragolumab) may be reduced as compared to a standard dose of the anti-PD-L1 antagonist antibody administered as a monotherapy.

In some instances, the effective amount of the PD-1 axis binding antagonist (e.g., anti-PD-L1 antagonist antibody (e.g., atezolizumab)) is a dose of between about 0.01 mg/kg to about 50 mg/kg of the subject's body weight (e.g., between about 0.01 mg/kg to about 45 mg/kg, e.g., between about 0.1 mg/kg to about 40 mg/kg, e.g., between about 1 mg/kg to about 35 mg/kg, e.g., between about 2.5 mg/kg to about 30 mg/kg, e.g., between about 5 mg/kg to about 25 mg/kg, e.g., between about 10 mg/kg to about 20 mg/kg, e.g., between about 12.5 mg/kg to about 15 mg/kg, e.g., about 15±2 mg/kg, about 15±1 mg/kg, about 15±0.5 mg/kg, about 15±0.2 mg/kg, or about 15±0.1 mg/kg, e.g., about 15 mg/kg) every three weeks. In some instances, the effective amount of the PD-1 axis binding antagonist (e.g., anti-PD-L1 antagonist antibody (e.g., atezolizumab)) is a dose of between about 0.01 mg/kg to about 15 mg/kg of the subject's body weight (e.g., between about 0.1 mg/kg to about 15 mg/kg, e.g., between about 0.5 mg/kg to about 15 mg/kg, e.g., between about 1 mg/kg to about 15 mg/kg, e.g., between about 2.5 mg/kg to about 15 mg/kg, e.g., between about 5 mg/kg to about 15 mg/kg, e.g., between about 7.5 mg/kg to about 15 mg/kg, e.g., between about 10 mg/kg to about 15 mg/kg, e.g., between about 12.5 mg/kg to about 15 mg/kg, e.g., between about 14 mg/kg to about 15 mg/kg, e.g., about 15±1 mg/kg, e.g., about 15±0.5 mg/kg, e.g., about 15±0.2 mg/kg, e.g., about 15±0.1 mg/kg, e.g., about 15 mg/kg) every three weeks. In some instances, the effective amount of anti-PD-L1 antagonist antibody (e.g., atezolizumab) is a dose of about 15 mg/kg to be administered every three weeks. In some instances, the dose of the PD-1 axis binding antagonist (e.g., an anti-PD-L1 antagonist antibody (e.g., atezolizumab) or an anti-PD-1 antagonist antibody (e.g., MDX-1106 (nivolumab) or MK-3475 (pembrolizumab, previously known as lambrolizumab))) administered in a combination therapy (e.g., a combination treatment with an anti-TIGIT antagonist antibody, such as an anti-TIGIT antagonist antibody disclosed herein, e.g., tiragolumab) may be reduced as compared to a standard dose of the anti-PD-L1 antagonist antibody administered as a monotherapy.

In any of the uses of the invention, the medicament comprising the anti-TIGIT antagonist antibody (e.g., an anti-TIGIT antagonist antibody as disclosed herein, e.g., tiragolumab) and the PD-1 axis binding antagonist (e.g., an anti-PD-L1 antagonist antibody (e.g., atezolizumab) or an anti-PD-1 antagonist antibody (e.g., MDX-1106 (nivolumab) or MK-3475 (pembrolizumab, previously known as lambrolizumab))) may be administered in one or more dosing cycles (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 or more dosing cycles). In some instances, the dosing cycles of the medicament comprising anti-TIGIT antagonist antibody (e.g., an anti-TIGIT antagonist antibody as disclosed herein, e.g., tiragolumab) and the PD-1 axis binding antagonist (e.g., an anti-PD-L1 antagonist antibody (e.g., atezolizumab) or an anti-PD-1 antagonist antibody (e.g., MDX-1106 (nivolumab) or MK-3475 (pembrolizumab, previously known as lambrolizumab))) continue until there is a loss of clinical benefit (e.g., confirmed disease progression, drug resistance, death, or unacceptable toxicity). In some instances, the length of each dosing cycle is about 14 to 28 days (e.g., 14 days, 15 days, 16 days, 17 days, 18 days, 19 days, 20 days, 21 days, 22 days, 23 days, 24 days, 25 days, 26 days, 27 days, or 28 days). In some instances, the length of each dosing cycle is about 21 days. In some instances, the medicament comprising the anti-TIGIT antagonist antibody (e.g., an anti-TIGIT antagonist antibody as disclosed herein, e.g., tiragolumab) is to be administered on about Day 1 (e.g., Day 1±3 days) of each dosing cycle. For example, the medicament comprising the anti-TIGIT antagonist antibody (e.g., an anti-TIGIT antagonist antibody as disclosed herein, e.g., tiragolumab) is to be administered intravenously at a dose (e.g., a fixed dose) of about 600 mg on Day 1 of each 21-day cycle (i.e., at a dose of about 600 mg every three weeks). Similarly, in some instances, the medicament comprising the PD-1 axis binding antagonist (e.g., an anti-PD-L1 antagonist antibody (e.g., atezolizumab) or an anti-PD-1 antagonist antibody (e.g., MDX-1106 (nivolumab) or MK-3475 (pembrolizumab, previously known as lambrolizumab))) is to be administered on about Day 1 (e.g., Day 1±3 days) of each dosing cycle. For example, the medicament comprising the PD-1 axis binding antagonist (e.g., anti-PD-L1 antagonist antibody (e.g., atezolizumab)) is to be administered intravenously at a dose of about 1200 mg on Day 1 of each 21-day cycle (i.e., at a dose of about 1200 mg every three weeks). For example, the medicament comprising the PD-1 axis binding antagonist (e.g., anti-PD-1 antagonist antibody (e.g., pembrolizumab)) is to be administered intravenously at a dose of about 200 mg on Day 1 of each 21-day cycle (i.e., at a dose of about 200 mg every three weeks). For example, the medicament comprising the PD-1 axis binding antagonist (e.g., anti-PD-1 antagonist antibody (e.g., nivolumab)) is to be administered intravenously at a dose of about 240 mg on Day 1 of each 14-day cycle (i.e., at a dose of about 240 mg every two weeks). For example, the medicament comprising the PD-1 axis binding antagonist (e.g., anti-PD-1 antagonist antibody (e.g., nivolumab)) is to be administered intravenously at a dose of about 480 mg on Day 1 of each 28-day cycle (i.e., at a dose of about 480 mg every four weeks). In some instances, the medicament comprising both the anti-TIGIT antagonist antibody (e.g., an anti-TIGIT antagonist antibody as disclosed herein, e.g., tiragolumab) and the PD-1 axis binding antagonist (e.g., an anti-PD-L1 antagonist antibody (e.g., atezolizumab) or an anti-PD-1 antagonist antibody (e.g., MDX-1106 (nivolumab) or MK-3475 (pembrolizumab, previously known as lambrolizumab))) are to be administered on about Day 1 (e.g., Day 1±3 days) of each dosing cycle. For example, the medicament comprising the anti-TIGIT antagonist antibody (e.g., an anti-TIGIT antagonist antibody as disclosed herein, e.g., tiragolumab) is to be administered intravenously at a dose of about 600 mg on Day 1 of each 21-day cycle (i.e., at a dose of about 600 mg every three weeks), and the medicament comprising the PD-1 axis binding antagonist (e.g., anti-PD-L1 antagonist antibody (e.g., atezolizumab)) is to be administered intravenously at a dose of about 1200 mg on Day 1 of each 21-day cycle (i.e., at a dose of about 1200 mg every three weeks). For example, the medicament comprising the anti-TIGIT antagonist antibody (e.g., an anti-TIGIT antagonist antibody as disclosed herein, e.g., tiragolumab) is to be administered intravenously at a dose of about 600 mg on Day 1 of each 21-day cycle (i.e., at a dose of about 600 mg every three weeks), and the medicament comprising the PD-1 axis binding antagonist (e.g., anti-PD-1 antagonist antibody (e.g., pembrolizumab)) is to be administered intravenously at a dose of about 200 mg on Day 1 of each 21-day cycle (i.e., at a dose of about 200 mg every three weeks). For example, the medicament comprising the anti-TIGIT antagonist antibody (e.g., an anti-TIGIT antagonist antibody as disclosed herein, e.g., tiragolumab) is to be administered intravenously at a dose of about 600 mg on Day 1 of each 21-day cycle (i.e., at a dose of about 600 mg every three weeks), and the medicament comprising the PD-1 axis binding antagonist (e.g., anti-PD-1 antagonist antibody (e.g., nivolumab)) is to be administered intravenously at a dose of about 240 mg on Day 1 of each 14-day cycle (i.e., at a dose of about 240 mg every two weeks). For example, the medicament comprising the anti-TIGIT antagonist antibody (e.g., an anti-TIGIT antagonist antibody as disclosed herein, e.g., tiragolumab) is to be administered intravenously at a dose of about 600 mg on Day 1 of each 21-day cycle (i.e., at a dose of about 600 mg every three weeks), and the medicament comprising the PD-1 axis binding antagonist (e.g., anti-PD-1 antagonist antibody (e.g., nivolumab)) is to be administered intravenously at a dose of about 480 mg on Day 1 of each 28-day cycle (i.e., at a dose of about 480 mg every four weeks).

In some instances, the medicament comprising the anti-TIGIT antagonist antibody (e.g., an anti-TIGIT antagonist antibody as disclosed herein, e.g., tiragolumab) is administered to the subject or population of subjects by intravenous infusion over about 60±15 minutes (e.g., about 50 minutes, about 51 minutes, about 52 minutes, about 53 minutes, about 54 minutes, about 55 minutes, about 56 minutes, about 57 minutes, about 58 minutes, about 59 minutes, about 60 minutes, about 61 minutes, about 62 minutes, about 63 minutes, about 64 minutes, about 65 minutes, about 66 minutes, about 67 minutes, about 68 minutes, about 69 minutes, or about 70 minutes). In some instances, the medicament comprising the PD-1 axis binding antagonist (e.g., an anti-PD-L1 antagonist antibody (e.g., atezolizumab) or an anti-PD-1 antagonist antibody (e.g., MDX-1106 (nivolumab) or MK-3475 (pembrolizumab, previously known as lambrolizumab))) is to be administered to the subject or population of subjects by intravenous infusion over about 60±15 minutes (e.g., about 45 minutes, about 46 minutes, about 47 minutes, about 48 minutes, about 49 minutes, about 50 minutes, about 51 minutes, about 52 minutes, about 53 minutes, about 54 minutes, about 55 minutes, about 56 minutes, about 57 minutes, about 58 minutes, about 59 minutes, about 60 minutes, about 61 minutes, about 62 minutes, about 63 minutes, about 64 minutes, about 65 minutes, about 66 minutes, about 67 minutes, about 68 minutes, about 69 minutes, about 70 minutes, about 71 minutes, about 72 minutes, about 73 minutes, about 74 minutes, or about 75 minutes).

In some instances, the medicament comprising the anti-TIGIT antagonist antibody (e.g., an anti-TIGIT antagonist antibody as disclosed herein, e.g., tiragolumab) is administered to the subject or population of subjects by intravenous infusion over about 30±10 minutes (e.g., about 20 minutes, about 21 minutes, about 22 minutes, about 23 minutes, about 24 minutes, about 25 minutes, about 26 minutes, about 27 minutes, about 28 minutes, about 29 minutes, about 30 minutes, about 31 minutes, about 32 minutes, about 33 minutes, about 34 minutes, about 35 minutes, about 36 minutes, about 37 minutes, about 38 minutes, about 39 minutes, or about 40 minutes). In some instances, the medicament comprising the PD-1 axis binding antagonist (e.g., an anti-PD-L1 antagonist antibody (e.g., atezolizumab) or an anti-PD-1 antagonist antibody (e.g., MDX-1106 (nivolumab) or MK-3475 (pembrolizumab, previously known as lambrolizumab))) is to be administered to the subject or population of subjects by intravenous infusion over about 30±10 minutes (e.g., about 20 minutes, about 21 minutes, about 22 minutes, about 23 minutes, about 24 minutes, about 25 minutes, about 26 minutes, about 27 minutes, about 28 minutes, about 29 minutes, about 30 minutes, about 31 minutes, about 32 minutes, about 33 minutes, about 34 minutes, about 35 minutes, about 36 minutes, about 37 minutes, about 38 minutes, about 39 minutes, or about 40 minutes).

In some instances, the medicament comprising the anti-TIGIT antagonist antibody (e.g., an anti-TIGIT antagonist antibody as disclosed herein, e.g., tiragolumab) is to be administered to the subject or population of subjects before the medicament comprising the PD-1 axis binding antagonist (e.g., an anti-PD-L1 antagonist antibody (e.g., atezolizumab) or an anti-PD-1 antagonist antibody (e.g., MDX-1106 (nivolumab) or MK-3475 (pembrolizumab, previously known as lambrolizumab))). In some instances, for example, following administration of the medicament comprising the anti-TIGIT antagonist antibody and before administration of the medicament comprising the anti-PD-L1 antagonist antibody, the method includes an intervening first observation period. In some instances, the method further includes a second observation period following administration of the anti-PD-L1 antagonist antibody. In some instances, the method includes both a first observation period following administration of the medicament comprising the anti-TIGIT antagonist antibody and second observation period following administration of the medicament comprising the anti-PD-L1 antagonist antibody. In some instances, the first and second observation periods are each between about 30 minutes to about 60 minutes in length. In instances in which the first and second observation periods are each about 60 minutes in length, the method may include recording the subject's vital signs (e.g., pulse rate, respiratory rate, blood pressure, and temperature) at about 30±10 minutes after administration of the medicament comprising the anti-TIGIT antagonist antibody and the medicament comprising the anti-PD-L1 antagonist antibody during the first and second observation periods, respectively. In instances in which the first and second observation periods are each about 30 minutes in length, the method may include recording the subject's vital signs (e.g., pulse rate, respiratory rate, blood pressure, and temperature) at about 15±10 minutes after administration of the medicament comprising the anti-TIGIT antagonist antibody and the medicament comprising the anti-PD-L1 antagonist antibody during the first and second observation periods, respectively.

In other instances, the medicament comprising the PD-1 axis binding antagonist (e.g., an anti-PD-L1 antagonist antibody (e.g., atezolizumab) or an anti-PD-1 antagonist antibody (e.g., MDX-1106 (nivolumab) or MK-3475 (pembrolizumab, previously known as lambrolizumab))) is to be administered to the subject or population of subjects before the anti-TIGIT antagonist antibody (e.g., an anti-TIGIT antagonist antibody as disclosed herein, e.g., tiragolumab). In some instances, for example, following administration of the medicament comprising the anti-PD-L1 antagonist antibody and before administration of the medicament comprising the anti-TIGIT antagonist antibody, the method includes an intervening first observation period. In some instances, the method includes a second observation period following administration of the medicament comprising the anti-TIGIT antagonist antibody. In some instances, the method includes both a first observation period following administration of the medicament comprising the anti-PD-L1 antagonist antibody and second observation period following administration of the medicament comprising the anti-TIGIT antagonist antibody. In some instances, the first and second observation periods are each between about 30 minutes to about 60 minutes in length. In instances in which the first and second observation periods are each about 60 minutes in length, the method may include recording the subject's vital signs (e.g., pulse rate, respiratory rate, blood pressure, and temperature) at about 30±10 minutes after administration of the medicament comprising the anti-PD-L1 antagonist antibody and the medicament comprising the anti-TIGIT antagonist antibody during the first and second observation periods, respectively. In instances in which the first and second observation periods are each about 30 minutes in length, the method may include recording the subject's vital signs (e.g., pulse rate, respiratory rate, blood pressure, and temperature) at about 15±10 minutes after administration of the medicament comprising the anti-PD-L1 antagonist antibody and the medicament comprising the anti-TIGIT antagonist antibody during the first and second observation periods, respectively.

In other instances, the medicament comprising the anti-TIGIT antagonist antibody (e.g., an anti-TIGIT antagonist antibody as disclosed herein, e.g., tiragolumab) and the medicament comprising the PD-1 axis binding antagonist (e.g., an anti-PD-L1 antagonist antibody (e.g., atezolizumab) or an anti-PD-1 antagonist antibody (e.g., MDX-1106 (nivolumab) or MK-3475 (pembrolizumab, previously known as lambrolizumab))) is to be administered to the subject or population of subjects simultaneously. In some instances, for example, following administration of the medicament comprising the anti-TIGIT antagonist antibody and the medicament comprising the anti-PD-L1 antagonist antibody the method includes an observation period. In some instances, the observation period is between about 30 minutes to about 60 minutes in length. In instances in which the observation period is about 60 minutes in length, the method may include recording the subject's vital signs (e.g., pulse rate, respiratory rate, blood pressure, and temperature) at about 30±10 minutes after administration of the medicament comprising the anti-PD-L1 antagonist antibody and the medicament comprising the anti-TIGIT antagonist antibody during the observation period. In instances in which the observation period is about 30 minutes in length, the method may include recording the subject's vital signs (e.g., pulse rate, respiratory rate, blood pressure, and temperature) at about 15±10 minutes after administration of the medicament comprising the anti-PD-L1 antagonist antibody and the medicament comprising the anti-TIGIT antagonist antibody during the observation period.

In another aspect, the invention provides uses of an anti-TIGIT antagonist antibody (e.g., an anti-TIGIT antagonist antibody disclosed herein, e.g., tiragolumab) and anti-PD-L1 antagonist antibody (e.g., atezolizumab) in the manufacture or preparation of a medicament for use in a method of treating a subject or population of subjects having a cancer with a detectable expression level of PD-L1 (e.g., cervical cancer, e.g., Stage IVB, metastatic, recurrent, or persistent cervical cancer, e.g., a metastatic and/or recurrent PD-L1-positive cervical carcinoma), wherein the method comprises administering to the subject or population of subjects one or more dosing cycles of the medicament, and wherein the medicament is formulated for administration of the anti-TIGIT antagonist antibody at a dose (e.g., a fixed dose) of between about 30 mg to about 1200 mg (e.g., between 30 mg to 1200 mg) every three weeks and the PD-1 axis binding antagonist (e.g., an anti-PD-L1 antagonist antibody (e.g., atezolizumab) or an anti-PD-1 antagonist antibody (e.g., MDX-1106 (nivolumab) or MK-3475 (pembrolizumab, previously known as lambrolizumab))) at a dose (e.g., a fixed dose) of between about 80 mg to about 2000 mg (e.g., between 80 mg to 2000 mg) every three weeks.

In another aspect, the invention provides uses of an anti-TIGIT antagonist antibody (e.g., an anti-TIGIT antagonist antibody disclosed herein, e.g., tiragolumab) and anti-PD-L1 antagonist antibody (e.g., atezolizumab) in the manufacture or preparation of a medicament for use in a method of treating a subject or population of subjects having a cancer with a detectable expression level of PD-L1 (e.g., cervical cancer, e.g., Stage IVB, metastatic, recurrent, or persistent cervical cancer, e.g., a metastatic and/or recurrent PD-L1-positive cervical carcinoma), wherein the method comprises administering to the subject or population of subjects one or more dosing cycles of the medicament, and wherein the medicament is formulated for administration of the anti-TIGIT antagonist antibody at a dose (e.g., a fixed dose) of between about 30 mg to about 1200 mg (e.g., between 30 mg to 1200 mg) every three weeks and the PD-1 axis binding antagonist (e.g., an anti-PD-L1 antagonist antibody (e.g., atezolizumab) or an anti-PD-1 antagonist antibody (e.g., MDX-1106 (nivolumab) or MK-3475 (pembrolizumab, previously known as lambrolizumab))) at a dose (e.g., a fixed dose) of between about 80 mg to about 2000 mg (e.g., between 80 mg to 2000 mg) every two weeks.

In another aspect, the invention provides uses of an anti-TIGIT antagonist antibody (e.g., an anti-TIGIT antagonist antibody disclosed herein, e.g., tiragolumab) and anti-PD-L1 antagonist antibody (e.g., atezolizumab) in the manufacture or preparation of a medicament for use in a method of treating a subject or population of subjects having a cancer with a detectable expression level of PD-L1 (e.g., cervical cancer, e.g., Stage IVB, metastatic, recurrent, or persistent cervical cancer, e.g., a metastatic and/or recurrent PD-L1-positive cervical carcinoma), wherein the method comprises administering to the subject or population of subjects one or more dosing cycles of the medicament, and wherein the medicament is formulated for administration of the anti-TIGIT antagonist antibody at a dose (e.g., a fixed dose) of between about 30 mg to about 1200 mg every three weeks and the PD-1 axis binding antagonist (e.g., an anti-PD-L1 antagonist antibody (e.g., atezolizumab) or an anti-PD-1 antagonist antibody (e.g., MDX-1106 (nivolumab) or MK-3475 (pembrolizumab, previously known as lambrolizumab))) at a dose (e.g., a fixed dose) of between about 80 mg to about 2000 mg (e.g., between 80 mg to 2000 mg) every four weeks.

In another aspect, the invention provides uses of a PD-1 axis binding antagonist (e.g., an anti-PD-L1 antagonist antibody (e.g., atezolizumab) or an anti-PD-1 antagonist antibody (e.g., MDX-1106 (nivolumab) or MK-3475 (pembrolizumab, previously known as lambrolizumab))) in the manufacture of a medicament for use in a method of treating a subject or population of subjects having a cancer with a detectable expression level of PD-L1 (e.g., cervical cancer, e.g., Stage IVB, metastatic, recurrent, or persistent cervical cancer, e.g., a metastatic and/or recurrent PD-L1-positive cervical carcinoma), wherein the method comprises administering to the subject or population of subjects one or more dosing cycles of the medicament and an anti-TIGIT antagonist antibody, and wherein the medicament is formulated for administration of the PD-1 axis binding antagonist (e.g., an anti-PD-L1 antagonist antibody (e.g., atezolizumab) or an anti-PD-1 antagonist antibody (e.g., MDX-1106 (nivolumab) or MK-3475 (pembrolizumab, previously known as lambrolizumab))) at a dose (e.g., a fixed dose) of between about 80 mg to about 2000 mg (e.g., between 80 mg to 2000 mg) every three weeks and the anti-TIGIT antagonist antibody is to be administered at a dose (e.g., a fixed dose) of between about 30 mg to about 1200 mg (e.g., between 30 mg to 1200 mg) every three weeks.

In another aspect, the invention provides uses of a PD-1 axis binding antagonist (e.g., an anti-PD-L1 antagonist antibody (e.g., atezolizumab) or an anti-PD-1 antagonist antibody (e.g., MDX-1106 (nivolumab) or MK-3475 (pembrolizumab, previously known as lambrolizumab))) in the manufacture of a medicament for use in a method of treating a subject or population of subjects having a cancer with a detectable expression level of PD-L1 (e.g., cervical cancer, e.g., Stage IVB, metastatic, recurrent, or persistent cervical cancer, e.g., a metastatic and/or recurrent PD-L1-positive cervical carcinoma), wherein the method comprises administering to the subject or population of subjects one or more dosing cycles of the medicament and an anti-TIGIT antagonist antibody, and wherein the medicament is formulated for administration of the PD-1 axis binding antagonist (e.g., an anti-PD-L1 antagonist antibody (e.g., atezolizumab) or an anti-PD-1 antagonist antibody (e.g., MDX-1106 (nivolumab) or MK-3475 (pembrolizumab, previously known as lambrolizumab))) at a dose (e.g., a fixed dose) of between about 80 mg to about 2000 mg (e.g., between 80 mg to 2000 mg) every four weeks and the anti-TIGIT antagonist antibody is to be administered at a dose (e.g., a fixed dose) of between about 30 mg to about 1200 (e.g., between 30 mg to 1200 mg) mg every three weeks.

In another aspect, the invention provides uses of a PD-1 axis binding antagonist (e.g., an anti-PD-L1 antagonist antibody (e.g., atezolizumab) or an anti-PD-1 antagonist antibody (e.g., MDX-1106 (nivolumab) or MK-3475 (pembrolizumab, previously known as lambrolizumab))) in the manufacture of a medicament for use in a method of treating a subject or population of subjects having a cancer with a detectable expression level of PD-L1 (e.g., cervical cancer, e.g., Stage IVB, metastatic, recurrent, or persistent cervical cancer, e.g., a metastatic and/or recurrent PD-L1-positive cervical carcinoma), wherein the method comprises administering to the subject or population of subjects one or more dosing cycles of the medicament and an anti-TIGIT antagonist antibody, and wherein the medicament is formulated for administration of the PD-1 axis binding antagonist (e.g., an anti-PD-L1 antagonist antibody (e.g., atezolizumab) or an anti-PD-1 antagonist antibody (e.g., MDX-1106 (nivolumab) or MK-3475 (pembrolizumab, previously known as lambrolizumab))) at a dose (e.g., a fixed dose) of between about 80 mg to about 2000 mg (e.g., between 80 mg to 2000 mg) every two weeks and the anti-TIGIT antagonist antibody is to be administered at a dose (e.g., a fixed dose) of between about 30 mg to about 1200 mg (e.g., between 30 mg to 1200 mg) every three weeks.

In another aspect, the invention provides uses of an anti-TIGIT antagonist antibody in the manufacture of a medicament for use in a method of treating a subject or population of subjects having a cancer with a detectable expression level of PD-L1 (e.g., cervical cancer, e.g., Stage IVB, metastatic, recurrent, or persistent cervical cancer, e.g., a metastatic and/or recurrent PD-L1-positive cervical carcinoma), wherein the method comprises administering to the subject or population of subjects one or more dosing cycles of the medicament and a PD-1 axis binding antagonist (e.g., an anti-PD-L1 antagonist antibody (e.g., atezolizumab) or an anti-PD-1 antagonist antibody (e.g., MDX-1106 (nivolumab) or MK-3475 (pembrolizumab, previously known as lambrolizumab))), and wherein the medicament is formulated for administration of the anti-TIGIT antagonist antibody at a dose (e.g., a fixed dose) of between about 30 mg to about 1200 mg (e.g., between 30 mg to 1200 mg) every three weeks and the anti-PD-L1 antagonist antibody at a dose (e.g., a fixed dose) of between about 80 mg to about 2000 mg (e.g., between 80 mg to 2000 mg) every three weeks.

In another aspect, the invention provides uses of an anti-TIGIT antagonist antibody and atezolizumab in the manufacture of a medicament for use in a method of treating a subject or population of subjects having a cancer with a detectable expression level of PD-L1 (e.g., cervical cancer, e.g., Stage IVB, metastatic, recurrent, or persistent cervical cancer, e.g., a metastatic and/or recurrent PD-L1-positive cervical carcinoma), wherein the method comprises administering to the subject or population of subjects one or more dosing cycles of the medicament, wherein the medicament is formulated for administration of the anti-TIGIT antagonist antibody at a dose (e.g., a fixed dose) of 600 mg every three weeks and atezolizumab at a dose (e.g., a fixed dose) of 1200 mg every three weeks, and wherein the anti-TIGIT antagonist antibody comprises: a VH domain comprising the amino acid sequence of SEQ ID NO: 17 or 18 and a VL domain comprising the amino acid sequence of SEQ ID NO: 19, as described in further detail below. In some instances, the anti-TIGIT antagonist antibody has a VH domain having the amino acid sequence of SEQ ID NO: 17 and a VL domain having the amino acid sequence of SEQ ID NO: 19. In some instances, the anti-TIGIT antagonist antibody has a VH domain having the amino acid sequence of SEQ ID NO: 18 and a VL domain having the amino acid sequence of SEQ ID NO: 19.

In another aspect, the invention provides uses of an anti-TIGIT antagonist antibody and pembrolizumab in the manufacture of a medicament for use in a method of treating a subject or population of subjects having a cancer with a detectable expression level of PD-L1 (e.g., cervical cancer, e.g., Stage IVB, metastatic, recurrent, or persistent cervical cancer, e.g., a metastatic and/or recurrent PD-L1-positive cervical carcinoma), wherein the method comprises administering to the subject or population of subjects one or more dosing cycles of the medicament, wherein the medicament is formulated for administration of the anti-TIGIT antagonist antibody at a dose (e.g., a fixed dose) of 600 mg every three weeks and pembrolizumab at a dose (e.g., a fixed dose) of 200 mg every three weeks, and wherein the anti-TIGIT antagonist antibody comprises: a VH domain comprising the amino acid sequence of SEQ ID NO: 17 or 18 and a VL domain comprising the amino acid sequence of SEQ ID NO: 19, as described in further detail below. In some instances, the anti-TIGIT antagonist antibody has a VH domain having the amino acid sequence of SEQ ID NO: 17 and a VL domain having the amino acid sequence of SEQ ID NO: 19. In some instances, the anti-TIGIT antagonist antibody has a VH domain having the amino acid sequence of SEQ ID NO: 18 and a VL domain having the amino acid sequence of SEQ ID NO: 19.

In another aspect, the invention provides uses of an anti-TIGIT antagonist antibody and nivolumab in the manufacture of a medicament for use in a method of treating a subject or population of subjects having a cancer with a detectable expression level of PD-L1 (e.g., cervical cancer, e.g., Stage IVB, metastatic, recurrent, or persistent cervical cancer, e.g., a metastatic and/or recurrent PD-L1-positive cervical carcinoma), wherein the method comprises administering to the subject or population of subjects one or more dosing cycles of the medicament, wherein the medicament is formulated for administration of the anti-TIGIT antagonist antibody at a dose (e.g., a fixed dose) of 600 mg every three weeks and nivolumab at a dose (e.g., a fixed dose) of 240 mg every two weeks, and wherein the anti-TIGIT antagonist antibody comprises: a VH domain comprising the amino acid sequence of SEQ ID NO: 17 or 18 and a VL domain comprising the amino acid sequence of SEQ ID NO: 19, as described in further detail below. In some instances, the anti-TIGIT antagonist antibody has a VH domain having the amino acid sequence of SEQ ID NO: 17 and a VL domain having the amino acid sequence of SEQ ID NO: 19. In some instances, the anti-TIGIT antagonist antibody has a VH domain having the amino acid sequence of SEQ ID NO: 18 and a VL domain having the amino acid sequence of SEQ ID NO: 19.

In another aspect, the invention provides uses of an anti-TIGIT antagonist antibody and nivolumab in the manufacture of a medicament for use in a method of treating a subject or population of subjects having a cancer with a detectable expression level of PD-L1 (e.g., cervical cancer, e.g., Stage IVB, metastatic, recurrent, or persistent cervical cancer, e.g., a metastatic and/or recurrent PD-L1-positive cervical carcinoma), wherein the method comprises administering to the subject or population of subjects one or more dosing cycles of the medicament, wherein the medicament is formulated for administration of the anti-TIGIT antagonist antibody at a dose (e.g., a fixed dose) of 600 mg every three weeks and nivolumab at a dose (e.g., a fixed dose) of 480 mg every four weeks, and wherein the anti-TIGIT antagonist antibody comprises: a VH domain comprising the amino acid sequence of SEQ ID NO: 17 or 18 and a VL domain comprising the amino acid sequence of SEQ ID NO: 19, as described in further detail below. In some instances, the anti-TIGIT antagonist antibody has a VH domain having the amino acid sequence of SEQ ID NO: 17 and a VL domain having the amino acid sequence of SEQ ID NO: 19. In some instances, the anti-TIGIT antagonist antibody has a VH domain having the amino acid sequence of SEQ ID NO: 18 and a VL domain having the amino acid sequence of SEQ ID NO: 19.

In another aspect, the invention provides uses of an anti-TIGIT antagonist antibody in the manufacture of a medicament for use in a method of treating a subject or population of subjects having a cancer with a detectable expression level of PD-L1 (e.g., cervical cancer, e.g., Stage IVB, metastatic, recurrent, or persistent cervical cancer, e.g., a metastatic and/or recurrent PD-L1-positive cervical carcinoma), wherein the method comprises administering to the subject or population of subjects one or more dosing cycles of the medicament and atezolizumab, wherein the medicament is formulated for administration of the anti-TIGIT antagonist antibody at a dose (e.g., a fixed dose) of 600 mg every three weeks and atezolizumab is to be administered at a dose (e.g., a fixed dose) of 1200 mg every three weeks, and wherein the anti-TIGIT antagonist antibody comprises: a VH domain comprising the amino acid sequence of SEQ ID NO: 17 or 18 and a VL domain comprising the amino acid sequence of SEQ ID NO: 19, as described in further detail below. In some instances, the anti-TIGIT antagonist antibody has a VH domain having the amino acid sequence of SEQ ID NO: 17 and a VL domain having the amino acid sequence of SEQ ID NO: 19. In some instances, the anti-TIGIT antagonist antibody has a VH domain having the amino acid sequence of SEQ ID NO: 18 and a VL domain having the amino acid sequence of SEQ ID NO: 19.

In another aspect, the invention provides uses of an anti-TIGIT antagonist antibody in the manufacture of a medicament for use in a method of treating a subject or population of subjects having a cancer with a detectable expression level of PD-L1 (e.g., cervical cancer, e.g., Stage IVB, metastatic, recurrent, or persistent cervical cancer, e.g., a metastatic and/or recurrent PD-L1-positive cervical carcinoma), wherein the method comprises administering to the subject or population of subjects one or more dosing cycles of the medicament and pembrolizumab, wherein the medicament is formulated for administration of the anti-TIGIT antagonist antibody at a dose (e.g., a fixed dose) of 600 mg every three weeks and pembrolizumab is to be administered at a dose (e.g., a fixed dose) of 200 mg every three weeks, and wherein the anti-TIGIT antagonist antibody comprises: a VH domain comprising the amino acid sequence of SEQ ID NO: 17 or 18 and a VL domain comprising the amino acid sequence of SEQ ID NO: 19, as described in further detail below. In some instances, the anti-TIGIT antagonist antibody has a VH domain having the amino acid sequence of SEQ ID NO: 17 and a VL domain having the amino acid sequence of SEQ ID NO: 19. In some instances, the anti-TIGIT antagonist antibody has a VH domain having the amino acid sequence of SEQ ID NO: 18 and a VL domain having the amino acid sequence of SEQ ID NO: 19.

In another aspect, the invention provides uses of an anti-TIGIT antagonist antibody in the manufacture of a medicament for use in a method of treating a subject or population of subjects having a cancer with a detectable expression level of PD-L1 (e.g., cervical cancer, e.g., Stage IVB, metastatic, recurrent, or persistent cervical cancer, e.g., a metastatic and/or recurrent PD-L1-positive cervical carcinoma), wherein the method comprises administering to the subject or population of subjects one or more dosing cycles of the medicament and nivolumab, wherein the medicament is formulated for administration of the anti-TIGIT antagonist antibody at a dose (e.g., a fixed dose) of 600 mg every three weeks and nivolumab is to be administered at a dose (e.g., a fixed dose) of 240 mg every two weeks, and wherein the anti-TIGIT antagonist antibody comprises: a VH domain comprising the amino acid sequence of SEQ ID NO: 17 or 18 and a VL domain comprising the amino acid sequence of SEQ ID NO: 19, as described in further detail below. In some instances, the anti-TIGIT antagonist antibody has a VH domain having the amino acid sequence of SEQ ID NO: 17 and a VL domain having the amino acid sequence of SEQ ID NO: 19. In some instances, the anti-TIGIT antagonist antibody has a VH domain having the amino acid sequence of SEQ ID NO: 18 and a VL domain having the amino acid sequence of SEQ ID NO: 19. In another aspect, the invention provides uses of an anti-TIGIT antagonist antibody in the manufacture of a medicament for use in a method of treating a subject or population of subjects having a cancer with a detectable expression level of PD-L1 (e.g., cervical cancer, e.g., Stage IVB, metastatic, recurrent, or persistent cervical cancer, e.g., a metastatic and/or recurrent PD-L1-positive cervical carcinoma), wherein the method comprises administering to the subject or population of subjects one or more dosing cycles of the medicament and nivolumab, wherein the medicament is formulated for administration of the anti-TIGIT antagonist antibody at a dose (e.g., a fixed dose) of 600 mg every three weeks and nivolumab is to be administered at a dose (e.g., a fixed dose) of 480 mg every four weeks, and wherein the anti-TIGIT antagonist antibody comprises: a VH domain comprising the amino acid sequence of SEQ ID NO: 17 or 18 and a VL domain comprising the amino acid sequence of SEQ ID NO: 19, as described in further detail below. In some instances, the anti-TIGIT antagonist antibody has a VH domain having the amino acid sequence of SEQ ID NO: 17 and a VL domain having the amino acid sequence of SEQ ID NO: 19. In some instances, the anti-TIGIT antagonist antibody has a VH domain having the amino acid sequence of SEQ ID NO: 18 and a VL domain having the amino acid sequence of SEQ ID NO: 19.

In another aspect, the invention provides uses of an anti-TIGIT antagonist antibody in the manufacture of a medicament for use in a method of treating a subject or population of subjects having a cancer with a detectable expression level of PD-L1 (e.g., cervical cancer, e.g., Stage IVB, metastatic, recurrent, or persistent cervical cancer, e.g., a metastatic and/or recurrent PD-L1-positive cervical carcinoma), wherein the method comprises administering to the subject or population of subjects one or more dosing cycles of the medicament and atezolizumab, wherein the medicament is formulated for administration of the anti-TIGIT antagonist antibody at a dose (e.g., a fixed dose) of 600 mg every three weeks and atezolizumab is to be administered at a dose (e.g., a fixed dose) of 840 mg every two weeks, and wherein the anti-TIGIT antagonist antibody comprises: a VH domain comprising the amino acid sequence of SEQ ID NO: 17 or 18 and a VL domain comprising the amino acid sequence of SEQ ID NO: 19, as described in further detail below. In some instances, the anti-TIGIT antagonist antibody has a VH domain having the amino acid sequence of SEQ ID NO: 17 and a VL domain having the amino acid sequence of SEQ ID NO: 19. In some instances, the anti-TIGIT antagonist antibody has a VH domain having the amino acid sequence of SEQ ID NO: 18 and a VL domain having the amino acid sequence of SEQ ID NO: 19.

In another aspect, the invention provides uses of an anti-TIGIT antagonist antibody in the manufacture of a medicament for use in a method of treating a subject or population of subjects having a cancer with a detectable expression level of PD-L1 (e.g., cervical cancer, e.g., Stage IVB, metastatic, recurrent, or persistent cervical cancer, e.g., a metastatic and/or recurrent PD-L1-positive cervical carcinoma), wherein the method comprises administering to the subject or population of subjects one or more dosing cycles of the medicament and atezolizumab, wherein the medicament is formulated for administration of the anti-TIGIT antagonist antibody at a dose (e.g., a fixed dose) of 600 mg every three weeks and atezolizumab is to be administered at a dose (e.g., a fixed dose) of 1680 mg every four weeks, and wherein the anti-TIGIT antagonist antibody comprises: a VH domain comprising the amino acid sequence of SEQ ID NO: 17 or 18 and a VL domain comprising the amino acid sequence of SEQ ID NO: 19, as described in further detail below. In some instances, the anti-TIGIT antagonist antibody has a VH domain having the amino acid sequence of SEQ ID NO: 17 and a VL domain having the amino acid sequence of SEQ ID NO: 19. In some instances, the anti-TIGIT antagonist antibody has a VH domain having the amino acid sequence of SEQ ID NO: 18 and a VL domain having the amino acid sequence of SEQ ID NO: 19.

In another aspect, the invention provides uses of atezolizumab in the manufacture of a medicament for use in a method of treating a subject or population of subjects having a cancer with a detectable expression level of PD-L1 (e.g., cervical cancer, e.g., Stage IVB, metastatic, recurrent, or persistent cervical cancer, e.g., a metastatic and/or recurrent PD-L1-positive cervical carcinoma), wherein the method comprises administering to the subject or population of subjects one or more dosing cycles of the medicament and an anti-TIGIT antagonist antibody, wherein the medicament is formulated for administration of atezolizumab at a dose (e.g., a fixed dose) of 1200 mg every three weeks and the anti-TIGIT antagonist antibody is to be administered at a dose (e.g., a fixed dose) of 600 mg every three weeks, and wherein the anti-TIGIT antagonist antibody comprises: a VH domain comprising the amino acid sequence of SEQ ID NO: 17 or 18 and a VL domain comprising the amino acid sequence of SEQ ID NO: 19, as described in further detail below. In some instances, the anti-TIGIT antagonist antibody has a VH domain having the amino acid sequence of SEQ ID NO: 17 and a VL domain having the amino acid sequence of SEQ ID NO: 19. In some instances, the anti-TIGIT antagonist antibody has a VH domain having the amino acid sequence of SEQ ID NO: 18 and a VL domain having the amino acid sequence of SEQ ID NO: 19.

In another aspect, the invention provides uses of atezolizumab in the manufacture of a medicament for use in a method of treating a subject or population of subjects having a cancer with a detectable expression level of PD-L1 (e.g., cervical cancer, e.g., Stage IVB, metastatic, recurrent, or persistent cervical cancer, e.g., a metastatic and/or recurrent PD-L1-positive cervical carcinoma), wherein the method comprises administering to the subject or population of subjects one or more dosing cycles of the medicament and an anti-TIGIT antagonist antibody, wherein the medicament is formulated for administration of atezolizumab at a dose (e.g., a fixed dose) of 840 mg every two weeks and the anti-TIGIT antagonist antibody is to be administered at a dose (e.g., a fixed dose) of 600 mg every three weeks, and wherein the anti-TIGIT antagonist antibody comprises: a VH domain comprising the amino acid sequence of SEQ ID NO: 17 or 18 and a VL domain comprising the amino acid sequence of SEQ ID NO: 19, as described in further detail below. In some instances, the anti-TIGIT antagonist antibody has a VH domain having the amino acid sequence of SEQ ID NO: 17 and a VL domain having the amino acid sequence of SEQ ID NO: 19. In some instances, the anti-TIGIT antagonist antibody has a VH domain having the amino acid sequence of SEQ ID NO: 18 and a VL domain having the amino acid sequence of SEQ ID NO: 19.

In another aspect, the invention provides uses of pembrolizumab in the manufacture of a medicament for use in a method of treating a subject or population of subjects having a cancer with a detectable expression level of PD-L1 (e.g., cervical cancer, e.g., Stage IVB, metastatic, recurrent, or persistent cervical cancer, e.g., a metastatic and/or recurrent PD-L1-positive cervical carcinoma), wherein the method comprises administering to the subject or population of subjects one or more dosing cycles of the medicament and an anti-TIGIT antagonist antibody, wherein the medicament is formulated for administration of pembrolizumab at a dose (e.g., a fixed dose) of 200 mg every three weeks and the anti-TIGIT antagonist antibody is to be administered at a dose (e.g., a fixed dose) of 600 mg every three weeks, and wherein the anti-TIGIT antagonist antibody comprises: a VH domain comprising the amino acid sequence of SEQ ID NO: 17 or 18 and a VL domain comprising the amino acid sequence of SEQ ID NO: 19, as described in further detail below. In some instances, the anti-TIGIT antagonist antibody has a VH domain having the amino acid sequence of SEQ ID NO: 17 and a VL domain having the amino acid sequence of SEQ ID NO: 19. In some instances, the anti-TIGIT antagonist antibody has a VH domain having the amino acid sequence of SEQ ID NO: 18 and a VL domain having the amino acid sequence of SEQ ID NO: 19.

In another aspect, the invention provides uses of nivolumab in the manufacture of a medicament for use in a method of treating a subject or population of subjects having a cancer with a detectable expression level of PD-L1 (e.g., cervical cancer, e.g., Stage IVB, metastatic, recurrent, or persistent cervical cancer, e.g., a metastatic and/or recurrent PD-L1-positive cervical carcinoma), wherein the method comprises administering to the subject or population of subjects one or more dosing cycles of the medicament and an anti-TIGIT antagonist antibody, wherein the medicament is formulated for administration of nivolumab at a dose (e.g., a fixed dose) of 240 mg every two weeks and the anti-TIGIT antagonist antibody is to be administered at a dose (e.g., a fixed dose) of 600 mg every three weeks, and wherein the anti-TIGIT antagonist antibody comprises: a VH domain comprising the amino acid sequence of SEQ ID NO: 17 or 18 and a VL domain comprising the amino acid sequence of SEQ ID NO: 19, as described in further detail below. In some instances, the anti-TIGIT antagonist antibody has a VH domain having the amino acid sequence of SEQ ID NO: 17 and a VL domain having the amino acid sequence of SEQ ID NO: 19. In some instances, the anti-TIGIT antagonist antibody has a VH domain having the amino acid sequence of SEQ ID NO: 18 and a VL domain having the amino acid sequence of SEQ ID NO: 19.

In another aspect, the invention provides uses of nivolumab in the manufacture of a medicament for use in a method of treating a subject or population of subjects having a cancer with a detectable expression level of PD-L1 (e.g., cervical cancer, e.g., Stage IVB, metastatic, recurrent, or persistent cervical cancer, e.g., a metastatic and/or recurrent PD-L1-positive cervical carcinoma), wherein the method comprises administering to the subject or population of subjects one or more dosing cycles of the medicament and an anti-TIGIT antagonist antibody, wherein the medicament is formulated for administration of nivolumab at a dose (e.g., a fixed dose) of 480 mg every four weeks and the anti-TIGIT antagonist antibody is to be administered at a dose (e.g., a fixed dose) of 600 mg every three weeks, and wherein the anti-TIGIT antagonist antibody comprises: a VH domain comprising the amino acid sequence of SEQ ID NO: 17 or 18 and a VL domain comprising the amino acid sequence of SEQ ID NO: 19, as described in further detail below. In some instances, the anti-TIGIT antagonist antibody has a VH domain having the amino acid sequence of SEQ ID NO: 17 and a VL domain having the amino acid sequence of SEQ ID NO: 19. In some instances, the anti-TIGIT antagonist antibody has a VH domain having the amino acid sequence of SEQ ID NO: 18 and a VL domain having the amino acid sequence of SEQ ID NO: 19.

In another aspect, the invention provides uses of atezolizumab in the manufacture of a medicament for use in a method of treating a subject or population of subjects having a cancer with a detectable expression level of PD-L1 (e.g., cervical cancer, e.g., Stage IVB, metastatic, recurrent, or persistent cervical cancer, e.g., a metastatic and/or recurrent PD-L1-positive cervical carcinoma), wherein the method comprises administering to the subject or population of subjects one or more dosing cycles of the medicament and an anti-TIGIT antagonist antibody, wherein the medicament is formulated for administration of atezolizumab at a dose (e.g., a fixed dose) of 1680 mg every four weeks and the anti-TIGIT antagonist antibody is to be administered at a dose (e.g., a fixed dose) of 600 mg every three weeks, and wherein the anti-TIGIT antagonist antibody comprises: a VH domain comprising the amino acid sequence of SEQ ID NO: 17 or 18 and a VL domain comprising the amino acid sequence of SEQ ID NO: 19, as described in further detail below. In some instances, the anti-TIGIT antagonist antibody has a VH domain having the amino acid sequence of SEQ ID NO: 17 and a VL domain having the amino acid sequence of SEQ ID NO: 19. In some instances, the anti-TIGIT antagonist antibody has a VH domain having the amino acid sequence of SEQ ID NO: 18 and a VL domain having the amino acid sequence of SEQ ID NO: 19.

In another aspect, the invention provides uses of tiragolumab and atezolizumab in the manufacture of a medicament for use in a method of treating a subject or population of subjects having a cancer with a detectable expression level of PD-L1 (e.g., cervical cancer, e.g., Stage IVB, metastatic, recurrent, or persistent cervical cancer, e.g., a metastatic and/or recurrent PD-L1-positive cervical carcinoma), wherein the method comprises administering to the subject or population of subjects one or more dosing cycles of the medicament, wherein the medicament is formulated for administration of tiragolumab at a dose (e.g., a fixed dose) of 600 mg every three weeks and atezolizumab at a dose (e.g., a fixed dose) of 1200 mg every three weeks.

In another aspect, the invention provides uses of tiragolumab and atezolizumab in the manufacture of a medicament for use in a method of treating a subject or population of subjects having a cancer with a detectable expression level of PD-L1 (e.g., cervical cancer, e.g., Stage IVB, metastatic, recurrent, or persistent cervical cancer, e.g., a metastatic and/or recurrent PD-L1-positive cervical carcinoma), wherein the method comprises administering to the subject or population of subjects one or more dosing cycles of the medicament, wherein the medicament is formulated for administration of tiragolumab at a dose (e.g., a fixed dose) of 600 mg every three weeks and atezolizumab at a dose (e.g., a fixed dose) of 840 mg every two weeks.

In another aspect, the invention provides uses of tiragolumab and atezolizumab in the manufacture of a medicament for use in a method of treating a subject or population of subjects having a cancer with a detectable expression level of PD-L1 (e.g., cervical cancer, e.g., Stage IVB, metastatic, recurrent, or persistent cervical cancer, e.g., a metastatic and/or recurrent PD-L1-positive cervical carcinoma), wherein the method comprises administering to the subject or population of subjects one or more dosing cycles of the medicament, wherein the medicament is formulated for administration of tiragolumab at a dose (e.g., a fixed dose) of 600 mg every three weeks and atezolizumab at a dose (e.g., a fixed dose) of 840 mg every two weeks.

In another aspect, the invention provides uses of tiragolumab and pembrolizumab in the manufacture of a medicament for use in a method of treating a subject or population of subjects having a cancer with a detectable expression level of PD-L1 (e.g., cervical cancer, e.g., Stage IVB, metastatic, recurrent, or persistent cervical cancer, e.g., a metastatic and/or recurrent PD-L1-positive cervical carcinoma), wherein the method comprises administering to the subject or population of subjects one or more dosing cycles of the medicament, wherein the medicament is formulated for administration of tiragolumab at a dose (e.g., a fixed dose) of 600 mg every three weeks and pembrolizumab at a dose (e.g., a fixed dose) of 200 mg every three weeks.

In another aspect, the invention provides uses of tiragolumab and nivolumab in the manufacture of a medicament for use in a method of treating a subject or population of subjects having a cancer with a detectable expression level of PD-L1 (e.g., cervical cancer, e.g., Stage IVB, metastatic, recurrent, or persistent cervical cancer, e.g., a metastatic and/or recurrent PD-L1-positive cervical carcinoma), wherein the method comprises administering to the subject or population of subjects one or more dosing cycles of the medicament, wherein the medicament is formulated for administration of tiragolumab at a dose (e.g., a fixed dose) of 600 mg every three weeks and nivolumab at a dose (e.g., a fixed dose) of 240 mg every two weeks.

In another aspect, the invention provides uses of tiragolumab and nivolumab in the manufacture of a medicament for use in a method of treating a subject or population of subjects having a cancer with a detectable expression level of PD-L1 (e.g., cervical cancer, e.g., Stage IVB, metastatic, recurrent, or persistent cervical cancer, e.g., a metastatic and/or recurrent PD-L1-positive cervical carcinoma), wherein the method comprises administering to the subject or population of subjects one or more dosing cycles of the medicament, wherein the medicament is formulated for administration of tiragolumab at a dose (e.g., a fixed dose) of 600 mg every three weeks and nivolumab at a dose (e.g., a fixed dose) of 480 mg every four weeks.

In another aspect, the invention provides uses of tiragolumab in the manufacture of a medicament for use in a method of treating a subject or population of subjects having a cancer with a detectable expression level of PD-L1 (e.g., cervical cancer, e.g., Stage IVB, metastatic, recurrent, or persistent cervical cancer, e.g., a metastatic and/or recurrent PD-L1-positive cervical carcinoma), wherein the method comprises administering to the subject or population of subjects one or more dosing cycles of the medicament and atezolizumab, wherein the medicament is formulated for administration of tiragolumab at a dose (e.g., a fixed dose) of 600 mg every three weeks and atezolizumab is to be administered at a dose (e.g., a fixed dose) of 1200 mg every three weeks.

In another aspect, the invention provides uses of tiragolumab in the manufacture of a medicament for use in a method of treating a subject or population of subjects having a cancer with a detectable expression level of PD-L1 (e.g., cervical cancer, e.g., Stage IVB, metastatic, recurrent, or persistent cervical cancer, e.g., a metastatic and/or recurrent PD-L1-positive cervical carcinoma), wherein the method comprises administering to the subject or population of subjects one or more dosing cycles of the medicament and atezolizumab, wherein the medicament is formulated for administration of tiragolumab at a dose (e.g., a fixed dose) of 600 mg every three weeks and atezolizumab is to be administered at a dose (e.g., a fixed dose) of 840 mg every two weeks.

In another aspect, the invention provides uses of tiragolumab in the manufacture of a medicament for use in a method of treating a subject or population of subjects having a cancer with a detectable expression level of PD-L1 (e.g., cervical cancer, e.g., Stage IVB, metastatic, recurrent, or persistent cervical cancer, e.g., a metastatic and/or recurrent PD-L1-positive cervical carcinoma), wherein the method comprises administering to the subject or population of subjects one or more dosing cycles of the medicament and atezolizumab, wherein the medicament is formulated for administration of tiragolumab at a dose (e.g., a fixed dose) of 600 mg every three weeks and atezolizumab is to be administered at a dose (e.g., a fixed dose) of 840 mg every two weeks.

In another aspect, the invention provides uses of tiragolumab in the manufacture of a medicament for use in a method of treating a subject or population of subjects having a cancer with a detectable expression level of PD-L1 (e.g., cervical cancer, e.g., Stage IVB, metastatic, recurrent, or persistent cervical cancer, e.g., a metastatic and/or recurrent PD-L1-positive cervical carcinoma), wherein the method comprises administering to the subject or population of subjects one or more dosing cycles of the medicament and pembrolizumab, wherein the medicament is formulated for administration of tiragolumab at a dose (e.g., a fixed dose) of 600 mg every three weeks and pembrolizumab is to be administered at a dose (e.g., a fixed dose) of 200 mg every three weeks.

In another aspect, the invention provides uses of tiragolumab in the manufacture of a medicament for use in a method of treating a subject or population of subjects having a cancer with a detectable expression level of PD-L1 (e.g., cervical cancer, e.g., Stage IVB, metastatic, recurrent, or persistent cervical cancer, e.g., a metastatic and/or recurrent PD-L1-positive cervical carcinoma), wherein the method comprises administering to the subject or population of subjects one or more dosing cycles of the medicament and pembrolizumab, wherein the medicament is formulated for administration of tiragolumab at a dose (e.g., a fixed dose) of 600 mg every three weeks and nivolumab is to be administered at a dose (e.g., a fixed dose) of 240 mg every two weeks.

In another aspect, the invention provides uses of tiragolumab in the manufacture of a medicament for use in a method of treating a subject or population of subjects having a cancer with a detectable expression level of PD-L1 (e.g., cervical cancer, e.g., Stage IVB, metastatic, recurrent, or persistent cervical cancer, e.g., a metastatic and/or recurrent PD-L1-positive cervical carcinoma), wherein the method comprises administering to the subject or population of subjects one or more dosing cycles of the medicament and pembrolizumab, wherein the medicament is formulated for administration of tiragolumab at a dose (e.g., a fixed dose) of 600 mg every three weeks and nivolumab is to be administered at a dose (e.g., a fixed dose) of 480 mg every four weeks.

In another aspect, the invention provides uses of atezolizumab in the manufacture of a medicament for use in a method of treating a subject or population of subjects having a cancer with a detectable expression level of PD-L1 (e.g., cervical cancer, e.g., Stage IVB, metastatic, recurrent, or persistent cervical cancer, e.g., a metastatic and/or recurrent PD-L1-positive cervical carcinoma), wherein the method comprises administering to the subject or population of subjects one or more dosing cycles of the medicament and tiragolumab, wherein the medicament is formulated for administration of atezolizumab at a dose (e.g., a fixed dose) of 1200 mg every three weeks and tiragolumab is to be administered at a dose (e.g., a fixed dose) of 600 mg every three weeks.

In another aspect, the invention provides uses of atezolizumab in the manufacture of a medicament for use in a method of treating a subject or population of subjects having a cancer with a detectable expression level of PD-L1 (e.g., cervical cancer, e.g., Stage IVB, metastatic, recurrent, or persistent cervical cancer, e.g., a metastatic and/or recurrent PD-L1-positive cervical carcinoma), wherein the method comprises administering to the subject or population of subjects one or more dosing cycles of the medicament and tiragolumab, wherein the medicament is formulated for administration of atezolizumab at a dose (e.g., a fixed dose) of 1200 mg every three weeks and tiragolumab is to be administered at a dose (e.g., a fixed dose) of 840 mg every two weeks.

In another aspect, the invention provides uses of atezolizumab in the manufacture of a medicament for use in a method of treating a subject or population of subjects having a cancer with a detectable expression level of PD-L1 (e.g., cervical cancer, e.g., Stage IVB, metastatic, recurrent, or persistent cervical cancer, e.g., a metastatic and/or recurrent PD-L1-positive cervical carcinoma), wherein the method comprises administering to the subject or population of subjects one or more dosing cycles of the medicament and tiragolumab, wherein the medicament is formulated for administration of atezolizumab at a dose (e.g., a fixed dose) of 1200 mg every three weeks and tiragolumab is to be administered at a dose (e.g., a fixed dose) of 1680 mg every four weeks.

In any of the methods, uses, or compositions for use described herein, the anti-TIGIT antagonist antibody (e.g., an anti-TIGIT antagonist antibody as disclosed herein, e.g., tiragolumab) and PD-1 axis binding antagonist (e.g., anti-PD-L1 antagonist antibody (e.g., atezolizumab) or anti-PD-1 antagonist antibody (e.g., MDX-1106 (nivolumab) or MK-3475 (pembrolizumab, previously known as lambrolizumab))), or a medicament thereof, may be administered in conjunction with (either separately or together), one or more additional anti-cancer therapeutic agent(s) (e.g., a chemotherapeutic agent, a cytotoxic agent, a growth inhibitory agent, a radiotherapy/radiation therapy, and/or an anti-hormonal agent, such as those recited herein above).

In any of the methods, uses, or compositions for use described herein, the anti-TIGIT antagonist antibody (e.g., an anti-TIGIT antagonist antibody as disclosed herein, e.g., tiragolumab) and PD-1 axis binding antagonist (e.g., anti-PD-L1 antagonist antibody (e.g., atezolizumab) or anti-PD-1 antagonist antibody (e.g., MDX-1106 (nivolumab) or MK-3475 (pembrolizumab, previously known as lambrolizumab))), or a medicament thereof, is for treating a subject or population of subjects having a cervical cancer. In some instances, the cervical cancer is Stage IVB, metastatic, recurrent, or persistent cervical cancer. In some instances, the cervical cancer is a metastatic and/or recurrent PD-L1-positive cervical carcinoma. The cancer may be at an early or late stage.

In some instances, in any of the methods, uses, or compositions for use described herein, the detectable expression level of PD-L1 is a detectable protein expression level of PD-L1. In some instances, the detectable protein expression level of PD-L1 has been determined by an immunohistochemical (IHC) assay. In some instances, the protein expression level of PD-L1 is detected using an anti-PD-L1 antibody suitable for staining. In some instances, the tumor sample is a formalin-fixed, paraffin-embedded (FFPE) tumor sample.

In some instances, in any of the methods, uses, or compositions for use described herein, the detectable expression level of PD-L1 is a detectable nucleic acid expression level of PD-L1. In some instances, the detectable nucleic acid expression level of PD-L1 has been determined by RNA-seq, RT-qPCR, qPCR, multiplex qPCR or RT-qPCR, microarray analysis, SAGE, MassARRAY technique, ISH, or a combination thereof.

In some instances, in any of the methods, uses, or compositions for use described herein, the cervical cancer is a squamous cell carcinoma, adenosquamous carcinoma, or adenocarcinoma. In some instances, the cervical cancer is Stage IVB, metastatic, recurrent, or persistent. In some instances, the cervical cancer is a metastatic and/or recurrent PD-L1-positive cervical carcinoma. In some instances, the subject or population of subjects has not received prior therapy. In some instances, the subject or population of subjects has received at least one line of prior therapy. In some instances, the subject or population of subjects has received two lines of prior therapy. In some instances, the subject or population of subjects has received at least one but no more than two prior systemic therapies and/or for whom no acceptable standard of care exists. In some instances, the subject or population of subjects has not received more than two lines of prior therapy. In some instances, the prior therapy is chemotherapy, surgery, and/or radiotherapy. In some instances, the prior therapy is an immunotherapy.

In some instances, in any of the methods, uses, or compositions for use described herein, administration of the anti-TIGIT antagonist antibody and PD-1 axis binding antagonist (e.g., anti-PD-L1 antagonist antibody (e.g., atezolizumab) or anti-PD-1 antagonist antibody (e.g., MDX-1106 (nivolumab) or MK-3475 (pembrolizumab, previously known as lambrolizumab))) results in a clinical response. In some instances, the clinical response is an increase in the objective response rate (ORR) of the subject or population of subjects as compared to a reference ORR. In some instances, the reference ORR is at least about 14.6% to about 26%. In some instances, the reference ORR is the median ORR of a population of subjects who have received a treatment comprising a PD-1 axis binding antagonist (e.g., an anti-PD-L1 antagonist antibody (e.g., atezolizumab) or an anti-PD-1 antagonist antibody (e.g., MDX-1106 (nivolumab) or MK-3475 (pembrolizumab, previously known as lambrolizumab))) without an anti-TIGIT antagonist antibody. In some instances, the clinical response is an increase in the PFS of the subject or population of subjects as compared to a reference PFS time. In some instances, wherein the reference PFS time is the median PFS time of a population of subjects who have received a treatment comprising a PD-1 axis binding antagonist (e.g., an anti-PD-L1 antagonist antibody (e.g., atezolizumab) or an anti-PD-1 antagonist antibody (e.g., MDX-1106 (nivolumab) or MK-3475 (pembrolizumab, previously known as lambrolizumab))) without an anti-TIGIT antagonist antibody. In some instances, the clinical response is an increase in the duration of response (DOR) of the subject or population of subjects compared to a reference DOR time. In some instances, wherein the reference DOR time is the median DOR time of a population of subjects who have received a treatment comprising a PD-1 axis binding antagonist (e.g., an anti-PD-L1 antagonist antibody (e.g., atezolizumab) or an anti-PD-1 antagonist antibody (e.g., MDX-1106 (nivolumab) or MK-3475 (pembrolizumab, previously known as lambrolizumab))) without an anti-TIGIT antagonist antibody. In some instances, the clinical response is an increase in the OS.

Diagnostic Methods and Uses Relating to Cervical Cancer

The invention provides methods for selecting a therapy for a subject or population of subjects having a cancer (e.g., cervical cancer, e.g., Stage IVB, metastatic, recurrent, or persistent cervical cancer, e.g., a metastatic and/or recurrent PD-L1-positive cervical carcinoma), wherein therapy is guided by diagnostic methods that involve determining the presence and/or expression levels/amount of one or more biomarkers (e.g., PD-L1, TIGIT, activated T cells, or cytokines) in a sample (e.g., a tumor sample or a blood sample) obtained from the subject or population of subjects.

Additionally provided herein are methods for identifying a subject or population of subjects having a cancer (e.g., cervical cancer, e.g., Stage IVB, metastatic, recurrent, or persistent cervical cancer, e.g., a metastatic and/or recurrent PD-L1-positive cervical carcinoma) who may benefit from a treatment comprising an anti-TIGIT antagonist antibody and a PD-1 axis binding antagonist (e.g., an anti-PD-L1 antagonist antibody (e.g., atezolizumab) or an anti-PD-1 antagonist antibody (e.g., MDX-1106 (nivolumab) or MK-3475 (pembrolizumab, previously known as lambrolizumab))), wherein identification is guided by diagnostic methods that involve determining the presence and/or expression levels/amount of one or more biomarkers (e.g., PD-L1, TIGIT, activated T cells, or cytokines) in a sample (e.g., a tumor sample or a blood sample) obtained from the subject or population of subjects.

Additionally provided herein are methods for assessing responsiveness to a therapy for a subject or population of subjects having a cancer (e.g., cervical cancer, e.g., Stage IVB, metastatic, recurrent, or persistent cervical cancer, e.g., a metastatic and/or recurrent PD-L1-positive cervical carcinoma), wherein further therapy is guided by diagnostic methods that involve determining the presence and/or expression levels/amount of one or more biomarkers (e.g., PD-L1, TIGIT, activated T cells, or cytokines) in a sample (e.g., a tumor sample or a blood sample) obtained from the subject or population of subjects.

Additionally provided herein are methods for optimizing a therapy for a subject or population of subjects having a cancer (e.g., cervical cancer, e.g., Stage IVB, metastatic, recurrent, or persistent cervical cancer, e.g., a metastatic and/or recurrent PD-L1-positive cervical carcinoma), wherein further therapy is guided by diagnostic methods that involve determining the presence and/or expression levels/amount of one or more biomarkers (e.g., PD-L1, TIGIT, activated T cells, or cytokines) in a sample (e.g., a tumor sample or a blood sample) obtained from the subject or population of subjects.

Biomarkers for use in the methods described herein can include, but are not limited to, PD-L1 and/or TIGIT expression on tissues (e.g., tumor tissues) or in blood (e.g., whole blood), germline and somatic mutations from tissue (e.g., tumor tissue) and/or from circulating tumor DNA in blood (including, but not limited to, mutation load, MSI, and MMR defects), identified through WGS and/or NGS, analysis of genes (e.g., CD274) or gene signatures associated with tumor immunobiology (e.g., TEFF), HPV alterations, lymphocyte subpopulations, T cell-receptor repertoire, cytokines associated with T-cell activation, and plasma derived cytokines. In some instances, the biomarker is PD-L1. In some instances, the sample is a tumor sample (e.g., a formalin-fixed, paraffin-embedded (FFPE) tumor sample).

In some instances, the method includes determining the presence and/or expression levels/amount of a biomarker (e.g., PD-L1, TIGIT, activated T cells, or cytokines) in a sample (e.g., a tumor sample or a blood sample) from the subject or population of subjects, and administering to the subject or population of subjects one or more dosing cycles of an anti-TIGIT antagonist antibody (e.g., an anti-TIGIT antagonist antibody disclosed herein, e.g., tiragolumab) at a dose (e.g., a fixed dose) of between about 30 mg to about 1200 mg (e.g., between 30 mg to 1200 mg) every three weeks and one or more dosing cycles of a PD-1 axis binding antagonist (e.g., an anti-PD-L1 antagonist antibody (e.g., atezolizumab)) at a dose (e.g., a fixed dose) of between about 80 mg to about 2000 mg (e.g., between 80 mg to 2000 mg) every three weeks. In some instances, the method includes determining the presence and/or expression levels/amount of a biomarker (e.g., PD-L1, TIGIT, activated T cells, or cytokines) in a sample (e.g., a tumor sample or a blood sample) from the subject or population of subjects, and administering to the subject or population of subjects one or more dosing cycles of an anti-TIGIT antagonist antibody (e.g., an anti-TIGIT antagonist antibody disclosed herein, e.g., tiragolumab) at a dose (e.g., a fixed dose) of about 600 mg (e.g., 600 mg) every three weeks and one or more dosing cycles of a PD-1 axis binding antagonist (e.g., an anti-PD-L1 antagonist antibody (e.g., atezolizumab)) at a dose (e.g., a fixed dose) of 840 mg every two weeks. In some instances, the method includes determining the presence and/or expression levels/amount of a biomarker (e.g., PD-L1, TIGIT, activated T cells, or cytokines) in a sample (e.g., a tumor sample or a blood sample) from the subject or population of subjects, and administering to the subject or population of subjects one or more dosing cycles of an anti-TIGIT antagonist antibody (e.g., an anti-TIGIT antagonist antibody disclosed herein, e.g., tiragolumab) at a dose (e.g., a fixed dose) of about 600 mg (e.g., 600 mg) every three weeks and one or more dosing cycles of a PD-1 axis binding antagonist (e.g., an anti-PD-L1 antagonist antibody (e.g., atezolizumab)) at a dose (e.g., a fixed dose) of 1200 mg every three weeks. In some instances, the method includes determining the presence and/or expression levels/amount of a biomarker (e.g., PD-L1, TIGIT, activated T cells, or cytokines) in a sample (e.g., a tumor sample or a blood sample) from the subject or population of subjects, and administering to the subject or population of subjects one or more dosing cycles of an anti-TIGIT antagonist antibody (e.g., an anti-TIGIT antagonist antibody disclosed herein, e.g., tiragolumab) at a dose (e.g., a fixed dose) of about 600 mg (e.g., 600 mg) every three weeks and one or more dosing cycles of a PD-1 axis binding antagonist (e.g., an anti-PD-L1 antagonist antibody (e.g., atezolizumab)) at a dose (e.g., a fixed dose) of 1680 mg every four weeks.

Presence and/or expression levels/amount of a biomarker (e.g., PD-L1, TIGIT, activated T cells, or cytokines) can be determined qualitatively and/or quantitatively based on any suitable criterion known in the art, including but not limited to proteins, protein fragments, DNA, mRNA, cDNA, and/or gene copy number.

In some instances, expression levels or amount of a biomarker is a detectable protein expression level of PD-L1 in a tumor sample (e.g., a FFPE tumor sample) from the subject or population of subjects. In some instances, the PD-L1 protein expression level has been determined by an immunohistochemical (IHC) assay. In some instances, the tumor sample is an FFPE tumor sample.

In some instances, the tumor sample (e.g., FFPE tumor sample) from the subject or population of subjects has been determined to have a detectable expression level of PD-L1. In some instances, the tumor sample (e.g., FFPE tumor sample) from the subject or population of subjects has been determined to have a detectable expression level of PD-L1 in tumor-infiltrating immune cells. In some instances, the tumor sample is an FFPE tumor sample.

In some instances, the expression levels or amount of a biomarker is a detectable nucleic acid expression level of PD-L1 in a tumor sample (e.g., FFPE tumor sample) from the subject or population of subjects. In some instances, the PD-L1 nucleic acid expression level has been determined by RNA-seq, RT-qPCR, qPCR, multiplex qPCR, or RT-qPCR, microarray analysis, serial analysis of gene expression (SAGE), MassARRAY® technique, in situ hybridization (ISH), or a combination thereof. In some instances, the tumor sample is an FFPE tumor sample.

In some instances, the presence and/or expression levels/amount of the biomarker (e.g., PD-L1, TIGIT, activated T cells, or cytokines) in a sample (e.g., a tumor sample or a blood sample) from a subject or population of subjects selects the subject or population of subjects as eligible for therapy with an anti-TIGIT antagonist antibody and a PD-1 axis binding antagonist (e.g., an anti-PD-L1 antagonist antibody (e.g., atezolizumab) or an anti-PD-1 antagonist antibody (e.g., MDX-1106 (nivolumab) or MK-3475 (pembrolizumab, previously known as lambrolizumab))), for example, where a detectable expression level of PD-L1 is a biomarker for selection of individuals. In some instances, the sample is selected from the group consisting of a tissue sample, a whole blood sample, a serum sample, and a plasma sample. In some instances, the tissue sample is a tumor sample (e.g., FFPE tumor sample). In some instances, the tumor sample comprises tumor-infiltrating immune cells, tumor cells, stromal cells, and any combinations thereof. In some instances, the tumor sample is an FFPE tumor sample.

In one aspect, the invention provides methods for selecting a therapy for a subject or population of subjects having a cancer (e.g., cervical cancer, e.g., Stage IVB, metastatic, recurrent, or persistent cervical cancer, e.g., a metastatic and/or recurrent PD-L1-positive cervical carcinoma) by obtaining a tumor sample (e.g., a biopsy) from the subject or population of subjects, detecting the protein expression level of PD-L1 in the tumor sample by an IHC assay using an anti-PD-L1 antibody suitable for staining, and identifying the subject or population of subjects as one who is likely to benefit from a therapy comprising one or more dosing cycles of an anti-TIGIT antagonist antibody administered at a dose (e.g., a fixed dose) of 600 mg every three weeks and atezolizumab administered at a dose (e.g., a fixed dose) of 1200 mg every three weeks based on PD-L1 expression in the tumor sample having been detected, wherein the anti-TIGIT antagonist antibody comprises: a VH domain comprising the amino acid sequence of SEQ ID NO: 17 or 18, and a VL domain comprising the amino acid sequence of SEQ ID NO: 19. In some instances, the anti-TIGIT antagonist antibody has a VH domain having the amino acid sequence of SEQ ID NO: 17 and a VL domain having the amino acid sequence of SEQ ID NO: 19. In some instances, the anti-TIGIT antagonist antibody has a VH domain having the amino acid sequence of SEQ ID NO: 18 and a VL domain having the amino acid sequence of SEQ ID NO: 19. In some instances, the therapy comprises one or more dosing cycles of an anti-TIGIT antagonist antibody administered at a dose of 600 mg every three weeks and atezolizumab administered at a dose of 1680 mg every four weeks. In some instances, the therapy comprises one or more dosing cycles of an anti-TIGIT antagonist antibody administered at a dose of 600 mg every three weeks and atezolizumab administered at a dose of 840 mg every two weeks. In some instances, the method further includes administering to the identified subject or population of subjects the therapy. In another aspect, the invention provides methods for selecting a therapy for a subject or population of subjects having a cancer (e.g., cervical cancer, e.g., Stage IVB, metastatic, recurrent, or persistent cervical cancer, e.g., a metastatic and/or recurrent PD-L1-positive cervical carcinoma) by obtaining a tumor sample (e.g., a biopsy) from the subject or population of subjects, detecting the protein expression level of PD-L1 in the tumor sample by an IHC assay using an anti-PD-L1 antibody suitable for staining, and identifying the subject or population of subjects as one who is likely to benefit from a therapy comprising one or more dosing cycles of tiragolumab administered at a dose (e.g., a fixed dose) of 600 mg every three weeks and atezolizumab administered at a dose (e.g., a fixed dose) of 1200 mg every three weeks based on PD-L1 expression in the tumor sample having been detected. In some instances, the therapy may further include, or be administered in conjunction with (either separately or together), one or more additional anti-cancer therapeutic agent(s) (e.g., an immunomodulatory agent (e.g., an agent that decreases or inhibits one or more immune co-inhibitory receptors (e.g., one or more immune co-inhibitory receptors selected from TIGIT, PD-L1, PD-1, CTLA-4, LAG3, TIM3, BTLA, and/or VISTA), such as a CTLA-4 antagonist, e.g., an anti-CTLA-4 antagonist antibody (e.g., ipilimumab (YERVOY®)), or an agent that increases or activates one or more immune co-stimulatory receptors (e.g., one or more immune co-stimulatory receptors selected from CD226, OX-40, CD28, CD27, CD137, HVEM, and/or GITR), such as an OX-40 agonist, e.g., an OX-40 agonist antibody), a chemotherapeutic agent, a cytotoxic agent, a growth inhibitory agent, a radiotherapy/radiation therapy, and/or an anti-hormonal agent, such as those recited herein above).

In another aspect, the invention provides methods for selecting a therapy for a subject or population of subjects having a cancer (e.g., cervical cancer, e.g., Stage IVB, metastatic, recurrent, or persistent cervical cancer, e.g., a metastatic and/or recurrent PD-L1-positive cervical carcinoma) by obtaining a tumor sample (e.g., a biopsy) from the subject or population of subjects, detecting the protein expression level of PD-L1 in the tumor sample by an IHC assay using an anti-PD-L1 antibody suitable for staining, and identifying the subject or population of subjects as one who is likely to benefit from a therapy comprising one or more dosing cycles of an anti-TIGIT antagonist antibody administered at a dose (e.g., a fixed dose) of 600 mg every three weeks and atezolizumab administered at a dose (e.g., a fixed dose) of 1200 mg every three weeks based on PD-L1 expression in the tumor sample having been detected, wherein the anti-TIGIT antagonist antibody comprises: a VH domain comprising the amino acid sequence of SEQ ID NO: 17 or 18, and a VL domain comprising the amino acid sequence of SEQ ID NO: 19. In some instances, the anti-TIGIT antagonist antibody has a VH domain having the amino acid sequence of SEQ ID NO: 17 and a VL domain having the amino acid sequence of SEQ ID NO: 19. In some instances, the anti-TIGIT antagonist antibody has a VH domain having the amino acid sequence of SEQ ID NO: 18 and a VL domain having the amino acid sequence of SEQ ID NO: 19. In some instances, the therapy comprises one or more dosing cycles of an anti-TIGIT antagonist antibody administered at a dose of 600 mg every three weeks and atezolizumab administered at a dose of 1680 mg every four weeks. In some instances, the therapy comprises one or more dosing cycles of an anti-TIGIT antagonist antibody administered at a dose of 600 mg every three weeks and atezolizumab administered at a dose of 840 mg every two weeks. In another aspect, the invention provides methods for selecting a therapy for a subject or population of subjects having a cancer (e.g., cervical cancer, e.g., Stage IVB, metastatic, recurrent, or persistent cervical cancer, e.g., a metastatic and/or recurrent PD-L1-positive cervical carcinoma) by obtaining a tumor sample (e.g., a biopsy) from the subject or population of subjects, detecting the protein expression level of PD-L1 in the tumor sample by an IHC assay using an anti-PD-L1 antibody suitable for staining, and identifying the subject or population of subjects as one who is likely to benefit from a therapy comprising one or more dosing cycles of tiragolumab administered at a dose (e.g., a fixed dose) of 600 mg every three weeks and atezolizumab administered at a dose (e.g., a fixed dose) of 1200 mg every three weeks based on PD-L1 expression in the tumor sample having been detected. In some instances, the method further includes administering to the identified subject or population of subjects the therapy. In some instances, the therapy may further include, or be administered in conjunction with (either separately or together), one or more additional anti-cancer therapeutic agent(s) (e.g., an immunomodulatory agent (e.g., an agent that decreases or inhibits one or more immune co-inhibitory receptors (e.g., one or more immune co-inhibitory receptors selected from TIGIT, PD-L1, PD-1, CTLA-4, LAG3, TIM3, BTLA, and/or VISTA), such as a CTLA-4 antagonist, e.g., an anti-CTLA-4 antagonist antibody (e.g., ipilimumab (YERVOY®)), or an agent that increases or activates one or more immune co-stimulatory receptors (e.g., one or more immune co-stimulatory receptors selected from CD226, OX-40, CD28, CD27, CD137, HVEM, and/or GITR), such as an OX-40 agonist, e.g., an OX-40 agonist antibody), a chemotherapeutic agent, a cytotoxic agent, a growth inhibitory agent, a radiotherapy/radiation therapy, and/or an anti-hormonal agent, such as those recited herein above).

In another aspect, the invention provides methods for selecting a therapy for a subject or population of subjects having a cancer (e.g., cervical cancer, e.g., Stage IVB, metastatic, recurrent, or persistent cervical cancer, e.g., a metastatic and/or recurrent PD-L1-positive cervical carcinoma) by obtaining a tumor sample (e.g., a biopsy) from the subject or population of subjects, detecting the protein expression level of PD-L1 in the tumor sample by an IHC assay using an anti-PD-L1 antibody suitable for staining, and identifying the subject or population of subjects as one who is likely to benefit from a therapy comprising one or more dosing cycles of an anti-TIGIT antagonist antibody administered at a dose (e.g., a fixed dose) of 600 mg every three weeks and atezolizumab administered at a dose (e.g., a fixed dose) of 1200 mg every three weeks based on PD-L1 expression in the tumor sample having been detected, wherein the anti-TIGIT antagonist antibody comprises: a VH domain comprising the amino acid sequence of SEQ ID NO: 17 or 18, and a VL domain comprising the amino acid sequence of SEQ ID NO: 19. In some instances, the anti-TIGIT antagonist antibody has a VH domain having the amino acid sequence of SEQ ID NO: 17 and a VL domain having the amino acid sequence of SEQ ID NO: 19. In some instances, the anti-TIGIT antagonist antibody has a VH domain having the amino acid sequence of SEQ ID NO: 18 and a VL domain having the amino acid sequence of SEQ ID NO: 19. In some instances, the therapy comprises one or more dosing cycles of an anti-TIGIT antagonist antibody administered at a dose of 600 mg every three weeks and atezolizumab administered at a dose of 1680 mg every four weeks. In some instances, the therapy comprises one or more dosing cycles of an anti-TIGIT antagonist antibody administered at a dose of 600 mg every three weeks and atezolizumab administered at a dose of 840 mg every two weeks. In another aspect, the invention provides methods for selecting a therapy for a subject or population of subjects having a cancer (e.g., cervical cancer, e.g., Stage IVB, metastatic, recurrent, or persistent cervical cancer, e.g., a metastatic and/or recurrent PD-L1-positive cervical carcinoma) by obtaining a tumor sample (e.g., a biopsy) from the subject or population of subjects, detecting the protein expression level of PD-L1 in the tumor sample by an IHC assay using an anti-PD-L1 antibody suitable for staining, and identifying the subject or population of subjects as one who is likely to benefit from a therapy comprising one or more dosing cycles of tiragolumab administered at a dose of 600 mg every three weeks and atezolizumab administered at a dose of 1200 mg every three weeks based on PD-L1 expression in the tumor sample having been detected. In some instances, the method further includes administering to the identified subject or population of subjects the therapy. In some instances, the therapy may further include, or be administered in conjunction with (either separately or together), one or more additional anti-cancer therapeutic agent(s) (e.g., an immunomodulatory agent (e.g., an agent that decreases or inhibits one or more immune co-inhibitory receptors (e.g., one or more immune co-inhibitory receptors selected from TIGIT, PD-L1, PD-1, CTLA-4, LAG3, TIM3, BTLA, and/or VISTA), such as a CTLA-4 antagonist, e.g., an anti-CTLA-4 antagonist antibody (e.g., ipilimumab (YERVOY®)), or an agent that increases or activates one or more immune co-stimulatory receptors (e.g., one or more immune co-stimulatory receptors selected from CD226, OX-40, CD28, CD27, CD137, HVEM, and/or GITR), such as an OX-40 agonist, e.g., an OX-40 agonist antibody), a chemotherapeutic agent, a cytotoxic agent, a growth inhibitory agent, a radiotherapy/radiation therapy, and/or an anti-hormonal agent, such as those recited herein above).

In some instances, the invention provides methods for identifying a subject or population of subjects having a cancer (e.g., cervical cancer, e.g., Stage IVB, metastatic, recurrent, or persistent cervical cancer, e.g., a metastatic and/or recurrent PD-L1-positive cervical carcinoma) who may benefit from a therapy comprising an anti-TIGIT antagonist antibody and a PD-1 axis binding antagonist (e.g., an anti-PD-L1 antagonist antibody (e.g., atezolizumab) or an anti-PD-1 antagonist antibody (e.g., MDX-1106 (nivolumab) or MK-3475 (pembrolizumab, previously known as lambrolizumab))), by obtaining a tumor sample (e.g., a biopsy) from the subject or population of subjects, detecting the protein expression level of PD-L1 in the tumor sample by an IHC assay using an anti-PD-L1 antibody suitable for staining, and identifying the subject or population of subjects as one who is likely to benefit from a therapy comprising one or more dosing cycles of an anti-TIGIT antagonist antibody administered at a dose (e.g., a fixed dose) of 600 mg every three weeks and atezolizumab administered at a dose (e.g., a fixed dose) of 1200 mg every three weeks, wherein the anti-TIGIT antagonist antibody comprises: a VH domain comprising the amino acid sequence of SEQ ID NO: 17 or 18, and a VL domain comprising the amino acid sequence of SEQ ID NO: 19. In some instances, the anti-TIGIT antagonist antibody has a VH domain having the amino acid sequence of SEQ ID NO: 17 and a VL domain having the amino acid sequence of SEQ ID NO: 19. In some instances, the anti-TIGIT antagonist antibody has a VH domain having the amino acid sequence of SEQ ID NO: 18 and a VL domain having the amino acid sequence of SEQ ID NO: 19. In some instances, the therapy comprises one or more dosing cycles of an anti-TIGIT antagonist antibody administered at a dose of 600 mg every three weeks and atezolizumab administered at a dose of 1680 mg every four weeks. In some instances, the therapy comprises one or more dosing cycles of an anti-TIGIT antagonist antibody administered at a dose of 600 mg every three weeks and atezolizumab administered at a dose of 840 mg every two weeks. In some instances, the invention provides methods for identifying a subject or population of subjects having a cancer (e.g., cervical cancer, e.g., Stage IVB, metastatic, recurrent, or persistent cervical cancer, e.g., a metastatic and/or recurrent PD-L1-positive cervical carcinoma) who may benefit from a therapy comprising an anti-TIGIT antagonist antibody and a PD-1 axis binding antagonist (e.g., an anti-PD-L1 antagonist antibody (e.g., atezolizumab) or an anti-PD-1 antagonist antibody (e.g., MDX-1106 (nivolumab) or MK-3475 (pembrolizumab, previously known as lambrolizumab))), by obtaining a tumor sample (e.g., a biopsy) from the subject or population of subjects, detecting the protein expression level of PD-L1 in the tumor sample by an IHC assay using an anti-PD-L1 antibody suitable for staining, and identifying the subject or population of subjects as one who is likely to benefit from a therapy comprising one or more dosing cycles of tiragolumab administered at a dose of 600 mg every three weeks and atezolizumab administered at a dose of 1200 mg every three weeks. In some instances, the method further includes administering to the identified subject or population of subjects the therapy. In some instances, the therapy may further include, or be administered in conjunction with (either separately or together), one or more additional anti-cancer therapeutic agent(s) (e.g., an immunomodulatory agent (e.g., an agent that decreases or inhibits one or more immune co-inhibitory receptors (e.g., one or more immune co-inhibitory receptors selected from TIGIT, PD-L1, PD-1, CTLA-4, LAG3, TIM3, BTLA, and/or VISTA), such as a CTLA-4 antagonist, e.g., an anti-CTLA-4 antagonist antibody (e.g., ipilimumab (YERVOY®)), or an agent that increases or activates one or more immune co-stimulatory receptors (e.g., one or more immune co-stimulatory receptors selected from CD226, OX-40, CD28, CD27, CD137, HVEM, and/or GITR), such as an OX-40 agonist, e.g., an OX-40 agonist antibody), a chemotherapeutic agent, a cytotoxic agent, a growth inhibitory agent, a radiotherapy/radiation therapy, and/or an anti-hormonal agent, such as those recited herein above).

In some instances, the invention provides methods for identifying a subject or population of subjects having a cancer (e.g., cervical cancer, e.g., Stage IVB, metastatic, recurrent, or persistent cervical cancer, e.g., a metastatic and/or recurrent PD-L1-positive cervical carcinoma) who may benefit from a therapy comprising an anti-TIGIT antagonist antibody and a PD-1 axis binding antagonist (e.g., an anti-PD-L1 antagonist antibody (e.g., atezolizumab) or an anti-PD-1 antagonist antibody (e.g., MDX-1106 (nivolumab) or MK-3475 (pembrolizumab, previously known as lambrolizumab))), by obtaining a tumor sample (e.g., a biopsy) from the subject or population of subjects, detecting the protein expression level of PD-L1 in the tumor sample by an IHC assay using an anti-PD-L1 antibody suitable for staining, and identifying the subject or population of subjects as one who is likely to benefit from a therapy comprising one or more dosing cycles of an anti-TIGIT antagonist antibody administered at a dose of 600 mg every three weeks and atezolizumab administered at a dose of 1200 mg every three weeks, wherein the anti-TIGIT antagonist antibody comprises: a VH domain comprising the amino acid sequence of SEQ ID NO: 17 or 18, and a VL domain comprising the amino acid sequence of SEQ ID NO: 19. In some instances, the anti-TIGIT antagonist antibody has a VH domain having the amino acid sequence of SEQ ID NO: 17 and a VL domain having the amino acid sequence of SEQ ID NO: 19. In some instances, the anti-TIGIT antagonist antibody has a VH domain having the amino acid sequence of SEQ ID NO: 18 and a VL domain having the amino acid sequence of SEQ ID NO: 19. In some instances, the therapy comprises one or more dosing cycles of an anti-TIGIT antagonist antibody administered at a dose of 600 mg every three weeks and atezolizumab administered at a dose of 1680 mg every four weeks. In some instances, the therapy comprises one or more dosing cycles of an anti-TIGIT antagonist antibody administered at a dose of 600 mg every three weeks and atezolizumab administered at a dose of 840 mg every two weeks. In some instances, the invention provides methods for identifying a subject or population of subjects having a cancer (e.g., cervical cancer, e.g., Stage IVB, metastatic, recurrent, or persistent cervical cancer, e.g., a metastatic and/or recurrent PD-L1-positive cervical carcinoma) who may benefit from a therapy comprising an anti-TIGIT antagonist antibody and a PD-1 axis binding antagonist (e.g., an anti-PD-L1 antagonist antibody (e.g., atezolizumab) or an anti-PD-1 antagonist antibody (e.g., MDX-1106 (nivolumab) or MK-3475 (pembrolizumab, previously known as lambrolizumab))), by obtaining a tumor sample (e.g., a biopsy) from the subject or population of subjects, detecting the protein expression level of PD-L1 in the tumor sample by an IHC assay using an anti-PD-L1 antibody suitable for staining, and identifying the subject or population of subjects as one who is likely to benefit from a therapy comprising one or more dosing cycles of tiragolumab administered at a dose of 600 mg every three weeks and atezolizumab administered at a dose of 1200 mg every three weeks. In some instances, the method further includes administering to the identified subject or population of subjects the therapy. In some instances, the therapy may further include, or be administered in conjunction with (either separately or together), one or more additional anti-cancer therapeutic agent(s) (e.g., an immunomodulatory agent (e.g., an agent that decreases or inhibits one or more immune co-inhibitory receptors (e.g., one or more immune co-inhibitory receptors selected from TIGIT, PD-L1, PD-1, CTLA-4, LAG3, TIM3, BTLA, and/or VISTA), such as a CTLA-4 antagonist, e.g., an anti-CTLA-4 antagonist antibody (e.g., ipilimumab (YERVOY®)), or an agent that increases or activates one or more immune co-stimulatory receptors (e.g., one or more immune co-stimulatory receptors selected from CD226, OX-40, CD28, CD27, CD137, HVEM, and/or GITR), such as an OX-40 agonist, e.g., an OX-40 agonist antibody), a chemotherapeutic agent, a cytotoxic agent, a growth inhibitory agent, a radiotherapy/radiation therapy, and/or an anti-hormonal agent, such as those recited herein above).

In some instances, the invention provides methods for identifying a subject or population of subjects having a cancer (e.g., cervical cancer, e.g., Stage IVB, metastatic, recurrent, or persistent cervical cancer, e.g., a metastatic and/or recurrent PD-L1-positive cervical carcinoma) who may benefit from a therapy comprising an anti-TIGIT antagonist antibody and a PD-1 axis binding antagonist (e.g., an anti-PD-L1 antagonist antibody (e.g., atezolizumab) or an anti-PD-1 antagonist antibody (e.g., MDX-1106 (nivolumab) or MK-3475 (pembrolizumab, previously known as lambrolizumab))), by obtaining a tumor sample (e.g., a biopsy) from the subject or population of subjects, detecting the protein expression level of PD-L1 in the tumor sample by an IHC assay using an anti-PD-L1 antibody suitable for staining, and identifying the subject or population of subjects as one who is likely to benefit from a therapy comprising one or more dosing cycles of an anti-TIGIT antagonist antibody administered at a dose of 600 mg every three weeks and atezolizumab administered at a dose of 1200 mg every three weeks, wherein the anti-TIGIT antagonist antibody comprises: a VH domain comprising the amino acid sequence of SEQ ID NO: 17 or 18, and a VL domain comprising the amino acid sequence of SEQ ID NO: 19. In some instances, the anti-TIGIT antagonist antibody has a VH domain having the amino acid sequence of SEQ ID NO: 17 and a VL domain having the amino acid sequence of SEQ ID NO: 19. In some instances, the anti-TIGIT antagonist antibody has a VH domain having the amino acid sequence of SEQ ID NO: 18 and a VL domain having the amino acid sequence of SEQ ID NO: 19. In some instances, the therapy comprises one or more dosing cycles of an anti-TIGIT antagonist antibody administered at a dose of 600 mg every three weeks and atezolizumab administered at a dose of 1680 mg every four weeks. In some instances, the therapy comprises one or more dosing cycles of an anti-TIGIT antagonist antibody administered at a dose of 600 mg every three weeks and atezolizumab administered at a dose of 840 mg every two weeks. In some instances, the invention provides methods for identifying a subject or population of subjects having a cancer (e.g., cervical cancer, e.g., Stage IVB, metastatic, recurrent, or persistent cervical cancer, e.g., a metastatic and/or recurrent PD-L1-positive cervical carcinoma) who may benefit from a therapy comprising an anti-TIGIT antagonist antibody and a PD-1 axis binding antagonist (e.g., an anti-PD-L1 antagonist antibody (e.g., atezolizumab) or an anti-PD-1 antagonist antibody (e.g., MDX-1106 (nivolumab) or MK-3475 (pembrolizumab, previously known as lambrolizumab))), by obtaining a tumor sample (e.g., a biopsy) from the subject or population of subjects, detecting the protein expression level of PD-L1 in the tumor sample by an IHC assay using an anti-PD-L1 antibody suitable for staining, and identifying the subject or population of subjects as one who is likely to benefit from a therapy comprising one or more dosing cycles of tiragolumab administered at a dose of 600 mg every three weeks and atezolizumab administered at a dose of 1200 mg every three weeks. In some instances, the method further includes administering to the identified subject or population of subjects the therapy. In some instances, the therapy may further include, or be administered in conjunction with (either separately or together), one or more additional anti-cancer therapeutic agent(s) (e.g., an immunomodulatory agent (e.g., an agent that decreases or inhibits one or more immune co-inhibitory receptors (e.g., one or more immune co-inhibitory receptors selected from TIGIT, PD-L1, PD-1, CTLA-4, LAG3, TIM3, BTLA, and/or VISTA), such as a CTLA-4 antagonist, e.g., an anti-CTLA-4 antagonist antibody (e.g., ipilimumab (YERVOY®)), or an agent that increases or activates one or more immune co-stimulatory receptors (e.g., one or more immune co-stimulatory receptors selected from CD226, OX-40, CD28, CD27, CD137, HVEM, and/or GITR), such as an OX-40 agonist, e.g., an OX-40 agonist antibody), a chemotherapeutic agent, a cytotoxic agent, a growth inhibitory agent, a radiotherapy/radiation therapy, and/or an anti-hormonal agent, such as those recited herein above).

In some instances, the invention provides methods for assessing responsiveness of a subject or population of subjects having a cancer (e.g., cervical cancer, e.g., Stage IVB, metastatic, recurrent, or persistent cervical cancer, e.g., a metastatic and/or recurrent PD-L1-positive cervical carcinoma) to a therapy comprising an anti-TIGIT antagonist antibody and a PD-1 axis binding antagonist (e.g., an anti-PD-L1 antagonist antibody (e.g., atezolizumab) or an anti-PD-1 antagonist antibody (e.g., MDX-1106 (nivolumab) or MK-3475 (pembrolizumab, previously known as lambrolizumab))), by obtaining a tumor sample (e.g., a biopsy) from the subject or population of subjects, detecting the protein expression level of PD-L1 in the tumor sample by an IHC assay using an anti-PD-L1 antibody suitable for staining, and identifying the subject or population of subjects as one who is likely to benefit from a therapy comprising one or more dosing cycles of an anti-TIGIT antagonist antibody administered at a dose of 600 mg every three weeks and atezolizumab administered at a dose of 1200 mg every three weeks, wherein the anti-TIGIT antagonist antibody comprises: a VH domain comprising the amino acid sequence of SEQ ID NO: 17 or 18, and a VL domain comprising the amino acid sequence of SEQ ID NO: 19. In some instances, the anti-TIGIT antagonist antibody has a VH domain having the amino acid sequence of SEQ ID NO: 17 and a VL domain having the amino acid sequence of SEQ ID NO: 19. In some instances, the anti-TIGIT antagonist antibody has a VH domain having the amino acid sequence of SEQ ID NO: 18 and a VL domain having the amino acid sequence of SEQ ID NO: 19. In some instances, the therapy comprises one or more dosing cycles of an anti-TIGIT antagonist antibody administered at a dose of 600 mg every three weeks and atezolizumab administered at a dose of 1680 mg every four weeks. In some instances, the therapy comprises one or more dosing cycles of an anti-TIGIT antagonist antibody administered at a dose of 600 mg every three weeks and atezolizumab administered at a dose of 840 mg every two weeks. In some instances, the invention provides methods for assessing responsiveness of a subject or population of subjects having a cancer (e.g., cervical cancer, e.g., Stage IVB, metastatic, recurrent, or persistent cervical cancer, e.g., a metastatic and/or recurrent PD-L1-positive cervical carcinoma) to a therapy comprising an anti-TIGIT antagonist antibody and a PD-1 axis binding antagonist (e.g., an anti-PD-L1 antagonist antibody (e.g., atezolizumab) or an anti-PD-1 antagonist antibody (e.g., MDX-1106 (nivolumab) or MK-3475 (pembrolizumab, previously known as lambrolizumab))), by obtaining a tumor sample (e.g., a biopsy) from the subject or population of subjects, detecting the protein expression level of PD-L1 in the tumor sample by an IHC assay using an anti-PD-L1 antibody suitable for staining, and identifying the subject or population of subjects as one who is likely to benefit from a therapy comprising one or more dosing cycles of tiragolumab administered at a dose of 600 mg every three weeks and atezolizumab administered at a dose of 1200 mg every three weeks. In some instances, the method further includes administering to the identified subject or population of subjects the therapy. In some instances, the therapy may further include, or be administered in conjunction with (either separately or together), one or more additional anti-cancer therapeutic agent(s) (e.g., an immunomodulatory agent (e.g., an agent that decreases or inhibits one or more immune co-inhibitory receptors (e.g., one or more immune co-inhibitory receptors selected from TIGIT, PD-L1, PD-1, CTLA-4, LAG3, TIM3, BTLA, and/or VISTA), such as a CTLA-4 antagonist, e.g., an anti-CTLA-4 antagonist antibody (e.g., ipilimumab (YERVOY®)), or an agent that increases or activates one or more immune co-stimulatory receptors (e.g., one or more immune co-stimulatory receptors selected from CD226, OX-40, CD28, CD27, CD137, HVEM, and/or GITR), such as an OX-40 agonist, e.g., an OX-40 agonist antibody), a chemotherapeutic agent, a cytotoxic agent, a growth inhibitory agent, a radiotherapy/radiation therapy, and/or an anti-hormonal agent, such as those recited herein above).

In some instances, the invention provides methods for assessing responsiveness of a subject or population of subjects having a cancer (e.g., cervical cancer, e.g., Stage IVB, metastatic, recurrent, or persistent cervical cancer, e.g., a metastatic and/or recurrent PD-L1-positive cervical carcinoma) to a therapy comprising an anti-TIGIT antagonist antibody and a PD-1 axis binding antagonist (e.g., an anti-PD-L1 antagonist antibody (e.g., atezolizumab) or an anti-PD-1 antagonist antibody (e.g., MDX-1106 (nivolumab) or MK-3475 (pembrolizumab, previously known as lambrolizumab))), by obtaining a tumor sample (e.g., a biopsy) from the subject or population of subjects, detecting the protein expression level of PD-L1 in the tumor sample by an IHC assay using an anti-PD-L1 antibody suitable for staining, and identifying the subject or population of subjects as one who is likely to benefit from a therapy comprising one or more dosing cycles of an anti-TIGIT antagonist antibody administered at a dose of 600 mg every three weeks and atezolizumab administered at a dose of 1200 mg every three weeks, wherein the anti-TIGIT antagonist antibody comprises: a VH domain comprising the amino acid sequence of SEQ ID NO: 17 or 18, and a VL domain comprising the amino acid sequence of SEQ ID NO: 19. In some instances, the anti-TIGIT antagonist antibody has a VH domain having the amino acid sequence of SEQ ID NO: 17 and a VL domain having the amino acid sequence of SEQ ID NO: 19. In some instances, the anti-TIGIT antagonist antibody has a VH domain having the amino acid sequence of SEQ ID NO: 18 and a VL domain having the amino acid sequence of SEQ ID NO: 19. In some instances, the therapy comprises one or more dosing cycles of an anti-TIGIT antagonist antibody administered at a dose of 600 mg every three weeks and atezolizumab administered at a dose of 1680 mg every four weeks. In some instances, the therapy comprises one or more dosing cycles of an anti-TIGIT antagonist antibody administered at a dose of 600 mg every three weeks and atezolizumab administered at a dose of 840 mg every two weeks. In some instances, the invention provides methods for assessing responsiveness of a subject or population of subjects having a cancer (e.g., cervical cancer, e.g., Stage IVB, metastatic, recurrent, or persistent cervical cancer, e.g., a metastatic and/or recurrent PD-L1-positive cervical carcinoma) to a therapy comprising an anti-TIGIT antagonist antibody and a PD-1 axis binding antagonist (e.g., an anti-PD-L1 antagonist antibody (e.g., atezolizumab) or an anti-PD-1 antagonist antibody (e.g., MDX-1106 (nivolumab) or MK-3475 (pembrolizumab, previously known as lambrolizumab))), by obtaining a tumor sample (e.g., a biopsy) from the subject or population of subjects, detecting the protein expression level of PD-L1 in the tumor sample by an IHC assay using an anti-PD-L1 antibody suitable for staining, and identifying the subject or population of subjects as one who is likely to benefit from a therapy comprising one or more dosing cycles of tiragolumab administered at a dose of 600 mg every three weeks and atezolizumab administered at a dose of 1200 mg every three weeks. In some instances, the method further includes administering to the identified subject or population of subjects the therapy. In some instances, the therapy may further include, or be administered in conjunction with (either separately or together), one or more additional anti-cancer therapeutic agent(s) (e.g., an immunomodulatory agent (e.g., an agent that decreases or inhibits one or more immune co-inhibitory receptors (e.g., one or more immune co-inhibitory receptors selected from TIGIT, PD-L1, PD-1, CTLA-4, LAG3, TIM3, BTLA, and/or VISTA), such as a CTLA-4 antagonist, e.g., an anti-CTLA-4 antagonist antibody (e.g., ipilimumab (YERVOY®)), or an agent that increases or activates one or more immune co-stimulatory receptors (e.g., one or more immune co-stimulatory receptors selected from CD226, OX-40, CD28, CD27, CD137, HVEM, and/or GITR), such as an OX-40 agonist, e.g., an OX-40 agonist antibody), a chemotherapeutic agent, a cytotoxic agent, a growth inhibitory agent, a radiotherapy/radiation therapy, and/or an anti-hormonal agent, such as those recited herein above).

In some instances, the invention provides methods assessing responsiveness of a subject or population of subjects having a cancer (e.g., cervical cancer, e.g., Stage IVB, metastatic, recurrent, or persistent cervical cancer, e.g., a metastatic and/or recurrent PD-L1-positive cervical carcinoma) to a therapy comprising an anti-TIGIT antagonist antibody and a PD-1 axis binding antagonist (e.g., an anti-PD-L1 antagonist antibody (e.g., atezolizumab) or an anti-PD-1 antagonist antibody (e.g., MDX-1106 (nivolumab) or MK-3475 (pembrolizumab, previously known as lambrolizumab))), by obtaining a tumor sample (e.g., a biopsy) from the subject or population of subjects, detecting the protein expression level of PD-L1 in the tumor sample by an IHC assay using an anti-PD-L1 antibody suitable for staining, and identifying the subject or population of subjects as one who is likely to benefit from a therapy comprising one or more dosing cycles of an anti-TIGIT antagonist antibody administered at a dose of 600 mg every three weeks and atezolizumab administered at a dose of 1200 mg every three weeks, wherein the anti-TIGIT antagonist antibody comprises: a VH domain comprising the amino acid sequence of SEQ ID NO: 17 or 18, and a VL domain comprising the amino acid sequence of SEQ ID NO: 19. In some instances, the anti-TIGIT antagonist antibody has a VH domain having the amino acid sequence of SEQ ID NO: 17 and a VL domain having the amino acid sequence of SEQ ID NO: 19. In some instances, the anti-TIGIT antagonist antibody has a VH domain having the amino acid sequence of SEQ ID NO: 18 and a VL domain having the amino acid sequence of SEQ ID NO: 19. In some instances, the therapy comprises one or more dosing cycles of an anti-TIGIT antagonist antibody administered at a dose of 600 mg every three weeks and atezolizumab administered at a dose of 1680 mg every four weeks. In some instances, the therapy comprises one or more dosing cycles of an anti-TIGIT antagonist antibody administered at a dose of 600 mg every three weeks and atezolizumab administered at a dose of 840 mg every two weeks. In some instances, the invention provides methods assessing responsiveness of a subject or population of subjects having a cancer (e.g., cervical cancer, e.g., Stage IVB, metastatic, recurrent, or persistent cervical cancer, e.g., a metastatic and/or recurrent PD-L1-positive cervical carcinoma) to a therapy comprising an anti-TIGIT antagonist antibody and a PD-1 axis binding antagonist (e.g., an anti-PD-L1 antagonist antibody (e.g., atezolizumab) or an anti-PD-1 antagonist antibody (e.g., MDX-1106 (nivolumab) or MK-3475 (pembrolizumab, previously known as lambrolizumab))), by obtaining a tumor sample (e.g., a biopsy) from the subject or population of subjects, detecting the protein expression level of PD-L1 in the tumor sample by an IHC assay using an anti-PD-L1 antibody suitable for staining, and identifying the subject or population of subjects as one who is likely to benefit from a therapy comprising one or more dosing cycles of tiragolumab administered at a dose of 600 mg every three weeks and atezolizumab administered at a dose of 1200 mg every three weeks. In some instances, the method further includes administering to the identified subject or population of subjects the therapy. In some instances, the therapy may further include, or be administered in conjunction with (either separately or together), one or more additional anti-cancer therapeutic agent(s) (e.g., an immunomodulatory agent (e.g., an agent that decreases or inhibits one or more immune co-inhibitory receptors (e.g., one or more immune co-inhibitory receptors selected from TIGIT, PD-L1, PD-1, CTLA-4, LAG3, TIM3, BTLA, and/or VISTA), such as a CTLA-4 antagonist, e.g., an anti-CTLA-4 antagonist antibody (e.g., ipilimumab (YERVOY®)), or an agent that increases or activates one or more immune co-stimulatory receptors (e.g., one or more immune co-stimulatory receptors selected from CD226, OX-40, CD28, CD27, CD137, HVEM, and/or GITR), such as an OX-40 agonist, e.g., an OX-40 agonist antibody), a chemotherapeutic agent, a cytotoxic agent, a growth inhibitory agent, a radiotherapy/radiation therapy, and/or an anti-hormonal agent, such as those recited herein above).

In some instances, the invention provides methods for optimizing a therapy comprising an anti-TIGIT antagonist antibody and a PD-1 axis binding antagonist (e.g., an anti-PD-L1 antagonist antibody (e.g., atezolizumab) or an anti-PD-1 antagonist antibody (e.g., MDX-1106 (nivolumab) or MK-3475 (pembrolizumab, previously known as lambrolizumab))) in a subject or population of subjects having a cancer (e.g., cervical cancer, e.g., Stage IVB, metastatic, recurrent, or persistent cervical cancer, e.g., a metastatic and/or recurrent PD-L1-positive cervical carcinoma), by obtaining a tumor sample (e.g., a biopsy) from the subject or population of subjects, detecting the protein expression level of PD-L1 in the tumor sample by an IHC assay using an anti-PD-L1 antibody suitable for staining, and identifying the subject or population of subjects as one who is likely to benefit from a therapy comprising one or more dosing cycles of an anti-TIGIT antagonist antibody administered at a dose of 600 mg every three weeks and atezolizumab administered at a dose of 1200 mg every three weeks, wherein the anti-TIGIT antagonist antibody comprises: a VH domain comprising the amino acid sequence of SEQ ID NO: 17 or 18, and a VL domain comprising the amino acid sequence of SEQ ID NO: 19. In some instances, the anti-TIGIT antagonist antibody has a VH domain having the amino acid sequence of SEQ ID NO: 17 and a VL domain having the amino acid sequence of SEQ ID NO: 19. In some instances, the anti-TIGIT antagonist antibody has a VH domain having the amino acid sequence of SEQ ID NO: 18 and a VL domain having the amino acid sequence of SEQ ID NO: 19. In some instances, the therapy comprises one or more dosing cycles of an anti-TIGIT antagonist antibody administered at a dose of 600 mg every three weeks and atezolizumab administered at a dose of 1680 mg every four weeks. In some instances, the therapy comprises one or more dosing cycles of an anti-TIGIT antagonist antibody administered at a dose of 600 mg every three weeks and atezolizumab administered at a dose of 840 mg every two weeks. In some instances, the invention provides methods for optimizing a therapy comprising an anti-TIGIT antagonist antibody and a PD-1 axis binding antagonist (e.g., an anti-PD-L1 antagonist antibody (e.g., atezolizumab) or an anti-PD-1 antagonist antibody (e.g., MDX-1106 (nivolumab) or MK-3475 (pembrolizumab, previously known as lambrolizumab))) in a subject or population of subjects having a cancer (e.g., cervical cancer, e.g., Stage IVB, metastatic, recurrent, or persistent cervical cancer, e.g., a metastatic and/or recurrent PD-L1-positive cervical carcinoma), by obtaining a tumor sample (e.g., a biopsy) from the subject or population of subjects, detecting the protein expression level of PD-L1 in the tumor sample by an IHC assay using an anti-PD-L1 antibody suitable for staining, and identifying the subject or population of subjects as one who is likely to benefit from a therapy comprising one or more dosing cycles of tiragolumab administered at a dose of 600 mg every three weeks and atezolizumab administered at a dose of 1200 mg every three weeks. In some instances, the method further includes administering to the identified subject or population of subjects the therapy. In some instances, the therapy may further include, or be administered in conjunction with (either separately or together), one or more additional anti-cancer therapeutic agent(s) (e.g., an immunomodulatory agent (e.g., an agent that decreases or inhibits one or more immune co-inhibitory receptors (e.g., one or more immune co-inhibitory receptors selected from TIGIT, PD-L1, PD-1, CTLA-4, LAG3, TIM3, BTLA, and/or VISTA), such as a CTLA-4 antagonist, e.g., an anti-CTLA-4 antagonist antibody (e.g., ipilimumab (YERVOY®)), or an agent that increases or activates one or more immune co-stimulatory receptors (e.g., one or more immune co-stimulatory receptors selected from CD226, OX-40, CD28, CD27, CD137, HVEM, and/or GITR), such as an OX-40 agonist, e.g., an OX-40 agonist antibody), a chemotherapeutic agent, a cytotoxic agent, a growth inhibitory agent, a radiotherapy/radiation therapy, and/or an anti-hormonal agent, such as those recited herein above).

In some instances, the invention provides methods for optimizing a therapy comprising an anti-TIGIT antagonist antibody and a PD-1 axis binding antagonist (e.g., an anti-PD-L1 antagonist antibody (e.g., atezolizumab) or an anti-PD-1 antagonist antibody (e.g., MDX-1106 (nivolumab) or MK-3475 (pembrolizumab, previously known as lambrolizumab))) in a subject or population of subjects having a cancer (e.g., cervical cancer, e.g., Stage IVB, metastatic, recurrent, or persistent cervical cancer, e.g., a metastatic and/or recurrent PD-L1-positive cervical carcinoma) by obtaining a tumor sample (e.g., a biopsy) from the subject or population of subjects, detecting the protein expression level of PD-L1 in the tumor sample by an IHC assay using an anti-PD-L1 antibody suitable for staining, and identifying the subject or population of subjects as one who is likely to benefit from a therapy comprising one or more dosing cycles of an anti-TIGIT antagonist antibody administered at a dose of 600 mg every three weeks and atezolizumab administered at a dose of 1200 mg every three weeks, wherein the anti-TIGIT antagonist antibody comprises: a VH domain comprising the amino acid sequence of SEQ ID NO: 17 or 18, and a VL domain comprising the amino acid sequence of SEQ ID NO: 19. In some instances, the anti-TIGIT antagonist antibody has a VH domain having the amino acid sequence of SEQ ID NO: 17 and a VL domain having the amino acid sequence of SEQ ID NO: 19. In some instances, the anti-TIGIT antagonist antibody has a VH domain having the amino acid sequence of SEQ ID NO: 18 and a VL domain having the amino acid sequence of SEQ ID NO: 19. In some instances, the therapy comprises one or more dosing cycles of an anti-TIGIT antagonist antibody administered at a dose of 600 mg every three weeks and atezolizumab administered at a dose of 1680 mg every four weeks. In some instances, the therapy comprises one or more dosing cycles of an anti-TIGIT antagonist antibody administered at a dose of 600 mg every three weeks and atezolizumab administered at a dose of 840 mg every two weeks. In some instances, the invention provides methods for optimizing a therapy comprising an anti-TIGIT antagonist antibody and a PD-1 axis binding antagonist (e.g., an anti-PD-L1 antagonist antibody (e.g., atezolizumab) or an anti-PD-1 antagonist antibody (e.g., MDX-1106 (nivolumab) or MK-3475 (pembrolizumab, previously known as lambrolizumab))) in a subject or population of subjects having a cancer (e.g., cervical cancer, e.g., Stage IVB, metastatic, recurrent, or persistent cervical cancer, e.g., a metastatic and/or recurrent PD-L1-positive cervical carcinoma) by obtaining a tumor sample (e.g., a biopsy) from the subject or population of subjects, detecting the protein expression level of PD-L1 in the tumor sample by an IHC assay using an anti-PD-L1 antibody suitable for staining, and identifying the subject or population of subjects as one who is likely to benefit from a therapy comprising one or more dosing cycles of tiragolumab administered at a dose of 600 mg every three weeks and atezolizumab administered at a dose of 1200 mg every three weeks. In some instances, the method further includes administering to the identified subject or population of subjects the therapy. In some instances, the therapy may further include, or be administered in conjunction with (either separately or together), one or more additional anti-cancer therapeutic agent(s) (e.g., an immunomodulatory agent (e.g., an agent that decreases or inhibits one or more immune co-inhibitory receptors (e.g., one or more immune co-inhibitory receptors selected from TIGIT, PD-L1, PD-1, CTLA-4, LAG3, TIM3, BTLA, and/or VISTA), such as a CTLA-4 antagonist, e.g., an anti-CTLA-4 antagonist antibody (e.g., ipilimumab (YERVOY®)), or an agent that increases or activates one or more immune co-stimulatory receptors (e.g., one or more immune co-stimulatory receptors selected from CD226, OX-40, CD28, CD27, CD137, HVEM, and/or GITR), such as an OX-40 agonist, e.g., an OX-40 agonist antibody), a chemotherapeutic agent, a cytotoxic agent, a growth inhibitory agent, a radiotherapy/radiation therapy, and/or an anti-hormonal agent, such as those recited herein above).

In some instances, the invention provides methods optimizing a therapy comprising an anti-TIGIT antagonist antibody and a PD-1 axis binding antagonist (e.g., an anti-PD-L1 antagonist antibody (e.g., atezolizumab) or an anti-PD-1 antagonist antibody (e.g., MDX-1106 (nivolumab) or MK-3475 (pembrolizumab, previously known as lambrolizumab))) in a subject or population of subjects having a cancer (e.g., cervical cancer, e.g., Stage IVB, metastatic, recurrent, or persistent cervical cancer, e.g., a metastatic and/or recurrent PD-L1-positive cervical carcinoma) by obtaining a tumor sample (e.g., a biopsy) from the subject or population of subjects, detecting the protein expression level of PD-L1 in the tumor sample by an IHC assay using an anti-PD-L1 antibody suitable for staining, and identifying the subject or population of subjects as one who is likely to benefit from a therapy comprising one or more dosing cycles of an anti-TIGIT antagonist antibody administered at a dose of 600 mg every three weeks and atezolizumab administered at a dose of 1200 mg every three weeks, wherein the anti-TIGIT antagonist antibody comprises: a VH domain comprising the amino acid sequence of SEQ ID NO: 17 or 18, and a VL domain comprising the amino acid sequence of SEQ ID NO: 19. In some instances, the anti-TIGIT antagonist antibody has a VH domain having the amino acid sequence of SEQ ID NO: 17 and a VL domain having the amino acid sequence of SEQ ID NO: 19. In some instances, the anti-TIGIT antagonist antibody has a VH domain having the amino acid sequence of SEQ ID NO: 18 and a VL domain having the amino acid sequence of SEQ ID NO: 19. In some instances, the therapy comprises one or more dosing cycles of an anti-TIGIT antagonist antibody administered at a dose of 600 mg every three weeks and atezolizumab administered at a dose of 1680 mg every four weeks. In some instances, the therapy comprises one or more dosing cycles of an anti-TIGIT antagonist antibody administered at a dose of 600 mg every three weeks and atezolizumab administered at a dose of 840 mg every two weeks. In some instances, the invention provides methods for optimizing a therapy comprising an anti-TIGIT antagonist antibody and a PD-1 axis binding antagonist (e.g., an anti-PD-L1 antagonist antibody (e.g., atezolizumab) or an anti-PD-1 antagonist antibody (e.g., MDX-1106 (nivolumab) or MK-3475 (pembrolizumab, previously known as lambrolizumab))) in a subject or population of subjects having a cancer (e.g., cervical cancer, e.g., Stage IVB, metastatic, recurrent, or persistent cervical cancer, e.g., a metastatic and/or recurrent PD-L1-positive cervical carcinoma) by obtaining a tumor sample (e.g., a biopsy) from the subject or population of subjects, detecting the protein expression level of PD-L1 in the tumor sample by an IHC assay using an anti-PD-L1 antibody suitable for staining, and identifying the subject or population of subjects as one who is likely to benefit from a therapy comprising one or more dosing cycles of tiragolumab administered at a dose of 600 mg every three weeks and atezolizumab administered at a dose of 1200 mg every three weeks. In some instances, the method further includes administering to the identified subject or population of subjects the therapy. In some instances, the therapy may further include, or be administered in conjunction with (either separately or together), one or more additional anti-cancer therapeutic agent(s) (e.g., an immunomodulatory agent (e.g., an agent that decreases or inhibits one or more immune co-inhibitory receptors (e.g., one or more immune co-inhibitory receptors selected from TIGIT, PD-L1, PD-1, CTLA-4, LAG3, TIM3, BTLA, and/or VISTA), such as a CTLA-4 antagonist, e.g., an anti-CTLA-4 antagonist antibody (e.g., ipilimumab (YERVOY®)), or an agent that increases or activates one or more immune co-stimulatory receptors (e.g., one or more immune co-stimulatory receptors selected from CD226, OX-40, CD28, CD27, CD137, HVEM, and/or GITR), such as an OX-40 agonist, e.g., an OX-40 agonist antibody), a chemotherapeutic agent, a cytotoxic agent, a growth inhibitory agent, a radiotherapy/radiation therapy, and/or an anti-hormonal agent, such as those recited herein above).

In some instances, the method further includes administering to the identified subject or population of subjects the therapy. In some instances, the therapy may further include, or be administered in conjunction with (either separately or together), one or more additional anti-cancer therapeutic agent(s) (e.g., an immunomodulatory agent (e.g., an agent that decreases or inhibits one or more immune co-inhibitory receptors (e.g., one or more immune co-inhibitory receptors selected from TIGIT, PD-L1, PD-1, CTLA-4, LAG3, TIM3, BTLA, and/or VISTA), such as a CTLA-4 antagonist, e.g., an anti-CTLA-4 antagonist antibody (e.g., ipilimumab (YERVOY®)), or an agent that increases or activates one or more immune co-stimulatory receptors (e.g., one or more immune co-stimulatory receptors selected from CD226, OX-40, CD28, CD27, CD137, HVEM, and/or GITR), such as an OX-40 agonist, e.g., an OX-40 agonist antibody), a chemotherapeutic agent, a cytotoxic agent, a growth inhibitory agent, a radiotherapy/radiation therapy, and/or an anti-hormonal agent, such as those recited herein above).

In some instances, the method further includes administering to the identified subject or population of subjects the therapy. In some instances, the therapy may further include, or be administered in conjunction with (either separately or together), one or more additional anti-cancer therapeutic agent(s) (e.g., an immunomodulatory agent (e.g., an agent that decreases or inhibits one or more immune co-inhibitory receptors (e.g., one or more immune co-inhibitory receptors selected from TIGIT, PD-L1, PD-1, CTLA-4, LAG3, TIM3, BTLA, and/or VISTA), such as a CTLA-4 antagonist, e.g., an anti-CTLA-4 antagonist antibody (e.g., ipilimumab (YERVOY®)), or an agent that increases or activates one or more immune co-stimulatory receptors (e.g., one or more immune co-stimulatory receptors selected from CD226, OX-40, CD28, CD27, CD137, HVEM, and/or GITR), such as an OX-40 agonist, e.g., an OX-40 agonist antibody), a chemotherapeutic agent, a cytotoxic agent, a growth inhibitory agent, a radiotherapy/radiation therapy, and/or an anti-hormonal agent, such as those recited herein above).

In some instances, in any of the diagnostic methods or uses described herein, the cervical cancer is a Stage IVB, metastatic, recurrent, or persistent cervical cancer. The cancer may be at an early or late stage.

D. Therapeutic Methods and Uses Relating to Breast Cancer

Breast Cancer

Breast cancer is the most frequent cancer diagnosed in women, with an estimated global incidence of 2.08 million new cases reported in 2018. Breast cancer accounts for approximately 15% (approximately 626,700 cases) of all cancer deaths and is the most common cause of cancer-related mortality in women, with a five-year survival rate of approximately 15% following metastatic diagnosis. The majority of patients are diagnosed with localized breast cancer; however, approximately 6% of patients present with de novo metastatic disease and between 10% and 40% of patients with localized breast cancer will relapse systemically. In the early stages of breast cancer (I-III; early breast cancer), the largely asymptomatic disease is usually operable and can be treated with curative intent.

Breast cancer is a heterogeneous disease encompassing about 15 different types of carcinomas, which for therapeutic reasons are further classified according to their estrogen receptor (ER), progesterone receptor (PR), and human epidermal growth factor receptor 2 (HER2) status. These subgroups have important implications for the choice of therapy, treatment outcomes, recurrence rate, and mortality risk. Triple-negative breast cancer (TNBC) is characterized by the lack of expression of ER, PR, and HER2. Overall, approximately 15%-20% of all breast cancers are classified as TNBC. Large-scale comprehensive genomic analyses have characterized the heterogeneous nature of TNBCs and their diverse gene expression patterns and underlying genomic changes, but these insights have not yet provided clear guidance for the identification of clinically effective targeted therapies currently under laboratory and clinical investigation.

TNBCs are more likely to have aggressive features such as a high proliferative rate. Patients with metastatic TNBC exhibit a particularly poor clinical outcome, generally with rapid progression and median OS rate of approximately 16 months. Despite recent improvements in treatment, the prognosis for patients with TNBC remains far from optimal and in fact has a five-year survival rate following metastatic diagnosis of approximately 15%.

Therefore, there is a high unmet need for improved medical intervention of TNBC, including early TNBC (eTNBC).

Methods and Uses for Treating Breast Cancer

In some instances, a subject or population of subjects receiving the anti-TIGIT antagonist antibody (e.g., an anti-TIGIT antagonist antibody as disclosed herein, e.g., tiragolumab), the PD-1 axis binding antagonist (e.g., anti-PD-L1 antagonist antibody, such as atezolizumab), and the chemotherapy (e.g., a taxane (e.g., nab-paclitaxel or paclitaxel)) is being treated for a breast cancer (e.g., HER2+breast cancer and TNBC). In some instances, the anti-TIGIT antagonist antibody is tiragolumab. In some instances, the PD-1 axis binding antagonist is an anti-PD-L1 antagonist antibody. In some instances, the anti-PD-L1 antagonist antibody is atezolizumab. In some instances, the chemotherapy is a taxane. In some instances, the taxane is nab-paclitaxel or paclitaxel. In some instances, the taxane is nab-paclitaxel. In some instances, the breast cancer is TNBC. In some instances, the TNBC is a PD-L1-positive TNBC. In some instances, the TNBC is unresectable locally advanced or metastatic. In some instances, the PD-L1-positive TNBC is unresectable locally advanced or metastatic. In some instances, the subject or population of subjects has not received prior systemic therapy for breast cancer. In some instances, the subject or population of subjects has not received prior systemic therapy for metastatic breast cancer.

Methods and Uses for Treating eTNBC

Provided herein are methods and uses for treating an early TNBC (eTNBC) in a subject or population of subjects comprising administering to the subject or population of subjects a dosing regimen comprising one or more dosing cycles of an effective amount of an anti-TIGIT antagonist antibody (e.g., an anti-TIGIT antagonist antibody as disclosed herein, e.g., tiragolumab) and an effective amount of a PD-1 axis binding antagonist (e.g., an anti-PD-L1 antagonist antibody (e.g., atezolizumab) or an anti-PD-1 antagonist antibody (e.g., MDX-1106 (nivolumab) or MK-3475 (pembrolizumab, previously known as lambrolizumab))), thereby treating the subject or population of subjects.

The present invention includes methods and uses involving administration of an effective amount of an anti-TIGIT antagonist antibody (e.g., an anti-TIGIT antagonist antibody as disclosed herein, e.g., tiragolumab) and a PD-1 axis binding antagonist (e.g., an anti-PD-L1 antagonist antibody, such as atezolizumab) to a subject or population of subjects in need thereof. In some instances, the anti-TIGIT antagonist antibody (e.g., an anti-TIGIT antagonist antibody as disclosed herein, e.g., tiragolumab) and PD-1 axis binding antagonist (e.g., an anti-PD-L1 antagonist antibody, such as atezolizumab) are administered with or without one or more chemotherapeutic agents (e.g., a platinum-based chemotherapeutic agent (e.g., carboplatin or cisplatin) and/or a non-platinum-based chemotherapeutic agent (e.g., an alkylating agent (e.g., cyclophosphamide), a taxane (e.g., paclitaxel, e.g., nab-paclitaxel), and/or a topoisomerase II inhibitor (e.g., doxorubicin))) to a subject or population of subjects in need thereof. In some instances, the method further comprises one or more surgeries (e.g., a mastectomy and/or an axillary lymph node surgery). In some instances, the surgery is performed after administering a dosing regimen including the anti-TIGIT antagonist antibody, the PD-1 axis binding antagonist, the one or more chemotherapeutic agents, and/or G-CSF or GM-CSF. In some instances, the surgery is a mastectomy and/or an axillary lymph node surgery. In some instances, the surgery is performed between two and six weeks (e.g., 2 weeks, 3 weeks, 4 weeks, 5 weeks, and 6 weeks) after the last dose of the dosing regimen.

In some instances, any of the methods and uses for treating an eTNBC in a subject or population of subjects can result in a pCR. In some instances, any of the methods and uses for treating an eTNBC in a subject or population of subjects can result in an increase in OS or event-free survival (EFS).

Dosing Regimens and Administration

The therapeutic methods and uses of the invention described herein include, in one aspect, administering to a subject or population of subjects having an eTNBC a treatment regimen comprising an effective amount of a PD-1 axis binding antagonist (e.g., an anti-PD-L1 antagonist antibody (e.g., atezolizumab) or an anti-PD-1 antagonist antibody (e.g., MDX-1106 (nivolumab) or MK-3475 (pembrolizumab, previously known as lambrolizumab))) and an anti-TIGIT antagonist antibody (e.g., tiragolumab). In some instances, the therapeutic methods and uses of the invention described herein include administering to a subject or population of subjects having an eTNBC a treatment regimen comprising an effective amount of a PD-1 axis binding antagonist (e.g., an anti-PD-L1 antagonist antibody (e.g., atezolizumab) or an anti-PD-1 antagonist antibody (e.g., MDX-1106 (nivolumab) or MK-3475 (pembrolizumab, previously known as lambrolizumab))) and an anti-TIGIT antagonist antibody (e.g., tiragolumab) with or without one or more chemotherapeutic agents (e.g., a platinum-based chemotherapeutic agent (e.g., carboplatin or cisplatin) and/or a non-platinum-based chemotherapeutic agent (e.g., an alkylating agent (e.g., cyclophosphamide), a taxane (e.g., paclitaxel, e.g., nab-paclitaxel), and/or a topoisomerase II inhibitor (e.g., doxorubicin))) and/or G-CSF or GM-CSF. In some aspects, the topoisomerase II inhibitor is doxorubicin. In some aspects, doxorubicin is administered at a dose of about 60 mg/m².

The pharmaceutical compositions described herein can be formulated for administration as described in Section III(K).

Dosing of Chemotherapeutic Agents

Therapeutically effective amounts of various chemotherapeutic agents are known in the art and contemplated in the present invention. In particular instances, one or more chemotherapeutic agents (e.g., a platinum-based chemotherapeutic agent (e.g., carboplatin or cisplatin) and/or one or more non-platinum-based chemotherapeutic agents (e.g., an alkylating agent (e.g., cyclophosphamide), a taxane (e.g., paclitaxel, e.g., nab-paclitaxel), and/or a topoisomerase II inhibitor (e.g., doxorubicin))) are administered according to the doses recited in Section III(K) herein.

Cancer Characterization and Selection

In any of the methods, uses, or compositions for use described herein, the cancer may be breast cancer. In some instances, the breast cancer is TNBC. In some instances, the TNBC is eTNBC. In some instances, the eTNBC is a T2-4d TNBC at presentation. In some instances, the eTNBC is a cT2-cT4, cN0-cN3, and cM0 TNBC at presentation. In some instances, the eTNBC is PD-L1-positive. In some instances, the eTNBC is PD-L1-negative. In some instances, the subject or population of subjects has not been previously treated for eTNBC. In some instances, the subject or population of subjects has had no prior systemic treatment for breast cancer (e.g., TNBC, e.g., eTNBC).

In some instances, in any of the methods, uses, or compositions for use described herein, the subject or population of subjects has a PD-L1 selected tumor (e.g., a proportion of tumor area occupied by PD-L1 expressing tumor-infiltrating immune cells (ICs) is greater than or equal to 1% in the tumor sample as determined by an IHC with the SP142 antibody). In some instances, the PD-L1 selected tumor is a tumor that has been determined to have a proportion of tumor area occupied by PD-L1 expressing ICs greater than or equal to 1% by an immunohistochemical (IHC) assay. In some instances, the IHC assay uses the anti-PD-L1 antibody SP142, SP263, 22C3, or 28-8. In some instances, the IHC assay uses anti-PD-L1 antibody SP142. In some instances, the ICs has been determined to be greater than, or equal to, 1% (e.g., as determined using the Ventana (SP142) PD-L1 IHC assay). In some instances, PD-L1 expression level is detected using anti-PD-L1 antibody 22C3. In some instances, the detectable expression level of PD-L1 is a combined positive score (CPS) of greater than or equal to 1 in a sample from the subject.

In some instances, in any of the methods, uses, or compositions for use described herein, a tumor sample obtained from the individual has a detectable nucleic acid expression level of PD-L1. In some instances, the detectable nucleic acid expression level of PD-L1 has been determined by RNA-seq, RT-qPCR, qPCR, multiplex qPCR or RT-qPCR, microarray analysis, SAGE, MassARRAY technique, ISH, or a combination thereof. In some instances, the sample is selected from the group consisting of a tissue sample, a whole blood sample, a serum sample, and a plasma sample. In some instances, the tissue sample is a tumor sample. In some instances, the tumor sample comprises tumor-infiltrating immune cells, tumor cells, stromal cells, and any combinations thereof.

In some instances, a tumor sample from the subject or population of subjects has been determined to be PD-L1-positive. In some instances, a tumor sample from the subject or population of subjects has been determined to be PD-L1-negative. In some instances, most tumor samples from the subject or population of subjects has been determined to be PD-L1-negative. In some instances, every tumor sample from the subject or population of subjects has been determined to be PD-L1-negative.

Responses to Treatment

In some embodiments of any of the methods described herein, a subject or population of subjects' response to the therapy can be characterized by one or more measures. In some embodiments, the treatment results in a CR or a PR.

In some instances, the treatment results in an increase in event-free survival of the subject or population of subjects, e.g., as compared to treatment with the PD-1 axis binding antagonist without the anti-TIGIT antagonist antibody or as compared to treatment with the anti-TIGIT antagonist antibody without the PD-1 axis binding antagonist. For example, in embodiments in which no chemotherapeutic agent is administered (e.g., only an anti-TIGIT antagonist antibody (e.g., tiragolumab) in combination with a PD-1 axis binding antagonist (e.g., atezolizumab) is administered), the treatment may result in an increase in event-free survival of the subject or population of subjects, e.g., as compared to treatment with the PD-1 axis binding antagonist without the anti-TIGIT antagonist antibody or as compared to treatment with the anti-TIGIT antagonist antibody without the PD-1 axis binding antagonist. In embodiments in which an anti-TIGIT antagonist antibody (e.g., tiragolumab) and a PD-1 axis binding antagonist (e.g., atezolizumab) are administered in combination with one or more chemotherapeutic agents (e.g., a platinum-based chemotherapeutic agent (e.g., carboplatin or cisplatin) and/or one or more non-platinum-based chemotherapeutic agents (e.g., an alkylating agent (e.g., cyclophosphamide), a taxane (e.g., paclitaxel, e.g., nab-paclitaxel), and/or a topoisomerase II inhibitor (e.g., doxorubicin))) and/or G-CSF or GM-CSF, the treatment may result in an increase in event-free survival of the subject or population of subjects, e.g., as compared to (i) treatment with the PD-1 axis binding antagonist and the one or more chemotherapeutic agents and/or G-CSF or GM-CSF without the anti-TIGIT antagonist antibody; (ii) as compared to treatment with the anti-TIGIT antagonist antibody and the one or more chemotherapeutic agents and/or G-CSF or GM-CSF without the PD-1 axis binding antagonist; and/or (iii) as compared to treatment with the PD-1 axis binding antagonist and the anti-TIGIT antagonist antibody without the one or more chemotherapeutic agents and/or G-CSF or GM-CSF.

In some instances, the treatment extends OS of the subject or population of subjects, e.g., as compared to treatment with the PD-1 axis binding antagonist without the anti-TIGIT antagonist antibody or as compared to treatment with the anti-TIGIT antagonist antibody without the PD-1 axis binding antagonist. For example, in embodiments in which no chemotherapeutic agent is administered (e.g., only an anti-TIGIT antagonist antibody (e.g., tiragolumab) in combination with a PD-1 axis binding antagonist (e.g., atezolizumab) is administered), the treatment may result in an increase in OS of the subject or population of subjects, e.g., as compared to treatment with the PD-1 axis binding antagonist without the anti-TIGIT antagonist antibody or as compared to treatment with the anti-TIGIT antagonist antibody without the PD-1 axis binding antagonist. In embodiments in which an anti-TIGIT antagonist antibody (e.g., tiragolumab) and a PD-1 axis binding antagonist (e.g., atezolizumab) are administered in combination with one or more chemotherapeutic agents (e.g., a platinum-based chemotherapeutic agent (e.g., carboplatin or cisplatin) and/or one or more non-platinum-based chemotherapeutic agents (e.g., an alkylating agent (e.g., cyclophosphamide), a taxane (e.g., paclitaxel, e.g., nab-paclitaxel), and/or a topoisomerase II inhibitor (e.g., doxorubicin))) and/or G-CSF or GM-CSF, the treatment may result in an increase in OS of the subject or population of subjects, e.g., as compared to (i) treatment with the PD-1 axis binding antagonist and the one or more chemotherapeutic agents and/or G-CSF or GM-CSF without the anti-TIGIT antagonist antibody; (ii) as compared to treatment with the anti-TIGIT antagonist antibody and the one or more chemotherapeutic agents and/or G-CSF or GM-CSF without the PD-1 axis binding antagonist; and/or (iii) as compared to treatment with the PD-1 axis binding antagonist and the anti-TIGIT antagonist antibody without the one or more chemotherapeutic agents and/or G-CSF or GM-CSF.

Event-free survival of the subject or population of subjects can be measured according to RECIST v1.1 criteria, as described in Eisenhauer et al., Eur. J. Cancer. 2009, 45:228-47. In some embodiments, EFS is measured as the period of time from the start of treatment to the first occurrence of disease progression as determined by RECIST v1.1 criteria. In some embodiments, EFS is measured as the time from the start of treatment to the time of death.

In some embodiments, a treatment described herein extends the EFS of the subject or population of subjects by at least about 2.4 months (e.g., by 2.4-120 months, by 2.5-100 months, by 3.0-80 months, by 4.0-60 months, by 5.0-48 months, by 6.0-36 months, by 8.0-24 months, or by 10-12 months, e.g., by at least about 2.4 months, 2.5 months, 2.6 months, 2.7 months, 2.8 months, 2.9 months, 3.0 months, 3.1 months, 3.2 months, 3.3 months, 3.4 months, 3.5 months, 3.6 months, 3.7 months, 3.8 months, 3.9 months, 4.0 months, 4.1 months, 4.2 months, 4.3 months, 4.4 months, 4.5 months, 4.6 months, 4.7 months, 4.8 months, 4.9 months, 5.0 months, 5.5 months, 6.0 months, 6.5 months, 7.0 months, 7.5 months, 8.0 months, 8.5 months, 9.0 months, 9.5 months, 10 months, 10.5 months, 11 months, 11.5 months, 12 months, 13 months, 14 months, 15 months, 16 months, 17 months, 18 months, 19 months, 20 months, 21 months, 22 months, 23 months, 24 months, 25 months, 26 months, 27 months, 28 months, 29 months, 30 months, 31 months, 32 months, 33 months, 34 months, 35 months, or 36 months). In some embodiments, the treatment extends the EFS of the subject or population of subjects by at least about 4 months (e.g., by 4-120 months, by 5-100 months, by 6-80 months, by 7-60 months, by 8-48 months, by 9-36 months, or by 10-24 months, e.g., by at least about 4.0 months, 4.1 months, 4.2 months, 4.3 months, 4.4 months, 4.5 months, 4.6 months, 4.7 months, 4.8 months, 4.9 months, 5.0 months, 5.5 months, 6.0 months, 6.5 months, 7.0 months, 7.5 months, 8.0 months, 8.5 months, 9.0 months, 9.5 months, 10 months, 10.5 months, 11 months, 11.5 months, 12 months, 13 months, 14 months, 15 months, 16 months, 17 months, 18 months, 19 months, 20 months, 21 months, 22 months, 23 months, 24 months, 25 months, 26 months, 27 months, 28 months, 29 months, 30 months, 31 months, 32 months, 33 months, 34 months, 35 months, or 36 months). In some embodiments, the treatment extends the EFS of the subject or population of subjects by at least about 2 months (e.g., by 2-120 months, by 3-100 months, by 4-80 months, by 6-60 months, by 8-48 months, by 9-36 months, or by 10-24 months, e.g., by at least about 2.0 months, 2.1 months, 2.2 months, 2.3 months, 2.4 months, 2.5 months, 2.6 months, 2.7 months, 2.8 months, 2.9 months, 3.0 months, 3.1 months, 3.2 months, 3.3 months, 3.4 months, 3.5 months, 3.6 months, 3.7 months, 3.8 months, 3.9 months, 4.0 months, 4.1 months, 4.2 months, 4.3 months, 4.4 months, 4.5 months, 4.6 months, 4.7 months, 4.8 months, 4.9 months, 5.0 months, 5.5 months, 6.0 months, 6.5 months, 7.0 months, 7.5 months, 8.0 months, 8.5 months, 9.0 months, 9.5 months, 10 months, 10.5 months, 11 months, 11.5 months, 12 months, 13 months, 14 months, 15 months, 16 months, 17 months, 18 months, 19 months, 20 months, 21 months, 22 months, 23 months, 24 months, 25 months, 26 months, 27 months, 28 months, 29 months, 30 months, 31 months, 32 months, 33 months, 34 months, 35 months, or 36 months).

In some embodiments, OS is measured as the period of time from the start of treatment to death. In some instances, the treatment extends the OS of the subject or population of subjects by at least about 2 months (e.g., by 2-120 months, by 3-110 months, by 4-100 months, by 5-80 months, by 6-60 months, by 7-48 months, by 8-36 months, or by 10-24 months, e.g., by at least about 2 months, 2.1 months, 2.2 months, 2.3 months, 2.4 months, 2.5 months, 2.6 months, 2.7 months, 2.8 months, 2.9 months, 3.0 months, 3.1 months, 3.2 months, 3.3 months, 3.4 months, 3.5 months, 3.6 months, 3.7 months, 3.8 months, 3.9 months, 4.0 months, 4.1 months, 4.2 months, 4.3 months, 4.4 months, 4.5 months, 4.6 months, 4.7 months, 4.8 months, 4.9 months, 5.0 months, 5.5 months, 6.0 months, 6.5 months, 7.0 months, 7.5 months, 8.0 months, 8.5 months, 9.0 months, 9.5 months, 10 months, 10.5 months, 11 months, 11.5 months, 12 months, 13 months, 14 months, 15 months, 16 months, 17 months, 18 months, 19 months, 20 months, 21 months, 22 months, 23 months, 24 months, 25 months, 26 months, 27 months, 28 months, 29 months, 30 months, 31 months, 32 months, 33 months, 34 months, 35 months, or 36 months). In some instances, the treatment extends the OS of the subject or population of subjects by at least about 3.3 months (e.g., by 3.3-120 months, by 4-100 months, by 5-80 months, by 6-60 months, by 7-48 months, by 8-36 months, or by 10-24 months, e.g., by at least about 3.3 months, 3.4 months, 3.5 months, 3.6 months, 3.7 months, 3.8 months, 3.9 months, 4.0 months, 4.1 months, 4.2 months, 4.3 months, 4.4 months, 4.5 months, 4.6 months, 4.7 months, 4.8 months, 4.9 months, 5.0 months, 5.5 months, 6.0 months, 6.5 months, 7.0 months, 7.5 months, 8.0 months, 8.5 months, 9.0 months, 9.5 months, 10 months, 10.5 months, 11 months, 11.5 months, 12 months, 13 months, 14 months, 15 months, 16 months, 17 months, 18 months, 19 months, 20 months, 21 months, 22 months, 23 months, 24 months, 25 months, 26 months, 27 months, 28 months, 29 months, 30 months, 31 months, 32 months, 33 months, 34 months, 35 months, or 36 months). In some instances, the treatment extends the OS of the subject or population of subjects by at least about 5.3 months (e.g., by 5.3-120, by 6-60 months, by 7-48 months, by 8-36 months, or by 10-24 months, e.g., by at least about 5.3 months, 5.5 months, 6.0 months, 6.5 months, 7.0 months, 7.5 months, 8.0 months, 8.5 months, 9.0 months, 9.5 months, 10 months, 10.5 months, 11 months, 11.5 months, 12 months, 13 months, 14 months, 15 months, 16 months, 17 months, 18 months, 19 months, 20 months, 21 months, 22 months, 23 months, 24 months, 25 months, 26 months, 27 months, 28 months, 29 months, 30 months, 31 months, 32 months, 33 months, 34 months, 35 months, or 36 months). In some embodiments, the treatment results in a median OS of the population of subjects of at least about 20 months (e.g., between about 20 months and about 36 months (e.g., 20 months, 21 months, 22 months, 23 months, 24 months, 25 months, 26 months, 27 months, 28 months, 29 months, 30 months, 31 months, 32 months, 33 months, 34 months, 35 months, or 36 months)). In some embodiments, the treatment results in a median OS of the population of subjects of about 25.0 months.

In some embodiments, the treatment results in an ORR of the population of subjects of at least about 53% (e.g., about 53% to about 100% (e.g., about 53%, 54%, 55%, 56%, 57%, 58%, 59%, 60%, 61%, 62%, 63%, 64%, 65%, 66%, 67%, 68%, 69%, 70%, 75%, 80%, 85%, 90%, 95%, or 100%)). In some embodiments, the treatment results in an ORR of the population of subjects of at least about 53% (e.g., about 65% to about 100% (e.g., 65%, 65.5%, 66%, 66.5%, 67%, 67.5%, 68%, 68.5%, 69%, 69.5%, 70%, 71%, 72%, 73%, 74%, 75%, 76%, 77%, 78%, 79%, 80%, 85%, 90%, 95%, or 100%)). In some embodiments, the treatment results in an ORR of the population of subjects of at least about 53% to at least about 67.5% (e.g., at least about 54%, 55%, 56%, 57%, 58%, 59%, 60%, 61%, 62%, 63%, 64%, 65%, 66%, 67%, or 67.5%).

E. Therapeutic Methods and Diagnostic and Uses Relating to Head and Neck Cancers

Head and Neck Cancers

Head and neck cancers are a cause of significant morbidity and mortality, accounting for 890,000 new cases and 450,000 deaths globally in 2018. Head and neck cancers are a heterogeneous group, comprising of cancers that begin in the mucosal surfaces of the upper aerodigestive tract and affect the oral cavity, oropharynx, larynx, hypopharynx, and nasopharynx. The dominant histological type is squamous cell carcinoma (SCC), and accounts for over 90% of all malignant disease in the head and neck region of the body.

Despite advances in diagnosis and treatment of early stage or locally advanced squamous cell carcinoma of the head and neck (SCCHN), more than 65% of these patients will develop recurrent or metastatic disease. In addition, approximately 10% of SCCHN patients will present with metastatic SCCHN at initial diagnosis. For patients with locally recurrent disease, salvage surgery is curative only for select patients with resectable locoregional recurrence, and re-irradiation is often limited by prior radiotherapy history and associated toxicity and morbidity. As a result, for patients with recurrent or metastatic SCCHN, systemic therapy is a standard-of-care (SOC) therapy and mainstay of palliation. For these patients, the prognosis is poor with a median survival of 6-15 months in most clinical trials, depending upon patient and disease related-factors.

Therefore, there is a high unmet need for improved medical intervention of head and neck cancers, and, in particular, SCCHN.

Methods and Uses for Treating Head and Neck Cancers

Provided herein are methods and uses for treating an SCCHN (e.g., a recurrent and/or metastatic SCCHN (e.g., a PD-L1-positive and/or HPV-positive recurrent and/or metastatic SCCHN)) in a subject or population of subjects comprising administering to the subject or population of subjects a dosing regimen comprising one or more dosing cycles of an effective amount of a PD-1 axis binding antagonist (e.g., an anti-PD-L1 antagonist antibody (e.g., atezolizumab) or an anti-PD-1 antagonist antibody (e.g., MDX 1106 (nivolumab), MK-3475 (pembrolizumab, previously known as lambrolizumab), MEDI-0680 (AMP-514), PDR001 (spartalizumab), REGN2810 (cemiplimab), BGB-108, prolgolimab, camrelizumab, sintilimab, tislelizumab, or toripalimab)) and an anti-TIGIT antagonist antibody (e.g., an anti-TIGIT antagonist antibody as disclosed herein, e.g., tiragolumab), thereby treating the subject or population of subjects. Also provided herein are methods for treating a subject having an SCCHN with a detectable expression level of PD-L1 comprising administering to the subject one or more dosing cycles of an anti-TIGIT antagonist antibody at a dose of between about 30 mg to about 1200 mg every three weeks and a PD-1 axis binding antagonist at a dose of between about 80 mg to about 1600 mg every three weeks. Also provided herein are methods for treating a subject having an SCCHN with a detectable expression level of PD-L1 comprising administering to the subject one or more dosing cycles of tiragolumab at a dose of about 600 mg every three weeks and atezolizumab at a dose of about 1200 mg every three weeks.

The present invention includes methods and uses involving administration of an effective amount of a PD-1 axis binding antagonist (e.g., an anti-PD-L1 antagonist antibody, such as atezolizumab) and an anti-TIGIT antagonist antibody (e.g., an anti-TIGIT antagonist antibody as disclosed herein, e.g., tiragolumab) to a subject or population of subjects in need thereof. In some instances, the invention includes methods and uses involving administration of an effective amount of a PD-1 axis binding antagonist (e.g., an anti-PD-L1 antagonist antibody, such as atezolizumab) and an anti-TIGIT antagonist antibody (e.g., an anti-TIGIT antagonist antibody as disclosed herein, e.g., tiragolumab) to a subject or population of subjects in need thereof.

In some instances, any of the methods and uses for treating an SCCHN in a subject or population of subjects can result in CR or PR. In some instances, any of the methods and uses for treating an SCCHN in a subject or population of subjects can result in an increase in the objective response rate (ORR) of the subject or population of subjects. In some instances, any of the methods and uses for treating an SCCHN in a subject or population of subjects can result in an increase in the PFS, duration of response (DOR), and/or OS of the subject or population of subjects.

In some instances, the SCCHN is an SCCHN with a detectable expression level of PD-L1. In some instances, a tumor sample obtained from the subject or population of subjects has been determined to have a detectable expression level of PD-L1 (e.g., a detectable protein and/or nucleic acid expression level of PD-L1 (e.g., a tumor-associated immune-cell (TIC) of greater than or equal to 5%)). In some instances, the detectable protein expression level of PD-L1 is a TIC of greater than or equal to 5% (e.g., PD-L1-positive), greater than or equal to 5% and less than 20% (e.g., PD-L1 low), or greater than or equal to 20% (e.g., PD-L1 high) in the tumor sample. In some instances, the detectable protein expression level of PD-L1 is a TIC of greater than or equal to 10%, greater than or equal to 10% and less than 50%, or greater than or equal to 50% in the tumor sample.

In some instances, the treatment is a first-line treatment. In some instances, the subject or population of subjects has not received prior therapy. In some instances, the subject or population of subjects has not received a prior systemic therapy for recurrent and/or metastatic disease.

Dosing Regimens and Administration

The therapeutic methods and uses of the invention described herein include, in one aspect, administering to a subject or population of subjects having an SCCHN (e.g., a recurrent and/or metastatic SCCHN) a dosing regimen comprising one or more dosing cycles of an effective amount of a PD-1 axis binding antagonist (e.g., an anti-PD-L1 antagonist antibody (e.g., atezolizumab) or an anti-PD-1 antagonist antibody (e.g., MDX-1106 (nivolumab) or MK-3475 (pembrolizumab, previously known as lambrolizumab))) and one or more dosing cycles of an effective amount of an anti-TIGIT antagonist antibody (e.g., an anti-TIGIT antagonist antibody as disclosed herein, e.g., tiragolumab).

The pharmaceutical compositions described herein can be formulated for administration as described in Section III(K).

Diagnostic Methods and Uses for Head and Neck Cancer

The invention provides methods for selecting a therapy (e.g., a first-line therapy) for a subject or population of subjects having an SCCHN (e.g., a recurrent and/or metastatic SCCHN), wherein therapy is guided by diagnostic methods that involve determining the presence and/or expression levels/amount of one or more biomarkers (e.g., PD-L1 (e.g., as determined by PD-L1 protein and/or nucleic acid expression) or HPV status (e.g., as determined by p16 IHC, ISH, or PCR)) in a sample (e.g., a tumor sample or a blood sample) obtained from the subject or population of subjects.

Additionally provided herein are methods for identifying a subject or population of subjects having an SCCHN (e.g., a recurrent and/or metastatic SCCHN) who may benefit from a treatment comprising an anti-TIGIT antagonist antibody and a PD-1 axis binding antagonist (e.g., an anti-PD-L1 antagonist antibody (e.g., atezolizumab) or an anti-PD-1 antagonist antibody (e.g., MDX 1106 (nivolumab), MK-3475 (pembrolizumab, previously known as lambrolizumab), MEDI-0680 (AMP-514), PDR001 (spartalizumab), REGN2810 (cemiplimab), BGB-108, prolgolimab, camrelizumab, sintilimab, tislelizumab, or toripalimab)), wherein identification is guided by diagnostic methods that involve determining the presence and/or expression levels/amount of one or more biomarkers (e.g., PD-L1 (e.g., as determined by PD-L1 protein and/or nucleic acid expression) or HPV status (e.g., as determined by p16 IHC, ISH, or PCR)) in a sample (e.g., a tumor sample or a blood sample) obtained from the subject or population of subjects.

Additionally provided herein are methods for assessing responsiveness to a therapy for a subject or population of subjects having an SCCHN (e.g., a recurrent and/or metastatic SCCHN), wherein further therapy is guided by diagnostic methods that involve determining the presence and/or expression levels/amount of one or more biomarkers (e.g., PD-L1 (e.g., as determined by PD-L1 protein and/or nucleic acid expression) or HPV status (e.g., as determined by p16 IHC, ISH, or PCR)) in a sample (e.g., a tumor sample or a blood sample) obtained from the subject or population of subjects.

Additionally provided herein are methods for optimizing a therapy for a subject or population of subjects having an SCCHN (e.g., a recurrent and/or metastatic SCCHN), wherein further therapy is guided by diagnostic methods that involve determining the presence and/or expression levels/amount of one or more biomarkers (e.g., PD-L1 (e.g., as determined by PD-L1 protein and/or nucleic acid expression) or HPV status (e.g., as determined by p16 IHC, ISH, or PCR)) in a sample (e.g., a tumor sample or a blood sample) obtained from the subject or population of subjects.

Biomarkers for use in the methods described herein can include, but are not limited to, PD-L1 expression on tissues (e.g., tumor tissues) or in blood (e.g., whole blood), germline and somatic mutations from tissue (e.g., tumor tissue) and/or from circulating tumor DNA in blood (including, but not limited to, mutation load, MSI, and MMR defects), identified through WGS and/or NGS, analysis of genes or gene signatures associated with tumor immunobiology, HPV alterations, lymphocyte subpopulations, T cell-receptor repertoire, cytokines associated with T-cell activation, and plasma derived cytokines. In some instances, the biomarker is PD-L1. In some instances, a tumor sample obtained from the subject or population of subjects has been determined to have a detectable expression level of PD-L1 (e.g., a detectable protein and/or nucleic acid expression level of PD-L1 (e.g., a tumor-associated immune-cell (TIC) of greater than or equal to 5%)). In some instances, the detectable protein expression level of PD-L1 is a tumor-associated immune-cell (TIC) of greater than or equal to 5% (e.g., PD-L1-positive), greater than or equal to 5% and less than 20% (e.g., PD-L1 low), or greater than or equal to 20% (e.g., PD-L1 high) in the tumor sample. In some instances, the detectable protein expression level of PD-L1 is a TIC of greater than or equal to 10%, greater than or equal to 10% and less than 50%, or greater than or equal to 50% in the tumor sample. In some instances, the sample is a tumor sample (e.g., a formalin-fixed, paraffin-embedded (FFPE) tumor sample). In some instances, the tumor sample is a tumor tissue sample.

In some instances, the method includes determining the presence and/or expression levels/amount of a (e.g., PD-L1 (e.g., as determined by PD-L1 protein and/or nucleic acid expression) or HPV status (e.g., as determined by p16 IHC, ISH, or PCR)) in a sample (e.g., a tumor sample or a blood sample) from the subject or population of subjects, and administering to the subject or population of subjects one or more dosing cycles of an anti-TIGIT antagonist antibody (e.g., an anti-TIGIT antagonist antibody disclosed herein, e.g., tiragolumab) at a dose (e.g., a fixed dose) of between about 30 mg to about 1200 mg (e.g., 30 mg to 1200 mg) every three weeks and one or more dosing cycles of a PD-1 axis binding antagonist (e.g., an anti-PD-L1 antagonist antibody (e.g., atezolizumab)) at a dose (e.g., a fixed dose) of between about 80 mg to about 2000 mg (e.g., 80 mg to 2000 mg) every three weeks. In some instances, the method includes determining the presence and/or expression levels/amount of a (e.g., PD-L1 (e.g., as determined by PD-L1 protein and/or nucleic acid expression) or HPV status (e.g., as determined by p16 IHC, ISH, or PCR)) in a sample (e.g., a tumor sample or a blood sample) from the subject or population of subjects, and administering to the subject or population of subjects one or more dosing cycles of an anti-TIGIT antagonist antibody (e.g., an anti-TIGIT antagonist antibody disclosed herein, e.g., tiragolumab) at a dose of about 600 mg (e.g., 600 mg) every three weeks and one or more dosing cycles of a PD-1 axis binding antagonist (e.g., an anti-PD-L1 antagonist antibody (e.g., atezolizumab)) at a dose of 840 mg every two weeks. In some instances, the method includes determining the presence and/or expression levels/amount of a (e.g., PD-L1 (e.g., as determined by PD-L1 protein and/or nucleic acid expression) or HPV status (e.g., as determined by p16 IHC, ISH, or PCR)) in a sample (e.g., a tumor sample or a blood sample) from the subject or population of subjects, and administering to the subject or population of subjects one or more dosing cycles of an anti-TIGIT antagonist antibody (e.g., an anti-TIGIT antagonist antibody disclosed herein, e.g., tiragolumab) at a dose of about 600 mg (e.g., 600 mg) every three weeks and one or more dosing cycles of a PD-1 axis binding antagonist (e.g., an anti-PD-L1 antagonist antibody (e.g., atezolizumab)) at a dose of 1200 mg every three weeks. In some instances, the method includes determining the presence and/or expression levels/amount of a (e.g., PD-L1 (e.g., as determined by PD-L1 protein and/or nucleic acid expression) or HPV status (e.g., as determined by p16 IHC, ISH, or PCR)) in a sample (e.g., a tumor sample or a blood sample) from the subject or population of subjects, and administering to the subject or population of subjects one or more dosing cycles of an anti-TIGIT antagonist antibody (e.g., an anti-TIGIT antagonist antibody disclosed herein, e.g., tiragolumab) at a dose of about 600 mg (e.g., 600 mg) every three weeks and one or more dosing cycles of a PD-1 axis binding antagonist (e.g., an anti-PD-L1 antagonist antibody (e.g., atezolizumab)) at a dose of 1680 mg every four weeks.

Presence and/or expression levels/amount of a (e.g., PD-L1 (e.g., as determined by PD-L1 protein and/or nucleic acid expression) or HPV status (e.g., as determined by p16 IHC, ISH, or PCR)) can be determined qualitatively and/or quantitatively based on any suitable criterion known in the art, including but not limited to proteins, protein fragments, DNA, mRNA, cDNA, and/or gene copy number.

In some instances, expression levels or amount of a biomarker is a detectable protein expression level of PD-L1 in a tumor sample (e.g., a FFPE tumor sample) from the subject or population of subjects. In some instances, the PD-L1 protein expression level has been determined by an IHC assay. In some instances, the tumor sample is an FFPE tumor sample.

In some instances, the tumor sample (e.g., FFPE tumor sample) from the subject or population of subjects has been determined to have a detectable expression level of PD-L1. In some instances, the tumor sample (e.g., FFPE tumor sample) from the subject or population of subjects has been determined to have a detectable expression level of PD-L1 in tumor-infiltrating immune cells. In some instances, the tumor sample is an FFPE tumor sample.

In some instances, the expression levels or amount of a biomarker is a detectable nucleic acid expression level of PD-L1 in a tumor sample (e.g., FFPE tumor sample) from the subject or population of subjects. In some instances, the PD-L1 nucleic acid expression level has been determined by RNA-seq, RT-qPCR, qPCR, multiplex qPCR, or RT-qPCR, microarray analysis, serial analysis of gene expression (SAGE), MassARRAY® technique, in situ hybridization (ISH), or a combination thereof. In some instances, the tumor sample is an FFPE tumor sample.

In some instances, the presence and/or expression levels/amount of the (e.g., PD-L1 (e.g., as determined by PD-L1 protein and/or nucleic acid expression) or HPV status (e.g., as determined by p16 IHC, ISH, or PCR)) in a sample (e.g., a tumor sample or a blood sample) from a subject or population of subjects selects the subject or population of subjects as eligible for therapy with an anti-TIGIT antagonist antibody and a PD-1 axis binding antagonist (e.g., an anti-PD-L1 antagonist antibody (e.g., atezolizumab) or an anti-PD-1 antagonist antibody (e.g., MDX-1106 (nivolumab) or MK-3475 (pembrolizumab, previously known as lambrolizumab))), for example, where a detectable expression level of PD-L1 is a biomarker for selection of individuals. In some instances, the sample is selected from the group consisting of a tissue sample, a whole blood sample, a serum sample, and a plasma sample. In some instances, the tissue sample is a tumor sample (e.g., FFPE tumor sample). In some instances, the tumor sample comprises tumor-infiltrating immune cells, tumor cells, stromal cells, and any combinations thereof. In some instances, the tumor sample is an FFPE tumor sample.

In one aspect, the invention provides methods for selecting a therapy for a subject or population of subjects having an SCCHN (e.g., a recurrent and/or metastatic SCCHN) by obtaining a tumor sample (e.g., a biopsy) from the subject or population of subjects, detecting the protein expression level of PD-L1 in the tumor sample by an IHC assay using an anti-PD-L1 antibody suitable for staining, and identifying the subject or population of subjects as one who is likely to benefit from a therapy comprising one or more dosing cycles of an anti-TIGIT antagonist antibody administered at a dose (e.g., a fixed dose) of 600 mg every three weeks and atezolizumab administered at a dose (e.g., a fixed dose) of 1200 mg every three weeks based on PD-L1 expression in the tumor sample having been detected. In some instances, the therapy comprises one or more dosing cycles of an anti-TIGIT antagonist antibody administered at a dose of 600 mg every three weeks and atezolizumab administered at a dose of 1680 mg every four weeks. In some instances, the therapy comprises one or more dosing cycles of an anti-TIGIT antagonist antibody administered at a dose of 600 mg every three weeks and atezolizumab administered at a dose of 840 mg every two weeks. In some instances, the method further includes administering to the identified subject or population of subjects the therapy. In another aspect, the invention provides methods for selecting a therapy for a subject or population of subjects having an SCCHN (e.g., a recurrent and/or metastatic SCCHN) by obtaining a tumor sample (e.g., a biopsy) from the subject or population of subjects, detecting the protein expression level of PD-L1 in the tumor sample by an IHC assay using an anti-PD-L1 antibody suitable for staining, and identifying the subject or population of subjects as one who is likely to benefit from a therapy comprising one or more dosing cycles of tiragolumab administered at a dose of 600 mg every three weeks and atezolizumab administered at a dose of 1200 mg every three weeks based on PD-L1 expression in the tumor sample having been detected. In some instances, the therapy may further include, or be administered in conjunction with (either separately or together), one or more additional anti-cancer therapeutic agent(s) (e.g., an immunomodulatory agent (e.g., an agent that decreases or inhibits one or more immune co-inhibitory receptors (e.g., one or more immune co-inhibitory receptors selected from TIGIT, PD-L1, PD-1, CTLA-4, LAG3, TIM3, BTLA, and/or VISTA), such as a CTLA-4 antagonist, e.g., an anti-CTLA-4 antagonist antibody (e.g., ipilimumab (YERVOY®)), or an agent that increases or activates one or more immune co-stimulatory receptors (e.g., one or more immune co-stimulatory receptors selected from CD226, OX-40, CD28, CD27, CD137, HVEM, and/or GITR), such as an OX-40 agonist, e.g., an OX-40 agonist antibody), a chemotherapeutic agent, a cytotoxic agent, a growth inhibitory agent, a radiotherapy/radiation therapy, and/or an anti-hormonal agent, such as those recited herein above).

In another aspect, the invention provides methods for selecting a therapy for a subject or population of subjects having an SCCHN (e.g., a recurrent and/or metastatic SCCHN) by obtaining a tumor sample (e.g., a biopsy) from the subject or population of subjects, detecting the protein expression level of PD-L1 in the tumor sample by an IHC assay using an anti-PD-L1 antibody suitable for staining, and identifying the subject or population of subjects as one who is likely to benefit from a therapy comprising one or more dosing cycles of an anti-TIGIT antagonist antibody administered at a dose (e.g., a fixed dose) of 600 mg every three weeks and atezolizumab administered at a dose (e.g., a fixed dose) of 1200 mg every three weeks based on PD-L1 expression in the tumor sample having been detected. In some instances, the therapy comprises one or more dosing cycles of an anti-TIGIT antagonist antibody administered at a dose of 600 mg every three weeks and atezolizumab administered at a dose of 1680 mg every four weeks. In some instances, the therapy comprises one or more dosing cycles of an anti-TIGIT antagonist antibody administered at a dose of 600 mg every three weeks and atezolizumab administered at a dose of 840 mg every two weeks. In another aspect, the invention provides methods for selecting a therapy for a subject or population of subjects having an SCCHN (e.g., a recurrent and/or metastatic SCCHN) by obtaining a tumor sample (e.g., a biopsy) from the subject or population of subjects, detecting the protein expression level of PD-L1 in the tumor sample by an IHC assay using an anti-PD-L1 antibody suitable for staining, and identifying the subject or population of subjects as one who is likely to benefit from a therapy comprising one or more dosing cycles of tiragolumab administered at a dose of 600 mg every three weeks and atezolizumab administered at a dose of 1200 mg every three weeks based on PD-L1 expression in the tumor sample having been detected. In some instances, the method further includes administering to the identified subject or population of subjects the therapy. In some instances, the therapy may further include, or be administered in conjunction with (either separately or together), one or more additional anti-cancer therapeutic agent(s) (e.g., an immunomodulatory agent (e.g., an agent that decreases or inhibits one or more immune co-inhibitory receptors (e.g., one or more immune co-inhibitory receptors selected from TIGIT, PD-L1, PD-1, CTLA-4, LAG3, TIM3, BTLA, and/or VISTA), such as a CTLA-4 antagonist, e.g., an anti-CTLA-4 antagonist antibody (e.g., ipilimumab (YERVOY®)), or an agent that increases or activates one or more immune co-stimulatory receptors (e.g., one or more immune co-stimulatory receptors selected from CD226, OX-40, CD28, CD27, CD137, HVEM, and/or GITR), such as an OX-40 agonist, e.g., an OX-40 agonist antibody), a chemotherapeutic agent, a cytotoxic agent, a growth inhibitory agent, a radiotherapy/radiation therapy, and/or an anti-hormonal agent, such as those recited herein above).

In another aspect, the invention provides methods for selecting a therapy for a subject or population of subjects having an SCCHN (e.g., a recurrent and/or metastatic SCCHN) by obtaining a tumor sample (e.g., a biopsy) from the subject or population of subjects, detecting the protein expression level of PD-L1 in the tumor sample by an IHC assay using an anti-PD-L1 antibody suitable for staining, and identifying the subject or population of subjects as one who is likely to benefit from a therapy comprising one or more dosing cycles of an anti-TIGIT antagonist antibody administered at a dose (e.g., a fixed dose) of 600 mg every three weeks and atezolizumab administered at a dose (e.g., a fixed dose) of 1200 mg every three weeks based on PD-L1 expression in the tumor sample having been detected. In some instances, the therapy comprises one or more dosing cycles of an anti-TIGIT antagonist antibody administered at a dose of 600 mg every three weeks and atezolizumab administered at a dose of 1680 mg every four weeks. In some instances, the therapy comprises one or more dosing cycles of an anti-TIGIT antagonist antibody administered at a dose of 600 mg every three weeks and atezolizumab administered at a dose of 840 mg every two weeks. In another aspect, the invention provides methods for selecting a therapy for a subject or population of subjects having an SCCHN (e.g., a recurrent and/or metastatic SCCHN) by obtaining a tumor sample (e.g., a biopsy) from the subject or population of subjects, detecting the protein expression level of PD-L1 in the tumor sample by an IHC assay using an anti-PD-L1 antibody suitable for staining, and identifying the subject or population of subjects as one who is likely to benefit from a therapy comprising one or more dosing cycles of tiragolumab administered at a dose of 600 mg every three weeks and atezolizumab administered at a dose of 1200 mg every three weeks based on PD-L1 expression in the tumor sample having been detected. In some instances, the method further includes administering to the identified subject or population of subjects the therapy. In some instances, the therapy may further include, or be administered in conjunction with (either separately or together), one or more additional anti-cancer therapeutic agent(s) (e.g., an immunomodulatory agent (e.g., an agent that decreases or inhibits one or more immune co-inhibitory receptors (e.g., one or more immune co-inhibitory receptors selected from TIGIT, PD-L1, PD-1, CTLA-4, LAG3, TIM3, BTLA, and/or VISTA), such as a CTLA-4 antagonist, e.g., an anti-CTLA-4 antagonist antibody (e.g., ipilimumab (YERVOY®)), or an agent that increases or activates one or more immune co-stimulatory receptors (e.g., one or more immune co-stimulatory receptors selected from CD226, OX-40, CD28, CD27, CD137, HVEM, and/or GITR), such as an OX-40 agonist, e.g., an OX-40 agonist antibody), a chemotherapeutic agent, a cytotoxic agent, a growth inhibitory agent, a radiotherapy/radiation therapy, and/or an anti-hormonal agent, such as those recited herein above).

In some instances, the invention provides methods for identifying a subject or population of subjects having an SCCHN (e.g., a recurrent and/or metastatic SCCHN) who may benefit from a therapy comprising an anti-TIGIT antagonist antibody and a PD-1 axis binding antagonist (e.g., an anti-PD-L1 antagonist antibody (e.g., atezolizumab) or an anti-PD-1 antagonist antibody (e.g., MDX-1106 (nivolumab) or MK-3475 (pembrolizumab, previously known as lambrolizumab))), by obtaining a tumor sample (e.g., a biopsy) from the subject or population of subjects, detecting the protein expression level of PD-L1 in the tumor sample by an IHC assay using an anti-PD-L1 antibody suitable for staining, and identifying the subject or population of subjects as one who is likely to benefit from a therapy comprising one or more dosing cycles of an anti-TIGIT antagonist antibody administered at a dose (e.g., a fixed dose) of 600 mg every three weeks and atezolizumab administered at a dose (e.g., a fixed dose) of 1200 mg every three weeks. In some instances, the therapy comprises one or more dosing cycles of an anti-TIGIT antagonist antibody administered at a dose of 600 mg every three weeks and atezolizumab administered at a dose of 1680 mg every four weeks. In some instances, the therapy comprises one or more dosing cycles of an anti-TIGIT antagonist antibody administered at a dose of 600 mg every three weeks and atezolizumab administered at a dose of 840 mg every two weeks. In some instances, the invention provides methods for identifying a subject or population of subjects having an SCCHN (e.g., a recurrent and/or metastatic SCCHN) who may benefit from a therapy comprising an anti-TIGIT antagonist antibody and a PD-1 axis binding antagonist (e.g., an anti-PD-L1 antagonist antibody (e.g., atezolizumab) or an anti-PD-1 antagonist antibody (e.g., MDX-1106 (nivolumab) or MK-3475 (pembrolizumab, previously known as lambrolizumab))), by obtaining a tumor sample (e.g., a biopsy) from the subject or population of subjects, detecting the protein expression level of PD-L1 in the tumor sample by an IHC assay using an anti-PD-L1 antibody suitable for staining, and identifying the subject or population of subjects as one who is likely to benefit from a therapy comprising one or more dosing cycles of tiragolumab administered at a dose of 600 mg every three weeks and atezolizumab administered at a dose of 1200 mg every three weeks. In some instances, the method further includes administering to the identified subject or population of subjects the therapy. In some instances, the therapy may further include, or be administered in conjunction with (either separately or together), one or more additional anti-cancer therapeutic agent(s) (e.g., an immunomodulatory agent (e.g., an agent that decreases or inhibits one or more immune co-inhibitory receptors (e.g., one or more immune co-inhibitory receptors selected from TIGIT, PD-L1, PD-1, CTLA-4, LAG3, TIM3, BTLA, and/or VISTA), such as a CTLA-4 antagonist, e.g., an anti-CTLA-4 antagonist antibody (e.g., ipilimumab (YERVOY®)), or an agent that increases or activates one or more immune co-stimulatory receptors (e.g., one or more immune co-stimulatory receptors selected from CD226, OX-40, CD28, CD27, CD137, HVEM, and/or GITR), such as an OX-40 agonist, e.g., an OX-40 agonist antibody), a chemotherapeutic agent, a cytotoxic agent, a growth inhibitory agent, a radiotherapy/radiation therapy, and/or an anti-hormonal agent, such as those recited herein above).

In some instances, the invention provides methods for identifying a subject or population of subjects having an SCCHN (e.g., a recurrent and/or metastatic SCCHN) who may benefit from a therapy comprising an anti-TIGIT antagonist antibody and a PD-1 axis binding antagonist (e.g., an anti-PD-L1 antagonist antibody (e.g., atezolizumab) or an anti-PD-1 antagonist antibody (e.g., MDX-1106 (nivolumab) or MK-3475 (pembrolizumab, previously known as lambrolizumab))), by obtaining a tumor sample (e.g., a biopsy) from the subject or population of subjects, detecting the protein expression level of PD-L1 in the tumor sample by an IHC assay using an anti-PD-L1 antibody suitable for staining, and identifying the subject or population of subjects as one who is likely to benefit from a therapy comprising one or more dosing cycles of an anti-TIGIT antagonist antibody administered at a dose (e.g., a fixed dose) of 600 mg every three weeks and atezolizumab administered at a dose (e.g., a fixed dose) of 1200 mg every three weeks. In some instances, the therapy comprises one or more dosing cycles of an anti-TIGIT antagonist antibody administered at a dose of 600 mg every three weeks and atezolizumab administered at a dose of 1680 mg every four weeks. In some instances, the therapy comprises one or more dosing cycles of an anti-TIGIT antagonist antibody administered at a dose of 600 mg every three weeks and atezolizumab administered at a dose of 840 mg every two weeks. In some instances, the invention provides methods for identifying a subject or population of subjects having an SCCHN (e.g., a recurrent and/or metastatic SCCHN) who may benefit from a therapy comprising an anti-TIGIT antagonist antibody and a PD-1 axis binding antagonist (e.g., an anti-PD-L1 antagonist antibody (e.g., atezolizumab) or an anti-PD-1 antagonist antibody (e.g., MDX-1106 (nivolumab) or MK-3475 (pembrolizumab, previously known as lambrolizumab))), by obtaining a tumor sample (e.g., a biopsy) from the subject or population of subjects, detecting the protein expression level of PD-L1 in the tumor sample by an IHC assay using an anti-PD-L1 antibody suitable for staining, and identifying the subject or population of subjects as one who is likely to benefit from a therapy comprising one or more dosing cycles of tiragolumab administered at a dose (e.g., a fixed dose) of 600 mg every three weeks and atezolizumab administered at a dose (e.g., a fixed dose) of 1200 mg every three weeks. In some instances, the method further includes administering to the identified subject or population of subjects the therapy. In some instances, the therapy may further include, or be administered in conjunction with (either separately or together), one or more additional anti-cancer therapeutic agent(s) (e.g., an immunomodulatory agent (e.g., an agent that decreases or inhibits one or more immune co-inhibitory receptors (e.g., one or more immune co-inhibitory receptors selected from TIGIT, PD-L1, PD-1, CTLA-4, LAG3, TIM3, BTLA, and/or VISTA), such as a CTLA-4 antagonist, e.g., an anti-CTLA-4 antagonist antibody (e.g., ipilimumab (YERVOY®)), or an agent that increases or activates one or more immune co-stimulatory receptors (e.g., one or more immune co-stimulatory receptors selected from CD226, OX-40, CD28, CD27, CD137, HVEM, and/or GITR), such as an OX-40 agonist, e.g., an OX-40 agonist antibody), a chemotherapeutic agent, a cytotoxic agent, a growth inhibitory agent, a radiotherapy/radiation therapy, and/or an anti-hormonal agent, such as those recited herein above).

In some instances, the invention provides methods for identifying a subject or population of subjects having an SCCHN (e.g., a recurrent and/or metastatic SCCHN) who may benefit from a therapy comprising an anti-TIGIT antagonist antibody and a PD-1 axis binding antagonist (e.g., an anti-PD-L1 antagonist antibody (e.g., atezolizumab) or an anti-PD-1 antagonist antibody (e.g., MDX-1106 (nivolumab) or MK-3475 (pembrolizumab, previously known as lambrolizumab))), by obtaining a tumor sample (e.g., a biopsy) from the subject or population of subjects, detecting the protein expression level of PD-L1 in the tumor sample by an IHC assay using an anti-PD-L1 antibody suitable for staining, and identifying the subject or population of subjects as one who is likely to benefit from a therapy comprising one or more dosing cycles of an anti-TIGIT antagonist antibody administered at a dose (e.g., a fixed dose) of 600 mg every three weeks and atezolizumab administered at a dose (e.g., a fixed dose) of 1200 mg every three weeks. In some instances, the therapy comprises one or more dosing cycles of an anti-TIGIT antagonist antibody administered at a dose of 600 mg every three weeks and atezolizumab administered at a dose of 1680 mg every four weeks. In some instances, the therapy comprises one or more dosing cycles of an anti-TIGIT antagonist antibody administered at a dose of 600 mg every three weeks and atezolizumab administered at a dose of 840 mg every two weeks. In some instances, the invention provides methods for identifying a subject or population of subjects having an SCCHN (e.g., a recurrent and/or metastatic SCCHN) who may benefit from a therapy comprising an anti-TIGIT antagonist antibody and a PD-1 axis binding antagonist (e.g., an anti-PD-L1 antagonist antibody (e.g., atezolizumab) or an anti-PD-1 antagonist antibody (e.g., MDX-1106 (nivolumab) or MK-3475 (pembrolizumab, previously known as lambrolizumab))), by obtaining a tumor sample (e.g., a biopsy) from the subject or population of subjects, detecting the protein expression level of PD-L1 in the tumor sample by an IHC assay using an anti-PD-L1 antibody suitable for staining, and identifying the subject or population of subjects as one who is likely to benefit from a therapy comprising one or more dosing cycles of tiragolumab administered at a dose of 600 mg every three weeks and atezolizumab administered at a dose of 1200 mg every three weeks. In some instances, the method further includes administering to the identified subject or population of subjects the therapy. In some instances, the therapy may further include, or be administered in conjunction with (either separately or together), one or more additional anti-cancer therapeutic agent(s) (e.g., an immunomodulatory agent (e.g., an agent that decreases or inhibits one or more immune co-inhibitory receptors (e.g., one or more immune co-inhibitory receptors selected from TIGIT, PD-L1, PD-1, CTLA-4, LAG3, TIM3, BTLA, and/or VISTA), such as a CTLA-4 antagonist, e.g., an anti-CTLA-4 antagonist antibody (e.g., ipilimumab (YERVOY®)), or an agent that increases or activates one or more immune co-stimulatory receptors (e.g., one or more immune co-stimulatory receptors selected from CD226, OX-40, CD28, CD27, CD137, HVEM, and/or GITR), such as an OX-40 agonist, e.g., an OX-40 agonist antibody), a chemotherapeutic agent, a cytotoxic agent, a growth inhibitory agent, a radiotherapy/radiation therapy, and/or an anti-hormonal agent, such as those recited herein above).

In some instances, the invention provides methods for assessing responsiveness of a subject or population of subjects having an SCCHN (e.g., a recurrent and/or metastatic SCCHN) to a therapy comprising an anti-TIGIT antagonist antibody and a PD-1 axis binding antagonist (e.g., an anti-PD-L1 antagonist antibody (e.g., atezolizumab) or an anti-PD-1 antagonist antibody (e.g., MDX-1106 (nivolumab) or MK-3475 (pembrolizumab, previously known as lambrolizumab))), by obtaining a tumor sample (e.g., a biopsy) from the subject or population of subjects, detecting the protein expression level of PD-L1 in the tumor sample by an IHC assay using an anti-PD-L1 antibody suitable for staining, and identifying the subject or population of subjects as one who is likely to benefit from a therapy comprising one or more dosing cycles of an anti-TIGIT antagonist antibody administered at a dose (e.g., a fixed dose) of 600 mg every three weeks and atezolizumab administered at a dose (e.g., a fixed dose) of 1200 mg every three weeks. In some instances, the therapy comprises one or more dosing cycles of an anti-TIGIT antagonist antibody administered at a dose of 600 mg every three weeks and atezolizumab administered at a dose of 1680 mg every four weeks. In some instances, the therapy comprises one or more dosing cycles of an anti-TIGIT antagonist antibody administered at a dose of 600 mg every three weeks and atezolizumab administered at a dose of 840 mg every two weeks. In some instances, the invention provides methods for assessing responsiveness of a subject or population of subjects having an SCCHN (e.g., a recurrent and/or metastatic SCCHN) to a therapy comprising an anti-TIGIT antagonist antibody and a PD-1 axis binding antagonist (e.g., an anti-PD-L1 antagonist antibody (e.g., atezolizumab) or an anti-PD-1 antagonist antibody (e.g., MDX-1106 (nivolumab) or MK-3475 (pembrolizumab, previously known as lambrolizumab))), by obtaining a tumor sample (e.g., a biopsy) from the subject or population of subjects, detecting the protein expression level of PD-L1 in the tumor sample by an IHC assay using an anti-PD-L1 antibody suitable for staining, and identifying the subject or population of subjects as one who is likely to benefit from a therapy comprising one or more dosing cycles of tiragolumab administered at a dose of 600 mg every three weeks and atezolizumab administered at a dose of 1200 mg every three weeks. In some instances, the method further includes administering to the identified subject or population of subjects the therapy. In some instances, the therapy may further include, or be administered in conjunction with (either separately or together), one or more additional anti-cancer therapeutic agent(s) (e.g., an immunomodulatory agent (e.g., an agent that decreases or inhibits one or more immune co-inhibitory receptors (e.g., one or more immune co-inhibitory receptors selected from TIGIT, PD-L1, PD-1, CTLA-4, LAG3, TIM3, BTLA, and/or VISTA), such as a CTLA-4 antagonist, e.g., an anti-CTLA-4 antagonist antibody (e.g., ipilimumab (YERVOY®)), or an agent that increases or activates one or more immune co-stimulatory receptors (e.g., one or more immune co-stimulatory receptors selected from CD226, OX-40, CD28, CD27, CD137, HVEM, and/or GITR), such as an OX-40 agonist, e.g., an OX-40 agonist antibody), a chemotherapeutic agent, a cytotoxic agent, a growth inhibitory agent, a radiotherapy/radiation therapy, and/or an anti-hormonal agent, such as those recited herein above).

In some instances, the invention provides methods for assessing responsiveness of a subject or population of subjects having an SCCHN (e.g., a recurrent and/or metastatic SCCHN) to a therapy comprising an anti-TIGIT antagonist antibody and a PD-1 axis binding antagonist (e.g., an anti-PD-L1 antagonist antibody (e.g., atezolizumab) or an anti-PD-1 antagonist antibody (e.g., MDX-1106 (nivolumab) or MK-3475 (pembrolizumab, previously known as lambrolizumab))), by obtaining a tumor sample (e.g., a biopsy) from the subject or population of subjects, detecting the protein expression level of PD-L1 in the tumor sample by an IHC assay using an anti-PD-L1 antibody suitable for staining, and identifying the subject or population of subjects as one who is likely to benefit from a therapy comprising one or more dosing cycles of an anti-TIGIT antagonist antibody administered at a dose (e.g., a fixed dose) of 600 mg every three weeks and atezolizumab administered at a dose (e.g., a fixed dose) of 1200 mg every three weeks. In some instances, the therapy comprises one or more dosing cycles of an anti-TIGIT antagonist antibody administered at a dose of 600 mg every three weeks and atezolizumab administered at a dose of 1680 mg every four weeks. In some instances, the therapy comprises one or more dosing cycles of an anti-TIGIT antagonist antibody administered at a dose of 600 mg every three weeks and atezolizumab administered at a dose of 840 mg every two weeks. In some instances, the invention provides methods for assessing responsiveness of a subject or population of subjects having an SCCHN (e.g., a recurrent and/or metastatic SCCHN) to a therapy comprising an anti-TIGIT antagonist antibody and a PD-1 axis binding antagonist (e.g., an anti-PD-L1 antagonist antibody (e.g., atezolizumab) or an anti-PD-1 antagonist antibody (e.g., MDX-1106 (nivolumab) or MK-3475 (pembrolizumab, previously known as lambrolizumab))), by obtaining a tumor sample (e.g., a biopsy) from the subject or population of subjects, detecting the protein expression level of PD-L1 in the tumor sample by an IHC assay using an anti-PD-L1 antibody suitable for staining, and identifying the subject or population of subjects as one who is likely to benefit from a therapy comprising one or more dosing cycles of tiragolumab administered at a dose of 600 mg every three weeks and atezolizumab administered at a dose of 1200 mg every three weeks. In some instances, the method further includes administering to the identified subject or population of subjects the therapy. In some instances, the therapy may further include, or be administered in conjunction with (either separately or together), one or more additional anti-cancer therapeutic agent(s) (e.g., an immunomodulatory agent (e.g., an agent that decreases or inhibits one or more immune co-inhibitory receptors (e.g., one or more immune co-inhibitory receptors selected from TIGIT, PD-L1, PD-1, CTLA-4, LAG3, TIM3, BTLA, and/or VISTA), such as a CTLA-4 antagonist, e.g., an anti-CTLA-4 antagonist antibody (e.g., ipilimumab (YERVOY®)), or an agent that increases or activates one or more immune co-stimulatory receptors (e.g., one or more immune co-stimulatory receptors selected from CD226, OX-40, CD28, CD27, CD137, HVEM, and/or GITR), such as an OX-40 agonist, e.g., an OX-40 agonist antibody), a chemotherapeutic agent, a cytotoxic agent, a growth inhibitory agent, a radiotherapy/radiation therapy, and/or an anti-hormonal agent, such as those recited herein above).

In some instances, the invention provides methods assessing responsiveness of a subject or population of subjects having an SCCHN (e.g., a recurrent and/or metastatic SCCHN) to a therapy comprising an anti-TIGIT antagonist antibody and a PD-1 axis binding antagonist (e.g., an anti-PD-L1 antagonist antibody (e.g., atezolizumab) or an anti-PD-1 antagonist antibody (e.g., MDX-1106 (nivolumab) or MK-3475 (pembrolizumab, previously known as lambrolizumab))), by obtaining a tumor sample (e.g., a biopsy) from the subject or population of subjects, detecting the protein expression level of PD-L1 in the tumor sample by an IHC assay using an anti-PD-L1 antibody suitable for staining, and identifying the subject or population of subjects as one who is likely to benefit from a therapy comprising one or more dosing cycles of an anti-TIGIT antagonist antibody administered at a dose (e.g., a fixed dose) of 600 mg every three weeks and atezolizumab administered at a dose (e.g., a fixed dose) of 1200 mg every three weeks. In some instances, the therapy comprises one or more dosing cycles of an anti-TIGIT antagonist antibody administered at a dose of 600 mg every three weeks and atezolizumab administered at a dose of 1680 mg every four weeks. In some instances, the therapy comprises one or more dosing cycles of an anti-TIGIT antagonist antibody administered at a dose of 600 mg every three weeks and atezolizumab administered at a dose of 840 mg every two weeks. In some instances, the invention provides methods assessing responsiveness of a subject or population of subjects having an SCCHN (e.g., a recurrent and/or metastatic SCCHN) to a therapy comprising an anti-TIGIT antagonist antibody and a PD-1 axis binding antagonist (e.g., an anti-PD-L1 antagonist antibody (e.g., atezolizumab) or an anti-PD-1 antagonist antibody (e.g., MDX-1106 (nivolumab) or MK-3475 (pembrolizumab, previously known as lambrolizumab))), by obtaining a tumor sample (e.g., a biopsy) from the subject or population of subjects, detecting the protein expression level of PD-L1 in the tumor sample by an IHC assay using an anti-PD-L1 antibody suitable for staining, and identifying the subject or population of subjects as one who is likely to benefit from a therapy comprising one or more dosing cycles of tiragolumab administered at a dose of 600 mg every three weeks and atezolizumab administered at a dose of 1200 mg every three weeks. In some instances, the method further includes administering to the identified subject or population of subjects the therapy. In some instances, the therapy may further include, or be administered in conjunction with (either separately or together), one or more additional anti-cancer therapeutic agent(s) (e.g., an immunomodulatory agent (e.g., an agent that decreases or inhibits one or more immune co-inhibitory receptors (e.g., one or more immune co-inhibitory receptors selected from TIGIT, PD-L1, PD-1, CTLA-4, LAG3, TIM3, BTLA, and/or VISTA), such as a CTLA-4 antagonist, e.g., an anti-CTLA-4 antagonist antibody (e.g., ipilimumab (YERVOY®)), or an agent that increases or activates one or more immune co-stimulatory receptors (e.g., one or more immune co-stimulatory receptors selected from CD226, OX-40, CD28, CD27, CD137, HVEM, and/or GITR), such as an OX-40 agonist, e.g., an OX-40 agonist antibody), a chemotherapeutic agent, a cytotoxic agent, a growth inhibitory agent, a radiotherapy/radiation therapy, and/or an anti-hormonal agent, such as those recited herein above).

In some instances, the invention provides methods for optimizing a therapy comprising an anti-TIGIT antagonist antibody and a PD-1 axis binding antagonist (e.g., an anti-PD-L1 antagonist antibody (e.g., atezolizumab) or an anti-PD-1 antagonist antibody (e.g., MDX-1106 (nivolumab) or MK-3475 (pembrolizumab, previously known as lambrolizumab))) in a subject or population of subjects having an SCCHN (e.g., a recurrent and/or metastatic SCCHN), by obtaining a tumor sample (e.g., a biopsy) from the subject or population of subjects, detecting the protein expression level of PD-L1 in the tumor sample by an IHC assay using an anti-PD-L1 antibody suitable for staining, and identifying the subject or population of subjects as one who is likely to benefit from a therapy comprising one or more dosing cycles of an anti-TIGIT antagonist antibody administered at a dose (e.g., a fixed dose) of 600 mg every three weeks and atezolizumab administered at a dose (e.g., a fixed dose) of 1200 mg every three weeks. In some instances, the therapy comprises one or more dosing cycles of an anti-TIGIT antagonist antibody administered at a dose of 600 mg every three weeks and atezolizumab administered at a dose of 1680 mg every four weeks. In some instances, the therapy comprises one or more dosing cycles of an anti-TIGIT antagonist antibody administered at a dose of 600 mg every three weeks and atezolizumab administered at a dose of 840 mg every two weeks. In some instances, the invention provides methods for optimizing a therapy comprising an anti-TIGIT antagonist antibody and a PD-1 axis binding antagonist (e.g., an anti-PD-L1 antagonist antibody (e.g., atezolizumab) or an anti-PD-1 antagonist antibody (e.g., MDX-1106 (nivolumab) or MK-3475 (pembrolizumab, previously known as lambrolizumab))) in a subject or population of subjects having an SCCHN (e.g., a recurrent and/or metastatic SCCHN), by obtaining a tumor sample (e.g., a biopsy) from the subject or population of subjects, detecting the protein expression level of PD-L1 in the tumor sample by an IHC assay using an anti-PD-L1 antibody suitable for staining, and identifying the subject or population of subjects as one who is likely to benefit from a therapy comprising one or more dosing cycles of tiragolumab administered at a dose of 600 mg every three weeks and atezolizumab administered at a dose of 1200 mg every three weeks. In some instances, the method further includes administering to the identified subject or population of subjects the therapy. In some instances, the therapy may further include, or be administered in conjunction with (either separately or together), one or more additional anti-cancer therapeutic agent(s) (e.g., an immunomodulatory agent (e.g., an agent that decreases or inhibits one or more immune co-inhibitory receptors (e.g., one or more immune co-inhibitory receptors selected from TIGIT, PD-L1, PD-1, CTLA-4, LAG3, TIM3, BTLA, and/or VISTA), such as a CTLA-4 antagonist, e.g., an anti-CTLA-4 antagonist antibody (e.g., ipilimumab (YERVOY®)), or an agent that increases or activates one or more immune co-stimulatory receptors (e.g., one or more immune co-stimulatory receptors selected from CD226, OX-40, CD28, CD27, CD137, HVEM, and/or GITR), such as an OX-40 agonist, e.g., an OX-40 agonist antibody), a chemotherapeutic agent, a cytotoxic agent, a growth inhibitory agent, a radiotherapy/radiation therapy, and/or an anti-hormonal agent, such as those recited herein above).

In some instances, the invention provides methods for optimizing a therapy comprising an anti-TIGIT antagonist antibody and a PD-1 axis binding antagonist (e.g., an anti-PD-L1 antagonist antibody (e.g., atezolizumab) or an anti-PD-1 antagonist antibody (e.g., MDX-1106 (nivolumab) or MK-3475 (pembrolizumab, previously known as lambrolizumab))) in a subject or population of subjects having an SCCHN (e.g., a recurrent and/or metastatic SCCHN) by obtaining a tumor sample (e.g., a biopsy) from the subject or population of subjects, detecting the protein expression level of PD-L1 in the tumor sample by an IHC assay using an anti-PD-L1 antibody suitable for staining, and identifying the subject or population of subjects as one who is likely to benefit from a therapy comprising one or more dosing cycles of an anti-TIGIT antagonist antibody administered at a dose (e.g., a fixed dose) of 600 mg every three weeks and atezolizumab administered at a dose (e.g., a fixed dose) of 1200 mg every three weeks. In some instances, the therapy comprises one or more dosing cycles of an anti-TIGIT antagonist antibody administered at a dose of 600 mg every three weeks and atezolizumab administered at a dose of 1680 mg every four weeks. In some instances, the therapy comprises one or more dosing cycles of an anti-TIGIT antagonist antibody administered at a dose of 600 mg every three weeks and atezolizumab administered at a dose of 840 mg every two weeks. In some instances, the invention provides methods for optimizing a therapy comprising an anti-TIGIT antagonist antibody and a PD-1 axis binding antagonist (e.g., an anti-PD-L1 antagonist antibody (e.g., atezolizumab) or an anti-PD-1 antagonist antibody (e.g., MDX-1106 (nivolumab) or MK-3475 (pembrolizumab, previously known as lambrolizumab))) in a subject or population of subjects having an SCCHN (e.g., a recurrent and/or metastatic SCCHN) by obtaining a tumor sample (e.g., a biopsy) from the subject or population of subjects, detecting the protein expression level of PD-L1 in the tumor sample by an IHC assay using an anti-PD-L1 antibody suitable for staining, and identifying the subject or population of subjects as one who is likely to benefit from a therapy comprising one or more dosing cycles of tiragolumab administered at a dose of 600 mg every three weeks and atezolizumab administered at a dose of 1200 mg every three weeks. In some instances, the method further includes administering to the identified subject or population of subjects the therapy. In some instances, the therapy may further include, or be administered in conjunction with (either separately or together), one or more additional anti-cancer therapeutic agent(s) (e.g., an immunomodulatory agent (e.g., an agent that decreases or inhibits one or more immune co-inhibitory receptors (e.g., one or more immune co-inhibitory receptors selected from TIGIT, PD-L1, PD-1, CTLA-4, LAG3, TIM3, BTLA, and/or VISTA), such as a CTLA-4 antagonist, e.g., an anti-CTLA-4 antagonist antibody (e.g., ipilimumab (YERVOY®)), or an agent that increases or activates one or more immune co-stimulatory receptors (e.g., one or more immune co-stimulatory receptors selected from CD226, OX-40, CD28, CD27, CD137, HVEM, and/or GITR), such as an OX-40 agonist, e.g., an OX-40 agonist antibody), a chemotherapeutic agent, a cytotoxic agent, a growth inhibitory agent, a radiotherapy/radiation therapy, and/or an anti-hormonal agent, such as those recited herein above).

In some instances, the invention provides methods optimizing a therapy comprising an anti-TIGIT antagonist antibody and a PD-1 axis binding antagonist (e.g., an anti-PD-L1 antagonist antibody (e.g., atezolizumab) or an anti-PD-1 antagonist antibody (e.g., MDX-1106 (nivolumab) or MK-3475 (pembrolizumab, previously known as lambrolizumab))) in a subject or population of subjects having an SCCHN (e.g., a recurrent and/or metastatic SCCHN) by obtaining a tumor sample (e.g., a biopsy) from the subject or population of subjects, detecting the protein expression level of PD-L1 in the tumor sample by an IHC assay using an anti-PD-L1 antibody suitable for staining, and identifying the subject or population of subjects as one who is likely to benefit from a therapy comprising one or more dosing cycles of an anti-TIGIT antagonist antibody administered at a dose (e.g., a fixed dose) of 600 mg every three weeks and atezolizumab administered at a dose (e.g., a fixed dose) of 1200 mg every three weeks. In some instances, the therapy comprises one or more dosing cycles of an anti-TIGIT antagonist antibody administered at a dose of 600 mg every three weeks and atezolizumab administered at a dose of 1680 mg every four weeks. In some instances, the therapy comprises one or more dosing cycles of an anti-TIGIT antagonist antibody administered at a dose of 600 mg every three weeks and atezolizumab administered at a dose of 840 mg every two weeks. In some instances, the invention provides methods optimizing a therapy comprising an anti-TIGIT antagonist antibody and a PD-1 axis binding antagonist (e.g., an anti-PD-L1 antagonist antibody (e.g., atezolizumab) or an anti-PD-1 antagonist antibody (e.g., MDX-1106 (nivolumab) or MK-3475 (pembrolizumab, previously known as lambrolizumab))) in a subject or population of subjects having an SCCHN (e.g., a recurrent and/or metastatic SCCHN) by obtaining a tumor sample (e.g., a biopsy) from the subject or population of subjects, detecting the protein expression level of PD-L1 in the tumor sample by an IHC assay using an anti-PD-L1 antibody suitable for staining, and identifying the subject or population of subjects as one who is likely to benefit from a therapy comprising one or more dosing cycles of tiragolumab administered at a dose of 600 mg every three weeks and atezolizumab administered at a dose of 1200 mg every three weeks. In some instances, the method further includes administering to the identified subject or population of subjects the therapy. In some instances, the therapy may further include, or be administered in conjunction with (either separately or together), one or more additional anti-cancer therapeutic agent(s) (e.g., an immunomodulatory agent (e.g., an agent that decreases or inhibits one or more immune co-inhibitory receptors (e.g., one or more immune co-inhibitory receptors selected from TIGIT, PD-L1, PD-1, CTLA-4, LAG3, TIM3, BTLA, and/or VISTA), such as a CTLA-4 antagonist, e.g., an anti-CTLA-4 antagonist antibody (e.g., ipilimumab (YERVOY®)), or an agent that increases or activates one or more immune co-stimulatory receptors (e.g., one or more immune co-stimulatory receptors selected from CD226, OX-40, CD28, CD27, CD137, HVEM, and/or GITR), such as an OX-40 agonist, e.g., an OX-40 agonist antibody), a chemotherapeutic agent, a cytotoxic agent, a growth inhibitory agent, a radiotherapy/radiation therapy, and/or an anti-hormonal agent, such as those recited herein above).

In some instances, the method further includes administering to the identified subject or population of subjects the therapy. In some instances, the therapy may further include, or be administered in conjunction with (either separately or together), one or more additional anti-cancer therapeutic agent(s) (e.g., an immunomodulatory agent (e.g., an agent that decreases or inhibits one or more immune co-inhibitory receptors (e.g., one or more immune co-inhibitory receptors selected from TIGIT, PD-L1, PD-1, CTLA-4, LAG3, TIM3, BTLA, and/or VISTA), such as a CTLA-4 antagonist, e.g., an anti-CTLA-4 antagonist antibody (e.g., ipilimumab (YERVOY®)), or an agent that increases or activates one or more immune co-stimulatory receptors (e.g., one or more immune co-stimulatory receptors selected from CD226, OX-40, CD28, CD27, CD137, HVEM, and/or GITR), such as an OX-40 agonist, e.g., an OX-40 agonist antibody), a chemotherapeutic agent, a cytotoxic agent, a growth inhibitory agent, a radiotherapy/radiation therapy, and/or an anti-hormonal agent, such as those recited herein above).

In some instances, the method further includes administering to the identified subject or population of subjects the therapy. In some instances, the therapy may further include, or be administered in conjunction with (either separately or together), one or more additional anti-cancer therapeutic agent(s) (e.g., an immunomodulatory agent (e.g., an agent that decreases or inhibits one or more immune co-inhibitory receptors (e.g., one or more immune co-inhibitory receptors selected from TIGIT, PD-L1, PD-1, CTLA-4, LAG3, TIM3, BTLA, and/or VISTA), such as a CTLA-4 antagonist, e.g., an anti-CTLA-4 antagonist antibody (e.g., ipilimumab (YERVOY®)), or an agent that increases or activates one or more immune co-stimulatory receptors (e.g., one or more immune co-stimulatory receptors selected from CD226, OX-40, CD28, CD27, CD137, HVEM, and/or GITR), such as an OX-40 agonist, e.g., an OX-40 agonist antibody), a chemotherapeutic agent, a cytotoxic agent, a growth inhibitory agent, a radiotherapy/radiation therapy, and/or an anti-hormonal agent, such as those recited herein above).

In some instances, in any of the diagnostic methods or uses described herein, the SCCHN is a recurrent and/or metastatic SCCHN. The cancer may be at an early or late stage. In some instances, the therapy is a first-line therapy. In some instances, the subject or population of subjects has not received prior therapy. In some instances, the prior therapy is a prior systemic therapy for recurrent and/or metastatic disease.

Cancer Characterization and Selection

In any of the methods, uses, or compositions for use described herein, the cancer may be SCCHN (e.g., a recurrent and/or metastatic SCCHN). In some instances, the SCCHN is a recurrent and/or metastatic SCCHN. In some instances, the SCCHN is PD-L1-positive. In some instances, the SCCHN is HPV-positive. In some instances, the SCCHN is HPV-negative. In some instances, the SCCHN is PD-L1-positive and HPV-positive. In some instances, the SCCHN is PD-L1-positive and HPV-negative. In some instances, the subject or population of subjects has not received prior therapy. In some instances, the prior therapy is a prior systemic therapy for recurrent and/or metastatic disease.

In some instances, in any of the methods, uses, or compositions for use described herein, the subject or population of subjects has a PD-L1 selected tumor (e.g., a tumor with a detectable expression level of PD-L1 (e.g., a detectable protein expression level of PD-L1 (e.g., a tumor-associated immune-cell (TIC) of greater than or equal to 5%))). In some instances, a sample (e.g., a tumor sample (e.g., a tumor tissue sample)) obtained from the subject or population of subjects has a detectable protein expression level of PD-L1 (e.g., a TIC of greater than or equal to 5%). In some instances, the PD-L1 selected tumor has a detectable expression level of PD-L1 (e.g., a detectable protein expression level of PD-L1 (e.g., a TIC of greater than or equal to 5%)). In some instances, the detectable expression level of PD-L1 is a detectable protein expression level of PD-L1 (e.g., a TIC of greater than or equal to 5%). In some instances, the detectable protein expression level of PD-L1 is a TIC of greater than or equal to about 5% (e.g., 5%, 6%, 7%, 8%, 9%, 10%, 11%, 12%, 13%, 14%, 15%, 16%, 17%, 18%, 19%, 20%, 25%, 30%, 35%, 40%, 45%, 50%, 60%, 70%, 75%, 80%, 90% or more). In some instances, the detectable protein expression level of PD-L1 is a TIC of greater than or equal to 5% and less than 20% (e.g., 5%, 6%, 7%, 8%, 9%, 10%, 11%, 12%, 13%, 14%, 15%, 16%, 17%, 18%, 19%, or 20%). In some instances, a TIC of greater than or equal to 5% and less than 20% is defined as PD-L1 low. In some instances, the detectable protein expression level of PD-L1 is a TIC of greater than or equal to 20% (e.g., 20%, 21%, 22%, 23%, 24%, 25%, 26%, 27%, 28%, 29%, 30%, 31%, 32%, 33%, 34%, 35%, 40%, 45%, 50%, 55%, 60%, 65%, 70%, 75%, 80%, 85%, 90%, 95%, 99%, or 100%). In some instances, a TIC of greater than or equal to 20% is defined as PD-L1 high. In some instances, the detectable protein expression level of PD-L1 is a TIC of greater than or equal to about 10% (e.g., 10%, 11%, 12%, 13%, 14%, 15%, 16%, 17%, 18%, 1 9%, 20%, 25%, 30%, 35%, 40%, 45%, 50%, 60%, 70%, 75%, 80%, 90% or more). In some instances, the detectable protein expression level of PD-L1 is a TIC of greater than or equal to 10% and less than 50% (e.g., 10%, 11%, 12%, 13%, 14%, 15%, 16%, 17%, 18%, 19%, 20%, 21%, 22%, 23%, 24%, 25%, 30%, 35%, 40%, 45%, 46%, 47%, 48%, 49%, 49.5%, 49.9%, or 49.99%). In some instances, the detectable protein expression level of PD-L1 is a TIC of greater than or equal to 50% (e.g., 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 95%, 99%, or 99.99%).

In some instances, the IHC assay uses the anti-PD-L1 antibody SP263, SP142, 22C3, or 28-8. In some instances, the IHC assay uses anti-PD-L1 antibody SP263. In some instances, the TIC has been determined (e.g., using the Ventana (SP263) PD-L1 IHC assay) to be greater than, or equal to, 5%. In some instances, the TIC has been determined (e.g., using the Ventana (SP263) PD-L1 IHC assay) to be greater than, or equal to, 5% and less than 20%. In some instances, the TIC has been determined (e.g., using the Ventana (SP263) PD-L1 IHC assay) to be greater than, or equal to, 20%. In some instances, the TIC has been determined (e.g., using the Ventana (SP263) PD-L1 IHC assay) to be greater than, or equal to, 10%. In some instances, the TIC has been determined (e.g., using the Ventana (SP263) PD-L1 IHC assay) to be greater than, or equal to, 10% and less than 50%. In some instances, the TIC has been determined (e.g., using the Ventana (SP263) PD-L1 IHC assay) to be greater than, or equal to, 50%.

In some instances, in any of the methods, uses, or compositions for use described herein, a sample (e.g., a tumor sample) obtained from the subject or population of subjects has a detectable nucleic acid expression level of PD-L1. In some instances, the detectable nucleic acid expression level of PD-L1 has been determined by RNA-seq, RT-qPCR, qPCR, multiplex qPCR or RT-qPCR, microarray analysis, SAGE, MassARRAY technique, ISH, or a combination thereof. In some instances, the sample is selected from the group consisting of a tissue sample, a whole blood sample, a serum sample, and a plasma sample. In some instances, the tissue sample is a tumor sample. In some instances, the tumor sample comprises tumor-infiltrating immune cells, tumor cells, stromal cells, and any combinations thereof.

In some instances, in any of the methods, uses, or compositions for use described herein, the subject or population of subjects has a tumor with a detectable HPV. In some instances, in any of the methods, uses, or compositions for use described herein, the subject or population of subjects has a tumor without detectable HPV. In some instances, the HPV is detected directly or indirectly. In some instances, the HPV is detected by a protein expression level (e.g., p16 or an HPV viral protein). In some instances, the HPV is detected by a nucleic acid expression level. In some instances, HPV status is determined by p16 IHC, in situ hybridization, or by PCR. In some instances, the HPV is HPV16. In some instances, the HPV is HPV18.

Responses to Treatment

In some embodiments of any of the methods described herein, a subject or population of subjects' response to the therapy can be characterized by one or more measures. In some embodiments, the treatment results in a CR or a PR. In some instances, any of the methods and uses for treating an SCCHN in a subject or population of subjects can result in an increase in the objective response rate (ORR) of the subject or population of subjects. In some instances, any of the methods and uses for treating an SCCHN in a subject or population of subjects can result in an increase in the PFS, duration of response (DOR), and/or OS of the subject or population of subjects.

In some instances, in any of the methods, uses, or compositions for use described herein, administration of the anti-TIGIT antagonist antibody and PD-1 axis binding antagonist (e.g., anti-PD-L1 antagonist antibody (e.g., atezolizumab) or anti-PD-1 antagonist antibody (e.g., MDX-1106 (nivolumab) or MK-3475 (pembrolizumab, previously known as lambrolizumab))) results in a clinical response. In some instances, the clinical response is an increase in the ORR of the subject or population of subjects as compared to a reference ORR. In some instances, the reference ORR is the median ORR of a population of subjects who have received a treatment comprising a PD-1 axis binding antagonist (e.g., an anti-PD-L1 antagonist antibody (e.g., atezolizumab) or an anti-PD-1 antagonist antibody (e.g., MDX-1106 (nivolumab) or MK-3475 (pembrolizumab, previously known as lambrolizumab))) without an anti-TIGIT antagonist antibody. In some instances, the reference ORR is at least about 19% (e.g., between about 19% and about 80%, e.g., between about 19% and about 60% (e.g., 20%, 21%, 22%, 23%, 24%, 25%, 30%, 35%, 40%, 45%, 50%, 55%, or 60%)). In some instances, the reference ORR is at least about 36% (e.g., between about 36% and about 80%, e.g., between about 36% and about 60% (e.g., 36%, 37%, 38%, 39%, 40%, 45%, 50%, 55%, or 60%)). In some instances, the reference ORR is at least about 19% to about 36%. In some instances, the clinical response is an increase in the PFS of the subject or population of subjects as compared to a reference PFS time. In some instances, wherein the reference PFS time is the median PFS time of a population of subjects who have received a treatment comprising a PD-1 axis binding antagonist (e.g., an anti-PD-L1 antagonist antibody (e.g., atezolizumab) or an anti-PD-1 antagonist antibody (e.g., MDX-1106 (nivolumab) or MK-3475 (pembrolizumab, previously known as lambrolizumab))) without an anti-TIGIT antagonist antibody. In some instances, the clinical response is an increase in the DOR of the subject or population of subjects compared to a reference DOR time. In some instances, wherein the reference DOR time is the median DOR time of a population of subjects who have received a treatment comprising a PD-1 axis binding antagonist (e.g., an anti-PD-L1 antagonist antibody (e.g., atezolizumab) or an anti-PD-1 antagonist antibody (e.g., MDX-1106 (nivolumab) or MK-3475 (pembrolizumab, previously known as lambrolizumab))) without an anti-TIGIT antagonist antibody. In some instances, the reference DOR time is at least about 4 months (e.g., between about 4 months and about 42 months (e.g., between about 6 months and about 30 months (e.g., 6 months, 7 months, 8 months, 9 months, 10 months, 11 months, 12 months, 13 months, 14 months, 15 months, 16 months, 17 months, 18 months, 19 months, 20 months, 21 months, 22 months, 23 months, 24 months, 25 months, 26 months, 27 months, 28 months, 29 months, or 30 months))). In some instances, the reference DOR time is at least about 6.7 months (e.g., at least about 6.7 months, 6.8 months, 6.9 months, 7.0 months, 8.0 months, 9.0 months, 10.0 months, 11.0 months, or 12.0 months). In some instances, the reference DOR time is at least about 6.7 months to about 23.4 months. In some instances, the reference DOR time is at least about 23.4 months (e.g., at least about 23.4 months, 23.5 months, 23.6 months, 23.7 months, 23.8 months, 23.9 months, 24 months, 25 months, 26 months, 28 months, 30 months, or 32 months). In some instances, the clinical response is an increase in the OS.

In some instances, the treatment results in an ORR of greater than 15% (e.g., 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 50%, 55%, 60%, 65%, 70%, 75%, 80%, 90% or more). In some instances, the treatment results in an ORR that is greater than a reference ORR. In some instances, the reference ORR is 19%. In some instances, the reference ORR is 24.4%. In some instances, the reference ORR is 28.8%. In some instances, the reference ORR is 35%.

In some instances, the treatment results in an increase in PFS, DOR, or OS of the subject or population of subjects, e.g., as compared to treatment with the PD-1 axis binding antagonist without the anti-TIGIT antagonist antibody or as compared to treatment with the anti-TIGIT antagonist antibody without the PD-1 axis binding antagonist.

In some embodiments, OS is measured as the period of time from the start of treatment to death. In some instances, the treatment extends the OS of the subject or population of subjects by at least about 2 months (e.g., by 2-120 months, by 3-110 months, by 4-100 months, by 5-80 months, by 6-60 months, by 7-48 months, by 8-36 months, or by 10-24 months, e.g., by at least about 2 months, 2.1 months, 2.2 months, 2.3 months, 2.4 months, 2.5 months, 2.6 months, 2.7 months, 2.8 months, 2.9 months, 3.0 months, 3.1 months, 3.2 months, 3.3 months, 3.4 months, 3.5 months, 3.6 months, 3.7 months, 3.8 months, 3.9 months, 4.0 months, 4.1 months, 4.2 months, 4.3 months, 4.4 months, 4.5 months, 4.6 months, 4.7 months, 4.8 months, 4.9 months, 5.0 months, 5.5 months, 6.0 months, 6.5 months, 7.0 months, 7.5 months, 8.0 months, 8.5 months, 9.0 months, 9.5 months, 10 months, 10.5 months, 11 months, 11.5 months, 12 months, 13 months, 14 months, 15 months, 16 months, 17 months, 18 months, 19 months, 20 months, 21 months, 22 months, 23 months, 24 months, 25 months, 26 months, 27 months, 28 months, 29 months, 30 months, 31 months, 32 months, 33 months, 34 months, 35 months, or 36 months). In some instances, the treatment extends the OS of the subject or population of subjects by at least about 3.3 months (e.g., by 3.3-120 months, by 4-100 months, by 5-80 months, by 6-60 months, by 7-48 months, by 8-36 months, or by 10-24 months, e.g., by at least about 3.3 months, 3.4 months, 3.5 months, 3.6 months, 3.7 months, 3.8 months, 3.9 months, 4.0 months, 4.1 months, 4.2 months, 4.3 months, 4.4 months, 4.5 months, 4.6 months, 4.7 months, 4.8 months, 4.9 months, 5.0 months, 5.5 months, 6.0 months, 6.5 months, 7.0 months, 7.5 months, 8.0 months, 8.5 months, 9.0 months, 9.5 months, 10 months, 10.5 months, 11 months, 11.5 months, 12 months, 13 months, 14 months, 15 months, 16 months, 17 months, 18 months, 19 months, 20 months, 21 months, 22 months, 23 months, 24 months, 25 months, 26 months, 27 months, 28 months, 29 months, 30 months, 31 months, 32 months, 33 months, 34 months, 35 months, or 36 months). In some instances, the treatment extends the OS of the subject or population of subjects by at least about 5.3 months (e.g., by 5.3-120, by 6-60 months, by 7-48 months, by 8-36 months, or by 10-24 months, e.g., by at least about 5.3 months, 5.5 months, 6.0 months, 6.5 months, 7.0 months, 7.5 months, 8.0 months, 8.5 months, 9.0 months, 9.5 months, 10 months, 10.5 months, 11 months, 11.5 months, 12 months, 13 months, 14 months, 15 months, 16 months, 17 months, 18 months, 19 months, 20 months, 21 months, 22 months, 23 months, 24 months, 25 months, 26 months, 27 months, 28 months, 29 months, 30 months, 31 months, 32 months, 33 months, 34 months, 35 months, or 36 months). In some instances, the treating results in an increase in OS as compared to a reference OS time. In some instances, the reference OS time is at least about 8 months (e.g., 8 months, 9 months, 10 months, 11 months, 12 months, 13 months, 14 months, 15 months, 16 months, 17 months, or 18 months). In some instances, the reference OS time is at least about 14.9 months (e.g., 15 months, 16 months, 17 months, 18 months, 19 months, or 20 months). In some instances, the reference OS time is at least about 11.6 months to about 14.9 months.

In some embodiments, a treatment described herein extends the PFS of the subject or population of subjects by at least about 2.4 months (e.g., by 2.4-120 months, by 2.5-100 months, by 3.0-80 months, by 4.0-60 months, by 5.0-48 months, by 6.0-36 months, by 8.0-24 months, or by 10-12 months, e.g., by at least about 2.4 months, 2.5 months, 2.6 months, 2.7 months, 2.8 months, 2.9 months, 3.0 months, 3.1 months, 3.2 months, 3.3 months, 3.4 months, 3.5 months, 3.6 months, 3.7 months, 3.8 months, 3.9 months, 4.0 months, 4.1 months, 4.2 months, 4.3 months, 4.4 months, 4.5 months, 4.6 months, 4.7 months, 4.8 months, 4.9 months, 5.0 months, 5.5 months, 6.0 months, 6.5 months, 7.0 months, 7.5 months, 8.0 months, 8.5 months, 9.0 months, 9.5 months, 10 months, 10.5 months, 11 months, 11.5 months, 12 months, 13 months, 14 months, 15 months, 16 months, 17 months, 18 months, 19 months, 20 months, 21 months, 22 months, 23 months, 24 months, 25 months, 26 months, 27 months, 28 months, 29 months, 30 months, 31 months, 32 months, 33 months, 34 months, 35 months, or 36 months). In some embodiments, the treatment extends the PFS of the subject or population of subjects by at least about 4 months (e.g., by 4-120 months, by 5-100 months, by 6-80 months, by 7-60 months, by 8-48 months, by 9-36 months, or by 10-24 months, e.g., by at least about 4.0 months, 4.1 months, 4.2 months, 4.3 months, 4.4 months, 4.5 months, 4.6 months, 4.7 months, 4.8 months, 4.9 months, 5.0 months, 5.5 months, 6.0 months, 6.5 months, 7.0 months, 7.5 months, 8.0 months, 8.5 months, 9.0 months, 9.5 months, 10 months, 10.5 months, 11 months, 11.5 months, 12 months, 13 months, 14 months, 15 months, 16 months, 17 months, 18 months, 19 months, 20 months, 21 months, 22 months, 23 months, 24 months, 25 months, 26 months, 27 months, 28 months, 29 months, 30 months, 31 months, 32 months, 33 months, 34 months, 35 months, or 36 months). In some embodiments, the treatment extends the PFS of the subject or population of subjects by at least about 2 months (e.g., by 2-120 months, by 3-100 months, by 4-80 months, by 6-60 months, by 8-48 months, by 9-36 months, or by 10-24 months, e.g., by at least about 2.0 months, 2.1 months, 2.2 months, 2.3 months, 2.4 months, 2.5 months, 2.6 months, 2.7 months, 2.8 months, 2.9 months, 3.0 months, 3.1 months, 3.2 months, 3.3 months, 3.4 months, 3.5 months, 3.6 months, 3.7 months, 3.8 months, 3.9 months, 4.0 months, 4.1 months, 4.2 months, 4.3 months, 4.4 months, 4.5 months, 4.6 months, 4.7 months, 4.8 months, 4.9 months, 5.0 months, 5.5 months, 6.0 months, 6.5 months, 7.0 months, 7.5 months, 8.0 months, 8.5 months, 9.0 months, 9.5 months, 10 months, 10.5 months, 11 months, 11.5 months, 12 months, 13 months, 14 months, 15 months, 16 months, 17 months, 18 months, 19 months, 20 months, 21 months, 22 months, 23 months, 24 months, 25 months, 26 months, 27 months, 28 months, 29 months, 30 months, 31 months, 32 months, 33 months, 34 months, 35 months, or 36 months).

In some embodiments, a treatment described herein extends the DOR of the subject or population of subjects by at least about 2.4 months (e.g., by 2.4-120 months, by 2.5-100 months, by 3.0-80 months, by 4.0-60 months, by 5.0-48 months, by 6.0-36 months, by 8.0-24 months, or by 10-12 months, e.g., by at least about 2.4 months, 2.5 months, 2.6 months, 2.7 months, 2.8 months, 2.9 months, 3.0 months, 3.1 months, 3.2 months, 3.3 months, 3.4 months, 3.5 months, 3.6 months, 3.7 months, 3.8 months, 3.9 months, 4.0 months, 4.1 months, 4.2 months, 4.3 months, 4.4 months, 4.5 months, 4.6 months, 4.7 months, 4.8 months, 4.9 months, 5.0 months, 5.5 months, 6.0 months, 6.5 months, 7.0 months, 7.5 months, 8.0 months, 8.5 months, 9.0 months, 9.5 months, 10 months, 10.5 months, 11 months, 11.5 months, 12 months, 13 months, 14 months, 15 months, 16 months, 17 months, 18 months, 19 months, 20 months, 21 months, 22 months, 23 months, 24 months, 25 months, 26 months, 27 months, 28 months, 29 months, 30 months, 31 months, 32 months, 33 months, 34 months, 35 months, or 36 months). In some embodiments, the treatment extends the DOR of the subject or population of subjects by at least about 4 months (e.g., by 4-120 months, by 5-100 months, by 6-80 months, by 7-60 months, by 8-48 months, by 9-36 months, or by 10-24 months, e.g., by at least about 4.0 months, 4.1 months, 4.2 months, 4.3 months, 4.4 months, 4.5 months, 4.6 months, 4.7 months, 4.8 months, 4.9 months, 5.0 months, 5.5 months, 6.0 months, 6.5 months, 7.0 months, 7.5 months, 8.0 months, 8.5 months, 9.0 months, 9.5 months, 10 months, 10.5 months, 11 months, 11.5 months, 12 months, 13 months, 14 months, 15 months, 16 months, 17 months, 18 months, 19 months, 20 months, 21 months, 22 months, 23 months, 24 months, 25 months, 26 months, 27 months, 28 months, 29 months, 30 months, 31 months, 32 months, 33 months, 34 months, 35 months, or 36 months). In some embodiments, the treatment extends the DOR of the subject or population of subjects by at least about 2 months (e.g., by 2-120 months, by 3-100 months, by 4-80 months, by 6-60 months, by 8-48 months, by 9-36 months, or by 10-24 months, e.g., by at least about 2.0 months, 2.1 months, 2.2 months, 2.3 months, 2.4 months, 2.5 months, 2.6 months, 2.7 months, 2.8 months, 2.9 months, 3.0 months, 3.1 months, 3.2 months, 3.3 months, 3.4 months, 3.5 months, 3.6 months, 3.7 months, 3.8 months, 3.9 months, 4.0 months, 4.1 months, 4.2 months, 4.3 months, 4.4 months, 4.5 months, 4.6 months, 4.7 months, 4.8 months, 4.9 months, 5.0 months, 5.5 months, 6.0 months, 6.5 months, 7.0 months, 7.5 months, 8.0 months, 8.5 months, 9.0 months, 9.5 months, 10 months, 10.5 months, 11 months, 11.5 months, 12 months, 13 months, 14 months, 15 months, 16 months, 17 months, 18 months, 19 months, 20 months, 21 months, 22 months, 23 months, 24 months, 25 months, 26 months, 27 months, 28 months, 29 months, 30 months, 31 months, 32 months, 33 months, 34 months, 35 months, or 36 months).

In some instances, the treatment results in an improvement in time to confirmed deterioration (TTCD) in patient-reported physical functioning, as measured by the Patient-Reported Outcomes Measurement Information System® (PROMIS®) Item Bank v2.0-Physical Functioning-Short Form 10b. In some instances, the treatment results in an improvement from baseline in physical functioning, fatigue, and/or pain, assessed through PROMIS® Item Bank v2.0-Physical Functioning-Short Form 10b, PROMIS® Item Bank v1.0-Fatigue-Short Form 4a, PROMIS® Item Bank v1.0-Pain Interference Short Form 4a, and PROMIS® Numeric Rating Scale v1.0-Pain Intensity 1 a.

F. Therapeutic Methods and Uses Relating to Liver Cancer

Liver Cancer

Liver cancer is the fifth most common cancer and the second most frequent cause of cancer-related death globally, with 854,000 new cases and 810,000 deaths per year. Upon diagnosis, most patients with primary liver cancer present with advanced disease, a stage when treatment with curative therapies is not recommended. The World Health Organization estimates that more than 1 million people will die from liver cancer in 2030, highlighting a significant global public health issue.

Hepatocellular carcinoma (HCC) is the most prevalent form of primary liver cancer and represents approximately 90% of all primary hepatic malignancies. HCC is a highly lethal disease with the highest mortality-to-incidence rate ratio of 0.98 of any solid tumor. Up to 80% of patients first presenting with HCC have advanced unresectable or metastatic disease because of the late appearance of symptoms. In the United States, the 5-year OS rate of patients with HCC is 17% and falls substantially to only 3% if present with distant metastasis.

Thus, there is an unmet need in the field for the development of efficacious immunotherapies for the treatment of liver cancer, e.g., HCC, e.g., locally advanced HCC, metastatic HCC, or unresectable HCC.

Methods and Uses for Treating Liver Cancer

Provided herein are methods and uses for treating or delaying progression of liver cancer, e.g., hepatocellular carcinoma (HCC), including locally advanced or metastatic and/or unresectable HCC, in a subject or population of subjects comprising administering to the subject or population of subjects a treatment regimen comprising an effective amount of a PD-1 axis binding antagonist (e.g., an anti-PD-L1 antagonist antibody (e.g., atezolizumab)), and an anti-TIGIT antagonist antibody (e.g., tiragolumab), wherein the subject or population of subjects has not previously received systemic treatment for the liver cancer (e.g., HCC). Also provided herein are methods and uses for treating or delaying progression of liver cancer, e.g., HCC, including locally advanced or metastatic and/or unresectable HCC in a subject or population of subjects comprising administering to the subject or population of subjects a treatment regimen comprising an effective amount of a PD-1 axis binding antagonist (e.g., an anti-PD-L1 antagonist antibody (e.g., atezolizumab)), a VEGF antagonist (e.g., an anti-VEGF antibody (e.g., bevacizumab)), and an anti-TIGIT antagonist antibody (e.g., tiragolumab). In some embodiments, the treatment regimen comprises one or more dosing cycles of an effective amount of a PD-1 axis binding antagonist (e.g., an anti-PD-L1 antagonist antibody (e.g., atezolizumab)) and an anti-TIGIT antagonist antibody (e.g., tiragolumab). In other embodiments, the treatment regimen comprises one or more dosing cycles of an effective amount of a PD-1 axis binding antagonist (e.g., an anti-PD-L1 antagonist antibody (e.g., atezolizumab)), a VEGF antagonist (e.g., an anti-VEGF antibody (e.g., bevacizumab)), and an anti-TIGIT antagonist antibody (e.g., tiragolumab).

Also provided herein are methods of enhancing immune function in a subject or population of subjects having liver cancer (e.g., HCC, including locally advanced or metastatic and/or unresectable HCC) comprising administering to the subject or population of subjects a treatment regimen comprising an effective amount of a PD-1 axis binding antagonist (e.g., an anti-PD-L1 antagonist antibody (e.g., atezolizumab)) and an anti-TIGIT antagonist antibody (e.g., tiragolumab). In some embodiments, the treatment regimen comprises one or more dosing cycles of an effective amount of a PD-1 axis binding antagonist (e.g., an anti-PD-L1 antagonist antibody (e.g., atezolizumab)) and an anti-TIGIT antagonist antibody (e.g., tiragolumab).

For example, provided herein are methods and uses for treating or delaying progression of liver cancer (e.g., HCC, including locally advanced or metastatic and/or unresectable HCC) in a subject or population of subjects comprising administering to the subject or population of subjects a treatment regimen comprising an effective amount of a PD-1 axis binding antagonist (e.g., an anti-PD-L1 antagonist antibody (e.g., atezolizumab)) and an anti-TIGIT antagonist antibody (e.g., tiragolumab). In some embodiments, the treatment regimen comprises one or more dosing cycles of an effective amount of a PD-1 axis binding antagonist (e.g., an anti-PD-L1 antagonist antibody (e.g., atezolizumab)) and an anti-TIGIT antagonist antibody (e.g., tiragolumab).

Also provided herein is a pharmaceutical composition comprising a PD-1 axis binding antagonist (e.g., an anti-PD-L1 antagonist antibody (e.g., atezolizumab)) for use in treatment of liver cancer (e.g., HCC, including locally advanced or metastatic and/or unresectable HCC) in a subject or population of subjects, wherein the treatment comprises a treatment regimen comprising administration of the PD-1 axis binding antagonist in combination with an anti-TIGIT antagonist antibody (e.g., tiragolumab). In some embodiments, the treatment regimen comprises one or more dosing cycles of an effective amount of a PD-1 axis binding antagonist (e.g., an anti-PD-L1 antagonist antibody (e.g., atezolizumab)) and an anti-TIGIT antagonist antibody (e.g., tiragolumab).

Also provided herein is a pharmaceutical composition comprising a PD-1 axis binding antagonist (e.g., an anti-PD-L1 antagonist antibody (e.g., atezolizumab)) for use in treatment of liver cancer (e.g., HCC, including locally advanced or metastatic and/or unresectable HCC) in a subject or population of subjects, wherein the treatment comprises a treatment regimen comprising administration of the PD-1 axis binding antagonist in combination with a VEGF antagonist (e.g., an anti-VEGF antibody (e.g., bevacizumab)) and an anti-TIGIT antagonist antibody (e.g., tiragolumab). In some embodiments, the treatment regimen comprises one or more dosing cycles of an effective amount of a PD-1 axis binding antagonist (e.g., an anti-PD-L1 antagonist antibody (e.g., atezolizumab)), a VEGF antagonist (e.g., an anti-VEGF antibody (e.g., bevacizumab)), and an anti-TIGIT antagonist antibody (e.g., tiragolumab).

In another example, provided herein is a pharmaceutical composition comprising a VEGF antagonist (e.g., an anti-VEGF antibody (e.g., bevacizumab)) for use in treatment of liver cancer (e.g., HCC, including locally advanced or metastatic and/or unresectable HCC) in a subject or population of subjects, wherein the treatment comprises a treatment regimen comprising administration of the VEGF antagonist in combination with a PD-1 axis binding antagonist (e.g., an anti-PD-L1 antagonist antibody (e.g., atezolizumab)) and an anti-TIGIT antagonist antibody (e.g., tiragolumab). In some embodiments, the treatment regimen comprises one or more dosing cycles of an effective amount of a PD-1 axis binding antagonist (e.g., an anti-PD-L1 antagonist antibody (e.g., atezolizumab)), a VEGF antagonist (e.g., an anti-VEGF antibody (e.g., bevacizumab)), and an anti-TIGIT antagonist antibody (e.g., tiragolumab).

In another example, provided herein is a pharmaceutical composition comprising an anti-TIGIT antagonist antibody (e.g., tiragolumab) for use in treatment of liver cancer (e.g., HCC, including locally advanced or metastatic and/or unresectable HCC) in a subject or population of subjects, wherein the treatment comprises a treatment regimen comprising administration of the anti-TIGIT antagonist antibody in combination with a PD-1 axis binding antagonist (e.g., an anti-PD-L1 antagonist antibody (e.g., atezolizumab)) and a VEGF antagonist (e.g., an anti-VEGF antibody (e.g., bevacizumab)). In some embodiments, the treatment regimen comprises one or more dosing cycles of an effective amount of a PD-1 axis binding antagonist (e.g., an anti-PD-L1 antagonist antibody (e.g., atezolizumab)), a VEGF antagonist (e.g., an anti-VEGF antibody (e.g., bevacizumab)), and an anti-TIGIT antagonist antibody (e.g., tiragolumab).

In some embodiments of any of the methods, uses, or pharmaceutical compositions for use described herein, the liver cancer may be at an early or late stage. In some embodiments, the liver cancer is an HCC (e.g., locally advanced or metastatic and/or unresectable HCC). In some instances, the liver cancer is a locally advanced or metastatic and/or unresectable HCC. In some embodiments, the liver cancer is high-risk liver cancer (e.g., high-risk locally advanced or metastatic and/or unresectable HCC). In some embodiments, the high-risk liver cancer comprises one or more of the following features: a VP4 PVTT, bile duct invasion, and/or tumor occupancy of 50% of the liver.

In some embodiments of any of the methods, uses, or pharmaceutical compositions for use described herein, the treatment results in a response in the subject or population of subjects after treatment. For example, in some embodiments, the treatment increases the subject's or population of subjects' likelihood of having an objective response, extends the subject's or population of subjects' PFS, extends the subject's or population of subjects' OS, extends the subject's or population of subjects' time to radiographic progression (TTRP), extends the subject's or population of subjects' duration of response (DOR), and/or reduces the risk of death, for example, as compared to a reference treatment. In some embodiments, the treatment results in a median PFS of the population of subjects of at least about 5.6 months (e.g., between 5.6 months and 14 months (e.g., 5.6 months, 5.7 months, 5.8 months, 5.9 months, 6 months, 7 months, 8 months, 9 months, 10 months, 11 months, 12 months, 13 months, or 14 months)). In some embodiments, the treatment results in a median PFS of the population of subjects of at least about 6.83 months (e.g., 6.9 months, 7 months, 8 months, 9 months, 10 months, 11 months, 12 months, 13 months, or 14 months). In some embodiments, the treatment results in a median PFS of the population of subjects of at least about 5.6 months to at least about 6.83 months (e.g., at least about 5.7, 5.8, 5.9, 6.0, 6.1, 6.2, 6.3, 6.4, 6.5, 6.6, 6.7, 6.8, or 6.83 months). In some embodiments, the reference treatment comprises current standard of care. In some embodiments, the reference treatment comprises a PD-1 axis binding antagonist (e.g., atezolizumab) and a VEGF antagonist (e.g., bevacizumab) without an anti-TIGIT antagonist antibody. In some embodiments, the reference treatment comprises a PD-1 axis binding antagonist and a VEGF antagonist. In some embodiments, the reference treatment comprises a PD-1 axis binding antagonist and an anti-TIGIT antagonist antibody without a VEGF antagonist. In some embodiments, the reference treatment comprises a VEGF antagonist and an anti-TIGIT antagonist antibody without a PD-1 axis binding antagonist. In some embodiments, the reference treatment comprises a PD-1 axis binding antagonist without a VEGF antagonist and an anti-TIGIT antagonist antibody. In some embodiments, the reference treatment comprises a VEGF antagonist without an anti-TIGIT antagonist antibody and a PD-1 axis binding antagonist. In some embodiments, the reference treatment comprises an anti-TIGIT antagonist antibody without a VEGF antagonist and a PD-1 axis binding antagonist.

In some embodiments of any of the methods, uses, or pharmaceutical compositions for use described herein, the treatment response(s) may be improved as compared to any suitable standard of care cancer therapy. In some embodiments, standard of care cancer therapy is a standard of care liver cancer (e.g., HCC, including locally advanced or metastatic and/or unresectable HCC) therapy. In some embodiments, the standard of care liver cancer (e.g., HCC) therapy comprises a tyrosine kinase inhibitor. In some embodiments, the tyrosine kinase inhibitor is a multikinase inhibitor. In some embodiments, the multikinase inhibitor is sorafenib or lenvatinib. In some embodiments, the multikinase inhibitor is sorafenib.

In some embodiments of any of the methods, uses, or pharmaceutical compositions for use described herein, the anti-TIGIT antagonist antibody (e.g., an anti-TIGIT antagonist antibody as disclosed herein, e.g., tiragolumab), PD-1 axis binding antagonist (e.g., anti-PD-L1 antagonist antibody (e.g., atezolizumab)), VEGF antagonist (e.g., an anti-VEGF antibody (e.g., bevacizumab)), or a medicament thereof, may be administered, used in the manufacture of a medicament, or formulated for administration in conjunction with (either separately or together), one or more additional anti-cancer therapeutic agent(s) (e.g., a chemotherapeutic agent, a cytotoxic agent, a growth inhibitory agent, a radiotherapy/radiation therapy, and/or an anti-hormonal agent, such as those recited herein above).

In some embodiments of any of the methods, uses, or pharmaceutical compositions for use described herein, the subject or population of subjects is previously untreated for the liver cancer (e.g., HCC, including locally advanced or metastatic and/or unresectable HCC). In some instances, the subject or population of subjects has not received prior therapy. In some instances, the subject or population of subjects has received at least one line of prior therapy. In some instances, the subject or population of subjects has received two lines of prior therapy. In some instances, the subject or population of subjects has received at least one but no more than two prior systemic therapies and/or for whom no acceptable standard of care exists. In some instances, the subject or population of subjects has not received more than two lines of prior therapy. In some instances, the prior therapy is chemotherapy, surgery, and/or radiotherapy. In some instances, the prior therapy is an immunotherapy. In some embodiments, the subject or population of subjects is previously untreated for liver cancer (e.g., HCC (e.g., locally advanced or metastatic and/or unresectable HCC)). In some embodiments, the subject or population of subjects is previously untreated for HCC. In some embodiments, the subject or population of subjects is previously untreated for locally advanced or metastatic and/or unresectable HCC.

In some embodiments of any of the methods, uses, or pharmaceutical compositions for use described herein, the subject or population of subjects has received no prior systemic therapy for liver cancer (e.g., HCC, including locally advanced or metastatic and/or unresectable HCC). In some embodiments, the subject or population of subjects has received no prior systemic therapy for liver cancer (e.g., HCC).

In some embodiments of any of the methods, uses, or pharmaceutical compositions for use described herein, the subject or population of subjects has not received prior treatment with a PD-1 axis binding antagonist, VEGF antagonist, or anti-TIGIT antagonist antibody.

In some embodiments of any of the methods, uses, or pharmaceutical compositions for use described herein, the subject or population of subjects may have any suitable Child-Pugh liver function. For example, in some embodiments, the subject or population of subjects has Child-Pugh class A liver function.

Any suitable PD-1 axis binding antagonist, VEGF antagonist, or anti-TIGIT antagonist antibody may be used in the methods, uses, or pharmaceutical compositions for use described herein. For example, any of the PD-1 axis binding antagonists, VEGF antagonists, or anti-TIGIT antagonist antibodies known in the art or described herein may be used in the methods, uses, or pharmaceutical compositions for use described herein. In some embodiments, the PD-1 axis binding antagonist is an anti-PD-L1 antibody or an anti-PD-1 antibody. In some embodiments, the anti-PD-L1 antibody is atezolizumab. In some embodiments, the VEGF antagonist is an anti-VEGF antibody. In some embodiments, the anti-VEGF antibody is bevacizumab. In some embodiments, the anti-TIGIT antagonist antibody is tiragolumab.

Exemplary Methods

In one aspect, the invention provides a method of treating a subject or population of subjects having a HCC (e.g., locally advanced or metastatic and/or unresectable HCC) by administering to the subject or population of subjects one or more dosing cycles of an anti-TIGIT antagonist antibody at a dose (e.g., a fixed dose) of 600 mg every three weeks and atezolizumab at a dose (e.g., a fixed dose) of 1200 mg every three weeks, wherein the anti-TIGIT antagonist antibody has a VH domain having the amino acid sequence of SEQ ID NO: 17 or 18 and a VL domain having the amino acid sequence of SEQ ID NO: 19, as described in further detail below. In some instances, the anti-TIGIT antagonist antibody has a VH domain having the amino acid sequence of SEQ ID NO: 17 and a VL domain having the amino acid sequence of SEQ ID NO: 19. In some instances, the anti-TIGIT antagonist antibody has a VH domain having the amino acid sequence of SEQ ID NO: 18 and a VL domain having the amino acid sequence of SEQ ID NO: 19. In some instances, the subject or population of subjects is previously untreated for HCC, e.g., locally advanced or metastatic and/or unresectable HCC.

In another aspect, the invention provides a method of treating a subject or population of subjects having a HCC (e.g., locally advanced or metastatic and/or unresectable HCC) by administering to the subject or population of subjects one or more dosing cycles of an anti-TIGIT antagonist antibody at a dose (e.g., a fixed dose) of 600 mg every three weeks, atezolizumab at a dose (e.g., a fixed dose) of 1200 mg every three weeks, and bevacizumab at a dose of 15 mg/kg every three weeks, wherein the anti-TIGIT antagonist antibody has a VH domain having the amino acid sequence of SEQ ID NO: 17 or 18 and a VL domain having the amino acid sequence of SEQ ID NO: 19, as described in further detail below. In some instances, the anti-TIGIT antagonist antibody has a VH domain having the amino acid sequence of SEQ ID NO: 17 and a VL domain having the amino acid sequence of SEQ ID NO: 19. In some instances, the anti-TIGIT antagonist antibody has a VH domain having the amino acid sequence of SEQ ID NO: 18 and a VL domain having the amino acid sequence of SEQ ID NO: 19.

In another aspect, the invention provides a method of treating a subject or population of subjects having a HCC (e.g., locally advanced or metastatic and/or unresectable HCC) by administering to the subject or population of subjects one or more dosing cycles of tiragolumab at a dose (e.g., a fixed dose) of 600 mg every three weeks and atezolizumab at a dose (e.g., a fixed dose) of 1200 mg every three weeks.

In another aspect, the invention provides a method of treating a subject or population of subjects having a HCC (e.g., locally advanced or metastatic and/or unresectable HCC) by administering to the subject or population of subjects one or more dosing cycles of tiragolumab at a dose (e.g., a fixed dose) of 600 mg every three weeks, atezolizumab at a dose (e.g., a fixed dose) of 1200 mg every three weeks, and bevacizumab at a dose of 15 mg/kg every three weeks.

In another aspect, the invention provides a method of treating a subject or population of subjects having a HCC (e.g., locally advanced or metastatic and/or unresectable HCC) by administering to the subject or population of subjects one or more dosing cycles of tiragolumab at a dose (e.g., a fixed dose) of 420 mg every two weeks and atezolizumab at a dose (e.g., a fixed dose) of 840 mg every two weeks.

In another aspect, the invention provides a method of treating a subject or population of subjects having a HCC (e.g., locally advanced or metastatic and/or unresectable HCC) by administering to the subject or population of subjects one or more dosing cycles of tiragolumab at a dose (e.g., a fixed dose) of 420 mg every two weeks, atezolizumab at a dose (e.g., a fixed dose) of 840 mg every two weeks, and bevacizumab at a dose of 5 mg/kg, 7.5 mg/kg, or 10 mg/kg every two weeks.

In another aspect, the invention provides a method of treating a subject or population of subjects having a HCC (e.g., locally advanced or metastatic and/or unresectable HCC) by administering to the subject or population of subjects one or more dosing cycles of tiragolumab at a dose (e.g., a fixed dose) of 840 mg every four weeks and atezolizumab at a dose (e.g., a fixed dose) of 1680 mg every four weeks.

In another aspect, the invention provides a method of treating a subject or population of subjects having a HCC (e.g., locally advanced or metastatic and/or unresectable HCC) by administering to the subject or population of subjects one or more dosing cycles of tiragolumab at a dose (e.g., a fixed dose) of 840 mg every four weeks, atezolizumab at a dose (e.g., a fixed dose) of 1680 mg every four weeks, and bevacizumab at a dose of 5 mg/kg, 7.5 mg/kg, or 10 mg/kg every two weeks.

In another aspect, the invention provides an anti-TIGIT antagonist antibody (e.g., an anti-TIGIT antagonist antibody disclosed herein, e.g., tiragolumab) and an PD-1 axis binding antagonist (e.g., an anti-PD-L1 antagonist antibody (e.g., atezolizumab)), for use in a method of treating a subject or population of subjects having a liver cancer (e.g., hepatocellular carcinoma (HCC), including locally advanced or metastatic and/or unresectable HCC), wherein the method comprises administering to the subject or population of subjects one or more dosing cycles of an effective amount of an anti-TIGIT antagonist antibody and an effective amount of a PD-1 axis binding antagonist (e.g., an anti-PD-L1 antagonist antibody (e.g., atezolizumab)).

In another aspect, the invention provides an anti-TIGIT antagonist antibody (e.g., an anti-TIGIT antagonist antibody disclosed herein, e.g., tiragolumab), an PD-1 axis binding antagonist (e.g., an anti-PD-L1 antagonist antibody (e.g., atezolizumab)), and a VEGF antagonist (e.g., an anti-VEGF antibody (e.g., bevacizumab)) for use in a method of treating a subject or population of subjects having a liver cancer (e.g., hepatocellular carcinoma (HCC), including locally advanced or metastatic and/or unresectable HCC), wherein the method comprises administering to the subject or population of subjects one or more dosing cycles of an effective amount of an anti-TIGIT antagonist antibody, an effective amount of a PD-1 axis binding antagonist (e.g., an anti-PD-L1 antagonist antibody (e.g., atezolizumab)), and an effective amount of a VEGF antagonist (e.g., an anti-VEGF antibody (e.g., bevacizumab)).

Exemplary Medicaments and Uses Thereof

In another aspect, the invention provides uses of an anti-TIGIT antagonist antibody (e.g., an anti-TIGIT antagonist antibody disclosed herein, e.g., tiragolumab) and a PD-1 axis binding antagonist (e.g., an anti-PD-L1 antagonist antibody (e.g., atezolizumab)) in the manufacture or preparation of a medicament for use in a method of treating a subject or population of subjects having a liver cancer (e.g., hepatocellular carcinoma (HCC), including locally advanced or metastatic and/or unresectable HCC), wherein the method comprises administering to the subject or population of subjects one or more dosing cycles of the medicament, and wherein the medicament is formulated for administration of the anti-TIGIT antagonist antibody at a dose (e.g., a fixed dose) of between about 30 mg to about 1200 mg every three weeks and the PD-1 axis binding antagonist (e.g., an anti-PD-L1 antagonist antibody (e.g., atezolizumab)) at a dose (e.g., a fixed dose) of between about 80 mg to about 2000 mg every three weeks. In some aspects, the medicament is formulated for administration of the anti-TIGIT antagonist antibody at a dose (e.g., a fixed dose) of between 30 mg to 1200 mg every three weeks and the PD-1 axis binding antagonist (e.g., an anti-PD-L1 antagonist antibody (e.g., atezolizumab)) at a dose (e.g., a fixed dose) of between 80 mg to 2000 mg every three weeks.

In another aspect, the invention provides uses of an anti-TIGIT antagonist antibody (e.g., an anti-TIGIT antagonist antibody disclosed herein, e.g., tiragolumab), a PD-1 axis binding antagonist (e.g., an anti-PD-L1 antagonist antibody (e.g., atezolizumab)), and a VEGF antagonist (e.g., an anti-VEGF antibody (e.g., bevacizumab)) in the manufacture or preparation of a medicament for use in a method of treating a subject or population of subjects having a liver cancer (e.g., hepatocellular carcinoma (HCC), including locally advanced or metastatic and/or unresectable HCC), wherein the method comprises administering to the subject or population of subjects one or more dosing cycles of the medicament, and wherein the medicament is formulated for administration of the anti-TIGIT antagonist antibody at a dose (e.g., a fixed dose) of between about 30 mg to about 1200 mg every three weeks, the PD-1 axis binding antagonist (e.g., an anti-PD-L1 antagonist antibody (e.g., atezolizumab)) at a dose (e.g., a fixed dose) of between about 80 mg to about 2000 mg every three weeks, and the VEGF antagonist (e.g., an anti-VEGF antibody (e.g., bevacizumab)) at a dose of between about 0.1 mg/kg and 50 mg/kg every three weeks. In some aspects, the medicament is formulated for administration of the anti-TIGIT antagonist antibody at a dose (e.g., a fixed dose) of between 30 mg to 1200 mg every three weeks, the PD-1 axis binding antagonist (e.g., an anti-PD-L1 antagonist antibody (e.g., atezolizumab)) at a dose (e.g., a fixed dose) of between 80 mg to 2000 mg every three weeks, and the VEGF antagonist (e.g., an anti-VEGF antibody (e.g., bevacizumab)) at a dose of between about 0.1 mg/kg and 50 mg/kg every three weeks.

In another aspect, the invention provides uses of an anti-TIGIT antagonist antibody (e.g., an anti-TIGIT antagonist antibody disclosed herein, e.g., tiragolumab) and a PD-1 axis binding antagonist (e.g., an anti-PD-L1 antagonist antibody (e.g., atezolizumab)) in the manufacture or preparation of a medicament for use in a method of treating a subject or population of subjects having a liver cancer (e.g., hepatocellular carcinoma (HCC), including locally advanced or metastatic and/or unresectable HCC), wherein the method comprises administering to the subject or population of subjects one or more dosing cycles of the medicament, and wherein the medicament is formulated for administration of the anti-TIGIT antagonist antibody at a dose (e.g., a fixed dose) of between about 30 mg to about 600 mg every two weeks and the PD-1 axis binding antagonist (e.g., an anti-PD-L1 antagonist antibody (e.g., atezolizumab)) at a dose (e.g., a fixed dose) of between about 80 mg to about 1200 mg every two weeks. In some aspects, the medicament is formulated for administration of the anti-TIGIT antagonist antibody at a dose (e.g., a fixed dose) of between 30 mg to 600 mg every two weeks and the PD-1 axis binding antagonist (e.g., an anti-PD-L1 antagonist antibody (e.g., atezolizumab)) at a dose (e.g., a fixed dose) of between 80 mg to 1200 mg every two weeks.

In another aspect, the invention provides uses of an anti-TIGIT antagonist antibody (e.g., an anti-TIGIT antagonist antibody disclosed herein, e.g., tiragolumab), a PD-1 axis binding antagonist (e.g., an anti-PD-L1 antagonist antibody (e.g., atezolizumab)), and a VEGF antagonist (e.g., an anti-VEGF antibody (e.g., bevacizumab)) in the manufacture or preparation of a medicament for use in a method of treating a subject or population of subjects having a liver cancer (e.g., hepatocellular carcinoma (HCC), including locally advanced or metastatic and/or unresectable HCC), wherein the method comprises administering to the subject or population of subjects one or more dosing cycles of the medicament, and wherein the medicament is formulated for administration of the anti-TIGIT antagonist antibody at a dose (e.g., a fixed dose) of between about 30 mg to about 600 mg every two weeks, the PD-1 axis binding antagonist (e.g., an anti-PD-L1 antagonist antibody (e.g., atezolizumab)) at a dose (e.g., a fixed dose) of between about 80 mg to about 1200 mg every two weeks, and the VEGF antagonist (e.g., an anti-VEGF antibody (e.g., bevacizumab)) at a dose of between about 0.1 mg/kg and 50 mg/kg every two weeks. In some aspects, the medicament is formulated for administration of the anti-TIGIT antagonist antibody at a dose (e.g., a fixed dose) of between 30 mg to 600 mg every two weeks, the PD-1 axis binding antagonist (e.g., an anti-PD-L1 antagonist antibody (e.g., atezolizumab)) at a dose (e.g., a fixed dose) of between 80 mg to 1200 mg every two weeks, and the VEGF antagonist (e.g., an anti-VEGF antibody (e.g., bevacizumab)) at a dose of between 0.1 mg/kg and 50 mg/kg every two weeks.

In another aspect, the invention provides uses of an anti-TIGIT antagonist antibody (e.g., an anti-TIGIT antagonist antibody disclosed herein, e.g., tiragolumab) and an PD-1 axis binding antagonist (e.g., an anti-PD-L1 antagonist antibody (e.g., atezolizumab)) in the manufacture or preparation of a medicament for use in a method of treating a subject or population of subjects having a liver cancer (e.g., hepatocellular carcinoma (HCC), including locally advanced or metastatic and/or unresectable HCC), wherein the method comprises administering to the subject or population of subjects one or more dosing cycles of the medicament, and wherein the medicament is formulated for administration of the anti-TIGIT antagonist antibody at a dose (e.g., a fixed dose) of between about 600 mg to about 1200 mg every four weeks and the PD-1 axis binding antagonist (e.g., an anti-PD-L1 antagonist antibody (e.g., atezolizumab)) at a dose (e.g., a fixed dose) of between about 1200 mg to about 2000 mg every four weeks. In some aspects, the medicament is formulated for administration of the anti-TIGIT antagonist antibody at a dose (e.g., a fixed dose) of between 600 mg to 1200 mg every four weeks and the PD-1 axis binding antagonist (e.g., an anti-PD-L1 antagonist antibody (e.g., atezolizumab)) at a dose (e.g., a fixed dose) of between 1200 mg to 2000 mg every four weeks.

In another aspect, the invention provides uses of an anti-TIGIT antagonist antibody (e.g., an anti-TIGIT antagonist antibody disclosed herein, e.g., tiragolumab), a PD-1 axis binding antagonist (e.g., an anti-PD-L1 antagonist antibody (e.g., atezolizumab)), and a VEGF antagonist (e.g., an anti-VEGF antibody (e.g., bevacizumab)) in the manufacture or preparation of a medicament for use in a method of treating a subject or population of subjects having a liver cancer (e.g., hepatocellular carcinoma (HCC), including locally advanced or metastatic and/or unresectable HCC), wherein the method comprises administering to the subject or population of subjects one or more dosing cycles of the medicament, and wherein the medicament is formulated for administration of the anti-TIGIT antagonist antibody at a dose (e.g., a fixed dose) of between about 600 mg to about 1200 mg every four weeks, the PD-1 axis binding antagonist (e.g., an anti-PD-L1 antagonist antibody (e.g., atezolizumab)) at a dose (e.g., a fixed dose) of between about 1200 mg to about 2000 mg every four weeks, and the VEGF antagonist (e.g., an anti-VEGF antibody (e.g., bevacizumab)) at a dose of between about 0.1 mg/kg and 50 mg/kg every two weeks. In some aspects, the medicament is formulated for administration of the anti-TIGIT antagonist antibody at a dose (e.g., a fixed dose) of between 600 mg to 1200 mg every four weeks, the PD-1 axis binding antagonist (e.g., an anti-PD-L1 antagonist antibody (e.g., atezolizumab)) at a dose (e.g., a fixed dose) of between 1200 mg to 2000 mg every four weeks, and the VEGF antagonist (e.g., an anti-VEGF antibody (e.g., bevacizumab)) at a dose of between 0.1 mg/kg and 50 mg/kg every two weeks.

In another aspect, the invention provides uses of an anti-TIGIT antagonist antibody (e.g., an anti-TIGIT antagonist antibody disclosed herein, e.g., tiragolumab) and a PD-1 axis binding antagonist (e.g., an anti-PD-L1 antagonist antibody (e.g., atezolizumab)) in the manufacture of a medicament for use in a method of treating a subject or population of subjects having a liver cancer (e.g., hepatocellular carcinoma (HCC), including locally advanced or metastatic and/or unresectable HCC), wherein the method comprises administering to the subject or population of subjects one or more dosing cycles of the medicament and an anti-TIGIT antagonist antibody, and wherein the medicament is formulated for administration of the PD-1 axis binding antagonist (e.g., an anti-PD-L1 antagonist antibody (e.g., atezolizumab)) at a dose (e.g., a fixed dose) of between about 80 mg to about 2000 mg every three weeks and the anti-TIGIT antagonist antibody is to be administered at a dose (e.g., a fixed dose) of between about 30 mg to about 1200 mg every three weeks. In some aspects, the medicament is formulated for administration of the PD-1 axis binding antagonist (e.g., an anti-PD-L1 antagonist antibody (e.g., atezolizumab)) at a dose (e.g., a fixed dose) of between 80 mg to 2000 mg every three weeks and the anti-TIGIT antagonist antibody is to be administered at a dose (e.g., a fixed dose) of between 30 mg to 1200 mg every three weeks.

In another aspect, the invention provides uses of an anti-TIGIT antagonist antibody (e.g., an anti-TIGIT antagonist antibody disclosed herein, e.g., tiragolumab), a PD-1 axis binding antagonist (e.g., an anti-PD-L1 antagonist antibody (e.g., atezolizumab)), and a VEGF antagonist (e.g., an anti-VEGF antibody (e.g., bevacizumab)) in the manufacture of a medicament for use in a method of treating a subject or population of subjects having a liver cancer (e.g., hepatocellular carcinoma (HCC), including locally advanced or metastatic and/or unresectable HCC), wherein the method comprises administering to the subject or population of subjects one or more dosing cycles of the medicament and an anti-TIGIT antagonist antibody, and wherein the medicament is formulated for administration of the PD-1 axis binding antagonist (e.g., an anti-PD-L1 antagonist antibody (e.g., atezolizumab)) at a dose (e.g., a fixed dose) of between about 80 mg to about 2000 mg every three weeks, the anti-TIGIT antagonist antibody is to be administered at a dose (e.g., a fixed dose) dose of between about 30 mg to about 1200 mg every three weeks, and the VEGF antagonist (e.g., an anti-VEGF antibody (e.g., bevacizumab)) at a dose of between about 0.1 mg/kg and 50 mg/kg every three weeks. In some aspects, the medicament is formulated for administration of the PD-1 axis binding antagonist (e.g., an anti-PD-L1 antagonist antibody (e.g., atezolizumab)) at a dose (e.g., a fixed dose) of between 80 mg to 2000 mg every three weeks, the anti-TIGIT antagonist antibody is to be administered at a dose (e.g., a fixed dose) dose of between 30 mg to 1200 mg every three weeks, and the VEGF antagonist (e.g., an anti-VEGF antibody (e.g., bevacizumab)) at a dose of between 0.1 mg/kg and 50 mg/kg every three weeks.

In another aspect, the invention provides uses of an anti-TIGIT antagonist antibody (e.g., an anti-TIGIT antagonist antibody disclosed herein, e.g., tiragolumab) and a PD-1 axis binding antagonist (e.g., an anti-PD-L1 antagonist antibody (e.g., atezolizumab)) in the manufacture of a medicament for use in a method of treating a subject or population of subjects having a liver cancer (e.g., hepatocellular carcinoma (HCC), including locally advanced or metastatic and/or unresectable HCC), wherein the method comprises administering to the subject or population of subjects one or more dosing cycles of the medicament and an anti-TIGIT antagonist antibody, and wherein the medicament is formulated for administration of the PD-1 axis binding antagonist (e.g., an anti-PD-L1 antagonist antibody (e.g., atezolizumab)) at a dose (e.g., a fixed dose) of between about 80 mg to about 1200 mg every two weeks and the anti-TIGIT antagonist antibody is to be administered at a dose (e.g., a fixed dose) of between about 30 mg to about 600 mg every two weeks. In some aspects, the medicament is formulated for administration of the PD-1 axis binding antagonist (e.g., an anti-PD-L1 antagonist antibody (e.g., atezolizumab)) at a dose (e.g., a fixed dose) of between 80 mg to 1200 mg every two weeks and the anti-TIGIT antagonist antibody is to be administered at a dose (e.g., a fixed dose) of between 30 mg to 600 mg every two weeks.

In another aspect, the invention provides uses of an anti-TIGIT antagonist antibody (e.g., an anti-TIGIT antagonist antibody disclosed herein, e.g., tiragolumab), a PD-1 axis binding antagonist (e.g., an anti-PD-L1 antagonist antibody (e.g., atezolizumab)), and a VEGF antagonist (e.g., an anti-VEGF antibody (e.g., bevacizumab)) in the manufacture of a medicament for use in a method of treating a subject or population of subjects having a liver cancer (e.g., hepatocellular carcinoma (HCC), including locally advanced or metastatic and/or unresectable HCC), wherein the method comprises administering to the subject or population of subjects one or more dosing cycles of the medicament and an anti-TIGIT antagonist antibody, and wherein the medicament is formulated for administration of the PD-1 axis binding antagonist (e.g., an anti-PD-L1 antagonist antibody (e.g., atezolizumab)) at a dose (e.g., a fixed dose) of between about 80 mg to about 1200 mg every two weeks, the anti-TIGIT antagonist antibody is to be administered at a dose (e.g., a fixed dose) of between about 30 mg to about 600 mg every two weeks, and the VEGF antagonist (e.g., an anti-VEGF antibody (e.g., bevacizumab)) at a dose of between about 0.1 mg/kg and 50 mg/kg every two weeks. In some aspects, the medicament is formulated for administration of the PD-1 axis binding antagonist (e.g., an anti-PD-L1 antagonist antibody (e.g., atezolizumab)) at a dose (e.g., a fixed dose) of between 80 mg to 1200 mg every two weeks, the anti-TIGIT antagonist antibody is to be administered at a dose (e.g., a fixed dose) of between 30 mg to 600 mg every two weeks, and the VEGF antagonist (e.g., an anti-VEGF antibody (e.g., bevacizumab)) at a dose of between 0.1 mg/kg and 50 mg/kg every two weeks.

In another aspect, the invention provides uses of an anti-TIGIT antagonist antibody (e.g., an anti-TIGIT antagonist antibody disclosed herein, e.g., tiragolumab) and a PD-1 axis binding antagonist (e.g., an anti-PD-L1 antagonist antibody (e.g., atezolizumab)) in the manufacture of a medicament for use in a method of treating a subject or population of subjects having a liver cancer (e.g., hepatocellular carcinoma (HCC), including locally advanced or metastatic and/or unresectable HCC), wherein the method comprises administering to the subject or population of subjects one or more dosing cycles of the medicament and an anti-TIGIT antagonist antibody, and wherein the medicament is formulated for administration of the PD-1 axis binding antagonist (e.g., an anti-PD-L1 antagonist antibody (e.g., atezolizumab)) at a dose (e.g., a fixed dose) of between about 1200 mg to about 2000 mg every four weeks and the anti-TIGIT antagonist antibody is to be administered at a dose (e.g., a fixed dose) of between about 600 mg to about 1200 mg every four weeks. In some aspects, the medicament is formulated for administration of the PD-1 axis binding antagonist (e.g., an anti-PD-L1 antagonist antibody (e.g., atezolizumab)) at a dose (e.g., a fixed dose) of between 1200 mg to 2000 mg every four weeks and the anti-TIGIT antagonist antibody is to be administered at a dose (e.g., a fixed dose) of between 600 mg to 1200 mg every four weeks.

In another aspect, the invention provides uses of an anti-TIGIT antagonist antibody (e.g., an anti-TIGIT antagonist antibody disclosed herein, e.g., tiragolumab), a PD-1 axis binding antagonist (e.g., an anti-PD-L1 antagonist antibody (e.g., atezolizumab)), and a VEGF antagonist (e.g., an anti-VEGF antibody (e.g., bevacizumab)) in the manufacture of a medicament for use in a method of treating a subject or population of subjects having a liver cancer (e.g., hepatocellular carcinoma (HCC), including locally advanced or metastatic and/or unresectable HCC), wherein the method comprises administering to the subject or population of subjects one or more dosing cycles of the medicament and an anti-TIGIT antagonist antibody, and wherein the medicament is formulated for administration of the PD-1 axis binding antagonist (e.g., an anti-PD-L1 antagonist antibody (e.g., atezolizumab)) at a dose (e.g., a fixed dose) of between about 1200 mg to about 2000 mg every four weeks, the anti-TIGIT antagonist antibody is to be administered at a dose (e.g., a fixed dose) of between about 600 mg to about 1200 mg every four weeks, and the VEGF antagonist (e.g., an anti-VEGF antibody (e.g., bevacizumab)) at a dose of between about 0.1 mg/kg and 50 mg/kg every two weeks. In some aspects, the medicament is formulated for administration of the PD-1 axis binding antagonist (e.g., an anti-PD-L1 antagonist antibody (e.g., atezolizumab)) at a dose (e.g., a fixed dose) of between 1200 mg to 2000 mg every four weeks, the anti-TIGIT antagonist antibody is to be administered at a dose (e.g., a fixed dose) of between 600 mg to 1200 mg every four weeks, and the VEGF antagonist (e.g., an anti-VEGF antibody (e.g., bevacizumab)) at a dose of between 0.1 mg/kg and 50 mg/kg every two weeks.

In another aspect, the invention provides uses of an anti-TIGIT antagonist antibody and atezolizumab in the manufacture of a medicament for use in a method of treating a subject or population of subjects having a liver cancer (e.g., hepatocellular carcinoma (HCC), including locally advanced or metastatic and/or unresectable HCC), wherein the method comprises administering to the subject or population of subjects one or more dosing cycles of the medicament, wherein the medicament is formulated for administration of the anti-TIGIT antagonist antibody at a dose (e.g., a fixed dose) of 600 mg every three weeks and atezolizumab at a dose (e.g., a fixed dose) of 1200 mg every three weeks and wherein the anti-TIGIT antagonist antibody comprises: a VH domain comprising the amino acid sequence of SEQ ID NO: 17 or 18 and a VL domain comprising the amino acid sequence of SEQ ID NO: 19, as described in further detail below. In some instances, the anti-TIGIT antagonist antibody has a VH domain having the amino acid sequence of SEQ ID NO: 17 and a VL domain having the amino acid sequence of SEQ ID NO: 19. In some instances, the anti-TIGIT antagonist antibody has a VH domain having the amino acid sequence of SEQ ID NO: 18 and a VL domain having the amino acid sequence of SEQ ID NO: 19.

In another aspect, the invention provides uses of an anti-TIGIT antagonist antibody, atezolizumab, and bevacizumab in the manufacture of a medicament for use in a method of treating a subject or population of subjects having a liver cancer (e.g., hepatocellular carcinoma (HCC), including locally advanced or metastatic and/or unresectable HCC), wherein the method comprises administering to the subject or population of subjects one or more dosing cycles of the medicament, wherein the medicament is formulated for administration of the anti-TIGIT antagonist antibody at a dose (e.g., a fixed dose) of 600 mg every three weeks, atezolizumab at a dose (e.g., a fixed dose) of 1200 mg every three weeks, bevacizumab at a dose of 15 mg/kg every three weeks and wherein the anti-TIGIT antagonist antibody comprises: a VH domain comprising the amino acid sequence of SEQ ID NO: 17 or 18 and a VL domain comprising the amino acid sequence of SEQ ID NO: 19, as described in further detail below. In some instances, the anti-TIGIT antagonist antibody has a VH domain having the amino acid sequence of SEQ ID NO: 17 and a VL domain having the amino acid sequence of SEQ ID NO: 19. In some instances, the anti-TIGIT antagonist antibody has a VH domain having the amino acid sequence of SEQ ID NO: 18 and a VL domain having the amino acid sequence of SEQ ID NO: 19.

In another aspect, the invention provides uses of an anti-TIGIT antagonist antibody and atezolizumab in the manufacture of a medicament for use in a method of treating a subject or population of subjects having a liver cancer (e.g., hepatocellular carcinoma (HCC), including locally advanced or metastatic and/or unresectable HCC), wherein the method comprises administering to the subject or population of subjects one or more dosing cycles of the medicament, wherein the medicament is formulated for administration of the anti-TIGIT antagonist antibody at a dose (e.g., a fixed dose) of 420 mg every two weeks and atezolizumab at a dose (e.g., a fixed dose) of 840 mg every two weeks, and wherein the anti-TIGIT antagonist antibody comprises: a VH domain comprising the amino acid sequence of SEQ ID NO: 17 or 18 and a VL domain comprising the amino acid sequence of SEQ ID NO: 19, as described in further detail below. In some instances, the anti-TIGIT antagonist antibody has a VH domain having the amino acid sequence of SEQ ID NO: 17 and a VL domain having the amino acid sequence of SEQ ID NO: 19. In some instances, the anti-TIGIT antagonist antibody has a VH domain having the amino acid sequence of SEQ ID NO: 18 and a VL domain having the amino acid sequence of SEQ ID NO: 19.

In another aspect, the invention provides uses of an anti-TIGIT antagonist antibody, atezolizumab, and bevacizumab in the manufacture of a medicament for use in a method of treating a subject or population of subjects having a liver cancer (e.g., hepatocellular carcinoma (HCC), including locally advanced or metastatic and/or unresectable HCC), wherein the method comprises administering to the subject or population of subjects one or more dosing cycles of the medicament, wherein the medicament is formulated for administration of the anti-TIGIT antagonist antibody at a dose (e.g., a fixed dose) of 420 mg every two weeks, atezolizumab at a dose (e.g., a fixed dose) of 840 mg every two weeks, and bevacizumab at a dose of 5 mg/kg, 7.5. mg/kg, or 10 mg/kg every two weeks and wherein the anti-TIGIT antagonist antibody comprises: a VH domain comprising the amino acid sequence of SEQ ID NO: 17 or 18 and a VL domain comprising the amino acid sequence of SEQ ID NO: 19, as described in further detail below. In some instances, the anti-TIGIT antagonist antibody has a VH domain having the amino acid sequence of SEQ ID NO: 17 and a VL domain having the amino acid sequence of SEQ ID NO: 19. In some instances, the anti-TIGIT antagonist antibody has a VH domain having the amino acid sequence of SEQ ID NO: 18 and a VL domain having the amino acid sequence of SEQ ID NO: 19.

In another aspect, the invention provides uses of an anti-TIGIT antagonist antibody and atezolizumab in the manufacture of a medicament for use in a method of treating a subject or population of subjects having a liver cancer (e.g., hepatocellular carcinoma (HCC), including locally advanced or metastatic and/or unresectable HCC), wherein the method comprises administering to the subject or population of subjects one or more dosing cycles of the medicament, wherein the medicament is formulated for administration of the anti-TIGIT antagonist antibody at a dose (e.g., a fixed dose) of 840 mg every four weeks and atezolizumab at a dose (e.g., a fixed dose) of 1680 mg every four weeks, and wherein the anti-TIGIT antagonist antibody comprises: a VH domain comprising the amino acid sequence of SEQ ID NO: 17 or 18 and a VL domain comprising the amino acid sequence of SEQ ID NO: 19, as described in further detail below. In some instances, the anti-TIGIT antagonist antibody has a VH domain having the amino acid sequence of SEQ ID NO: 17 and a VL domain having the amino acid sequence of SEQ ID NO: 19. In some instances, the anti-TIGIT antagonist antibody has a VH domain having the amino acid sequence of SEQ ID NO: 18 and a VL domain having the amino acid sequence of SEQ ID NO: 19.

In another aspect, the invention provides uses of an anti-TIGIT antagonist antibody, atezolizumab, and bevacizumab in the manufacture of a medicament for use in a method of treating a subject or population of subjects having a liver cancer (e.g., hepatocellular carcinoma (HCC), including locally advanced or metastatic and/or unresectable HCC), wherein the method comprises administering to the subject or population of subjects one or more dosing cycles of the medicament, wherein the medicament is formulated for administration of the anti-TIGIT antagonist antibody at a dose (e.g., a fixed dose) of 840 mg every four weeks, atezolizumab at a dose (e.g., a fixed dose) of 1680 mg every four weeks, bevacizumab at a dose of 5 mg/kg, 7.5 mg/kg, or 10 mg/kg every two weeks and wherein the anti-TIGIT antagonist antibody comprises: a VH domain comprising the amino acid sequence of SEQ ID NO: 17 or 18 and a VL domain comprising the amino acid sequence of SEQ ID NO: 19, as described in further detail below. In some instances, the anti-TIGIT antagonist antibody has a VH domain having the amino acid sequence of SEQ ID NO: 17 and a VL domain having the amino acid sequence of SEQ ID NO: 19. In some instances, the anti-TIGIT antagonist antibody has a VH domain having the amino acid sequence of SEQ ID NO: 18 and a VL domain having the amino acid sequence of SEQ ID NO: 19.

Dosing Regimens and Administration

The therapeutic methods and uses of the invention described herein include, in one aspect, administering to a subject or population of subjects having a liver cancer (e.g., hepatocellular carcinoma (HCC), including locally advanced or metastatic and/or unresectable HCC) a treatment regimen comprising an effective amount of a PD-1 axis binding antagonist (e.g., an anti-PD-L1 antagonist antibody (e.g., atezolizumab)), a VEGF antagonist (e.g., an anti-VEGF antibody (e.g., bevacizumab)), and an anti-TIGIT antagonist antibody (e.g., tiragolumab).

The pharmaceutical compositions described herein can be formulated for administration as described below and in Section III(K).

Effective Dosages

In some instances, the anti-TIGIT antagonist antibody (e.g., an anti-TIGIT antagonist antibody as disclosed herein, e.g., tiragolumab) is administered in a combination therapy (e.g., a combination treatment with a PD-1 axis binding antagonist (e.g., an anti-PD-L1 antagonist antibody (e.g., atezolizumab)) with a VEGF antagonist (e.g., an anti-VEGF antibody (e.g., bevacizumab)) at a dose as described in Section III(K), and the dose may be reduced as compared to a standard dose of the anti-TIGIT antagonist antibody administered as a monotherapy. Dosing of anti-TIGIT antagonist antibodies, PD-1 axis binding antagonists, and VEGF antagonists is described in Section III(K).

Dosing Cycles

In any of the methods and uses of the invention, the anti-TIGIT antagonist antibody (e.g., an anti-TIGIT antagonist antibody as disclosed herein, e.g., tiragolumab), the PD-1 axis binding antagonist (e.g., anti-PD-L1 antagonist antibody (e.g., atezolizumab)), or the VEGF antagonist (e.g., an anti-VEGF antibody (e.g., bevacizumab)) may be administered in one or more dosing cycles (e.g., 1, 2, 3, 4, 5, 6, 7, 8,9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 or more dosing cycles). In some instances, the dosing cycles of the anti-TIGIT antagonist antibody (e.g., an anti-TIGIT antagonist antibody as disclosed herein, e.g., tiragolumab), the PD-1 axis binding antagonist (e.g., anti-PD-L1 antagonist antibody (e.g., atezolizumab)), and/or the VEGF antagonist (e.g., an anti-VEGF antibody (e.g., bevacizumab)), continue until there is a loss of clinical benefit (e.g., confirmed disease progression, drug resistance, death, or unacceptable toxicity). In some instances, the length of each dosing cycle is about 14 to 28 days (e.g., 14 days, 15 days, 16 days, 17 days, 18 days, 19 days, 20 days, 21 days, 22 days, 23 days, 24 days, 25 days, 26 days, 27 days, or 28 days). In some instances, the length of each dosing cycle is about 21 days. In some instances, the length of each dosing cycle is about 14 days. In some instances, the length of each dosing cycle is about 28 days. In some instances, the anti-TIGIT antagonist antibody (e.g., an anti-TIGIT antagonist antibody as disclosed herein, e.g., tiragolumab) is administered on about Day 1 (e.g., Day 1±3 days) of each dosing cycle. For example, the anti-TIGIT antagonist antibody (e.g., an anti-TIGIT antagonist antibody as disclosed herein, e.g., tiragolumab) is administered intravenously at a dose (e.g., a fixed dose) of about 600 mg on Day 1 of each 21-day cycle (i.e., at a dose of about 600 mg every three weeks). In some instances, the PD-1 axis binding antagonist (e.g., an anti-PD-L1 antagonist antibody (e.g., atezolizumab)) is administered on about Day 1 (e.g., Day 1±3 days) of each dosing cycle. For example, the PD-1 axis binding antagonist (e.g., anti-PD-L1 antagonist antibody (e.g., atezolizumab)) is administered intravenously at a dose of about 1200 mg on Day 1 of each 21-day cycle (i.e., at a dose of about 1200 mg every three weeks). In some instances, the VEGF antagonist (e.g., an anti-VEGF antibody (e.g., bevacizumab)) is administered on about Day 1 (e.g., Day 1±3 days) of each dosing cycle. For example, the VEGF antagonist (e.g., an anti-VEGF antibody (e.g., bevacizumab)) is administered intravenously at a dose of about 15 mg/kg on Day 1 of each 21-day cycle (i.e., at a dose of about 15 mg/kg every three weeks). In some examples, For example, the anti-TIGIT antagonist antibody (e.g., an anti-TIGIT antagonist antibody as disclosed herein, e.g., tiragolumab) is administered intravenously at a dose (e.g., a fixed dose) of 600 mg on Day 1 of each 21-day cycle (i.e., at a dose of 600 mg every three weeks). In some instances, the PD-1 axis binding antagonist (e.g., an anti-PD-L1 antagonist antibody (e.g., atezolizumab)) is administered on Day 1 (e.g., Day 1±3 days) of each dosing cycle. For example, the PD-1 axis binding antagonist (e.g., anti-PD-L1 antagonist antibody (e.g., atezolizumab)) is administered intravenously at a dose of 1200 mg on Day 1 of each 21-day cycle (i.e., at a dose of 1200 mg every three weeks). In some instances, the VEGF antagonist (e.g., an anti-VEGF antibody (e.g., bevacizumab)) is administered on Day 1 (e.g., Day 1±3 days) of each dosing cycle. For example, the VEGF antagonist (e.g., an anti-VEGF antibody (e.g., bevacizumab)) is administered intravenously at a dose of 15 mg/kg on Day 1 of each 21-day cycle (i.e., at a dose of 15 mg/kg every three weeks).

In some instances, the anti-TIGIT antagonist antibody (e.g., an anti-TIGIT antagonist antibody as disclosed herein, e.g., tiragolumab), the PD-1 axis binding antagonist (e.g., anti-PD-L1 antagonist antibody (e.g., atezolizumab)), and the VEGF antagonist (e.g., an anti-VEGF antibody (e.g., bevacizumab)) are administered on about Day 1 (e.g., Day 1±3 days) of each dosing cycle.

In some instances, the anti-TIGIT antagonist antibody (e.g., an anti-TIGIT antagonist antibody as disclosed herein, e.g., tiragolumab) is administered intravenously at a dose of about 600 mg on Day 1 of each 21-day cycle (i.e., at a dose of about 600 mg every three weeks), the PD-1 axis binding antagonist (e.g., anti-PD-L1 antagonist antibody (e.g., atezolizumab)) is administered intravenously at a dose of about 1200 mg on Day 1 of each 21-day cycle (i.e., at a dose of about 1200 mg every three weeks), and the VEGF antagonist (e.g., an anti-VEGF antibody (e.g., bevacizumab)) is administered intravenously at a dose of about 15 mg/kg on Day 1 of each 21-day cycle (i.e., at a dose of about 15 mg/kg every three weeks). In some instances, the anti-TIGIT antagonist antibody (e.g., an anti-TIGIT antagonist antibody as disclosed herein, e.g., tiragolumab) is administered intravenously at a dose of 600 mg on Day 1 of each 21-day cycle (i.e., at a dose of 600 mg every three weeks), the PD-1 axis binding antagonist (e.g., anti-PD-L1 antagonist antibody (e.g., atezolizumab)) is administered intravenously at a dose of 1200 mg on Day 1 of each 21-day cycle (i.e., at a dose of 1200 mg every three weeks), and the VEGF antagonist (e.g., an anti-VEGF antibody (e.g., bevacizumab)) is administered intravenously at a dose of 15 mg/kg on Day 1 of each 21-day cycle (i.e., at a dose of 15 mg/kg every three weeks).

In other instances, the anti-TIGIT antagonist antibody (e.g., an anti-TIGIT antagonist antibody as disclosed herein, e.g., tiragolumab) is administered intravenously at a dose of about 420 mg on Day 1 of each 14-day cycle (i.e., at a dose of about 420 mg every two weeks), the PD-1 axis binding antagonist (e.g., anti-PD-L1 antagonist antibody (e.g., atezolizumab)) is administered intravenously at a dose of about 840 mg on Day 1 of each 14-day cycle (i.e., at a dose of about 840 mg every two weeks), and the VEGF antagonist (e.g., an anti-VEGF antibody (e.g., bevacizumab)) is administered intravenously at a dose of about 5 mg/kg, 7.5 mg/kg, or 10 mg/kg on Day 1 of each 14-day cycle (i.e., at a dose of about 5 mg/kg, 7.5 mg/kg, or 10 mg/kg every two weeks). In other instances, the anti-TIGIT antagonist antibody (e.g., an anti-TIGIT antagonist antibody as disclosed herein, e.g., tiragolumab) is administered intravenously at a dose of 420 mg on Day 1 of each 14-day cycle (i.e., at a dose of 420 mg every two weeks), the PD-1 axis binding antagonist (e.g., anti-PD-L1 antagonist antibody (e.g., atezolizumab)) is administered intravenously at a dose of 840 mg on Day 1 of each 14-day cycle (i.e., at a dose of 840 mg every two weeks), and the VEGF antagonist (e.g., an anti-VEGF antibody (e.g., bevacizumab)) is administered intravenously at a dose of 5 mg/kg, 7.5 mg/kg, or 10 mg/kg on Day 1 of each 14-day cycle (i.e., at a dose of 5 mg/kg, 7.5 mg/kg, or 10 mg/kg every two weeks).

In other instances, the anti-TIGIT antagonist antibody (e.g., an anti-TIGIT antagonist antibody as disclosed herein, e.g., tiragolumab) is administered intravenously at a dose of about 840 mg on Day 1 of each 28-day cycle (i.e., at a dose of about 840 mg every four weeks), the PD-1 axis binding antagonist (e.g., anti-PD-L1 antagonist antibody (e.g., atezolizumab)) is administered intravenously at a dose of about 1680 mg on Day 1 of each 28-day cycle (i.e., at a dose of about 1680 mg every four weeks), and the VEGF antagonist (e.g., an anti-VEGF antibody (e.g., bevacizumab)) is administered intravenously at a dose of about 5 mg/kg, 7.5 mg/kg, or 10 mg/kg on Day 1 and Day 15 of each 28-day cycle (i.e., at a dose of about 5 mg/kg, 7.5 mg/kg, or 10 mg/kg every two weeks). In other instances, the anti-TIGIT antagonist antibody (e.g., an anti-TIGIT antagonist antibody as disclosed herein, e.g., tiragolumab) is administered intravenously at a dose of 840 mg on Day 1 of each 28-day cycle (i.e., at a dose of 840 mg every four weeks), the PD-1 axis binding antagonist (e.g., anti-PD-L1 antagonist antibody (e.g., atezolizumab)) is administered intravenously at a dose of 1680 mg on Day 1 of each 28-day cycle (i.e., at a dose of 1680 mg every four weeks), and the VEGF antagonist (e.g., an anti-VEGF antibody (e.g., bevacizumab)) is administered intravenously at a dose of 5 mg/kg, 7.5 mg/kg, or 10 mg/kg on Day 1 and Day 15 of each 28-day cycle (i.e., at a dose of 5 mg/kg, 7.5 mg/kg, or 10 mg/kg every two weeks).

Administration Order and Observation Periods

In some instances, the PD-1 axis binding antagonist (e.g., anti-PD-L1 antagonist antibody (e.g., atezolizumab)) is administered to the subject or population of subjects before the VEGF antagonist (e.g., an anti-VEGF antibody (e.g., bevacizumab)) and/or the anti-TIGIT antagonist antibody (e.g., an anti-TIGIT antagonist antibody as disclosed herein, e.g., tiragolumab). In some instances, for example, following administration of the PD-1 axis binding antagonist (e.g., anti-PD-L1 antagonist antibody (e.g., atezolizumab)) and before administration of the VEGF antagonist (e.g., an anti-VEGF antibody (e.g., bevacizumab)) or the anti-TIGIT antagonist antibody (e.g., an anti-TIGIT antagonist antibody as disclosed herein, e.g., tiragolumab), the method includes an intervening first observation period. In some instances, for example, following administration of the PD-1 axis binding antagonist (e.g., anti-PD-L1 antagonist antibody (e.g., atezolizumab)), the VEGF antagonist (e.g., an anti-VEGF antibody (e.g., bevacizumab)) is administered to the subject or population of subjects before the anti-TIGIT antagonist antibody (e.g., an anti-TIGIT antagonist antibody as disclosed herein, e.g., tiragolumab). In other instances, following administration of the PD-1 axis binding antagonist (e.g., anti-PD-L1 antagonist antibody (e.g., atezolizumab)), the anti-TIGIT antagonist antibody (e.g., an anti-TIGIT antagonist antibody as disclosed herein, e.g., tiragolumab) is administered to the subject or population of subjects before the VEGF antagonist (e.g., an anti-VEGF antibody (e.g., bevacizumab)). In some instances, the PD-1 axis binding antagonist (e.g., anti-PD-L1 antagonist antibody (e.g., atezolizumab)) is first administered to the subject or population of subjects, the VEGF antagonist (e.g., an anti-VEGF antibody (e.g., bevacizumab)) is administered to the subject or population of subjects following administration of the PD-1 axis binding antagonist (e.g., anti-PD-L1 antagonist antibody (e.g., atezolizumab)), and the anti-TIGIT antagonist antibody (e.g., an anti-TIGIT antagonist antibody as disclosed herein, e.g., tiragolumab) is administered to the subject or population of subjects following administration of the VEGF antagonist (e.g., an anti-VEGF antibody (e.g., bevacizumab)) antagonist. In some instances, the PD-1 axis binding antagonist (e.g., anti-PD-L1 antagonist antibody (e.g., atezolizumab)) is first administered to the subject or population of subjects, the anti-TIGIT antagonist antibody (e.g., an anti-TIGIT antagonist antibody as disclosed herein, e.g., tiragolumab) is administered to the subject or population of subjects following administration of the PD-1 axis binding antagonist (e.g., anti-PD-L1 antagonist antibody (e.g., atezolizumab)), and the VEGF antagonist (e.g., an anti-VEGF antibody (e.g., bevacizumab)) is administered to the subject or population of subjects following administration of the anti-TIGIT antagonist antibody (e.g., an anti-TIGIT antagonist antibody as disclosed herein, e.g., tiragolumab).

In some instances, the method further includes a second observation period following administration of the VEGF antagonist (e.g., an anti-VEGF antibody (e.g., bevacizumab)) or the anti-TIGIT antagonist antibody (e.g., an anti-TIGIT antagonist antibody as disclosed herein, e.g., tiragolumab). In some instances, the method further includes a third observation period following administration of the PD-1 axis binding antagonist (e.g., anti-PD-L1 antagonist antibody (e.g., atezolizumab)), the VEGF antagonist (e.g., an anti-VEGF antibody (e.g., bevacizumab)), and the anti-TIGIT antagonist antibody (e.g., an anti-TIGIT antagonist antibody as disclosed herein, e.g., tiragolumab).

In some instances, the method includes both a first observation period following administration of the PD-1 axis binding antagonist (e.g., anti-PD-L1 antagonist antibody (e.g., atezolizumab)) and second observation period following administration of the VEGF antagonist (e.g., an anti-VEGF antibody (e.g., bevacizumab)) or the anti-TIGIT antagonist antibody (e.g., an anti-TIGIT antagonist antibody as disclosed herein, e.g., tiragolumab). In some instances, the method includes a first observation period following administration of the PD-1 axis binding antagonist (e.g., anti-PD-L1 antagonist antibody (e.g., atezolizumab)), a second observation period following administration of the VEGF antagonist (e.g., an anti-VEGF antibody (e.g., bevacizumab)), or the anti-TIGIT antagonist antibody (e.g., an anti-TIGIT antagonist antibody as disclosed herein, e.g., tiragolumab), and a third observation period following administration of the TIGIT antagonist antibody, the PD-1 axis binding antagonist, and the VEGF antagonist. In some instances, the first, second, and/or third observation periods are each between about 30 minutes to about 120 minutes in length (e.g., between about 30 minutes and 60 minutes in length, between 60 and 90 minutes in length, and/or between 90 and 120 minutes in length). In instances in which the first, second, and third observation periods are each about 60 minutes in length, the method may include recording the subject's vital signs (e.g., pulse rate, respiratory rate, blood pressure, and temperature) at about 30±10 minutes after administration of the PD-1 axis binding antagonist (e.g., anti-PD-L1 antagonist antibody (e.g., atezolizumab)), the anti-TIGIT antagonist antibody (e.g., an anti-TIGIT antagonist antibody as disclosed herein, e.g., tiragolumab), or the VEGF antagonist (e.g., an anti-VEGF antibody (e.g., bevacizumab)) during the first, second, or third observation periods. In instances in which the first, second, and third observation periods are each about 30 minutes in length, the method may include recording the subject's vital signs (e.g., pulse rate, respiratory rate, blood pressure, and temperature) at about 15±10 minutes after administration of the PD-1 axis binding antagonist (e.g., anti-PD-L1 antagonist antibody (e.g., atezolizumab)), the anti-TIGIT antagonist antibody (e.g., an anti-TIGIT antagonist antibody as disclosed herein, e.g., tiragolumab), or the VEGF antagonist (e.g., an anti-VEGF antibody (e.g., bevacizumab)) during the first, second, or third observation periods.

In some instances, the anti-TIGIT antagonist antibody (e.g., an anti-TIGIT antagonist antibody as disclosed herein, e.g., tiragolumab) is administered to the subject or population of subjects before the PD-1 axis binding antagonist (e.g., anti-PD-L1 antagonist antibody (e.g., atezolizumab)) or the VEGF antagonist (e.g., an anti-VEGF antibody (e.g., bevacizumab)).

In some instances, for example, following administration of the anti-TIGIT antagonist antibody and before administration of the PD-1 axis binding antagonist (e.g., anti-PD-L1 antagonist antibody (e.g., atezolizumab)) or the VEGF antagonist (e.g., an anti-VEGF antibody (e.g., bevacizumab)), the method includes an intervening first observation period. In some instances, for example, following administration of the anti-TIGIT antagonist antibody, the PD-1 axis binding antagonist (e.g., anti-PD-L1 antagonist antibody (e.g., atezolizumab)) is administered to the subject or population of subjects before the VEGF antagonist (e.g., an anti-VEGF antibody (e.g., bevacizumab)). In other instances, for example, following administration of the anti-TIGIT antagonist antibody, the VEGF antagonist (e.g., an anti-VEGF antibody (e.g., bevacizumab)) is administered to the subject or population of subjects before the PD-1 axis binding antagonist (e.g., anti-PD-L1 antagonist antibody (e.g., atezolizumab)). In some instances, the anti-TIGIT antagonist antibody (e.g., an anti-TIGIT antagonist antibody as disclosed herein, e.g., tiragolumab) is first administered to the subject or population of subjects, the PD-1 axis binding antagonist (e.g., anti-PD-L1 antagonist antibody (e.g., atezolizumab)) is administered to the subject or population of subjects following administration of the anti-TIGIT antagonist antibody (e.g., an anti-TIGIT antagonist antibody as disclosed herein, e.g., tiragolumab), and the VEGF antagonist (e.g., an anti-VEGF antibody (e.g., bevacizumab)) is administered to the subject or population of subjects following administration of the PD-1 axis binding antagonist (e.g., anti-PD-L1 antagonist antibody (e.g., atezolizumab)). In some instances, the anti-TIGIT antagonist antibody (e.g., an anti-TIGIT antagonist antibody as disclosed herein, e.g., tiragolumab) is first administered to the subject or population of subjects, the VEGF antagonist (e.g., an anti-VEGF antibody (e.g., bevacizumab)) is administered to the subject or population of subjects following administration of the anti-TIGIT antagonist antibody (e.g., an anti-TIGIT antagonist antibody as disclosed herein, e.g., tiragolumab), and the PD-1 axis binding antagonist (e.g., anti-PD-L1 antagonist antibody (e.g., atezolizumab)) is administered to the subject or population of subjects following administration of the VEGF antagonist (e.g., an anti-VEGF antibody (e.g., bevacizumab)).

In some instances, the method further includes a second observation period following administration of the PD-1 axis binding antagonist (e.g., anti-PD-L1 antagonist antibody (e.g., atezolizumab)) or the VEGF antagonist (e.g., an anti-VEGF antibody (e.g., bevacizumab)). In some instances, the method further includes a third observation period following administration of the anti-TIGIT antagonist antibody (e.g., an anti-TIGIT antagonist antibody as disclosed herein, e.g., tiragolumab), the PD-1 axis binding antagonist (e.g., anti-PD-L1 antagonist antibody (e.g., atezolizumab)), and the VEGF antagonist (e.g., an anti-VEGF antibody (e.g., bevacizumab)).

In some instances, the method includes both a first observation period following administration of the anti-TIGIT antagonist antibody and second observation period following administration of the PD-1 axis binding antagonist (e.g., anti-PD-L1 antagonist antibody (e.g., atezolizumab)) or VEGF antagonist (e.g., an anti-VEGF antibody (e.g., bevacizumab)). In some instances, the method includes a first observation period following administration of the anti-TIGIT antagonist antibody, a second observation period following administration of the PD-1 axis binding antagonist (e.g., anti-PD-L1 antagonist antibody (e.g., atezolizumab)) or the VEGF antagonist (e.g., an anti-VEGF antibody (e.g., bevacizumab)), and a third observation period following administration of the TIGIT antagonist antibody, the PD-1 axis binding antagonist, and the VEGF antagonist. In some instances, the first, second, and third observation periods are each between about 30 minutes to about 60 minutes in length. In instances in which the first, second, and third observation periods are each about 60 minutes in length, the method may include recording the subject or population of subjects's vital signs (e.g., pulse rate, respiratory rate, blood pressure, and temperature) at about 30±10 minutes after administration of the anti-TIGIT antagonist antibody, the PD-1 axis binding antagonist (e.g., anti-PD-L1 antagonist antibody (e.g., atezolizumab)), or the VEGF antagonist (e.g., an anti-VEGF antibody (e.g., bevacizumab)) during the first, second, or third observation periods. In instances in which the first, second, and third observation periods are each about 30 minutes in length, the method may include recording the subject or population of subjects's vital signs (e.g., pulse rate, respiratory rate, blood pressure, and temperature) at about 15±10 minutes after administration of the anti-TIGIT antagonist antibody, the PD-1 axis binding antagonist (e.g., anti-PD-L1 antagonist antibody (e.g., atezolizumab)), or the VEGF antagonist (e.g., an anti-VEGF antibody (e.g., bevacizumab)) during the first, second, or third observation periods.

In some instances, the VEGF antagonist (e.g., an anti-VEGF antibody (e.g., bevacizumab)) is administered to the subject or population of subjects before the anti-TIGIT antagonist antibody (e.g., an anti-TIGIT antagonist antibody as disclosed herein, e.g., tiragolumab) or the PD-1 axis binding antagonist (e.g., anti-PD-L1 antagonist antibody (e.g., atezolizumab)). In some instances, for example, following administration of the VEGF antagonist (e.g., an anti-VEGF antibody (e.g., bevacizumab)) and before administration of the anti-TIGIT antagonist antibody (e.g., an anti-TIGIT antagonist antibody as disclosed herein, e.g., tiragolumab) or the PD-1 axis binding antagonist (e.g., anti-PD-L1 antagonist antibody (e.g., atezolizumab)), the method includes an intervening first observation period. In some instances, for example, following administration of the VEGF antagonist (e.g., an anti-VEGF antibody (e.g., bevacizumab)), the anti-TIGIT antagonist antibody (e.g., an anti-TIGIT antagonist antibody as disclosed herein, e.g., tiragolumab) is administered to the subject or population of subjects before the PD-1 axis binding antagonist (e.g., anti-PD-L1 antagonist antibody (e.g., atezolizumab)). In other instances, for example, following administration of the VEGF antagonist (e.g., an anti-VEGF antibody (e.g., bevacizumab)), the PD-1 axis binding antagonist (e.g., anti-PD-L1 antagonist antibody (e.g., atezolizumab)) is administered to the subject or population of subjects before the anti-TIGIT antagonist antibody (e.g., an anti-TIGIT antagonist antibody as disclosed herein, e.g., tiragolumab). In some instances, the VEGF antagonist (e.g., an anti-VEGF antibody (e.g., bevacizumab)) is first administered to the subject or population of subjects, the anti-TIGIT antagonist antibody (e.g., an anti-TIGIT antagonist antibody as disclosed herein, e.g., tiragolumab) is administered to the subject or population of subjects following administration of the VEGF antagonist (e.g., an anti-VEGF antibody (e.g., bevacizumab)), and the PD-1 axis binding antagonist (e.g., anti-PD-L1 antagonist antibody (e.g., atezolizumab)) is administered to the subject or population of subjects following administration of the anti-TIGIT antagonist antibody (e.g., an anti-TIGIT antagonist antibody as disclosed herein, e.g., tiragolumab). In some instances, the VEGF antagonist (e.g., an anti-VEGF antibody (e.g., bevacizumab)) is first administered to the subject or population of subjects, the PD-1 axis binding antagonist (e.g., anti-PD-L1 antagonist antibody (e.g., atezolizumab)) is administered to the subject or population of subjects following administration of the VEGF antagonist (e.g., an anti-VEGF antibody (e.g., bevacizumab)), and the anti-TIGIT antagonist antibody (e.g., an anti-TIGIT antagonist antibody as disclosed herein, e.g., tiragolumab) is administered to the subject or population of subjects following administration of the PD-1 axis binding antagonist (e.g., anti-PD-L1 antagonist antibody (e.g., atezolizumab)) antagonist.

In some instances, the method further includes a second observation period following administration of the anti-TIGIT antagonist antibody (e.g., an anti-TIGIT antagonist antibody as disclosed herein, e.g., tiragolumab) or the PD-1 axis binding antagonist (e.g., anti-PD-L1 antagonist antibody (e.g., atezolizumab)). In some instances, the method further includes a third observation period following administration of the VEGF antagonist (e.g., an anti-VEGF antibody (e.g., bevacizumab)), the anti-TIGIT antagonist antibody (e.g., an anti-TIGIT antagonist antibody as disclosed herein, e.g., tiragolumab), and the PD-1 axis binding antagonist (e.g., anti-PD-L1 antagonist antibody (e.g., atezolizumab)).

In some instances, the method includes both a first observation period following administration of the VEGF antagonist (e.g., an anti-VEGF antibody (e.g., bevacizumab)) and second observation period following administration of the anti-TIGIT antagonist antibody (e.g., an anti-TIGIT antagonist antibody as disclosed herein, e.g., tiragolumab) or the PD-1 axis binding antagonist (e.g., anti-PD-L1 antagonist antibody (e.g., atezolizumab)). In some instances, the method includes a first observation period following administration of the VEGF antagonist (e.g., an anti-VEGF antibody (e.g., bevacizumab)), a second observation period following administration of the anti-TIGIT antagonist antibody (e.g., an anti-TIGIT antagonist antibody as disclosed herein, e.g., tiragolumab) or the PD-1 axis binding antagonist (e.g., anti-PD-L1 antagonist antibody (e.g., atezolizumab)), and a third observation period following administration of the TIGIT antagonist antibody, the PD-1 axis binding antagonist, and the VEGF antagonist. In some instances, the first, second, and third observation periods are each between about 30 minutes to about 60 minutes in length. In instances in which the first, second, and third observation periods are each about 60 minutes in length, the method may include recording the subject's vital signs (e.g., pulse rate, respiratory rate, blood pressure, and temperature) at about 30±10 minutes after administration of the VEGF antagonist (e.g., an anti-VEGF antibody (e.g., bevacizumab)), the anti-TIGIT antagonist antibody (e.g., an anti-TIGIT antagonist antibody as disclosed herein, e.g., tiragolumab), or the PD-1 axis binding antagonist (e.g., anti-PD-L1 antagonist antibody (e.g., atezolizumab)) during the first, second, or third observation periods. In instances in which the first, second, and third observation periods are each about 30 minutes in length, the method may include recording the subject's vital signs (e.g., pulse rate, respiratory rate, blood pressure, and temperature) at about 15±10 minutes after administration of the VEGF antagonist (e.g., an anti-VEGF antibody (e.g., bevacizumab)), the anti-TIGIT antagonist antibody (e.g., an anti-TIGIT antagonist antibody as disclosed herein, e.g., tiragolumab), or the PD-1 axis binding antagonist (e.g., anti-PD-L1 antagonist antibody (e.g., atezolizumab)) during the first, second, or third observation periods.

In other instances, the anti-TIGIT antagonist antibody (e.g., an anti-TIGIT antagonist antibody as disclosed herein, e.g., tiragolumab) and the VEGF antagonist (e.g., an anti-VEGF antibody (e.g., bevacizumab)) are administered to the subject or population of subjects simultaneously. In some instances, for example, following administration of the anti-TIGIT antagonist antibody and the VEGF antagonist (e.g., an anti-VEGF antibody (e.g., bevacizumab)) the method includes an observation period.

In other instances, the anti-TIGIT antagonist antibody (e.g., an anti-TIGIT antagonist antibody as disclosed herein, e.g., tiragolumab) and the PD-1 axis binding antagonist (e.g., anti-PD-L1 antagonist antibody (e.g., atezolizumab)) are administered to the subject or population of subjects simultaneously. In some instances, for example, following administration of the anti-TIGIT antagonist antibody and the PD-1 axis binding antagonist (e.g., anti-PD-L1 antagonist antibody (e.g., atezolizumab)) the method includes an observation period.

In other instances, the VEGF antagonist (e.g., an anti-VEGF antibody (e.g., bevacizumab)) and the PD-1 axis binding antagonist (e.g., anti-PD-L1 antagonist antibody (e.g., atezolizumab)) are administered to the subject or population of subjects simultaneously. In some instances, for example, following administration of the VEGF antagonist (e.g., an anti-VEGF antibody (e.g., bevacizumab)) and the PD-1 axis binding antagonist (e.g., anti-PD-L1 antagonist antibody (e.g., atezolizumab)) the method includes an observation period.

In some instances, the observation period is between about 30 minutes to about 60 minutes in length. In instances in which the observation period is about 60 minutes in length, the method may include recording the subject or population of subjects's vital signs (e.g., pulse rate, respiratory rate, blood pressure, and temperature) at about 30±10 minutes after administration. In instances in which the observation period is about 30 minutes in length, the method may include recording the subject's vital signs (e.g., pulse rate, respiratory rate, blood pressure, and temperature) at about 15±10 minutes after administration.

In some instances, the PD-1 axis binding antagonist (e.g., anti-PD-L1 antagonist antibody (e.g., atezolizumab)) is administered after the simultaneous administration of the anti-TIGIT antagonist antibody (e.g., an anti-TIGIT antagonist antibody as disclosed herein, e.g., tiragolumab) and the VEGF antagonist (e.g., an anti-VEGF antibody (e.g., bevacizumab)).

In some instances, the VEGF antagonist (e.g., an anti-VEGF antibody (e.g., bevacizumab)) is administered after the simultaneous administration of the anti-TIGIT antagonist antibody (e.g., an anti-TIGIT antagonist antibody as disclosed herein, e.g., tiragolumab) and the PD-1 axis binding antagonist (e.g., anti-PD-L1 antagonist antibody (e.g., atezolizumab)).

In some instances, the anti-TIGIT antagonist antibody (e.g., an anti-TIGIT antagonist antibody as disclosed herein, e.g., tiragolumab) is administered after the simultaneous administration of the PD-1 axis binding antagonist (e.g., anti-PD-L1 antagonist antibody (e.g., atezolizumab)) and the VEGF antagonist (e.g., an anti-VEGF antibody (e.g., bevacizumab)).

In some instances, for example, following administration of the VEGF antagonist (e.g., an anti-VEGF antibody (e.g., bevacizumab)), the anti-TIGIT antagonist antibody (e.g., an anti-TIGIT antagonist antibody as disclosed herein, e.g., tiragolumab), and the PD-1 axis binding antagonist (e.g., anti-PD-L1 antagonist antibody (e.g., atezolizumab)) the method includes a second observation period. In some instances, for example, following administration of the VEGF antagonist (e.g., an anti-VEGF antibody (e.g., bevacizumab)) and the PD-1 axis binding antagonist (e.g., anti-PD-L1 antagonist antibody (e.g., atezolizumab)), the method includes a second observation period. In some instances, the second observation period is between about 30 minutes to about 60 minutes in length. In instances in which the second observation period is about 60 minutes in length, the method may include recording the subject's vital signs (e.g., pulse rate, respiratory rate, blood pressure, and temperature) at about 30±10 minutes after administration. In instances in which the second observation period is about 30 minutes in length, the method may include recording the subject's vital signs (e.g., pulse rate, respiratory rate, blood pressure, and temperature) at about 15±10 minutes after administration.

G. Therapeutic Methods and Uses Relating to Urothelial Cancer

Urothelial Carcinoma

Urothelial carcinoma (UC) is the most common cancer of the urinary system worldwide. The majority of cases originate in the bladder. UC can be diagnosed as non-muscle invasive, muscle-invasive, or metastatic disease, with 1 in 3 new cases diagnosed as muscle-invasive disease (cT2-T4a Nx M0 according to tumor, node, and metastasis (TNM) classification). Muscle-invasive UC (MIUC) collectively refers to muscle-invasive bladder cancer (MIBC) and muscle-invasive urinary tract urothelial cancer (UTUC). In 2018, there were an estimated 549,393 new cases of bladder cancer and 199,922 deaths worldwide. In Europe, it was estimated that there were 197,110 new cases of bladder cancer and 64,970 deaths, including 164,450 new cases and 52,930 deaths in the 28 member states of the European Union. In the United States, in 2020, it is estimated that there will be 81,400 new cases of bladder cancer and 17,980 deaths. Patients diagnosed with UC in the United States have a median age of 73, the highest age at diagnosis of all tumor types.

There is a particularly pressing need for therapeutic approaches for treatment of MIBC. MIBC represents approximately 30% of new urothelial cancer cases. MIBC has a 5-year survival rate of 25-50% (European Association of Urology EAU Guidelines 2013), with little improvement seen over the past 30 years because of a lack of effective new treatments in this disease area (Surveillance, Epidemiology, and End Results (SEER) Program. Cancer stat facts: bladder cancer, 2020). Therefore, there is a high unmet need for improved medical intervention.

Methods and Uses for Treating Urothelial Carcinoma

Provided herein are methods and uses for treating UC (e.g., bladder cancer (e.g., MIBC)) in a subject or population of subjects comprising administering to the subject or population of subjects one or more dosing cycles of an effective amount of an anti-TIGIT antagonist antibody (e.g., an anti-TIGIT antagonist antibody as disclosed herein, e.g., tiragolumab) and a PD-1 axis binding antagonist (e.g., an anti-PD-L1 antagonist antibody, such as atezolizumab, or an anti-PD-1 antagonist antibody such as pembrolizumab). The subject is preferably a human. In some embodiments, the subject or population of subjects has not been previously treated with cancer immunotherapy.

The present invention includes methods and uses involving administration of an effective amount of an anti-TIGIT antagonist antibody (e.g., an anti-TIGIT antagonist antibody as disclosed herein, e.g., tiragolumab) and a PD-1 axis binding antagonist (e.g., an anti-PD-L1 antagonist antibody, such as atezolizumab, or an anti-PD-1 antagonist antibody, such as e.g., pembrolizumab) to a subject or population of subjects in need thereof every three weeks (e.g., on Day 1 of each 21-day dosing cycle).

The present invention includes methods and uses for treating a subject or population of subjects having an MIBC, the method comprising administering to the subject or population of subjects one or more dosing cycles of an anti-TIGIT antagonist antibody at a dose (e.g., a fixed dose) of between about 30 mg to about 1200 mg every three weeks and a PD-1 axis binding antagonist at a dose (e.g., a fixed dose) of between about 80 mg to about 1600 mg every three weeks, wherein the subject or population of subjects is ineligible for treatment with a platinum-based chemotherapeutic agent (e.g., cisplatin). In some aspects, the method comprises administering to the subject or population of subjects one or more dosing cycles of an anti-TIGIT antagonist antibody at a dose (e.g., a fixed dose) of between 30 mg to 1200 mg every three weeks and a PD-1 axis binding antagonist at a dose (e.g., a fixed dose) of between 80 mg to 1600 mg every three weeks, wherein the subject or population of subjects is ineligible for treatment with a platinum-based chemotherapeutic agent (e.g., cisplatin).

The present invention includes methods and uses for treating a subject or population of subjects having an MIBC, the method comprising administering to the subject or population of subjects one or more dosing cycles of an anti-TIGIT antagonist antibody at a dose (e.g., a fixed dose) of between about 30 mg to about 1200 mg every three weeks and a PD-1 axis binding antagonist at a dose (e.g., a fixed dose) of between about 80 mg to about 1600 mg every three weeks, wherein the subject or population of subjects has a creatinine clearance <60 mL/min, a greater than or equal to grade 2 hearing loss, and/or a greater than or equal to grade 2 neuropathy. In some aspects, the method comprises administering to the subject or population of subjects one or more dosing cycles of an anti-TIGIT antagonist antibody at a dose (e.g., a fixed dose) of between 30 mg to 1200 mg every three weeks and a PD-1 axis binding antagonist at a dose (e.g., a fixed dose) of between 80 mg to 1600 mg every three weeks, wherein the subject or population of subjects has a creatinine clearance <60 mL/min, a greater than or equal to grade 2 hearing loss, and/or a greater than or equal to grade 2 neuropathy.

The present invention includes methods and uses for treating a subject or population of subjects having an MIBC, the method comprising administering to the subject or population of subjects one or more dosing cycles of an anti-TIGIT antagonist antibody at a dose (e.g., a fixed dose) of between about 30 mg to about 1200 mg every three weeks and a PD-1 axis binding antagonist at a dose (e.g., a fixed dose) of between about 80 mg to about 1600 mg every three weeks, wherein the treatment is a perioperative treatment. In some aspects, the method comprises administering to the subject or population of subjects one or more dosing cycles of an anti-TIGIT antagonist antibody at a dose (e.g., a fixed dose) of between 30 mg to 1200 mg every three weeks and a PD-1 axis binding antagonist at a dose (e.g., a fixed dose) of between 80 mg to 1600 mg every three weeks, wherein the treatment is a perioperative treatment.

The present invention includes methods and uses for treating a subject or population of subjects having an operable MIBC, the method comprising administering to the subject or population of subjects one or more dosing cycles of an anti-TIGIT antagonist antibody at a dose (e.g., a fixed dose) of between about 30 mg to about 1200 mg every three weeks and a PD-1 axis binding antagonist at a dose (e.g., a fixed dose) of between about 80 mg to about 1600 mg every three weeks. In some aspects, the method comprises administering to the subject or population of subjects one or more dosing cycles of an anti-TIGIT antagonist antibody at a dose (e.g., a fixed dose) of between 30 mg to 1200 mg every three weeks and a PD-1 axis binding antagonist at a dose (e.g., a fixed dose) of between 80 mg to 1600 mg every three weeks.

The PD-1 axis binding antagonist anti-TIGIT antagonist antibody may be administered in any suitable manner known in the art. For example, the PD-1 axis binding antagonist and anti-TIGIT antagonist antibody may be administered sequentially (on different days) or concurrently (on the same day or during the same treatment cycle). In some instances, the PD-1 axis binding antagonist and anti-TIGIT antagonist antibody may be administered on the same day. In some instances, the PD-1 axis binding antagonist is administered before the anti-TIGIT antagonist antibody. In some instances, the PD-1 axis binding antagonist is administered after the anti-TIGIT antagonist antibody. In some instances, the PD-1 axis binding antagonist is administered simultaneously with the anti-TIGIT antagonist antibody. In some instances, the PD-1 axis binding antagonist may be administered prior to an anti-TIGIT antagonist antibody that is administered on the same day. In some instances, the PD-1 axis binding antagonist may be administered after to an anti-TIGIT antagonist antibody that is administered on the same day. In yet other instances, the PD-1 axis binding antagonist is administered at the same time as the anti-TIGIT antagonist antibody. In some instances, the PD-1 axis binding antagonist is in a separate composition as the anti-TIGIT antagonist antibody. In some instances, the PD-1 axis binding antagonist is in the same composition as the anti-TIGIT antagonist antibody. In some instances, the PD-1 axis binding antagonist is administered through a separate intravenous line from any other therapeutic agent administered to the patient on the same day. The PD-1 axis binding antagonist and anti-TIGIT antagonist antibody may be administered by the same route of administration or by different routes of administration. In some instances, the PD-1 axis binding antagonist is administered intravenously, intramuscularly, subcutaneously, topically, orally, transdermally, intraperitoneally, intraorbitally, by implantation, by inhalation, intrathecally, intraventricularly, or intranasally. In some instances, the anti-TIGIT antagonist antibody is administered intravenously, intramuscularly, subcutaneously, topically, orally, transdermally, intraperitoneally, intraorbitally, by implantation, by inhalation, intrathecally, intraventricularly, or intranasally. In some instances, the anti-TIGIT antagonist antibody is administered intravenously, intramuscularly, subcutaneously, topically, orally, transdermally, intraperitoneally, intraorbitally, by implantation, by inhalation, intrathecally, intraventricularly, or intranasally. In some instances, the anti-TIGIT antagonist antibody is administered intravenously, intramuscularly, subcutaneously, topically, orally, transdermally, intraperitoneally, intraorbitally, by implantation, by inhalation, intrathecally, intraventricularly, or intranasally. In some instances, there is a first observation period following administration of the PD-1 axis binding antagonist. In some instances, there is a second observation period following administration of the PD-1 axis binding antagonist. In some instances, there is a first observation period following administration of the anti-TIGIT antagonist antibody. In some instances, there is a second observation period following administration of the anti-TIGIT antagonist antibody. In some instances, the observation period is between about 30 minutes to about 60 minutes in length. In some instances, the anti-TIGIT antagonist antibody and/or PD-1 axis binding antagonist are administered intravenously or subcutaneously. In some instances, the intravenous infusion is over 30±10 minutes and/or over 60±15 minutes. In one example, atezolizumab may be administered intravenously over 60 minutes; if the first infusion is tolerated, all subsequent infusions may be delivered over 30 minutes. In some examples, the PD-1 axis binding antagonist is not administered as an intravenous push or bolus. In one example, tiragolumab may be administered intravenously over 60 minutes; if the first infusion is tolerated, all subsequent infusions may be delivered over 30 minutes. In some examples, the anti-TIGIT antagonist antibody is not administered as an intravenous push or bolus.

In some instances, the first dosing cycle is initiated prior to a surgery. In some instances, one or more dosing cycles are completed prior to a surgery. In some instances, at least 1, 2, or 3 dosing cycles (e.g., 1, 2, 3, 4, 5, 6, 7, 8, or more dosing cycles) are completed prior to a surgery. In some instances, one or more dosing cycles (e.g., 1, 2, 3, 4, 5, 6, 7, 8, 9, 10, 11, 12, 13, 14, 15, 16, 17, 18, 19, 20, or more dosing cycles) are initiated after a surgery. In some instances, 1-17 dosing cycles (e.g., 1, 2, 3, 4, 5, 6, 7, 8, 9, 10, 11, 12, 13, 14, 15, 16, or 17 dosing cycles) are completed after the surgery. In some instances, at least one dosing cycle is initiated between about 4-6 weeks (e.g., about 4 weeks, about 5 weeks, or about 6 weeks) after the surgery. In some instances, the treatment includes a surgery. In some instances, the surgery is a cystectomy and/or lymph node dissection.

In some instances, administration of the effective amount of the anti-TIGIT antagonist antibody (e.g., an anti-TIGIT antagonist antibody as disclosed herein, e.g., tiragolumab) and the PD-1 axis binding antagonist (e.g., an anti-PD-L1 antagonist antibody, such as atezolizumab) results in a pCR. In some instances, administration of the effective amount of the anti-TIGIT antagonist antibody (e.g., an anti-TIGIT antagonist antibody as disclosed herein, e.g., tiragolumab) and the PD-1 axis binding antagonist (e.g., an anti-PD-L1 antagonist antibody, such as atezolizumab) results in an increase in recurrence-free survival (RFS), e.g., landmark RFS (e.g., landmark RFS at 12, 18, or 24 months). In some instances, administration of the effective amount of the anti-TIGIT antagonist antibody (e.g., an anti-TIGIT antagonist antibody as disclosed herein, e.g., tiragolumab) and the PD-1 axis binding antagonist (e.g., an anti-PD-L1 antagonist antibody, such as atezolizumab) results in an increase in event-free survival (EFS), e.g., landmark EFS (e.g., landmark EFS at 12, 18, or 24 months). In some instances, administration of the effective amount of the anti-TIGIT antagonist antibody (e.g., an anti-TIGIT antagonist antibody as disclosed herein, e.g., tiragolumab) and the PD-1 axis binding antagonist (e.g., an anti-PD-L1 antagonist antibody, such as atezolizumab) results in an increase in OS, e.g., landmark OS (e.g., landmark OS at 12, 18, or 24 months). In some instances, administration of the effective amount of the anti-TIGIT antagonist antibody (e.g., an anti-TIGIT antagonist antibody as disclosed herein, e.g., tiragolumab) and the PD-1 axis binding antagonist (e.g., an anti-PD-L1 antagonist antibody, such as atezolizumab) results in an increase in pathological downstaging rate. In some instances, administration of the effective amount of the anti-TIGIT antagonist antibody (e.g., an anti-TIGIT antagonist antibody as disclosed herein, e.g., tiragolumab) and the PD-1 axis binding antagonist (e.g., an anti-PD-L1 antagonist antibody, such as atezolizumab) results in an increase in pCR of the subject or population of subjects, e.g., as compared to treatment with the PD-1 axis binding antagonist without the anti-TIGIT antagonist antibody or as compared to treatment with the anti-TIGIT antagonist antibody without the PD-1 axis binding antagonist. In some instances, the effective amount of the anti-TIGIT antagonist antibody (e.g., an anti-TIGIT antagonist antibody as disclosed herein, e.g., tiragolumab) and the PD-1 axis binding antagonist (e.g., an anti-PD-L1 antagonist antibody, such as atezolizumab) extends EFS (e.g., landmark EFS) of the subject or population of subjects, e.g., as compared to treatment with the PD-1 axis binding antagonist without the anti-TIGIT antagonist antibody or as compared to treatment with the anti-TIGIT antagonist antibody without the PD-1 axis binding antagonist. In some instances, the effective amount of the anti-TIGIT antagonist antibody (e.g., an anti-TIGIT antagonist antibody as disclosed herein, e.g., tiragolumab) and the PD-1 axis binding antagonist (e.g., an anti-PD-L1 antagonist antibody, such as atezolizumab) extends RFS (e.g., landmark RFS) of the subject or population of subjects, e.g., as compared to treatment with the PD-1 axis binding antagonist without the anti-TIGIT antagonist antibody or as compared to treatment with the anti-TIGIT antagonist antibody without the PD-1 axis binding antagonist. In some instances, the effective amount of the anti-TIGIT antagonist antibody (e.g., an anti-TIGIT antagonist antibody as disclosed herein, e.g., tiragolumab) and the PD-1 axis binding antagonist (e.g., an anti-PD-L1 antagonist antibody, such as atezolizumab) extends OS (e.g., landmark OS) of the subject or population of subjects, e.g., as compared to treatment with the PD-1 axis binding antagonist without the anti-TIGIT antagonist antibody or as compared to treatment with the anti-TIGIT antagonist antibody without the PD-1 axis binding antagonist.

The present invention includes methods and uses involving administration of an effective amount of an anti-TIGIT antagonist antibody (e.g., an anti-TIGIT antagonist antibody as disclosed herein, e.g., tiragolumab) to a subject or population of subjects in need thereof every four weeks (e.g., on Day 1 of each 28-day dosing cycle). In some instances, the anti-TIGIT antagonist antibody (e.g., an anti-TIGIT antagonist antibody as disclosed herein, e.g., tiragolumab) is administered every four weeks (e.g., on Day 1 of each 28-day dosing cycle) and the PD-1 axis binding antagonist (e.g., an anti-PD-L1 antagonist antibody, e.g., atezolizumab, or an anti-PD-1 antagonist antibody, such as e.g., pembrolizumab) is administered every two weeks (e.g., on Days 1 and 15 of each 28-day dosing cycle), every three weeks (e.g., on Day 1 of each 21-day dosing cycle), or every four weeks (e.g., on Day 1 of each 28-day dosing cycle). In some instances, administration of the effective amount of the anti-TIGIT antagonist antibody (e.g., an anti-TIGIT antagonist antibody as disclosed herein, e.g., tiragolumab) results in a CR or a PR. In some instances, administration of the effective amount of the anti-TIGIT antagonist antibody (e.g., an anti-TIGIT antagonist antibody as disclosed herein, e.g., tiragolumab) results in an increase in pCR of the subject or population of subjects compared to a reference. In some instances, administration of the effective amount of the anti-TIGIT antagonist antibody (e.g., an anti-TIGIT antagonist antibody as disclosed herein, e.g., tiragolumab) results in an increase in EFS and/or RFS. In some instances, administration of the effective amount of the anti-TIGIT antagonist antibody (e.g., an anti-TIGIT antagonist antibody as disclosed herein, e.g., tiragolumab) extends OS of the subject or population of subjects.

The present invention includes methods and uses involving administration of an effective amount of an anti-TIGIT antagonist antibody (e.g., an anti-TIGIT antagonist antibody as disclosed herein, e.g., tiragolumab) and a PD-1 axis binding antagonist (e.g., an anti-PD-L1 antagonist antibody, such as atezolizumab, or an anti-PD-1 antagonist antibody, such as e.g., pembrolizumab) to a subject or population of subjects in need thereof every two weeks (e.g., on Days 1 and 15 of each 28-day dosing cycle). In some instances, administration of the effective amount of the anti-TIGIT antagonist antibody (e.g., an anti-TIGIT antagonist antibody as disclosed herein, e.g., tiragolumab) and the PD-1 axis binding antagonist (e.g., an anti-PD-L1 antagonist antibody, such as atezolizumab) results in a pCR. In some instances, administration of the effective amount of the anti-TIGIT antagonist antibody (e.g., an anti-TIGIT antagonist antibody as disclosed herein, e.g., tiragolumab) and the PD-1 axis binding antagonist (e.g., an anti-PD-L1 antagonist antibody, such as atezolizumab) results in an increase in EFS and/or RFS of the subject or population of subjects, e.g., as compared to treatment with the PD-1 axis binding antagonist without the anti-TIGIT antagonist antibody or as compared to treatment with the anti-TIGIT antagonist antibody without the PD-1 axis binding antagonist. In some instances, administration of the effective amount of the anti-TIGIT antagonist antibody (e.g., an anti-TIGIT antagonist antibody as disclosed herein, e.g., tiragolumab) and the PD-1 axis binding antagonist (e.g., an anti-PD-L1 antagonist antibody, such as atezolizumab) extends OS of the subject or population of subjects, e.g., as compared to treatment with the PD-1 axis binding antagonist without the anti-TIGIT antagonist antibody or as compared to treatment with the anti-TIGIT antagonist antibody without the PD-1 axis binding antagonist.

In certain instances, the present invention includes methods and uses involving administration of an effective amount of an anti-TIGIT antagonist antibody (e.g., an anti-TIGIT antagonist antibody as disclosed herein, e.g., tiragolumab) to a subject or population of subjects in need thereof every two weeks (e.g., on Days 1 and 15 of each 28-day dosing cycle). In some instances, administration of the effective amount of the anti-TIGIT antagonist antibody (e.g., an anti-TIGIT antagonist antibody as disclosed herein, e.g., tiragolumab) results in a pCR. In some instances, administration of the effective amount of the anti-TIGIT antagonist antibody (e.g., an anti-TIGIT antagonist antibody as disclosed herein, e.g., tiragolumab) results in an increase in EFS and/or RFS of the subject or population of subjects compared to a reference. In some instances, administration of the effective amount of the anti-TIGIT antagonist antibody (e.g., an anti-TIGIT antagonist antibody as disclosed herein, e.g., tiragolumab) extends OS of the subject or population of subjects.

In some instances, the subject or population of subjects receiving the anti-TIGIT antagonist antibody (e.g., an anti-TIGIT antagonist antibody as disclosed herein, e.g., tiragolumab) and the PD-1 axis binding antagonist (e.g., anti-PD-L1 antagonist antibody, such as atezolizumab) is being treated for an MIBC.

In some instances, the treatment may further comprise an additional therapy. Any suitable additional therapy known in the art or described herein may be used. The additional therapy may be radiation therapy, surgery (e.g., cystectomy), gene therapy, DNA therapy, viral therapy, RNA therapy, immunotherapy, bone marrow transplantation, nanotherapy, monoclonal antibody therapy, gamma irradiation, or a combination of the foregoing.

In some instances, the additional therapy is the administration of side-effect limiting agents (e.g., agents intended to lessen the occurrence and/or severity of side effects of treatment, such as anti-nausea agents, a corticosteroid (e.g., prednisone or an equivalent, e.g., at a dose of 1-2 mg/kg/day), hormone replacement medicine(s), and the like).

In any of the preceding examples, each dosing cycle may have any suitable length, e.g., about 7 days, about 14 days, about 21 days, about 28 days, or longer. In some instances, each dosing cycle is about 21 days.

Also provided herein are methods for treating MIBC in a subject or population of subjects comprising administering to the subject or population of subjects a treatment regimen comprising an effective amount of a PD-1 axis binding antagonist (e.g., atezolizumab) and/or anti-TIGIT antagonist antibody (e.g., an anti-TIGIT antagonist antibody as disclosed herein, e.g., tiragolumab) in combination with another anti-cancer agent or cancer therapy. For example, a PD-1 axis binding antagonist may be administered in combination with an additional chemotherapy or chemotherapeutic agent (see definition above); a targeted therapy or targeted therapeutic agent; an immunotherapy or immunotherapeutic agent, for example, a monoclonal antibody; one or more cytotoxic agents (see definition above); or combinations thereof.

In some instances in which the patient has a metastatic urothelial carcinoma (mUC) and the mUC has progressed during or following a platinum-containing therapy, the methods provided herein further comprise administering to the subject or population of subjects a second dosing regimen after the subject or population of subjects has experienced disease progression or unacceptable toxicity. In some instances, the second dosing regimen comprises one or more dosing cycles of a PD-1 axis binding antagonist and an antibody-drug conjugate (ADC). In some instances, the ADC is (a) enfortumab vedotin or (b) sacituzumab govitecan.

Also provided herein are methods for treating a subject or population of subjects having a mUC, the method comprising administering to the subject or population of subjects a first dosing regimen followed by a second dosing regimen, wherein (a) the first dosing regimen comprises one or more dosing cycles of tiragolumab at a dose (e.g., a fixed dose) of about 600 mg every three weeks and atezolizumab at a dose (e.g., a fixed dose) of about 1200 mg every three weeks; and (b) the second dosing regimen comprises one or more dosing cycles of atezolizumab at a dose (e.g., a fixed dose) of about 1200 mg every three weeks and (i) enfortumab vedotin is administered at a dose of 1.25 mg/kg every week for 2-weeks on/1 week off or (ii) sacituzumab govitecan is administered at a dose of 10 mg/kg every week for 2-weeks on/1 week off, wherein the second dosing regimen is administered to the subject or population of subjects after the subject or population of subjects has experienced disease progression or unacceptable toxicity during the first dosing regimen. In some aspects, provided herein are methods for treating a subject or population of subjects having a mUC, the method comprising administering to the subject or population of subjects a first dosing regimen followed by a second dosing regimen, wherein (a) the first dosing regimen comprises one or more dosing cycles of tiragolumab at a dose (e.g., a fixed dose) of 600 mg every three weeks and atezolizumab at a dose (e.g., a fixed dose) of 1200 mg every three weeks; and (b) the second dosing regimen comprises one or more dosing cycles of atezolizumab at a dose (e.g., a fixed dose) of 1200 mg every three weeks and (i) enfortumab vedotin is administered at a dose of 1.25 mg/kg every week for 2-weeks on/1 week off or (ii) sacituzumab govitecan is administered at a dose of 10 mg/kg every week for 2-weeks on/1 week off, wherein the second dosing regimen is administered to the subject or population of subjects after the subject or population of subjects has experienced disease progression or unacceptable toxicity during the first dosing regimen.

In some instances, the treatment results in an ORR of the population of subjects of at least about 13.4% to at least about 15% (e.g., at least about 13.5%, 14%, 14.5%, or 15%).

In some instances, the treatment results in a median OS of the population of subjects of at least about 7.9 months (e.g., 8.0 months, 8.1 months, 8.2 months, 8.3 months, 8.4 months, 8.5 months, 8.6 months, 8.7 months, 8.8 months, 8.9 months, 9 months, 9.5 months, 10 months, 11 months, 12 months, 13 months, or 14 months). In some instances, the treatment results in a median OS of the population of subjects of at least about 8.6 months (e.g., 8.6 months, 8.7 months, 8.8 months, 8.9 months, 9 months, 9.5 months, 10 months, 11 months, 12 months, 13 months, 14 months, 15 months, or 16 months). In some instances, the treatment results in a median OS of the population of subjects of about 7.9 months to about 8.6 months (e.g., about 8.0 months, 8.1 months, 8.2 months, 8.3 months, 8.4 months, 8.5 months, or 8.6 months).

In some instances, the treatment results in an ORR of the population of subjects of at least about 13.4% to at least about 31% (e.g., at least about 13.5%, 14%, 15%, 18%, 17%, 18%, 19%, 20%, 21%, 22%, 23%, 24%, 25%, 26%, 27%, 28%, 29%, 30%, or 31%). In some instances, the treatment results in an ORR of the population of subjects of at least about 31% (e.g., between about 31% and about 100%, e.g., between about 31% and about 60% (e.g., 35%, 40%, 45%, 50%, 55%, or 60%).

In some instances, the treatment results in a median OS of the population of subjects of at least about 7.9 (e.g., between about 7.9 and about 36 months (e.g., between about 7.9 and about 24 months (e.g., 8 months, 10 months, 12 months, 14 months, 16 months, 18 months, 20 months, 22 months, or 24 months))). In some instances, the treatment results in a median OS of the population of subjects of at least about 16.3 months (e.g., between about 16.3 months and 36 months (e.g., between about 16.3 and about 24 months (e.g., 16 months, 17 months, 18 months, 19 months, 20 months, 21 months, 22 months, 23 months, or 24 months)). In some instances, the treatment results in a median OS of the population of subjects of about 7.9 months to about 16.3 months (e.g., 8 months, 8.5 months, 9 months, 9.5 months, 10 months, 10.5 months, 11 months, 11.5 months, 12 months, 12.5 months, 13 months, 13.5 months, 14 months, 14.5 months, 15 months, 15.5 months, 16 months, or 16.3 months, e.g., 7.9-9 months, 9-10 months, 10-11 months, 11-12 months, 12-13 months, 13-14 months, 14-15 months, 15-16 months, or more than 16 months).

Dosing of Anti-TIGIT Antagonist Antibodies

Dosing of anti-TIGIT antagonist antibodies is described in Section III(K).

Dosing of PD-1 Axis Binding Antagonists

Dosing of PD-1 axis binding antagonists is described in Section III(K).

Cancer Characterization and Selection

In some instances, in any of the methods, uses, or compositions for use described herein, the UC (e.g., bladder cancer (e.g., MIBC)) is surgically operable (e.g., an UC (e.g., bladder cancer (e.g., MIBC)) fit for cystectomy). In some instances, in any of the methods, uses, or compositions for use described herein, the MIBC is surgically operable (e.g., an MIBC fit for cystectomy).

In some instances, in any of the methods, uses, or compositions for use described herein, the subject is ineligible for platinum-based chemotherapy (e.g., cisplatin-ineligible). In some instances, the subject is cisplatin-ineligible. In some instances, the subject has a creatinine clearance <60 mL/min. In some instances, the subject has a creatinine clearance of 30 mL/min. In some instances, the subject has a greater than or equal to grade 2 hearing loss. In some instances, the subject has a greater than or equal to grade 2 neuropathy. In some instances, a subject that has a creatinine clearance <60 mL/min, a greater than or equal to grade 2 hearing loss, and/or a greater than or equal to grade 2 neuropathy is cisplatin-ineligible. In some instances, a subject that has a creatinine clearance <60 mL/min is cisplatin-ineligible. In some instances, a subject that has a greater than or equal to grade 2 hearing loss is cisplatin-ineligible. In some instances, a subject that has a greater than or equal to grade 2 neuropathy is cisplatin-ineligible. In some instances, a subject that refuses cisplatin-based chemotherapy is cisplatin-ineligible. In some instances, the subject has an Eastern Cooperative Oncology Group (ECOG) Performance Status (PS) of 0 or 1.

In some instances, in any of the methods, uses, or compositions for use described herein, the presence or level of circulating tumor DNA (ctDNA) may be assessed. In some instances, ctDNA is assessed in a sample (e.g., a blood sample) from the subject. In some instances, ctDNA is assessed in a sample from the subject prior to day 1 of the first dosing cycle (e.g., the first dosing cycle of an anti-TIGIT antagonist antibody and a PD-1 axis binding antagonist). In some instances, ctDNA is assessed in a sample from the subject prior to surgery (e.g., a cystectomy). In some instances, ctDNA is assessed in a sample from the subject after surgery (e.g., a cystectomy). In some instances, ctDNA is assessed in a sample from the subject 4-6 weeks (e.g., 4 weeks, 5 weeks, or 6 weeks) after surgery (e.g., a cystectomy). In some instances, ctDNA is assessed in a sample from the subject 6 months after surgery (e.g., a cystectomy).

Assessment of PD-L1 Expression

The expression of PD-L1 may be assessed as described in Section III(L).

Responses to Treatment

In some embodiments of any of the methods described herein, a subject's response to the therapy can be characterized by one or more measures. In some embodiments, the treatment results in a pCR. In some embodiments, the treatment results in an increase in recurrence-free survival (RFS), event-free survival (EFS), or OS. In some embodiments, the treatment results in an increase in landmark RFS, landmark EFS, or landmark OS. In some embodiments, the treatment results in an increase in pathological downstaging rate.

In some instances, the treatment results in an increase in pCR of the subject, e.g., as compared to treatment with the PD-1 axis binding antagonist without the anti-TIGIT antagonist antibody or as compared to treatment with the anti-TIGIT antagonist antibody without the PD-1 axis binding antagonist.

In some instances, the treatment extends OS of the subject, e.g., as compared to treatment with the PD-1 axis binding antagonist without the anti-TIGIT antagonist antibody or as compared to treatment with the anti-TIGIT antagonist antibody without the PD-1 axis binding antagonist. In some instances, the treatment extends EFS of the subject, e.g., as compared to treatment with the PD-1 axis binding antagonist without the anti-TIGIT antagonist antibody or as compared to treatment with the anti-TIGIT antagonist antibody without the PD-1 axis binding antagonist. In some instances, the treatment extends RFS of the subject, e.g., as compared to treatment with the PD-1 axis binding antagonist without the anti-TIGIT antagonist antibody or as compared to treatment with the anti-TIGIT antagonist antibody without the PD-1 axis binding antagonist. In some instances, the treatment increases pathological downstaging rate of the subject, e.g., as compared to treatment with the PD-1 axis binding antagonist without the anti-TIGIT antagonist antibody or as compared to treatment with the anti-TIGIT antagonist antibody without the PD-1 axis binding antagonist.

In some embodiments, a treatment described herein extends the pCR of the subject by at least about 2 months (e.g., by 2-120 months, by 2.5-100 months, by 3.0-80 months, by 4.0-60 months, by 5.0-48 months, by 6.0-36 months, by 8.0-24 months, or by 10-12 months, e.g., by at least about 2.4 months, 2.5 months, 2.6 months, 2.7 months, 2.8 months, 2.9 months, 3.0 months, 3.1 months, 3.2 months, 3.3 months, 3.4 months, 3.5 months, 3.6 months, 3.7 months, 3.8 months, 3.9 months, 4.0 months, 4.1 months, 4.2 months, 4.3 months, 4.4 months, 4.5 months, 4.6 months, 4.7 months, 4.8 months, 4.9 months, 5.0 months, 5.1 months, 5.2 months, 5.3 months, 5.4 months, 5.5 months, 5.6 months, 5.7 months, 5.8 months, 5.9 months, 6.0 months, 6.5 months, 7.0 months, 7.5 months, 8.0 months, 8.5 months, 9.0 months, 9.5 months, 10 months, 10.5 months, 11 months, 11.5 months, 12 months, 13 months, 14 months, 15 months, 16 months, 17 months, 18 months, 19 months, 20 months, 21 months, 22 months, 23 months, 24 months, 25 months, 26 months, 27 months, 28 months, 29 months, 30 months, 31 months, 32 months, 33 months, 34 months, 35 months, or 36 months). In some embodiments, the treatment extends the pCR of the subject by at least about 4 months (e.g., by 4-120 months, by 5-100 months, by 6-80 months, by 7-60 months, by 8-48 months, by 9-36 months, or by 10-24 months, e.g., by at least about 4.0 months, 4.1 months, 4.2 months, 4.3 months, 4.4 months, 4.5 months, 4.6 months, 4.7 months, 4.8 months, 4.9 months, 5.0 months, 5.1 months, 5.2 months, 5.3 months, 5.4 months, 5.5 months, 5.6 months, 5.7 months, 5.8 months, 5.9 months, 6.0 months, 6.5 months, 7.0 months, 7.5 months, 8.0 months, 8.5 months, 9.0 months, 9.5 months, 10 months, 10.5 months, 11 months, 11.5 months, 12 months, 13 months, 14 months, 15 months, 16 months, 17 months, 18 months, 19 months, 20 months, 21 months, 22 months, 23 months, 24 months, 25 months, 26 months, 27 months, 28 months, 29 months, 30 months, 31 months, 32 months, 33 months, 34 months, 35 months, or 36 months).

In some embodiments, a treatment described herein extends the EFS of the subject by at least about 2 months (e.g., by 2-120 months, by 2.5-100 months, by 3.0-80 months, by 4.0-60 months, by 5.0-48 months, by 6.0-36 months, by 8.0-24 months, or by 10-12 months, e.g., by at least about 2.4 months, 2.5 months, 2.6 months, 2.7 months, 2.8 months, 2.9 months, 3.0 months, 3.1 months, 3.2 months, 3.3 months, 3.4 months, 3.5 months, 3.6 months, 3.7 months, 3.8 months, 3.9 months, 4.0 months, 4.1 months, 4.2 months, 4.3 months, 4.4 months, 4.5 months, 4.6 months, 4.7 months, 4.8 months, 4.9 months, 5.0 months, 5.1 months, 5.2 months, 5.3 months, 5.4 months, 5.5 months, 5.6 months, 5.7 months, 5.8 months, 5.9 months, 6.0 months, 6.5 months, 7.0 months, 7.5 months, 8.0 months, 8.5 months, 9.0 months, 9.5 months, 10 months, 10.5 months, 11 months, 11.5 months, 12 months, 13 months, 14 months, 15 months, 16 months, 17 months, 18 months, 19 months, 20 months, 21 months, 22 months, 23 months, 24 months, 25 months, 26 months, 27 months, 28 months, 29 months, 30 months, 31 months, 32 months, 33 months, 34 months, 35 months, or 36 months). In some embodiments, the treatment extends the EFS of the subject by at least about 4 months (e.g., by 4-120 months, by 5-100 months, by 6-80 months, by 7-60 months, by 8-48 months, by 9-36 months, or by 10-24 months, e.g., by at least about 4.0 months, 4.1 months, 4.2 months, 4.3 months, 4.4 months, 4.5 months, 4.6 months, 4.7 months, 4.8 months, 4.9 months, 5.0 months, 5.1 months, 5.2 months, 5.3 months, 5.4 months, 5.5 months, 5.6 months, 5.7 months, 5.8 months, 5.9 months, 6.0 months, 6.5 months, 7.0 months, 7.5 months, 8.0 months, 8.5 months, 9.0 months, 9.5 months, 10 months, 10.5 months, 11 months, 11.5 months, 12 months, 13 months, 14 months, 15 months, 16 months, 17 months, 18 months, 19 months, 20 months, 21 months, 22 months, 23 months, 24 months, 25 months, 26 months, 27 months, 28 months, 29 months, 30 months, 31 months, 32 months, 33 months, 34 months, 35 months, or 36 months).

In some embodiments, a treatment described herein extends the RFS of the subject by at least about 2 months (e.g., by 2-120 months, by 2.5-100 months, by 3.0-80 months, by 4.0-60 months, by 5.0-48 months, by 6.0-36 months, by 8.0-24 months, or by 10-12 months, e.g., by at least about 2.4 months, 2.5 months, 2.6 months, 2.7 months, 2.8 months, 2.9 months, 3.0 months, 3.1 months, 3.2 months, 3.3 months, 3.4 months, 3.5 months, 3.6 months, 3.7 months, 3.8 months, 3.9 months, 4.0 months, 4.1 months, 4.2 months, 4.3 months, 4.4 months, 4.5 months, 4.6 months, 4.7 months, 4.8 months, 4.9 months, 5.0 months, 5.1 months, 5.2 months, 5.3 months, 5.4 months, 5.5 months, 5.6 months, 5.7 months, 5.8 months, 5.9 months, 6.0 months, 6.5 months, 7.0 months, 7.5 months, 8.0 months, 8.5 months, 9.0 months, 9.5 months, 10 months, 10.5 months, 11 months, 11.5 months, 12 months, 13 months, 14 months, 15 months, 16 months, 17 months, 18 months, 19 months, 20 months, 21 months, 22 months, 23 months, 24 months, 25 months, 26 months, 27 months, 28 months, 29 months, 30 months, 31 months, 32 months, 33 months, 34 months, 35 months, or 36 months). In some embodiments, the treatment extends the RFS of the subject by at least about 4 months (e.g., by 4-120 months, by 5-100 months, by 6-80 months, by 7-60 months, by 8-48 months, by 9-36 months, or by 10-24 months, e.g., by at least about 4.0 months, 4.1 months, 4.2 months, 4.3 months, 4.4 months, 4.5 months, 4.6 months, 4.7 months, 4.8 months, 4.9 months, 5.0 months, 5.1 months, 5.2 months, 5.3 months, 5.4 months, 5.5 months, 5.6 months, 5.7 months, 5.8 months, 5.9 months, 6.0 months, 6.5 months, 7.0 months, 7.5 months, 8.0 months, 8.5 months, 9.0 months, 9.5 months, 10 months, 10.5 months, 11 months, 11.5 months, 12 months, 13 months, 14 months, 15 months, 16 months, 17 months, 18 months, 19 months, 20 months, 21 months, 22 months, 23 months, 24 months, 25 months, 26 months, 27 months, 28 months, 29 months, 30 months, 31 months, 32 months, 33 months, 34 months, 35 months, or 36 months).

In some embodiments, OS is measured as the period of time from the start of treatment to death. In some instances, the treatment extends the OS of the subject by at least about 2 months (e.g., by 2-120 months, by 3-110 months, by 4-100 months, by 5-80 months, by 6-60 months, by 7-48 months, by 8-36 months, or by 10-24 months, e.g., by at least about 2 months, 2.1 months, 2.2 months, 2.3 months, 2.4 months, 2.5 months, 2.6 months, 2.7 months, 2.8 months, 2.9 months, 3.0 months, 3.1 months, 3.2 months, 3.3 months, 3.4 months, 3.5 months, 3.6 months, 3.7 months, 3.8 months, 3.9 months, 4.0 months, 4.1 months, 4.2 months, 4.3 months, 4.4 months, 4.5 months, 4.6 months, 4.7 months, 4.8 months, 4.9 months, 5.0 months, 5.1 months, 5.2 months, 5.3 months, 5.4 months, 5.5 months, 5.6 months, 5.7 months, 5.8 months, 5.9 months, 6.0 months, 6.5 months, 7.0 months, 7.5 months, 8.0 months, 8.5 months, 9.0 months, 9.5 months, 10 months, 10.5 months, 11 months, 11.5 months, 12 months, 13 months, 14 months, 15 months, 16 months, 17 months, 18 months, 19 months, 20 months, 21 months, 22 months, 23 months, 24 months, 25 months, 26 months, 27 months, 28 months, 29 months, 30 months, 31 months, 32 months, 33 months, 34 months, 35 months, or 36 months). In some instances, the treatment extends the OS of the subject by at least about 3.3 months (e.g., by 3.3-120 months, by 4-100 months, by 5-80 months, by 6-60 months, by 7-48 months, by 8-36 months, or by 10-24 months, e.g., by at least about 3.3 months, 3.4 months, 3.5 months, 3.6 months, 3.7 months, 3.8 months, 3.9 months, 4.0 months, 4.1 months, 4.2 months, 4.3 months, 4.4 months, 4.5 months, 4.6 months, 4.7 months, 4.8 months, 4.9 months, 5.0 months, 5.1 months, 5.2 months, 5.3 months, 5.4 months, 5.5 months, 5.6 months, 5.7 months, 5.8 months, 5.9 months, 6.0 months, 6.5 months, 7.0 months, 7.5 months, 8.0 months, 8.5 months, 9.0 months, 9.5 months, 10 months, 10.5 months, 11 months, 11.5 months, 12 months, 13 months, 14 months, 15 months, 16 months, 17 months, 18 months, 19 months, 20 months, 21 months, 22 months, 23 months, 24 months, 25 months, 26 months, 27 months, 28 months, 29 months, 30 months, 31 months, 32 months, 33 months, 34 months, 35 months, or 36 months). In some instances, the treatment extends the OS of the subject by at least about 5.3 months (e.g., by 5.3-120, by 6-60 months, by 7-48 months, by 8-36 months, or by 10-24 months, e.g., by at least about 5.3 months, 5.5 months, 6.0 months, 6.5 months, 7.0 months, 7.5 months, 8.0 months, 8.5 months, 9.0 months, 9.5 months, 10 months, 10.5 months, 11 months, 11.5 months, 12 months, 13 months, 14 months, 15 months, 16 months, 17 months, 18 months, 19 months, 20 months, 21 months, 22 months, 23 months, 24 months, 25 months, 26 months, 27 months, 28 months, 29 months, 30 months, 31 months, 32 months, 33 months, 34 months, 35 months, or 36 months).

H. Therapeutic Methods and Uses Relating to Pancreatic Cancer

Pancreatic Cancer

Among pancreatic cancer patients, 80% present with advanced disease at initial diagnosis. Even patients who receive curative surgery will have disease relapses, resulting in 5-year survival rates of 25%-30% and 10% in patients with node-negative and node-positive disease at pancreaticoduodenectomy, respectively. Patients who have locally advanced and unresectable disease often receive radiochemotherapy, resulting in a median OS of 9-13 months, but rarely offering long-term survival.

Therefore, there is a high unmet need for improved medical intervention.

Methods and Uses for Treating Pancreatic Cancer

In some instances, a subject or population of subjects receiving the anti-TIGIT antagonist antibody (e.g., an anti-TIGIT antagonist antibody as disclosed herein, e.g., tiragolumab), the PD-1 axis binding antagonist (e.g., anti-PD-L1 antagonist antibody, such as atezolizumab), the antimetabolite (e.g., gemcitabine), and the taxane (e.g., paclitaxel) is being treated for a pancreatic cancer (e.g., a pancreatic ductal adenocarcinoma (PDAC), e.g., a metastatic PDAC (mPDAC))).

The present invention includes methods of treating a subject or a population of subjects having a pancreatic cancer, the method comprising administering to the subject or population of subjects a dosing regimen comprising one or more 28-day dosing cycles of tiragolumab at a dose of about 420 mg on Days 1 and 15 of each 28-day dosing cycle, atezolizumab at a dose of about 840 mg on Days 1 and 15 of each 28-day dosing cycle, gemcitabine at a dose of about 1000 mg/m² on Days 1, 8, and 15 of each 28-day dosing cycle, and nab-paclitaxel at a dose of about 125 mg/m² on Days 1, 8, and 15 of each 28-day dosing cycle. In some instances, the method comprises administering to the subject or population of subjects a dosing regimen comprising one or more 28-day dosing cycles of tiragolumab at a dose of 420 mg on Days 1 and 15 of each 28-day dosing cycle, atezolizumab at a dose of 840 mg on Days 1 and 15 of each 28-day dosing cycle, gemcitabine at a dose of 1000 mg/m² on Days 1, 8, and 15 of each 28-day dosing cycle, and nab-paclitaxel at a dose of 125 mg/m² on Days 1, 8, and 15 of each 28-day dosing cycle. In some instances, the pancreatic cancer is a PDAC, e.g., a mPDAC. In some aspects, the subject or subjects have not received prior systemic therapy for metastatic PDAC.

In some instances, the treatment results in an ORR of the population of subjects of at least about 41.7% to about 46.7% (e.g., 42%, 42.5%, 43%, 43.5%, 44%, 44.5%, 45%, 45.5%, 46%, 46.5%, or 46.7% (e.g., 41.7%-43%, 43%-45%, or 45%-46.7%). In some instances, the treatment results in an increase in ORR of at least about 20% compared to a treatment comprising gemcitabine and nab-paclitaxel without an anti-TIGIT antagonist antibody and a PD-1 axis binding antagonist. In some instances, the treatment results in a median PFS of the population of subjects of at least about 5.5 months (e.g., between about 5.5 months and 14 months (e.g., 6 months, 7 months, 8 months, 9 months, 10 months, 11 months, 12 months, 13 months, or 14 months)). In some instances, the treatment results in a median PFS of the population of subjects of at least about 7 months (e.g., between about 7 months and 14 months (e.g., 7 months, 8 months, 9 months, 10 months, 11 months, 12 months, 13 months, or 14 months)). In some instances, the treatment results in a median PFS of the population of subjects of at least about 5.5 months to about 7 months (e.g., 6 months, 6.2 months, 6.4 months, 6.6 months, 6.8 months, 7 months, or more than 7 months). In some instances, the treatment results in a median OS of the population of subjects of at least about 8.5 months (e.g., between about 8.5 months and about 16 months (8.5 months, 9 months, 9.5 months, 10 months, 10.5 months, 11 months, 12 months, 13 months, 14 months, 15 months, or 16 months)). In some instances, the treatment results in a median OS of the population of subjects of at least about 10.6 months (e.g., between about 10.6 months and about 16 months (10.6 months, 11 months, 12 months, 13 months, 14 months, 15 months, or 16 months)). In some instances, the treatment results in a median OS of the population of subjects of at least about 8.5 months to about 10.6 months (e.g., 8.7 months, 9.0 months, 9.2 months, 9.4 months, 9.6 months, 9.8 months, 10 months, 10.2 months, 10.4 months, 10.6 months, or more than 10.6 months).

Dosing of Agents

Dosing of anti-TIGIT antagonist antibodies, PD-1 axis binding antagonists, antimetabolites, and taxanes is described in Section III(K).

I. Therapeutic methods and uses relating to esophageal cancer

The present invention includes methods for treating a subject or population of subjects having an advanced or metastatic esophageal cancer, the method comprising administering to the subject or population of subjects a dosing regimen comprising one or more 21-day dosing cycles of an anti-TIGIT antagonist antibody at a dose (e.g., a fixed dose) of between about 30 mg to about 1200 mg (e.g., between about 550 mg to about 650 mg, e.g., 600 mg±10 mg, e.g., 600±6 mg, e.g., 600±5 mg, e.g., 600±3 mg, e.g., 600±1 mg, e.g., 600±0.5 mg, e.g., 600 mg) on Day 1 of each dosing cycle and a PD-1 axis binding antagonist at a dose (e.g., a fixed dose) of between about 80 mg to about 1600 mg (e.g., between about 1000 mg to about 1400 mg, e.g., between about 1050 mg to about 1350 mg, e.g., between about 1100 mg to about 1300 mg, e.g., between about 1150 mg to about 1250 mg, e.g., between about 1175 mg to about 1225 mg, e.g., between about 1190 mg to about 1210 mg, e.g., 1200 mg±5 mg, e.g., 1200±2.5 mg, e.g., 1200±1.0 mg, e.g., 1200±0.5 mg, e.g., 1200 mg) on Day 1 of each dosing cycle. In some instances, the method comprises administering to the subject or population of subjects a dosing regimen comprising one or more 21-day dosing cycles of an anti-TIGIT antagonist antibody at a dose (e.g., a fixed dose) of between about 500 mg to about 700 mg (e.g., between about 550 mg to about 650 mg, e.g., 600 mg±10 mg, e.g., 600±6 mg, e.g., 600±5 mg, e.g., 600±3 mg, e.g., 600±1 mg, e.g., 600±0.5 mg, e.g., 600 mg) on Day 1 of each dosing cycle and a PD-1 axis binding antagonist at a dose (e.g., a fixed dose) of between about 900 mg to about 1500 mg (e.g., between about 1000 mg to about 1400 mg, e.g., between about 1050 mg to about 1350 mg, e.g., between about 1100 mg to about 1300 mg, e.g., between about 1150 mg to about 1250 mg, e.g., between about 1175 mg to about 1225 mg, e.g., between about 1190 mg to about 1210 mg, e.g., 1200 mg±5 mg, e.g., 1200±2.5 mg, e.g., 1200±1.0 mg, e.g., 1200±0.5 mg, e.g., 1200 mg) on Day 1 of each dosing cycle. In some aspects, the method comprises administering to the subject or population of subjects a dosing regimen comprising one or more 21-day dosing cycles of an anti-TIGIT antagonist antibody at a dose (e.g., a fixed dose) of between 500 mg to 700 mg (e.g., between 550 mg to 650 mg, e.g., 600 mg±10 mg, e.g., 600±6 mg, e.g., 600±5 mg, e.g., 600±3 mg, e.g., 600±1 mg, e.g., 600±0.5 mg, e.g., 600 mg) on Day 1 of each dosing cycle and a PD-1 axis binding antagonist at a dose (e.g., a fixed dose) of between 900 mg to 1500 mg (e.g., between 1000 mg to 1400 mg, e.g., between 1050 mg to 1350 mg, e.g., between 1100 mg to 1300 mg, e.g., between 1150 mg to 1250 mg, e.g., between 1175 mg to 1225 mg, e.g., between 1190 mg to 1210 mg, e.g., 1200 mg±5 mg, e.g., 1200±2.5 mg, e.g., 1200±1.0 mg, e.g., 1200±0.5 mg, e.g., 1200 mg) on Day 1 of each dosing cycle.

The present invention includes methods for treating a subject or population of subjects having an esophageal cancer, the method comprising administering to the subject or population of subjects a dosing regimen comprising one or more 21-day dosing cycles of an anti-TIGIT antagonist antibody at a dose (e.g., a fixed dose) of between about 500 mg to about 700 mg (e.g., between about 550 mg to about 650 mg, e.g., 600 mg±10 mg, e.g., 600±6 mg, e.g., 600±5 mg, e.g., 600±3 mg, e.g., 600±1 mg, e.g., 600±0.5 mg, e.g., 600 mg) on Day 1 of each dosing cycle and a PD-1 axis binding antagonist at a dose (e.g., a fixed dose) of between about 900 mg to about 1500 mg (e.g., between about 1000 mg to about 1400 mg, e.g., between about 1050 mg to about 1350 mg, e.g., between about 1100 mg to about 1300 mg, e.g., between about 1150 mg to about 1250 mg, e.g., between about 1175 mg to about 1225 mg, e.g., between about 1190 mg to about 1210 mg, e.g., 1200 mg±5 mg, e.g., 1200±2.5 mg, e.g., 1200±1.0 mg, e.g., 1200±0.5 mg, e.g., 1200 mg) on Day 1 of each dosing cycle, wherein the subject or population of subjects has been previously treated with a platinum-based chemotherapeutic agent and a non-platinum-based chemotherapeutic agent. In some aspects, the method comprises administering to the subject or population of subjects a dosing regimen comprising one or more 21-day dosing cycles of an anti-TIGIT antagonist antibody at a dose (e.g., a fixed dose) of between 500 mg to 700 mg (e.g., between 550 mg to 650 mg, e.g., 600 mg±10 mg, e.g., 600±6 mg, e.g., 600±5 mg, e.g., 600±3 mg, e.g., 600±1 mg, e.g., 600±0.5 mg, e.g., 600 mg) on Day 1 of each dosing cycle and a PD-1 axis binding antagonist at a dose (e.g., a fixed dose) of between 900 mg to 1500 mg (e.g., between 1000 mg to 1400 mg, e.g., between 1050 mg to 1350 mg, e.g., between 1100 mg to 1300 mg, e.g., between 1150 mg to 1250 mg, e.g., between 1175 mg to 1225 mg, e.g., between 1190 mg to 1210 mg, e.g., 1200 mg±5 mg, e.g., 1200±2.5 mg, e.g., 1200±1.0 mg, e.g., 1200±0.5 mg, e.g., 1200 mg) on Day 1 of each dosing cycle, wherein the subject or population of subjects has been previously treated with a platinum-based chemotherapeutic agent and a non-platinum-based chemotherapeutic agent.

In some instances, the subject or population of subjects has been previously treated with a platinum-based chemotherapeutic agent and a non-platinum-based chemotherapeutic agent. In some instances, the subject or population of subjects has experienced disease progression or unacceptable toxicity during the previous treatment.

In some instances, the 21-day dosing cycles further comprise a platinum-based chemotherapeutic agent and a non-platinum-based chemotherapeutic agent. In some instances, the platinum-based chemotherapeutic agent is omitted from the dosing regimen after six doses.

In some instances, the platinum-based chemotherapeutic agent is cisplatin. In some instances, cisplatin is administered at a dose of about 80 mg/m² on Day 1 of each dosing cycle. In some instances, cisplatin is administered at a dose of 80 mg/m² on Day 1 of each dosing cycle.

In some instances, the non-platinum-based chemotherapeutic agent is an antimetabolite. In some instances, the antimetabolite is 5-fluorouracil. In some instances, 5-fluorouracil is administered at a dose of 800 mg/m²/24 hours on Days 1-5 of each 21-day cycle.

In some instances, the esophageal cancer is an advanced or metastatic esophageal cancer.

In some instances, the subject or subjects have had had no prior treatment for metastatic esophageal cancer.

The present invention includes methods for treating a subject or population of subjects having an advanced or metastatic esophageal cancer, the method comprising administering to the subject or population of subjects a dosing regimen comprising one or more 21-day dosing cycles of tiragolumab at a dose (e.g., a fixed dose) of about 600 mg on Day 1 of each dosing cycle, atezolizumab at a dose (e.g., a fixed dose) of about 1200 mg on Day 1 of each dosing cycle, cisplatin at a dose of about 80 mg/m² on Day 1 of each dosing cycle, and 5-fluorouracil at a dose of 800 mg/m²/24 hours on Days 1-5 of each 21-day cycle, wherein cisplatin is omitted from the dosing regimen after six doses. In some aspects, the method comprises administering to the subject or population of subjects a dosing regimen comprising one or more 21-day dosing cycles of tiragolumab at a dose (e.g., a fixed dose) of 600 mg on Day 1 of each dosing cycle, atezolizumab at a dose (e.g., a fixed dose) of 1200 mg on Day 1 of each dosing cycle, cisplatin at a dose of 80 mg/m² on Day 1 of each dosing cycle, and 5-fluorouracil at a dose of 800 mg/m²/24 hours on Days 1-5 of each 21-day cycle, wherein cisplatin is omitted from the dosing regimen after six doses.

In some instances, the effective amount of the anti-TIGIT antagonist antibody (e.g., an anti-TIGIT antagonist antibody as disclosed herein, e.g., tiragolumab) is a dose (e.g., a fixed dose) of between about 10 mg to about 1000 mg (e.g., between about 20 mg to about 1000 mg, e.g., between about 50 mg to about 900 mg, e.g., between about 100 mg to about 850 mg, e.g., between about 200 mg to about 800 mg, e.g., between about 300 mg to about 600 mg, e.g., between about 400 mg to about 500 mg, e.g., between about 405 mg to about 450 mg, e.g., between about 410 mg to about 430 mg, e.g., about 420 mg) every two weeks (Q2W). In some instances, the effective amount of the anti-TIGIT antagonist antibody (e.g., an anti-TIGIT antagonist antibody as disclosed herein, e.g., tiragolumab) is a dose (e.g., a fixed dose) of between about 10 mg to 1000 mg (e.g., between 20 mg to 1000 mg, e.g., between 50 mg to 900 mg, e.g., between 00 mg to 850 mg, e.g., between 200 mg to 800 mg, e.g., between 300 mg to 600 mg, e.g., between 400 mg to 500 mg, e.g., between 405 mg to 450 mg, e.g., between 410 mg to 430 mg, e.g., 420 mg) every two weeks (Q2W). In some instances, the effective amount of the anti-TIGIT antagonist antibody (e.g., an anti-TIGIT antagonist antibody as disclosed herein, e.g., tiragolumab) is a dose of about 420 mg every two weeks (e.g., 420 mg±10 mg, e.g., 420±6 mg, e.g., 420±5 mg, e.g., 420±3 mg, e.g., 420±1 mg, e.g., 420±0.5 mg, e.g., 420 mg every two weeks).

In some instances, the effective amount of the anti-TIGIT antagonist antibody (e.g., an anti-TIGIT antagonist antibody as disclosed herein, e.g., tiragolumab) is a dose of between about 200 mg to about 2000 mg (e.g., between about 200 mg to about 1600 mg, e.g., between about 250 mg to about 1600 mg, e.g., between about 300 mg to about 1600 mg, e.g., between about 400 mg to about 1500 mg, e.g., between about 500 mg to about 1400 mg, e.g., between about 600 mg to about 1200 mg, e.g., between about 700 mg to about 1100 mg, e.g., between about 800 mg to about 1000 mg, e.g., between about 800 mg to about 900 mg, e.g., about 800, about 810, about 820, about 830, about 840, about 850, about 860, about 870, about 880, about 890, or about 900 mg) every four weeks (Q4W). In some instances, the effective amount of the anti-TIGIT antagonist antibody (e.g., an anti-TIGIT antagonist antibody as disclosed herein, e.g., tiragolumab) is a dose of between 200 mg to 2000 mg (e.g., between 200 mg to 1600 mg, e.g., between 250 mg to 1600 mg, e.g., between 300 mg to 1600 mg, e.g., between 400 mg to 1500 mg, e.g., between 500 mg to 1400 mg, e.g., between 600 mg to 1200 mg, e.g., between 700 mg to 1100 mg, e.g., between 800 mg to 1000 mg, e.g., between 800 mg to 900 mg, e.g., 800, 810, 820, 830, 840, 850, 860, 870, 880, 890, or 900 mg) every four weeks (Q4W).

In some instances, the effective amount of the PD-1 axis binding antagonist (e.g., anti-PD-L1 antagonist antibody (e.g., atezolizumab)) is a dose of between about 80 mg to about 2000 mg (e.g., between about 80 mg to about 1950 mg, e.g., between about 80 mg to about 1900 mg, e.g., between about 80 mg to about 1800 mg, e.g., between about 100 mg to about 1700 mg, e.g., between about 200 mg to about 1600 mg, e.g., between about 300 mg to about 1400 mg, e.g., between about 400 mg to about 1300 mg, e.g., between about 500 mg to about 1200 mg, e.g., between about 600 mg to about 1100 mg, e.g., between about 700 mg to about 1000 mg, e.g., between about 740 mg to about 940 mg, e.g., between about 790 mg to about 890 mg, e.g., between about 815 mg to about 865 mg, e.g., between about 830 mg to about 850 mg, e.g., 840 mg±5 mg, e.g., 840±2.5 mg, e.g., 840±1.0 mg, e.g., 840±0.5 mg, e.g., 840 mg) every two weeks. In some instances, the effective amount of the PD-1 axis binding antagonist (e.g., anti-PD-L1 antagonist antibody (e.g., atezolizumab)) is a dose of between about 80 mg to about 2000 mg (e.g., between about 100 mg to about 2000 mg, e.g., between about 200 mg to about 2000 mg, e.g., between about 300 mg to about 2000 mg, e.g., between about 400 mg to about 2000 mg, e.g., between about 500 mg to about 2000 mg, e.g., between about 600 mg to about 1900 mg, e.g., between about 700 mg to about 1800 mg, e.g., between about 800 mg to about 1800 mg, e.g., between about 900 mg to about 1800 mg, e.g., between about 1000 mg to about 1800 mg, e.g., between about 1100 mg to about 1800 mg, e.g., between about 1200 mg to about 1800 mg, e.g., between about 1300 mg to about 1800 mg, e.g., between about 1400 mg to about 1800 mg, e.g., between about 1500 mg to about 1800 mg, e.g., between about 1580 mg to about 1780 mg, e.g., between about 1630 mg to about 1730 mg, e.g., between about 1655 mg to about 1705 mg, e.g., between about 1670 mg to about 1690 mg, e.g., 1680 mg±5 mg, e.g., 1680±2.5 mg, e.g., 1680±1.0 mg, e.g., 1680±0.5 mg, e.g., 1680 mg) every four weeks. In some instances, the effective amount of the PD-1 axis binding antagonist (e.g., anti-PD-L1 antagonist antibody (e.g., atezolizumab)) is a dose of between 80 mg to 2000 mg (e.g., between 80 mg to 1950 mg, e.g., between 80 mg to 1900 mg, e.g., between 80 mg to 1800 mg, e.g., between 100 mg to 1700 mg, e.g., between 200 mg to 1600 mg, e.g., between 300 mg to 1400 mg, e.g., between 400 mg to 1300 mg, e.g., between 500 mg to 1200 mg, e.g., between 600 mg to 1100 mg, e.g., between 700 mg to 1000 mg, e.g., between 740 mg to 940 mg, e.g., between 790 mg to 890 mg, e.g., between 815 mg to 865 mg, e.g., between 830 mg to 850 mg, e.g., 840 mg±5 mg, e.g., 840±2.5 mg, e.g., 840±1.0 mg, e.g., 840±0.5 mg, e.g., 840 mg) every two weeks. In some instances, the effective amount of the PD-1 axis binding antagonist (e.g., anti-PD-L1 antagonist antibody (e.g., atezolizumab)) is a dose of between 80 mg to 2000 mg (e.g., between 100 mg to 2000 mg, e.g., between 200 mg to 2000 mg, e.g., between 300 mg to 2000 mg, e.g., between 400 mg to 2000 mg, e.g., between 500 mg to 2000 mg, e.g., between 600 mg to 1900 mg, e.g., between 700 mg to 1800 mg, e.g., between 800 mg to 1800 mg, e.g., between 900 mg to 1800 mg, e.g., between 1000 mg to 1800 mg, e.g., between 1100 mg to 1800 mg, e.g., between 1200 mg to 1800 mg, e.g., between 1300 mg to 1800 mg, e.g., between 1400 mg to 1800 mg, e.g., between 1500 mg to 1800 mg, e.g., between 1580 mg to 1780 mg, e.g., between 1630 mg to 1730 mg, e.g., between 1655 mg to 1705 mg, e.g., between 1670 mg to 1690 mg, e.g., 1680 mg±5 mg, e.g., 1680±2.5 mg, e.g., 1680 1.0 mg, e.g., 1680±0.5 mg, e.g., 1680 mg) every four weeks. In some instances, the effective amount of the PD-1 axis binding antagonist (e.g., anti-PD-L1 antagonist antibody (e.g., atezolizumab)) is a dose of about 840 mg every two weeks. In some instances, the effective amount of the PD-1 axis binding antagonist (e.g., anti-PD-L1 antagonist antibody (e.g., atezolizumab)) is a dose of 840 mg every two weeks. In some instances, the effective amount of the PD-1 axis binding antagonist (e.g., anti-PD-L1 antagonist antibody (e.g., atezolizumab)) is a dose of about 1680 mg every four weeks. In some instances, the effective amount of the PD-1 axis binding antagonist (e.g., anti-PD-L1 antagonist antibody (e.g., atezolizumab)) is a dose of 1680 mg every four weeks. In some instances, the dose of the PD-1 axis binding antagonist (e.g., anti-PD-L1 antagonist antibody (e.g., atezolizumab)) administered in a combination therapy (e.g., a combination treatment with an anti-TIGIT antagonist antibody, such as an anti-TIGIT antagonist antibody disclosed herein, e.g., tiragolumab) may be reduced as compared to a standard dose of the anti-PD-L1 antagonist antibody administered as a monotherapy.

The present invention includes methods for treating a subject or population of subjects having an advanced or metastatic esophageal cancer, the method comprising administering to the subject or population of subjects a first dosing regimen and a second dosing regimen, wherein (a) the first dosing regimen comprises one or more 21-day dosing cycles of cisplatin at a dose of about 80 mg/m² on Day 1 of each dosing cycle and 5-fluorouracil at a dose of 800 mg/m²/24 hours on Days 1-5 of each 21-day cycle, wherein cisplatin is omitted from the dosing regimen after six doses; and (b) the second dosing regimen comprises one or more 21-day dosing cycles of tiragolumab at a dose of about 600 mg on Day 1 of each dosing cycle and atezolizumab at a dose of about 1200 mg on Day 1 of each dosing cycle.

In some aspects of any of the above methods, the treatment results in an ORR of the population of subjects of at least about 14% (e.g., results in an ORR of at least about 14%, 15%, 16%, 17%, 18%, 19%, 20%, 25%, 30%, 35%, 40%, 45%, 50%, 55%, 60%, 65%, 70%, 75%, 80%, 85%, 90%, 95%, or 100%, e.g., 14%-16%, 16%-18%, 18%-20%, 20%-30%, 30%-40%, 40%-50%, 50%-60%, 60%-70%, 70%-80%, 80%-90%, or 90%-100%).

J. Diagnostic Methods and Uses Relating to Cancer

The invention provides methods for selecting a therapy for a subject having a cancer (e.g., a lung cancer (e.g., an early stage lung cancer (e.g., a resectable lung cancer), a SCLC (e.g., an ES-SCLC), a NSCLC (e.g., a squamous NSCLC or a non-squamous NSCLC, a locally advanced unresectable NSCLC, a Stage IIIB NSCLC, a recurrent or metastatic NSCLC (e.g., a locally advanced unresectable or metastatic non-squamous NSCLC (e.g., Stage IV non-squamous NSCLC)), or a Stage IV NSCLC (e.g., wherein the subject has not been previously treated for Stage IV NSCLC))); a cervical cancer (e.g., a Stage IVB, metastatic, recurrent, or persistent cervical cancer, e.g., a metastatic and/or recurrent PD-L1-positive cervical carcinoma); a breast cancer (e.g., a TNBC (e.g., an eTNBC)) or a HER2-positive breast cancer); a head and neck cancer (e.g., SCCHN, e.g., recurrent/metastatic PD-L1-positive SCCHN); a liver cancer (e.g., HCC, e.g., locally advanced or metastatic HCC and/or unresectable HCC); a bladder cancer (e.g., MIBC, locally advanced UC, or mUC); an esophageal cancer; a pancreatic cancer (e.g., PDAC, e.g., metastatic PDAC); a kidney or renal cancer (e.g., a RCC); a melanoma; an ovarian cancer; a gastric cancer (e.g., a gastroesophageal junction cancer); or a CRC (e.g., MSS or MSI-Low CRC)), wherein therapy is guided by diagnostic methods that involve determining the presence and/or expression levels/amount of one or more biomarkers (e.g., PD-L1, TIGIT, activated T cells, or cytokines) in a sample (e.g., a tumor sample or a blood sample) obtained from the subject.

Additionally provided herein are methods for identifying a subject having a cancer (e.g., an early stage lung cancer (e.g., a resectable lung cancer), a SCLC (e.g., an ES-SCLC), a NSCLC (e.g., a squamous NSCLC or a non-squamous NSCLC, a locally advanced unresectable NSCLC, a Stage IIIB NSCLC, a recurrent or metastatic NSCLC (e.g., a locally advanced unresectable or metastatic non-squamous NSCLC (e.g., Stage IV non-squamous NSCLC)), or a Stage IV NSCLC (e.g., wherein the subject has not been previously treated for Stage IV NSCLC))); a cervical cancer (e.g., a Stage IVB, metastatic, recurrent, or persistent cervical cancer, e.g., a metastatic and/or recurrent PD-L1-positive cervical carcinoma); a breast cancer (e.g., a TNBC (e.g., an eTNBC)) or a HER2-positive breast cancer); a head and neck cancer (e.g., SCCHN, e.g., recurrent/metastatic PD-L1-positive SCCHN); a liver cancer (e.g., HCC, e.g., locally advanced or metastatic HCC and/or unresectable HCC); a bladder cancer (e.g., MIBC, locally advanced UC, or mUC); an esophageal cancer; a pancreatic cancer (e.g., PDAC, e.g., metastatic PDAC); a kidney or renal cancer (e.g., a RCC); a melanoma; an ovarian cancer; a gastric cancer (e.g., a gastroesophageal junction cancer); or a CRC (e.g., MSS or MSI-Low CRC)) who may benefit from a treatment comprising an anti-TIGIT antagonist antibody (e.g., an anti-TIGIT antagonist antibody as disclosed herein, e.g., tiragolumab) and a PD-1 axis binding antagonist (e.g., an anti-PD-L1 antagonist antibody, such as atezolizumab, or an anti-PD-1 antagonist antibody such as pembrolizumab), wherein identification is guided by diagnostic methods that involve determining the presence and/or expression levels/amount of one or more biomarkers (e.g., PD-L1, TIGIT, activated T cells, or cytokines) in a sample (e.g., a tumor sample or a blood sample) obtained from the subject.

Additionally provided herein are methods for assessing responsiveness to a therapy for a subject having a cancer (e.g., an early stage lung cancer (e.g., a resectable lung cancer), a SCLC (e.g., an ES-SCLC), a NSCLC (e.g., a squamous NSCLC or a non-squamous NSCLC, a locally advanced unresectable NSCLC, a Stage IIIB NSCLC, a recurrent or metastatic NSCLC (e.g., a locally advanced unresectable or metastatic non-squamous NSCLC (e.g., Stage IV non-squamous NSCLC)), or a Stage IV NSCLC (e.g., wherein the subject has not been previously treated for Stage IV NSCLC))); a cervical cancer (e.g., a Stage IVB, metastatic, recurrent, or persistent cervical cancer, e.g., a metastatic and/or recurrent PD-L1-positive cervical carcinoma); a breast cancer (e.g., a TNBC (e.g., an eTNBC)) or a HER2-positive breast cancer); a head and neck cancer (e.g., SCCHN, e.g., recurrent/metastatic PD-L1-positive SCCHN); a liver cancer (e.g., HCC, e.g., locally advanced or metastatic HCC and/or unresectable HCC); a bladder cancer (e.g., MIBC, locally advanced UC, or mUC); an esophageal cancer; a pancreatic cancer (e.g., PDAC, e.g., metastatic PDAC); a kidney or renal cancer (e.g., a RCC); a melanoma; an ovarian cancer; a gastric cancer (e.g., a gastroesophageal junction cancer); or a CRC (e.g., MSS or MSI-Low CRC)), wherein further therapy is guided by diagnostic methods that involve determining the presence and/or expression levels/amount of one or more biomarkers (e.g., PD-L1, TIGIT, activated T cells, or cytokines) in a sample (e.g., a tumor sample or a blood sample) obtained from the subject.

Additionally provided herein are methods for optimizing a therapy for a subject having a cancer (e.g., an early stage lung cancer (e.g., a resectable lung cancer), a SCLC (e.g., an ES-SCLC), a NSCLC (e.g., a squamous NSCLC or a non-squamous NSCLC, a locally advanced unresectable NSCLC, a Stage IIIB NSCLC, a recurrent or metastatic NSCLC (e.g., a locally advanced unresectable or metastatic non-squamous NSCLC (e.g., Stage IV non-squamous NSCLC)), or a Stage IV NSCLC (e.g., wherein the subject has not been previously treated for Stage IV NSCLC))); a cervical cancer (e.g., a Stage IVB, metastatic, recurrent, or persistent cervical cancer, e.g., a metastatic and/or recurrent PD-L1-positive cervical carcinoma); a breast cancer (e.g., a TNBC (e.g., an eTNBC)) or a HER2-positive breast cancer); a head and neck cancer (e.g., SCCHN, e.g., recurrent/metastatic PD-L1-positive SCCHN); a liver cancer (e.g., HCC, e.g., locally advanced or metastatic HCC and/or unresectable HCC); a bladder cancer (e.g., MIBC, locally advanced UC, or mUC); an esophageal cancer; a pancreatic cancer (e.g., PDAC, e.g., metastatic PDAC); a kidney or renal cancer (e.g., a RCC); a melanoma; an ovarian cancer; a gastric cancer (e.g., a gastroesophageal junction cancer); or a CRC (e.g., MSS or MSI-Low CRC)), wherein further therapy is guided by diagnostic methods that involve determining the presence and/or expression levels/amount of one or more biomarkers (e.g., PD-L1, TIGIT, activated T cells, or cytokines) in a sample (e.g., a tumor sample or a blood sample) obtained from the subject.

Biomarkers for use in the methods described herein can include, but are not limited to, PD-L1 and/or TIGIT expression on tissues (e.g., tumor tissues) or in blood (e.g., whole blood), germline and somatic mutations from tissue (e.g., tumor tissue) and/or from circulating tumor DNA in blood (including, but not limited to, mutation load, MSI, and MMR defects), identified through WGS and/or NGS, analysis of genes (e.g., CD274) or gene signatures associated with tumor immunobiology (e.g., TEFF), lymphocyte subpopulations, T cell-receptor repertoire, cytokines associated with T-cell activation, and plasma derived cytokines. In some instances, the biomarker is PD-L1. In some instances, the sample is a tumor sample (e.g., a formalin-fixed, paraffin-embedded (FFPE) tumor sample).

In some instances, the method includes determining the presence and/or expression levels/amount of a biomarker (e.g., PD-L1, TIGIT, activated T cells, or cytokines) in a sample (e.g., a tumor sample or a blood sample) from the subject, and administering to the subject one or more dosing cycles of an effective amount of an anti-TIGIT antagonist antibody (e.g., an anti-TIGIT antagonist antibody as disclosed herein, e.g., tiragolumab) and a PD-1 axis binding antagonist (e.g., an anti-PD-L1 antagonist antibody, such as atezolizumab, or an anti-PD-1 antagonist antibody, such as e.g., pembrolizumab) to a subject in accordance with any of the methods or uses described in Section III(A).

In some instances, the method includes determining the presence and/or expression levels/amount of a biomarker (e.g., PD-L1, TIGIT, activated T cells, or cytokines) in a sample (e.g., a tumor sample or a blood sample) from the subject, and administering to the subject a dosing regimen comprising one or more dosing cycles of an anti-TIGIT antagonist antibody at a dose (e.g., a fixed dose) of about 700 mg to about 1000 mg every four weeks and a PD-1 axis binding antagonist at a dose (e.g., a fixed dose) of about 1400 mg to 2000 mg every four weeks. In some instances, the dosing regimen comprises one or more dosing cycles of an anti-TIGIT antagonist antibody at a dose (e.g., a fixed dose) of 700 mg to 1000 mg every four weeks and a PD-1 axis binding antagonist at a dose (e.g., a fixed dose) of 1400 mg to 2000 mg every four weeks.

In some instances, the method includes determining the presence and/or expression levels/amount of a biomarker (e.g., PD-L1, TIGIT, activated T cells, or cytokines) in a sample (e.g., a tumor sample or a blood sample) from the subject, and administering to the subject a dosing regimen comprising one or more dosing cycles of an anti-TIGIT antagonist antibody at a dose (e.g., a fixed dose) of about 300 mg to about 600 mg every two weeks and a PD-1 axis binding antagonist at a dose (e.g., a fixed dose) of about 600 mg to about 1200 mg every two weeks. In some instances, the method comprises administering to the subject a dosing regimen comprising one or more dosing cycles of an anti-TIGIT antagonist antibody at a dose (e.g., a fixed dose) of 300 mg to 600 mg every two weeks and a PD-1 axis binding antagonist at a dose (e.g., a fixed dose) of 600 mg to 1200 mg every two weeks.

In some instances, the method includes determining the presence and/or expression levels/amount of a biomarker (e.g., PD-L1, TIGIT, activated T cells, or cytokines) in a sample (e.g., a tumor sample or a blood sample) from the subject, and administering to the subject a dosing regimen comprising one or more dosing cycles of an anti-TIGIT antagonist antibody at a dose (e.g., a fixed dose) from about 30 mg to about 1200 mg every three weeks, a PD-1 axis binding antagonist at a dose (e.g., a fixed dose) from about 80 and 1600 mg every three weeks, a platinum-based chemotherapeutic agent every three weeks, and a non-platinum-based chemotherapeutic agent every three weeks. In some instances, the method comprises administering to the subject a dosing regimen comprising one or more dosing cycles of an anti-TIGIT antagonist antibody at a dose (e.g., a fixed dose) from 30 mg to 1200 mg every three weeks, a PD-1 axis binding antagonist at a dose (e.g., a fixed dose) from 80 and 1600 mg every three weeks, a platinum-based chemotherapeutic agent every three weeks, and a non-platinum-based chemotherapeutic agent every three weeks.

In some instances, the method includes determining the presence and/or expression levels/amount of a biomarker (e.g., PD-L1, TIGIT, activated T cells, or cytokines) in a sample (e.g., a tumor sample or a blood sample) from the subject, and administering to the subject a dosing regimen comprising one or more dosing cycles of an anti-TIGIT antagonist antibody at a dose from about 30 mg to about 1200 mg every three weeks and an anti-PD-1 antagonist antibody at a dose of about 200 mg every three weeks, wherein the anti-PD-1 antagonist antibody is pembrolizumab. In some instances, the method comprises administering to the subject a dosing regimen comprising one or more dosing cycles of an anti-TIGIT antagonist antibody at a dose from 30 mg to 1200 mg every three weeks and an anti-PD-1 antagonist antibody at a dose of 200 mg every three weeks, wherein the anti-PD-1 antagonist antibody is pembrolizumab.

In some instances, the method includes determining the presence and/or expression levels/amount of a biomarker (e.g., PD-L1, TIGIT, activated T cells, or cytokines) in a sample (e.g., a tumor sample or a blood sample) from the subject, and administering to the subject a dosing regimen comprising one or more dosing cycles of tiragolumab and pembrolizumab, wherein the pembrolizumab is administered at a dose between about 100 mg to about 1000 mg every six weeks. In some instances, the pembrolizumab is administered at a dose of about 400 mg every six weeks.

In some instances, the method includes determining the presence and/or expression levels/amount of a biomarker (e.g., PD-L1, TIGIT, activated T cells, or cytokines) in a sample (e.g., a tumor sample or a blood sample) from the subject, and administering to the subject a dosing regimen comprising one or more dosing cycles of an anti-TIGIT antagonist antibody at a dose (e.g., a fixed dose) of between about 30 mg to about 1200 mg every three weeks, a PD-1 axis binding antagonist at a dose (e.g., a fixed dose) of between about 80 mg to about 1600 mg every three weeks, and an antimetabolite at a dose (e.g., a fixed dose) of between about 10 mg/m² to about 10000 mg/m² twice a day orally every three weeks for 2-weeks on/1-week off. In some instances, the method includes administering to the subject a dosing regimen comprising one or more dosing cycles of an anti-TIGIT antagonist antibody at a dose (e.g., a fixed dose) of between 30 mg to 1200 mg every three weeks, a PD-1 axis binding antagonist at a dose (e.g., a fixed dose) of between 80 mg to 1600 mg every three weeks, and an antimetabolite at a dose (e.g., a fixed dose) of between 10 mg/m² to 10000 mg/m² twice a day orally every three weeks for 2-weeks on/1-week off.

In some instances, the method includes determining the presence and/or expression levels/amount of a biomarker (e.g., PD-L1, TIGIT, activated T cells, or cytokines) in a sample (e.g., a tumor sample or a blood sample) from the subject, and administering to the subject a dosing regimen comprising one or more dosing cycles of an anti-TIGIT antagonist antibody at a dose (e.g., a fixed dose) of about 30 mg to about 1200 mg every three weeks, a PD-1 axis binding antagonist at a dose (e.g., a fixed dose) of about 80 and 1600 mg every three weeks, gemcitabine, and nab-paclitaxel. In some instances, the method includes administering to the subject a dosing regimen comprising one or more dosing cycles of an anti-TIGIT antagonist antibody at a dose (e.g., a fixed dose) of 30 mg to 1200 mg every three weeks, a PD-1 axis binding antagonist at a dose (e.g., a fixed dose) of 80 and 1600 mg every three weeks, gemcitabine, and nab-paclitaxel.

In some instances, the method includes determining the presence and/or expression levels/amount of a biomarker (e.g., PD-L1, TIGIT, activated T cells, or cytokines) in a sample (e.g., a tumor sample or a blood sample) from the subject, and administering to the subject a dosing regimen comprising one or more dosing cycles of an anti-TIGIT antagonist antibody at a dose (e.g., a fixed dose) of between about 30 mg to about 1200 mg every three weeks, a PD-1 axis binding antagonist at a dose (e.g., a fixed dose) of between about 80 mg to about 1600 mg every three weeks, and a VEGF antagonist at a dose of between about 1 mg/kg to about 35 mg/kg every three weeks. In some instances, the method includes administering to the subject a dosing regimen comprising one or more dosing cycles of an anti-TIGIT antagonist antibody at a dose (e.g., a fixed dose) of between 30 mg to 1200 mg every three weeks, a PD-1 axis binding antagonist at a dose (e.g., a fixed dose) of between 80 mg to 1600 mg every three weeks, and a VEGF antagonist at a dose of between 1 mg/kg to 35 mg/kg every three weeks.

In some instances, the method includes determining the presence and/or expression levels/amount of a biomarker (e.g., PD-L1, TIGIT, activated T cells, or cytokines) in a sample (e.g., a tumor sample or a blood sample) from the subject, and administering to the subject a dosing regimen comprising an induction phase and a maintenance phase, wherein: (a) the induction phase comprises one or more dosing cycles of an anti-TIGIT antagonist antibody at a dose (e.g., a fixed dose) from about 30 mg to about 1200 mg every three weeks, a PD-1 axis binding antagonist at a dose (e.g., a fixed dose) from about 80 and 1600 mg every three weeks, a platinum-based chemotherapeutic agent every three weeks, and a non-platinum-based chemotherapeutic agent every three weeks; and (b) the maintenance phase comprises one or more additional dosing cycles of the anti-TIGIT antagonist antibody every three weeks, the PD-1 axis binding antagonist every three weeks, and the non-platinum-based chemotherapeutic agent every three weeks, and wherein the maintenance phase does not comprise administration of the platinum-based chemotherapeutic agent. In some instances, (a) the induction phase comprises one or more dosing cycles of an anti-TIGIT antagonist antibody at a dose (e.g., a fixed dose) from 30 mg to 1200 mg every three weeks, a PD-1 axis binding antagonist at a dose (e.g., a fixed dose) from 80 and 1600 mg every three weeks, a platinum-based chemotherapeutic agent every three weeks, and a non-platinum-based chemotherapeutic agent every three weeks; and (b) the maintenance phase comprises one or more additional dosing cycles of the anti-TIGIT antagonist antibody every three weeks, the PD-1 axis binding antagonist every three weeks, and the non-platinum-based chemotherapeutic agent every three weeks, and wherein the maintenance phase does not comprise administration of the platinum-based chemotherapeutic agent.

In some instances, the method includes determining the presence and/or expression levels/amount of a biomarker (e.g., PD-L1, TIGIT, activated T cells, or cytokines) in a sample (e.g., a tumor sample or a blood sample) from the subject, and administering to the subject a dosing regimen comprising an induction phase and a maintenance phase, wherein: (a) the induction phase comprises one or more dosing cycles of an anti-TIGIT antagonist antibody at a dose (e.g., a fixed dose) from about 500 mg to about 700 mg every three weeks, a PD-1 axis binding antagonist at a dose (e.g., a fixed dose) from about 900 mg to about 1500 mg every three weeks, a platinum-based chemotherapeutic agent every three weeks, and a non-platinum-based chemotherapeutic agent every three weeks; and (b) the maintenance phase comprises one or more additional dosing cycles of the anti-TIGIT antagonist antibody at a dose (e.g., a fixed dose) of about 700 mg to about 1000 mg every four weeks and the PD-1 axis binding antagonist at a dose (e.g., a fixed dose) of about 1400 mg to 2000 mg every four weeks, wherein the maintenance phase does not comprise administration of the platinum-based chemotherapeutic agent or non-platinum-based chemotherapeutic agent. In some instances, (a) the induction phase comprises one or more dosing cycles of an anti-TIGIT antagonist antibody at a dose (e.g., a fixed dose) from 500 mg to 700 mg every three weeks, a PD-1 axis binding antagonist at a dose (e.g., a fixed dose) from 900 mg to 1500 mg every three weeks, a platinum-based chemotherapeutic agent every three weeks, and a non-platinum-based chemotherapeutic agent every three weeks; and (b) the maintenance phase comprises one or more additional dosing cycles of the anti-TIGIT antagonist antibody at a dose (e.g., a fixed dose) of 700 mg to 1000 mg every four weeks and the PD-1 axis binding antagonist at a dose (e.g., a fixed dose) of 1400 mg to 2000 mg every four weeks, wherein the maintenance phase does not comprise administration of the platinum-based chemotherapeutic agent or non-platinum-based chemotherapeutic agent.

Presence and/or expression levels/amount of a biomarker (e.g., PD-L1, TIGIT, activated T cells, or cytokines) can be determined qualitatively and/or quantitatively based on any suitable criterion known in the art, including but not limited to proteins, protein fragments, DNA, mRNA, cDNA, and/or gene copy number. In some instances, the biomarker is PD-L1. PD-L1 expression may be assessed as described in Section III(L). In some instances, the biomarker is TIGIT. TIGIT expression may be assessed as described in Section III(M). In some instances, the biomarker is an EGFR and/or ALK aberration. EGFR and/or ALK aberrations may be assessed as described in Section III(N).

In some instances, expression levels or amount of a biomarker is a detectable protein expression level of PD-L1 in a tumor sample (e.g., a FFPE tumor sample) from the subject. In some instances, the PD-L1 protein expression level has been determined by an immunohistochemical (IHC) assay. In some instances, the tumor sample is a FFPE tumor sample.

In some instances, the tumor sample (e.g., FFPE tumor sample) from the subject has been determined to have a detectable expression level of PD-L1. In some instances, the tumor sample (e.g., FFPE tumor sample) from the subject has been determined to have a detectable expression level of PD-L1 in tumor-infiltrating immune cells. In some instances, the tumor sample is a FFPE tumor sample.

In some instances, the expression levels or amount of a biomarker is a detectable nucleic acid expression level of PD-L1 in a tumor sample (e.g., FFPE tumor sample) from the subject. In some instances, the PD-L1 nucleic acid expression level has been determined by RNA-seq, RT-qPCR, qPCR, multiplex qPCR, or RT-qPCR, microarray analysis, serial analysis of gene expression (SAGE), MassARRAY® technique, in situ hybridization (ISH), or a combination thereof. In some instances, the tumor sample is a FFPE tumor sample.

In some instances, the presence and/or expression levels/amount of the biomarker (e.g., PD-L1, TIGIT, activated T cells, or cytokines) in a sample (e.g., a tumor sample or a blood sample) from a subject selects the subject as eligible for therapy with an anti-TIGIT antagonist antibody and a PD-1 axis binding antagonist (e.g., an anti-PD-L1 antagonist antibody, such as atezolizumab, or an anti-PD-1 antagonist antibody such as pembrolizumab), for example, where a detectable expression level of PD-L1 is a biomarker for selection of individuals. In some instances, the sample is selected from the group consisting of a tissue sample, a whole blood sample, a serum sample, and a plasma sample. In some instances, the tissue sample is a tumor sample (e.g., FFPE tumor sample). In some instances, the tumor sample comprises tumor-infiltrating immune cells, tumor cells, stromal cells, and any combinations thereof. In some instances, the tumor sample is a FFPE tumor sample.

In some instances, the method further includes administering to the identified subject the therapy. In some instances, the therapy may further include, or be administered in conjunction with (either separately or together), one or more additional therapeutic agent(s) (e.g., an anti-TIGIT antagonist antibody, a PD-1 axis binding antagonist (e.g., an anti-PD-L1 antibody), a VEGF antagonist, a chemotherapeutic agent (e.g., a platinum-based chemotherapeutic agent or non-platinum-based chemotherapeutic agent), an ADC (e.g., enfortumab vedotin or sacituzumab govitecan), or a CSF (e.g., pegfilgrastim, filgrastim, or sargramostim)).

In some instances, in any of the diagnostic methods or uses described herein, the cancer is a solid tumor and/or a locally advanced or metastatic cancer.

K. Dosing

i. Dosing of Anti-TIGIT Antagonist Antibodies

As a general proposition, the therapeutically effective amount of an anti-TIGIT antagonist antibody (e.g., an anti-TIGIT antagonist antibody as disclosed herein, e.g., tiragolumab) administered to a human will be in the range of about 0.01 to about 50 mg/kg of patient body weight, whether by one or more administrations. In some embodiments, the therapeutically effective amount of an anti-TIGIT antagonist antibody (e.g., an anti-TIGIT antagonist antibody as disclosed herein, e.g., tiragolumab) administered to a human is in the range of 0.01 to 50 mg/kg of patient body weight, whether by one or more administrations.

In some exemplary embodiments, the anti-TIGIT antagonist antibody (e.g., an anti-TIGIT antagonist antibody as disclosed herein, e.g., tiragolumab) is administered in a dose of about 0.01 to about 45 mg/kg, about 0.01 to about 40 mg/kg, about 0.01 to about 35 mg/kg, about 0.01 to about 30 mg/kg, about 0.01 to about 25 mg/kg, about 0.01 to about 20 mg/kg, about 0.01 to about 15 mg/kg, about 0.01 to about 10 mg/kg, about 0.01 to about 5 mg/kg, or about 0.01 to about 1 mg/kg administered daily, weekly, every two weeks, every three weeks, or every four weeks, for example. In exemplary embodiments, the anti-TIGIT antagonist antibody (e.g., an anti-TIGIT antagonist antibody as disclosed herein, e.g., tiragolumab) is administered in a dose of 0.01 to 45 mg/kg, 0.01 to 40 mg/kg, 0.01 to 35 mg/kg, 0.01 to 30 mg/kg, 0.01 to 25 mg/kg, 0.01 to 20 mg/kg, 0.01 to 15 mg/kg, 0.01 to 10 mg/kg, 0.01 to 5 mg/kg, or 0.01 to 1 mg/kg administered daily, weekly, every two weeks, every three weeks, or every four weeks, for example.

In some instances, the anti-TIGIT antagonist antibody (e.g., an anti-TIGIT antagonist antibody as disclosed herein, e.g., tiragolumab) is administered on about Day 1 (e.g., Day −3, Day −2, Day −1, Day 1, Day 2, or Day 3) of a dosing cycle.

In some instances, the anti-TIGIT antagonist antibody (e.g., an anti-TIGIT antagonist antibody as disclosed herein, e.g., tiragolumab) is administered (e.g., every three weeks) in a tiered dosing regimen (e.g., dosing based on body weight (BW) or body surface area (BSA) of a subject). Such dosing regimens can be utilized in treatments for subjects having relatively low body weight (e.g., 40 kg or less (e.g., from 5 kg to 40 kg, from 15 kg to 40 kg, or from 5 kg to 15 kg)) and have been developed through biosimulation studies based on extrapolations of pharmacokinetic parameters estimated from adult data.

In some instances, the effective amount of the anti-TIGIT antagonist antibody (e.g., an anti-TIGIT antagonist antibody as disclosed herein, e.g., tiragolumab) to treat a subject having a cancer is a tiered dose based on a subject's body weight. In some instances, the effective amount of the anti-TIGIT antagonist antibody (e.g., an anti-TIGIT antagonist antibody as disclosed herein, e.g., tiragolumab) is a tiered dose based on a subject's body weight, wherein the subject has a body weight of (a) less than or equal to 15 kg, and the anti-TIGIT antagonist antibody is administered at a dose of between about 10 mg to about 1000 mg every three weeks (e.g., about 300 mg every three weeks); (b) greater than 15 kg and less than or equal to 40 kg, and the anti-TIGIT antagonist antibody is administered at a dose of between about 10 mg to about 1000 mg every three weeks (e.g., about 400 mg every three weeks); or (c) greater than 40 kg, and the anti-TIGIT antagonist antibody is administered at a dose of between about 30 mg to about 1200 mg every three weeks (e.g., about 600 mg every three weeks). In some instances, the effective amount of the anti-TIGIT antagonist antibody (e.g., an anti-TIGIT antagonist antibody as disclosed herein, e.g., tiragolumab) is a tiered dose based on a subject's body weight, wherein the subject has a body weight of (a) less than or equal to 15 kg, and the anti-TIGIT antagonist antibody is administered at a dose of between about 250 mg to about 350 mg every three weeks (e.g., about 300 mg every three weeks); (b) greater than 15 kg and less than or equal to 40 kg, and the anti-TIGIT antagonist antibody is administered at a dose of between about 350 mg to about 450 mg every three weeks (e.g., about 400 mg every three weeks); or (c) greater than 40 kg, and the anti-TIGIT antagonist antibody is administered at a dose of between about 550 mg to about 650 mg every three weeks (e.g., about 600 mg every three weeks). In some instances, the effective amount of the anti-TIGIT antagonist antibody (e.g., an anti-TIGIT antagonist antibody as disclosed herein, e.g., tiragolumab) is a tiered dose based on a subject's body weight, wherein the subject has a body weight of (a) less than or equal to 15 kg, and the anti-TIGIT antagonist antibody is administered at a dose of about 300 mg every three weeks; (b) greater than 15 kg and less than or equal to 40 kg, and the anti-TIGIT antagonist antibody is administered at a dose of about 400 mg every three weeks; or (c) greater than 40 kg, and the anti-TIGIT antagonist antibody is administered at a dose of about 600 mg every three weeks. In some instances, the effective amount of the anti-TIGIT antagonist antibody (e.g., an anti-TIGIT antagonist antibody as disclosed herein, e.g., tiragolumab) is a tiered dose based on a subject's body weight, wherein the subject has a body weight of (a) less than or equal to 15 kg, and the anti-TIGIT antagonist antibody is administered at a dose of between 10 mg to 1000 mg every three weeks (e.g., 300 mg every three weeks); (b) greater than 15 kg and less than or equal to 40 kg, and the anti-TIGIT antagonist antibody is administered at a dose of between 10 mg to 1000 mg every three weeks (e.g., 400 mg every three weeks); or (c) greater than 40 kg, and the anti-TIGIT antagonist antibody is administered at a dose of between 30 mg to 1200 mg every three weeks (e.g., 600 mg every three weeks). In some instances, the effective amount of the anti-TIGIT antagonist antibody (e.g., an anti-TIGIT antagonist antibody as disclosed herein, e.g., tiragolumab) is a tiered dose based on a subject's body weight, wherein the subject has a body weight of (a) less than or equal to 15 kg, and the anti-TIGIT antagonist antibody is administered at a dose of between 250 mg to 350 mg every three weeks (e.g., 300 mg every three weeks); (b) greater than 15 kg and less than or equal to 40 kg, and the anti-TIGIT antagonist antibody is administered at a dose of between 350 mg to 450 mg every three weeks (e.g., 400 mg every three weeks); or (c) greater than 40 kg, and the anti-TIGIT antagonist antibody is administered at a dose of between 550 mg to 650 mg every three weeks (e.g., 600 mg every three weeks). In some instances, the effective amount of the anti-TIGIT antagonist antibody (e.g., an anti-TIGIT antagonist antibody as disclosed herein, e.g., tiragolumab) is a tiered dose based on a subject's body weight, wherein the subject has a body weight of (a) less than or equal to 15 kg, and the anti-TIGIT antagonist antibody is administered at a dose of 300 mg every three weeks; (b) greater than 15 kg and less than or equal to 40 kg, and the anti-TIGIT antagonist antibody is administered at a dose of 400 mg every three weeks; or (c) greater than 40 kg, and the anti-TIGIT antagonist antibody is administered at a dose of 600 mg every three weeks.

In some instances, the effective amount of the anti-TIGIT antagonist antibody (e.g., an anti-TIGIT antagonist antibody as disclosed herein, e.g., tiragolumab) is a dose of between about 30 mg to about 1200 mg (e.g., between about 30 mg to about 1100 mg, e.g., between about 60 mg to about 1000 mg, e.g., between about 100 mg to about 900 mg, e.g., between about 200 mg to about 800 mg, e.g., between about 300 mg to about 800 mg, e.g., between about 400 mg to about 800 mg, e.g., between about 400 mg to about 750 mg, e.g., between about 450 mg to about 750 mg, e.g., between about 500 mg to about 700 mg, e.g., between about 550 mg to about 650 mg, e.g., 600 mg±10 mg, e.g., 600±6 mg, e.g., 600±5 mg, e.g., 600±3 mg, e.g., 600±1 mg, e.g., 600±0.5 mg, e.g., 600 mg) every three weeks (Q3W) for subject with a body weight greater than 40 kg (e.g., 40.5 kg, 41 kg, 42 kg, 43 kg, 44 kg, 45 kg, 46 kg, 47 kg, 48 kg, 49 kg, 50 kg, 51 kg, 52 kg, 53 kg, 54 kg, 55 kg, 56 kg, 57 kg, 58 kg, 59 kg, 60 kg, 61 kg, 62 kg, 63 kg, 64 kg, 65 kg, 66 kg, 67 kg, 68 kg, 69 kg, 70 kg, 75 kg, 80 kg, 85 kg, 90 kg, 95 kg, 100 kg, 110 kg, 120 kg, 130 kg, 140 kg, 150 kg or more). In some instances, the effective amount of the anti-TIGIT antagonist antibody (e.g., an anti-TIGIT antagonist antibody as disclosed herein, e.g., tiragolumab) is a dose of about 600 mg every three weeks for subject with a body weight greater than 40 kg. In some instances, the effective amount of the anti-TIGIT antagonist antibody (e.g., an anti-TIGIT antagonist antibody as disclosed herein, e.g., tiragolumab) is a dose of between 30 mg to 1200 mg (e.g., between 30 mg to 1100 mg, e.g., between 60 mg to 1000 mg, e.g., between 100 mg to 900 mg, e.g., between 200 mg to 800 mg, e.g., between 300 mg to 800 mg, e.g., between 400 mg to 800 mg, e.g., between 400 mg to 750 mg, e.g., between 450 mg to 750 mg, e.g., between 500 mg to 700 mg, e.g., between 550 mg to 650 mg, e.g., 600 mg±10 mg, e.g., 600±6 mg, e.g., 600±5 mg, e.g., 600±3 mg, e.g., 600±1 mg, e.g., 600±0.5 mg, e.g., 600 mg) every three weeks (Q3W) for subject with a body weight greater than 40 kg (e.g., 40.5 kg, 41 kg, 42 kg, 43 kg, 44 kg, 45 kg, 46 kg, 47 kg, 48 kg, 49 kg, 50 kg, 51 kg, 52 kg, 53 kg, 54 kg, 55 kg, 56 kg, 57 kg, 58 kg, 59 kg, 60 kg, 61 kg, 62 kg, 63 kg, 64 kg, 65 kg, 66 kg, 67 kg, 68 kg, 69 kg, 70 kg, 75 kg, 80 kg, 85 kg, 90 kg, 95 kg, 100 kg, 110 kg, 120 kg, 130 kg, 140 kg, 150 kg or more). In some instances, the effective amount of the anti-TIGIT antagonist antibody (e.g., an anti-TIGIT antagonist antibody as disclosed herein, e.g., tiragolumab) is a dose of 600 mg every three weeks for subject with a body weight greater than 40 kg.

In some instances, the effective amount of the anti-TIGIT antagonist antibody (e.g., an anti-TIGIT antagonist antibody as disclosed herein, e.g., tiragolumab) is a dose of between about 10 mg to about 1000 mg (e.g., between about 20 mg to about 1000 mg, e.g., between about 50 mg to about 900 mg, e.g., between about 100 mg to about 850 mg, e.g., between about 200 mg to about 700 mg, e.g., between about 250 mg to about 600 mg, e.g., between about 300 mg to about 500 mg, e.g., between about 350 mg to about 450 mg, e.g., between about 390 mg to about 410 mg, e.g., about 400 mg) every three weeks (Q3W) for subject with a body weight greater than 15 kg and less than or equal to 40 kg (e.g., 15.1 kg, 15.2 kg, 15.3 kg, 15.4 kg, 15.5 kg, 16 kg, 17 kg, 18 kg, 19 kg, 20 kg, 21 kg, 22 kg, 23 kg, 24 kg, 25 kg, 26 kg, 27 kg, 28 kg, 29 kg, 30 kg, 31 kg, 32 kg, 33 kg, 34 kg, 35 kg, 36 kg, 37 kg, 38 kg, 39 kg, or 39.5 kg). In some instances, the effective amount of the anti-TIGIT antagonist antibody (e.g., an anti-TIGIT antagonist antibody as disclosed herein, e.g., tiragolumab) is a dose of about 400 mg every three weeks (e.g., 400 mg±10 mg, e.g., 400±6 mg, e.g., 400±5 mg, e.g., 400±3 mg, e.g., 400±1 mg, e.g., 400±0.5 mg, e.g., 400 mg every three weeks) for subject with a body weight greater than 15 kg and less than or equal to 40 kg. In some instances, the effective amount of the anti-TIGIT antagonist antibody (e.g., an anti-TIGIT antagonist antibody as disclosed herein, e.g., tiragolumab) is a dose of between 10 mg to 1000 mg (e.g., between 20 mg to 1000 mg, e.g., between 50 mg to 900 mg, e.g., between 100 mg to 850 mg, e.g., between 200 mg to 700 mg, e.g., between 250 mg to 600 mg, e.g., between 300 mg to 500 mg, e.g., between 350 mg to 450 mg, e.g., between 390 mg to 410 mg, e.g., 400 mg) every three weeks (Q3W) for subject with a body weight greater than 15 kg and less than or equal to 40 kg (e.g., 15.1 kg, 15.2 kg, 15.3 kg, 15.4 kg, 15.5 kg, 16 kg, 17 kg, 18 kg, 19 kg, 20 kg, 21 kg, 22 kg, 23 kg, 24 kg, 25 kg, 26 kg, 27 kg, 28 kg, 29 kg, 30 kg, 31 kg, 32 kg, 33 kg, 34 kg, 35 kg, 36 kg, 37 kg, 38 kg, 39 kg, or 39.5 kg). In some instances, the effective amount of the anti-TIGIT antagonist antibody (e.g., an anti-TIGIT antagonist antibody as disclosed herein, e.g., tiragolumab) is a dose of 400 mg every three weeks (e.g., 400 mg±10 mg, e.g., 400±6 mg, e.g., 400±5 mg, e.g., 400±3 mg, e.g., 400±1 mg, e.g., 400±0.5 mg, e.g., 400 mg every three weeks) for subject with a body weight greater than 15 kg and less than or equal to 40 kg.

In some instances, the effective amount of the anti-TIGIT antagonist antibody (e.g., an anti-TIGIT antagonist antibody as disclosed herein, e.g., tiragolumab) is a dose of between about 10 mg to about 1000 mg (e.g., between about 10 mg to about 900 mg, e.g., between about 50 mg to about 900 mg, e.g., between about 100 mg to about 750 mg, e.g., between about 100 mg to about 600 mg, e.g., between about 150 mg to about 500 mg, e.g., between about 200 mg to about 400 mg, e.g., between about 250 mg to about 350 mg, e.g., between about 290 mg to about 310 mg, e.g., about 300 mg) every three weeks (Q3W) for subject with a body weight less than or equal to 15 kg (e.g., 0.5 kg, 1 kg, 1.5 kg, 2.0 kg, 2.5 kg, 3.0 kg, 3.5 kg, 4.0 kg, 4.5 kg, 5.0 kg, 5.5 kg, 6.0 kg, 6.5 kg, 7.0 kg, 7.5 kg, 8.0 kg, 8.5 kg, 9.0 kg, 9.5 kg, 10.0 kg, 10.5 kg, 11.0 kg, 11.5 kg, 12.0 kg, 12.5 kg, 13.0 kg, 13.5 kg, 14.0 kg, 14.5 kg, or 15.0 kg). In some instances, the effective amount of the anti-TIGIT antagonist antibody (e.g., an anti-TIGIT antagonist antibody as disclosed herein, e.g., tiragolumab) is a dose of about 300 mg every three weeks (e.g., 300 mg±10 mg, e.g., 300±6 mg, e.g., 300±5 mg, e.g., 300±3 mg, e.g., 300±1 mg, e.g., 300+0.5 mg, e.g., 300 mg every three weeks) for subject with a body weight less than or equal to 15 kg. In some instances, the effective amount of the anti-TIGIT antagonist antibody (e.g., an anti-TIGIT antagonist antibody as disclosed herein, e.g., tiragolumab) is a dose of between 10 mg to 1000 mg (e.g., between 10 mg to 900 mg, e.g., between 50 mg to 900 mg, e.g., between 100 mg to 750 mg, e.g., between 100 mg to 600 mg, e.g., between 150 mg to 500 mg, e.g., between 200 mg to 400 mg, e.g., between 250 mg to 350 mg, e.g., between 290 mg to 310 mg, e.g., 300 mg) every three weeks (Q3W) for subject with a body weight less than or equal to 15 kg (e.g., 0.5 kg, 1 kg, 1.5 kg, 2.0 kg, 2.5 kg, 3.0 kg, 3.5 kg, 4.0 kg, 4.5 kg, 5.0 kg, 5.5 kg, 6.0 kg, 6.5 kg, 7.0 kg, 7.5 kg, 8.0 kg, 8.5 kg, 9.0 kg, 9.5 kg, 10.0 kg, 10.5 kg, 11.0 kg, 11.5 kg, 12.0 kg, 12.5 kg, 13.0 kg, 13.5 kg, 14.0 kg, 14.5 kg, or 15.0 kg). In some instances, the effective amount of the anti-TIGIT antagonist antibody (e.g., an anti-TIGIT antagonist antibody as disclosed herein, e.g., tiragolumab) is a dose of 300 mg every three weeks (e.g., 300 mg±10 mg, e.g., 300±6 mg, e.g., 300±5 mg, e.g., 300±3 mg, e.g., 300±1 mg, e.g., 300±0.5 mg, e.g., 300 mg every three weeks) for subject with a body weight less than or equal to 15 kg.

In some instances, the effective amount of the anti-TIGIT antagonist antibody (e.g., an anti-TIGIT antagonist antibody as disclosed herein, e.g., tiragolumab) to treat a subject having a cancer is a tiered dose based on a subject's body surface area. In some instances, the effective amount of the anti-TIGIT antagonist antibody (e.g., an anti-TIGIT antagonist antibody as disclosed herein, e.g., tiragolumab) is a tiered dose based on a subject's body surface area, wherein the subject has a body surface area of (a) less than or equal to 0.5 m², and the anti-TIGIT antagonist antibody is administered at a dose of between about 10 mg to about 1000 mg every three weeks (e.g., about 300 mg every three weeks); (b) greater than 0.5 m² and less than or equal to 0.75 m², and the anti-TIGIT antagonist antibody is administered at a dose of between about 10 mg to about 1000 mg every three weeks (e.g., about 350 mg every three weeks); (c) greater than 0.75 m² and less than or equal to 1.25 m², and the anti-TIGIT antagonist antibody is administered at a dose of between about 10 mg to about 1000 mg every three weeks (e.g., about 450 mg every three weeks); or (d) greater than 1.25 m², and the anti-TIGIT antagonist antibody is administered at a dose of between about 30 mg to about 1200 mg every three weeks (e.g., about 600 mg every three weeks). In some instances, the effective amount of the anti-TIGIT antagonist antibody (e.g., an anti-TIGIT antagonist antibody as disclosed herein, e.g., tiragolumab) is a tiered dose based on a subject's body surface area, wherein the subject has a body surface area of (a) less than or equal to 0.5 m², and the anti-TIGIT antagonist antibody is administered at a dose of between about 250 mg to about 350 mg every three weeks (e.g., about 300 mg every three weeks); (b) greater than 0.5 m² and less than or equal to 0.75 m², and the anti-TIGIT antagonist antibody is administered at a dose of between about 300 mg to about 400 mg every three weeks (e.g., about 350 mg every three weeks); or (c) greater than 0.75 m² and less than or equal to 1.25 m², and the anti-TIGIT antagonist antibody is administered at a dose of between about 400 mg to about 500 mg every three weeks (e.g., about 450 mg every three weeks); or (d) greater than 1.25 m², and the anti-TIGIT antagonist antibody is administered at a dose of between about 550 mg to about 650 mg every three weeks (e.g., about 600 mg every three weeks). In some instances, the effective amount of the anti-TIGIT antagonist antibody (e.g., an anti-TIGIT antagonist antibody as disclosed herein, e.g., tiragolumab) is a tiered dose based on a subject's body surface area, wherein the subject has a body surface area of (a) less than or equal to 0.5 m², and the anti-TIGIT antagonist antibody is administered at a dose of about 300 mg every three weeks; (b) greater than 0.5 m² and less than or equal to 0.75 m², and the anti-TIGIT antagonist antibody is administered at a dose of about 400 mg every three weeks; (c) greater than 0.75 m² and less than or equal to 1.25 m², and the anti-TIGIT antagonist antibody is administered at a dose of 450 mg every three weeks; or (d) greater than 1.25 m², and the anti-TIGIT antagonist antibody is administered at a dose of about 600 mg every three weeks. In some instances, the effective amount of the anti-TIGIT antagonist antibody (e.g., an anti-TIGIT antagonist antibody as disclosed herein, e.g., tiragolumab) to treat a subject having a cancer is a tiered dose based on a subject's body surface area. In some instances, the effective amount of the anti-TIGIT antagonist antibody (e.g., an anti-TIGIT antagonist antibody as disclosed herein, e.g., tiragolumab) is a tiered dose based on a subject's body surface area, wherein the subject has a body surface area of (a) less than or equal to 0.5 m², and the anti-TIGIT antagonist antibody is administered at a dose of between 10 mg to 1000 mg every three weeks (e.g., 300 mg every three weeks); (b) greater than 0.5 m² and less than or equal to 0.75 m², and the anti-TIGIT antagonist antibody is administered at a dose of between 10 mg to 1000 mg every three weeks (e.g., 350 mg every three weeks); (c) greater than 0.75 m² and less than or equal to 1.25 m², and the anti-TIGIT antagonist antibody is administered at a dose of between 10 mg to 1000 mg every three weeks (e.g., 450 mg every three weeks); or (d) greater than 1.25 m², and the anti-TIGIT antagonist antibody is administered at a dose of between 30 mg to 1200 mg every three weeks (e.g., 600 mg every three weeks). In some instances, the effective amount of the anti-TIGIT antagonist antibody (e.g., an anti-TIGIT antagonist antibody as disclosed herein, e.g., tiragolumab) is a tiered dose based on a subject's body surface area, wherein the subject has a body surface area of (a) less than or equal to 0.5 m², and the anti-TIGIT antagonist antibody is administered at a dose of between 250 mg to 350 mg every three weeks (e.g., 300 mg every three weeks); (b) greater than 0.5 m² and less than or equal to 0.75 m², and the anti-TIGIT antagonist antibody is administered at a dose of between 300 mg to 400 mg every three weeks (e.g., 350 mg every three weeks); or (c) greater than 0.75 m² and less than or equal to 1.25 m², and the anti-TIGIT antagonist antibody is administered at a dose of between 400 mg to 500 mg every three weeks (e.g., 450 mg every three weeks); or (d) greater than 1.25 m², and the anti-TIGIT antagonist antibody is administered at a dose of between 550 mg to 650 mg every three weeks (e.g., 600 mg every three weeks). In some instances, the effective amount of the anti-TIGIT antagonist antibody (e.g., an anti-TIGIT antagonist antibody as disclosed herein, e.g., tiragolumab) is a tiered dose based on a subject's body surface area, wherein the subject has a body surface area of (a) less than or equal to 0.5 m², and the anti-TIGIT antagonist antibody is administered at a dose of 300 mg every three weeks; (b) greater than 0.5 m² and less than or equal to 0.75 m², and the anti-TIGIT antagonist antibody is administered at a dose of 400 mg every three weeks; (c) greater than 0.75 m² and less than or equal to 1.25 m², and the anti-TIGIT antagonist antibody is administered at a dose of 450 mg every three weeks; or (d) greater than 1.25 m², and the anti-TIGIT antagonist antibody is administered at a dose of 600 mg every three weeks.

In some instances, the effective amount of the anti-TIGIT antagonist antibody (e.g., an anti-TIGIT antagonist antibody as disclosed herein, e.g., tiragolumab) is a dose of between about 30 mg to about 1200 mg (e.g., between about 30 mg to about 1100 mg, e.g., between about 60 mg to about 1000 mg, e.g., between about 100 mg to about 900 mg, e.g., between about 200 mg to about 800 mg, e.g., between about 300 mg to about 800 mg, e.g., between about 400 mg to about 800 mg, e.g., between about 400 mg to about 750 mg, e.g., between about 450 mg to about 750 mg, e.g., between about 500 mg to about 700 mg, e.g., between about 550 mg to about 650 mg, e.g., 600 mg±10 mg, e.g., 600±6 mg, e.g., 600±5 mg, e.g., 600±3 mg, e.g., 600±1 mg, e.g., 600+0.5 mg, e.g., 600 mg) every three weeks (Q3W) for subject with a body surface area greater than 1.25 m² (e.g., 1.25 m², 1.35 m², 1.45 m², 1.50 m², 1.55 m², 1.60 m², 1.65 m², 1.70 m², 1.75 m², 1.80 m², 1.85 m², 1.90 m², 1.95 m², 2.0 m², 2.1 m², 2.2 m², 2.3 m², 2.4 m², 2.5 m², 2.6 m², 2.7 m², 2.8 m², 2.9 m², 3.0 m² or more). In some instances, the effective amount of the anti-TIGIT antagonist antibody (e.g., an anti-TIGIT antagonist antibody as disclosed herein, e.g., tiragolumab) is a dose of about 600 mg every three weeks for subject with a body surface area greater than 1.25 m². In some instances, the effective amount of the anti-TIGIT antagonist antibody (e.g., an anti-TIGIT antagonist antibody as disclosed herein, e.g., tiragolumab) is a dose of between 30 mg to 1200 mg (e.g., between 30 mg to 1100 mg, e.g., between 60 mg to 1000 mg, e.g., between 100 mg to 900 mg, e.g., between 200 mg to 800 mg, e.g., between 300 mg to 800 mg, e.g., between 400 mg to 800 mg, e.g., between 400 mg to 750 mg, e.g., between 450 mg to 750 mg, e.g., between 500 mg to 700 mg, e.g., between 550 mg to 650 mg, e.g., 600 mg±10 mg, e.g., 600±6 mg, e.g., 600±5 mg, e.g., 600±3 mg, e.g., 600+1 mg, e.g., 600±0.5 mg, e.g., 600 mg) every three weeks (Q3W) for subject with a body surface area greater than 1.25 m² (e.g., 1.25 m², 1.35 m², 1.45 m², 1.50 m², 1.55 m², 1.60 m², 1.65 m², 1.70 m², 1.75 m², 1.80 m², 1.85 m², 1.90 m², 1.95 m², 2.0 m², 2.1 m², 2.2 m², 2.3 m², 2.4 m², 2.5 m², 2.6 m², 2.7 m², 2.8 m², 2.9 m², 3.0 m² or more). In some instances, the effective amount of the anti-TIGIT antagonist antibody (e.g., an anti-TIGIT antagonist antibody as disclosed herein, e.g., tiragolumab) is a dose of 600 mg every three weeks for subject with a body surface area greater than 1.25 m².

In some instances, the effective amount of the anti-TIGIT antagonist antibody (e.g., an anti-TIGIT antagonist antibody as disclosed herein, e.g., tiragolumab) is a dose of between about 10 mg to about 1000 mg (e.g., between about 20 mg to about 1000 mg, e.g., between about 50 mg to about 900 mg, e.g., between about 100 mg to about 850 mg, e.g., between about 200 mg to about 700 mg, e.g., between about 250 mg to about 600 mg, e.g., between about 300 mg to about 500 mg, e.g., between about 400 mg to about 500 mg, e.g., between about 440 mg to about 460 mg, e.g., about 450 mg) every three weeks (Q3W) for subject with a body surface area greater than 0.75 m² and less than or equal to 1.25 m² (e.g., 0.76 m², 0.77 m², 0.78 m², 0.79 m², 0.80 m², 0.82 m², 0.84 m², 0.86 m², 0.88 m², 0.90 m², 0.95 m², 1.0 m², 1.05 m², 1.10 m², 1.15 m², 1.20 m², or 1.25 m²). In some instances, the effective amount of the anti-TIGIT antagonist antibody (e.g., an anti-TIGIT antagonist antibody as disclosed herein, e.g., tiragolumab) is a dose of about 450 mg every three weeks (e.g., 450 mg±10 mg, e.g., 450±6 mg, e.g., 450±5 mg, e.g., 450+3 mg, e.g., 450±1 mg, e.g., 450±0.5 mg, e.g., 450 mg every three weeks) for subject with a body surface area greater than 0.75 m² and less than or equal to 1.25 m².

In some instances, the effective amount of the anti-TIGIT antagonist antibody (e.g., an anti-TIGIT antagonist antibody as disclosed herein, e.g., tiragolumab) is a dose of between about 10 mg to about 1000 mg (e.g., between about 20 mg to about 1000 mg, e.g., between about 50 mg to about 900 mg, e.g., between about 100 mg to about 850 mg, e.g., between about 200 mg to about 700 mg, e.g., between about 250 mg to about 600 mg, e.g., between about 300 mg to about 500 mg, e.g., between about 300 mg to about 400 mg, e.g., between about 340 mg to about 360 mg, e.g., about 350 mg) every three weeks (Q3W) for subject with a body surface area greater than 0.5 m² and less than or equal to 0.75 m² (e.g., 0.51 m², 0.52 m², 0.53 m², 0.54 m², 0.55 m², 0.56 m², 0.57 m², 0.58 m², 0.59 m², 0.60 m², 0.61 m², 0.62 m², 0.63 m², 0.64 m², 0.65 m², 0.66 m², 0.67 m², 0.68 m², 0.69 m², 0.70 m², 0.71 m², 0.72 m², 0.73 m², 0.74 m², or 0.75 m²). In some instances, the effective amount of the anti-TIGIT antagonist antibody (e.g., an anti-TIGIT antagonist antibody as disclosed herein, e.g., tiragolumab) is a dose of about 350 mg every three weeks (e.g., 350 mg±10 mg, e.g., 350+6 mg, e.g., 350±5 mg, e.g., 350±3 mg, e.g., 350±1 mg, e.g., 350±0.5 mg, e.g., 350 mg every three weeks) for subject with a body surface area greater than 0.5 m² and less than or equal to 0.75 m².

In some instances, the effective amount of the anti-TIGIT antagonist antibody (e.g., an anti-TIGIT antagonist antibody as disclosed herein, e.g., tiragolumab) is a dose of between about 10 mg to about 1000 mg (e.g., between about 10 mg to about 900 mg, e.g., between about 50 mg to about 900 mg, e.g., between about 100 mg to about 750 mg, e.g., between about 100 mg to about 600 mg, e.g., between about 150 mg to about 500 mg, e.g., between about 200 mg to about 400 mg, e.g., between about 250 mg to about 350 mg, e.g., between about 290 mg to about 310 mg, e.g., about 300 mg) every three weeks (Q3W) for subject with a body surface area less than or equal to 0.5 m² (e.g., 0.02 m², 0.04 m², 0.06 m², 0.08 m², 0.1 m², 0.15 m², 0.20 m², 0.25 m², 0.30 m², 0.35 m², 0.40 m², 0.45 m², or 0.50 m²). In some instances, the effective amount of the anti-TIGIT antagonist antibody (e.g., an anti-TIGIT antagonist antibody as disclosed herein, e.g., tiragolumab) is a dose of about 300 mg every three weeks (e.g., 300 mg±10 mg, e.g., 300+6 mg, e.g., 300±5 mg, e.g., 300±3 mg, e.g., 300±1 mg, e.g., 300±0.5 mg, e.g., 300 mg every three weeks) for subject with a body surface area less than or equal to 0.5 m².

In some instances, the effective amount of the anti-TIGIT antagonist antibody (e.g., an anti-TIGIT antagonist antibody as disclosed herein, e.g., tiragolumab) is a dose (e.g., a fixed dose) of between about 10 mg to about 1000 mg (e.g., between about 20 mg to about 1000 mg, e.g., between about 50 mg to about 900 mg, e.g., between about 100 mg to about 850 mg, e.g., between about 200 mg to about 800 mg, e.g., between about 300 mg to about 600 mg, e.g., between about 400 mg to about 500 mg, e.g., between about 405 mg to about 450 mg, e.g., between about 410 mg to about 430 mg, e.g., about 420 mg) every two weeks (Q2W). In some instances, the effective amount of the anti-TIGIT antagonist antibody (e.g., an anti-TIGIT antagonist antibody as disclosed herein, e.g., tiragolumab) is a dose of about 420 mg every two weeks (e.g., 420 mg±10 mg, e.g., 420±6 mg, e.g., 420±5 mg, e.g., 420±3 mg, e.g., 420±1 mg, e.g., 420±0.5 mg, e.g., 420 mg every two weeks). In some instances, the method comprises administering to the subject or population of subjects the anti-TIGIT antagonist antibody at a dose of about 300 mg to about 600 mg every two weeks. In some instances, the method comprises administering to the subject or population of subjects the anti-TIGIT antagonist antibody at a dose of 300 mg to 600 mg every two weeks. In some instances the method comprises administering to the subject or population of subjects the anti-TIGIT antagonist antibody at a dose of about 420 every two weeks. In some instances the method comprises administering to the subject or population of subjects the anti-TIGIT antagonist antibody at a dose of 420 every two weeks. In some instances, the dose of the anti-TIGIT antagonist antibody is a fixed dose.

In some instances, the effective amount of the anti-TIGIT antagonist antibody (e.g., an anti-TIGIT antagonist antibody as disclosed herein, e.g., tiragolumab) is a dose of between about 30 mg to about 1200 mg (e.g., between about 30 mg to about 1100 mg, e.g., between about 60 mg to about 1000 mg, e.g., between about 100 mg to about 900 mg, e.g., between about 200 mg to about 800 mg, e.g., between about 300 mg to about 800 mg, e.g., between about 400 mg to about 800 mg, e.g., between about 400 mg to about 750 mg, e.g., between about 450 mg to about 750 mg, e.g., between about 500 mg to about 700 mg, e.g., between about 550 mg to about 650 mg, e.g., 600 mg±10 mg, e.g., 600±6 mg, e.g., 600±5 mg, e.g., 600±3 mg, e.g., 600±1 mg, e.g., 600±0.5 mg, e.g., 600 mg) every three weeks (Q3W). In some instances, the effective amount of the anti-TIGIT antagonist antibody (e.g., an anti-TIGIT antagonist antibody as disclosed herein, e.g., tiragolumab) is a dose of about 600 mg every three weeks. In some instances, the effective amount of the anti-TIGIT antagonist antibody (e.g., an anti-TIGIT antagonist antibody as disclosed herein, e.g., tiragolumab) is a dose of between about 200 mg to about 2000 mg (e.g., between about 200 mg to about 2000 mg, e.g., between about 400 mg to about 1900 mg, e.g., between about 500 mg to about 1800 mg, e.g., between about 600 mg to about 1700 mg, e.g., between about 700 mg to about 1400 mg, e.g., between about 800 mg to about 1600 mg, e.g., between about 900 mg to about 1500 mg, e.g., between about 1000 mg to about 1400 mg, e.g., between about 1050 mg to about 1350 mg, e.g., between about 1100 mg to about 1300 mg, e.g., between about 1150 mg to about 1250 mg, e.g., between about 1175 mg to about 1225 mg, e.g., between about 1190 mg to about 1210 mg, e.g., about 1200 mg, e.g., 1200 mg±10 mg, e.g., 1200 6 mg, e.g., 1200±5 mg, e.g., 1200±3 mg, e.g., 1200±1 mg, e.g., 1200±0.5 mg, e.g., 1200 mg) every three weeks (Q3W). In some instances, the effective amount of the anti-TIGIT antagonist antibody (e.g., an anti-TIGIT antagonist antibody as disclosed herein, e.g., tiragolumab) is a dose of about 1200 mg every three weeks. In some instances, the effective amount of the anti-TIGIT antagonist antibody (e.g., an anti-TIGIT antagonist antibody as disclosed herein, e.g., tiragolumab) is a dose of 1200 mg every three weeks.

In some instances, the effective amount of the anti-TIGIT antagonist antibody (e.g., an anti-TIGIT antagonist antibody as disclosed herein, e.g., tiragolumab) is a dose of between about 200 mg to about 2000 mg (e.g., between about 200-300 mg, between about 300-400 mg, between about 400-500 mg, between about 500-600 mg, between about 600-700 mg, between about 700-800 mg, between about 800-900 mg, between about 900-1000 mg, between about 1000-1100 mg, between about 1100-1200 mg, between about 1200-1300 mg, between about 1300-1400 mg, between about 1400-1500 mg, between about 1500-1600 mg, between about 1600-1700 mg, between about 1700-1800 mg, between about 1800-1900 mg, or between about 1900-2000 mg, e.g., between about 200 mg to about 1600 mg, e.g., between about 250 mg to about 1600 mg, e.g., between about 300 mg to about 1600 mg, e.g., between about 400 mg to about 1500 mg, e.g., between about 500 mg to about 1400 mg, e.g., between about 600 mg to about 1200 mg, e.g., between about 700 mg to about 1100 mg, e.g., between about 800 mg to about 1000 mg, e.g., between about 800 mg to about 900 mg, e.g., about 200, about 250, about 300, about 350, about 400, about 450, about 500, about 550, about 600, about 650, about 700, about 750, about 800, about 850, about 900, about 950, about 1000, about 1050, about 1100, about 1150, about 1200, about 1250, about 1300, about 1350, about 1400, about 1450, about 1500, about 1550, about 1600 mg, about 1650 mg, about 1700 mg, about 1750 mg, about 1800 mg, about 1850 mg, about 1900 mg, about 1950 mg, or about 2000 mg, e.g., about 800, about 810, about 820, about 830, about 840, about 850, about 860, about 870, about 880, about 890, or about 900 mg) every four weeks (Q4W). In some instances, the effective amount of the anti-TIGIT antagonist antibody (e.g., an anti-TIGIT antagonist antibody as disclosed herein, e.g., tiragolumab) is about 700 mg to about 1000 mg every four weeks. In some instances, the effective amount of the anti-TIGIT antagonist antibody (e.g., an anti-TIGIT antagonist antibody as disclosed herein, e.g., tiragolumab) is 700 mg to 1000 mg every four weeks. In some instances, the effective amount of the anti-TIGIT antagonist antibody (e.g., an anti-TIGIT antagonist antibody as disclosed herein, e.g., tiragolumab) is about 840 mg every four weeks. In some instances, the effective amount of the anti-TIGIT antagonist antibody (e.g., an anti-TIGIT antagonist antibody as disclosed herein, e.g., tiragolumab) is 840 mg every four weeks. The 840 mg Q4W dosing regimen is supported by results from PK modeling and simulation and exposure-safety analyses. Briefly, the average concentration following the 840 mg Q4W dosing regimen is similar to that of the 600 mg every 3 weeks dosing regimen, which was evaluated in previous studies. The Cmax of the 840 mg Q4W dosing regimen was simulated to be 28% higher at steady state, relative to the Cmax for the 600 mg every 3 weeks dosing regimen, but falls within the range of observed exposure of the highest administered dose in the clinic (1200 mg every 3 weeks). A preliminary analysis of the tiragolumab exposure-safety relationship based on previous observations (tiragolumab doses of 2-1200 mg every 3 weeks administered as monotherapy or in combination with atezolizumab 1200 mg every 3 weeks) suggest that tiragolumab exhibits a flat exposure-safety relationship. In summary, the 840 mg Q4W dosing regimen can provide comparable safety and efficacy as the 600 mg every-3-weeks dosing regimen, given that the predicted exposure is within the range of observed efficacious exposures and tiragolumab exhibits a flat exposure-safety relationship.

In some instances, the effective amount of anti-TIGIT antagonist antibody (e.g., an anti-TIGIT antagonist antibody as disclosed herein, e.g., tiragolumab) is a dose of about 840 mg every four weeks (e.g., 840 mg±10 mg, e.g., 840±6 mg, e.g., 840±5 mg, e.g., 840±3 mg, e.g., 840±1 mg, e.g., 840+0.5 mg, e.g., 840 mg every four weeks). In some instances, the effective amount of the anti-TIGIT antagonist antibody (e.g., an anti-TIGIT antagonist antibody as disclosed herein, e.g., tiragolumab) is a dose of between about 200 mg to about 2000 mg (e.g., between about 200 mg to about 2000 mg, e.g., between about 400 mg to about 1900 mg, e.g., between about 500 mg to about 1800 mg, e.g., between about 600 mg to about 1700 mg, e.g., between about 700 mg to about 1400 mg, e.g., between about 800 mg to about 1600 mg, e.g., between about 900 mg to about 1500 mg, e.g., between about 1000 mg to about 1400 mg, e.g., between about 1050 mg to about 1350 mg, e.g., between about 1100 mg to about 1300 mg, e.g., between about 1150 mg to about 1250 mg, e.g., between about 1175 mg to about 1225 mg, e.g., between about 1190 mg to about 1210 mg (e.g., between 200 mg to 2000 mg, e.g., between 400 mg to 1900 mg, e.g., between 500 mg to 1800 mg, e.g., between 600 mg to 1700 mg, e.g., between 700 mg to 1400 mg, e.g., between 800 mg to 1600 mg, e.g., between 900 mg to 1500 mg, e.g., between 1000 mg to 1400 mg, e.g., between 1050 mg to 1350 mg, e.g., between 1100 mg to 1300 mg, e.g., between 1150 mg to 1250 mg, e.g., between 1175 mg to 1225 mg, e.g., between 1190 mg to 1210 mg), e.g., about 1200 mg, e.g., 1200 mg±10 mg, e.g., 1200±6 mg, e.g., 1200±5 mg, e.g., 1200±3 mg, e.g., 1200±1 mg, e.g., 1200±0.5 mg, e.g., 1200 mg) every four weeks (Q4W). In some instances, the effective amount of the anti-TIGIT antagonist antibody (e.g., an anti-TIGIT antagonist antibody as disclosed herein, e.g., tiragolumab) is a dose of about 1200 mg every four weeks.

In some instances, the dose of the anti-TIGIT antagonist antibody (e.g., an anti-TIGIT antagonist antibody as disclosed herein, e.g., tiragolumab) administered in a combination therapy (e.g., a combination treatment with a PD-1 axis binding antagonist (e.g., anti-PD-L1 antagonist antibody (e.g., atezolizumab) or anti-PD-1 antagonist antibody (e.g., pembrolizumab)) may be reduced as compared to a standard dose of the anti-TIGIT antagonist antibody administered as a monotherapy.

In some instances, the anti-TIGIT antagonist antibody (e.g., an anti-TIGIT antagonist antibody as disclosed herein, e.g., tiragolumab) is administered intravenously. Alternatively, in some embodiments, the anti-TIGIT antagonist antibody (e.g., an anti-TIGIT antagonist antibody as disclosed herein, e.g., tiragolumab) is administered subcutaneously. In some instances, tiragolumab is administered to the patient intravenously at a dose of about 420 mg every 2 weeks, about 1200 mg every 3 weeks, or about 1200 mg of every 4 weeks. In some instances, tiragolumab is administered to the patient intravenously at a dose of 420 mg every 2 weeks, 1200 mg every 3 weeks, or 1200 mg of every 4 weeks.

In some instances, a subject is administered a total of 1 to 20 doses of an anti-TIGIT antagonist antibody (e.g., an anti-TIGIT antagonist antibody as disclosed herein, e.g., tiragolumab), e.g., 1, 2, 3, 4, 5, 6, 7, 8, 9, 10, 11, 12, 13, 14, 15, 16, 17, 18, 19, or 20 doses. In some instances, a subject is administered a total of 1 to 50 doses of an anti-TIGIT antagonist antibody (e.g., an anti-TIGIT antagonist antibody as disclosed herein, e.g., tiragolumab), e.g., 1 to 50 doses, 1 to 45 doses, 1 to 40 doses, 1 to 35 doses, 1 to 30 doses, 1 to 25 doses, 1 to 20 doses, 1 to 15 doses, 1 to 10 doses, 1 to 5 doses, 2 to 50 doses, 2 to 45 doses, 2 to 40 doses, 2 to 35 doses, 2 to 30 doses, 2 to 25 doses, 2 to 20 doses, 2 to 15 doses, 2 to 10 doses, 2 to 5 doses, 3 to 50 doses, 3 to 45 doses, 3 to 40 doses, 3 to 35 doses, 3 to 30 doses, 3 to 25 doses, 3 to 20 doses, 3 to 15 doses, 3 to 10 doses, 3 to 5 doses, 4 to 50 doses, 4 to 45 doses, 4 to 40 doses, 4 to 35 doses, 4 to 30 doses, 4 to 25 doses, 4 to 20 doses, 4 to 15 doses, 4 to 10 doses, 4 to 5 doses, 5 to 50 doses, 5 to 45 doses, 5 to 40 doses, 5 to 35 doses, 5 to 30 doses, 5 to 25 doses, 5 to 20 doses, 5 to 15 doses, 5 to 10 doses, 10 to 50 doses, 10 to 45 doses, 10 to 40 doses, 10 to 35 doses, 10 to 30 doses, 10 to 25 doses, 10 to 20 doses, 10 to 15 doses, 15 to 50 doses, 15 to 45 doses, 15 to 40 doses, 15 to 35 doses, 15 to 30 doses, 15 to 25 doses, 15 to 20 doses, 20 to 50 doses, 20 to 45 doses, 20 to 40 doses, 20 to 35 doses, 20 to 30 doses, 20 to 25 doses, 25 to 50 doses, 25 to 45 doses, 25 to 40 doses, 25 to 35 doses, 25 to 30 doses, 30 to 50 doses, 30 to 45 doses, 30 to 40 doses, 30 to 35 doses, 35 to 50 doses, 35 to 45 doses, 35 to 40 doses, 40 to 50 doses, 40 to 45 doses, or 45 to 50 doses. In particular instances, the doses may be administered intravenously.

ii. Dosing of PD-1 Axis Binding Antagonists

As a general proposition, the therapeutically effective amount of a PD-1 axis binding antagonist (e.g., atezolizumab) administered to a human will be in the range of about 0.01 to about 50 mg/kg of patient body weight, whether by one or more administrations.

In some exemplary embodiments, the PD-1 axis binding antagonist is administered in a dose of about 0.01 to about 45 mg/kg, about 0.01 to about 40 mg/kg, about 0.01 to about 35 mg/kg, about 0.01 to about 30 mg/kg, about 0.01 to about 25 mg/kg, about 0.01 to about 20 mg/kg, about 0.01 to about 15 mg/kg, about 0.01 to about 10 mg/kg, about 0.01 to about 5 mg/kg, or about 0.01 to about 1 mg/kg administered daily, weekly, every two weeks, every three weeks, or every four weeks, for example. In some exemplary embodiments, the PD-1 axis binding antagonist is administered in a dose of 0.01 to 45 mg/kg, 0.01 to 40 mg/kg, 0.01 to 35 mg/kg, 0.01 to 30 mg/kg, 0.01 to 25 mg/kg, 0.01 to 20 mg/kg, 0.01 to 15 mg/kg, 0.01 to 10 mg/kg, 0.01 to 5 mg/kg, or 0.01 to 1 mg/kg administered daily, weekly, every two weeks, every three weeks, or every four weeks, for example.

In some instances, the PD-1 axis binding antagonist is administered on about Day 1 (e.g., Day −3, Day −2, Day −1, Day 1, Day 2, or Day 3) of a dosing cycle.

In some instances, the effective amount of the PD-1 axis binding antagonist (e.g., anti-PD-L1 antagonist antibody (e.g., atezolizumab) or anti-PD-1 antagonist antibody (e.g., pembrolizumab)) is a dose (e.g., a fixed dose) of between about 20 mg to about 1600 mg (e.g., between about 40 mg to about 1500 mg, e.g., between about 200 mg to about 1400 mg, e.g., between about 300 mg to about 1400 mg, e.g., between about 400 mg to about 1400 mg, e.g., between about 500 mg to about 1300 mg, e.g., between about 600 mg to about 1200 mg, e.g., between about 700 mg to about 1100 mg, e.g., between about 800 mg to about 1000 mg, e.g., between about 800 mg to about 900 mg, e.g., about 800, about 810, about 820, about 830, about 840, about 850, about 860, about 870, about 880, about 890, or about 900 mg) every two weeks (Q2W). In some instances, the effective amount of the PD-1 axis binding antagonist (e.g., anti-PD-L1 antagonist antibody (e.g., atezolizumab) or anti-PD-1 antagonist antibody (e.g., pembrolizumab)) is a dose (e.g., a fixed dose) of between 20 mg to 1600 mg (e.g., between 40 mg to 1500 mg, e.g., between 200 mg to 1400 mg, e.g., between 300 mg to 1400 mg, e.g., between 400 mg to 1400 mg, e.g., between 500 mg to 1300 mg, e.g., between 600 mg to 1200 mg, e.g., between 700 mg to 1100 mg, e.g., between 800 mg to 1000 mg, e.g., between 800 mg to 900 mg, e.g., 800, 810, 820, 830, 840, 850, 860, 870, 880, 890, or 900 mg) every two weeks (Q2W). In some instances, the effective amount of the PD-1 axis binding antagonist (e.g., anti-PD-L1 antagonist antibody (e.g., atezolizumab) or anti-PD-1 antagonist antibody (e.g., pembrolizumab)) is a dose of about 840 mg every two weeks (e.g., 840 mg±10 mg, e.g., 840±6 mg, e.g., 840±5 mg, e.g., 840±3 mg, e.g., 840±1 mg, e.g., 840±0.5 mg, e.g., 840 mg every two weeks). In some instances, the effective amount of atezolizumab is a dose of about 840 mg every two weeks.

In some instances, the effective amount of the PD-1 axis binding antagonist (e.g., anti-PD-1 antagonist antibody (e.g., pembrolizumab) or anti-PD-L1 antagonist antibody (e.g., atezolizumab)) is a dose of between about 0.01 mg/kg to about 50 mg/kg of the subject's body weight (e.g., between about 0.01 mg/kg to about 45 mg/kg, e.g., between about 0.1 mg/kg to about 40 mg/kg, e.g., between about 1 mg/kg to about 35 mg/kg, e.g., between about 2.5 mg/kg to about 30 mg/kg, e.g., between about 5 mg/kg to about 25 mg/kg, e.g., between about 5 mg/kg to about 15 mg/kg, e.g., between about 7.5 mg/kg to about 12.5 mg/kg, e.g., about 10±2 mg/kg, about 10±1 mg/kg, about 10±0.5 mg/kg, about 10±0.2 mg/kg, or about 10±0.1 mg/kg, e.g., about 10 mg/kg) every two weeks. In some instances, the effective amount of the PD-1 axis binding antagonist (e.g., anti-PD-1 antagonist antibody (e.g., pembrolizumab) or anti-PD-L1 antagonist antibody (e.g., atezolizumab)) is a dose of between about 0.01 mg/kg to about 10 mg/kg of the subject's body weight (e.g., between about 0.1 mg/kg to about 10 mg/kg, e.g., between about 0.5 mg/kg to about 10 mg/kg, e.g., between about 1 mg/kg to about 10 mg/kg, e.g., between about 2.5 mg/kg to about 10 mg/kg, e.g., between about 5 mg/kg to about 10 mg/kg, e.g., between about 7.5 mg/kg to about 10 mg/kg, e.g., between about 8 mg/kg to about 10 mg/kg, e.g., between about 9 mg/kg to about 10 mg/kg, e.g., between about 9.5 mg/kg to about 10 mg/kg, e.g., about 10±1 mg/kg, e.g., about 10±0.5 mg/kg, e.g., about 10±0.2 mg/kg, e.g., about 10±0.1 mg/kg, e.g., about 10 mg/kg) every two weeks. In some instances, the effective amount of the PD-1 axis binding antagonist (e.g., anti-PD-1 antagonist antibody (e.g., pembrolizumab) or anti-PD-L1 antagonist antibody (e.g., atezolizumab)) is a dose of between 0.01 mg/kg to 50 mg/kg of the subject's body weight (e.g., between 0.01 mg/kg to 45 mg/kg, e.g., between 0.1 mg/kg to 40 mg/kg, e.g., between 1 mg/kg to 35 mg/kg, e.g., between 2.5 mg/kg to 30 mg/kg, e.g., between 5 mg/kg to 25 mg/kg, e.g., between 5 mg/kg to 15 mg/kg, e.g., between 7.5 mg/kg to 12.5 mg/kg, e.g., 10±2 mg/kg, 10±1 mg/kg, 10±0.5 mg/kg, 10 0.2 mg/kg, or 10±0.1 mg/kg, e.g., 10 mg/kg) every two weeks. In some instances, the effective amount of the PD-1 axis binding antagonist (e.g., anti-PD-1 antagonist antibody (e.g., pembrolizumab) or anti-PD-L1 antagonist antibody (e.g., atezolizumab)) is a dose of between 0.01 mg/kg to 10 mg/kg of the subject's body weight (e.g., between 0.1 mg/kg to 10 mg/kg, e.g., between 0.5 mg/kg to 10 mg/kg, e.g., between 1 mg/kg to 10 mg/kg, e.g., between 2.5 mg/kg to 10 mg/kg, e.g., between 5 mg/kg to 10 mg/kg, e.g., between 7.5 mg/kg to 10 mg/kg, e.g., between 8 mg/kg to 10 mg/kg, e.g., between 9 mg/kg to 10 mg/kg, e.g., between 9.5 mg/kg to 10 mg/kg, e.g., 10±1 mg/kg, e.g., 10±0.5 mg/kg, e.g., 10±0.2 mg/kg, e.g., 10±0.1 mg/kg, e.g., 10 mg/kg) every two weeks. In some instances, the effective amount of pembrolizumab is a dose of about 10 mg/kg every two weeks. In some instances, the effective amount of pembrolizumab is a dose of 10 mg/kg every two weeks.

In some instances, the effective amount of the PD-1 axis binding antagonist (e.g., anti-PD-L1 antagonist antibody (e.g., atezolizumab)) to treat a subject having a cancer is a dose of between about 0.01 mg/kg to about 50 mg/kg of the subject's body weight (e.g., between about 0.01 mg/kg to about 45 mg/kg, e.g., between about 0.1 mg/kg to about 40 mg/kg, e.g., between about 1 mg/kg to about 35 mg/kg, e.g., between about 2.5 mg/kg to about 30 mg/kg, e.g., between about 5 mg/kg to about 25 mg/kg, e.g., between about 10 mg/kg to about 20 mg/kg, e.g., between about 12.5 mg/kg to about 15 mg/kg, e.g., about 15±2 mg/kg, about 15±1 mg/kg, about 15±0.5 mg/kg, about 15±0.2 mg/kg, or about 15±0.1 mg/kg, e.g., about 15 mg/kg) every three weeks. In some instances, the effective amount of the PD-1 axis binding antagonist (e.g., anti-PD-L1 antagonist antibody (e.g., atezolizumab)) is a dose of between about 0.01 mg/kg to about 15 mg/kg of the subject's body weight (e.g., between about 0.1 mg/kg to about 15 mg/kg, e.g., between about 0.5 mg/kg to about 15 mg/kg, e.g., between about 1 mg/kg to about 15 mg/kg, e.g., between about 2.5 mg/kg to about 15 mg/kg, e.g., between about 5 mg/kg to about 15 mg/kg, e.g., between about 7.5 mg/kg to about 15 mg/kg, e.g., between about 10 mg/kg to about 15 mg/kg, e.g., between about 12.5 mg/kg to about 15 mg/kg, e.g., between about 14 mg/kg to about 15 mg/kg, e.g., about 15±1 mg/kg, e.g., about 15±0.5 mg/kg, e.g., about 15±0.2 mg/kg, e.g., about 15±0.1 mg/kg, e.g., about 15 mg/kg) every three weeks. In some instances, the effective amount of the PD-1 axis binding antagonist (e.g., anti-PD-L1 antagonist antibody (e.g., atezolizumab)) to treat a subject having a cancer is a dose of between 0.01 mg/kg to 50 mg/kg of the subject's body weight (e.g., between 0.01 mg/kg to 45 mg/kg, e.g., between 0.1 mg/kg to 40 mg/kg, e.g., between 1 mg/kg to 35 mg/kg, e.g., between 2.5 mg/kg to 30 mg/kg, e.g., between 5 mg/kg to 25 mg/kg, e.g., between 10 mg/kg to 20 mg/kg, e.g., between 12.5 mg/kg to 15 mg/kg, e.g., 15±2 mg/kg, 15±1 mg/kg, 15±0.5 mg/kg, 15±0.2 mg/kg, or 15±0.1 mg/kg, e.g., 15 mg/kg) every three weeks. In some instances, the effective amount of the PD-1 axis binding antagonist (e.g., anti-PD-L1 antagonist antibody (e.g., atezolizumab)) is a dose of between 0.01 mg/kg to 15 mg/kg of the subject's body weight (e.g., between 0.1 mg/kg to 15 mg/kg, e.g., between 0.5 mg/kg to 15 mg/kg, e.g., between 1 mg/kg to 15 mg/kg, e.g., between 2.5 mg/kg to 15 mg/kg, e.g., between 5 mg/kg to 15 mg/kg, e.g., between 7.5 mg/kg to 15 mg/kg, e.g., between 10 mg/kg to 15 mg/kg, e.g., between 12.5 mg/kg to 15 mg/kg, e.g., between 14 mg/kg to 15 mg/kg, e.g., 15±1 mg/kg, e.g., 15 0.5 mg/kg, e.g., 15±0.2 mg/kg, e.g., 15±0.1 mg/kg, e.g., 15 mg/kg) every three weeks. In some instances, the effective amount of PD-1 axis binding antagonist (e.g., anti-PD-L1 antagonist antibody (e.g., atezolizumab)) is a dose of about 15 mg/kg administered every three weeks. In some instances, the effective amount of PD-1 axis binding antagonist (e.g., anti-PD-L1 antagonist antibody (e.g., atezolizumab)) is a dose of about 15 mg/kg administered every three weeks with a maximum dose of 1200 mg every three weeks. In some instances, the dose of the PD-1 axis binding antagonist (e.g., anti-PD-L1 antagonist antibody (e.g., atezolizumab)) administered in a combination therapy (e.g., a combination treatment with an anti-TIGIT antagonist antibody, such as an anti-TIGIT antagonist antibody disclosed herein, e.g., tiragolumab) may be reduced as compared to a standard dose of the PD-1 axis binding antagonist administered as a monotherapy. In some embodiments, the PD-1 axis binding antagonist (e.g., anti-PD-L1 antagonist antibody (e.g., atezolizumab)) is administered at a maximum dose of 1200 mg every three weeks.

In some instances, the effective amount of the PD-1 axis binding antagonist (e.g., anti-PD-L1 antagonist antibody (e.g., atezolizumab) or anti-PD-1 antagonist antibody (e.g., pembrolizumab)) is a dose of between about 80 mg to about 2000 mg (e.g., between about 100 mg to about 1600 mg, e.g., between about 200 mg to about 1600 mg, e.g., between about 300 mg to about 1600 mg, e.g., between about 400 mg to about 1600 mg, e.g., between about 500 mg to about 1600 mg, e.g., between about 600 mg to about 1600 mg, e.g., between about 700 mg to about 1600 mg, e.g., between about 800 mg to about 1600 mg, e.g., between about 900 mg to about 1500 mg, e.g., between about 1000 mg to about 1400 mg, e.g., between about 1050 mg to about 1350 mg, e.g., between about 1100 mg to about 1300 mg, e.g., between about 1150 mg to about 1250 mg, e.g., between about 1175 mg to about 1225 mg, e.g., between about 1190 mg to about 1210 mg, e.g., 1200 mg±5 mg, e.g., 1200±2.5 mg, e.g., 1200+1.0 mg, e.g., 1200±0.5 mg, e.g., 1200 mg) every three weeks (Q3W). In some instances, the effective amount of the PD-1 axis binding antagonist (e.g., anti-PD-L1 antagonist antibody (e.g., atezolizumab) or anti-PD-1 antagonist antibody (e.g., pembrolizumab)) is a dose of about 1200 mg every three weeks (e.g., 1200 mg±10 mg, e.g., 1200±6 mg, e.g., 1200±5 mg, e.g., 1200±3 mg, e.g., 1200±1 mg, e.g., 1200±0.5 mg, e.g., 1200 mg every three weeks). In some instances, the effective amount of atezolizumab is a dose of 1200 mg every three weeks.

In some instances, the effective amount of the PD-1 axis binding antagonist (e.g., anti-PD-L1 antagonist antibody (e.g., atezolizumab) or anti-PD-1 antagonist antibody (e.g., pembrolizumab)) is a dose of between about 10 mg and about 800 mg (e.g., between about 10 mg and about 800 mg, e.g., between about 20 mg and about 700 mg, e.g., between about 50 mg and about 600 mg, e.g., between about 75 mg and about 500 mg, e.g., between about 100 mg and about 400 mg, e.g., between about 100 mg and about 300 mg, e.g., between about 125 mg and about 275 mg, e.g., between about 150 mg and about 250 mg, e.g., between about 175 mg and about 225 mg, e.g., between about 190 mg and about 210 mg, e.g., about 200 mg±10 mg, e.g., 200 mg±7.5 mg, e.g., 200 mg±5 mg, e.g., 200±2.5 mg, e.g., 200±1.0 mg, e.g., 200±0.5 mg, e.g., 200 mg) every three weeks (Q3W). In some instances, the effective amount of the PD-1 axis binding antagonist (e.g., anti-PD-L1 antagonist antibody (e.g., atezolizumab) or anti-PD-1 antagonist antibody (e.g., pembrolizumab)) is a dose of about 200 mg every three weeks (e.g., 200 mg±10 mg, e.g., 200±6 mg, e.g., 200±5 mg, e.g., 200±3 mg, e.g., 200±1 mg, e.g., 200±0.5 mg, e.g., 200 mg every three weeks). In some instances, the effective amount of the anti-PD-1 antagonist antibody (e.g., pembrolizumab)) is a dose of about 200 mg every three weeks (e.g., 200 mg±10 mg, e.g., 200±6 mg, e.g., 200±5 mg, e.g., 200±3 mg, e.g., 200±1 mg, e.g., 200±0.5 mg, e.g., 200 mg every three weeks). In some instances, the effective amount of the PD-1 axis binding antagonist (e.g., anti-PD-L1 antagonist antibody (e.g., atezolizumab) or anti-PD-1 antagonist antibody (e.g., pembrolizumab)) is a dose of between 10 mg and 800 mg (e.g., between 10 mg and 800 mg, e.g., between 20 mg and 700 mg, e.g., between 50 mg and 600 mg, e.g., between 75 mg and 500 mg, e.g., between 100 mg and 400 mg, e.g., between 100 mg and 300 mg, e.g., between 125 mg and 275 mg, e.g., between 150 mg and 250 mg, e.g., between 175 mg and 225 mg, e.g., between 190 mg and 210 mg, e.g., 200 mg±10 mg, e.g., 200 mg±7.5 mg, e.g., 200 mg±5 mg, e.g., 200±2.5 mg, e.g., 200±1.0 mg, e.g., 200±0.5 mg, e.g., 200 mg) every three weeks (Q3W). In some instances, the effective amount of the PD-1 axis binding antagonist (e.g., anti-PD-L1 antagonist antibody (e.g., atezolizumab) or anti-PD-1 antagonist antibody (e.g., pembrolizumab)) is a dose of 200 mg every three weeks (e.g., 200 mg±10 mg, e.g., 200±6 mg, e.g., 200±5 mg, e.g., 200±3 mg, e.g., 200±1 mg, e.g., 200±0.5 mg, e.g., 200 mg every three weeks). In some instances, the effective amount of the anti-PD-1 antagonist antibody (e.g., pembrolizumab)) is a dose of 200 mg every three weeks (e.g., 200 mg±10 mg, e.g., 200±6 mg, e.g., 200±5 mg, e.g., 200±3 mg, e.g., 200±1 mg, e.g., 200±0.5 mg, e.g., 200 mg every three weeks). In some instances, the effective amount of pembrolizumab is a dose of 200 mg every three weeks. In some instances, the effective amount of pembrolizumab is a dose of 200 mg every three weeks.

In some instances, the effective amount of the PD-1 axis binding antagonist (e.g., anti-PD-L1 antagonist antibody (e.g., atezolizumab) or anti-PD-1 antagonist antibody (e.g., pembrolizumab)) is a dose of between about 80 mg to about 3000 mg (e.g., between about 80-200 mg, between about 200-400 mg, between about 400-600 mg, between about 600-800 mg, between about 800-1000 mg, between about 1000-1200 mg, between about 1200-1400 mg, between about 1400-1600 mg, between about 1600-1800 mg, between about 1800-2000 mg, between about 2200-2400 mg, between about 2400-2600 mg, between about 2600-2800 mg, or between about 2800-3000 mg, e.g., between about 100 mg and about 3000 mg, e.g., between about 200 mg and about 2900 mg, e.g., between about 500 mg to about 2800 mg, e.g., between about 600 mg to about 2700 mg, e.g., between about 650 mg to about 2600 mg, e.g., between about 700 mg to about 2500 mg, e.g., between about 1000 mg to about 2400 mg, e.g., between about 1100 mg to about 2300 mg, e.g., between about 1200 mg to about 2200 mg, e.g., between about 1300 mg to about 2100 mg, e.g., between about 1400 mg to about 2000 mg, e.g., between about 1500 mg to about 1900 mg, e.g., between about 1600 mg to about 1800 mg, e.g., between about 1620 mg to about 1700 mg, e.g., between about 1640 mg to about 1690 mg, e.g., between about 1660 mg to about 1680 mg, about 1680 mg, e.g., about 80 mg, about 200 mg, about 400 mg, about 600 mg, about 800 mg, about 1000 mg, about 1200 mg, about 1400 mg, about 1600 mg, about 1800 mg, about 2000 mg, about 2200 mg, about 2400 mg, about 2600 mg, about 2800 mg, or about 3000 mg, e.g., about 1600 mg, about 1610 mg, about 1620 mg, about 1630 mg, about 1640 mg, about 1650 mg, about 1660 mg, about 1670 mg, about 1680 mg, about 1690 mg, or about 1700 mg) every four weeks (Q4W). In some instances, the effective amount of the PD-1 axis binding antagonist (e.g., anti-PD-L1 antagonist antibody (e.g., atezolizumab) or anti-PD-1 antagonist antibody (e.g., pembrolizumab)) is a dose of between 500 mg to 3000 mg (e.g., between 500 mg to 2800 mg, e.g., between 600 mg to 2700 mg, e.g., between 650 mg to 2600 mg, e.g., between 700 mg to 2500 mg, e.g., between 1000 mg to 2400 mg, e.g., between 1100 mg to 2300 mg, e.g., between 1200 mg to 2200 mg, e.g., between 1300 mg to 2100 mg, e.g., between 1400 mg to 2000 mg, e.g., between 1500 mg to 1900 mg, e.g., between 1600 mg to 1800 mg, e.g., between 1620 mg to 1700 mg, e.g., between 1640 mg to 1690 mg, e.g., between 1660 mg to 1680 mg, 1680 mg, e.g., 1600 mg, 1610 mg, 1620 mg, 1630 mg, 1640 mg, 1650 mg, 1660 mg, 1670 mg, 1680 mg, 1690 mg, or 1700 mg) every four weeks (Q4W). In some instances, the effective amount of the PD-1 axis binding antagonist (e.g., anti-PD-L1 antagonist antibody (e.g., atezolizumab) or anti-PD-1 antagonist antibody (e.g., pembrolizumab)) is a dose of 1680 mg every four weeks (e.g., 1680 mg±10 mg, e.g., 1680±6 mg, e.g., 1680±5 mg, e.g., 1680±3 mg, e.g., 1680±1 mg, e.g., 1680 0.5 mg, e.g., 1680 mg every four weeks). In some instances, the effective amount of atezolizumab is a dose of about 1680 mg every four weeks. In some instances, the effective amount of atezolizumab is a dose of 1680 mg every four weeks.

In some instances, the effective amount of an anti-PD-1 antagonist antibody (e.g., pembrolizumab) is a dose of between about 50 mg to about 2000 mg (e.g., between about 50-100 mg, between about 100-250 mg, between about 250-500 mg, between about 500-750 mg, between about 750-1000 mg, between about 1000-1250 mg, between about 1250-1500 mg, between about 1500-1750 mg, or between about 1750-2000 mg, e.g., between about 100 mg to about 1000 mg, between about 120 mg to about 900 mg, between about 150 mg to about 800 mg, between about 200 mg to about 700 mg, between about 250 mg to about 600 mg, between about 300 mg to about 500 mg, or between about 350 mg to about 450 mg, e.g., between about 50 mg to about 100 mg, between about 100 mg to about 200 mg, between about 200 mg to about 300 mg, between about 300 mg to about 400 mg, between about 400 mg to about 500 mg, between about 500 mg to about 600 mg, between about 600 mg to about 700 mg, between about 700 mg to about 800 mg, or between about 800 mg to about 1000 mg, e.g., about 50 mg, about 100 mg, about 250 mg, about 500 mg, about 750 mg, about 1000 mg, about 1250 mg, about 1500 mg, about 1750 mg, or about 2000 mg, e.g., about 300 mg, about 310 mg, about 320 mg, about 330 mg, about 340 mg, about 350 mg, about 360 mg, about 370 mg, about 380 mg, about 390 mg, about 400 mg, about 410 mg, about 420 mg, about 430 mg, about 440 mg, about 450 mg, about 460 mg, about 470 mg, about 480 mg, about 490 mg, or about 500 mg, e.g., 400 mg) every six weeks (Q6W). In some instances, the effective amount of an anti-PD-1 antagonist antibody (e.g., pembrolizumab) is a dose of between 50 mg to 2000 mg (e.g., between 100 mg to 1000 mg, between 120 mg to 900 mg, between 150 mg to 800 mg, between 200 mg to 700 mg, between 250 mg to 600 mg, between 300 mg to 500 mg, or between 350 mg to 450 mg, e.g., between 50 mg to 100 mg, between 100 mg to 200 mg, between 200 mg to 300 mg, between 300 mg to 400 mg, between 400 mg to 500 mg, between 500 mg to 600 mg, between 600 mg to 700 mg, between 700 mg to 800 mg, or between 800 mg to 1000 mg, e.g., 300 mg, 310 mg, 320 mg, 330 mg, 340 mg, 350 mg, 360 mg, 370 mg, 380 mg, 390 mg, 400 mg, 410 mg, 420 mg, 430 mg, 440 mg, 450 mg, 460 mg, 470 mg, 480 mg, 490 mg, or 500 mg, e.g., 400 mg) every six weeks (Q6W). In some instances, the effective amount of the anti-PD-1 antagonist antibody (e.g., pembrolizumab) is a dose of about 400 mg every six weeks (e.g., 400 mg±10 mg, e.g., 400±6 mg, e.g., 400±5 mg, e.g., 400±3 mg, e.g., 400±1 mg, e.g., 400±0.5 mg, e.g., 400 mg every six weeks). In some instances, the dose of the PD-1 axis binding antagonist is a fixed dose. In some instances, the effective amount of pembrolizumab is a dose of (e.g., a fixed dose) about 400 mg every six weeks. In some instances, the effective amount of pembrolizumab is a dose (e.g., a fixed dose) of 400 mg every six weeks.

In some instances, the dose of the PD-1 axis binding antagonist (e.g., anti-PD-L1 antagonist antibody (e.g., atezolizumab) or anti-PD-1 antagonist antibody (e.g., pembrolizumab)) administered in a combination therapy (e.g., a combination treatment with an anti-TIGIT antagonist antibody, such as an anti-TIGIT antagonist antibody disclosed herein, e.g., tiragolumab) may be reduced as compared to a standard dose of the PD-1 axis binding antagonist administered as a monotherapy.

In some instances, the PD-1 axis binding antagonist (e.g., anti-PD-L1 antagonist antibody (e.g., atezolizumab) or anti-PD-1 antagonist antibody (e.g., pembrolizumab)) is administered intravenously. Alternatively, in some embodiments, the PD-1 axis binding antagonist (e.g., anti-PD-L1 antagonist antibody (e.g., atezolizumab) or anti-PD-1 antagonist antibody (e.g., pembrolizumab)) is administered subcutaneously. In some instances, atezolizumab is administered to the patient intravenously at a dose of about 840 mg every 2 weeks, about 1200 mg every 3 weeks, or about 1680 mg of every 4 weeks. In some instances, atezolizumab is administered to the patient intravenously at a dose of 840 mg every 2 weeks, 1200 mg every 3 weeks, or 1680 mg of every 4 weeks.

In some instances, a subject is administered a total of 1 to 20 doses of a PD-1 axis binding antagonist, e.g., 1, 2, 3, 4, 5, 6, 7, 8, 9, 10, 11, 12, 13, 14, 15, 16, 17, 18, 19, or 20 doses. In some instances, a subject is administered a total of 1 to 50 doses of a PD-1 axis binding antagonist, e.g., 1 to 50 doses, 1 to 45 doses, 1 to 40 doses, 1 to 35 doses, 1 to 30 doses, 1 to 25 doses, 1 to 20 doses, 1 to 15 doses, 1 to 10 doses, 1 to 5 doses, 2 to 50 doses, 2 to 45 doses, 2 to 40 doses, 2 to 35 doses, 2 to 30 doses, 2 to 25 doses, 2 to 20 doses, 2 to 15 doses, 2 to 10 doses, 2 to 5 doses, 3 to 50 doses, 3 to 45 doses, 3 to 40 doses, 3 to 35 doses, 3 to 30 doses, 3 to 25 doses, 3 to 20 doses, 3 to 15 doses, 3 to 10 doses, 3 to 5 doses, 4 to 50 doses, 4 to 45 doses, 4 to 40 doses, 4 to 35 doses, 4 to 30 doses, 4 to 25 doses, 4 to 20 doses, 4 to 15 doses, 4 to 10 doses, 4 to 5 doses, 5 to 50 doses, 5 to 45 doses, 5 to 40 doses, 5 to 35 doses, 5 to 30 doses, 5 to 25 doses, 5 to 20 doses, 5 to 15 doses, 5 to 10 doses, 10 to 50 doses, 10 to 45 doses, 10 to 40 doses, 10 to 35 doses, 10 to 30 doses, 10 to 25 doses, 10 to 20 doses, 10 to 15 doses, 15 to 50 doses, 15 to 45 doses, 15 to 40 doses, 15 to 35 doses, 15 to 30 doses, 15 to 25 doses, 15 to 20 doses, 20 to 50 doses, 20 to 45 doses, 20 to 40 doses, 20 to 35 doses, 20 to 30 doses, 20 to 25 doses, 25 to 50 doses, 25 to 45 doses, 25 to 40 doses, 25 to 35 doses, 25 to 30 doses, 30 to 50 doses, 30 to 45 doses, 30 to 40 doses, 30 to 35 doses, 35 to 50 doses, 35 to 45 doses, 35 to 40 doses, 40 to 50 doses, 40 to 45 doses, or 45 to 50 doses. In particular instances, the doses may be administered intravenously.

iii. Effective Dosages of Anti-TIGIT Antagonist Antibodies and PD-1 Axis Binding Antagonists

In some instances, the effective amount of the anti-TIGIT antagonist antibody (e.g., an anti-TIGIT antagonist antibody as disclosed herein, e.g., tiragolumab) is a dose (e.g., a fixed dose) of between about 30 mg to about 600 mg (e.g., between about 50 mg to between 600 mg, e.g., between about 60 mg to about 600 mg, e.g., between about 100 mg to about 600 mg, e.g., between about 200 mg to about 600 mg, e.g., between about 200 mg to about 550 mg, e.g., between about 250 mg to about 600 mg, e.g., between about 300 mg to about 500 mg, e.g., between about 350 mg to about 450 mg, e.g., between about 400 mg to about 440 mg, e.g., between about 410 mg to about 430 mg, e.g., about 420±10 mg, e.g., 420±6 mg, e.g., 420±5 mg, e.g., 420±3 mg, e.g., 420±1 mg, e.g., 420±0.5 mg, e.g., 420 mg) every two weeks. In some instances, the effective amount of the anti-TIGIT antagonist antibody (e.g., an anti-TIGIT antagonist antibody as disclosed herein, e.g., tiragolumab) is a dose (e.g., a fixed dose) of between 30 mg to 600 mg (e.g., between 50 mg to between 600 mg, e.g., between 60 mg to 600 mg, e.g., between 100 mg to 600 mg, e.g., between 200 mg to 600 mg, e.g., between 200 mg to 550 mg, e.g., between 250 mg to 600 mg, e.g., between 300 mg to 500 mg, e.g., between 350 mg to 450 mg, e.g., between 400 mg to 440 mg, e.g., between 410 mg to 430 mg, e.g., 420±10 mg, e.g., 420±6 mg, e.g., 420±5 mg, e.g., 420±3 mg, e.g., 420±1 mg, e.g., 420±0.5 mg, e.g., 420 mg) every two weeks. In some instances, the effective amount of the anti-TIGIT antagonist antibody (e.g., an anti-TIGIT antagonist antibody as disclosed herein, e.g., tiragolumab) is a dose of about 420 mg every two weeks. In some instances, the effective amount of the anti-TIGIT antagonist antibody (e.g., an anti-TIGIT antagonist antibody as disclosed herein, e.g., tiragolumab) is a dose of 420 mg every two weeks.

In some instances, the effective amount of the anti-TIGIT antagonist antibody (e.g., an anti-TIGIT antagonist antibody as disclosed herein, e.g., tiragolumab) is a dose of between about 30 mg to about 1200 mg (e.g., between about 30 mg to about 1100 mg, e.g., between about 60 mg to about 1000 mg, e.g., between about 100 mg to about 900 mg, e.g., between about 200 mg to about 800 mg, e.g., between about 300 mg to about 800 mg, e.g., between about 400 mg to about 800 mg, e.g., between about 400 mg to about 750 mg, e.g., between about 450 mg to about 750 mg, e.g., between about 500 mg to about 700 mg, e.g., between about 550 mg to about 650 mg, e.g., 600 mg±10 mg, e.g., 600±6 mg, e.g., 600±5 mg, e.g., 600±3 mg, e.g., 600±1 mg, e.g., 600±0.5 mg, e.g., 600 mg) every three weeks. In some instances, the effective amount of the anti-TIGIT antagonist antibody (e.g., an anti-TIGIT antagonist antibody as disclosed herein, e.g., tiragolumab) is a dose of between 30 mg to 1200 mg (e.g., between 30 mg to 1100 mg, e.g., between 60 mg to 1000 mg, e.g., between 100 mg to 900 mg, e.g., between 200 mg to 800 mg, e.g., between 300 mg to 800 mg, e.g., between 400 mg to 800 mg, e.g., between 400 mg to 750 mg, e.g., between 450 mg to 750 mg, e.g., between 500 mg to 700 mg, e.g., between 550 mg to 650 mg, e.g., 600 mg±10 mg, e.g., 600±6 mg, e.g., 600±5 mg, e.g., 600±3 mg, e.g., 600±1 mg, e.g., 600±0.5 mg, e.g., 600 mg) every three weeks. In some instances, the effective amount of the anti-TIGIT antagonist antibody (e.g., an anti-TIGIT antagonist antibody as disclosed herein, e.g., tiragolumab) is a dose of about 600 mg every three weeks. In some instances, the effective amount of the anti-TIGIT antagonist antibody (e.g., an anti-TIGIT antagonist antibody as disclosed herein, e.g., tiragolumab) is a dose of 600 mg every three weeks.

In some instances, the effective amount of the anti-TIGIT antagonist antibody (e.g., an anti-TIGIT antagonist antibody as disclosed herein, e.g., tiragolumab) is a dose of between about 600 mg to about 1200 mg (e.g., between about 600 mg to about 1100 mg, e.g., between about 600 mg to about 1000 mg, e.g., between about 700 mg to about 950 mg, e.g., between about 800 mg to about 900 mg, e.g., between about 820 mg to about 860 mg, e.g., about 840±10 mg, e.g., 840±6 mg, e.g., 840±5 mg, e.g., 840±3 mg, e.g., 840±1 mg, e.g., 840±0.5 mg, e.g., 840 mg) every four weeks. In some instances, the effective amount of the anti-TIGIT antagonist antibody (e.g., an anti-TIGIT antagonist antibody as disclosed herein, e.g., tiragolumab) is a dose of between 600 mg to 1200 mg (e.g., between 600 mg to 1100 mg, e.g., between 600 mg to 1000 mg, e.g., between 700 mg to 950 mg, e.g., between 800 mg to 900 mg, e.g., between 820 mg to 860 mg, e.g., 840±10 mg, e.g., 840±6 mg, e.g., 840±5 mg, e.g., 840±3 mg, e.g., 840±1 mg, e.g., 840±0.5 mg, e.g., 840 mg) every four weeks. In some instances, the effective amount of the anti-TIGIT antagonist antibody (e.g., an anti-TIGIT antagonist antibody as disclosed herein, e.g., tiragolumab) is a dose of about 840 mg every four weeks. In some instances, the effective amount of the anti-TIGIT antagonist antibody (e.g., an anti-TIGIT antagonist antibody as disclosed herein, e.g., tiragolumab) is a dose of 840 mg every four weeks.

In some instances, the dose of the anti-TIGIT antagonist antibody (e.g., an anti-TIGIT antagonist antibody as disclosed herein, e.g., tiragolumab) administered in a combination therapy (e.g., a combination treatment with a PD-1 axis binding antagonist (e.g., an anti-PD-L1 antagonist antibody (e.g., atezolizumab) or an anti-PD-1 antagonist antibody (e.g., MDX-1106 (nivolumab) or MK-3475 (pembrolizumab, previously known as lambrolizumab))) may be reduced as compared to a standard dose of the anti-TIGIT antagonist antibody administered as a monotherapy. In some instances, the dose of the anti-TIGIT antagonist antibody (e.g., an anti-TIGIT antagonist antibody as disclosed herein, e.g., tiragolumab) administered in a combination therapy (e.g., a combination treatment with a PD-1 axis binding antagonist (e.g., anti-PD-L1 antagonist antibody (e.g., atezolizumab)), with or without one or more chemotherapeutic agents (e.g., a platinum-based chemotherapeutic agent (e.g., carboplatin or cisplatin) and/or a non-platinum-based chemotherapeutic agent (e.g., an alkylating agent (e.g., cyclophosphamide), a taxane (e.g., paclitaxel or nab-paclitaxel), and/or a topoisomerase II inhibitor (e.g., doxorubicin))) and/or G-CSF or GM-CSF, may be reduced as compared to a standard dose of the anti-TIGIT antagonist antibody administered as a monotherapy.

In some instances, the effective amount of the PD-1 axis binding antagonist (e.g., anti-PD-L1 antagonist antibody (e.g., atezolizumab)) is a dose of between about 20 mg to about 1600 mg (e.g., between about 20-100 mg, between about 100-200 mg, between about 200-400 mg, between about 400-600 mg, between about 600-800 mg, between about 800-1000 mg, between about 1000-1200 mg, between about 1200-1400 mg, or between about 1400-1600 mg, e.g., between about 80 mg to about 1200 mg, e.g., between about 100 mg to about 1200 mg, e.g., between about 200 mg to about 1200 mg, e.g., between about 300 mg to about 1200 mg, e.g., between about 400 mg to about 1200 mg, e.g., between about 500 mg to about 1200 mg, e.g., between about 600 mg to about 1100 mg, e.g., between about 700 mg to about 1000 mg, e.g., between about 740 mg to about 940 mg, e.g., between about 790 mg to about 890 mg, e.g., between about 815 mg to about 865 mg, e.g., between about 830 mg to about 850 mg, e.g., 840 mg±5 mg, e.g., 840±2.5 mg, e.g., 840±1.0 mg, e.g., 840±0.5 mg, e.g., 840 mg) every two weeks. In some instances, the effective amount of the PD-1 axis binding antagonist (e.g., anti-PD-L1 antagonist antibody (e.g., atezolizumab)) is a dose of between 80 mg to 1200 mg (e.g., between 80 mg to 1200 mg, e.g., between 100 mg to 1200 mg, e.g., between 200 mg to 1200 mg, e.g., between 300 mg to 1200 mg, e.g., between 400 mg to 1200 mg, e.g., between 500 mg to 1200 mg, e.g., between 600 mg to 1100 mg, e.g., between 700 mg to 1000 mg, e.g., between 740 mg to 940 mg, e.g., between 790 mg to 890 mg, e.g., between 815 mg to 865 mg, e.g., between 830 mg to 850 mg, e.g., 840 mg±5 mg, e.g., 840±2.5 mg, e.g., 840±1.0 mg, e.g., 840±0.5 mg, e.g., 840 mg) every two weeks. In some instances, the effective amount of the PD-1 axis binding antagonist (e.g., anti-PD-L1 antagonist antibody (e.g., atezolizumab)) is a dose of about 20 mg, 100 mg, 200 mg, 300 mg, 400 mg, 500 mg, 600 mg, 700 mg, 800 mg, 900 mg, 1000 mg, 1200 mg, 1400 mg, or 1600 mg every two weeks. In some instances, the effective amount of the PD-1 axis binding antagonist (e.g., anti-PD-L1 antagonist antibody (e.g., atezolizumab)) is a dose of about 840 mg every two weeks. In some instances, the effective amount of the PD-1 axis binding antagonist (e.g., anti-PD-L1 antagonist antibody (e.g., atezolizumab)) is a dose of 840 mg every two weeks.

In some instances, the effective amount of the PD-1 axis binding antagonist (e.g., anti-PD-L1 antagonist antibody (e.g., atezolizumab)) is a dose of between about 80 mg to about 2000 mg (e.g., between about 100 mg to about 2000 mg, e.g., between about 200 mg to about 1900 mg, e.g., between about 300 mg to about 1700 mg, e.g., between about 400 mg to about 1600 mg, e.g., between about 500 mg to about 1600 mg, e.g., between about 600 mg to about 1600 mg, e.g., between about 700 mg to about 1600 mg, e.g., between about 800 mg to about 1600 mg, e.g., between about 900 mg to about 1500 mg, e.g., between about 1000 mg to about 1400 mg, e.g., between about 1050 mg to about 1350 mg, e.g., between about 1100 mg to about 1300 mg, e.g., between about 1150 mg to about 1250 mg, e.g., between about 1175 mg to about 1225 mg, e.g., between about 1190 mg to about 1210 mg, e.g., 1200 mg±5 mg, e.g., 1200±2.5 mg, e.g., 1200±1.0 mg, e.g., 1200±0.5 mg, e.g., 1200 mg) every three weeks. In some instances, the effective amount of the PD-1 axis binding antagonist (e.g., anti-PD-L1 antagonist antibody (e.g., atezolizumab)) is a dose of between 80 mg to 2000 mg (e.g., between 100 mg to 2000 mg, e.g., between 200 mg to 1900 mg, e.g., between 300 mg to 1700 mg, e.g., between 400 mg to 1600 mg, e.g., between 500 mg to 1600 mg, e.g., between 600 mg to 1600 mg, e.g., between 700 mg to 1600 mg, e.g., between 800 mg to 1600 mg, e.g., between 900 mg to 1500 mg, e.g., between 1000 mg to 1400 mg, e.g., between 1050 mg to 1350 mg, e.g., between 1100 mg to 1300 mg, e.g., between 1150 mg to 1250 mg, e.g., between 1175 mg to 1225 mg, e.g., between 1190 mg to 1210 mg, e.g., 1200 mg±5 mg, e.g., 1200±2.5 mg, e.g., 1200±1.0 mg, e.g., 1200±0.5 mg, e.g., 1200 mg) every three weeks. In some instances, the effective amount of the PD-1 axis binding antagonist (e.g., anti-PD-L1 antagonist antibody (e.g., atezolizumab)) is a dose of about 1200 mg every three weeks. In some instances, the effective amount of the PD-1 axis binding antagonist (e.g., anti-PD-L1 antagonist antibody (e.g., atezolizumab)) is a dose of 1200 mg every three weeks.

In some instances, the effective amount of the PD-1 axis binding antagonist (e.g., anti-PD-L1 antagonist antibody (e.g., atezolizumab)) is a dose of between about 1200 mg to about 2000 mg (e.g., between about 1200 mg to about 1900 mg, e.g., between about 1200 mg to about 1800 mg, e.g., between about 1300 mg to about 1800 mg, e.g., between about 1400 mg to about 1800 mg, e.g., between about 1500 mg to about 1800 mg, e.g., between about 1580 mg to about 1780 mg, e.g., between about 1630 mg to about 1730 mg, e.g., between about 1655 mg to about 1705 mg, e.g., between about 1670 mg to about 1690 mg, e.g., 1680 mg±5 mg, e.g., 1680±2.5 mg, e.g., 1680±1.0 mg, e.g., 1680±0.5 mg, e.g., 1680 mg) every four weeks. In some instances, the effective amount of the PD-1 axis binding antagonist (e.g., anti-PD-L1 antagonist antibody (e.g., atezolizumab)) is a dose of between 1200 mg to 2000 mg (e.g., between 1200 mg to 1900 mg, e.g., between 1200 mg to 1800 mg, e.g., between 1300 mg to 1800 mg, e.g., between 1400 mg to 1800 mg, e.g., between 1500 mg to 1800 mg, e.g., between 1580 mg to 1780 mg, e.g., between 1630 mg to 1730 mg, e.g., between 1655 mg to 1705 mg, e.g., between 1670 mg to 1690 mg, e.g., 1680 mg±5 mg, e.g., 1680±2.5 mg, e.g., 1680±1.0 mg, e.g., 1680±0.5 mg, e.g., 1680 mg) every four weeks. In some instances, the effective amount of the PD-1 axis binding antagonist (e.g., anti-PD-L1 antagonist antibody (e.g., atezolizumab)) is a dose of about 1680 mg every four weeks. In some instances, the effective amount of the PD-1 axis binding antagonist (e.g., anti-PD-L1 antagonist antibody (e.g., atezolizumab)) is a dose of 1680 mg every four weeks.

In some instances, the dose of the PD-1 axis binding antagonist (e.g., anti-PD-L1 antagonist antibody (e.g., atezolizumab)) administered in a combination therapy (e.g., a combination treatment with an anti-TIGIT antagonist antibody, such as an anti-TIGIT antagonist antibody disclosed herein (e.g., tiragolumab) may be reduced as compared to a standard dose of the anti-PD-L1 antagonist antibody administered as a monotherapy. In some instances, the dose of the PD-1 axis binding antagonist (e.g., anti-PD-L1 antagonist antibody (e.g., atezolizumab)) administered in a combination therapy (e.g., a combination treatment with an anti-TIGIT antagonist antibody, such as an anti-TIGIT antagonist antibody disclosed herein, e.g., tiragolumab), with or without one or more chemotherapeutic agents (e.g., a platinum-based chemotherapeutic agent (e.g., carboplatin or cisplatin) and/or a non-platinum-based chemotherapeutic agent (e.g., an alkylating agent (e.g., cyclophosphamide), a taxane (e.g., paclitaxel, e.g., nab-paclitaxel), and/or a topoisomerase II inhibitor (e.g., doxorubicin))) and/or G-CSF or GM-CSF may be reduced as compared to a standard dose of the PD-1 axis binding antagonist administered as a monotherapy.

iv. Dosing Cycles for Anti-TIGIT Antagonist Antibodies and PD-1 Axis Binding Antagonists

In any of the methods and uses of the invention, the anti-TIGIT antagonist antibody (e.g., an anti-TIGIT antagonist antibody as disclosed herein, e.g., tiragolumab) and/or the PD-1 axis binding antagonist (e.g., anti-PD-L1 antagonist antibody (e.g., atezolizumab)) may be administered in one or more dosing cycles (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 or more dosing cycles). In some instances, the dosing cycles of the anti-TIGIT antagonist antibody (e.g., an anti-TIGIT antagonist antibody as disclosed herein, e.g., tiragolumab) and the PD-1 axis binding antagonist (e.g., anti-PD-L1 antagonist antibody (e.g., atezolizumab)) continue until there is a loss of clinical benefit (e.g., confirmed disease progression, drug resistance, death, or unacceptable toxicity). In some instances, the length of each dosing cycle is about 7 to 42 days (e.g., 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 31 days, 32 days, 33 days, 34 days, 35 days, 36 days, 37 days, 38 days, 39 days, 41 days, 42 days). In some instances, the length of each dosing cycle is about 14 days. In some instances, the length of each dosing cycle is about 21 days. In some instances, the length of each dosing cycle is about 28 days. In some instances, the length of each dosing cycle is about 42 days. In some instances, the length of each dosing cycle is about 7 days. In some instances, the anti-TIGIT antagonist antibody (e.g., an anti-TIGIT antagonist antibody as disclosed herein, e.g., tiragolumab) is administered on about Day 1 (e.g., Day 1±3 days) of each dosing cycle. In some instances, the anti-TIGIT antagonist antibody (e.g., an anti-TIGIT antagonist antibody as disclosed herein, e.g., tiragolumab) is administered on about Day 15 (e.g., Day 15±3 days) of each dosing cycle. In some instances, the anti-TIGIT antagonist antibody (e.g., an anti-TIGIT antagonist antibody as disclosed herein, e.g., tiragolumab) is administered on about Day 22 (e.g., Day 22±3 days) of each dosing cycle. In some instances, the anti-TIGIT antagonist antibody (e.g., an anti-TIGIT antagonist antibody as disclosed herein, e.g., tiragolumab) is administered on about Day 29 (e.g., Day 29±3 days) of each dosing cycle. For example, the anti-TIGIT antagonist antibody (e.g., an anti-TIGIT antagonist antibody as disclosed herein, e.g., tiragolumab) is administered intravenously at a dose (e.g., a fixed dose) of about 600 mg on Day 1 of each 21-day cycle (i.e., at a dose of about 600 mg every three weeks). For example, the anti-TIGIT antagonist antibody (e.g., an anti-TIGIT antagonist antibody as disclosed herein, e.g., tiragolumab) is administered intravenously at a dose (e.g., a fixed dose) of about 600 mg on Day 1 and Day 15 of each 28-day cycle (i.e., at a dose of about 420 mg every two weeks). For example, the anti-TIGIT antagonist antibody (e.g., an anti-TIGIT antagonist antibody as disclosed herein, e.g., tiragolumab) is administered intravenously at a dose (e.g., a fixed dose) of about 600 mg on Day 1, Day 15, and Day 29 of each 42-day cycle (i.e., at a dose of about 420 mg every two weeks). For example, the anti-TIGIT antagonist antibody (e.g., an anti-TIGIT antagonist antibody as disclosed herein, e.g., tiragolumab) is administered intravenously at a dose (e.g., a fixed dose) of about 600 mg on Day 1 and Day 22 of each 42-day cycle (i.e., at a dose of about 600 mg every three weeks). In some instances, the PD-1 axis binding antagonist (e.g., an anti-PD-L1 antagonist antibody (e.g., atezolizumab) or an anti-PD-1 antagonist antibody (e.g., MDX-1106 (nivolumab) or MK-3475 (pembrolizumab, previously known as lambrolizumab))) is administered on about Day 1 (e.g., Day 1±3 days) of each dosing cycle. In some instances, the PD-1 axis binding antagonist (e.g., an anti-PD-L1 antagonist antibody (e.g., atezolizumab) or an anti-PD-1 antagonist antibody (e.g., MDX-1106 (nivolumab) or MK-3475 (pembrolizumab, previously known as lambrolizumab))) is administered on about Day 15 (e.g., Day 15±3 days) of each dosing cycle. For example, the PD-1 axis binding antagonist (e.g., anti-PD-L1 antagonist antibody (e.g., atezolizumab)) is administered intravenously at a dose of about 1200 mg on Day 1 of each 21-day cycle (i.e., at a dose of about 1200 mg every three weeks). For example, the PD-1 axis binding antagonist (e.g., anti-PD-L1 antagonist antibody (e.g., atezolizumab)) is administered intravenously at a dose of about 1200 mg on Day 1 and Day 15 of each 28-day cycle (i.e., at a dose of about 840 mg every two weeks). In some examples, the anti-TIGIT antagonist antibody (e.g., an anti-TIGIT antagonist antibody as disclosed herein, e.g., tiragolumab) is administered intravenously at a dose (e.g., a fixed dose) of 600 mg on Day 1 of each 21-day cycle (i.e., at a dose of 600 mg every three weeks). In some instances, the PD-1 axis binding antagonist (e.g., an anti-PD-L1 antagonist antibody (e.g., atezolizumab) or an anti-PD-1 antagonist antibody (e.g., MDX-1106 (nivolumab) or MK-3475 (pembrolizumab, previously known as lambrolizumab))) is administered on Day 1 (e.g., Day 1±3 days) of each dosing cycle. For example, the PD-1 axis binding antagonist (e.g., anti-PD-L1 antagonist antibody (e.g., atezolizumab)) is administered intravenously at a dose of 1200 mg on Day 1 of each 21-day cycle (i.e., at a dose of 1200 mg every three weeks).

In some instances, the anti-TIGIT antagonist antibody (e.g., an anti-TIGIT antagonist antibody as disclosed herein, e.g., tiragolumab) and the PD-1 axis binding antagonist (e.g., anti-PD-L1 antagonist antibody (e.g., atezolizumab)) are administered on about Day 1 (e.g., Day 1±3 days) of each dosing cycle.

In some instances, the anti-TIGIT antagonist antibody (e.g., an anti-TIGIT antagonist antibody as disclosed herein, e.g., tiragolumab) is administered intravenously at a dose of about 600 mg on Day 1 of each 21-day cycle (i.e., at a dose of about 600 mg every three weeks) and the PD-1 axis binding antagonist (e.g., anti-PD-L1 antagonist antibody (e.g., atezolizumab)) is administered intravenously at a dose of about 1200 mg on Day 1 of each 21-day cycle (i.e., at a dose of about 1200 mg every three weeks). In some instances, the anti-TIGIT antagonist antibody (e.g., an anti-TIGIT antagonist antibody as disclosed herein, e.g., tiragolumab) is administered intravenously at a dose of 600 mg on Day 1 of each 21-day cycle (i.e., at a dose of 600 mg every three weeks) and the PD-1 axis binding antagonist (e.g., anti-PD-L1 antagonist antibody (e.g., atezolizumab)) is administered intravenously at a dose of 1200 mg on Day 1 of each 21-day cycle (i.e., at a dose of 1200 mg every three weeks).

In other instances, the anti-TIGIT antagonist antibody (e.g., an anti-TIGIT antagonist antibody as disclosed herein, e.g., tiragolumab) is administered intravenously at a dose of about 420 mg on Day 1 of each 14-day cycle (i.e., at a dose of about 420 mg every two weeks) and the PD-1 axis binding antagonist (e.g., anti-PD-L1 antagonist antibody (e.g., atezolizumab)) is administered intravenously at a dose of about 840 mg on Day 1 of each 14-day cycle (i.e., at a dose of about 840 mg every two weeks). In some instances, the anti-TIGIT antagonist antibody (e.g., an anti-TIGIT antagonist antibody as disclosed herein, e.g., tiragolumab) is administered intravenously at a dose of 420 mg on Day 1 of each 14-day cycle (i.e., at a dose of about 420 mg every two weeks) and the PD-1 axis binding antagonist (e.g., anti-PD-L1 antagonist antibody (e.g., atezolizumab)) is administered intravenously at a dose of 840 mg on Day 1 of each 14-day cycle (i.e., at a dose of 840 mg every two weeks).

In other instances, the anti-TIGIT antagonist antibody (e.g., an anti-TIGIT antagonist antibody as disclosed herein, e.g., tiragolumab) is administered intravenously at a dose of about 840 mg on Day 1 of each 28-day cycle (i.e., at a dose of about 840 mg every four weeks) and the PD-1 axis binding antagonist (e.g., anti-PD-L1 antagonist antibody (e.g., atezolizumab)) is administered intravenously at a dose of about 1680 mg on Day 1 of each 28-day cycle (i.e., at a dose of about 1680 mg every four weeks). In some instances, the anti-TIGIT antagonist antibody (e.g., an anti-TIGIT antagonist antibody as disclosed herein, e.g., tiragolumab) is administered intravenously at a dose of 840 mg on Day 1 of each 28-day cycle (i.e., at a dose of 840 mg every four weeks) and the PD-1 axis binding antagonist (e.g., anti-PD-L1 antagonist antibody (e.g., atezolizumab)) is administered intravenously at a dose of 1680 mg on Day 1 of each 28-day cycle (i.e., at a dose of 1680 mg every four weeks).

v. Intravenous Infusion and Subcutaneous Administration of Anti-TIGIT Antagonist Antibodies and PD-1 Axis Binding Antagonists

In some instances, the anti-TIGIT antagonist antibody (e.g., an anti-TIGIT antagonist antibody as disclosed herein, e.g., tiragolumab) is administered intravenously. Alternatively, in some embodiments, the anti-TIGIT antagonist antibody (e.g., an anti-TIGIT antagonist antibody as disclosed herein, e.g., tiragolumab) is administered subcutaneously. In some instances, the PD-1 axis binding antagonist (e.g., anti-PD-L1 antagonist antibody (e.g., atezolizumab)) is administered intravenously. Alternatively, in some embodiments, the PD-1 axis binding antagonist (e.g., anti-PD-L1 antagonist antibody (e.g., atezolizumab)) is administered subcutaneously.

In some instances, the anti-TIGIT antagonist antibody (e.g., an anti-TIGIT antagonist antibody as disclosed herein, e.g., tiragolumab) is administered to the subject or population of subjects by intravenous infusion over about 60±15 minutes (e.g., about 45 minutes, about 46 minutes, about 47 minutes, about 48 minutes, about 49 minutes, about 50 minutes, about 51 minutes, about 52 minutes, about 53 minutes, about 54 minutes, about 55 minutes, about 56 minutes, about 57 minutes, about 58 minutes, about 59 minutes, about 60 minutes, about 61 minutes, about 62 minutes, about 63 minutes, about 64 minutes, about 65 minutes, about 66 minutes, about 67 minutes, about 68 minutes, about 69 minutes, about 70 minutes, about 71 minutes, about 72 minutes, about 73 minutes, about 74 minutes, or about 75 minutes). In some instances, the anti-TIGIT antagonist antibody (e.g., an anti-TIGIT antagonist antibody as disclosed herein, e.g., tiragolumab) is administered to the subject or population of subjects by intravenous infusion over about 60±10 minutes (e.g., about 50 minutes, about 51 minutes, about 52 minutes, about 53 minutes, about 54 minutes, about 55 minutes, about 56 minutes, about 57 minutes, about 58 minutes, about 59 minutes, about 60 minutes, about 61 minutes, about 62 minutes, about 63 minutes, about 64 minutes, about 65 minutes, about 66 minutes, about 67 minutes, about 68 minutes, about 69 minutes, or about 70 minutes). In some instances, the PD-1 axis binding antagonist (e.g., anti-PD-L1 antagonist antibody (e.g., atezolizumab) is administered to the subject by intravenous infusion over about 60±15 minutes (e.g., about 45 minutes, about 46 minutes, about 47 minutes, about 48 minutes, about 49 minutes, about 50 minutes, about 51 minutes, about 52 minutes, about 53 minutes, about 54 minutes, about 55 minutes, about 56 minutes, about 57 minutes, about 58 minutes, about 59 minutes, about 60 minutes, about 61 minutes, about 62 minutes, about 63 minutes, about 64 minutes, about 65 minutes, about 66 minutes, about 67 minutes, about 68 minutes, about 69 minutes, about 70 minutes, about 71 minutes, about 72 minutes, about 73 minutes, about 74 minutes, or about 75 minutes).

In some instances, the anti-TIGIT antagonist antibody (e.g., an anti-TIGIT antagonist antibody as disclosed herein, e.g., tiragolumab) is administered to the subject by intravenous infusion over about 30+10 minutes (e.g., about 20 minutes, about 21 minutes, about 22 minutes, about 23 minutes, about 24 minutes, about 25 minutes, about 26 minutes, about 27 minutes, about 28 minutes, about 29 minutes, about 30 minutes, about 31 minutes, about 32 minutes, about 33 minutes, about 34 minutes, about 35 minutes, about 36 minutes, about 37 minutes, about 38 minutes, about 39 minutes, or about 40 minutes). In some instances, the PD-1 axis binding antagonist (e.g., anti-PD-L1 antagonist antibody (e.g., atezolizumab)) is administered to the subject by intravenous infusion over about 30±10 minutes (e.g., about 20 minutes, about 21 minutes, about 22 minutes, about 23 minutes, about 24 minutes, about 25 minutes, about 26 minutes, about 27 minutes, about 28 minutes, about 29 minutes, about 30 minutes, about 31 minutes, about 32 minutes, about 33 minutes, about 34 minutes, about 35 minutes, about 36 minutes, about 37 minutes, about 38 minutes, about 39 minutes, or about 40 minutes).

vi. Administration Order and Observation Periods

In some instances in which both an anti-TIGIT antagonist antibody and PD-1 axis binding antagonist are administered to a subject or population of subjects, the anti-TIGIT antagonist antibody (e.g., an anti-TIGIT antagonist antibody as disclosed herein, e.g., tiragolumab) is administered to the subject before the PD-1 axis binding antagonist (e.g., anti-PD-L1 antagonist antibody (e.g., atezolizumab) or anti-PD-1 antagonist antibody (e.g., pembrolizumab)).

In some instances, for example, following administration of the anti-TIGIT antagonist antibody and before administration of the PD-1 axis binding antagonist the method includes an intervening first observation period. In some instances, for example, following administration of the anti-TIGIT antagonist antibody, the PD-1 axis binding antagonist (e.g., anti-PD-L1 antagonist antibody (e.g., atezolizumab)) is administered to the subject. In some instances, the anti-TIGIT antagonist antibody (e.g., an anti-TIGIT antagonist antibody as disclosed herein, e.g., tiragolumab) is first administered to the subject and the PD-1 axis binding antagonist (e.g., anti-PD-L1 antagonist antibody (e.g., atezolizumab)) is administered to the subject following administration of the anti-TIGIT antagonist antibody (e.g., an anti-TIGIT antagonist antibody as disclosed herein, e.g., tiragolumab).

In some instances, the method further includes a second observation period following administration of the PD-1 axis binding antagonist (e.g., anti-PD-L1 antagonist antibody (e.g., atezolizumab)).

In some instances, the method includes both a first observation period following administration of the anti-TIGIT antagonist antibody and second observation period following administration of the PD-1 axis binding antagonist (e.g., anti-PD-L1 antagonist antibody (e.g., atezolizumab)). In some instances, the first and second observation periods are each between about 30 minutes to about 60 minutes in length. In instances in which the first and second observation periods are each about 60 minutes in length, the method may include recording the subject's vital signs (e.g., pulse rate, respiratory rate, blood pressure, and temperature) at about 30±10 minutes after administration of the anti-TIGIT antagonist antibody, the PD-1 axis binding antagonist (e.g., anti-PD-L1 antagonist antibody (e.g., atezolizumab)) during the first or second observation periods. In instances in which the first and second observation periods are each about 30 minutes in length, the method may include recording the subject's vital signs (e.g., pulse rate, respiratory rate, blood pressure, and temperature) at about 15±10 minutes after administration of the anti-TIGIT antagonist antibody or the PD-1 axis binding antagonist (e.g., anti-PD-L1 antagonist antibody (e.g., atezolizumab)) during the first or second.

In some instances, the PD-1 axis binding antagonist (e.g., anti-PD-L1 antagonist antibody (e.g., atezolizumab) or anti-PD-1 antagonist antibody (e.g., pembrolizumab)) is administered to the subject or population of subjects before the anti-TIGIT antagonist antibody (e.g., an anti-TIGIT antagonist antibody as disclosed herein, e.g., tiragolumab). In some instances, for example, following administration of the PD-1 axis binding antagonist (e.g., anti-PD-L1 antagonist antibody (e.g., atezolizumab)) and before administration of the anti-TIGIT antagonist antibody (e.g., an anti-TIGIT antagonist antibody as disclosed herein, e.g., tiragolumab), the method includes an intervening first observation period.

In some instances, the method further includes a second observation period following administration of the anti-TIGIT antagonist antibody (e.g., an anti-TIGIT antagonist antibody as disclosed herein, e.g., tiragolumab).

In some instances, the method includes both a first observation period following administration of the PD-1 axis binding antagonist (e.g., anti-PD-L1 antagonist antibody (e.g., atezolizumab)) and second observation period following administration of the anti-TIGIT antagonist antibody (e.g., an anti-TIGIT antagonist antibody as disclosed herein, e.g., tiragolumab). In some instances, the first and second observation periods are each between about 30 minutes to about 60 minutes in length. In instances in which the first and second observation periods are each about 60 minutes in length, the method may include recording the subject's vital signs (e.g., pulse rate, respiratory rate, blood pressure, and temperature) at about 30±10 minutes after administration of the PD-1 axis binding antagonist (e.g., anti-PD-L1 antagonist antibody (e.g., atezolizumab)) or the anti-TIGIT antagonist antibody (e.g., an anti-TIGIT antagonist antibody as disclosed herein, e.g., tiragolumab), during the first or second observation periods. In instances in which the first and second observation periods are each about 30 minutes in length, the method may include recording the subject's vital signs (e.g., pulse rate, respiratory rate, blood pressure, and temperature) at about 15±10 minutes after administration of the PD-1 axis binding antagonist (e.g., anti-PD-L1 antagonist antibody (e.g., atezolizumab)), the anti-TIGIT antagonist antibody (e.g., an anti-TIGIT antagonist antibody as disclosed herein, e.g., tiragolumab), during the first or second observation periods.

In some instances, the method further includes administration of a VEGF antagonist (e.g., an anti-VEGF antibody (e.g., bevacizumab)). In some instances, the VEGF antagonist (e.g., an anti-VEGF antibody (e.g., bevacizumab)) is administered after the PD-1 axis binding antagonist (e.g., anti-PD-L1 antagonist antibody (e.g., atezolizumab) or anti-PD-1 antagonist antibody (e.g., pembrolizumab)) and the anti-TIGIT antagonist antibody (e.g., an anti-TIGIT antagonist antibody as disclosed herein, e.g., tiragolumab). In some instances, the VEGF antagonist (e.g., an anti-VEGF antibody (e.g., bevacizumab)) is administered after the second observation period following administration of the anti-TIGIT antagonist antibody (e.g., an anti-TIGIT antagonist antibody as disclosed herein, e.g., tiragolumab) or the PD-1 axis binding antagonist (e.g., anti-PD-L1 antagonist antibody (e.g., atezolizumab) or anti-PD-1 antagonist antibody (e.g., pembrolizumab)).

In some instances, the method further includes a third observation period following administration of the VEGF antagonist (e.g., an anti-VEGF antibody (e.g., bevacizumab)). In some instances, the third observation period es between about 30 minutes to about 120 minutes in length. In some instances, the first observation period, the second observation period, and the third observation period are each between about 30 minutes to about 120 minutes in length.

Combination Dosing of Anti-TIGIT Antagonist Antibodies and PD-1 Axis Binding Antagonists

In some instances, a dose of an effective amount of an anti-TIGIT antagonist antibody (e.g., an anti-TIGIT antagonist antibody as disclosed herein, e.g., tiragolumab) is administered with a dose of an effective amount of a PD-1 axis binding antagonist (e.g., anti-PD-L1 antagonist antibody (e.g., atezolizumab)) in a combination therapy (e.g., a combination treatment of an anti-TIGIT antagonist antibody (e.g., an anti-TIGIT antagonist antibody as disclosed herein, e.g., tiragolumab) with a PD-1 axis binding antagonist (e.g., anti-PD-L1 antagonist antibody (e.g., atezolizumab)), e.g., for treatment of a subject having a cancer. In some instances, an anti-TIGIT antagonist antibody is administered every two weeks as described in Section III(K)(i) herein and a PD-1 axis binding antagonist is administered every two weeks as described in Section III(K)(ii) herein. In some instances, an anti-TIGIT antagonist antibody is administered every two weeks as described in Section III(K)(i) herein and a PD-1 axis binding antagonist is administered every three weeks as described in Section III(K)(ii) herein. In some instances, an anti-TIGIT antagonist antibody is administered every two weeks as described in Section III(K)(i) herein and a PD-1 axis binding antagonist is administered every four weeks as described in Section III(K)(ii) herein. In some instances, an anti-TIGIT antagonist antibody is administered every two weeks as described in Section III(K)(i) herein and a PD-1 axis binding antagonist is administered every six weeks as described in Section III(K)(ii) herein. In some instances, an anti-TIGIT antagonist antibody is administered every three weeks as described in Section III(K)(i) herein and a PD-1 axis binding antagonist is administered every two weeks as described in Section III(K)(ii) herein. In some instances, an anti-TIGIT antagonist antibody is administered every three weeks as described in Section III(K)(i) herein and a PD-1 axis binding antagonist is administered every three weeks as described in Section III(K)(ii) herein. In some instances, an anti-TIGIT antagonist antibody is administered every three weeks as described in Section III(K)(i) herein and a PD-1 axis binding antagonist is administered every four weeks as described in Section III(K)(ii) herein. In some instances, an anti-TIGIT antagonist antibody is administered every three weeks as described in Section III(K)(i) herein and a PD-1 axis binding antagonist is administered every six weeks as described in Section III(K)(ii) herein. In some instances, an anti-TIGIT antagonist antibody is administered every four weeks as described in Section III(K)(i) herein and a PD-1 axis binding antagonist is administered every two weeks as described in Section III(K)(ii) herein. In some instances, an anti-TIGIT antagonist antibody is administered every four weeks as described in Section III(K)(i) herein and a PD-1 axis binding antagonist is administered every three weeks as described in Section III(K)(ii) herein. In some instances, an anti-TIGIT antagonist antibody is administered every four weeks as described in Section III(K)(i) herein and a PD-1 axis binding antagonist is administered every four weeks as described in Section III(K)(ii) herein. In some instances, an anti-TIGIT antagonist antibody is administered every four weeks as described in Section III(K)(i) herein and a PD-1 axis binding antagonist is administered every six weeks as described in Section III(K)(ii) herein. In some instances, an anti-TIGIT antagonist antibody is administered every two, three, or four weeks as described in Section III(K)(i) herein and a PD-1 axis binding antagonist is administered every two, three, four, or six weeks as described in Section III(K)(ii) herein.

In some instances, the dose of the anti-TIGIT antagonist antibody (e.g., an anti-TIGIT antagonist antibody as disclosed herein, e.g., tiragolumab) is a dose of about 600 mg every three weeks. In some instances, the dose of the anti-TIGIT antagonist antibody (e.g., an anti-TIGIT antagonist antibody as disclosed herein, e.g., tiragolumab) is a dose of 600 mg every three weeks. In some instances, the anti-TIGIT antagonist antibody (e.g., an anti-TIGIT antagonist antibody as disclosed herein, e.g., tiragolumab) is administered (e.g., every three weeks) in a tiered dosing regimen (e.g., dosing based on body weight (BW) or body surface area (BSA) of a subject) and a PD-1 axis binding antagonist (e.g., an anti-PD-L1 antagonist antibody, such as atezolizumab) at a dose from about 0.01 mg/kg to about 50 mg/kg (e.g., about 15 mg/kg) up to 1200 mg, e.g., every three weeks. In some instances, the anti-TIGIT antagonist antibody (e.g., an anti-TIGIT antagonist antibody as disclosed herein, e.g., tiragolumab) is administered (e.g., every three weeks) in a tiered dosing regimen (e.g., dosing based on body weight (BW) or body surface area (BSA) of a subject) and a PD-1 axis binding antagonist (e.g., an anti-PD-L1 antagonist antibody, such as atezolizumab) at a dose from 0.01 mg/kg to 50 mg/kg (e.g., 15 mg/kg) up to 1200 mg, e.g., every three weeks. Such dosing regimens can be utilized in treatments for subjects having relatively low body weight (e.g., 40 kg or less (e.g., from 5 kg to 40 kg, from 15 kg to 40 kg, or from 5 kg to 15 kg)) and have been developed through biosimulation studies based on extrapolations of pharmacokinetic parameters estimated from adult data. In some instances, the dose of the anti-TIGIT antagonist antibody (e.g., an anti-TIGIT antagonist antibody as disclosed herein, e.g., tiragolumab) is a tiered dose based on a subject's body weight (e.g., body weight (BW) >40 kg: 600 mg, BW >15 kg and ≤40 kg: 400 mg, and BW≤15 kg: 300 mg). In some instances, the dose of the PD-1 axis binding antagonist (e.g., anti-PD-L1 antagonist antibody (e.g., atezolizumab)) is a dose based on a subject's body weight (e.g., 15 mg/kg). In some instances, the dose of the PD-1 axis binding antagonist (e.g., anti-PD-L1 antagonist antibody (e.g., atezolizumab)) is a dose based on a subject's body surface area (e.g., body surface area (BSA) >1.25 m²: 600 mg, BSA >0.75 m² and ≤1.25 m²: 450 mg, BSA >0.5 m² and ≤0.75 m²: 350 mg, and BSA ≤0.5 m²: 300 mg). In some instances, the dose (e.g., about 600 mg) of the anti-TIGIT antagonist antibody (e.g., an anti-TIGIT antagonist antibody as disclosed herein, e.g., tiragolumab) is administered in combination with a dose of the PD-1 axis binding antagonist (e.g., anti-PD-L1 antagonist antibody (e.g., atezolizumab)) based on a subject's body weight (e.g., 15 mg/kg) every three weeks. In some instances, the tiered dose (e.g., body weight (BW) >40 kg: 600 mg, BW >15 kg and ≤40 kg: 400 mg, and BW≤15 kg: 300 mg) of the anti-TIGIT antagonist antibody (e.g., an anti-TIGIT antagonist antibody as disclosed herein, e.g., tiragolumab) is administered in combination with a dose of the PD-1 axis binding antagonist (e.g., anti-PD-L1 antagonist antibody (e.g., atezolizumab)) based on a subject's body weight (e.g., 15 mg/kg) every three weeks. In some instances, the tiered dose (e.g., body weight (BW) >40 kg: 600 mg, BW >15 kg and ≤40 kg: 400 mg, and BW≤15 kg: 300 mg) of the anti-TIGIT antagonist antibody (e.g., an anti-TIGIT antagonist antibody as disclosed herein, e.g., tiragolumab) is administered in combination with a dose of the PD-1 axis binding antagonist (e.g., anti-PD-L1 antagonist antibody (e.g., atezolizumab)) based on a subject's body surface area (e.g., BSA >1.25 m²: 600 mg, BSA >0.75 m² and ≤1.25 m²: 450 mg, BSA >0.5 m² and ≤0.75 m²: 350 mg, and BSA ≤0.5 m²: 300 mg) every three weeks. In some embodiments, the PD-1 axis binding antagonist (e.g., anti-PD-L1 antagonist antibody (e.g., atezolizumab)) is administered at a maximum dose of 1200 mg every three weeks. In some instances, the combination therapy is administered with one or more chemotherapeutic agents (e.g., a platinum-based chemotherapeutic agent (e.g., carboplatin or cisplatin) and/or a non-platinum-based chemotherapeutic agent (e.g., an antimetabolite (e.g., pemetrexed or gemcitabine)).

In some instances, the effective amount of the anti-TIGIT antagonist antibody (e.g., an anti-TIGIT antagonist antibody as disclosed herein, e.g., tiragolumab) to treat a subject having a cancer is a tiered dose based on a subject's body weight, wherein the subject has a body weight of (a) less than or equal to 15 kg, and the anti-TIGIT antagonist antibody is administered at a dose of between about 10 mg to about 1000 mg every three weeks (e.g., about 300 mg every three weeks); (b) greater than 15 kg and less than or equal to 40 kg, and the anti-TIGIT antagonist antibody is administered at a dose of between about 10 mg to about 1000 mg every three weeks (e.g., about 400 mg every three weeks); or (c) greater than 40 kg, and the anti-TIGIT antagonist antibody is administered at a dose of between about 30 mg to about 1200 mg every three weeks (e.g., about 600 mg every three weeks). In some instances, the effective amount of the anti-TIGIT antagonist antibody (e.g., an anti-TIGIT antagonist antibody as disclosed herein, e.g., tiragolumab) is a tiered dose based on a subject's body weight, wherein the subject has a body weight of (a) less than or equal to 15 kg, and the anti-TIGIT antagonist antibody is administered at a dose of between about 250 mg to about 350 mg every three weeks (e.g., about 300 mg every three weeks); (b) greater than 15 kg and less than or equal to 40 kg, and the anti-TIGIT antagonist antibody is administered at a dose of between about 350 mg to about 450 mg every three weeks (e.g., about 400 mg every three weeks); or (c) greater than 40 kg, and the anti-TIGIT antagonist antibody is administered at a dose of between about 550 mg to about 650 mg every three weeks (e.g., about 600 mg every three weeks). In some instances, the effective amount of the anti-TIGIT antagonist antibody (e.g., an anti-TIGIT antagonist antibody as disclosed herein, e.g., tiragolumab) is a tiered dose based on a subject's body weight, wherein the subject has a body weight of (a) less than or equal to 15 kg, and the anti-TIGIT antagonist antibody is administered at a dose of about 300 mg every three weeks; (b) greater than 15 kg and less than or equal to 40 kg, and the anti-TIGIT antagonist antibody is administered at a dose of about 400 mg every three weeks; or (c) greater than 40 kg, and the anti-TIGIT antagonist antibody is administered at a dose of about 600 mg every three weeks. In some instances, a dose of between about 0.01 mg/kg to about 50 mg/kg of the subject's body weight (e.g., between about 0.01 mg/kg to about 45 mg/kg, e.g., between about 0.1 mg/kg to about 40 mg/kg, e.g., between about 1 mg/kg to about 35 mg/kg, e.g., between about 2.5 mg/kg to about 30 mg/kg, e.g., between about 5 mg/kg to about 25 mg/kg, e.g., between about 10 mg/kg to about 20 mg/kg, e.g., between about 12.5 mg/kg to about 15 mg/kg, e.g., about 15±2 mg/kg, about 15±1 mg/kg, about 15±0.5 mg/kg, about 15±0.2 mg/kg, or about 15±0.1 mg/kg, e.g., about 15 mg/kg) of the PD-1 axis binding antagonist (e.g., anti-PD-L1 antagonist antibody (e.g., atezolizumab)) is administered in combination with a tiered dose based on a subject's body weight of the anti-TIGIT antagonist antibody (e.g., an anti-TIGIT antagonist antibody as disclosed herein, e.g., tiragolumab), wherein the subject has a body weight of (a) less than or equal to 15 kg, and the anti-TIGIT antagonist antibody is administered at a dose of between about 10 mg to about 1000 mg every three weeks (e.g., about 300 mg every three weeks); (b) greater than 15 kg and less than or equal to 40 kg, and the anti-TIGIT antagonist antibody is administered at a dose of between about 10 mg to about 1000 mg every three weeks (e.g., about 400 mg every three weeks); or (c) greater than 40 kg, and the anti-TIGIT antagonist antibody is administered at a dose of between about 30 mg to about 1200 mg every three weeks (e.g., about 600 mg every three weeks). In some instances, a subject with a body weight of less than or equal to 15 kg is administered a dose of between about 10 mg to about 1000 mg every three weeks (e.g., about 300 mg every three weeks) of the anti-TIGIT antagonist antibody (e.g., an anti-TIGIT antagonist antibody as disclosed herein, e.g., tiragolumab) and a dose of between about 0.01 mg/kg to about 50 mg/kg of the subject's body weight (e.g., between about 0.01 mg/kg to about 45 mg/kg, e.g., between about 0.1 mg/kg to about 40 mg/kg, e.g., between about 1 mg/kg to about 35 mg/kg, e.g., between about 2.5 mg/kg to about 30 mg/kg, e.g., between about 5 mg/kg to about 25 mg/kg, e.g., between about 10 mg/kg to about 20 mg/kg, e.g., between about 12.5 mg/kg to about 15 mg/kg, e.g., about 15±2 mg/kg, about 15±1 mg/kg, about 15±0.5 mg/kg, about 15±0.2 mg/kg, or about 15±0.1 mg/kg, e.g., about 15 mg/kg) of the PD-1 axis binding antagonist (e.g., anti-PD-L1 antagonist antibody (e.g., atezolizumab)) every three weeks. In some instances, a subject with a body weight of greater than 15 kg and less than or equal to 40 kg is administered a dose of between about 10 mg to about 1000 mg every three weeks (e.g., about 400 mg every three weeks) of the anti-TIGIT antagonist antibody (e.g., an anti-TIGIT antagonist antibody as disclosed herein, e.g., tiragolumab) and a dose of between about 0.01 mg/kg to about 50 mg/kg of the subject's body weight (e.g., between about 0.01 mg/kg to about 45 mg/kg, e.g., between about 0.1 mg/kg to about 40 mg/kg, e.g., between about 1 mg/kg to about 35 mg/kg, e.g., between about 2.5 mg/kg to about 30 mg/kg, e.g., between about 5 mg/kg to about 25 mg/kg, e.g., between about 10 mg/kg to about 20 mg/kg, e.g., between about 12.5 mg/kg to about 15 mg/kg, e.g., about 15±2 mg/kg, about 15±1 mg/kg, about 15±0.5 mg/kg, about 15±0.2 mg/kg, or about 15±0.1 mg/kg, e.g., about 15 mg/kg) of the PD-1 axis binding antagonist (e.g., anti-PD-L1 antagonist antibody (e.g., atezolizumab)) every three weeks. In some instances, a subject with a body weight of greater than 40 kg is administered a dose of between about 30 mg to about 1200 mg every three weeks (e.g., about 600 mg every three weeks) of the anti-TIGIT antagonist antibody (e.g., an anti-TIGIT antagonist antibody as disclosed herein, e.g., tiragolumab) and a dose of between about 0.01 mg/kg to about 50 mg/kg of the subject's body weight (e.g., between about 0.01 mg/kg to about 45 mg/kg, e.g., between about 0.1 mg/kg to about 40 mg/kg, e.g., between about 1 mg/kg to about 35 mg/kg, e.g., between about 2.5 mg/kg to about 30 mg/kg, e.g., between about 5 mg/kg to about 25 mg/kg, e.g., between about 10 mg/kg to about 20 mg/kg, e.g., between about 12.5 mg/kg to about 15 mg/kg, e.g., about 15±2 mg/kg, about 15±1 mg/kg, about 15±0.5 mg/kg, about 15±0.2 mg/kg, or about 15±0.1 mg/kg, e.g., about 15 mg/kg) of the PD-1 axis binding antagonist (e.g., anti-PD-L1 antagonist antibody (e.g., atezolizumab)) every three weeks.

In some instances, the effective amount of the anti-TIGIT antagonist antibody (e.g., an anti-TIGIT antagonist antibody as disclosed herein, e.g., tiragolumab) to treat a subject having a cancer is a tiered dose based on a subject's body weight, wherein the subject has a body weight of (a) less than or equal to 15 kg, and the anti-TIGIT antagonist antibody is administered at a dose of between 10 mg to 1000 mg every three weeks (e.g., 300 mg every three weeks); (b) greater than 15 kg and less than or equal to 40 kg, and the anti-TIGIT antagonist antibody is administered at a dose of between 10 mg to 1000 mg every three weeks (e.g., 400 mg every three weeks); or (c) greater than 40 kg, and the anti-TIGIT antagonist antibody is administered at a dose of between 30 mg to 1200 mg every three weeks (e.g., 600 mg every three weeks). In some instances, the effective amount of the anti-TIGIT antagonist antibody (e.g., an anti-TIGIT antagonist antibody as disclosed herein, e.g., tiragolumab) is a tiered dose based on a subject's body weight, wherein the subject has a body weight of (a) less than or equal to 15 kg, and the anti-TIGIT antagonist antibody is administered at a dose of between 250 mg to 350 mg every three weeks (e.g., 300 mg every three weeks); (b) greater than 15 kg and less than or equal to 40 kg, and the anti-TIGIT antagonist antibody is administered at a dose of between 350 mg to 450 mg every three weeks (e.g., 400 mg every three weeks); or (c) greater than 40 kg, and the anti-TIGIT antagonist antibody is administered at a dose of between 550 mg to 650 mg every three weeks (e.g., 600 mg every three weeks). In some instances, the effective amount of the anti-TIGIT antagonist antibody (e.g., an anti-TIGIT antagonist antibody as disclosed herein, e.g., tiragolumab) is a tiered dose based on a subject's body weight, wherein the subject has a body weight of (a) less than or equal to 15 kg, and the anti-TIGIT antagonist antibody is administered at a dose of 300 mg every three weeks; (b) greater than 15 kg and less than or equal to 40 kg, and the anti-TIGIT antagonist antibody is administered at a dose of 400 mg every three weeks; or (c) greater than 40 kg, and the anti-TIGIT antagonist antibody is administered at a dose of 600 mg every three weeks. In some instances, a dose of between 0.01 mg/kg to 50 mg/kg of the subject's body weight (e.g., between 0.01 mg/kg to 45 mg/kg, e.g., between 0.1 mg/kg to 40 mg/kg, e.g., between 1 mg/kg to 35 mg/kg, e.g., between 2.5 mg/kg to 30 mg/kg, e.g., between 5 mg/kg to 25 mg/kg, e.g., between 10 mg/kg to 20 mg/kg, e.g., between 12.5 mg/kg to 15 mg/kg, e.g., 15±2 mg/kg, 15±1 mg/kg, 15±0.5 mg/kg, 15±0.2 mg/kg, or 15±0.1 mg/kg, e.g., 15 mg/kg) of the PD-1 axis binding antagonist (e.g., anti-PD-L1 antagonist antibody (e.g., atezolizumab)) is administered in combination with a tiered dose based on a subject's body weight of the anti-TIGIT antagonist antibody (e.g., an anti-TIGIT antagonist antibody as disclosed herein, e.g., tiragolumab), wherein the subject has a body weight of (a) less than or equal to 15 kg, and the anti-TIGIT antagonist antibody is administered at a dose of between 10 mg to 1000 mg every three weeks (e.g., 300 mg every three weeks); (b) greater than 15 kg and less than or equal to 40 kg, and the anti-TIGIT antagonist antibody is administered at a dose of between 10 mg to 1000 mg every three weeks (e.g., 400 mg every three weeks); or (c) greater than 40 kg, and the anti-TIGIT antagonist antibody is administered at a dose of between 30 mg to 1200 mg every three weeks (e.g., 600 mg every three weeks). In some instances, a subject with a body weight of less than or equal to 15 kg is administered a dose of between 10 mg to 1000 mg every three weeks (e.g., 300 mg every three weeks) of the anti-TIGIT antagonist antibody (e.g., an anti-TIGIT antagonist antibody as disclosed herein, e.g., tiragolumab) and a dose of between 0.01 mg/kg to 50 mg/kg of the subject's body weight (e.g., between 0.01 mg/kg to 45 mg/kg, e.g., between 0.1 mg/kg to 40 mg/kg, e.g., between 1 mg/kg to 35 mg/kg, e.g., between 2.5 mg/kg to 30 mg/kg, e.g., between 5 mg/kg to 25 mg/kg, e.g., between 10 mg/kg to 20 mg/kg, e.g., between 12.5 mg/kg to 15 mg/kg, e.g., 15±2 mg/kg, 15±1 mg/kg, 15±0.5 mg/kg, 15±0.2 mg/kg, or 15±0.1 mg/kg, e.g., 15 mg/kg) of the PD-1 axis binding antagonist (e.g., anti-PD-L1 antagonist antibody (e.g., atezolizumab)) every three weeks. In some instances, a subject with a body weight of greater than 15 kg and less than or equal to 40 kg is administered a dose of between 10 mg to 1000 mg every three weeks (e.g., 400 mg every three weeks) of the anti-TIGIT antagonist antibody (e.g., an anti-TIGIT antagonist antibody as disclosed herein, e.g., tiragolumab) and a dose of between 0.01 mg/kg to 50 mg/kg of the subject's body weight (e.g., between 0.01 mg/kg to 45 mg/kg, e.g., between 0.1 mg/kg to 40 mg/kg, e.g., between 1 mg/kg to 35 mg/kg, e.g., between 2.5 mg/kg to 30 mg/kg, e.g., between 5 mg/kg to 25 mg/kg, e.g., between 10 mg/kg to 20 mg/kg, e.g., between 12.5 mg/kg to 15 mg/kg, e.g., 15±2 mg/kg, 15±1 mg/kg, 15±0.5 mg/kg, 15±0.2 mg/kg, or 15±0.1 mg/kg, e.g., 15 mg/kg) of the PD-1 axis binding antagonist (e.g., anti-PD-L1 antagonist antibody (e.g., atezolizumab)) every three weeks. In some instances, a subject with a body weight of greater than 40 kg is administered a dose of between 30 mg to 1200 mg every three weeks (e.g., 600 mg every three weeks) of the anti-TIGIT antagonist antibody (e.g., an anti-TIGIT antagonist antibody as disclosed herein, e.g., tiragolumab) and a dose of between 0.01 mg/kg to 50 mg/kg of the subject's body weight (e.g., between 0.01 mg/kg to 45 mg/kg, e.g., between 0.1 mg/kg to 40 mg/kg, e.g., between 1 mg/kg to 35 mg/kg, e.g., between 2.5 mg/kg to 30 mg/kg, e.g., between 5 mg/kg to 25 mg/kg, e.g., between 10 mg/kg to 20 mg/kg, e.g., between 12.5 mg/kg to 15 mg/kg, e.g., 15±2 mg/kg, 15±1 mg/kg, 15±0.5 mg/kg, 15±0.2 mg/kg, or 15±0.1 mg/kg, e.g., 15 mg/kg) of the PD-1 axis binding antagonist (e.g., anti-PD-L1 antagonist antibody (e.g., atezolizumab)) every three weeks.

In some instances, the effective amount of the anti-TIGIT antagonist antibody (e.g., an anti-TIGIT antagonist antibody as disclosed herein, e.g., tiragolumab) to treat a subject having a cancer is a tiered dose based on a subject's body surface area. In some instances, the effective amount of the anti-TIGIT antagonist antibody (e.g., an anti-TIGIT antagonist antibody as disclosed herein, e.g., tiragolumab) is a tiered dose based on a subject's body surface area, wherein the subject has a body surface area of (a) less than or equal to 0.5 m², and the anti-TIGIT antagonist antibody is administered at a dose of between about 10 mg to about 1000 mg every three weeks (e.g., about 300 mg every three weeks); (b) greater than 0.5 m² and less than or equal to 0.75 m², and the anti-TIGIT antagonist antibody is administered at a dose of between about 10 mg to about 1000 mg every three weeks (e.g., about 350 mg every three weeks); (c) greater than 0.75 m² and less than or equal to 1.25 m², and the anti-TIGIT antagonist antibody is administered at a dose of between about 10 mg to about 1000 mg every three weeks (e.g., about 450 mg every three weeks); or (d) greater than 1.25 m², and the anti-TIGIT antagonist antibody is administered at a dose of between about 30 mg to about 1200 mg every three weeks (e.g., about 600 mg every three weeks). In some instances, the effective amount of the anti-TIGIT antagonist antibody (e.g., an anti-TIGIT antagonist antibody as disclosed herein, e.g., tiragolumab) is a tiered dose based on a subject's body surface area, wherein the subject has a body surface area of (a) less than or equal to 0.5 m², and the anti-TIGIT antagonist antibody is administered at a dose of between about 250 mg to about 350 mg every three weeks (e.g., about 300 mg every three weeks); (b) greater than 0.5 m² and less than or equal to 0.75 m², and the anti-TIGIT antagonist antibody is administered at a dose of between about 300 mg to about 400 mg every three weeks (e.g., about 350 mg every three weeks); or (c) greater than 0.75 m² and less than or equal to 1.25 m², and the anti-TIGIT antagonist antibody is administered at a dose of between about 400 mg to about 500 mg every three weeks (e.g., about 450 mg every three weeks); or (d) greater than 1.25 m², and the anti-TIGIT antagonist antibody is administered at a dose of between about 550 mg to about 650 mg every three weeks (e.g., about 600 mg every three weeks). In some instances, the effective amount of the anti-TIGIT antagonist antibody (e.g., an anti-TIGIT antagonist antibody as disclosed herein, e.g., tiragolumab) is a tiered dose based on a subject's body surface area, wherein the subject has a body surface area of (a) less than or equal to 0.5 m², and the anti-TIGIT antagonist antibody is administered at a dose of about 300 mg every three weeks; (b) greater than 0.5 m² and less than or equal to 0.75 m², and the anti-TIGIT antagonist antibody is administered at a dose of about 400 mg every three weeks; (c) greater than 0.75 m² and less than or equal to 1.25 m², and the anti-TIGIT antagonist antibody is administered at a dose of 450 mg every three weeks; or (d) greater than 1.25 m², and the anti-TIGIT antagonist antibody is administered at a dose of about 600 mg every three weeks.

In some instances, a dose of between about 0.01 mg/kg to about 50 mg/kg of the subject's body weight (e.g., between about 0.01 mg/kg to about 45 mg/kg, e.g., between about 0.1 mg/kg to about 40 mg/kg, e.g., between about 1 mg/kg to about 35 mg/kg, e.g., between about 2.5 mg/kg to about 30 mg/kg, e.g., between about 5 mg/kg to about 25 mg/kg, e.g., between about 10 mg/kg to about 20 mg/kg, e.g., between about 12.5 mg/kg to about 15 mg/kg, e.g., about 15±2 mg/kg, about 15±1 mg/kg, about 15±0.5 mg/kg, about 15±0.2 mg/kg, or about 15±0.1 mg/kg, e.g., about 15 mg/kg) of the PD-1 axis binding antagonist (e.g., anti-PD-L1 antagonist antibody (e.g., atezolizumab)) is administered in combination with a tiered dose based on a subject's body surface area of the anti-TIGIT antagonist antibody (e.g., an anti-TIGIT antagonist antibody as disclosed herein, e.g., tiragolumab), wherein the subject has a body surface area of (a) less than or equal to 0.5 m², and the anti-TIGIT antagonist antibody is administered at a dose of between about 10 mg to about 1000 mg every three weeks (e.g., about 300 mg every three weeks); (b) greater than 0.5 m² and less than or equal to 0.75 m², and the anti-TIGIT antagonist antibody is administered at a dose of between about 10 mg to about 1000 mg every three weeks (e.g., about 350 mg every three weeks); (c) greater than 0.75 m² and less than or equal to 1.25 m², and the anti-TIGIT antagonist antibody is administered at a dose of between about 10 mg to about 1000 mg every three weeks (e.g., about 450 mg every three weeks); or (d) greater than 1.25 m², and the anti-TIGIT antagonist antibody is administered at a dose of between about 30 mg to about 1200 mg every three weeks (e.g., about 600 mg every three weeks). In some instances, a subject with a body surface area of less than or equal to 0.5 m² is administered a dose of between about 10 mg to about 1000 mg every three weeks (e.g., about 300 mg every three weeks) of the anti-TIGIT antagonist antibody (e.g., an anti-TIGIT antagonist antibody as disclosed herein, e.g., tiragolumab) and a dose of between about 0.01 mg/kg to about 50 mg/kg of the subject's body weight (e.g., between about 0.01 mg/kg to about 45 mg/kg, e.g., between about 0.1 mg/kg to about 40 mg/kg, e.g., between about 1 mg/kg to about 35 mg/kg, e.g., between about 2.5 mg/kg to about 30 mg/kg, e.g., between about 5 mg/kg to about 25 mg/kg, e.g., between about 10 mg/kg to about 20 mg/kg, e.g., between about 12.5 mg/kg to about 15 mg/kg, e.g., about 15±2 mg/kg, about 15±1 mg/kg, about 15±0.5 mg/kg, about 15±0.2 mg/kg, or about 15±0.1 mg/kg, e.g., about 15 mg/kg) of the PD-1 axis binding antagonist (e.g., anti-PD-L1 antagonist antibody (e.g., atezolizumab)) every three weeks. In some instances, a subject with a body surface area of greater than 0.5 m² and less than or equal to 0.75 m² is administered a dose of between about 10 mg to about 1000 mg every three weeks (e.g., about 350 mg every three weeks) of the anti-TIGIT antagonist antibody (e.g., an anti-TIGIT antagonist antibody as disclosed herein, e.g., tiragolumab) and a dose of between about 0.01 mg/kg to about 50 mg/kg of the subject's body weight (e.g., between about 0.01 mg/kg to about 45 mg/kg, e.g., between about 0.1 mg/kg to about 40 mg/kg, e.g., between about 1 mg/kg to about 35 mg/kg, e.g., between about 2.5 mg/kg to about 30 mg/kg, e.g., between about 5 mg/kg to about 25 mg/kg, e.g., between about 10 mg/kg to about 20 mg/kg, e.g., between about 12.5 mg/kg to about 15 mg/kg, e.g., about 15±2 mg/kg, about 15±1 mg/kg, about 15±0.5 mg/kg, about 15±0.2 mg/kg, or about 15±0.1 mg/kg, e.g., about 15 mg/kg) of the PD-1 axis binding antagonist (e.g., anti-PD-L1 antagonist antibody (e.g., atezolizumab)) every three weeks. In some instances, a subject with a body surface area of greater than 0.75 m² and less than or equal to 1.25 m² is administered a dose of between about 10 mg to about 1000 mg every three weeks (e.g., about 450 mg every three weeks) of the anti-TIGIT antagonist antibody (e.g., an anti-TIGIT antagonist antibody as disclosed herein, e.g., tiragolumab) and a dose of between about 0.01 mg/kg to about 50 mg/kg of the subject's body weight (e.g., between about 0.01 mg/kg to about 45 mg/kg, e.g., between about 0.1 mg/kg to about 40 mg/kg, e.g., between about 1 mg/kg to about 35 mg/kg, e.g., between about 2.5 mg/kg to about 30 mg/kg, e.g., between about 5 mg/kg to about 25 mg/kg, e.g., between about 10 mg/kg to about 20 mg/kg, e.g., between about 12.5 mg/kg to about 15 mg/kg, e.g., about 15±2 mg/kg, about 15±1 mg/kg, about 15±0.5 mg/kg, about 15+0.2 mg/kg, or about 15±0.1 mg/kg, e.g., about 15 mg/kg) of the PD-1 axis binding antagonist (e.g., anti-PD-L1 antagonist antibody (e.g., atezolizumab)) every three weeks. In some instances, a subject with a body surface area of greater than 1.25 m² is administered a dose of between about 30 mg to about 1200 mg every three weeks (e.g., about 600 mg every three weeks) of the anti-TIGIT antagonist antibody (e.g., an anti-TIGIT antagonist antibody as disclosed herein, e.g., tiragolumab) and a dose of between about 0.01 mg/kg to about 50 mg/kg of the subject's body weight (e.g., between about 0.01 mg/kg to about 45 mg/kg, e.g., between about 0.1 mg/kg to about 40 mg/kg, e.g., between about 1 mg/kg to about 35 mg/kg, e.g., between about 2.5 mg/kg to about 30 mg/kg, e.g., between about 5 mg/kg to about 25 mg/kg, e.g., between about 10 mg/kg to about 20 mg/kg, e.g., between about 12.5 mg/kg to about 15 mg/kg, e.g., about 15±2 mg/kg, about 15±1 mg/kg, about 15±0.5 mg/kg, about 15±0.2 mg/kg, or about 15±0.1 mg/kg, e.g., about 15 mg/kg) of the PD-1 axis binding antagonist (e.g., anti-PD-L1 antagonist antibody (e.g., atezolizumab)) every three weeks.

In some instances, the effective amount of the anti-TIGIT antagonist antibody (e.g., an anti-TIGIT antagonist antibody as disclosed herein, e.g., tiragolumab) is a tiered dose based on a subject's body surface area, wherein the subject has a body surface area of (a) less than or equal to 0.5 m², and the anti-TIGIT antagonist antibody is administered at a dose of between 10 mg to 1000 mg every three weeks (e.g., 300 mg every three weeks); (b) greater than 0.5 m² and less than or equal to 0.75 m², and the anti-TIGIT antagonist antibody is administered at a dose of between 10 mg to 1000 mg every three weeks (e.g., 350 mg every three weeks); (c) greater than 0.75 m² and less than or equal to 1.25 m², and the anti-TIGIT antagonist antibody is administered at a dose of between 10 mg to 1000 mg every three weeks (e.g., 450 mg every three weeks); or (d) greater than 1.25 m², and the anti-TIGIT antagonist antibody is administered at a dose of between 30 mg to 1200 mg every three weeks (e.g., 600 mg every three weeks). In some instances, the effective amount of the anti-TIGIT antagonist antibody (e.g., an anti-TIGIT antagonist antibody as disclosed herein, e.g., tiragolumab) is a tiered dose based on a subject's body surface area, wherein the subject has a body surface area of (a) less than or equal to 0.5 m², and the anti-TIGIT antagonist antibody is administered at a dose of between 250 mg to 350 mg every three weeks (e.g., 300 mg every three weeks); (b) greater than 0.5 m² and less than or equal to 0.75 m², and the anti-TIGIT antagonist antibody is administered at a dose of between 300 mg to 400 mg every three weeks (e.g., 350 mg every three weeks); or (c) greater than 0.75 m² and less than or equal to 1.25 m², and the anti-TIGIT antagonist antibody is administered at a dose of between 400 mg to 500 mg every three weeks (e.g., 450 mg every three weeks); or (d) greater than 1.25 m², and the anti-TIGIT antagonist antibody is administered at a dose of between 550 mg to 650 mg every three weeks (e.g., 600 mg every three weeks). In some instances, the effective amount of the anti-TIGIT antagonist antibody (e.g., an anti-TIGIT antagonist antibody as disclosed herein, e.g., tiragolumab) is a tiered dose based on a subject's body surface area, wherein the subject has a body surface area of (a) less than or equal to 0.5 m², and the anti-TIGIT antagonist antibody is administered at a dose of 300 mg every three weeks; (b) greater than 0.5 m² and less than or equal to 0.75 m², and the anti-TIGIT antagonist antibody is administered at a dose of 400 mg every three weeks; (c) greater than 0.75 m² and less than or equal to 1.25 m², and the anti-TIGIT antagonist antibody is administered at a dose of 450 mg every three weeks; or (d) greater than 1.25 m², and the anti-TIGIT antagonist antibody is administered at a dose of 600 mg every three weeks.

In some instances, a dose of between 0.01 mg/kg to 50 mg/kg of the subject's body weight (e.g., between 0.01 mg/kg to 45 mg/kg, e.g., between 0.1 mg/kg to 40 mg/kg, e.g., between 1 mg/kg to 35 mg/kg, e.g., between 2.5 mg/kg to 30 mg/kg, e.g., between 5 mg/kg to 25 mg/kg, e.g., between 10 mg/kg to 20 mg/kg, e.g., between 12.5 mg/kg to 15 mg/kg, e.g., 15±2 mg/kg, 15±1 mg/kg, 15±0.5 mg/kg, 15±0.2 mg/kg, or 15±0.1 mg/kg, e.g., 15 mg/kg) of the PD-1 axis binding antagonist (e.g., anti-PD-L1 antagonist antibody (e.g., atezolizumab)) is administered in combination with a tiered dose based on a subject's body surface area of the anti-TIGIT antagonist antibody (e.g., an anti-TIGIT antagonist antibody as disclosed herein, e.g., tiragolumab), wherein the subject has a body surface area of (a) less than or equal to 0.5 m², and the anti-TIGIT antagonist antibody is administered at a dose of between 10 mg to 1000 mg every three weeks (e.g., 300 mg every three weeks); (b) greater than 0.5 m² and less than or equal to 0.75 m², and the anti-TIGIT antagonist antibody is administered at a dose of between 10 mg to 1000 mg every three weeks (e.g., 350 mg every three weeks); (c) greater than 0.75 m² and less than or equal to 1.25 m², and the anti-TIGIT antagonist antibody is administered at a dose of between 10 mg to 1000 mg every three weeks (e.g., 450 mg every three weeks); or (d) greater than 1.25 m², and the anti-TIGIT antagonist antibody is administered at a dose of between 30 mg to 1200 mg every three weeks (e.g., 600 mg every three weeks). In some instances, a subject with a body surface area of less than or equal to 0.5 m² is administered a dose of between 10 mg to 1000 mg every three weeks (e.g., 300 mg every three weeks) of the anti-TIGIT antagonist antibody (e.g., an anti-TIGIT antagonist antibody as disclosed herein, e.g., tiragolumab) and a dose of between 0.01 mg/kg to 50 mg/kg of the subject's body weight (e.g., between 0.01 mg/kg to 45 mg/kg, e.g., between 0.1 mg/kg to 40 mg/kg, e.g., between 1 mg/kg to 35 mg/kg, e.g., between 2.5 mg/kg to 30 mg/kg, e.g., between 5 mg/kg to 25 mg/kg, e.g., between 10 mg/kg to 20 mg/kg, e.g., between 12.5 mg/kg to 15 mg/kg, e.g., 15±2 mg/kg, 15±1 mg/kg, 15±0.5 mg/kg, 15±0.2 mg/kg, or 15±0.1 mg/kg, e.g., 15 mg/kg) of the PD-1 axis binding antagonist (e.g., anti-PD-L1 antagonist antibody (e.g., atezolizumab)) every three weeks. In some instances, a subject with a body surface area of greater than 0.5 m² and less than or equal to 0.75 m² is administered a dose of between 10 mg to 1000 mg every three weeks (e.g., 350 mg every three weeks) of the anti-TIGIT antagonist antibody (e.g., an anti-TIGIT antagonist antibody as disclosed herein, e.g., tiragolumab) and a dose of between 0.01 mg/kg to 50 mg/kg of the subject's body weight (e.g., between 0.01 mg/kg to 45 mg/kg, e.g., between 0.1 mg/kg to 40 mg/kg, e.g., between 1 mg/kg to 35 mg/kg, e.g., between 2.5 mg/kg to 30 mg/kg, e.g., between 5 mg/kg to 25 mg/kg, e.g., between 10 mg/kg to 20 mg/kg, e.g., between 12.5 mg/kg to 15 mg/kg, e.g., 15±2 mg/kg, 15±1 mg/kg, 15±0.5 mg/kg, 15±0.2 mg/kg, or 15±0.1 mg/kg, e.g., 15 mg/kg) of the PD-1 axis binding antagonist (e.g., anti-PD-L1 antagonist antibody (e.g., atezolizumab)) every three weeks. In some instances, a subject with a body surface area of greater than 0.75 m² and less than or equal to 1.25 m² is administered a dose of between 10 mg to 1000 mg every three weeks (e.g., 450 mg every three weeks) of the anti-TIGIT antagonist antibody (e.g., an anti-TIGIT antagonist antibody as disclosed herein, e.g., tiragolumab) and a dose of between 0.01 mg/kg to 50 mg/kg of the subject's body weight (e.g., between 0.01 mg/kg to 45 mg/kg, e.g., between 0.1 mg/kg to 40 mg/kg, e.g., between 1 mg/kg to 35 mg/kg, e.g., between 2.5 mg/kg to 30 mg/kg, e.g., between 5 mg/kg to 25 mg/kg, e.g., between 10 mg/kg to 20 mg/kg, e.g., between 12.5 mg/kg to 15 mg/kg, e.g., 15+2 mg/kg, 15±1 mg/kg, 15±0.5 mg/kg, 15 0.2 mg/kg, or 15±0.1 mg/kg, e.g., 15 mg/kg) of the PD-1 axis binding antagonist (e.g., anti-PD-L1 antagonist antibody (e.g., atezolizumab)) every three weeks. In some instances, a subject with a body surface area of greater than 1.25 m² is administered a dose of between 30 mg to 1200 mg every three weeks (e.g., 600 mg every three weeks) of the anti-TIGIT antagonist antibody (e.g., an anti-TIGIT antagonist antibody as disclosed herein, e.g., tiragolumab) and a dose of between 0.01 mg/kg to 50 mg/kg of the subject's body weight (e.g., between 0.01 mg/kg to 45 mg/kg, e.g., between 0.1 mg/kg to 40 mg/kg, e.g., between 1 mg/kg to 35 mg/kg, e.g., between 2.5 mg/kg to 30 mg/kg, e.g., between 5 mg/kg to 25 mg/kg, e.g., between 10 mg/kg to 20 mg/kg, e.g., between 12.5 mg/kg to 15 mg/kg, e.g., 15±2 mg/kg, 15±1 mg/kg, 15±0.5 mg/kg, 15±0.2 mg/kg, or 15±0.1 mg/kg, e.g., 15 mg/kg) of the PD-1 axis binding antagonist (e.g., anti-PD-L1 antagonist antibody (e.g., atezolizumab)) every three weeks.

Dosing of Chemotherapeutic Agents

Therapeutically effective amounts of various chemotherapeutic agents are known in the art and contemplated in the present invention. In particular instances, one or more chemotherapeutic agents (e.g., a platinum-based chemotherapeutic agent (e.g., carboplatin or cisplatin) and/or one or more non-platinum-based chemotherapeutic agents (e.g., an alkylating agent (e.g., cyclophosphamide), a taxane (e.g., paclitaxel, e.g., nab-paclitaxel), and/or a topoisomerase II inhibitor (e.g., doxorubicin)) are administered according to the doses recited herein.

### Platinum-Based Chemotherapeutic Agents

In some instances, the effective amount of a platinum-based chemotherapeutic agent (e.g., carboplatin or cisplatin) is a dose sufficient to achieve an AUC from 1-50 mg/ml/min (e.g., 2-25 mg/ml/min, 3-15 mg/ml/min, 4-10 mg/ml/min, or 5 mg/ml/min, e.g., 2 mg/ml/min, 3 mg/ml/min, 4 mg/ml/min, 5 mg/ml/min, 6 mg/ml/min, 7 mg/ml/min, 8 mg/ml/min, 9 mg/ml/min, 10 mg/ml/min, 11 mg/ml/min, 12 mg/ml/min, 13 mg/ml/min, 14 mg/ml/min, 15 mg/ml/min, 20 mg/ml/min, 25 mg/ml/min, 30 mg/ml/min, 35 mg/ml/min, 40 mg/ml/min, 45 mg/ml/min, 50 mg/ml/min). In some instances, the effective amount of the platinum-based chemotherapeutic agent (e.g., carboplatin or cisplatin) is a dose sufficient to achieve an AUC=5 mg/ml/min. In some instances, the effective amount of the platinum-based chemotherapeutic agent (e.g., carboplatin or cisplatin) is a dose sufficient to achieve an AUC=6 mg/ml/min. In some instances, the effective amount of carboplatin is a dose sufficient to achieve an AUC=5 mg/ml/min. In some instances, the effective amount of carboplatin is a dose sufficient to achieve an AUC=6 mg/ml/min. In some instances, the effective amount of carboplatin is a dose sufficient to achieve an AUC=5 mg/ml/min on Day 1 of a 21-day dosing cycle. In some instances, the effective amount of carboplatin is a dose sufficient to achieve an AUC=6 mg/ml/min on Day 1 of a 21-day dosing cycle. In some instances, the effective amount of carboplatin is a dose sufficient to achieve an AUC=5 mg/ml/min when given after pemetrexed or gemcitabine. In some instances, the effective amount of carboplatin is a dose sufficient to achieve an AUC=6 mg/ml/min when given after paclitaxel.

AUC can be calculated using the Calvert formula (Calvert et al., J. Clin. Oncol. 1989, 7:1748-56):

Total dose (mg)=(target AUC)×(glomerular filtration rate [GFR]+25)

In some instances, the effective amount of the platinum-based chemotherapeutic agent (e.g., carboplatin or cisplatin) is 200 mg-1500 mg (e.g., 300 mg-1200 mg, 400 mg-1100 mg, or 500 mg-1000 mg, e.g., 300 mg-400 mg, 400 mg-500 mg, 500 mg-600 mg, 600 mg-700 mg, 700 mg-750 mg, 750 mg-800 mg, 800 mg-900 mg, 900 mg-1000 mg, 1000 mg-1100 mg, or 1100 mg-1200 mg, e.g., about 200 mg, about 300 mg, about 400 mg, about 500 mg, about 600 mg, about 700 mg, about 800 mg, about 900 mg, about 1000 mg, about 1100 mg, about 1200 mg, about 1300 mg, about 1400 mg, or about 1500 mg). In some instances, the effective amount of the platinum-based chemotherapeutic agent (e.g., carboplatin or cisplatin) is about 500 mg-1000 mg (e.g., about 500 mg, about 600 mg, about 700 mg, about 800 mg, about 900 mg, or about 1000 mg).

In some instances, the effective amount of the platinum-based chemotherapeutic agent (e.g., carboplatin or cisplatin) is between about 20 mg/m² to about 200 mg/m² (e.g., between about 20 mg/m² to about 150 mg/m², e.g., between about 30 mg/m² to about 125 mg/m², e.g., between about 40 mg/m² to about 110 mg/m², e.g., between about 50 mg/m² to about 100 mg/m², e.g., between about 60 mg/m² to about 90 mg/m², e.g., between about 70 mg/m² to about 80 mg/m², e.g., about 75 mg/m², e.g., 75 mg/m²). In some instances, the effective amount of the platinum-based chemotherapeutic agent (e.g., carboplatin or cisplatin) is about 75 mg/m². In some instances, the effective amount of cisplatin is about 75 mg/m². In some instances, the effective amount of cisplatin is about 75 mg/m² every three weeks. In some instances, the effective amount of the platinum-based chemotherapeutic agent (e.g., carboplatin or cisplatin) is between 20 mg/m² to 200 mg/m² (e.g., between 20 mg/m² to 150 mg/m², e.g., between 30 mg/m² to 125 mg/m², e.g., between 40 mg/m² to 110 mg/m², e.g., between 50 mg/m² to 100 mg/m², e.g., between 60 mg/m² to 90 mg/m², e.g., between 70 mg/m² to 80 mg/m², e.g., 75 mg/m², e.g., 75 mg/m²). In some instances, the effective amount of the platinum-based chemotherapeutic agent (e.g., carboplatin or cisplatin) is 75 mg/m². In some instances, the effective amount of cisplatin is 75 mg/m². In some instances, the effective amount of cisplatin is 75 mg/m² every three weeks.

In some instances, the platinum-based chemotherapeutic agent (e.g., carboplatin or cisplatin) is administered to the subject or population of subjects intravenously (e.g., over a 30-120-minute infusion). In some instances, carboplatin is administered intravenously over a 30-60-minute infusion. In some instances, cisplatin is administered intravenously over a 60-120-minute infusion. In some instances, the platinum-based chemotherapeutic agent (e.g., carboplatin or cisplatin) is administered to the subject or population of subjects every three weeks. In some instances, the platinum-based chemotherapeutic agent (e.g., carboplatin or cisplatin) is administered to the subject or population of subjects on about Day 1 (e.g., Day −3, Day −2, Day −1, Day 1, Day 2, or Day 3) of a 21-day dosing cycle.

Taxanes

A therapeutically effective amount of a taxane (e.g., paclitaxel or nab-paclitaxel (ABRAXANE®)) administered to a human will be in the range of about 25 to about 300 mg/m² (e.g., about 25 mg/m², about 50 mg/m², about 75 mg/m², about 80 mg/m², about 90 mg/m², about 100 mg/m², about 110 mg/m², about 120 mg/m², about 125 mg/m², about 130 mg/m², about 140 mg/m², about 150 mg/m², about 160 mg/m², about 170 mg/m², about 175 mg/m², about 180 mg/m², about 190 mg/m², about 200 mg/m², about 225 mg/m², about 250 mg/m², about 275 mg/m², or about 300 mg/m² (e.g., 25 mg/m², 50 mg/m², 75 mg/m², 80 mg/m², 90 mg/m², 100 mg/m², 110 mg/m², 120 mg/m², 125 mg/m², 130 mg/m², 140 mg/m², 150 mg/m², 160 mg/m², 170 mg/m², 175 mg/m², 180 mg/m², 190 mg/m², 200 mg/m², 225 mg/m², 250 mg/m², 275 mg/m², or 300 mg/m²)) whether by one or more administrations. For example, in some embodiments, about 175 mg/m² of paclitaxel is administered. In some embodiments, 175 mg/m² of paclitaxel is administered. In some embodiments, paclitaxel is administered at a dose of between about 175 mg/m² to about 200 mg/m² every three weeks. In some embodiments, paclitaxel is administered at a dose of about 175 mg/m² every three weeks. In some embodiments, paclitaxel is administered at a dose of about 200 mg/m² every three weeks. In some embodiments, paclitaxel is administered at a dose of between 175 mg/m² to 200 mg/m² every three weeks. In some embodiments, paclitaxel is administered at a dose of 175 mg/m² every three weeks. In some embodiments, paclitaxel is administered at a dose of 200 mg/m² every three weeks. In some embodiments, paclitaxel is administered at 175 mg/m² IV every 3 weeks. In some embodiments, paclitaxel is administered to a subject of Asian race/ethnicity at 175 mg/m² IV every 3 weeks. In some embodiments, paclitaxel is administered at 200 mg/m² IV every 3 weeks. In some embodiments, paclitaxel is administered to a subject of non-Asian race/ethnicity at 200 mg/m² IV every 3 weeks. In some embodiments, about 100 mg/m² of nab-paclitaxel (ABRAXANE®) is administered. In some embodiments, 100 mg/m² of nab-paclitaxel (ABRAXANE®) is administered. In some embodiments, nab-paclitaxel (ABRAXANE®) is administered at 100 mg/m² IV every week. In some embodiments, nab-paclitaxel (ABRAXANE®) is administered at 100 mg/m² IV three times every four weeks (e.g., on Days 1, 8, and 15 of each 28-day dosing cycle). In some embodiments, the taxane (e.g., paclitaxel or nab-paclitaxel (ABRAXANE®)) may be administered weekly, every 2 weeks, every 3 weeks, every 4 weeks, on days 1, 8 and 15 of each 21-day cycle, or on days 1, 8, and 15 of each 28-day cycle.

In some embodiments, the taxane (e.g., paclitaxel or nab-paclitaxel (ABRAXANE®)) is administered to the subject or population of subjects intravenously (e.g., over a 3-hour infusion). In some embodiments, the taxane is administered to the subject or population of subjects every three weeks. In some embodiments, the taxane is administered to the subject or population of subjects on about Day 1 (e.g., Day −3, Day −2, Day −1, Day 1, Day 2, or Day 3) of a 21-day dosing cycle. In some embodiments, paclitaxel is administered to the subject every three weeks. In some embodiments, paclitaxel is administered to the subject or population of subjects on about Day 1 of a 21-day dosing cycle. In some embodiments, nab-paclitaxel (ABRAXANE®) is administered to the subject or population of subjects every three weeks. In some embodiments, nab-paclitaxel (ABRAXANE®) is administered to the subject or population of subjects on about Day 1 of a 21-day dosing cycle.

Antimetabolites

In some instances, the effective amount of an antimetabolite (e.g., pemetrexed or gemcitabine) administered as part of the methods described herein is from 10-10000 mg/m² (e.g., from 20-8000 mg/m², from 30-5000 mg/m², from 40-2500 mg/m², from 50-2000 mg/m², from 100-1500 mg/m², or from 400-1200 mg/m², e.g., about 20 mg/m², about 30 mg/m², about 40 mg/m², about 50 mg/m², about 60 mg/m², about 70 mg/m², about 80 mg/m², about 90 mg/m², about 100 mg/m², about 110 mg/m², about 120 mg/m², about 130 mg/m², about 140 mg/m², about 150 mg/m², about 160 mg/m², about 170 mg/m², about 180 mg/m², about 190 mg/m², about 200 mg/m², about 250 mg/m², about 300 mg/m², about 400 mg/m², about 500 mg/m², about 600 mg/m², about 700 mg/m², about 800 mg/m², about 900 mg/m², about 1000 mg/m², about 1100 mg/m², about 1200 mg/m², about 1250 mg/m², about 1300 mg/m², about 1400 mg/m², about 1500 mg/m², about 1750 mg/m², about 2000 mg/m², about 3000 mg/m², about 4000 mg/m², about 5000 mg/m², about 6000 mg/m², about 7000 mg/m², about 8000 mg/m², about 9000 mg/m², or about 10000 mg/m²). In some instances, the effective amount of the antimetabolite (e.g., pemetrexed or gemcitabine) is between about 500 mg/m² to about 1250 mg/m² (e.g., 20 mg/m², 30 mg/m², 40 mg/m², 50 mg/m², 60 mg/m², 70 mg/m², 80 mg/m², 90 mg/m², 100 mg/m², 110 mg/m², 120 mg/m², 130 mg/m², 140 mg/m², 150 mg/m², 160 mg/m², 170 mg/m², 180 mg/m², 190 mg/m², 200 mg/m², 250 mg/m², 300 mg/m², 400 mg/m², 500 mg/m², 600 mg/m², 700 mg/m², 800 mg/m², 900 mg/m², 1000 mg/m², 1100 mg/m², 1200 mg/m², 1250 mg/m², 1300 mg/m², 1400 mg/m², 1500 mg/m², 1750 mg/m², 2000 mg/m², 3000 mg/m², 4000 mg/m², 5000 mg/m², 6000 mg/m², 7000 mg/m², 8000 mg/m², 9000 mg/m², or 10000 mg/m²). In some instances, the effective amount of the antimetabolite (e.g., pemetrexed or gemcitabine) is about 500 mg/m². In some instances, the effective amount of the antimetabolite (e.g., pemetrexed or gemcitabine) is about 1000 mg/m². In some instances, the effective amount of the antimetabolite (e.g., pemetrexed or gemcitabine) is about 1250 mg/m². In some instances, the effective amount of the antimetabolite (e.g., pemetrexed or gemcitabine) is 500 mg/m². In some instances, the effective amount of the antimetabolite (e.g., pemetrexed or gemcitabine) is 1000 mg/m². In some instances, the effective amount of the antimetabolite (e.g., pemetrexed or gemcitabine) is 1250 mg/m².

In some instances, the effective amount of pemetrexed administered as part of the methods described herein is from 10-1000 mg/m² (e.g., from 20-900 mg/m², from 30-800 mg/m², from 40-700 mg/m², from 50-650 mg/m², from 100-600 mg/m², or from 200-550 mg/m², e.g., about 20 mg/m², about 30 mg/m², about 40 mg/m², about 50 mg/m², about 60 mg/m², about 70 mg/m², about 80 mg/m², about 90 mg/m², about 100 mg/m², about 110 mg/m², about 120 mg/m², about 130 mg/m², about 140 mg/m², about 150 mg/m², about 160 mg/m², about 170 mg/m², about 180 mg/m², about 190 mg/m², about 200 mg/m², about 250 mg/m², about 300 mg/m², about 400 mg/m², about 500 mg/m², about 600 mg/m², about 700 mg/m², about 800 mg/m², about 900 mg/m², or about 1000 mg/m²). In some instances, the effective amount of pemetrexed is about 500 mg/m². In some instances, the effective amount of pemetrexed is about 500 mg/m² every three weeks. In some instances, the effective amount of pemetrexed is 500 mg/m². In some instances, the effective amount of pemetrexed is 500 mg/m² every three weeks.

In some embodiments, the pemetrexed is administered to the subject or population of subjects intravenously (e.g., over a 10-minute infusion). In some embodiments, the pemetrexed is administered to the subject or population of subjects every three weeks. In some embodiments, the pemetrexed is administered to the subject or population of subjects on about Day 1 (e.g., Day −3, Day −2, Day −1, Day 1, Day 2, or Day 3) of a 21-day dosing cycle.

In some instances, the effective amount of gemcitabine administered as part of the methods described herein is from 10-10000 mg/m² (e.g., from 20-8000 mg/m², from 30-5000 mg/m², from 40-2500 mg/m², from 50-2000 mg/m², from 100-1500 mg/m², or from 400-1250 mg/m², e.g., about 20 mg/m², about 30 mg/m², about 40 mg/m², about 50 mg/m², about 60 mg/m², about 70 mg/m², about 80 mg/m², about 90 mg/m², about 100 mg/m², about 110 mg/m², about 120 mg/m², about 130 mg/m², about 140 mg/m², about 150 mg/m², about 160 mg/m², about 170 mg/m², about 180 mg/m², about 190 mg/m², about 200 mg/m², about 250 mg/m², about 300 mg/m², about 400 mg/m², about 500 mg/m², about 600 mg/m², about 700 mg/m², about 800 mg/m², about 900 mg/m², about 1000 mg/m², about 1100 mg/m², about 1200 mg/m², about 1250 mg/m², about 1300 mg/m², about 1400 mg/m², about 1500 mg/m², about 1750 mg/m², about 2000 mg/m², about 3000 mg/m², about 4000 mg/m², about 5000 mg/m², about 6000 mg/m², about 7000 mg/m², about 8000 mg/m², about 9000 mg/m², or about 10000 mg/m² (e.g., 20 mg/m², 30 mg/m², 40 mg/m², 50 mg/m², 60 mg/m², 70 mg/m², 80 mg/m², 90 mg/m², 100 mg/m², 110 mg/m², 120 mg/m², 130 mg/m², 140 mg/m², 150 mg/m², 160 mg/m², 170 mg/m², 180 mg/m², 190 mg/m², 200 mg/m², 250 mg/m², 300 mg/m², 400 mg/m², 500 mg/m², 600 mg/m², 700 mg/m², 800 mg/m², 900 mg/m², 1000 mg/m², 1100 mg/m², 1200 mg/m², 1250 mg/m², 1300 mg/m², 1400 mg/m², 1500 mg/m², 1750 mg/m², 2000 mg/m², 3000 mg/m², 4000 mg/m², 5000 mg/m², 6000 mg/m², 7000 mg/m², 8000 mg/m², 9000 mg/m², or 10000 mg/m²)). In some instances, the effective amount of gemcitabine is between about 500 mg/m² to about 1250 mg/m². In some instances, the effective amount of gemcitabine is about 500 mg/m². In some instances, the effective amount of gemcitabine is about 1000 mg/m². In some instances, the effective amount of gemcitabine is about 1000 mg/m² when given before carboplatin. In some instances, the effective amount of gemcitabine is about 1250 mg/m². In some instances, the effective amount of gemcitabine is about 1250 mg/m² when given before cisplatin. In some instances, the effective amount of gemcitabine is about 1000 mg/m² on Days 1 and 8 of a 21-day dosing cycle. In some instances, the effective amount of gemcitabine is about 1250 mg/m² on Days 1 and 8 of a 21-day dosing cycle. In some instances, the effective amount of gemcitabine is 1000 mg/m². In some instances, the effective amount of gemcitabine is 1000 mg/m² when given before carboplatin. In some instances, the effective amount of gemcitabine is 1250 mg/m². In some instances, the effective amount of gemcitabine is 1250 mg/m² when given before cisplatin. In some instances, the effective amount of gemcitabine is 1000 mg/m² on Days 1 and 8 of a 21-day dosing cycle. In some instances, the effective amount of gemcitabine is 1250 mg/m² on Days 1 and 8 of a 21-day dosing cycle.

In some embodiments, the gemcitabine is administered to the subject or population of subjects intravenously (e.g., over a 30-minute infusion). In some instances, the gemcitabine is administered to the subject or population of subjects on about Day 1 (e.g., Day −3, Day −2, Day −1, Day 1, Day 2, or Day 3) of a 21-day dosing cycle. In some instances, the gemcitabine is administered to the subject or population of subjects on about Day 1 and about Day 8 (e.g., Day 5, Day 6, Day 7, Day 8, Day 9, Day 10, or Day 11) of a 21-day dosing cycle.

### Topoisomerase II Inhibitors

In some instances, the effective amount of a topoisomerase II inhibitor (e.g., etoposide) is from 10-1000 mg/m² (e.g., from 20-800 mg/m², from 30-700 mg/m², from 40-500 mg/m², from 50-300 mg/m², from 75-200 mg/m², or from 80-150 mg/m², e.g., about 20 mg/m², about 30 mg/m², about 40 mg/m², about 50 mg/m², about 60 mg/m², about 70 mg/m², about 80 mg/m², about 90 mg/m², about 100 mg/m², about 110 mg/m², about 120 mg/m², about 130 mg/m², about 140 mg/m², about 150 mg/m², about 160 mg/m², about 170 mg/m², about 180 mg/m², about 190 mg/m², about 200 mg/m², about 250 mg/m², about 300 mg/m², about 400 mg/m², about 500 mg/m², about 600 mg/m², about 700 mg/m², about 800 mg/m², about 900 mg/m², or about 1000 mg/m² (e.g., 20 mg/m², 30 mg/m², 40 mg/m², 50 mg/m², 60 mg/m², 70 mg/m², 80 mg/m², 90 mg/m², 100 mg/m², 110 mg/m², 120 mg/m², 130 mg/m², 140 mg/m², 150 mg/m², 160 mg/m², 170 mg/m², 180 mg/m², 190 mg/m², 200 mg/m², 250 mg/m², 300 mg/m², 400 mg/m², 500 mg/m², 600 mg/m², 700 mg/m², 800 mg/m², 900 mg/m², or 1000 mg/m²)). In some instances, the effective amount of the topoisomerase II inhibitor (e.g., etoposide) is about 100 mg/m². In some instances, the effective amount of the topoisomerase II inhibitor (e.g., etoposide) is about 100 mg/m² on Days 1-3 every three weeks. In some instances, the effective amount of the topoisomerase II inhibitor (e.g., etoposide) is 100 mg/m² on Days 1-3 every three weeks.

In some embodiments, the topoisomerase II inhibitor (e.g., etoposide) is administered to the subject intravenously (e.g., over a 60-minute infusion).

In some instances, the effective amount of a topoisomerase II inhibitor (e.g., doxorubicin) is from 10-1000 mg/m² (e.g., from 20-800 mg/m², from 30-700 mg/m², from 40-500 mg/m², from 50-300 mg/m², from 75-200 mg/m², or from 80-150 mg/m², e.g., about 20 mg/m², about 30 mg/m², about 40 mg/m², about 50 mg/m², about 60 mg/m², about 70 mg/m², about 80 mg/m², about 90 mg/m², about 100 mg/m², about 110 mg/m², about 120 mg/m², about 130 mg/m², about 140 mg/m², about 150 mg/m², about 160 mg/m², about 170 mg/m², about 180 mg/m², about 190 mg/m², about 200 mg/m², about 250 mg/m², about 300 mg/m², about 400 mg/m², about 500 mg/m², about 600 mg/m², about 700 mg/m², about 800 mg/m², about 900 mg/m², or about 1000 mg/m² (e.g., 20 mg/m², 30 mg/m², 40 mg/m², 50 mg/m², 60 mg/m², 70 mg/m², 80 mg/m², 90 mg/m², 100 mg/m², 110 mg/m², 120 mg/m², 130 mg/m², 140 mg/m², 150 mg/m², 160 mg/m², 170 mg/m², 180 mg/m², 190 mg/m², 200 mg/m², 250 mg/m², 300 mg/m², 400 mg/m², 500 mg/m², 600 mg/m², 700 mg/m², 800 mg/m², 900 mg/m², or 1000 mg/m²)). In some instances, the effective amount of the topoisomerase II inhibitor (e.g., doxorubicin) is about 60 mg/m². In some instances, the effective amount of the topoisomerase II inhibitor (e.g., doxorubicin) is 60 mg/m². In some instances, the effective amount of the topoisomerase II inhibitor (e.g., doxorubicin) is about 100 mg/m². In some instances, the effective amount of the topoisomerase II inhibitor (e.g., doxorubicin) is 100 mg/m². In some instances, the dose of the topoisomerase II inhibitor (e.g., doxorubicin) is reduced or delayed to avoid toxicity.

In some embodiments, the topoisomerase II inhibitor (e.g., doxorubicin) may be administered weekly, every 2 weeks, every 3 weeks, every 4 weeks, on days 1, 8 and 15 of each 21-day cycle, or on days 1, 8, and 15 of each 28-day cycle. In some embodiments, the platinum-based chemotherapeutic agent (e.g., carboplatin or cisplatin) may be administered every 2 weeks.

In some embodiments, the alkylating agent (e.g., cyclophosphamide) is administered as an IV bolus over 3-5 minutes to the subject. In some embodiments, the alkylating agent (e.g., cyclophosphamide) is administered as an IV infusion over 15-30 minutes. In some embodiments, the topoisomerase II inhibitor (e.g., doxorubicin) is administered to the subject intravenously (e.g., over a 60-minute infusion).

### Alkylating Agents

A therapeutically effective amount of an alkylating agent (e.g., cyclophosphamide) administered to a human will be in the range of about 400 to about 800 mg/m² (e.g., about 400 mg/m², about 425 mg/m², about 450 mg/m², about 475 mg/m², about 500 mg/m², about 525 mg/m², about 550 mg/m², about 575 mg/m², about 600 mg/m², about 625 mg/m², about 650 mg/m², about 675 mg/m², about 700 mg/m², about 725 mg/m², about 750 mg/m², about 775 mg/m², or about 800 mg/m² (e.g., 400 mg/m², 425 mg/m², 450 mg/m², 475 mg/m², 500 mg/m², 525 mg/m², 550 mg/m², 575 mg/m², 600 mg/m², 625 mg/m², 650 mg/m², 675 mg/m², 700 mg/m², 725 mg/m², 750 mg/m², 775 mg/m², or 800 mg/m²)) whether by one or more administrations. For example, in some embodiments, about 600 mg/m² of an alkylating agent (e.g., cyclophosphamide) is administered. In some embodiments, about 600 mg/m² of an alkylating agent (e.g., cyclophosphamide) is administered every two weeks. In some embodiments, about 600 mg/m² of cyclophosphamide is administered. In some embodiments, about 600 mg/m² of cyclophosphamide is administered every two weeks. In some embodiments, 600 mg/m² of an alkylating agent (e.g., cyclophosphamide) is administered every two weeks. In some embodiments, the alkylating agent (e.g., cyclophosphamide) may be administered weekly, every 2 weeks, every 3 weeks, every 4 weeks, on days 1, 8 and 15 of each 21-day cycle, on days 1 and 22 of each 28-day cycle, or on days 1, 8, and 15 of each 28-day cycle.

In some embodiments, the alkylating agent (e.g., cyclophosphamide) is administered as an IV bolus over 3-5 minutes to the subject. In some embodiments, the alkylating agent (e.g., cyclophosphamide) is administered as an IV infusion.

Dosing of Colony Stimulating Factors

A therapeutically effective amount of a colony stimulating factor (CSF) (e.g., G-CSF (e.g., pegfilgrastim or filgrastim) and GM-CSF (e.g., sargramostim)) administered to a human will be in the range of about 1 to about 100 mg (e.g., about 1 mg, about 2 mg, about 3 mg, about 4 mg, about 5 mg, about 6 mg, about 7 mg, about 8 mg, about 9 mg, about 10 mg, about 15 mg, about 20 mg, about 25 mg, about 30 mg, about 45 mg, about 50 mg, about 60 mg, about 70 mg, about 80 mg, about 90 mg, or about 100 mg). For example, in some embodiments, about 6 mg is administered. In some aspects, a therapeutically effective amount of a colony stimulating factor (CSF) (e.g., G-CSF (e.g., pegfilgrastim or filgrastim) and GM-CSF (e.g., sargramostim)) administered to a human will be in the range of 1 to 100 mg (e.g., 1 mg, 2 mg, 3 mg, 4 mg, 5 mg, 6 mg, 7 mg, 8 mg, 9 mg, 10 mg, 15 mg, 20 mg, 25 mg, 30 mg, 45 mg, 50 mg, 60 mg, 70 mg, 80 mg, 90 mg, or 100 mg). In some embodiments, 6 mg of pegfilgrastim is administered. In some embodiments, the colony stimulating factor is pegfilgrastim. In some embodiments, the colony stimulating factor is filgrastim. In some embodiments, about 6 mg of pegfilgrastim is administered.

A therapeutically effective amount of a colony stimulating factor (e.g., G-CSF (e.g., pegfilgrastim or filgrastim) and GM-CSF (e.g., sargramostim)) administered to a human will be in the range of about 1 to about 400 mcg/kg/day (e.g., about 1 mcg/kg/day, about 2 mcg/kg/day, about 3 mcg/kg/day, about 4 mcg/kg/day, about 5 mcg/kg/day, about 6 mcg/kg/day, about 7 mcg/kg/day, about 8 mcg/kg/day, about 9 mcg/kg/day, about 10 mcg/kg/day, about 15 mcg/kg/day, about 20 mcg/kg/day, about 25 mcg/kg/day, about 50 mcg/kg/day, about 75 mcg/kg/day, about 100 mcg/kg/day, about 125 mcg/kg/day, about 150 mcg/kg/day, about 175 mcg/kg/day, about 200 mcg/kg/day, about 225 mcg/kg/day, about 250 mcg/kg/day, about 275 mcg/kg/day, about 300 mcg/kg/day, about 325 mcg/kg/day, about 350 mcg/kg/day, about 375 mcg/kg/day, or about 400 mcg/kg/day). For example, in some embodiments, about 250 mcg/m²/day is administered. In some embodiments, a therapeutically effective amount of a colony stimulating factor (e.g., G-CSF (e.g., pegfilgrastim or filgrastim) and GM-CSF (e.g., sargramostim)) administered to a human will be in the range of 1 to 400 mcg/kg/day (e.g., 1 mcg/kg/day, 2 mcg/kg/day, 3 mcg/kg/day, 4 mcg/kg/day, 5 mcg/kg/day, 6 mcg/kg/day, 7 mcg/kg/day, 8 mcg/kg/day, 9 mcg/kg/day, 10 mcg/kg/day, 15 mcg/kg/day, 20 mcg/kg/day, 25 mcg/kg/day, 50 mcg/kg/day, 75 mcg/kg/day, 100 mcg/kg/day, 125 mcg/kg/day, 150 mcg/kg/day, 175 mcg/kg/day, 200 mcg/kg/day, 225 mcg/kg/day, 250 mcg/kg/day, 275 mcg/kg/day, 300 mcg/kg/day, 325 mcg/kg/day, 350 mcg/kg/day, 375 mcg/kg/day, or 400 mcg/kg/day). For example, in some embodiments, 250 mcg/m²/day is administered. In some embodiments, the colony stimulating factor (e.g., G-CSF (e.g., pegfilgrastim or filgrastim) and GM-CSF (e.g., sargramostim)) is administered as a subcutaneous injection. In some embodiments, the colony stimulating factor is sargramostim. In some embodiments, sargramostim is administered to the human at a dose of about 250 mcg/m2/day. In some embodiments, sargramostim is administered to the human at a dose of 250 mcg/m2/day.

Dosing of VEGF Antagonists

In some instances, the effective amount of the VEGF antagonist (e.g., an anti-VEGF antibody (e.g., bevacizumab)) is a dose of between about 0.01 mg/kg to about 50 mg/kg of the subject's body weight (e.g., between about 0.01 mg/kg to about 45 mg/kg, e.g., between about 0.1 mg/kg to about 40 mg/kg, e.g., between about 1 mg/kg to about 35 mg/kg, e.g., between about 2.5 mg/kg to about 30 mg/kg, e.g., between about 5 mg/kg to about 25 mg/kg, e.g., between about 10 mg/kg to about 20 mg/kg, e.g., between about 12.5 mg/kg to about 15 mg/kg, e.g., about 15±2 mg/kg, about 15±1 mg/kg, about 15±0.5 mg/kg, about 15±0.2 mg/kg, or about 15±0.1 mg/kg, e.g., about 15 mg/kg) every three weeks (e.g., on Day 1 (e.g., Day 1±3 days) of each 21-day dosing cycle). In some instances, the effective amount of the VEGF antagonist (e.g., an anti-VEGF antibody (e.g., bevacizumab)) is a dose of between about 0.01 mg/kg to about 15 mg/kg of the subject's body weight (e.g., between about 0.1 mg/kg to about 15 mg/kg, e.g., between about 0.5 mg/kg to about 15 mg/kg, e.g., between about 1 mg/kg to about 15 mg/kg, e.g., between about 2.5 mg/kg to about 15 mg/kg, e.g., between about 5 mg/kg to about 15 mg/kg, e.g., between about 7.5 mg/kg to about 15 mg/kg, e.g., between about 10 mg/kg to about 15 mg/kg, e.g., between about 12.5 mg/kg to about 15 mg/kg, e.g., between about 14 mg/kg to about 15 mg/kg, e.g., about 15±1 mg/kg, e.g., about 15±0.5 mg/kg, e.g., about 15±0.2 mg/kg, e.g., about 15±0.1 mg/kg, e.g., about 15 mg/kg) every three weeks. In some instances, the effective amount of VEGF antagonist (e.g., an anti-VEGF antibody (e.g., bevacizumab)) is a dose of about 15 mg/kg administered every three weeks. In some instances, the effective amount of the VEGF antagonist (e.g., an anti-VEGF antibody (e.g., bevacizumab)) is a dose of between about 0.01 mg/kg to about 50 mg/kg of the subject's body weight (e.g., between about 0.01 mg/kg to about 45 mg/kg, e.g., between about 0.1 mg/kg to about 40 mg/kg, e.g., between about 1 mg/kg to about 35 mg/kg, e.g., between about 2.5 mg/kg to about 30 mg/kg, e.g., between about 5 mg/kg to about 25 mg/kg, e.g., between about 7.5 mg/kg to about 20 mg/kg, e.g., between about 7.5 mg/kg to about 15 mg/kg, e.g., between about 7.5 mg/kg to about 12.5 mg/kg, e.g., about 10±2 mg/kg, about 10±1 mg/kg, about 10±0.5 mg/kg, about 10±0.2 mg/kg, or about 10±0.1 mg/kg, e.g., about 10 mg/kg) every two weeks (e.g., on Day 1 (e.g., Day 1±3 days) of each 14-day dosing cycle or on Day 1 (e.g., Day 1±3 days) and Day 15 (e.g., Day 15±3 days) of each 28-day dosing cycle). In some instances, the effective amount of the VEGF antagonist (e.g., an anti-VEGF antibody (e.g., bevacizumab)) is a dose of between about 0.01 mg/kg to about 15 mg/kg of the subject's body weight (e.g., between about 0.1 mg/kg to about 15 mg/kg, e.g., between about 0.5 mg/kg to about 15 mg/kg, e.g., between about 1 mg/kg to about 15 mg/kg, e.g., between about 2.5 mg/kg to about 15 mg/kg, e.g., between about 5 mg/kg to about 15 mg/kg, e.g., between about 7.5 mg/kg to about 12.5 mg/kg, e.g., between about 8 mg/kg to about 12 mg/kg, e.g., between about 9 mg/kg to about 11 mg/kg, e.g., between about 9.5 mg/kg to about 10.5 mg/kg, e.g., about 10±1 mg/kg, e.g., about 10±0.5 mg/kg, e.g., about 10±0.2 mg/kg, e.g., about 10±0.1 mg/kg, e.g., about 10 mg/kg) every two weeks. In some instances, the effective amount of VEGF antagonist (e.g., an anti-VEGF antibody (e.g., bevacizumab)) is a dose of about 10 mg/kg administered every two weeks. In some instances, the effective amount of VEGF antagonist (e.g., an anti-VEGF antibody (e.g., bevacizumab)) is a dose of about 5 mg/kg administered every two weeks. In some instances, the effective amount of VEGF antagonist (e.g., an anti-VEGF antibody (e.g., bevacizumab)) is a dose of about 7.5 mg/kg administered every two weeks.

In some instances, the effective amount of the VEGF antagonist (e.g., an anti-VEGF antibody (e.g., bevacizumab)) is a dose of between 0.01 mg/kg to 50 mg/kg of the subject's body weight (e.g., between 0.01 mg/kg to 45 mg/kg, e.g., between 0.1 mg/kg to 40 mg/kg, e.g., between 1 mg/kg to 35 mg/kg, e.g., between 2.5 mg/kg to 30 mg/kg, e.g., between 5 mg/kg to 25 mg/kg, e.g., between 10 mg/kg to 20 mg/kg, e.g., between 12.5 mg/kg to 15 mg/kg, e.g., 15±2 mg/kg, 15±1 mg/kg, 15±0.5 mg/kg, 15±0.2 mg/kg, or 15±0.1 mg/kg, e.g., 15 mg/kg) every three weeks. In some instances, the effective amount of the VEGF antagonist (e.g., an anti-VEGF antibody (e.g., bevacizumab)) is a dose of between 0.01 mg/kg to 15 mg/kg of the subject's body weight (e.g., between 0.1 mg/kg to 15 mg/kg, e.g., between 0.5 mg/kg to 15 mg/kg, e.g., between 1 mg/kg to 15 mg/kg, e.g., between 2.5 mg/kg to 15 mg/kg, e.g., between 5 mg/kg to 15 mg/kg, e.g., between 7.5 mg/kg to 15 mg/kg, e.g., between 10 mg/kg to 15 mg/kg, e.g., between 12.5 mg/kg to 15 mg/kg, e.g., between 14 mg/kg to 15 mg/kg, e.g., 15±1 mg/kg, e.g., 15+0.5 mg/kg, e.g., 15±0.2 mg/kg, e.g., 15±0.1 mg/kg, e.g., 15 mg/kg) every three weeks. In some instances, the effective amount of VEGF antagonist (e.g., an anti-VEGF antibody (e.g., bevacizumab)) is a dose of 15 mg/kg administered every three weeks. In some instances, the effective amount of the VEGF antagonist (e.g., an anti-VEGF antibody (e.g., bevacizumab)) is a dose of between 0.01 mg/kg to 50 mg/kg of the subject's body weight (e.g., between 0.01 mg/kg to 45 mg/kg, e.g., between 0.1 mg/kg to 40 mg/kg, e.g., between 1 mg/kg to 35 mg/kg, e.g., between 2.5 mg/kg to 30 mg/kg, e.g., between 5 mg/kg to 25 mg/kg, e.g., between 7.5 mg/kg to 20 mg/kg, e.g., between 7.5 mg/kg to 15 mg/kg, e.g., between 7.5 mg/kg to 12.5 mg/kg, e.g., 10 2 mg/kg, 10±1 mg/kg, 10±0.5 mg/kg, 10±0.2 mg/kg, or 10±0.1 mg/kg, e.g., 10 mg/kg) every two weeks. In some instances, the effective amount of the VEGF antagonist (e.g., an anti-VEGF antibody (e.g., bevacizumab)) is a dose of between 0.01 mg/kg to 15 mg/kg of the subject's body weight (e.g., between 0.1 mg/kg to 15 mg/kg, e.g., between 0.5 mg/kg to 15 mg/kg, e.g., between 1 mg/kg to 15 mg/kg, e.g., between 2.5 mg/kg to 15 mg/kg, e.g., between 5 mg/kg to 15 mg/kg, e.g., between 7.5 mg/kg to 12.5 mg/kg, e.g., between 8 mg/kg to 12 mg/kg, e.g., between 9 mg/kg to 11 mg/kg, e.g., between 9.5 mg/kg to 10.5 mg/kg, e.g., 10±1 mg/kg, e.g., 10 0.5 mg/kg, e.g., 10±0.2 mg/kg, e.g., 10±0.1 mg/kg, e.g., 10 mg/kg) every two weeks. In some instances, the effective amount of VEGF antagonist (e.g., an anti-VEGF antibody (e.g., bevacizumab)) is a dose of 10 mg/kg administered every two weeks. In some instances, the effective amount of VEGF antagonist (e.g., an anti-VEGF antibody (e.g., bevacizumab)) is a dose of 5 mg/kg administered every two weeks. In some instances, the effective amount of VEGF antagonist (e.g., an anti-VEGF antibody (e.g., bevacizumab)) is a dose of 7.5 mg/kg administered every two weeks.

In some instances, the dose of the VEGF antagonist (e.g., an anti-VEGF antibody (e.g., bevacizumab)) administered in a combination therapy (e.g., a combination treatment with an anti-TIGIT antagonist antibody, such as an anti-TIGIT antagonist antibody disclosed herein (e.g., tiragolumab), and/or a PD-1 axis binding antagonist (e.g., anti-PD-L1 antagonist antibody (e.g., atezolizumab) or anti-PD-1 antagonist antibody (e.g., pembrolizumab)) may be reduced as compared to a standard dose of the PD-1 axis binding antagonist administered as a monotherapy.

In some instances, the VEGF antagonist (e.g., an anti-VEGF antibody (e.g., bevacizumab)) is administered intravenously. In some instances, the VEGF antagonist (e.g., an anti-VEGF antibody (e.g., bevacizumab)) is administered intravenously over 90±15 minutes (e.g., about 75 minutes, about 76 minutes, about 77 minutes, about 78 minutes, about 79 minutes, about 80 minutes, about 81 minutes, about 82 minutes, about 83 minutes, about 84 minutes, about 85 minutes, about 86 minutes, about 87 minutes, about 88 minutes, about 89 minutes, about 90 minutes, about 91 minutes, about 92 minutes, about 93 minutes, about 94 minutes, about 95 minutes, about 96 minutes, about 97 minutes, about 98 minutes, about 99 minutes, about 100 minutes, about 101 minutes, about 102 minutes, about 103 minutes, about 104 minutes, or about 105 minutes). Alternatively, in some embodiments, the VEGF antagonist (e.g., an anti-VEGF antibody (e.g., bevacizumab)) is administered subcutaneously.

L. Assessment of PD-L1 Expression

The expression of PD-L1 may be assessed in a subject treated according to any of the methods, uses, and compositions for use described herein. The methods, uses, and compositions for use may include determining the expression level of PD-L1 in a biological sample (e.g., a tumor sample) obtained from the subject. In some instances, the patient has a bladder cancer and the sample is a transurethral resection of bladder tumor (TURBT) sample. In some instances, the sample is a cystectomy or nephroureterectomy sample. In some instances, the sample is a segmentectomy, a lobectomy, a bilobectomy, or a pneumonectomy sample. In some instances, the sample is a lymph node dissection sample. In other examples, the expression level of PD-L1 in a biological sample (e.g., a tumor sample) obtained from the subject has been determined prior to initiation of treatment or after initiation of treatment. PD-L1 expression may be determined using any suitable approach. For example, PD-L1 expression may be determined as described in U.S. Patent Application Nos. US20180030138A1 and US20180037655A1. Any suitable tumor sample may be used, e.g., a formalin-fixed and paraffin-embedded (FFPE) tumor sample, an archival tumor sample, a fresh tumor sample, or a frozen tumor sample.

For example, PD-L1 expression may be determined in terms of the percentage of a tumor sample comprised by tumor-infiltrating immune cells expressing a detectable expression level of PD-L1, as the percentage of tumor-infiltrating immune cells in a tumor sample expressing a detectable expression level of PD-L1, and/or as the percentage of tumor cells in a tumor sample expressing a detectable expression level of PD-L1. It is to be understood that in any of the preceding examples, the percentage of the tumor sample comprised by tumor-infiltrating immune cells may be in terms of the percentage of tumor area covered by tumor-infiltrating immune cells in a section of the tumor sample obtained from the subject, for example, as assessed by IHC using an anti-PD-L1 antibody (e.g., the SP142 antibody). Any suitable anti-PD-L1 antibody may be used, including, e.g., SP142 (Ventana), SP263 (Ventana), 22C3 (Dako), 28-8 (Dako), E1 L3N (Cell Signaling Technology), 4059 (ProSci, Inc.), h5H1 (Advanced Cell Diagnostics), and 9A11. In some examples, the anti-PD-L1 antibody is SP142. In other examples, the anti-PD-L1 antibody is SP263. In some examples, the anti-PD-L1 antibody is 22C3. In some examples, the anti-PD-L1 antibody is 28-8.

In some examples, a tumor sample obtained from the subject has a detectable expression level of PD-L1 in less than 1% of the tumor cells in the tumor sample, in 1% or more of the tumor cells in the tumor sample, in from 1% to less than 5% of the tumor cells in the tumor sample, in 5% or more of the tumor cells in the tumor sample, in from 5% to less than 50% of the tumor cells in the tumor sample, or in 50% or more of the tumor cells in the tumor sample.

In some examples, a tumor sample obtained from the subject has a detectable expression level of PD-L1 in tumor-infiltrating immune cells that comprise less than 1% of the tumor sample, more than 1% of the tumor sample, from 1% to less than 5% of the tumor sample, more than 5% of the tumor sample, from 5% to less than 10% of the tumor sample, or more than 10% of the tumor sample.

In some aspects, a tumor sample obtained from the subject has a detectable expression level of PD-L1 in tumor-infiltrating immune cells that comprise 5%-19% of the tumor sample (e.g., TIC 5%-19%); e.g., has a PD-L1 expression level that is PD-L1 low. In some aspects, a tumor sample obtained from the subject has a detectable expression level of PD-L1 in tumor-infiltrating immune cells that comprise ≥20% of the tumor sample (e.g., TIC ≥20%); e.g., has a PD-L1 expression level that is PD-L1 high. In some embodiments, tumor samples that have been determined to have a TIC of greater than, or equal to, 5% are comparable to a CPS of greater than, or equal to, 1.

In some examples, tumor samples may be scored for PD-L1 positivity in tumor-infiltrating immune cells and/or in tumor cells according to the criteria for diagnostic assessment shown in Table 1 and/or Table 2, respectively.

TABLE 1
Tumor-infiltrating immune cell (IC) IHC diagnostic criteria

| PD-L1 Diagnostic Assessment | IC Score |
|---|---|
| Absence of any discernible PD-L1 staining | IC0 |
| OR | |
| Presence of discernible PD-L1 staining of any intensity in tumor-infiltrating immune | |
| cells covering <1% of tumor area occupied by tumor cells, associated intratumoral | |
| stroma, and contiguous peri-tumoral desmoplastic stroma | |
| Presence of discernible PD-L1 staining of any intensity in tumor-infiltrating immune | IC1 |
| cells covering ≥1% to <5% of tumor area occupied by tumor cells, associated | |
| intratumoral stroma, and contiguous peri-tumoral desmoplastic stroma | |
| Presence of discernible PD-L1 staining of any intensity in tumor-infiltrating immune | IC2 |
| cells covering ≥5% to <10% of tumor area occupied by tumor cells, associated | |
| intratumoral stroma, and contiguous peri-tumoral desmoplastic stroma | |
| Presence of discernible PD-L1 staining of any intensity in tumor-infiltrating immune | IC3 |
| cells covering ≥10% of tumor area occupied by tumor cells, associated intratumoral | |
| stroma, and contiguous peri-tumoral desmoplastic stroma | |

TABLE 2
Tumor cell (TC) IHC diagnostic criteria

| PD-L1 Diagnostic Assessment | TC Score |
|---|---|
| Absence of any discernible PD-L1 staining | TC0 |
| OR | |
| Presence of discernible PD-L1 staining of any intensity in <1% of tumor cells | |
| Presence of discernible PD-L1 staining of any intensity in ≥1% to <5% of tumor cells | TC1 |
| Presence of discernible PD-L1 staining of any intensity in ≥5% to <50% of tumor cells | TC2 |
| Presence of discernible PD-L1 staining of any intensity in ≥50% of tumor cells | TC3 |

In some instances, in any of the methods, uses, or compositions for use described herein, the subject has a PD-L1 selected tumor (e.g., a proportion of tumor area occupied by PD-L1 expressing tumor-infiltrating immune cells (ICs) is greater than or equal to 5% in the tumor sample as determined by an IHC with the SP142 antibody). In some instances, the PD-L1 selected tumor is a tumor that has been determined to have a proportion of tumor area occupied by PD-L1 expressing immune cells (ICs) greater than or equal to 5% by an immunohistochemical (IHC) assay. In some instances, the IHC assay uses the anti-PD-L1 antibody SP142, SP263, 22C3, or 28-8. In some instances, the IHC assay uses anti-PD-L1 antibody SP142. In some instances, the IHC assay uses anti-PD-L1 antibody SP263. In some instances, the IHC assay uses anti-PD-L1 antibody 22C3. In some instances, the IHC assay uses anti-PD-L1 antibody 22C3. In some instances, the IHC assay uses anti-PD-L1 antibody 28-8.

In some instances, the ICs has been determined to be greater than, or equal to, 5% (e.g., as determined using the Ventana (SP142) PD-L1 IHC assay). In some instances, the IC score has been determined to be 2 or 3 (e.g., as determined using the Ventana (SP142) PD-L1 IHC assay). In some instances, the ICs has been determined to be greater than, or equal to, 1% (e.g., as determined using the Ventana (SP142) PD-L1 IHC assay). In some instances, the ICs has been determined to be greater than, or equal to, 10% (e.g., as determined using the Ventana (SP142) PD-L1 IHC assay). In some instances, the ICs has been determined to be greater than, or equal to, 1% and less than 50% (e.g., as determined using the Ventana (SP142) PD-L1 IHC assay). In some instances, the ICs has been determined to be greater than, or equal to, 1% and less than 30% (e.g., as determined using the Ventana (SP142) PD-L1 IHC assay).

In some instances, in any of the methods, uses, or compositions for use described herein, a tumor sample obtained from the individual has a detectable protein expression level of PD-L1. In some instances, the detectable protein expression level of PD-L1 has been determined by an IHC assay. In some instances, the IHC assay uses anti-PD-L1 antibody SP142. In some instances, the tumor sample has been determined to have a detectable expression level of PD-L1 in tumor-infiltrating immune cells that comprise greater than, or equal to, 5% of the tumor sample. In some instances, the tumor sample has been determined to have a detectable expression level of PD-L1 in tumor-infiltrating immune cells that comprise greater than, or equal to, 1% of the tumor sample. In some instances, the tumor sample has been determined to have a detectable expression level of PD-L1 in tumor-infiltrating immune cells that comprise greater than, or equal to, 1% and less than 5% of the tumor sample. In some instances, the tumor sample has been determined to have a detectable expression level of PD-L1 in tumor-infiltrating immune cells that comprise greater than, or equal to, 5% and less than 10% of the tumor sample. In some instances, the tumor sample has been determined to have a detectable expression level of PD-L1 in tumor-infiltrating immune cells that comprise greater than, or equal to, 10% of the tumor sample. In some instances, the tumor sample has been determined to have a detectable expression level of PD-L1 in greater than, or equal to, 1% of the tumor cells in the tumor sample. In some instances, the tumor sample has been determined to have a detectable expression level of PD-L1 in greater than, or equal to, 1% and less than 5% of the tumor cells in the tumor sample. In some instances, the tumor sample has been determined to have a detectable expression level of PD-L1 in greater than, or equal to, 5% and less than 50% of the tumor cells in the tumor sample. In some instances, the tumor sample has been determined to have a detectable expression level of PD-L1 in greater than, or equal to, 50% of the tumor cells in the tumor sample.

In some instances, in any of the methods, uses, or compositions for use described herein, the subject has a PD-L1 selected tumor (e.g., a PD-L1 high (e.g., a PD-L1 tumor proportion score (TPS) greater than or equal to 50% in a tumor sample as determined by an IHC with the SP263 antibody). In some instances, the PD-L1 selected tumor is a PD-L1 high selected tumor. In some instances, the PD-L1 selected tumor is a tumor that has been determined to have TPS greater than or equal to 50% by an immunohistochemical (IHC) assay. In some instances, the IHC assay uses the anti-PD-L1 antibody SP263, SP142, 22C3, or 28-8. In some instances, the IHC assay uses anti-PD-L1 antibody SP263. In some instances, the IHC assay uses anti-PD-L1 antibody SP142. In some instances, the IHC assay uses anti-PD-L1 antibody 22C3. In some instances, the TPS has been determined to be greater than, or equal to, 50% (e.g., as determined using the Ventana (SP263) PD-L1 IHC assay). In some instances, the TPS has been determined to be less than 50% (e.g., as determined using the Ventana (SP263) PD-L1 IHC assay). In some instances, the TPS has been determined to be greater than, or equal to, 1% (e.g., as determined using the Ventana (SP263) PD-L1 IHC assay). In some instances, the TPS has been determined to be greater than, or equal to, 1% and less than 50% (e.g., as determined using the Ventana (SP263) PD-L1 IHC assay).

In some instances, in any of the methods, uses, or compositions for use described herein, a tumor sample obtained from the individual has a detectable protein expression level of PD-L1. In some instances, the detectable protein expression level of PD-L1 has been determined by an IHC assay. In some instances, the IHC assay uses anti-PD-L1 antibody SP263. In some instances, the tumor sample has been determined to have a PD-L1-positive tumor cell fraction greater than, or equal to, 50% of the tumor sample. In some instances, the tumor sample has been determined to have a PD-L1-positive tumor cell fraction less than 50% of the tumor sample. In some instances, the tumor sample has been determined to have a PD-L1-positive tumor cell fraction greater than, or equal to, 1% and less than 50% of the tumor sample.

In some instances, the IHC assay uses the anti-PD-L1 antibody 22C3. In some instances, the IHC assay is the pharmDx 22C3 IHC assay. In some instances, the PD-L1-positive tumor cell fraction is greater than, or equal to, 50% as determined by positive staining with the anti-PD-L1 antibody 22C3. In some embodiments, the tumor sample has been determined to have a combined positive score (CPS) of greater than, or equal to, 10 or a tumor proportion score (TPS) of greater than or equal to 1% in the tumor sample, e.g., as determined using the anti-PD-L1 antibody 22C3 as part of the pharmDx 22C3 IHC assay. In some embodiments, the tumor sample has been determined to have a CPS of greater than, or equal to, 10 or a TPS of greater than or equal to 1% and less than 50% in the tumor sample, e.g., as determined using the anti-PD-L1 antibody 22C3 as part of the pharmDx 22C3 IHC assay. In some embodiments, the tumor sample has been determined to have a CPS of greater than, or equal to, 20 or a TPS of greater than or equal to 50% in the tumor sample, e.g., as determined using the anti-PD-L1 antibody 22C3 as part of the pharmDx 22C3 IHC assay. In some embodiments, tumor samples that have been determined to have a CPS of greater than, or equal to, 1 is are comparable to a TIC of greater than, or equal to, 5%.

In some instances, the IHC assay uses the anti-PD-L1 antibody 28-8. In some instances, the IHC assay is the pharmDx 28-8 IHC assay. In some instances, the PD-L1-positive tumor cell fraction is greater than, or equal to, 50% as determined by positive staining with the anti-PD-L1 antibody 28-8.

In some instances, in any of the methods, uses, or compositions for use described herein, a tumor sample obtained from the individual has a detectable nucleic acid expression level of PD-L1. In some instances, the detectable nucleic acid expression level of PD-L1 has been determined by RNA-seq, RT-qPCR, qPCR, multiplex qPCR or RT-qPCR, microarray analysis, SAGE, MassARRAY technique, ISH, or a combination thereof. In some instances, the sample is selected from the group consisting of a tissue sample, a whole blood sample, a serum sample, and a plasma sample. In some instances, the tissue sample is a tumor sample. In some instances, the tumor sample comprises tumor-infiltrating immune cells, tumor cells, stromal cells, and any combinations thereof.

M. Assessment of TIGIT Expression

The expression of TIGIT may be assessed in a subject treated according to any of the methods, uses, and compositions for use described herein. The methods, uses, and compositions for use may include determining the expression level of TIGIT in a biological sample (e.g., a tumor sample) obtained from the subject. In other examples, the expression level of TIGIT in a biological sample (e.g., a tumor sample) obtained from the subject has been determined prior to initiation of treatment or after initiation of treatment. TIGIT expression may be determined using any suitable approach. Any suitable tumor sample may be used, e.g., a formalin-fixed and paraffin-embedded (FFPE) tumor sample, an archival tumor sample, a fresh tumor sample, or a frozen tumor sample.

For example, TIGIT expression may be determined in terms of the percentage of a tumor sample comprised by tumor-infiltrating immune cells expressing a detectable expression level of TIGIT, as the percentage of tumor-infiltrating immune cells in a tumor sample expressing a detectable expression level of TIGIT, and/or as the percentage of tumor cells in a tumor sample expressing a detectable expression level of TIGIT. It is to be understood that in any of the preceding examples, the percentage of the tumor sample comprised by tumor-infiltrating immune cells may be in terms of the percentage of tumor area covered by tumor-infiltrating immune cells in a section of the tumor sample obtained from the subject, for example, as assessed by IHC using an anti-TIGIT antagonist antibody. Any suitable anti-TIGIT antagonist antibody may be used. In some examples, the anti-TIGIT antagonist antibody is 10A7 (WO 2009/126688A3; U.S. Pat. No. 9,499,596).

N. Assessment of EGFR and ALK aberrations

In some instances, in any of the methods, uses, or compositions for use described herein, the subject has no epidermal growth factor receptor (EGFR) or anaplastic lymphoma kinase (ALK) genomic tumor aberrations. In some instances, in any of the methods, uses, or compositions for use described herein, the subject does not have an EGFR gene mutation (e.g., a sensitizing or activating EGFR gene mutation) or ALK gene rearrangement (e.g., ALK fusion oncogene). In some instances, the subject has an Eastern Cooperative Oncology Group (ECOG) Performance Status (PS) of 0 or 1.

Methods for detecting the mutational status EGFR and ALK are well known in the art, and include, but are not limited to, sequencing DNA from clinical samples (e.g., tumor biopsies or blood samples (e.g., circulating tumor DNA in blood)) using a next-generation sequencing method, such as the targeted gene pulldown and sequencing method described in Frampton et al. (Nature Biotechnology. 31(11): 1023-1033, 2013), which is incorporated by reference herein in its entirety. Such a next-generation sequencing method can be used with any of the methods disclosed herein to detect various mutations (e.g., insertions, deletions, base substitutions, focal gene amplifications, and/or homozygous gene deletions), while enabling the use of small samples (e.g., from small-core needle biopsies, fine-needle aspirations, and/or cell blocks) or fixed samples (e.g., formalin-fixed and paraffin-embedded (FFPE) samples). Other methods for the detection of the mutational status of EGFR and ALK include fluorescence in situ hybridization (FISH) and immunohistochemical (IHC) methods. Exemplary methods for the detection of the mutational status of ALK are disclosed in U.S. Pat. No. 9,651,555, which is herein incorporated by reference in its entirety. In some instances, the VENTANA® anti-ALK(D5F3) IHC assay is used to determine the mutational status of the ALK gene.

In some instances of any of the methods described herein, the mutation is a sensitizing EGFR mutation. Sensitizing EGFR mutations are well known in the art and include those described in U.S. Publication No: US 2018/0235968 and in Juan et al. (Therapeutic Advances in Medical Oncology. 9(3): 201-216, 2017), which are incorporated by reference herein in their entireties. In some instances, the sensitizing EGFR mutation is a mutation in any one of exons 18-21 (e.g., a mutation in exon 18, exon 19, exon 20, and/or exon 21). In some instances, the sensitizing EGFR mutation is a deletion of exon 19 (dell 9). In other instances, sensitizing EGFR mutation is a L858R point mutation in exon 21. In some instances, the sensitizing EGFR mutation is a G719X point mutation in exon 18, wherein “X” is most commonly C, A, or S. In some instances, the sensitizing EGFR mutation is a G719S point mutation in exon 18. In some instances, the sensitizing EGFR mutation is a G719A point mutation in exon 18. In some instances, the sensitizing EGFR mutation is a S720F point mutation in exon 18. In some instances, the sensitizing EGFR mutation is a L861Q point mutation in exon 21. In some instances, the sensitizing EGFR mutation is a L861R point mutation in exon 21. In other instances, the sensitizing EGFR mutation is a T790M point mutation. In some instances, the sensitizing EGFR mutation is an E709X point mutation, where “X” is most commonly K, A, or H. In some instances, the sensitizing EGFR mutation is a S768I point mutation.

In some instances of any of the methods described herein, the mutation is an ALK gene rearrangement. ALK gene rearrangements are well known in the art and include those described in U.S. Pat. No. 9,651,555 and in Du et al. (Thoracic Cancer. 9: 423-430, 2018), which are incorporated herein by reference in their entireties. In some instances, the ALK gene rearrangement results in the creation of an oncogenic ALK tyrosine kinase that activates downstream signaling pathways resulting in increased cell proliferation and survival. In some instances, the ALK gene rearrangement is an ALK rearrangement with a gene selected from the group consisting of EML4, KIF5B, KLC1, TFG, TPR, HIP1, STRN, DCTN1, SQSTM1, NPM1, BCL11A, BIRC6, RANBP2, AT/C, CLTC, TMP4, and MSN resulting in the formation of a fusion oncogene. In some instances, the ALK gene rearrangement is an EML4 rearrangement with ALK resulting in the formation of the fusion oncogene EML4-ALK.

### IV. Exemplary Anti-TIGIT Antagonist Antibodies, Pd-1 Axis Binding Antagonists, and Other Agents

Exemplary anti-TIGIT antagonist antibodies, PD-1 axis binding antagonists, chemotherapeutic agents, colony stimulating factors, and VEGF antagonists useful for treating a subject (e.g., a human) having a cancer in accordance with the methods, uses, and compositions for use of the invention are described herein.

### A. Exemplary Anti-TIGIT Antagonist Antibodies

The invention provides anti-TIGIT antagonist antibodies useful for treating cancer in a subject (e.g., a human).

In some instances, the anti-TIGIT antagonist antibody is tiragolumab (CAS Registry Number: 1918185-84-8). Tiragolumab (Genentech) is also known as MTIG7192A.

In certain instances, the anti-TIGIT antagonist antibody includes at least one, two, three, four, five, or six HVRs selected from: (a) an HVR-H1 comprising the amino acid sequence of SNSAAWN (SEQ ID NO: 1); (b) an HVR-H2 comprising the amino acid sequence of KTYYRFKWYSDYAVSVKG (SEQ ID NO: 2); (c) an HVR-H3 comprising the amino acid sequence of ESTTYDLLAGPFDY (SEQ ID NO: 3); (d) an HVR-L1 comprising the amino acid sequence of KSSQTVLYSSNNKKYLA (SEQ ID NO: 4), (e) an HVR-L2 comprising the amino acid sequence of WASTRES (SEQ ID NO: 5); and/or (f) an HVR-L3 comprising the amino acid sequence of QQYYSTPFT (SEQ ID NO: 6), or a combination of one or more of the above HVRs and one or more variants thereof having at least about 90% sequence identity (e.g., 90%, 91%, 92%, 93%, 94%, 95%, 96%, 97%, 98%, or 99% identity) to any one of SEQ ID NOs: 1-6.

In some instances, anti-TIGIT antagonist antibodies may include (a) an HVR-H1 comprising the amino acid sequence of SNSAAWN (SEQ ID NO: 1); (b) an HVR-H2 comprising the amino acid sequence of KTYYRFKWYSDYAVSVKG (SEQ ID NO: 2); (c) an HVR-H3 comprising the amino acid sequence of ESTTYDLLAGPFDY (SEQ ID NO: 3); (d) an HVR-L1 comprising the amino acid sequence of KSSQTVLYSSNNKKYLA (SEQ ID NO: 4); (e) an HVR-L2 comprising the amino acid sequence of WASTRES (SEQ ID NO: 5); and (f) an HVR-L3 comprising the amino acid sequence of QQYYSTPFT (SEQ ID NO: 6). In some instances, the anti-TIGIT antagonist antibody has a VH domain comprising an amino acid sequence having at least 90% sequence identity (e.g., at least 91%, 92%, 93%, 94%, 95%, 96%, 97%, 98%, or 99% sequence identity) to, or the sequence of,

(SEQ ID NO: 17)
EVQLQQSGPGLVKPSQTLSLTCAISGDSVSSNSAAWNWIRQSPSRGLEW
LGKTYYRFKWYSDYAVSVKGRITINPDTSKNQFSLQLNSVTPEDTAVFY
CTRESTTYDLLAGPFDYWGQGTLVTVSS

or an amino acid sequence having at least 90% sequence identity (e.g., at least 91%, 92%, 93%, 94%, 95%, 96%, 97%, 98%, or 99% sequence identity) to, or the sequence of,

(SEQ ID NO: 18)
QVQLQQSGPGLVKPSQTLSLTCAISGDSVSSNSAAWNWIRQSPSRGLE
WLGKTYYRFKWYSDYAVSVKGRITINPDTSKNQFSLQLNSVTPEDTAV
FYCTRESTTYDLLAGPFDYWGQGTLVTVSS;

and/or a VL domain comprising an amino acid sequence having at least 90% sequence identity (e.g., at least 91%, 92%, 93%, 94%, 95%, 96%, 97%, 98%, or 99% sequence identity) to, or the sequence of, DIVMTQSPDSLAVSLGERATINCKSSQTVLYSSNNKKYLAWYQQKPGQPPNLLIYWASTRESGVPDRFS GSGSGTDFTLTISSLQAEDVAVYYCQQYYSTPFTFGPGTKVEIK (SEQ ID NO: 19). In some instances, the anti-TIGIT antagonist antibody has a VH domain comprising an amino acid sequence having at least 90% sequence identity (e.g., at least 91%, 92%, 93%, 94%, 95%, 96%, 97%, 98%, or 99% sequence identity) to, or the sequence of, SEQ ID NO: 17 and/or a VL domain comprising an amino acid sequence having at least 90% sequence identity (e.g., at least 91%, 92%, 93%, 94%, 95%, 96%, 97%, 98%, or 99% sequence identity) to, or the sequence of, SEQ ID NO: 19. In some instances, the anti-TIGIT antagonist antibody has a VH domain comprising the amino acid sequence of SEQ ID NO: 17 and a VL domain comprising the amino acid sequence of SEQ ID NO: 19. In some instances, the anti-TIGIT antagonist antibody has a VH domain comprising an amino acid sequence having at least 90% sequence identity (e.g., at least 91%, 92%, 93%, 94%, 95%, 96%, 97%, 98%, or 99% sequence identity) to, or the sequence of, SEQ ID NO: 18 and/or a VL domain comprising an amino acid sequence having at least 90% sequence identity (e.g., at least 91%, 92%, 93%, 94%, 95%, 96%, 97%, 98%, or 99% sequence identity) to, or the sequence of, SEQ ID NO: 19. In some instances, the anti-TIGIT antagonist antibody has a VH domain comprising the amino acid sequence of SEQ ID NO: 18 and a VL domain comprising the amino acid sequence of SEQ ID NO: 19.

In some instances, the anti-TIGIT antagonist antibody includes a heavy chain and a light chain sequence, wherein: (a) the heavy chain comprises the amino acid sequence:

(SEQ ID NO: 33)
EVQLQQSGPGLVKPSQTLSLTCAISGDSVSSNSAAWNWIRQSPSRGLE
WLGKTYYRFKWYSDYAVSVKGRITINPDTSKNQFSLQLNSVTPEDTAV
FYCTRESTTYDLLAGPFDYWGQGTLVTVSSASTKGPSVFPLAPSSKST
SGGTAALGCLVKDYFPEPVTVSWNSGALTSGVHTFPAVLQSSGLYSLS
SVVTVPSSSLGTQTYICNVNHKPSNTKVDKKVEPKSCDKTHTCPPCPA
PELLGGPSVFLFPPKPKDTLMISRTPEVTCVVVDVSHEDPEVKFNWYV
DGVEVHNAKTKPREEQYNSTYRVVSVLTVLHQDWLNGKEYKCKVSNKA
LPAPIEKTISKAKGQPREPQVYTLPPSREEMTKNQVSLTCLVKGFYPS
DIAVEWESNGQPENNYKTTPPVLDSDGSFFLYSKLTVDKSRWQQGNVF
SCSVMHEALHNHYTQKSLSLSPGK;

and (b) the light chain comprises the amino acid sequence:

(SEQ ID NO: 34)
DIVMTQSPDSLAVSLGERATINCKSSQTVLYSSNNKKYLAWYQQKPGQ
PPNLLIYWASTRESGVPDRFSGSGSGTDFTLTISSLQAEDVAVYYCQQ
YYSTPFTFGPGTKVEIKRTVAAPSVFIFPPSDEQLKSGTASVVCLLNN
FYPREAKVQWKVDNALQSGNSQESVTEQDSKDSTYSLSSTLTLSKADY
EKHKVYACEVTHQGLSSPVTKSFNRGEC.

In some instances, the anti-TIGIT antagonist antibody further comprises at least one, two, three, or four of the following light chain variable region framework regions (FRs): an FR-L1 comprising the amino acid sequence of DIVMTQSPDSLAVSLGERATINC (SEQ ID NO: 7); an FR-L2 comprising the amino acid sequence of WYQQKPGQPPNLLIY (SEQ ID NO: 8); an FR-L3 comprising the amino acid sequence of GVPDRFSGSGSGTDFTLTISSLQAEDVAVYYC (SEQ ID NO: 9); and/or an FR-L4 comprising the amino acid sequence of FGPGTKVEIK (SEQ ID NO: 10), or a combination of one or more of the above FRs and one or more variants thereof having at least about 90% sequence identity (e.g., 90%, 91%, 92%, 93%, 94%, 95%, 96%, 97%, 98%, or 99% identity) to anyone of SEQ ID NOs: 7-10. In some instances, for example, the antibody further comprises an FR-L1 comprising the amino acid sequence of DIVMTQSPDSLAVSLGERATINC (SEQ ID NO: 7); an FR-L2 comprising the amino acid sequence of WYQQKPGQPPNLLIY (SEQ ID NO: 8); an FR-L3 comprising the amino acid sequence of GVPDRFSGSGSGTDFTLTISSLQAEDVAVYYC (SEQ ID NO: 9); and an FR-L4 comprising the amino acid sequence of FGPGTKVEIK (SEQ ID NO: 10).

In some instances, the anti-TIGIT antagonist antibody further comprises at least one, two, three, or four of the following heavy chain variable region FRs: an FR-H1 comprising the amino acid sequence of X₁VQLQQSGPGLVKPSQTLSLTCAISGDSVS (SEQ ID NO: 11), wherein X₁ is E or Q; an FR-H2 comprising the amino acid sequence of WIRQSPSRGLEWLG (SEQ ID NO: 12); an FR-H3 comprising the amino acid sequence of RITINPDTSKNQFSLQLNSVTPEDTAVFYCTR (SEQ ID NO: 13); and/or an FR-H4 comprising the amino acid sequence of WGQGTLVTVSS (SEQ ID NO: 14), or a combination of one or more of the above FRs and one or more variants thereof having at least about 90% sequence identity (e.g., 90%, 91%, 92%, 93%, 94%, 95%, 96%, 97%, 98%, or 99% identity) to any one of SEQ ID NOs: 11-14. The anti-TIGIT antagonist antibody may further include, for example, at least one, two, three, or four of the following heavy chain variable region FRs: an FR-H1 comprising the amino acid sequence of EVQLQQSGPGLVKPSQTLSLTCAISGDSVS (SEQ ID NO: 15); an FR-H2 comprising the amino acid sequence of WIRQSPSRGLEWLG (SEQ ID NO: 12); an FR-H3 comprising the amino acid sequence of RITINPDTSKNQFSLQLNSVTPEDTAVFYCTR (SEQ ID NO: 13); and/or an FR-H4 comprising the amino acid sequence of WGQGTLVTVSS (SEQ ID NO: 14), or a combination of one or more of the above FRs and one or more variants thereof having at least about 90% sequence identity (e.g., 90%, 91%, 92%, 93%, 94%, 95%, 96%, 97%, 98%, or 99% identity) to any one of SEQ ID NOs: 12-15. In some instances, the anti-TIGIT antagonist antibody includes an FR-H1 comprising the amino acid sequence of EVQLQQSGPGLVKPSQTLSLTCAISGDSVS (SEQ ID NO: 15); an FR-H2 comprising the amino acid sequence of WIRQSPSRGLEWLG (SEQ ID NO: 12); an FR-H3 comprising the amino acid sequence of RITINPDTSKNQFSLQLNSVTPEDTAVFYCTR (SEQ ID NO: 13); and an FR-H4 comprising the amino acid sequence of WGQGTLVTVSS (SEQ ID NO: 14. In another instance, for example, the anti-TIGIT antagonist antibody may further include at least one, two, three, or four of the following heavy chain variable region FRs: an FR-H1 comprising the amino acid sequence of QVQLQQSGPGLVKPSQTLSLTCAISGDSVS (SEQ ID NO: 16); an FR-H2 comprising the amino acid sequence of WIRQSPSRGLEWLG (SEQ ID NO: 12); an FR-H3 comprising the amino acid sequence of RITINPDTSKNQFSLQLNSVTPEDTAVFYCTR (SEQ ID NO: 13); and/or an FR-H4 comprising the amino acid sequence of WGQGTLVTVSS (SEQ ID NO: 14), or a combination of one or more of the above FRs and one or more variants thereof having at least about 90% sequence identity (e.g., 90%, 91%, 92%, 93%, 94%, 95%, 96%, 97%, 98%, or 99% identity) to any one of SEQ ID NOs: 12-14 and 16. In some instances, the anti-TIGIT antagonist antibody includes an FR-H1 comprising the amino acid sequence of QVQLQQSGPGLVKPSQTLSLTCAISGDSVS (SEQ ID NO: 16); an FR-H2 comprising the amino acid sequence of WIRQSPSRGLEWLG (SEQ ID NO: 12); an FR-H3 comprising the amino acid sequence of RITINPDTSKNQFSLQLNSVTPEDTAVFYCTR (SEQ ID NO: 13); and an FR-H4 comprising the amino acid sequence of WGQGTLVTVSS (SEQ ID NO: 14).

In another aspect, an anti-TIGIT antagonist antibody is provided, wherein the antibody comprises a VH as in any of the instances provided above, and a VL as in any of the instances provided above, wherein one or both of the variable domain sequences include post-translational modifications.

In some instances, any one of the anti-TIGIT antagonist antibodies described above is capable of binding to rabbit TIGIT, in addition to human TIGIT. In some instances, any one of the anti-TIGIT antagonist antibodies described above is capable of binding to both human TIGIT and cynomolgus monkey (cyno) TIGIT. In some instances, any one of the anti-TIGIT antagonist antibodies described above is capable of binding to human TIGIT, cyno TIGIT, and rabbit TIGIT. In some instances, any one of the anti-TIGIT antagonist antibodies described above is capable of binding to human TIGIT, cyno TIGIT, and rabbit TIGIT, but not murine TIGIT.

In some instances, the anti-TIGIT antagonist antibody binds human TIGIT with a K_{D} of about 10 nM or lower and cyno TIGIT with a K_{D} of about 10 nM or lower (e.g., binds human TIGIT with a K_{D} of about 0.1 nM to about 1 nM and cyno TIGIT with a K_{D} of about 0.5 nM to about 1 nM, e.g., binds human TIGIT with a K_{D} of about 0.1 nM or lower and cyno TIGIT with a K_{D} of about 0.5 nM or lower).

In some instances, the anti-TIGIT antagonist antibody specifically binds TIGIT and inhibits or blocks TIGIT interaction with poliovirus receptor (PVR) (e.g., the antagonist antibody inhibits intracellular signaling mediated by TIGIT binding to PVR). In some instances, the antagonist antibody inhibits or blocks binding of human TIGIT to human PVR with an IC50 value of 10 nM or lower (e.g., 1 nM to about 10 nM). In some instances, the anti-TIGIT antagonist antibody specifically binds TIGIT and inhibits or blocks TIGIT interaction with PVR, without impacting PVR-CD226 interaction. In some instances, the antagonist antibody inhibits or blocks binding of cyno TIGIT to cyno PVR with an IC50 value of 50 nM or lower (e.g., 1 nM to about 50 nM, e.g., 1 nM to about 5 nM). In some instances, the anti-TIGIT antagonist antibody inhibits and/or blocks the interaction of CD226 with TIGIT. In some instances, the anti-TIGIT antagonist antibody inhibits and/or blocks the ability of TIGIT to disrupt CD226 homodimerization. In some instances, the methods or uses described herein may include using or administering an isolated anti-TIGIT antagonist antibody that competes for binding to TIGIT with any of the anti-TIGIT antagonist antibodies described above. For example, the method may include administering an isolated anti-TIGIT antagonist antibody that competes for binding to TIGIT with an anti-TIGIT antagonist antibody having the following six HVRs: (a) an HVR-H1 comprising the amino acid sequence of SNSAAWN (SEQ ID NO: 1); (b) an HVR-H2 comprising the amino acid sequence of KTYYRFKWYSDYAVSVKG (SEQ ID NO: 2); (c) an HVR-H3 comprising the amino acid sequence of ESTTYDLLAGPFDY (SEQ ID NO: 3); (d) an HVR-L1 comprising the amino acid sequence of KSSQTVLYSSNNKKYLA (SEQ ID NO: 4), (e) an HVR-L2 comprising the amino acid sequence of WASTRES (SEQ ID NO: 5); and (f) an HVR-L3 comprising the amino acid sequence of QQYYSTPFT (SEQ ID NO: 6). The methods described herein may also include administering an isolated anti-TIGIT antagonist antibody that binds to the same epitope as an anti-TIGIT antagonist antibody described above.

In some aspects, the anti-TIGIT antagonist antibody is an antibody having intact Fc-mediated effector function (e.g., tiragolumab, vibostolimab, etigilimab, EOS084448, or TJ-T6) or enhanced effector function (e.g., SGN-TGT).

In other aspects, the anti-TIGIT antagonist antibody is an antibody that lacks Fc-mediated effector function (e.g., domvanalimab, BMS-986207, ASP8374, or COM902).

In some aspects, the anti-TIGIT antagonist antibody is an IgG1 class antibody, e.g., tiragolumab, vibostolimab, domvanalimab, BMS-986207, etigilimab, BGB-A1217, SGN-TGT, EOS084448 (EOS-448), TJ-T6, or AB308.

In other aspects, the anti-TIGIT antagonist antibody is an IgG4 class antibody, e.g., ASP8374 or COM902.

The anti-TIGIT antagonist antibodies (e.g., tiragolumab) useful in this invention, including compositions containing such antibodies, may be used in combination with a PD-1 axis binding antagonist (e.g., PD-L1 binding antagonists (e.g., anti-PD-L1 antagonist antibodies, e.g., atezolizumab), PD-1 binding antagonists (e.g., anti-PD-1 antagonist antibodies, e.g., pembrolizumab), and PD-L2 binding antagonists (e.g., anti-PD-L2 antagonist antibodies)).

In some embodiments, the anti-TIGIT antagonist antibody functions to inhibit TIGIT signaling. In some embodiments, the anti-TIGIT antagonist antibody inhibits the binding of TIGIT to its binding partners. Exemplary TIGIT binding partners include CD155 (PVR), CD112 (PVRL2 or Nectin-2), and CD113 (PVRL3 or Nectin-3). In some embodiments, the anti-TIGIT antagonist antibody is capable of inhibiting binding between TIGIT and CD155. In some embodiments, the anti-TIGIT antagonist antibody may inhibit binding between TIGIT and CD112. In some embodiments, the anti-TIGIT antagonist antibody inhibits binding between TIGIT and CD113. In some embodiments, the anti-TIGIT antagonist antibody inhibits TIGIT-mediated cellular signaling in immune cells. In some embodiments, the anti-TIGIT antagonist antibody inhibits TIGIT by depleting regulatory T cells (e.g., when engaging a FcγR).

In some embodiments, the anti-TIGIT antibody is a monoclonal antibody. In some embodiments, the anti-TIGIT antibody is an antibody fragment selected from the group consisting of Fab, Fab′-SH, Fv, scFv, and (Fab′)₂ fragments. In some embodiments, the anti-TIGIT antibody is a humanized antibody. In some embodiments, the anti-TIGIT antibody is a human antibody. In some embodiments, the anti-TIGIT antibody described herein binds to human TIGIT. In some embodiments, the anti-TIGIT antibody is an Fc fusion protein.

In some embodiments, the anti-TIGIT antibody is selected from the group consisting of tiragolumab (MTIG7192A, RG6058 or R07092284), vibostolimab (MK-7684), ASP8374 (PTZ-201), EOS884448 (EOS-448), SEA-TGT (SGN-TGT)), BGB-A1217, BMS-986207 (ONO-4686), COM902 (CGEN-15137), IB1939, domvanalimab (AB154), M6223, AB308, AB154, TJ-T6, MG1131, NB6253, HLX301, HLX53, SL-9258 (TIGIT-Fc-LIGHT), STW264, and YBL-012. In some embodiments, the anti-TIGIT antibody is selected from the group consisting of tiragolumab (MTIG7192A, RG6058 or R07092284), vibostolimab (MK-7684), ASP8374 (PTZ-201), EOS-448, and SEA-TGT (SGN-TGT). The anti-TIGIT antibody may be tiragolumab (MTIG7192A, RG6058 or R07092284).

Non-limiting examples of anti-TIGIT antibodies that are useful for the methods disclosed herein, and methods for making thereof are described in PCT Pub. Nos. WO2018183889A1, WO2019129261Ai, WO2016106302A9, WO2018033798A1, WO2020020281Ai, WO2019023504A1, WO2017152088A1, WO2016028656A1, WO2017030823A2, WO2018204405A1, WO2019152574A1, and WO2020041541A2; U.S. Pat. Nos. 10,189,902, 10,213,505, 10,124,061, 10,537,633, and 10,618,958; and U.S. Pub. Nos. 2020/0095324, 2019/0112375, 2018/0371083, and 2020/0062859, each of which is incorporated herein by reference in its entirety. Additional non-limiting examples of anti-TIGIT antibodies, useful for the methods of disclosed herein, and methods for making thereof are described in PCT Pub. Nos. WO2018204363A1, WO2018047139A1, WO2019175799A2, WO2018022946A1, WO2015143343A2, WO2018218056A1, WO2019232484A1, WO2019079777A1, WO2018128939A1, WO2017196867A1, WO201 91 5441 5A1, WO2019062832A1, WO2018234793A3, WO2018102536A1, WO2019137548A1, WO2019129221Ai, WO2018102746A1, WO2018160704A9, WO2020041541A2, WO2019094637A9, WO2017037707A1, WO2019168382A1, WO2006124667A3, WO2017021526A1, WO201 718461 9A2, WO2017048824A1, WO2019032619A9, WO201 81571 62A1, WO2020176718A1, WO2020047329A1, WO2020047329A1, WO2018220446A9; U.S. Pat. Nos. 9,617,338, 9,567,399, 10,604,576, and 9,994,637; and Pub. Nos. US 2018/0355040, US 2019/0175654, US 2019/0040154, US 2019/0382477, US 2019/0010246, US 2020/0164071, US 2020/0131267, US 2019/0338032, US 2019/0330351, US 2019/0202917, US 2019/0284269, US 2018/0155422, US 2020/0040082, US 2019/0263909, US 2018/0185480, US 2019/0375843, US 2017/0037133, US 2019/0077869, US 2019/0367579, US 2020/0222503, US 2020/0283496, CN109734806A, and CN1 10818795A, each of which is incorporated herein by reference in its entirety.

The anti-TIGIT antibodies useful in the methods disclosed herein include ASP8374 (PTZ-201), BGB-A1217, BMS-986207 (ONO-4686), COM902 (CGEN-15137), M6223, IB1939, EOS-448, domvanalimab (AB154), vibostolimab (MK-7684), and SEA-TGT (SGN-TGT). Additional anti-TIGIT antibodies useful in the methods disclosed herein include AGEN1307; AGEN1777; antibody clones pab2197 and pab2196 (Agenus Inc.); antibody clones TBB8, TDC8, 3TB3, 5TB10, and D1Y1A (Anhui Anke Biotechnology Group Co. Ltd.), antibody clones MAB1, MAB2, MAB3, MAB4, MAB5, MAB6, MAB 7, MAB8, MAB9, MAB 10, MAB 11, MAB 12, MAB13, MAB 14, MAB 15, MAB 16, MAB 17, MAB 18, MAB19, MAB20, MAB21 (Astellas Pharma/Potenza Therapeutics), antibody clones hu1217-1-1 and hu1217-2-2 (BeiGene), antibody clones 4D4 and 19G (Brigham & Women's Hospital), antibody clones 11 G11, 10D7, 15A6, 22G2, TIGIT G2a, and TIGIT G1 D265A, including such antibodies with modified heavy chain constant regions (Bristol-Myers Squibb); antibody clones 10A7, CPA.9.086, CPA.9.083.H4(S241 P), CPA.9.086.H4(S241 P), CHA.9.547.7.H4(S241 P) and CHA.9.547.13.H4(S241 P) (Compugen); anti-PVRIG/anti-TIGIT bispecific antibodies (Compugen), antibody clones 315293, 328189, 350426, 326504, and 331672 (Fred Hutchinson Cancer Research Center); antibody clones T-01, T-02, T-03, T-04, T-05, T-06, T-07, T-08, T-09, and T-10 (Gensun BioPharma Inc.); antibody clones 1H6, 2B11, 3A10, 4A5, 4A9, 4H5, 6A2, 6B7, 7F4, 8E1, 8G3, 9F4, 9G6, 10C1, 10F10, 11G4, 12B7, 12C8, 15E9, 16C11, 16D6, and 16E10 (Hefei Ruida Immunological Drugs Research Institute Co. Ltd.); antibody clones h3C5H1, h3C5H2, h3C5H3, h3C5H4, h3C5H3-1, h3C5H3-2, h3C5H3-3, h3C5L1, and h3C5L2 (IGM Biosciences Inc.); antibody clones 90D9, 101 E1, 116H8, 118A12, 131A12, 143B6, 167F7, 221 F11, 222H4, 327C9, 342A9, 344F2, 349H6, and 350D10 (I-Mab Biopharma); antibody clones ADI-27238, ADI-30263, ADI-30267, ADI-30268, ADI-27243, ADI-30302, ADI-30336, ADI-27278, ADI-30193, ADI-30296, ADI-27291, ADI-30283, ADI-30286, ADI-30288, AD127297, ADI-30272, ADI-30278, ADI-27301, ADI-30306, and ADI-30311 (Innovent Biologics, Inc.); antibody clones 26518, 29478, 26452, 29487, 29489, 31282, 26486, 29494, 29499, 26521, 29513, 26493, 29520, 29523, 29527, 31288, 32919, 32931, 26432, and 32959 (iTeos Therapeutics); antibody clones m1707, m1708, m1709, m1710, m1711, h1707, h1708, h1709, h1710, and h1711 (Jiangsu Hengrui Medicine Co. Ltd.); antibody clones TIG1, TIG2, and TIG3 (JN Biosciences LLC); antibody clones (e.g., KY01, KY02, KY03, KY04, KY05, KY06, KY07, KY08, KY09, KY10, K11, K12, K13, K14, K15, K16, K17, K18, K19, K20, K21, K22, K23 Kymab TIGIT (Antibody 2), and Tool TIGIT (Antibody 4) (Kymab Limited); bispecific antibodies 1 D05/in-house anti-TIGIT with 1 D05 (anti-PD-L1) Native variable domain and Kymab TIGIT antigen binding site (ABS) domain (Bispecific 1), In-house anti-TIGIT/1 D05 with Kymab TIGIT Native variable domain and 1 D05 ABS domain (Bispecific 2), Tool anti-TIGIT/Tool anti-PD-L1 with Toon anti-TIGIT Native variable domain and Tool anti-PD-L1 ABS domain (Bispecific 3), Tool anti-PD-L1/Tool anti-TIGIT with Tool anti-PD-L1 Native variable domain and Tool anti-TIGIT ABS domain (Bispecific 4) (Kymab Limited); antibody clones and clone variants 14D7, 26B10, Hu14D7, Hu26B10, 14A6, Hu14A6,28H5,31C6, Hu31C6, 25G10, MBS43, 37D10, 18G10, 11A11, c18G10, and LB155.14A6.G2.A8 (Merck); etigilimab (OMP-313M32) (Mereo BioPharma); antibody clones 64G1 E9B4, 100C4E7D11, 83G5H11C12, 92E9D4B4, 104G12E12G2, 121C2F101B5, 128E3F10F3F2, 70A11A8E6, 11D8E124A, 16F10H12C11, 8F2D8E7, 48B5G4E12,139E2C2D2, 128E3G7F5, AS19584, AS19852, AS19858, AS19886, AS19887, AS19888, AS20160, AS19584VH26, AS19584VH29, AS19584VH30, AS19584VH31, AS19886VH5, AS19886VH8, AS19886VH9, AS19886VH10, AS19886VH19, AS19886VH20, AS19584VH28-Fc, AS19886VH5-Fc, AS19886VH8-Fc, AS19584-Fc, and AS19886-Fc (Nanjing Legend Biotechnology Co. Ltd.); antibody clones ARE clones: Ab58, Ab69, Ab75, Ab133, Ab177, Ab122, Ab86, Ab180, Ab83, Ab26, Ab20, Ab147, Ab12, Ab66, Ab176, Ab96, Ab123, Ab109, Ab149, Ab34, Ab61, Ab64, Ab105, Ab108, Ab178, Ab166, Ab29, Ab135, Ab171, Ab194, Ab184, Ab164, Ab183, Ab158, Ab55, Ab136, Ab39, Ab159, Ab151, Ab139, Ab107, Ab36, Ab193, Ab115, Ab106, Ab13f8, Ab127, Ab165, Ab155, Ab19, Ab6, Ab187, Ab179, Ab65, Ab114, Ab102, Ab94, Ab163, Ab110, Ab80, Ab92, Ab117, Ab162, Ab121, Ab195, Ab84, Ab161, Ab198, Ab24, Ab98, Ab116, Ab174, Ab196, Ab51, Ab91, Ab185, Ab23, Ab7, Ab95, Ab100, Ab140, Ab145, Ab150, Ab168, Ab54, Ab77, Ab43, Ab160, Ab82, Ab189, Ab17, Ab103, Ab18, Ab130, Ab132, Ab134, Ab144; ARG Clones: Ab2, Ab47, Ab49, Ab31, Ab53, Ab40, Ab5, Ab9, Ab48, Ab4, Ab10, Ab37, Ab33, Ab42, Ab45; ARV Clones: Ab44, Ab97, Ab81, Ab188, Ab186, Ab62, Ab57, Ab192, Ab73, Ab60, Ab28, Ab32, Ab78, Ab14, Ab152, Ab72, Ab137, Ab128, Ab169, Ab87, Ab74, Ab172, Ab153, Ab120, Ab13, Ab113, Ab16, Ab56, Ab129, Ab50, Ab90, Ab99, Ab3, Ab148, Ab124, Ab22, Ab41, Ab119, Ab157, Ab27, Ab15, Ab191, Ab190, Ab79, Ab181, Ab146, Ab167, Ab88, Ab199, Ab71, Ab85, Ab59, Ab141, Ab68, Ab143, Ab46, Ab197, Ab175, Ab156, Ab63, Ab11, Ab182, Ab89, Ab8, Ab101, Ab25, Ab154, Ab21, Ab111, Ab118, Ab173, Ab38, Ab76, Ab131, Ab1, Ab67, Ab70, Ab170, Ab30, Ab93, Ab142, Ab104, Ab112, Ab35, Ab126, and Ab125 (Rigel Pharmaceuticals, Inc.); CASC-674 (Seattle Genetics); antibody clones 2, 2C, 3, 5, 13, 13A, 13B, 13C, 13D, 14, 16, 16C, 16D, 16E, 18, 21, 22, 25, 25A, 25B, 25C, 25D, 25E, 27, 54, 13 IgG2a afucosylated, 13 hIgG1 wild-type, and 13 LALA-PG (Seattle Genetics); JS006 (Shanghai Junshi Biosciences Ltd.); anti-TIGIT Fc antibody and bispecific antibody PD1×TIGIT (Xencor), antibody clone VSIG9 #1 (Vsig9.01) and 258-CS1 #4 (#4) (Yissum Research Development Company of The Hebrew University Of Jerusalem Ltd.); YH29143 (Yuhan Co, Ltd.); antibody clones S02, S03, S04, S05, S06, S11, S12, S14, S19, S32, S39, S43, S62, S64, F01, F02, F03, F04, 32D7, 101H3, 10A7, and 1F4 (Yuhan Co, Ltd.); anti-zB7R1 clones 318.4.1.1 (E9310), 318.28.2.1 (E9296), 318.39.1.1 (E9311), 318.59.3.1 (E9400), and 318.77.1.10 (ZymoGenetics, Inc).

In some embodiments, the anti-TIGIT antibody is selected from the group consisting of tiragolumab, ASP8374 (PTZ-201), BGB-A1217, BMS-986207 (ONO-4686), COM902 (CGEN-15137), M6223, IB1939, EOS884448 (EOS-448), domvanalimab (AB154), vibostolimab (MK-7684), and SEA-TGT (SGN-TGT). ASP874 (PTZ-201) is an anti-TIGIT monoclonal antibody described in PCT Pub. No. WO2018183889A1 and US Pub. No. 2020/0095324. BGB-A1217 is an anti-TIGIT antibody as described in PCT Pub. No. WO2019129261A1. BMS-986207 (ONO-4686) is an anti-TIGIT antibody as described in PCT Pub. No. WO2016106302A9, U.S. Pat. No. 10,189,902 and US Pub. No. 2019/0112375. COM902 (CGEN-15137) is an anti-TIGIT antibody as described in PCT Pub. No. WO2018033798A1 and U.S. Pat. Nos. 10,213,505 and 10,124,061. IB1939 is an anti-TIGIT antibody as described in PCT Pub. No. WO2020020281A1. EOS884448 (EOS-448) is an anti-TIGIT antibody described in PCT Pub. No. WO2019023504A1. Domvanalimab (AB154) is an anti-TIGIT monoclonal antibody as described in PCT Pub. No. WO2017152088A1 and U.S. Pat. No. 10,537,633. Vibostolimab (MK-7684) is an anti-TIGIT antibody described in PCT Pub. Nos. WO2016028656A1, WO2017030823A2, WO2018204405A1, and/or WO2019152574A1, U.S. Pat. No. 10,618,958, and US Pub. No. 2018/0371083. SEA-TGT (SGN-TGT) is an anti-TIGIT antibody as described in PCT Pub. No. WO2020041541 A2 and US Pub. No. 2020/0062859.

In some embodiments, the anti-TIGIT antagonist antibody is tiragolumab (CAS Registry Number: 1918185-84-8). Tiragolumab (Genentech) is also known as MTIG7192A, RG6058 or R07092284. Tiragolumab is an anti-TIGIT antagonistic monoclonal antibody described in PCT Pub. No. WO2003072305A8, WO2004024068A3, WO2004024072A3, WO2009126688A2, WO2015009856A2, WO201 6011264A1, WO2016109546A2, WO2017053748A2, and WO2019165434A1, and US Pub. Nos. 2017/0044256, 2017/0037127, 2017/0145093, 2017/260594, 2017/0088613, 2018/0186875, 2019/0119376 and U.S. Pat. No. 9,873,740B2, U.S. Ser. No. 10/626,174B2, U.S. Ser. No. 10/611,836B2, U.S. Pat. No. 9,499,596B2, U.S. Pat. No. 8,431,350B2, U.S. Ser. No. 10/047,158B2, and U.S. Ser. No. 10/017,572B2.

In some embodiments, the anti-TIGIT antibody comprises at least one, two, three, four, five, or six complementarity determining regions (CDRs) of any of the anti-TIGIT antibodies disclosed herein. In some embodiments, the anti-TIGIT antibody comprises the six CDRs of any of the anti-TIGIT antibodies disclosed herein. In some embodiments, the anti-TIGIT antibody comprises the six CDRs of any one of the antibodies selected from the group consisting of tiragolumab, ASP8374 (PTZ-201), BGB-A1217, BMS-986207 (ONO-4686), COM902 (CGEN-15137), M6223, IB1939, EOS884448 (EOS-448), domvanalimab (AB154), vibostolimab (MK-7684), and SEA-TGT (SGN-TGT).

In some embodiments, the anti-TIGIT antibody comprises a heavy chain and a light chain, wherein the heavy chain comprises a heavy chain variable region (VH) sequence of any one of the anti-TIGIT antibodies disclosed herein and the light chain comprises a light chain variable region (VL) of the same antibody. In some embodiments, the anti-TIGIT antibody comprises the VH and VL of an anti-TIGIT antibody selected from the group consisting of tiragolumab, ASP8374 (PTZ-201), BGB-A1217, BMS-986207 (ONO-4686), COM902 (CGEN-15137), M6223, IB1939, EOS884448 (EOS-448), domvanalimab (AB154), vibostolimab (MK-7684), and SEA-TGT (SGN-TGT).

In some embodiments, the anti-TIGIT antibody comprises the heavy chain and the light chain of any of the anti-TIGIT antibodies disclosed herein. In some embodiments, the anti-TIGIT antibody comprises the heavy chain and the light chain of an anti-TIGIT antibody selected from the group consisting of tiragolumab, ASP8374 (PTZ-201), BGB-A1217, BMS-986207 (ONO-4686), COM902 (CGEN-15137), M6223, IB1939, EOS884448 (EOS-448), domvanalimab (AB154), vibostolimab (MK-7684), and SEA-TGT (SGN-TGT).

In some embodiments, an anti-TIGIT antagonist antibody (according to any of the embodiments described herein may incorporate any of the features, singly or in combination, as described in Section C below.

B. PD-1 Axis Binding Antagonists

Provided herein are methods for treating a cancer in a subject (e.g., a human) comprising administering to the subject an effective amount of a PD-1 axis binding antagonist. PD-1 axis binding antagonists may include PD-L1 binding antagonists, PD-1 binding antagonists, and PD-L2 binding antagonists. Any suitable PD-1 axis binding antagonist may be used.

1. PD-L1 Binding Antagonists

In some instances, the PD-L1 binding antagonist inhibits the binding of PD-L1 to one or more of its ligand binding partners. In other instances, the PD-L1 binding antagonist inhibits the binding of PD-L1 to PD-1. In yet other instances, the PD-L1 binding antagonist inhibits the binding of PD-L1 to B7-1. In some instances, the PD-L1 binding antagonist inhibits the binding of PD-L1 to both PD-1 and B7-1. The PD-L1 binding antagonist may be, without limitation, an antibody, an antigen-binding fragment thereof, an immunoadhesin, a fusion protein, an oligopeptide, or a small molecule. In some instances, the PD-L1 binding antagonist is a small molecule that inhibits PD-L1 (e.g., GS-4224, INCB086550, MAX-10181, INCB090244, CA-170, or ABSK041). In some instances, the PD-L1 binding antagonist is a small molecule that inhibits PD-L1 and VISTA. In some instances, the PD-L1 binding antagonist is CA-170 (also known as AUPM-170). In some instances, the PD-L1 binding antagonist is a small molecule that inhibits PD-L1 and TIM3. In some instances, the small molecule is a compound described in WO 2015/033301 and/or WO 2015/033299.

In some instances, the PD-L1 binding antagonist is an anti-PD-L1 antibody. A variety of anti-PD-L1 antibodies are contemplated and described herein. In any of the instances herein, the isolated anti-PD-L1 antibody can bind to a human PD-L1, for example a human PD-L1 as shown in UniProtKB/Swiss-Prot Accession No. Q9NZQ7-1, or a variant thereof. In some instances, the anti-PD-L1 antibody is capable of inhibiting binding between PD-L1 and PD-1 and/or between PD-L1 and B7-1. In some instances, the anti-PD-L1 antibody is a monoclonal antibody. In some instances, the anti-PD-L1 antibody is an antibody fragment selected from the group consisting of Fab, Fab′-SH, Fv, scFv, and (Fab′)₂ fragments. In some instances, the anti-PD-L1 antibody is a humanized antibody. In some instances, the anti-PD-L1 antibody is a human antibody. Exemplary anti-PD-L1 antibodies include atezolizumab, MDX-1105, MEDI4736 (durvalumab), MSB0010718C (avelumab), SHR-1316, CS1001, envafolimab, TQB2450, ZKAB001, LP-002, CX-072, IMC-001, KL-A167, APL-502, cosibelimab, lodapolimab, FAZ053, TG-1501, BGB-A333, BCD-135, AK-106, LDP, GR1405, HLX20, MSB2311, RC98, PDL-GEX, KD036, KY1003, YBL-007, and HS-636. Examples of anti-PD-L1 antibodies useful in the methods of this invention and methods of making them are described in International Patent Application Publication No. WO 2010/077634 and U.S. Pat. No. 8,217,149, each of which is incorporated herein by reference in its entirety.

In some instances, the anti-PD-L1 antibody (e.g., atezolizumab) includes at least one, two, three, four, five, or six HVRs selected from: (a) an HVR-H1 sequence is GFTFSDSWIH (SEQ ID NO: 20); (b) an HVR-H2 sequence is AWISPYGGSTYYADSVKG (SEQ ID NO: 21); (c) an HVR-H3 sequence is RHWPGGFDY (SEQ ID NO: 22), (d) an HVR-L1 sequence is RASQDVSTAVA (SEQ ID NO: 23); (e) an HVR-L2 sequence is SASFLYS (SEQ ID NO: 24); and (f) an HVR-L3 sequence is QQYLYHPAT (SEQ ID NO: 25).

In some instances, the anti-PD-L1 antibody comprises:

(a) an HVR-H1, HVR-H2, and HVR-H3 sequence of SEQ ID NO: 20, SEQ ID NO: 21 and SEQ ID NO: 22, respectively, and

(b) an HVR-L1, HVR-L2, and HVR-L3 sequence of SEQ ID NO: 23, SEQ ID NO: 24 and SEQ ID NO: 25, respectively.

In some instances, the anti-PD-L1 antibody (e.g., atezolizumab) comprises a heavy chain and a light chain sequence, wherein: (a) the heavy chain variable (VH) region sequence comprises the amino acid sequence: EVQLVESGGGLVQPGGSLRLSCAASGFTFSDSWIHWVRQAPGKGLEWVAWISPYGGSTYYADSVKGRF TISADTSKNTAYLQMNSLRAEDTAVYYCARRHWPGGFDYWGQGTLVTVSS (SEQ ID NO: 26); and (b) the light chain variable (VL) region sequence comprises the amino acid sequence:

(SEQ ID NO: 27)
DIQMTQSPSSLSASVGDRVTITCRASQDVSTAVAWYQQKPGKAPKLLI
YSASFLYSGVPSRFSGSGSGTDFTLTISSLQPEDFATYYCQQYLYHPA
TFGQGTKVEIKR.

In some instances, the anti-PD-L1 antibody (e.g., atezolizumab) comprises a heavy chain and a light chain sequence, wherein: (a) the heavy chain comprises the amino acid sequence:

(SEQ ID NO: 28)
EVQLVESGGGLVQPGGSLRLSCAASGFTFSDSWIHWVRQAPGKGLEWV
AWISPYGGSTYYADSVKGRFTISADTSKNTAYLQMNSLRAEDTAVYYC
ARRHWPGGFDYWGQGTLVTVSSASTKGPSVFPLAPSSKSTSGGTAALG
CLVKDYFPEPVTVSWNSGALTSGVHTFPAVLQSSGLYSLSSVVTVPSS
SLGTQTYICNVNHKPSNTKVDKKVEPKSCDKTHTCPPCPAPELLGGPS
VFLFPPKPKDTLMISRTPEVTCVVVDVSHEDPEVKFNWYVDGVEVHNA
KTKPREEQYASTYRVVSVLTVLHQDWLNGKEYKCKVSNKALPAPIEKT
ISKAKGQPREPQVYTLPPSREEMTKNQVSLTCLVKGFYPSDIAVEWES
NGQPENNYKTTPPVLDSDGSFFLYSKLTVDKSRWQQGNVFSCSVMHEA
LHNHYTQKSLSLSPG;

and (b) the light chain comprises the amino acid sequence:

(SEQ ID NO: 29)
DIQMTQSPSSLSASVGDRVTITCRASQDVSTAVAWYQQKPGKAPKLLI
YSASFLYSGVPSRFSGSGSGTDFTLTISSLQPEDFATYYCQQYLYHPA
TFGQGTKVEIKRTVAAPSVFIFPPSDEQLKSGTASVVCLLNNFYPREA
KVQWKVDNALQSGNSQESVTEQDSKDSTYSLSSTLTLSKADYEKHKVY
ACEVTHQGLSSPVTKSFNRGEC.

In some instances, the anti-PD-L1 antibody comprises (a) a VH domain comprising an amino acid sequence comprising having at least 95% sequence identity (e.g., at least 95%, 96%, 97%, 98%, or 99% sequence identity) to, or the sequence of (SEQ ID NO: 26); (b) a VL domain comprising an amino acid sequence comprising having at least 95% sequence identity (e.g., at least 95%, 96%, 97%, 98%, or 99% sequence identity) to, or the sequence of (SEQ ID NO: 27); or (c) a VH domain as in (a) and a VL domain as in (b). In one embodiment, the anti-PD-L1 antibody comprises atezolizumab, which comprises: (a) the heavy chain amino acid sequence of SEQ ID NO: 28, and (b) the light chain amino acid sequence of SEQ ID NO: 29.

In some instances, the anti-PD-L1 antibody is avelumab (CAS Registry Number: 1537032-82-8). Avelumab, also known as MSB0010718C, is a human monoclonal IgG1 anti-PD-L1 antibody (Merck KGaA, Pfizer).

In some instances, the anti-PD-L1 antibody is durvalumab (CAS Registry Number: 1428935-60-7). Durvalumab, also known as MEDI4736, is an Fc-optimized human monoclonal IgG1 kappa anti-PD-L1 antibody (MedImmune, AstraZeneca) described in WO 2011/066389 and US 2013/034559.

In some instances, the anti-PD-L1 antibody is MDX-1105 (Bristol Myers Squibb). MDX-1105, also known as BMS-936559, is an anti-PD-L1 antibody described in WO 2007/005874.

In some instances, the anti-PD-L1 antibody is LY3300054 (Eli Lilly).

In some instances, the anti-PD-L1 antibody is STI-A1014 (Sorrento). STI-A1014 is a human anti-PD-L1 antibody.

In some instances, the anti-PD-L1 antibody is KN035 (Suzhou Alphamab). KN035 is single-domain antibody (dAB) generated from a camel phage display library.

In some instances, the anti-PD-L1 antibody comprises a cleavable moiety or linker that, when cleaved (e.g., by a protease in the tumor microenvironment), activates an antibody antigen binding domain to allow it to bind its antigen, e.g., by removing a non-binding steric moiety. In some instances, the anti-PD-L1 antibody is CX-072 (CytomX Therapeutics).

In some instances, the anti-PD-L1 antibody comprises the six HVR sequences (e.g., the three heavy chain HVRs and the three light chain HVRs) and/or the heavy chain variable domain and light chain variable domain from an anti-PD-L1 antibody described in US 20160108123, WO 2016/000619, WO 2012/145493, U.S. Pat. No. 9,205,148, WO 2013/181634, or WO 2016/061142.

In a still further specific aspect, the anti-PD-L1 antibody has reduced or minimal effector function. In a still further specific aspect, the minimal effector function results from an “effector-less Fc mutation” or aglycosylation mutation. In still a further instance, the effector-less Fc mutation is an N297A or D265A/N297A substitution in the constant region. In still a further instance, the effector-less Fc mutation is an N297A substitution in the constant region. In some instances, the isolated anti-PD-L1 antibody is aglycosylated. Glycosylation of antibodies is typically either N-linked or O-linked. N-linked refers to the attachment of the carbohydrate moiety to the side chain of an asparagine residue. The tripeptide sequences asparagine-X-serine and asparagine-X-threonine, where X is any amino acid except proline, are the recognition sequences for enzymatic attachment of the carbohydrate moiety to the asparagine side chain. Thus, the presence of either of these tripeptide sequences in a polypeptide creates a potential glycosylation site. O-linked glycosylation refers to the attachment of one of the sugars N-acetylgalactosamine, galactose, or xylose to a hydroxyamino acid, most commonly serine or threonine, although 5-hydroxyproline or 5-hydroxylysine may also be used. Removal of glycosylation sites from an antibody is conveniently accomplished by altering the amino acid sequence such that one of the above-described tripeptide sequences (for N-linked glycosylation sites) is removed. The alteration may be made by substitution of an asparagine, serine or threonine residue within the glycosylation site with another amino acid residue (e.g., glycine, alanine, or a conservative substitution).

2. PD-1 Binding Antagonists

In some instances, the PD-1 axis binding antagonist is a PD-1 binding antagonist. For example, in some instances, the PD-1 binding antagonist inhibits the binding of PD-1 to one or more of its ligand binding partners. In some instances, the PD-1 binding antagonist inhibits the binding of PD-1 to PD-L1. In other instances, the PD-1 binding antagonist inhibits the binding of PD-1 to PD-L2. In yet other instances, the PD-1 binding antagonist inhibits the binding of PD-1 to both PD-L1 and PD-L2. The PD-1 binding antagonist may be, without limitation, an antibody, an antigen-binding fragment thereof, an immunoadhesin, a fusion protein, an oligopeptide, or a small molecule. In some instances, the PD-1 binding antagonist is an immunoadhesin (e.g., an immunoadhesin comprising an extracellular or PD-1 binding portion of PD-L1 or PD-L2 fused to a constant region (e.g., an Fc region of an immunoglobulin sequence). For example, in some instances, the PD-1 binding antagonist is an Fc-fusion protein. In some instances, the PD-1 binding antagonist is AMP-224. AMP-224, also known as B7-DCIg, is a PD-L2-Fc fusion soluble receptor described in WO 2010/027827 and WO 2011/066342. In some instances, the PD-1 binding antagonist is a peptide or small molecule compound. In some instances, the PD-1 binding antagonist is AUNP-12 (PierreFabre/Aurigene). See, e.g., WO 2012/168944, WO 2015/036927, WO 2015/044900, WO 2015/033303, WO 2013/144704, WO 2013/132317, and WO 2011/161699. In some instances, the PD-1 binding antagonist is a small molecule that inhibits PD-1.

In some instances, the PD-1 binding antagonist is an anti-PD-1 antibody. A variety of anti-PD-1 antibodies can be utilized in the methods and uses disclosed herein. In any of the instances herein, the PD-1 antibody can bind to a human PD-1 or a variant thereof. In some instances the anti-PD-1 antibody is a monoclonal antibody. In some instances, the anti-PD-1 antibody is an antibody fragment selected from the group consisting of Fab, Fab′, Fab′-SH, Fv, scFv, and (Fab′)₂ fragments. In some instances, the anti-PD-1 antibody is a humanized antibody. In other instances, the anti-PD-1 antibody is a human antibody. Exemplary anti-PD-1 antagonist antibodies include nivolumab, pembrolizumab, MEDI-0680, PDR001 (spartalizumab), REGN2810 (cemiplimab), BGB-108, prolgolimab, camrelizumab, sintilimab, tislelizumab, toripalimab, dostarlimab, retifanlimab, sasanlimab, penpulimab, CS1003, HLX10, SCT-110A, zimberelimab, balstilimab, genolimzumab, BI 754091, cetrelimab, YBL-006, BAT1306, HX008, budigalimab, AMG 404, CX-188, JTX-4014, 609A, Sym021, LZM009, F520, SG001, AM0001, ENUM 244C8, ENUM 388D4, STI-1110, AK-103, and hAb21.

In some instances, the anti-PD-1 antibody is nivolumab (CAS Registry Number: 946414-94-4). Nivolumab (Bristol-Myers Squibb/Ono), also known as MDX-1106-04, MDX-1106, ONO-4538, BMS-936558, and OPDIVO®, is an anti-PD-1 antibody described in WO 2006/121168.

In some instances, the anti-PD-1 antibody is pembrolizumab (CAS Registry Number: 1374853-91-4). Pembrolizumab (Merck), also known as MK-3475, Merck 3475, lambrolizumab, SCH-900475, and KEYTRUDA®, is an anti-PD-1 antibody described in WO 2009/114335.

In some instances, the anti-PD-1 antibody is MEDI-0680 (AMP-514; AstraZeneca). MEDI-0680 is a humanized IgG4 anti-PD-1 antibody.

In some instances, the anti-PD-1 antibody is PDR001 (CAS Registry No. 1859072-53-9; Novartis). PDR001 is a humanized IgG4 anti-PD-1 antibody that blocks the binding of PD-L1 and PD-L2 to PD-1.

In some instances, the anti-PD-1 antibody is REGN2810 (Regeneron). REGN2810 is a human anti-PD-1 antibody.

In some instances, the anti-PD-1 antibody is BGB-108 (BeiGene).

In some instances, the anti-PD-1 antibody is BGB-A317 (BeiGene).

In some instances, the anti-PD-1 antibody is JS-001 (Shanghai Junshi). JS-001 is a humanized anti-PD-1 antibody.

In some instances, the anti-PD-1 antibody is STI-A1110 (Sorrento). STI-A1110 is a human anti-PD-1 antibody.

In some instances, the anti-PD-1 antibody is INCSHR-1210 (Incyte). INCSHR-1210 is a human IgG4 anti-PD-1 antibody.

In some instances, the anti-PD-1 antibody is PF-06801591 (Pfizer).

In some instances, the anti-PD-1 antibody is TSR-042 (also known as ANB011; Tesaro/AnaptysBio).

In some instances, the anti-PD-1 antibody is AM0001 (ARMO Biosciences).

In some instances, the anti-PD-1 antibody is ENUM 244C8 (Enumeral Biomedical Holdings). ENUM 244C8 is an anti-PD-1 antibody that inhibits PD-1 function without blocking binding of PD-L1 to PD-1.

In some instances, the anti-PD-1 antibody is ENUM 388D4 (Enumeral Biomedical Holdings). ENUM 388D4 is an anti-PD-1 antibody that competitively inhibits binding of PD-L1 to PD-1.

In some instances, the anti-PD-1 antibody comprises the six HVR sequences (e.g., the three heavy chain HVRs and the three light chain HVRs) and/or the heavy chain variable domain and light chain variable domain from an anti-PD-1 antibody described in WO 2015/112800, WO 2015/112805, WO 2015/112900, US 20150210769, WO2016/089873, WO 2015/035606, WO 2015/085847, WO 2014/206107, WO 2012/145493, U.S. Pat. No. 9,205,148, WO 2015/119930, WO 2015/119923, WO 2016/032927, WO 2014/179664, WO 2016/106160, and WO 2014/194302.

In a still further specific aspect, the anti-PD-1 antibody has reduced or minimal Fc-mediated effector function. In a still further specific aspect, the minimal Fc-mediated effector function results from an “effector-less Fc mutation” or aglycosylation mutation. In still a further instance, the effector-less Fc mutation is an N297A or D265A/N297A substitution in the constant region. In some instances, the isolated anti-PD-1 antibody is aglycosylated.

3. PD-L2 Binding Antagonists

In some instances, the PD-1 axis binding antagonist is a PD-L2 binding antagonist. In some instances, the PD-L2 binding antagonist is a molecule that inhibits the binding of PD-L2 to its ligand binding partners. In a specific aspect, the PD-L2 binding ligand partner is PD-1. The PD-L2 binding antagonist may be, without limitation, an antibody, an antigen-binding fragment thereof, an immunoadhesin, a fusion protein, an oligopeptide, or a small molecule.

In some instances, the PD-L2 binding antagonist is an anti-PD-L2 antibody. In any of the instances herein, the anti-PD-L2 antibody can bind to a human PD-L2 or a variant thereof. In some instances, the anti-PD-L2 antibody is a monoclonal antibody. In some instances, the anti-PD-L2 antibody is an antibody fragment selected from the group consisting of Fab, Fab′, Fab′-SH, Fv, scFv, and (Fab′)₂ fragments. In some instances, the anti-PD-L2 antibody is a humanized antibody. In other instances, the anti-PD-L2 antibody is a human antibody. In a still further specific aspect, the anti-PD-L2 antibody has reduced or minimal effector function. In a still further specific aspect, the minimal effector function results from an “effector-less Fc mutation” or aglycosylation mutation. In still a further instance, the effector-less Fc mutation is an N297A or D265A/N297A substitution in the constant region. In some instances, the isolated anti-PD-L2 antibody is aglycosylated.

The PD-1 axis binding antagonists (e.g., atezolizumab) useful in this invention, including compositions containing such molecules, may be used in combination with an anti-TIGIT antagonist antibody.

In a further aspect, a PD-1 axis binding antagonist is a PD-1 axis binding antagonist antibody according to any of the above instances may incorporate any of the features, singly or in combination, as described in Section C below.

C. Antibody formats and properties

1. Antibody Affinity

In certain instances, an anti-TIGIT antagonist antibody, PD-1 axis binding antagonist antibody (e.g., anti-PD-L1 antagonist antibody or anti-PD-1 antagonist antibody), anti-VEGF antibody, and/or anti-IL-6R antibody) provided herein has a dissociation constant (K_{D}) of ≤1 μM, ≤100 nM, ≤10 nM, ≤1 nM, ≤0.1 nM, ≤0.01 nM, or ≤0.001 nM (e.g., 10⁻⁸ M or less, e.g., from 10⁻⁸ M to 10⁻¹³ M, e.g., from 10⁻⁹ M to 10⁻¹³ M).

In one instance, K_{D} is measured by a radiolabeled antigen binding assay (RIA). In one instance, an RIA is performed with the Fab version of an antibody of interest and its antigen. For example, solution binding affinity of Fabs for antigen is measured by equilibrating Fab with a minimal concentration of (¹²⁵I)-labeled antigen in the presence of a titration series of unlabeled antigen, then capturing bound antigen with an anti-Fab antibody-coated plate (see, e.g., Chen et al., J. Mol. Biol. 293:865-881 (1999)). To establish conditions for the assay, MICROTITER® multi-well plates (Thermo Scientific) are coated overnight with 5 μg/ml of a capturing anti-Fab antibody (Cappel Labs) in 50 mM sodium carbonate (pH 9.6), and subsequently blocked with 2% (w/v) bovine serum albumin in PBS for two to five hours at room temperature (approximately 23° C.). In a non-adsorbent plate (Nunc #269620), 100 pM or 26 pM [¹²⁵I]-antigen are mixed with serial dilutions of a Fab of interest (e.g., consistent with assessment of the anti-VEGF antibody, Fab-12, in Presta et al., Cancer Res. 57:4593-4599 (1997)). The Fab of interest is then incubated overnight; however, the incubation may continue for a longer period (e.g., about 65 hours) to ensure that equilibrium is reached. Thereafter, the mixtures are transferred to the capture plate for incubation at room temperature (e.g., for one hour). The solution is then removed and the plate washed eight times with 0.1% polysorbate 20 (TWEEN-20®) in PBS. When the plates have dried, 150 μl/well of scintillant (MICROSCINT-20™; Packard) is added, and the plates are counted on a TOPCOUNT™ gamma counter (Packard) for ten minutes. Concentrations of each Fab that give less than or equal to 20% of maximal binding are chosen for use in competitive binding assays.

According to another instance, K_{D} is measured using a BIACORE® surface plasmon resonance assay. For example, an assay using a BIACORE®-2000 or a BIACORE®-3000 (BIAcore, Inc., Piscataway, N.J.) is performed at 25° C. with immobilized antigen CM5 chips at ˜10 response units (RU). In one instance, carboxymethylated dextran biosensor chips (CM5, BIACORE, Inc.) are activated with N-ethyl-N′-(3-dimethylaminopropyl)-carbodiimide hydrochloride (EDC) and N-hydroxysuccinimide (NHS) according to the supplier's instructions. Antigen is diluted with 10 mM sodium acetate, pH 4.8, to 5 μg/ml (˜0.2 μM) before injection at a flow rate of 5 μl/minute to achieve approximately 10 response units (RU) of coupled protein. Following the injection of antigen, 1 M ethanolamine is injected to block unreacted groups. For kinetics measurements, two-fold serial dilutions of Fab (0.78 nM to 500 nM) are injected in PBS with 0.05% polysorbate 20 (TWEEN-20™) surfactant (PBST) at 25° C. at a flow rate of approximately 25 μl/min. Association rates (kₒₙ) and dissociation rates (k_{off}) are calculated using a simple one-to-one Langmuir binding model (BIACORE® Evaluation Software version 3.2) by simultaneously fitting the association and dissociation sensorgrams. The equilibrium dissociation constant (K_{D}) is calculated as the ratio k_{off}/kₒₙ. See, for example, Chen et al., J. Mol. Biol. 293:865-881 (1999). If the on-rate exceeds 10⁶M−¹s−¹ by the surface plasmon resonance assay above, then the on-rate can be determined by using a fluorescent quenching technique that measures the increase or decrease in fluorescence emission intensity (excitation=295 nm; emission=340 nm, 16 nm band-pass) at 25° C. of a 20 nM anti-antigen antibody (Fab form) in PBS, pH 7.2, in the presence of increasing concentrations of antigen as measured in a spectrometer, such as a stop-flow equipped spectrophometer (Aviv Instruments) or a 8000-series SLM-AMINCO™ spectrophotometer (ThermoSpectronic) with a stirred cuvette.

2. Antibody Fragments

In certain instances, an anti-TIGIT antagonist antibody and/or PD-1 axis binding antagonist antibody (e.g., anti-PD-L1 antagonist antibody or anti-PD-1 antagonist antibody) provided herein is an antibody fragment. Antibody fragments include, but are not limited to, Fab, Fab′, Fab′-SH, F(ab′)₂, Fv, and scFv fragments, and other fragments described below. For a review of certain antibody fragments, see Hudson et al. Nat. Med. 9:129-134 (2003). For a review of scFv fragments, see, e.g., Pluckthün, in The Pharmacology of Monoclonal Antibodies, vol. 113, Rosenburg and Moore eds., (Springer-Verlag, New York), pp. 269-315 (1994); see also WO 93/16185; and U.S. Pat. Nos. 5,571,894 and 5,587,458. For discussion of Fab and F(ab′)2 fragments comprising salvage receptor binding epitope residues and having increased in vivo half-life, see U.S. Pat. No. 5,869,046.

Diabodies are antibody fragments with two antigen-binding sites that may be bivalent or bispecific. See, for example, EP 404,097; WO 1993/01161; Hudson et al. Nat. Med. 9:129-134 (2003); and Hollinger et al. Proc. Natl. Acad. Sci. USA 90: 6444-6448 (1993). Triabodies and tetrabodies are also described in Hudson et al. Nat. Med. 9:129-134 (2003).

Single-domain antibodies are antibody fragments comprising all or a portion of the heavy chain variable domain or all or a portion of the light chain variable domain of an antibody. In certain instances, a single-domain antibody is a human single-domain antibody (Domantis, Inc., Waltham, Mass.; see, e.g., U.S. Pat. No. 6,248,516 B1).

Antibody fragments can be made by various techniques, including but not limited to proteolytic digestion of an intact antibody as well as production by recombinant host cells (e.g., E. coli or phage), as described herein. 3. Chimeric and Humanized Antibodies

In certain instances, an anti-TIGIT antagonist antibody and/or PD-1 axis binding antagonist antibody (e.g., anti-PD-L1 antagonist antibody or anti-PD-1 antagonist antibody) provided herein is a chimeric antibody. Certain chimeric antibodies are described, e.g., in U.S. Pat. No. 4,816,567; and Morrison et al. Proc. Natl. Acad. Sci. USA, 81:6851-6855 (1984)). In one example, a chimeric antibody comprises a non-human variable region (e.g., a variable region derived from a mouse, rat, hamster, rabbit, or non-human primate, such as a monkey) and a human constant region. In a further example, a chimeric antibody is a “class switched” antibody in which the class or subclass has been changed from that of the parent antibody. Chimeric antibodies include antigen-binding fragments thereof.

In certain instances, a chimeric antibody is a humanized antibody. Typically, a non-human antibody is humanized to reduce immunogenicity to humans, while retaining the specificity and affinity of the parental non-human antibody. Generally, a humanized antibody comprises one or more variable domains in which HVRs, e.g., CDRs, (or portions thereof) are derived from a non-human antibody, and FRs (or portions thereof) are derived from human antibody sequences. A humanized antibody optionally will also comprise at least a portion of a human constant region. In some instances, some FR residues in a humanized antibody are substituted with corresponding residues from a non-human antibody (e.g., the antibody from which the HVR residues are derived), e.g., to restore or improve antibody specificity or affinity.

Humanized antibodies and methods of making them are reviewed, e.g., in Almagro and Fransson, Front. Biosci. 13:1619-1633 (2008), and are further described, e.g., in Riechmann et al., Nature 332:323-329 (1988); Queen et al., Proc. Nat'l Acad. Sci. USA 86:10029-10033 (1989); U.S. Pat. Nos. 5,821,337, 7,527,791, 6,982,321, and 7,087,409; Kashmiri et al., Methods 36:25-34 (2005) (describing specificity determining region (SDR) grafting); Padlan, Mol. Immunol. 28:489-498 (1991) (describing “resurfacing”); Dall'Acqua et al., Methods 36:43-60 (2005) (describing “FR shuffling”); and Osbourn et al., Methods 36:61-68 (2005) and Klimka et al., Br. J. Cancer, 83:252-260 (2000) (describing the “guided selection” approach to FR shuffling).

Human framework regions that may be used for humanization include but are not limited to: framework regions selected using the “best-fit” method (see, e.g., Sims et al. J. Immunol. 151:2296 (1993)); framework regions derived from the consensus sequence of human antibodies of a particular subgroup of light or heavy chain variable regions (see, e.g., Carter et al. Proc. Natl. Acad. Sci. USA, 89:4285 (1992); and Presta et al. J. Immunol., 151:2623 (1993)); human mature (somatically mutated) framework regions or human germline framework regions (see, e.g., Almagro and Fransson, Front. Biosci. 13:1619-1633 (2008)); and framework regions derived from screening FR libraries (see, e.g., Baca et al., J. Biol. Chem. 272:10678-10684 (1997) and Rosok et al., J. Biol. Chem. 271:22611-22618 (1996)).

4. Human Antibodies

In certain instances, an anti-TIGIT antagonist antibody and/or PD-1 axis binding antagonist antibody (e.g., anti-PD-L1 antagonist antibody or anti-PD-1 antagonist antibody) provided herein is a human antibody. Human antibodies can be produced using various techniques known in the art. Human antibodies are described generally in van Dijk and van de Winkel, Curr. Opin. Pharmacol. 5: 368-74 (2001) and Lonberg, Curr. Opin. Immunol. 20:450-459 (2008).

Human antibodies may be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. In such transgenic mice, the endogenous immunoglobulin loci have generally been inactivated. For review of methods for obtaining human antibodies from transgenic animals, see Lonberg, Nat. Biotech. 23:1117-1125 (2005). See also, e.g., U.S. Pat. Nos. 6,075,181 and 6,150,584 describing XENOMOUSE™ technology; U.S. Pat. No. 5,770,429 describing HUMAB® technology; U.S. Pat. No. 7,041,870 describing K-M MOUSE® technology, and U.S. Patent Application Publication No. US 2007/0061900, describing VELOCIMOUSE® technology). Human variable regions from intact antibodies generated by such animals may be further modified, e.g., by combining with a different human constant region.

Human antibodies can also be made by hybridoma-based methods. Human myeloma and mouse-human heteromyeloma cell lines for the production of human monoclonal antibodies have been described. (See, e.g., Kozbor J. Immunol., 133: 3001 (1984); Brodeur et al., Monoclonal Antibody Production Techniques and Applications, pp. 51-63 (Marcel Dekker, Inc., New York, 1987); and Boerner et al., J. Immunol., 147: 86 (1991).) Human antibodies generated via human B-cell hybridoma technology are also described in Li et al., Proc. Natl. Acad. Sci. USA, 103:3557-3562 (2006). Additional methods include those described, for example, in U.S. Pat. No. 7,189,826 (describing production of monoclonal human IgM antibodies from hybridoma cell lines) and Ni, Xiandai Mianyixue, 26(4):265-268 (2006) (describing human-human hybridomas). Human hybridoma technology (Trioma technology) is also described in Vollmers and Brandlein, Histology and Histopathology, 20(3):927-937 (2005) and Vollmers and Brandlein, Methods and Findings in Experimental and Clinical Pharmacology, 27(3):1 85-91 (2005).

Human antibodies may also be generated by isolating Fv clone variable domain sequences selected from human-derived phage display libraries. Such variable domain sequences may then be combined with a desired human constant domain. Techniques for selecting human antibodies from antibody libraries are described below.

5. Library-Derived Antibodies

Anti-TIGIT antagonist antibody and/or PD-1 axis binding antagonist antibodies (e.g., anti-PD-L1 antagonist antibodies or anti-PD-1 antagonist antibodies) of the invention may be isolated by screening combinatorial libraries for antibodies with the desired activity or activities. For example, a variety of methods are known in the art for generating phage display libraries and screening such libraries for antibodies possessing the desired binding characteristics. Such methods are reviewed, e.g., in Hoogenboom et al. in Methods in Molecular Biology 178:1-37 (O'Brien et al., ed., Human Press, Totowa, N.J., 2001) and further described, e.g., in the McCafferty et al., Nature 348:552-554; Clackson et al., Nature 352: 624-628 (1991); Marks et al., J. Mol. Biol. 222: 581-597 (1992); Marks and Bradbury, in Methods in Molecular Biology 248:161-175 (Lo, ed., Human Press, Totowa, N.J., 2003); Sidhu et al., J. Mol. Biol. 338(2): 299-310 (2004); Lee et al., J. Mol. Biol. 340(5): 1073-1093 (2004); Fellouse, Proc. Natl. Acad. Sci. USA 101(34): 12467-12472 (2004); and Lee et al., J. Immunol. Methods 284(1-2): 119-132 (2004).

In certain phage display methods, repertoires of VH and VL genes are separately cloned by polymerase chain reaction (PCR) and recombined randomly in phage libraries, which can then be screened for antigen-binding phage as described in Winter et al., Ann. Rev. Immunol., 12: 433-455 (1994). Phage typically display antibody fragments, either as single-chain Fv (scFv) fragments or as Fab fragments. Libraries from immunized sources provide high-affinity antibodies to the immunogen without the requirement of constructing hybridomas. Alternatively, the naive repertoire can be cloned (e.g., from human) to provide a single source of antibodies to a wide range of non-self and also self antigens without any immunization as described by Griffiths et al., EMBO J, 12: 725-734 (1993). Finally, naive libraries can also be made synthetically by cloning unrearranged V-gene segments from stem cells, and using PCR primers containing random sequence to encode the highly variable CDR3 regions and to accomplish rearrangement in vitro, as described by Hoogenboom and Winter, J. Mol. Biol., 227: 381-388 (1992). Patent publications describing human antibody phage libraries include, for example: U.S. Pat. No. 5,750,373, and US Patent Publication Nos. 2005/0079574, 2005/0119455, 2005/0266000, 2007/0117126, 2007/0160598, 2007/0237764, 2007/0292936, and 2009/0002360.

Anti-TIGIT antagonist antibody and/or PD-1 axis binding antagonist antibodies (e.g., anti-PD-L1 antagonist antibodies) or antibody fragments isolated from human antibody libraries are considered human antibodies or human antibody fragments herein.

6. Antibody Variants

In certain instances, amino acid sequence variants of the anti-TIGIT antagonist antibodies and/or PD-1 axis binding antagonist antibodies (e.g., anti-PD-L1 antagonist antibodies or anti-PD-1 antagonist antibodies) of the invention are contemplated. As described in detail herein, anti-TIGIT antagonist antibodies and PD-1 axis binding antagonist antibodies (e.g., anti-PD-L1 antagonist antibodies) may be optimized based on desired structural and functional properties. For example, it may be desirable to improve the binding affinity and/or other biological properties of the antibody. Amino acid sequence variants of an antibody may be prepared by introducing appropriate modifications into the nucleotide sequence encoding the antibody, or by peptide synthesis. Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristics, for example, antigen-binding.

I. Substitution, Insertion, and Deletion Variants

In certain instances, anti-TIGIT antagonist antibody and/or PD-1 axis binding antagonist antibody (e.g., anti-PD-L1 antagonist antibody or anti-PD-1 antagonist antibody) variants having one or more amino acid substitutions are provided. Sites of interest for substitutional mutagenesis include the HVRs and FRs. Conservative substitutions are shown in Table 3 under the heading of “preferred substitutions.” More substantial changes are provided in Table 3 under the heading of “exemplary substitutions,” and as further described below in reference to amino acid side chain classes. Amino acid substitutions may be introduced into an antibody of interest and the products screened for a desired activity, for example, retained/improved antigen binding, decreased immunogenicity, or improved ADCC or CDC.

TABLE 3
Exemplary and Preferred Amino Acid Substitutions

| Original | Exemplary | Preferred |
|---|---|---|
| Residue | Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

Amino acids may be grouped according to common side-chain properties:
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.

Non-conservative substitutions will entail exchanging a member of one of these classes for another class.

One type of substitutional variant involves substituting one or more hypervariable region residues of a parent antibody (e.g., a humanized or human antibody). Generally, the resulting variant(s) selected for further study will have modifications (e.g., improvements) in certain biological properties (e.g., increased affinity, reduced immunogenicity) relative to the parent antibody and/or will have substantially retained certain biological properties of the parent antibody. An exemplary substitutional variant is an affinity matured antibody, which may be conveniently generated, e.g., using phage display-based affinity maturation techniques such as those described herein. Briefly, one or more HVR residues are mutated and the variant antibodies displayed on phage and screened for a particular biological activity (e.g., binding affinity).

Alterations (e.g., substitutions) may be made in HVRs, e.g., to improve antibody affinity. Such alterations may be made in HVR “hotspots,” i.e., residues encoded by codons that undergo mutation at high frequency during the somatic maturation process (see, e.g., Chowdhury, Methods Mol. Biol. 207:179-196 (2008)), and/or residues that contact antigen, with the resulting variant VH or VL being tested for binding affinity. Affinity maturation by constructing and reselecting from secondary libraries has been described, e.g., in Hoogenboom et al. in Methods in Molecular Biology 178:1-37 (O'Brien et al., ed., Human Press, Totowa, N.J., (2001)). In some instances of affinity maturation, diversity is introduced into the variable genes chosen for maturation by any of a variety of methods (e.g., error-prone PCR, chain shuffling, or oligonucleotide-directed mutagenesis). A secondary library is then created. The library is then screened to identify any antibody variants with the desired affinity. Another method to introduce diversity involves HVR-directed approaches, in which several HVR residues (e.g., 4-6 residues at a time) are randomized. HVR residues involved in antigen binding may be specifically identified, e.g., using alanine scanning mutagenesis or modeling. CDR-H3 and CDR-L3 in particular are often targeted.

In certain instances, substitutions, insertions, or deletions may occur within one or more HVRs so long as such alterations do not substantially reduce the ability of the antibody to bind antigen. For example, conservative alterations (e.g., conservative substitutions as provided herein) that do not substantially reduce binding affinity may be made in HVRs. Such alterations may, for example, be outside of antigen contacting residues in the HVRs. In certain instances of the variant VH and VL sequences provided above, each HVR either is unaltered, or includes no more than one, two, or three amino acid substitutions.

A useful method for identification of residues or regions of an antibody that may be targeted for mutagenesis is called “alanine scanning mutagenesis” as described by Cunningham and Wells (1989) Science, 244:1081-1085. In this method, a residue or group of target residues (e.g., charged residues such as Arg, Asp, His, Lys, and Glu) are identified and replaced by a neutral or negatively charged amino acid (e.g., alanine or polyalanine) to determine whether the interaction of the antibody with antigen is affected. Further substitutions may be introduced at the amino acid locations demonstrating functional sensitivity to the initial substitutions. Alternatively, or additionally, a crystal structure of an antigen-antibody complex to identify contact points between the antibody and antigen. Such contact residues and neighboring residues may be targeted or eliminated as candidates for substitution. Variants may be screened to determine whether they contain the desired properties.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody molecule include the fusion to the N- or C-terminus of the antibody to an enzyme (e.g., for ADEPT) or a polypeptide which increases the serum half-life of the antibody.

II. Glycosylation variants

In certain instances, anti-TIGIT antagonist antibodies and/or PD-1 axis binding antagonist antibodies (e.g., anti-PD-L1 antagonist antibodies or anti-PD-1 antagonist antibodies) of the invention can be altered to increase or decrease the extent to which the antibody is glycosylated. Addition or deletion of glycosylation sites to anti-TIGIT antagonist antibody and/or PD-1 axis binding antagonist antibody (e.g., anti-PD-L1 antagonist antibody) of the invention may be conveniently accomplished by altering the amino acid sequence such that one or more glycosylation sites is created or removed.

Where the antibody comprises an Fc region, the carbohydrate attached thereto may be altered. Native antibodies produced by mammalian cells typically comprise a branched, biantennary oligosaccharide that is generally attached by an N-linkage to Asn297 of the CH2 domain of the Fc region. See, e.g., Wright et al. TIBTECH 15:26-32 (1997). The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some instances, modifications of the oligosaccharide in an antibody of the invention are made in order to create antibody variants with certain improved properties.

In one instance, anti-TIGIT antagonist antibody and/or PD-1 axis binding antagonist antibody (e.g., anti-PD-L1 antagonist antibody or anti-PD-1 antagonist antibody) variants are provided having a carbohydrate structure that lacks fucose attached (directly or indirectly) to an Fc region. For example, the amount of fucose in such antibody may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn 297 (e. g. complex, hybrid and high mannose structures) as measured by MALDI-TOF mass spectrometry, as described in WO 2008/077546, for example. Asn297 refers to the asparagine residue located at about position 297 in the Fc region (EU numbering of Fc region residues); however, Asn297 may also be located about ±3 amino acids upstream or downstream of position 297, i.e., between positions 294 and 300, due to minor sequence variations in antibodies. Such fucosylation variants may have improved ADCC function. See, e.g., US Patent Publication Nos. US 2003/0157108 (Presta, L.); US 2004/0093621 (Kyowa Hakko Kogyo Co., Ltd). Examples of publications related to “defucosylated” or “fucose-deficient” antibody variants include: US 2003/01 571 08; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO2005/053742; WO2002/031140; Okazaki et al. J. Mol. Biol. 336:1239-1249 (2004); Yamane-Ohnuki et al. Biotech. Bioeng. 87: 614 (2004). Examples of cell lines capable of producing defucosylated antibodies include Lec13 CHO cells deficient in protein fucosylation (Ripka et al. Arch. Biochem. Biophys. 249:533-545 (1986); US Pat Appl No US 2003/0157108 A1, Presta, L; and WO 2004/056312 A1, Adams et al., especially at Example 11), and knockout cell lines, such as alpha-1,6-fucosyltransferase gene, FUT8, knockout CHO cells (see, e.g., Yamane-Ohnuki et al. Biotech. Bioeng. 87: 614 (2004); Kanda, Y. et al., Biotechnol. Bioeng., 94(4):680-688 (2006); and WO2003/085107).

In view of the above, in some instances, the methods of the invention involve administering to the subject in the context of a fractionated, dose-escalation dosing regimen an anti-TIGIT antagonist antibody (e.g., an anti-TIGIT antagonist antibody disclosed herein, e.g., tiragolumab) and/or PD-1 axis binding antagonist antibody (e.g., anti-PD-L1 antagonist antibody (e.g., atezolizumab) or anti-PD-1 antagonist antibody) variant that comprises an aglycosylation site mutation. In some instances, the aglycosylation site mutation reduces effector function of the antibody. In some instances, the aglycosylation site mutation is a substitution mutation. In some instances, the antibody comprises a substitution mutation in the Fc region that reduces effector function. In some instances, the substitution mutation is at amino acid residue N297, L234, L235, and/or D265 (EU numbering). In some instances, the substitution mutation is selected from the group consisting of N297G, N297A, L234A, L235A, D265A, and P329G. In some instances, the substitution mutation is at amino acid residue N297. In a preferred instance, the substitution mutation is N297A.

Anti-TIGIT antagonist antibody and/or PD-1 axis binding antagonist antibody (e.g., anti-PD-L1 antagonist antibody or anti-PD-1 antagonist antibody) variants are further provided with bisected oligosaccharides, for example, in which a biantennary oligosaccharide attached to the Fc region of the antibody is bisected by GlcNAc. Such antibody variants may have reduced fucosylation and/or improved ADCC function. Examples of such antibody variants are described, e.g., in WO 2003/011878 (Jean-Mairet et al.); U.S. Pat. No. 6,602,684 (Umana et al.); and US 2005/0123546 (Umana et al.). Antibody variants with at least one galactose residue in the oligosaccharide attached to the Fc region are also provided. Such antibody variants may have improved CDC function. Such antibody variants are described, e.g., in WO 1997/30087 (Patel et al.); WO 1998/58964 (Raju, S.); and WO 1999/22764 (Raju, S.).

III. Fc region variants

In certain instances, one or more amino acid modifications are introduced into the Fc region of an anti-TIGIT antagonist (e.g., an anti-TIGIT antagonist antibody disclosed herein, e.g., tiragolumab) antibody and/or PD-1 axis binding antagonist antibody (e.g., anti-PD-L1 antagonist antibody (e.g., atezolizumab) or anti-PD-1 antagonist antibody) of the invention, thereby generating an Fc region variant (see e.g., US 2012/0251531). The Fc region variant may comprise a human Fc region sequence (e.g., a human IgG1, IgG2, IgG3 or IgG4 Fc region) comprising an amino acid modification (e.g., a substitution) at one or more amino acid positions.

In certain instances, the invention contemplates an anti-TIGIT antagonist antibody and/or PD-1 axis binding antagonist antibody (e.g., anti-PD-L1 antagonist antibody variant that possesses some but not all effector functions, which make it a desirable candidate for applications in which the half-life of the antibody in vivo is important yet certain effector functions (such as complement and ADCC) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the antibody lacks FcγR binding (hence likely lacking ADCC activity), but retains FcRn binding ability. The primary cells for mediating ADCC, NK cells, express Fc(RIII only, whereas monocytes express Fc(RI, Fc(RII, and Fc(RIII. FcR expression on hematopoietic cells is summarized in Table 3 on page 464 of Ravetch and Kinet, Annu. Rev. Immunol. 9:457-492 (1991). Non-limiting examples of in vitro assays to assess ADCC activity of a molecule of interest is described in U.S. Pat. No. 5,500,362 (see, e.g., Hellstrom, I. et al. Proc. Nat'l Acad. Sci. USA 83:7059-7063 (1986)) and Hellstrom, I et al., Proc. Nat'l Acad. Sci. USA 82:1499-1502 (1985); 5,821,337 (see Bruggemann, M. et al., J. Exp. Med. 166:1351-1361 (1987)). Alternatively, non-radioactive assays methods may be employed (see, for example, ACTI™ non-radioactive cytotoxicity assay for flow cytometry (CellTechnology, Inc. Mountain View, Calif.; and CYTOTOX 96® non-radioactive cytotoxicity assay (Promega, Madison, Wis.). Useful effector cells for such assays include peripheral blood mononuclear cells (PBMC) and Natural Killer (NK) cells. Alternatively, or additionally, ADCC activity of the molecule of interest may be assessed in vivo, e.g., in an animal model such as that disclosed in Clynes et al. Proc. Nat'l Acad. Sci. USA 95:652-656 (1998). C1q binding assays may also be carried out to confirm that the antibody is unable to bind Clq and hence lacks CDC activity. See, e.g., C1q and C3c binding ELISA in WO 2006/029879 and WO 2005/100402. To assess complement activation, a CDC assay may be performed (see, for example, Gazzano-Santoro et al. J. Immunol. Methods 202:163 (1996); Cragg, M. S. et al. Blood. 101:1045-1052 (2003); and Cragg, M. S. and M. J. Glennie Blood. 103:2738-2743 (2004)). FcRn binding and in vivo clearance/half-life determinations can also be performed using methods known in the art (see, e.g., Petkova, S. B. et al. Int'l. Immunol. 18(12):1759-1769 (2006)).

Antibodies with reduced effector function include those with substitution of one or more of Fc region residues 238, 265, 269, 270, 297, 327 and 329 (U.S. Pat. Nos. 6,737,056 and 8,219,149). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. Nos. 7,332,581 and 8,219,149).

In certain instances, the proline at position 329 of a wild-type human Fc region in the antibody is substituted with glycine or arginine or an amino acid residue large enough to destroy the proline sandwich within the Fc/Fc.gamma receptor interface that is formed between the proline 329 of the Fc and tryptophan residues Trp 87 and Trp 110 of FcgRIII (Sondermann et al.: Nature 406, 267-273 (20 Jul. 2000)). In certain instances, the antibody comprises at least one further amino acid substitution. In one instance, the further amino acid substitution is S228P, E233P, L234A, L235A, L235E, N297A, N297D, or P331S, and still in another instance the at least one further amino acid substitution is L234A and L235A of the human IgG1 Fc region or S228P and L235E of the human IgG4 Fc region (see e.g., US 2012/0251531), and still in another instance the at least one further amino acid substitution is L234A and L235A and P329G of the human IgG1 Fc region.

Certain antibody variants with improved or diminished binding to FcRs are described. (See, e.g., U.S. Pat. No. 6,737,056; WO 2004/056312, and Shields et al., J. Biol. Chem. 9(2): 6591-6604 (2001).)

In certain instance, an antibody variant comprises an Fc region with one or more amino acid substitutions which improve ADCC, e.g., substitutions at positions 298, 333, and/or 334 of the Fc region (EU numbering of residues).

In some instances, alterations are made in the Fc region that result in altered (i.e., either improved or diminished) Clq binding and/or Complement Dependent Cytotoxicity (CDC), e.g., as described in U.S. Pat. No. 6,194,551, WO 99/51642, and Idusogie et al. J. Immunol. 164: 4178-4184 (2000).

Antibodies with increased half-lives and improved binding to the neonatal Fc receptor (FcRn), which is responsible for the transfer of maternal IgGs to the fetus (Guyer et al., J. Immunol. 117:587 (1976) and Kim et al., J. Immunol. 24:249 (1994)), are described in US2005/0014934A1 (Hinton et al.). Those antibodies comprise an Fc region with one or more substitutions therein which improve binding of the Fc region to FcRn. Such Fc variants include those with substitutions at one or more of Fc region residues: 238, 256, 265, 272, 286, 303, 305, 307, 311, 312, 317, 340, 356, 360, 362, 376, 378, 380, 382, 413, 424, or 434, e.g., substitution of Fc region residue 434 (U.S. Pat. No. 7,371,826).

See also Duncan & Winter, Nature 322:738-40 (1988); U.S. Pat. Nos. 5,648,260; 5,624,821; and WO 94/29351 concerning other examples of Fc region variants.

In some aspects, the anti-TIGIT antagonist antibody (e.g., an anti-TIGIT antagonist antibody disclosed herein, e.g., tiragolumab) and/or anti-PD-L1 antagonist antibody (e.g., atezolizumab) comprises an Fc region comprising an N297G mutation (EU numbering).

In some instances, the anti-TIGIT antagonist antibody (e.g., an anti-TIGIT antagonist antibody disclosed herein, e.g., tiragolumab) and/or PD-1 axis binding antagonist antibody (e.g., anti-PD-L1 antagonist antibody (e.g., atezolizumab) or anti-PD-1 antagonist antibody) comprises one or more heavy chain constant domains, wherein the one or more heavy chain constant domains are selected from a first CH1 (CH1,) domain, a first CH2 (CH2₁) domain, a first CH3 (CH3₁) domain, a second CH1 (CH1₂) domain, second CH2 (CH2₂) domain, and a second CH3 (CH3₂) domain. In some instances, at least one of the one or more heavy chain constant domains is paired with another heavy chain constant domain. In some instances, the CH3, and CH3₂ domains each comprise a protuberance or cavity, and wherein the protuberance or cavity in the CH3₁ domain is positionable in the cavity or protuberance, respectively, in the CH3₂domain. In some instances, the CH3₁ and CH3₂domains meet at an interface between said protuberance and cavity. In some instances, the CH2₁ and CH2₂ domains each comprise a protuberance or cavity, and wherein the protuberance or cavity in the CH2₁ domain is positionable in the cavity or protuberance, respectively, in the CH2₂ domain. In other instances, the CH2₁ and CH2₂ domains meet at an interface between said protuberance and cavity. In some instances, the anti-TIGIT antagonist antibody (e.g., an anti-TIGIT antagonist antibody disclosed herein, e.g., tiragolumab) and/or anti-PD-L1 antagonist antibody (e.g., atezolizumab) is an IgG1 antibody.

IV. Cysteine Engineered Antibody Variants

In certain instances, it is desirable to create cysteine engineered anti-TIGIT antagonist antibodies and/or PD-1 axis binding antagonist antibodies (e.g., anti-PD-L1 antagonist antibodies or anti-PD-1 antagonist antibodies), e.g., “thioMAbs,” in which one or more residues of an antibody are substituted with cysteine residues. In particular instances, the substituted residues occur at accessible sites of the antibody. By substituting those residues with cysteine, reactive thiol groups are thereby positioned at accessible sites of the antibody and may be used to conjugate the antibody to other moieties, such as drug moieties or linker-drug moieties, to create an immunoconjugate, as described further herein. In certain instances, any one or more of the following residues are substituted with cysteine: V205 (Kabat numbering) of the light chain; A118 (EU numbering) of the heavy chain; and S400 (EU numbering) of the heavy chain Fc region. Cysteine engineered antibodies may be generated as described, for example, in U.S. Pat. No. 7,521,541.

V. Antibody Derivatives

In certain instances, an anti-TIGIT antagonist antibody of the invention (e.g., an anti-TIGIT antagonist antibody (e.g., tiragolumab) or a variant thereof) and/or PD-1 axis binding antagonist antibody (e.g., anti-PD-L1 antagonist antibody of the invention (e.g., atezolizumab or a variant thereof)) provided herein are further modified to contain additional nonproteinaceous moieties that are known in the art and readily available. The moieties suitable for derivatization of the antibody include but are not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1, 3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, propropylene glycol homopolymers, prolypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody may vary, and if more than one polymer are attached, they can be the same or different molecules. In general, the number and/or type of polymers used for derivatization can be determined based on considerations including, but not limited to, the particular properties or functions of the antibody to be improved, whether the antibody derivative will be used in a therapy under defined conditions, etc.

In another instance, conjugates of an antibody and nonproteinaceous moiety that may be selectively heated by exposure to radiation are provided. In one instance, the nonproteinaceous moiety is a carbon nanotube (Kam et al., Proc. Natl. Acad. Sci. USA 102: 11600-11605 (2005)). The radiation may be of any wavelength, and includes, but is not limited to, wavelengths that do not harm ordinary cells, but which heat the nonproteinaceous moiety to a temperature at which cells proximal to the antibody-nonproteinaceous moiety are killed.

Recombinant Production Methods

Anti-TIGIT antagonist antibodies (e.g., an anti-TIGIT antagonist antibody disclosed herein, e.g., tiragolumab) and/or PD-1 axis binding antagonist antibodies (e.g., anti-PD-L1 antagonist antibodies (e.g., atezolizumab) or anti-PD-1 antagonist antibodies) of the invention may be produced using recombinant methods and compositions, for example, as described in U.S. Pat. No. 4,816,567, which is incorporated herein by reference in its entirety.

For recombinant production of an anti-TIGIT antagonist antibody and/or PD-1 axis binding antagonist antibody (e.g., anti-PD-L1 antagonist antibody or anti-PD-1 antagonist antibody), nucleic acid encoding an antibody, is isolated and inserted into one or more vectors for further cloning and/or expression in a host cell. Such nucleic acid may be readily isolated and sequenced using conventional procedures (e.g., by using oligonucleotide probes that are capable of binding specifically to genes encoding the heavy and light chains of the antibody).

Suitable host cells for cloning or expression of antibody-encoding vectors include prokaryotic or eukaryotic cells described herein. For example, antibodies may be produced in bacteria, in particular when glycosylation and Fc effector function are not needed. For expression of antibody fragments and polypeptides in bacteria, see, e.g., U.S. Pat. Nos. 5,648,237, 5,789,199, and 5,840,523. (See also Charlton, Methods in Molecular Biology, Vol. 248 (B. K. C. Lo, ed., Humana Press, Totowa, N.J., 2003), pp. 245-254, describing expression of antibody fragments in E. coli.) After expression, the antibody may be isolated from the bacterial cell paste in a soluble fraction and can be further purified.

In addition to prokaryotes, eukaryotic microbes such as filamentous fungi or yeast are suitable cloning or expression hosts for antibody-encoding vectors, including fungi and yeast strains whose glycosylation pathways have been “humanized,” resulting in the production of an antibody with a partially or fully human glycosylation pattern. See Gerngross, Nat. Biotech. 22:1409-1414 (2004), and Li et al., Nat. Biotech. 24:210-215 (2006).

Suitable host cells for the expression of glycosylated antibody are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells.

Plant cell cultures can also be utilized as hosts. See, e.g., U.S. Pat. Nos. 5,959,177, 6,040,498, 6,420,548, 7,125,978, and 6,417,429 (describing PLANTIBODIES™ technology for producing antibodies in transgenic plants).

Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293 cells as described, e.g., in Graham et al., J. Gen Virol. 36:59 (1977)); baby hamster kidney cells (BHK); mouse sertoli cells (TM4 cells as described, e.g., in Mather, Biol. Reprod. 23:243-251 (1980)); monkey kidney cells (CV1); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TRI cells, as described, e.g., in Mather et al., Annals N.Y. Acad. Sci. 383:44-68 (1982); MRC 5 cells; and FS4 cells. Other useful mammalian host cell lines include Chinese hamster ovary (CHO) cells, including DHFR⁻ CHO cells (Urlaub et al., Proc. Natl. Acad. Sci. USA 77:4216 (1980)); and myeloma cell lines such as Y0, NS0 and Sp2/0. For a review of certain mammalian host cell lines suitable for antibody production, see, e.g., Yazaki and Wu, Methods in Molecular Biology, Vol. 248 (B. K. C. Lo, ed., Humana Press, Totowa, N.J.), pp. 255-268 (2003).

Immunoconjugates

Also provided are immunoconjugates comprising an anti-TIGIT antagonist antibody as disclosed herein, e.g., tiragolumab and/or PD-1 axis binding antagonist (e.g., anti-PD-L1 antagonist antibody (e.g., atezolizumab) or anti-PD-1 antagonist antibody (e.g., pembrolizumab)) conjugated to one or more cytotoxic agents, such as chemotherapeutic agents or drugs, growth inhibitory agents, toxins (e.g., protein toxins, enzymatically active toxins of bacterial, fungal, plant, or animal origin, or fragments thereof), or radioactive isotopes, for use in the methods or uses described herein.

In some instances, an immunoconjugate is an antibody-drug conjugate (ADC) in which an antibody is conjugated to one or more drugs, including but not limited to a maytansinoid (see U.S. Pat. Nos. 5,208,020, 5,416,064 and European Patent EP 0 425 235 B1); an auristatin such as monomethylauristatin drug moieties DE and DF (MMAE and MMAF) (see U.S. Pat. Nos. 5,635,483 and 5,780,588, and 7,498,298); a dolastatin; a calicheamicin or derivative thereof (see U.S. Pat. Nos. 5,712,374, 5,714,586, 5,739,116, 5,767,285, 5,770,701, 5,770,710, 5,773,001, and 5,877,296; Hinman et al., Cancer Res. 53:3336-3342 (1993); and Lode et al., Cancer Res. 58:2925-2928 (1998)); an anthracycline such as daunomycin or doxorubicin (see Kratz et al., Current Med. Chem. 13:477-523 (2006); Jeffrey et al., Bioorganic & Med. Chem. Letters 16:358-362 (2006); Torgov et al., Bioconj. Chem. 16:717-721 (2005); Nagy et al., Proc. Natl. Acad. Sci. USA 97:829-834 (2000); Dubowchik et al., Bioorg. & Med. Chem. Letters 12:1529-1532 (2002); King et al., J. Med. Chem. 45:4336-4343 (2002); and U.S. Pat. No. 6,630,579); methotrexate; vindesine; a taxane such as docetaxel, paclitaxel, larotaxel, tesetaxel, and ortataxel; a trichothecene; and CC1065.

In another instance, an immunoconjugate comprises an anti-TIGIT antagonist antibody as described herein (e.g., tiragolumab) or a PD-1 axis binding antagonist (e.g., an anti-PD-L1 antagonist antibody (e.g., atezolizumab)) conjugated to an enzymatically active toxin or fragment thereof, including but not limited to diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomycin, and the tricothecenes.

In another instance, an immunoconjugate comprises an anti-TIGIT antagonist antibody as described herein (e.g., tiragolumab) and/or a PD-1 axis binding antagonist (e.g., an anti-PD-L1 antagonist antibody) as described herein (e.g., atezolizumab) conjugated to a radioactive atom to form a radioconjugate. A variety of radioactive isotopes are available for the production of radioconjugates. Examples include At²¹¹, I¹³¹, I¹²⁵, Y⁹⁰, Re¹⁸⁶, Re¹⁸⁸, Sm¹⁵³, Bi²¹², P³², Pb²¹² and radioactive isotopes of Lu. When the radioconjugate is used for detection, it may comprise a radioactive atom for scintigraphic studies, for example tc99m or I123, or a spin label for nuclear magnetic resonance (NMR) imaging (also known as magnetic resonance imaging, mri), such as iodine-123 again, iodine-131, indium-111, fluorine-19, carbon-13, nitrogen-15, oxygen-17, gadolinium, manganese or iron.

Conjugates of an antibody and cytotoxic agent may be made using a variety of bifunctional protein coupling agents such as N-succinimidyl-3-(2-pyridyldithio) propionate (SPDP), succinimidyl-4-(N-maleimidomethyl) cyclohexane-1-carboxylate (SMCC), iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis (p-azidobenzoyl) hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). For example, a ricin immunotoxin can be prepared as described in Vitetta et al., Science 238:1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See WO94/11026. The linker may be a “cleavable linker” facilitating release of a cytotoxic drug in the cell. For example, an acid-labile linker, peptidase-sensitive linker, photolabile linker, dimethyl linker, or disulfide-containing linker (Chari et al., Cancer Res. 52:127-131 (1992); U.S. Pat. No. 5,208,020) may be used.

The immunoconjugates or ADCs herein expressly contemplate, but are not limited to such conjugates prepared with cross-linker reagents including, but not limited to, BMPS, EMCS, GMBS, HBVS, LC-SMCC, MBS, MPBH, SBAP, SIA, SIAB, SMCC, SMPB, SMPH, sulfo-EMCS, sulfo-GMBS, sulfo-KMUS, sulfo-MBS, sulfo-SIAB, sulfo-SMCC, and sulfo-SMPB, and SVSB (succinimidyl-(4-vinylsulfone)benzoate) which are commercially available (e.g., from Pierce Biotechnology, Inc., Rockford, Ill., U.S.A).

ADCs not comprising an anti-TIGIT antagonist antibody or a PD-1 axis binding antagonist may also be used in the methods described herein. In some instances, the ADC is enfortumab vedotin or sacituzumab govitecan.

### D. Chemotherapeutic Agents

1. Platinum-Based Chemotherapeutic Agents

Platinum-based chemotherapeutic agents include an organic compound which contains platinum as an integral part of the molecule. Typically platinum-based chemotherapeutic agents are coordination complexes of platinum. agents include, but are not limited to, carboplatin, cisplatin, and oxaliplatin.

Platinum-based chemotherapeutic agents (such as cisplatin, carboplatin, oxaliplatin, and staraplatin) are widely used antitumor drugs that cause crosslinking of DNA as monoadduct, interstrand crosslinks, intrastrand crosslinks or DNA protein crosslinks. Platinum-based chemotherapeutic agents typically act on the adjacent N-7 position of guanine, forming a 1, 2 intrastrand crosslink (Poklar et al. (1996). Proc. Natl. Acad. Sci. U.S.A. 93 (15): 7606-11; Rudd et al. (1995). Cancer Chemother. Pharmacol. 35 (4): 323-6). The resultant crosslinking inhibits DNA repair and/or DNA synthesis in cancer cells.

Carboplatin is an exemplary platinum coordination compound used in the methods described herein. The chemical name for carboplatin is platinum, diammine[I,I-cyclobutanedicarboxylato(2-)-0,0′]-, (SP-4-2), and carboplatin has the following structural formula:

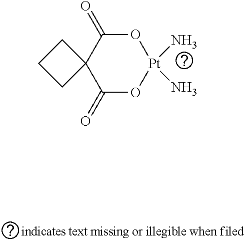

Carboplatin is a crystalline powder with the molecular formula of C6H12N204Pt and a molecular weight of 371.25. It is soluble in water at a rate of approximately 14 mg/mL, and the pH of a 1% solution is 5 to 7. It is virtually insoluble in ethanol, acetone, and dimethylacetamide. Carboplatin produces predominantly interstrand DNA cross-links, and this effect is cell-cycle nonspecific. Carboplatin is commercially available as PARAPLATIN®, BIOCARN, BLASTOCARB, BLASTOPLATIN, CARBOKEM, CARBOMAX, CARBOPA, CARBOPLAN, CARBOTEEN, CARBOTINAL, CYTOCARB, DUCARB, KARPLAT, KEMOCARB, NAPROPLAT, NEOPLATIN, NISCARBO, ONCOCARBIN, TEVACARB, WOMASTIN, and others.

Another exemplary platinum-based chemotherapeutic agent useful in the methods of the present invention is cisplatin, which has the following structure:

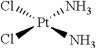

2. Non-Platinum-Based Chemotherapeutic Agents

Non-platinum-based chemotherapeutic agents are another class of chemotherapeutic agents useful as part of the methods, uses, and compositions described herein. Exemplary non-platinum-based chemotherapeutic agents include antimetabolites (e.g., pemetrexed and gemcitabine), topoisomerase II inhibitors (e.g., doxorubicin, etoposide, teniposide, daunorubicin, mitoxantrone, amsacrine, an ellipticine, aurintricarboxylic acid, or HU-331), alkylating agents (e.g., cyclophosphamide), and taxanes (e.g., paclitaxel (e.g., nanoparticle-albumin bound (nab)-paclitaxel), docetaxel, larotaxel, cabazitaxel, milataxel, tesetaxel, and/or orataxel).

3. Antimetabolites

Antimetabolites interfere with and inhibit (wholly or partially) an endogenous (normal) metabolic process within a cell (e.g., a cancer cell). Antimetabolites include gemcitabine, pemetrexed, capecitabine, hydroxyurea, methotrexate, fluorouracil, cladribine, mercaptopurine, and pralatrexate.

Gemcitabine is an exemplary antimetabolite used in the methods described herein and has the following structure:

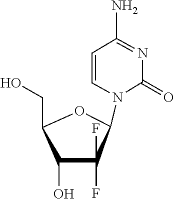

In some instances, pemetrexed can be administered as part of the methods of the present invention. Pemetrexed has the following structure:

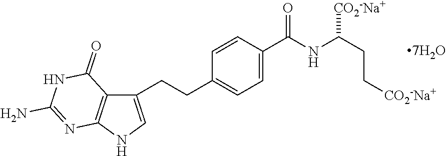

4. Topoisomerase II Inhibitors

Inhibitors of topoisomerase II (e.g., etoposide (VP-16), teniposide, doxorubicin, daunorubicin, mitoxantrone, amsacrine, ellipticines, aurintricarboxylic acid, and HU-331) are also widely used antitumor drugs that stabilize topoisomerase IEDNA covalent complexes (i.e., cleavage complexes) following the formation of enzyme-mediated DNA breaks. The accumulation of such cleavage complexes induces cell death pathways.

Anthracyclines are a type of topoisomerase II inhibitors that are extracted from Streptomyces bacterium. Examples include doxorubicin (adriamycin), daunorubicin (daunomycin), epirubicin, idarubicin, rhodomycin, pyrarubicin, valrubicin, N-trifluoro-acetyl doxorubicin-14-valerate, aclacinomycin, morpholinodoxorubicin (morpholino-DOX), cyanomorpholino-doxorubicin (cyanomorpholino-DOX), 2-pyrrolino-doxorubicin (2-PDOX), 5-iminodaunomycin, mitoxantrone and aclacinomycin A (aclarubicin). In some embodiments, the anthracycline is administered in combination with an alkylating agent, e.g., doxorubicin in combination with cyclophosphamide (treatment with AC).

Doxorubicin (Adriamycin®) is an exemplary topoisomerase II inhibitor used in the methods described herein. It has the chemical name 10-[(3-amino-2,3,6-trideoxy-α-L-lyxo-hexopyrancsyl)oxy]-7,8,9,10-tetrahydro-6,8,11-trihydroxy-8-(2-hydroxyacetyl)-1-methoxy-(8S,10S)-5,12-Naphthacenedione. Doxorubicin has the following structure:

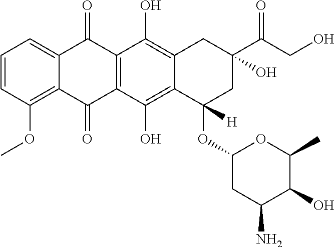 [SMILES: COc1cccc2c1C(=O)c1c(O)c3c(c(O)c1C2=O)C[C@@](O)(C(=O)CO)C[C@@H]3O[C@H]1C[C@H](N)[C@H](O)[C@H](C)O1]

Etoposide is an exemplary topoisomerase II inhibitor used in the methods described herein. Etoposide is typically administered as the prodrug etoposide phosphate, the chemical name for which is: 4′-Demethylepipodophyllotoxin 9-[4,6-0-(R)-ethylidene-Dglucopyranoside], 4′ (dihydrogen phosphate).

Etoposide phosphate has the following structure:

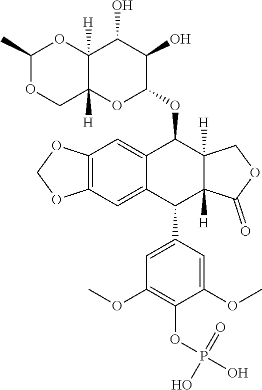

Etoposide phosphate, a phosphate ester of etoposide, is a semi-synthetic derivative of podophyllotoxin and is converted to etoposide by dephosphorylation. Etoposide causes the induction of DNA strand breaks by an interaction with DNA-topoisomerase II or the formation of free radicals, leading to cell cycle arrest (primarily at the G2 stage of the cell cycle) and cell death. Etoposide is commercially available as ETOPOPHOS®, TOPOSAR™, VP-16, VEPESID®, ACTITOP, ASIDE, BIOPOSIDE, CTOP, CYTOP, EPOSED, ESIDE, ETHOPUL, ETOLON, ETONIS, ETOPLAST, ETOSID, ETOVEL, FYTOP, FYTOSID, LASTET, NZYTOP, ONCOSIDE, PLACID, POSID, RETOPSON, TEVASIDE, TOPOK, TOPOSIDE, and others.

5. Taxanes

Taxanes are chemotherapeutic agents that may bind to tubulin, promoting microtubule assembly and stabilization and/or prevent microtubule depolymerization. Taxanes included herein include taxoid 10-deacetylbaccatin III and/or derivatives thereof. Exemplary taxanes include, but are not limited to, paclitaxel (i.e., TAXOL®, CAS #33069-62-4), docetaxel (i.e., TAXOTERE®, CAS #114977-28-5), larotaxel, cabazitaxel, milataxel, tesetaxel, and/or orataxel. In some embodiments, the taxane is an albumin-coated nanoparticle (e.g., nano-albumin bound (nab)-paclitaxel, i.e., ABRAXANE® and/or nab-docetaxel, ABI-008). In some embodiments, the taxane is nab-paclitaxel (ABRAXANE®). In some embodiments, the taxane is formulated in CREMAPHOR® (e.g., TAXOL®) and/or in Tween such as polysorbate 80 (e.g., TAXOTERE®). In some embodiments, the taxane is liposome-encapsulated taxane. In some embodiments, the taxane is a prodrug form and/or conjugated form of taxane (e.g., DHA covalently conjugated to paclitaxel, paclitaxel poliglumex, and/or linoleyl carbonate-paclitaxel). In some embodiments, the paclitaxel is formulated with substantially no surfactant (e.g., in the absence of CREMAPHOR and/or Tween-such as TOCOSOL® paclitaxel).

In some instances, paclitaxel is administered as part of the methods of the present invention. Paclitaxel may have the following structure:

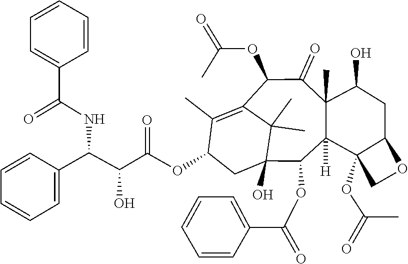 [SMILES: CC(=O)O[C@H]1C(=O)[C@@]2(C)[C@H]([C@H](OC(=O)c3ccccc3)[C@]3(O)C[C@H](OC(=O)[C@H](O)[C@@H](NC(=O)c4ccccc4)c4ccccc4)C(C)=C1C3(C)C)[C@]1(OC(C)=O)CO[C@@H]1C[C@@H]2O]

In some instances, the methods or uses include administration of nano-albumin bound (nab)-paclitaxel.

6. Alkylating Agents

Alkylating agents are chemotherapeutic agents that cause DNA damage. Alkylating agents included nitrogen mustards, nitrosoureas, and alkyl sulfonates. Exemplary alkylating agents include, but are not limited to, cyclophosphamide, 1,3-Bis(2-chloroethyl)-1-nitrosourea, 1,4-butanediol dimethanesulfonate, (2S)-2-amino-3-{4-[bis(2-chloroethyl)amino]phenyl}propanoic acid, 3-(2-chloroethyl)-2-[(2-chloroethyl)amino]tetrahydro-2H-1,3,2-oxazaphosphorine 2-oxide, and 1-(2-chloroethyl)-3-cyclohexyl-1-nitrosourea. In some embodiments, the alkylating agent is cyclophosphamide. Cyclophosphamide may have the following structure:

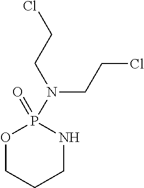 [SMILES: O=P1(N(CCCl)CCCl)NCCCO1]

A skilled artisan will appreciate that any of the aforementioned chemotherapeutic agents can be administered in various forms, such as salt forms, which are contemplated as part of the present invention.

### E. Exemplary Colony Stimulating Factors

Colony stimulating factors are proteins that stimulate the proliferation of cells. Colony stimulating factors include G-CSF (e.g., pegfilgrastim or filgrastim) and GM-CSF (e.g., sargramostim). In some embodiments, the colony stimulating factor is G-CSF (e.g., pegfilgrastim or filgrastim). In some embodiments, the G-CSF is pegfilgrastim. In some embodiments, the G-CSF is filgrastim. In some embodiments, the colony stimulating factor is GM-CSF (e.g., sargramostim). In some embodiments, the GM-CSF is sargramostim.

### F. Exemplary VEGF Antagonists

The invention provides VEGF antagonists useful for treating cancer in a subject (e.g., a human). VEGF antagonists of the invention include any molecule capable of binding VEGF, reducing VEGF expression levels, or neutralizing, blocking, inhibiting, abrogating, reducing, or interfering with VEGF biological activities. An exemplary human VEGF is shown under UniProtKB/Swiss-Prot Accession No. P15692, Gene ID (NCBI): 7422.

In some instances, the VEGF antagonist is an anti-VEGF antibody. In some embodiments, the anti-VEGF antibody is bevacizumab, also known as “rhuMab VEGF” or “AVASTIN®.” Bevacizumab is a recombinant humanized anti-VEGF monoclonal antibody generated according to Presta et al. (Cancer Res. 57:4593-4599, 1997). It comprises mutated human IgG1 framework regions and antigen-binding complementarity-determining regions from the murine anti-hVEGF monoclonal antibody A.4.6.1 that blocks binding of human VEGF to its receptors. Approximately 93% of the amino acid sequence of bevacizumab, including most of the framework regions, is derived from human IgG1, and about 7% of the sequence is derived from the murine antibody A4.6.1. Bevacizumab has a molecular mass of about 149,000 Daltons and is glycosylated. Bevacizumab and other humanized anti-VEGF antibodies are further described in U.S. Pat. No. 6,884,879 issued Feb. 26, 2005, the entire disclosure of which is expressly incorporated herein by reference. In some instances, the anti-VEGF antibody comprises the heavy chain variable (VH) region sequence and the light chain variable (VL) region sequence of bevacizumab.

Additional preferred antibodies include the G6 or B20 series antibodies (e.g., G6-31, B20-4.1), as described in PCT Application Publication No. WO 2005/012359. For additional preferred antibodies see U.S. Pat. Nos. 7,060,269, 6,582,959, 6,703,020; 6,054,297; WO98/45332; WO 96/30046; WO94/10202; EP 0666868B1; U.S. Patent Application Publication Nos. 2006009360, 20050186208, 20030206899, 20030190317, 20030203409, and 20050112126; and Popkov et al. (Journal of Immunological Methods 288:149-164, 2004). Other preferred antibodies include those that bind to a functional epitope on human VEGF comprising of residues F17, M18, D19, Y21, Y25, Q89, 191, K101, E103, and C104, or, alternatively, comprising residues F17, Y21, Q22, Y25, D63, 183, and Q89.

In other instances, the VEGF antagonist is an anti-VEGFR2 antibody or related molecule (e.g., ramucirumab, tanibirumab, aflibercept); an anti-VEGFR1 antibody or related molecules (e.g., icrucumab, aflibercept (VEGF Trap-Eye; EYLEA®), or ziv-aflibercept (VEGF Trap; ZALTRAP®)); a bispecific VEGF antibody (e.g., MP-0250, vanucizumab (VEGF-ANG2), or bispecific antibodies disclosed in US 2001/0236388); a bispecific antibody including a combination of two of anti-VEGF, anti-VEGFR1, and anti-VEGFR2 arms; an anti-VEGFA antibody (e.g., bevacizumab, sevacizumab); an anti-VEGFB antibody; an anti-VEGFC antibody (e.g., VGX-100), an anti-VEGFD antibody; or a nonpeptide small molecule VEGF antagonist (e.g., pazopanib, axitinib, vandetanib, stivarga, cabozantinib, lenvatinib, nintedanib, orantinib, telatinib, dovitinig, cediranib, motesanib, sulfatinib, apatinib, foretinib, famitinib, or tivozanib).

In a further aspect, a VEGF antagonist (e.g., anti-VEGF antibodies (e.g., bevacizumab)) according to any of the above instances may incorporate any of the features, singly or in combination, as described in Section C, above.

G. Additional Therapeutic Agents

Additional therapeutic agents that may be used in the invention (e.g., used in combination with an anti-TIGIT antagonist antibody) include therapeutic antibodies such as alemtuzumab (Campath), bevacizumab (AVASTIN®, Genentech); cetuximab (ERBITUX®, Imclone); panitumumab (VECTIBIX®, Amgen), rituximab (RITUXAN®, Genentech/Biogen Idec), pertuzumab (OMNITARG®, 2C4, Genentech), trastuzumab (HERCEPTIN®, Genentech), tositumomab (Bexxar, Corixia), and the antibody drug conjugate, gemtuzumab ozogamicin (MYLOTARG®, Wyeth). Additional humanized monoclonal antibodies with therapeutic potential as agents in combination with the compounds described include: apolizumab, aselizumab, atlizumab, bapineuzumab, bivatuzumab mertansine, cantuzumab mertansine, cedelizumab, certolizumab pegol, cidfusituzumab, cidtuzumab, daclizumab, eculizumab, efalizumab, epratuzumab, erlizumab, felvizumab, fontolizumab, gemtuzumab ozogamicin, inotuzumab ozogamicin, ipilimumab, labetuzumab, lintuzumab, matuzumab, mepolizumab, motavizumab, motovizumab, natalizumab, nimotuzumab, nolovizumab, numavizumab, ocrelizumab, omalizumab, palivizumab, pascolizumab, pecfusituzumab, pectuzumab, pexelizumab, ralivizumab, ranibizumab, reslivizumab, reslizumab, resyvizumab, rovelizumab, ruplizumab, sibrotuzumab, siplizumab, sontuzumab, tacatuzumab tetraxetan, tadocizumab, talizumab, tefibazumab, tocilizumab, toralizumab, tucotuzumab celmoleukin, tucusituzumab, umavizumab, urtoxazumab, ustekinumab, visilizumab, and the anti-interleukin-12 (ABT-874/J695, Wyeth Research and Abbott Laboratories) which is a recombinant exclusively human-sequence, full-length IgG1 λ antibody genetically modified to recognize interleukin-12 p40 protein.

Therapeutic antibodies also include antibodies that bind to EGFR. Examples of antibodies that bind to EGFR include MAb 579 (ATCC CRL HB 8506), MAb 455 (ATCC CRL HB8507), Mab 225 (ATCC CRL 8508), MAb 528 (ATCC CRL 8509) (see, U.S. Pat. No. 4,943,533, Mendelsohn et al.) and variants thereof, such as chimerized 225 (C225 or Cetuximab; ERBUTIX®) and reshaped human 225 (H225) (see, WO 96/40210, Imclone Systems Inc.); IMC-11F8, a fully human, EGFR-targeted antibody (Imclone); antibodies that bind type II mutant EGFR (U.S. Pat. No. 5,212,290); humanized and chimeric antibodies that bind EGFR as described in U.S. Pat. No. 5,891,996; and human antibodies that bind EGFR, such as ABX-EGF or Panitumumab (see WO98/50433, Abgenix/Amgen); EMD 55900 (Stragliotto et al. EurCancer 32A:636-640 (1996)); EMD7200 (matuzumab) a humanized EGFR antibody directed against EGFR that competes with both EGF and TGF-alpha for EGFR binding (EMD/Merck); human EGFR antibody, HuMax-EGFR (GenMab); fully human antibodies known as E1.1, E2.4, E2.5, E6.2, E6.4, E2.11, E6. 3 and E7.6. 3 and described in U.S. Pat. No. 6,235,883; MDX-447 (Medarex Inc); and mAb 806 or humanized mAb 806 (Johns et al., J. Biol. Chem. 279(29):30375-30384 (2004)). The anti-EGFR antibody may be conjugated with a cytotoxic agent, thus generating an immunoconjugate (see, e.g., EP659,439A2, Merck Patent GmbH).

Additional therapeutic agents that may be used in the methods, compositions, and uses provided herein include agents targeting co-inhibitory targets (e.g., CTLA-4, LAG3, TIM3, BTLA, and/or VISTA), such as a CTLA-4 antagonist, e.g., an anti-CTLA-4 antagonist antibody (e.g., ipilimumab (YERVOY®)); agents targeting co-stimulatory targets (e.g., CD226, OX-40, CD28, CD27, CD137, HVEM, and/or GITR); radiation therapies; anti-angiogenesis agents; apoptotic agents; and anti-tubulin agents.

### V. Pharmaceutical Compositions, Formulations, and Kits

Any of the anti-TIGIT antagonist antibodies, PD-1 axis binding antagonists (e.g., anti-PD-L1 antagonist antibodies or anti-PD-1 antagonist antibodies), VEGF antagonists, chemotherapeutic agents (e.g., platinum-based chemotherapeutic agents (e.g., carboplatin or cisplatin) and non-platinum-based chemotherapeutic agents (e.g., an alkylating agent (e.g., cyclophosphamide), a taxane (e.g., paclitaxel, e.g., nab-paclitaxel), and/or a topoisomerase II inhibitor (e.g., doxorubicin))), ADCs (e.g., enfortumab vedotin or sacituzumab govitecan), and/or CSFs (e.g., pegfilgrastim, filgrastim, or sargramostim) described herein can be used in pharmaceutical compositions and formulations. Pharmaceutical compositions and formulations of an anti-TIGIT antagonist antibody, a PD-1 axis binding antagonist (e.g., an anti-PD-L1 antagonist antibody), a VEGF antagonist, one or more chemotherapeutic agents, an ADC (e.g., enfortumab vedotin or sacituzumab govitecan), and/or a CSF (e.g., pegfilgrastim, filgrastim, or sargramostim) can be prepared by mixing one, two, three, four, or more than four agents having the desired degree of purity with one or more optional pharmaceutically acceptable carriers (Remington's Pharmaceutical Sciences 16th edition, Osol, A. Ed. (1980)), in the form of lyophilized formulations or aqueous solutions. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as polyethylene glycol (PEG). Exemplary pharmaceutically acceptable carriers herein further include insterstitial drug dispersion agents such as soluble neutral-active hyaluronidase glycoproteins (sHASEGP), for example, human soluble PH-20 hyaluronidase glycoproteins, such as rHuPH20 (HYLENEX®, Baxter International, Inc.). Certain exemplary sHASEGPs and methods of use, including rHuPH20, are described in US Patent Publication Nos. 2005/0260186 and 2006/0104968. In one aspect, a sHASEGP is combined with one or more additional glycosaminoglycanases such as chondroitinases.

Exemplary lyophilized antibody formulations are described in U.S. Pat. No. 6,267,958. Aqueous antibody formulations include those described in U.S. Pat. No. 6,171,586 and WO 2006/044908, the latter formulations including a histidine-acetate buffer.

An exemplary atezolizumab formulation comprises glacial acetic acid, L-histidine, polysorbate 20, and sucrose, with a pH of 5.8. For example, atezolizumab may be provided in a 20 mL vial containing 1200 mg of atezolizumab that is formulated in glacial acetic acid (16.5 mg), L-histidine (62 mg), polysorbate 20 (8 mg), and sucrose (821.6 mg), with a pH of 5.8. In another example, atezolizumab may be provided in a 14 mL vial containing 840 mg of atezolizumab that is formulated in glacial acetic acid (11.5 mg), L-histidine (43.4 mg), polysorbate 20 (5.6 mg), and sucrose (575.1 mg) with a pH of 5.8.

An exemplary tiragolumab formulation comprises a histidine solution containing polysorbate 20, sucrose, L-methionine, and WFI. Tiragolumab may be provided in a 15-mL vial containing 10 mL of tiragolumab drug product at an approximate concentration of tiragolumab antibody of 60 mg/mL.

The formulation herein may also contain more than one active ingredients as necessary for the particular indication being treated, preferably those with complementary activities that do not adversely affect each other. For example, it may be desirable to further provide an additional therapeutic agent. Such active ingredients are suitably present in combination in amounts that are effective for the purpose intended.

Active ingredients may be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

Sustained-release preparations may be prepared. Suitable examples of sustained-release preparations include semipermeable matrices of solid hydrophobic polymers containing the antibody, which matrices are in the form of shaped articles, for example, films, or microcapsules. The formulations to be used for in vivo administration are generally sterile. Sterility may be readily accomplished, e.g., by filtration through sterile filtration membranes.

The invention provides kits that include, e.g., an anti-TIGIT antagonist antibody for use in combination with a PD-1 axis binding antagonist for treating a subject having a cancer according to any of the methods described herein.

In another embodiment of the invention, a kit is provided comprising an anti-TIGIT antagonist antibody for use in combination with a PD-1 axis binding antagonist for treating a subject having a cancer according to any of the methods described herein. In some instances, the kit further comprises the PD-1 axis binding antagonist. In some instances, the article of manufacture or kit further comprises package insert comprising instructions for using the anti-TIGIT antagonist antibody in combination with the PD-1 axis binding antagonist to treat or delay progression of a cancer (e.g., in a patient.

In another embodiment of the invention, a kit is provided comprising a PD-1 axis binding antagonist for use in combination with an anti-TIGIT antagonist antibody for treating a subject having a cancer according to any of the methods described herein. In some instances, the kit further comprises the anti-TIGIT antagonist antibody. In some instances, the article of manufacture or kit further comprises package insert comprising instructions for using the PD-1 axis binding antagonist in combination with anti-TIGIT antagonist antibody (e.g., tiragolumab) to treat or delay progression of a cancer in a patient. In another embodiment, a kit comprises tiragolumab for use in combination with atezolizumab for treating a subject having a cancer according to any of the methods described herein. In some embodiments, the kit further comprises atezolizumab. In some instances, the article of manufacture or kit further comprises package insert comprising instructions for using tiragolumab in combination with atezolizumab to treat or delay progression of a cancer in a patient.

In another embodiment, a kit comprises atezolizumab for use in combination with tiragolumab for treating a subject having a cancer according to any of the methods described herein. In some embodiments, the kit further comprises tiragolumab. In some instances, the article of manufacture or kit further comprises package insert comprising instructions for using atezolizumab in combination with tiragolumab to treat or delay progression of cancer in a patient.

In some instances, the PD-1 axis binding antagonist and the anti-TIGIT antagonist antibody are in the same container or separate containers. Suitable containers include, for example, bottles, vials, bags and syringes. The container may be formed from a variety of materials such as glass, plastic (such as polyvinyl chloride or polyolefin), or metal alloy (such as stainless steel or hastelloy). In some instances, the container holds the formulation and the label on, or associated with, the container may indicate directions for use. The article of manufacture or kit may further include other materials desirable from a commercial and user standpoint, including other buffers, diluents, filters, needles, syringes, and package inserts with instructions for use. In some instances, the article of manufacture further includes one or more of another agent (e.g., an additional chemotherapeutic agent or anti-neoplastic agent). Suitable containers for the one or more agent include, for example, bottles, vials, bags and syringes.

In some embodiments, the kit comprising the PD-1 axis binding antagonist and the anti-TIGIT antagonist antibody further comprises a VEGF antagonist, a chemotherapeutic agent (e.g., a platinum-based chemotherapeutic agent or non-platinum-based chemotherapeutic agent), an ADC (e.g., enfortumab vedotin or sacituzumab govitecan), and/or a CSF (e.g., pegfilgrastim, filgrastim, or sargramostim).

Any of the PD-1 axis binding antagonists and/or anti-TIGIT antagonist antibodies described herein may be included in the article of manufacture or kits. Any of the articles of manufacture or kits may include instructions to administer a PD-1 axis binding antagonist and/or anti-TIGIT antagonist antibody to a subject in accordance with any of the methods described herein.

### VI. EXAMPLES

TABLE 4
Table of contents for Examples

| | Title |
|---|---|
| Example 1 | A Phase Ia/Ib, open-label, dose-escalation study of the |
| | safety and pharmacokinetics of tiragolumab as a single |
| | agent and in combination with atezolizumab administered |
| | with and without chemotherapy in patients with locally |
| | advanced or metastatic tumors |
| Example 2 | A Phase Ib/II, open-label, randomized study of atezolizumab |
| | and chemotherapy in combination with tiragolumab |
| Example 3 | A Phase Ib, open-label, multicohort study of the safety, |
| | efficacy, and pharmacokinetics of tiragolumab in |
| | combination with atezolizumab and chemotherapy in |
| | patients with triple-negative breast cancer |
| Example 4 | A Phase Ia/Ib open-label, dose-escalation study of the |
| | safety and pharmacokinetics of tiragolumab as a single |
| | agent and in combination with atezolizumab and/or other |
| | anti-cancer therapies in patients with locally advanced or |
| | metastatic tumors |
| Example 5 | Example 5. Pharmacokinetics, pharmacodynamics, safety, |
| | and efficacy in a phase Ia/Ib dose-escalation study of the |
| | anti-TIGIT antibody tiragolumab as a single agent and |
| | in combination with atezolizumab in patients with |
| | advanced solid tumors |
| Example 6 | Atezolizumab combined with tiragolumab and bevacizumab |
| | for advanced liver cancer |
| Example 7 | A Phase III, randomized, double blind, placebo-controlled |
| | study of atezolizumab plus carboplatin and etoposide with |
| | or without tiragolumab in patients with untreated |
| | extensive-stage small cell lung cancer |
| Example 8 | A Phase II, randomized, double-blind, placebo-controlled |
| | study of tiragolumab in combination with atezolizumab |
| | plus pemetrexed and carboplatin/cisplatin versus |
| | pembrolizumab plus pemetrexed and carboplatin/cisplatin |
| | in patients with previously untreated advanced non- |
| | squamous non-small cell lung cancer |
| Example 9 | A Phase II, open-label, multicenter study evaluating the |
| | safety and efficacy of neoadjuvant and adjuvant tiragolumab |
| | plus atezolizumab, with or without platinum-based |
| | chemotherapy, in patients with previously untreated locally |
| | advanced resectable stage II, IIIA, or select IIIB non- |
| | small cell lung cancer |
| Example 10 | Materials and methods for GO42501 study |
| Example 11 | Assessment of safety for GO42501 study |
| Example 12 | Statistical considerations and analysis plan for GO42501 |
| | study |
| Example 13 | Efficacy of an anti-TIGIT antagonist antibody in |
| | combination with a PD-1 axis binding antagonist in |
| | patients with cervical cancer |
| Example 14 | A Phase Ib, open-label, multicohort study of the safety, |
| | efficacy, and pharmacokinetics of tiragolumab in |
| | combination with atezolizumab and chemotherapy |
| | in patients with triple-negative breast cancer |
| Example 15 | A Phase II, randomized, double-blind study of atezolizumab |
| | plus tiragolumab and atezolizumab plus placebo as first- |
| | line treatment in patients with recurrent/metastatic PD-L1- |
| | positive squamous cell carcinoma of the head and neck |
| Example 16 | A Phase Ib/II, open-label, multicenter, randomized umbrella |
| | study evaluating the efficacy and safety of multiple |
| | immunotherapy-based treatments and combinations in |
| | patients with muscle-invasive bladder cancer (MIBC) |
| Example 17 | Materials and methods for WO39613 study |
| Example 18 | Assessment of safety for WO39613 study |
| Example 19 | Statistical considerations and analysis plan for WO39613 |
| | study |
| Example 20 | A Phase Ib/II, open-label, multicenter, randomized umbrella |
| | study evaluating the efficacy and safety of tiragolumab in |
| | combination with atezolizumab in patients with metastatic |
| | urothelial carcinoma (mUC) |
| Example 21 | Materials and methods for WO39613 mUC study |
| Example 22 | Statistical considerations and analysis plan for WO39613 |
| | mUC study |
| Example 23 | A Phase Ib/II, open-label, multicenter, randomized, umbrella |
| | study evaluating the efficacy and safety of immunotherapy- |
| | based treatment combinations in patients with locally |
| | advanced or metastatic esophageal cancer |
| Example 24 | Materials and methods for YO39609 study |
| Example 25 | Statistical considerations and analysis plan for Y039609 |
| | study |
| Example 26 | PD-L1 as a predictive biomarker for tiragolumab + |
| | atezolizumab treatment |

The following are examples of the methods of the invention. It is understood that various other embodiments may be practiced, given the general description provided above.

### Example 1. A Phase Ia/Ib, Open-Label, Dose-Escalation Study of the Safety and Pharmacokinetics of Tiragolumab as a Single Agent and in Combination with Atezolizumab Administered with and without Chemotherapy in Patients with Locally Advanced or Metastatic Tumors

This study evaluates the safety, PK, pharmacodynamics, and preliminary anti-tumor activity of tiragolumab (MTIG7192A) when administered as a single agent (Phase Ia) or in combination with atezolizumab with and without chemotherapy (Phase Ib) in patients with locally advanced or metastatic tumors. Specific objectives and corresponding endpoints for the study are outlined in Table 5.

TABLE 5
Objectives and Endpoints

| Objectives | Corresponding Endpoints |
|---|---|
| Safety Objective: | |
| To evaluate the safety and tolerability of | Incidence and nature of DLTs |
| tiragolumab when administered as a single agent | Incidence, nature, and severity of adverse events |
| (Phase Ia) or in combination with atezolizumab | graded according to NCI CTCAE v4.0 |
| (Phase Ib), including estimation of the | Change from baseline in targeted vital signs |
| maximum tolerated dose (MTD) and the | Change from baseline in targeted clinical |
| recommended Phase II dose (RP2D) and | laboratory test results, including ECGs |
| characterization of dose-limiting toxicities | Number of cycles received and dose intensity |
| (DLTs) | Incidence of anti-tiragolumab antibodies (Phase Ia |
| Change from baseline in targeted vital signs | or Phase Ib) and/or anti-atezolizumab antibodies |
| To evaluate the safety and tolerability of | (Phase Ib) and the potential correlation with PK, |
| tiragolumab in combination with atezolizumab | pharmacodynamic, safety, and preliminary |
| and chemotherapy (Phase Ib chemotherapy | efficacy parameters |
| expansion cohorts). | |
| Pharmacokinetic Objective: | |
| To characterize the pharmacokinetic (PK) profile | Serum concentration of tiragolumab administered |
| of tiragolumab administered as a single agent | as a single agent (Phase Ia) or in combination |
| (Phase Ia) or in combination with atezolizumab | with atezolizumab (Phase Ib) with or without |
| with and without chemotherapy (Phase Ib) | chemotherapy at specified timepoints for the |
| To characterize the PK profile of atezolizumab | following parameters: |
| administered in combination with tiragolumab | Area under the concentration-time curve |
| with and without chemotherapy (Phase Ib only) | (AUC) |
| To characterize the PK profile of cisplatin, | Maximum serum concentration (Cmax) |
| carboplatin, pemetrexed, paclitaxel, and | Minimum serum concentration (Cmin) |
| etoposide when administered in combination | Clearance (CL) |
| with atezolizumab and tiragolumab (Phase Ib | Volume of distribution at steady-state |
| chemotherapy expansion cohorts) | (Vss) |
| | Other parameters such as accumulation ratio, |
| | half-life, and dose proportionality may also be |
| | calculated |
| | Serum Cmax and Cmin of atezolizumab |
| | Plasma concentrations of cisplatin, carboplatin, |
| | pemetrexed, paclitaxel, and etoposide at specified |
| | timepoints |
| Activity Objective: | |
| To make a preliminary assessment of the anti- | Objective response, defined as a complete |
| tumor activity of tiragolumab as a single agent | response (CR) or partial response (PR) per |
| (Phase Ia) or in combination with atezolizumab | Response Evaluation Criteria in Solid Tumors |
| with and without chemotherapy (Phase Ib) in | (RECIST) v.1.1, as determined by the investigator |
| patients with locally advanced or metastatic | and confirmed by repeat assessment ≥4 weeks |
| tumors | after initial documentation. |
| | Duration of objective response (DoR), defined as |
| | the time from the first occurrence of a |
| | documented objective response to the time of the |
| | first documented disease progression or death |
| | from any cause, whichever occurs first, per |
| | RECIST v1.1 as determined by the investigator |
| | PFS, defined as the time from the first study |
| | treatment to the first occurrence of progression or |
| | death from any cause, whichever occurs first, per |
| | RECIST v.1.1 as determined by the investigator |
| | Overall survival (OS), defined as the time from |
| | first study treatment to death from any cause. |
| Immunogenicity Objectives: | |
| To characterize the immunogenic potential of | Incidence of anti-drug antibodies (ADAs) during |
| tiragolumab administered as a single agent by | the study relative to the prevalence of ADAs at |
| measuring anti-tiragolumab antibodies and | baseline |
| assessing their relationship with other outcome | |
| measures (Phase Ia) or tiragolumab in | |
| combination with atezolizumab with and | |
| without chemotherapy by measuring anti- | |
| tiragolumab and/or anti-atezolizumab | |
| antibodies and assessing their relationship | |
| with other outcome measures (Phase Ib) | |

A. Study Design

This is a first-in-human Phase I open-label, multicenter, global, dose-escalation study designed to evaluate the safety, tolerability, and PK of tiragolumab as a single agent inpatients with locally advanced, recurrent, or metastatic incurable tumors for whom standard therapy does not exist, has proven to be ineffective or intolerable, or is considered inappropriate, or for whom a clinical trial of an investigational agent is a recognized standard of care. This study is also designed to enable evaluation of the safety, tolerability, and PK of tiragolumab when administered with atezolizumab with and without chemotherapy in patients with locally advanced, recurrent, or metastatic incurable tumors for whom standard therapy does not exist, has proven to be ineffective or intolerable, or is considered inappropriate, or for whom a clinical trial of an investigational agent is a recognized standard of care, or for whom a clinical trial of an investigational agent in combination with an anti-PD-L1 antibody is considered an acceptable treatment option.

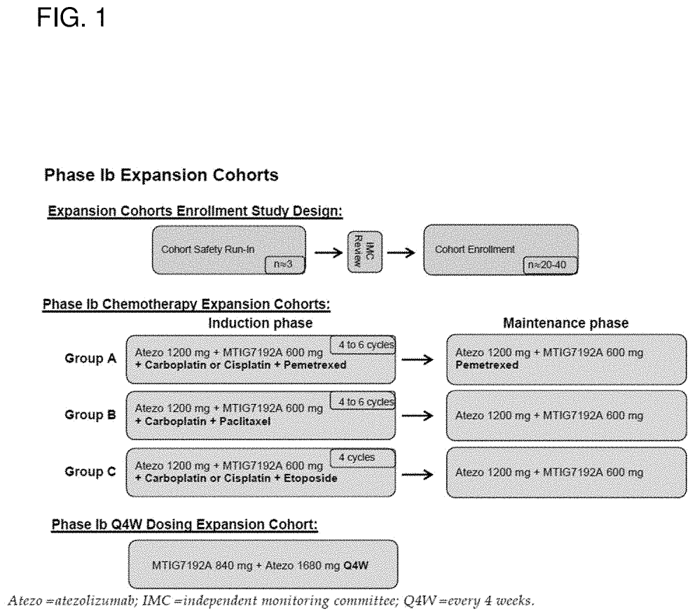

FIG. 1 is a flow chart showing the Phase Ib chemotherapy expansion and Phase Ib Q4W dosing expansion). During the dose-escalation stage, cohorts of approximately 3-6 patients each are evaluated at escalating dose levels to determine the MTD or maximum administered dose (MAD) for tiragolumab as a single agent or in combination with atezolizumab.

In the dose-expansion stage, patients are enrolled and treated at or below the MTD or MAD of tiragolumab as a single agent (Phase Ia), or in combination with atezolizumab with or without chemotherapy (Phase Ib). Tiragolumab as a single agent (Phase Ia) or the combination of tiragolumab and atezolizumab (Phase Ib cohorts without chemotherapy) is administered by IV infusion on Day 1 of each 21-day cycle or on Day 1 of each 28-day cycle (Phase Ib Q4W dosing expansion), with tiragolumab being administered prior to atezolizumab in the Phase Ib cohorts without chemotherapy. In the absence of unacceptable toxicity or clinically compelling evidence of disease progression, treatment with either tiragolumab (Phase Ia) or tiragolumab in combination with atezolizumab (Phase Ib) is continued beyond Cycle 1 based on a favorable assessment of benefit and risk by the investigator.

In the Phase Ib chemotherapy expansion cohorts and the Phase Ib Q4W dosing expansion cohort (FIG. 1), a safety run-in of 3 patients is completed. All relevant safety data from the safety run-in is thoroughly reviewed by an IMC and with the investigators before enrollment is continued.

In the chemotherapy expansion cohort, tiragolumab and atezolizumab is combined with specific chemotherapy regimens in each of the three cohorts: carboplatin or cisplatin and pemetrexed in Cohort A, carboplatin and paclitaxel in Cohort B and carboplatin or cisplatin and etoposide in Cohort C (see FIG. 1). In each cohort, treatment includes of an induction phase and a maintenance phase. In the induction phase, the combination of tiragolumab and atezolizumab with chemotherapy is administered by IV infusion on a 21-day cycle for 4 to 6 cycles for Cohorts A and B and for 4 cycles for Cohort C. The number of cycles of induction treatment for Cohort A and B is at the discretion of the investigator.

Following the induction phase, patients who have not experienced disease progression or unacceptable toxicity continue treatment with maintenance therapy. During the maintenance phase, patients in Cohorts B and C continue tiragolumab and atezolizumab only, while patients in Cohort A continue tiragolumab and atezolizumab with pemetrexed. In all the chemotherapy expansion cohorts, atezolizumab is administered prior to tiragolumab. When chemotherapy is given, it is administered after atezolizumab and tiragolumab.

All patients are closely monitored for adverse events throughout the study and for at least 90 days after the last dose of study treatment or until initiation of another systemic anti-cancer therapy, whichever occurs first. Adverse events are graded according to the NCI CTCAE, Version 4.0.

To characterize the PK properties, immunogenic response, and pharmacodynamic effects of tiragolumab as a single agent (Phase Ia) or in combination with atezolizumab with and without chemotherapy (Phase Ib), blood samples are taken at various timepoints before and after dosing. Depending on the results from the interim PK analyses, the frequency of PK sampling may be reduced later in the study.

Patients undergo tumor assessments at screening and during the study, which are measured by standard Response Evaluation Criteria in Solid Tumors (RECIST) v1.1 criteria. Patients may be permitted to continue study treatment even if standard RECIST v1.1 criteria for progression of disease are met in the Phase Ia or Phase Ib portions of the study, provided that they meet the criteria for continued treatment. Patients who discontinue the Phase Ia portion of the study may be permitted to cross over into the Phase Ib portion of the study and receive treatment with tiragolumab in combination with atezolizumab, provided that they meet the criteria for crossover and consent to a biopsy of an accessible lesion.

Approximately 60-320 patients with locally advanced, recurrent, or metastatic incurable malignancies that have progressed after available standard therapy; or for whom standard therapy has proven to be ineffective or intolerable, or is considered inappropriate; or for whom a clinical trial of an investigational agent is a recognized standard of care, are enrolled in the expansion cohorts of the study. For the Phase Ib portion of the study, patients for whom a clinical trial of an investigational agent in combination with an anti-PD-L1 antibody with or without chemotherapy is considered an acceptable treatment option may be enrolled in the expansion cohorts.

This expansion stage includes defined cohorts of patients to better characterize the safety, tolerability, PK variability, pharmacodynamic activity, and preliminary anti-tumor activity of tiragolumab as a single agent (Phase Ia) or in combination with atezolizumab with or without chemotherapy (Phase Ib) in specific cancer settings. Enrollment in the expansion cohorts is initiated at a selected dose level at or below the MAD or MTD of tiragolumab as a single agent (Phase Ia) or tiragolumab in combination with atezolizumab with or without chemotherapy (Phase Ib), as determined by the study investigators, based on an assessment of accumulating safety, tolerability, PK, pharmacodynamic, and anti-tumor activity data.

In the Phase Ia portion of the study, up to approximately 40 patients are enrolled in a planned expansion cohort of multiple tumor indications that are PD-L1-selected and/or TIGIT-selected, including NSCLC, RCC, TNBC, melanoma, HNSCC, OC, GC including GEJ cancer, UBC, and CRC, including CRC that is MSS or MSI-Low.

In the Phase Ib portion of the study (without chemotherapy), approximately 20-40 patients are enrolled in each of the following planned indication-specific expansion cohorts: NSCLC: Cancer immunotherapy (CIT)-Naive (e.g., no prior treatment with anti-PD-L1/PD-1); NSCLC: CIT-Treated (e.g., including prior treatment with anti-PD-L1/PD-1); RCC; TNBC; Melanoma; HNSCC; OC; GC, including GEJ cancer; UBC; CRC, including CRC that is MSS or MSI-Low; Biopsy cohort of specific tumor indications, including melanoma, OC, RCC, and UBC.

In the Phase Ib chemotherapy expansion portion of the study, approximately 20-40 patients are enrolled in each of the following planned chemotherapy expansion cohorts (See FIG. 1):

### Cohort A:

Induction phase—atezolizumab and tiragolumab in combination with cisplatin or carboplatin and pemetrexed
Maintenance phase—atezolizumab and tiragolumab in combination with Pemetrexed

### Cohort B:

Induction phase—atezolizumab and tiragolumab in combination with carboplatin and paclitaxel
Maintenance phase—atezolizumab and tiragolumab

### Cohort C:

Induction phase—atezolizumab and tiragolumab in combination with cisplatin or carboplatin and etoposide
Maintenance phase—atezolizumab and tiragolumab

The treatment combinations in each cohort are shown in Table 6.

TABLE 6
Treatment Regimens in the Chemotherapy Expansion Cohorts

| | Induction Phase | Maintenance Phase |
|---|---|---|
| Treatment Cohort | (Four or Six 21-day Cycles) | (21-day Cycles) |
| A | Atezolizumab + tiragolumab + | Atezolizumab + tiragolumab + |
| | Cisplatin or carboplatin + pemetrexed | pemetrexed |
| B | Atezolizumab + tiragolumab + | Atezolizumab + |
| | carboplatin + paclitaxel | tiragolumab |
| C | Atezolizumab + tiragolumab + | Atezolizumab + |
| | Cisplatin or carboplatin + etoposide | tiragolumab |

B. Phase Ib Chemotherapy Expansion Cohorts: Dosing and Administration

In the induction phase, patients in the specific chemotherapy expansion cohorts receive the following:

Cohort A receives atezolizumab 1200 mg IV, then tiragolumab 600 mg IV, followed by the combination of cisplatin 75 mg/m² IV or carboplatin AUC of 6 mg/mL·min IV and pemetrexed 500 mg/m² IV on Day 1 of an every 21-day cycle. Four to six cycles of induction-phase treatment are administered in the absence of disease progression or unacceptable toxicity.

Cohort B receives atezolizumab 1200 mg IV, then tiragolumab 600 mg IV, followed by the combination of carboplatin AUC of 6 mg/mL·min IV and paclitaxel 200 mg/m² IV on Day 1 of an every 21-day cycle. Four to six cycles of induction phase treatment are administered in the absence of disease progression or unacceptable toxicity.

Cohort C receives atezolizumab 1200 mg IV, then tiragolumab 600 mg IV followed by cisplatin 75 mg/m2 IV or carboplatin AUC of 5 mg/mL·min IV on Day 1 of an every 21-day cycle and then etoposide 100 mg/m² IV on Days 1-3 of an every 21-day cycle. Four cycles of induction phase treatment are administered in the absence of disease progression or unacceptable toxicity.

Following the induction phase, treatment continues in the maintenance phase in the absence of unacceptable toxicity, clinically compelling disease progression, and/or loss of clinical benefit at the investigator's discretion following a careful assessment and thorough discussion of the potential risks and benefits with the patient. In the maintenance phase, patients in the specific chemotherapy expansion cohorts receive the following:

Cohort A receives atezolizumab 1200 mg IV, then tiragolumab 600 mg IV, followed by pemetrexed 500 mg/m² IV on Day 1 of an every-21-day cycle; Cohort B receives atezolizumab 1200 mg IV and then tiragolumab 600 mg IV on Day 1 of an every-21-day cycle; Cohort C receives atezolizumab 1200 mg IV and then tiragolumab 600 mg IV on Day 1 of an every-21-day cycle.

In the event of toxicity and the absence of disease progression, individual chemotherapy or immunotherapy agents are independently discontinued.

C. Phase Ib Q4W Dosing Expansion Cohort

The objectives of the Phase Ib Q4W dosing expansion cohort are to better characterize the safety, tolerability, PK, and preliminary efficacy data and to explore potential tumor biomarkers of pharmacodynamic activity in patients treated with tiragolumab 840 mg IV in combination with atezolizumab 1680 mg IV with an every 4 week (28 day) dosing schedule.

The Phase Ib Q4W cohort includes approximately 20-40 patients with tumors that can be PD-L1-selected and/or TIGIT-selected based on prospective testing of tumor tissue during screening or rescreening. A patient with insufficient or unavailable archival tissue may be eligible for enrollment in this cohort, if deemed so by the Medical Monitor based upon a discussion with the investigator. Patients with a tumor type for whom a clinical trial of an investigational agent in combination with an anti-PD-L1 antibody is considered an acceptable treatment option may be enrolled in these expansion cohorts.

D. Additional Inclusion Criteria for Patients in Each Indication-Specific Expansion Cohort of Phase Ib

The NSCLC Cohort (CIT-naive) includes patients with histologically confirmed incurable, advanced NSCLC not previously treated with CIT (investigational or approved), including anti-PD-L1/PD-1 and/or anti-CTLA-4, for whom a clinical trial of an investigational agent in combination with an anti-PD-L1 antibody is considered an acceptable treatment option, if CIT (including anti-PD-L1/PD-1 agents) is approved as treatment for NSCLC by local regulatory authorities. Patients whose tumors have a known sensitizing epidermal growth factor receptor (EGFR) mutation must also have experienced disease progression (during or after treatment) or intolerance to treatment with an EGFR tyrosine kinase inhibitor(s). Patients whose tumors have a known anaplastic lymphoma kinase (ALK) rearrangement must also have experienced disease progression (during or after treatment) or intolerance to treatment with an ALK tyrosine kinase inhibitor(s). Patients whose tumors have a known ROS1 rearrangement must also have experienced disease progression (during or after treatment) or intolerance to treatment with an ROS1 tyrosine kinase inhibitor(s). Patients whose tumors have a BRAFV600E mutation must also have experienced disease progression (during or after treatment) or intolerance to treatment with dabrafenib in combination with trametinib.

The NSCLC cohort (CIT-treated) includes patients with histologically confirmed incurable, advanced NSCLC previously treated with CIT (investigational or approved) including anti-PD-L1/PD-1. Patients whose tumors have a known sensitizing EGFR mutation must also have experienced disease progression (during or after treatment) or intolerance to treatment with EGFR tyrosine kinase inhibitor(s). Patients whose tumors have a known ALK rearrangement must also have experienced disease progression (during or after treatment) or intolerance to treatment with an ALK tyrosine kinase inhibitor(s). Patients whose tumors have a known ROS1 rearrangement must also have experienced disease progression (during or after treatment) or intolerance to treatment with an ROS1 tyrosine kinase inhibitor(s). Patients whose tumors have a BRAFV600E mutation must also have experienced disease progression (during or after treatment) or intolerance to treatment with dabrafenib in combination with trametinib. Patients must have experienced documented disease progression on CIT monotherapy and/or combination therapy (investigational or approved), which must have included a prior anti-PD-L1/PD-1.

At least approximately 10 patients who experienced a documented best response of investigator-assessed confirmed PR or CR per RECIST v1.1 at any time while receiving the prior anti-PD-L1/PD-1 as monotherapy or combination therapy may be enrolled. At least approximately 10 patients who experienced a documented best response of investigator-assessed SD per RECIST v1.1 at any time while receiving the prior anti-PD-L1/PD-1 as monotherapy and/or as combination therapy may be enrolled. At least approximately 10 patients who experienced a documented best response of investigator-assessed progressive disease (PD) per RECIST v1.1 at any time while receiving the prior anti-PD-L1/PD-1 as monotherapy and/or as combination therapy may be enrolled. The prior anti-PD-L1/PD-1 as monotherapy and/or as combination therapy must represent the most recent systemic anti-cancer therapy administered prior to enrollment in this expansion cohort. Patients who discontinued the prior anti-PD-L1/PD-1 monotherapy and/or combination therapy primarily for toxicity or intolerability are not eligible for enrollment in this expansion cohort.

The TNBC cohort includes patients with histologically confirmed incurable, advanced estrogen receptor (ER)-negative, progesterone receptor (PgR)-negative, and human EGFR 2 (HER2)-negative adenocarcinoma of the breast (triple-negative). Triple-negative status must be documented as defined by the American Society of Clinical Oncology College of American Pathologists (ASCO-CAP) guidelines: <1% of tumor cell nuclei are immunoreactive for ER and <1% of tumor cell nuclei are immunoreactive for progesterone receptor and HER2 tests demonstrate IHC 1+, IHC 0, or in situ hybridization (ISH) negative

The CRC cohort includes patients with histologically confirmed incurable, advanced adenocarcinoma of the colon or rectum. Patients with tumors of appendiceal origin are not eligible.

The GC cohort includes patients with histologically confirmed inoperable, locally advanced or metastatic or recurrent gastric or GEJ adenocarcinoma, not amenable to curative therapy. Patients with Type 1 GEJ tumor, defined by Rudiger Siewert et al. (2000) as adenocarcinoma of the distal esophagus with the tumor center located within 1 to 5 cm above the anatomic esophagogastric junction, are eligible for the study. Patients with esophageal cancers (squamous cell carcinoma or adenocarcinoma) may be eligible following a discussion with the Medical Monitor. Patients whose tumors are HER2-positive must also have experienced disease progression (during or after treatment) or intolerance to treatment with HER2-targeting antibody/HER2 inhibitor(s). HER2-positivity is defined as either IHC 3+ or IHC 2+/ISH+ (where ISH positivity is defined as a HER2:CEP17 ratio of ≥2), as assessed by a local laboratory test on the primary tumor or on a metastatic lesion. Patients who have not had HER2 testing due to insufficient or unavailable tissue (e.g., archival and/or biopsy), and thus the HER2 status of the tumor is unknown, may still be eligible.

The HNSCC cohort includes patients with histologically confirmed inoperable, locally advanced or metastatic, recurrent, or persistent head and neck squamous cell carcinoma (oral cavity, oropharynx, hypopharnyx, or larynx), not amenable to curative therapy. Patients with HNSCC of any other primary anatomic location in the head and neck, patients with HNSCC of unknown primary, or patients with tumors of non-squamous histologies are not eligible. Patients with HNSCC of the nasopharynx may be eligible. HPV status for the HNSCC must be known.

The UBC cohort includes patients with histologically confirmed incurable advanced transitional cell carcinoma of the urothelium (including renal pelvis, ureters, urinary bladder, and urethra). Patients with mixed histologies are required to have a dominant transitional cell pattern.

The melanoma cohort includes patients with histologically confirmed incurable, advanced metastatic melanoma. Patients with melanoma for whom a clinical trial of an investigational agent in combination with an anti-PD-L1 antibody is considered an acceptable treatment option, if CIT (including anti-PD-L1/PD-1 agents and/or anti-CTLA-4 agents) is approved as treatment for melanoma by local regulatory authorities. Patients whose tumors have a known BRAFV600 mutation must also have experienced disease progression (during or after treatment) or intolerance with BRAF inhibitor(s) and/or MEK inhibitor(s). Enrollment is managed so that no more than approximately 20% of patients in this cohort are patients with ocular (uveal) melanoma.

The OC cohort includes patients with histologically confirmed incurable, advanced epithelial ovarian, fallopian tube, or primary peritoneal cancer. Borderline ovarian epithelial neoplasms (e.g., tumors of low malignant potential, atypical proliferative tumors) are excluded.

The RCC cohort includes patients with histologically confirmed incurable, advanced RCC with component of clear cell histology and/or component of sarcomatoid histology. Patients with RCC for whom a clinical trial of an investigational agent in combination with an anti-PD-L1 antibody is considered an acceptable treatment option, if CIT (including anti-PD-L1/PD-1 agents) is approved as treatment for RCC by local regulatory authorities.

E. Dosing and Administration

The dose of atezolizumab administered in combination with tiragolumab in the Phase Ib portion of this study is 1200 mg IV every three weeks, except in the Phase Ib Q4W dosing cohort where atezolizumab 1680 mg IV Q4W is administered. This dose is fixed and not dependent on body weight. In all Phase Ib cohorts without chemotherapy, atezolizumab is administered after the tiragolumab infusion and subsequent observation period. In the Phase Ib chemotherapy expansion cohorts, atezolizumab is administered before tiragolumab.

The initial dose of atezolizumab is delivered over 60 (±10) minutes. If the first infusion is tolerated without infusion-associated adverse events, the second infusion may be delivered over 30 (±10) minutes. If the 30-minute infusion is well tolerated, all subsequent infusions may be delivered over 30 (±10) minutes. For Cycle 1, dosing of atezolizumab is followed by a 90-minute observation period. All subsequent infusions of atezolizumab may be followed by a 30-minute observation period. Patients who have previously received atezolizumab on another clinical trial may receive the initial dose at the fastest rate that was previously tolerated.

### Chemotherapy in the Phase Ib Expansion Cohorts

Chemotherapy is administered after the atezolizumab and tiragolumab infusions and subsequent observation periods. During the induction phase, a chemotherapy cycle counts toward the prespecified number of induction chemotherapy cycles as long as at least one chemotherapy component has been administered at least once during a 21-day cycle. Cycles in which no chemotherapy component is given do not count toward the total number of induction chemotherapy cycles.

Patients receive anti-emetics and IV hydration for chemotherapy agents according to the local standard-of-care and manufacturer's instruction. However, because of the immunomodulatory effects of steroids, premedication with steroids should be minimized to the extent that is clinically feasible.

### Cohort A—Atezolizumab Plus Tiragolumab Plus Carboplatin/Cisplatin Plus Pemetrexed

On Day 1 of each 21 day cycle, all eligible patients receive drug infusions in the following order:

Induction: Atezolizumab>tiragolumab>pemetrexed>carboplatin or cisplatin

After 4 to 6 cycles in the induction phase, patients begin maintenance therapy in the following order of administration:

Maintenance: Atezolizumab >tiragolumab >pemetrexed

TABLE 7
Treatment Regimen for Pemetrexed and Carboplatin or Cisplatin

| | Dose and | Induction Period (Four | |
|---|---|---|---|
| Study Drug | Route | to Six Cycles) | Maintenance Period |
| Pemetrexed | 500 mg/ | ~10-minute infusion on | ~10-minute infusion on |
| | m² IV | Day 1 Q3W | Day 1 Q3W |
| Carboplatin | AUC 6 IV | ~30-60 minute infusion | Not applicable |
| | | on Day 1 Q3W | |

or

| | | | |
|---|---|---|---|
| Cisplatin | 75 mg/ | 1-2 hour infusion on | Not applicable |
| | m² IV | Day 1 Q3W | |

AUC = area under the concentration-time curve; IV = intravenous; Q3W = every 3 weeks.

Table 8 lists suggested infusion times for treatment administration for pemetrexed and carboplatin or cisplatin during the induction phase and for pemetrexed during the maintenance phase.

TABLE 8
Premedication for Paclitaxel

| Premedication | Dose and Route | Timing |
|---|---|---|
| Dexamethasone | 20 mg PO | 12 and 6 hours before paclitaxel |
| | | (or per standard of care at |
| | | treating institution) |
| Diphenhydramine | 50 mg IV | 30-60 minutes before paclitaxel |
| (or equivalent) | | |
| Cimetidine | 300 mg (or 50 mg) | 30-60 minutes before paclitaxel |
| (or rantidine) | IV (or equivalent) | |
| (or equivalent) | | |

IV = intravenous; PO = orally

### Cohort B—Atezolizumab Plus Tiragolumab Plus Carboplatin Plus Paclitaxel

On Day 1 of each 21-day cycle, all eligible patients receive drug infusions in the following order:

Induction: Atezolizumab >tiragolumab >paclitaxel >carboplatin

Table 8 lists the suggested premedication for induction treatment for patients in Cohort B. Table 9 lists the suggested infusion times for treatment administration for paclitaxel and carboplatin during the induction phase.

TABLE 9
Treatment Regimen for Paclitaxel and Carboplatin

| | | Induction Period |
|---|---|---|
| Study Drug | Dose and Route | (4 to 6 Cycles) |
| Paclitaxel | 200 mg/m² IV (175 mg/ | Over approximately 180 |
| | m2 IV for patients of | minutes on Day 1 Q3W |
| | Asian race/ethnicity)^{a} | |
| Carboplatin | AUC 6 IV | Over approximately 30-60 |
| | | minutes on Day 1 Q3W |

AUC = area under the concentration-time curve; IV = intravenous; Q3W = every 3 weeks.

### Cohort C—Atezolizumab Plus Tiragolumab Plus Carboplatin or Cisplatin Plus Etoposide

On Day 1 of each 21-day cycle, all eligible patients are administered study drug infusions in the following order:

Induction: Atezolizumab >tiragolumab >cisplatin or carboplatin >etoposide

After the induction phase, patients begin maintenance therapy in the following order of administration:

Maintenance: Atezolizumab >tiragolumab

Table 10 lists the suggested infusion times for treatment administration for carboplatin or cisplatin and etoposide during the induction phase.

TABLE 10
Treatment Regimen for Carboplatin or Cisplatin and Etoposide

| Study Drug | Dose and Route | Induction Period (4 Cycles) |
|---|---|---|
| Carboplatin | AUC 5 IV | Over 30-60 minutes on Day 1 Q3W |

Or

| | | |
|---|---|---|
| Cisplatin | 75 mg/m² IV | Over 60-120 minutes on Day 1 Q3W |
| Etoposide | 100 mg/m² IV | Over 60 minutes on Day 1, Day 2, |
| | | and Day 3 Q3W |

IV = intravenous; Q3W = every 3 weeks.

### Cisplatin

Guidelines for the administration of cisplatin in different cohorts are shown in Table 11.

TABLE 11
Cisplatin Administration Timing and Guidelines

| Chemotherapy | | | |
|---|---|---|---|
| Expansion Cohort | Timing | Dose and Route | Duration |
| A | Day 1: 30 minutes after completion of | 75 mg/m² IV | 60-120 minutes |
| | pemetrexed | | |
| C | Day 1: After completion of tiragolumab | 75 mg/m² IV | 60-120 minutes |
| | observation period | | |

IV = intravenous

Note: Patients must receive adequate anti-emetic treatment and appropriate hydration prior to and after receiving cisplatin. Refer to local clinical practice guidelines for further details.

### Carboplatin

Guidelines for the administration of cisplatin in different cohorts are shown in Table 12.

TABLE 12
Carboplatin Administration Timing and Guidelines

| Chemotherapy | | | |
|---|---|---|---|
| Expansion Cohort | Timing | Dose and Route | Duration |
| A | Day 1: 30 minutes | 6 mg/mL/min IV | 30-60 minutes |
| | after completion of | | |
| | pemetrexed | | |
| B | Day 1: after | AUC 6 mg/mL/ | 30-60 minutes |
| | completion of | min IV | |
| | paclitaxel | | |
| C | Day 1: after | AUC 5 mg/mL/ | 30-60 minutes |
| | completion of | min IV | |
| | tiragolumab | | |
| | observation period | | |

AUC= area under time-concentration curve; IV = intravenous
Note:
Standard antiemetics should be given with carboplatin administration per local practice guidelines, the carboplatin dose of AUC 5 or 6 is calculated using Calvert formula (Calvert et al., J. Clin. Oncol. 1989, 7:1748-56):

For the purposes of this study, the GFR is considered to be equivalent to the calculated creatinine clearance (CrCl). The CrCl is calculated by institutional guidelines or by the method of Cockcroft and Gault (1976) using the following formula:

$$CrCl = \frac{( 140 - age ) ( wt )}{72 \times Scr} ( \times 0.85 if female )$$

Where: CrCl=creatinine clearance in mL/min
   age=patient's age in years
   wt=patient's weight in kg
   Scr=serum creatinine in mg/dL
      For patients with an abnormally low serum creatinine level, estimate the GFR through use of a minimum creatinine level of 0.8 mg/dL or cap the estimated GFR at 125 mL/min.

If a patient's GFR is estimated based on serum creatinine measurements by the isotope dilution mass spectroscopy method, the U.S. FDA recommends that physicians consider capping the dose of carboplatin for desired exposure (AUC) to avoid potential toxicity due to overdosing. Based on the Calvert formula described in the carboplatin label, the maximum doses can be calculated as follows:

Maximum carboplatin dose (mg)=target AUC (mg·min/mL)×(GFR+25 mL/min).

The maximum dose is based on a GFR estimate that is capped at 150 mL/min for patients with normal renal function. No higher estimated GFR values should be used.
For a target AUC=6, the maximum dose is 6×150=900 mg.
For a target AUC=5, the maximum dose is 5×150=750 mg.
For a target AUC=4, the maximum dose is 4×150=600 mg.

### Pemetrexed

Guidelines for the administration of pemetrexed in Cohort A is shown in Table 13.

TABLE 13
Pemetrexed Administration Timing and Guidelines

| Chemotherapy | | | |
|---|---|---|---|
| Expansion Cohort | Timing | Dose and Route | Duration |
| A | Day 1: after completion | 500 mg/m² IV | 10 minutes |
| | of tiragolumab | | |
| | observation period | | |

IV = intravenous

Premedication doses administered complies with the prescribing information. All patients eligible for pemetrexed therapy should avoid taking non-steroidal anti-inflammatory drugs with long elimination half-lives for at least 5 days prior to, on the day of, and at least 2 days following pemetrexed administration.

### Paclitaxel

Guidelines for the administration of paclitaxel in Cohort B is shown in Table 14.

TABLE 14
Paclitaxel Administration Timing and Guidelines

| Chemotherapy | | | |
|---|---|---|---|
| Expansion Cohort | Timing | Dose and Route | Duration |
| B | Day 1: after completion of | 200 mg/m² IV | 3 hours |
| | tiragolumab observation period | (175 mg/m² IV for patients of | |
| | | Asian race/ethnicity) | |

IV = intravenous

Patients of Asian race/ethnicity have a lower starting dose of paclitaxel at 175 mg/m2 IV over 3 hours. The lower starting dose of paclitaxel is based on a higher overall incidence of hematologic toxicities in patients from Asian countries compared with those from non-Asian countries, as observed during internal safety review of lung cancer clinical trials. As used in this study, Asian race/ethnicity refers to a panethnic/racial group that includes diverse populations who either live or have ancestral origins in East Asia, Southeast Asia, or South Asia. The applicability of such term in a particular patient is at the discretion of the treating investigator and should be based on the patient's clinical characteristics and country of origin.

### Etoposide

Guidelines for the administration of etoposide in Cohort C is shown in Table 15.

TABLE 15
Etoposide Administration Timing and Guidelines

| Chemotherapy | | | |
|---|---|---|---|
| Expansion Cohort | Timing | Dose and Route | Duration |
| C | Day 1: after completion of | 100 mg/m² IV | 60 minutes |
| | cisplatin/carboplatin | | |
| | Days 2 and 3: Only | 100 mg/m² IV | 60 minutes |
| | etoposide is delivered on | | |
| | Days 2 and 3 | | |

IV = intravenous

### Example 2. A Phase Ib/II, Open-Label, Randomized Study of Atezolizumab and Chemotherapy in Combination with Tiragolumab

This study evaluates the efficacy, safety, and pharmacokinetics of atezolizumab and chemotherapy (nanoparticle albumin-bound paclitaxel (nab-paclitaxel) and gemcitabine) in combination with tiragolumab in patients who have received no prior systemic therapy for metastatic pancreatic ductal adenocarcinoma (PDAC). This study evaluates the efficacy, safety, and pharmacokinetics of immunotherapy-based treatment combinations in patients with metastatic PDAC.

A. Study Design

Patients in Cohort 1 are randomly assigned to a control arm (chemotherapy) or an experimental arm consisting of atezolizumab and chemotherapy in combination with tiragolumab. Enrollment within the experimental arms takes place in two phases: a preliminary phase followed by an expansion phase. Approximately 20 patients are enrolled during the preliminary phase. Randomization is suspended to allow for a safety evaluation in a minimum of 6 patients. The safety evaluation is based on safety data from a minimum of 6 patients who have received at least one dose of treatment (i.e., one dose of each agent for a given combination) and completed safety follow-up assessments during at least one full treatment cycle. If the combination is determined to be sufficiently safe, enrollment is resumed in that arm. If clinical activity is observed in an experimental arm during the preliminary phase, approximately 25 additional patients may be enrolled in that arm during the expansion phase. Additional patients may be enrolled to ensure balance among treatment arms with respect to demographic and baseline characteristics, including potential predictive biomarkers, to enable further subgroup analyses.

Patients are randomly assigned to treatment arms, and the randomization ratio depends on the number of experimental arms that are open for enrollment (e.g., if an arm is added or enrollment in an arm is suspended pending analysis of results from the preliminary phase), with the stipulation that the likelihood of being allocated to the control arm is no more than 35%.

The end of this study is defined as the date when the last patient completes the last visit (LPLV), including survival follow-up visits conducted by telephone or in the clinic. The total length of the study, from screening of the first patient to the end of the study, will be approximately 3-5 years.

A schedule of the activities is outlined in Table 16.

TABLE 16
Schedule of Activities

| | Treatment Cycles (28-day cycles)^{a} | | | | | | | |
|---|---|---|---|---|---|---|---|---|
| | Cycle 1^{b} | | | Cycle ≥ 2 | | | | Follow-Up^{c} |
| | | Day 8 | Day 15 | | Day 8 | Day 15 | Treat. | Every 3 |
| | Day 1 | (±3 days) | (±3 days) | Day 1 | (±3 days) | (±3 days) | Discon.^{c} | Months |
| Molecular profile | Whenever updated information becomes available | | | | | | | |
| of pancreatic | | | | | | | | |
| cancer | | | | | | | | |
| (if available) | | | | | | | | |
| Vital signs | X | X | X | X | X | X | X | |
| Weight | X | X | X | X | X | X | X | |
| Complete physical | | | | | | | X | |
| examination^{e} | | | | | | | | |
| Limited physical | X | X | X | X | X | X | | |
| examination^{f} | | | | | | | | |
| ECOG | X | | | X | | | X | |
| Performance | | | | | | | | |
| Status | | | | | | | | |
| ECG | Perform as clinically indicated | | | | | | | |
| Hematology^{h} | X^{i,j} | Xⁱ | Xⁱ | Xⁱ | Xⁱ | Xⁱ | X | |
| Chemistry^{k} | X^{i,j} | Xⁱ | Xⁱ | Xⁱ | Xⁱ | Xⁱ | X | |
| Coagulation | X^{i,j} | Perform as clinically indicated | | | | | X | |
| (INR and aPTT) | | | | | | | | |
| TSH, free T3 | X^{i,j,l} | | | | | | X | |
| (or total T3), free T4^{l} | | | | | | | | |
| Viral serology | | | | X^{m} | | | X^{m} | X^{m} |
| (if applicable) | | | | | | | | |
| C-reactive protein | X^{i,j} | | | Xⁱ | | | | |
| CA19-9 | Xⁱ | | | Xⁱ | | | X | |
| Pregnancy test | X^{i,j} | | | Xⁱ | | | X | Xⁿ |
| Urinalysis^{o} | Perform as clinically indicated | | | | | | | |
| Serum | Perform as clinically indicated^{q} | | | | | | | |
| autoantibody | | | | | | | | |
| sample^{p} | | | | | | | | |
| PK samples | Refer to Table 17 | | | | | | | |
| ADA samples | Refer to Table 17 | | | | | | | |
| Biomarker | Refer to Table 17 | | | | | | | |
| samples | | | | | | | | |
| Blood sample for | X | | | | | | | |
| RBR (optional)^{r} | | | | | | | | |
| Tumor biopsy | X^{s} | | | | | | | |
| Tumor biopsy | X^{t} | | | | | | | |
| (optional) | | | | | | | | |
| Tumor response | | | | | X^{u,v} | | | |
| assessments | | | | | | | | |
| Concomitant | X | X | X | X | X | X | X | |
| medications^{w} | | | | | | | | |
| Adverse events^{x} | X | X | X | X | X | X | X^{x} | X^{x} |
| Atezolizumab | X | | X | X | | X | | |
| administration^{y,z} | | | | | | | | |
| Tiragolumab | X | | X | X | | X | | |
| administration^{z,aa} | | | | | | | | |
| Nab-paclitaxel | X | X | X | X | X | X | | |
| and gemcitabine | | | | | | | | |
| administration^{z,bb} | | | | | | | | |
| Survival follow-up | | | | | | | | X^{cc} |
| and anti-cancer | | | | | | | | |
| treatment | | | | | | | | |

ADA = anti-drug antibody; Atezo + Chemo + Tira = atezolizumab plus chemotherapy (nab-paclitaxel and gemcitabine) plus tiragolumab; CIT = cancer immunotherapy; CT = computed tomography; Discon. = discontinuation; ECOG = Eastern Cooperative Oncology Group; eCRF = electronic Case Report Form; HBV = hepatitis B virus; nab-paclitaxel = nanoparticle albumin = bound paclitaxel; PK = pharmacokinetic; RBR = Research Biosample Repository; RECIST v1.1 = Response Evaluation Criteria in Solid Tumors, Version 1.1; T3 = triiodothyronine; T4 = thyroxine; Treat. = treatment; TSH = thyroid-stimulating hormone. On treatment days, all assessments should be performed prior to dosing, unless otherwise specified.
^{a}If a visit is precluded because of a holiday, vacation, or other circumstance, it can occur outside of the specified window if Medical Monitor agreement has been obtained.
^{b}It is recommended that treatment be initiated no later than 7 days after randomization.
^{c}Patients return to the clinic for a treatment discontinuation visit not more than 30 days after the last dose of study treatment. The visit at which disease progression is confirmed may be used as the treatment discontinuation visit. Patients then undergo follow-up assessments.
^{d}Vital signs include respiratory rate, pulse rate, and systolic and diastolic blood pressure while the patient is in a seated position, pulse oximetry, and temperature. Record new or worsened clinically significant abnormalities on the Adverse Event eCRF. For the first infusion of atezolizumab, vital signs should be measured within 60 minutes prior to the infusion and, if clinically indicated, every 15 (±5) minutes during and 30 (±10) minutes after the infusion. For subsequent infusions, vital signs should be measured within 60 minutes prior to the infusion and, if clinically indicated or if symptoms occurred during the previous infusion, during and 30 (±10) minutes after the infusion. For the first infusion of tiragolumab, vital signs should be measured within 60 minutes prior to the infusion and every 15 (±5) minutes during and 30 (±10) minutes after the infusion. For subsequent infusions of tiragolumab, vital signs should be measured within 60 minutes prior to the infusion and, if clinically indicated or if symptoms occurred during the previous infusion, during and 15 (±10) minutes after the infusion.
^{e}Complete physical examination includes evaluation of the head, eyes, ears, nose, and throat, and the cardiovascular, dermatologic, musculoskeletal, respiratory, gastrointestinal, genitourinary, and neurologic systems. Record new or worsened clinically significant abnormalities on the Adverse Event eCRF.
^{f}Perform a limited, symptom-directed examination at specified timepoints and as clinically indicated at other timepoints. Record new or worsened clinically significant abnormalities on the Adverse Event eCRF.
^{g}It is recommended that patients be resting in a supine position for at least 10 minutes prior to ECG recording.
^{h}Hematology includes WBC count, RBC count, hemoglobin, hematocrit, platelet count, and differential count (neutrophils, eosinophils, basophils, monocytes, lymphocytes, other cells).
ⁱLaboratory tests must be performed within 96 hours prior to Day 1 of Cycle 1 and within 24 hours prior to specified subsequent visits during the treatment period.
^{j}If screening laboratory assessments were performed within 96 hours prior to Day 1 of Cycle 1, they do not have to be repeated.
^{k}Chemistry panel (serum or plasma) includes bicarbonate or total carbon dioxide (if considered standard of care for the region), sodium, potassium, magnesium, chloride, glucose, BUN or urea, creatinine, total protein, albumin, phosphorus, calcium, total bilirubin, ALP, ALT, and AST.
^{l}TSH, free T3 (or total T3 for sites where free T3 is not performed), and free T4 are assessed at screening and on Day 1 of Cycle 1 and every third cycle thereafter (i.e., Cycles 4,7, 10, etc.).
^{m}Patients with a positive quantitative HBV DNA at screening (must be <500 IU/mL per the eligibility criteria) undergo additional HBV DNA tests on Day 1 of every third cycle (i.e., Cycles 3, 6, 9, etc.), at treatment discontinuation (±7 days), and at 3, 6, 9, and 12 months (±14 days at each timepoint) after treatment discontinuation. Study treatment and procedures may proceed while HBV DNA is being processed, but results should be reviewed by the investigator as soon as they are available. If HBV DNA increases to <500 IU/mL, consultation with the Medical Monitor is required prior to continuation of study treatment and consultation with a hepatologist or gastroenterologist with specialty in hepatitis B is recommended.
ⁿAll women of childbearing potential have urine or serum pregnancy tests performed at specified visits during treatment and at 3 months and 6 months after the last dose of study treatment. If a urine pregnancy test is positive, it must be confirmed by a serum pregnancy test.
^{o}Includes pH, specific gravity, glucose, protein, ketones, and blood; dipstick permitted.
^{p}Autoantibody analysis includes anti-nuclear antibody, anti-double-stranded DNA, circulating anti-neutrophil cytoplasmic antibody, and perinuclear anti-neutrophil cytoplasmic antibody.
^{q}Autoantibody analysis should be repeated for patients who develop signs or symptoms suggestive of autoimmune disease (e.g., lupus erythematosus).
^{r}Not applicable for a site that has not been granted approval for RBR sampling. Performed only for patients at participating sites who have provided written informed consent to participate.
^{s}Patients undergo tumor biopsy sample collection at the time of unacceptable toxicity or loss of clinical benefit as determined by the investigator, if deemed clinically feasible by the investigator. Biopsies should be performed within 40 days after determination of unacceptable toxicity or loss of clinical benefit, or prior to the next anti-cancer therapy, whichever is sooner. In addition, patients enrolled during the expansion phase undergo tumor biopsy sample collection 4 weeks (±7 days) after treatment initiation (if deemed clinically feasible), unless on-treatment tissue samples have already been collected, and determined to be evaluable, from a minimum of 15 patients treated with the same CIT combination.
^{t}Patients who consent to optional biopsies undergo tumor biopsy sample collection 4 weeks (±7 days) after treatment initiation, if deemed clinically feasible (does not apply to patients enrolled during the expansion phase who are already undergoing an on-treatment biopsy) and may undergo additional on-treatment biopsies at any other time at the investigator's discretion.
^{u}Patients undergo tumor assessments at baseline, every 6 weeks (±1 week) for the first 48 weeks following treatment initiation, and every 12 weeks (±2 weeks) thereafter, regardless of dose delays, until radiographic disease progression according to RECIST v1.1, except in the case of patients who continue treatment after radiographic disease progression; such patients undergo tumor assessments every 6 weeks (±1 week) until loss of clinical benefit as determined by the investigator. Thus, tumor assessments are to continue according to schedule in patients who discontinue treatment for reasons other than disease progression, even if they start new non-protocol-specified anti-cancer therapy.
^{v}All measurable and/or evaluable lesions identified at baseline should be re-assessed at each subsequent tumor evaluations according to the tumor assessment schedule described above (see footnote “t”). The same radiographic procedures used to assess disease sites at screening should be used for subsequent tumor assessments (e.g., the same contrast protocol for CT scans).
^{w}Includes any medication (e.g., prescription drugs, over-the-counter drugs, vaccines, herbal or homeopathic remedies, nutritional supplements) used by a patient in addition to protocol-mandated study treatment from 10 days prior to initiation of study treatment until the treatment discontinuation visit.
^{x}After initiation of study treatment, all adverse events are reported until 30 days after the last dose of study treatment or until initiation of new systemic anti-cancer therapy, whichever occurs first, and serious adverse events and adverse events of special interest continue to be reported until 135 days after the last dose of study treatment or until initiation of new systemic anti-cancer therapy, whichever occurs first. After this period, all deaths, regardless of cause, should be reported. The investigator should follow each adverse event until the event has resolved to baseline grade or better, the event is assessed as stable by the investigator, the patient is lost to follow-up, or the patient withdraws consent. Every effort should be made to follow all serious adverse events considered to be related to study treatment or trial-related procedures until a final outcome can be reported.
^{y} Atezolizumab is administered by IV infusion at a fixed dose of 840 mg on Days 1 and 15 of each 28-day cycle. The initial dose of atezolizumab is delivered over 60 (±15) minutes. Subsequent infusions are delivered over 30 (±10) minutes if the previous infusion was tolerated without infusion-associated adverse events, or 60 (±15) minutes if the patient experienced an infusion-associated adverse event with the previous infusion.
^{z}Treatment continues until unacceptable toxicity or loss of clinical benefit as determined by the investigator.
aaTiragolumab is administered by IV infusion at a fixed dose of 420 mg on Days 1 and 15 of each 28-day cycle. The initial dose of tiragolumab is delivered over 60 (±10) minutes. Subsequent infusions are delivered over 30 (±10) minutes if the previous infusion was tolerated without infusion-associated adverse events, or 60 (±10) minutes if the patient experienced an infusion-associated adverse event with the previous infusion. On Day 1 of Cycle 1, tiragolumab is administered 60 minutes after completion of the atezolizumab infusion. The interval between subsequent infusions is 30 minutes if the previous atezolizumab infusion was tolerated without an IRR or 60 minutes if the patient experienced an IRR with the previous atezolizumab infusion.
^{bb}On Days 1, 8, and 15, patients receive nab-paclitaxel 125 mg/m2, administered by IV infusion over 30 (±5) minutes, followed by gemcitabine 1000 mg/m2, administered by IV infusion over 30 (±5) minutes. On Day 1 of Cycle 1, nab-paclitaxel is administered 60 minutes after completion of the tiragolumab infusion to allow for observation after tiragolumab administration. The interval between subsequent infusions is 30 minutes if the previous tiragolumab infusion was tolerated without an IRR or 60 minutes if the patient experienced an IRR with the previous tiragolumab infusion.
^{cc}After treatment discontinuation, information on survival follow-up and new anti-cancer therapy (including targeted therapy and immunotherapy) is collected via telephone calls, patient medical records, and/or clinic visits approximately every 3 months until death (unless the patient withdraws consent or the study is terminated). If a patient requests to be withdrawn from follow-up, this request must be documented in the source documents and signed by the investigator. If the patient withdraws from the study, the study staff may use a public information source (e.g., county records) to obtain information about survival status only.

### Assessments and Monitoring

All patients are closely monitored for adverse events throughout the study, and adverse events are graded according to the National Cancer Institute Common Terminology Criteria for Adverse Events, Version 4.0 (NCI CTCAE v4.0). Patients undergo tumor assessments every 6 weeks (from Day 1 of Cycle 1) during the first 48 weeks and then every 6 or 12 weeks thereafter. Response is assessed by the investigator using RECIST v1.1. Response per modified RECIST v1.1 for immune based therapeutics (iRECIST) is determined programmatically on the basis of investigator-assessed individual lesion data. If clinical activity is demonstrated in an experimental arm, tumor assessment scans for that arm may be submitted for evaluation by an independent review facility.

Baseline tumor tissue samples are collected from all patients, e.g., by means of a biopsy performed at study entry. If a biopsy is not deemed feasible by the investigator, archival tumor tissue may be submitted after Medical Monitor approval has been obtained, provided the tissue was obtained within 3 months prior to enrollment and the patient has not received any anti-cancer therapy since the time of the biopsy. If deemed clinically feasible by the investigator, tumor tissue is collected for patients who discontinue Stage 1 because of unacceptable toxicity, disease progression per RECIST v1.1, or loss of clinical benefit as determined by the investigator. For patients enrolled in an experimental arm during the expansion phase, an on-treatment tumor tissue sample is collected 4 weeks after initiation of Stage 1 treatment (if clinically feasible), unless on-treatment tissue samples have already been collected, and determined to be evaluable, from a minimum of 15 patients treated with the same CIT combination. These samples are utilized for biomarker research (see rationale for biomarker assessments).

To characterize the pharmacokinetic (PK) properties and/or immunogenicity of atezolizumab and the other therapeutic agents, blood samples are taken at various timepoints before and during study treatment administration (Table 17). On the basis of a review of real-time safety data and available PK data, treatment regimens may be modified.

TABLE 17
Schedule of Pharmacokinetic, Immunogenicity, and Biomarker Samples

| Visit | Time | Sample Type |
|---|---|---|
| Day 1 of Cycle 1 | Prior to study treatment | Atezolizumab PK (serum) |
| | | Atezolizumab ADA (serum) |
| | | Tiragolumab PK (serum) |
| | | Tiragolumab ADA (serum) |
| | | Biomarker (plasma, serum, |
| | | PBMC) |
| | 30 minutes after atezolizumab | Atezolizumab PK (serum) |
| | infusion | |
| | 30 minutes after tiragolumab | Tiragolumab PK (serum) |
| | infusion | |
| Day 15 of Cycle 1 | Prior to study treatment | Biomarker (plasma, serum) |
| Day 1 of Cycle 2 | Prior to study treatment | Atezolizumab PK (serum) |
| | | Atezolizumab ADA (serum) |
| | | Tiragolumab PK (serum) |
| | | Tiragolumab ADA (serum) |
| | | Biomarker (plasma, serum, |
| | | PBMC) |
| Day 1 of Cycle 3 | Prior to study treatment | Atezolizumab PK (serum) |
| | | Atezolizumab ADA (serum) |
| | | Tiragolumab PK (serum) |
| | | Tiragolumab ADA (serum) |
| | 30 minutes after tiragolumab | Tiragolumab PK (serum) |
| | infusion | |
| Day 1 of Cycle 4 | Prior to study treatment | Atezolizumab PK (serum) |
| | | Atezolizumab ADA (serum) |
| | | Tiragolumab PK (serum) |
| | | Tiragolumab ADA (serum) |
| | | Biomarker (plasma, serum) |
| Day 1 of Cycle 8 | Prior to study treatment | Atezolizumab PK (serum) |
| | | Atezolizumab ADA (serum) |
| | | Tiragolumab PK (serum) |
| | | Tiragolumab ADA (serum) |
| | | Biomarker (plasma, serum) |
| Day 1 of Cycles 12 and 16 | Prior to study treatment | Atezolizumab PK (serum) |
| | | Atezolizumab ADA (serum) |
| | | Tiragolumab PK (serum) |
| | | Tiragolumab ADA (serum) |
| Treatment discontinuation visit | At visit | Atezolizumab PK (serum) |
| (≤30 days after last dose) | | Atezolizumab ADA (serum) |
| | | Tiragolumab PK (serum) |
| | | Tiragolumab ADA (serum) |
| | | Biomarker (plasma, serum) |

ADA = anti-drug antibody;
Atezo + Chemo + Tira = atezolizumab plus chemotherapy (nab-paclitaxel and gemcitabine) plus tiragolumab;
nab-paclitaxel = nanoparticle albumin-bound paclitaxel;
PBMC = peripheral blood mononuclear cell;
PK = pharmacokinetic.
Note:
On the basis of emerging safety or efficacy data, the number of PK and ADA samples may be reduced or sample collection may cease altogether. Additionally, collected samples may not be analyzed if not warranted. On the basis of emerging biomarker data, the number of biomarker samples may be reduced or sample collection may cease altogether.

### Laboratory, Biomarker, and Other Biological Samples

Exploratory biomarker research includes, but is not limited to, analysis of genes or gene signatures associated with tumor immunobiology, PD-L1, cytokines associated with T-cell activation, T-cell receptor repertoire, carcinoembryonic antigen, or density, localization, and activation status of immune cells and their subsets, and may involve DNA or RNA extraction, analysis of somatic mutations, and use of NGS (including WES).

Samples for the following laboratory tests are sent to the study site's local laboratory for analysis:
Hematology: WBC count, RBC count, hemoglobin, hematocrit, platelet count, and differential count (neutrophils, eosinophils, basophils, monocytes, lymphocytes, other cells)
Chemistry panel (serum or plasma): CPK, bicarbonate or carbon dioxide (if considered standard of care for the region), sodium, potassium, magnesium, chloride, glucose, BUN or urea, creatinine, total protein, albumin, phosphorus, calcium, total bilirubin, ALP, ALT, and AST
Coagulation: INR and aPTT
Thyroid function testing: thyroid-stimulating hormone, free triiodothyronine (T3) (or total T3 for sites where free T3 is not performed), and free thyroxine (also known as T4)
Ferritin and γ-glutamyl transferase
HIV serology, unless not permitted per local regulations
HAV serology: HAV IgM
HBV serology: HBsAg, total HBcAb, and (if HBsAg test is negative and total HBcAb test is positive) HBV DNA
   If a patient has a negative HBsAg test and a positive total HBcAb test at screening, an HBV DNA test must also be performed to determine if the patient has an HBV infection.
HCV serology: HCV antibody and (if HCV antibody test is positive) HCV RNA
   If a patient has a positive HCV antibody test at screening, an HCV RNA test must also be performed to determine if the patient has an active HCV infection.
HEV serology: HEV IgM
C-reactive protein
LDH
CA19-9
EBV serology:
   EBV VCA IgM
   EBV VCA IgG or EBNA IgG
   EBV PCR (only if clinically indicated)
Pregnancy test
   All women of childbearing potential have a serum pregnancy test at Stage 1 screening. Urine or serum pregnancy tests are performed at specified subsequent visits. If a urine pregnancy test is positive, it must be confirmed by a serum pregnancy test.
   A woman is considered to be of childbearing potential if she is postmenarcheal, has not reached a postmenopausal state (12 continuous months of amenorrhea with no identified cause other than menopause), and has not undergone surgical sterilization (removal of ovaries and/or uterus).
Urinalysis (pH, specific gravity, glucose, protein, ketones, and blood)
   Dipstick urinalysis is permitted. However, patients with ≥2+ protein on dipstick urinalysis at screening must undergo a 24-hour urine collection for protein if a bevacizumab-containing arm is open for enrollment.

Samples for the following laboratory test are sent to a central laboratory or to the study site's local laboratory for analysis:
Soluble CD25

The following samples are sent to one or several central laboratories or to the Sponsor for analysis:
Serum sample for analysis of autoantibodies: anti-nuclear antibody, anti-double-stranded DNA, circulating anti-neutrophil cytoplasmic antibody, and perinuclear anti-neutrophil cytoplasmic antibody
Plasma or serum samples for PK analysis through use of validated assays
Plasma or serum samples for immunogenicity analysis through use of validated assays
Plasma, serum, and peripheral blood mononuclear cell (PBMC) samples for exploratory research on biomarkers
Tumor tissue sample collected at baseline for determination of PD-L1 expression and for exploratory research on biomarkers
   Baseline tumor tissue samples from the primary lesion or a metastatic lesion are collected from all patients, preferably by means of a biopsy performed at study entry. If a biopsy is not deemed feasible by the investigator, archival tumor tissue may be submitted after Medical Monitor approval has been obtained, provided the tissue was obtained from a biopsy performed within 3 months prior to enrollment and the patient has not received any anti-cancer therapy since the time of the biopsy.
   A representative FFPE tumor specimen in a paraffin block (preferred) or at least 16 slides containing unstained, freshly cut, serial sections must be submitted along with an associated pathology report. If only 10-15 slides are available, the patient may still be eligible for the study, after Medical Monitor approval has been obtained.
   Tumor tissue should be of good quality based on total and viable tumor content. Samples must contain a minimum of 50 viable tumor cells that preserve cellular context and tissue architecture regardless of needle gauge or retrieval method. Samples collected via resection, core-needle biopsy (at least three cores, 18-gauge needle or larger [16-gauge needle preferred], embedded in a single paraffin block), or excisional, incisional, punch, or forceps biopsy are acceptable. Fine-needle aspiration (defined as samples that do not preserve tissue architecture and yield cell suspension and/or smears), brushing, cell pellets from pleural effusion, and lavage samples are not acceptable. Tumor tissue from bone metastases that have been decalcified is not acceptable.
   Remaining archival tumor tissue blocks are returned to the site upon request or 18 months after final closure of the study database, whichever occurs first.
Patients enrolled in an experimental arm during the expansion phase: tumor tissue sample collected 4 weeks (±7 days) after initiation of treatment (if deemed clinically feasible by the investigator) for exploratory research on biomarkers
   Samples are not collected if on-treatment tissue samples have already been collected, and determined to be evaluable, from a minimum of 15 patients treated with the same CIT combination.
   Samples collected via resection, core-needle biopsy (at least three cores preferred), or excisional, incisional, punch, or forceps biopsy are preferred.
Tumor tissue sample collected at the time of unacceptable toxicity, disease progression per RECIST v1.1, or loss of clinical benefit as determined by the investigator, if deemed clinically feasible by the investigator, for exploratory research on biomarkers
   Biopsies should be performed within 40 days after determination of unacceptable toxicity, disease progression, or loss of clinical benefit, or prior to the next anti-cancer therapy, whichever is sooner. Samples collected via resection, core-needle biopsy (at least three cores preferred), or excisional, incisional, punch, or forceps biopsy are preferred.

### Atezolizumab Dose and Schedule

Atezolizumab is administered at a fixed dose of 840 mg every two weeks (Q2W) (840 mg on Days 1 and 15 of each 28-day cycle).

### Tiragolumab Dose and Schedule

Tiragolumab is administered at a fixed dose of 420 mg Q2W (420 mg on Days 1 and 15 of each 28-day cycle). The average concentration following the 420 mg Q2W dose is expected to be equivalent to that of 600 mg every three weeks (Q3W). The fixed tiragolumab dose of 600 mg IV Q3W was selected on the basis of available pharmacokinetic (PK), efficacy, and safety data from Study GO30103, in which patients received single-agent tiragolumab or tiragolumab plus atezolizumab. The MTD was not reached, and no DLTs were observed with tiragolumab monotherapy or with tiragolumab at doses of 2-1200 mg Q3W in combination with atezolizumab 1200 mg Q3W. In addition, development of anti-drug antibodies (ADAs) to tiragolumab was observed in 3 of 145 evaluable patients receiving tiragolumab (doses of 2-600 mg Q3W) in combination with atezolizumab. Complete occupancy of peripheral TIGIT receptors on CD4+, CD8+, and NK cells was observed beginning at the 30 mg Q3W dose of tiragolumab and remained sustained at all higher doses. Anti-tumor activity (radiographic partial response) was observed at tiragolumab doses of 30-600 mg Q3W when given in combination with atezolizumab 1200 mg Q3W.

### Dosage and Administration

Patients receive treatment as outlined until unacceptable toxicity or loss of clinical benefit as determined by the investigator after an integrated assessment of radiographic and biochemical data, local biopsy results (if available), and clinical status (e.g., symptomatic deterioration such as pain secondary to disease).

Participants receive atezolizumab 840 mg IV infusion on Days 1 and 15 of each 28 day cycle per the instructions outlined in Table 18.

Participants receive tiragolumab 420 mg IV infusion on Days 1 and 15 of each 28 day cycle per the instructions outlined in Table 19. On Day 1 of Cycle 1, tiragolumab is administered 60 minutes after completion of the atezolizumab infusion. The interval between subsequent infusions is 30 minutes if the previous atezolizumab infusion was given without premedication and tolerated without an infusion-related reaction (IRR) or 60 minutes if the patient experienced an IRR with the previous atezolizumab infusion.

Participants receive nab-paclitaxel 125 mg/m² IV infusion on Days 1, 8, and 15 of each 28 day cycle, administered by IV infusion over 30 (±5) minutes, followed by gemcitabine 1000 mg/m², administered by IV infusion over 30 (±10) minutes. On Day 1 of Cycle 1, nab-paclitaxel is administered 60 minutes after completion of the tiragolumab infusion. The interval between subsequent infusions is 30 minutes if the previous tiragolumab infusion was tolerated without an IRR or 60 minutes if the patient experienced an IRR with the previous tiragolumab infusion.

TABLE 18
Administration of First and Subsequent Atezolizumab Infusions

| First Infusion | Subsequent Infusions |
|---|---|
| No premedication is permitted prior to the | If the patient experienced an infusion-related |
| atezolizumab infusion, | reaction with any previous infusion, premedication |
| Vital signs (pulse rate, respiratory rate, blood | with antihistamines, anti-pyretics, and/or |
| pressure, pulse oximetry, and temperature) are | analgesics may be administered for subsequent |
| recorded within 60 minutes prior to the infusion, | doses at the discretion of the investigator. |
| Atezolizumab is infused over 60 (±15) minutes. | Vital signs are recorded within 60 minutes prior |
| If clinically indicated, vital signs are measured | to the infusion. |
| every 15 (±5) minutes during the infusion and at | Atezolizumab is infused over 30 (±10) minutes if |
| 30 (±10) minutes after the infusion, | the previous infusion was tolerated without an |
| Patients are informed about the possibility of | infusion-related reaction, or 60 (±15) minutes if |
| delayed post-infusion symptoms and instructed to | the patient experienced an infusion-related |
| contact their study physician if they develop such | reaction with the previous infusion. |
| symptoms. | If the patient experienced an infusion-related |
| | reaction with the previous infusion or if clinically |
| | indicated, vital signs should be measured during |
| | the infusion and at 30 (±10) minutes after the |
| | infusion. |

TABLE 19
Administration of First and Subsequent Tiragolumab Infusions

| First Infusion | Subsequent Infusions |
|---|---|
| No premedication is permitted prior to the | If the patient experienced an infusion-related |
| tiragolumab infusion, | reaction with any previous infusion, premedication |
| Vital signs (pulse rate, respiratory rate, blood | with antihistamines, anti-pyretics, and/or |
| pressure, pulse oximetry, and temperature) are | analgesics may be administered for subsequent |
| recorded within 60 minutes prior to the infusion, | doses at the discretion of the investigator. |
| Tiragolumab is infused over 60 (±10) minutes. | Vital signs are recorded within 60 minutes prior |
| Vital signs are recorded every 15 (±5) minutes | to the infusion. |
| during the infusion and at 30 (+10) minutes after | Tiragolumab is infused over 30 (±10) minutes if |
| the infusion, | the previous infusion was tolerated without an |
| Patients are observed for 60 minutes after | infusion-related reaction, or 60 (±10) minutes if |
| completion of the tiragolumab infusion, | the patient experienced an infusion-related |
| Patients are informed about the possibility of | reaction with the previous infusion. |
| delayed post-infusion symptoms and are | Patients are observed for 30 minutes after |
| instructed to contact their study physician if they | completion of the tiragolumab infusion if the |
| develop such symptoms. | previous infusion was tolerated without an |
| | infusion-related reaction, or 60 minutes after |
| | completion of the tiragolumab infusion if the |
| | patient experienced an infusion-related reaction |
| | with the previous infusion. |
| | If the patient experienced an infusion-related |
| | reaction with the previous infusion or if clinically |
| | indicated, vital signs should be recorded during |
| | the infusion and at 15 (±10) minutes after the |
| | infusion. |

### Dose Modifications

There are no dose modifications for atezolizumab or tiragolumab in this study. For management of drug-related toxicities, the dose of nab-paclitaxel may be reduced by 25 mg/m² (one dose level) up to two times and the dose of gemcitabine may be reduced by 200 mg/m² (one dose level) up to two times, as outlined in Table 20.

If further dose reduction is indicated for nab-paclitaxel and/or gemcitabine after two dose reductions, that drug (or both drugs, if applicable) is discontinued, but the patient may continue other study treatments at the investigator's discretion. After dose reduction, the dose may be escalated during subsequent administrations at the investigator's discretion.

TABLE 20
Recommended Dose Reductions for Nab-Paclitaxel
and Gemcitabine

| | | First Dose | Second Dose |
|---|---|---|---|
| | Initial Dose | Reduction | Reduction |
| Nab-paclitaxel | 125 mg/m² | 100 mg/m² | 75 mg/m² |
| Gemcitabine | 1000 mg/m² | 800 mg/m² | 600 mg/m² |

### Concomitant Therapy

Concomitant therapy includes of any medication (e.g., prescription drugs, over-the-counter drugs, vaccines, herbal or homeopathic remedies, nutritional supplements) used by a patient in addition to protocol-mandated study treatment from 10 days prior to initiation of study treatment to the treatment discontinuation visit.

Patients are permitted to use the following therapies during the study:
Colony-stimulating factors (CSFs), such as granulocyte colony-stimulating factors (G-CSFs), and erythropoiesis-stimulating agents (ESAs) per local practice/institutional guidelines or the American Society of Clinical Oncology guidelines for hematopoietic CSFs (Smith et al. 2006) and American Society of Hematology/American Society of Clinical Oncology guidelines for ESAs (Rizzo et al. 2010)
   Evidence supporting the use of long-acting (PEGylated) forms of G-CSF in patients receiving weekly chemotherapy (i.e., nab-paclitaxel) is limited. Thus, investigators should consider giving preference to conventional formulations of G-CSF.
Oral contraceptives
Hormone-replacement therapy
Prophylactic or therapeutic anticoagulation therapy (such as warfarin at a stable dose or low-molecular-weight heparin)
Inactivated influenza vaccinations
Megestrol acetate administered as an appetite stimulant after initiation of study treatment
Mineralocorticoids (e.g., fludrocortisone)
Inhaled corticosteroids administered for chronic obstructive pulmonary disease or asthma
Low-dose corticosteroids administered for orthostatic hypotension or adrenocortical insufficiency
Hormonal therapy with gonadotropin-releasing hormone agonists or antagonists for prostate cancer
   Palliative radiotherapy (e.g., treatment of known bony metastases or symptomatic relief of pain) as outlined below: Palliative radiotherapy is permitted, provided it does not interfere with the assessment of tumor target lesions (e.g., the lesion to be irradiated must not be the only site of measurable disease). Treatment with nab-paclitaxel and gemcitabine should be withheld during palliative radiotherapy. Treatment with atezolizumab and tiragolumab may be continued during palliative radiotherapy.
Radiotherapy to the brain as outlined below:
   Patients whose extracranial tumor burden is stable or responding to study treatment and who are subsequently found to have three or fewer brain metastases may receive radiotherapy to the brain (either stereotactic radiosurgery or whole-brain radiation therapy) provided that all of the following criteria are met:
      The patient has no evidence of progression or hemorrhage after completion of CNS-directed therapy.
      The patient has no ongoing requirement for corticosteroids as therapy for CNS disease.
         Patients who require corticosteroid therapy for more than 7 days after completion of radiotherapy must be discontinued from study treatment.
      Anti-convulsant therapy, if required, is administered at a stable dose.

Premedication with antihistamines, anti-pyretics, and/or analgesics may be administered for the second and subsequent atezolizumab and tiragolumab infusions only, at the discretion of the investigator. In general, investigators should manage a patient's care with supportive therapies as clinically indicated, per local standard practice. Patients who experience infusion associated symptoms may be treated symptomatically with acetaminophen, ibuprofen, diphenhydramine, and/or H2-receptor antagonists (e.g., famotidine, cimetidine), or equivalent medications per local standard practice. Serious infusion-associated events manifested by dyspnea, hypotension, wheezing, bronchospasm, tachycardia, reduced oxygen saturation, or respiratory distress should be managed with supportive therapies as clinically indicated (e.g., supplemental oxygen and 32-adrenergic agonists).

### Inclusion Criteria

Patients meet the following criteria:
Age ≥18 years at the time of signing Informed Consent Form
ECOG Performance Status of 0 or 1
Histologically or cytologically confirmed metastatic PDAC
   The definitive diagnosis of metastatic PDAC is made by evaluating the histopathologic data within the context of clinical and radiographic data.
   Patients with endocrine or acinar pancreatic carcinoma are not eligible for the study.
No prior systemic treatment for PDAC
Life expectancy ≥3 months, as determined by the investigator
Availability of a representative tumor specimen that is suitable for determination of PD-L1 and/or additional biomarker status via central testing
   Baseline tumor tissue samples are collected from all patients, preferably by means of a biopsy performed at study entry. If a biopsy is not deemed feasible by the investigator, archival tumor tissue may be submitted after Medical Monitor approval has been obtained, provided the tissue was obtained from a biopsy performed within 3 months prior to enrollment and the patient has not received any anti-cancer therapy since the time of the biopsy.
   A formalin-fixed, paraffin-embedded tumor specimen in a paraffin block (preferred) or at least 16 slides containing unstained, freshly cut, serial sections must be submitted along with an associated pathology report prior to study enrollment. If only 10-15 slides are available, the patient may still be eligible for the study, after Medical Monitor approval has been obtained.
Signed Informed Consent Form
Ability to comply with the study protocol, in the investigator's judgment
Measurable disease (at least one target lesion) according to RECIST v1.1
   Previously irradiated lesions can be considered as measurable disease only if progressive disease has been unequivocally documented at that site since radiation.
Adequate hematologic and end-organ function, defined by the following laboratory test results, obtained within 14 days prior to initiation of study treatment:
   ANC ≥1.5×10⁹/L (1500/μL) without granulocyte colony-stimulating factor support within 14 days prior to screening laboratory test
   WBC count ≥2.5×10⁹/L (2500/μL)
   Lymphocyte count ≥0.5×10⁹/L (500/μL)
Platelet count ≥100×10⁹/L (100,000/μL) without transfusion within 7 days prior to screening laboratory test
Hemoglobin ≥90 g/L (9.0 g/dL)
   Patients may be transfused to meet this criterion after discussion with the Medical Monitor.
AST, ALT, and ALP ≤2.5× upper limit of normal (ULN), with the following exceptions:
   Patients with documented liver metastases: AST and ALT ≤5×ULN
   Patients with documented liver or bone metastases: ALP ≤5×ULN
Serum bilirubin ≤1.5×ULN with the following exception:
   Patients with known Gilbert disease: serum bilirubin level ≤3×ULN
   Creatinine clearance ≥50 mL/min (calculated using the Cockcroft-Gault formula)
   Serum albumin ≥25 g/L (2.5 g/dL)
   For patients not receiving anticoagulation: INR or aPTT ≤1.5×ULN
For patients receiving therapeutic anticoagulation: stable anticoagulant regimen
Tumor accessible for biopsy
For women of childbearing potential: agreement to remain abstinent (refrain from heterosexual intercourse) or use contraceptive measures, and agreement to refrain from donating eggs
For men: agreement to remain abstinent (refrain from heterosexual intercourse) or use contraceptive measures, and agreement to refrain from donating sperm

### Safety

Measures are taken to ensure the safety of patients participating in this study, including the use of stringent inclusion and exclusion criteria and close monitoring of patients during the study. Administration of study treatment is performed in a monitored setting in which there is immediate access to trained personnel and adequate equipment and medicine to manage potentially serious reactions. Adverse events are reported as described.

Verbatim adverse event terms are mapped to Medical Dictionary for Regulatory Activities thesaurus terms, and adverse event severity is graded according to NCI CTCAE v4.0.

Safety is assessed through summaries of adverse events, changes in laboratory test results, changes in vital signs and ECGs, and exposure to study drugs. Exposure to combination treatment and length of safety follow-up are summarized by treatment arm within each stage.

Treatment-emergent adverse events occurring after initiation of treatment are summarized. For each patient, the maximum reported severity of each adverse event is used in the summaries by severity grade. All treatment-emergent adverse events, serious adverse events, adverse events leading to withdrawal of study treatment, Grade 3 adverse events, deaths, and causes of death are listed and summarized by mapped term, appropriate thesaurus level, and NCI CTCAE severity grade. Relevant laboratory, vital sign (pulse rate, respiratory rate, blood pressure, pulse oximetry, and temperature), and ECG data are displayed by time, with grades identified where appropriate. Additionally, a shift table of selected laboratory tests is used to summarize the baseline and maximum post-baseline severity grade. Changes in vital signs and ECGs are summarized.

Atezolizumab has been associated with risks such as the following: IRRs and immune-mediated hepatitis, pneumonitis, colitis, pancreatitis, diabetes mellitus, hypothyroidism, hyperthyroidism, adrenal insufficiency, hypophysitis, Guillain-Barré syndrome, myasthenic syndrome or myasthenia gravis, meningoencephalitis, myocarditis, nephritis, and myositis. Immune-mediated reactions may involve any organ system and may lead to hemophagocytic lymphohistiocytosis and macrophage activation syndrome (considered to be potential risks for atezolizumab).

The following are the most common adverse events observed with nab-paclitaxel in patients with PDAC: neutropenia, fatigue, peripheral neuropathy, nausea, alopecia, peripheral edema, diarrhea, pyrexia, vomiting, decreased appetite, rash, and dehydration. The following adverse events have also been observed: myelosuppression (primarily neutropenia, anemia, thrombocytopenia), cranial nerve palsies, hypersensitivity reactions, pneumonitis, myalgia, arthralgia, cardiotoxicity (myocardial disorders, cardiac failure, angina, tachycardia, ventricular arrhythmia), cystoid macular edema, Stevens-Johnson syndrome/toxic epidermal necrolysis, sepsis, infusion-site reactions/extravasation, hepatic toxicity (drug-induced liver injury), acute renal failure, hemolytic-uremic syndrome, and drug-induced lupus erythematous.

The most common adverse events observed with gemcitabine are nausea/vomiting, anemia, hepatic transaminitis, neutropenia, increased ALP, proteinuria, fever, hematuria, rash, thrombocytopenia, dyspnea, and peripheral edema.

IRR is an identified risk for tiragolumab. While clinical evaluation of tiragolumab is limited and not all risks are known, as an antagonist of TIGIT, tiragolumab is anticipated to enhance T-cell and NK-cell proliferation, survival, and function. Therefore, tiragolumab may increase the risk of autoimmune inflammation (also described as immune-mediated adverse events). In addition, due to the intact Fc effector function of tiragolumab, lymphopenia via antibody-dependent cellular cytotoxicity (ADCC) is a theoretical risk.

Because tiragolumab is a therapeutic monoclonal antibody and targets immune cells, IRRs associated with hypersensitivity reactions, target-mediated cytokine release, and/or emergent ADAs may occur. Clinical signs and symptoms of such reactions may include rigors, chills, wheezing, pruritus, flushing, rash, hypotension, hypoxemia, and fever. IRRs have been reported in patients treated with tiragolumab alone or in combination atezolizumab. The majority of events were mild to moderate and manageable.

To minimize the risk and sequelae of IRRs, the initial dose of tiragolumab is administered over 60 minutes followed by a 60-minute observation period. Subsequent infusions and observation times may be shortened if the preceding infusion was well tolerated. All infusions of tiragolumab are administered in an appropriate medical setting.

Nonclinical models have suggested a role of TIGIT signaling interruption in autoimmunity. In a knockout model (TIGIT −/−), loss of TIGIT signaling resulted in hyperproliferative T-cell responses and exacerbation of experimental autoimmune encephalitis (EAE). TIGIT −/− and wild-type B6 mice were immunized with suboptimal doses of myelin oligodendrocyte glycoprotein peptide to induce EAE. In contrast to the wild-type B6 mice, the majority of the TIGIT −/− mice developed severe EAE (Joller et al. 2011).

Clinical experience with therapeutics intended to enhance anti-tumor T-cell responses has demonstrated that development of autoimmune inflammatory conditions is a general risk and may therefore be considered a potential risk of tiragolumab. Such immune-mediated adverse events have been described for virtually all organ systems and include, but are not limited to, colitis, hepatitis, pneumonitis, endocrinopathy, ocular toxicity, pancreatic toxicity, neurologic toxicity, myocarditis, nephritis, myositis, and rash.

Patients with a history of autoimmune disease are excluded from this study. In addition, patients with a history of severe immune-mediated adverse events associated with prior immunotherapy or adverse events that did not resolve to baseline after discontinuation of prior immunotherapy are excluded from this study.

In this study, specified immune-mediated adverse events are considered adverse events of special interest and are captured accordingly.

Given the IgG1 backbone of tiragolumab with intact Fc-effector function, ADCC-mediated reduction in lymphocyte count is a potential risk. However, in a repeat-dose toxicity study in cynomolgus monkeys, there were no tiragolumab-related decreases in overall lymphocyte counts.

Transient lymphocyte count decreases without clinical sequelae have been observed in patients treated with tiragolumab, alone or in combination with atezolizumab, in the Phase I study in solid tumors (Study GO30103). Because of this potential risk of tiragolumab to induce lymphopenia, patients with a lymphocyte count <0.5×10⁹/L (500/μL) are excluded from the study. Complete blood counts are monitored throughout the study.

The following adverse events are potential overlapping toxicities associated with combination use of atezolizumab, nab-paclitaxel, gemcitabine, and tiragolumab: immune-mediated toxicities, including hemophagocytic lymphohistiocytosis, macrophage activation syndrome, and others, gastrointestinal toxicities, hematologic toxicity, and dermatologic toxicities.

On Day 1 of each cycle, patients are required to have an ANC of ≥1.5×10⁹/L (1500/μL) and a platelet count of ≥100×10⁹/L (100,000/μL) to receive treatment with nab-paclitaxel and gemcitabine.

### Treatment Interruption for Toxicities

Atezolizumab and/or tiragolumab may be temporarily suspended in patients experiencing toxicity considered to be related to study treatment. If corticosteroids are initiated for treatment of the toxicity, they must be tapered over ≥1 month to equivalent of ≤10 mg/day oral prednisone or equivalent before drug can be resumed. If atezolizumab or tiragolumab is withheld for >12 weeks, the patient is discontinued from that drug. However, the drug may be withheld for >12 weeks to allow for patients to taper off corticosteroids prior to resuming treatment. Atezolizumab or tiragolumab can be resumed after being withheld for >12 weeks if the Medical Monitor agrees that the patient is likely to derive clinical benefit.

On the basis of the available characterization of mechanism of action, tiragolumab may cause adverse events similar to, but independent of, atezolizumab. Tiragolumab may also exacerbate the frequency or severity of atezolizumab-related adverse events or may have non-overlapping toxicities with atezolizumab. Because these scenarios may not be distinguishable from each other in the clinical setting, immune-mediated adverse events should generally be attributed to both agents, and dose interruptions or treatment discontinuation in response to immune-mediated adverse events should be applied to both tiragolumab and atezolizumab.

Nab-paclitaxel and/or gemcitabine treatment may be temporarily suspended in patients experiencing toxicity considered to be related to study treatment. If nab-paclitaxel or gemcitabine have been withheld for >56 days because of toxicity, the patient should be discontinued from both chemotherapy agents. However, nab-paclitaxel or gemcitabine can be resumed after being withheld for >56 days if the Medical Monitor agrees that the patient is likely to derive clinical benefit.

If atezolizumab is discontinued, tiragolumab should also be discontinued, but nab-paclitaxel and gemcitabine may be continued if the patient is likely to derive clinical benefit, as determined by the investigator. If nab-paclitaxel, gemcitabine, or tiragolumab is discontinued, the other drugs can be continued if the patient is likely to derive clinical benefit, as determined by the investigator.

### Statistics

The final study analysis is based on patient data collected through study discontinuation. If not otherwise specified, efficacy analyses are based on the efficacy-evaluable population, defined as all patients who receive at least one dose of each drug for their assigned treatment regimen, and safety analyses are based on the safety-evaluable population, defined as all patients who receive any amount of study treatment.

Data are described and summarized as warranted by sample size. Continuous variables are summarized through use of means, standard deviations, medians, and ranges. Categorical variables are summarized through use of counts and percentages. Listings are used in place of tables in the event of small sample sizes.

This study is not designed to make explicit power and Type I error considerations for a hypothesis test. Instead, this study is designed to obtain preliminary efficacy, safety, and PK data on immunotherapy-based treatment combinations when administered to patients with metastatic PDAC.

Table 21 shows estimated differences in ORR between an experimental arm and a control arm, along with 90% confidence intervals, with a sample size of 15 patients each in the preliminary phase, assuming asymptotic normality.

TABLE 21
Estimated Differences in Objective Response Rate
between Experimental and Control Arms of
15 Patients Each (Preliminary Phase)

| | | Difference |
|---|---|---|
| | | (Percentage Points) in |
| Experimental | Control | Objective Response |
| Arm (n = 15) | Arm (n = 15) | Rate (90% Cl)^{a} |
| 7 (46.7%) | 4 (26.7%) | 20% (−8%, 48%) |
| 8 (53.3%) | 4 (26.7%) | 26.7% (−2%, 55%) |
| 9 (60.0%) | 5 (33.3%) | 26.7% (−2%, 56%) |

Cl = confidence interval.
^{a}Asymptotic confidence limits (not corrected for continuity as the sample size is very small).

Table 22 shows estimated differences in ORR between an experimental arm and a control arm, along with 90% confidence intervals, with a sample size of 40 patients each in the preliminary and expansion phases combined, assuming asymptotic normality.

TABLE 22
Estimated Differences in Objective Response
Rate between Experimental and Control Arms of
15 Patients Each (Preliminary Phase)

| | | Difference |
|---|---|---|
| | | (Percentage Points) in |
| Experimental | Control | Objective Response |
| Arm (n = 40) | Arm (n = 40) | Rate (90% Cl)^{a} |
| 16 (40%) | 10 (25%) | 15% (−5%, 35%) |
| 18 (45%) | 10 (25%) | 20% (0%, 40%) |
| 20 (50%) | 12 (30%) | 20% (0%, 40%) |
| 22 (55%) | 12 (30%) | 25% (5%, 45%) |

Cl = confidence interval.
^{a}Asymptotic confidence limits (not corrected for continuity as the sample size is very small).

### Objectives and Endpoints

A summary of objectives and corresponding endpoints for the study can be found in Table 23.

TABLE 23
Objectives and Corresponding Endpoints

| Objective | Corresponding Endpoint |
|---|---|
| Primary Efficacy Objective: | Objective response, defined as a complete |
| To evaluate the efficacy of immunotherapy- | response or partial response on two |
| based treatment combinations | consecutive occasions ≥4 weeks apart, as |
| | determined by the investigator according to |
| | RECIST v1.1 |
| Secondary Efficacy Objective: | PFS after randomization, defined as the time |
| To evaluate the efficacy of immunotherapy- | from randomization to the first occurrence of |
| based treatment combinations | disease progression or death from any cause |
| | (whichever occurs first), as determined by the |
| | investigator according to RECIST v1.1 |
| | OS after randomization, defined as the time |
| | from randomization to death from any cause |
| | OS at specific timepoints (e.g., 6 months) |
| | DOR, defined as the time from the first |
| | occurrence of a documented objective |
| | response to disease progression or death from |
| | any cause (whichever occurs first), as |
| | determined by the investigator according to |
| | RECIST v1.1 |
| | Disease control, defined as stable disease for |
| | ≥12 weeks or a complete or partial response, |
| | as determined by the investigator according to |
| | RECIST v1.1 |
| Exploratory Efficacy Objective: | Objective response, as determined by the |
| To evaluate the efficacy of immunotherapy- | investigator according to iRECIST |
| based treatment combinations | PFS after randomization, as determined by the |
| | investigator according to iRECIST DOR, as |
| | determined by the investigator according to |
| | iRECIST |
| | Disease control, as determined by the |
| | investigator according to iRECIST |
| | Change from baseline in CA19-9 at |
| | subsequent timepoints |
| Safety Objective: | Incidence, nature, and severity of adverse |
| To evaluate the safety of immunotherapy- | events and laboratory abnormalities, with |
| based treatment combinations | severity determined according to National |
| | Cancer Institute Common Terminology Criteria |
| | for Adverse Events, Version 4.0 |
| | Change from baseline in vital signs and ECG |
| | parameters |
| | Change from baseline in targeted clinical |
| | laboratory test results |
| Exploratory Pharmacokinetic Objectives: | Plasma or serum concentration of each drug |
| To characterize the PK profile of drugs that are | (as appropriate) at specified timepoints |
| administered as part of an immunotherapy- | Relationship between plasma or serum |
| based treatment combination | concentration or PK parameters for each drug |
| To evaluate potential relationships between | (as appropriate on the basis of available data) |
| drug exposure and the efficacy and safety of | and efficacy endpoints |
| immunotherapy-based treatment combinations | Relationship between plasma or serum |
| | concentration or PK parameters for each drug |
| | (as appropriate on the basis of available data) |
| | and safety endpoints |
| Exploratory Immunogenicity Objectives: | For drugs for which ADA formation is |
| To evaluate the immune response to drugs | measured: presence of ADAs during the study |
| that are administered as part of an | relative to the presence of ADAs at baseline |
| immunotherapy-based treatment combination | For drugs for which ADA formation is |
| To evaluate potential effects of ADAs | measured: relationship between ADA status |
| | and efficacy, safety, or PK endpoints |
| Exploratory Biomarker Objective: | Relationship between biomarkers in blood and |
| To identify biomarkers that are predictive of | tumor tissue and efficacy, safety, PK, |
| response to study treatment (i.e., predictive | immunogenicity, or other biomarker endpoints |
| biomarkers), are associated with progression | |
| to a more severe disease state (i.e., prognostic | |
| biomarkers), are associated with resistance to | |
| study treatment, are associated with | |
| susceptibility to developing adverse events | |
| (i.e., safety biomarkers), can provide evidence | |
| of study treatment activity (i.e., | |
| pharmacodynamic biomarkers), or can | |
| increase the knowledge and understanding of | |
| disease biology | |

ADA = anti-drug antibody;
DOR = duration of response;
iRECIST = modified RECIST v1.1 for immune-based therapeutics;
OS = overall survival;
PFS = progression-free survival;
PK = pharmacokinetic;
RECIST = Response Evaluation Criteria in Solid Tumors.
Overall response at a single timepoint is assessed by the investigator using RECIST v1.1. Overall response per iRECIST is not captured in the eCRF, but is calculated programmatically on the basis of investigator-assessed individual lesion data recorded in the eCRF.

### Efficacy Analysis

The primary efficacy endpoint is objective response. ORR, the proportion of patients with a complete or partial response, is calculated for each arm, along with 90% confidence intervals (Clopper-Pearson method). The difference in ORR between the experimental arms and the control arm is calculated, along with 90% confidence intervals. Confidence intervals are estimated by asymptotic normality methods, depending on the sample size.

The secondary efficacy endpoints are PFS, OS, OS at specific timepoints (e.g., 6 months), duration of response (DOR), and disease control PFS, DOR, and disease control are determined by the investigator according to RECIST v1.1. DOR is derived for efficacy-evaluable patients with a complete or partial response. For patients who do not have documented disease progression or death in a study stage, PFS and DOR are censored at the day of the last tumor assessment. Patients who are still alive at the time of OS analysis are censored at the last date they were known to be alive.

The Kaplan-Meier method is used to estimate the median for PFS, OS, and DOR, with 90% confidence intervals constructed through use of the Brookmeyer and Crowley method. OS rate at specific timepoints are estimated using the Kaplan-Meier method, with 90% confidence intervals calculated on the basis of Greenwood's estimate for the variance. Disease control rate, the proportion of patients with stable disease for ≥12 weeks, a partial response, or a complete response, is calculated for each treatment arm, with 90% confidence intervals estimated through use of Clopper-Pearson's exact method.

The exploratory efficacy endpoints are objective response, PFS, DOR, and disease control as determined by the investigator according to iRECIST; and change from baseline in CA19-9 at subsequent timepoints during both stages. DOR is derived for efficacy-evaluable patients with a complete or partial response. CA19-9 change from baseline over time is summarized. In addition, the proportion of patients with a maximum decrease from baseline in CA19-9 of ≥50% or other thresholds may be calculated for each treatment arm, with 90% confidence intervals estimated through use of Clopper-Pearson's exact method.

### Pharmacokinetic Analyses

Sparse samples can be collected for potential PK analyses of atezolizumab (patients who receive at least one dose of atezolizumab) and specified drugs given in combination with atezolizumab (patients who receive at least one dose of the drug). Serum or plasma concentrations of the various study drugs may be reported as individual values and summarized (mean, standard deviation, coefficient of variation, median, range, geometric mean, and geometric mean coefficient of variation) by treatment arm, and by cycle and day when appropriate and as data allow. Individual and median serum or plasma concentrations of the various study drugs may be plotted by treatment arm and cycle and day. PK data for combination drugs may be compared with available historical data from internal and published previous studies. Atezolizumab concentration data may be pooled with data from other studies using an established population PK model to derive PK parameters such as clearance, volume of distribution, and area under the curve.

### Immunogenicity Analysis

Immunogenicity may be assessed for atezolizumab and other study treatments as appropriate (refer to arm-specific appendices for details). The immunogenicity analyses include all patients with at least one anti-drug antibody (ADA) assessment. Patients are grouped according to treatment received or, if no treatment is received prior to study discontinuation, according to treatment assigned.

For atezolizumab, the numbers and proportions of ADA-positive patients and ADA-negative patients at baseline (baseline prevalence) and after baseline (post-baseline incidence) are summarized by treatment group. When determining post-baseline incidence, patients are considered to be ADA positive if they are ADA negative or are missing data at baseline but develop an ADA response following study drug exposure (treatment-induced ADA response), or if they are ADA positive at baseline and the titer of one or more post-baseline samples is at least 0.60 titer units greater than the titer of the baseline sample (treatment-enhanced ADA response). Patients are considered to be ADA negative if they are ADA negative or are missing data at baseline and all post-baseline samples are negative, or if they are ADA positive at baseline but do not have any post-baseline samples with a titer that is at least 0.60 titer units greater than the titer of the baseline sample (treatment unaffected).

For other study treatments for which ADAs are tested, ADA positivity is determined according to standard methods established for previous studies of these drugs. The relationship between ADA status and safety, efficacy, PK, and biomarker endpoints may be analyzed and reported via descriptive statistics.

### Example 3. A Phase Ib, Open-Label, Multicohort Study of the Safety, Efficacy, and Pharmacokinetics of Tiragolumab in Combination with Atezolizumab and Chemotherapy in Patients with Triple-Negative Breast Cancer

The present example describes a Phase Ib, open-label, multicohort study designed to evaluate the safety, efficacy, and pharmacokinetics of tiragolumab in combination with atezolizumab and chemotherapy in patients with metastatic triple-negative breast cancer (TNBC). The study consists of the following cohort:
Cohort A enrolls patients with unresectable, locally advanced, or metastatic programmed death-ligand 1 (PD-L1)-positive TNBC (first-line metastatic TNBC) who have not received prior systemic therapy for metastatic breast cancer. Patients receive tiragolumab in combination with atezolizumab and nab-paclitaxel.

A. Study Design

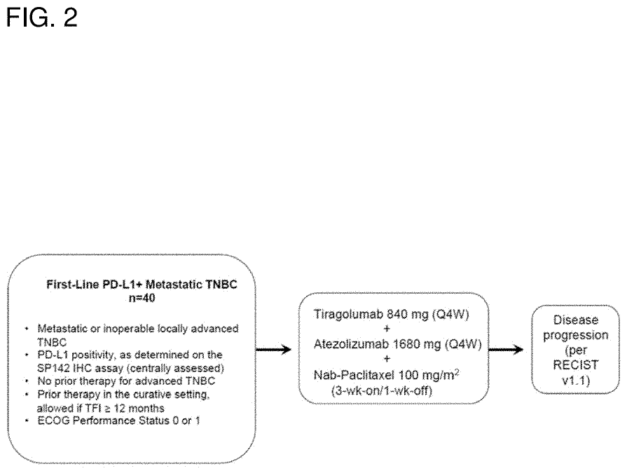

This Phase Ib, multicohort, open-label, multicenter, global study is designed to investigate the safety and tolerability, preliminary efficacy, and pharmacokinetics of tiragolumab in combination with atezolizumab and nab-paclitaxel in patients with unresectable locally advanced or metastatic PD-L1-positive TNBC who have not received prior systemic therapy for metastatic breast cancer (referred to as Cohort A, metastatic TNBC). PD-L1 positivity are assessed using the SP142 PD-L1 IHC assay. FIG. 2 illustrates the study design for Cohort A.

Eligible patients (i.e., PD-L1-positive patients) are enrolled to receive tiragolumab (840 mg) and atezolizumab (1680 mg) by IV infusion on Day 1 of every 28-day cycle plus nab-paclitaxel (100 mg/m2) administered to patients by IV infusion on Days 1, 8, and 15 of every 28-day cycle.

In the absence of disease progression or unacceptable toxicity, nab-paclitaxel is administered for a target of at least six cycles, with no maximum.

In order to assess the mechanism of action of the drug combination in the tumor microenvironment and possible resistance mechanisms, tumor tissue may be optionally collected predose on Day 1 of Cycle 2.

To test the mechanisms of resistance to the drug combination in the tumor microenvironment, all patients undergo mandatory tumor biopsy collection (if clinically feasible) at first evidence of disease progression, as assessed by the investigator per RECIST v1.1 (prior to the start of new anti-cancer treatment).

Patients undergo tumor assessments at baseline and every 8 weeks (±7 days) for the first 48 weeks following Day 1 of Cycle 1 regardless of treatment delays. After completion of the Week 48 tumor assessment, tumor assessments are required every 12 weeks (±7 days) regardless of treatment delays until disease progression, as determined by the investigator per RECIST v1.1, withdrawal of consent, death, or study termination, whichever occurs first.

Study treatment is discontinued upon disease progression, as determined by the investigator per RECIST v1.1. For equivocal findings of progression (e.g., very small or uncertain new lesions or lymph nodes; cystic changes or necrosis in existing lesions), treatment may continue until the next scheduled assessment, according to investigator-assessed RECIST v1.1. If at the next scheduled assessment progression is confirmed, the date of progression should be the earlier date when progression was suspected.

B. Dosing and Administration

The treatment regimens are summarized in FIG. 2.

On days of scheduled infusions of atezolizumab, tiragolumab, and chemotherapy, chemotherapy is to be administered after infusion of atezolizumab and tiragolumab.

After the atezolizumab infusion (1680 mg), patients receive 840 mg tiragolumab.

Administration of atezolizumab, tiragolumab, and chemotherapy is performed in a monitored setting where there is immediate access to trained personnel and adequate equipment and medicine to manage potentially serious reactions.

Refer to the pharmacy manual for detailed instructions on drug preparation, storage, and administration.

Tiragolumab and Atezolizumab

Patients in Cohort A receive atezolizumab at a fixed dose of 1680 mg administered by IV infusion Q4W on Day 1 of each 28-day cycle, followed by tiragolumab at a fixed dose of 840 mg administered by IV infusion Q4W on Day 1 of each 28-day cycle. The tiragolumab and atezolizumab doses are fixed and are not dependent on body weight. Tiragolumab and atezolizumab infusions (including observation periods) are administered according to the instructions outlined in Table 24.

TABLE 24
Administration of First and Subsequent Tiragolumab Infusions and Atezolizumab Infusions

| | First Infusion | Subsequent Infusions |
|---|---|---|
| Atezolizumab | No premedication is permitted prior to | If the patient experienced an infusion- |
| infusion | the atezolizumab infusion, | related reaction with any previous |
| | Vital signs (pulse rate, respiratory rate, | infusion, premedication with an |
| | blood pressure, and temperature) are | antihistamine and/or antipyretic |
| | recorded within 60 minutes prior to the | medication may be administered for |
| | infusion, | subsequent doses at the discretion of the |
| | Atezolizumab is infused over 60 (±15) | investigator. |
| | minutes. | Vital signs are recorded within 60 |
| | If clinically indicated, vital signs | minutes prior to the infusion. |
| | should be recorded every 15 (±5) | Atezolizumab is infused over 30 (±10) |
| | minutes during the infusion and at 30 | minutes if the previous infusion was |
| | (±10) minutes after the infusion. | tolerated without an infusion-related |
| | Patients are informed about the | reaction, or 60 (±15) minutes if the |
| | possibility of delayed post-infusion | patient experienced an infusion-related |
| | symptoms and are instructed to contact | reaction with the previous infusion. |
| | their study physician if they develop | If the patient experienced an infusion- |
| | such symptoms. | related reaction with the previous infusion |
| | | or if clinically indicated, vital signs should |
| | | be measured during the infusion and 30 |
| | | (±10) minutes after the infusion. |
| Observation | After the infusion of atezolizumab, the | If the patient tolerated the previous |
| period after | patient begins a 60-minute observation | atezolizumab infusion well without |
| infusion of | period before the tiragolumab infusion, | infusion-associated adverse events, the |
| atezolizumab | Vital signs are recorded at 30 (±10) | observation period before the tiragolumab |
| | minutes after the infusion of | infusion may be reduced to 30 minutes. |
| | atezolizumab. | If the patient experienced infusion- |
| | | associated adverse events in the previous |
| | | infusion, the observation period should be |
| | | 60 minutes. |
| | | If clinically indicated, vital signs should |
| | | be recorded at 15 (±10) minutes after the |
| | | infusion of atezolizumab. |
| Infusion of | No premedication is permitted prior to | If the patient experienced an infusion- |
| tiragolumab | the tiragolumab infusion, | related reaction during any previous |
| | Vital signs (pulse rate, respiratory rate, | infusion of tiragolumab, premedication |
| | blood pressure, and temperature) are | with an antihistamine and/or antipyretic |
| | recorded within 60 minutes prior to the | medication may be administered for |
| | infusion, | subsequent doses, at the discretion of the |
| | Tiragolumab is infused over 60 (±10) | investigator. |
| | minutes. | Vital signs should be recorded within 60 |
| | Vital signs are recorded every 15 (±5) | minutes prior to the tiragolumab infusion. |
| | minutes during the infusion and at 30 | Tiragolumab should be infused over 30 |
| | (±10) minutes after the infusion. | (±10) minutes if the previous infusion was |
| | | tolerated without an infusion-related |
| | | reaction, or 60 (±10) minutes if the |
| | | patient experienced an infusion-related |
| | | reaction with the previous infusion. |
| | | Vital signs should be recorded during |
| | | the infusion if clinically indicated. |
| Observation | After the infusion of tiragolumab, the | If the patient tolerated the previous |
| period after | patient begins a 60-minute observation | infusion of tiragolumab well without |
| infusion of | period before subsequent | infusion-associated adverse events, the |
| tiragolumab | chemotherapy infusions, | observation period before the subsequent |
| | Vital signs should be recorded at 30 | chemotherapy infusions may be reduced |
| | (±10) minutes after the infusion of | to 30 minutes. |
| | tiragolumab. | If clinically indicated, vital signs should |
| | Patients are informed about the | be recorded at 15 (±10) minutes after the |
| | possibility of delayed postinfusion | infusion of tiragolumab. |
| | symptoms and are instructed to contact | Patients are informed about the |
| | their study physician if they develop | possibility of delayed postinfusion |
| | such symptoms. | symptoms and are instructed to contact |
| | | their study physician if they develop such |
| | | symptoms. |

Nab-Paclitaxel

Nab-paclitaxel is administered according to the local prescribing information. The starting dose level of nab-paclitaxel in this study is 100 mg/m² administered to patients intravenously over 30 minutes on Days 1, 8, and 15 of each 28-day cycle (3-weeks on/1-week off schedule). Nab-paclitaxel should be administered after atezolizumab and tiragolumab. Nab-paclitaxel is administered alone on Day 8 and Day 15 of every cycle. Doses of nab-paclitaxel should not be administered more frequently than every 7 days.

Sites should follow their institutional standard of care for determining the nab-paclitaxel dose for patients who are obese and for dose adjustments in the event of patient weight changes. The infusion site should be closely monitored for possible infiltration during drug administration.

In the absence of disease progression or unacceptable toxicity, nab-paclitaxel is administered for a target of at least six cycles, with no maximum.

Refer to the local prescribing information for more details regarding the preparation and administration of nab-paclitaxel.

Dose Modifications

There are no dose modifications for atezolizumab or tiragolumab in this study. For management of nab-paclitaxel (i.e., dose modification and treatment interruption rules), refer to Table 25.

TABLE 25
Suggested Dose Reductions for Nab-Paclitaxel

| | Nab-Paclitaxel |
|---|---|
| Starting dose | 125 mg/m² |
| First dose reduction | 100 mg/m² |
| Second dose reduction | Discontinue |

C. Concomitant Therapy

Concomitant therapy consists of any medication (e.g., prescription drugs, over-the-counter drugs, vaccines, herbal or homeopathic remedies, nutritional supplements) used by a patient in addition to protocol-mandated treatment from 7 days prior to initiation of study drug to the treatment discontinuation visit.

Permitted Therapy

Patients are permitted to use the following therapies during the study:
Oral contraceptives with a failure rate of <1% per year
Hormone-replacement therapy
Prophylactic or therapeutic anticoagulation therapy (such as warfarin at a stable dose or low-molecular-weight heparin)
Inactivated influenza vaccinations
Megestrol acetate administered as an appetite stimulant
Mineralocorticoids (e.g., fludrocortisone)
Corticosteroids administered for chronic obstructive pulmonary disease or asthma
Low-dose corticosteroids administered for orthostatic hypotension or adrenocortical insufficiency
Bisphosphonates for the prevention of skeletal events
Palliative radiotherapy (e.g., treatment of known bony metastases or symptomatic relief of pain) as outlined below:
   Palliative radiotherapy is permitted, provided it does not interfere with the assessment of tumor target lesions (e.g., the lesion to be irradiated must not be the only site of measurable disease). Treatment with tiragolumab and atezolizumab may be continued during palliative radiotherapy. Treatment with nab-paclitaxel should be interrupted per institutional standard of care.

Premedication with antihistamines, antipyretic medications, and/or analgesics may be administered for the second and subsequent atezolizumab and tiragolumab infusions only, at the discretion of the investigator.

Cautionary Therapy

Systemic corticosteroids and TNF-α inhibitors may attenuate potential beneficial immunologic effects of treatment with atezolizumab. Therefore, in situations in which systemic corticosteroids or TNF-α inhibitors would be routinely administered, alternatives, including antihistamines, should be considered. If the alternatives are not feasible, systemic corticosteroids and TNF-α inhibitors may be administered at the discretion of the investigator.

Systemic corticosteroids are recommended, at the discretion of the investigator, for the treatment of specific adverse events when associated with atezolizumab therapy.

Prohibited Therapy

Use of the following concomitant therapies is prohibited as described below:
Concomitant therapy intended for the treatment of cancer (including, but not limited to, chemotherapy, hormonal therapy, immunotherapy, radiotherapy, and herbal therapy), whether health authority-approved or experimental, for various time periods prior to starting study treatment, depending on the agent, and during study treatment, until disease progression is documented and the patient has discontinued study treatment with the exception of palliative radiotherapy
Investigational therapy within 28 days prior to initiation of study treatment and during study treatment
Live, attenuated vaccines (e.g., FLUMIST®) within 4 weeks prior to initiation of study treatment, during atezolizumab treatment, and for 5 months after the final dose of study treatment
Systemic immunostimulatory agents (including, but not limited to, interferons and IL-2) within 4 weeks or 5 drug-elimination half-lives (whichever is longer) prior to initiation of study treatment and during study treatment because these agents could potentially increase the risk for autoimmune conditions when given in combination with atezolizumab and tiragolumab

Systemic immunosuppressive medications (including, but not limited to, azathioprine, methotrexate, and thalidomide) during study treatment because these agents could potentially alter the efficacy and safety of atezolizumab and tiragolumab
Plinabulin, a new molecule being investigated for the reduction of chemotherapy-induced neutropenia, is not allowed as a substitute to G-CSF/GMCSF.

D. Inclusion Criteria

Patients must meet the following general criteria for study entry:
Signed Informed Consent Form
Women and men age 18 years at time of signing Informed Consent Form
Ability to comply with the study protocol
ECOG Performance Status of 0 or 1
Adequate hematologic and end-organ function, defined as the following laboratory results obtained within 14 days prior to initiation of study treatment (Day 1 of Cycle 1):
ANC ≥1.5×109/L (1500/μL) without G-CSF support within 2 weeks prior to Day 1 of Cycle 1
Lymphocyte count ≥0.5×109/L (≥500/μL)
Platelet count ≥100×109/L (≥100,000/μL) without transfusion within 2 weeks prior to Day 1 of Cycle 1
Hemoglobin ≥90 g/L (≥9 g/dL)
   Patients may be transfused or receive erythropoietic treatment to meet this criterion.
AST, ALT, and ALP ≤2.5× upper limit of normal (ULN)
Serum bilirubin ≤1.5×ULN with the following exception:
   Patients with known Gilbert disease who have serum bilirubin level ≤3×ULN may be enrolled.
INR and aPTT≤1.5×ULN
   This applies only to patients who are not receiving therapeutic anticoagulation; patients receiving therapeutic anticoagulation should be on a stable dose.
Creatinine clearance ≥30 mL/min (calculated using the Cockcroft-Gault formula)
Serum albumin ≥25 g/L (≥2.5 g/dL)
Negative HIV test at screening
Negative hepatitis B surface antigen (HBsAg) test at screening
Positive hepatitis B surface antibody (HBsAb) test at screening, or negative HBsAb at screening accompanied by either of the following:
Negative total hepatitis B core antibody (HBcAb)
Positive total HBcAb test followed by quantitative hepatitis B virus (HBV) DNA <500 IU/mL
   The HBV DNA test is performed only for patients who have a negative HBsAg test, a negative HBsAb test, and a positive total HBcAb test.
Negative hepatitis C virus (HCV) antibody test at screening, or positive HCV antibody test followed by a negative HCV RNA test at screening
   The HCV RNA test is performed only for patients who have a positive HCV antibody test.
For women of childbearing potential: agreement to remain abstinent (refrain from heterosexual intercourse) or use contraceptive methods, and agreement to refrain from donating eggs, as defined below:
   Women must remain abstinent or use contraceptive methods that result in a failure rate of <1% per year during the treatment period and for at least 5 months after the final dose of atezolizumab, 90 days after the final dose of tiragolumab, 1 month after the final dose of nab-paclitaxel, 6 months after the final dose of carboplatin or doxorubicin, or 12 months after the final dose of cyclophosphamide, whichever is later. Women must refrain from donating eggs during this same period.
   A woman is considered to be of childbearing potential if she is postmenarcheal, has not reached a postmenopausal state (; 12 continuous months of amenorrhea with no identified cause other than menopause), and has not undergone surgical sterilization (removal of ovaries and/or uterus). Examples of contraceptive methods with a failure rate of <1% per year, when used consistently and correctly, include combined (estrogen and progestogen containing) hormonal contraception associated with inhibition of ovulation, progestogen-only hormonal contraception associated with inhibition of ovulation, bilateral tubal occlusion; male sterilization; intrauterine devices; intrauterine hormone-releasing system; and sexual abstinence.
   The reliability of sexual abstinence should be evaluated in relation to the duration of the clinical study and the preferred and usual lifestyle of the patient.
   Periodic abstinence (e.g., calendar, ovulation, symptothermal, or postovulation methods) and withdrawal are not acceptable methods of contraception.
   Women who would like to become pregnant after discontinuation of study treatment should seek advice about oocyte preservation prior to initiation of study treatment because of the possibility of irreversible infertility due to treatment with chemotherapy.
For men: agreement to remain abstinent (refrain from heterosexual intercourse) or use contraceptive measures and agreement to refrain from donating sperm, as defined below:
   With female partners of childbearing potential or pregnant female partners, men must remain abstinent or use a condom plus an additional contraceptive method that together result in a failure rate of <1% per year during the treatment period and for 90 days after the final dose of tiragolumab, or 6 months after the final dose of nab-paclitaxel, carboplatin, doxorubicin, or cyclophosphamide, whichever is later. Men must refrain from donating sperm during this same period.
   The reliability of sexual abstinence should be evaluated in relation to the duration of the clinical trial and the preferred and usual lifestyle of the patient.
   Periodic abstinence (e.g., calendar, ovulation, symptothermal, or postovulation methods) and withdrawal are not acceptable methods of contraception.
   Men who would like to father a child after initiation of study treatment should seek advice about sperm preservation prior to initiation of study treatment because of the possibility of irreversible infertility due to treatment with chemotherapy.
For women who are not postmenopausal (≥12 months of non-therapy-induced amenorrhea) or have undergone a sterilization procedure, a negative serum pregnancy test result is required within 14 days prior to initiation of study treatment
Willingness and ability to comply with scheduled visits, treatment plans, laboratory tests, and other study procedures

Patients in Cohort A must meet the following cancer-specific criteria for study entry:
Life expectancy ≥12 weeks
Metastatic or locally advanced unresectable, histologically documented TNBC (absence of HER2, ER, and progesterone receptor (PgR) expression, as determined by local testing)

HER2 negativity is defined as either of the following (Wolff et al. 2018) by local laboratory assessment:
In situ hybridization (ISH) non-amplified (ratio of HER2 to CEP17<2.0 or single probe average HER2 gene copy number <4 signals/cell), or IHC 0 or IHC 1+.
ER and PgR negativity is defined as <1% of cells expressing hormonal receptors, as determined by IHC analysis.
Only patients with metastatic TNBC tumors that are centrally tested and found to be PD-L1 positive are enrolled

A tumor specimen obtained from relapsed metastatic or locally advanced disease (if applicable) must be submitted, if clinically feasible.

Representative FFPE tumor specimen (either an archival specimen or fresh pretreatment tissue from relapsed disease) in paraffin blocks (preferred) or at least 20 unstained slides

Tumor tissue should be of good quality based on total and viable tumor content and must be evaluated for PD-L1 expression, as determined using Ventana (SP142) PD-L1 IHC assay prior to enrollment, with positivity defined as ≥1% of the tumor area occupied by PD-L1-expressing tumor-infiltrating immune cells of any intensity as determined by the central laboratory. Patients whose tumor tissue is not evaluable for PD-L1 expression are not eligible.

If multiple tumor specimens are submitted, patients may be eligible if at least one specimen is evaluable and positive for PD-L1 expression (regardless whether the tissue is from an archival specimen or from relapsed disease).

Acceptable samples include core-needle biopsies for deep tumor tissue (a minimum of three cores) or excisional, incisional, punch, or forceps biopsies for cutaneous, subcutaneous, or mucosal lesions.

FFPE tumor specimens in paraffin blocks are preferred.

Fine-needle aspiration, brushing, cell pellet from pleural effusion, bone metastases, and lavage samples are not acceptable. Tumor tissue from bone metastases is not evaluable for PD-L1 expression and is therefore not acceptable.
No prior chemotherapy or targeted systemic therapy for inoperable, locally advanced, or metastatic TNBC

Prior radiotherapy for metastatic disease is permitted. There is no required minimum washout period for radiotherapy. Patients should be recovered from the effects of radiation.

Prior chemotherapy (including taxanes) and/or CIT (anti-PD-L1 or anti-PD-1 agents only) in the neoadjuvant or adjuvant setting are allowable if treatment was completed ≥12 months prior to initiation of study treatment.
Measurable disease, as assessed by the investigator according to RECIST v1.1

Previously irradiated lesions can be considered as measurable disease only if disease progression has been unequivocally documented at that site since radiation.

E. Exclusion Criteria

Patients who meet any of the following criteria are excluded from study:
Pregnancy or breastfeeding, or intention of becoming pregnant during study treatment, within 90 days after tiragolumab treatment, within 5 months after atezolizumab treatment, within 6 months after nab-paclitaxel, carboplatin, or doxorubicin treatment, or within 12 months after cyclophosphamide treatment, whichever is longer
Women of childbearing potential must have a negative serum pregnancy test result within 14 days prior to initiation of study treatment.
Evidence of significant uncontrolled concurrent disease that could affect compliance with the protocol or interpretation of results, including significant liver disease (such as cirrhosis, uncontrolled major seizure disorder, or superior vena cava syndrome)
Significant cardiovascular disease, such as New York Heart Association cardiac disease (Class II or greater), myocardial infarction within 3 months prior to initiation of study treatment, unstable arrhythmias, or unstable angina
Patients with a known LVEF <53% are excluded.
Patients with known coronary artery disease or congestive heart failure who do not meet the criteria above.
Severe infection within 4 weeks prior to initiation of study treatment, including, but not limited to, hospitalization for complications of infection, bacteremia, or severe pneumonia
Treatment with oral or IV antibiotics within 2 weeks prior to initiation of study treatment Patients receiving routine antibiotic prophylaxis (e.g., to prevent chronic obstructive pulmonary disease exacerbation or for dental extraction) are eligible.
Major surgical procedure within 28 days prior to initiation of study treatment or anticipation of the need for a major surgical procedure during the study other than for diagnosis
   Placement of central venous access catheter(s) (e.g., port or similar) is not considered a major surgical procedure and is therefore permitted.
History of severe allergic, anaphylactic, or other hypersensitivity reactions to chimeric or humanized antibodies or fusion proteins
Known hypersensitivity or allergy to biopharmaceuticals produced in CHO cells or any component of the tiragolumab or atezolizumab formulation
Active or history of autoimmune disease, including, but not limited to, myasthenia gravis, myositis, autoimmune hepatitis, systemic lupus erythematosus, rheumatoid arthritis, inflammatory bowel disease, vascular thrombosis associated with antiphospholipid syndrome, Wegener's granulomatosis, Sjögren syndrome, Guillain-Barré syndrome, multiple sclerosis, vasculitis, or glomerulonephritis
   Patients with a history of autoimmune-mediated hypothyroidism on a stable dose of thyroid replacement hormone are eligible for this study.
Patients with controlled Type 1 diabetes mellitus on a stable insulin dosing regimen are eligible for this study.
Patients with eczema, psoriasis, lichen simplex chronicus or vitiligo with dermatologic manifestations only (e.g., no psoriatic arthritis) are permitted provided that they meet the following conditions:
   Rash must cover <10% of body surface area
   Disease is well controlled at baseline and requires only low-potency topical corticosteroids
   No occurrence of acute exacerbations of the underlying condition requiring psoralen plus ultraviolet A radiation, methotrexate, retinoids, biologic agents, oral calcineurin inhibitors, or high-potency or oral corticosteroids within the previous 12 months
   Caution should be used when considering atezolizumab for patients who have previously experienced a severe or life-threatening skin adverse reaction while receiving another immunostimulatory anti-cancer agent.
Prior allogeneic stem cell or solid organ transplantation
History of idiopathic pulmonary fibrosis (including pneumonitis), drug-induced pneumonitis, organizing pneumonia (i.e., bronchiolitis obliterans, cryptogenic organizing pneumonia), or evidence of active pneumonitis on the chest computed tomography (CT) scan at screening
   History of radiation pneumonitis in the radiation field (fibrosis) is permitted.
Positive EBV viral capsid antigen (VCA) IgM test at screening
   An EBV polymerase chain reaction (PCR) test should be performed as clinically indicated to screen for active infection or suspected chronic active infection.
   Patients with a positive EBV PCR test are excluded.
Active tuberculosis (TB)
Receipt of a live, attenuated vaccine within 4 weeks prior to initiation of study treatment or anticipation that such a live, attenuated vaccine is required during the study
   Patients must agree not to receive live, attenuated vaccine (e.g., FLUMIST®) within 28 days prior to initiation of study treatment, during treatment, or within 5 months following the final dose of study treatment.
Prior treatment with CD137 agonists or immune checkpoint blockade therapies, including anti-CTLA-4, with an exception of anti-PD-1, or anti-PD-L1 therapeutic antibodies
Treatment with systemic immunostimulatory agents (including, but not limited to, interferons or IL-2) within 4 weeks or 5 drug-elimination half-lives of the drug (whichever is longer) prior to initiation of study treatment
Treatment with systemic immunosuppressive medications (including, but not limited to, prednisone, dexamethasone, azathioprine, methotrexate, thalidomide, and anti-tumor necrosis factor [anti-TNF] agents) within 2 weeks prior to initiation of study treatment, or anticipated requirement for systemic immunosuppressive medications during the study
   Patients who have received acute, low-dose, systemic immunosuppressant medications (e.g., a one-time dose of dexamethasone for nausea) may be enrolled in the study.
   Patients with a history of allergic reaction to IV contrast requiring steroid pretreatment should have baseline and subsequent tumor assessments performed using magnetic resonance imaging (MRI)
   The use of inhaled corticosteroids for chronic obstructive pulmonary disease, mineralocorticoids (e.g., fludrocortisone) for patients with orthostatic hypotension, and low-dose supplemental corticosteroids for adrenocortical insufficiency is allowed.

Patients in Cohort A who meet any of the following cancer-specific exclusion criteria are excluded from study entry:
FFPE tumor tissue that is PD-L1 negative, as determined on the SP142 PD-L1 IHC assay, with positivity defined as ≥1% of the tumor area occupied by PD-L1-expressing tumor-infiltrating immune cells of any intensity
Spinal cord compression not definitively treated with surgery and/or radiation or previously diagnosed and treated spinal cord compression without evidence that disease has been clinically stable for >2 weeks prior to initiation of study treatment
Known central nervous system (CNS) disease, except for treated asymptomatic CNS metastases, provided all of the following criteria are met:
   Only supratentorial and cerebellar metastases allowed (i.e., no metastases to midbrain, pons, medulla, or spinal cord)
   No ongoing requirement for corticosteroids as therapy for CNS disease
   No stereotactic radiation within 7 days or whole brain radiation within 14 days prior to initiation of study treatment
   No evidence of interim progression between the completion of CNS-directed therapy and the screening radiographic study
   Note: Patients with new asymptomatic CNS metastases detected on the screening scan must receive radiotherapy and/or surgery for CNS metastases. Following treatment, these patients may be eligible without the need for an additional brain scan prior to enrollment, if all of the other criteria are met.
Leptomeningeal disease
Uncontrolled pleural effusion, pericardial effusion, or ascites Patients with indwelling catheters (e.g., PLEURX®) are allowed.
Uncontrolled tumor-related pain

Patients requiring narcotic pain medication must be on a stable regimen at study entry.

Symptomatic lesions (e.g., bone metastases or metastases causing nerve impingement) amenable to palliative radiotherapy should be treated prior to enrollment. Patients should be recovered from the effects of radiation. There is no required minimum recovery period.

Asymptomatic metastatic lesions whose further growth would likely cause functional deficits or intractable pain (e.g., epidural metastasis that is not presently associated with spinal cord compression) should be considered for locoregional therapy if appropriate prior to enrollment.
Uncontrolled hypercalcemia (>1.5 mmol/L ionized calcium or calcium >12 mg/dL or corrected serum calcium greater than ULN) or symptomatic hypercalcemia requiring continued use of bisphosphonate therapy

Patients who are receiving bisphosphonate therapy specifically to prevent skeletal events and who do not have a history of clinically significant hypercalcemia are eligible.
Malignancies other than TNBC within 5 years prior to initiation of study treatment, with the exception of those with a negligible risk of metastasis or death (e.g., 5-year OS of >90%) and treated with expected curative outcome (such as adequately treated carcinoma in situ of the cervix or basal, localized prostate cancer or squamous cell skin cancer)
Known hypersensitivity to nab-paclitaxel or to any of its excipients

F. Analysis

Safety analyses are conducted in all patients.

Efficacy analyses use an ITT approach for Cohorts A and B, wherein any enrolled patient is included in the analysis regardless of whether the patient receives any assigned study drug. Analyses based on subsets of the ITT population might also be conducted.

Hypothesis tests are two sided unless otherwise indicated.

Sample Size

The objective of Cohort A is to estimate the effect of tiragolumab combined with atezolizumab and nab-paclitaxel on the primary efficacy endpoint, defined as objective response. Compared with historical data, the proportion of response is considered promising or not promising for further development. There is no formal hypothesis testing. Statistics are presented with a two-sided 90% CI. When making a recommendation to continue or stop the development, the totality of the data is considered, including the results on secondary efficacy endpoints, including, but not restricted, to PFS, OS, and results of analyses on subgroups.

The primary efficacy analysis is the estimation of the true proportion of patients expected to obtain a CR or a PR (i.e., ORR). Data from completed and ongoing studies in similar disease settings could be used as historical controls for comparison. Currently available data indicate that the historical confirmed response rate is approximately 53% (TECENTRIQ® U.S. Package Insert) in locally advanced unresectable or metastatic PD-L1-positive TNBC.

A sample size of 40 patients is deemed sufficient to provide adequate precision for the point estimate and for the lower bound of the two-sided 90% CI to rule out a clinically uninteresting probability of response of <53%, assuming an observed response rate of 67.5%. Specifically, if at least 27 of the 40 patients achieve a response, it can be concluded that the response rate is higher than 53%. Updated estimates of the proportion of patients expected to achieve a response are expected to be available from ongoing studies by the time of the efficacy analysis and are used as reference data.

Table 26 lists the two-sided 90% Clopper-Pearson exact CI for the true probability of achieving a response for a range of observed proportions based on a sample of 40 patients. The probabilities of detecting positive and negative signals based on observed point estimate of ORR under various thresholds as well as underlying true ORRs are given in Table 27 and Table 28.

TABLE 26
Two -Sided Clopper-Pearson Exact Confidence Interval for the
True Probability of Achieving a Response

| | Two-Sided 90% Clopper-Pearson |
|---|---|
| Observed Proportion of Patients | Confidence Interval for a True |
| Achieving a Response (n = 40) | Population Response (n = 40) |
| p_{Estimated} = 0.675 (27 of 40 patients) | 0.53370 ≤ p_{Estimated} ≤ 0.79587 |
| p_{Estimated} = 0.700 (28 of 40 patients) | 0.55972 ≤ p_{Estimated} ≤ 0.81688 |
| p_{Estimated} = 0.725 (29 of 40 patients) | 0.58612 ≤ p_{Estimated} ≤ 0.83748 |
| p_{Estimated} = 0.750 (30 of 40 patients) | 0.61294 ≤ p_{Estimated} ≤ 0.85763 |

TABLE 27
Probability of Declaring Positive Signal (Observed
ORR Greater Than or Equal to the Chosen Threshold)

| | Probability of Observed |
|---|---|
| True Underlying ORR | Response Proportion > 73% |
| 53% | 0.4% |
| 68% | 22.1% |
| 73% | 46.8% |

Note:
Results are based on simulation with 1e6 runs.

TABLE 28
Probability of Declaring Negative Signal
(Observed ORR Less Than the Chosen Threshold)

| | Probability | Probability |
|---|---|---|
| True | of Observed | of Observed |
| Underlying | Response | Response |
| ORR | Proportion < 58% | Proportion < 63% |
| 53% | 76.6% | 91.4% |
| 68% | 10.7% | 27.8% |
| 73% | 2.5% | 9.6% |

Safety Analysis

Safety assessments include the incidence, nature, and severity of adverse events, protocol-mandated vital signs, laboratory abnormalities, and other protocol-specified tests that are deemed critical to the safety evaluation of the study. Adverse events are graded according to the NCI CTCAE v5.0.

Safety is assessed through summaries of adverse events, changes in laboratory test results, changes in vital signs, study treatment exposures, and immunogenicity as measured by ADAs and is presented by treatment arm.

Verbatim descriptions of adverse events are mapped to MedDRA terms.

Treatment-emergent events (defined as events occurring on or after the first dose of study treatment are summarized by MedDRA term, appropriate MedDRA levels, and NCI CTCAE v5.0 grade, regardless of relationship to study drug as assessed by the investigator. For each patient, if multiple incidences of the same adverse events occur, the maximum severity reported is used in the summaries.

The following treatment-emergent adverse events are summarized separately: adverse events leading to withdrawal of study drug, adverse events leading to dose reduction or interruption, Grade ≥3 adverse events, Grade 5 adverse events, serious adverse events, and adverse events of special interest.

All deaths and causes of death are summarized.

Relevant laboratory values are summarized by timepoint, with NCI CTCAE Grade 3 and Grade 4 values identified, where appropriate. Changes in NCI CTCAE grade are tabulated by treatment arm.

Analyses of Exposure, Adverse Events, Laboratory, and Vital Sign Data

Safety is assessed through summaries of exposure to study treatment, adverse events, changes in laboratory test results, and changes in vital signs and ECGs.

Study treatment exposure (such as treatment duration, total dose received, and number of cycles and dose modifications) is summarized with descriptive statistics.

All verbatim adverse event terms are mapped to MedDRA thesaurus terms, and adverse event severity is graded according to NCI CTCAE v5.0. All adverse events, serious adverse events, adverse events leading to death, adverse events of special interest, and adverse events leading to study treatment discontinuation that occur on or after the first dose of study treatment (i.e., treatment-emergent adverse events) are summarized by mapped term, appropriate thesaurus level, and severity grade. For events of varying severity, the highest grade is used in the summaries. Deaths and cause of death are summarized.

Relevant laboratory, vital sign (pulse rate, respiratory rate, blood pressure, pulse oximetry, and temperature), and ECG data are displayed by time, with grades identified where appropriate. Additionally, a shift table of selected laboratory tests is used to summarize the baseline and maximum postbaseline severity grade. Changes in vital signs and ECGs are summarized.

Efficacy Analysis

Primary Efficacy Objective

The primary efficacy objective for Cohort A is to assess the efficacy of tiragolumab combined with atezolizumab and nab-paclitaxel on the basis of the following endpoint:
ORR, defined as the proportion of patients with a CR or PR, as determined by the investigator using RECIST v1.1
   Confirmation of a PR or CR is required. Patients not meeting this criterion, including patients without any postbaseline tumor assessment, are considered as non-responders. ORR is defined as the proportion of patients who have an objective response.
   The earliest look at efficacy data occurs once all patients have undergone at least two tumor assessments.

Secondary Efficacy Objective

The secondary efficacy objective for Cohort A is to obtain preliminary data on the efficacy of tiragolumab combined with atezolizumab and nab-paclitaxel on the basis of the following endpoints:
PFS, defined as the time from enrollment to the first occurrence of disease progression or death from any cause (whichever occurs first), as determined by the investigator according to RECIST v1.1
   Data for patients who have not experienced disease progression or died are censored on the date on which they are last known to be alive and progression-free prior to or on the clinical cutoff date for the respective analysis. Patients with no postbaseline information are censored at the date of enrollment.
DOR, defined as the time from the first occurrence of a documented objective response to disease progression or death from any cause (whichever occurs first), as determined by the investigator according to RECIST v1.1
   Data for patients who have not experienced disease progression or died are censored at the last tumor assessment date. If no tumor assessments are performed after the date of the first occurrence of a CR or PR, data for DOR are censored at the date of the first occurrence of a CR or PR.
OS, defined as the time from enrollment to death from any cause
   Patients who are not reported as having died at the time of analyses are censored at the date when they were last known to be alive. Patients who do not have information after baseline are censored at the date of enrollment.

Pharmacokinetic Analysis

Samples are collected for PK analyses and to compare exposure in this study with that attained in previous studies. Serum concentrations of tiragolumab and atezolizumab and plasma concentrations of nab-paclitaxel, carboplatin, doxorubicin, and cyclophosphamide are reported as individual values and summarized (mean, standard deviation, coefficient of variation, median, range, geometric mean, and geometric mean coefficient of variation) by treatment arm and cycle, when appropriate and as data allow.

Individual and median serum tiragolumab and atezolizumab concentrations are plotted by treatment arm and day. Tiragolumab and atezolizumab concentration data may be pooled with data from other studies using an established population-PK model to derive PK parameters such as clearance, volume of distribution, and AUC, as warranted by the data. Potential correlations of relevant PK parameters with dose, safety, efficacy, or biomarker outcomes may be explored.

Immunogenicity Analysis

The immunogenicity analyses include patients with any ADA assessments, with patients grouped according to treatment received. The number and proportion of treatment-emergent ADA-positive patients and ADA-negative patients during both the treatment and follow-up periods are summarized by treatment arm.

The relationship between ADA status and safety, efficacy, and PK endpoints may be analyzed and reported by means of descriptive statistics.

Biomarker Analysis

Although no formal statistical analyses of exploratory biomarkers are performed, data may be analyzed in the context of this study and in aggregate with data from other studies.

### Example 4. A Phase Ia/IB Open-Label, Dose-Escalation Study of the Safety and Pharmacokinetics of Tiragolumab as a Single Agent and in Combination with Atezolizumab and/or Other Anti-Cancer Therapies in Patients with Locally Advanced or Metastatic Tumors

The present example describes a first-in-human Phase I open-label, multicenter, global, dose-escalation study designed to evaluate:

(Phase Ia) the safety, tolerability, and PK of tiragolumab as a single agent in patients with locally advanced, recurrent, or metastatic incurable tumors for whom standard therapy does not exist; has proven to be ineffective or intolerable or is considered inappropriate; or for whom a clinical trial of an investigational agent is a recognized standard of care; and

(Phase Ib) the safety, tolerability, and PK of tiragolumab when administered in combination with standard of care therapy in patients with locally advanced, recurrent, or metastatic incurable tumors for whom standard therapy does not exist, has proven to be ineffective or intolerable, or is considered inappropriate; or in patients for whom a clinical trial of an investigational agent is a recognized standard of care or for whom a clinical trial of an investigational agent in combination with an anti-PD-L1 antibody is considered an acceptable treatment option.

A. Study Design

This first-in-human Phase I open-label, multicenter, global, dose-escalation study is designed to evaluate the safety, tolerability, and PK of tiragolumab as a single agent in patients with locally advanced, recurrent, or metastatic incurable tumors for whom standard therapy does not exist; has proven to be ineffective or intolerable or is considered inappropriate; or for whom a clinical trial of an investigational agent is a recognized standard of care. This is assessed in the Phase Ia portion of the study.

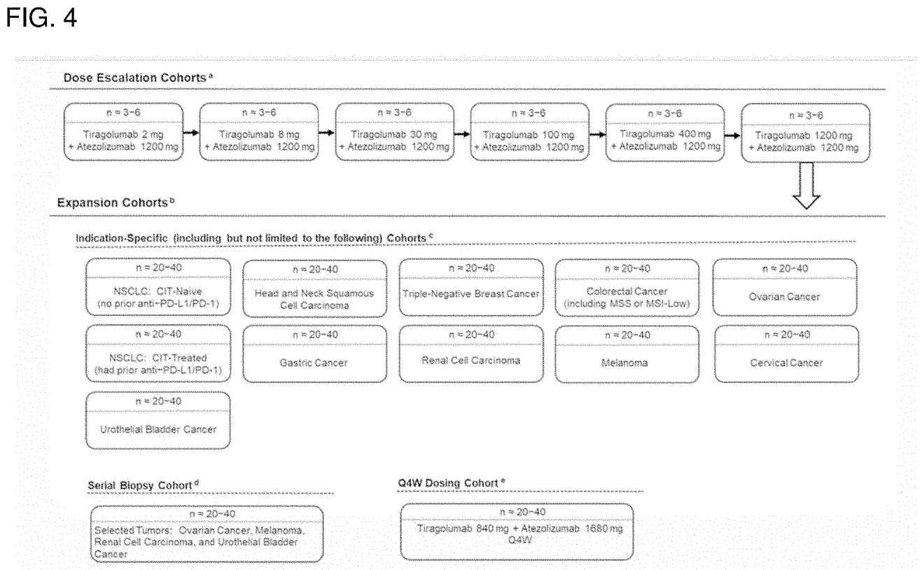

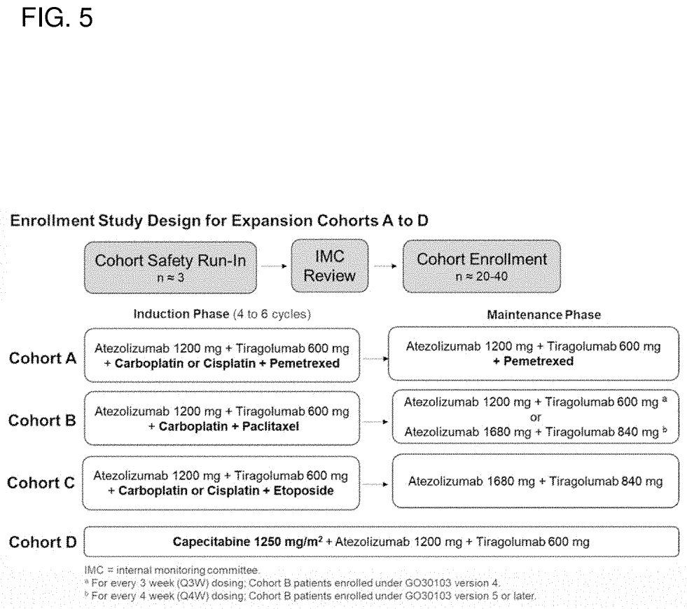

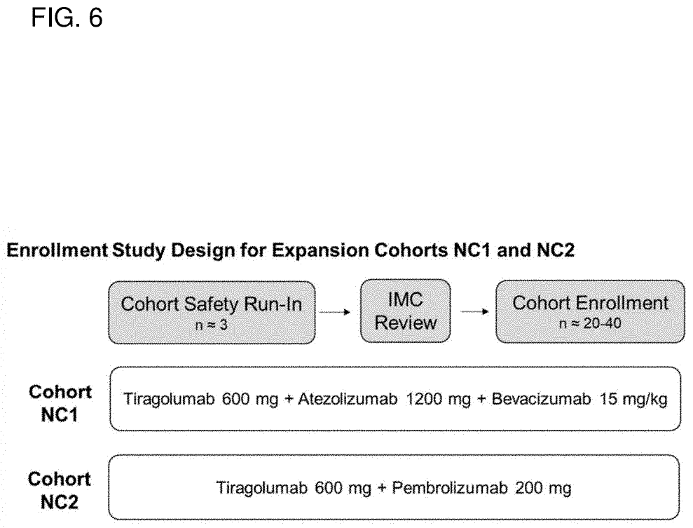

This study is also designed to enable evaluation of the safety, tolerability, and PK of tiragolumab when administered in combination with standard of care therapy in patients with locally advanced, recurrent, or metastatic incurable tumors for whom standard therapy does not exist, has proven to be ineffective or intolerable, or is considered inappropriate; or in patients for whom a clinical trial of an investigational agent is a recognized standard of care or for whom a clinical trial of an investigational agent in combination with an anti-PD-L1 antibody is considered an acceptable treatment option. This is studied in multiple cohorts in the Phase Ib portion of the study:
Phase Ib: tiragolumab and atezolizumab dose-escalation cohorts (FIG. 4)
Phase Ib: tiragolumab and atezolizumab indication-specific expansion cohorts (FIG. 4)
Phase Ib: tiragolumab and atezolizumab serial biopsy cohort (FIG. 4)
Phase Ib: tiragolumab and atezolizumab every 4 week (Q4W) dosing expansion cohort (FIG. 4)
Phase Ib: tiragolumab and atezolizumab combined with chemotherapy expansion cohorts (Cohorts A to D; FIG. 5)
Phase Ib: tiragolumab combined with non-chemotherapy expansion cohorts (Cohorts NC1 and NC2; FIG. 6)

The Phase Ib portion of this study is activated after DLT evaluation of at least two dose levels of single-agent tiragolumab has been completed and all relevant single-agent safety data have been thoroughly reviewed with the investigators as well as by an IMC. If it is deemed appropriate, the Phase Ib portion initiates at a dose level no higher than the initial tiragolumab dose level, using the same dose-escalation scheme and comprehensive safety monitoring plan, as in the Phase Ia portion. In addition, patients who progress on the Phase Ia portion of the study with single-agent tiragolumab may be given the option to crossover into the Phase Ib portion of the study.

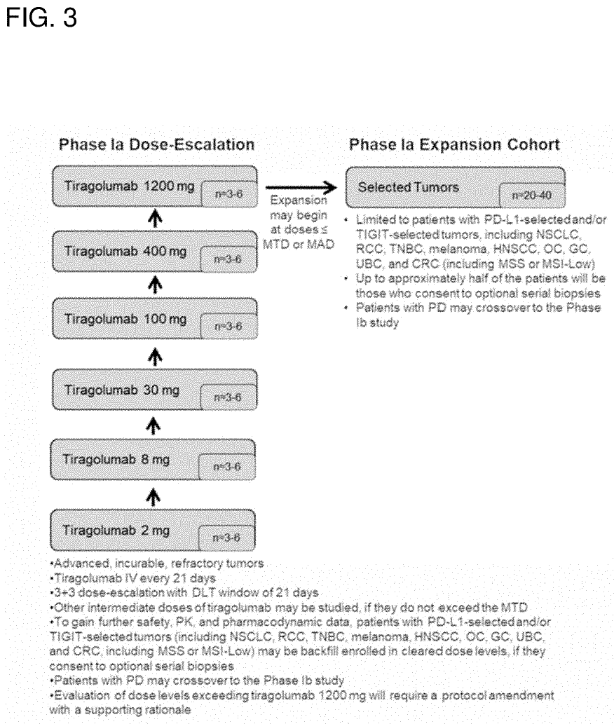

Both the Phase Ia and Phase Ib portions of the study consist of a screening period, a treatment period, and a post-treatment follow-up period. Patients are enrolled in two stages, a dose-escalation stage and a dose-expansion stage; see FIG. 3 and FIG. 4 for Phase Ia and Phase Ib, respectively. FIG. 4 additionally shows the serial biopsy and Q4W cohorts, while FIG. 5 shows the Phase Ib chemotherapy expansion cohorts, and FIG. 6 shows the Phase Ib non-chemotherapy expansion cohorts. During the dose-escalation stage, cohorts of approximately 3-6 patients each are evaluated at escalating dose levels to determine the MTD or maximum administered dose (MAD) for tiragolumab as a single agent or in combination with atezolizumab. To acquire additional safety and pharmacodynamic data to more fully inform the dose selection for the expansion cohorts, additional patients may be enrolled to backfill cohorts at dose levels that have been shown to not exceed the relevant MTD based on the dose-escalation criteria described below. For the purposes of dose-escalation decisions, these patients are not included as part of the DLT-evaluable population.

In the dose-expansion stage, patients are enrolled and treated at or below the MTD or MAD of tiragolumab as a single agent (Phase Ia) or in combination with atezolizumab and/or other anti-cancer therapies (Phase Ib).

Tiragolumab as a single agent (Phase Ia), or the combinations of tiragolumab and atezolizumab, tiragolumab and atezolizumab with bevacizumab, or tiragolumab and pembrolizumab (Phase Ib cohorts without chemotherapy) are administered by IV infusion on Day 1 of each 21-day cycle or on Day 1 of each 28-day cycle (Phase Ib Q4W dosing expansion). Tiragolumab is administered prior to atezolizumab, bevacizumab, or pembrolizumab in the Phase Ib cohorts without chemotherapy. In the absence of unacceptable toxicity or clinically compelling evidence of disease progression, treatment with either tiragolumab (Phase Ia) or tiragolumab in combination with atezolizumab and/or other anti-cancer therapies (Phase Ib expansion cohorts) may be continued beyond Cycle 1 based on a favorable assessment of benefit and risk by the investigator.

In the Phase Ib chemotherapy and non-chemotherapy expansion cohorts and the Phase Ib Q4W dosing expansion cohort, a safety run-in of 3 patients is completed. All relevant safety data from the safety run-in are thoroughly reviewed by an IMC and with the investigators before enrollment is continued.

Tiragolumab and atezolizumab are combined with specific chemotherapy regimens in each of the four chemotherapy expansion cohorts: carboplatin or cisplatin and pemetrexed in Cohort A, carboplatin and paclitaxel in Cohort B, carboplatin or cisplatin and etoposide in Cohort C, and capecitabine in Cohort D. In Cohorts A, B, and C, treatment consists of an induction phase and a maintenance phase. In the induction phase, the combination of tiragolumab and atezolizumab with chemotherapy is administered by IV infusion on a 21-day cycle for 4 to 6 cycles for Cohorts A and B and for 4 cycles for Cohort C. The number of cycles of induction treatment for Cohorts A and B are at the discretion of the investigator.

Following the induction phase, patients who have not experienced disease progression or unacceptable toxicity continue treatment with maintenance therapy. During the maintenance phase, patients in Cohorts B and C continue tiragolumab and atezolizumab only, while patients in Cohort A continue tiragolumab and atezolizumab with pemetrexed.

In the maintenance phase, Cohort A continues on a 21-day cycle with atezolizumab, tiragolumab, and pemetrexed. In Cohort B, patients that enrolled under protocol GO30103 Version 4 continue a 21-day cycle with atezolizumab and tiragolumab. In Cohort B, patients who enrolled under protocol GO301 03 Version 5 or later, start a 28-day cycle with atezolizumab and tiragolumab. Cohort C starts a 28-day cycle with atezolizumab and tiragolumab.

In Cohort D, patients receive treatment with tiragolumab, atezolizumab, and capecitabine until unacceptable toxicity or loss of clinical benefit as determined by the investigator. Patients receive capecitabine at a dose of 1250 mg/m² twice a day (BID) orally on Days 1 through 14 of each 21-day cycle. On Day 1 of Cycle 1, the first dose of capecitabine is administered at the clinic prior to the atezolizumab and tiragolumab infusion. The combination of tiragolumab and atezolizumab is administered by IV infusion on a 21-day cycle.

In all of the chemotherapy expansion cohorts, atezolizumab is administered prior to tiragolumab.

All patients are closely monitored for adverse events throughout the study. Adverse events are graded according to the NCI CTCAE, Version 4.0.

To characterize the PK properties, immunogenic response, and pharmacodynamic effects of tiragolumab as a single agent (Phase Ia) or in combination with atezolizumab with and without chemotherapy (Phase Ib), blood samples are taken at various timepoints before and after dosing. Depending on the results from the interim PK analyses, the frequency of PK sampling may be reduced later in the study.

Patients undergo tumor assessments at screening and during the study, which are measured by standard Response Evaluation Criteria in Solid Tumors (RECIST) v1.1 criteria.

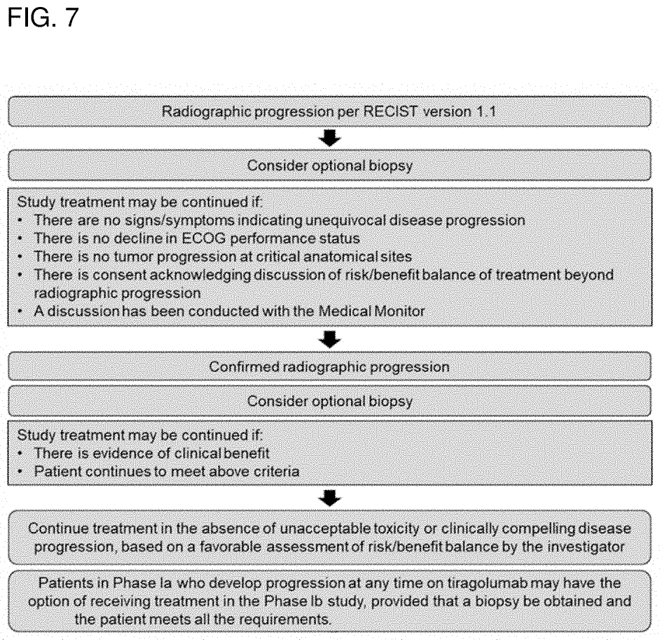

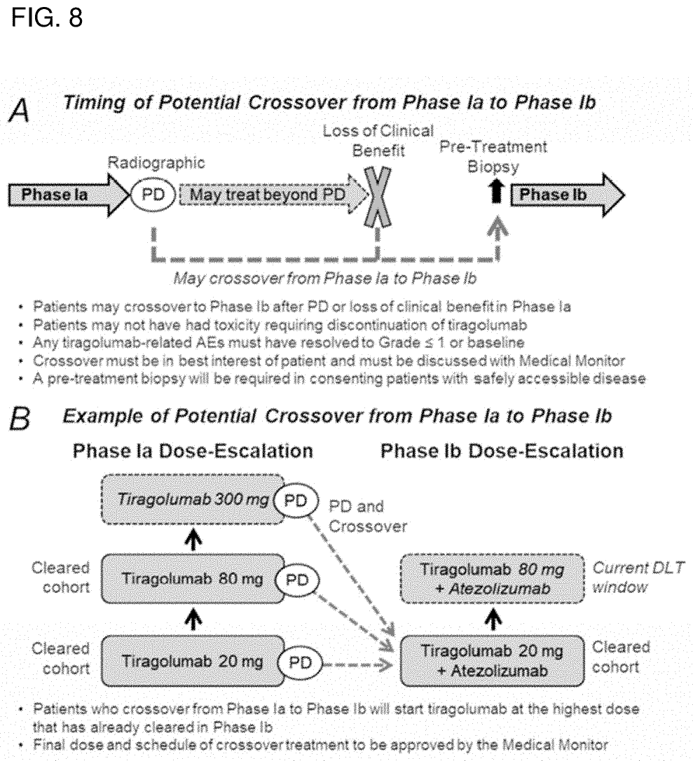

Patients may be permitted to continue study treatment even if standard RECIST v1.1 criteria for progression of disease are met in the Phase Ia or Phase Ib portions of the study, provided that they meet the criteria for continued treatment (FIG. 7). Patients who discontinue the Phase Ia portion of the study may be permitted to cross over into the Phase Ib portion of the study and receive treatment with tiragolumab in combination with atezolizumab, provided that they meet the criteria for crossover and consent to a biopsy of an accessible lesion (FIG. 8).

Patients who permanently discontinue tiragolumab (Phase Ia) or tiragolumab and atezolizumab, tiragolumab and atezolizumab with chemotherapy, tiragolumab and atezolizumab with bevacizumab, or tiragolumab and pembrolizumab (Phase Ib) are to return to the clinic for a treatment discontinuation visit within 30 days after the last dose of study treatment. Further monitoring and recording of adverse events of special interest occurs for up to 90 days after the last dose of study treatment regardless of initiation of another systemic anti-cancer therapy. Monitoring and recording of all other adverse events occurs for up to 90 days after the last dose of study treatment or until initiation of another systemic anti-cancer therapy, whichever occurs first. All patients in the study are followed for survival and subsequent anti-cancer therapy information approximately every 3 months until death, loss to follow-up, or study termination, unless the patient requests to be withdrawn from follow-up.

An IMC periodically evaluates the accumulating safety data from all patients treated in this study. Safety summary data is evaluated by the IMC after DLT assessment has been completed for the first two dose cohorts in the Phase Ia (single-agent tiragolumab) portion of the study. The IMC reviews the safety data to make a recommendation whether to continue enrollment without changes to the protocol in Phase Ia, to begin enrollment in the Phase Ib portion of the study with tiragolumab in combination with atezolizumab to review safety data from safety run-in cohorts and make a recommendation whether to continue enrollment in the Phase Ib chemotherapy expansion cohorts, non-chemotherapy expansion cohorts, and the Q4W dosing expansion cohort, to modify the safety monitoring and/or eligibility criteria, to add additional safety review to address emerging safety issues, or to terminate the study. In the absence of safety concerns that would preclude it, accrual continues in the Phase Ia portion of the study while the safety analysis is ongoing.

To further mitigate the risk of potential unexpected adverse events, the IMC also reviews safety data from patients entered in the expansion cohorts of the Phase Ia and Phase Ib portions of the study on a regular basis, specifically to make recommendations regarding study conduct on the basis of emerging trial safety data to ensure patient safety while receiving study treatment. Interim analyses for futility by the IMC also occur in the expansion cohorts in Phase Ia and Phase Ib. In addition, the Medical Monitor may request additional safety reports and may call for ad-hoc meetings of the IMC at any time during the study to review ongoing safety data for a risk-benefit balance.

During the dose-escalation stage, at least 3 patients are treated at each tiragolumab dose level in accordance with the dose-escalation rules described below to determine the MTD or MAD of tiragolumab as a single agent or in combination with atezolizumab. Enrollment of the first 3 patients in each new dose-escalation cohort examining a new dose level of tiragolumab in Phase Ia or Phase Ib is staggered such that their respective Cycle 1, Day 1 treatments are administered ≥7 days apart. All patients are closely monitored for adverse events during a DLT assessment window, defined as 21 days (Days 1-21 of Cycle 1).

Any dose-escalation patient who does not complete the DLT assessment window for a reason other than a DLT is considered non-evaluable for dose-escalation decisions as well as for the MTD assessment and may be replaced by an additional patient at that same dose level. Patients who receive supportive care during the DLT assessment window that confounds the evaluation of DLTs (not including supportive care described below) and do not experience a DLT may be replaced at the discretion of the Medical Monitor. A patient who has any component of study treatment held during the DLT assessment window for a reason other than a DLT such that administration of the next planned dose is delayed by more than 7 days, is considered non-evaluable for dose-escalation decisions and for the MTD or MAD assessment and may be replaced by an additional patient at that same dose level.

Patients who are treated on the Phase Ia portion of the study and who develop disease progression on tiragolumab alone may be eligible to receive treatment on the Phase Ib portion of the study (FIG. 8), provided that specific eligibility criteria for crossover are met.

Definition of Dose-Limiting Toxicity

In both the Phase Ia and Phase Ib portions of the study, any one of the following events are considered a DLT if it occurs during the DLT assessment window and is assessed by the investigator to be related to study treatment:
Grade ≥3 non-hematologic, non-hepatic adverse event, with the following exceptions:
   Grade 3 nausea, vomiting, or diarrhea that resolves to Grade ≤2 with standard-of-care therapy in ≤3 days
   Grade 3 fatigue that resolves to Grade ≤2 in ≤3 days
   Grade 3 fever (as defined by >40° C. for ≤24 hours)
   Grade 3 adverse event of tumor flare (defined as local pain, irritation, or rash localized at sites of known or suspected tumor) that resolves to Grade ≤2 in ≤7 days
   Grade 3 laboratory abnormalities that are asymptomatic and considered by the investigator not to be clinically significant
   Grade 3 rash that resolves to Grade ≤2 in ≤7 days with therapy equivalent to prednisone 10 mg/day or less
   Grade 3 arthralgia that can be adequately managed with supportive care or that resolves to Grade ≤2 within 7 days
   Grade 3 autoimmune thyroiditis or other endocrine abnormality that can be managed by endocrine therapy that would not necessitate initiation of systemic corticosteroids (with the exception of replacement steroids for adrenal insufficiency)
Grade ≥4 neutropenia (absolute neutrophil count (ANC)<500/μL) lasting >7 days
Grade ≥3 febrile neutropenia
Grade ≥4 anemia
Grade ≥4 thrombocytopenia, or Grade 3 thrombocytopenia associated with clinically significant bleeding
Grade ≥3 elevation of serum hepatic transaminase (ALT or AST) lasting >7 days
   For patients with Grade 1 ALT or AST elevation at baseline as a result of liver metastases, only Grade ≥3 elevation that is also ≥3×baseline lasting >7 days is considered a DLT.
Grade ≥3 elevation of serum total bilirubin
   ALT or AST ≥3×upper limit of normal (ULN) AND total bilirubin >2×ULN

Dose-Escalation Rules

Enrollment begins in the Phase Ia portion of the study first. The starting dose of tiragolumab is 2 mg, administered IV, every 21 days. The dose of tiragolumab is increased ≤4-fold between successive dose levels, and the proposed approximate dose levels for evaluation for tiragolumab are 2 mg, 8 mg, 30 mg, 100 mg, 400 mg, and 1200 mg. The maximum dose increase between successive dose levels may be decreased. On the basis of emerging nonclinical efficacy, clinical safety, and/or clinical PK data, additional intermediate dose levels of tiragolumab may be evaluated.

In addition to any DLTs, other available relevant demographic, adverse event, laboratory, dose administration, PK (if available), and pharmacodynamic (if available) data are reviewed prior to dose-escalation decisions, which are made by the Medical Monitor in consultation with the Principal Investigators and a committee composed of but not limited to the following: safety scientist, statistician, PK scientist, and/or clinical trial manager.

Dose escalation occurs in accordance with the rules listed below.
A minimum of 3 patients are enrolled at each dose level (cohort).
If none of the first 3 DLT-evaluable patients experiences a DLT during the DLT assessment window, enrollment of the next cohort at an increased dose level determined as described above may proceed.
If 1 of the first 3 DLT-evaluable patients in a given cohort experiences a DLT, then at least 3 additional patients are enrolled at the same dose level (e.g., cohort is expanded to 6 patients). If less than one-third of the evaluable patients experience a DLT (e.g., <2 of 6 patients), dose escalation continues, and enrollment of the next cohort at an increased dose level may proceed.
If a DLT is observed in at least one-third of the DLT-evaluable patients per dose level in a cohort (e.g., ≥2 of 6 patients), the MTD is exceeded, and dose escalation stops. The preceding cohort is expanded to a minimum of 6 patients, unless 6 patients have already been evaluated at that level. However, if the dose level at which the MTD is exceeded is ≥2-fold higher than the preceding dose level, 6 patients may be evaluated at an intermediate dose level.
If the MTD is exceeded at any dose level, the highest dose at which fewer than one-third of the DLT-evaluable patients (i.e., <2 of 6 patients) experience a DLT is declared the MTD.
If the MTD is not exceeded at any dose level, the highest dose administered in this study is declared the MAD.
Any dose level that has been shown not to exceed the MTD may be expanded if warranted based on investigator evaluation of non-DLT adverse events, including events occurring after Cycle 1 and events observed in the expansion cohorts.

On the basis of a review of available preliminary real-time safety data and available preliminary PK data (collected from either the Phase Ia and/or Phase Ib studies), dose escalation in Phase Ia and/or Phase Ib may be halted or limited as deemed appropriate.

After DLT assessment has been completed for the first two dosing cohorts in the Phase Ia portion of the study, safety data are reviewed with the investigators and by the IMC, which make a recommendation whether to activate the Phase Ib portion of the study. If it is deemed appropriate to activate Phase Ib, enrollment may then proceed in both portions of the study concurrently. The starting dose of tiragolumab in combination with atezolizumab is 2 mg, and atezolizumab is administered at a fixed dose of 1200 mg IV, with both drugs being administered every 21 days. At no time does the dose of tiragolumab in the Phase Ib portion of the study exceed the highest dose that has been shown not to exceed the MTD in the Phase Ia portion of the study. If the combination MTD is exceeded in Cohort 1 in the Phase Ib portion of the study, a lower dose of tiragolumab may be considered in combination with atezolizumab.

To acquire additional safety and pharmacodynamic data to more fully inform the dose selection for the expansion cohorts, additional patients may be enrolled at dose levels in the Phase Ia and/or Phase Ib portions of the study that have been shown to not exceed the MTD or MAD based on the dose-escalation criteria described above. For the purposes of dose-escalation decisions, these patients are not considered part of the DLT-evaluable population. These patients must have tumors that are safely accessible and must consent to a biopsy. In order to inform the assessment of pharmacodynamics, patients with tumors expressing PD-L1 and/or TIGIT based on prospective testing of tumor tissue during screening or prescreening are enrolled. Furthermore, up to approximately 12 additional patients may be enrolled at a selected dose level in the Phase Ia or Phase Ib studies at or below the MTD or MAD in order to provide additional safety data prior to beginning the dose-expansion stage.

In the Phase Ia portion of the study only, patients who have developed radiographic progression and/or who are no longer deriving clinical benefit from single-agent tiragolumab may be eligible to receive treatment on the Phase Ib portion of the study provided that they have safely accessible tumors, consent to a biopsy, and meet the specified criteria for crossover.

Expansion Stage for Phase Ia and Phase Ib

Patients with locally advanced, recurrent, or metastatic incurable malignancies that have progressed after available standard therapy; or for whom standard therapy has proven to be ineffective or intolerable, or is considered inappropriate; or for whom a clinical trial of an investigational agent is a recognized standard of care, are enrolled in the expansion cohorts of the study. For the Phase Ib portion of the study only, patients for whom a clinical trial of an investigational agent in combination with an anti-PD-L1/PD-1 antibody with or without chemotherapy or anti-VEGF antibody is considered an acceptable treatment option may be enrolled in the expansion cohorts.

This expansion stage consists of defined cohorts of patients to better characterize the safety, tolerability, PK variability, pharmacodynamic activity, and preliminary anti-tumor activity of tiragolumab as a single agent (Phase Ia) or in combination with the following: atezolizumab, atezolizumab with chemotherapy, atezolizumab with bevacizumab, or pembrolizumab (Phase Ib) in specific cancer settings. Enrollment in the expansion cohorts is initiated at a selected dose level at or below the MAD or MTD of tiragolumab as a single agent (Phase Ia) or tiragolumab in combination with the following: atezolizumab, atezolizumab with chemotherapy, atezolizumab with bevacizumab, or pembrolizumab (Phase Ib) based on an assessment of accumulating safety, tolerability, PK, pharmacodynamic, and anti-tumor activity data.

In the Phase Ia portion of the study, up to approximately 40 patients are enrolled in a planned expansion cohort of multiple tumor indications that are PD-L1-selected and/or TIGIT-selected, including NSCLC, RCC, TNBC, melanoma, HNSCC, OC, GC including GEJ cancer, UBC, and CRC, including CRC that is MSS or MSI-Low.

In the Phase Ib portion of the study (without chemotherapy), approximately 20-40 patients may be enrolled in multiple tumor indications, including each of the following planned indication-specific expansion cohorts:
NSCLC: Cancer immunotherapy (CIT)-Naive (e.g., no prior treatment with anti-PD-L1/PD-1)
NSCLC: CIT-Treated (e.g., including prior treatment with anti-PD-L1/PD-1)
RCC
TNBC
Melanoma
HNSCC
OC
GC including GEJ cancer
UBC
CRC, including CRC that is MSS or MSI-Low
Biopsy cohort of specific tumor indications, including melanoma, OC, RCC, and UBC
   In the Phase Ib chemotherapy expansion portion of the study, approximately 20-40 patients may be enrolled in each of the following planned chemotherapy expansion cohorts (see FIG. 5):
Cohort A:
   Induction phase—atezolizumab and tiragolumab in combination with cisplatin or carboplatin and pemetrexed
   Maintenance phase—atezolizumab and tiragolumab in combination with pemetrexed
Cohort B:
   Induction phase—atezolizumab and tiragolumab in combination with carboplatin and paclitaxel
   Maintenance phase—atezolizumab and tiragolumab
Cohort C:
   Induction phase—atezolizumab and tiragolumab in combination with cisplatin or carboplatin and etoposide
   Maintenance phase—atezolizumab and tiragolumab
Cohort D:
   Atezolizumab and tiragolumab in combination with capecitabine
      In the Phase Ib Q4W dosing-expansion portion of the study, approximately 20-40 patients may be enrolled in the planned cohort (see FIG. 4).
      In the Phase Ib non-chemotherapy expansion portion of the study, approximately 20-40 patients may be enrolled in each of the following planned non-chemotherapy expansion cohorts (see FIG. 6).
Cohort NC1:
   Tiragolumab and atezolizumab with bevacizumab
Cohort NC2:
   Tiragolumab and pembrolizumab

CIT-naive patients for whom a clinical trial of an investigational agent in combination with an anti-PD-L1 antibody is considered an acceptable treatment option are only considered eligible for enrollment in the relevant expansion cohorts of the Phase Ib portion of the study. Otherwise, patients are assigned to available expansion slots in the Phase Ia or Phase Ib portions of the study based on investigator discretion.

Given that the safety profile of tiragolumab as a single agent (Phase Ia) or tiragolumab in combination with atezolizumab (Phase Ib) may not be fully characterized following evaluation of patients in the dose-escalation stage, not all of the potential adverse events or the likelihood of their occurrence can be known at the time of enrollment in the expansion cohorts. All available safety data are evaluated on an ongoing basis to assess the tolerability of the dose levels studied. To further mitigate the risk of potential unexpected adverse events, the IMC reviews the safety data from all patients entered in the expansion cohorts of the Phase Ia and Phase Ib portions of the study on a regular basis and make recommendations regarding study conduct related to patient safety. If the frequency of Grade 3 or Grade 4 toxicities or other unacceptable toxicities observed in an expansion-stage cohort suggest that the MTD has been exceeded at that dose level in Phase Ia or Phase Ib, accrual at that dose level is halted. Consideration is then given to resuming enrollment in the expansion stage at a lower dose level. In addition, if accumulating safety, tolerability, PK, or pharmacodynamic data suggest that the dose level in an expansion cohort is suboptimal for the evaluation of preliminary anti-tumor activity of tiragolumab as a single agent (Phase Ia) or tiragolumab in combination with atezolizumab (Phase Ib), consideration is given to enroll new patients in that cohort to a different dose level. At no time does a dose level studied in the expansion stage of Phase Ia or Phase Ib exceed the highest dose level that has been shown not to exceed the MTD in the respective dose-escalation stage of Phase Ia or Phase Ib.

In the Phase Ib chemotherapy expansion cohorts, Phase Ib Q4W dosing expansion cohort, and Phase Ib non-chemotherapy expansion cohorts, the IMC also monitors initial safety after the first 3 patients in each cohort have all completed 1 cycle of study treatment during the safety run-in (see FIG. 7). The IMC reviews all safety data in the first 3 patients for that cohort to determine whether study treatment is tolerable, in which there is an acceptable assessment of risk by the IMC and by the investigators to allow patients to continue dosing. If the study treatment is deemed tolerable in the safety run-in in each cohort, enrollment may continue at the same dose of tiragolumab in each cohort until the interim analysis for safety. This occurs after approximately 20 patients are enrolled, including patients in the safety run-in.

The IMC also monitors efficacy in the expansion cohorts in the Phase Ia and Phase Ib portions of the study by conducting an interim analysis after approximately 20 patients in each cohort have completed the first tumor assessment. If anti-tumor activity and/or clinical benefit per investigator is not detected in that expansion cohort, enrollment may be halted as decided by the IMC in consultation with study investigators. If anti-tumor activity and/or clinical benefit per investigator is detected in patients in that expansion cohort, the IMC may allow enrollment to continue to approximately 40 patients. There is no increase in the sample size of any expansion cohort to beyond 40 evaluable patients. Patients who had consented to serial biopsies but do not have evaluable samples may be replaced as part of the maximum 40 evaluable patients to be enrolled in each expansion cohort.

Phase Ia Expansion Cohort

The objectives for the Phase Ia expansion cohort are to better characterize the safety, tolerability, PK, and preliminary efficacy data and to explore potential tumor biomarkers of pharmacodynamic activity in patients treated with tiragolumab at a dose level equal to or less than its single-agent MTD or MAD. This cohort consists of approximately 20-40 patients with tumors that are PD-L1-selected and/or TIGIT-selected. Enrollment is initially limited to patients with the following tumor types: NSCLC, RCC, TNBC, melanoma, HNSCC, OC, GC including GEJ cancer, UBC, and CRC including MSS and MSI-Low. If anti-tumor activity and/or clinical benefit per the investigator is observed in patients with other tumor types on this study, enrollment may be expanded and/or modified to include other indications.

Up to approximately half of the patients enrolled in this cohort in Phase Ia must have safely accessible tumor lesions and consent to pre-treatment and on-treatment biopsies (core needle, punch, forceps, or excisional/incisional). Additional patients may be enrolled to ensure that approximately half of the patients in this cohort have evaluable serial biopsies with sufficient viable tumor content.

Patients who have developed radiographic progression and/or who are no longer deriving clinical benefit from single-agent tiragolumab in this Phase Ia expansion cohort may be eligible to crossover and receive treatment on the Phase Ib portion of the study (FIG. 8) provided that patients have safely accessible tumors, consent to a biopsy, and meet the specified criteria for crossover.

Phase Ib Indication-Specific Expansion Cohorts

The objectives of the Phase Ib indication-specific expansion cohorts are to better characterize the safety, tolerability, PK, and preliminary efficacy data and to explore potential tumor biomarkers of pharmacodynamic activity in patients with specific cancer types treated with tiragolumab at a dose level equal to or less than its MTD or MAD in combination with atezolizumab. Each indication-specific expansion cohort initially enrolls up to approximately 20 patients with tumors that are PD-L1-selected and/or TIGIT-selected based on prospective testing of tumor tissue during screening or prescreening and that include multiple tumor indications, including the following tumor types:
NSCLC: CIT-naive
These patients have not received prior CIT (investigational or approved), including anti-PD-L1/PD-1 and/or anti-CTLA-4, and may only be enrolled if a clinical trial of an investigational agent in combination with an anti-PD-L1 antibody is considered an acceptable treatment option.
NSCLC: CIT-treated
   These patients have received prior CIT (investigational or approved), including anti-PD-L1/PD-1.
TNBC
RCC
CRC
Gastric cancer (GC), including gastroesophageal junction (GEJ) cancer
HNSCC
UBC
Melanoma
OC

Following the enrollment of 20 patients, the IMC meets to conduct an interim analysis to determine if there is evidence of anti-tumor activity and/or clinical benefit as assessed by the investigators in order to continue enrollment to approximately 40 patients.

Up to approximately half of the patients enrolled in each of these indication-specific expansion cohorts in Phase Ib must have safely accessible tumor lesions and consent to pre-treatment and on-treatment biopsies (for example, core needle, punch, forceps, or excisional/incisional biopsies). Additional patients may be enrolled to ensure that approximately half of the patients in this cohort have evaluable serial biopsies with sufficient viable tumor content.

Phase Ib Serial Biopsy Expansion Cohort

The objectives of the Phase Ib serial biopsy expansion cohort are to explore potential tumor biomarkers of pharmacodynamic activity and to better characterize the safety, tolerability, PK, and preliminary efficacy data in patients with specific cancer types treated with a dose level of tiragolumab that is at or less than the MTD or MAD in combination with atezolizumab. The cohort consists of approximately 20-40 patients with tumors that are PD-L1-selected and/or TIGIT-selected based on prospective testing of tumor tissue during screening or prescreening and that may include the following tumor types:
Melanoma
RCC
OC
UBC

Patients enrolled in this serial biopsy expansion cohort must have safely accessible tumor lesions as they are required to undergo two tumor biopsies: 1) pre-treatment biopsy: at baseline after all eligibility criteria (other than the requirement for available archival tissue) have been fulfilled and prior to dosing with study drugs, and 2) on-treatment biopsy: at approximately 2 weeks after the first administration of tiragolumab in combination with atezolizumab (on or between Days 15-21 of Cycle 1). RECIST target lesions are not biopsied.

Tissue biopsy methods may include core needle, punch, forceps, or excisional/incisional biopsies. Patients who provide required fresh biopsies are to also submit archival tumor specimens, if available.

In this serial biopsy cohort, a recent archival specimen may be used in place of the fresh baseline biopsy under the following circumstances:
The specimen meets the sample criteria (e.g., number of cores or size of punch).
The specimen was collected within 3 months, or within an interval approved by the Medical Monitor, of the proposed Cycle 1, Day 1.
The specimen was collected subsequent to the most recent systemic therapy and/or to any radiation therapy administered to the relevant anatomic region.
The specimen originates from the same lesion or organ as the proposed site of the on-treatment biopsy.

Patients whose baseline biopsy is found to be non-evaluable (i.e., due to insufficient material or lack of tumor cells in the sample) still receive study treatment and are considered for an on-treatment biopsy. Such patients, as well as patients whose on-treatment biopsy is found to be non-evaluable or any patients with biopsies without sufficient viable tumor content, may be replaced for the purpose of serial biopsy assessment. Additional optional biopsies may be collected at the investigator's discretion and with the consent of the patient, preferably at the time of radiographic response or progression.

Optional Biopsies in Expansion Cohorts

Patients who are enrolled in expansion cohorts are asked to consider optional biopsies (for example, core needle, punch, forceps, or excisional/incisional biopsies) to explore pharmacodynamic changes related to the activity of tiragolumab as a single agent or in combination with atezolizumab. Enrollment of each of the expansion cohorts is managed so that up to approximately half of the accrued patients are those who have safely accessible tumor lesions and who consent to undergo these optional biopsies. The recommended biopsy timepoints under this scenario are the same as described above: at baseline after all eligibility criteria (other than the requirement for available archival tissue) have been fulfilled, and approximately 2 weeks after the first administration of tiragolumab (on or between Days 15-21 of Cycle 1).

On-treatment biopsies are not required if the baseline sample is non-evaluable and no recent archival specimen is available for comparison. Patients with a non-evaluable baseline or on-treatment biopsy (i.e., biopsy has insufficient viable tumor content) may be replaced for the purpose of serial biopsy assessment.

Patients who provide optional baselines biopsies are to also submit archival tumor specimens, if available.

Phase Ib Chemotherapy Expansion Cohorts

The objectives of the Phase Ib chemotherapy expansion cohorts are to better characterize the safety, tolerability, pharmacokinetics, and preliminary efficacy data and to explore potential tumor biomarkers of pharmacodynamic activity in patients treated with tiragolumab in combination with atezolizumab and chemotherapy.

Each cohort consists of approximately 20-40 patients with tumors that may be PD-L1-selected and/or TIGIT-selected based on prospective testing of tumor tissue during screening or prescreening. A patient with insufficient or unavailable archival tissue may be eligible for enrollment in this cohort, if deemed so by the Medical Monitor based upon a discussion with the investigator. Patients with a tumor type for whom a clinical trial of an investigational agent in combination with an anti-PD-L1 antibody with chemotherapy is considered an acceptable treatment option may be enrolled in these expansion cohorts. The number of patients with particular tumor types (e.g., NSCLC, SCLC, TNBC) or patients with specific treatment histories (e.g., CIT-naive) may be limited in each study cohort.

The treatment combinations in each cohort are shown in Table 29.

TABLE 29
Treatment Regimens in the Chemotherapy Expansion Cohorts

| | | Maintenance Phase |
|---|---|---|
| Treatment | Induction Phase | (21-day Cycles ^{a} |
| Cohort | (Four or Six 21-day Cycles) | or 28-day Cycles ^{b}) |
| A | Atezolizumab + tiragolumab + | Atezolizumab + |
| | pemetrexed + cisplatin or carboplatin | tiragolumab + |
| | | pemetrexed |
| B | Atezolizumab + tiragolumab + | Atezolizumab + |
| | paclitaxel + carboplatin | tiragolumab |
| C | Atezolizumab + tiragolumab + | Atezolizumab + |
| | cisplatin or carboplatin + etoposide | tiragolumab |
| D | Atezolizumab + tiragolumab + | |
| | capecitabine | |

^{a} For patients in Cohort A, Cohort B with Q3W maintenance (enrolled under protocol GO30103 Version 4) and for Cohort D.
^{b} For patients in Cohort B with Q4W maintenance (enrolled under protocol GO30103 Version 5 or later) and for Cohort C.

Each cohort has an initial safety run-in of 3 patients (see FIG. 5). After the first 3 patients in a cohort have completed 21 days of study treatment (i.e., Days 1-21 of Cycle 1), the IMC reviews all safety data for that cohort to determine whether the study treatment is tolerable, in which there is an acceptable assessment of risk by the IMC and by the investigators to allow patients to continue dosing of atezolizumab and tiragolumab and chemotherapy. Any patient who does not complete the first 21 days of treatment for a reason other than treatment-related toxicity may be replaced by an additional patient at the same dose level. If study treatment is deemed tolerable in the safety run-in for a cohort by the IMC after a minimum of 3 patients have enrolled and have completed 21 days of study treatment, enrollment may continue in that cohort until a total of approximately 20 patients are enrolled. An IMC reviews the efficacy data for the first approximately 20 patients in each cohort to evaluate safety and efficacy. If the IMC determines there is favorable benefit-risk, enrollment in that cohort may continue, up to a total of approximately 40 patients.

If the 600 mg dose of tiragolumab is not tolerated during the safety run-in in any cohort, a safety run-in at a lower dose of tiragolumab may be initiated for 3 more patients enrolled in that cohort. The safety data for this lower dose group are evaluated after the first 3 patients in that cohort have completed 21 days of study treatment (Days 1-21 of Cycle 1). Approximately 3 additional patients in the safety run-in would then be enrolled to further assess safety and tolerability of tiragolumab at this lower dose. Only the dose of tiragolumab may be reduced; the dose of atezolizumab does not change. Dose adjustments of chemotherapy, if necessary, follow the guidelines below. If study treatment at this lower dose is tolerated, enrollment continues at the lower dose until a total of approximately 20 patients are enrolled in each cohort, including patients in the lower dose safety run-in. An IMC reviews the data for the 20 patients in each cohort to evaluate safety and efficacy. If the IMC determines there is favorable benefit-risk, enrollment in that cohort may continue, up to a total of approximately 40 patients at the lower dose.

If the 600 mg dose of tiragolumab is well tolerated during the safety run-in, and emerging clinical PK data (e.g., PK data differ with chemotherapy) and/or pharmacodynamic data (e.g., receptor occupancy) suggest a higher dose of tiragolumab is to be used, a safety run-in at a higher dose of tiragolumab is initiated for the next 3 patients enrolled in each cohort. This higher dose does not exceed 1200 mg every 3 weeks, which is the maximum assessed dose for MTG7192A in both the Phase Ia and Phase Ib portions of this study, and does not have a maximum concentration (Cₘₐₓ) observed greater than what was observed at the 1200 mg every-3-weeks dose level. If study treatment at this higher dose of tiragolumab is tolerated, enrollment continues in the safety run-in at the higher dose until a minimum of 6 patients have enrolled and have completed 21 days of study treatment. If study treatment at this higher dose is deemed tolerable in the safety run-in by the IMC, enrollment may continue at this higher dose of tiragolumab until a total of approximately 20 patients are enrolled in each cohort. An IMC reviews the data for the first 20 patients in each cohort to evaluate safety and efficacy. If the IMC determines there is a favorable benefit-risk profile, enrollment in that cohort may continue, up to a total of approximately 40 patients at the higher dose.

Phase Ib Chemotherapy Expansion Cohorts: Dosing and Administration

Patients in Cohorts A, B, and C may receive treatment in 2 phases: an induction phase and a maintenance phase. In the induction phase, patients in the specific chemotherapy expansion cohorts receive the following:
Cohort A receives atezolizumab 1200 mg IV, then tiragolumab 600 mg IV, followed by the pemetrexed 500 mg/m² IV, followed by the cisplatin 75 mg/m² IV or carboplatin AUC of 6 mg/mL min IV on Day 1 of an every 21-day cycle. Four to six cycles of induction-phase treatment is administered in the absence of disease progression or unacceptable toxicity.
Cohort B receives atezolizumab 1200 mg IV, then tiragolumab 600 mg IV, followed by the paclitaxel 200 mg/m² IV, followed by the carboplatin AUC of 6 mg/mL min IV on Day 1 of an every 21-day cycle. Four to six cycles of induction phase treatment are administered in the absence of disease progression or unacceptable toxicity.
Cohort C receives atezolizumab 1200 mg IV, then tiragolumab 600 mg IV followed by cisplatin 75 mg/m² IV or carboplatin AUC of 5 mg/mL min IV on Day 1 of an every 21-day cycle and then etoposide 100 mg/m² IV on Days 1-3 of an every 21-day cycle. Four cycles of induction phase treatment are administered in the absence of disease progression or unacceptable toxicity.

For Cohort A and C, the choice of cisplatin or carboplatin is the investigator's decision, although the number of patients that receive a particular platinum-based chemotherapeutic agent may be limited. For Cohorts A and B, the number of cycles administered in the induction phase (four to six) is the investigator's decision.

Following the induction phase, treatment may continue in the maintenance phase in the absence of unacceptable toxicity, clinically compelling disease progression, and/or loss of clinical benefit at the investigator's discretion following a careful assessment and thorough discussion of the potential risks and benefits with the patient.

In the maintenance phase, study treatment continues every Q3W for Cohort A, Cohort B enrolled under protocol Version 4, and Cohort D, or continues every Q4W for Cohort B enrolled under protocol Version 5 or later and Cohort C.

In the maintenance phase, patients in the specific chemotherapy expansion cohorts receive the following:
Cohort A receives atezolizumab 1200 mg IV, then tiragolumab 600 mg IV, followed by pemetrexed 500 mg/m² IV on Day 1 of an every-21-day cycle
Cohort B receives atezolizumab 1680 mg IV and then tiragolumab 840 mg IV on Day 1 of an every-28-day cycle. If patients were enrolled under GO30103 Version 4, patients in Cohort B continue to receive atezolizumab 1200 mg IV and then tiragolumab 600 mg IV on Day 1 of an every-21-day cycle
Cohort C receives atezolizumab 1680 mg IV and then tiragolumab 840 mg IV on Day 1 of an every-28-day cycle

Patients in Cohort D receive treatment with capecitabine, atezolizumab, and tiragolumab, until unacceptable toxicity or loss of clinical benefit as determined by the investigator. Patients receive capecitabine at a dose of 1250 mg/m² BID orally on Days 1 through 14 of each 21-day cycle. On Day 1 of Cycle 1, the first dose of capecitabine is administered at the clinic prior to the atezolizumab and tiragolumab infusion. The combination of atezolizumab and tiragolumab is administered by IV infusion on a 21-day cycle.

In the event of toxicity and the absence of disease progression or loss of clinical benefit, individual chemotherapy or immunotherapy agents may be independently discontinued. However, treatment with atezolizumab alone or tiragolumab alone (with or without chemotherapy) can only be considered if there is no contraindication and after discussion with the Medical Monitor.

Phase Ib 04W Dosing Expansion Cohort

The objectives of the Phase Ib Q4W dosing expansion cohort are to better characterize the safety, tolerability, PK, and preliminary efficacy data and to explore potential tumor biomarkers of pharmacodynamic activity in patients treated with tiragolumab 840 mg IV in combination with atezolizumab 1680 mg IV with an every-4-week (28 day; Q4W) dosing schedule.

The Phase Ib Q4W cohort consists of approximately 20-40 patients with tumors that may be PD-L1-selected and/or TIGIT-selected based on prospective testing of tumor tissue during screening or prescreening. A patient with insufficient or unavailable archival tissue may be eligible for enrollment in this cohort, if deemed so by the Medical Monitor based upon a discussion with the investigator. Patients with a tumor type for whom a clinical trial of an investigational agent in combination with an anti-PD-L1 antibody is considered an acceptable treatment option may be enrolled in these expansion cohorts. The number of patients with particular tumor types (e.g., NSCLC, SCLC) or patients with specific treatment histories (e.g., CIT-naive) may be limited in each study cohort.

This cohort has an initial safety run-in of 3 patients (see FIG. 4). After the first 3 patients in a cohort have completed 28 days of study treatment (i.e., Days 1-28 of Cycle 1), the IMC reviews all safety data for that cohort to determine whether the study treatment is tolerable, in which there is an acceptable assessment of risk by the IMC and by the investigators to allow patients to continue dosing of tiragolumab with atezolizumab. Any patient who does not complete the first 28 days of treatment for a reason other than treatment-related toxicity may be replaced by an additional patient at the same dose level. If study treatment is deemed tolerable in the safety run-in for a cohort by the IMC after a minimum of 3 patients have enrolled and have completed 28 days of study treatment, enrollment may continue in that cohort until a total of approximately 20 patients are enrolled. An IMC reviews the efficacy data for the first approximately 20 patients in each cohort to evaluate safety and efficacy. If the IMC determines there is favorable benefit-risk, enrollment in that cohort may continue, up to a total of approximately 40 patients.

If the 840 mg dose of tiragolumab is not tolerated during the safety run-in in any cohort, a safety run-in at a lower dose of tiragolumab may be initiated for 3 more patients enrolled in that cohort. The safety data for this lower dose group are evaluated after the first 3 patients in that cohort have completed 28 days of study treatment (Days 1-28 of Cycle 1). Approximately 3 additional patients are to be enrolled in the safety run-in to further assess safety and tolerability of tiragolumab at this lower dose. Only the dose of tiragolumab may be reduced; the dose of atezolizumab does not change. If study treatment at this lower dose is tolerated, enrollment continues at the lower dose until a total of approximately 20 patients are enrolled in each cohort, including patients in the lower dose safety run-in. An IMC reviews the data for the 20 patients in each cohort to evaluate safety and efficacy. If the IMC determines there is favorable benefit-risk, enrollment in that cohort may continue, up to a total of approximately 40 patients at the lower dose.

If the 840 mg dose of tiragolumab is well tolerated during the safety run-in, and emerging clinical PK data and/or pharmacodynamic data (e.g., receptor occupancy) suggest a higher dose of tiragolumab is to be used, a safety run-in at a higher dose of tiragolumab is initiated for the next 3 patients enrolled in each cohort. This higher dose does not exceed 1200 mg, which is the maximum assessed dose for MTG7192A in both the Phase Ia and Phase Ib portions of this study, and does not have a maximum concentration (Cₘₐₓ) observed greater than what was observed at the 1200 mg every-3-weeks dose level. If study treatment at this higher dose of tiragolumab is tolerated, enrollment continues in the safety run-in at the higher dose until a minimum of 6 patients have enrolled and have completed 28 days of study treatment. If study treatment at this higher dose is deemed tolerable in the safety run-in by the IMC, enrollment may continue at this higher dose of tiragolumab until a total of approximately 20 patients are enrolled in each cohort. An IMC reviews the data for the first 20 patients in each cohort to evaluate safety and efficacy. If the IMC determines there is favorable benefit-risk, enrollment in that cohort continues, up to a total of approximately 40 patients at the higher dose.

Phase Ib Non-Chemotherapy Expansion Cohort

The objectives of the Phase Ib non-chemotherapy expansion cohorts are to better characterize the safety, tolerability, and preliminary efficacy data and to explore potential tumor biomarkers of pharmacodynamic activity in patients treated with tiragolumab and atezolizumab with bevacizumab, and tiragolumab in combination with pembrolizumab.

Each cohort consists of approximately 20-40 patients with tumors that may be PD-L1-selected and/or TIGIT-selected based on prospective testing of tumor tissue during screening or prescreening. A patient with insufficient or unavailable archival tissue may be eligible for enrollment in this cohort, if deemed so by the Medical Monitor based upon a discussion with the investigator. Patients with a tumor type for whom a clinical trial of an investigational agent in combination with anti-PD-L1/PD-1 with or without bevacizumab is considered an acceptable treatment option, per investigator judgement, may be enrolled in these expansion cohorts. The number of patients with particular tumor types (e.g., HCC, melanoma) or patients with specific treatment histories (e.g., CIT-naive) may be limited in each study cohort.

The treatment combinations in each cohort are shown in Table 30.

TABLE 30
Treatment Combinations by Cohort

| | Treatment Regimen^{a} |
|---|---|
| Treatment Cohort | (21-day Cycles) |
| Cohort NC1 | Tiragolumab + Atezolizumab + Bevacizumab |
| Cohort NC2 | Tiragolumab + Pembrolizumab |

^{a}In order of drug administration

Each cohort has an initial safety run-in of approximately 3 patients (FIG. 6). After the first 3 patients in a cohort have completed 3 weeks (21 days) of study treatment (i.e., Days 1-21 of Cycle 1), the IMC reviews all safety data for that cohort to determine whether the study treatment is tolerable, in which there is an acceptable assessment of risk by the IMC and by the investigators to allow patients to continue dosing of tiragolumab and atezolizumab with bevacizumab (Cohort NC1) or tiragolumab and pembrolizumab (Cohort NC2). Any patient who does not complete the first 3 weeks of treatment for a reason other than treatment-related toxicity may be replaced by an additional patient at the same dose level. If study treatment is deemed tolerable in the safety run-in for a cohort by the IMC after a minimum of 3 patients have enrolled and have completed 3 weeks of study treatment, enrollment may continue in that cohort until a total of approximately 20 patients are enrolled. An IMC reviews the efficacy data for the first approximately 20 patients in each cohort to evaluate safety and efficacy. If the IMC determines there is favorable benefit-risk, enrollment in that cohort may continue, up to a total of approximately 40 patients.

If the 600 mg dose of tiragolumab is not tolerated during the safety run-in in any cohort, a safety run-in at a lower dose of tiragolumab may be initiated for approximately 3 more patients enrolled in that cohort. The safety data for this lower dose group are evaluated after the first 3 patients in that cohort have completed 3 weeks of study treatment (Days 1-21 of Cycle 1). Approximately 3 additional patients in the safety run-in would then be enrolled to further assess safety and tolerability of tiragolumab at this lower dose. Only the dose of tiragolumab may be reduced; the dose of pembrolizumab or atezolizumab with bevacizumab does not change. If study treatment at this lower dose is tolerated, enrollment continues at the lower dose until a total of approximately 20 patients are enrolled in each cohort, including patients in the lower dose safety run-in. An IMC reviews the data for the 20 patients in each cohort to evaluate safety and efficacy. If the IMC determines there is favorable benefit-risk, enrollment in that cohort may continue, up to a total of approximately 40 patients at the lower dose.

If the 600 mg dose of tiragolumab is well tolerated during the safety run-in, a safety run-in at a higher dose of tiragolumab might be initiated for the next approximately 3 patients enrolled in each cohort. This higher dose does not exceed 1200 mg every 3 weeks, which is the maximum assessed dose for tiragolumab in both the Phase Ia and Phase Ib portions of this study, and the observed maximum concentration (Cₘₐₓ) is not to be greater than what was observed at the 1200 mg every 3 weeks dose level. If study treatment at this higher dose of tiragolumab is tolerated, enrollment continues in the safety run-in at the higher dose until a minimum of 6 patients have enrolled and have completed 3 weeks of study treatment. If study treatment at this higher dose is deemed tolerable in the safety run-in by the IMC, enrollment may continue at this higher dose of tiragolumab until a total of approximately 20 patients are enrolled in each cohort. An IMC reviews the data for the first 20 patients in each cohort to evaluate safety and efficacy. If the IMC determines there is a favorable benefit-risk profile, enrollment in that cohort may continue, up to a total of approximately 40 patients at the higher dose.

Dose Reduction of Tiragolumab and Atezolizumab and Other Non-Chemotherapy Agents

In general, there is no dose reduction for tiragolumab in this study. However, if available cumulative safety data suggest that a dose level of tiragolumab initially selected as a single agent (Phase Ia) or in combination with atezolizumab (Phase Ib) exceeds the MTD or MAD, accrual at that dose level may be halted and, if applicable, further dose escalation is halted.

In this circumstance, individual patients have the option of dose reduction to the new dose level of tiragolumab if the following criteria are met:
The patient's initially assigned dose is equal to (or greater than) the dose level that has been closed to further enrollment
The overall benefit/risk balance favors continued treatment in Phase Ia or Phase Ib, in the opinion of the investigator

For atezolizumab, there is no intrapatient dose reduction; atezolizumab is to be administered at a fixed dose of 1200 mg every 21 days or 1680 mg every 28 days (Q4W dosing expansion cohort).

For bevacizumab, there is no intrapatient dose reduction; bevacizumab is to be administered at a fixed dose of 15 mg/kg every 3 weeks.

For pembrolizumab, there is no intrapatient dose reduction; pembrolizumab is to be administered at a fixed dose of 200 mg every 3 weeks.

Treatment after Disease Progression

Patients may continue study treatment in the Phase Ia or Phase Ib portions of the study after standard RECIST v1.1 criteria for progressive disease are met at the investigator's discretion after discussion with the Medical Monitor, and provided that the patients meet all of the following criteria (see FIG. 7):
Absence of symptoms and signs (including worsening of laboratory values, e.g., new or worsening hypercalcemia) indicating unequivocal progression of disease
No decline in Eastern Cooperative Oncology Group (ECOG) performance status
Absence of tumor progression at critical anatomical sites that cannot be readily managed and stabilized by protocol-allowed medical interventions prior to repeat dosing. Critical anatomical sites include the CNS, central airway, the great vessels, and other organs or tissues where compromised function secondary to tumor progression would be expected to result acutely in severe and/or irreversible disability or death.

Patients must provide consent to acknowledge discussion with the treating investigator of the benefit-risk balance of continuing study treatment beyond radiographic progression.

If radiographic disease progression is confirmed at a subsequent tumor assessment in Phase Ia or Phase Ib, patients may be considered for continued study treatment at the investigator's discretion after discussion with the Medical Monitor, if they continue to meet the above criteria and have continued clinical benefit, as evidenced by at least one of the following:
Tumor shrinkage (at least 30% decrease in diameter from baseline) of one or more evaluable lesions
Improvement in one or more symptoms or signs attributable to the underlying cancer (e.g., decreased requirement for narcotics for pain, decreased dyspnea associated with pleural effusion, weight gain) as assessed by the investigator

In the Phase Ia portion of the study only, patients treated with single-agent tiragolumab who develop progressive disease and/or are no longer deriving clinical benefit, as outlined above, may be eligible to receive treatment on the Phase Ib portion of the study with tiragolumab in combination with atezolizumab provided that patients have safely accessible tumors, consent to a biopsy, and meet the specified criteria for crossover.

In the chemotherapy expansion Cohorts A, B, C, and D, treatment after disease progression must include immunotherapy (atezolizumab and/or tiragolumab) with or without chemotherapy, and cannot be with chemotherapy alone.

Crossover from Single-Agent Tiragolumab Treatment to the Combination of Tiragolumab with Atezolizumab Upon Progression

Patients treated with tiragolumab as a single agent in the Phase Ia portion of the study who develop progressive disease and/or who are no longer deriving clinical benefit may be eligible to receive treatment with tiragolumab in combination with atezolizumab in the Phase Ib portion of the study (see FIG. 8) provided that the following conditions are met:
Patients must have radiographically documented disease progression in the Phase Ia portion of the study, including a CT scan of the chest, abdomen, and pelvis.
Patients must not have experienced a toxicity requiring the discontinuation of tiragolumab treatment and must not have experienced toxicity during the last dose of study treatment that would preclude treatment with the combination of tiragolumab and atezolizumab in the Phase Ib study.
Patients must meet all the inclusion and exclusion criteria.
Any tiragolumab-related adverse events must have resolved to Grade ≤1 or to baseline grade on or before the first day of treatment in the Phase Ib portion of the study. Exceptions may be allowed after a careful assessment and discussion of the benefit-risk balance with the patient by the investigator(s) and with approval from the Medical Monitor.
Treatment with tiragolumab and atezolizumab after progression on single-agent tiragolumab must be considered acceptable as determined after a careful assessment and discussion of the benefit-risk balance with the patient by the investigator(s) and with approval from the Medical Monitor.

A tumor biopsy is required for patients with safely accessible site(s) of disease. This biopsy may occur at any time prior to dosing with tiragolumab and atezolizumab in the Phase Ib study. An optional on-treatment biopsy may also be collected on or between Days 15-21 in Cycle 1 following dosing with tiragolumab and atezolizumab. RECIST target lesions are not to be biopsied.

The dose and schedule of crossover treatment with tiragolumab in combination with atezolizumab in the Phase Ib study requires approval from the Medical Monitor. In general, a patient crossing over into the Phase Ib portion of the study receives tiragolumab at the highest dose that has already been shown not to exceed its MTD in combination with atezolizumab (FIG. 7). Therefore, the dose level of tiragolumab evaluated in the Phase Ib study may be different from the dose level evaluated in the same patient in the Phase Ia study. Patients who had modifications to the dose and/or schedule of tiragolumab as a single agent in the Phase Ia study continue to receive tiragolumab at the same dose and/or schedule in Phase Ib, so long as that dose of tiragolumab has been previously shown not to exceed the MTD for tiragolumab with atezolizumab.

Patients who cross over from the Phase Ia portion of the study to the Phase Ib portion of the study are treated as follows:
Assessments obtained at the study treatment discontinuation visit in Phase Ia may be used as screening assessments for combination treatment in the Phase Ib portion of the study. The following re-screening assessments must be repeated/obtained within 2 weeks prior to the first day of starting treatment on the combination regimen, in order to re-establish baseline pretreatment clinical and disease status: targeted physical examination, ECOG status, hematology and serum chemistry laboratory tests, coagulation panel, amylase, lipase, thyroid function tests, and urine analysis.
Screening tests for hepatitis B, hepatitis C, HIV, cytomegalovirus (CMV), and EBV do not need to be repeated unless there is a clinical suspicion for positivity of these test results since they were last conducted during screening for the Phase Ia study.
A radiographic tumor assessment, including a CT scan of the chest, abdomen, and pelvis, must also be performed, unless already done to document disease progression, within 6 weeks prior to the first day of starting combination treatment in the Phase Ib study, as this assessment becomes the new baseline scan for the patient enrolling on the Phase Ib study.
Patients are treated with the combination of tiragolumab and atezolizumab in the Phase Ib study until a second disease progression event relative to the tumor assessment that documented progressive disease on the initial study treatment, clinical deterioration, intolerance to combination treatment, and/or loss of clinical benefit from the combination treatment. As previously described, patients may continue study treatment after standard RECIST v1.1 criteria for progressive disease are met in the Phase Ib study at the investigator's discretion after discussion with the Medical Monitor, and provided, they meet all the criteria specified.
Patients who crossover are evaluated for safety and efficacy.

Clinical data and exploratory data derived from tumor biopsies obtained prior to crossover treatment from Phase Ia to Phase Ib are monitored on an ongoing basis.

Crossover enrollment into the Phase Ib study from the Phase Ia portion may be restricted or suspended at any time. Reasons for this may include safety, unsatisfactory patient enrollment, and/or incomplete data recording.

C. Study Treatment

Dosing and Administration

Administration of tiragolumab as a single agent (Phase Ia) or tiragolumab in combination with atezolizumab (Phase Ib) is performed in a setting with emergency medical facilities with access to a critical care unit and staff who are trained to monitor for and respond to medical emergencies.

For treatment in the Phase Ib portion of the study (cohorts without chemotherapy), on days of scheduled study treatment infusion, tiragolumab is administered prior to atezolizumab, with an intervening observation period.

For treatment in the Phase Ib chemotherapy expansion cohorts, on days of scheduled study treatment infusion, atezolizumab is administered prior to tiragolumab, with an intervening observation period. Chemotherapy is administered after atezolizumab and tiragolumab, with an intervening observation period for tiragolumab.

For treatment in the Phase Ib non-chemotherapy expansion cohorts, on days of scheduled study treatment infusion, tiragolumab is administered prior to atezolizumab with bevacizumab (Cohort NC1) or pembrolizumab (Cohort NC2), with an intervening observation period. Bevacizumab is administered after tiragolumab and atezolizumab, with an intervening observation period for bevacizumab.

For details on dose preparation and administration instructions for tiragolumab, atezolizumab, or bevacizumab refer to the respective Investigator's Brochures and to the GO301 03 Pharmacy Manual.

For details on dose preparation and administration instructions for cisplatin, carboplatin, pemetrexed, paclitaxel, capecitabine, etoposide, and pembrolizumab refer to the Package Inserts or SmPC.

Tiragolumab in Phase Ia and Phase Ib

The approximate dose levels of tiragolumab to be evaluated in the Phase Ia and Phase Ib portions of this study are 2 mg, 8 mg, 30 mg, 100 mg, 400 mg, and 1200 mg administered every 3 weeks by IV infusion. Other dose levels that do not exceed the MTD in Phase Ia or Phase Ib, or new dosing schedules of tiragolumab in Phase Ia and/or Phase Ib may be evaluated based on new nonclinical efficacy, clinical safety, and clinical PK data after consultation with participating investigators. Evaluation of dose levels exceeding tiragolumab 1200 mg require a protocol amendment with a supporting rationale. For the Phase Ib chemotherapy and non-chemotherapy expansion cohorts, tiragolumab 600 mg is the initial dose administered every 3 weeks by IV infusion. For the Phase Ib chemotherapy Cohort B with Q4W maintenance and Cohort C, the tiragolumab dose changes to 840 mg in maintenance. For the Phase Ib Q4W dosing cohort, 840 mg tiragolumab is the initial dose administered Q4W by IV infusion. Doses are not dependent on body weight.

The 840 mg Q4W dosing regimen is supported by results from PK modeling and simulation and exposure-safety analyses. The average concentration following the 840 mg Q4W dosing regimen is comparable to that of the 600 mg every 3 weeks dosing regimen, which was evaluated in Studies GO30103 and GO40290. The Cmax of the 840 mg Q4W dosing regimen is 28% higher at steady state, relative to the Cmax for the 600 mg every 3 weeks dosing regimen but falls within the range of observed exposure of the highest administered dose in the clinic (1200 mg every 3 weeks in Study GO301 03). A preliminary analysis of the tiragolumab exposure-safety relationship based on data from Study GO30103 (tiragolumab doses of 2-1200 mg every 3 weeks administered as monotherapy or in combination with atezolizumab 1200 mg every 3 weeks) suggest that tiragolumab demonstrates a flat exposure-safety relationship. In summary, the 840 mg Q4W dosing regimen has comparable safety and efficacy as the 600 mg every-3-weeks dosing regimen given that the exposure is within the range of observed efficacious exposures and tiragolumab appears to have a flat exposure-safety relationship.

The initial dose of tiragolumab in Phase Ia of the study is delivered over 90 (±10) minutes (although the infusion may be slowed or interrupted for patients who experience infusion-associated symptoms), followed by a 240-minute observation. The initial dose of tiragolumab in Phase Ib of the study is delivered over 60 (±10) minutes, followed by a 60-minute observation period. If the initial infusion is tolerated without infusion-associated adverse events, the second infusion in Phase Ia may be delivered over 60 (±10) minutes, followed by a 60-minute observation period and the second infusion in Phase Ib may be delivered over 30 (±10) minutes, followed by a 30-minutes observation period. If the 60-minute infusion in Phase Ia is well tolerated, the third infusion in Phase Ia may be delivered over 30 (±10) minutes, followed by a 30-minute observation period. If the third infusion in Phase Ia or the second infusion in Phase Ib is well tolerated, all subsequent infusions in Phase Ia or Phase Ib may be delivered over 30 (±10) minutes, followed by a 30-minute observation period. Patients who have previously received single-agent tiragolumab in Phase Ia and are now receiving tiragolumab in combination with atezolizumab in the Phase Ib may receive the initial dose in Phase Ib at the fastest rate that the patient previously tolerated.

There is no dose reduction for tiragolumab in this study (Phase Ia or Phase Ib) except as specified. Guidelines for dosage modification and treatment interruption, or discontinuation for tiragolumab, are provided. Guidance on study drug administration in the context of management of specific adverse events is provided.

Adverse events associated with an overdose or incorrect administration of study drug are recorded.

Atezolizumab in Phase Ib

The dose of atezolizumab to be administered in combination with tiragolumab in the Phase Ib portion of this study is 1200 mg IV every 3 weeks. For the Phase Ib chemotherapy Cohort B with Q4W maintenance and Cohort C, the atezolizumab dose changes to 1680 mg in maintenance. In the Phase Ib Q4W dosing cohort where atezolizumab 1680 mg IV Q4W is administered. This dose is fixed and not dependent on body weight.

In all Phase Ib cohorts without chemotherapy, atezolizumab is administered after the tiragolumab infusion and subsequent observation period. In the Phase Ib chemotherapy expansion cohorts, atezolizumab is administered before tiragolumab.

The initial dose of atezolizumab is delivered over 60 (±10) minutes. If the first infusion is tolerated without infusion-associated adverse events, the second infusion may be delivered over 30 (±10) minutes. If the 30-minute infusion is well tolerated, all subsequent infusions may be delivered over 30 (±10) minutes. For Cycle 1, dosing of atezolizumab is followed by a 60-minute observation period. All subsequent infusions of atezolizumab may be followed by a 30-minute observation period. Patients who have previously received atezolizumab on another clinical trial may receive the initial dose at the fastest rate that was previously tolerated.

There is no dose reduction for atezolizumab in this study. Guidelines for dosage modification and treatment interruption or discontinuation are provided. Guidance on study drug administration in the context of management of specific adverse events is provided.

Adverse events associated with an overdose or incorrect administration of study drug EW recorded.

Non-Chemotherapy Cohorts in the Phase Ib Expansion

Cohort NC1: Tiragolumab and Atezolizumab with Bevacizumab

Patients in the tiragolumab and atezolizumab and bevacizumab cohort receive treatment as outlined in Table 31 until unacceptable toxicity or loss of clinical benefit as determined by the investigator after an integrated assessment. It is recommended that treatment be initiated no later than 7 days after enrollment; however, the first dose of study treatment is not to occur within 7 days after a core biopsy or other surgical procedure.

TABLE 31
Treatment Regimen for Cohort NC1

| Cycle Length | Dose, Route, and Regimen^{a} |
|---|---|
| 3 weeks | 1. Tiragolumab: 600 mg IV infusion on Day 1 |
| | 2. Atezolizumab: 1200 mg IV infusion on Day 1 |
| | 3. Bevacizumab: 15 mg/kg IV infusion on Day 1b |

IV = intravenous
^{a}Drugs listed in order of administration. All drugs to be administered by IV infusion on Day 1 of each 21-day Cycle.
^{b}On Day 1 of Cycle 1, bevacizumab is administered 60 minutes after the completion of the atezolizumab infusion. If the patient experienced an infusion-related reaction during the previous drug infusion, the interval between subsequent infusions is 60 minutes. If the previous drug infusion was tolerated without an infusion-related reaction, the interval may be reduced to 30 minutes.

Cohort NC2: Tiragolumab and Pembrolizumab

Patients in the tiragolumab and pembrolizumab cohort receive treatment as outlined in Table 32 until unacceptable toxicity or loss of clinical benefit as determined by the investigator after an integrated assessment. It is recommended that treatment be initiated no later than 7 days after enrollment; however, the first dose of study treatment is not to occur within 7 days after a core biopsy or other surgical procedure.

TABLE 32
Treatment Regimen for Cohort NC2

| Cycle Length | Dose, Route, and Regimen^{a} |
|---|---|
| 3 weeks | 1. Tiragolumab: 600 mg IV infusion on Day 1^{b} |
| | 2. Pembrolizumab: 200 mg IV infusion on Day 1^{b} |

IV = intravenous
^{a}Drugs listed in order of administration. All drugs to be administered by IV infusion on Day 1 of each 21-day Cycle.
^{b}On Day 1 of Cycle 1, pembrolizumab is administered 60 minutes after the completion of the tiragolumab infusion. If the patient experienced an infusion-related reaction during the previous tiragolumab infusion, the interval between subsequent infusions is 60 minutes. If the previous tiragolumab infusion was given without premedication and was tolerated without an infusion-related reaction, the interval may be reduced to 30 minutes.

Phase Ib Non-Chemotherapy Expansion Cohorts: Dosing and Administration

Patients in Cohort NC1 receive tiragolumab 600 mg IV, then atezolizumab 1200 mg IV, and then bevacizumab 15 mg/kg IV on Day 1 of an every 3 weeks cycle. Treatment is administered until unacceptable toxicity or loss of clinical benefit as determined by the investigator. Patients in Cohort NC2 receive tiragolumab 600 mg IV and then pembrolizumab 200 mg IV on Day 1 of an every 3 weeks cycle. Treatment is administered until unacceptable toxicity or loss of clinical benefit as determined by the investigator.

In the event of toxicity and the loss of clinical benefit, individual immunotherapy agents may be independently discontinued. However, treatment with pembrolizumab or bevacizumab or atezolizumab or tiragolumab alone can only be considered if there is no contraindication and after discussion with the Medical Monitor.

Bevacizumab in the Phase Ib Expansion Cohorts

Bevacizumab is administered by IV infusion at a fixed dose of 15 mg/kg on Day 1 of each 21-day Cycle. Administration of bevacizumab is performed in a monitored setting where there is immediate access to trained personnel and adequate equipment and medicine to manage potentially serious reactions. Guidelines for dosage modification and treatment interruption or discontinuation because of toxicities are provided.

No premedication is permitted prior to the first bevacizumab infusion. If the patient experienced an IRR with any previous drug infusion, premedication with anti-histamines, anti-pyretics, and/or analgesics may be administered at the discretion of the investigator. Vital signs (pulse rate, respiratory rate, blood pressure, and temperature) are measured within 60 minutes prior to the infusion. The initial dose of bevacizumab is delivered over 90 (±10) minutes. If the first infusion is tolerated without infusion-associated adverse events, the second infusion may be delivered over 60 (±10) minutes. If the 60-minute infusion is well tolerated, all subsequent infusions may be delivered over 30 (±10) minutes. For Cycle 1, dosing of bevacizumab is followed by a 60-minute observation period. All subsequent infusions of bevacizumab may be followed by a 30-minute observation period. Patients who have previously received bevacizumab may receive the initial dose at the fastest rate that was previously tolerated. Patients are informed about the possibility of delayed post-infusion symptoms and instructed to contact their study physician if they develop such symptoms. Instructions on vital sign measurements are provided.

Pembrolizumab in the Phase Ib Expansion Cohort

The dose of pembrolizumab to be administered in combination with tiragolumab in the Phase Ib non-chemotherapy expansion cohort of this study is 200 mg IV every 3 weeks. This dose is fixed and not dependent on body weight.

Refer to the pembrolizumab prescribing information for guidance on dose preparation, storage, administration, and treatment interruption or discontinuation instructions for pembrolizumab.

Chemotherapy in the Phase Ib Expansion Cohorts

Chemotherapy (except capecitabine) is administered after the atezolizumab and tiragolumab infusions and subsequent observation periods.

During the induction phase, a chemotherapy cycle counts toward the prespecified number of induction chemotherapy cycles as long as at least one chemotherapy component has been administered at least once during a 21-day cycle. Cycles in which no chemotherapy component is given do not count toward the total number of induction chemotherapy cycles.

During treatment (induction or maintenance), patients who show evidence of clinical benefit are permitted to continue treatment after the RECIST v1.1 criteria for PD are met.

Patients receive anti-emetics and IV hydration for chemotherapy agents according to the local standard-of-care and manufacturer's instruction. However, because of the immunomodulatory effects of steroids, premedication with steroids is minimized to the extent that is clinically feasible.

Guidelines for dose modification and treatment interruption or discontinuation for chemotherapy are provided.

Cohort A: Atezolizumab Plus Tiragolumab Plus Carboplatin/Cisplatin Plus Pemetrexed

On Day 1 of each 21-day cycle, all eligible patients receive drug infusions in the following order:

Induction: Atezolizumab→tiragolumab→pemetrexed→carboplatin or cisplatin

After 4 to 6 cycles in the induction phase, patients begin maintenance therapy in the following order of administration:

Maintenance: Atezolizumab→tiragolumab→pemetrexed

Table 33 lists the suggested premedication for pemetrexed. Table 7 lists the suggested infusion times for treatment administration for pemetrexed and carboplatin or cisplatin during the induction phase and for pemetrexed during the maintenance phase.

TABLE 33
Premedication for Pemetrexed

| Premedication | Dose and Route | | Timing |
|---|---|---|---|
| Folic acid | 350-1000 | μg PO | Once daily, beginning at least |
| | | | 5-7 days before Cycle 1, Day 1 |
| | | | and continuing until 3 weeks after |
| | | | discontinuation of pemetrexed |
| Vitamin B12 | 1000 | μg IM | Q9W, beginning Cycle 1, |
| | | | Day 1 and continuing until |
| | | | 3 weeks after discontinuation |
| | | | of pemetrexed |
| Dexamethasone | 4 | mg PO | BID, the day before, the day |
| (suggested) | | | of, and the day after |
| | | | pemetrexed administration |

BID = twice a day;
IM = intramuscular;
PO = oral;
Q9W = every 9 weeks.

Cohort B: Atezolizumab Plus Tiragolumab Plus Carboplatin Plus Paclitaxel

On Day 1 of each 21-day cycle, all eligible patients receive drug infusions in the following order:

Induction: Atezolizumab→tiragolumab→paclitaxel→carboplatin

After 4 to 6 cycles in the induction phase, patients begin maintenance therapy on Day 1 of each 21-day cycle if they enrolled under protocol Version 4, or on Day 1 of each 28-day cycle if they enrolled under protocol Version 5 or later. All eligible patients receive drug infusion in the following order of administration:

Maintenance: Atezolizumab→tiragolumab

Table 8 lists the suggested premedication for induction treatment for patients in Cohort B. Table 9 lists the suggested infusion times for treatment administration for paclitaxel and carboplatin during the induction phase.

Cohort C: Atezolizumab Plus Tiragolumab Plus Carboplatin or Cisplatin

On Day 1 of each 21-day cycle, all eligible patients are administered study drug infusions in the following order:

Induction: Atezolizumab→tiragolumab→cisplatin or carboplatin→etoposide

After the induction phase, patients begin maintenance therapy on Day1 of each 28-day cycle in the following order of administration:

Maintenance: Atezolizumab→Tiragolumab

Table 10 lists the suggested infusion times for treatment administration for carboplatin or cisplatin and etoposide during the induction phase.

Cohort D: Capecitabine Plus Atezolizumab Plus Tiragolumab

On Day 1 of each 21-day cycle, all eligible patients are administered study drugs in the following order:

Capecitabine→atezolizumab→tiragolumab

Table 34 lists the suggested length, dose route, and regimen for treatment administration for capecitabine.

TABLE 34
Treatment Regimen for Cohort with Capecitabine
and Tiragolumab and Atezolizumab

| Cycle Length | Dose, Route, and Regimen ^{a} |
|---|---|
| 3 weeks | 1. Capecitabine: 1250 mg/m² PO BID on Days 1 to 14, |
| | then a 7-day rest period |
| | 2. Atezolizumab: 1200 mg IV infusion on Day 1 |
| | 3. Tiragolimab: 600 mg IV infusion on Day 1 |

BID = twice a day;
PO = orally.
^{a} Drugs listed in order of administration.

Cisplatin

Guidelines for the administration of cisplatin in the different cohorts are shown in Table 11.

Carboplatin

Guidelines for the administration of carboplatin in the different cohorts are shown in Table 12.

Pemetrexed

Guidelines for the administration of pemetrexed in Cohort A is shown in Table 13.

Paclitaxel

Guidelines for the administration of paclitaxel in Cohort B are shown in Table 14.

Etoposide

Guidelines for the administration of etoposide in Cohort C are shown in Table 15.

Capecitabine

Capecitabine is supplied as 150 mg and 500 mg tablets. Patients receive treatment as outlined in Table 35 until unacceptable toxicity or loss of clinical benefit as determined by the investigator.

TABLE 35
Capecitabine Administration Timing and Guidelines

| Chemotherapy | | | |
|---|---|---|---|
| Expansion | | | |
| Cohort | Timing | Dose and Route | Duration |
| D | Day 1 of Cycle 1: Administer first | 1250 mg/m2 PO BID on | Not |
| | dose of capecitabine at the clinic | Days 1-14, followed by | Applicable |
| | prior to the tiragolumab and | a 7-day rest period | |
| | atezolizumab infusion. | | |

BID = twice a day; PO = orally.

Capecitabine is taken with water and within 30 minutes after a meal. Patients are instructed to return used and unused drug to the study site.

D. Concomitant Therapy

Concomitant therapy includes any medication (e.g., prescription drugs, over-the-counter drugs, herbal or homeopathic remedies, and/or nutritional supplements) used by a patient from 7 days prior to screening to the treatment discontinuation visit.

Permitted Therapy

Patients who experience infusion-associated symptoms in this study (Phase Ia or Phase Ib) may be treated symptomatically with acetaminophen, ibuprofen, diphenhydramine, and/or ranitidine or another H2 receptor antagonist, as per standard practice (for sites outside the United States, equivalent medications may be substituted per local practice). Serious infusion-associated events manifested by dyspnea, hypotension, wheezing, bronchospasm, tachycardia, reduced oxygen saturation, or respiratory distress are managed with supportive therapies as clinically indicated (e.g., supplemental oxygen and β2-adrenergic agonists). Premedication with antihistamines may be administered for Cycles ≥2 in Phase Ia or Phase Ib at the discretion of the treating physician.

Systemic corticosteroids, immunosuppresive medications and TNF-α antagonists may attenuate potential beneficial immunologic effects of treatment with tiragolumab as a single agent or with tiragolumab in combination with anti-PD-L1/PD-1 but may be administered at the discretion of the treating physician in an emergency or after consultation with the Medical Monitor. If feasible, alternatives to corticosteroids, immunosuppressive medications or TNF-α antagonists are considered. Premedication for tiragolumab, atezolizumab, bevacizumab, and pembrolizumab may be administered for Cycles ≥2 at the discretion of the treating physician after consultation with the Medical Monitor. The use of inhaled corticosteroids and mineralocorticoids (e.g., fludrocortisone for patients with orthostatic hypotension or adrenocortical insufficiency) is allowed. Physiologic doses of corticosteroids for adrenal insufficiency are allowed. Megestrol administered as an appetite stimulant is also permitted. Planned use of other medications is discussed with the Medical Monitor.

For patients in the Phase Ib chemotherapy expansion cohorts, anti-emetics and systemic steroids can also be given in the following circumstances:
Appropriate antiemetic prophylaxis is administered. The use of a nonsteroidal antiemetic regimen consisting of 5-HT3 receptor and NK1R antagonists is encouraged if feasible (Hesketh et al. J Clin Oncol 2017; 35:3240-3261).
Pretreatment with dexamethasone (or equivalent) may be given to all patients in Cohort A receiving pemetrexed to reduce the incidence and severity of cutaneous reactions. In clinical trials, dexamethasone 4 mg was given by mouth BID the day before, the day of, and the day after pemetrexed administration.
Pretreatment with dexamethasone (or equivalent) may be given to all patients in Cohort B receiving paclitaxel. In clinical trials, dexamethasone 20 mg was given by mouth 12 and 6 hours before paclitaxel administration.

Planned use of prophylactic antibiotics (e.g., following surgical intervention) is discussed with the Medical Monitor. Study drug treatment is held until the antibiotics course has ended.

Patients who use oral contraceptives, hormone-replacement therapy, prophylactic or therapeutic anticoagulation therapy (such as low molecular weight heparin or warfarin at a stable dose level), or other maintenance therapy for non-malignant indications are to continue their use. Males and females of reproductive potential are to use highly effective means of contraception.

For patients in the chemotherapy expansion cohorts, caution is exercised with the use of the following medications:
Caution is used when administering non-steroidal anti-inflammatory drugs (NSAIDs) in patients with mild to moderate renal insufficiency (creatinine clearance [CrCl] from 45 to 79 mL/min) receiving pemetrexed (Cohort A). NSAIDs with short elimination half-lives (e.g., diclofenac, indomethacin) are avoided for a period of 2 days before, the day of, and 2 days following administration of pemetrexed. In the absence of data regarding potential interaction between pemetrexed and NSAIDs with longer half-lives (e.g., meloxicam, nabumetone), patients taking these NSAIDs are to interrupt dosing for at least 5 days before, the day of, and 2 days following pemetrexed. If concomitant administration of NSAIDs is necessary, patients are monitored closely for toxicity, especially myelosuppression, renal, and gastrointestinal toxicity.
Ototoxic drugs such as aminoglycosides and loop diuretics may potentiate ototoxicity in patients receiving cisplatin or carboplatin. Use of antihistamines with cisplatin is reviewed closely as these may mask ototoxicity symptoms such as dizziness and tinnitus. Levels of lithium as well as anti-convulsants (including phenytoin) are monitored closely in patients receiving cisplatin or carboplatin. INR is monitored closely in patients taking oral anti-coagulants and cisplatin.
Elevated coagulation times, as evidenced by increases above baseline INR, have been reported in patients taking warfarin concomitantly with etoposide (Cohort C). Patients receiving warfarin with etoposide are monitored closely with frequent INR.
Patients receiving concomitant capecitabine (Cohort D) and oral coumarin-derivative anticoagulant therapy are to have their anticoagulant response (INR or prothrombin time) monitored frequently in order to adjust the anticoagulant dose accordingly. Altered coagulation parameters and/or bleeding and death have been reported in patients taking capecitabine concomitantly with coumarin-derivative anticoagulants such as warfarin and phenprocoumon. Additional precautions are needed for patients taking capecitabine (Cohort D) who are taking the CYP2C9 substrates leucovorin and phenytoin.

For patients in the non-chemotherapy expansion cohorts, caution is exercised with the use of the following medications:
Osteonecrosis of the jaw has been reported in patients receiving bevacizumab, mainly in combination with bisphosphonates. Thus, caution must be exercised in using bevacizumab in patients receiving concomitant bisphosphonates.

Prohibited Therapy

Use of the following therapies are prohibited during the study:
Any concomitant therapy intended for the treatment of cancer, whether health authority-approved or experimental, including (but not limited to) the following: chemotherapy, hormonal therapy, immunotherapy, radiotherapy, investigational agents, or herbal therapy
   Radiotherapy may be considered for pain palliation (e.g., treatment of known bony metastases) if patients are otherwise deriving benefit. For patients in a dose-escalation cohort in Phase Ia or Phase Ib, palliative radiotherapy is deferred until completion of the DLT assessment window. Study treatment administration may be suspended during radiotherapy with agreement from the Medical Monitor.
   Patients experiencing a mixed response may undergo local therapy (e.g., surgery, stereotactic radiosurgery, radiotherapy, radiofrequency ablation) for control of three or fewer lesions upon approval by the Medical Monitor.
   Patients who undergo radiotherapy to or resection of a target lesion subsequently have that target lesion become non-evaluable for response determination according to RECIST v1.1.
Live, attenuated vaccines (e.g., FluMist® influenza vaccine) are prohibited at any time during the study, and for 5 months following the last dose of study treatment.
Patients are not allowed to receive immunostimulatory agents, including but not limited to IFN-α, IFN-γ, or IL-2, during the entire study. These agents, in combination with atezolizumab, could potentially increase the risk for autoimmune conditions.
Traditional herbal medicines, because these are typically incompletely characterized and may result in unanticipated drug-drug interactions that can cause or confound assessment of toxicity.

Prohibited therapies specific for patients with HCC that are considered for Cohort NC1:
Current use of full-dose anticoagulants, thrombolytic therapy at therapeutic doses, and anti-platelet therapy are prohibited.
   Local label recommended doses for prophylactic use of anticoagulants or thrombolytic therapies is allowed.
   Low-dose aspirin (<325 mg/day) is permitted. Co-administration of proton pump inhibitors is strongly recommended to reduce potential gastrointestinal damage.
   If a patient experiences a venous thromboembolism event while still receiving study drug treatment, it may still be possible for the patient to remain on study medication despite anticoagulation treatment.
Current use of warfarin or Coumadin-like products (includes for prophylactic use) is prohibited. Prophylactic use of low-dose anticoagulation, unfractionated heparin or LMWH is permitted. The preferred choice for anticoagulation treatment is LMWH as per ASCO guidelines (Lyman G H et al. J Clin Oncol 2015; 33:654-6).

Concomitant chronic use of NSAIDs while receiving study drugs is prohibited, with the exception of chronic low-dose aspirin (<325 mg/day). However, for the symptomatic relief of medical conditions (e.g., headache, fever) intermittent or short term intake of oral NSAIDs is allowed, when co-administered with proton pump inhibitors to reduce potential gastrointestinal damage.

D. Inclusion Criteria

Patients must meet the following criteria for study entry:

General Criteria
Signed Informed Consent Form (ICF)
Age ≥18 years
Able to comply with the study protocol, in the investigator's judgment
Eastern Cooperative Oncology Group (ECOG) performance status of 0 or 1
Life expectancy ≥12 weeks
Adequate hematologic and end organ function, defined by the following laboratory results obtained within 14 days prior to the first study treatment (Cycle 1, Day 1):
   Absolute neutrophil count (ANC) ≥1,500 cells/μL
   White blood cell (WBC) count ≥2,500/μL
   Lymphocyte count ≥500/μL
Platelet count ≥100,000/μL (without transfusion within 14 days prior to Cycle 1, Day 1)
   Hemoglobin ≥9 g/dL
   Patients may be transfused or may receive erythropoietic treatment as per local standard of care.
   Total bilirubin ≤1.5×the upper limit of normal (ULN)
   Patients receiving paclitaxel (Cohort B) must have total bilirubin ≤1.25×ULN
   AST and ALT ≤3×ULN
   Alkaline phosphatase 2.5×ULN with the following exception:
   Patients with documented liver or bone metastases may have alkaline phosphatase ≤5×ULN
   Serum albumin ≥2.5 g/dL
   PT and activated partial thromboplastin time (aPTT) ≤1.5×ULN
      This applies only to patients who are not receiving therapeutic anticoagulation.
      Patients receiving therapeutic anticoagulation are on a stable dose.
Measured or calculated creatinine clearance ≥50 mL/min on the basis of the Cockcroft-Gault glomerular filtration rate estimation:

(140−age)×(weight in kilograms)×(0.85 if female)

72×(serum creatinine in mg/dL)
Patients receiving cisplatin (Cohort A or Cohort C) must have measured or calculated creatinine clearance ≥60 mL/min on the basis of the Cockcroft-Gault GFR estimation
Serum pregnancy test for women of childbearing potential (including women who have had a tubal ligation) must be performed and documented as negative within 14 days prior to Cycle 1, Day 1.
For women of childbearing potential: agreement to remain abstinent (refrain from heterosexual intercourse) or use contraception, and agreement to refrain from donating eggs, as defined below:
   Women must remain abstinent or use contraceptive methods with a failure rate of <1% per year during the treatment period and for 90 days after the final dose of tiragolumab, 5 months after the final dose of atezolizumab, 4 months after the final dose of pembrolizumab, and 6 months after the final dose of bevacizumab and chemotherapies (pemetrexed, cisplatin, carboplatin, paclitaxel, etoposide, and capecitabine). Women must refrain from donating eggs during this same period.
   A woman is considered to be of childbearing potential if she is postmenarcheal, has not reached a postmenopausal state (≥12 continuous months of amenorrhea with no identified cause other than menopause), and has not undergone surgical sterilization (removal of ovaries, fallopian tubes, and/or uterus) or another cause as determined by the investigator (e.g., Müllerian agenesis). The definition of childbearing potential may be adapted for alignment with local guidelines or regulations.
   Examples of contraceptive methods with a failure rate of <1% per year include bilateral tubal ligation, male sterilization, hormonal contraceptives that inhibit ovulation, hormone-releasing intrauterine devices, and copper intrauterine devices.
   Hormonal contraceptive methods must be supplemented by a barrier method.
   The reliability of sexual abstinence is evaluated in relation to the duration of the clinical trial and the preferred and usual lifestyle of the patient. Periodic abstinence (e.g., calendar, ovulation, symptothermal, or postovulation methods) and withdrawal are not acceptable methods of contraception.
For men: agreement to remain abstinent (refrain from heterosexual intercourse) or use contraceptive measures, and agreement to refrain from donating sperm, as defined below:
   With a female partner of childbearing potential who is not pregnant, men who are not surgically sterile must remain abstinent or use a condom plus an additional contraceptive method that together result in a failure rate of <1% per year during the treatment period and for 90 days after the last dose of tiragolumab, 3 months after the last dose of capecitabine, and 6 months after the last dose of any other chemotherapy drug. Men must refrain from donating sperm during this same period
   With a pregnant female partner, men must remain abstinent or use a condom during the treatment period and for 90 days after the final dose of tiragolumab, 3 months after the final dose of capecitabine, and 6 months after the final dose of chemotherapies to avoid exposing the embryo.
The reliability of sexual abstinence is evaluated in relation to the duration of the clinical trial and the preferred and usual lifestyle of the patient. Periodic abstinence (e.g., calendar, ovulation, symptothermal, or postovulation methods) and withdrawal are not acceptable methods of contraception.

E. Cancer-Specific Inclusion Criteria
Patients with histologic documentation of locally advanced, recurrent, or metastatic incurable malignancy that has progressed after at least one available standard therapy; or for whom standard therapy has proven to be ineffective or intolerable, or is considered inappropriate; or for whom a clinical trial of an investigational agent is a recognized standard of care.
   If a patient that has progressed after at least one available standard therapy has additional approved standard treatment options available, the study doctor must discuss the risks and benefits of those treatments before informed consent to participate in this study is obtained. This discussion must be documented in patient records.
   For the expansion cohorts of the Phase Ib study only, patients with histologic documentation of locally advanced, recurrent, or metastatic incurable malignancy for whom a clinical trial of an investigational agent in combination with an anti-PD-L1 antibody with and without chemotherapy is considered an acceptable treatment option may also be eligible.
Patients with confirmed availability of representative tumor specimens in formalin-fixed, paraffin-embedded (FFPE) blocks (preferred) or ≥15 unstained slides, with an associated pathology report.
   Acceptable samples may also include core needle biopsies for deep tumor tissue (minimum of three cores) or excisional, incisional, punch, or forceps biopsies for cutaneous, subcutaneous, or mucosal lesions.
   Fine-needle aspiration (FNA) samples, brushings, cell pellets from effusions or ascites, and lavage samples are not acceptable.
   Tumor tissue from bone metastases is not evaluable for PD-L1 and/or TIGIT (T-cell Immunoreceptor with Ig and ITIM domains) expression and is therefore not acceptable.
   If adequate tissue from distinct time points (such as time of initial diagnosis and time of disease recurrence) and/or multiple metastatic tumors is available, priority is given to the tissue most recently collected (ideally subsequent to the most recent systemic therapy). Multiple samples may be collected for a given patient, on the basis of availability; however, the requirement for a block or ≥15 unstained slides is to be satisfied by a single biopsy or resection specimen.
   Prior to signing the main study informed consent form (ICF), patients may sign a pre-screening ICF to specifically allow the collection and testing of archival or fresh tumor specimens.
A patient with insufficient or unavailable archival tissue may be eligible, upon discussion with the Medical Monitor, if the patient meets any of the following:
   Can provide at least 10 unstained, serial slides
   Is willing to consent to and undergo a pretreatment core, punch, or excisional/incisional biopsy sample collection of the tumor (refer to above for acceptable samples)
   Is to be enrolled in a dose-escalation cohort
   Is to be enrolled in the chemotherapy expansion cohorts (Phase Ib)
   Is to be enrolled in the Q4W dosing expansion cohort (Phase Ib)
   For patients in backfill cohorts or expansion cohorts, submitted archival tumor tissue must be evaluated for PD-L1 and/or TIGIT expression prior to enrollment.
Patients with measurable disease per RECIST v1.1
   Previously irradiated lesions are be counted as target lesions.
   Lesions that are intended to be biopsied are not counted as target lesions.

F. Additional Inclusion Criteria for Patients Who Backfill Cleared Cohorts of Phase Ia and Phase Ib
Backfill cohort enrollment is limited to patients whose tumors have PD-L1 and/or TIGIT expression. Therefore, archival tumor tissue must be submitted and evaluated for PD-L1 and/or TIGIT expression prior to enrollment. Patients whose tumor tissue is not evaluable for PD-L1 and/or TIGIT expression are not eligible. PD-L1 and/or TIGIT expression may be evaluated in either immune infiltrating cells or tumor cells. If multiple tumor specimens are submitted (e.g., an archival specimen and tissue from relapsed disease), patients may be eligible if at least one specimen is evaluable for PD-L1 and/or TIGIT. The PD-L1 and/or TIGIT score of the patient is the maximum PD-L1 and/or TIGIT score among the samples, respectively.
Patients must have PD-L1-selected and/or TIGIT-selected tumors of one of the following tumor types: non-small cell lung cancer (NSCLC), renal cell carcinoma (RCC), triple-negative breast cancer (TNBC), melanoma, head and neck squamous cell carcinoma (HNSCC), ovarian cancer (OC), gastric cancer (GC) including gastroesophageal junction (GEJ) cancer, urothelial bladder cancer (UBC), and colorectal cancer (CRC) including microsatellite stable (MSS) and microsatellite instability-low (MSI-Low).
Patients must have accessible lesion(s) that permit a total of one to two biopsies (pretreatment and on-treatment) or one biopsy (on-treatment, if archival tissue can be submitted in place of a pre-treatment biopsy) without unacceptable risk of a significant procedural complication.
   Acceptable samples include core needle biopsies for deep tumor tissue or lymph nodes, or excisional, incisional, punch, or forceps biopsies for cutaneous, subcutaneous, or mucosal lesions. FNAs, cell pellets from effusions or ascites, lavage samples, and bone biopsies are not permitted. Target lesions considered for core needle biopsies are deemed suitable for retrieval of a minimum of three, but ideally five, cores at a given timepoint (minimum diameter 18-gauge).
   If multiple lesions are available, the same tumor lesion is to be biopsied at all timepoints, if feasible, to avoid introduction of heterogeneity related to the site of tumor or metastasis.

G. Additional Inclusion Criteria for Patients in Expansion Cohorts
Enrollment in the expansion cohorts is limited to patients whose tumors are PD-L1 and/or TIGIT selected. Therefore, archival tumor tissue must be submitted and evaluated for PD-L1 and/or TIGIT expression prior to enrollment. Patients whose tumor tissue is not evaluable for PD-L1 and/or TIGIT expression are not eligible. PD-L1 and/or TIGIT expression may be evaluated in either immune infiltrating cells or tumor cells. If multiple tumor specimens are submitted (e.g., an archival specimen and tissue from relapsed disease), patients may be eligible if at least one specimen is evaluable for PD-L1 and/or TIGIT expression. The PD-L1 and/or TIGIT score of the patient is the maximum PD-L1 and/or TIGIT score among the samples, respectively.
Enrollment is managed such that approximately up to half of the accrued patients in each expansion cohort are those who consent to undergo optional biopsies.
For patients enrolled in the serial biopsy expansion cohort in Phase Ib, biopsies are required.

H. Additional Inclusion Criteria for Patients in the Expansion Cohort of Phase Ia
Patients must have PD-L1-selected and/or TIGIT-selected tumors that include the following tumor types: NSCLC, RCC, TNBC, melanoma, HNSCC, OC, GC including GEJ cancer, UBC, and CRC

I. Additional Inclusion Criteria for Patients in Each Indication-Specific Expansion Cohort of Phase Ib
NSCLC Cohorts (CIT-Naive): Patients with histologically confirmed incurable, advanced NSCLC not previously treated with CIT (investigational or approved), including anti-PD-L1/PD-1 and/or anti-CTLA-4 (cytotoxic T-lymphocyte-associated protein 4), for whom a clinical trial of an investigational agent in combination with an anti-PD-L1 antibody is considered an acceptable treatment option, if CIT (including anti-PD-L1/PD-1 agents) is approved as treatment for NSCLC by local regulatory authorities.
   Patients whose tumors have a known sensitizing EGFR mutation must also have experienced disease progression (during or after treatment) or intolerance to treatment with EGFR tyrosine kinase inhibitor(s).
   Patients whose tumors have a known anaplastic lymphoma kinase (ALK) rearrangement must also have experienced disease progression (during or after treatment) or intolerance to treatment with an ALK tyrosine kinase inhibitor(s).
   Patients whose tumors have a known ROS1 rearrangement must also have experienced disease progression (during or after treatment) or intolerance to treatment with an ROS1 tyrosine kinase inhibitor(s).
   Patients whose tumors have a BRAF^{V600E} mutation must also have experienced disease progression (during or after treatment) or intolerance to treatment with dabrafenib in combination with trametinib.
NSCLC Cohort (CIT-Treated): Patients with histologically confirmed incurable, advanced NSCLC previously treated with CIT (investigational or approved) including anti-PD-L1/PD-1
   Patients whose tumors have a known sensitizing EGFR mutation must also have experienced disease progression (during or after treatment) or intolerance to treatment with EGFR tyrosine kinase inhibitor(s).
   Patients whose tumors have a known ALK rearrangement must also have experienced disease progression (during or after treatment) or intolerance to treatment with an ALK tyrosine kinase inhibitor(s).
   Patients whose tumors have a known ROS1 rearrangement must also have experienced disease progression (during or after treatment) or intolerance to treatment with an ROS1 tyrosine kinase inhibitor(s).
   Patients whose tumors have a BRAF^{V600E} mutation must also have experienced disease progression (during or after treatment) or intolerance to treatment with dabrafenib in combination with trametinib.
   Patients must have experienced documented disease progression on CIT monotherapy and/or combination therapy (investigational or approved), which must have included a prior anti-PD-L1/PD-1.
   The prior anti-PD-L1/PD-1 as monotherapy and/or as combination therapy must represent the most recent systemic anti-cancer therapy administered prior to enrollment in this expansion cohort.
      At least approximately 10 patients who experienced a documented best response of investigator-assessed confirmed partial response (PR) or complete response (CR) per RECIST v1.1 at any time while receiving the prior anti-PD-L1/PD-1 as monotherapy or combination therapy may be enrolled.
      At least approximately 10 patients who experienced a documented best response of investigator-assessed stable disease (SD) per RECIST v1.1 at any time while receiving the prior anti-PD-L1/PD-1 as monotherapy and/or as combination therapy may be enrolled.
      At least approximately 10 patients who experienced a documented best response of investigator-assessed progressive disease (PD) per RECIST v1.1 at any time while receiving the prior anti-PD-L1/PD-1 as monotherapy and/or as combination therapy may be enrolled.
      The prior anti-PD-L1/PD-1 as monotherapy and/or as combination therapy must represent the most recent systemic anti-cancer therapy administered prior to enrollment in this expansion cohort.
      Patients who discontinued the prior anti-PD-L1/PD-1 monotherapy and/or combination therapy primarily for toxicity or intolerability are not eligible for enrollment in this expansion cohort.
TNBC Cohort: Patients with histologically confirmed incurable, advanced estrogen receptor (ER)-negative, progesterone receptor-negative, and human epidermal growth factor receptor 2 (HER2)-negative adenocarcinoma of the breast (triple-negative)
   Triple negative status must be documented as defined by the American Society of Clinical Oncology-College of American Pathologists (ASCO-CAP) guidelines:
      <1% of tumor cell nuclei are immunoreactive for ER and <1% of tumor cell nuclei are immunoreactive for progesterone receptor
   and
   HER2 tests demonstrate immunohistochemistry (IHC) 1+, IHC 0, or in situ hybridization (ISH) negative
CRC Cohort: Patients with histologically confirmed incurable, advanced adenocarcinoma of the colon or rectum
   Patients with tumors of appendiceal origin are not eligible.
GC Cohort: Patients with histologically confirmed inoperable, locally advanced or metastatic or recurrent gastric or GEJ adenocarcinoma, not amenable to curative therapy
   Patients with Type 1 GEJ tumor, defined as adenocarcinoma of the distal esophagus with the tumor center located within 1 to 5 cm above the anatomic esophagogastric junction, are eligible for the study
      Patients with esophageal cancers (squamous cell carcinoma or adenocarcinoma) may be eligible following a discussion with the Medical Monitor.
   Patients whose tumors are HER2-positive must also have experienced disease progression (during or after treatment) or intolerance to treatment with HER2-targeting antibody/HER2 inhibitor(s).
      HER2-positivity is defined as either IHC 3+ or IHC 2+/ISH+(where ISH positivity is defined as a HER2:CEP17 ratio of ≥2), as assessed by a local laboratory test on the primary tumor or on a metastatic lesion
      Patients who have not had HER2 testing due to insufficient or unavailable tissue (e.g., archival and/or biopsy), and thus the HER2 status of the tumor is unknown, may still be eligible following a discussion with the Medical Monitor.
HNSCC Cohort: Patients with histologically confirmed inoperable, locally advanced or metastatic, recurrent, or persistent head and neck squamous cell carcinoma (oral cavity, oropharynx, hypopharnyx, or larynx), not amenable to curative therapy
   Patients with HNSCC of any other primary anatomic location in the head and neck, patients with HNSCC of unknown primary, or patients with tumors of non-squamous histologies are not eligible.
   Patients with HNSCC of the nasopharynx may be eligible following a discussion with the Medical Monitor.
   HPV status for the HNSCC must be known.
UBC Cohort: Patients with histologically confirmed incurable advanced transitional cell carcinoma of the urothelium (including renal pelvis, ureters, urinary bladder, and urethra)
   Patients with mixed histologies are required to have a dominant transitional cell pattern.
Melanoma Cohort: Patients with histologically confirmed incurable, advanced metastatic melanoma
   Patients with melanoma for whom a clinical trial of an investigational agent in combination with an anti-PD-L1/PD-1 antibody is considered an acceptable treatment option, if CIT (including anti-PD-L1/PD-1 agents and/or anti-CTLA-4 agents) is approved as treatment for melanoma by local regulatory authorities.
   Patients whose tumors have a known BRAFV600 mutation must also have experienced disease progression (during or after treatment) or intolerance with BRAF inhibitor (s) and/or mitogen-activated protein kinase (MEK) inhibitor(s).
   Enrollment is managed so that no more than approximately 20% of patients in this cohort are patients with ocular (uveal) melanoma.
OC Cohort: Patients with histologically confirmed incurable, advanced epithelial ovarian, fallopian tube, or primary peritoneal cancer
   Borderline ovarian epithelial neoplasms (e.g., tumors of low malignant potential, atypical proliferative tumors) are excluded.
RCC Cohort: Patients with histologically confirmed incurable, advanced RCC with component of clear cell histology and/or component of sarcomatoid histology
   Patients with RCC for whom a clinical trial of an investigational agent in combination with an anti-PD-L1 antibody is considered an acceptable treatment option, if CIT (including anti-PD-L1/PD-1 agents) is approved as treatment for RCC by local regulatory authorities.

J. Additional Inclusion Criteria for Patients in the Serial Biopsy Expansion Cohort of Phase Ib
Patients must have PD-L1-selected and/or TIGIT-selected tumors that may include the following tumor types: melanoma, OC, RCC, and UBC
   Patients with melanoma or RCC for whom a clinical trial of an investigational agent in combination with an anti-PD-L1 antibody is considered an acceptable treatment option, if CIT (including anti-PD-L1/PD-1 agents) is approved as treatment for these indication(s) by local regulatory authorities.
Patients must have accessible lesion(s) that permit a total of one to two biopsies (pretreatment and on-treatment) or one biopsy (on-treatment, if archival tissue can be submitted in place of a pre-treatment biopsy) without unacceptable risk of a significant procedural complication.
   Acceptable samples include core needle biopsies for deep tumor tissue or lymph nodes, or excisional, incisional, punch, or forceps biopsies for cutaneous, subcutaneous, or mucosal lesions. FNA, cell pellets from effusions or ascites, lavage samples, and bone biopsies are not permitted. Target lesions considered for core needle biopsies are deemed suitable for retrieval of a minimum of three, but ideally five, cores at a given timepoint (minimum diameter 18-gauge).
   If multiple lesions are available, the same tumor lesion is to be biopsied at all timepoints, if feasible, to avoid introduction of heterogeneity related to the site of tumor or metastasis.

K. Exclusion Criteria

Patients who meet any of the following criteria are excluded:

General Exclusion Criteria
Inability to comply with study and follow-up procedures
Pregnancy, lactation, or breastfeeding
Significant cardiovascular disease, such as New York Heart Association cardiac disease (Class II or greater), myocardial infarction within the previous 3 months, unstable arrhythmias, and/or unstable angina
Known clinically significant liver disease, including active viral, alcoholic, or other hepatitis, cirrhosis, and inherited liver disease or current alcohol abuse
Poorly controlled Type 2 diabetes mellitus defined as a screening hemoglobin A1c≥8% or a fasting plasma glucose ≥160 mg/dL (or 8.8 mmol/L)
Major surgical procedure within 28 days prior to Cycle 1, Day 1, or anticipation of need for a major surgical procedure during the course of the study
Any other diseases, metabolic dysfunction, physical examination finding, and/or clinical laboratory finding giving reasonable suspicion of a disease or condition that contraindicates the use of an investigational drug or that may affect the interpretation of the results or may render the patient at high risk from treatment complications.

L. Cancer-Specific Exclusion Criteria
Any anti-cancer therapy, whether investigational or approved, including chemotherapy, hormonal therapy, and/or radiotherapy, within 3 weeks prior to initiation of study treatment, with the following exceptions:
   Hormonal therapy with gonadotropin-releasing hormone (GnRH) agonists or antagonists for prostate cancer
   Hormone-replacement therapy or oral contraceptives
   Tyrosine kinase inhibitor(s) (TKIs) approved by local regulatory authorities for treatment of cancer that have been discontinued >7 days prior to Cycle 1, Day 1; baseline scans must be obtained after discontinuation of prior TKIs, and criteria pertaining to adverse events attributed to prior cancer therapies must be met
   Herbal therapy >1 week before Cycle 1, Day 1
   Palliative radiotherapy for painful metastases or metastases in potentially sensitive locations (e.g., epidural space) >2 weeks prior to Cycle 1, Day 1
   Patients who crossover from the Phase Ia portion of this study to the Phase Ib study with tiragolumab and atezolizumab are allowed to have been treated with tiragolumab as a single agent prior to entering the Phase Ib study.
Eligibility based on prior treatment with CIT depends on the mechanistic class of the drug and the cohort for which the patient is being considered, as described below. In addition, all criteria pertaining to adverse events attributed to prior cancer therapies must be met.

All Cohorts (Dose-Escalation, Backfill, and Expansion) in Phase Ia and Phase Ib:
Prior anti-TIGIT agents are not allowed, with the following exception:
Patients treated with tiragolumab in the Phase Ia study may be allowed to enroll in the Phase Ib study, provided that the criteria specified are met.
Prior treatment with cancer vaccines and/or cytokines is allowed provided that at least 6 weeks or 5 half-lives of the drug, whichever is shorter, have elapsed between the last dose and the proposed Cycle 1, Day 1.
Minimum washout is 3 weeks for any prior systemic cancer therapy.
Prior cancer immunotherapy not explicitly described in this protocol is to be discussed with the Medical Monitor to determine potential eligibility.

Dose-Escalation and Backfill Cohorts (Enrolling During Dose-Escalation) in Phase Ia and Phase Ib:
Prior treatment with immune checkpoint inhibitors (such as anti-PD-L1/PD-1), immunomodulatory MAbs, and/or MAb-derived therapies is allowed provided that at least 5 elimination half-lives of the drug, have elapsed between the last dose and the proposed Cycle 1, Day 1.
Prior treatment with immunomodulators, including toll-like receptor (TLR) agonists, inhibitors of indoleamine 2,3-dioxygenase or tryptophan-2,3-dioxygenase (IDO/TDO), or agonists of OX40 is allowed provided that at least 6 weeks have elapsed between the last dose and the proposed Cycle 1, Day 1.

Dose-Expansion and Backfill Cohorts (Enrolling During Dose-Expansion) in Phase Ia and Phase Ib:
Prior treatment with immune checkpoint inhibitors, immunomodulatory MAbs, and/or MAb-derived therapies is allowed provided that at least 6 weeks have elapsed between the last dose and the proposed Cycle 1, Day 1, with the following exceptions:
   Prior anti-PD-L1/PD-1 is subject to at least a 3-week washout period
   Prior anti-CTLA-4 is subject to a 6-week washout period
In the NSCLC CIT-Treated expansion cohort in Phase Ib, the most recent systemic treatment is to have been anti-PD-L1/PD-1 as monotherapy or in combination.
In the NSCLC CIT-Naive expansion cohort in Phase Ib, prior treatment with immune checkpoint inhibitors (such as anti-PD-L1/PD-1), immunomodulatory MAbs, and/or MAb-derived therapies is not allowed.
   Prior treatment with immunomodulators, including TLR agonists, inhibitors of IDO/TDO, or agonists of OX40, is allowed provided that at least 5 half-lives of the drug or a minimum of 3 weeks, have elapsed between the last dose of the prior treatment and the proposed Cycle 1, Day 1, with the following exception:
   In the NSCLC CIT-Naive expansion cohort in Phase Ib, prior treatment with other immunomodulators is not allowed.
Any history of an immune-mediated Grade 4 adverse event attributed to prior cancer immunotherapy (other than endocrinopathy managed with replacement therapy or asymptomatic elevation of serum amylase or lipase)
Any history of an immune-mediated Grade 3 adverse event attributed to prior cancer immunotherapy (other than endocrinopathy managed with replacement therapy or asymptomatic elevation of serum amylase or lipase) that resulted in permanent discontinuation of the prior immunotherapeutic agent and/or occurred ≤6 months prior to Cycle 1 Day 1.
Adverse events from prior anti-cancer therapy that have not resolved to Grade ≤1 except for alopecia, vitiligo, or endocrinopathy managed with replacement therapy
All immune-mediated adverse events related to prior cancer immunotherapy (other than endocrinopathy managed with replacement therapy or stable vitiligo) must have resolved completely to baseline.
   Patients treated with corticosteroids for immune-mediated adverse events must demonstrate absence of related symptoms or signs for >4 weeks following discontinuation of corticosteroids.
Patients with the pulmonary lymphoepithelioma-like carcinoma subtype of NSCLC
Primary CNS malignancy, untreated CNS metastases, or active CNS metastases (progressing or requiring corticosteroids for symptomatic control)
   Patients with a history of treated CNS metastases are eligible, provided they meet all of the following criteria:
   Measurable disease outside the CNS
   No ongoing requirement for corticosteroids as therapy for CNS metastases, with corticosteroids discontinued for ≥2 weeks prior to enrollment with no ongoing symptoms attributable to CNS metastases
   Anticonvulsants at a stable dose are allowed.
   Radiographic demonstration of improvement upon the completion of CNS-directed therapy and no evidence of interim progression between the completion of CNS-directed therapy and the screening radiographic study
   The screening CNS radiographic study is ≥4 weeks since completion of radiotherapy
Leptomeningeal disease
Uncontrolled tumor-related pain
   Symptomatic lesions amenable to palliative radiotherapy (e.g., bone metastases or metastases causing nerve impingement) are treated prior to enrollment.
   Asymptomatic metastatic lesions whose further growth would likely cause functional deficits or intractable pain (e.g., epidural metastasis that is not currently associated with spinal cord compression) are considered for loco-regional therapy if appropriate prior to enrollment.
Uncontrolled pleural effusion, pericardial effusion, or ascites requiring recurrent drainage procedures (once monthly or more frequently)
   Patients with indwelling catheters (e.g., PleurX catheters) are allowed.
Malignancies other than disease under study within 5 years prior to Cycle 1, Day 1, with the exception of those with a negligible risk of metastasis or death (such as adequately treated carcinoma in situ of the cervix, basal or squamous cell skin cancer, localized prostate cancer, or ductal carcinoma in situ)
Uncontrolled hypercalcemia (>1.5 mmol/L ionized calcium or Ca>12 mg/dL or corrected serum calcium ≥ULN) or symptomatic hypercalcemia requiring continued use of bisphosphonate therapy or denosumab.
   Patients who are receiving bisphosphonate therapy or denosumab specifically to prevent skeletal events and who do not have a history of clinically significant hypercalcemia are eligible. However, patients on denosumab must be willing and eligible to receive a bisphosphonate instead of denosumab while on study.
Patient has spinal cord compression not definitively treated with surgery and/or radiation or previously diagnosed and treated spinal cord compression without evidence that disease has been clinically stable for ≥2 weeks prior to screening.

M. Treatment-Specific Exclusion Criteria
History of autoimmune disease, including but not limited to systemic lupus erythematosus, rheumatoid arthritis, inflammatory bowel disease, vascular thrombosis associated with antiphospholipid syndrome, Wegeners granulomatosis, Sjögren's syndrome, Bell's palsy (of autoimmune etiology only), Guillain-Barré syndrome, multiple sclerosis, vasculitis, or glomerulonephritis, with the following caveats:
   Patients with a history of autoimmune hypothyroidism on a stable dose of thyroid replacement hormone may be eligible.
   Patients with eczema, psoriasis, lichen simplex chronicus, or vitiligo with dermatologic manifestations only (e.g., no psoriatic arthritis) may be eligible provided that they meet the following conditions:
      Rash must cover less than 10% of the body surface area.
      Disease is well controlled at baseline and only requires low potency topical steroids.
      No acute exacerbations of underlying condition within the last 12 months (e.g., not requiring psoralen plus ultraviolet A radiation (PUVA), methotrexate, retinoids, biologic agents, oral calcineurin inhibitors, high potency or oral steroids)
Treatment with systemic immunosuppressive medications (including but not limited to prednisone >10 mg/day, cyclophosphamide, azathioprine, methotrexate, thalidomide, and tumor necrosis factor-α [TNF-α] antagonists) within 2 weeks prior to Cycle 1, Day 1
   Patients who have received acute, low-dose, systemic immunosuppressant medications (e.g., a one-time dose of dexamethasone for nausea) may be enrolled in the study after discussion with and approval by the Medical Monitor.
   The use of inhaled corticosteroids (e.g., fluticasone for chronic obstructive pulmonary disease) is allowed.
   The use of oral mineralocorticoids (e.g., fludrocortisone for patients with orthostatic hypotension) is allowed.
   Physiologic doses of corticosteroids for adrenal insufficiency are allowed.
History of idiopathic pulmonary fibrosis, pneumonitis (including drug-induced), organizing pneumonia (i.e., bronchiolitis obliterans, cryptogenic organizing pneumonia, etc.), or evidence of active pneumonitis on screening chest CT scan
   History of radiation pneumonitis in the radiation field (fibrosis) is permitted.
Positive test for HIV infection
Active hepatitis B (defined as having a positive hepatitis B surface antigen [HBsAg] test at screening)
   Patients with past or resolved hepatitis B infection (defined as having a negative HBsAg test and a positive IgG antibody to hepatitis B core antigen [anti-HBc]) are eligible.
   Hepatitis B virus (HBV) DNA must be obtained in these patients prior to Cycle 1, Day 1, and must demonstrate no active infection.
Active hepatitis C
   Patients positive for hepatitis C virus (HCV) antibody are eligible only if polymerase chain reaction (PCR) is negative for HCV RNA.
Active EBV infection and known or suspected chronic active EBV infection at screening
   Patients positive for EBV IgG and/or EBNA are eligible only if EBV IgM and/or EBV PCR are negative.
Active tuberculosis
Severe infections within 4 weeks prior to Cycle 1, Day 1, including but not limited to hospitalization for complications of infection, bacteremia, or severe pneumonia
Recent infections not meeting the above criteria for severe infections, including the following:
   Signs or symptoms of infection within 2 weeks prior to Cycle 1, Day 1
   Received oral or IV antibiotics within 2 weeks prior to Cycle 1, Day 1
   Patients receiving prophylactic antibiotics (e.g., for prevention of a urinary tract infection or chronic obstructive pulmonary disease) are eligible.
Prior allogeneic bone marrow transplantation or prior solid organ transplantation
Administration of a live, attenuated vaccine within 4 weeks before Cycle 1, Day 1 or anticipation that such a live attenuated vaccine is required during the study
   Influenza vaccination is to be given during influenza season only. Patients must not receive live, attenuated influenza vaccine (e.g., FluMist®) within 4 weeks prior to Cycle 1, Day 1 or at any time during the study, and for 5 months following the last study treatment.
History of severe allergic, anaphylactic, or other hypersensitivity reactions to chimeric or humanized antibodies or fusion proteins
Known hypersensitivity to CHO-cell products
Allergy or hypersensitivity to components of the atezolizumab formulation

N. Exclusion Criteria Specific to Chemotherapy Expansion Cohorts
Known severe allergy or hypersensitivity to platinum or platinum-containing compounds
Exclusion Criteria Specific to Cisplatin
Patients with known hearing impairment (cisplatin treatment only)
Grade ≥2 peripheral neuropathy as defined by NCI CTCAE v4.0 criteria (cisplatin treatment only)
Measured or calculated creatinine clearance ≤60 mL/min (cisplatin treatment only) on the basis of the Cockcroft-Gault GFR estimation:

$$\frac{( 140 - age ) \times ( weight in kilograms ) \times ( 0.85 if female )}{72 \times ( serum creatinine in mg \text{/} dL )}$$

O. Exclusion Criteria Specific to Cohort A
Known severe allergy or hypersensitivity to pemetrexed

P. Exclusion Criteria Specific to Cohort B
Known severe allergy or hypersensitivity to paclitaxel
Known history of severe hypersensitivity reactions to products containing Cremophor® EL (e.g., cyclosporin for injection concentrate and teniposide for injection concentrate)

Q. Exclusion Criteria Specific to Cohort C
Known severe allergy or hypersensitivity to etoposide

R. Exclusion Criteria Specific to Cohort D
Hypersensitivity to any component of capecitabine drug formulation
Inability to swallow pills
Malabsorption syndrome, disease significantly affecting gastrointestinal function, resection of the stomach or small bowel, or ulcerative colitis
Known dihydropyrimidine dehydrogenase deficiency or history of severe and unexpected reactions to fluoropyrimidine therapy
Requirement for concurrent use of the antiviral agent sorivudine (antiviral) or chemically related analogs, such as brivudine (use of these drugs is not allowed within 4 weeks of initiation of study treatment that includes capecitabine)

S. Exclusion Criteria Specific to Cohort NC1
Inadequately controlled hypertension (defined as systolic blood pressure >150 mmHg and/or diastolic blood pressure >100 mmHg), based on an average of ≥3 blood pressure readings on ≥2 sessions; anti-hypertensive therapy to achieve these parameters is allowed
History of hypertensive crisis or hypertensive encephalopathy
Significant vascular disease (e.g., aortic aneurysm requiring surgical repair or recent peripheral arterial thrombosis) within 6 months prior to initiation of study treatment
History of hemoptysis (≥2.5 mL of bright red blood per episode) within 1 month prior to initiation of study treatment
Evidence of bleeding diathesis or significant coagulopathy (in the absence of therapeutic anticoagulation)
Current or recent (≤10 days prior to initiation of study treatment) use of aspirin (>325 mg/day) or clopidogrel (>75 mg/day)
Note: The use of full-dose oral or parenteral anticoagulants for therapeutic purpose is permitted as long as the INR and/or aPTT is within therapeutic limits (according to institution standards) within 7 days prior to initiation of study treatment and the patient has been on a stable dose of anticoagulants for ≥2 weeks prior to initiation of study treatment; prophylactic use of anticoagulants is allowed
Core biopsy or other minor surgical procedure, excluding placement of a vascular access device, within 3 days prior to initiation of study treatment
History of abdominal or tracheoesophageal fistula, gastrointestinal perforation, or intra-abdominal abscess within 6 months prior to initiation of study treatment
History of intestinal obstruction and/or clinical signs or symptoms of gastrointestinal obstruction, including subocclusive or occlusive syndrome related to the underlying disease, within 6 months prior to initiation of study treatment or requirement for routine parenteral hydration, parenteral nutrition, or tube feeding within 6 months prior to initiation of study treatment
Patients with signs or symptoms of subocclusive or occlusive syndrome or with intestinal obstruction at the time of initial diagnosis may be enrolled if they had received definitive (surgical) treatment for symptom resolution
Evidence of abdominal free air that is not explained by paracentesis or recent surgical procedure
Serious, non-healing or dehiscing wound, active ulcer, or untreated bone fracture
Grade ≥2 proteinuria, as demonstrated by ≥2+protein on dipstick urinalysis and ≥1.0 g of protein in a 24-hour urine collection
   All patients with ≥2+ protein on dipstick urinalysis at screening must undergo a 24-hour urine collection for protein.
   Patients with <2+ protein on dipstick urinalysis are eligible for the study.
Metastatic disease that involves major airways or blood vessels, or centrally located mediastinal tumor masses (<30 mm from the carina) of large volume
History of intra-abdominal inflammatory process within 6 months prior to initiation of study treatment, including, but not limited to, peptic ulcer disease, diverticulitis, or colitis
Radiotherapy within 28 days or abdominal/pelvic radiotherapy within 60 days prior to initiation of study treatment
Major surgical procedure, open biopsy, or significant traumatic injury within 28 days prior to initiation of study treatment; or abdominal surgery, abdominal interventions or significant abdominal traumatic injury within 60 days prior to initiation of study treatment; or anticipation of need for major surgical procedure during the course of the study or non-recovery from side effects of any such procedure

T. Assessments

Patients are closely monitored for safety and tolerability and are assessed for toxicity prior to each dose of tiragolumab as a single agent (Phase Ia) or tiragolumab in combination with the following: atezolizumab, atezolizumab with chemotherapy, atezolizumab with bevacizumab, or pembrolizumab (Phase Ib). Dosing occurs only if the clinical assessment and local laboratory test results are acceptable.

All assessments are performed on the day of the scheduled visit date unless a time window is specified. Assessments scheduled on the days of study treatment are performed before the infusion of study drug(s) unless otherwise noted.

Medical History, Baseline Conditions, Concomitant Medication, and Demographic Data

Medical history includes cancer history (including but not limited to prior cancer therapies and/or prior CITs and procedures and tumor characteristics such as hormone receptor status or mutation status), other clinically significant diseases, surgeries, smoking history, use of alcohol and/or drugs of abuse, reproductive status and all medications (e.g., prescription drugs, over-the-counter drugs, herbal or homeopathic remedies, and nutritional supplements) used by the patient within 7 days prior to the screening visit.

For those patients who crossover from the Phase Ia to the Phase Ib portion of the study, the re-screening medical history at the time of treatment re-initiation upon disease progression includes any cancer-related procedures or any adverse events related to study treatment occurring after initial treatment discontinuation that were not captured during the protocol-specified follow-up.

Demographic data include age, sex, and self-reported race/ethnicity. Race/ethnicity is recorded because of the potential contribution of this variable to differences in observed PK, pharmacodynamics, toxicity, and/or response to treatment.

Vital Signs

Vital signs include measurements of temperature, respiratory rate, pulse rate, and systolic and diastolic blood pressures while the patient is in a seated position.

On study treatment days, vital signs are measured within 60 minutes before the first study drug infusion of the day.

For the first infusion of tiragolumab in Phase Ia, measure vital signs every 15 (±5) minutes during the infusion, at the end of the infusion (±5 minutes), and 30 (±10) minutes, 120 (±15) minutes, and 240 (±15) minutes after the end of infusion.

For the first infusion of tiragolumab in Phase Ib, measure vital signs every 15 (±5) minutes during the infusion, at the end of the infusion (±5 minutes), and 30 (±10) minutes after the end of the infusion. For all Phase Ib cohorts without chemotherapy, the first infusion of atezolizumab or pembrolizumab is administered 60 minutes after completion of the tiragolumab infusion; the first infusion of bevacizumab is administered 60 minutes after completion of the atezolizumab infusion.

For Phase Ib cohorts with chemotherapy, the first infusion of tiragolumab is administered 60 minutes after completion of the atezolizumab infusion.

For the first and subsequent infusions of atezolizumab and pembrolizumab, and subsequent infusions of tiragolumab, vital signs are measured during the infusion and 30 (±10) minutes after the infusion if clinically indicated or if symptoms occurred in the prior infusion.

For the first infusion of bevacizumab, vital signs are measured during the infusion, at the end of the infusion (±5 minutes), and 30 (±10) minutes after the infusion if clinically indicated or if symptoms occurred in the prior infusion.

The interval in subsequent infusions between tiragolumab and atezolizumab or pembrolizumab, or between atezolizumab and bevacizumab (for all Phase Ib cohorts without chemotherapy), or between atezolizumab and tiragolumab (for Phase Ib cohorts with chemotherapy) is 30 minutes if the previous tiragolumab or atezolizumab infusion was tolerated without an IRR or 60 minutes if the patient experienced an IRR with the previous tiragolumab or atezolizumab infusion.

In the chemotherapy expansion A, B, and C cohorts, additional vital signs are measured and recorded on Cycle 1 within 30 (±10) minutes after completion of the last chemotherapy infusion (e.g., Cohort A: after carboplatin or cisplatin, Cohort B: after carboplatin, Cohort C: after etoposide). At subsequent cycles, additional vital signs are measured and recorded after the chemotherapy infusion if clinically indicated.

Vital signs collected at the screening visit are recorded. For each visit thereafter, only those vital signs that are obtained prior to the first study drug infusion of the day or that constitute an adverse event (e.g., temperature for event of fever) or a primary manifestation of an adverse event (e.g., blood pressure associated with an infusion-related reaction or heart rate associated with an arrhythmia) are recorded. All vital signs collected per protocol are documented in the patient's medical record.

Blood oxygen saturation is measured at baseline by pulse oximetry.

Physical Examinations

A complete physical examination performed at screening includes an evaluation of the head, eyes, ears, nose, and throat, and the cardiovascular, dermatological, musculoskeletal, respiratory, gastrointestinal, genitourinary, and neurological systems.

ECOG Performance Status is assessed.

At subsequent visits (or as clinically indicated), limited, symptom-directed physical examinations are performed. Changes from baseline abnormalities are recorded in the patient's medical record. New or worsened clinically significant abnormalities are recorded as adverse events.

As part of tumor assessments, the physical examination also includes evaluation for lymphadenopathy, splenomegaly, hepatomegaly, and cutaneous neoplasms or metastases. All patients are monitored for symptoms of CNS metastases and such reported symptoms are followed by a full neurological examination. A brain magnetic resonance imaging (MRI) scan or contrast-enhanced head CT scan is done as clinically indicated to confirm or refute new or worsening brain involvement.

Tumor and Response Evaluations

Screening

All known sites of disease must be documented at screening and re assessed at each subsequent tumor evaluation.

Screening and subsequent tumor assessments must include CT scans (with IV contrast unless contraindicated and oral contrast as appropriate per institutional standards), or MRI scans, of the chest, abdomen, and pelvis. If a CT scan for tumor assessment is performed in a positron emission tomography (PET)/CT scanner, the CT acquisition must be consistent with the standards for a full-contrast CT scan.

Brain imaging (either MRI or contrast-enhanced CT) is required at screening for patients with treated brain metastases and as clinically indicated based on symptoms or signs suggestive of new or worsening CNS metastases. In the event of an equivocal head CT, a brain MRI is required to clarify the presence or extent of suspected brain metastases.

Further investigations such as bone scans and CT scans of the neck are also performed as indicated by the underlying disease (e.g., a head and neck CT scan is indicated for patients with HNSCC) and if there is any clinical suspicion of disease at any site that may not be demonstrated by the minimum schedule of assessments listed above. At the investigator's discretion, other methods of assessment of measurable disease as per RECIST v1.1 may be used.

The same radiographic procedures used to assess disease sites at screening are used throughout the study (e.g., the same contrast protocol for CT scans). Stable brain metastases must be evaluated with each tumor assessment with the same radiographic procedure as the baseline study. Patients without brain metastases do not need a brain scan for tumor assessment unless clinically warranted. Response is assessed by the investigator on the basis of physical examinations and the imaging modalities detailed above, using RECIST v1.1. The investigator's assessment of overall tumor response at all timepoints are only based on RECIST v1.1. Assessments are performed by the same evaluator if possible to ensure internal consistency across visits.

Tumor assessments are performed. At the investigator's discretion, scans may be performed at any time if PD is suspected.

After initial study treatment discontinuation (if discontinuation were for reasons other than disease progression), follow-up tumor assessments are performed until death, disease progression, initiation of another systemic anti-cancer therapy, loss to follow-up, withdrawal of consent, or study termination, whichever occurs first.

Patients who continue treatment beyond radiographic disease progression per RECIST v1.1 are monitored with a follow-up scan in 6 (±2) weeks (i.e., at the next scheduled tumor assessment when the scan frequency is every 2 cycles or as an unscheduled tumor assessment when the scan frequency is every 4 cycles), or earlier if clinically indicated. Tumor assessments are continued every 2 cycles thereafter until two consecutive scans demonstrate stability or improvement with respect to the first scan that showed radiographic disease progression, at which point the scan frequency reverts or transitions to every 4 cycles if applicable. Investigator assessment of overall tumor response at all timepoints are only based on RECIST 1.1.

Laboratory, Biomarker, and Other Biological Samples

Local Laboratory Tests in Phase Ia and Phase Ib

The following laboratory tests are performed at the study site's local laboratory:
Hematology (CBC, including RBC count, hemoglobin, hematocrit, WBC count with differential [neutrophils, eosinophils, lymphocytes, monocytes, basophils, and other cells], and platelet count)
Serum/plasma chemistries (sodium, potassium, chloride, bicarbonate, BUN or urea, creatinine, glucose, calcium, magnesium, phosphorus, total bilirubin, ALT, AST, alkaline phosphatase, LDH, total protein, and albumin)
Serum ferritin and C-reactive protein (CRP)
Coagulation (PT, aPTT, and INR)
Amylase and lipase
Pregnancy test: All women of childbearing potential (including those who have had a tubal ligation) have a serum pregnancy test at screening. Urine pregnancy tests are performed at regular intervals for the duration of study treatment. If a urine pregnancy test result is positive, dosing is delayed until the patient's status is determined by a serum pregnancy test.
Urinalysis (specific gravity, pH, glucose, protein, ketones, and blood)
Thyroid function testing (thyroid-stimulating hormone [TSH], free T3, and free T4)
EBV serology (EBV IgM, EBV IgG, and/or EBNA]) and/or EBV PCR
   EBV IgM test and/or EBV PCR test is required prior to Cycle 1, Day 1 for consideration of eligibility if the patient has positive serology for EBV IgG and/or EBNA.
CMV serology (CMV IgG)
HBV serology (HBsAg, antibodies against HBsAg, and hepatitis B core antigen)
   HBV DNA test is to be obtained prior to Cycle 1, Day 1 if the patient has positive serology for anti-HBc.
HCV serology (anti-HCV)
   HCV RNA test is required prior to Cycle 1, Day 1 for consideration of eligibility if the patient has positive serology for anti-HCV.
All patients are tested for HIV prior to the inclusion into the study and HIV-positive patients are excluded from the study
Tumor markers (if applicable and informative), including but not limited to CA-125, CA19-9, carcinoembryonic antigen (CEA), prostate-specific antigen (PSA), beta-hCG, AFP, CA15-3, and CA27.29

Central Laboratory Tests in Phase Ia and Phase Ib

The tests listed below are performed at a central laboratory. Samples collected for PK or ADA analysis may be needed for additional immunogenicity characterization, PK, biomarker, and/or immunogenicity assay development and validation; therefore, those samples are destroyed no later than 5 years after the final Clinical Study Report has been completed, or earlier depending on local regulations.

Assessments Performed on Blood Samples
ADA assays
   Serum samples are obtained for measurement of ADAs to tiragolumab using validated assays (Phase Ia and Phase Ib)
   Serum samples are obtained for measurement of ADAs to atezolizumab using validated assays (Phase Ib only)
PK assays
   Serum samples are obtained for measurement of tiragolumab concentration using a validated assay (Phase Ia and Phase Ib)
   Serum samples are obtained for measurement of atezolizumab concentration using a validated assay (Phase Ib only)
   Plasma samples are obtained for measurement of cisplatin, carboplatin, pemetrexed, paclitaxel, capecitabine and etoposide concentrations using validated assays (Phase Ib chemotherapy expansion cohorts only).
Auto-antibody testing
   Serum samples are obtained for auto-antibody testing, including but not necessarily limited to anti-nuclear antibody, anti-double-stranded DNA, anti-neutrophil cytoplasmic antibodies, and thyroid peroxidase antibody, to be performed based on clinical events during the study, either in individual patients or across the study population.
Biomarker assays in blood
   Blood samples are obtained for biomarker evaluation from all eligible patients at the screening visit and on treatment.
Whole genome sequencing (WGS)
   A single blood sample is collected for WGS and may be sent to one or more laboratories for analysis. WGS data and associated clinical data may be shared with researchers who are not participating in the study or may be submitted to government or other health research databases for broad sharing with other researchers to perform research on human health and disease. Study participants are not identified by name or any other personally identifying information.
   WGS is contingent upon the review and approval by each site's IRB/EC and, if applicable, an appropriate regulatory body. If the site has not been granted approval for WGS, this assessment is not applicable at that site.
   WGS data are analyzed in the context of this study and explored in aggregate with other studies to better understand disease pathobiology and guide the development of new therapeutic approaches. Given the complexity and exploratory nature of these analyses, WGS data and analyses are for research purposes only and are not shared with investigators or study participants.

These samples are stored indefinitely, until depleted, or until patient requests that their samples be destroyed.

Assessments Performed on Tumor Samples

The status of immune-related and tumor-related biomarkers (including but not limited to the expression of PD-L1, TIGIT, PVR, and/or CD226 on specific cell types; and the prevalence and/or activation of infiltrating T cells) may be evaluated using methods including, but not limited to, IHC, IF, and qRT-PCR in both archival and fresh tumor samples. Tumor RNA and/or DNA may be purified and subject to characterization by RNA sequencing and WGS. Additional exploratory biomarkers may also be assessed if guided by clinical and nonclinical data.
Archival tumor tissue: For all patients enrolled in the study
   If available, archival tumor tissue samples obtained outside of this study for other purposes are collected from all patients (paraffin blocks are preferred; at least 15 unstained slides are acceptable). For all patients in Phase Ia or Phase Ib, availability of archival tumor tissue must be confirmed prior to study entry. Fine-needle aspirates, cell pellets from effusions or ascites, lavage samples, and bone biopsies do not satisfy the requirement for archival tissue.
   If adequate tissue from distinct timepoints (such as time of initial diagnosis and time of disease recurrence) and/or multiple metastatic tumors is available, priority is given to the tissue most recently collected (ideally subsequent to the most recent systemic therapy).
   A patient with insufficient or unavailable archival tissue may still be eligible.
   Archival tumor tissue is used for evaluation of potential predictive biomarkers using characterized assays for analysis of proteins, RNA and DNA.
Required tumor biopsies: For patients enrolled in the serial biopsy expansion cohort of Phase Ib Timepoints: Serial biopsies are obtained at two timepoints: 1) Pre-treatment biopsy is obtained at baseline, after all eligibility criteria have been fulfilled, and 2) On-treatment biopsy at approximately 2 weeks after the first administration of both tiragolumab and atezolizumab (i.e., on or between Days 15-21 of Cycle 1).
   An additional optional biopsy may be collected per investigator discretion, preferably at the time of radiographic progression (especially when pseudoprogression is suspected) or at the time of response.
   Serial tumor biopsies are used for evaluation of tumor pharmacodynamic biomarkers and potential predictive biomarkers using characterized assays for analysis of proteins, RNA, and DNA.
Tumor tissue biopsy at progression for crossover from Phase Ia to Phase Ib: For patients being considered for crossover from the Phase Ia to the Phase Ib portion of the study after investigator-assessed radiographic disease progression
   A biopsy at time of progression is required if the sample collection is deemed clinically feasible by the investigator and if the patient has provided written informed consent. Tumor tissue samples consisting of core needle biopsies for deep tumor tissue or lymph nodes or excisional, incisional, punch, or forceps biopsies for cutaneous, subcutaneous, or mucosal lesions are be obtained.
   The biopsy at progression is used for evaluation of tumor pharmacodynamic biomarkers and potential predictive biomarkers using characterized assays for analysis of proteins, RNA, and DNA.
Optional tumor biopsies: For patients who provide specific informed consent to undergo optional biopsies, especially in up to half of the patients enrolled in the expansion cohorts of Phase Ia or Phase Ib
   Patients consenting to optional biopsies provide tumor biopsies at baseline after all eligibility criteria have been fulfilled, and approximately 2 weeks after the first administration of tiragolumab as a single agent (Phase Ia) or approximately 2 weeks after the first administration of tiragolumab and atezolizumab (Phase Ib) (i.e., on or between Days 15-21 of Cycle 1).
   For patients with confirmed, prolonged CR and/or PR (e.g., approximately 1 year in duration) who have an accessible residual mass, a biopsy of that residual mass is recommended to assess for viable tumor cells (vs. fibrotic or necrotic tissue).
   Tumor tissue samples can be collected using core needle, punch, excisional biopsy (as specified above), or forceps biopsy. Please refer to the sample specifications described above for mandatory tumor biopsies for further guidance pertinent to the chosen biopsy technique. The optional tumor biopsies are used for evaluation of tumor pharmacodynamic biomarkers and potential predictive biomarkers using characterized assays for analysis of proteins, RNA, and DNA.
Sample specifications: For all patients in Phase Ia and Phase Ib, the required and optional biopsies are to meet the following sample requirements
   Tumor tissue samples consisting of core needle biopsies of deep tumor tissue or lymph nodes or excisional, incisional, punch, or forceps biopsies of cutaneous, subcutaneous, or mucosal lesions are obtained. Fine-needle aspirates, cell pellets from effusions or ascites, lavage samples, and bone biopsies are not permitted. Target lesions considered for core needle biopsies are deemed suitable for retrieval of at least three cores at a given timepoint (minimum diameter of 18-gauge). If possible, successive passes through the same lesion are to be ≥1 cm apart.
   If multiple lesions are available, it is preferable to obtain the on-treatment biopsy from the same lesion (or organ) as the pretreatment biopsy, if feasible, to avoid introduction of heterogeneity related to site of metastasis.
   RECIST target lesions are not to be biopsied.
   Tissue samples and their derivatives (such as DNA and RNA) are stored indefinitely or until used up or the patient requests destruction of their samples.
Use of remaining samples for all patients in Phase Ia and Phase Ib
   If a patient undergoes a medically indicated procedure (i.e., bronchoscopy, esophagogastroduodenoscopy, colonoscopy, etc.) any time during the course of the study that yields tumor tissue or other tissue that may inform the activity (whether anti-tumor or auto-immune) of tiragolumab as a single agent (Phase Ia) or tiragolumab in combination with atezolizumab with or without chemotherapy (Phase Ib), any remaining samples or a portion of the sample not necessary for medical diagnosis (body fluid samples or leftover tumor tissue) may be obtained for exploratory analysis. Patients must provide specific informed consent in order for these samples to be collected and evaluated.

The remaining tumor samples are used for evaluation of tumor pharmacodynamic biomarkers and potential predictive biomarkers using characterized assays for analysis of proteins, RNA, and DNA.

Electrocardiograms

Triplicate ECG recordings are obtained at specified timepoints for the dose-escalation cohorts and backfill cohorts of tiragolumab alone (Phase Ia) and single ECG recordings for all other patients. ECGs acquired on different days are as closely time-matched as feasible. Three interpretable ECG recordings (e.g., without artifacts) must be obtained at each timepoint (±5 minutes). The average of the three readings is used to determine ECG intervals (e.g., PR, QRS, QT). Additional ECGs may be obtained at unscheduled timepoints as clinically indicated.

All ECG recordings must be performed using a standard high-quality, high-fidelity digital electrocardiograph machine equipped with computer-based interval measurements. Lead placement is as consistent as possible. ECG recordings must be performed after the patient has been resting in a supine position for at least 10 minutes. All ECGs are to be obtained prior to other procedures scheduled at that same time (e.g., vital sign measurements, blood draws) and are not to be obtained within 3 hours after any meal. If following this guidance may be challenging for patients, the time between meal intake and the ECG recording may be shortened to 2.5 hours or to institutional standards as long as the same minimum time is maintained for all timepoints. Circumstances that may induce changes in heart rate, including environmental distractions (e.g., television, radio, conversation) are to be avoided during the pre-ECG resting period and during ECG recording.

For safety monitoring purposes, the investigator must review, sign, and date all ECG tracings. The overall assessment of the ECG as normal or abnormal, with or without clinical significance, are recorded. Paper or electronic copies of ECG tracings are kept as part of the patient's permanent study file at the site.

If at a particular postdose timepoint, the mean QT interval corrected through use of Fridericia's formula (QTcF) is >500 ms and/or >60 ms longer than the baseline value, another ECG must be recorded, ideally within the next 5 minutes, and ECG monitoring continues until QTcF has stabilized on two successive ECGs. Standard-of-care treatment may be instituted per the discretion of the investigator. If a PK sample is not scheduled for that timepoint, an unscheduled PK sample is obtained. The investigator evaluates the patient for potential concurrent risk factors (e.g., electrolyte abnormalities, co-medications known to prolong the QT interval, severe bradycardia).

Cancer-Related Procedures

Collection of cancer-related medical, surgical, and radiation procedures begins on Day 1 and be performed throughout the treatment period and during the survival follow-up period in both the Phase Ia and Phase Ib portions of the study.

Anti-Drug Antibody Testing

Tiragolumab and/or atezolizumab may elicit an immune response against itself. Validated screening, confirmatory, and titer assays are employed to detect ADAs at multiple timepoints before, during, and after treatment with tiragolumab as a single agent in Phase Ia or after treatment with the combination of tiragolumab and atezolizumab with or without chemotherapy in Phase Ib. The ADA response is correlated with relevant clinical endpoints to understand its clinical significance. Additional ADA assays (e.g., neutralizing antibody assay) may be employed to further characterize the ADA response.

U. Patient and Study Treatment Discontinuation

Patient Discontinuation

Patient discontinuation from the study is distinguished from study treatment discontinuation and occurs when the patient dies, is lost to follow-up, or withdraws consent to be followed.

Patients have the right to voluntarily withdraw from the study at any time for any reason. In addition, the investigator has the right to withdraw a patient from the study at any time. Reasons for withdrawal from the study may include, but are not limited to, the following:
Patient withdrawal of consent at any time
Any medical condition that the investigator determines may jeopardize the patient's safety if he or she continues in the study
Investigator determines it is in the best interest of the patient

Every effort is made to obtain information on patients who withdraw from the study. The primary reason for withdrawal from the study or from follow-up is documented. However, patients are not followed for any reason after consent has been withdrawn. Patients who withdraw from the study are not replaced.

Study Treatment Discontinuation

Patients must discontinue study treatment if they experience any of the following:
Symptomatic deterioration (i.e., uncontrollable pain secondary to disease or unmanageable ascites, etc.) attributed to disease progression as determined by the investigator after an integrated assessment of all the radiographic data, biopsy results, and clinical status.
Intolerable toxicity related to study treatment, including development of an immune-mediated adverse event, determined by the investigator to be unacceptable given the individual patient's potential response to therapy and the severity of the event
Any medical condition that the investigator determines may jeopardize the patient's safety if he or she continues on study treatment
Use of a non-protocol systemic anti-cancer therapy
Pregnancy

Patients have the right to voluntarily withdraw from study treatment at any time for any reason. In addition, the investigator has the right to withdraw a patient from study treatment at any time. Reasons for withdrawal from study treatment may include, but are not limited to, the following:
Investigator determines it is in the best interest of the patient
Patient non-compliance

Patients who discontinue study treatment primarily for reasons other than disease progression continue tumor assessments, and all patients who discontinue study treatment continue to be followed for survival every 3 months unless consent is withdrawn.

Patients who discontinue study treatment are asked to return to the clinic for a treatment discontinuation visit at ≤30 days after the last administration of study treatment.

The visit at which a response assessment shows PD, which results in discontinuation of tiragolumab (Phase Ia) or discontinuation of the combination of tiragolumab and atezolizumab, tiragolumab and atezolizumab with bevacizumab, or tiragolumab and pembrolizumab (Phase Ib), may be used as the treatment discontinuation visit as applicable, in which case all assessments associated with the treatment discontinuation visit are be performed at that time.

Following study treatment discontinuation, all patients are followed for survival and subsequent anti-cancer therapy. Survival and subsequent anti-cancer therapy follow-up information is collected via telephone calls, patient medical records, and/or clinic visits approximately every 3 months until death, loss to follow-up, or study termination unless the patient requests to be withdrawn from follow-up. Information on subsequent anti-cancer therapies includes systemic therapies (e.g., chemotherapy, targeted therapy, hormonal therapy, or CIT), surgery (e.g., resection of metastatic disease), and radiation procedures (e.g., radiotherapy to a tumor lesion).

If the patient withdraws from the study, the site's staff may use a public information source (e.g., county records) to obtain information about survival status only.

V. Analysis

Objectives and Endpoints

This study evaluates the safety, PK, pharmacodynamics, and preliminary anti-tumor activity of tiragolumab when administered as a single agent (Phase Ia) or in combination with atezolizumab or with other anti-cancer therapies (Phase Ib) in patients with locally advanced or metastatic tumors. Specific objectives and corresponding endpoints for the study are outlined in Table 36.

TABLE 36
Objectives and Corresponding Endpoints for Phase la and Phase lb

| | |
|---|---|
| Objectives | Corresponding Endpoints |

Safety Objective (Primary Study Objective):

| | |
|---|---|
| To evaluate the safety and tolerability of | Incidence and nature of DLTs |
| tiragolumab when administered as a | Incidence, nature, and severity of adverse |
| single agent (Phase la) or in combination | events graded according to NCI CTCAE v4.0 |
| with atezolizumab (Phase lb), including | Change from baseline in targeted vital signs |
| estimation of the maximum tolerated dose | Change from baseline in targeted clinical |
| (MTD) and the recommended Phase II | laboratory test results, including ECGs |
| dose (RP2D) and characterization of | Number of cycles received and dose intensity |
| dose-limiting toxicities (DLTs) | Incidence of anti-tiragolumab antibodies (Phase |
| To evaluate the safety and tolerability of | la or Phase lb) and/or anti-atezolizumab |
| tiragolumab in combination with | antibodies (Phase lb) and the potential |
| atezolizumab and chemotherapy | correlation with PK, pharmacodynamic, safety, |
| To evaluate the safety and tolerability of | and preliminary efficacy parameters |
| tiragolumab in combination with | |
| atezolizumab and bevacizumab | |
| To evaluate the safety and tolerability of | |
| tiragolumab in combination with | |
| pembrolizumab | |

Pharmacokinetic Objective:

| | |
|---|---|
| To characterize the pharmacokinetic (PK) | Serum concentration of tiragolumab |
| profile of tiragolumab administered as a | administered as a single agent (Phase la) or in |
| single agent (Phase la) or in combination | combination with atezolizumab (Phase lb) with |
| with atezolizumab (Phase lb) | or without chemotherapy at specified timepoints |
| To characterize the PK profile of | for the following parameters: |
| atezolizumab administered in combination | Area under the concentration-time curve |
| with tiragolumab with and without | (AUC) |
| chemotherapy (Phase lb only) | Maximum serum concentration (Cₘₐₓ) |
| To characterize the PK profile of | Minimum serum concentration (Cₘᵢₙ) |
| capecitabine, cisplatin, carboplatin, | Clearance (CL) |
| pemetrexed, paclitaxel, and etoposide | Volume of distribution at steady-state (Vₛₛ) |
| when administered in combination with | Other parameters such as accumulation ratio, |
| atezolizumab and tiragolumab (Phase lb | half-life, and dose proportionality may also be |
| expansion cohorts) | calculated |
| Serum | Cₘₐₓ and Cₘᵢₙ of atezolizumab |
| | Plasma concentrations of capecitabine, cisplatin, |
| | carboplatin, pemetrexed, paclitaxel, and |
| | etoposide at specified timepoints |

Activity Objective:

| | |
|---|---|
| To make a preliminary assessment of the | Objective response, defined as a complete |
| anti-tumor activity of tiragolumab as a | response (CR) or partial response (PR) per |
| single agent (Phase la) or in combination | Response Evaluation Criteria in Solid Tumors |
| with atezolizumab with and without | (RECIST) v.1.1, as determined by the |
| chemotherapy (Phase lb) in patients with | investigator and confirmed by repeat |
| locally advanced or metastatic tumors | assessment ≥4 weeks after initial |
| To make a preliminary assessment of the | documentation. |
| anti-tumor activity of tiragolumab in | Duration of objective response (DoR), defined |
| combination with atezolizumab and | as the time from the first occurrence of a |
| bevacizumab | documented objective response to the time of |
| To make a preliminary assessment of the | the first documented disease progression or |
| anti-tumor activity of tiragolumab in | death from any cause, whichever occurs first, |
| combination with pembrolizumab | per RECIST v1.1 as determined by the |
| | investigator |
| | PFS, defined as the time from the first study |
| | treatment to the first occurrence of progression |
| | or death from any cause, whichever occurs first, |
| | per RECIST v.1.1 as determined by the |
| | investigator |
| | Overall survival (OS), defined as the time from |
| | first study treatment to death from any cause |

Immunogenicity Objective:

| | |
|---|---|
| To characterize the immunogenic | Prevalence of ADAs to tiragolumab at baseline and |
| potential of tiragolumab administered | the incidence of ADAs to tiragolumab during the |
| as a single agent by measuring | study |
| anti-tiragolumab antibodies and | Prevalence of ADAs to atezolizumab at baseline and |
| assessing their relationship with other | the incidence of ADAs to atezolizumab during the |
| outcome measures (Phase la) or | study |
| tiragolumab in combination with | |
| atezolizumab with and without | |
| chemotherapy by measuring | |
| anti-tiragolumab and/or | |
| anti-atezolizumab antibodies and | |
| assessing their relationship with other | |
| outcome measures (Phase lb) | |

Exploratory Biomarker Objectives:

| | |
|---|---|
| To make a preliminary assessment of | Changes in immune infiltrates, immune-related gene |
| biomarkers that might act as | expression, and/or TIG IT-related gene expression in |
| pharmacodynamic indicators of | tumor tissue prior to and during study treatment |
| activity of tiragolumab administered as | |
| a single agent (Phase la) or in | |
| combination with atezolizumab | |
| (Phase lb) with and without | |
| chemotherapy in patients with locally | |
| advanced or metastatic tumors | |
| To make a preliminary assessment of | Levels of TIG IT, PD-Li, and/or other candidate |
| biomarkers that might act as | predictive markers in tumor tissue |
| predictors of anti-tumor activity of | Changes in blood biomarkers, including cytokines |
| tiragolumab administered as a | and immune cells, prior to and during study |
| single agent (Phase la) or in | treatment |
| combination with atezolizumab | Biomarker measurement by RNA sequencing and |
| (Phase lb) with and without | whole genome sequencing (WGS) |
| chemotherapy or in combination with | |
| pembrolizumab or bevacizumab in | |
| patients with locally advanced or | |
| metastatic tumors | |

Activity Analyses

The analyses described below are based on the definitions of objective response according to RECIST v1.1.

Response assessment data, duration of objective response, PFS, and OS are listed for all treated patients by dose level or tumor type, when appropriate.

Objective Response Rate

The analysis of ORR includes patients in the Phase Ia or Phase Ib study who received any amount of the study treatment and have measurable disease at baseline. Objective response is defined as a CR or PR, as determined by investigator assessment and confirmed by repeat assessment ≥4 weeks after initial documentation. Patients with missing baseline or no response assessments are classified as non-responders. Objective response rate is estimated and summarized by tumor type and by dose, if applicable.

Duration of Response

Among patients with an objective response, duration of objective response is defined as the time from the initial complete or partial response to the time of disease progression or death, whichever occurs first. For patients who do not die or experience disease progression before the end of the study or who are lost to follow-up, duration of objective response is censored at the day of the last tumor assessment.

Progression-Free Survival

The analyses of PFS include patients who have received any amount of study treatment. PFS is defined as the time from the first day of study treatment until documented disease progression or death, whichever occurs first. For patients who do not have documented PD or death before the end of the study or who are lost to follow-up, PFS is censored at the day of the last tumor assessment. For patients without any post-baseline tumor assessments, PFS is censored at the first day of study treatment.

Overall Survival

The analyses of OS include patients who have received any amount of study treatment. OS is defined as the time from the first dose of any study treatment to the time of death from any cause on-study. For patients who do not die before the end of the study or who are lost to follow-up, OS is censored at the date last known to be alive.

Safety Analyses

Safety is assessed through summaries of DLTs, adverse events, changes in laboratory test results, changes in vital signs and ECGs, and exposure to any study treatment (tiragolumab, atezolizumab, bevacizumab, pembrolizumab, carboplatin, cisplatin, pemetrexed, paclitaxel, capecitabine, etoposide). All patients who receive any amount of study treatment are included in the safety analyses.

Verbatim descriptions of adverse events are mapped to thesaurus terms. Adverse event data are listed by study site, dose cohort or tumor type as appropriate, patient number, and study day. Events occurring on or after treatment on Day 1 are summarized by mapped term, appropriate thesaurus level, and NCI CTCAE v4.0 grade. In addition, serious adverse events, including deaths, are listed separately and summarized.

Adverse events leading to treatment discontinuation are listed. Patients who withdraw from the study prior to completing the DLT assessment window for reasons other than a DLT are considered non-evaluable for DLT and MTD assessments.

Relevant laboratory, vital signs, and ECG data are displayed by time, with NCI CTCAE Grade 3 and Grade 4 values identified, where appropriate. Incidence of ADA response (presence of serum anti-tiragolumab or anti-atezolizumab) and the potential correlation with PK, pharmacodynamic, and safety parameters may be assessed.

Pharmacokinetic Analyses

In the Phase Ia study, individual and mean serum tiragolumab concentration versus time data are tabulated and plotted by dose level. The pharmacokinetics of tiragolumab is summarized by estimating total AUC, Cₘₐₓ, Cₘᵢₙ, total CL, Vₛₛ, and terminal half-life (as appropriate for data collected). Estimates for these parameters are tabulated and summarized (mean, standard deviation, and coefficient of variation). Interpatient variability and drug accumulation are evaluated.

In the Phase Ib study, serum tiragolumab and atezolizumab concentration data (Cₘₐₓ and Cₘᵢₙ) are tabulated and summarized by dose level for each cycle where collected. Descriptive statistics include mean, median, standard deviation, and range, as appropriate. Other PK parameters may be determined and summarized as data warrant. Data may be compared with historical data, as these results provide preliminary information on whether tiragolumab or atezolizumab PK are altered by co-administration of the other agent.

In the Phase Ib chemotherapy expansion cohorts, serum concentrations of tiragolumab and atezolizumab and plasma concentrations of carboplatin, cisplatin, pemetrexed, paclitaxel, capecitabine, and etoposide are collected. The concentrations of tiragolumab, atezolizumab, carboplatin, cisplatin, pemetrexed, paclitaxel, capecitabine, and etoposide are summarized using descriptive statistics as described above. Data are compared with historical data, as these results provide preliminary information on whether tiragolumab, atezolizumab, carboplatin, cisplatin, pemetrexed, paclitaxel, capecitabine, and etoposide PK is altered by co-administration of the other agent.

Pharmacodynamic analyses include assessments of pharmacodynamic biomarkers in both tumor tissue and blood when available. Additional PK and pharmacodynamic analyses areconducted as appropriate. Potential adjustment of biomarker sampling times may be applied if needed, based on observed biomarker response in earlier patients.

Immunogenicity Analyses

Patients are considered to have treatment-induced ADA responses if they are ADA negative at baseline and then develop an ADA response following study drug administration. Patients are considered to have treatment-enhanced ADA responses if they are ADA positive at baseline and the titer of one or more post-baseline samples is at least 4-fold greater (i.e., ≥0.60 titer units) than the titer of the baseline sample. Patients are considered to be negative for ADAs if they are ADA negative at all timepoints. Patients are considered to be treatment unaffected if they are ADA positive at baseline but do not have any post-baseline samples with a titer that is at least 4-fold greater than the titer of the baseline sample.

ADA results for tiragolumab and atezolizumab are summarized and listed by patient and cycle for the Phase Ia and Phase Ib portions of the study.

Interim Analyses in the Expansion Cohorts of Phase Ia and Phase Ib

Interim analyses are conducted by the IMC for each expansion cohort in Phase Ia and Phase Ib to guide potential early stopping of enrollment when there is no evidence of activity. After approximately 20 patients enrolled in an expansion cohort have completed 1-2 tumor assessments, the IMC meets to conduct an interim analysis. If the interim analysis suggests that activity as manifested by objective tumor response with tiragolumab (Phase Ia) or with tiragolumab in combination with atezolizumab with and without chemotherapy, or tiragolumab in combination with atezolizumab and bevacizumab, or tiragolumab in combination with pembrolizumab (Phase Ib) is below a threshold of interest for that patient group (e.g., if no responses are observed in the first 20 patients), the IMC may recommend that enrollment into the expansion cohort be stopped.

For example, if the true ORR is 5%, there is an approximate 35% probability that no responses are observed in the first 20 patients, and if the true ORR is 10%, there is an approximate 12% probability that no responses are observed in the first 20 patients.

The IMC may also recommend that enrollment continue in that expansion cohort if there is evidence of activity or if not enough specific patients had enrolled to allow adequate evaluation (e.g., patients with specific tumor type, specific tumor PD-L1 and/or TIGIT expression status, and/or specific CIT history).

In all cases, decisions to stop enrollment into the expansion cohorts based on futility are made following the IMC recommendation by the Medical Monitor in consultation with the study investigators. The Medical Monitor may also request additional ad-hoc meetings of the IMC to review ongoing data in each expansion cohort in Phase Ia or Phase Ib.

W. Summary of Clinical Studies for Tiragolumab

Preliminary clinical data are available from this ongoing study (GO30103) evaluating tiragolumab in patients with locally advanced or metastatic tumors. As of the clinical cutoff date of 2 Dec. 2019, there were 190 enrolled patients in Study GO30103, with 42 patients enrolled in the Phase Ia portion and 171 patients enrolled in the Phase Ib portion, including 23 patients that crossed over from Phase Ia following disease progression.

An ongoing Phase II study (GO40290, also called CITYSCAPE) is evaluating the efficacy and safety of tiragolumab and atezolizumab compared with placebo plus atezolizumab in chemotherapy-naive patients with locally advanced unresectable or metastatic PD-L1-selected NSCLC, excluding patients with a sensitizing epidermal growth factor receptor (EGFR) mutation or ALK translocation.

Clinical Pharmacokinetics and Immunogenicity

As of the data cutoff date of 2 Dec. 2019, a preliminary PK analysis was conducted based on available data (2-1200 mg of tiragolumab administered every 3 weeks in the Phase Ia portion and 2-1200 mg of tiragolumab administered every 3 weeks in combination with 1200 mg atezolizumab administered every 3 weeks in the Phase Ib portion of Study GO30103) using standard non-compartmental PK methods. The pharmacokinetics of tiragolumab in combination with atezolizumab appeared to be consistent with the pharmacokinetics of tiragolumab administered as a single agent. Preliminary population-PK analyses show that the tiragolumab exposures increased approximately dose proportionally following IV administration at doses ranging from 100 mg to 1200 mg every 3 weeks as monotherapy or in combination with 1200 mg atezolizumab every 3 weeks. Preliminary population PK analysis estimated tiragolumab clearance at 0.28 L/day with a linear drug elimination half-life of approximately 15 days. Development of anti-drug antibodies (ADAs) to tiragolumab was observed in 3 of 154 evaluable patients (1.9%) in the Phase Ib portion of Study GO30103. Preliminary data suggest that there was no apparent impact of tiragolumab ADAs on PK. However, the small number of positive ADA patients were not adequate to assess the impact of ADAs on the pharmacokinetics of tiragolumab.

Clinical Safety

As of the data cutoff date of 2 Dec. 2019, safety data were available for the 42 patients in the Phase Ia portion of Study GO301 03. Commonly reported adverse events in Phase Ia (reported in 10% of all patients), regardless of relationship to tiragolumab, included fatigue, constipation, vomiting, decreased appetite, anemia, cough, dyspnea, headache, infusion-related reaction (IRR), malignant neoplasm progression, nausea, and pruritus. Commonly reported adverse events (reported in ≥10% of patients) related to tiragolumab included fatigue and IRR.

Grade ≥3 adverse events (on the basis of National Cancer Institute Common Terminology Criteria for Adverse Events, Version 4.0 [NCI CTCAE v4.0]), regardless of attribution to the study drugs, were reported in 16 patients (38.1%) in the Phase Ia portion of Study GO30103, with 3 patients (7.1%) experiencing treatment-related Grade ≥3 adverse events. Grade ≥3 adverse events considered related to tiragolumab by the investigator included amylase increased, blood creatinine increased, hepatic failure (described below), IRR, and rash.

As of the data cutoff date of 2 Dec. 2019, 171 patients were enrolled in the Phase Ib portion of Study GO30103, including 23 patients that crossed over from Phase Ia following disease progression. Of the 171 patients treated in the Phase Ib, 163 patients (95.9%) experienced at least one adverse event, including 110 patients (64.7%) who experienced at least one treatment-related adverse event. Commonly reported adverse events in Phase Ib (reported in ≥10% of all patients), regardless of relationship to tiragolumab, included pruritus, pyrexia, anemia, rash, malignant neoplasm progression, constipation, decreased appetite, diarrhea, cough, asthenia, vomiting and fatigue. Commonly reported adverse events (reported in ≥10% of patients) related to tiragolumab and/or atezolizumab included pruritus and rash.

Grade ≥3 adverse events (on the basis of NCI CTCAE v4.0), regardless of attribution to the study drugs, were reported in 91 patients (53.5%) in the Phase Ib portion of Study GO30103, with 21 patients (12.4%) experiencing treatment-related Grade ≥3 adverse events. Grade ≥3 adverse events considered related to tiragolumab and/or atezolizumab by the investigator included lymphocyte count decreased, lipase increased, lymphopenia, amylase increased, adrenal insufficiency, asthenia, cardiac tamponade, headache, hyperglycemia, hyperlipasemia, hypertransaminasasmia, pneumonitis, rash pruritic, rhabdomyolysis, diabetes mellitus, diabetic ketoacidosis, and lipase.

A fatal case of hepatic toxicity culminating in fulminant liver failure occurred in a patient enrolled in the Phase Ia monotherapy expansion cohort of Study GO30103. At screening, the patient's transaminases were within normal limits. On C1 D1 prior to dosing, the AST and ALT had risen to Grade 1. During tiragolumab administration on C1 D1, the patient developed an infusion reaction that responded to dose interruption and standard treatment; the patient received approximately 733 mg of the intended 1200 mg dose of tiragolumab. At Study Day 8, the ALT/AST had increased to Grade 3, associated with Grade 3 rash; the patient was treated with prednisone 60 mg daily. By Study Day 12, the rash was improving but the AST and ALT were Grade 4 (with normal bilirubin and PT-international normalization ratio (INR)) and a second immunosuppressive agent (azathioprine) was added. The patient progressively worsened despite the use of the two immunosuppressive agents and developed overt liver failure with elevated bilirubin and progressive hepatic encephalopathy, ultimately leading to death on Study Day 30. Multiple alternative etiologies of hepatitis were excluded including viral (HBV, HCV), concomitant medications, or anatomic abnormalities. A liver biopsy performed on Study Day 16 demonstrated confluent submassive necrosis with scant inflammatory infiltrate interpreted as toxic hepatitis.

Following this event, analysis of safety data indicated no significant changes to the adverse event findings and no new significant adverse events were noted for the subjects dosed in the Phase Ia and Phase Ib portions of Study GO30103.

As of the data cutoff date of 30 Jun. 2019, in the Phase II blinded Study GO40290, 135 safety-evaluable patients were administered either tiragolumab (600 mg every 3 weeks) in combination with atezolizumab or placebo in combination with atezolizumab. The safety profile of tiragolumab and atezolizumab was similar to that of placebo and atezolizumab for all-grade adverse events (98.5% vs. 95.6%), Grade ≥3 AEs (41.8% vs. 44.1%), serious AEs (34.3% vs. 35.3%), and AEs leading to treatment discontinuation (7.5% vs. 10.3%). Adverse events of special interest were reported in 47.8% of patients in the tiragolumab and atezolizumab arm and in 32.4% of patients in the placebo plus atezolizumab arm. The updated data from December 2019 showed similar safety profile.

One fatal case of EBV reactivation and possible secondary hemophagocytic lymphohistiocytosis (HLH) was reported in a 69-year-old male in Taiwan with metastatic lymphoepithelioma-like carcinoma subtype of NSCLC (PD-L1 positive), which can be associated with EBV infection in Asian patients. At screening, the patient had a positive serum Epstein-Barr nuclear antigen (EBNA) antibody test but did not have clinical signs of active EBV infection as confirmed by the treating investigator. On Day 1 of Cycle 1, the patient received both study drugs, atezolizumab and tiragolumab, and tolerated treatment well. On Day 10, the patient developed a fever that was believed to be due to an upper respiratory tract infection, which was treated with an antibiotic. On Day 1 of Cycle 2, the patient received atezolizumab and developed a Grade 2 fever 20 minutes after the infusion. Because the fever was presumed to be an atezolizumab IRR, tiragolumab was not administered for Cycle 2. The patient was hospitalized for the fever, but a workup for infectious cause was negative. Upon resolution of the fever, the patient was discharged on Day 8 of Cycle 2. On Day 15 of Cycle 2, the patient was hospitalized again for intermittent fevers and was transferred to the intensive care unit as he developed shock with hypotension, metabolic acidosis, acute kidney injury, pancytopenia, coagulopathy, bleeding, altered mental status, transaminitis, hyperbilirubinemia, and respiratory failure. The patient had high EBV titers in the blood, and a bone marrow biopsy was consistent with HLH. Cultures for other infectious etiologies were negative. Despite treatment with steroids and multiple immunosuppressants (tocilizumab, hydroxychloroquine, and etanercept), the patient worsened and died on Day 31 of Cycle 2. In total, the patient had received one dose of tiragolumab (at Cycle 1) and two doses of atezolizumab (at Cycles 1 and 2). While the exact cause of the events is unknown at the time of this amendment and investigations are ongoing, the available information suggests that this patient had a rare histological subtype of NSCLC and suspected chronic active EBV infection that reactivated with administration of tiragolumab and atezolizumab. It appears that the patient developed secondary HLH, given the temporal relationship between study drug exposure and the patient's clinical course. Since this index case of EBV reactivation in a patient with pulmonary lymphoepithelioma-like carcinoma, the safety data has been carefully reviewed, and no other cases of viral reactivation have been observed in the Phase II study (GO40290) or in the current Phase I study (GO30103).

As of the data cutoff date of 2 Dec. 2019, 42 patients have been treated with tiragolumab single agent. Patients receiving single-agent tiragolumab have experienced IRRs. In the Phase Ia, 5 patients (12%) experienced IRRs. Signs and symptoms associated with IRR's were generally mild and included pyrexia, chills, hot flush, hypertension, lymphoadenopathy, nausea, myalgia, and skin lesion. All cases of IRRs were successfully treated with supportive measures. Due to IRRs occurring during and after infusion with single-agent tiragolumab in 12% of patients, IRRs were moved from a potential to an identified risk. While there are no additional identified risks with tiragolumab, engagement of the TIGIT co-inhibitory pathway may increase the risk of autoimmune inflammation when tiragolumab is administered as a single agent. Therefore, tiragolumab as a single-agent may cause adverse events similar to atezolizumab. Such immune-mediated adverse events have been well-characterized for other immune checkpoint inhibitors such as anti-CTLA-4 and anti-PD-L1/PD-1.

This is the first clinical evaluation of tiragolumab in combination with an anti-PD-L1/PD-1 agent. While clinical evaluation of tiragolumab in combination with atezolizumab is limited and not all risks are known, as of 2 Dec. 2019, over 200 patients have been treated with tiragolumab in combination with atezolizumab. While there are currently no unique identified risks for tiragolumab given with atezolizumab, engagement of the TIGIT co-inhibitory pathway may increase the risk of some immune reactions including HLH and MAS when tiragolumab is administered in combination with atezolizumab. Therefore, when given with atezolizumab, tiragolumab may exacerbate atezolizumab-related adverse events, or may have non-overlapping toxicities with atezolizumab.

The largest clinical experience to date with the combination of complementary modulators of adaptive immunity is derived from trials of ipilimumab in combination with nivolumab. Another anti-TIGIT antibody MK-7684 (vibostolimab) when combined with pembrolizumab showed acceptable safety profile and promising anti-tumor activity in patients with advanced tumors. On the basis of these data, it is anticipated that combination immune-mediated adverse events will be amenable to monitoring and manageable in the clinical setting, although the frequency and severity of immune-mediated adverse events may be increased with the combination when compared with either single-agent inhibitor.

Clinical Efficacy

As of the data cutoff date of 2 Dec. 2019, no objective responses have been reported for patients enrolled in Phase Ia portion of Study GO30103; best response was stable disease (SD) in 8 of 42 patients with on-study tumor assessments. In the Phase Ib portion of the study, objective responses, including complete responses (CRs) in 4 of 171 patients and partial responses (PR) in 23 of 171 patients, have been observed in patients who are naive to cancer immunotherapy (CIT) with multiple tumor types, including NSCLC, HNSCC, and triple-negative breast cancer.

As of the data cutoff date of 30 Jun. 2019, data from the blinded, randomized, Phase II Study GO40290 showed that the combination of tiragolumab and atezolizumab improved ORR and PFS compared to placebo and atezolizumab in the ITT population. ORR for tiragolumab and atezolizumab was 31.3% (95% CI: 19.5 to 43.2) compared to placebo and atezolizumab which was 16.2% (95% CI: 6.7 to 25.7). Investigator-assessed PFS for tiragolumab and atezolizumab was 5.4 months (95% CI: 4.2, not reached) compared to the PFS for placebo and atezolizumab, which was 3.6 months (95% CI: 2.7 to 4.4), with a HR of 0.57 (95% CI: 0.37 to 0.90).

Phase Ib with Tiragolumab and Atezolizumab with Chemotherapy

In multiple, large, randomized, Phase III trials of advanced solid tumors, the addition of anti-PD-L1/PD-1 to standard chemotherapy has improved OS and PFS for patients compared to chemotherapy alone. These trials studied the addition of anti-PD-1 to pemetrexed and platinum-based chemotherapy in nonsquamous NSCLC (Gandhi et al. N Engl J Med 2018; 378:2078-92) and to carboplatin and paclitaxel or nab-paclitaxel in squamous NSCLC (Paz-Ares et al. N Engl J Med 2018; 379:2040-51). These trials also investigated the addition of anti-PD-L1 to carboplatin and etoposide in extensive stage SCLC (Horn et al. N Engl J Med 2018; 379:2220-9) and to nab-paclitaxel in TNBC (Schmid et al. N Engl J Med 2018; 379:2108-21). As a result of these trials, anti-PD-L1/PD-1 in combination with chemotherapies have been approved by the FDA and EMA, for patients with advanced cancer. As nonclinical data suggests that concomitant anti-TIGIT and anti-PD-L1/PD-1 enhances anti-tumor immune responses more than single-agent anti-PD-L1/PD-1 alone, it is hypothesized that the addition of anti-TIGIT to anti-PD-L1/PD-1 combined with chemotherapy may be more efficacious than the addition of anti-PD-L1/PD-1 to chemotherapy.

The safety profiles resulting from combining anti-PD-L1/PD-1 and chemotherapy have generally been consistent with the known toxic effects of each agent as observed in multiple clinical trials of advanced solid tumors (Gandhi et al. N Engl J Med 2018; 378:2078-92; Horn et al. N Engl J Med 2018; 379:2220-9; Paz-Ares et al. N Engl J Med 2018; 379:2040-51; Schmid et al. N Engl J Med 2018; 379:2108-21). Given the similarities in mechanism of action between tiragolumab and atezolizumab, it is anticipated that the safety profile of tiragolumab administered in combination with atezolizumab and chemotherapy may be consistent with the immune-related toxicities of the combined anti-TIGIT and anti-PD-L1 and the toxicities of the individual chemotherapy agents. Therefore, the design and safety management plan for the Phase Ib chemotherapy expansion cohorts incorporate the extensive clinical experience with chemotherapy as well as the considerations described for combining anti-TIGIT with anti-PD-L1 to reduce the potential risks to participating patients.

Phase Ib with Tiragolumab and Atezolizumab with Bevacizumab

A strong scientific rationale and emerging clinical data suggest that combined PD-L1, VEGF, and TIGIT inhibition may be clinically beneficial in a number of tumor types.

In chemotherapy-naive patients with Stage IV NSCLC, results from Study GO29436 (IMpower150) have shown that atezolizumab plus bevacizumab and chemotherapy results in significantly longer OS compared with bevacizumab and chemotherapy alone (Socinski et al. N Engl J Med 2018; 378:2288-301). For patients with inoperable, locally advanced, or metastatic RCC, results from Study WO29637 (IMmotion151) demonstrated improved PFS after treatment with the combination of atezolizumab and bevacizumab compared with sunitinib in a treatment-naive patient population (Motzer et al. J Clin Oncol 2018; 36(6_suppl):578, Rini et al. Lancet 2019; 393:2404-15). The Phase Ib Study GO30140 in frontline HCC found an ORR of 65% in 23 evaluable patients, across etiology, geography, baseline alpha fetoprotein levels, and extrahepatic spread (Stein et al. J Clin Oncol 2018; 36(Suppl 15):4074, Lee et al. Lancet Oncol. 2020 June; 21(6):808-820). Based on these results, this treatment was granted breakthrough designation, and the results were confirmed in a randomized Phase III study, the YO40245 (IMbrave150) study. Study YO40245 (IMbrave150) was designed to evaluate the efficacy and safety of atezolizumab and bevacizumab versus sorafenib in patients with advanced or metastatic HCC who received no prior systemic treatment. The study enrolled 501 patients randomized in a 2:1 ratio. The most recent study results demonstrated a statistically significant and clinically meaningful improvement in PFS and OS. The risk of death was reduced by 42% for the atezolizumab plus bevacizumab arm compared with the sorafenib arm (stratified HR=0.58 (95% CI: 0.42 to 0.79); median OS, NE vs. 13.24 months). Independent Review Facility-assessed PFS per Response Evaluation Criteria in Solid Tumors (RECIST) v1 0.1 demonstrated improvement favoring combination treatment (stratified HR=0.59 (95% CI: 0.47 to 0.76); median PFS, 6.83 vs. 4.27 months) (Cheng et al. Ann Oncol 2019; 30:ix186-ix187, Finn et al. N Engl J Med 2020; 382:1894-1905).

These are examples of potential synergy between atezolizumab and bevacizumab in different tumor types, which supports further exploration of the combination with tiragolumab.

### Example 5. Pharmacokinetics, Pharmacodynamics, Safety, and Efficacy in a Phase Ia/Lb Dose-Escalation Study of the Anti-TIGIT Antibody Tiragolumab as a Single Agent and in Combination with Atezolizumab in Patients with Advanced Solid Tumors

A. Patient Characteristics

The baseline characteristics of patients in the phase Ia and phase Ib portions of the study are shown in Table 37. Both phase Ia and phase Ib patients include heavily pre-treated populations, with 42% of phase Ia patients and 37% of phase Ib patients having greater than or equal to four prior cancer therapies. Phase Ia and phase Ib patients share similar baseline characteristics.

TABLE 37
Baseline Characteristics

| | | | Phase lb: | |
|---|---|---|---|---|
| | | | Tiragolumab + | |
| | Phase la: Tiragolumab | | Atezolizumab | |
| Characteristic, n (%) | (n = 24) | | (n = 49) | |
| Age in years, median (range) | 59.5 | (40-77) | 54.0 | (25-81) |
| Male | 10 | (42%) | 24 | (49%) |
| ECOG PS 1 | 17 | (71%) | 36 | (74%) |

Race

| | | | | |
|---|---|---|---|---|
| White | 15 | (63%) | 30 | (61%) |
| Asian | 7 | (29%) | 13 | (27%) |

Prior cancer therapies

| | | | | |
|---|---|---|---|---|
| 1 | 2 | (8%) | 7 | (14%) |
| 2 | 6 | (25%) | 14 | (29%) |
| 3 | 6 | (25%) | 10 | (20%) |
| ≥4 | 10 | (42%) | 18 | (37%) |

Primary cancer history type

| Colon | 4 | (17%) | 8 | (16%) |
|---|---|---|---|---|
| Rectum | 4 | (17%) | 4 | (8%) |
| Breast | 2 | (8%) | 9 | (18%) |
| Non-small cell lung cancer | — | | 6 | (12%) |
| Head and neck cancer | — | | 4 | (8%) |
| Ovarian | 1 | (4%) | 3 | (6%) |
| Other* | 13 | (54%) | 15 | (31%) |

ECOG = Eastern Cooperative Oncology Group; PS = performance status
*Other includes:
Phase la: endometrial (n = 2), melanoma (n = 2); and appendiceal, bladder, cervical, cholangiocarcinoma, kidney, neuroendocrine, peritoneum, sarcoma, and stomach (n = 1 each)
Phase lb: sarcoma (n = 4), stomach (n = 2), melanoma (n = 2); and anus, appendiceal, bladder, esophagus, GE junction, Merkel cell, and peritoneum (n = 1 each)

Most patients in the phase Ia and phase Ib portions of the study discontinued treatment for progression of disease (42% of phase Ia and 76% of phase Ib). None of the phase Ib crossover patients had a response. Patient disposition is shown in Table 38.

TABLE 38
Patient Disposition

| | | Phase lb: |
|---|---|---|
| | | Tiragolumab + |
| | Phase la: Tiragolumab | Atezolizumab |
| Status, n (%) | (n = 24) | (n = 49) |
| On treatment | 0 | 5 (10%) |
| Discontinued | 24 (100%) | 44 (90%) |
| Progression of disease | 10 (42%) | 37 (76%) |
| Withdrawal | 2 (8%) | 6 (12%) |
| Lost to follow-up | 0 | 1 (2%) |
| Crossover to Phase lb | 12 (50%) | — |

B. Pharmacokinetics and Pharmacodynamics of Tiragolumab

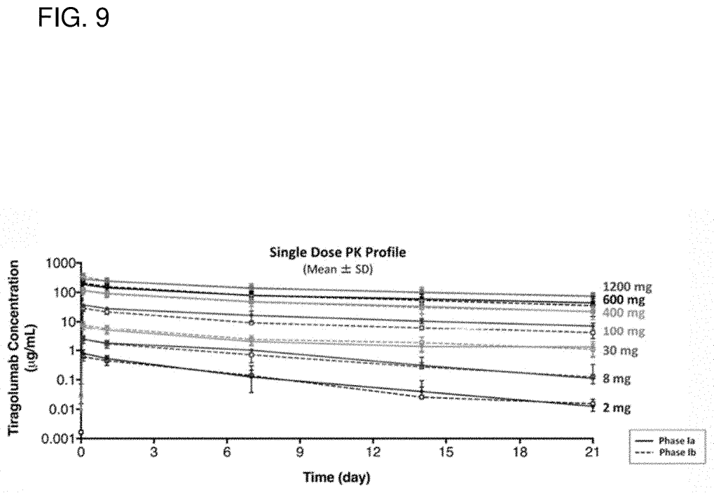

The pharmacokinetics of tiragolumab as a single agent (phase Ia) and in combination with atezolizumab (phase Ib) was measured. The exposure of tiragolumab increased with dose as a single agent and in combination with atezolizumab (FIG. 9). The pharmacokinetics of tiragolumab are not altered in combination with atezolizumab

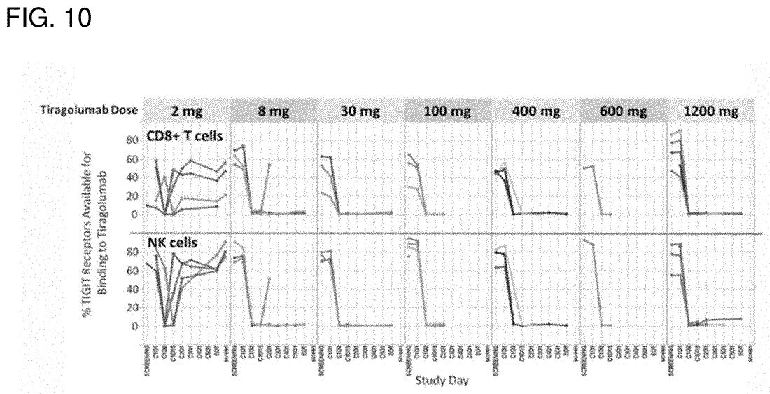

The pharmacodynamics of tiragolumab as a single agent (phase Ia) was measured (FIG. 10). Occupancy of peripheral TIGIT receptors on CD8⁺ T cells and on NK cells were measured at doses of 2 mg, 8 mg, 30 mg, 100 mg, 400 mg, 600 mg, and 1200 mg every three weeks. Complete and sustained occupancy of peripheral TIGIT receptors after day two (CI D2) was observed at tiragolumab doses greater than or equal to 30 mg.

C. Safety

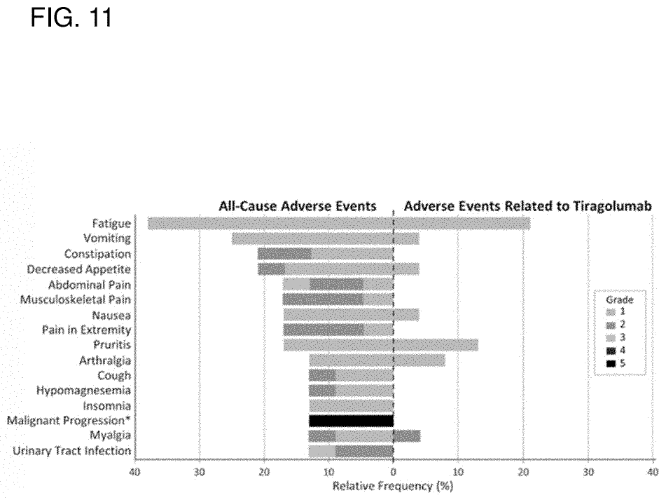

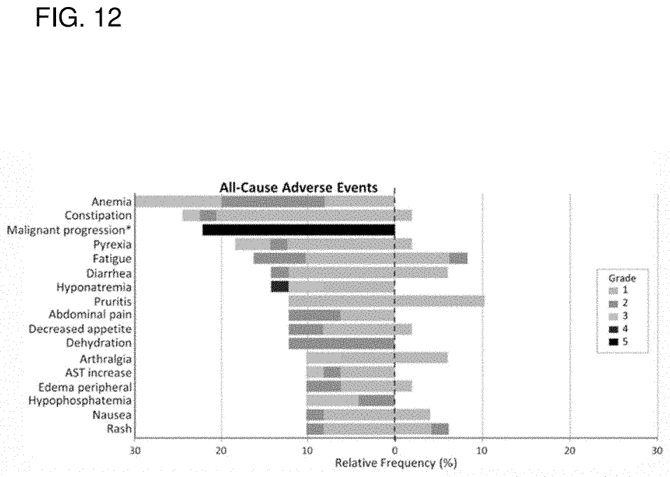

A summary of adverse events is shown in Table 39. The overall rates of grade 3-5 adverse events were low for the phase Ia and phase Ib portions and neither had any grade 5 adverse events. Dose limiting toxicities were not observed for tiragolumab as a single agent or in combination with atezolizumab. All adverse events present in greater than or equal to 10% of patients in phase a and phase Ib are shown in FIGS. 11 and 12, respectively. All immune-mediated adverse events are shown in Table 40. Tiragolumab was well-tolerated as a single agent and in combination with atezolizumab.

TABLE 39
Safety summary of adverse events

| | | Phase lb: |
|---|---|---|
| | Phase la: | Tiragolumab + |
| | Tiragolumab | Atezolizumab |
| | (n = 24) | (n = 49) |
| Any-cause adverse event (AE) | 24 (100%) | 46 (94%) |
| Grade 3-5 adverse event | 6 (25%) | 28 (57%) |
| Related Grade 3-5 adverse events* | 1 (4%) | 2 (4%) |
| Serious adverse events | 6 (25%) | 26 (53%) |
| AE leading to study drug(s) interruption | 4 (17%) | 12 (25%) |
| AE leading to study drug(s) withdrawal** | 0 | 1 (2%) |

*Related AEs in Phase la was Grade 3 blood creatinine increased (n = 1). No Grade 5 AEs were associated with tiragolumab.
Related AEs in Phase lb were Grade 3 hyperlipasemia (n = 1) and Grade 3 lymphocyte count decreased (n = 1). No Grade 5 AEs were associated with tiragolumab and/or atezolizumab.
**One patient withdrew from the Phase lb study for gastrointestinal complaints related to clinical progression.

TABLE 40
All immune-mediated adverse events

| | | Phase lb: |
|---|---|---|
| | | Tiragolumab + |
| | Phase la: Tiragolumab | Atezolizumab |
| | (n = 24) | (n = 49) |
| All immune-mediated AE | 4 (17%) | 29 (59%) |
| Grade 3-5 immune-mediated AE* | 0 | 2 (4%) |
| Infusion-related reaction | 2 (8%) | 4 (8%) |
| Rash | 2 (8%) | 14 (29%) |
| Hepatitis (diagnosis and lab) | 1 (4%) | 10 (20%) |
| Pancreatitis (lab) | 1 (4%) | 1 (2%) |
| Hyperthyroidism | 0 | 4 (8%) |
| Hypothyroidism | 0 | 3 (6%) |
| Anemia | 0 | 1 (2%) |

*No Grade 5 immune-mediated AEs were associated with tiragolumab and/or atezolizumab

D. Efficacy

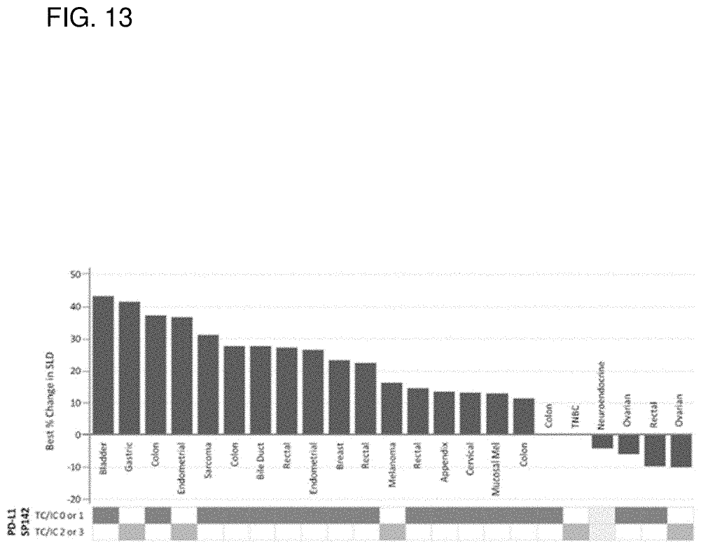

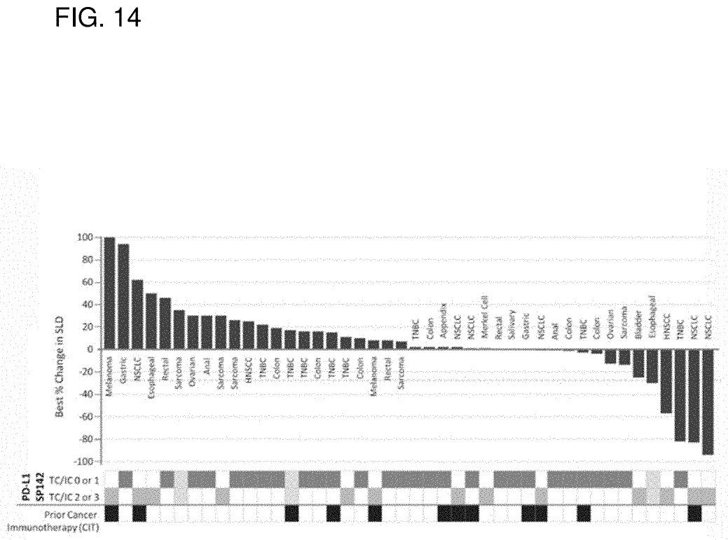

A preliminary efficacy analysis was performed on the phase Ia and phase Ib dose escalation study. In the phase Ia portion, a reduction in tumor size was observed in neuroendocrine, ovarian, and rectal cancers (FIG. 13). Although there were no objective responses in the phase Ia portion of the study, most patients had tumor types not typically responsive to cancer immunotherapy (CIT), had tumors with low PD-L1 expression, or were heavily pre-treated. Objective responses were observed in the phase Ib portion of the study in NSCLC, TNBC, HNSCC, and esophageal cancer types (FIG. 14).

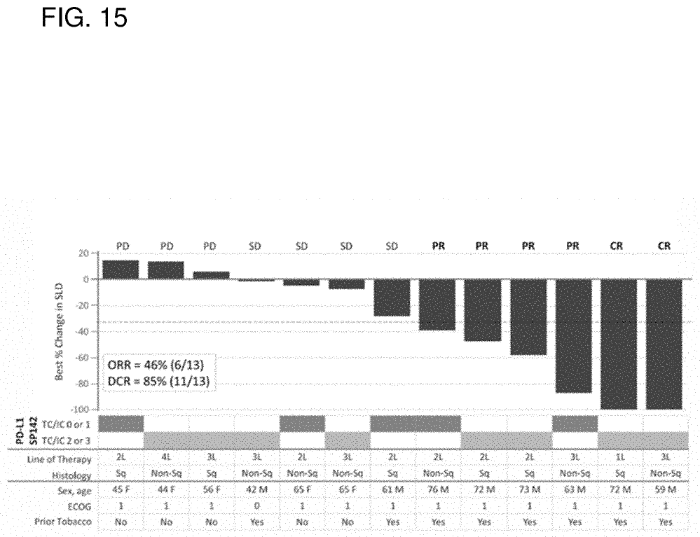

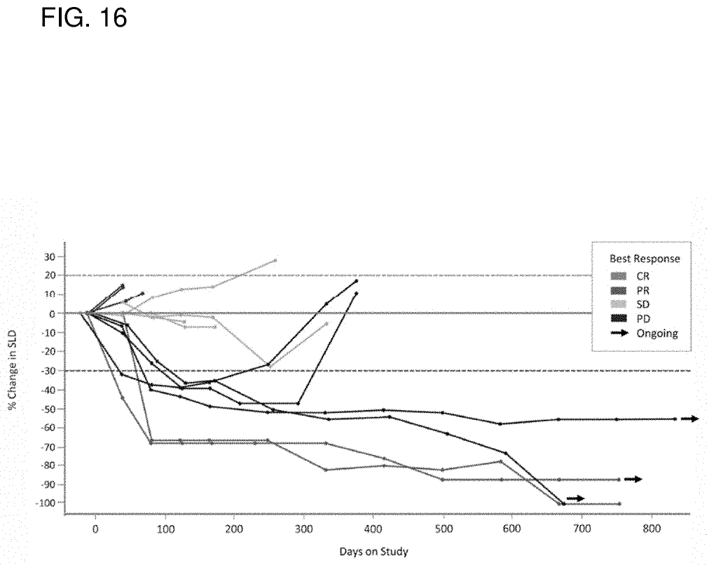

The size of PD-L1-positive NSCLC tumors, including CIT-naïve tumors, were greatly reduced in patients treated with tiragolumab in combination with atezolizumab (FIG. 14). CIT-naïve PD-L1-positive NSCLC treated with tiragolumab in combination with atezolizumab had a 46% overall response rate, which included two complete responses and several responses that show durability (FIGS. 15 and 16).

### Example 6: Atezolizumab Combined with Tiragolumab and Bevacizumab for Advanced Liver Cancer

Liver cancer is the fifth most common cancer and the second most frequent cause of cancer-related death globally, with 854,000 new cases and 810,000 deaths per year. Hepatocellular carcinoma (HCC) is the most prevalent form of primary liver cancer and represents approximately 90% of all primary hepatic malignancies. Less prevalent primary liver cancers include intrahepatic cholangiocarcinoma (iCCA), angiosarcoma, and hepatoblastoma.

Study YO040245 (IMbrave150) is an ongoing, randomized Phase III study evaluating atezolizumab plus bevacizumab versus sorafenib as first-line treatment in patients with advanced or metastatic HCC. This study is the first to demonstrate a statistically significant and clinically meaningful improvement in OS and progression-free survival (PFS) for a novel treatment combination in a head-to-head comparison with sorafenib. At the time of the primary analysis, the risk of death was reduced by 42% for the atezolizumab plus bevacizumab arm compared with the sorafenib arm (stratified hazard ratio [HR]=0.58 [95% CI: 0.42 to 0.79]; p=0.0006; median OS, not estimable [NE] vs. 13.24 months). Independent-Review Facility-assessed PFS per RECIST v1.1 also demonstrated a statistically significant and clinically meaningful improvement favoring the combination treatment (stratified HR=0.59 [95% CI: 0.47 to 0.76]; p<0.0001; median PFS, 6.83 vs. 4.27 months). Overall, the atezolizumab plus bevacizumab combination in HCC was generally well tolerated with manageable toxicities and the safety profile was consistent with the known risks of the individual study treatments and with the underlying disease (Cheng et al. IMbrave150: Efficacy and Safety Results From a Ph 3 Study Evaluating Atezolizumab (atezo)+Bevacizumab (bev) vs Sorafenib (Sor) as First Treatment (tx) for Patients (pts) With Unresectable Hepatocellular Carcinoma (HCC). Proceedings of ESMO Asia 2019: 22-24 Nov. 2019 [cited: 27 Nov. 2019]; Singapore.

This is a Phase Ib/II, open-label, multicenter, randomized umbrella study in patients with locally advanced or metastatic hepatocellular carcinoma (HCC) who have not received prior systemic therapy for their disease.

Patients are randomly assigned to a control arm (atezolizumab plus bevacizumab [Atezo+Bev]) or treatment arm consisting of atezolizumab and bevacizumab in combination with tiragolumab (Atezo+Bev+Tira).

Control Arm (Atezo+Bev)

Patients in the atezolizumab plus bevacizumab (Atezo+Bev) arm receive treatment as outlined in the following table.

TABLE 41
Treatment Regimen for Atezo + Bev Arm

| | Dose, Route, and Regimen |
|---|---|
| Cycle Length | (drugs listed in order of administration) |
| 21 days | Atezolizumab 1200 mg by IV infusion on Day 1 |
| | Bevacizumab 15 mg/kg by IV infusion on Day 1 |

Atezo + Bev = atezolizumab plus bevacizumab.

Atezo+Bev+Tira

Patients in the atezolizumab plus bevacizumab plus tiragolumab (Atezo+Bev+Tira) arm receive treatment as outlined in the following table.

TABLE 42
Treatment Regimen for Atezo + Bev + Tira Arm

| | Dose, Route, and Regimen |
|---|---|
| Cycle Length | (drugs listed in order of administration) |
| 21 days | Atezolizumab 1200 mg by IV infusion on Day 1 |
| | Bevacizumab 15 mg/kg by IV infusion on Day 1 |
| | Tiragolumab 600 mg by IV infusion on Day 1 ^{a} |

Atezo + Bev + Tira = atezolizumab plus bevacizumab plus tiragolumab; IRR = infusion-related reaction.
^{a} On Day 1 of Cycle 1, tiragolumab is administered 60 minutes after completion of the bevacizumab infusion. The interval between subsequent infusions will be 30 minutes if the previous bevacizumab infusion was given without premedication and tolerated without an IRR or 60 minutes if the patient experienced an IRR with the previous bevacizumab infusion.

B. Inclusion Criteria

Patients must meet all of the following criteria:
Age ≥18 years.
ECOG Performance Status of 0 or 1 within 7 days prior to treatment.
Locally advanced or metastatic and/or unresectable HCC with diagnosis confirmed by histology/cytology or clinically by AASLD criteria in cirrhotic patients
   For cirrhotic patients with no histological confirmation of diagnosis, clinical confirmation is required per AASLD criteria.
Child-Pugh class A within 7 days prior to randomization
Disease that is not amenable to curative surgical and/or locoregional therapies Patients with progressive disease after surgical and/or locoregional therapies are eligible.
No prior systemic treatment (including systemic investigational agents) for HOC Prior treatment with herbal therapies, including traditional Chinese medicines, with anti-cancer activity noted in the label are allowed, provided that these medications are discontinued prior to randomization.
Life expectancy ≥3 months.

The efficacy and safety objectives and endpoints are described in the following tables. It is expected that the patient treated with the triple combination of Atezo+Bev+Tira herein will achieve one or more of the efficacy endpoints (ORR, PiS, OS, DOR and/or disease control) compared with placebo Atezo+Bev (i.e., without Tira), while having acceptable toxicity.

TABLE 44
Objectives and Corresponding Endpoints

| Primary Efficacy Objective | Corresponding Endpoint |
|---|---|
| To evaluate the efficacy of | ORR, defined as the proportion of |
| immunotherapy-based treatment combinations | patients with a complete response or partial |
| | response on two consecutive occasions 4 weeks |
| | apart, as determined by the investigator |
| | according to RECIST v1.1 |
| Secondary Efficacy Objective | Corresponding Endpoints |
| To evaluate the efficacy of | PFS after randomization, defined as the |
| immunotherapy-based treatment combinations | time from randomization to the first occurrence |
| | of disease progression or death from any cause |
| | (whichever occurs first), as determined by the |
| | investigator according to RECIST v1.1 |
| | OS after randomization, defined as the |
| | time from randomization to death from any |
| | cause |
| | OS at specific timepoints (e.g., 6 months) |
| | DOR, defined as the time from the first |
| | occurrence of a documented objective response |
| | to disease progression or death from any cause |
| | (whichever occurs first), as determined by the |
| | investigator according to RECIST v1.1 |
| | Disease control, defined as stable |
| | disease for 12 weeks or a complete or partial |
| | response, as determined by the investigator |
| | according to RECIST |
| Exploratory Efficacy Objective | Corresponding Endpoints |
| To evaluate the efficacy of immunotherapy- | ORR, PFS, DOR, and disease control as |
| based treatment combinations | determined by the investigator according to |
| | iRECIST and HCC mRECIST |

ADA = anti-drug antibody;
DOR = duration of response;
HCC = hepatocellular carcinoma;
HCC mRECIST = HCC-specific modified RECIST;
iRECIST = modified RECIST v1.1 for immune-based therapeutics;
NCI CTCAE v5.0 = National Cancer Institute Common Terminology Criteria for Adverse Events, Version 5.0;
ORR = objective response rate;
OS = overall survival;
PFS = progression-free survival;
PK = pharmacokinetic;
RECIST = Response Evaluation Criteria in Solid Tumors.

TABLE 45
Objectives and Corresponding Endpoints

| Safety Objective | Corresponding Endpoints |
|---|---|
| To evaluate the safety of | Incidence, nature, and severity of adverse events and |
| immunotherapy-based treatment | laboratory abnormalities, with severity determined |
| combinations | according to NCI CTCAE v5.0 |
| | Change from baseline in vital signs and ECG |
| | parameters |
| | Change from baseline in targeted clinical laboratory |
| | test results |
| Exploratory | |
| Pharmacokinetic Objectives | Corresponding Endpoints |
| To characterize the PK profile of | Plasma or serum concentration of each drug (as |
| drugs that are administered as part of | appropriate) at specified timepoints |
| an immunotherapy-based | treatment |
| combination | |
| To evaluate potential relationships | Relationship between plasma or serum concentration |
| between drug exposure and the | or PK parameters for each drug (as appropriate on |
| efficacy and safety of | the basis of available data) and efficacy endpoints |
| immunotherapy-based treatment | Relationship between plasma or serum concentration |
| combinations | or PK parameters for each drug (as appropriate on |
| | the basis of available data) and safety endpoints |
| Exploratory | |
| Immunogenicity Objectives | Corresponding Endpoint |
| To evaluate the immune response to | For drugs for which ADA formation is measured: |
| drugs that are administered as part of | presence of ADAs during the study relative to the |
| an immunotherapy-based treatment | presence of ADAs at baseline |
| combination | |
| To evaluate potential effects of ADAs | For drugs for which ADA formation is measured: |
| | relationship between ADA status and efficacy, safety, |
| | or PK endpoints |

ADA = anti-drug antibody;
DOR = duration of response;
HCC = hepatocellular carcinoma;
HCC mRECIST = HCC-specific modified RECIST;
iRECIST = modified RECIST v1.1 for immune-based therapeutics;
NCI CTCAE v5.0 National Cancer Institute Common Terminology Criteria for Adverse Events, Version 5.0;
ORR = objective response rate;
OS = overall survival;
PFS = progression-free survival;
PK = pharmacokinetic;
RECIST = Response Evaluation Criteria in Solid Tumors.

### Example 7. A Phase III, Randomized, Double Blind, Placebo-Controlled Study of Atezolizumab Plus Carboplatin and Etoposide with or without Tiragolumab in Patients with Untreated Extensive-Stage Small Cell Lung Cancer

The present example describes a randomized, Phase III, global, multicenter, double-blinded, placebo-controlled study designed to evaluate whether the anti-tumor effects of atezolizumab plus chemotherapy, as measured by OS, PFS, ORR, and DOR, can be improved with the addition of the anti-TIGIT antibody tiragolumab to atezolizumab and chemotherapy in patients with ES-SCLC. Safety and efficacy of tiragolumab in combination with atezolizumab and CE are compared with treatment with placebo in combination with atezolizumab and CE in patients who have ES-SCLC and are chemotherapy naive for their extensive-stage disease.

A. Study Design

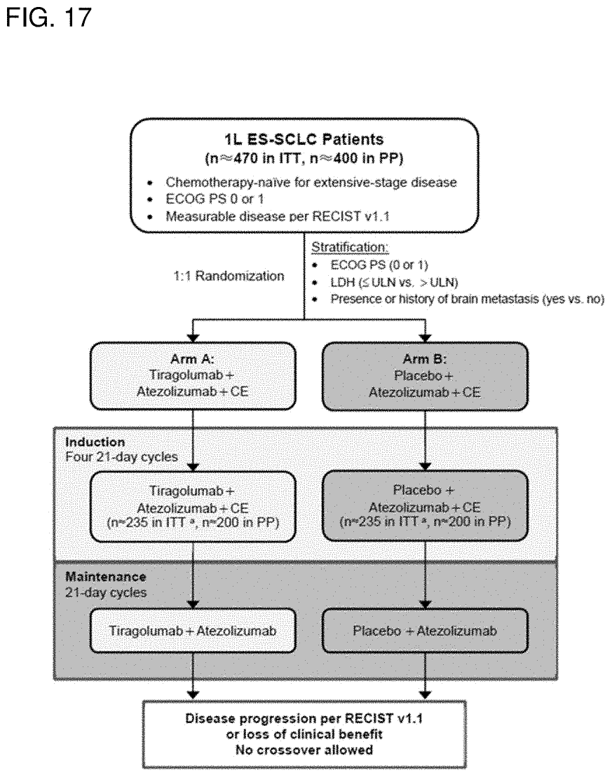

Described below are the details of a randomized, Phase III, multicenter, double-blinded, placebo-controlled study for evaluating the safety and efficacy of tiragolumab in combination with atezolizumab and CE compared with treatment with placebo in combination with atezolizumab and CE in patients who have ES-SCLC and are chemotherapy naïve for their extensive-stage disease. FIG. 17 illustrates the study design.

Eligible patients are stratified by Eastern Cooperative Oncology Group (ECOG) performance status (0 vs. 1), LDH (≤upper limit of normal (ULN) vs. >ULN), and presence or history of brain metastasis (yes vs. no) and randomized in a 1:1 ratio to receive one of the following treatment regimens as shown in Table 46. Further details regarding ECOG performance status are provided in Oken et al., Am. J. Clin Oncol. 1982, 5: 649-655.

Approximately 470 patients in total are randomized in the study. The primary population (PP) includes approximately 400 patients, assuming a 15% prevalence of presence or history of brain metastases at baseline. The intent-to-treat (ITT) population includes all patients.

TABLE 46
Study Treatment Arms

| | Induction Phase | Maintenance Phase |
|---|---|---|
| Treatment Arm | (Four 21-Day Cycles) | (All 21-Day Cycles after Cycle 4) |
| A | Tiragolumab + Atezolizumab + | Tiragolumab + Atezolizumab |
| | Carboplatin + Etoposide | |
| B | Tiragolumab + Placebo + Carboplatin + | Placebo + Atezolizumab |
| | Etoposide | |

Induction treatment is administered on a 21-day cycle for four cycles. Following the induction phase, patients continue maintenance therapy with either atezolizumab plus tiragolumab (Arm A) or atezolizumab plus placebo (Arm B). During the maintenance phase, prophylactic cranial irradiation (PCI) is permitted per local standard-of-care and is reported on the Prophylactic Cranial Irradiation electronic Case Report Form (eCRF). Palliative radiation for symptomatic management is allowed. The dosing and administration schedule for the treatment regimens in Table 46 are provided in Table 47 below.

TABLE 47
Dosing and Administration Schedule for Treatment Regimens

| | | | | Maintenance |
|---|---|---|---|---|
| Drugs (listed | Induction Phase | | | Phase |
| in order of | Cycles 1-4 | | | >Cycle 4 |
| administration) | Day 1 | Day 2 | Day 3 | Day 1 |
| Atezolizumab | 1200 mg | | | 1200 mg |
| (Arm A) or | | | | |
| placebo | | | | |
| (Arm B) | | | | |
| Tiragolumab | 600 mg | | | 600 mg |
| (Arm A) or | | | | |
| placebo | | | | |
| (Arm B) | | | | |
| Carboplatin | AUC = 5 | | | |
| | mg/ml/min | | | |
| etoposide | 100 mg/m² | 100 mg/m² | 100 mg/m² | |

On Day 1 of each cycle, all eligible patients are administered study drug infusions in the following order:
Arm A: atezolizumab, tiragolumab, carboplatin, and etoposide
Arm B: atezolizumab, placebo, carboplatin, and etoposide

Patients can receive anti-emetics and IV hydration for carboplatin and etoposide according to the local standard-of-care and manufacturer's instruction. Premedication with corticosteroids are minimized to the extent that is clinically feasible. All medications are recorded on the appropriate Concomitant Medications eCRF.

During the induction phase, study treatment should be administered in the following manner on Day 1:
1. Atezolizumab 1200 mg administered intravenously over 60 (±15) minutes (for the first infusion and shortening to 30 [±10] minutes for subsequent infusions) (see Table 48), followed by
2. Tiragolumab/placebo 600 mg administered intravenously over 60 (±15) minutes (for the first infusion and shortening to 30 [±10] minutes for subsequent infusions) (see Table 48), followed by
3. Carboplatin administered intravenously over 30-60 minutes to achieve an initial target area under the concentration-time curve (AUC) of 5 mg/mL/min (Calvert formula dosing), followed by
4. Etoposide (100 mg/m²) administered intravenously over 60 minutes During the induction phase, etoposide (100 mg/m²) will be also administered intravenously over 60 minutes on Days 2 and 3.

Cycles in which no chemotherapy is given do not count toward the total number of induction chemotherapy cycles. After the induction phase, patients begin maintenance therapy with atezolizumab plus tiragolumab/placebo. Suggested infusion times for carboplatin and etoposide are adapted in accordance with local standard-of-care. Administration of atezolizumab and tiragolumab/placebo are performed in a monitored setting where there is immediate access to trained personnel and adequate equipment and medicine to manage potentially serious reactions.

Treatment is continued until radiographic disease progression according to RECIST v1.1, or as long as patients are experiencing clinical benefit, as assessed by the investigator, in the absence of unacceptable toxicity or symptomatic deterioration attributed to disease progression after an integrated assessment of radiographic data, biopsy results (if available), and clinical status. Patients who meet the criteria for disease progression per RECIST v1.1 are permitted to continue study treatment (tiragolumab plus atezolizumab or placebo plus atezolizumab) if they meet all of the inclusion criteria.

### Atezolizumab

Patients receive 1200 mg atezolizumab administered by IV infusion on Day 1 of each 21-day cycle. The atezolizumab dose is fixed and is not dependent on body weight. Atezolizumab infusions are administered per the instructions outlined in Table 48. Atezolizumab dose is not modified. Further details on dose preparation, storage, and administration instructions for atezolizumab can be found in the pharmacy manual and/or the Atezolizumab Investigator's Brochure.

### Tiragolumab/Placebo

Following the administration of atezolizumab and an observation period (see Table 48), patients receive 600 mg tiragolumab/placebo administered by IV infusion on Day 1 of each 21-day cycle. The tiragolumab/placebo dose is fixed and is not dependent on body weight. Tiragolumab/placebo infusions are administered per the instructions outlined in Table 48. Tiragolumab/placebo dose is not modified. Further details on dose preparation, storage, and administration instructions for tiragolumab/placebo can be found in the pharmacy manual and/or the Tiragolumab Investigator's Brochure.

TABLE 48
Administration of First and Subsequent Atezolizumab and Tiragolumab/Placebo Infusions

| | First infusion | Subsequent infusion |
|---|---|---|
| Atezolizumab | No premedication is administered for | If the patient experienced an IRR |
| infusion | the first infusion of atezolizumab. | during any previous infusion of |
| | Vital signs (pulse rate, respiratory | atezolizumab, premedication with an |
| | rate, blood pressure, and | antihistamine and/or antipyretic may |
| | temperature) are recorded within 60 | be administered for subsequent |
| | minutes prior to starting the infusion | infusions at the discretion of the |
| | of atezolizumab. | investigator. |
| | Atezolizumab is infused over 60 | Vital signs (pulse rate, respiratory |
| | (±15) minutes. | rate, blood pressure, and |
| | If clinically indicated, vital signs are | temperature) are recorded within 60 |
| | measured every 15 (±5) minutes | minutes before starting the infusion of |
| | during the infusion and at 30 (±10) | atezolizumab. |
| | minutes after the infusion. | If the patient tolerated the first |
| | | infusion of atezolizumab well without |
| | | an IRR, the next infusion of |
| | | atezolizumab may be infused over 30 |
| | | (±10) minutes. |
| | | If no reaction occurs, subsequent |
| | | infusions of atezolizumab are |
| | | continued over 30 (±10) minutes. |
| | | Vital signs continue to be recorded |
| | | within 60 minutes prior to the infusion |
| | | Vital signs are recorded during the |
| | | infusion if clinically indicated. |
| Observation period | After the infusion of atezolizumab, | If the patient tolerated the previous |
| after infusion of | the patient begins a 60-minute | infusion of atezolizumab well without |
| atezolizumab | observation period. | an IRR, the observation period after |
| | Vital signs (pulse rate, respiratory | the next and following infusions may |
| | rate, blood pressure, and | be reduced to 30 minutes. |
| | temperature) are recorded at 30 | If the patient experienced an IRR in |
| | (±10) minutes after the infusion of | the previous infusion, the observation |
| | atezolizumab. | period should be 60 minutes. |
| | Patients are informed about | the If clinically indicated, vital signs |
| | possibility of delayed post-infusion | (pulse rate, respiratory rate, blood |
| | symptoms and instructed to contact | pressure, and temperature) are |
| | their study physician if they develop | recorded at 30 (±10) minutes after |
| | such symptoms. | the infusion of atezolizumab. |
| Tiragolumab/placebo | No premedication is allowed for the | If the patient experienced an IRR |
| infusion | first infusion of tiragolumab/placebo. | during any previous infusion of |
| | Vital signs (pulse rate, respiratory | tiragolumab/placebo, premedication |
| | rate, blood pressure, and | with an antihistamine and/or |
| | temperature) are recorded within 60 | antipyretic may be administered for |
| | minutes prior to starting the infusion, | subsequent infusions at the |
| | Tiragolumab/placebo is infused | discretion of the investigator. |
| | over 60 (±15) minutes. | Vital signs (pulse rate, respiratory |
| | Vital signs (pulse rate, respiratory | rate, blood pressure, and |
| | rate, blood pressure, and | temperature) are recorded within 60 |
| | temperature) are recorded every 15 | minutes before starting the infusion of |
| | (±5) minutes during the infusion. | tiragolumab/placebo. |
| | | If the patient tolerated the first or a |
| | | subsequent infusion of |
| | | tiragolumab/placebo well without an |
| | | IRR, the next infusion of |
| | | tiragolumab/placebo may be infused |
| | | over 30 (±10) minutes. |
| | | If no reaction occurs, subsequent |
| | | infusions of tiragolumab/placebo |
| | | continue over 30 (±10) minutes. |
| | | If the patient experienced an IRR |
| | | with the previous infusion, the next |
| | | infusion of tiragolumab/placebo is |
| | | administered over 60 (±15) minutes. |
| | | Vital signs continue to be recorded |
| | | within 60 minutes before starting |
| | | infusion of tiragolumab/placebo. |
| | | Vital signs are recorded during and |
| | | after the infusion if clinically |
| | | indicated. |
| Observation period | After the infusion of | If the patient tolerated the previous |
| after infusion of | tiragolumab/placebo, the patient | infusion of tiragolumab/placebo well |
| tiragolumab/placebo | begins a 60-minute observation | without an IRR, the observation |
| | period. | period is reduced to 30 minutes for |
| | Vital signs (pulse rate, respiratory | subsequent infusions. |
| | rate, blood pressure, and | If clinically indicated, vital signs |
| | temperature) are recorded at 30 | (pulse rate, respiratory rate, blood |
| | (±10) minutes after the infusion of | pressure, and temperature) are |
| | tiragolumab/placebo. | recorded at 30 (±10) minutes after |
| | Patients are informed about the | the infusion of tiragolumab/placebo. |
| | possibility of delayed post-infusion | Patients are informed about the |
| | symptoms and are instructed to | possibility of delayed post-infusion |
| | contact their study physician if they | symptoms and will be instructed to |
| | develop such symptoms. | contact their study physician if they |
| | | develop such symptoms. |

Carboplatin

During the induction phase, carboplatin is administered after completion of tiragolumab/placebo by IV infusion over 30-60 minutes to achieve an initial target AUC of 5 mg/mL/min (Calvert formula dosing) with standard anti-emetics per local practice guidelines. Premedication with corticosteroids may be minimized to the extent that is clinically feasible. Carboplatin infusion times are adapted in accordance with local standard-of-care.

The carboplatin dose of AUC 5 is calculated using the Calvert formula (Calvert et al., J. Clin. Oncol. 1989, 7:1748-56):

Calvert Formula:

Total dose (mg)=(target AUC)×(glomerular filtration rate [GFR]+25)
NOTE: The GFR used in the Calvert formula to calculate AUC-based dosing should not exceed 125 mL/min.

As used herein, the GFR is considered to be equivalent to the creatinine clearance (CRCL). The CRCL is calculated by institutional guidelines or by the method of Cockcroft and Gault, Nephron 1976, 16:31-41, using the following formula:

CRCL=(140−age)×(wt)

(×0.85 if female)

72×Scr
Where: CRCL=creatinine clearance in mL/min
   age=patient's age in years
   wt=patient's weight in kg
   Scr=serum creatinine in mg/dL
NOTE: For patients with an abnormally low serum creatinine level, GFR is estimated through use of a minimum creatinine level of 0.8 mg/dL or the estimated GFR is capped at 125 mL/min.

If a patient's GFR is estimated based on serum creatinine measurements by the isotope dilution mass spectroscopy method, physicians consider capping the dose of carboplatin for desired exposure (AUC) to avoid potential toxicity due to overdosing. On the basis of the Calvert formula described in the carboplatin label, the maximum doses can be calculated as follows:

Maximum carboplatin dose (mg)=target AUC (mg×min/mL)×(GFR+25 mL/min)

The maximum dose is based on a GFR estimate that is capped at 125 mL/min for patients with normal renal function. No higher estimated GFR values should be used.

For a target AUC=5, the maximum dose is 5×(125+25)=5×150=750 mg.

For a target AUC=4, the maximum dose is 4×(125+25)=4×150=600 mg.

### Etoposide

During the induction phase, on Day 1 of each cycle, etoposide (100 mg/m²) is administered by IV infusion over 60 minutes following carboplatin administration. On Days 2 and 3 of each cycle, etoposide (100 mg/m²) is administered by IV infusion over 60 minutes. Premedication is administered according to local standard-of-care. Premedication with corticosteroids may be minimized to the extent that is clinically feasible. Etoposide infusion times may be adapted in accordance with local standard-of-care.

Rationale for Evaluation of Patients without Brain Metastases at Baseline (Primary Population for Statistical Analysis)

The brain is a common site of metastases for ES-SCLC patients, with some studies showing as high as 18% of patients having brain metastases at diagnosis, and up to 80% are expected to have brain disease involvement during the first 2 years following diagnosis (Seute et al., Cancer, 100: 801-806, 2004; Pacheco and Bunn, Clin Lung Cancer, 20: 148-160, 2019). In general, benefits from the immunotherapy and chemotherapy combination have been shown in patients with ES-SCLC. However, this benefit may be attenuated in those with brain metastasis at baseline as seen in the IMpower133 trial where this patient subgroup had a hazard ratio (HR) of 1.07 compared to a HR of 0.68 for patients without brain metastases (Horn et al., New Engl J Med, 379: 2220-2229, 2018).

KEYNOTE-604 is another phase 3 trial that suggests that ES-SCLC patients with brain metastasis at diagnosis may achieve less or no overall survival benefit to first-line immunotherapy plus chemotherapy in comparison to those without. In fact, patients with brain metastases had an OS HR of 1.32 versus 0.75 for patients without brain metastases (Rudin et al., J Clin Oncology, 38: 2369-2379, 2020). The CASPIAN study initially reported an OS HR of 0.69 in patients with brain metastasis, however, in the updated analysis the HR became 0.79. The OS HR in those without brain metastasis in initial and updated reports was 0.74 and 0.76, respectively (Paz-Ares et al., N Engl J Med, 379: 2040-2051, 2018; Paz-Ares et al., Am Soc Clin Oncol 2020, abstract 9002, 2020).

It is critical to understand the benefit of the study regimen in both of the patient subgroups, thus, the testing statistical testing hierarchy was implemented to test the PP first, and then subsequently the ITT.

B. Assessments

Patients undergo tumor assessments at baseline and every 6 weeks (±7 days) for 48 weeks following Cycle 1, Day 1. A pretreatment tumor tissue (archival or freshly obtained) sample is submitted before or within four weeks after randomization. This specimen is accompanied by the associated pathology report. Although any available tumor tissue sample can be submitted, it is encouraged that representative tumor specimens in paraffin blocks (preferred) or 10 (or more) serial, freshly cut, unstained slides be submitted for biomarker analysis (e.g., PD-L1 status, markers related to immune- or SCLC-biology such as T-cell markers or non-inherited biomarkers identified through NGS on extracted DNA, RNA, or other biological molecules). Exemplary sample types include formalin-fixed paraffin-embedded (FFPE) samples prepared from resections, core needle, excisional, incisional, punch, forceps biopsies, fine-needle aspiration, and cell pellet specimens (e.g., from pleural effusion and lavage samples). Tumor tissue should be of good quality based on total and viable tumor content. Tumor tissue from bone metastases that is subject to decalcification is not advisable.

After completion of the Week 48 tumor assessment, tumor assessments occur every 9 weeks (±7 days) thereafter. Patients treated beyond disease progression per RECIST v1.1 undergo tumor assessments until treatment is discontinued. Patients who discontinue treatment for reasons other than radiographic disease progression per RECIST v1.1 (e.g., toxicity, symptomatic deterioration) will continue scheduled tumor assessments every 6 weeks (±7 days) for 48 weeks following Cycle 1, Day 1 and then every 9 weeks (±7 days) thereafter, regardless of whether the patient starts a new anti-cancer therapy.

Serum samples are collected to monitor tiragolumab and atezolizumab pharmacokinetics. In selected patients, plasma samples are collected for chemotherapy pharmacokinetics. Safety assessments include the incidence, nature, and severity of adverse events, protocol-mandated vital signs, and laboratory abnormalities.

Tumor biopsy is performed at the time of radiographic progression. Exemplary sample types include FFPE samples prepared from resections, core needle, excisional, incisional, punch, forceps biopsies, fine-needle aspiration, and cell pellet specimens (e.g., from pleural effusion and lavage samples). DNA and/or RNA extraction from tissue may be performed.

C. Patient Reported Outcomes

Patent Reported Outcomes (PRO) data are collected to document the treatment benefit and more fully characterize the clinical profile of tiragolumab+atezolizumab. PRO data are collected using the following instruments: EORTC QLQ-C30, single-item EORTC IL46, select items from the PRO-CTCAE, and the EQ-5D-5L.

The EORTC QLQ-C30 is a validated and reliable self-report measure (Aaronson et al., J. Natl. Cancer Inst. 1993, 85:365-76; Fitzsimmons et al., Eur. J. Cancer 1999, 35:939-41) that consists of 30 questions that assess five aspects of patient functioning (physical, emotional, role, cognitive, and social), three symptom scales (fatigue, nausea and vomiting, pain), global health/quality of life, and six single items (dyspnea, insomnia, appetite loss, constipation, diarrhea, and financial difficulties). Scale scores can be obtained for the multi-item scales.

The EORTC QLQ-C30 module takes approximately 15 minutes to complete. In addition, the single-item EORTC IL46 is collected. This validated single-item question assesses overall side-effect impact.

The PRO-CTCAE is a validated item bank that is used to characterize presence, frequency, severity, and interference with daily function of 78 patient-reportable symptomatic treatment toxicities (Basch et al., J. Natl Cancer Inst. 2014, 106:1-11; Dueck et al., JAMA Oncol. 2015, 1:1051-52). The PROCTCAE contains 124 questions that are rated on either a 5-point Likert scale (frequency, severity and interference) or dichotomously (presence/absence). Included treatment toxicity terms can be subjective, with or without observable components (e.g., vomiting and nausea, respectively), or primarily observable with subjective components (e.g., rash). The standard PRO-CTCAE recall period is “the past 7 days.” A subset of three symptoms that were deemed most applicable to the current treatments were selected for this study.

The EQ-5D-5L is a generic, preference-based health utility measure with questions about mobility, self-care, usual activities, pain/discomfort, and anxiety/depression that is used to build a composite of the patient's health status. The EQ-5D-5L takes approximately 2 minutes to complete. The EQ-5D-5L will be utilized in this study for economic modeling.

Paper versions of the PRO instruments are self-administered during the treatment visits and interviewer administered by site personnel to the patient over the telephone during follow-up visits so that data can be collected without mandating patients' travel to the clinical site. PRO data are entered into the study database by the site personnel.

To ensure instrument validity and that data standards meet health authority requirements, questionnaires scheduled for administration during a clinic visit are completed in their entirety by the patient prior to receiving any information on disease status, prior to the performance of non-PRO assessments that could bias patients' answers, and prior to the administration of study treatment, unless otherwise specified.

The questionnaires (EORTC QLQ-C30, EORTC IL46, PRO-CTCAE (select items), and EQ-5D-5L) are completed during the induction phase at Cycle 1, Day 1 (baseline) prior to administration of study drug; then at every study treatment cycle prior to administration of study drug (i.e., on Cycle 2, Day 1; Cycle 3, Day 1; and Cycle 4, Day 1). During the maintenance phase, the questionnaires are completed at every other study treatment cycle prior to administration of study drug (i.e., on Cycle 5, Day 1; Cycle 7, Day 1; Cycle 9, Day 1; etc.), and at the study treatment discontinuation visit. During survival follow-up, the PROs are collected at the first survival follow-up at 3 months (±30 days) and the second survival follow-up at 6 months (±30 days). Patients who discontinue study treatment for any reason other than radiographic disease progression per RECIST v1.1 (e.g., toxicity, symptomatic deterioration) will complete the EORTC QLQ-C30, PRO-CTCAE (select items), and EQ-5D-5L at each tumor assessment visit until radiographic disease progression per RECIST v1.1, death, loss to follow-up, consent withdrawal, or study termination by the Sponsor, whichever occurs first. Patients whose native language is not available with the questionnaires are exempted from completing all PRO assessments.

Adverse events reports are not derived from PRO data.

D. Endpoints

Investigator-assessed PFS (as assessed by the inventor according to RECIST v1.1) and OS in the PP are the co-primary endpoints for this study. Objective Response Rate in the PP and ITT population, Duration of Response in the PP and ITT population, and PFS and OS in the ITT population are secondary endpoints.

### Progression-Free Survival

PFS is the time between the date of randomization and the date of first documented disease progression (as assessed by investigators according to RECIST v1.1) or death, whichever occurs first. Patients who have not experienced disease progression or have not died by the data cutoff date are censored at the time of the last tumor assessment. Patients with no post-baseline tumor assessment are censored at the date of randomization.

PFS as an endpoint can reflect tumor growth and can be assessed before the determination of a survival benefit; additionally, its determination is not generally confounded by subsequent therapies. Whether an improvement in PFS represents a direct clinical benefit or a surrogate for clinical benefit depends upon the magnitude of the effect and the benefit-risk of the new treatment compared with available therapies (U.S. Food and Drug Administration (2007) Guidance for Industry: Clinical Trial Endpoints for the Approval of Cancer Drugs and Biologics).

To ensure the validity of investigator-assessed PFS as the primary endpoint, a number of measures have been implemented: a substantial target magnitude of benefit and study assessments that will allow a robust evaluation of benefit-risk (conventional RECIST v1.1 criteria to define radiographic disease progression with fixed assessment intervals that are identical in both treatment arms, and a robust definition of PFS and prospectively-defined methods to assess, quantify, and analyze PFS, including sensitivity analyses).

The primary analysis of the co-primary endpoint of PFS occurs when approximately 300 PFS events (75% of 400 patients) have been observed in the PP. This provides 96% power to detect a target HR of 0.56 for PFS at a two-sided significance level of 0.001, based on the following: PFS curve following the exponential distributions; median PFS of 5.2 months in the placebo+atezolizumab+EC arm and 9.2 months in the tiragolumab+atezolizumab+EC arm (corresponding to a target HR of 0.56); dropout rate of 5% over 12 months for PFS; and no interim analysis for PFS.

An observed HR of 0.68 or better for PFS corresponds to a statistically significant difference between the treatment arms. That is, an HR of 0.68 is the minimally detectable difference for the analysis; this corresponds to an improvement of 2.4 months in median PFS from 5.2 months in the placebo+atezolizumab+CE arm to 7.6 months in the tiragolumab+atezolizumab+CE arm.

Kaplan-Meier methodology is used to estimate the median PFS for each treatment arm, and Kaplan-Meier curves are constructed to provide a visual description of the difference between treatment arms. The Brookmeyer-Crowley methodology will be used to construct the 95% CIs for the median PFS for each treatment arm (Brookmeyer and Crowley, Biometrics 1982, 38: 29-41).

The PFS rates at 6 months and at 1 year after randomization are estimated using Kaplan-Meier methodology for each treatment arm, along with 95% CIs calculated using the standard error derived from Greenwood's formula. The 95% CI for the difference in PFS rates between the two treatment arms is estimated using the normal approximation method.

### Overall Survival

OS is a co-primary endpoint in this study. OS is the time between the date of randomization and death from any cause. Improvement in OS is generally accepted as the best measure of clinical benefit for patients with advanced/unresectable or metastatic lung cancer.

PFS and OS are compared between treatment arms with use of the stratified log-rank test. The HR for PFS and OS are estimated using a stratified Cox proportional hazards model. The 95% CI for the HR is provided. The stratification factors are those used for randomization from the IxRS (i.e., ECOG performance status and lactate dehydrogenase (LDH)). Stratification factor(s) may be removed from the stratified analyses if there is risk of overstratification. Analyses based on stratification factors recorded on the electronic Case Report Form (eCRF) will also be provided if considerable discrepancy is observed between IxRS and eCRF records. Results from an unstratified analysis are also provided.

The final analysis of the co-primary endpoint of OS occurs when approximately 288 deaths (72% of 400) have been observed in the PP. This provides 85% power to detect a target HR of 0.70 for OS at a two-sided significance level of 0.049, based on the following: OS curve following the exponential distributions; median OS of 12.3 months in the placebo+atezolizumab+EC arm and 17.6 months in the tiragolumab+atezolizumab+EC arm (corresponding to a target HR of 0.70); dropout rate of 5% over 24 months for OS; and one planned interim analysis for OS at approximately 70% of the information fraction, with the interim boundary for statistical significance determined based on the Lan-DeMets approximation of the O'Brien-Fleming function.

An observed HR of 0.789 or better for OS in the PP corresponds to a statistically significant difference between the treatment arms. That is, an HR of 0.789 is the minimally detectable difference for the analysis; this corresponds to an improvement of 3.3 months in median OS from 12.3 months in the placebo+atezolizumab+CE arm to 15.6 months in the tiragolumab+atezolizumab+CE arm.

The timing of the interim analyses and the final analysis for OS are summarized in Table 49, below, with the additional assumption on accrual.

TABLE 49
Analysis timing for overall survival

| | | | PP | |
|---|---|---|---|---|
| | Analysis Timing | | No. of Events | |
| | Months | Percentage | (Event | Power, |
| Type of Analysis | from FPI | Information | Patient Ratio) | %^{a} |
| OS IA | 23 | 70% | 202 (51%) | 54 |
| OS final analysis | 37 | 100% | 288 (72%) | 85 |

FPI = first patient in;
IA = interim analysis;
PP = primary population;
OS = overall survival
^{a}Power is calculated using two-sided α of 0.049

The OS interim analysis is conducted at the time of the final PFS analysis. Kaplan-Meier methodology is used to estimate the median OS for each treatment arm, and Kaplan-Meier curves are constructed to provide a visual description of the difference between treatment arms. The Brookmeyer-Crowley methodology will be used to construct the 95% CIs for the median OS for each treatment arm (Brookmeyer and Crowley, Biometrics 1982, 38: 29-41).

The OS rates at 1 and 2 years after randomization are estimated using Kaplan-Meier methodology for each treatment arm, along with 95% CIs calculated using the standard error derived from Greenwood's formula. The 95% CI for the difference in OS rates between the two treatment arms is estimated using the normal approximation method.

### Secondary Efficacy Endpoints

### Progression Free Survival and Overall Survival in the Intent-to-Treat Population

If the difference in OS in the PP is statistically significant, PFS and OS are tested in the ITT population, following the same α-allocation ratio (1:49) and α-recycle strategy for the analysis of the co-primary efficacy endpoints described above. PFS and OS in the ITT population will be analyzed at the same time with the PP, using the same methods as described above except that all three stratification factors used for randomization from the IxRS (i.e., ECOG performance status, LDH, and presence or history of brain metastasis) are included in the stratified analyses for the ITT population. Stratification factor(s) may be removed from the stratified analyses if there is risk of overstratification.

### Confirmed Objective Response Rate

A confirmed objective response can either be a confirmed CR or a PR on 2 consecutive occasions ≥4 weeks apart, as determined by the investigator using RECIST v1.1. Patients not meeting these criteria, including patients without any post-baseline tumor assessment, are considered non-responders.

Confirmed ORR is defined as the proportion of patients who had a confirmed objective response. The analysis population for confirmed ORR is the PP and ITT population with measurable disease at baseline. An estimate of confirmed ORR and its 95% CI is calculated using the Clopper Pearson method for each treatment arm. Cis for the difference in confirmed ORRs between the two treatment arms are determined using the normal approximation to the binomial distribution.

### Duration of Response

DOR of confirmed response is assessed in patients who had a confirmed objective response as determined by the investigator using RECIST v1.1 in the PP and the ITT population. DOR of confirmed response is the time interval from the date of the first occurrence of a confirmed objective response until the first date that progressive disease as determined by the investigator according to RECIST v1.1 or death is documented, whichever occurs first. Patients who have not progressed and who have not died at the time of analysis will be censored at the time of last tumor assessment date. DOR is based on a non-randomized subset of patients (specifically, patients who achieved an objective response); therefore, formal hypothesis testing is not performed for this endpoint. Comparisons between treatment arms are made for descriptive purposes. The methodologies detailed for the PFS analysis is used for the DOR analysis.

### Stratification Factors

Stratification factors are those used for randomization from the IxRS (i.e., ECOG performance status, LDH, and presence or history of brain metastasis). If at least one stratum (i.e., a combination of stratification factor levels across ECOG performance status [0 vs 1], LDH [≤ULN vs. >ULN], and presence or history of brain metastasis [yes vs. no] per IxRS) has less than 10 events (PFS or OS events), the stratification factor (one of 3 stratification factors: ECOG performance status, LDH, and presence or history of brain metastasis per IxRS) that contains the level with the smallest number of patients is removed from the stratified analyses. The removal of the stratification factor continues until there is no stratum with less than 10 events (PFS or OS events). The final set of stratification factors used in stratified analyses is applied to all endpoints where stratified analyses are planned. Analyses based on stratification factors recorded on the electronic Case Report Form (eCRF) is also provided if considerable discrepancy is observed between IxRS and eCRF records.

E. Eligibility

Patients are eligible if they are chemotherapy-naive for their ES-SCLC and meet eligibility criteria, including the following: age 18 years or older; ability to comply with the study protocol, in the investigator's judgment; ECOG performance status of 0 or 1; histologically or cytologically confirmed ES-SCLC (per the modified Veterans Administration Lung Study Group (VALG) staging system (Micke et al., Lung Cancer 2002, 37: 271-6)); no prior systemic treatment for ES-SCLC; patients who have received prior chemoradiotherapy for limited-stage SCLC must have had treatment with curative intent and a treatment-free interval of at least 6 months between the last dose/cycle of chemotherapy, thoracic radiotherapy, or chemoradiotherapy and the diagnosis of ES-SCLC; measurable disease, as defined by RECIST v1.1 (previously irradiated lesions can only be considered as measurable disease if (1) disease progression has been unequivocally documented at that site since radiation therapy, and (2) the previously irradiated lesion is not the only site of measurable disease); submission of a pre-treatment tumor tissue sample; for patients receiving therapeutic anticoagulation: stable anticoagulant regimen; negative HIV test at screening; negative hepatitis B surface antigen (HBsAg) test at screening; adequate hematologic and end-organ function, defined by the following laboratory test results, obtained within 14 days prior to initiation of study treatment (Day 1 of Cycle 1): absolute neutrophil count (ANC) ≥1.5×10⁹/L (1500/μL) without granulocyte colony-stimulating factor support; lymphocyte count ≥0.5×10⁹/L (500/μL); platelet count ≥100×10⁹/L (100,000/μL) without transfusion; hemoglobin 90 g/L (9 g/dL) (patients may be transfused or receive erythropoietic treatment as per local standard of care to meet this criterion); INR or aPTT ≤1.5×ULN (for patients not receiving therapeutic anticoagulation, INR, or aPTT ≤1.5×ULN); AST, ALT, and ALP ≤2.5×ULN, with the following exceptions: (patients with documented liver metastases: AST and/or ALT ≤5×ULN; patients with documented liver or bone metastases: ALP ≤5× ULN); bilirubin ≤1.5×ULN (with the following exception: patients with known Gilbert disease: bilirubin ≤3×ULN); creatinine ≤1.5×ULN; albumin ≥25 g/L (2.5 g/dL).

Exclusion criteria include: symptomatic or actively progressing CNS metastases (note: asymptomatic patients with treated (i.e., local CNS-directed therapy) or untreated CNS lesions are eligible, provided that all of the following criteria are met: measurable disease, per RECIST v1.1, must be present outside the CNS; the patient has no history of intracranial hemorrhage or spinal cord hemorrhage from CNS disease; metastases are limited to the cerebellum or the supratentorial region (i.e., no metastases to the midbrain, pons, medulla, or spinal cord); the patient has no symptoms caused by CNS disease (i.e., no headache, nausea, vomiting, convulsion, paralysis, etc.); the patient has no ongoing requirement for anticonvulsants for CNS disease; the patient has no ongoing requirement for dexamethasone/corticosteroids for CNS disease (previously untreated patients must also not have any history of requiring or receiving dexamethasone/corticosteroids for CNS disease); for patients with previously treated CNS metastases, there is no evidence of interim CNS progression between the completion of CNS-directed therapy and randomization; for previously untreated patients, there is no evidence of brain edema related to CNS disease (e.g., vasogenic edema); for previously untreated patients, a brain magnetic resonance imaging (MRI) scan with contrast is required at screening and is the preferred modality for all subsequent scheduled follow-up tumor assessments (note: computed tomography (CT) scan with contrast may be acceptable for all subsequent scheduled follow-up tumor assessments if the following criteria are met: both brain MRI and CT scan with contrast must be performed at screening to assess untreated CNS disease; the CT scan with contrast can be used to reliably evaluate lesions identified on the screening MRI with contrast; if CT scan with contrast cannot be used to reliably evaluate lesions identified on the screening MRI with contrast, then MRI scan with contrast must be used at all subsequent scheduled follow-up tumor assessments; and it the same modality is used at every tumor assessment; spinal cord compression not definitively treated with surgery and/or radiation, or previously diagnosed and treated spinal cord compression without evidence that disease has been clinically stable for ≥1 week prior to randomization; leptomeningeal disease; uncontrolled pleural effusion, pericardial effusion, or ascites requiring recurrent drainage procedures (once monthly or more frequently); patients with indwelling catheters (e.g., PLEURX®) are allowed regardless of drainage frequency; uncontrolled or symptomatic hypercalcemia (ionized calcium >1.5 mmol/L, total serum calcium >12 mg/dL, or corrected calcium >ULN); known clinically significant liver disease, including active viral, alcoholic, or other hepatitis, cirrhosis, and inherited liver disease, or current alcohol abuse; malignancies other than SCLC within 5 years prior to randomization, with the exception of those with a negligible risk of metastasis or death (e.g., expected 5-year OS >90%) treated with expected curative outcome (such as adequately treated carcinoma in situ of the cervix, basal or squamous-cell skin cancer, localized prostate cancer treated surgically with curative intent, ductal breast carcinoma in situ treated surgically with curative intent); active or history of autoimmune disease or immune deficiency, with the following exceptions: (patients with a history of autoimmune-related hypothyroidism who are on thyroid-replacement hormone therapy are eligible for the study; patients with controlled Type 1 diabetes mellitus who are on an insulin regimen are eligible for the study; patients with eczema, psoriasis, lichen simplex chronicus, or vitiligo with dermatologic manifestations only (e.g., patients with psoriatic arthritis are excluded) are eligible for the study provided all of the following conditions are met: rash must cover <10% of body surface area; disease must be well controlled at baseline and require only low-potency topical corticosteroids; no occurrence of acute exacerbations of the underlying condition requiring psoralen plus ultraviolet A radiation, methotrexate, retinoids, biologic agents, oral calcineurin inhibitors, or high-potency or oral corticosteroids within the previous 12 months; history of idiopathic pulmonary fibrosis, organizing pneumonia (e.g., bronchiolitis obliterans), drug-induced pneumonitis, or idiopathic pneumonitis, or evidence of active pneumonitis on screening chest CT scan; history of radiation pneumonitis in the radiation field (fibrosis) is permitted; active EBV infection or known or suspected chronic active EBV infection at screening; patients positive for EBV IgG and/or EBV nuclear antigen (EBNA) are eligible only if EBV IgM and/or EBV polymerase chain reaction (PCR) are negative; active tuberculosis; severe infection at the time of randomization, including but not limited to hospitalization for complications of infection, bacteremia, or severe pneumonia; significant cardiovascular disease, such as New York Heart Association cardiac disease (Class II or greater), myocardial infarction, or cerebrovascular accident within 3 months prior to randomization, unstable arrhythmias, or unstable angina; patients with known coronary artery disease, congestive heart failure not meeting the above criteria, or left ventricular ejection fraction <50% must be on a stable medical regimen that is optimized in the opinion of the treating physician, in consultation with a cardiologist if appropriate; major surgical procedure other than for diagnosis within 28 days prior to randomization or anticipation of need for a major surgical procedure during the course of the study; prior allogeneic bone marrow transplantation or solid organ transplant; any other diseases, metabolic dysfunction, physical examination finding, or clinical laboratory finding giving reasonable suspicion of a disease or condition that contraindicates the use of an investigational drug or that may affect the interpretation of the results or render the patient at high risk for treatment complications; patients with illnesses or conditions that interfere with their capacity to understand, follow, and/or comply with study procedures; treatment with any other investigational agent within 28 days prior to initiation of study treatment; current treatment with anti-viral therapy for HBV or HCV; administration of a live, attenuated vaccine within 4 weeks before randomization or anticipation that such a live attenuated vaccine will be required during the study; patients must not receive live, attenuated influenza vaccines (e.g., FLUMIST®) within 4 weeks prior to randomization, during treatment, and for 5 months following the last dose of study treatment; prior treatment with CD137 agonists or immune checkpoint blockade therapies, anti-CTLA-4, anti-TIGIT, anti-PD-1, and anti-PD-L1 therapeutic antibodies; treatment with systemic immunostimulatory agents (including, but not limited to, interferon and interleukin 2 (IL-2)) within 4 weeks or 5 drug elimination half-lives (whichever is longer) prior to randomization; treatment with systemic immunosuppressive medications (including, but not limited to corticosteroids, cyclophosphamide, azathioprine, methotrexate, thalidomide, and anti-tumor necrosis factor (anti-TNF) agents) within 1 week prior to randomization or anticipation of need for systemic immunosuppressive medication during study treatment, with the following exceptions: patients who received acute, low-dose systemic immunosuppressant medication or a one-time pulse dose of systemic immunosuppressant medication (e.g., 48 hours of corticosteroids for a contrast allergy) are eligible for the study after Medical Monitor confirmation has been obtained; patients who received mineralocorticoids (e.g., fludrocortisone), corticosteroids for chronic obstructive pulmonary disease (COPD) or asthma, or low-dose mineralocorticoids for orthostatic hypotension or low-dose mineralocorticoids and corticosteroids for adrenal insufficiency are eligible for the study; history of severe allergic anaphylactic reactions to chimeric or humanized antibodies or fusion proteins; known hypersensitivity to Chinese hamster ovary cell products or to any component of the tiragolumab or atezolizumab formulations; history of allergic reactions to carboplatin or etoposide; pregnancy or breastfeeding, or intention of becoming pregnant during study treatment or within 90 days after the final dose of tiragolumab or placebo, 5 months after the final dose of atezolizumab, or for 6 months after the final dose of carboplatin or etoposide; women of childbearing potential must have a negative serum pregnancy test result within 14 days prior to initiation of study treatment.

F. Cytokine Release Syndrome

No premedication is indicated for the administration of Cycle 1 of tiragolumab/placebo or atezolizumab. However, patients who experience cytokine-release syndrome (CRS) with tiragolumab/placebo or atezolizumab may receive premedication with antihistamines, anti-pyretics, and/or analgesics (e.g., acetaminophen) for subsequent infusions. CRS is defined as a supraphysiologic response following administration of any immune therapy that results in activation or engagement of endogenous or infused T cells and/or other immune effector cells. Symptoms can be progressive, always include fever at the onset, and may include hypotension, capillary leak (hypoxia), and end-organ dysfunction (Lee et al., Biol Blood Marrow Transplant, 25(4): 625-638, 2019). CRS has been well documented with chimeric antigen receptor T-cell therapies and bispecific T-cell engager antibody therapies but has also been reported with immunotherapies that target PD-1 or PD-L1 (Rotz et al., Pediatr Blood Cancer, 64: e26642, 2017; Adashek and Feldman, J Oncol Practice, 15: 502-504, 2019), including atezolizumab.

### Example 8. A Phase II, Randomized, Double-Blind, Placebo-Controlled Study of Tiragolumab in Combination with Atezolizumab Plus Pemetrexed and Carboplatin/Cisplatin Versus Pembrolizumab Plus Pemetrexed and Carboplatin/Cisplatin in Patients with Previously Untreated Advanced Non-Squamous Non-Small Cell Lung Cancer

The present example describes a randomized, Phase II, global, multicenter, double-blind study designed to evaluate the efficacy and safety of tiragolumab in combination with atezolizumab plus pemetrexed and carboplatin/cisplatin compared with placebo in combination with pembrolizumab plus pemetrexed and carboplatin/cisplatin in patients with previously untreated, locally advanced unresectable or metastatic non-squamous non-small cell lung cancer (NSCLC).

A. Study Design

Eligible patients of this study include previously untreated male and female patients age ≥18 years with Eastern Cooperative Oncology Group (ECOG) Performance Status (0 vs. 1) who have locally advanced unresectable or metastatic non-squamous NSCLC, with no EGFRor ALKgenomic aberrations.

Patients whose tumors have a known EGFR or ALK rearrangement are excluded from the study. Patients with tumors with unknown EGFR or ALK mutational status are required to be tested prior to enrollment. Eligible patients are stratified by PD-L1 expression (tumor proportion score (TPS)/tumor cell (TC)<1% vs. 1%-49% vs. ≥50% by local or central assay), geographic region (Asia vs. Non-Asia), and ECOG Performance Status (0 vs. 1) and are randomized 1:1 to receive the following treatment regimens:

Arm A: tiragolumab in combination with atezolizumab plus pemetrexed and cisplatin/carboplatin;

Arm B: placebo in combination with pembrolizumab plus pemetrexed and cisplatin/carboplatin.

The selection of carboplatin or cisplatin is per investigator's choice. The choice of carboplatin or cisplatin is made prior to randomization and cannot be changed after Day 1 of Cycle 1.

The randomization scheme is designed to ensure that an approximately equal number of patients with an adequate representation of all PD-L1 expression subgroups (TPS/TC<1%, 1%-49%, and ≥50%) are enrolled. In order to reflect a natural distribution of PD-L1 expression observed in first-line NSCLC patients, the proportion of patients enrolled into each PD-L1 subgroup is capped at approximately 80 patients (approximately 40% of total planned enrollment) per central testing with investigational VENTANA PD-L1 (SP263) Companion Diagnostics (CDx) Assay. To account for differences in local and central results, selective enrollment based on local PD-L1 status is implemented if enrollment into a subgroup, as defined by central results, is on a trajectory to exceed the cap limits.

Induction treatment for Arm A and Arm B is administered on a 21-day cycle for 4 cycles. Following the induction phase, patients will continue maintenance therapy as outlined below.
Arm A: tiragolumab in combination with atezolizumab plus pemetrexed
Arm B: placebo in combination with pembrolizumab plus pemetrexed

Patients undergo tumor assessment at baseline and every 6 weeks (±7 days) for 48 weeks following Cycle 1, Day 1, regardless of treatment dose delays. After completion of Week 48 tumor assessment, tumor assessments are required every 9 weeks (±7 days) thereafter, regardless of dose delays, until radiographic disease progression per RECIST v1.1, withdrawal of consent, study termination, or death, whichever occurs first. Patients who are treated beyond disease progression per RECIST v1.1 will undergo tumor assessments at the frequency described above until study treatment is discontinued. Patients who discontinue treatment for reasons other than radiographic disease progression per RECIST v1.1 (e.g., toxicity, symptomatic deterioration) will continue scheduled tumor assessments at the same frequency as would have been followed if the patient had remained on study treatment (i.e., every 6 weeks [±7 days] for 48 weeks following Cycle 1, Day 1, and then every 9 weeks [±7 days] thereafter, until radiographic disease progression per RECIST v1.1, withdrawal of consent, study termination, or death, whichever occurs first), regardless of whether the patient starts a new anti-cancer therapy.

Patients are asked to complete patient-reported outcomes questionnaires (EORTC QLQ-C30, EORTC QLQ-LC13, and EORTC IL46) during treatment, at study treatment discontinuation, and at the study treatment discontinuation visit.

Safety assessments at study visits include the incidence, nature, and severity of adverse events, protocol-mandated vital signs, laboratory abnormalities, and other protocol-specified tests that are deemed critical to the safety evaluation of the study.

During the study, serum samples are collected to monitor tiragolumab and atezolizumab pharmacokinetics and to detect the presence of antibodies to tiragolumab and atezolizumab.

Patient samples, including archival and fresh tumor tissue, serum, plasma, and blood samples are collected for exploratory biomarker assessments.

During the study, patients who meet criteria for disease progression per RECIST v1.1 and show evidence of clinical benefit may continue treatment with tiragolumab/placebo and atezolizumab/pembrolizumab at the investigator's discretion, provided that the patients meet all of the following criteria:
Evidence of clinical benefit, as assessed by the investigator
Absence of symptoms and signs (including worsening of laboratory values (e.g., new or worsening hypercalcemia)) indicating unequivocal progression of disease
No decline in ECOG Performance Status that can be attributed to disease progression
Absence of tumor progression at critical anatomical sites (e.g., leptomeningeal disease) that cannot be managed by protocol-allowed medical interventions
Patients must provide written informed consent to acknowledge deferring other treatment options in favor of continuing study treatment at the time of initial radiographic progression per RECIST v1.1.

Investigator assessment of overall tumor response at all timepoints is based only on RECIST v1.1. Objective response per iRECIST is calculated programmatically on the basis of investigator assessments of individual lesions at each specified timepoint.

B. Dosing and Administration

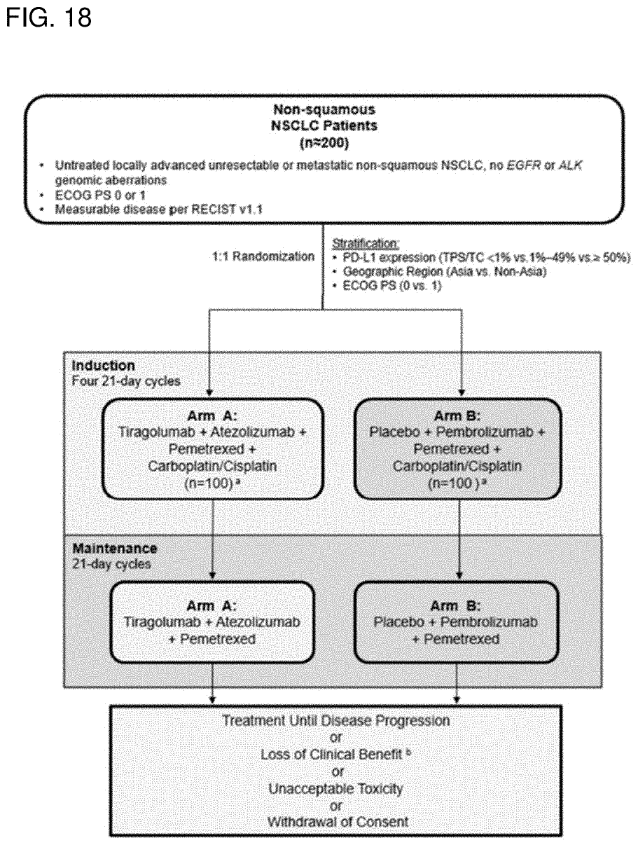

The treatment regimens are summarized in FIG. 18.

Atezolizumab and Pembrolizumab

Patients receive fixed-dose 20 mL (1200 mg) atezolizumab in Arm A or 8 mL (200 mg) pembrolizumab in Arm B on Day 1 of each 21-day cycle. Atezolizumab/pembrolizumab infusions are administered per the instructions outlined in Table 50.

TABLE 50
Administration of First and Subsequent Infusions of Atezolizumab, Pembrolizumab, Tiragolumab, and Placebo

| | First Infusion | Subsequent Infusions |
|---|---|---|
| Atezolizumab/pembrolizumab | No premedication is permitted | If the patient experienced an |
| infusion | for the first infusion of | IRR with any previous infusion of |
| | atezolizumab/pembrolizumab. | atezolizumab/pembrolizumab, |
| | Vital signs (pulse rate, | premedication with an |
| | respiratory rate, blood pressure, | antihistamine and/or antipyretic |
| | and temperature) are recorded | may be administered for |
| | within 60 minutes prior to starting | subsequent doses and beyond at |
| | the infusion. | the discretion of the investigator. |
| | Atezolizumab/pembrolizumab | Vital signs are recorded within |
| | are infused over 60 (±15) | 60 minutes prior to the infusion. |
| | minutes. | Atezolizumab/pembrolizumab is |
| | If clinically indicated, vital signs | infused over 30 (±10) minutes if |
| | are recorded every 15 (±5) | the previous infusion was |
| | minutes during the infusion. | tolerated without an IRR or 60 |
| | | (±15) minutes if the patient |
| | | experienced an IRR with the |
| | | previous infusion. |
| | | If the patient experienced an |
| | | IRR with the previous infusion, or |
| | | if clinically indicated, vital signs |
| | | are recorded during the infusion. |
| Observation period after | After the infusion of | If the patient tolerated the |
| atezolizumab/pembrolizumab | atezolizumab/pembrolizumab, the | previous |
| infusion | patient begins a 60-minute | atezolizumab/pembrolizumab |
| | observation period. | infusion well without infusion- |
| | Vital signs are recorded at 30 | associated adverse events, the |
| | (±10) minutes after the infusion | observation period after the next |
| | of atezolizumab/pembrolizumab. | and following infusions may be |
| | Patients are informed about the | reduced to 30 minutes. |
| | possibility of delayed post- | If the patient experienced |
| | infusion symptoms and instructed | infusion-associated adverse |
| | to contact their study physician if | events in the previous infusion, |
| | they develop such symptoms. | the observation period is 60 |
| | | minutes. |
| | | If clinically indicated, vital signs |
| | | are recorded at 30 (±10) minutes |
| | | after the infusion of |
| | | atezolizumab/pembrolizumab. |
| Tiragolumab/placebo infusion | No premedication is permitted | If the patient experienced an |
| | prior to tiragolumab/placebo | IRR during any previous infusion |
| | infusion. | of tiragolumab/placebo, |
| | Vital signs (pulse rate, | premedication with an |
| | respiratory rate, blood pressure, | antihistamine and/or anti-pyretic |
| | and temperature) are recorded | may be administered for |
| | within 60 minutes prior to the | subsequent doses at the |
| | infusion. | discretion of the investigator. |
| | Tiragolumab/placebo is infused | Vital signs are recorded within |
| | over 60 (±15) minutes. | 60 minutes prior to the |
| | Vital signs are recorded every | tiragolumab/placebo infusion. |
| | 15 (±5) minutes during the | Tiragolumab/placebo is |
| | infusion. | infused over 30 (±10) minutes |
| | | if the previous infusion was |
| | | tolerated without an infusion- |
| | | related reaction, or 60 (±15) |
| | | minutes if the patient experienced |
| | | an infusion-related reaction with |
| | | the previous infusion. |
| | | Vital signs are recorded |
| | | during the infusion if clinically |
| | | indicated. |
| Observation period after | After the infusion of | If the patient tolerated the |
| tiragolumab/placebo infusion | tiragolumab/placebo, the patient | previous infusion of |
| | begins a 60-minute observation | tiragolumab/placebo well without |
| | period. | infusion-associated adverse |
| | Vital signs are recorded at 30 | events, the observation period |
| | (±10) minutes after the infusion | may be reduced to 30 minutes. |
| | of tiragolumab/placebo. | If the patient experienced an |
| | Patients are informed about the | infusion-associated adverse event |
| | possibility of delayed post- | in the previous infusion, the |
| | infusion symptoms and are | observation period is 60 minutes. |
| | instructed to contact their study | If clinically indicated, vital signs |
| | physician if they develop such | are recorded at 30 (±10) minutes |
| | symptoms. | after the infusion of |
| | | tiragolumab/placebo. |
| | | Patients are informed about the |
| | | possibility of delayed post-infusion |
| | | symptoms and are instructed to |
| | | contact their study physician if |
| | | they develop such symptoms. |

Tiragolumab and Placebo

Patients receive fixed-dose 10 mL (600 mg) tiragolumab in Arm A or 10 mL placebo in Arm B on Day 1 of each 21-day cycle. Tiragolumab/placebo infusions are administered per the instructions outlined in Table 50.

Pemetrexed, Cisplatin, and Carboplatin

Table 51 lists the doses and suggested infusion times for treatment administration for pemetrexed, carboplatin or cisplatin.

TABLE 51
Treatment Regimen for Pemetrexed, Carboplatin or Cisplatin

| Study | Dose | Induction Period | Maintenance |
|---|---|---|---|
| Drug | and Route | (Four Cycles) | (Until PD) |
| Pemetrexed | 500 mg/m2 IV | Over approximately | Over approximately |
| | | 10 minutes on | 10 minutes on |
| | | Day 1 Q3W | Day 1 Q3W |
| Carboplatin | AUC 5 IV | Over approximately | Not applicable |
| | | 30-60 minutes on | |
| | | Day 1 Q3W | |
| Cisplatin | 75 mg/m2 IV | Over 1-2 hours | Not applicable |
| | | on Day 1 Q3W | |

AUC = area under the concentration-time curve;
IV = intravenous;
PD = progressive disease;
Q3W = every 3 weeks.

Patients receive anti-emetics and IV hydration for platinum-pemetrexed treatments according to the local standard of care and manufacturer's instruction. However, due to their immunomodulatory effects, premedication with steroids is limited when clinically feasible. In addition, in the event of pemetrexed-related skin rash, topical steroid use is recommended as front-line treatment whenever clinically feasible.

Table 52 lists the suggested premedication for pemetrexed.

TABLE 52
Premedication for Pemetrexed

| Premedication | Dose and Route | Timing |
|---|---|---|
| Dexamethasone | 4 mg PO | Twice daily the day before, |
| | | the day of, and the day after |
| | | pemetrexed administration |
| Folic acid | 350-1000 μg PO | Once daily beginning at least |
| | | 5-7 days before Cycle 1, Day 1 |
| | | and continuing until 3 weeks after |
| | | discontinuation of pemetrexed |
| Vitamin B₁₂ | 1000 μg IM | Q9W beginning Cycle 1, Day 1 |
| | | and continuing until 3 weeks after |
| | | discontinuation of pemetrexed |

IM = intramuscular;
PO = oral;
Q9W = every 9 weeks.

During the induction phase, a chemotherapy cycle counts toward the four cycles of induction chemotherapy as long as at least one platinum-based chemotherapy component has been administered at least once during a 21-day cycle. Cycles in which no chemotherapy is given do not count toward the total number of induction chemotherapy cycles.

Dose Modifications

Dose modifications for pemetrexed and cisplatin/carboplatin are permitted for toxicity according to the prescribing information and local standard-of-care.

Dose modification guidelines are provided below. Once reduced, the dose cannot be increased back to 100%.

Treatment with pemetrexed or cisplatin/carboplatin is discontinued if a patient experiences any hematologic or non-hematologic Grade 3 or Grade 4 toxicity after two dose reductions or treatment is delayed for more than 63 days due to toxicities.

Hematologic Toxicity

At the start of each cycle, the ANC is ≥1500/μL and the platelet count is 100,000/μL. Treatment could be delayed for up to 63 days to allow sufficient time for recovery. Growth factors may be used in accordance with American Society of Clinical Oncology (ASCO) and NCCN guidelines (Smith et al. 2015; NCCN 2019). Upon recovery, dose adjustments at the start of a subsequent cycle are made on the basis of the lowest platelet and neutrophil values from the previous cycle (Table 53). In the event that dose adjustments are needed for both ANC and platelets, patients are to receive the lower dose.

TABLE 53
Chemotherapy Dose Modification for Hematologic Toxicities

| Toxicity^{a} | Dose |
|---|---|
| ANC <500/μL and platelets ≥50,000/μL | 75% of previous dose |
| Platelets <25,000/μL, regardless of ANC | 75% of previous dose |
| Platelets <50,000/μL with Grade | 50% of previous dose |
| 2 bleeding, regardless of ANC | |
| ANC <1000/μL plus fever of ≥38.5° C. | 75% of previous dose |

^{a}Nadir of prior cycle.

Investigators are vigilant and alert to early and overt signs of myelosuppression, infection, or febrile neutropenia so that these complications can be promptly and appropriately managed. Patients are made aware of these signs and encouraged to seek medical attention at the earliest opportunity.

If chemotherapy is withheld because of hematologic toxicity, full blood counts (including differential WBC) are obtained weekly until the counts reach the lower limits for treatment as outlined. The treatment is then be resumed.

No dose reductions are recommended for anemia. Patients are supported per the investigator's institution's guidelines.

Non-Hematologic Toxicity

For a non-hematologic toxicity (Table 54), treatment is delayed for up to 63 days until resolution to less than or equal to the patient's baseline value (or Grade ≤1 if the patient did not have that toxicity at baseline). Dose reductions at the start of the subsequent cycle are made on the basis of non-hematologic toxicities from the dose administered in the preceding cycle. Table 54 provides recommended dose modifications for non-hematologic toxicities.

TABLE 54
Chemotherapy Dose Modification for Hematologic Toxicities

| | | Adjusted Dose as % |
|---|---|---|
| Toxicity | | of Previous Dose^{a} |
| Diarrhea | Grade 3 or 4^{b} | 75% |
| Nausea/vomiting | Grade 3 or 4^{c} | 75% |
| Neurotoxicity | Grade 2 | 75% |
| | Grade 3 or 4 | 50% or permanent |
| | | discontinuation |
| Transaminase | Grade 3 | 75% |
| elevation | Grade 4 | Discontinue |
| Other | Grade 3 or 4 | 75% |

AUC = area under the concentration-time curve.
^{a}If deemed appropriate by the investigator, adjust carboplatin dose to the specified percentage of the previous AUC.
^{b}Grade 3 or 4 diarrhea that occurs on adequate anti-diarrhea medication or any grade of diarrhea requiring hospitalization.
^{c}Despite the use of anti-emetics.

Diarrhea is controlled with adequate anti-diarrhea medication. Nausea and/or vomiting may be controlled with adequate anti-emetics. For Grade 3 or 4 neurotoxicity chemotherapy is resumed at 50% of the previous dose upon improvement or discontinued immediately (based on investigator's clinical judgment).

C. Concomitant Therapy

Concomitant therapy consists of any medication (e.g., prescription drugs, over-the-counter drugs, vaccines, herbal or homeopathic remedies, nutritional supplements) used by a patient in addition to protocol-mandated treatment from 7 days prior to initiation of study drug to the study discontinuation visit. All such medications are reported to the investigator and recorded.

Permitted Therapy

Patients are permitted to use the following therapies during the study:
Oral contraceptives with a failure rate of <1% per year
Hormone-replacement therapy
Palliative radiotherapy (e.g., treatment of known bony metastases or symptomatic relief of pain) as outlined below:
   In patients without documentation of radiographic disease progression, it is strongly encouraged to maximize supportive care for symptomatic management and to avoid radiotherapy that will interfere with the assessment of tumor target lesions. Treatment with tiragolumab/placebo and atezolizumab/pembrolizumab may be continued during palliative radiotherapy.
Receptor activator of nuclear factor-κB ligand-targeted therapies to increase bone mineral density
Bisphosphonates to slow the progression of bone loss
Prophylactic or therapeutic anticoagulation therapy (such as warfarin at a stable dose or low-molecular weight heparin)
Inactivated influenza vaccinations
Megestrol acetate administered as an appetite stimulant
Mineralocorticoids (e.g. fludrocortisone)
Inhaled or low-dose corticosteroids administered for chronic obstructive pulmonary disease or asthma
Low-dose mineralocorticoids administered for orthostatic hypotension or low-dose mineralocorticoids and corticosteroids for adrenocortical insufficiency Premedication for pemetrexed and cisplatin or carboplatin is permitted. Premedication with antihistamines, antipyretics, and/or analgesics may be administered for the second and subsequent atezolizumab/pembrolizumab and tiragolumab/placebo infusion only, at the discretion of the investigator.

In general, investigators manage a patient's care with supportive therapies other than those defined as cautionary or prohibited therapies as clinically indicated, per local standard practice. Patients who experience infusion-associated symptoms may be treated symptomatically with acetaminophen, ibuprofen, diphenhydramine, and/or H2-receptor antagonists (e.g., famotidine, cimetidine), or equivalent medications per local standard practice. Serious infusion associated-events manifested by dyspnea, hypotension, wheezing, bronchospasm, tachycardia, reduced oxygen saturation, or respiratory distress are managed with supportive therapies as clinically indicated (e.g., supplemental oxygen and β2-adrenergic agonists)

Cautionary Therapy

Systemic corticosteroids and TNF-α inhibitors may attenuate potential beneficial immunologic effects of treatment with tiragolumab and/or atezolizumab. Therefore, in situations in which systemic corticosteroids or TNF-α inhibitors would be routinely administered, alternatives, including antihistamines, are considered. If the alternatives are not feasible, systemic corticosteroids and TNF-α inhibitors may be administered at the discretion of the investigator.

Systemic corticosteroids are recommended, at the discretion of the investigator, for the treatment of specific adverse events when associated with tiragolumab and/or atezolizumab therapy

Prohibited Therapy

Use of the following concomitant therapies is prohibited as described below:
Investigational therapy (other than protocol-mandated study treatment) is prohibited within 28 days prior to initiation of study treatment and during study treatment.
Concomitant therapy intended for the treatment of cancer (including, but not limited to, chemotherapy, hormonal therapy, immunotherapy, radiotherapy, and herbal therapy) is prohibited for various time periods prior to starting study treatment, depending on the agent, and during study treatment, until disease progression is documented and the patient has discontinued study treatment with the exception of palliative radiotherapy under certain circumstances.
Live, attenuated vaccines within 4 weeks prior to initiation of study treatment, during study treatment, and for 5 months after the final dose of study treatment
Systemic immunomodulatory agents (including, but not limited to, interferons and IL-2) within 4 weeks or 5 drug-elimination half-lives (whichever is longer) prior to initiation of study treatment and during study treatment because these agents could potentially increase the risk for autoimmune conditions when given in combination with study treatment
Systemic immunosuppressive medications (including, but not limited to, cyclophosphamide, azathioprine, methotrexate, and thalidomide) during study treatment because these agents could potentially alter the efficacy and safety of study treatment

D. Inclusion Criteria

Patients must meet the following criteria for study entry:
Signed Informed Consent Form
Age ≥18 years at time of signing Informed Consent Form
Ability to comply with the study protocol
COG Performance Status of 0 or 1
Histologically or cytologically documented locally advanced unresectable or metastatic non-squamous NSCLC

Patients with tumors of mixed NSCLC histology must be classified as non-squamous on the basis of the major histological component. Patients with mixed NSCLC and small cell lung cancer are not eligible for the study.
No prior systemic treatment for metastatic non-squamous NSCLC

Patients who have received prior neo-adjuvant, adjuvant chemotherapy, radiotherapy or chemoradiotherapy with curative intent for non-metastatic disease must have experienced a treatment-free interval of at least 12 months from randomization since the last dose of chemotherapy and/or radiotherapy.
Known tumor PD-L1 status through use of a local health authority-approved assay (SP263 [preferred], or 22C3 only if SP263 is not available) or by central laboratory assay (investigational VENTANA [SP263]CDx Assay) if an approved local test is not available

Confirmed availability of representative tumor specimens in formalin-fixed, paraffin-embedded (FFPE) blocks (preferred) or at least 15-20 unstained serial slides, along with an associated pathology report. If central testing for EGFR mutations and/or ALK translocations are required, an additional 5 unstained slides must be provided.

Tumor tissue is of good quality based on total and viable tumor content (i.e., a minimum number of 100 viable tumor cells with preserved cellular context and tissue architecture). Acceptable samples include samples from resections, core-needle biopsies for deep tumor tissue (with a minimum of three cores for freshly collected biopsies) or excisional, incisional, punch, or forceps biopsies for cutaneous, subcutaneous, or mucosal lesions, or endobronchial ultrasound (EBUS) core needle biopsy.

Endobronchial ultrasound-transbronchial needle aspiration (EBUS-TBNA), which is sometimes referred to as a fine-needle aspiration, is acceptable (particularly if a larger gauge needle is used) provided tissue is of good quality as described above (i.e., a minimum number of 100 viable tumor cells with preserved cellular context and tissue architecture). For needle aspirations, an 18 gauge or larger needle is recommended.

Fine-needle aspirations that do not preserve tissue architecture and yield cell suspension and/or cell smears, brushings, cell pellets from pleural effusions, and lavage samples are not acceptable.

Tumor tissue from bone metastases is not evaluable for tumor PD-L1 expression by IHC and is therefore not acceptable.

If archival tissue is either insufficient or unavailable, the patient may undergo a biopsy at screening if the biopsy site is safely accessible. If a biopsy cannot be provided, the patient may still be eligible upon discussion with the Medical Monitor if ≥10 unstained, serial slides can be provided.
Measurable disease, as defined by RECIST v1.1
   Previously irradiated lesions can only be considered measurable disease if disease progression has been unequivocally documented at that site since radiation and the previously irradiated lesion is not the only site of measurable disease.
Life expectancy ≥12 weeks
Adequate hematologic and end-organ function, defined by the following laboratory test results, obtained within 14 days prior to initiation of study treatment (Day 1 of Cycle 1):
ANC ≥1.5×109/L (≥1500/μL) without granulocyte colony-stimulating factor support
Lymphocyte count ≥0.5×109/L (≥500/μL)
Platelet count ≥100×109/L (≥100,000/μL) without transfusion
Hemoglobin ≥90 g/L (≥9 g/dL)
   Patients may be transfused or receive erythropoietic treatment as per local standard of care to meet this criterion.
AST, ALT, and ALP 2.5× upper limit of normal (ULN), with the following exceptions:
   Patients with documented liver metastases: AST and ALT ≤5×ULN
   Patients with documented liver or bone metastases: ALP ≤5×ULN
Total bilirubin 1.5×ULN with the following exception:
   Patients with known Gilbert disease: bilirubin level 3×ULN
Creatinine clearance (CrCl) 40 mL/min (60 mL/min required for cisplatin), calculated using the Cockcroft-Gault formula (Cockcroft and Gault 1976) or by 24-hour urine collection for determination of CrCl
Albumin ≥25 g/L (≥2.5 g/dL)
For patients not receiving therapeutic anticoagulation: INR and aPTT ≤1.5×ULN
For patients receiving therapeutic anticoagulation: stable anticoagulant regimen
Negative HIV test at screening
Negative hepatitis B surface antigen (HBsAg) test at screening
Positive hepatitis B surface antibody (HBsAb) test at screening, or negative HBsAb at screening accompanied by either of the following:
Negative total hepatitis B core antibody (HBcAb)
Positive total HBcAb test followed by a negative (per local laboratory definition) hepatitis B virus (HBV) DNA test
Negative hepatitis C virus (HCV) antibody test at screening, or positive HCV antibody test followed by a negative HCV RNA test at screening. The HCV RNA test is performed only for patients who have a positive HCV antibody test.
For women of childbearing potential: agreement to remain abstinent (refrain from heterosexual intercourse) or use contraception, and agreement to refrain from donating eggs, as defined below:

Women must remain abstinent or use contraceptive methods with a failure rate of <1% per year during the treatment period and for 90 days after the final dose of tiragolumab or placebo, 5 months after the final dose of atezolizumab or pembrolizumab, 6 months after the final dose of pemetrexed and carboplatin or cisplatin. Women must refrain from donating eggs during this same period.

A woman is considered to be of childbearing potential if she is postmenarcheal, has not reached a postmenopausal state (≥12 continuous months of amenorrhea with no identified cause other than menopause), and is not permanently infertile due to surgery (i.e., removal of ovaries, fallopian tubes, and/or uterus) or another cause as determined by the investigator (e.g., Müllerian agenesis). The definition of childbearing potential may be adapted for alignment with local guidelines or regulations.

Examples of contraceptive methods with a failure rate of <1% per year include bilateral tubal ligation, male sterilization, hormonal contraceptives that inhibit ovulation, hormone-releasing intrauterine devices, and copper intrauterine devices.

The reliability of sexual abstinence is evaluated in relation to the duration of the clinical trial and the preferred and usual lifestyle of the patient.

Periodic abstinence (e.g., calendar, ovulation, symptothermal, or postovulation methods) and withdrawal are not adequate methods of contraception.

Women who would like to become pregnant after discontinuation of study treatment are to seek advice about oocyte preservation prior to initiation of study treatment because of the possibility of irreversible infertility due to treatment with carboplatin.
For men: agreement to remain abstinent (refrain from heterosexual intercourse) or use contraceptive methods, and agreement to refrain from donating sperm, as defined below:

With a female partner of childbearing potential who is not pregnant, men who are not surgically sterile must remain abstinent or use a condom plus an additional contraceptive method that together result in a failure rate of <1% per year during the treatment period and for 90 days after the final dose of tiragolumab or placebo, 4 months after the final dose of atezolizumab or pembrolizumab, 6 months after the final dose of pemetrexed and carboplatin or cisplatin to avoid exposing the embryo and/or genotoxicity. Men must refrain from donating sperm during this same period.

With a pregnant female partner, men must remain abstinent or use a condom during the treatment period and for 90 days after the final dose of tiragolumab or placebo, 4 months after the final dose of atezolizumab or pembrolizumab, 6 months after the final dose of carboplatin and paclitaxel or nab-paclitaxel to avoid exposing the embryo.

The reliability of sexual abstinence is evaluated in relation to the duration of the clinical trial and the preferred and usual lifestyle of the patient.

Periodic abstinence (e.g., calendar, ovulation, symptothermal, or postovulation methods) and withdrawal are not adequate methods of contraception.

Men who would like to father a child after initiation of study treatment are to seek advice about sperm preservation prior to initiation of study treatment because of the possibility of irreversible infertility due to treatment with pemetrexed, cisplatin, or carboplatin.

E. Exclusion Criteria

Patients are excluded from study entry if they have an NSCLC known to have a mutation in the EGFR gene or an ALK fusion oncogene. Patients with non-squamous NSCLC who have an unknown EGFR or ALK status are tested at pre-screening or screening. EGFR and/or ALK status are assessed locally or at a central laboratory. EGFR status assessed locally is performed on tissue or cytology using a validated health authority-approved test that detects mutations in exons 18-21. If samples are submitted for central EGFR and/or ALK testing, additional slides are provided.

F. Assessments

Patients are closely monitored for safety and tolerability throughout the study. Patients are assessed for toxicity prior to each dose; dosing occurs only if the clinical assessment and local laboratory test values are acceptable.

Medical History, Baseline Conditions, Concomitant Medication, and Demographic Data

Medical history, including clinically significant diseases, surgeries, cancer history (including prior cancer therapies and procedures) and lung cancer mutational status (e.g., EGFR and ALK), reproductive status, smoking history, and use of alcohol and drugs of abuse, are recorded at baseline. In addition, all medications (e.g., prescription drugs, over-the-counter drugs, vaccines, herbal or homeopathic remedies, nutritional supplements) used by the patient within 7 days prior to initiation of study treatment are recorded. At the time of each follow-up physical examination, an interval medical history is obtained and any changes in medications and allergies are recorded.

Demographic data include age, sex, and self-reported race/ethnicity.

Physical Examinations

A complete physical examination is performed at screening and include an evaluation of the head, eyes, ears, nose, and throat, and the cardiovascular, dermatologic, musculoskeletal, respiratory, gastrointestinal, genitourinary, and neurologic systems. Any abnormality identified at baseline is recorded.

Limited, symptom-directed physical examinations are performed at specified postbaseline visits and as clinically indicated. Changes from baseline abnormalities are recorded in patient notes. New or worsened clinically significant abnormalities are recorded.

Vital Signs

Vital signs include measurements of respiratory rate, pulse rate, and systolic and diastolic blood pressure while the patient is in a seated position, and temperature.

Record abnormalities observed at baseline on the General Medical History and Baseline Conditions eCRF. At subsequent visits, record new or worsened clinically significant abnormalities.

ECOG Performance Status

Performance status is measured using ECOG Performance Status at baseline, and is assessed at regular intervals throughout the study

Tumor and Response Evaluations

Screening and subsequent tumor assessments must include CT scans of abdomen and chest (with IV contrast unless contraindicated and oral contrast as appropriate per institutional standard). A CT scan with contrast of the pelvis is required at screening and as clinically indicated or as per local standard-of-care at subsequent response evaluations. MRI scans with contrast of the chest, abdomen, and pelvis with a non-contrast CT scan of the chest may be used in patients for whom CT scans with contrast are contraindicated (i.e., patients with iodine-based contrast allergy or impaired renal clearance).

A CT (with contrast) or MRI scan with contrast (if CT contrast is contraindicated) of the head must be done at screening to evaluate CNS metastasis in all patients. If CT with contrast is performed and the presence of brain metastases is considered equivocal, an MRI scan of the brain is required to confirm or refute the diagnosis of CNS metastases at baseline.

If a CT scan for tumor assessment is performed in a positron emission tomography (PET)/CT scanner, the CT acquisition must be consistent with the standards for a full contrast diagnostic CT scan.

Further investigations such as bone scans and CT scans of the neck are to be performed if clinically indicated. At the investigator's discretion, other methods of assessment of measurable disease as per RECIST v1.1 may be used.

Tumor assessments performed as standard-of-care prior to obtaining informed consent and within 28 days of Cycle 1, Day 1 may be used rather than repeating tests. All known sites of disease, including measurable and/or non-measurable disease, must be documented at screening and re-assessed at each subsequent tumor evaluation.

Patients will undergo tumor assessments at baseline and at every 6 weeks (±7 days) for 48 weeks following Day 1 of Cycle 1, regardless of treatment delays. After the completion of the Week 48 tumor assessment, tumor assessment is required every 9 weeks (±7 days) regardless of treatment delays, until radiographic disease progression per RECIST v1.1 (or loss of clinical benefit for patients who continue study treatment after disease progression per RECIST v1.1), withdrawal of consent, death, or study termination, whichever occurs first. At the investigator's discretion, scans may be performed at any time if progressive disease or loss of clinical benefit is suspected.

Response is assessed by the investigator on the imaging modalities detailed above, using RECIST v1.1. The investigator's assessment of overall tumor response at all timepoints is only based on RECIST v1.1. Assessments are performed by the same evaluator if possible to ensure internal consistency across visits. Results must be reviewed by the investigator before dosing at the next cycle.

Study treatment with tiragolumab/placebo and atezolizumab/pembrolizumab may be continued as long as patients are experiencing clinical benefit as assessed by the investigator in the absence of unacceptable toxicity or symptomatic deterioration attributed to disease progression after an integrated assessment of radiographic data, biopsy results (if available), and clinical status. Patients who meet criteria for disease progression per RECIST v1.1 are permitted to continue study treatment if they meet all of the criteria specified and provide written consent.

Patients who discontinue treatment for reasons other than radiographic disease progression per RECIST v1.1 (e.g., toxicity, symptomatic deterioration) will continue scheduled tumor assessments at the frequency described above until radiographic disease progression per RECIST v1.1, withdrawal of consent, death, or study termination, whichever occurs first. Patients who start a new anti-cancer therapy in the absence of radiographic disease progression per RECIST v1.1 will continue tumor assessments at the frequency described above until radiographic disease progression per RECIST v1.1, withdrawal of consent, death, or study termination, whichever occurs first.

Investigator assessment of overall tumor response at all timepoints is only based on RECIST v1.1. The overall tumor assessment is as per iRECIST based on entries for all target lesions, non-target lesions, and new lesions. To facilitate evaluation of response per iRECIST, tumor assessments must be continued after disease progression per RECIST v1.1 for patients who receive study treatment beyond progression. This includes continued measurement of target lesions, evaluation of non-target lesions (including monitoring for further worsening of any non-target lesions that have shown unequivocal progression), and evaluation of any newly identified lesions (including measurements, if lesions are measurable) at all subsequent assessments.

Electrocardiograms

An ECG is required at screening and when clinically indicated. ECGs for each patient are obtained from the same machine wherever possible. Lead placement is as consistent as possible. ECG recordings must be performed after the patient has been resting in a supine position for at least 10 minutes.

For safety monitoring purposes, the investigator must review all ECG tracings. Copies of ECG tracings are kept as part of the patient's permanent study file at the site. Any morphologic waveform changes or other ECG abnormalities must be documented.

Clinical Outcome Assessments

PRO instruments are completed to document the treatment benefit and more fully characterize the clinical profile of tiragolumab and atezolizumab plus carboplatin with paclitaxel/nab-paclitaxel. PRO data are collected using the following instruments: EORTC QLQ-C30, EORTC QLQ-LC13, and a single item from EORTC IL46.

The questionnaires (EORTC QLQ-C30, EORTC QLQ-LC13, and EORTC IL46) are completed at Day 1 of Cycle 1 (baseline) prior to administration of study drug; then at every treatment cycle on Day 1 prior to the administration of study drug through Cycle 4 (i.e., on Cycle 2, Day 1; Cycle 3, Day 1; and Cycle 4, Day 1).

At Cycle 5, the questionnaires are completed at every other study treatment cycle on Day 1 prior to administration of study drug (i.e., on Cycle 5, Day 1; Cycle 7, Day 1; Cycle 9, Day 1; and so forth) until the study treatment discontinuation visit, and at the study treatment discontinuation visit.

Blood Samples for Whole Genome Sequencing or Whole Exome Sequencing

At participating sites, blood samples are collected for DNA extraction to enable WGS or WES to identify variants that are predictive of response to study drug, are associated with progression to a more severe disease state, are associated with acquired resistance to study drug, are associated with susceptibility to developing adverse events, can lead to improved adverse event monitoring or investigation, or can increase the knowledge and understanding of disease biology and drug safety. DNA extracted from blood may be compared with DNA extracted from tissue to identify somatic variants by distinguishing germline variants from somatic variants. The samples may be sent to one or more laboratories for analysis.

Collection and submission of blood samples for WGS or WES is contingent upon the review and approval of the exploratory research by each site's IRB/EC and, if applicable, an appropriate regulatory body.

Genomics is increasingly informing researcher's understanding of disease pathobiology. WGS and WES provide a comprehensive characterization of the genome and exome, respectively, and, along with clinical data collected in this study, may increase the opportunity for developing new therapeutic approaches or new methods for monitoring efficacy and safety or predicting which patients are more likely to respond to a drug or develop adverse events. Data are analyzed in the context of this study but may also be explored in aggregate with data from other studies. The availability of a larger dataset will assist in identification and characterization of important biomarkers and pathways to support future drug development.

Blood samples collected for WGS or WES are to be stored until they are no longer needed or until they are exhausted.

Samples for Research Biosample Repository for Research Biosample Repository

The Research Biosample Repository (RBR) is a centrally administered group of facilities used for the long-term storage of human biological specimens, including body fluids, solid tissues, and derivatives thereof (e.g., DNA, RNA, proteins, peptides). The collection, storage, and analysis of RBR samples facilitates the rational design of new pharmaceutical agents and the development of diagnostic tests, which may allow for individualized drug therapy for patients in the future.

Samples for the RBR are collected from patients who give specific consent to participate in this optional research. RBR samples are analyzed to achieve one or more of the following objectives:
To study the association of biomarkers with efficacy or disease progression
To identify safety biomarkers that are associated with susceptibility to developing adverse events or can lead to improved adverse event monitoring or investigation
To increase knowledge and understanding of disease biology and drug safety
To study drug response, including drug effects and the processes of drug absorption and disposition
To develop biomarker or diagnostic assays and establish the performance characteristics of these assays

Sample Collection

The following samples are stored in the RBR and used for research purposes, including, but not limited to, research on biomarkers related to tiragolumab, atezolizumab, non-squamous NSCLC, or drug safety:
Leftover blood, serum, plasma, and tumor tissue samples (with the exception of remaining archival tissue blocks, which are returned to sites) and any derivatives thereof (e.g., DNA, RNA, proteins, peptides), including leftover tissue samples from medically indicated procedures (e.g., bronchoscopy, esophagogastroduodenoscopy, colonoscopy) performed at the investigator's discretion during the course of the study
Optional stool sample collected pre-dose at Cycle 1, Day 1 and between Cycle 2, Day 15 and pre-dose at Cycle 3, Day 1. These samples may be collected at home.

The above samples may be sent to one or more laboratories for analysis of germline or somatic variants via WGS, WES, or other genomic analysis methods. Genomics is increasingly informing researcher's understanding of disease pathobiology. WGS and WES provide a comprehensive characterization of the genome and exome, respectively, and, along with clinical data collected in this study, may increase the opportunity for developing new therapeutic approaches or new methods for monitoring efficacy and safety or predicting which patients are more likely to respond to a drug or develop adverse events.

Data generated from RBR samples are analyzed in the context of this study but may also be explored in aggregate with data from other studies. The availability of a larger dataset will assist in identification and characterization of important biomarkers and pathways to support future drug development.

G. Treatment and Patient Discontinuation

Treatment Discontinuation

Patients must permanently discontinue study treatment if they experience any of the following:
Intolerable toxicity related to study treatment, including development of an immune-mediated adverse event determined by the investigator to be unacceptable given the individual patient's potential response to therapy and severity of the event
Any medical condition that the investigator determines may jeopardize the patient's safety if he or she continues to receive study treatment
Investigator determination that treatment discontinuation is in the best interest of the patient
Pregnancy
Use of non-protocol-specified anti-cancer therapy
Radiographic disease progression per RECIST v1.1 (unless study treatment is continued beyond radiographic progression)
For patients treated beyond radiographic disease progression, loss of clinical benefit (if treatment with tiragolumab/placebo and atezolizumab/pembrolizumab is continued beyond radiographic disease progression) as determined by the investigator after an integrated assessment of radiographic and biochemical data, local biopsy results (if available), and clinical status (e.g., symptomatic deterioration

The primary reason for study treatment discontinuation is documented on the appropriate eCRF.

Patients who discontinue study treatment prematurely will not be replaced.

Patients return to the clinic for a treatment discontinuation visit 30 days after the final dose of study drug. The visit at which response assessment shows progressive disease may be used as the treatment discontinuation visit. Patients who discontinue study treatment for any reason other than progressive disease or loss of clinical benefit (for patients who continue treatment beyond radiographic disease progression) will continue to undergo tumor response assessments and PRO assessments.

After study treatment discontinuation and disease progression per RECIST v1.1, information on survival follow-up and new anti-cancer therapy is collected via telephone calls, patient medical records, and/or clinic visits approximately every 3 months until death (unless the patient withdraws consent)

H. Analysis

Efficacy Analysis

Efficacy analyses are conducted in the ITT population with patients grouped according to their randomized treatments. Estimates in the difference in ORR between the two treatment arms and PFS HRs are computed along with their 95% CIs.

Hypothesis testing of the co-primary efficacy endpoints will also be conducted in the ITT population.

All co-primary analyses of disease progression and objective response are based on investigator review of tumor assessments using RECIST v1.1.

Primary Efficacy Endpoint

The co-primary efficacy endpoints are confirmed ORR and PFS.

Confirmed ORR is defined as the proportion of patients who have achieved an objective response, characterized by a CR or PR, on two consecutive occasions ≥4 weeks apart.

Objective response is evaluated by treatment arm and patients without post-baseline overall response assessments are counted as non-responders.

An estimate of the difference between the ORR in the two arms is computed along with its 95% CI. The Mantel-Haenszel test is used to compare the ORR between the two treatment arms at the two-sided significance level of 5%, stratified by the protocol-defined stratification factors.

PFS is defined as the time between the date of randomization and the date of first documented disease progression or death, whichever occurs first. Patients who have not experienced disease progression or who have not died at the time of analysis are censored at the time of the last tumor assessment. Patients with no post-baseline tumor assessment are censored at the date of randomization.

PFS is compared between treatment arms with use of the stratified log-rank test.

The HR and its 95% CI for PFS is estimated using a stratified Cox proportional-hazards model.

Kaplan-Meier methodology is used to estimate the median PFS for each treatment arm, and Kaplan-Meier curve is constructed to provide a visual description of the difference between treatment arms.

Secondary Efficacy Endpoints

Overall Survival

OS is defined as the time from randomization to death from any cause. Data for patients who are alive at the time of the data cutoff are censored at the last date they were known to be alive. Data from patients without post-baseline information are censored at the date of randomization. A stratified Cox proportional-hazards model is used to estimate the OS HR and its 95% CI. Kaplan-Meier methodology is used to estimate the OS curve and median OS for each treatment arm.

Duration of Confirmed Response

DOR is defined as the time from the first occurrence of a documented objective response to disease progression or death from any cause, whichever occurs first. The analysis of DOR will include only patients who achieved an objective response to study treatment. DOR is estimated using the Kaplan-Meier methodology. As the determination of DOR is based on a non-randomized subset of patients, no formal hypothesis testing is performed.

Patient Reported Outcomes

TTCD for cough, dyspnea, and chest pain symptoms using the EORTC QLQ-LC13, GHS/QoL, and physical functioning using the EORTC QLQ-C30 is defined as the time from the date of randomization until the first confirmed clinically meaningful deterioration. Confirmed clinically meaningful deterioration in symptoms is defined as a score increase of ≥10-point from baseline in a symptom score that must be held for at least two consecutive assessments or an initial increase ≥10-point from baseline followed by death. Confirmed clinically meaningful deterioration for GHS/QoL and physical functioning is defined as a score decrease of ≥10-point from baseline in GHS/QoL or physical functioning scale score that must be held for at least two consecutive assessments or an initial ≥10-point decrease from baseline followed by death.

For TTCD, data for patients are censored at the last time when they completed an assessment if they have not experienced a confirmed clinically meaningful deterioration event at the clinical cutoff date. If no baseline or post-baseline assessment is performed, patients are censored at the randomization date.

TTCD using the EORTC scale is analyzed using the same methods as for PFS.

Summary statistics and the mean change from baseline of linear-transformed scores are reported for all of the items and subscales of the EORTC QLQ-C30, EORTC QLQ-LC13, and EORTC IL46 (an item for troubled by side effects) questionnaires according to the EORTC scoring manual guidelines.

Safety Analyses

Safety analyses include all treated patients, defined as randomized patients who received any amount of study treatment.

Safety analyses are performed by treatment arm and are based on actual treatment received. Specifically, a patient is included in Arm A in the safety analyses if the patient receives any amount of tiragolumab or atezolizumab, regardless of the initial treatment assignment at randomization.

Drug exposure is summarized, including duration, dosage, and dose intensity. Verbatim description of adverse events is mapped to the MedDRA thesaurus terms.

Severity for all adverse events are graded by the investigator according to the NCI CTCAE v5.0, and severity for CRS will also be graded by the investigator according to the ASTCT consensus grading scale. All adverse events are summarized by treatment arm and NCI CTCAE grade. CRS will also be summarized by treatment arm and ASTCT consensus grade. In addition, serious adverse events and adverse events leading to study treatment discontinuation or interruption are summarized accordingly. Multiple occurrences of the same event are counted once at the maximum severity. Laboratory data with values outside of the normal ranges are identified. Additionally, selected laboratory data, including ADA results, are summarized by treatment arm. Deaths and causes of deaths are summarized.

Pharmacokinetic Analyses

PK samples are collected for PK analysis and to compare exposure in this study with that attained in previous studies. Serum concentrations of tiragolumab and atezolizumab are reported as individual values and summarized (mean, standard deviation, coefficient of variation, median, range, geometric mean, and geometric mean coefficient of variation) by treatment arm and cycle, when appropriate and as data allow.

Individual and median serum tiragolumab and atezolizumab concentrations are plotted by treatment arm and day. Tiragolumab and atezolizumab concentration data may be pooled with data from other studies using an established population PK model to derive PK parameters such as clearance, volume of distribution, and AUC, as warranted by the data. Potential correlations of relevant PK parameters with safety, efficacy, or biomarker outcomes may be explored.

Immunogenicity Analyses

The immunogenicity analyses include patients with any ADA assessments, with patients grouped according to treatment received. The numbers and proportions of treatment-emergent ADA-positive patients and ADA-negative patients for both tiragolumab and atezolizumab are summarized by treatment arm. The relationship between ADA status and safety, efficacy, and PK endpoints may be analyzed and reported via descriptive statistics.

Biomarker Analyses

Biomarker analyses may be performed in an effort to understand the association of relevant markers (e.g., TIGIT) with study treatment efficacy. The efficacy outcomes may be explored in a population of patients whose tumors have high biomarker expression, as determined by IHC and/or RNA analysis. Exploratory analysis of WGS data may be conducted in the context of this study and explored in aggregate with data from other studies to increase researchers' understanding of disease pathobiology and guide the development of new therapeutic approaches. WGS is not applicable for a site that has not been granted regulatory approval for WGS sampling.

Exploratory Analyses

To assess the consistency of the study results in subgroups defined by demographic (e.g., age, sex, and race/ethnicity) and baseline prognostic characteristics (e.g., PD-L1 expression by central laboratory testing, geographic region, and ECOG), the co-primary efficacy endpoints confirmed ORR and PFS in these subgroups are examined. Summaries of confirmed ORR and PFS are produced separately for each level of the categorical variables for comparison between two treatment arms.

Interim Analyses

Periodic analyses of cumulative safety data are planned for this study. Efficacy interim analyses of PFS are conducted when approximately 86 PFS events have been observed in the ITT population. This is projected to occur at approximately 19 months after the first patient is randomized. An early analysis of ORR will also be conducted when approximately 120 patients have had at least two tumor assessments. This is projected to occur at approximately 14 months after the first patient is randomized.

### Example 9. A Phase 11, Open-Label, Multicenter Study Evaluating the Safety and Efficacy of Neoadjuvant and Adjuvant Tiragolumab Plus Atezolizumab, with or without Platinum-Based Chemotherapy, in Patients with Previously Untreated Locally Advanced Resectable Stage II, IIIA, or Select IIIB Non-Small Cell Lung Cancer

A. Overview of Study Design

GO42501 is a global Phase II, open-label, multicenter study evaluating the safety and efficacy of neoadjuvant and adjuvant atezolizumab (Atezo) plus tiragolumab (Tira), with or without platinum-based chemotherapy (Chemo), in patients with previously untreated, histologically or cytologically confirmed resectable Stage II, IIIA, or select IIIB (T3N2 only) NSCLC. The study is designed to establish proof-of-concept clinical data that neoadjuvant treatment with Atezo+Tira or Atezo+Tira+Chemo is safe, tolerable and does not have a clinically significant negative effect on surgical outcomes in patients with early-stage resectable NSCLC. The study is also designed to evaluate potential anti-tumor effects of neoadjuvant Atezo+Tira or Atezo+Tira+Chemo, as measured by major pathological response (MPR). The study is designed with the flexibility to open new cohorts as new treatment combinations become available.

The study treatments administered are provided in Table 55. In this protocol, “study treatment” refers to the combination of treatments assigned to patients as part of this study (i.e., Atezo+Tira, Atezo+Tira+Chemo or chemotherapy).

TABLE 55
Study treatments administered

| Cohort | Neoadjuvant treatment | Adjuvant treatment |
|---|---|---|
| A (PD-L1 high) | Atezo + Tira | Atezo + Tira |
| | | Chemo^{a} |
| B (PD-L1 all comers) | Atezo + Tira + Chemo | Atezo + Tira |

Atezo = atezolizumab;
Chemo = platinum-based chemotherapy;
PD-L1 = programmed death-ligand 1;
Tira = tiragolumab.
^{a}Investigator’s choice to administer chemotherapy as adjuvant treatment.

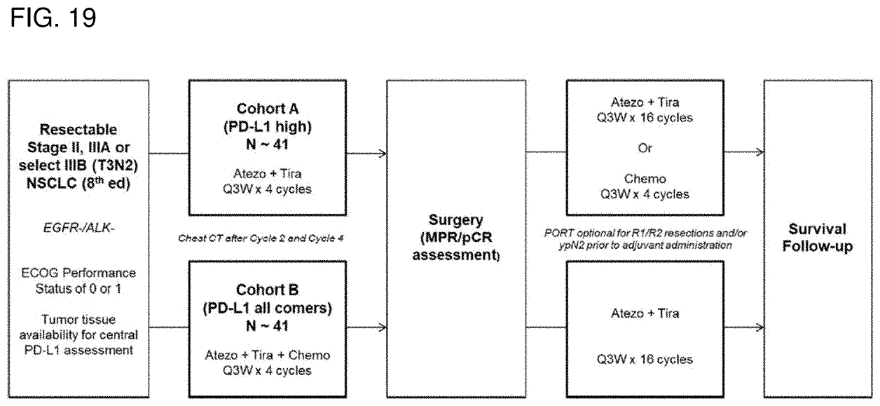

FIG. 19 presents an overview of the study design. Patients are assigned to a cohort on the basis of PD-L1 status and receive treatment as follows:

Cohort A (PD-L1 high): neoadjuvant Atezo+Tira for 4 cycles, followed by surgical resection and either adjuvant Atezo+Tira for 16 cycles or adjuvant chemotherapy for 4 cycles.

Cohort B (PD-L1 all comers): neoadjuvant Atezo+Tira+Chemo for 4 cycles, followed by surgical resection and adjuvant Atezo+Tira for 16 cycles.

Selection of the platinum-based chemotherapy regimen is at the discretion of the investigator, based on histology subtypes and documented at the time of initiation. The following platinum-based chemotherapy options are permitted for this study.
For non-squamous NSCLC:
   Cisplatin+pemetrexed
   Carboplatin+pemetrexed
   Carboplatin+paclitaxel
For squamous NSCLC:
   Cisplatin+gemcitabine
   Carboplatin+gemcitabine
   Carboplatin+paclitaxel

Surgical specimens are assessed for pathological response (MPR and pathological complete response (pCR)) by an independent central pathology laboratory as well as by the investigator's site pathology laboratory. In addition, exploratory biomarker analyses may be performed on leftover surgical specimens (primary tumor tissue and dissected lymph nodes).

Postoperative radiotherapy (PORT) is allowed for patients with confirmed, pathological N2+ disease or positive tumor margins present at the time of surgical resection (ypN2 and/or R1/R2) and must be administered prior to adjuvant Atezo+Tira treatment or after adjuvant platinum-based chemotherapy.

All patients complete scheduled tumor assessments of the chest and abdomen by both computed tomography (CT) and positron emission tomography (PET) at screening and by CT only after Cycles 2 and 4 of neoadjuvant treatment. Tumor assessments continue after surgery until recurrence. If a disease follow-up assessment shows evidence of disease recurrence, it should be confirmed pathologically and/or by unequivocal radiographic evidence from the scan. If a scan shows equivocal findings (e.g., mediastinal nodes measuring <1.5 cm in the short axis, lung parenchymal lesions or visceral lesions measuring <1 cm in the longest diameter), a biopsy should be performed. The biopsy should be performed prior to starting the next anti-cancer therapy. If a biopsy for disease recurrence confirmation is performed, any leftover biopsy tissue is strongly encouraged to be submitted for exploratory biomarker research. If a biopsy is not feasible or safe, confirmatory scans must be performed again within 4 to 8 weeks.

If the biopsy does not show evidence of disease recurrence (e.g., non-malignant infiltrates), the patient may continue with scheduled study treatment, assessments, and/or follow-up. In the absence of disease recurrence, disease follow-up assessments should continue until disease recurrence, withdrawal of consent, death, loss to follow-up, or study termination by the Sponsor, whichever occurs first.

All patients undergo safety, tolerability, and exploratory assessments on Day 1 of each cycle in the neoadjuvant treatment phase (both cohorts) and in the adjuvant treatment phase. After treatment discontinuation, patient follow-up is periodically performed for survival status and any additional anti-cancer treatment. Safety assessments include the incidence, nature, and severity of adverse events, protocol-mandated vital signs, and laboratory abnormalities, and other protocol-specified tests that are deemed critical to the safety evaluation of the study. Adverse events are graded by the investigator according to the National Cancer Institute (NI) Common Terminology Criteria for Adverse Events (CTCAE), Version 5.0 (v 5.0).

Patient samples, including archival and fresh tumor and/or lymph node tissues as well as serum, plasma, and blood, are collected for future exploratory biomarker assessments. In addition, patients are provided an opportunity to consent to optional stool collection for exploratory microbiome research.

Specific objectives and corresponding endpoints for the study are outlined in Table 56.

TABLE 56
Objectives and corresponding endpoints

| | |
|---|---|
| Primary Safety Objectives | Corresponding Endpoints |
| To evaluate the safety of Atezo + Tira as | Incidence and length of surgical delays, |
| neoadjuvant treatment followed by either | incidence of operative and post-operative |
| Atezo + Tira or Chemo as adjuvant treatment. | complications, and/or number of surgical |
| To evaluate the safety of Atezo + Tira + Chemo | cancellations related to study treatment. |
| as neoadjuvant treatment followed by | Incidence and severity of adverse events, with |
| Atezo + Tira as adjuvant treatment. | severity determined according to NCI- |
| | CTCAE v5.0. The severity of CRS |
| | is determined according to the |
| | ASTCT CRS Consensus Grading Scale. |
| Primary Efficacy Objective | Corresponding Endpoint |
| To evaluate the efficacy of Atezo + | MPR rate, defined as the proportion of patients |
| Tira or Atezo + Tira + Chemo | who achieve MPR, with MPR, defined as 10% |
| as neoadjuvant treatment. | residual viable tumor at the time of surgical |
| | resection in the primary tumor, as assessed by |
| | the central pathology laboratory. |
| Secondary Efficacy Objectives | Corresponding Endpoints |
| To evaluate the efficacy of Atezo + | pCR, defined as the absence of any viable |
| Tira or Atezo + Tira + Chemo | primary tumor at the time of surgical resection, as |
| as neoadjuvant treatment. | assessed by the central pathology laboratory. |
| To evaluate the efficacy of Atezo + | EFS, defined as the time from first dose of study |
| Tira as neoadjuvant treatment | drug to any of the following events, whichever |
| followed by either Atezo + Tira or | occurs first: disease progression that precludes |
| Chemo as adjuvant treatment. To evaluate | surgical resection, as assessed by the investigator |
| the efficacy of Atezo + Tira + Chemo as | according to RECIST v1.1, or local or distant |
| neoadjuvant treatment followed by | disease recurrence after surgery, including the |
| Atezo + Tira as adjuvant treatment. | occurrence of a new primary NSCLC, |
| | or death from any cause. |
| Exploratory Safety Objective | Corresponding Endpoints |
| To evaluate the safety of | Extent to which patients are burdened by |
| Atezo + Tira as neoadjuvant | treatment side effects, as assessed through the |
| and adjuvant treatment, with or | use of the single-item EORTC IL46. |
| without Chemo, from the | Presence, frequency of occurrence, severity, |
| patient’s perspective. | and/or interference with selected symptomatic |
| | treatment toxicities, as assessed through the use |
| | of the NCI PRO-CTCAE. |
| Pharmacokinetic Objective | Corresponding Endpoint |
| To characterize the PK profile of atezolizumab | Serum concentrations of atezolizumab and |
| and tiragolumab when given in combination. | tiragolumab at specified timepoints. |
| Immunogenicity Objective | Corresponding Endpoints |
| To evaluate the immune response to | Prevalence of ADAs to tiragolumab |
| atezolizumab and tiragolumab. | at baseline and incidence of ADAs to |
| | tiragolumab during the study. |
| | Prevalence of ADAs to atezolizumab |
| | at baseline and incidence of ADAs to |
| | atezolizumab during the study. |
| Exploratory Immunogenicity Objective | Corresponding Endpoint |
| To evaluate the potential effects of ADAs. | Relationship between atezolizumab and/or |
| | tiragolumab ADA status and efficacy, |
| | safety, or PK endpoints. |
| Exploratory Biomarker Objective | Corresponding Endpoints |
| To identify and/or evaluate biomarkers that are | Relationship between other biomarkers in blood, |
| predictive of response to atezolizumab or | tumor tissue, lymph node and stool and efficacy, |
| tiragolumab (i.e., predictive biomarkers), are early | safety, and pharmacologic measures. |
| surrogates of efficacy, are associated with | Relationship of ctDNA levels and changes |
| progression to a more severe disease state (i.e., | with clinical outcome measures. |
| prognostic biomarkers), are associated with | Relationship between immune cells in the |
| acquired resistance to atezolizumab or | tumor microenvironment and periphery with |
| tiragolumab, are associated with susceptibility to | clinical outcome measures. |
| developing adverse events or may lead to | |
| improved adverse event monitoring or | |
| investigation (i.e., safety biomarkers), can provide | |
| evidence of atezolizumab or tiragolumab activity | |
| (i.e., pharmacodynamic biomarkers), or can | |
| increase the knowledge and understanding of | |
| disease biology and drug safety. | |
| Additional Exploratory Objective | Corresponding Endpoints |
| To evaluate treatment with neoadjuvant and | Change from baseline in patient-reported |
| adjuvant Atezo + Tira, with or without | physical functioning, role functioning, and |
| chemotherapy, from the patients perspective. | GHS/QoL, as measured by the |
| | EORTC-QLQ-C30. |
| | Change from baseline in patient-reported lung |
| | cancer symptoms for cough, dyspnea, and chest |
| | pain, as measured through the use of the |
| | EORTC-QLQ-LC13. |

ADA = anti-drug antibody;
ASTCT = American Society for Transplantation and Cellular Therapy;
Atezo + Tira = atezolizumab plus tiragolumab;
Atezo + Tira + Chemo = atezolizumab plus tiragolumab plus platinum-based chemotherapy;
CRS = cytokine-release syndrome;
ctDNA = circulating tumor DNA;
EFS = event-free survival;
EORTC = European Organisation for Research and Treatment of Cancer;
GHS/QoL = global health status and quality of life;
IL = item list;
MPR = major pathological response;
NCI CTCAE v5.0 = National Cancer Institute Common Terminology Criteria for Adverse Events, Version 5.0;
NSCLC = non-small cell lung cancer;
PBMC = peripheral blood mononuclear cell;
pCR = pathological complete response;
PK = pharmacokinetic;
PRO = patient-reported outcomes;
QLQ-C30 = Quality-of-Life-Questionnaire for Cancer;
QLQ-LC13 = Quality-of-Life-Questionnaire Lung Cancer module;
RECIST v1.1 = Response Evaluation Criteria in Solid Tumors, Version 1.1.

B. Treatment Assignment and Safety Lead-in

Patients must be eligible for R0 resection with curative intent at screening and must meet all eligibility criteria specified herein. Patients who do not meet the criteria for participation in this study (screen failures) may qualify for two re-screening opportunities (for a total of three screenings per participant) at the investigator's discretion.

Treatment Assignment and Safety Lead-in Based on Tumor PD-L1 Status

After providing informed consent, patients undergo screening procedures, including central assessment of PD-L1 status by SP263 immunohistochemistry (IHC) (tumor cell (TC)-based assay.

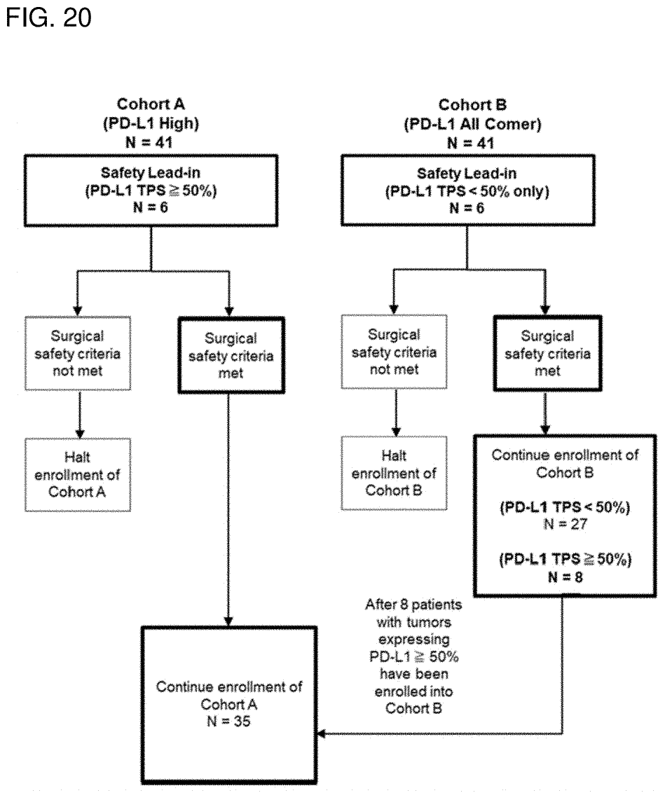

Enrollment is completed in a step-wise manner as follows (see FIG. 20), with approximately 41 patients enrolled in each of the cohorts:

1. Initially there is a Safety Lead-in:
Six patients with tumors having PD-L1 tumor proportion score (TPS) ≥50% at screening are enrolled in Cohort A (PD-L1 high cohort);
Cohort B (PD-L1 all-comer cohort) starts with enrollment of 6 patients with tumors having PD-L1 TPS <50%.
   2. The safety of each cohort is evaluated, and if deemed to be safe:
Patients are enrolled only into Cohort B (PD-L1 all comers), including patients with tumors having PD-L1 TPS ≥50%, until 8 patients with tumors having PD-L1 TPS ≥50% have been enrolled in that cohort.
The remainder of Cohort B patients then comprise patients with tumors having PD-L1 TPS <50%.
   3. After 8 patients total with tumors having PD-L1 TPS E 50% have been enrolled in Cohort B (PD-L1 all comers), enrollment to Cohort A (PD-L1 high) is resumed, and all subsequent patients with tumors having PD-L1 TPS ≥50% are enrolled in that cohort.

Safety Lead-in

To account for potential surgical delays, cancellations or complications related to study treatment, enrollment within each cohort is suspended to allow for a safety evaluation after approximately 6 patients have completed neoadjuvant treatment and either completed surgery or had their surgery plan changed. The cohorts may not enroll at the same speed, and each cohort is evaluated separately and independently.

The safety evaluation is based on safety and surgical data. Based on review of the data, it is recommended either that enrollment into that cohort s continued or the cohort is closed. During the safety lead-in period, a patient may be replaced if he or she does not proceed to surgery for reasons other than an adverse event of special interest or disease progression.

Surgery and Adjuvant Treatment

Patients undergo surgical resection of their tumor upon completion of four cycles of neoadjuvant therapy. Prior to the surgery, the attending surgeon and medical oncologist reassess the patient.

The pre-surgery visit should occur within 30 days after the last dose of neoadjuvant treatment; repeat pulmonary function tests (PFTs) (if clinically indicated), as well as associated assessments should be performed in accordance with local institutional practice. The surgical procedure should be performed within 30 days after the pre-surgery visit if judged clinically feasible by both the attending surgeon and medical oncologist.

Patients who are found to have disease progression at scheduled tumor assessments (after Cycle 2 and Cycle 4) or at any time during neoadjuvant treatment and are still deemed resectable and non-metastatic proceed to surgery if amenable and remain eligible for all study treatment and evaluations.

The Medical Monitor must be consulted if an investigator plans for a patient to proceed to surgery in the absence of disease progression before completing all four cycles of neoadjuvant treatment.

Patients who discontinue neoadjuvant treatment early because of disease progression and do not proceed to surgery are discontinued from additional in-clinic study procedures and proceed to receive other treatment as determined by the investigator. Such patients remain in the study for survival follow-up. For patients who are responding to neoadjuvant therapy but cannot proceed to surgery due to an unforeseen medical issue (e.g., pulmonary embolism or myocardial infarction), the patient can continue protocol-specified treatment, such as chemotherapy (Cohort A [PD-L1 high]) or Atezo+Tira (Cohort A or B) and radiotherapy.

After surgical resection, patients continue to receive adjuvant Atezo+Tira or adjuvant chemotherapy (Cohort A [PD-L1 high] only) until one of the following occurs: administration of up to four cycles of adjuvant chemotherapy per local standard of care (SOC), 16 cycles of adjuvant Atezo+Tira, unacceptable toxicity, disease recurrence, death, or patient and/or physician decision to discontinue study treatment.

End of Study and Length of Study

The end of this study is defined as the date when the last patient, last visit occurs, which will occur approximately 3 years after the last patient receives the final dose of study drugs after surgery. The total length of the study, from screening of the first patient to the end of the study, will be approximately 5-6 years.

C. Rationale for Study Design

This study evaluates the surgical safety and feasibility of Atezo+Tira or Atezo+Tira+Chemo as neoadjuvant treatment for patients with previously untreated locally advanced NSCLC. The study also evaluates the efficacy, pharmacokinetics, immunogenicity, and safety of neoadjuvant Atezo+Tira or Atezo+Tira+Chemo followed by adjuvant Atezo+Tira or adjuvant chemotherapy.

Rationale for Atezolizumab+Tiragolumab Dose and Schedule

Atezolizumab is administered at a fixed dose of 1200 mg Q3W (1200 mg on Day 1 of each 21-day cycle), which is an approved dosage for atezolizumab, as outlined in the prescribing information. Anti-tumor activity has been observed across doses ranging from 1 mg/kg to 20 mg/kg Q3W. In Study GO27381 (PCD4989g), the maximum tolerated dose of atezolizumab was not reached, and no dose limiting toxicities (DLTs) were observed at any dose. The fixed dose of 1200 mg Q3W (equivalent to an average body weight-based dose of 15 mg/kg Q3W) was selected on the basis of both nonclinical studies (Deng et al., MAbs, 8: 593-603, 2016) and available clinical pharmacokinetic (PK), efficacy, and safety data.

Tiragolumab is administered at a fixed dose of 600 mg intravenously Q3W on Day 1 of each 21-day cycle. The fixed dose of tiragolumab 600 mg IV Q3W was selected on the basis of available clinical pharmacokinetics, efficacy, and safety data from Study GO30103, in which patients received single agent tiragolumab or tiragolumab plus atezolizumab. In Study GO30103, the maximum tolerated dose was not reached, and no DLTs were observed with tiragolumab monotherapy or in combination with atezolizumab 1200 mg Q3W (tiragolumab dose range of 2-1200 mg Q3W). Complete occupancy of peripheral TIGIT receptors on CD4+, CD8+, and NK cells was observed beginning at tiragolumab dose level of 30 mg Q3W and remained sustained at all higher doses. Anti-tumor activity (as assessed by radiographic partial responses (PRs)) was observed for tiragolumab at a dose range of 30-600 mg Q3W when given in combination with atezolizumab 1200 mg Q3W.

In the Phase II study GO40290, all patients who were enrolled in the atezolizumab plus tiragolumab arm received 600 mg tiragolumab. At this dose, tiragolumab was tolerated and the combination of atezolizumab plus tiragolumab resulted in a clinically meaningful improvement in PFS and a higher ORR compared with placebo in combination with atezolizumab. Given the favorable benefit-risk ratio observed at 600 mg, this same dose of tiragolumab is used for this study.

Rationale for Patient Population

The study enrolls patients with resectable Stage II, IIIA, and select IIIB (T3N2 only) NSCLC as determined at screening. Patients with tumors having high PD-L1 expression (TPS ≥50%, as determined by immunohistochemistry [IHC] by SP263 at a central laboratory) are enrolled in Cohort A (PD-L1 high), while all comers (regardless of PD-L1 expression level) are enrolled in Cohort B (PD-L1 all comers).

Neoadjuvant and adjuvant chemotherapy has shown significant but modest benefit for patients with early-stage resectable NSCLC, but there is still a substantial unmet need for improvement in outcomes in this treatment setting.

Tumor-cell killing by cytotoxic chemotherapy may expose the immune system to high levels of tumor antigens. Boosting tumor-specific T-cell immunity in this setting by blocking the PD-L1 and TIGIT pathways may result in deeper and more durable responses than those observed with standard chemotherapy alone (Merritt et al., J Thorac Cardiovasc Surg, 126: 1609-1617, 2003; Apetoh et al., Nat Med, 13: 1050-1059, 2007), and this may reasonably occur in tumors regardless of PD-L1 expression.

Given the strong data from CITISCAPE showing the magnitude of benefit in the PD-L1 TPS ≥50% population, and the need to improve survival and decrease recurrence rates for patients with resectable early-stage NSCLC, this study examines the efficacy of Atezo+Tira combination as a chemotherapy-free option. A chemotherapy-free option for patients with resectable NSCLC would be a landmark improvement for patients and may spare patients the early and late toxicity associated with platinum-based chemotherapy.

Rationale for major pathological response as primary endpoint

The primary efficacy objective of the study is to evaluate the efficacy of neoadjuvant treatment with Atezo+Tira or Atezo+Tira+Chemo in patients with resectable Stage II, IIIA, or select IIIB (T3N2 only) NSCLC as measured by central pathology laboratory-assessed major pathological response (MPR).

While overall survival (OS) is the standard in evaluating clinical benefit in adjuvant and neoadjuvant NSCLC trials, readout of OS often takes many years, especially in early-stage disease. Thus, adopting meaningful surrogate endpoints may expedite the evaluation of new therapies and bring new treatments to NSCLC patients sooner. Pathological response after surgical resection of NSCLC has been proposed as a surrogate endpoint for OS (Hellmann et al., Lancet Oncol, 15: e42-50, 2014). Hellmann et al. (2014) cited the U.S. Food and Drug Administration's (FDA's) definition of the surrogate endpoint in which the endpoint should be “reasonably likely to predict clinical benefit”. They advocate that using pathological response in the neoadjuvant setting meets this definition based on three findings: 1) pathological response strongly correlates to OS; 2) pathological response is reflective of neoadjuvant chemotherapy; and 3) the degree of pathological response correlates with the degree of OS benefit.

Use of pathological response as a surrogate endpoint is not without precedent. Trials for breast cancer have used pathological complete response (pCR) to evaluate the efficacy of neoadjuvant treatment. However, pCR rates have varied in neoadjuvant NSCLC studies. Furthermore, relatively low pCR rates reported in NSCLC may not translate into a clinically significant OS benefit, hence restricting the utility of pCR as a surrogate survival endpoint in NSCLC trials. In fact, few trials have reported corollary survival data for pCR in NSCLC because of low pCR rates (median rate 4%) (Hellmann et al., Lancet Oncol, 15: e42-50, 2014).

Instead of pCR, Hellman et al. proposed the use of MPR, defined as 10% residual viable tumor tissue, as a survival surrogate for patients with resectable NSCLC receiving neoadjuvant chemotherapy treatment (Hellmann et al., Lancet Oncol, 15: e42-50, 2014). This is based on studies in which investigators, in acknowledgment of the rarity of pCR in NSCLC, considered other definitions of pathological response, including residual viable tumor as a surrogate survival endpoint. Junker et al. (J Cancer Res Clin Oncol, 123: 469-477,1997) performed pathological analysis of 40 tumors from patients with Stage IIIA and IIIB disease who were given sequential neoadjuvant chemotherapy treatment, chemoradiotherapy, and surgical resection. Patients with ≤10% residual tumor in this group had a median survival of 36 months, while patients who had >10% residual viable tumor tissue had a median survival of 14 months.

Two prospective trials reported an MPR rate of approximately 22% with neoadjuvant chemotherapy in NSCLC. The first study noted that of the 90 patients with Stage IIIA disease who received neoadjuvant chemotherapy (cisplatin+docetaxel), the median pathological response (amount of tumor necrosis and fibrosis) was 60%, with a median OS of 61 months compared with 22 months in patients with 60% pathological response (Betticher et al., Br J Cancer, 94(8): 1099-1106, 2006). Another study showed that of the 50 patients who received neoadjuvant chemotherapy (cisplatin +docetaxel) in combination with bevacizumab, patients with MPR had a significantly longer 3-year survival rate compared with patients who did not achieve MPR (61 months vs. 22 months, respectively) (Chaft et al., J Thorac Oncol, 8: 1084-1090, 2013). A retrospective study from the MD Anderson Cancer Center by William and colleagues (William et al., J Thorac Oncol, 8: 222-228, 2013) showed that in 160 patients who received neoadjuvant platinum-based chemotherapy, MPR was a stronger predictor of OS than clinical Response Evaluation Criteria in Solid Tumors (RECIST) response.

The Sponsor believes that there is a significant rationale for the use of MPR as a surrogate endpoint based on its correlation to the magnitude of improvement in OS. Currently, there are ongoing global Phase III neoadjuvant registrational trials of chemotherapy plus PD-L1/PD-1 inhibitors vs. chemotherapy alone assessing MPR as a primary or secondary objective. Use of MPR as a surrogate endpoint has the potential to increase the effectiveness of clinical studies and to accelerate new therapies for patients with NSCLC.

### Example 10. Materials and Methods for GO42501 Study

Approximately 82 patients with Stage II, IIIA, or select IIIB (T3N2 only) are enrolled in the GO42501 study.

A. Inclusion Criteria

Patients must meet the inclusion criteria listed below to be eligible for study entry.
Age ≥18 years at time of signing Informed Consent Form.
Ability to comply with the study protocol.
Histologically or cytologically confirmed Stage II, IIIA, or select IIIB (T3N2 only) non-small cell lung cancer (NSCLC) of squamous or non-squamous histology. Staging should be based on the 8th edition of the American Joint Committee on Cancer/Union Internationale Contre le Cancer NSCLC staging system.
   Patients with T4 primary NSCLC are eligible only on the basis of size (tumors >7 cm). Patients with invasion of the diaphragm, mediastinum, heart, great vessels, trachea, recurrent laryngeal nerve, esophagus, vertebral body, carina, and separate tumor nodules in a different ipsilateral lobe are not eligible.
   Patients with NSCLC of mixed histology (squamous and non-squamous) are eligible, and must be classified on the basis of the major histological component (non-squamous or squamous).

Patients may be screened based on clinical stage, but mandatory preoperative documentation of N2 nodal involvement by invasive mediastinal staging (e.g., CT-guided biopsy, endobronchial ultrasound, mediastinoscopy) is required for PET-positive N2 nodes. Pre-operative staging of level 5 and level 6 nodes is optional.
Solid or subsolid appearance of NSCLC on CT scan with no appearance of purely ground-glass opacity (GGO). For subsolid lesions, the tumor size (i.e., clinical T stage) should be measured based on solid component only, exclusive of the GGO component.
Eligible for R0 resection with curative intent at the time of screening, as confirmed by the operating attending surgeon and involved medical oncologist prior to study enrollment.
Adequate pulmonary function to be eligible for surgical resection with curative intent, as assessed by pulmonary function tests (PFTs) performed within 6 months of planned resection and repeated at screening, if clinically indicated, including lung volumes, spirometry, and a diffusion capacity; and meeting at least one of the following criteria:
   Predicted postoperative (ppo) forced expiratory volume in 1 second (FEV1) and ppo diffusion capacity of the lung for carbon monoxide (DL_{CO})≥40%.
   Maximal oxygen consumption (VO₂max) ≥15 mL/kg/min.
   If either ppoFEV1 or ppoDL_{CO} is <40% or a pneumonectomy is planned, cardiopulmonary exercise testing must be performed and VO₂max≥15 mL/kg/min.
   f PFTs were performed before 6 months of planned resection or have not been previously performed, they must be performed during the screening period.
Eligible to receive a platinum-based chemotherapy regimen. The Warnings and Precautions section in the prescribing information of each individual chemotherapy treatment (i.e., carboplatin, cisplatin, pemetrexed, paclitaxel, or gemcitabine) in the intended regimen for each patient are used to determine eligibility.
Measurable disease, as assessed by the investigator per RECIST v1.1.
Availability of a representative tumor specimen that is suitable for determination of PD-L1 status and other exploratory biomarkers via central testing.
Eastern Cooperative Oncology Group (ECOG) Performance Status of 0 or 1.
Normal life expectancy, excluding lung cancer mortality risk.
Adequate hematologic and end-organ function, defined by the following laboratory test results, obtained within 14 days prior to initiation of study treatment:
   Absolute neutrophil count (ANC) ≥1.5×10⁹/L (1500/μL) without granulocyte colony-stimulating factor support.
   Lymphocyte count ≥0.5×10⁹/L (500/μL).
   Platelet count ≥100×10⁹/L (100,000/μL) without transfusion.
   Hemoglobin ≥90 g/L (9 g/dL). Patients may be transfused to meet this criterion.
   Aspartate transaminase (AST), alanine transaminase (ALT), and alkaline phosphatase (ALP) ≤2.5×upper limit of normal (ULN).
   Total bilirubin ≤1.5×ULN with the following exception: Patients with known Gilbert disease: total bilirubin 3×ULN.
   Creatinine clearance ≥45 mL/min (calculated using the Cockcroft-Gault formula). For patients intended to receive cisplatin: creatinine clearance 60 mL/min.
   Serum albumin ≥25 g/L (2.5 g/dL)
   For patients not receiving therapeutic anticoagulation: INR or aPTT ≤1.5×ULN
For patients receiving therapeutic anticoagulation: stable anticoagulant regimen.
Negative HIV test at screening.
Negative hepatitis B surface antigen (HBsAg) test at screening.
Positive hepatitis B surface antibody (HBsAb) test at screening, or negative HBsAb at screening accompanied by either of the following:
   Negative total hepatitis B core antibody (HBcAb).
   Positive total HBcAb test followed by quantitative hepatitis B virus (HBV) DNA <500 IU/mL.
The HBV DNA test is performed only for patients who have a negative HBsAg test, a negative HBsAb test, and a positive total HBcAb test.
Negative hepatitis C virus (HCV) antibody test at screening, or positive HCV antibody test followed by a negative HCV RNA test at screening. The HCV RNA test is performed only for patients who have a positive HCV antibody test.
For women of childbearing potential: agreement to remain abstinent (refrain from heterosexual intercourse) or use contraception, and agreement to refrain from donating eggs.
For men: agreement to remain abstinent (refrain from heterosexual intercourse) or use a condom, and agreement to refrain from donating sperm.

B. Exclusion Criteria

Patients who meet any of the criteria listed below are excluded from study entry.
NSCLC with histology of large cell neuroendocrine carcinoma, sarcomatoid carcinoma, or NSCLC not otherwise specified.
Small cell lung cancer (SCLC) histology or NSCLC with any component of SCLC.
Illness or condition that may interfere with a patient's capacity to understand, follow, and/or comply with study procedures.
Any prior therapy for lung cancer, including immunotherapy, chemotherapy, or radiotherapy.
Active or history of autoimmune disease or immune deficiency, including, but not limited to, myasthenia gravis, myositis, autoimmune hepatitis, systemic lupus erythematosus, rheumatoid arthritis, inflammatory bowel disease, antiphospholipid antibody syndrome, Wegener granulomatosis, Sjögren syndrome, Guillain-Barré syndrome, or multiple sclerosis, with the following exceptions: patients with a history of autoimmune-related hypothyroidism who are on thyroid-replacement hormone are eligible for the study. Patients with controlled Type 1 diabetes mellitus who are on an insulin regimen are eligible for the study. Patients with eczema, psoriasis, lichen simplex chronicus, or vitiligo with dermatologic manifestations only (e.g., patients with psoriatic arthritis are excluded) are eligible for the study provided all of following conditions are met:
   Rash must cover <10% of body surface area.
   Disease is well controlled at baseline and requires only low-potency topical corticosteroids.
   No occurrence of acute exacerbations of the underlying condition requiring psoralen plus ultraviolet A radiation, methotrexate, retinoids, biologic agents, oral calcineurin inhibitors, or high-potency or oral corticosteroids within the previous 12 months.
History of idiopathic pulmonary fibrosis, organizing pneumonia (e.g., bronchiolitis obliterans), drug-induced pneumonitis, or idiopathic pneumonitis, or evidence of active pneumonitis on screening chest CT scan.
Active tuberculosis.
Significant cardiovascular disease (such as New York Heart Association Class II or greater cardiac disease, myocardial infarction, or cerebrovascular accident) within 3 months prior to initiation of study treatment, unstable arrhythmia, or unstable angina.
Major surgical procedure, within 4 weeks prior to initiation of study treatment, or anticipation of need for a major surgical procedure during the study.
NSCLC with an activating EGFR mutation or ALKfusion oncogene. For patients with non-squamous NSCLC histology: unknown EGFR and/or ALKstatus requires testing at screening. ALKand/or EGFR status may be assessed locally or submitted for central laboratory testing.
Known c-ros oncogene 1 (ROS1) rearrangement. ROS1 testing at screening is not required for study inclusion; however, patients with known ROS1 rearrangements are excluded.
History of malignancy other than NSCLC within 5 years prior to screening, with the exception of malignancies with a negligible risk of metastasis or death (e.g., 5-year OS rate >90%), such as adequately treated carcinoma in situ of the cervix, non-melanoma skin carcinoma, localized prostate cancer, ductal carcinoma in situ, or Stage I uterine cancer.
Severe infection within 4 weeks prior to initiation of study treatment, including, but not limited to, hospitalization for complications of infection, bacteremia, or severe pneumonia, or any active infection that, in the opinion of the investigator, could impact patient safety.
Treatment with therapeutic oral or IV antibiotics within 2 weeks prior to initiation of study treatment. Patients receiving prophylactic antibiotics (e.g., to prevent a urinary tract infection or chronic obstructive pulmonary disease exacerbation) are eligible for the study.
Prior allogeneic stem cell or solid organ transplantation.
Any other disease, metabolic dysfunction, physical examination finding, or clinical laboratory finding that contraindicates the use of an investigational drug, may affect the interpretation of the results, or may render the patient at high risk from treatment complications
Treatment with a live, attenuated vaccine within 4 weeks prior to initiation of study treatment, or anticipation of need for such a vaccine during study treatment, within 90 days after the final dose of tiragolumab, or within 5 months after the final dose of atezolizumab. Patients being treated with chemotherapy (i.e., carboplatin, cisplatin, pemetrexed, paclitaxel, or gemcitabine) should not receive live vaccines.
Current treatment with anti-viral therapy for HBV.
Positive Epstein-Barr virus (EBV) viral capsid antigen immunoglobulin M (IgM) test at screening. An EBV PCR test should be performed as clinically indicated to screen for acute infection or suspected chronic active infection. Patients with a positive EBV PCR test are excluded.
Treatment with investigational therapy within 42 days prior to initiation of study treatment.
Prior treatment with CD137 agonists or immune checkpoint blockade therapies, including anti-CTLA-4, anti-PD-1, anti-TIGIT, and anti-PD-L1 therapeutic antibodies.
Treatment with systemic immunostimulatory agents (including, but not limited to, interferon and IL-2) within 4 weeks or 5 drug-elimination half-lives (whichever is longer) prior to initiation of study treatment.
Treatment with systemic immunosuppressive medication (including, but not limited to, corticosteroids, cyclophosphamide, azathioprine, methotrexate, thalidomide, and anti-tumor necrosis factor-α [TNF-α] agents) within 2 weeks prior to initiation of study treatment, or anticipation of need for systemic immunosuppressive medication during study treatment, with the following exceptions:
   Patients who received acute, low-dose systemic immunosuppressant medication or a one-time pulse dose of systemic immunosuppressant medication (e.g., 48 hours of corticosteroids for a contrast allergy) are eligible for the study.
   Patients who received mineralocorticoids (e.g., fludrocortisone), corticosteroids for chronic obstructive pulmonary disease (COPD) or asthma, or low-dose corticosteroids for orthostatic hypotension or adrenal insufficiency are eligible for the study.
History of severe allergic anaphylactic reactions to chimeric or humanized antibodies or fusion proteins.
Known hypersensitivity to Chinese hamster ovary cell products or to any component of the atezolizumab or tiragolumab formulation.
Known allergy or hypersensitivity to any component of the chemotherapy regimen the patient may receive during the study.
Pregnancy or breastfeeding, or intention of becoming pregnant during study treatment, within 90 days after the final dose of tiragolumab, 5 months after the final dose of atezolizumab, or 6 months after the final dose of pemetrexed, gemcitabine, paclitaxel, carboplatin, or cisplatin. Women of childbearing potential must have a negative serum pregnancy test result within 14 days prior to initiation of study treatment.

C. Treatment Assignment

The study is an open-label study.

Patients who have high PD-L1 expression (TPS ≥50%, as determined by SP263 IHC at central laboratory) are enrolled in Cohort A (PD-L1 high) while all comers (regardless of PD-L1 expression level) are enrolled in the Cohort B (PD-L1 all comers). Enrollment is completed in a step-wise manner as follows (see FIG. 20), with approximately 41 patients enrolled in each of the cohorts:

1. Initially there is a safety lead-in:
Six patients with tumors having PD-L1 TPS ≥50% at screening are enrolled in Cohort A (PD-L1 high cohort);
Cohort B (PD-L1 all-comer cohort) starts with enrollment of 6 patients with tumors having PD-L1 TPS <50%.
2. The safety of each cohort is evaluated, and if deemed to be safe:
Patients are enrolled only into Cohort B (PD-L1 all comers), including patients with tumors having PD-L1 TPS ≥50%, until 8 patients with tumors having PD-L1 TPS ≥50% have been enrolled in that cohort.
The remainder of Cohort B patients then comprise patients with tumors having PD-L1 TPS <50%.

3. After 8 patients total with tumors having PD-L1 TPS ≥50% have been enrolled in Cohort B (PD-L1 all comers), enrollment to Cohort A (PD-L1 high) is resumed, and all subsequent patients with tumors having PD-L1 TPS ≥50% are enrolled in that cohort.

D. Study Treatment Dosage, Administration, and Compliance

Each study treatment component is administered at the doses and frequency specified in Table 57.

TABLE 57
Study treatment component regimens

| Study Treatment Component | Timing |
|---|---|
| Neoadjuvant Treatment Phase | |
| Cohort A (PD-L1 high) | |
| Atezolizumab (1200 mg IV) | Day 1 of each 21-day cycle for 4 cycles |
| Tiragolumab (600 mg IV) | Day 1 of each 21-day cycle for 4 cycles |
| Cohort B (PD-L1 all comers) | |
| Atezolizumab (1200 mg IV) | Day 1 of each 21-day cycle for 4 cycles |
| Tiragolumab (600 mg IV) | Day 1 of each 21-day cycle for 4 cycles |
| Carboplatin (IV, initial target AUC | Day 1 of each 21-day cycle for 4 cycles |
| of 5 or 6 mg/mL/min)^{a} | |
| Cisplatin (75 mg/m² IV) | Day 1 of each 21-day cycle for 4 cycles |
| Pemetrexed (500 mg/m² IV) | Day 1 of each 21-day cycle for 4 cycles |
| Gemcitabine (1000 or 1250 mg/m² IV)^{b} | Days 1 and 8 of each 21-day cycle for 4 cycles |
| Paclitaxel (175 or 200 mg/m₂ IV)^{c} | Day 1 of each 21-day cycle for 4 cycles |
| Adjuvant Treatment Phase | |
| Cohort A (PD-L1 high) | |
| Atezolizumab (1200 mg IV) | Day 1 of each 21-day cycle for 16 cycles |
| Tiragolumab (600 mg IV) or | Day 1 of each 21-day cycle for 16 cycles |
| Platinum-based chemotherapy | Day 1 of each 21-day cycle for up to 4 cycles |
| Cohort B (PD-L1 all comers) | |
| Atezolizumab (1200 mg IV) | Day 1 of each 21-day cycle for 16 cycles |
| Tiragolumab (600 mg IV) | Day 1 of each 21-day cycle for 16 cycles |

AUC = area under the concentration-time curve;
IV = intravenous.
^{a}Carboplatin should be administered at initial target of AUC 5 mg/mL/min when given after pemetrexed or gemcitabine, and administered at initial target of AUC 6 mg/mL/min when given after paclitaxel.
^{b}Gemcitabine should be administered at 1000 mg/m² when given before carboplatin, and administered at 1250 mg/m² when given before cisplatin.
^{c}Paclitaxel should be administered at 175 mg/m² for patients of Asian race/ethnicity and at 200 mg/m² for patients of non-Asian race/ethnicity.

The neoadjuvant treatment phase consists of four cycles of Atezo+Tira or Atezo+Tira+Chemo. Each cycle is 21 days in duration. On Day 1 of each cycle, all eligible patients are administered study drug infusions in the following order:
Cohort A (PD-L1 high): (1) atezolizumab, (2) tiragolumab.
Cohort B (PD-L1 all comers): Investigator administers one of the listed regimens based on histology subtype and local SOC.
   (1) Atezolizumab, (2) tiragolumab, (3) paclitaxel, (4) carboplatin
   (1) Atezolizumab, (2) tiragolumab, (3) pemetrexed, (4) (carboplatin or cisplatin)
   (1) Atezolizumab, (2) tiragolumab, (3) gemcitabine, (4) (carboplatin or cisplatin)

Atezolizumab (1200 mg) and tiragolumab (600 mg) are administered at a fixed dose. Patients receive their first dose of study treatment on the day of enrollment if possible. If treatment on the day of enrollment is not possible, the first dose occurs no later than 5 days after enrollment.

For Cycle 1, premedication for primary prophylaxis (antihistamines, antipyretics, and/or analgesics) of atezolizumab and tiragolumab is not permitted. Patients should receive anti-emetics and IV hydration for the selected chemotherapy backbone regimen according to the local SOC and manufacturer's instruction. However, because of the immunomodulatory effects of corticosteroids, premedication with corticosteroids should be minimized to the extent that is clinically feasible.

Adjuvant treatment with Atezo+Tira begins within 14 to 60 days after surgery or within 7-42 days after the last PORT treatment. If adjuvant chemotherapy is to be administered, treatment begins within 28-72 days after surgery followed by PORT (if chosen) to begin within 21-60 days after adjuvant chemotherapy. During post-operative adjuvant treatment, infusions are administered in the following order (one regimen per patient). The choice of Cohort A (PD-L1 high) regimen is at the investigator's discretion for each patient.
Cohort A (PD-L1 high): The investigator administers one of the regimens listed below.
   (1) Atezolizumab, (2) tiragolumab
   (1) Paclitaxel, (2) carboplatin
   (1) Pemetrexed, (2) (carboplatin or cisplatin)
   (1) Gemcitabine, (2) (carboplatin or cisplatin)
Cohort B (PD-L1 all comers): (1) atezolizumab, (2) tiragolumab

Atezolbzumab and Tiragolumab

Atezolizumab is administered by IV infusion at a fixed dose of 1200 mg on Day 1 of each 21-day cycle. The dose of atezolizumab is fixed and is not dependent on body weight. Dose reductions are not allowed.

Following the administration of atezolizumab and an observation period, patients receive 600 mg tiragolumab administered by IV infusion on Day 1 of each 21-day cycle. The tiragolumab dose is fixed and is not dependent on body weight. Dose reductions are not allowed.

Administration of study treatment is performed in a monitored setting where there is immediate access to trained personnel and adequate equipment and medicine to manage potentially serious reactions. Atezolizumab and tiragolumab infusions are administered per the instructions outlined in Table 58.

TABLE 58
Study treatment component regimens

| Study drug | First infusion | Subsequent infusions |
|---|---|---|
| Atezolizumab | No premedication | If the patient experienced an |
| infusion | is permitted prior to the | IRR with any previous infusion |
| | atezolizumab infusion. | of atezolizumab, premedication |
| | Vital signs (pulse rate, | with an antihistamine and/or |
| | respiratory rate, blood pressure, | antipyretic medication may be |
| | and temperature) are | administered for subsequent |
| | recorded within 60 minutes | doses at the discretion |
| | prior to the infusion. | of the investigator. |
| | Atezolizumab is infused | Vital signs are recorded within |
| | over 60 (±15) minutes. | 60 minutes prior to the infusion. |
| | If clinically indicated, vital signs | Atezolizumab is infused over |
| | are recorded every 15 (±5) | 30 (±10) minutes if the previous |
| | minutes during the infusion. | infusion was tolerated without |
| | | an IRR or 60 (±15) minutes if |
| | | the patient experienced an IRR |
| | | with the previous infusion. |
| | | If the patient experienced an |
| | | IRR with the previous infusion, |
| | | or if clinically indicated, |
| | | vital signs are recorded |
| | | during the infusion. |
| Observation | After the infusion of | If the patient tolerated the |
| period after infusion | atezolizumab, the patient begins | previous atezolizumab infusion |
| of atezolizumab | a 60-minute observation period, | well without infusion-associated |
| | Vital signs are recorded at 30 | adverse events, the observation |
| | (±10) minutes after the infusion | Period after the next and |
| | of atezolizumab. | following infusions may be |
| | Patients are informed about | reduced to 30 minutes. |
| | the possibility of delayed post- | If the patient experienced |
| | infusion symptoms and | infusion-associated adverse |
| | instructed to contact their study | events in the previous infusion, |
| | physician if they | the observation period |
| | develop such symptoms. | should be 60 minutes. |
| | | If clinically indicated, vital signs |
| | | are recorded at 30 (±10) |
| | | minutes after the infusion |
| | | of atezolizumab. |
| Infusion of | No premedication is permitted | If the patient experienced an |
| tiragolumab | prior to the tiragolumab infusion. | IRR during any previous infusion |
| | of tiragolumab, premedication | with an antihistamine and/or |
| | Vital signs (pulse rate, | antipyretic may b administered |
| | respiratory rate, blood | for subsequent doses, at the |
| | pressure, and temperature) are | discretion of the investigator. |
| | recorded within 60 minutes | Vital signs are recorded within |
| | prior to the infusion. | 60 minutes prior to the |
| | Tiragolumab is infused | tiragolumab infusion. |
| | over 60 (±15) minutes. | Tiragolumab is infused over |
| | Vital signs are recorded | 30 (±10) minutes if the previous |
| | every 15 (±5) minutes | infusion was tolerated without |
| | during the infusion. | an infusion-related reaction, or |
| | | 60 (±15) minutes if the patient |
| | | experienced an infusion- |
| | | related reaction with the |
| | | previous infusion. |
| | | Vital signs are recorded during |
| | | the infusion if clinically indicated. |
| Observation | After the infusion of | If the patient tolerated the |
| period after infusion | tiragolumab, the patient begins | previous infusion of tiragolumab |
| of tiragolumab | a 60-minute observation period. | well without infusion-associated |
| | Vital signs are recorded | adverse events, the observation |
| | at 30 (±10) minutes after the | period may be reduced |
| | infusion of tiragolumab. | to 30 minutes. |
| | Patients are informed about | If the patient experienced an |
| | the possibility of delayed post- | infusion-associated adverse |
| | infusion symptoms and are | event in the previous infusion, |
| | instructed to contact their study | the observation period should |
| | physician if they | be 60 minutes. |
| | develop such symptoms. | If clinically indicated, vital signs |
| | | should be recorded at 30 (±10) |
| | | minutes after the infusion |
| | | of tiragolumab. |
| | | Patients are informed about |
| | | the possibility of delayed post- |
| | | infusion symptoms and are |
| | | instructed to contact their study |
| | | physician if they |
| | | develop such symptoms. |

IRR = infusion-related reaction

Atezolizumab and Tiragolumab Dose Delays

The following rules apply as long as neither atezolizumab nor tiragolumab has been permanently discontinued:
Treatment cycles normally begin with dosing of atezolizumab and tiragolumab on Day 1 of each 21-day cycle. If either study drug is delayed for a related toxicity, it is recommended that the other study drug is also delayed since the safety profiles for atezolizumab and tiragolumab are similar.
In case of delays in dosing of one study drug for drug-related toxicity while the other study drug is given as planned, it is recommended that the study drug being delayed is administered at the next scheduled infusion (i.e., at the next scheduled 21-day cycle).

Chemotherapy

Sites should adhere to the information below and to local prescribing information. In general, sites should also follow their institutional and local SOC for determining dose adjustments in the event of patient weight changes. If a treatment cycle is delayed or interrupted because of toxicity resulting from either component of the chemotherapy regimen, both chemotherapy components should be held and if resumed, both should be resumed to remain synchronized.

Paclitaxel

Patients of Asian race/ethnicity receive a lower starting dose of paclitaxel at 175 mg/m² IV over 3 hours. The lower starting dose of paclitaxel for patients of Asian race/ethnicity is based on a higher overall incidence of hematologic toxicities in patients from Asian countries compared with those from non-Asian countries, which was observed during the safety review of the IMpower150 and IMpower131 study results by the independent Data Monitoring Committee. The term “Asian race/ethnicity” refers to a pan-ethnic/racial group that includes diverse populations who either live in or have ancestral origins in East Asia, Southeast Asia, or South Asia. The applicability of this term in a particular patient is at the discretion of the treating investigator and should be based on the patient's clinical characteristics and country of origin.

Paclitaxel injection must be diluted prior to infusion. Paclitaxel should be diluted in 0.9% Sodium Chloride USP; 5% Dextrose Injection, USP; 5% Dextrose and 0.9% Sodium Chloride Injection, USP; or 5% Dextrose in Ringer's Injection to a final concentration of 0.3 to 1.2 mg/mL. The infusion site should be closely monitored for possible infiltration during drug administration.

Contact of the undiluted concentrate with plasticized polyvinyl chloride (PVC) equipment or devices used to prepare solutions for infusion is not recommended. Paclitaxel should be administered through an in-line filter with a microporous membrane not greater than 0.22 μm. Use of filter devices such as IVEX-2® filters, which incorporate short inlet and outlet PVC-coated tubing, has not resulted in significant leaching of bis(2-ethylhexyl)phthalate.

Sites should follow their institutional SOC guidelines for determining the paclitaxel dose adjustments in the event of patient weight changes. For paclitaxel infusion, exceptions to the infusion time of 3 hours are allowed for sites that have an institutional policy of infusing paclitaxel more quickly (over 90 minutes) or more slowly (up to 4 hours for the first infusion).

Pemetrexed

Institutions should follow their standard administration procedures for pemetrexed. Administration of pemetrexed should be administered by IV infusion over at least 10 minutes. The premedication doses administered should be in compliance with the prescribing information. All patients eligible for pemetrexed therapy should avoid taking non-steroidal anti-inflammatory drugs with long drug-elimination half-lives for at least 5 days prior to, on the day of, and at least 2 days following pemetrexed administration.

Gemcitabine

Gemcitabine should be administered by IV infusion over 30 minutes prior to carboplatin or cisplatin. Note that the dose of gemcitabine is different when administered before carboplatin versus when administered before cisplatin (Table 57). Gemcitabine must be diluted prior to infusion. The recommended diluent for reconstitution of gemcitabine is 0.9% Sodium Chloride Injection, USP, without preservatives. The administration of gemcitabine should be done in accordance with local practice and the prescribing information.

Carboplatin

Carboplatin should be administered by IV infusion, immediately after the completion of paclitaxel or pemetrexed administration, over 15-30 minutes to achieve an initial target area under the concentration-time curve (AUC) of 6 mg/mL/min (Calvert formula dosing) and with standard anti-emetic medications per local practice guidelines.

The carboplatin dose of AUC 6 mg/mL/min is calculated using the Calvert formula (Calvert et al., J Clin Oncol, 7: 1748-1756, 1989):

Total dose (mg)=(target AUC)×(glomerular filtration rate [GFR]+25)

The GFR used in the Calvert formula to calculate AUC-based dosing should not exceed 125 mL/min. For the purposes of this study, the GFR is considered to be equivalent to the creatinine clearance (CrCl). The CrCl is calculated by institutional guidelines or by the method of Cockcroft and Gault (Nephron, 16: 31-41, (1976)) using the following formula:

CrCl=((140−age) (wt)/(72×Scr))×0.85 if female

Where: CrCl=creatinine clearance in mL/min; age=patient's age in years; wt=patient's weight in kg; Scr=serum creatinine in mg/dL.

For patients with an abnormally low serum creatinine level, GFR should be estimated using a minimum creatinine level of 0.8 mg/dL or cap the estimated GFR at 125 mL/min. If a patient's GFR is estimated based on serum creatinine measurements by the isotope dilution mass spectroscopy method, the FDA recommends that physicians consider capping the dose of carboplatin for desired exposure (AUC) to avoid potential toxicity due to overdosing. Based on the Calvert formula described in the carboplatin label, the maximum doses can be calculated as follows:

Maximum carboplatin dose (mg)=target AUC (mg·min/mL) (GFR×25 mL/min)

The maximum dose is based on a GFR estimate that is capped at 125 mL/min for patients with normal renal function. No higher estimated GFR values should be used. For a target AUC=6, the maximum dose is 6×(125+25)=900 mg. For a target AUC=5, the maximum dose is 5×(125+25)=750 mg. For a target AUC=4, the maximum dose is 4×(125+25)=600 mg.

Cisplatin

Cisplatin is administered by IV infusion, approximately 30 minutes after completion of the pemetrexed or gemcitabine infusion at a dose of 75 mg/m² over 1-2 hours or per SOC at the institution. Patients must receive adequate antiemetic treatment and appropriate hydration prior to and after receiving cisplatin.

E. Surgical Treatment Plan

An attending thoracic surgeon with experience in early-stage resectable NSCLC evaluates patients at screening to determine surgical fitness and eligibility for surgical resection. Patients must be eligible for an R0 resection with curative intent at time of screening. The intent to downstage in order to render patient operable is not permitted.

At screening, patients must be confirmed for surgical fitness based on their pulmonary function tests (PFTs) as outlined in the inclusion criteria. ppoFEV1 and ppoDL_{CO} are calculated using the following methodology (Brunelli et al., Chest, 143(5 Suppl): e166S-e190S, 2013):
Perfusion method (if pulmonary ventilation/perfusion scan (VQ scan) is performed:
   ppoFEV₁ or ppoDL_{CO}=preoperative FEV₁ or DL_{CO}×(1−x)
   Where: x=fraction of total perfusion for the resected lung
Anatomic method:
   ppoFEV₁ or ppoDL_{CO}=preoperative FEV₁ or DL_{CO}×(1−(y/z))
   Where: y=number of functional or unobstructed lung segments to be removed; z=19, total number of functional lung segments. Note that the total number of functional lung segments (19) is represented by 10 segments in the right lung (3 in the upper lobe, 2 in the middle lobe and 5 in the lower lobe) and 9 segments in the left lung (5 in the upper lobe, and 4 in the lower lobe).

Patients are reassessed after completion of neoadjuvant treatment and prior to surgery by the attending surgeon and medical oncologist. Preoperative evaluation, including, but not limited to, blood tests, coagulation, cardiac tests or PFTs (if indicated), anesthesia assessment, and other evaluation procedures, should be performed per local SOC.

The surgical procedure should be performed within 30 days after the pre-surgery visit as best as possible. If surgery cannot be performed within this time window (e.g., because of a prolonged adverse event), the Medical Monitor should be consulted. If surgery is planned beyond 30 days after the pre-surgery visit a repeat CT scan should be obtained prior to the planned surgery.

Resection should be accomplished via an open or minimally invasive procedure (e.g., thoracotomy, sternotomy, clamshell or hemiclamshell incision, or video-assisted thoracic surgery [VATS] or robotically assisted VATS).

Pathological complete resection of the primary tumor, and involved lymph nodes (R0) should be performed. Anatomic resection by means of segmentectomy, lobectomy, bilobectomy, or pneumonectomy is required. For all resections, the division of all anatomic structures should be included (e.g., artery, bronchus, vein). Wedge resections are not allowed. Deep wedge resections that include a segment (e.g., apical or superior segment wedge resection) that do not provide the aforementioned anatomic details do not count as a segmentectomy.

Hilar and mediastinal lymph node dissection or sampling is mandatory. For right-sided resections, this involves at least lymph nodes from levels 4R, 7, 10R, and 11R.

Inclusion of level 2R nodes is encouraged. For left-sided resections, this involves at least lymph nodes from levels 5/6, 7, 10L, and 11 L. For lower-lobe resection, including levels 8 or 9 is encouraged.

F. Post-Operative Radiotherapy

Post-operative radiotherapy (PORT) is allowed for patients with confirmed, pathological N2+disease (ypN2), or positive tumor margins (R1 or R2) present at the time of surgical resection and must be administered prior to adjuvant Atezo+Tira treatment or after adjuvant platinum-based chemotherapy.

Conformal 3-dimensional or intensity-modulated radiation therapy to a recommended dose of 50-66 grays (Gy) in 1.8-2 Gy fractions should be administered in accordance with institutional and/or international guidelines. Strongly recommended constraints include a mean dose to the lung 20 Gy and a V20 (percentage of the lung volume that receives radiation doses of 20 Gy or more) not to exceed 31% after lobectomy, or a mean lung dose of 8Gy and a V20 5% after pneumonectomy.

G. Concomitant Therapy

Concomitant therapy consists of any medication (e.g., prescription drugs, over-the counter drugs, vaccines, herbal or homeopathic remedies, nutritional supplements) used by a patient in addition to protocol-mandated treatment from 7 days prior to initiation of study drug to the treatment discontinuation visit.

Permitted Therapy

Patients are permitted to use the following therapies during the study:
Oral contraceptives with a failure rate of <1% per year.
Hormone-replacement therapy.
Prophylactic or therapeutic anticoagulation therapy (such as warfarin at a stable dose or low-molecular-weight heparin).
Inactivated influenza vaccinations.
Megestrol acetate administered as an appetite stimulant.
Mineralocorticoids (e.g., fludrocortisone).
Corticosteroids administered for COPD or asthma.
Low-dose corticosteroids administered for orthostatic hypotension or adrenocortical insufficiency.

Premedication with antihistamines, antipyretics, and/or analgesics may be administered for the second and subsequent atezolizumab infusions only, at the discretion of the investigator.

In general, investigators should manage a patient's care (including preexisting conditions) with supportive therapies other than those defined as cautionary or prohibited therapies as clinically indicated, per local standard practice.

Patients who experience infusion-associated symptoms may be treated symptomatically with acetaminophen, ibuprofen, diphenhydramine, and/or H2-receptor antagonists (e.g., famotidine, cimetidine), or equivalent medications per local standard practice. Serious infusion-associated events manifested by dyspnea, hypotension, wheezing, bronchospasm, tachycardia, reduced oxygen saturation, or respiratory distress should be managed with supportive therapies as clinically indicated (e.g., supplemental oxygen and β2-adrenergic agonists.

Cautionary Therapy for Atezolizumab- and Tiragolumab-Treated Patients

Corticosteroids and TNF-α inhibitors

Systemic corticosteroids and TNF-α inhibitors may attenuate potential beneficial immunologic effects of treatment with atezolizumab. Therefore, in situations in which systemic corticosteroids or TNF-α inhibitors would be routinely administered, alternatives, including antihistamines, should be considered. If the alternatives are not feasible, systemic corticosteroids and TNF-α inhibitors may be administered at the discretion of the investigator.

Systemic corticosteroids are recommended, at the discretion of the investigator, for the treatment of specific adverse events when associated with atezolizumab and/or tiragolumab therapy.

Prohibited Therapy

Use of the following concomitant therapies is prohibited as described below:
Concomitant therapy intended for the treatment of cancer (including, but not limited to, chemotherapy, hormonal therapy, immunotherapy, radiotherapy, and herbal therapy), whether health authority-approved or experimental, is prohibited for various time periods prior to starting study treatment, depending on the agent, and during study treatment, until disease progression is documented and the patient has discontinued study treatment.
Investigational therapy within 42 days prior to initiation of study treatment and during study treatment.
Live, attenuated vaccines (e.g., FLUMIST®) are prohibited within 4 weeks prior to initiation of study treatment, during study treatment, for 90 days after the final dose of tiragolumab, and for 5 months after the final dose of atezolizumab. Patients being treated with chemotherapy (i.e., carboplatin, cisplatin, pemetrexed, paclitaxel, or gemcitabine) should not receive live vaccines.
Systemic immunostimulatory agents (including, but not limited to, interferons and IL-2) are prohibited within 4 weeks or 5 drug-elimination half-lives (whichever is longer) prior to initiation of study treatment and during study treatment because these agents could potentially increase the risk for autoimmune conditions when given in combination with atezolizumab and tiragolumab.
Systemic immunosuppressive medications (including, but not limited to, cyclophosphamide, azathioprine, methotrexate, and thalidomide) during study treatment because these agents could potentially alter the efficacy and safety of study treatments.

H. Study Assessments

Patients are closely monitored for safety and tolerability throughout the study. Patients should be assessed for toxicity prior to each dose; dosing occurs only if the clinical assessment and local laboratory test values are acceptable.

All treatment visits must occur ±3 days from the scheduled date unless otherwise noted. All assessments should be performed on the day of the specified visit unless a time window is specified. Patients must be assessed for toxicity prior to each dose; dosing occurs only if the clinical assessment and local laboratory test values are acceptable. Assessments scheduled on the day of study treatment administration should be performed prior to dosing, unless otherwise specified.

The following assessments may be performed 4 days before Day 1 of each cycle:
Limited physical examination
Local laboratory tests

With the exception of tumor assessments, screening assessments performed 4 days before Day 1 of Cycle 1 are not required to be repeated on Day 1 of Cycle 1.

If a holiday, weekend, or other event precludes scheduled dosing, dosing may be postponed to the soonest following date, with subsequent dosing continuing on a 21-day schedule. If treatment is postponed for fewer than 3 days, the patient can resume the original schedule.

I. Tumor Response and Disease Status Follow-Up Evaluations

After surgery and during the adjuvant treatment phase, disease status follow-up assessments are performed in all patients (both cohorts) every 4 months from the day of surgery by chest CT with IV contrast (including the liver and adrenal glands) for the first year and then every 6 months in the second year. If a CT scan with contrast is contraindicated (e.g., in patients with impaired renal clearance), a non-contrast CT scan of the chest may be performed. Patients who have not experienced recurrence of Disease undergo disease status follow-up assessments every 6 months by chest CT scan with IV contrast (including liver and adrenals) during Years 3-5 post-surgery.

Disease status follow-up assessments should occur within the allowed time-window of the scheduled follow-up evaluation. The allowed time window for disease status follow-up assessments are ±7 days in Year 1, 14 days in Years 2-3, and ±4 weeks thereafter.

Disease recurrence after surgery should be confirmed pathologically and/or by unequivocal radiographic evidence. If a scan shows equivocal findings (e.g., mediastinal nodes measuring <1.5 cm in the short axis, lung parenchymal lesions or visceral lesions measuring <1 cm in the longest diameter), a biopsy should be performed. If a biopsy is not feasible or safe, then confirmatory scans should be performed again within 4 to 8 weeks. If the confirmatory scan confirms disease recurrence, then the event should be documented at the previous confirmatory scan date that showed the equivocal event. The biopsy should be performed prior to starting next anti-cancer therapy. If the biopsy does not show evidence of disease recurrence (e.g., non-malignant infiltrates), then the patient may continue with scheduled study treatment, assessments, and/or follow-up.

Tumor or disease status follow-up assessments should continue in patients who discontinue treatment early for reasons other than disease progression or recurrence (e.g., because of toxicity). In the absence of disease progression or recurrence, tumor or disease status follow-up assessments should continue in all patients, regardless of whether they start new anti-cancer therapy, until disease progression or recurrence, withdrawal of consent, death, loss to follow-up, or study termination by the Sponsor, whichever occurs first.

J. Study Treatment Discontinuation

Patients must permanently discontinue study treatment if they experience any of the following:
Intolerable toxicity related to atezolizumab or tiragolumab, including development of an immune-mediated adverse event determined by the investigator to be unacceptable given the individual patient's potential response to therapy and severity of the event. Chemotherapy may be continued if atezolizumab and tiragolumab is discontinued during the neoadjuvant treatment phase.
Intolerable toxicity related to other components of the study treatment. If any or all chemotherapeutic agents have been discontinued because of chemotherapy-related toxicities during the neoadjuvant treatment phase, Atezo+Tira should still be administered.
Any medical condition that may jeopardize the patient's safety if he or she continues study treatment.
Investigator or Sponsor determination that treatment discontinuation is in the best interest of the patient.
Use of another non-protocol-specified anti-cancer therapy.
Pregnancy.
Unequivocal radiographic disease progression per RECIST v1.1 during the neoadjuvant treatment phase.
Pathologically confirmed or unequivocal radiographic evidence of disease recurrence during the adjuvant treatment phase.

Patients who receive at least 2 cycles of neoadjuvant treatment and discontinue study treatment prematurely are not replaced. Patients who receive fewer than two cycles of neoadjuvant treatment may be replaced if the reason for early treatment discontinuation is not an adverse event of special interest or disease progression.

Patients who discontinue neoadjuvant study treatment prior to receiving four cycles (e.g. because of treatment intolerability or lack of response) are assessed by the treating medical oncologist and attending surgeon for surgical resection or non-surgical standard-of-care treatment measures (if not amenable for surgery). Patients who proceed with planned protocol-specified surgery remain eligible for all post-operative study treatment and procedures.

Patients return to the clinic for a treatment discontinuation visit 30 days after the final dose of study treatment. The visit at which the disease assessment shows progressive disease or confirms disease recurrence may be used as the treatment discontinuation visit. Patients who discontinue study treatment for any reason other than progressive disease or recurrence continue to undergo tumor or disease status assessments.

### Example 11. Assessment of Safety for GO042501 Study

A. Safety Plan

The safety plan for patients in this study is based on anticipated mechanism of action, results from nonclinical studies, published data on similar molecules, clinical experience with tiragolumab alone and in combination with atezolizumab in Phase I and II studies, and the clinical safety profile of atezolizumab.

Measures are taken to ensure the safety of patients participating in this study, including the use of stringent inclusion and exclusion criteria and close monitoring of patients during the study. Administration of atezolizumab and tiragolumab is performed in a monitored setting in which there is immediate access to trained personnel and adequate equipment and medicine to manage potentially serious reactions.

B. Management of Adverse Events

Dose Modifications

There are no dose modifications, including dose reductions, for atezolizumab or tiragolumab in the study.

Treatment Interruptions

Study treatment may be temporarily suspended as appropriate for management of toxicity. On the basis of the available characterization of mechanism of action, tiragolumab may cause adverse events similar to but independent of atezolizumab, may exacerbate the frequency or severity of atezolizumab-related adverse events, or may have non-overlapping toxicities with atezolizumab. Because these scenarios may not be distinguished from one another in the clinical setting, immune-mediated adverse events should generally be attributed to both study drugs, and dose interruptions or treatment discontinuation in response to immune-mediated adverse events should be applied to both atezolizumab and tiragolumab.

In the adjuvant setting atezolizumab and tiragolumab may be held for a maximum of 12 weeks. If tiragolumab is interrupted for >12 weeks for any reason, the patient must permanently discontinue tiragolumab treatment, but may continue atezolizumab if there is no contraindication and after discussion to determine whether the toxicity is considered related to tiragolumab and/or to the combination. An exception can be made if in the judgment of the investigator, the patient is likely to derive clinical benefit from resuming tiragolumab after a hold of >12 weeks. In this case, tiragolumab may be restarted. If atezolizumab is interrupted for >12 weeks, the patient must permanently discontinue atezolizumab. However, if, in the judgment of the investigator, the patient is likely to derive clinical benefit from atezolizumab after a hold of >12 weeks, atezolizumab may be restarted. Continued dosing of patients with single-agent atezolizumab requires that all other study eligibility criteria continue to be met.

Continued administration of single-agent tiragolumab after permanent discontinuation of atezolizumab is not permitted.

If a patient must be tapered off steroids used to treat adverse events, atezolizumab and/or tiragolumab may be withheld for additional time beyond >12 weeks from the last dose, and tiragolumab may be withheld for an additional time beyond >12 weeks from the last dose until steroids are discontinued, or until steroids are reduced to prednisone dose (or dose equivalent) 10 mg/day. Dose interruptions for reason(s) other than toxicity may be allowed. After both study treatments have been discontinued, the patient is monitored for safety and efficacy.

C. Safety Parameters and Definitions

Safety assessments consist of monitoring and recording adverse events, including serious adverse events and adverse events of special interest, performing protocol-specified safety laboratory assessments, measuring protocol-specified vital signs, and conducting other protocol-specified tests that are deemed critical to the safety evaluation of the study.

Adverse Events

According to the ICH guideline for Good Clinical Practice, an adverse event is any untoward medical occurrence in a clinical investigation subject administered a pharmaceutical product, regardless of causal attribution. An adverse event can therefore be any of the following:
Any unfavorable and unintended sign (including an abnormal laboratory finding), symptom, or disease temporally associated with the use of a medicinal product, whether or not considered related to the medicinal product.
Any new disease or exacerbation of an existing disease (a worsening in the character, frequency, or severity of a known condition).
Recurrence of an intermittent medical condition (e.g., headache) not present at baseline.
Any deterioration in a laboratory value or other clinical test (e.g., ECG, X-ray) that is associated with symptoms or leads to a change in study treatment or concomitant treatment or discontinuation from study treatment.
Adverse events that are related to a protocol-mandated intervention, including those that occur prior to assignment of study treatment (e.g., screening invasive procedures such as biopsies).

Serious Adverse Events

A serious adverse event is any adverse event that meets any of the following criteria:
Is fatal (i.e., the adverse event actually causes or leads to death).
Is life threatening (i.e., the adverse event, in the view of the investigator, places the patient at immediate risk of death). This does not include any adverse event that, had it occurred in a more severe form or was allowed to continue, might have caused death.
Requires or prolongs inpatient hospitalization.
Results in persistent or significant disability/incapacity (i.e., the adverse event results in substantial disruption of the patient's ability to conduct normal life functions).
Is a congenital anomaly/birth defect in a neonate/infant born to a mother exposed to study treatment.
Is a significant medical event in the investigator's judgment (e.g., may jeopardize the patient or may require medical/surgical intervention to prevent one of the outcomes listed above).

The terms “severe” and “serious” are not synonymous. Severity refers to the intensity of an adverse event (e.g., rated as mild, moderate, or severe, or according to NCI CTCAE); the event itself may be of relatively minor medical significance (such as severe headache without any further findings).

Adverse Events of Special Interest

Adverse events of special interest for this study are as follows:
Cases of potential drug-induced liver injury that include an elevated ALT or AST in combination with either an elevated bilirubin or clinical jaundice, as defined by Hy's Law.
Suspected transmission of an infectious agent by the study treatment, as defined below:
   Any organism, virus, or infectious particle (e.g., prion protein transmitting transmissible spongiform encephalopathy), pathogenic or non-pathogenic, is considered an infectious agent. A transmission of an infectious agent may be suspected from clinical symptoms or laboratory findings that indicate an infection in a patient exposed to a medicinal product. This term applies only when a contamination of study treatment is suspected.
Pneumonitis.
Colitis.
Endocrinopathies: diabetes mellitus, pancreatitis, adrenal insufficiency, hyperthyroidism, and hypophysitis.
Hepatitis, including AST or ALT >10×ULN.
Systemic lupus erythematosus.
Neurological disorders: Guillain-Barré syndrome, myasthenic syndrome or myasthenia gravis, and meningoencephalitis.
Events suggestive of hypersensitivity, infusion-related reactions, cytokine release syndrome, hemophagocytic lymphohistiocytosis, and macrophage activation syndrome.
Nephritis.
Ocular toxicities (e.g., uveitis, retinitis, optic neuritis).
Myositis.
Myopathies, including rhabdomyolysis.
Grade ≥2 cardiac disorders (e.g., atrial fibrillation, myocarditis, pericarditis).
Vasculitis.
Autoimmune hemolytic anemia.
Severe cutaneous reactions (e.g., Stevens-Johnson syndrome, dermatitis bullous, toxic epidermal necrolysis).

Assessment of Severity of Adverse Events

The adverse event severity grading scale for the NCI CTCAE (v5.0) is used for assessing adverse event severity. The American Society for Transplantation and Cellular Therapy (ASTCT) CRS Consensus Grading Scale should be used in addition to NCI CTCAE v5.0 when reporting the severity of CRS.

### Example 12. Statistical considerations and analysis plan for GO42501 study

Efficacy and safety analyses are performed on all patients enrolled in the GO42501 study who have received at least one dose of the study drug, for each cohort.

Determination of Sample Size

The primary efficacy objective of the study is to evaluate major pathological response (MPR) rate. Approximately 41 patients are enrolled in each cohort in the study. Assuming an observed MPR rate of 61%, this sample size provides adequate precision for the point estimate for the MPR rate in each cohort with the lower bound of the two-sided 95% CI exceeding 46%, a rate that may warrant further investigation of the combination therapy in this setting.

Summaries of Conduct of Study

The number of patients who enroll, discontinue, or complete the study is summarized. Reasons for premature study withdrawal are listed and summarized. Enrollment and major protocol deviations are listed for each treatment cohort.

A. Summaries of Demographic and Baseline Characteristics

Demographic information such as age and race are tabulated. Descriptive statistics, including means, standard deviations, and ranges for continuous parameters, as well as percentages and frequencies for categorical parameters, are presented. Summaries are presented for overall population and for each treatment cohort. Baseline measurements are the last available data obtained prior to the patient receiving the first dose of any component of study drug, unless otherwise noted. B. Safety Analyses

For each cohort, the safety analyses include all enrolled patients who receive at least one dose of study treatment.

Study treatment exposure is summarized, including treatment duration, dosage, and dose intensity.

Incidence and length of surgical delays, incidence of operative and post-operative complications and/or number of surgical cancellations related to study treatment are evaluated for each cohort.

Verbatim description of adverse events are mapped to the MedDRA thesaurus terms. Severity for all adverse events are graded by the investigator according to the NCI CTCAE v5.0, and severity for CRS is also graded by the investigator according to the ASTCT Consensus Grading Scale. These summaries are presented by treatment arm. All adverse events are summarized by treatment arm and NCI CTCAE grade. CRS is also summarized by treatment arm and ASTCT Consensus grade. In addition, the treatment-emergent adverse events leading to withdrawal of study treatment, leading to dose reduction or interruption, related to study treatment, severe (i.e., Grade ≥3 adverse events), fatal adverse events (i.e., Grade 5), and serious adverse events, and adverse events of special interest are also summarized. Multiple occurrences of the same event are counted once at the maximum severity. Laboratory data with values outside of the normal ranges are identified. Additionally, selected laboratory data, including ADA results, and changes in vital signs are summarized by treatment arm. Deaths and causes of deaths are summarized.

C. Efficacy Analyses

The efficacy analysis population includes all enrolled patients who received at least one dose of the study treatment.

Primary Efficacy Endpoint

MPR rate is defined as the proportion of patients who have achieved MPR and is estimated for each treatment cohort in the efficacy analysis population. MPR is defined as ≤10% residual viable tumor at the time of surgical resection in the primary tumor, as assessed by central pathology laboratory. Patients who do not proceed to surgery are considered as non-responders for MPR. The two-sided 95% CI for MPR rate calculated using the Clopper-Pearson method is reported.

Secondary Efficacy Endpoints

Pathological complete response (pCR) rate is defined as the proportion of patients who have achieved pCR. pCR is defined as the absence of any viable primary tumor at the time of surgical resection, as assessed by central pathology laboratory. pCR rate is analyzed using the same statistical methodology as MPR rate for each treatment cohort for the efficacy analysis population.

Event-free survival (EFS) is defined as the time from first dose of the study drug to any of the following events, whichever occurs first: disease progression that precludes surgery, as assessed by the investigator according to RECIST v1.1; local or distant disease recurrence (including occurrence of new primary NSCLC); or death from any cause. Patients who have not experienced disease progression that precludes surgery, local or distant disease recurrence, or died at the time of analysis are censored at the time of last tumor or disease follow-up assessment. Patients with no post-baseline tumor assessment are censored at the date of first dose of the study drug. EFS is evaluated using the Kaplan-Meier method for each treatment cohort for the efficacy analysis population.

D. Pharmacokinetic Analyses

Samples are collected for PK analyses and to compare exposure in this study with that attained in previous studies. Serum concentrations of atezolizumab and tiragolumab are reported as individual values and summarized (mean, standard deviation, coefficient of variation, median, range, geometric mean, and geometric mean coefficient of variation) by cohort and cycle, when appropriate and as data allow.

Individual and median serum concentrations of atezolizumab and tiragolumab are plotted by cohort and day. Atezolizumab and tiragolumab concentration data may be pooled with data from other studies using an established population-PK model to derive PK parameters such as clearance, volume of distribution, and AUC, as warranted by the data. Potential correlations of relevant PK parameters with safety, efficacy, or biomarker outcomes may be explored.

E. Immunogenicity Analyses

The immunogenicity analyses include patients with any anti-drug antibody (ADA) assessments, with patients grouped according to treatment received. The numbers and proportions of treatment-emergent ADA-positive patients and ADA-negative patients for both atezolizumab and tiragolumab are summarized by cohort. The relationship between ADA status and safety, efficacy, and PK endpoints may be analyzed and reported via descriptive statistics.

F. Biomarker Analyses

Exploratory biomarker analyses are performed in order to explore the relationship between multiple pathological, immunological, and genomic characteristics of the cancers and clinical outcome in these patients. These analyses include, but are not limited to, characterization of immune cells and ctDNA in the tumor microenvironment and/or periphery. Clinical efficacy outcomes including MPR, pCR, and EFS are analyzed in the biomarker subgroups (e.g., based on PD-L1 and TIGIT status) to evaluate the treatment benefit if feasible.

### Example 13. Efficacy of an Anti-TIGIT Antagonist Antibody in Combination with a PD-1 Axis Binding Antagonist in Patients with Cervical Cancer

The efficacy and safety of treatment with an anti-TIGIT antagonist antibody (e.g., an anti-TIGIT antibody disclosed herein, e.g., tiragolumab) in combination with a PD-1 axis binding antagonist (e.g., an anti-PD-L1 antagonist antibody (e.g., atezolizumab)) and of atezolizumab monotherapy in patients with metastatic and/or recurrent PD-L1-positive (tumor cells and tumor-associated immune cells [TICs]≥5%) cervical cancer after progression or recurrence from at least one platinum-based but no more than two prior systemic therapies is evaluated. To be eligible, patients must have an Eastern Cooperative Oncology Group (ECOG) Performance Status of 0 or 1 and metastatic and/or recurrent PD-L1-positive cervical cancer.

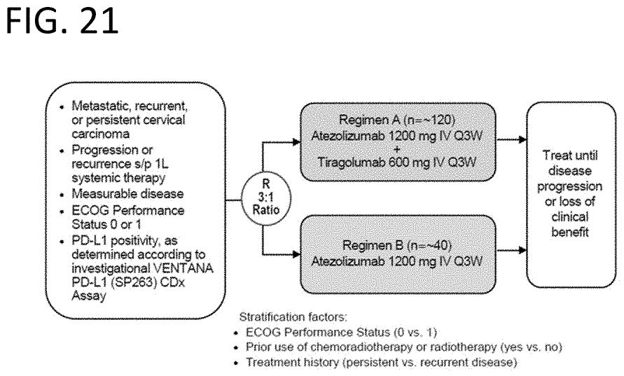

The clinical trial consists of a single phase, as described in detail below and illustrated in FIG. 21.

Randomization

In this study, approximately 160 patients are enrolled and assigned to one of two treatment arms of tiragolumab in combination with atezolizumab or atezolizumab monotherapy. Randomization occurs in a 3:1 ratio through use of a permuted-block randomization method to ensure a balanced assignment to each treatment arm. Randomization is stratified according to ECOG Performance Status (0 vs. 1), prior use of chemoradiotherapy or radiotherapy (yes vs. no), and treatment history (persistent versus recurrent disease).

Eligibility

The study enrolls female patients with a minimum age of 18 years according to the following inclusion criteria:
Histologically confirmed recurrent or persistent squamous cell carcinoma, adenosquamous carcinoma, or adenocarcinoma of the cervix (neuroendocrine, clear cell, and sarcoma histologies are not allowed) after 1-2 lines of prior systemic chemotherapy that is not amenable to curative treatment with systemic chemotherapy, surgery, and/or radiotherapy.
   At least one prior line of platinum-based systemic chemotherapy. Radiosensitizing cisplatin given with radiotherapy is not considered a line of systemic chemotherapy.
Radiologically-measurable disease
Eastern Cooperative Oncology Group (ECOG) performance Status of 0 or 1
Cervical cancer tissue for study analysis (archival or fresh biopsy specimen)
Life expectancy of at least 12 weeks
Adequate hematologic and organ function
Female of childbearing potential must be willing to comply with adequate contraception

Patients are excluded from enrollment based on the following criteria:
Treatment with investigational therapy with therapeutic intent within 28 days prior to randomization and during study treatment
Any central nervous system (CNS) or brain metastases
Active or history of autoimmune disease or immune deficiency
Active tuberculosis
Known, clinically significant liver disease
Severe infection per investigator judgement at the time of randomization
Prior treatment with CD137 agonists or immune checkpoint blockade therapies, anti-CTLA-4, anti-TIGIT, anti-PD-1, and anti-PD-L1 therapeutic antibodies
Treatment with systemic immunostimulatory agents within 4 weeks or 5 drug-elimination half-lives (whichever is longer) prior to randomization
Treatment with systemic immunosuppressive medications within 1 week prior to randomization or anticipation of need for systemic immunosuppressive medication during study
Pregnant or breastfeeding woman.
Known hypersensitivity to any component of the tiragolumab or atezolizumab formulations

A. Study Treatment Dosage and Administration

During treatment, patients receive a fixed dose of 1200 mg of atezolizumab or a fixed dose of 1200 mg of atezolizumab and a fixed dose of 600 mg of tiragolumab. Atezolizumab is administered at a fixed dose of 1200 mg Q3W (1200 mg on Day 1 of each 21-day cycle). Tiragolumab in combination with atezolizumab is administered to all patients in the experimental arm at a fixed dose of 600 mg IV Q3W on Day 1 of each 21-day cycle.

In an alternative trial, on the days of administration, atezolizumab is administered as a monotherapy as per the instructions outlined in Table 59. Prior to the first infusion, the patient's vital signs (e.g., pulse rate, respiratory rate, blood pressure, and temperature) are recorded within 60 minutes before starting the infusion. The first infusion of atezolizumab is administered over 60 (±15) minutes while the patient's vital signs are recorded every 15 (±5) minutes during the infusion and at 30 (±10) minutes after the infusion. If no infusion-associated adverse events are experienced during the first infusion, subsequent infusions can be administered over 30 (±10) minutes. Pre-infusion recordation of vital signs shall continue to be recorded within 60 minutes prior to the start of infusion of atezolizumab.

TABLE 59
Administration of First and Subsequent Atezolizumab Monotherapy Infusions

| First Infusion | Subsequent Infusions |
|---|---|
| No premedication is permitted prior | If the patient experienced an infusion-related |
| to the atezolizumab infusion. | reaction (IRR) with any previous infusion, |
| Vital signs (pulse rate, respiratory rate, blood | premedication with antihistamines, antipyretics, |
| pressure, and temperature) should be measured | and/or analgesics may be administered |
| within 60 minutes prior to the infusion. | for subsequent doses at the |
| Atezolizumab should be infused | discretion of the investigator. |
| over 60 (±10) minutes. | Vital signs should be measured within 60 |
| If clinically indicated, vital signs should be | minutes prior to the infusion. |
| measured every 15 (±5) minutes during the | Atezolizumab should be infused over 30 (±10) |
| infusion and at 30 (±10) minutes | minutes if the previous infusion was tolerated |
| after the infusion. | without an IRR, or 60 (±15) minutes if the patient |
| Patients should be informed about | experienced an IRR with the previous infusion. |
| the possibility of delayed post- | If the patient experienced an IRR with the |
| infusion symptoms and instructed | previous infusion or if clinically indicated, vital |
| to contact their study physician if | signs should be measured during the infusion and |
| they develop such symptoms. | at 30 (±10) minutes after the infusion. |

In an alternative trial, on the days of administration, atezolizumab is administered prior to tiragolumab as per the instructions outlined in Table 60. Prior to the first infusion, the patient's vital signs (e.g., pulse rate, respiratory rate, blood pressure, and temperature) are recorded within 60 minutes before starting the infusion. The first infusion of atezolizumab is administered over 60 (±15) minutes while the patient's vital signs are recorded every 15 (±5) minutes during the infusion and at 30 (±10) minutes after the infusion. The first infusion of the anti-TIGIT antibody (e.g., an anti-TIGIT antibody disclosed herein, e.g., tiragolumab) is administered over 60 (±10) minutes while the patient's vital signs are recorded every 15 (±5) minutes during the infusion and at 30 (±10) minutes after the infusion. If no infusion-associated adverse events are experienced during the first infusion, subsequent infusions can be administered over 30 (±10) minutes. Pre-infusion recordation of vital signs shall continue to be recorded within 60 minutes prior to the start of infusion of atezolizumab.

Treatment may be continued as long as patients are experiencing clinical benefit in the absence of unacceptable toxicity or symptomatic deterioration attributed to disease progression after an integrated assessment of radiographic data, biopsy results, and clinical status. Patients who meet the criteria for equivocal disease progression per independent review committee (IRC)-determined RECIST v1.1 will be permitted to continue treatment (tiragolumab in combination with atezolizumab or atezolizumab alone) if they meet all of the criteria specified.

TABLE 60
Administration of First and Subsequent Tiragolumab and Atezolizumab Infusions

| | First Infusion | Subsequent Infusions |
|---|---|---|
| Atezolizumab | No premedication is permitted for | If the patient experienced an IRR |
| infusion | the first atezolizumab infusion, | during any previous infusion of |
| | Vital signs (pulse rate, | atezolizumab, premedication with |
| | respiratory rate, blood | antihistamines, antipyretics, |
| | pressure, and temperature) | and/or analgesics may be |
| | should be recorded within 60 | administered for subsequent |
| | minutes prior to the infusion, | doses at the discretion |
| | IAtezolizumab should be | of the investigator. |
| | infused over 60 (±10) minutes. | Vital signs should be recorded |
| | If clinically indicated, vital signs | within 60 minutes prior |
| | should be measured every | to the infusion. |
| | 15 (±5) minutes during the | Atezolizumab should be infused |
| | infusion and at 30 (±10) | over 30 (±10) minutes if the |
| | minutes after the infusion. | previous infusion was tolerated |
| | | without an IRR or 60 (±15) |
| | | minutes if the patient experienced |
| | | an IRR with the previous infusion. |
| | | If the patient experienced an IRR |
| | | with the previous infusion, or if |
| | | clinically indicated, vital signs |
| | | should be recorded |
| | | during the infusion. |
| Observation | After the infusion of | If the patient tolerated the |
| period after infusion | atezolizumab, the patient begins | previous atezolizumab infusion |
| of atezolizumab | a 60-minute observation period, | well without infusion-associated |
| | Vital signs should be recorded | adverse events, the observation |
| | at 30 (±10) minutes after the | period after the next and |
| | infusion of atezolizumab. | following infusions may be |
| | Patients should be informed | reduced to 30 minutes. |
| | about the possibility of delayed | If the patient experienced |
| | post-infusion symptoms and | infusion-associated adverse |
| | instructed to contact their | events in the previous infusion, |
| | study physician if they | the observation period |
| | develop such symptoms. | should be 60 minutes. |
| | | If clinically indicated, vital |
| | | signs should be recorded |
| | | at 30 (±10) minutes |
| | | after the infusion of |
| | | atezolizumab. |
| Infusion of | No premedication is permitted | If the patient experienced an |
| tiragolumab | prior to the tiragolumab infusion. | IRR during any previous |
| | Vital signs (pulse rate, | infusion of tiragolumab, |
| | respiratory rate, blood pressure, | premedication with |
| | and temperature) should be | antihistamines, antipyretics, |
| | recorded within 60 minutes prior | and/or analgesics may be |
| | to the infusion, | administered for subsequent |
| | Tiragolumab should be | doses at the discretion |
| | infused over 60 (±10) minutes. | of the investigator. |
| | Vital signs should be recorded | Vital signs should be recorded |
| | every 15 (±5) minutes | within 60 minutes prior to the |
| | during the infusion. | tiragolumab infusion. |
| | | Tiragolumab should be infused |
| | | over 30 (±10) minutes if the |
| | | previous infusion was tolerated |
| | | without an infusion-related |
| | | reaction, or 60 (±15) minutes if |
| | | patient experienced an infusion- |
| | | related reaction with |
| | | the previous infusion. |
| | | Vital signs should be recorded |
| | | during the infusion |
| | | if clinically indicated. |
| Observation | After the infusion of tiragolumab, | If the patient experienced an |
| period after infusion | the patient begins a 60-minute | infusion-associated adverse |
| of tiragolumab | observation period, | event in the previous infusion, the |
| | Vital signs should be recorded | observation period should be 60 |
| | at 30 (±10) minutes after the | minutes. If clinically indicated, |
| | infusion of tiragolumab. | vital signs should be recorded at |
| | Patients will be informed about | 30 (±10) minutes after the |
| | the possibility of delayed post- | infusion of tiragolumab. |
| | infusion symptoms and will be | Patients will be informed |
| | instructed to contact their | about the possibility of |
| | study physician if they | delayed post-infusion |
| | develop such symptoms. | symptoms and will be |
| | | instructed to contact their |
| | | study physician if they |
| | | develop such symptoms. |

B. Concomitant Therapy

Certain concomitant therapies are permitted. Concomitant therapies include any medication (e.g., oral contraceptives, hormone-replacement therapy, prescription drugs, over the counter drugs, vaccines, herbal or homeopathic remedies, nutritional supplements) used by a patient in addition to protocol-mandated study treatment from seven days prior to initiation of study treatment to the treatment discontinuation visit. Patients are permitted to use the following concomitant therapies during the study.

Systemic corticosteroids and TNF-α inhibitors may attenuate potential beneficial immunologic effects of treatment with atezolizumab. Therefore, in situations in which systemic corticosteroids or TNF-α inhibitors would be routinely administered, alternatives, including antihistamines, should be considered. If the alternatives are not feasible, systemic corticosteroids and TNF-α inhibitors may be administered at the discretion of the investigator.

Premedication with antihistamines, antipyretics, and/or analgesics may be administered for the second and subsequent study treatment infusions only, at the discretion of the investigator.

C. Efficacy Endpoints

To evaluate the efficacy of tiragolumab in combination with atezolizumab compared with atezolizumab monotherapy, the objective response rate (ORR), with ORR defined as the percentage of patients who have experienced a complete response (CR) or a partial response (PR) on two consecutive occasions greater than or equal to 4 weeks apart (as determined by the investigator according to RECIST v1.1), is measured as a primary endpoint. The primary efficacy analysis takes place once all patients have been enrolled and a minimum follow-up of approximately 6 months has been achieved among those patients, who remain in follow-up for ORR assessment. In the primary analysis, patients whose ORR assessment was missing are counted as not achieving a response. A one-sample z-test for proportion is used for comparing the ORR of the tiragolumab in combination with atezolizumab arm to the historical reference. An estimate of the ORR and its 95% CI (Clopper-Pearson; Clopper and Pearson 1934) is calculated for each treatment arm. It is expected that the lower end of the 95% CI of ORR in the combination arm (Table 61) and the observed ORR in the monotherapy arm (Table 62) should exclude a reference point.

TABLE 61
Overall Statistical Power in the Comparison of ORR in Tiragolumab in
Combination with Atezolizumab Arm to a Reference Point of 14.6%

| | True Underlying ORR in Tiragolumab | | | | |
|---|---|---|---|---|---|
| | in Combination with Atezolizumab | | | | |
| Reference ORR | 25% | 30% | 32.5% | 35% | 40% |
| 14.6% ^{a} (MDO ^{b} = 21%) | 85% | 98% | 100% | 100% | 100% |

MDO = minimum detectable observation;
ORR = overall response rate
^{a} Source: Chung et al. 2019.
^{b} MDO is defined as the smallest observed ORR leading to statistical significance at final analysis.

TABLE 62
Probability of Observed ORR in Atezolizumab Monotherapy
Arm Greater than a Reference Point at the Final Analysis for ORR

| | True Underlying ORR in | | | |
|---|---|---|---|---|
| | Atezolizumab Monotherapy | | | |
| Reference ORR | 5% | 10% | 15% | 20% |
| 14.6% ^{a} | 1% | 21% | 57% | 84% |

ORR = overall response rate.

The secondary efficacy objective for this study is to evaluate the efficacy of tiragolumab in combination with atezolizumab and of atezolizumab monotherapy on the basis of duration of response (DOR), disease control rate (DCR), best clinical response (BCR), Progression-free survival (PFS), and overall survival (OS). DOR is defined for patients who had an objective response as the time from the first occurrence of a documented objective response (CR or PR) to the date of disease progression or death from any cause (whichever occurs first), as determined by the IRC according to RECIST v1.1. DCR is defined as the proportion of patients with a CR, PR, or SD, as determined by the IRC according to RECIST v1.1. BCR is defined as the proportion of patients with a CR, PR, or SD, as determined by the investigator. PFS is defined as the time from randomization to the first occurrence of disease progression or death from any cause (whichever occurs first), as determined by the IRC according to RECIST v1.1. OS is defined as the time from randomization to death from any cause.

Additional exploratory efficacy endpoints may further include disease control (defined as SD for ≥6 weeks or a CR or PR), as determined by the investigator according to RECIST v1.1.

To evaluate the safety and tolerability of tiragolumab in combination atezolizumab compared with atezolizumab monotherapy, the incidence, nature, and severity of adverse events (AEs) (e.g., AEs graded according to the National Cancer Institute Common Terminology Criteria for Adverse Events version 5.0 (NCI CTCAE v5.0)) are measured (adverse events not specifically listed in NCI CTCAE are measured according to Table 63). Severity for cytokine-release syndrome (CRS) is also determined according to the American Society for Transplantation and Cellular Therapy CRS Consensus Grading scale. Additionally, clinically significant changes in vital signs, physical findings, and clinical laboratory results from baseline during and following administration of tiragolumab in combination with atezolizumab compared with atezolizumab monotherapy are also measured.

TABLE 63
Adverse Event Severity Grading Scale for Events Not Specifically Listed in NCI CTCAE

| Grade | Severity |
|---|---|
| 1 | Mild; asymptomatic or mild symptoms; clinical or |
| | diagnostic observations only; or intervention not indicated |
| 2 | Moderate; minimal, local, or non-invasive intervention indicated; |
| | or limiting age-appropriate instrumental activities of daily living^{a} |
| 3 | Severe or medically significant, but not immediately life-threatening; |
| | hospitalization or prolongation of hospitalization indicated; |
| | disabling; or limiting self-care activities of daily living^{b} |
| 4 | Life-threatening consequences or urgent intervention indicated |
| 5 | Death related to adverse event |

^{a}Instrumental activities of daily living refer to preparing meals, shopping for groceries or clothes, using the telephone, managing money, etc.
^{b}Examples of self-care activities of daily living include bathing, dressing and undressing, feeding oneself, using the toilet, and taking medications, as performed by patients who are not bedridden.

Timeframe of Outcome Measures

The timeframe of the primary outcome measure, objective response rate (ORR), will be the first occurrence of a documented objective response to the date of disease progression or death from any cause, whichever occurs first (up to 36 months). The timeframes of the secondary outcome measures can be found in Table 64.

TABLE 64
Timeframe of Secondary Outcome Measures

| Outcome Measure | Timeframe |
|---|---|
| Percentage of | Up to 36 months |
| Participants with | |
| Adverse Events | |
| Duration of | First occurrence of a documented objective response |
| Response (DOR) | to the date of disease progression or death from any |
| | cause, whichever occurs first (up to 36 months) |
| Disease Control | First occurrence of a documented control date to |
| Rate (DCR) | the date of disease progression or death from any |
| | cause, whichever occurs first (up to 36 months) |
| Best Clinical | First occurrence of a documented clinical response |
| Response (BCR) Rate | to the date of disease progression or death from any |
| | cause, whichever occurs first (up to 36 months) |
| Progression-Free | Time from randomization to the first occurrence |
| Survival (PFS) | of disease progression or death from any |
| | cause, whichever occurs first (up to 36 months) |
| PFS Rate at 6 Months | 6 months |
| Overall Survival (OS) | Time from randomization to death |
| | from any cause (up to 36 months) |
| OS Rate at 6 Months | 6 months, 12 months |
| and 12 Months | |
| Minimum Serum | Predose and postdose on Day 1 of Cycle 1 (each cycle is 21 days) |
| Concentration (Cmin) | and predose on Day 1 of Cycles 2, 3, 4, 8, 12 and 16 and at |
| of Tiragolumab | treatment discontinuation (TD) visit (up to 36 months) |
| Maximum Serum | Predose and postdose on Day 1 of Cycle 1 (each cycle is 21 days) |
| Concentration (Cmax) | and predose on Day 1 of Cycles 2, 3, 4, 8, 12 |
| of Tiragolumab | and 16 and at TD visit (up to 36 months) |
| Cmin of | Predose and postdose on Day 1 of Cycle 1 (each cycle is 21 days) |
| Atezolizumab | and predose on Day 1 of Cycles 2, 3, 4, 8, 12 |
| | and 16 and at TD visit (up to 36 months) |
| Cmax of | Predose and postdose on Day 1 of Cycle 1 (each cycle is 21 days) |
| Atezolizumab | and predose on Day 1 of Cycles 2, 3, 4, 8, 12 |
| | and 16 and at TD visit (up to 36 months) |
| Percentage | Predose on Day 1 of Cycles (each cycle is 21 days) |
| of Participants | 1, 2, 3, 4, 8, 12 and 16 and |
| with Anti-Drug | at TD visit (up to 36 months) |
| Antibodies | |
| (ADAs) to | |
| Tiragolumab | |
| Percentage | Predose on Day 1 of Cycles (each cycle is 21 days) |
| of Participants | 1, 2, 3, 4, 8, 12 and 16 and |
| with ADAs to | at TD visit (up to 36 months) |
| Atezolizumab | |

D. Biomarkers

Patient samples, including archival tumor tissues, as well as serum, plasma, whole blood, and stool are collected for exploratory biomarker assessments for all patients in the randomized study. In addition to assessing PD-L1 status, biomarkers related to resistance, disease progression, and clinical benefit of tiragolumab and/or atezolizumab are analyzed. For example, potential predictive and prognostic biomarkers related to the clinical benefit and safety of tiragolumab and/or atezolizumab are analyzed.

Tumor tissue and blood samples collected at baseline (and, if deemed clinically feasible by the investigator, tumor tissue collected at the time of disease progression) enables whole-exome sequencing (WES) and/or next-generation sequencing (NGS) to identify somatic mutations that are predictive of response to study treatment, are associated with progression to a more severe disease state, are associated with acquired resistance to study treatment, are associated with susceptibility to developing adverse events, or can increase the knowledge and understanding of disease biology.

Biomarkers include, but are not limited to, PD-L1 and/or TIGIT expression on tumor tissues, germline and somatic mutations from tumor tissue and/or from circulating tumor DNA in blood (including, but not limited to, mutation load, MSI, and MMR defects), identified through WGS and/or NGS, analysis of genes (e.g., CD274) or gene signatures associated with tumor immunobiology e.g., T_{effector} genes, PD-L1, TIGIT, HPV alterations, lymphocyte subpopulations, T cell-receptor repertoire, cytokines associated with T-cell and NK-cell activation, and plasma derived cytokines.

To assess the effect of the PD-L1/PD-1 pathway on ORR, PFS, DOR, and/or OS in the primary patient population, the relationship between protein, RNA, DNA, tumor mutational burden, and other exploratory biomarkers in tumor tissue and/or blood to efficacy, safety, PK, immunogenicity, and patient-reported outcomes (PROs) may be evaluated. Additionally, to assess the effect of the TIGIT pathway on ORR, PFS, DOR, and/or OS following in the primary population, ORR, DOR, PFS, and OS may be evaluated in a patient population whose tumors have TIGIT expression, as defined by protein and/or RNA expression.

Exploratory biomarker analyses may be performed in an effort to understand the association of these markers (e.g., TIGIT IHC status) with study treatment efficacy. The efficacy outcomes may be explored in a population of patients whose tumors have high TIGIT expression, as determined by IHC and/or RNA analysis. Exploratory analysis of WGS data may be conducted in the context of this study and explored in aggregate with data from other studies to increase researcher's understanding of disease pathobiology and guide the development of new therapeutic approaches.

E. Immunogenicity Analyses

To evaluate the immune response to tiragolumab and atezolizumab, the incidence of treatment-emergent anti-drug antibodies (ADAs) and their potential impact on safety, efficacy, and pharmacokinetics (PK) are assessed.

As of December 2019 in the Phase Ia portion of Study GO30103, no treatment emergent anti-drug antibodies (ADAs) against tiragolumab were detected. In the Phase Ib portion of Study GO301 03, 3 of 154 (1.9%) evaluable patients were positive for treatment emergent ADAs against tiragolumab. As of October 2019 in Study GO40290, 1 of 66 (1.5%) evaluated patients were positive for treatment emergent ADAs against tiragolumab. As of October 2019 in Study GO40290, 1 of 66 (1.5%) evaluated patients were positive for treatment emergent ADAs against tiragolumab, and the patient was only positive at one timepoint.

F. Pharmacokinetic Analyses

To characterize the pharmacokinetics of tiragolumab when given in combination with atezolizumab, serum concentrations of tiragolumab are determined from subjects at different time points. Further, to characterize the pharmacokinetics of atezolizumab when atezolizumab is administered in combination with tiragolumab or as a monotherapy, plasma concentration of atezolizumab is obtained from subjects at different time points during the study.

### Example 14. A Phase Ib, Open-Label, Multicohort Study of the Safety, Efficacy, and Pharmacokinetics of Tiragolumab in Combination with Atezolizumab and Chemotherapy in Patients with Triple-Negative Breast Cancer

The present example describes a Phase Ib, open-label, multicohort study designed to evaluate the safety, efficacy, and pharmacokinetics of tiragolumab in combination with atezolizumab and chemotherapy in patients with early triple-negative breast cancer (eTNBC). The study consists of the following cohort:
Cohort B enrolls patients (PD-L1 all-comer population) with clinically assessed T2-4d TNBC who are eligible for surgery. Patients are randomized in a 1:1 ratio to one of the following two treatment arms:
   Arm A: tiragolumab combined with atezolizumab, nab-paclitaxel, and carboplatin, followed by tiragolumab combined with atezolizumab, doxorubicin, and cyclophosphamide (referred to as tiragolumab and atezolizumab+nab-pac-carbo-AC)
   Arm B: tiragolumab in combination with atezolizumab, nab-paclitaxel, followed by tiragolumab combined with atezolizumab, doxorubicin, and cyclophosphamide (referred to as tiragolumab and atezolizumab+nab-pac-AC)

Additionally, AC and G-CSF (e.g., filgrastim or pegfilgrastim) or GM-CSF is administered as background treatment after nab-paclitaxel to patients in Cohort B.

The study is designed to enable the assessment of the safety and tolerability, preliminary efficacy, and pharmacokinetics of neoadjuvant tiragolumab combined with atezolizumab, nab-paclitaxel, and carboplatin followed by AC or neoadjuvant tiragolumab in combination with atezolizumab, nab-paclitaxel, followed by AC in patients who are eligible for surgery with initially clinically assessed T2-4d TNBC

A. Study Design

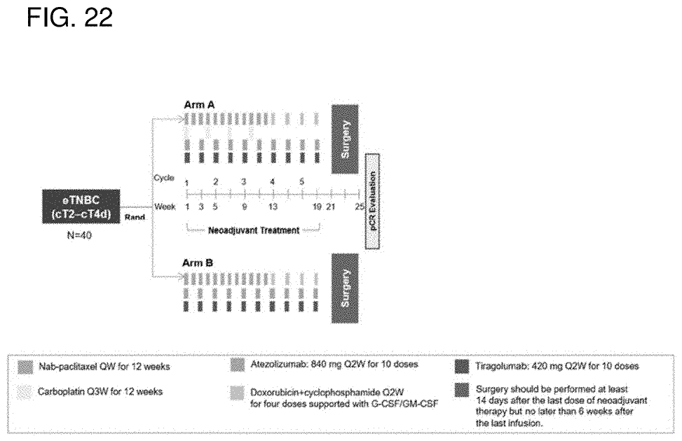

This Phase Ib, multicohort, open-label, multicenter, global study is designed to investigate the safety and tolerability, preliminary efficacy, and pharmacokinetics of neoadjuvant tiragolumab combined with atezolizumab, nab-paclitaxel, and carboplatin followed by doxorubicin and cyclophosphamide (AC) or neoadjuvant tiragolumab in combination with atezolizumab, nab-paclitaxel, followed by AC in patients who are eligible for surgery with initially clinically assessed T2-4d TNBC (PD-L1 all-comer population). FIG. 22 illustrates the study design for Cohort B.

Patients who have consented and are eligible are randomized in 1:1 ratio to one of the following two treatment arms:
Arm A (up to approximately 20 patients): Tiragolumab (420 mg) and atezolizumab (840 mg) are administered by IV infusion Q2W in combination with nab-paclitaxel (125 mg/m²) administered by IV infusion QW and carboplatin (area under the concentration-time curve [AUC]: 5 mg/mL/min]) administered by IV infusion Q3W for four cycles, followed by tiragolumab (420 mg) and atezolizumab (840 mg) in combination with doxorubicin (60 mg/m²) and cyclophosphamide (600 mg/m²) administered Q2W by IV infusion with granulocyte colony-stimulating factor (G-CSF; e.g., filgrastim or pegfilgrastim) or granulocyte macrophage colony-stimulating factor (GM-CSF) support for four doses.
Arm B (up to approximately 20 patients): Tiragolumab (420 mg) and atezolizumab (840 mg) are administered by IV infusion Q2W in combination with nab-paclitaxel (125 mg/m²) administered by IV infusion QW for 12 weeks, followed by tiragolumab (420 mg) and atezolizumab (840 mg) in combination with doxorubicin (60 mg/m²) and cyclophosphamide (600 mg/m²) administered Q2W by IV infusion with G-CSF (e.g., filgrastim or pegfilgrastim) or GM-CSF support for four doses.

The exploratory endpoint (pathological complete response (pCR); eradication of invasive tumor from both breast and lymph nodes (ypT0/is ypN0)) is established by local review following completion of neoadjuvant therapy and surgery. Surgery should be performed no earlier than 14 days after but no later than 6 weeks after the final dose of neoadjuvant therapy. Platelet counts should be checked prior to surgery and should be ≥75,000 cells/μL.

Postoperative patient management may include radiotherapy as clinically indicated, and management of patients who do not achieve a pCR should follow current standard-of-care guidelines.

It is recommended that patients with clinically positive axillary nodes assessed on physical examination or by any radiographic imaging at baseline undergo fine-needle aspiration or a core-needle biopsy prior to randomization and that an axillary lymph node dissection (ALND) or at least a sentinel lymph node biopsy (SLNB) is performed at the time of definitive surgery. The results of the baseline fine-needle aspiration or core-needle biopsy is used to determine nodal staging (according to the Anatomic Stage Groups of the Union for International Cancer Control/American Joint Committee on Cancer [UICC/AJCC], 8th edition), such that patients with a positive biopsy result should be staged as lymph node positive (N1-N3c), whereas patients with a negative or equivocal biopsy result should be staged as lymph node negative (NO) regardless of any other clinical measurements.

For patients with clinically or fine-needle biopsy or core-needle biopsy-proven negative axillary nodes at baseline, axillary surgical management after completion of neoadjuvant therapy may include an SLNB or ALND. If an SLNB is conducted, it is strongly recommended that more than one lymph node (two to three minimum) be removed and all patients with positive macrometastases in sentinel nodes should undergo an ALND regardless of the number of positive nodes.

Patients undergo both clinical and radiologic tumor assessment at scheduled intervals during the study.

In order to evaluate the mechanism of action of the drug combination in the tumor microenvironment and possible resistance mechanisms, tumor tissue may be collected predose on Day 1 of Cycle 2.

Tumor tissue is collected by biopsy, unless not clinically feasible, as assessed and documented by the investigator, and at the time of first evidence of disease progression (prior to the start of new anti-cancer treatment). The samples are used to enable analysis of tumor tissue biomarkers related to resistance or disease progression and clinical benefit of tiragolumab in combination with atezolizumab.

B. Dosing and Administration

The treatment regimens are summarized in FIG. 22.

On days of scheduled infusions of atezolizumab, tiragolumab, and chemotherapy, chemotherapy is to be administered after infusion of atezolizumab and tiragolumab.

After the atezolizumab infusion (840 mg), patients receive 420 mg tiragolumab.

Administration of atezolizumab, tiragolumab, and chemotherapy is performed in a monitored setting where there is immediate access to trained personnel and adequate equipment and medicine to manage potentially serious reactions.

Refer to the pharmacy manual for detailed instructions on drug preparation, storage, and administration.

Tiragolumab and Atezolizumab

Patients in Cohort B receive atezolizumab at a fixed dose of 840 mg administered by IV infusion Q2W on Days 1 and 15 of each 28-day cycle, followed by tiragolumab at a fixed dose of 420 mg administered by IV infusion Q2W also Days 1 and 15 of each 28-day cycle (see FIG. 22). The tiragolumab and atezolizumab dose are fixed and are not dependent on body weight. Tiragolumab and atezolizumab infusions (including observation periods) are administered according to the instructions outlined in Table 65.

TABLE 65
Administration of First and Subsequent Tiragolumab
Infusions and Atezolizumab Infusions

| | First Infusion | Subsequent Infusions |
|---|---|---|
| Atezolizumab | No premedication is permitted | If the patient experienced an infusion- |
| infusion | prior to the atezolizumab infusion, | related reaction with any previous |
| | Vital signs (pulse rate, respiratory rate, | infusion, premedication with an |
| | blood pressure, and temperature) are | antihistamine and/or antipyretic |
| | recorded within 60 minutes | medication may be administered |
| | prior to the infusion. | for subsequent doses at the |
| | Atezolizumab is infused | discretion of the investigator. |
| | over 60 (±15) minutes. | Vital signs are recorded within 60 |
| | If clinically indicated, vital signs | minutes prior to the infusion. |
| | should be recorded every 15 (±5) | Atezolizumab is infused over 30 (±10) |
| | minutes during the infusion and at 30 | minutes if the previous infusion was |
| | (±10) minutes after the infusion, | tolerated without an infusion-related |
| | Patients are informed about the | reaction, or 60 (±15) minutes if the |
| | possibility of delayed post-infusion | patient experienced an infusion-related |
| | symptoms and are instructed to | reaction with the previous infusion. |
| | contact their study physician if | If the patient experienced an infusion- |
| | they develop such symptoms. | related reaction with the previous infusion |
| | | or if clinically indicated, vital signs should |
| | | be measured during the infusion and 30 |
| | | (±10) minutes after the infusion. |
| Observation | After the infusion of atezolizumab, the | If the patient tolerated the previous |
| period after | patient begins a 60-minute observation | atezolizumab infusion well without |
| infusion of | period before the tiragolumab infusion. | infusion-associated adverse events, the |
| atezolizumab | Vital signs are recorded at | observation period before the tiragolumab |
| | 30 (±10) minutes after the | infusion may be reduced to 30 minutes. |
| | infusion of atezolizumab. | If the patient experienced infusion- |
| | | associated adverse events in the previous |
| | | infusion, the observation period |
| | | should be 60 minutes. |
| | | If clinically indicated, vital signs should |
| | | be recorded at 15 (±10) minutes after the |
| | | infusion of atezolizumab. |
| Infusion of | No premedication is permitted | If the patient experienced an infusion- |
| tiragolumab | prior to the tiragolumab infusion. | related reaction during any previous |
| | Vital signs (pulse rate, respiratory rate, | infusion of tiragolumab, premedication |
| | blood pressure, and temperature | with an antihistamine and/or antipyretic |
| | are recorded within 60 | medication may be administered |
| | minutes prior to the infusion. | for subsequent doses, at the |
| | Tiragolumab is infused | discretion of the investigator. |
| | over 60 (±10) minutes. | Vital signs should be recorded within 60 |
| | Vital signs are recorded every | minutes prior to the tiragolumab infusion. |
| | 15 (±5) minutes during | Tiragolumab should be infused over 30 |
| | the infusion and at 30 (±10) | (±10) minutes if the previous infusion was |
| | minutes after the infusion. | tolerated without an infusion-related |
| | | reaction, or 60 (±10) minutes if the |
| | | patient experienced an infusion-related |
| | | reaction with the previous infusion. |
| | | Vital signs should be recorded during |
| | | the infusion if clinically indicated. |
| Observation | After the infusion of tiragolumab, | If the patient tolerated the previous |
| period after | the patient begins a 60-minute | infusion of tiragolumab well without |
| infusion of | observation period before | infusion-associated adverse events, the |
| tiragolumab | subsequent chemotherapy infusions. | observation period before the subsequent |
| | Vital signs should be recorded | chemotherapy infusions may be |
| | at 30 (±10) minutes after the | reduced to 30 minutes. |
| | infusion of tiragolumab. | If clinically indicated, vital signs should |
| | Patients are informed about the | be recorded at 15 (±10) minutes |
| | possibility of delayed postinfusion | after the infusion of tiragolumab. |
| | symptoms and are instructed to contact | Patients are informed about the |
| | their study physician if they | possibility of delayed postinfusion |
| | develop such symptoms. | symptoms and are instructed to |
| | | contact their study physician if |
| | | they develop such symptoms. |

Nab-Paclitaxel

Nab-paclitaxel is administered to patients as an IV infusion given over 30 minutes.

Nab-paclitaxel should be administered after atezolizumab and tiragolumab. The dose of nab-paclitaxel is 125 mg/m² administered to patients by IV infusion QW for 12 weeks. Doses of nab-paclitaxel should not be administered more frequently than every 7 days.

Sites should follow their institutional standard of care for determining dose adjustments for nab-paclitaxel in the event of patient weight changes. The infusion site should be closely monitored for possible infiltration during study drug administration.

Refer to the local prescribing information for more details regarding the preparation and administration of nab-paclitaxel.

Carboplatin

Carboplatin is administered after the completion of nab-paclitaxel administration by short-term IV infusion over 15-60 minutes to target an AUC of 5 mg/mL/min every 3 weeks for 4 doses.

There is no known antidote for carboplatin overdose. If necessary, the patient may need supportive treatment relating to myelosupression and impairment of renal, hepatic, and auditory function. Doses of up to 1600 mg/m2 have been associated with patients feeling extremely ill with diarrhea and alopecia developing. Use of higher-than-recommended doses of carboplatin has also been associated with loss of vision. For further details, refer to the local prescribing information for carboplatin.

Cyclophosphamide

Cyclophosphamide should be given as an IV bolus over 3-5 minutes or as an IV infusion in accordance with the local standard of care. AC should be administered after atezolizumab and tiragolumab. The dose of cyclophosphamide is 600 mg/m² administered intravenously. Dose delays and dose reductions for toxicity are permitted. Cyclophosphamide is administered Q2W for four doses (dose-dense AC) with G-CSF or GM-CSF support. Note: Oral cyclophosphamide is not permitted.

Chemotherapy-induced nausea and vomiting prophylaxis and treatment should be administered as clinically indicated. Because systemic corticosteroids may attenuate the potential beneficial immunologic effects of treatment with atezolizumab and tiragolumab, alternative agents should be considered when clinically feasible.

Refer to the local prescribing information for details regarding the preparation and administration of cyclophosphamide.

Doxorubicin

Doxorubicin should be given as an IV bolus over 3-5 minutes or as an IV infusion given over 15-30 minutes, in accordance with local standards of care. AC should be administered after atezolizumab and tiragolumab. The dose of doxorubicin is 60 mg/m² administered to patients by IV infusion. Dose delays and reduction for toxicity are permitted. Doxorubicin is administered Q2W for four doses (dose-dense AC) with G-CSF or GM-CSF support.

Refer to the local prescribing information for details regarding the preparation and administration of doxorubicin

Pre-Medications and Supportive Care

In general, chemotherapy supportive care should be administered according to the American Society of Clinical Oncology (ASCO), European Organisation for Research and Treatment of Cancer (EORTC), or ESMO guidelines, or local standard of care.

Chemotherapy-induced nausea and vomiting prophylaxis and treatment should be administered as clinically indicated. Because systemic corticosteroids may attenuate the potential beneficial immunologic effects of treatment with atezolizumab and tiragolumab, alternative agents should be considered when clinically feasible, with the exception of the guidance provided in the protocol.

Prophylactic G-CSF or GM-CSF may be used to mitigate the risk of hematologic toxicities according to local policies and is required during the AC portion of chemotherapy. Treatment of neutropenia with G-CSF or GM-CSF is permitted according to local policies.

Dose Modifications

There are no dose modifications for atezolizumab or tiragolumab in this study. For management of nab-paclitaxel (i.e., dose modification and treatment interruption rules), refer to Table 25. For guidelines regarding the management (i.e., dose modification and treatment interruption rules) of carboplatin, doxorubicin, or cyclophosphamide-associated toxicities, please refer to the respective local prescribing information for each agent.

C. Concomitant Therapy

Concomitant therapy consists of any medication (e.g., prescription drugs, over-the-counter drugs, vaccines, herbal or homeopathic remedies, nutritional supplements) used by a patient in addition to protocol-mandated treatment from 7 days prior to initiation of study drug to the treatment discontinuation visit.

Permitted Therapy

Patients are permitted to use the following therapies during the study:
Oral contraceptives with a failure rate of <1% per year
Hormone-replacement therapy
Prophylactic or therapeutic anticoagulation therapy (such as warfarin at a stable dose or low-molecular-weight heparin)
Inactivated influenza vaccinations
Megestrol acetate administered as an appetite stimulant
Mineralocorticoids (e.g., fludrocortisone)
Corticosteroids administered for chronic obstructive pulmonary disease or asthma
Low-dose corticosteroids administered for orthostatic hypotension or adrenocortical insufficiency
Bisphosphonates for the prevention of skeletal events
Palliative radiotherapy (e.g., treatment of known bony metastases or symptomatic relief of pain) as outlined below:
   Palliative radiotherapy is permitted, provided it does not interfere with the assessment of tumor target lesions (e.g., the lesion to be irradiated must not be the only site of measurable disease). Treatment with tiragolumab and atezolizumab may be continued during palliative radiotherapy. Treatment with nab-paclitaxel should be interrupted per institutional standard of care.
G-CSF (e.g., filgrastim or pegfilgrastim) or GM-CSF treatment permitted for patients receiving chemotherapy (nab-paclitaxel) and required during the AC portion of chemotherapy
   Primary prophylaxis should be administered to patients according to the ASCO, EORTC, or ESMO guidelines or per local standard practice; namely, in patients who are ≥60 years old and/or with comorbidities (Smith et al. 2006; Crawford et al. 2010; Aapro et al. 2011).
   Evidence supporting the use of long-acting (pegylated) forms of G-CSF in patients receiving weekly nab-paclitaxel is limited and investigators should consider giving preference to conventional formulations of G-CSF.

Premedication with antihistamines, antipyretic medications, and/or analgesics may be administered for the second and subsequent atezolizumab and tiragolumab infusions only, at the discretion of the investigator.

Cautionary Therapy

Systemic corticosteroids and TNF-α inhibitors may attenuate potential beneficial immunologic effects of treatment with atezolizumab. Therefore, in situations in which systemic corticosteroids or TNF-α inhibitors would be routinely administered, alternatives, including antihistamines, should be considered. If the alternatives are not feasible, systemic corticosteroids and TNF-α inhibitors may be administered at the discretion of the investigator.

Systemic corticosteroids are recommended, at the discretion of the investigator, for the treatment of specific adverse events when associated with atezolizumab therapy.

Prohibited Therapy

Use of the following concomitant therapies is prohibited as described below:
Concomitant therapy intended for the treatment of cancer (including, but not limited to, chemotherapy, hormonal therapy, immunotherapy, radiotherapy, and herbal therapy), whether health authority-approved or experimental, for various time periods prior to starting study treatment, depending on the agent, and during study treatment, until disease progression is documented and the patient has discontinued study treatment with the exception of palliative radiotherapy
Investigational therapy within 28 days prior to initiation of study treatment and during study treatment
Live, attenuated vaccines (e.g., FLUMIST®) within 4 weeks prior to initiation of study treatment, during atezolizumab treatment, and for 5 months after the final dose of study treatment
Systemic immunostimulatory agents (including, but not limited to, interferons and IL-2) within 4 weeks or 5 drug-elimination half-lives (whichever is longer) prior to initiation of study treatment and during study treatment because these agents could potentially increase the risk for autoimmune conditions when given in combination with atezolizumab and tiragolumab
Systemic immunosuppressive medications (including, but not limited to, azathioprine, methotrexate, and thalidomide) during study treatment because these agents could potentially alter the efficacy and safety of atezolizumab and tiragolumab

Additional Restrictions Related to Carboplatin Chemotherapy
Concomitant use of yellow fever vaccine is prohibited, and vaccination with other live attenuated vaccine should be avoided.
Killed or inactivated vaccines may be administered; however, the response to such vaccines may be diminished.
Concomitant use of phenytoin or fosphenytoin is not recommended owing to the risk of exacerbation of convulsions, risk of toxicity enhancement, or loss of efficacy of the cytotoxic drug.
The concomitant use of the following agents should be approached with caution: cyclosporine, nephrotoxic, or ototoxic drugs (e.g., aminoglycosides, vancomycin, capreomycin and diuretics, including loop diuretic agents [risk of increased, exacerbated, or cumulative toxicity]).
Concurrent (given together or ≤7 days apart) radiotherapy should be avoided.

D. Inclusion Criteria

Patients must meet the following general criteria for study entry:
Signed Informed Consent Form
Women and men age ≥18 years at time of signing Informed Consent Form
Ability to comply with the study protocol
ECOG Performance Status of 0 or 1 (see Appendix 5)
Adequate hematologic and end-organ function, defined as the following laboratory results obtained within 14 days prior to initiation of study treatment (Day 1 of Cycle 1):
ANC ≥1.5×109/L (1500/μL) without G-CSF support within 2 weeks prior to Day 1 of Cycle 1
Lymphocyte count ≥0.5×109/L (≥500/μL)
Platelet count ≥100×109/L (≥100,000/μL) without transfusion within 2 weeks prior to Day 1 of Cycle 1
Hemoglobin 90 g/L (9 g/dL)
   Patients may be transfused or receive erythropoietic treatment to meet this criterion.
AST, ALT, and ALP ≤2.5×upper limit of normal (ULN)
Serum bilirubin ≤1.5×ULN with the following exception:
   Patients with known Gilbert disease who have serum bilirubin level ≤3×ULN may be enrolled.
INR and aPTT≤1.5×ULN
   This applies only to patients who are not receiving therapeutic anticoagulation; patients receiving therapeutic anticoagulation should be on a stable dose.
Creatinine clearance ≥30 mL/min (calculated using the Cockcroft-Gault formula)
Serum albumin ≥25 g/L (≥2.5 g/dL)
Negative HIV test at screening
Negative hepatitis B surface antigen (HBsAg) test at screening
Positive hepatitis B surface antibody (HBsAb) test at screening, or negative HBsAb at screening accompanied by either of the following:
Negative total hepatitis B core antibody (HBcAb)
Positive total HBcAb test followed by quantitative hepatitis B virus (HBV) DNA <500 IU/mL
   The HBV DNA test is performed only for patients who have a negative HBsAg test, a negative HBsAb test, and a positive total HBcAb test.
Negative hepatitis C virus (HCV) antibody test at screening, or positive HCV antibody test followed by a negative HCV RNA test at screening
   The HCV RNA test is performed only for patients who have a positive HCV antibody test.
For women of childbearing potential: agreement to remain abstinent (refrain from heterosexual intercourse) or use contraceptive methods, and agreement to refrain from donating eggs, as defined below:
   Women must remain abstinent or use contraceptive methods that result in a failure rate of <1% per year during the treatment period and for at least 5 months after the final dose of atezolizumab, 90 days after the final dose of tiragolumab, 1 month after the final dose of nab-paclitaxel, 6 months after the final dose of carboplatin or doxorubicin, or 12 months after the final dose of cyclophosphamide, whichever is later. Women must refrain from donating eggs during this same period.
   A woman is considered to be of childbearing potential if she is postmenarcheal, has not reached a postmenopausal state (≥12 continuous months of amenorrhea with no identified cause other than menopause), and has not undergone surgical sterilization (removal of ovaries and/or uterus). Examples of contraceptive methods with a failure rate of <1% per year, when used consistently and correctly, include combined (estrogen and progestogen containing) hormonal contraception associated with inhibition of ovulation, progestogen-only hormonal contraception associated with inhibition of ovulation, bilateral tubal occlusion; male sterilization; intrauterine devices; intrauterine hormone-releasing system; and sexual abstinence.
   The reliability of sexual abstinence should be evaluated in relation to the duration of the clinical study and the preferred and usual lifestyle of the patient.
   Periodic abstinence (e.g., calendar, ovulation, symptothermal, or postovulation methods) and withdrawal are not acceptable methods of contraception.
   Women who would like to become pregnant after discontinuation of study treatment should seek advice about oocyte preservation prior to initiation of study treatment because of the possibility of irreversible infertility due to treatment with chemotherapy.
For men: agreement to remain abstinent (refrain from heterosexual intercourse) or use contraceptive measures and agreement to refrain from donating sperm, as defined below:
   With female partners of childbearing potential or pregnant female partners, men must remain abstinent or use a condom plus an additional contraceptive method that together result in a failure rate of <1% per year during the treatment period and for 90 days after the final dose of tiragolumab, or 6 months after the final dose of nab-paclitaxel, carboplatin, doxorubicin, or cyclophosphamide, whichever is later. Men must refrain from donating sperm during this same period.
   The reliability of sexual abstinence should be evaluated in relation to the duration of the clinical trial and the preferred and usual lifestyle of the patient.
   Periodic abstinence (e.g., calendar, ovulation, symptothermal, or postovulation methods) and withdrawal are not acceptable methods of contraception.
   Men who would like to father a child after initiation of study treatment should seek advice about sperm preservation prior to initiation of study treatment because of the possibility of irreversible infertility due to treatment with chemotherapy.
For women who are not postmenopausal (≥12 months of non-therapy-induced amenorrhea) or have undergone a sterilization procedure, a negative serum pregnancy test result is required within 14 days prior to initiation of study treatment
Willingness and ability to comply with scheduled visits, treatment plans, laboratory tests, and other study procedures

Patients must meet the following cancer-specific eligibility criteria for entry:
Histologically documented TNBC (negative HER2, ER, and PR status, as assessed by local testing) HER2 negativity is defined as either of the following (Wolff et al. 2018) by local laboratory assessment: ISH non-amplified (HER2 to CEP17 ratio <2.0 or single probe average HER2 gene copy number <4 signals/cell or IHC 0 or IHC 1+ER and PgR negativity are defined as <1% of cells expressing hormonal receptors on IHC analysis.
   Patients with multifocal tumors (more than one discrete tumor confined to the same quadrant as the primary tumor) are eligible, provided at least one focus is sampled, locally confirmed, and documented as TNBC.
   Patients with multicentric tumors (multiple tumors involving more than one discrete quadrant) are eligible provided all discrete lesions are sampled, locally confirmed, and documented as TNBC.
Confirmed tumor PD-L1 evaluation as documented through central testing of a representative FFPE tumor tissue specimen in paraffin blocks (preferred) or at least 20 unstained slides
   Tumor tissue should be of good quality based on total and viable tumor content and must be evaluated for PD-L1 expression, as determined using the Ventana (SP142) PD-L1 IHC assay prior to enrollment, with positivity defined as 1% of the tumor area occupied by PD-L1-expressing tumor-infiltrating immune cells of any intensity as determined by the central laboratory. Patients whose tumor tissue is not evaluable for PD-L1 expression are not eligible.
   If multiple tumor specimens are submitted, patients may be eligible if at least one specimen is evaluable for PD-L1. Acceptable samples include core-needle biopsies for deep tumor tissue (a minimum of three cores) or excisional, incisional, punch, or forceps biopsies for cutaneous, subcutaneous, or mucosal lesions.
   FFPE tumor specimens in paraffin blocks are preferred.
   Fine-needle aspiration, brushing, cell pellet from pleural effusion, bone metastases, and lavage samples are not acceptable.
Primary breast tumor size of >2 cm on at least one radiographic or clinical measurement
Stage at presentation: cT2-cT4, cN0-cN3, and cM0
Patient agreement to undergo appropriate surgical management (e.g., including axillary lymph node surgery and partial or total mastectomy) after completion of neoadjuvant treatment
Baseline left ventricular ejection fraction (LVEF) ≥53%, as measured on echocardiogram (ECHO) or multiple-gated acquisition (MUGA) scan

E. Exclusion Criteria

Patients who meet any of the following criteria are excluded from study:
Pregnancy or breastfeeding, or intention of becoming pregnant during study treatment, within 90 days after tiragolumab treatment, within 5 months after atezolizumab treatment, within 6 months after nab-paclitaxel, carboplatin, or doxorubicin treatment, or within 12 months after cyclophosphamide treatment, whichever is longer
   Women of childbearing potential must have a negative serum pregnancy test result within 14 days prior to initiation of study treatment.
Evidence of significant uncontrolled concurrent disease that could affect compliance with the protocol or interpretation of results, including significant liver disease (such as cirrhosis, uncontrolled major seizure disorder, or superior vena cava syndrome)
Significant cardiovascular disease, such as New York Heart Association cardiac disease (Class II or greater), myocardial infarction within 3 months prior to initiation of study treatment, unstable arrhythmias, or unstable angina
   Patients with a known LVEF <53% are excluded.
   Patients with known coronary artery disease or congestive heart failure who do not meet the criteria above.
Severe infection within 4 weeks prior to initiation of study treatment, including, but not limited to, hospitalization for complications of infection, bacteremia, or severe pneumonia
Treatment with oral or IV antibiotics within 2 weeks prior to initiation of study treatment Patients receiving routine antibiotic prophylaxis (e.g., to prevent chronic obstructive pulmonary disease exacerbation or for dental extraction) are eligible.
Major surgical procedure within 28 days prior to initiation of study treatment or anticipation of the need for a major surgical procedure during the study other than for diagnosis
   Placement of central venous access catheter(s) (e.g., port or similar) is not considered a major surgical procedure and is therefore permitted.
History of severe allergic, anaphylactic, or other hypersensitivity reactions to chimeric or humanized antibodies or fusion proteins
Known hypersensitivity or allergy to biopharmaceuticals produced in CHO cells or any component of the tiragolumab or atezolizumab formulation
Active or history of autoimmune disease, including, but not limited to, myasthenia gravis, myositis, autoimmune hepatitis, systemic lupus erythematosus, rheumatoid arthritis, inflammatory bowel disease, vascular thrombosis associated with antiphospholipid syndrome, Wegener's granulomatosis, Sjôgren syndrome, Guillain-Barré syndrome, multiple sclerosis, vasculitis, or glomerulonephritis
   Patients with a history of autoimmune-mediated hypothyroidism on a stable dose of thyroid replacement hormone are eligible for this study.
Patients with controlled Type 1 diabetes mellitus on a stable insulin dosing regimen are eligible for this study.
Patients with eczema, psoriasis, lichen simplex chronicus or vitiligo with dermatologic manifestations only (e.g., no psoriatic arthritis) are permitted provided that they meet the following conditions:
   Rash must cover <10% of body surface area
   Disease is well controlled at baseline and requires only low-potency topical corticosteroids
   No occurrence of acute exacerbations of the underlying condition requiring psoralen plus ultraviolet A radiation, methotrexate, retinoids, biologic agents, oral calcineurin inhibitors, or high-potency or oral corticosteroids within the previous 12 months
Prior allogeneic stem cell or solid organ transplantation
History of idiopathic pulmonary fibrosis (including pneumonitis), drug-induced pneumonitis, organizing pneumonia (i.e., bronchiolitis obliterans, cryptogenic organizing pneumonia), or evidence of active pneumonitis on the chest computed tomography (CT) scan at screening
   History of radiation pneumonitis in the radiation field (fibrosis) is permitted.
Positive EBV viral capsid antigen (VCA) IgM test at screening
   An EBV polymerase chain reaction (PCR) test should be performed as clinically indicated to screen for
   active infection or suspected chronic active infection.

Patients with a positive EBV PCR test are excluded.
Active tuberculosis
Receipt of a live, attenuated vaccine within 4 weeks prior to initiation of study treatment or anticipation that such a live, attenuated vaccine is required during the study
   Patients must agree not to receive live, attenuated vaccine (e.g., FluMist®) within 28 days prior to initiation of study treatment, during treatment, or within 5 months following the final dose of study treatment.
Prior treatment with CD137 agonists or immune checkpoint blockade therapies, including anti-CTLA-4, with an exception of anti-PD-1, or anti-PD-L1 therapeutic antibodies
Treatment with systemic immunostimulatory agents (including, but not limited to, interferons or IL-2) within 4 weeks or 5 drug-elimination half-lives of the drug (whichever is longer) prior to initiation of study treatment
Treatment with systemic immunosuppressive medications (including, but not limited to, prednisone, dexamethasone, azathioprine, methotrexate, thalidomide, and anti-tumor necrosis factor [anti-TNF] agents) within 2 weeks prior to initiation of study treatment, or anticipated requirement for systemic immunosuppressive medications during the study
   Patients who have received acute, low-dose, systemic immunosuppressant medications (e.g., a one-time dose of dexamethasone for nausea) may be enrolled in the study.
   The use of inhaled corticosteroids for chronic obstructive pulmonary disease, mineralocorticoids (e.g., fludrocortisone) for patients with orthostatic hypotension, and low-dose supplemental corticosteroids for adrenocortical insufficiency is allowed.
History of invasive breast cancer
Stage IV (metastatic) breast cancer
Prior systemic therapy for treatment and prevention of breast cancer
Previous therapy with anthracyclines, platinum, or taxanes for any malignancy

Note: Patients with non-pleomorphic LCIS (either untreated or treated with surgery) are eligible.
Synchronous, bilateral invasive breast cancer
Previous incisional and/or excisional biopsy of primary tumor and/or axillary lymph nodes
Axillary lymph node dissection prior to initiation of neoadjuvant therapy
History of other malignancy within 5 years prior to screening, with the exception of those with a negligible risk of metastasis or death (e.g., 5-year OS of >90%), such as adequately treated carcinoma in situ of the cervix, non-melanoma skin carcinoma, localized prostate cancer, or Stage I uterine cancer
History of cerebrovascular accident within 12 months prior to initiation of study treatment
Cardiopulmonary dysfunction, as defined as any of the following prior to initiation of study treatment:
   History of NCI CTCAE v5.0 Grade ≥3 symptomatic congestive heart failure or New York Heart Association Class II or greater cardiac disease
   Angina pectoris requiring anti-anginal medication, serious cardiac arrhythmia not controlled by adequate medication, severe conduction abnormality, or clinically significant valvular disease
   High-risk uncontrolled arrhythmias (i.e., atrial tachycardia with a heart rate >100 beats/min at rest, significant ventricular arrhythmia [ventricular tachycardia], higher-grade atrioventricular [AV] block [second-degree AV block Type 2 [Mobitz 2], or third-degree AV block])
   Significant symptoms (Grade ≥2) relating to left ventricular dysfunction, cardiac arrhythmia, or cardiac ischemia
   Myocardial infarction within 12 months prior to initiation of study treatment
   Uncontrolled hypertension (systolic blood pressure >180 mmHg and/or diastolic blood pressure >100 mmHg)
   Evidence of transmural infarction on ECG
   Requirement for oxygen therapy
Known allergy or hypersensitivity to the components of the nab-paclitaxel, cyclophosphamide, doxorubicin, or carboplatin formulation
Known allergy or hypersensitivity to G-CSF or GM-CSF formulation

F. Analysis

Safety Analysis

Patients are randomized in a 1:1 ratio to one of two arms in Cohort B to assess the safety profile of atezolizumab+nab-pac-carbo+AC relative to tiragolumab and atezolizumab+nab-pac+AC. No formal statistical comparison is performed on the primary safety endpoint, and the safety analyses is descriptive. Therefore, no formal power calculations were performed. The sample size is considered sufficient to provide a descriptive safety analysis to assess tolerability of atezolizumab+nab-pac-carbo-AC relative to tiragolumab and atezolizumab+nab-pac-AC. The planned sample size allows observation of adverse events with a true incidence rate of ≥10% with acceptable probability in each treatment arm and across both arms combined (see Table 66).

TABLE 66
Chance of Observing Adverse Events Given the True Incidence

| | Actual Probability of | Chance of Observing |
|---|---|---|
| | Experiencing One or More | One or More Events |
| | Events per Patient | during the Study |
| Within tiragolumab and | 0.1 | 0.64151 |
| atezolizumab + nab-pac- | | |
| carbo-AC treatment arm | | |
| of Cohort B (n = 20) | | |
| Within tiragolumab and | 0.1 | 0.64151 |
| atezolizumab + nab-pac- | | |
| AC treatment arm of | | |
| Cohort B (n = 20) | | |
| Treatment arms combined | 0.1 | 0.8715 |
| in Cohort B (n = 40) | | |

Safety assessments include the incidence, nature, and severity of adverse events, protocol-mandated vital signs, laboratory abnormalities, and other protocol-specified tests that are deemed critical to the safety evaluation of the study. Adverse events are graded according to the NCI CTCAE v5.0.

Safety is assessed through summaries of adverse events, changes in laboratory test results, changes in vital signs, study treatment exposures, and immunogenicity as measured by ADAs and is presented by treatment arm.

Verbatim descriptions of adverse events is mapped to MedDRA terms.

Treatment-emergent events (defined as events occurring on or after the first dose of study treatment are summarized by MedDRA term, appropriate MedDRA levels, and NCI CTCAE v5.0 grade, regardless of relationship to study drug as assessed by the investigator. For each patient, if multiple incidences of the same adverse events occur, the maximum severity reported is used in the summaries.

The following treatment-emergent adverse events are summarized separately: adverse events leading to withdrawal of study drug, adverse events leading to dose reduction or interruption, Grade 3 adverse events, Grade 5 adverse events, serious adverse events, and adverse events of special interest.

All deaths and causes of death are summarized.

Relevant laboratory values are summarized by timepoint, with NCI CTCAE Grade 3 and Grade 4 values identified, where appropriate. Changes in NCI CTCAE grade are tabulated by treatment arm.

Analyses of Exposure, Adverse Events, Laboratory, and Vital Sign Data

Safety is assessed through summaries of exposure to study treatment, adverse events, changes in laboratory test results, and changes in vital signs and ECGs.

Study treatment exposure (such as treatment duration, total dose received, and number of cycles and dose modifications) is summarized with descriptive statistics.

All verbatim adverse event terms are mapped to MedDRA thesaurus terms, and adverse event severity is graded according to NCI CTCAE v5.0. All adverse events, serious adverse events, adverse events leading to death, adverse events of special interest, and adverse events leading to study treatment discontinuation that occur on or after the first dose of study treatment (i.e., treatment-emergent adverse events) are summarized by mapped term, appropriate thesaurus level, and severity grade. For events of varying severity, the highest grade is used in the summaries. Deaths and cause of death are summarized.

Relevant laboratory, vital sign (pulse rate, respiratory rate, blood pressure, pulse oximetry, and temperature), and ECG data are displayed by time, with grades identified where appropriate. Additionally, a shift table of selected laboratory tests is used to summarize the baseline and maximum postbaseline severity grade. Changes in vital signs and ECGs are summarized.

Efficacy Analysis

Efficacy analyses use an ITT approach, wherein any enrolled patient is included in the analysis regardless of whether the patient receives any assigned study drug, with patients in Cohort B grouped according to the treatment assigned at randomization in a 1:1 ratio. Analyses based on subsets of the ITT population might also be conducted. Hypothesis tests are two sided unless otherwise indicated.

The exploratory efficacy objective for Cohort B is to investigate the efficacy of tiragolumab and atezolizumab+nab-pac-carbo-AC relative to tiragolumab and atezolizumab+nab-pac-AC on the basis of the following endpoints:
pCR, defined as eradication of tumor from both breast and lymph nodes (ypT0/is ypN0)
   This efficacy endpoint is established following completion of neoadjuvant therapy and surgery.
   Patients whose pCR assessment is missing are counted as not achieving a pCR.
EFS, defined as the time from randomization until documented disease recurrence, progression, or death from any cause
   Patients without an event at the time of analysis are censored on the date when they are last known to be alive and event-free.
   Patients with no postbaseline information are censored at the date of randomization.
OS, defined as the time from randomization to the date of death from any cause
   Patients who are not reported as having died at the time of analysis are censored at the date when they were last known to be alive. Patients who do not have information after baseline are censored at the date of randomization.

Pharmacokinetic Analysis

Samples are collected for PK analyses and to compare exposure in this study with that attained in previous studies. Serum concentrations of tiragolumab and atezolizumab and plasma concentrations of nab-paclitaxel, carboplatin, doxorubicin, and cyclophosphamide are reported as individual values and summarized (mean, standard deviation, coefficient of variation, median, range, geometric mean, and geometric mean coefficient of variation) by treatment arm and cycle, when appropriate and as data allow.

Individual and median serum tiragolumab and atezolizumab concentrations are plotted by treatment arm and day. Tiragolumab and atezolizumab concentration data may be pooled with data from other studies using an established population-PK model to derive PK parameters such as clearance, volume of distribution, and AUC, as warranted by the data. Potential correlations of relevant PK parameters with dose, safety, efficacy, or biomarker outcomes may be explored.

Immunogenicity Analysis

The immunogenicity analysis includes patients with any ADA assessments, with patients grouped according to treatment received. The number and proportion of treatment-emergent ADA-positive patients and ADA-negative patients during both the treatment and follow-up periods are summarized by treatment arm.

The relationship between ADA status and safety, efficacy, and PK endpoints may be analyzed and reported by means of descriptive statistics.

Biomarker Analysis

Although no formal statistical analysis of exploratory biomarkers is performed, data may be analyzed in the context of this study and in aggregate with data from other studies.

### Example 15. A Phase II, Randomized, Double-Blind Study of Atezolizumab Plus Tiragolumab and Atezolizumab Plus Placebo as First-Line Treatment in Patients with Recurrent/Metastatic PD-11-Positive Squamous Cell Carcinoma of the Head and Neck

The present example describes a Phase II, randomized, double-blind, global study designed to evaluate the efficacy and safety of atezolizumab plus tiragolumab and atezolizumab plus placebo as first-line (1 L) treatment in recurrent/metastatic PD-L1-positive squamous cell carcinoma of the head and neck (SCCHN). The study consists of the following arms:

Arm A: patients receive atezolizumab at a fixed dose of 1200 mg administered by IV infusion every 3 weeks (Q3W) on Day 1 of each 21-day cycle, followed by tiragolumab at a fixed dose of 600 mg administered to patients by IV infusion Q3W on Day 1 of each 21-day cycle.

Arm B: patients receive atezolizumab at a fixed dose of 1200 mg administered by IV infusion Q3W on Day 1 of each 21-day cycle, followed by placebo administered by IV infusion Q3W on Day 1 of each 21-day cycle.

A. Study Design

This Phase II, randomized, double-blind, global study is designed to evaluate the efficacy and safety of atezolizumab plus tiragolumab and atezolizumab plus placebo as 1 L treatment in recurrent/metastatic PD-L1-positive SCCHN. Eligible patients include male and female patients age 18 years with Eastern Cooperative Oncology Group (ECOG) Performance Status of 0 or 1 who have recurrent disease that is not suitable for local therapy with curative intent and/or metastatic PD-L1-positive SCCHN (tumor-associated immune-cell (TIC) 5%), and have not received prior systemic therapy for recurrent/metastatic disease.

During screening, tumor specimens from each potentially eligible patient are prospectively tested for PD-L1 expression, as assessed by a central laboratory, using the investigational VENTANA PD-L1 (SP263) Companion Diagnostics (CDx) Assay. Only patients who are PD-L1 positive with a TIC ≥5% assessed centrally are eligible. PD-L1 expression is defined as PD-L1 low with TIC of 5%-19% and PD-L1 high with a TIC ≥20%.

Eligible patients are stratified by human papillomavirus (HPV) status for oropharynx cancer (HPV-positive oropharynx cancer: yes vs. no) and PD-L1 status (PD-L1: low vs. high). Enrollment into each PD-L1 subgroup (PD-L1 low [TIC 5%-19%] and PD-L1 high [TIC 20%]) is capped at approximately 55% of the total planned enrollment (i.e., approximately 66 patients).

Eligible patients are randomized in a 2:1 ratio to receive either atezolizumab plus tiragolumab (Arm A) or atezolizumab plus placebo (Arm B):

Arm A: patients receive atezolizumab at a fixed dose of 1200 mg administered by IV infusion every 3 weeks (Q3W) on Day 1 of each 21-day cycle, followed by tiragolumab at a fixed dose of 600 mg administered to patients by IV infusion Q3W on Day 1 of each 21-day cycle.

Arm B: patients receive atezolizumab at a fixed dose of 1200 mg administered by IV infusion Q3W on Day 1 of each 21-day cycle, followed by placebo administered by IV infusion Q3W on Day 1 of each 21-day cycle.

Patients undergo tumor assessments at baseline, every 6 weeks (±7 days) for the first 30 weeks following Day 1 of Cycle 1, and every 9 weeks (±7 days) after completion of the Week 30 tumor assessment. Tumor assessments continue per schedule regardless of treatment delays until radiographic disease progression per RECIST v1.1 or loss of clinical benefit for patients who continue study treatment after radiographic disease progression per RECIST v1.1, withdrawal of consent, death, or study termination by the Sponsor, whichever occurs first. At the investigator's discretion, scans must be performed at any time if progressive disease or loss of clinical benefit is suspected.

Patients who discontinue study treatment (for any reason, including, but not limited to, clinical decline or toxicity) in the absence of radiographic disease progression per RECIST v1.1 continue scheduled tumor assessments at the frequency described above until radiographic disease progression per RECIST v1.1, withdrawal of consent, death, or study termination by the Sponsor, whichever occurs first. In the absence of radiographic disease progression per RECIST v1.1, tumor assessments should continue regardless of whether a patient starts a new anti-cancer therapy. Objective response at a single timepoint is determined by the investigator according to RECIST v1.1.

Serum samples are collected to monitor atezolizumab and tiragolumab PK and to detect the presence of antibodies to atezolizumab and tiragolumab. Patient samples, including archival and fresh tumor tissue, plasma, and blood samples arecollected for exploratory biomarker assessments.

Safety assessments include the incidence, nature, and severity of adverse events, and other protocol-specified test, such as laboratory abnormalities, that are deemed critical to the safety evaluation of the study.

After study treatment discontinuation, survival follow-up information is collected by means of telephone calls, patient medical records, and/or clinic visits approximately every 3 months until death, loss to follow-up, or study termination by the Sponsor, whichever occurs first. All patients are to be periodically contacted for survival and new anti-cancer therapy information unless the patient requests to be withdrawn from survival follow-up (this request must be documented in the source documents and signed by the investigator). If the patient withdraws from survival follow-up, study staff may use a public information source (e.g., county records) to obtain information about survival status.

Treatment may be continued as long as patients are experiencing clinical benefit, as assessed by the investigator, in the absence of unacceptable toxicity or symptomatic deterioration attributed to disease progression after an integrated assessment or radiographic data, biopsy results (if available), and clinical status. Patients who meet the criteria for disease progression per RECIST v1.1, but continue to have clinical benefit, are permitted to continue treatment (atezolizumab plus tiragolumab or atezolizumab plus placebo) if they meet all of the outlined criteria and provide written consent.

B. Dosing and Administration

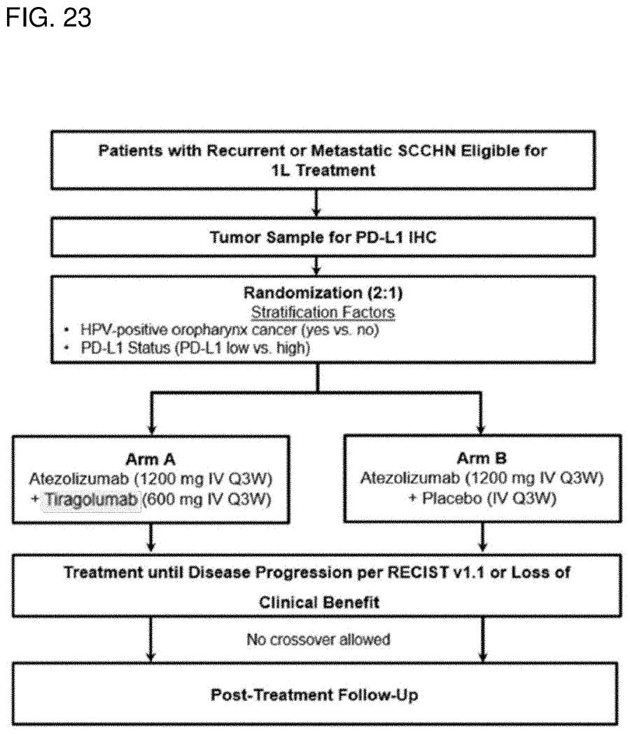

The treatment regimens are summarized in FIG. 23.

Atezolizumab

All patients receive 1200 mg atezolizumab administered by IV infusion on Day 1 of each 21-day cycle. The atezolizumab dose is fixed and is not dependent on body weight. Atezolizumab infusions are administered per the instructions outlined in Table 67.

TABLE 67
Administration of First and Subsequent Atezolizumab Infusions

| | Day 1, Cycle 1 Infusion | Day 1 Infusion of Subsequent Cycles |
|---|---|---|
| Atezolizumab | No premedication is permitted | If the patient experienced an infusion- |
| infusion | prior to the atezolizumab infusion. | related reaction with any previous infusion |
| | Vital signs (pulse rate, respiratory | of atezolizumab, premedication with an |
| | rate, blood pressure, and temper- | antihistamine and/or antipyretic |
| | ature) are recorded within 60 | medication may be administered for |
| | minutes prior to the infusion. | subsequent doses at the discretion of the |
| | Atezolizumab is infused over | investigator. |
| | 60 (±15) minutes. | Vital signs are recorded within 60 |
| | If clinically indicated, vital signs | minutes prior to the infusion. |
| | are recorded every 15 (±5) minutes | Atezolizumab is infused over 30 (±10) |
| | during the infusion. | minutes if the previous infusion was |
| | | tolerated without an infusion-related |
| | | reaction, or 60 (±15) minutes if the |
| | | patient experienced an infusion-related |
| | | reaction with the previous infusion. |
| | | If the patient experienced an infusion- |
| | | related reaction with the previous infusion |
| | | or if clinically indicated, vital signs are |
| | | recorded during the infusion. |
| Observation | After the infusion of atezolizumab, | If the patient tolerated the previous |
| period after | the patient begins a 60-minute | atezolizumab infusion well without |
| atezolizumab | observation period. | infusion-associated adverse events, the |
| infusion | Vital signs are recorded at 30 | observation period after the next and |
| | (±10) minutes after the infusion | following infusions may be reduced to 30 |
| | of atezolizumab. | minutes. |
| | Patients are informed about the | If the patient experienced infusion- |
| | possibility of delayed postinfusion | associated adverse events in the previous |
| | symptoms and instructed to | infusion, the observation period are 60 |
| | contact their study physician if | minutes. |
| | they develop such symptoms. | If clinically indicated, vital signs are |
| | | recorded at 30 (±10) minutes after the |
| | | infusion of atezolizumab. |

Tiragolumab and Placebo

Following the administration of atezolizumab and an observation period (see Table 67), patients receive 600 mg tiragolumab/placebo administered by IV infusion on Day 1 of each 21-day cycle. The tiragolumab/placebo dose is fixed and is not dependent on body weight. Tiragolumab/placebo infusions are administered per the instructions outlined in Table 68.

TABLE 68
Administration of First and Subsequent Infusions of Tiragolumab/Placebo

| | Day 1, Cycle 1 Infusion | Day 1 Infusion of Subsequent Cycles |
|---|---|---|
| Infusion of | No premedication is permitted prior | If the patient experienced an infusion- |
| tiragolumab/placebo | to the tiragolumab/placebo infusion. | related reaction with any previous |
| | Vital signs (pulse rate, respiratory | infusion of tiragolumab/placebo, |
| | rate, blood pressure, and | premedication with an antihistamine |
| | temperature) are recorded within 60 | and/or antipyretic medication may be |
| | minutes prior to the infusion. | administered for subsequent doses at |
| | Tiragolumab/placebo is infused | the discretion of the investigator. |
| | over 60 (±15) minutes. | Vital signs are recorded within 60 |
| | Vital signs are recorded every 15 | minutes prior to the |
| | (±5) minutes during the infusion. | tiragolumab/placebo infusion. |
| | | Tiragolumab/placebo is infused over |
| | | 30 (±10) minutes if the previous |
| | | infusion was tolerated without an |
| | | infusion-related reaction, or 60 (±15) |
| | | minutes if the patient experienced an |
| | | infusion-related reaction with the |
| | | previous infusion. |
| | | Viral signs are recorded during the |
| | | infusion if clinically indicated. |
| Observation period | After the infusion of | If the patient tolerated the previous |
| after infusion of | tiragolumab/placebo, the patient | infusion of tiragolumab/placebo well |
| tiragolumab/placebo | begins a 60-minute observation | without infusion-associated adverse |
| | period. | events, the observation period after the |
| | Vital signs are recorded at 30 (±10) | next and following infusions may be |
| | minutes after the infusion of | reduced to 30 minutes. |
| | tiragolumab/placebo. | If the patient experienced infusion- |
| | Patients are informed about the | associated adverse events in the |
| | possibility of delayed postinfusion | previous infusion, the observation |
| | symptoms and instructed to contact | period are 60 minutes. |
| | their study physician if they | If clinically indicated, vital signs are |
| | develop such symptoms. | recorded at 30 (±10) minutes after the |
| | | infusion of tiragolumab/placebo. |
| | | Patients are informed about the |
| | | possibility of delayed postinfusion |
| | | symptoms and instructed to contact |
| | | their study physician if they develop |
| | | such symptoms. |

Atezolizumab and Tiragolumab/Placebo

The following rules apply as long as neither atezolizumab nor tiragolumab/placebo has been permanently discontinued:
Treatment cycles normally begin with dosing of atezolizumab and tiragolumab/placebo on Day 1 of each 21-day cycle. If either study drug is delayed due to a drug-related toxicity, it is recommended that the other study drug is also delayed because the safety profiles for atezolizumab and tiragolumab are similar; however, a cycle may begin with the administration of the other study drug if considered appropriate at the discretion of the investigator.
In case of delays in dosing of one study drug for drug-related toxicity while the other study drug is given as planned, it is recommended that the study drug being delayed are administered at the next scheduled infusion (i.e., at the next scheduled 21-day cycle).

Dose Modifications

There are no dose modifications, including dose reductions, for atezolizumab or tiragolumab/placebo in this study.

C. Concomitant Therapy

Concomitant therapy consists of any medication (e.g., prescription drugs, over-the-counter drugs, vaccines, herbal or homeopathic remedies, nutritional supplements) used by a patient in addition to protocol-mandated treatment from 7 days prior to initiation of study treatment to the treatment discontinuation visit.

Permitted Therapy

Patients are permitted to use the following therapies during the study:
Oral contraceptives with a failure rate of <1% per year
Hormone-replacement therapy
Prophylactic or therapeutic anticoagulation therapy (such as warfarin at a stable dose or low-molecular-weight heparin)
Inactivated influenza vaccinations
Megestrol acetate administered as an appetite stimulant
Mineralocorticoids (e.g., fludrocortisone)
Inhaled or low-dose corticosteroids administered for COPD or asthma
Low-dose corticosteroids administered for orthostatic hypotension or adrenocortical insufficiency
Palliative radiotherapy (e.g., treatment of known bony metastases or symptomatic relief of pain) as outlined below:
   In patients without documentation of radiographic disease progression per RECIST v1.1, it is strongly encouraged to maximize supportive care for symptomatic management and to avoid radiotherapy that interfere with the assessment of tumor target lesions.
   Treatment with atezolizumab and tiragolumab/placebo may be continued during palliative radiotherapy.
   Premedication with antihistamines, antipyretics, and/or analgesics may be administered for the second and subsequent atezolizumab and tiragolumab/placebo infusions only, at the discretion of the investigator.
      In general, investigators should manage a patient's care (including preexisting conditions) with supportive therapies other than those defined as cautionary or prohibited therapies as clinically indicated, per local standard practice. Patients who experience infusion-associated symptoms may be treated symptomatically with acetaminophen, ibuprofen, diphenhydramine, and/or H2-receptor antagonists (e.g., famotidine, cimetidine), or equivalent medications per local standard practice. Serious infusion-associated events manifested by dyspnea, hypotension, wheezing, bronchospasm, tachycardia, reduced oxygen saturation, or respiratory distress should be managed with supportive therapies as clinically indicated (e.g., supplemental oxygen and β2-adrenergic agonists).

Cautionary Therapy

Systemic corticosteroids and TNF-α inhibitors may attenuate potential beneficial immunologic effects of treatment with atezolizumab and/or tiragolumab. Therefore, in situations in which systemic corticosteroids or TNF-α inhibitors are routinely administered, alternatives, including antihistamines, should be considered. If the alternatives are not feasible, systemic corticosteroids and TNF-α inhibitors may be administered at the discretion of the investigator.

Systemic corticosteroids are recommended, at the discretion of the investigator, for the treatment of specific adverse events when associated with atezolizumab and/or tiragolumab therapy.

Prohibited Therapy

Use of the following concomitant therapies is prohibited as described below:
Concomitant therapy intended for the treatment of cancer (including, but not limited to surgery, chemotherapy, hormonal therapy, immunotherapy, radiotherapy, and herbal therapy), whether health authority-approved or experimental, is prohibited for various time periods prior to starting study treatment, depending on the agent, and during study treatment, until disease progression is documented and the patient has discontinued study treatment, with the exception of palliative radiotherapy and local therapy under certain circumstances.
Investigational therapy is prohibited within 28 days prior to initiation of study treatment and during study treatment.
Live, attenuated vaccines (e.g., FLUMIST®) are prohibited within 4 weeks prior to initiation of study treatment, during study treatment, and for 5 months after the final dose of atezolizumab and 90 days after last dose of tiragolumab/placebo.
Systemic immunostimulatory agents (including, but not limited to, interferons and IL-2) are prohibited within 4 weeks or 5 drug-elimination half-lives (whichever is longer) prior to initiation of study treatment and during study treatment because these agents could potentially increase the risk for autoimmune conditions when given in combination with study treatment.
Systemic immunosuppressive medications (including, but not limited to, cyclophosphamide, azathioprine, methotrexate, and thalidomide) are prohibited during study treatment because these agents could potentially alter the efficacy and safety of study treatment.

D. Inclusion Criteria

Patients must meet the following criteria for study entry:
Signed Informed Consent Form
Age ≥18 years at time of signing Informed Consent Form
Ability to comply with the study protocol
Histologically or cytologically confirmed recurrent/metastatic SCCHN involving the oropharynx, oral cavity, larynx, or hypopharynx, that is considered incurable by local therapies
   Patients with a primary site of tumor in the nasopharynx or parotid gland, of any histology, are not eligible.
Known results from HPV status test for oropharyngeal carcinoma as determined locally by p16 immunohistochemistry (IHC), in situ hybridization, or by polymerase chain-based assay
   Note: HPV testing for oral cavity, larynx, and hypopharynx cancers is not required.
No prior systemic therapy for metastatic and/or recurrent SCCHN
   Patients who received systemic therapies given as part of multi-modal treatment (e.g., concurrent chemo-radiation) for locally advanced disease and whose last dose of systemic therapy was 6 months prior to randomization are permitted.
Measurable disease per RECIST v1.1
   Previously irradiated lesions can be considered as measurable disease only if progressive
   disease has been unequivocally documented at that site since radiation.
Tumor PD-L1 expression with a TIC ≥5%, as determined by the investigational VENTANA PD-L1 (SP263) CDx Assay and documented through central testing of a representative tumor tissue specimen, in either a previously obtained archival tumor tissue or tissue obtained from a biopsy at prescreening/screening. After approximately 66 patients (55% of total planned enrollment) are enrolled in either PD-L1 subgroup (PD-L1 low (TIC 5%-19%) and PD-L1 high (TIC ≥20%)), additional patients in that PD-L1 subgroup are not enrolled.
   Confirmed availability of representative tumor specimen in formalin-fixed paraffin-embedded (FFPE) blocks (preferred) or at least 20 slides containing unstained, freshly cut, serial sections along with an associated pathology report. If only 15-19 slides are available, the patient may still be eligible for the study, after Medical Monitor confirmation has been obtained.
   If archival tumor tissue is either unavailable or is determined to be unsuitable for required testing, tumor tissue must be obtained from a biopsy performed at prescreening/screening. A biopsy may also be performed at prescreening/screening if a patient's archival tissue test results do not meet eligibility criteria.
ECOG Performance Status of 0 or 1
Life expectancy ≥12 weeks
Adequate hematologic and end-organ function, defined by the following laboratory test results, obtained within 14 days prior to randomization:
ANC ≥1.5×109/L (1500/μL) without granulocyte colony-stimulating factor support
Lymphocyte count ≥0.5×109/L (500/μL)
Platelet count ≥100×109/L (100,000/μL) without transfusion
Hemoglobin 90 g/L (9 g/dL)
   Patients may be transfused or receive erythropoietic treatment as per local SOC to meet this criterion.
AST, ALT, and alkaline phosphatase (ALP) ≤2.5×upper limit of normal (ULN), with the following exceptions:
   Patients with documented liver metastases: AST and ALT ≤5×ULN
   Patients with documented liver or bone metastases: ALP ≤5×ULN
Total bilirubin 1.5×ULN with the following exception:
   Patients with known Gilbert disease: total bilirubin ≤3×ULN
Creatinine ≤1.5×ULN
Albumin ≥25 g/L (2.5 g/dL)
For patients not receiving therapeutic anticoagulation: INR and aPTT ≤1.5×ULN
For patients receiving therapeutic anticoagulation: stable anticoagulant
Negative HIV test at screening
Negative hepatitis B surface antigen (HBsAg) test at screening
Positive hepatitis B surface antibody (HBsAb) test at screening, or negative HBsAb at screening accompanied by either of the following:
Negative total hepatitis B core antibody (HBcAb)
Positive total HBcAb test followed by quantitative hepatitis B virus (HBV) DNA <500 IU/mL
   The HBV DNA test must be performed for patients who have a negative HBsAg test, a negative HBsAb test and a positive total HBcAb test.
Negative hepatitis C virus (HCV) antibody test at screening, or positive HCV antibody test followed by a negative HCV RNA test at screening
The HCV RNA test must be performed for patients who have a positive HCV antibody test.
For women of childbearing potential: agreement to remain abstinent (refrain from heterosexual intercourse) or use contraception as defined below:
   Women must remain abstinent or use contraceptive methods with a failure rate of <1% per year during the treatment period and for 5 months after the final dose of atezolizumab and for 90 days after the last dose of tiragolumab/placebo.
   A woman is considered to be of childbearing potential if she is postmenarchal, has not reached a postmenopausal state (≥12 continuous months of amenorrhea with no identified cause other than menopause), and is not permanently infertile due to surgery (i.e., removal of ovaries, fallopian tubes, and/or uterus) or another cause as determined by the investigator (e.g., Müllerian agenesis). The definition of childbearing potential may be adapted for alignment with local guidelines or regulations.
   Examples of contraceptive methods with a failure rate of <1% per year include bilateral tubal ligation, male sterilization, hormonal contraceptives that inhibit ovulation, hormone-releasing intrauterine devices, and copper intrauterine devices.
   The reliability of sexual abstinence should be evaluated in relation to the duration of the clinical trial and the preferred and usual lifestyle of the patient. Periodic abstinence (e.g., calendar, ovulation, symptothermal, or postovulation methods) and withdrawal are not adequate methods of contraception. If required per local guidelines or regulations, locally recognized adequate methods of contraception and information about the reliability of abstinence are described in the local Informed Consent Form.
For men: agreement to remain abstinent (refrain from heterosexual intercourse) or use a condom, and agreement to refrain from donating sperm, as defined below:
   With a female partner of childbearing potential or pregnant female partner, men must remain abstinent or use a condom during treatment with tiragolumab/placebo and for 90 days after the final dose of tiragolumab/placebo to avoid exposing the embryo. Men must refrain from donating sperm during this same period.
   The reliability of sexual abstinence should be evaluated in relation to the duration of the clinical trial and the preferred and usual lifestyle of the patient. Periodic abstinence (e.g., calendar, ovulation, symptothermal, or postovulation methods) and withdrawal are not adequate methods of preventing drug exposure. If required per local guidelines or regulations, information about the reliability of abstinence are described in the local Informed Consent Form.

E. Exclusion Criteria

Patients who meet any of the following criteria are excluded from study:
Disease suitable for local therapy with curative intent
Progressive or recurrent disease within 6 months of the last dose of curative intent systemic treatment for locally advanced SCCHN
Grade ≥2 unresolved toxicity related to surgery or other prior therapies
   Patients with ≤Grade 2 alopecia or neuropathy are permitted.
   Patients that received major surgery must have recovered sufficiently, including recovery from any complications related to the intervention.
   Patients with an irreversible toxicity that is managed and is not expected to be exacerbated by study drug treatment may be included after consultation with the Medical Monitor.
Symptomatic, untreated, or actively progressing central nervous system (CNS) metastases Asymptomatic patients with treated CNS lesions are eligible, provided that all of the following criteria are met:
   Measurable disease, per RECIST v1.1, must be present outside the CNS.
   The patient has no history of intracranial hemorrhage or spinal cord hemorrhage.
   The patient has not undergone stereotactic radiotherapy within 7 days prior to randomization, whole-brain radiotherapy within 14 days prior to randomization, or neurosurgical resection within 28 days prior to randomization.
   The patient has no ongoing requirement for corticosteroids as therapy for CNS disease.

Anticonvulsant therapy at a stable dose is permitted.
History of leptomeningeal disease
Uncontrolled tumor-related pain
   Patients requiring pain medication must be on a stable regimen at study entry.
   Symptomatic lesions (e.g., bone metastases or metastases causing nerve impingement) amenable to palliative radiotherapy should be treated prior to enrollment.
   Patients should be recovered from the effects of radiation. There is no required minimum recovery period.
   Asymptomatic metastatic lesions that would likely cause functional deficits or intractable pain with further growth (e.g., epidural metastasis that is not currently associated with spinal cord compression) should be considered for loco-regional therapy if appropriate prior to enrollment.
Uncontrolled or symptomatic hypercalcemia
Active or history of autoimmune disease or immune deficiency, including, but not limited to, myasthenia gravis, myositis, autoimmune hepatitis, systemic lupus erythematosus, rheumatoid arthritis, inflammatory bowel disease, antiphospholipid antibody syndrome, Wegener granulomatosis, Sjögren syndrome, Guillain-Barré syndrome, or multiple sclerosis with the following exceptions:
   Patients with a history of autoimmune-related hypothyroidism who are on thyroid-replacement hormone are eligible for the study.
   Patients with controlled Type 1 diabetes mellitus who are on an insulin regimen are eligible for the study.
   Patients with eczema, psoriasis, lichen simplex chronicus, or vitiligo with dermatologic manifestations only (e.g., patients with psoriatic arthritis are excluded) are eligible for the study provided all of following conditions are met:
      Rash must cover <10% of body surface area
      Disease is well controlled at baseline and requires only low-potency topical corticosteroids
      No occurrence of acute exacerbations of the underlying condition requiring psoralen plus ultraviolet A radiation, methotrexate, retinoids, biologic agents, oral calcineurin inhibitors, or high-potency or oral corticosteroids within the previous 12 months
History of idiopathic pulmonary fibrosis, organizing pneumonia (e.g., bronchiolitis obliterans), drug-induced pneumonitis, or idiopathic pneumonitis, or evidence of active pneumonitis on screening chest computed tomography (CT) scan
   History of radiation pneumonitis in the radiation field (fibrosis) is permitted.
Active tuberculosis
Significant cardiovascular disease (such as New York Heart Association Class II or greater cardiac disease, myocardial infarction, or cerebrovascular accident) within 3 months prior to initiation of study treatment, unstable arrhythmia, or unstable angina
Major surgical procedure, other than for diagnosis, within 4 weeks prior to initiation of study treatment, planned, or anticipated need for a major surgical procedure during the study
History of additional malignancy other than SCCHN within 5 years prior to randomization, with the exception of malignancies with a negligible risk of metastasis or death (e.g., 5-year OS rate >90%), such as adequately treated carcinoma in situ of the cervix, non-melanoma skin carcinoma, localized prostate cancer, ductal carcinoma in situ, or Stage I uterine cancer
Severe infection within 4 weeks prior to initiation of study treatment, including, but not limited to, hospitalization for complications of infection, bacteremia, or severe pneumonia
Treatment with therapeutic oral or IV antibiotics within 2 weeks prior to initiation of study treatment
   Patients receiving prophylactic antibiotics (e.g., to prevent a urinary tract infection or chronic obstructive pulmonary disease exacerbation) are eligible for the study.
Prior allogeneic stem cell or solid organ transplantation
Any other disease, metabolic dysfunction, physical examination finding, or clinical laboratory finding that contraindicates the use of an investigational drug, may affect the interpretation of the results, or may render the patient at high risk from treatment complications
Treatment with a live, attenuated vaccine within 4 weeks prior to initiation of study treatment, or anticipation of need for such a vaccine during study treatment, within 5 months after the final dose of atezolizumab, or within 90 days after the final dose of tiragolumab/placebo.
Current treatment with anti-viral therapy for HBV or HCV
Positive Epstein-Barr virus (EBV) viral capsid antigen (VCA) IgM test at screening
   An EBV polymerase chain reaction (PCR) test should be performed as clinically indicated to screen for acute infection or suspected chronic active infection. Patients with a positive EBV PCR test are excluded.
Treatment with investigational therapy within 28 days prior to initiation of study treatment
Prior treatment with CD137 agonists or immune checkpoint blockade therapies, including anti-CTLA-4, anti-TIGIT, anti-PD-L1, and anti-PD-1 therapeutic antibodies
Treatment with systemic immunostimulatory agents (including, but not limited to, interferon and interleukin 2 [IL-2]) within 4 weeks or 5 drug-elimination half-lives (whichever is longer) prior to initiation of study treatment
Treatment with systemic immunosuppressive medication (including, but not limited to, corticosteroids, cyclophosphamide, azathioprine, methotrexate, thalidomide, and anti-TNF-α agents) within 2 weeks prior to initiation of study treatment, or anticipation of need for systemic immunosuppressive medication during study treatment, with the following exceptions:
Patients who received acute, low-dose systemic immunosuppressant medication or a one-time pulse dose of systemic immunosuppressant medication (e.g., 48 hours of corticosteroids for a contrast allergy) are eligible for the study after Medical Monitor confirmation has been obtained.
Patients who receive mineralocorticoids (e.g., fludrocortisone), inhaled or low-dose systemic corticosteroids for chronic obstructive pulmonary disease or asthma, or low-dose corticosteroids for orthostatic hypotension or adrenal insufficiency are eligible for the study.
History of severe allergic anaphylactic reactions to chimeric or humanized antibodies or fusion proteins
Known hypersensitivity to Chinese hamster ovary cell products or to any component of the atezolizumab or tiragolumab formulation
Pregnancy or breastfeeding, or intention of becoming pregnant during study treatment, within 5 months after the final dose of atezolizumab, or within 90 days after the final dose of tiragolumab/placebo.
   Women of childbearing potential must have a negative serum pregnancy test result within 14 days prior to initiation of study treatment.

F. Assessments

Patients are closely monitored for safety and tolerability throughout the study. Patients are assessed for toxicity prior to each dose; dosing occurs only if the clinical assessment and local laboratory test values are acceptable.

Medical History, Baseline Conditions, Concomitant Medication, and Demographic Data

Medical history, including clinically significant diseases, surgeries, cancer history (including prior cancer therapies and procedures), reproductive status, smoking history, and use of alcohol and drugs of abuse, is recorded at baseline. In addition, all medications (e.g., prescription drugs, over-the-counter drugs, vaccines, herbal or homeopathic remedies, nutritional supplements) used by the patient within 7 days prior to initiation of study treatment are recorded. At the time of each follow-up physical examination, an interval medical history is obtained and any changes in medications and allergies are recorded.

Demographic data includes age, sex, and self-reported race/ethnicity.

ECOG Performance Status

Performance status is measured using the ECOG Performance Status Scale.

Physical Examinations

A complete physical examination must be performed at screening, and should include an evaluation of the head, eyes, ears, nose, and throat, and the cardiovascular, dermatologic, musculoskeletal, respiratory, gastrointestinal, genitourinary, and neurologic systems. Any abnormality identified at baseline should be recorded.

Limited, symptom-directed physical examinations should be performed at specified postbaseline visits and as clinically indicated. Changes from baseline abnormalities should be recorded in patient notes. New or worsened clinically significant abnormalities should be recorded as adverse events.

Physical Examinations

Vital signs include measurements of respiratory rate, pulse rate, systolic and diastolic blood pressure, and temperature. Abnormalities observed at baseline and, at subsequent visits, new or worsened clinically significant abnormalities are recorded.

Tumor and Response Evaluations

Screening assessments and subsequent tumor assessments must include CT scans (with oral or IV contrast) of the chest and abdomen and CT scans (with oral or IV contrast) or magnetic resonance imaging (MRI) (with contrast) of the head and neck (base of skull to clavicle). A CT scan with contrast of the pelvis should be performed as clinically indicated or as per local SOC at screening and subsequent response evaluations. If a CT scan with contrast is contraindicated (e.g., in patients with impaired renal clearance), a non-contrast CT scan of the chest may be performed, and MRI scans with contrast of the head and neck, abdomen, and pelvis (as applicable) must be performed. Further investigations, such as bone scans should also be performed if clinically indicated at baseline and at subsequent response evaluations.

A CT scan with contrast or MRI scan with contrast (if CT with contrast is contraindicated) of the brain should be done as clinically indicated to evaluate CNS metastasis at screening and at subsequent response evaluations. If a CT scan with contrast is performed and the presence of brain metastases is considered equivocal, an MRI scan of the head is required to confirm or refute the diagnosis of CNS metastases at baseline. Patients with active or untreated CNS metastases are not eligible for the study. Patients with a history of irradiated brain metastasis at screening are not required to undergo brain scans at subsequent response evaluations unless clinically indicated.

If a CT scan for tumor assessment is performed in a positron emission tomography (PET)/CT scanner, the CT acquisition must be consistent with the standards for a full-contrast diagnostic CT scan.

Tumor assessments performed as SOC prior to obtaining informed consent may be used rather than repeating tests, provided the scans are of diagnostic quality and are performed within 28 days of randomization.

The same radiographic modality (e.g., CT scan with contrast) and procedures (e.g., the same contrast protocol for CT scans) used at screening must be used for all subsequent tumor assessments. All known sites of disease, including measurable and/or non-measurable disease, must be documented at screening and re-assessed at each subsequent tumor evaluation.

Patients undergo tumor assessments at baseline, every 6 weeks (±7 days) for the first 30 weeks following Day 1 of Cycle 1, and every 9 weeks (±7 days) after completion of the Week 30 tumor assessment. Tumor assessments continue per schedule regardless of treatment delays until radiographic disease progression per RECIST v1.1 or loss of clinical benefit for patients who continue study treatment after radiographic disease progression per RECIST v1.1, withdrawal of consent, death, or study termination by the Sponsor, whichever occurs first. At the investigator's discretion, scans must also be performed at any time if progressive disease or loss of clinical benefit is suspected.

Patients who discontinue study treatment (for any reason, including, but not limited to clinical decline or toxicity) in the absence of radiographic disease progression per RECIST v.1.1 continue to undergo tumor response assessments per the schedule described above, regardless of whether a new anti-cancer therapy is started. Tumor assessments continue until radiographic disease progression per RECIST v1.1, withdrawal of consent, death, or study termination by the Sponsor, whichever occurs first Response is assessed by the investigator on the imaging modalities detailed above, using RECIST v1.1. The investigator's assessment of overall tumor response at all timepoints should only be based on RECIST v1.1. Assessments should be performed by the same evaluator, if possible, to ensure internal consistency across visits.

Investigator assessment of overall tumor response at all timepoints are only based on RECIST v1.1. Tumor assessments must be continued after disease progression per RECIST v1.1 for patients who receive study treatment beyond progression. This includes continued measurement of target lesions, evaluation of non-target lesions, and evaluation of any newly identified lesions at all subsequent assessments.

Laboratory, Biomarker, and Other Biological Samples

Samples for the following laboratory tests are sent to the study site's local laboratory for analysis:
Hematology: WBC count, RBC count, hemoglobin, hematocrit, platelet count, and differential count (neutrophils, eosinophils, basophils, monocytes, and lymphocytes)
Chemistry panel (serum or plasma): bicarbonate or total carbon dioxide (if considered SOC for the region), sodium, potassium, chloride, glucose, BUN or urea, creatinine, total protein, albumin, phosphate, calcium, total bilirubin, ALP, ALT, AST, and LDH
Coagulation: INR and aPTT
Thyroid function testing: thyroid-stimulating hormone, free triiodothyronine (T3) (or total T3 for sites where free T3 is not performed), and free thyroxine (also known as T4)
EBV serology, including the following
   EBV VCA IgM
   EBV VCA IgG or Epstein-Barr virus nuclear antigen (EBNA) IgG
   If clinically indicated: EBV PCR
HIV serology
HBV serology, consisting of the following:
   HBsAg, HBsAb, and total HBcAb for all patients
   HBV DNA for patients with negative HBsAg and HBsAb tests and a positive total HBcAb test
   Patients with a positive quantitative HBV DNA at screening (must be <500 IU/mL per the eligibility criteria) undergo additional HBV DNA tests as outlined in the schedule of activities.
HCV serology: HCV antibody and (if HCV antibody test is positive) HCV RNA
For patients with SCCHN involving the oropharynx: HPV status of tumor tissue determined by p16 IHC, in situ hybridization, or PCR-based assay
Pregnancy test
   All women of childbearing potential have a serum pregnancy test at screening. During study treatment urine pregnancy tests are performed on Day 1 of every cycle. Urine pregnancy test are also performed at the treatment discontinuation visit. If a urine pregnancy test is positive, it must be confirmed by a serum pregnancy test.
   A woman is considered to be of childbearing potential if she is postmenarchal, has not reached a postmenopausal state (≥12 continuous months of amenorrhea with no identified cause other than menopause), and is not permanently infertile due to surgery (i.e., removal of ovaries, fallopian tubes, and/or uterus) or another cause as determined by the investigator (e.g., Müllerian agenesis).
Urinalysis (pH, specific gravity, glucose, protein, ketones, and blood); dipstick permitted

The following samples are sent to one or several central laboratories or to the Sponsor or a designee for analysis:
Serum sample for analysis of autoantibodies: anti-nuclear antibody, anti-double-stranded DNA, circulating anti-neutrophil cytoplasmic antibody, and perinuclear anti-neutrophil cytoplasmic antibody
Serum sample for C-reactive protein
Serum samples for atezolizumab and tiragolumab PK analysis through use of a validated assay
Serum samples for assessment of ADAs to atezolizumab and tiragolumab through use of a validated assay
Blood and plasma samples for exploratory research on biomarkers
   Blood samples may be processed to obtain peripheral blood mononuclear cells (PBMCs) and other derivatives (e.g., RNA, DNA, etc.).
Archival or newly collected tumor tissue sample obtained at baseline for determination of PD-L1 expression and for exploratory research on biomarkers
   A representative FFPE tumor specimen in a paraffin block (preferred) or at least 20 slides containing unstained, freshly cut, serial sections must be submitted along with an associated pathology report prior to study enrollment. If only 15-19 slides are available, the patient may still be eligible for the study, after Medical Monitor approval has been obtained.
   Tumor tissue should be of good quality based on total and viable tumor content.
   Samples must contain a minimum of 50 viable TCs that preserve cellular context and tissue architecture regardless of needle gauge or retrieval method.
   Samples collected via resection, core-needle biopsy (at least three cores, embedded in a single paraffin block), or excisional, incisional, punch, or forceps biopsy are acceptable. Fine-needle aspiration (defined as samples that do not preserve tissue architecture and yield cell suspension and/or smears), brushing, cell pellets from pleural effusion, and lavage samples are not acceptable. Tumor tissue from bone metastases that have been decalcified is not acceptable. If archival tumor tissue is unavailable or is determined to be unsuitable for required testing, a pretreatment tumor biopsy is required. A pretreatment tumor biopsy may also be performed if a patient's archival tissue test results do not meet eligibility criteria.
Tumor tissue sample obtained at the time of progression, if deemed clinically feasible, for exploratory research on biomarkers
   Biopsies at the time of progression should be performed within 40 days after progression or prior to the next anti-cancer therapy, whichever is sooner.
   Samples collected via resection, core-needle biopsy (at least three cores preferred), or excisional, incisional, punch, or forceps biopsy are preferred.
   NGS may be performed by Foundation Medicine. If performed by Foundation Medicine, the investigator may obtain an NGS report through Foundation Medicine's web portal. If allowed by local laws, the investigator may share and discuss the results with the patient, unless the patient chooses otherwise. The NGS report is generated for research purposes and is not provided for the purpose of guiding future treatment decisions. Results are not be available for samples that do not meet criteria for testing.

Exploratory biomarker research may include, but are not limited to, analysis of tumor gene alterations, HPV status or gene signatures associated with tumor immunobiology, PD-L1, TIGIT, lymphocyte subpopulations, and T-cell receptor repertoire. Research may involve extraction of DNA, cell-free DNA, or RNA; analysis of mutations, and other tumor specific genomic variants; and genomic profiling through use of NGS of a comprehensive panel of genes. DNA extracted from blood may be compared with DNA extracted from tissue to identify somatic variants by distinguishing germline variants from somatic variants. NGS methods may include WGS or WES of tissue and blood samples, but WGS or WES of blood samples are performed only at participating sites.

For sampling procedures, storage conditions, and shipment instructions, see the laboratory manual.

Unless the patient gives specific consent for his or her leftover samples to be stored for optional exploratory research, biological samples are destroyed no later than the time of completion of the final Clinical Study Report, with the following exceptions:
Serum samples collected for PK or immunogenicity analysis may be needed for additional immunogenicity characterization and for PK or immunogenicity assay development and validation; therefore, these samples are destroyed no later than 5 years after the final Clinical Study Report has been completed.
Blood, plasma, and PBMC samples collected for biomarker research are destroyed no later than 5 years after the final Clinical Study Report has been completed, with the exception of tissue samples that undergo WGS or WES, which are stored until they are no longer needed or until they are exhausted. However, the storage period is in accordance with the IRB/EC-approved Informed Consent Form and applicable laws (e.g., health authority requirements).
For enrolled patients, remaining archival tissue blocks are returned to the site upon request, or no later than 18 months after the final Clinical Study Report, whichever occurs first. For patients who are not enrolled, remaining archival tissue blocks are returned to the site no later than 6 weeks after eligibility determination.

When a patient withdraws from the study, samples collected prior to the date of withdrawal may still be analyzed, unless the patient specifically requests that the samples be destroyed or local laws require destruction of the samples. However, if samples have been tested prior to withdrawal, results from those tests remain as part of the overall research data.

Given the complexity and exploratory nature of exploratory biomarker analyses, data derived from these analyses are generally not be provided to study investigators or patients unless required by law (with the exception of the report from Foundation Medicine, which is only applicable to biopsies obtained at disease progression). The aggregate results of any conducted research are available in accordance with the effective Sponsor policy on study data publication.

Electrocardiograms

An ECG is required at screening and as clinically indicated at other timepoints during the study. ECGs for each patient should be obtained from the same machine wherever possible. Lead placement should be as consistent as possible. ECG recordings must be performed after the patient has been resting in a supine position for at least 10 minutes.

For safety monitoring purposes, the investigator must review, sign, and date all ECG tracings. Copies of ECG tracings are kept as part of the patient's permanent study file at the site. Any morphologic waveform changes or other ECG abnormalities must be documented on the eCRF.

Clinical Outcome Assessments

PRO instruments are completed to more fully characterize the clinical profile of atezolizumab plus tiragolumab and atezolizumab plus placebo. In addition, PRO instruments enable the capture of each patient's direct experience with atezolizumab plus tiragolumab and atezolizumab plus placebo. PRO data are collected through use of the following instruments:
PROMIS® Item Bank v2.0-Physical Functioning-Short Form 10b
PROMIS® Item Bank v1.0-Fatigue-Short Form 4a
PROMIS® Item Bank v1.1-Pain Interference-Short Form 4a
PROMIS® Numeric Rating Scale v1.0-Pain Intensity 1 a
Patient Global Impression of Severity (PGI-S)
Patient Global Impression of Change and its Importance (PGI-CI)
Most Important Symptoms (MIS)
PRO-CTCAE

PRO questionnaires (i.e., PROMIS®, PGI-S/PGI-CI, MIS, and PRO-CTCAE) are completed during treatment and at the treatment discontinuation visit.

Data Collection Methods for Clinical Outcome Assessments

PRO instruments used to characterize the clinical profile of atezolizumab plus tiragolumab and atezolizumab plus placebo are self-administered or interviewer-administered (as appropriate) at the clinic at specified timepoints during the study. PRO data obtained through use of PROMIS®, PGI-S/PGI-CI, MIS, and PRO-CTCAE questionnaires.

At the clinic, instruments are administered before the patient receives any information on disease status, prior to the performance of non-PRO assessments, and prior to the administration of study treatment, unless otherwise specified.

Paper PRO questionnaires, translated into the local language as appropriate, scheduled for administration during a clinic visit must be completed by the patient at the investigational site at the start of the clinic visit prior to other study assessments and before administration of study treatment to avoid as much as possible, any assessment bias. Patients complete paper versions of the questionnaires, which are provided by site staff. Interviewer assessment is allowed but can only be conducted by a member of the clinic staff for patients who are unable to complete the measures on their own.

Study personnel should review all questionnaires for completeness before the patient leaves the investigational site.

During clinic visits, PRO instruments should be administered as outlined below:
Patients' health status should not be discussed prior to administration of the instruments.
Sites must administer the official version of each instrument, as provided by the Sponsor. Instruments must not be copied from the protocol.
Sites should allow sufficient time for patients to complete the instruments, estimated to be 15 minutes on Day 1 of Cycles 1 and 2, and less than 10 minutes for each subsequent cycle.
Sites should administer the instruments in a quiet area with minimal distractions and disruptions.
Patients should be instructed to answer questions to the best of their ability; there are no right or wrong answers.
Site staff should not interpret or explain questions, but may read questions verbatim upon request.
Patients should not obtain advice or help from others (e.g., family members or friends) when completing the instruments.
Site staff should review all completed instruments and should ask the patient to rectify any response that is not clearly marked in the appropriate location. If a response is missing, site staff should ask the patient to complete the item or confirm that the item was intentionally left blank.

Description of Clinical Outcome Assessment Instruments

PROMIS® Item Bank v2.0-Physical Functioning-Short Form 10b

PROMIS® Item Bank v2.0-Physical Functioning-Short Form 10b consists of 10 questions designed to measure self-reported capability of physical activities including the functioning of upper extremities (dexterity), lower extremities (walking or mobility), and central regions (neck, back), as well as instrumental activities of daily living. All questions are scored on a 1-5 scale, with a score of 5 equating to the patient's highest ability to function and a score of 1 equating to the patient's lowest ability to function. The recall period is 7 days.

PROMIS® Item Bank v1.0-Fatigue-Short Form 4a

PROMIS® Item Bank v1.0-Fatigue-Short Form 4a consists of four items designed to assess a range of self-reported fatigue symptoms and the patient's ability to execute daily activities. Items on the questionnaire inquire about the patient's level of fatigue experienced over the past seven days. The items are scored on a 1-5 scale, with a score of 5 equating to the highest level of fatigue experienced by the patient and the score of 1 equating to the lowest level of fatigue experienced by the patient. The recall period is 7 days.

PROMIS® Item Bank v1.1-Pain Interference-Short Form 4a

PROMIS® Item Bank v1.1-Pain Interference-Short Form-4a consists of four questions on a self-reported scale designed to assess the consequences/interference of pain on relevant aspects of the patient's life. All questions are scored on a 1-5 scale, with a score of 5 equating to the highest level of interference from pain and the score of 1 equating to the lowest level of interference from pain experienced by the patient. The recall period is 7 days.

PROMIS® Numeric Rating Scale v1.0-Pain Intensity 1a

PROMIS® Numeric Rating Scale v1.0-Pain Intensity 1 a consists of one question on a self-reported scale designed to assess the patient's average pain level.

The question is scored on a 1-10 scale, with a score of 10 equating to the “worst imaginable pain” and the score of 1 equating to “no pain”. The recall period is 7 days.

Patient Global Impression of Severity (PGI-S)

The Patient Global Impression of Severity (PGI-S) is a one-item, self-reported measure used to assess the patient's impression about the severity of their overall condition during the preceding 7 days. The PGI-S utilizes a 5-point response scale, with a score of 5 equating to “very severe” and the score of 1 equating to “none” (adapted from Guy et al. 1976).

Patient Global Impression of Change and its Importance (PGI-CI)

The Patient Global Impression of Change is a one-item, self-reported measure used to assess the patient's impression about changes to their condition compared with when they began the study. It utilizes a 7-point response scale, with a score of 7 equating to “very much worse” and a score of 1 equating to “very much improved” (adapted from Guy et al. 1976). In a subsequent question to the Patient Global Impression of Change, the patient is asked to indicate if the change experienced was important to them, the response options are “yes”, “no”, or “not applicable”, if the patient did not experience any change.

Most Important Symptoms (MIS)

The purpose of the Most Important Symptoms questionnaire is to identify which symptoms have been most troublesome to the patient in the past 7 days. The MIS is based on the importance rating scale from the University of Washington Quality of Life Questionnaire Scale 2.0 and later, with the addition of a number of symptoms related to the disease and/or treatment (Rogers et al. 2002). Patients are asked to pick up to 4 symptoms that have been the most troublesome in the past week.

PRO-CTCAE

The PRO-CTCAE is a validated item bank that is used to characterize the presence, frequency of occurrence, severity, and/or degree of interference with daily function of 78 patient-reportable symptomatic treatment toxicities (Basch et al. 2014; Dueck et al. 2015). The PRO-CTCAE contains 124 questions that are rated either dichotomously (for determination of presence vs. absence) or on a 5-point scale (for determination of frequency of occurrence, severity, and interference with daily function). Treatment toxicities can occur with observable signs (e.g., vomiting) or non-observable symptoms (e.g., nausea). The standard PRO-CTCAE recall period is the previous 7 days.

A subset of 11 symptoms deemed most applicable to the current treatments has been selected for this study. Symptoms have been selected on the basis of prior studies with atezolizumab plus tiragolumab (Rodriguez-Abreu et al. 2020), the symptomatic treatment toxicities related to immunotherapy as reported by patients and clinicians (Hansen et al. 2020), and recommendations on symptoms to measure in head and neck cancer (Cella et al. 2011; Chera et Ia. 2014)

Blood Samples for Whole Genome Sequencing or Whole Exome Sequencing

At participating sites, blood samples are collected for DNA extraction to enable WGS or WES to identify variants specifically occurred in tumors that are predictive of response to study drug, are associated with progression to a more severe disease state. DNA extracted from blood may be compared with DNA extracted from tissue to identify somatic variants by distinguishing germline variants from somatic variants. The samples may be sent to one or more laboratories for analysis.

Collection and submission of blood samples for WGS or WES is contingent upon the review and approval of the exploratory research by each site's IRB/EC and, if applicable, an appropriate regulatory body. If a site has not been granted approval for WGS or WES, this section of the protocol is not applicable at that site.

Genomics is increasingly informing researcher's understanding of disease pathobiology. WGS and WES provide a comprehensive characterization of the genome and exome, respectively, and, along with clinical data collected in this study, may increase the opportunity for developing new therapeutic approaches or new methods for monitoring efficacy and safety or predicting which patients are more likely to respond to a drug or develop adverse events. Data are analyzed in the context of this study but may also be explored in aggregate with data from other studies. The availability of a larger dataset assists in identification and characterization of important biomarkers and pathways to support future drug development.

For sampling procedures, storage conditions, and shipment instructions, see the laboratory manual.

Blood samples collected for WGS or WES are to be stored until they are no longer needed or until they are exhausted. However, the storage period is in accordance with the IRB/EC-approved Informed Consent Form and applicable laws (e.g., health authority requirements).

Samples for Research Biosample Repository

The Research Biosample Repository (RBR) is a centrally administered group of facilities used for the long-term storage of human biological specimens, including body fluids, solid tissues, and derivatives thereof (e.g., DNA, RNA, proteins, peptides). The collection, storage, and analysis of RBR samples facilitates the rational design of new pharmaceutical agents and the development of diagnostic tests, which may allow for individualized drug therapy for patients in the future.

Samples for the RBR are collected from patients who give specific consent to participate in this optional research. RBR samples are analyzed to achieve one or more of the following objectives:
To study the association of biomarkers with efficacy or disease progression
To identify safety biomarkers that are associated with susceptibility to developing adverse events or can lead to improved adverse event monitoring or investigation
To increase knowledge and understanding of disease biology and drug safety
To study drug response, including drug effects and the processes of drug absorption and disposition
To develop biomarker or diagnostic assays and establish the performance characteristics of these assays

Sample Collection

The following samples are stored in the RBR and used for research purposes, including, but not limited to, research on biomarkers related to atezolizumab, tiragolumab, head and neck cancer, or drug safety:
Leftover blood, plasma, PBMC, and tumor tissue samples (with the exception of remaining archival tissue blocks, which are returned to sites) and any derivatives thereof (e.g., DNA, RNA, proteins, peptides), including leftover tissue samples from medically indicated procedures (e.g., bronchoscopy, esophagogastroduodenoscopy, colonoscopy) performed at the investigator's discretion during the course of the study
Optional blood sample for DNA extraction collected at any time during the study
Optional stool sample collected predose at Cycle 1, Day 1 and between Cycle 2, Day 15 and predose at Cycle 3, Day 1. These samples may be collected at home.

The above samples may be sent to one or more laboratories for analysis of microbial communities through whole metagenomic sequencing, microbial culture, germline or somatic variants via WGS, WES, or other genomic analysis methods. Genomics is increasingly informing researcher's understanding of disease pathobiology. WGS and WES provide a comprehensive characterization of the genome and exome, respectively, and, along with clinical data collected in this study, may increase the opportunity for developing new therapeutic approaches or new methods for monitoring efficacy and safety or predicting which patients are more likely to respond to a drug or develop adverse events.

Data generated from RBR samples are analyzed in the context of this study but may also be explored in aggregate with data from other studies. The availability of a larger dataset assists in identification and characterization of important biomarkers and pathways to support future drug development.

RBR samples are to be stored until they are no longer needed or until they are exhausted. However, the RBR storage period is in accordance with the IRB/EC-approved Informed Consent Form and applicable laws (e.g., health authority requirements).

G. Treatment and Patient Discontinuation

Treatment Discontinuation

Patients must permanently discontinue study treatment if they experience any of the following:
Intolerable toxicity related to study treatment, including development of an immune-mediated adverse event determined by the investigator to be unacceptable given the individual patient's potential response to therapy and severity of the event
Any medical condition that may jeopardize the patient's safety if he or she continues study treatment
Investigator or Sponsor determination that treatment discontinuation is in the best interest of the patient
Use of another non-protocol anti-cancer therapy
Pregnancy
Radiographic disease progression per RECIST v1.1
   Exception: Patients are permitted to continue study treatment after RECIST v1.1 criteria for progressive disease are met, if they meet all of the criteria outlined for treatment beyond radiographic progression.
For patient treated beyond radiographic progression per RECIST v1.1: loss of clinical benefit as determined by the investigator after an integrated assessment of radiographic data, biopsy results (if available), and clinical status

The primary reason for study treatment discontinuation should be documented on the appropriate eCRF. Patients who discontinue study treatment prematurely are not replaced.

Patients return to the clinic for a treatment discontinuation visit 30 days after the final dose of study treatment. The visit at which response assessment shows progressive disease may be used as the treatment discontinuation visit, in which case all assessments associated with the treatment discontinuation visit should be performed at that time.

If a patient is discontinued from study treatment because of an adverse event (including adverse events of special interest) considered to be related to study treatment and the event is ongoing 30 days after the last dose of study treatment, the event must be followed until resolution or determination by the investigator that the event has become stable or irreversible.

Follow-up Assessments

Tumor Assessments

Patients who discontinue study treatment (for any reason, including, but not limited to clinical decline or toxicity) in the absence of radiographic disease progression per RECIST v.1.1 must continue to undergo tumor response assessments as outlined in the schedule of activities, regardless of whether a patient starts a new anti-cancer therapy, until radiographic disease progression per RECIST v1.1, withdrawal of consent, death, or study termination by the Sponsor, whichever occurs first. At the investigator's discretion, scans must be performed at any time if progressive disease is suspected.

Survival and Subsequent Therapies

After treatment discontinuation, information on survival follow-up and new anti-cancer therapy is collected by means of telephone calls, patient medical records, and/or clinic visits approximately every 3 months until death (unless the patient withdraws consent for survival follow-up or the Sponsor terminates the study).

Information on subsequent anti-cancer therapies include systemic therapies (e.g., chemotherapy, targeted therapy, immunotherapy), surgery (e.g., resection of local and/or metastatic disease), and radiation procedures (e.g., radiotherapy to a tumor lesion).

Patient Discontinuation

Patients have the right to voluntarily withdraw from the study at any time for any reason. In addition, the investigator has the right to withdraw a patient from the study at any time.

Reasons for patient discontinuation from the study may include, but are not limited to, the following:
Patient withdrawal of consent
Study termination or site closure
Loss to follow-up
Patient non-compliance, defined as failure to comply with protocol requirements as determined by the investigator or Sponsor

Every effort should be made to obtain a reason for patient discontinuation from the study. The primary reason for discontinuation from the study should be documented on the appropriate eCRF. If a patient requests to be withdrawn from the study (i.e. requests to withdrawn from all survival follow-up), this request must be documented in the source documents and signed by the investigator. Patients who withdraw from the study (i.e. survival follow-up) are not replaced.

If a patient withdraws from the study (i.e. survival follow-up), the study staff may use a public information source (e.g., county records) to obtain information about survival status.

H. Analysis

Efficacy Analysis

Primary Efficacy Endpoint

The primary efficacy endpoint is confirmed ORR. A confirmed objective response is defined as either a CR or a PR on 2 consecutive occasions ≥4 weeks apart, as determined by the investigator using RECIST v1.1. Patients who do not meet these criteria, including patients without any post-baseline tumor assessment, are considered non-responders. Confirmed ORR is defined as the proportion of patients who had a confirmed objective response.

The primary efficacy analysis takes place once all patients have been enrolled and a minimum follow-up of approximately 5 months has been achieved among those patients, who remain in follow-up for ORR assessment.

The primary efficacy analysis population consists of all randomized patients grouped according to their assigned treatment. An estimate of the ORR and its 95% CI is calculated using the Clopper-Pearson method for each treatment arm.

Secondary Efficacy Endpoints

Duration of Response

DOR is assessed in patients who have achieved a confirmed objective response, as determined by the investigator according to RECIST v1.1. DOR is defined as the time interval from the date of the first occurrence of a confirmed objective response until the first date of progressive disease, as determined by the investigator according to RECIST v1.1, or death from any cause, whichever occurs first. Patients who have not progressed and who have not died at the time of analysis are censored at the time of the last tumor assessment date. Kaplan-Meier methodology is used to estimate the median DOR for each treatment arm, and Kaplan-Meier curves are produced. The Brookmeyer-Crowley methodology is used to construct the 95% CI for the median DOR for each treatment arm.

Progression-Free Survival, Including Progression-Free Survival Rate at 6 Months

PFS is defined as the time between the date of randomization and the date of first documented disease progression, as assessed by investigators according to RECIST v1.1, or death, whichever occurs first. Patients who have not experienced disease progression or have not died by the data cutoff date are censored at the time of the last tumor assessment. Patients with no post-baseline tumor assessment are censored at the date of randomization.

Kaplan-Meier methodology is used to estimate the median PFS for each treatment arm, and Kaplan-Meier curves are produced. The Brookmeyer-Crowley methodology is used to construct the 95% CI for the median PFS for each treatment arm. The PFS rates at 6 months after randomization are estimated using Kaplan-Meier methodology for each treatment arm, along with 95% CIs calculated using the standard error derived from Greenwood's formula.

Overall Survival, Including Overall Survival Rate at 6 Months and 12 Months OS is defined as the time between the date of randomization and death from any cause. Data for patients who are not reported as having died by the data cutoff date are censored at the date when they were last known to be alive. Data for patients who do not have post-baseline information are censored at the date of randomization. OS and OS rate at 6 months and 12 months are analyzed using the same methods as described for PFS.

Patient Reported Outcomes

TTCD for physical functioning using the PROMIS® Item Bank v2.0-Physical Functioning-Short Form 10b is defined as the time from the date of randomization until the first confirmed clinically meaningful deterioration. Confirmed clinically meaningful deterioration in physical function is defined as a clinically meaningful decrease from baseline that must be held for at least two consecutive assessments, or an initial clinically meaningful increase above baseline followed by death. A T-score change >4 points is considered to be clinically meaningful for the physical functioning subscale score.

TTCD is assessed in ITT population. Patients who have not experienced a confirmed clinically meaningful deterioration at the clinical cutoff date are censored at the last time when they completed an assessment. If no baseline or post-baseline assessment is performed, patients are censored at the randomization date. TTCD using the PROMIS® scale is analyzed using the same methods as for PFS.

Exploratory analysis of the PROMIS® scales (pain, fatigue and physical functioning) includes summary statistics and the mean change from baseline in the ITT population as reported by the use of the PROMIS® pain, PROMIS® fatigue, and PROMIS® physical functioning questionnaires and calculated according to the PROMIS® scoring manuals.

Summary statistics, including counts and proportions, are presented for the PGI-CI, PGI-S and Most Important Symptoms and are exploratory.

Analysis of the PRO-CTCAE is exploratory in nature and primarily descriptive, with a focus on characterizing the pattern of disease and symptomatic treatment toxicities during the study. Results from these exploratory analyses are presented separately from the other safety analyses. PRO-CTCAE data are analyzed at the item level in line with current NCI recommendations for data handling. For each treatment arm, the number (percentage) of patients reporting symptom by “frequency,” “severity,” “interference,” and “presence” category are reported at each assessment.

In addition, the frequency of symptoms is cross-tabulated with the severity of the symptom to explore the impact of the symptom when it occurred. A summary table of the percentage of patients reporting severity of a symptom as “severe” or “very severe” during the study by treatment arm is also provided.

Safety Analysis

The safety analysis population consists of all randomized patients who received at least one dose of atezolizumab or tiragolumab/placebo.

Safety analyses are performed by treatment arm and are based on actual treatment received. Specifically, a patient is included in the atezolizumab plus tiragolumab arm in the safety analyses if the patient receives any amount of tiragolumab, regardless of the initial treatment assignment at randomization.

Drug exposure is summarized, including duration, dosage, and dose intensity.

Verbatim description of adverse events is mapped to the MedDRA thesaurus terms and graded according the NCI CTCAE v5.0, and severity for CRS is also graded by the investigator according to the ASTCT consensus grading scale. All adverse events are summarized by treatment arm and NCI CTCAE grade. CRS is also summarized by treatment arm and ASTCT consensus grade. In addition, serious adverse events, immune-mediated adverse events, and adverse events leading to study treatment discontinuation or interruption are summarized accordingly. Multiple occurrence of the same event is counted once at the maximum severity.

All deaths and causes of deaths are summarized by treatment arm.

Laboratory data with values outside of the normal ranges are identified. Additionally, selected laboratory data, including ADA results, are summarized.

Pharmacokinetic Analysis

PK samples of tiragolumab and atezolizumab are collected in this study. Tiragolumab and atezolizumab serum concentration data (minimum serum concentration and maximum serum concentration) are tabulated and summarized.

Descriptive statistics include means, medians, ranges, and standard deviations, as appropriate.

Additional PK analyses are conducted, as appropriate, based on the availability of data.

Immunogenicity Analysis

The immunogenicity analyses include patients with any ADA assessment, with patients grouped according to treatment received. The number and proportion of treatment-emergent ADA-positive patients and ADA-negative patients are summarized by treatment arm.

The relationship between ADA status and safety, efficacy, and PK endpoints may be analyzed and reported by means of descriptive statistics.

Biomarker Analysis

Biomarker analyses are exploratory. Descriptive statistics are used to summarize the biomarker subgroups and their relationship to efficacy endpoints; data may be analyzed both in the context of this study and in aggregate with data from other studies.

Interim Analysis

An efficacy interim analysis is planned and is reviewed by IMC when approximately 60 patients have been enrolled and have had the opportunity to have been followed-up for approximately at least 5 months.

### Example 16. A Phase Ib/II, Open-Label, Multicenter, Randomized Umbrella Study Evaluating the Efficacy and Safety of Multiple Immunotherapy-Based Treatments and Combinations in Patients with Muscle-Invasive Bladder Cancer (MIBC)

The time from diagnosis of muscle-invasive bladder cancer (MIBC) to cystectomy is typically 4-8 weeks, providing a window of opportunity to provide patients with neoadjuvant therapy to improve rates of recurrence and OS. The benefits of neoadjuvant chemotherapy are modest, with a 5% increase in overall survival (OS) in patients treated with neoadjuvant chemotherapy as compared to cystectomy alone (Vale et al., Eur Urol, 48: 202-205, 2005). Patients who achieve a pathological complete response (pCR) benefit the most from having received neoadjuvant cisplatin-based chemotherapy, with 80-90% alive at 5 years follow up; however, up to 50% of patients may have residual high-risk disease (pT2 or higher) at the time of surgery, with less than 50% survival at 5 years (Rosenblatt et al., Eur Urol, 61: 1229-1238, 2011; Bhindi et al., Eur Urol, 72: 660-664, 2017). Therefore, pCR could be a surrogate marker for efficacy and increased survival in neoadjuvant therapies for MIBC.

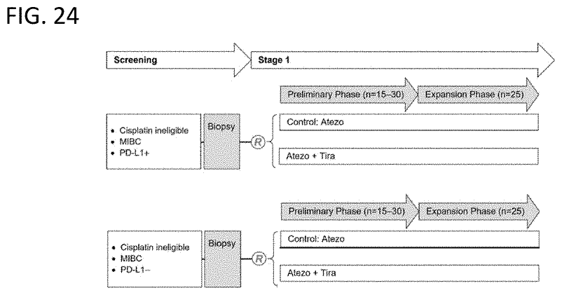

WO39613 is a Phase Ib/II, open-label, multicenter, randomized umbrella study evaluating the efficacy and safety of multiple immunotherapy-based treatments and combinations in patients with cisplatin-ineligible MIBC. The study is designed with the flexibility to open new treatment arms as new treatments become available, close existing treatment arms that demonstrate minimal clinical activity or unacceptable toxicity, or modify the patient population (e.g., with regard to prior anti-cancer treatment or biomarker status). Patients are randomly assigned to a control arm (atezolizumab (Atezo)) or an experimental arm consisting of atezolizumab in combination with tiragolumab (Atezo+Tira) (FIG. 24 and Table 69).

TABLE 69
Treatment regimens

| | | Number of patients | |
|---|---|---|---|
| MIBC cohort | Treatment^{a} | Preliminary phase | Expansion phase^{b} |
| Cohort 1 | Atezo (control) | Variable^{c} | Variable^{c} |
| PD-L1+ | Atezo + Tira | 30 | 25 |
| Cohort 2 | Atezo (control) | Variable^{c} | Variable^{c} |
| PD-L1− | Atezo + Tira | 30 | 25 |

Atezo: atezolizumab; MIBC: muscle invasive bladder cancer; Tira: tiragolumab.
^{a}The Sponsor may decide to delay or suspend enrollment within a given treatment arm. Thus, all experimental arms may not be open for enrollment at the same time.
^{b}If clinical activity is observed in an experimental arm during the preliminary phase, approximately 25 additional patients are enrolled in that arm during the expansion phase.
^{c}The randomization ratio depends on the number of experimental arms that are open for randomization (e.g., if an arm is added or randomization into an arm is suspended pending analysis of results from the preliminary phase), with the stipulation that the likelihood of being allocated to the control arm is no more than 35%.

The target and proposed mechanism-of-action classification for atezolizumab and tiragolumab are summarized in Table 70.

TABLE 70
Target and proposed mechanism-of-action classification
for atezolizumab and tiragolumab

| | Target(s) | Proposed mechanism-of-action classification |
|---|---|---|
| Atezolizumab | PD-L1 | Immune checkpoint inhibitor |
| Tiragolumab | TIGIT | TIGIT antagonist, improves the activation |
| | | and effectiveness of T-cell and NK-cell |
| | | tumor-killing activity |

PD-L1 = programmed death-ligand 1; TIGIT = T-cell immunoreceptor with Ig and ITIM domains.

Specific objectives and corresponding endpoints for the study are outlined in Table 71.

TABLE 71
Objectives and corresponding endpoints

| Primary Efficacy Objective | Corresponding Endpoint |
|---|---|
| To evaluate the efficacy of treatment in | pCR, defined as the proportion of patients |
| patients treated perioperatively for MIBC | with an absence of residual invasive cancer |
| | of the complete resected specimen |
| Secondary Efficacy Objective | Corresponding Endpoints |
| To evaluate the efficacy of treatment in | Landmark RFS, defined as RFS at specific |
| patients treated perioperatively for MIBC | timepoints (e.g., 12, 18, 24 months) |
| | Landmark EFS, defined as EFS at specific |
| | timepoints (e.g., 12, 18,24 months) |
| | Landmark OS, defined as OS at specific |
| | timepoints (e.g., 12, 18,24 months) |
| Exploratory Efficacy Objective | Corresponding Endpoints |
| To evaluate the efficacy of treatment in | RFS, defined as the time from Day 1 in the |
| patients treated perioperatively for MIBC | first cycle after surgery to the first documented |
| | recurrence of disease or death from any cause |
| | EFS, defined as the time from randomization to |
| | any of the following events (whichever occurs |
| | first): disease progression that precludes surgery, |
| | as assessed by the investigator according to |
| | RECIST v1.1; local or distant disease |
| | recurrence; or death from any cause |
| | OS, defined as the time from randomization to |
| | death from any cause |
| | Pathological downstaging rate, defined as the |
| | proportion of patients that reach ≤ pT1pN0 at |
| | the time of cystectomy |
| Safety Objective | Corresponding Endpoint |
| To evaluate the safety of | Incidence, nature, and severity of adverse events |
| treatment in patients treated | and laboratory abnormalities, with severity |
| perioperatively for MIBC | determined according to NCI CTCAE v4.0 |
| Exploratory Pharmacokinetic Objectives | Corresponding Endpoints |
| To characterize the PK profile of drugs | Plasma or serum concentration of each drug (as |
| that are administered to patients treated | appropriate) at specified timepoints |
| perioperatively for MIBC | |
| To evaluate potential relationships between | Relationship between plasma or serum |
| drug exposure and the efficacy and safety | concentration or PK parameters for each drug |
| of treatment | (as appropriate, on the basis of available data) |
| | and efficacy endpoints |
| | Relationship between plasma or serum |
| | concentration or PK parameters for each drug |
| | (as appropriate, on the basis of available data) |
| | and safety endpoints |
| Exploratory Immunogenicity | |
| Objectives | Corresponding Endpoint |
| To evaluate the immune response to drugs | For drugs for which ADA formation is measured: |
| that are administered to patients treated | presence of ADAs during the study relative to the |
| perioperatively for MIBC | presence of ADAs at baseline |
| To evaluate potential effects of ADAs in | For drugs for which ADA formation is measured: |
| patients treated perioperatively for MIBC | relationship between ADA status and efficacy, |
| | safety, or PK endpoints |
| Exploratory Biomarker | |
| Objective | Corresponding Endpoint |
| To identify biomarkers that are predictive of | Relationship between biomarkers in blood and |
| response to study treatment (i.e., predictive | tumor tissue and efficacy, safety, PK, |
| biomarkers), are associated with progression | immunogenicity, or other biomarker endpoints |
| to a more severe disease state (i.e., prognostic | |
| biomarkers), are associated with resistance | |
| to study treatment, are associated with | |
| susceptibility to developing adverse events | |
| (i.e., safety biomarkers), can provide evidence | |
| of study treatment activity (i.e., pharmaco- | |
| dynamic biomarkers), or can increase the | |
| knowledge and understanding of disease | |
| biology | |

ADA = anti-drug antibody;
CR = complete response;
DOR = duration of response;
EFS = event-free survival;
iRECIST = immune RECIST;
MIBC = muscle-invasive bladder cancer;
NCI CTCAE v4.0 = National Cancer Institute Common Terminology Criteria for Adverse Events, Version 4.0;
OS = overall survival;
PK = pharmacokinetic;
RFS = recurrence-free survival.
Overall response at a single timepoint is assessed by the investigator using RECIST v1.1. Overall response per iRECIST is calculated programmatically by the Sponsor on the basis of investigator-assessed individual lesion data.

Approximately 150-350 patients are enrolled, split between MIBC Cohort 1 (PD-L1+) and MIBC Cohort 2 (PD-L1−). Enrollment within the experimental arms takes place in two phases: a preliminary phase, followed by an expansion phase. All treatment arms are the same for MIBC Cohort 1 (PD-L1+) and MIBC Cohort 2 (PD-L1−) during the preliminary phase. Patients are assigned to either MIBC Cohort 1 (PD-L1+) or MIBC Cohort 2 (PD-L1−) following centralized PD-L1 testing of transurethral resection of bladder tumor (TURBT) samples using the VENTANA PD-L1 (SP142) Assay. Patients with a PD-L1 immune cell (IC) score of 2 or 3 (IC2/3), corresponding to the presence of discernible PD-L1 staining of any intensity in tumor-infiltrating immune cells covering ≥5% of tumor area occupied by tumor cells, associated intratumoral, and contiguous peritumoral stroma) are assigned to MIBC Cohort 1 (PD-L1+). Patients with an IC score of 0 or 1 (IC0/1, corresponding to the presence of discernible PD-L1 staining of any intensity in tumor-infiltrating immune cells covering 55% of tumor area occupied by tumor cells, associated intratumoral, and contiguous peritumoral stroma) are assigned to MIBC Cohort 2 (PD-L1-).

During the preliminary phase, up to 30 patients are enrolled in the Atezo+Tira arm and approximately 15 patients are enrolled in all other arms for each of the MIBC PD-L1 cohorts. If clinical activity is observed in an experimental arm during the preliminary phase, approximately 25 additional patients are enrolled in that arm during the expansion phase.

The Sponsor may decide to delay or suspend enrollment within a given treatment arm. Experimental arms with insufficient clinical activity or unacceptable toxicity are not expanded. Additional patients may be enrolled to ensure balance among treatment arms with respect to demographic and baseline characteristics, including potential predictive biomarkers, in order to enable further subgroup analyses.

The randomization ratio depends on the number of experimental arms that are available (e.g., if an arm is added or enrollment in an arm is suspended, pending analysis of results from the preliminary phase), with the stipulation that the likelihood of being allocated to the control arm is no more than 35%. Randomization takes into account arm-specific exclusion criteria. Patients are ineligible for a specific arm if they meet any of the exclusion criteria outlined for that arm.

Patients in the atezolizumab control arm and the experimental arms are treated until unacceptable toxicity or loss of clinical benefit, as determined by the investigator after an integrated assessment of radiographic and biochemical data, local biopsy results (if available), and clinical status (e.g., symptomatic deterioration such as pain secondary to disease). Patients receive up to 1 year of treatment post-surgery, followed by 2 years of 3-monthly follow-up assessments.

A. Assessments and Monitoring

All patients are closely monitored for adverse events throughout the study, and adverse events are graded according to the National Cancer Institute Common Terminology Criteria for Adverse Events, Version 4.0 (NCI CTCAE v4.0). Patients undergo tumor assessments every 9 weeks (starting on Day 1 of Cycle 1) for the first 54 weeks and then every 12 weeks thereafter. Response is assessed by the investigator using RECIST v1.1 (Eisenhauer et al., Eur J Cancer, 45: 228-247, 2009)). If clinical activity is demonstrated in an experimental arm, the Sponsor may request that tumor assessment scans for that arm be submitted for evaluation by a central reading facility.

Baseline tumor tissue samples are collected from all patients, preferably by means of a biopsy performed at study entry. If a biopsy is not deemed feasible by the investigator, archival tumor tissue may be submitted. Tumor tissue is also collected from patients who discontinue Stage 1 because of unacceptable toxicity or loss of clinical benefit, as determined by the investigator (if deemed clinically feasible by the investigator). These samples, as well as blood samples collected during the study, are utilized for biomarker research.

To characterize the pharmacokinetic (PK) properties and/or immunogenicity of atezolizumab and other therapeutic agents, blood samples are obtained at various timepoints before and during study treatment administration.

B. End of Study and Length of Study

The end of the study is defined as the date when the last patient completes the last visit in both stages, including survival follow-up visits conducted by telephone or on-site visit.

The total length of the study, from screening of the first patient to the end of the study, will be approximately 3-5 years.

C. Rationale for Study Design

Study Rationale and Benefit-Risk Assessment

The study is designed to accelerate the development of cancer immunotherapy (CIT) combinations by identifying early signals and establishing proof-of-concept clinical data in patients with cisplatin-ineligible MIBC.

Platinum-based neoadjuvant chemotherapy is associated with an improvement in OS, but is only suitable for a minority of patients. Given the relatively limited treatment options for patients with cisplatin-ineligible MIBC and additionally the poor prognosis and potential toxicities associated with treatments for locally advanced or metastatic UCs, these populations are in need of treatment options and are therefore considered appropriate for trials of novel therapeutic candidates. The currently prevailing CIT approach is to circumvent immune evasion mechanisms and reinvigorate anti-tumor responses by targeting T-cell inhibitory factors, such as programmed death-ligand 1 (PD-L1)/programmed death-1 (PD-1).

CIT has demonstrated clear clinical efficacy, with significant survival benefit observed across multiple advanced malignancies, and there is evidence that PD-1/PD-L1 checkpoint blockade can generate pCR and durable responses in patients with UC (Powles et al., Nat Med, 25: 1706-1714, 2019; Necchi et al., Int J Clin Oncol, 36: 3353-3360, 2018). Although these targets have resulted in remarkable clinical therapeutic success for various cancer indications, ongoing research indicates that a series of stepwise events is necessary for the generation of an effective anti-tumor immune response (Chen and Mellman, Immunity, 39: 1-10, 2013). Each event is critical for an effective response, and each is also susceptible to several tumor immune-evasion mechanisms. Thus, the need to identify and circumvent the various factors that account for the absence of, or escape from, an effective anti-cancer immune response will be critical for propagating cancer immunity and advancing the field of CIT. The combination of atezolizumab with agents targeting different immune-evasion mechanisms or novel second-generation CPIs may increase response rates and decrease rates of disease recurrence in this population further.

The potential risks for patients with MIBC are related to the adverse events associated with study treatments. In most countries, patients wait an average of 4-8 weeks between establishing the diagnosis of urothelial cancer and surgery. Participation in this trial that includes preoperative treatment is not expected to result in relevant delays of surgery for participants. Furthermore, atezolizumab did not have an impact on operability or increase the risks associated with surgery in the ABACUS study (Powles et al., Nat Med, 25: 1706-1714, 2019).

Rationale for Perioperative Treatment in Muscle Invasive Bladder Cancer

The therapeutic contribution of maintenance therapy following neoadjuvant therapy and surgery is unknown. The WO39613 study is designed to assess treatment efficacy and to perform extensive biomarker analyses on sequential patient tissues.

IMVigor010 is a Phase III, randomized study that randomly assigned patients with MIBC to receive atezolizumab versus observation as an adjuvant therapy following surgical resection and lymph node dissection. Although the study did not meet its primary endpoint of showing a disease-free survival benefit with atezolizumab adjuvant therapy in the intent-to-treat population, a subgroup analysis of T3 and T4 patients, as well as nonclinical data (Liu et al., Cancer Discov, 6: 1382-1399, 2016; Pai et al., Immunity, 50: 477-492, 2019; Hussain et al., J Clin Oncol, 38(15 Suppl.): 5000, 2020), suggest that immunotherapy may require that patients have residual or active disease for checkpoint immunotherapy to be effective.

D. Evidence for Immunotherapy in Patients with Muscle Invasive Bladder Cancer Who are Ineligible for Cisplatin-Based Chemotherapy

Approximately 50% of patients with MIBC are ineligible to receive cisplatin-based neoadjuvant chemotherapy, and there are currently no perioperative systemic treatments recommended for these patients; cystectomy with bilateral pelvic lymph node dissection is the standard of care. Furthermore, less than half of patients with MIBC who are eligible for neoadjuvant chemotherapy will receive it due to personal preference or other co-morbidities. Therefore, new therapies are needed to improve outcomes for patients with MIBC.

PD-1/PD-L1 checkpoint immunotherapy is approved for all patients who have progressed on chemotherapy for mUC and for patients with 1 L mUC who are either ineligible for platinum-based chemotherapy or are ineligible for cisplatin-based chemotherapy if their tumor is PD-L1+. The largest studies investigating the safety and efficacy of PD-1/PD-L1 checkpoint neoadjuvant immunotherapy in patients with MIBC were the ABACUS and PURE-01 studies (Powles et al., Nat Med, 25:1706-1714, 2019; Necchi et al., Int J Clin Oncol, 36: 3353-3360, 2018).

E. Rationale for Pathological Complete Remission as a Primary Endpoint

Pathological complete remission (pCR), as well as pathological downstaging (an exploratory endpoint), has been validated as a surrogate marker of activity in this setting due to its association with durable remission free survival (Rosenblatt et al., Eur Urol, 61: 1229-1238, 2011; Bhindi et al., Eur Urol, 72: 660-664, 2017).

Increased baseline CD8 T cell infiltration and PD-L1+ disease have been shown to increase the probability of a pCR with immune therapy in the neoadjuvant setting (Powles et al., Nat Med, 25: 1706-1714, 2019).

F. Rationale for Using Atezolizumab as the Comparator

No standard of care exists for providing systemic therapy before or after surgical resection for patients with MIBC who decline or are ineligible for cisplatin-based neoadjuvant chemotherapy.

Atezolizumab was chosen as a comparator treatment because the ABACUS and PURE-01 studies have shown that PD-1/PD-L1 checkpoint immunotherapy is active and safe in patients with MIBC, with response rates comparable to those historically observed with cisplatin-based chemotherapy. Based on the ABACUS and PURE-01 data, it is possible that compared to mUC, MIBC is typically more inflamed, and therefore more likely to benefit from PD-1/PD-L1 checkpoint immunotherapy, because PD-L1 expression was observed in only 25% and 28.5% of patients treated for mUC in the IMVigor211 and KN-045 studies, respectively.

Randomized studies evaluating immunotherapy combinations will allow identification of safe and effective treatments for patients with MIBC who are ineligible for cisplatin-based chemotherapy, while also significantly increasing understanding of cancer biology and improving the ability to iterate when designing new trials and treatments for all patients with MIBC. The study may also include possible future arms and cohorts using immunotherapy combinations that have demonstrated adequate clinical safety.

G. Rationale for Separate PD-L1+ and PD-L1− Cohorts

PD-1/PD-L1 blockade is approved by the FDA for 2L mUC and for patients with PD-L1+ mUC who are not eligible to receive cisplatin-based chemotherapy, but not for patients with MIBC. Although the pCR benefit in the ABACUS study was greater in atezolizumab-treated patients with PD-L1+(37.1%; n=13/35) than with PD-L1− (24.5%; n=12/49) disease, some patients with PD-L1− disease may still benefit from atezolizumab or immunotherapy combinations. The design of the study allows the Sponsor to separately evaluate the efficacy of atezolizumab and other immunotherapy combinations in these patient subsets and to close enrollment into arms in each cohort where there is no evidence of benefit, without affecting investigations in the other cohort.

H. Rationale for Investigations into the Impact of Perioperative Therapy on ctDNA for the Muscle Invasive Bladder Cancer Cohorts

Change in circulating tumor DNA (ctDNA) concentrations over time has potential for being used to detect disease recurrence (Moding et al., Nat Cancer, 1: 176-183, 2020). ctDNA shed from cancer cells into the peripheral blood can be collected non-invasively and tested for the presence of tumor-specific mutations. Several characteristics of UC make it a strong candidate for prognostication of disease recurrence utilizing ctDNA testing:
UC carries a high somatic mutation rate; therefore, very small numbers of genes or targeted regions can be highly informative to predict disease characteristics (Todenhöfer et al., Bladder Cancer, 4: 1929, 2018).
Metastatic recurrence in UC has been observed to have a clonal origin, not differing significantly in mutational analysis from the original tumor (Thomsen et al., Sci Rep, 7: 11702, 2017).
Analyses of paired tumors in UC identified few subclones, bolstering the prognostic potential of longitudinal ctDNA testing after definitive therapy (Lamy et al., Cancer Res, 76: 5894, 2016; Nodentoft et al., Cell Rep, 7: 1649-1 663, 2014).
An analysis was performed using the Natera Signatera ctDNA assay on samples taken longitudinally from 68 patients with MIBC who received neoadjuvant chemotherapy and underwent cystectomy. This analysis found that patients whose samples were ctDNA-positive at any timepoint post-cystectomy had an overall recurrence rate of 76% versus 0% for patients that maintained ctDNA negativity post-cystectomy (median follow-up of 21 months) (Christiensen et al. J Clin Oncol. 37(18): 1547-1557, 2019).

These data present a biomarker selected sub-population of MIBC patients with a high unmet need for urgent treatment options. However, there are currently no data on 1) the impact of neoadjuvant immunotherapy on ctDNA pre- and post-cystectomy in patients with urothelial cancer; 2) if maintenance immunotherapy can help prevent the recurrence of urothelial cancer following cystectomy in patients treated with neoadjuvant immunotherapy; 3) if maintenance immunotherapy would decrease the risk of disease recurrence if patients treated with neoadjuvant immunotherapy had measurable ctDNA following cystectomy.

As part of this study, ctDNA is measured retrospectively in all patients with PD-L1+MIBC treated with atezolizumab and with atezolizumab plus tiragolumab at 4 time points: 1) before Day 1 of Cycle 1; 2) before cystectomy; 3) 4-6 weeks post-cystectomy; and 4) 6 months post-cystectomy. Association between ctDNA results at these time points with event-free survival (EFS) and OS is investigated. These results will inform the design of future studies on the impact of neoadjuvant, as well as the value of maintenance therapy, in patients receiving checkpoint immunotherapy. ctDNAs investigated at these time points in other arms of the study pending the outcomes of these investigations and clinical activity observed in other arms.

This design, and the use of ctDNA, allow the evaluation of 1) the impact of neoadjuvant therapy on ctDNA and pathologic response; 2) the impact of surgery on residual ctDNA following neoadjuvant immunotherapy; and 3) whether maintenance immunotherapy reduces the risk of ctDNA and disease recurrence in patients with MIBC following surgery.

### Example 17. Materials and Methods for WO39613 Study

A. Inclusion Criteria

Patients must meet all of the following criteria:
Age ≥18 years.
Ability to comply with the protocol, in the investigator's judgment.
Eastern Cooperative Oncology Group (ECOG) performance status (PS) of 0 or 1.
Patients who refuse neoadjuvant cisplatin-based chemotherapy or in whom neoadjuvant cisplatin-based therapy is not appropriate. Cisplatin ineligibility for this study is defined by a creatinine clearance <60 mL/min and/or grade ≥2 hearing loss and/or grade ≥2 neuropathy.
Fit and planned-for cystectomy.
Histologically documented MIBC (pT2-4, N0, M0), also termed TCC or urothelial cell carcinoma of the urinary bladder.
   Residual disease after TURBT (surgical opinion, cystoscopy/endoscopy or radiological presence).
   Patients with mixed histologies are required to have a dominant transitional cell pattern.
Availability of a TURBT specimen that is suitable for determination of PD-L1 and additional biomarker status by means of central testing.
   TURBT specimen must be submitted along with an associated pathology report. Tumor tissue is processed by two different methods (i.e., paraffin-fixed tissue and fresh tissue are required for separate biomarker analyses). Tumor tissue of bladder or upper tract should be of good quality based on total and viable tumor content and must contain a muscle invasive component (i.e., T2 or greater) of the tumor as verified by local pathology review.
   Patients having additional tissue samples from procedures performed at different times in the course of their MIBC (e.g., a specimen from a prior TURBT) will be requested (but not required) to also submit these samples for central testing.
N0 or M0 disease by CT or MRI (within 4 weeks of registration).
Adequate hematologic and end-organ function, defined by the following laboratory test results, obtained within 14 days prior to initiation of study treatment:
   Absolute neutrophil count (ANC) ≥1.5×10⁹/L (1500/μL) without G-CSF support
   White blood cell (WBC) count ≥2.5×10⁹/L (2500/μL)
   Lymphocyte count ≥0.5×10⁹/L (500/μL)
   Platelet count ≥100×10⁹/L (100,000/μL) without transfusion
   Hemoglobin ≥90 g/L (9.0 g/dL)
   Patients may be transfused; however, transfusions are not allowed within 2 weeks of screening laboratory tests for eligibility.
   Patients with a solitary kidney or chronic kidney disease with low erythropoietin production may use erythropoietin-stimulating agents.
      AST, ALT, and alkaline phosphatase (ALP) ≤2.5×upper limit of normal (ULN)
      Bilirubin ≤1.5×ULN, with the following exception:
         Patients with known Gilbert disease: bilirubin level ≤3×ULN
      Albumin 25 g/L (2.5 g/dL)
      Creatinine clearance ≥30 mL/min (calculated using the Cockcroft-Gault formula)
      For patients not receiving therapeutic anticoagulation:
         Partial thromboplastin time (PTT) or aPTT≤1.5×ULN
         Prothrombin time (PT) or international normalized ratio (INR)≤1.5×ULN
Negative HIV test at screening.
   Patients without a prior positive HIV test result will undergo an HIV test at screening, unless not permitted per local regulations.
Negative total hepatitis B core antibody (HBcAb) test at screening, or positive total HBcAb test followed by quantitative hepatitis B virus (HBV) DNA <500 IU/mL at screening
Negative hepatitis C virus (HCV) antibody test at screening, or positive HCV antibody test followed by a negative HCV RNA test at screening.
For women of childbearing potential: agreement to remain abstinent (refrain from heterosexual intercourse) or use contraceptive measures and agreement to refrain from donating eggs.
For men: agreement to remain abstinent (refrain from heterosexual intercourse) or use contraceptive measures, and agreement to refrain from donating sperm.

B. Exclusion Criteria

Patients who meet any of the following criteria are excluded. Event grades in the exclusion criteria are based on NCI CTCAE v4.0.
Prior treatment with systemic immunostimulatory agents (including, but not limited to, agents targeting CTLA-4, interferon and interleukin 2) prior to the initiation of study treatment
Eligibility only for the control arm
Prior allogeneic stem cell or solid organ transplantation
Treatment with systemic immunosuppressive medication (including, but not limited to, corticosteroids, cyclophosphamide, azathioprine, methotrexate, thalidomide, and anti-tumor necrosis factor-α agents) within 2 weeks prior to initiation of study treatment, or anticipation of need for systemic immunosuppressant medication during study treatment, with the following exceptions:
   Patients who received acute, low-dose, systemic immunosuppressant medications, or a one-time pulse dose of systemic immunosuppressant medication (e.g., 48 hours of corticosteroids for a contrast allergy) are eligible for the study.
   Patients who received mineralocorticoids (e.g., fludrocortisone), corticosteroids for chronic obstructive pulmonary disease or asthma, or low-dose corticosteroids for orthostatic hypotension or adrenal insufficiency are eligible for the study.
Treatment with a live, attenuated vaccine within 4 weeks prior to initiation of study treatment, or anticipation of need for such a vaccine during atezolizumab treatment or within 5 months after the last dose of atezolizumab.
Uncontrolled tumor-related pain. Patients requiring pain medication must be on a stable regimen at study entry.
Active or history of autoimmune disease or immune deficiency, including, but not limited to, myasthenia gravis, myositis, autoimmune hepatitis, systemic lupus erythematosus, rheumatoid arthritis, inflammatory bowel disease, antiphospholipid antibody syndrome, Wegener granulomatosis, Sjögren syndrome, Guillain-Barré syndrome, or multiple sclerosis, with the following exceptions:

Patients with a history of autoimmune-related hypothyroidism who are on thyroid-replacement hormone are eligible for the study.

Patients with controlled Type 1 diabetes mellitus who are on a stable insulin regimen are eligible for the study.

Patients with eczema, psoriasis, lichen simplex chronicus, or vitiligo with dermatologic manifestations only (e.g., patients with psoriatic arthritis are excluded) are eligible for the study provided all of following conditions are met:
Rash must cover <10% of body surface area.
Disease is well controlled at baseline and requires only low-potency topical corticosteroids.
There is no occurrence of acute exacerbations of the underlying condition requiring psoralen plus ultraviolet A radiation, methotrexate, retinoids, biologic agents, oral calcineurin inhibitors, or high-potency or oral corticosteroids within the previous 12 months.
History of idiopathic pulmonary fibrosis, organizing pneumonia (e.g., bronchiolitis obliterans), drug-induced pneumonitis, or idiopathic pneumonitis, or evidence of active pneumonitis on screening chest computed tomography (CT) scan.
History of malignancy other than UC within 2 years prior to screening, with the exception of malignancies with a negligible risk of metastasis or death (e.g., 5-year OS rate >90%), such as adequately treated carcinoma in situ of the cervix, non-melanoma skin carcinoma, localized prostate cancer, ductal carcinoma in situ, or Stage I uterine cancer. Patients with localized prostate cancer (defined as Stage ≤pT2c, Gleason score ≤7, and prostate-specific antigen (PSA) at prostate cancer diagnosis ≤20 ng/mL) treated with curative intent and without PSA recurrence are eligible. Patients with preexisting low-risk prostate cancer (defined as Stage cT1/T2a, Gleason score ≤6, and PSA ≤10 ng/mL) who are treatment-naive and undergoing active surveillance are eligible.
Active tuberculosis (TB) (i.e., has signs and symptoms of TB).
Severe infection within 4 weeks prior to initiation of study treatment, including, but not limited to, hospitalization for complications of infection, bacteremia, or severe pneumonia, or any active infection that, in the opinion of the investigator, could impact patient safety.
Treatment with therapeutic oral or IV antibiotics within 2 weeks prior to initiation of study treatment. Patients receiving prophylactic antibiotics (e.g., to prevent a urinary tract infection or chronic obstructive pulmonary disease exacerbation) are eligible for the study.
Significant cardiovascular disease such as New York Heart Association cardiac disease (Class III or greater), myocardial infarction or cerebrovascular accident within 3 months prior to randomization, unstable arrhythmia, or unstable angina.
Uncontrolled hypertension (defined as systolic blood pressure >140 mmHg and/or diastolic blood pressure >95 mmHg). Anti-hypertensive therapy to achieve these parameters is allowed.
Grade ≥3 hemorrhage or bleeding event within 28 days prior to initiation of study treatment.
Major surgical procedure, other than for diagnosis, within 4 weeks prior to initiation of study treatment, or anticipation of need for a major surgical procedure other than cystectomy or nephroureterectomy, during the study. Placement of central venous access catheter (e.g., port or similar) is not considered a major surgical procedure and is therefore permitted.
Any other disease, metabolic dysfunction, physical examination finding, or clinical laboratory finding that contraindicates the use of an investigational drug, may affect the interpretation of the results, impair the ability of the patient to participate in the study, or may render the patient at high risk from treatment complications.
History of severe allergic reactions to chimeric or humanized antibodies or fusion proteins.
Known hypersensitivity to Chinese hamster ovary cell products or recombinant human antibodies.
Known intolerance or hypersensitivity to any of the study drugs or their excipients (including histidine, trehalose dihydrate, and polysorbate 20).
Known intolerance to any of the drugs required for premedication (acetaminophen, ranitidine, diphenhydramine, and methylprednisolone).
Pregnancy or breastfeeding, or intention of becoming pregnant during the study. Women of childbearing potential must have a negative serum pregnancy test result within 14 days prior to initiation of study treatment.

Additional Exclusion Criteria for Atezolizumab+Tiragolumab Arm

Patients who meet any of the following criteria are excluded from the atezolizumab+tiragolumab arm:
Active Epstein-Barr virus (EBV) infection or known or suspected chronic active EBV infection at screening

Patients with a positive EBV viral capsid antigen (VCA) IgM test at screening are excluded from this arm. An EBV polymerase chain reaction (PCR) test should be performed as clinically indicated to screen for active infection or suspected chronic active infection. Patients with a positive EBV PCR test are excluded from this arm.

C. Permitted Therapy

Patients are permitted to use the following therapies during the study:
Oral contraceptives
Hormone-replacement therapy
Prophylactic or therapeutic anticoagulation therapy (such as warfarin at a stable dose or low-molecular-weight heparin at a stable dose)
Prophylactic antibiotic or anti-viral treatment administered according to institutional standards
Inactivated influenza vaccinations
Megestrol acetate administered as an appetite stimulant
Mineralocorticoids (e.g., fludrocortisone)
Corticosteroids administered for chronic obstructive pulmonary disease or asthma
Low-dose corticosteroids administered for orthostatic hypotension or adrenocortical insufficiency
Hormonal therapy with gonadotropin-releasing hormone agonists or antagonists for prostate cancer
Palliative radiotherapy (e.g., treatment of known bony metastases or symptomatic relief of pain) as outlined below:

After 2 cycles of treatment, palliative radiotherapy is permitted, provided it does not interfere with the assessment of tumor target lesions (e.g., the lesion to be irradiated must not be the only site of measurable disease). Treatment with atezolizumab may be continued during palliative radiotherapy.
Local therapy (e.g., surgery, stereotactic radiosurgery, radiotherapy, radiofrequency ablation) as outlined below:

Patients experiencing a mixed response requiring local therapy for control of three or fewer lesions may still be eligible to continue study treatment after Medical Monitor approval has been obtained. Patients who receive local therapy directed at a target lesion are no longer evaluable for radiographic response but will remain evaluable for progression.

Premedication with antihistamines, antipyretics, and/or analgesics may be administered for the second and subsequent atezolizumab infusions only, at the discretion of the investigator.

Patients who experience infusion-associated symptoms may be treated symptomatically with acetaminophen, ibuprofen, diphenhydramine, and/or H2-receptor antagonists (e.g., famotidine, cimetidine), or equivalent medications per local standard practice. Serious infusion-associated events manifested by dyspnea, hypotension, wheezing, bronchospasm, tachycardia, reduced oxygen saturation, or respiratory distress should be managed with supportive therapies as clinically indicated (e.g., supplemental oxygen and 32-adrenergic agonists).

D. Materials and Methods for Atezolizumab Arm

Atezolizumab is administered at a fixed dose of 1200 mg every 3 weeks (Q3W) (1200 mg on Day 1 of each 21-day cycle) (Table 72), which is the approved dosage (TECENTRIQ® U.S. Package Insert; TECENTRIQ® SmPC). Anti-tumor activity has been observed across doses ranging from 1 to 20 mg/kg Q3W. In Study PCD4989g, the maximum tolerated dose of atezolizumab was not reached, and no dose-limiting toxicities were observed at any dose. The fixed dose of 1200 mg Q3W (equivalent to an average body weight-based dose of 15 mg/kg Q3W) was selected on the basis of both nonclinical studies (Deng et al., MAbs, 8: 593-603, 2016) and available clinical pharmacokinetic, efficacy, and safety data.

Patients in the atezolizumab control arm receive treatment until unacceptable toxicity or loss of clinical benefit as determined by the investigator after an integrated assessment of radiographic and biochemical data, local biopsy results (if available), and clinical status (e.g., symptomatic deterioration such as pain secondary to disease). Treatment must be initiated no later than 7 days after treatment assignment. Patients undergo surgery after Cycle 3; treatment in Cycle 4 should be re-initiated 4-6 weeks after surgery.

TABLE 72
Treatment regimen for atezolizumab arm

| Cycle length | Dose, route, and regimen |
|---|---|
| 21 days | Atezolizumab 1200 mg IV on Day 1 of each cycle |

Administration of atezolizumab will be performed in a monitored setting where there is immediate access to trained personnel and adequate equipment and medicine to manage potentially serious reactions.

Atezolizumab infusions are administered per the instructions outlined in Table 73.

TABLE 73
Administration of first and subsequent atezolizumab infusions

| First infusion | Subsequent infusions |
|---|---|
| No premedication is permitted prior to the | If the patient experienced an infusion-related |
| atezolizumab infusion, | reaction with any previous infusion, premedi- |
| Vital signs (pulse rate, respiratory rate, pulse | cation with antihistamines, antipyretics, and/or |
| oximetry, blood pressure, and temperature) | analgesics may be administered for subsequent |
| should be recorded within 60 minutes prior | doses at the discretion of the investigator. |
| to the infusion. | Vital signs should be recorded within 60 |
| Atezolizumab should be infused over | minutes prior to the infusion. |
| 60 (±15) minutes. | Atezolizumab should be infused over 30 (±10) |
| If clinically indicated, vital signs should be | minutes if the previous infusion was tolerated |
| recorded every 15 (±5) minutes during the | without an infusion-related reaction, or 60 (±15) |
| infusion and 30 (±10) minutes after the | minutes if the patient experienced an infusion- |
| infusion. | related reaction with the previous infusion. |
| Patients should be informed about | If the patient experienced an infusion-related |
| the possibility of delayed post-infusion | reaction with the previous infusion or if |
| symptoms and instructed to contact their | clinically indicated, vital signs should be |
| study physician if they develop such | recorded during the infusion and at 30 (±10) |
| symptoms. | minutes after the infusion. |

D. Materials and Methods for Atezolizumab+Tiragolumab Arm

Atezolizumab is administered as described above (see Table 73).

Tiragolumab is administered at a fixed dose of 600 mg IV Q3W (600 mg on Day 1 of each 21-day cycle). The fixed dose of 600 mg IV Q3W was selected on the basis of available clinical PK, efficacy, and safety data from the combined Phase Ia/Phase Ib Study GO301 03, with single-agent tiragolumab or tiragolumab in combination with atezolizumab. In the Phase Ia portion of the study with tiragolumab as a single agent, the MTD was not reached, and no DLTs were observed in dose escalation. As of the clinical cutoff date, anti-drug antibodies (ADAs) to tiragolumab were rare in the Phase Ia or Phase Ib portions across all dose levels. Prolonged stable disease was observed in patients in the Phase Ia portion of the study at tiragolumab doses beginning at 400 mg. In the Phase Ib portion of the study with tiragolumab plus atezolizumab, the MTD was not reached. Anti-tumor activity, as measured by radiographic partial responses, was observed across doses for tiragolumab beginning at 30 mg and ranging up to 600 mg in combination with 1200 mg atezolizumab.

Patients in the atezolizumab+tiragolumab arm receive treatment as outlined in Table 74 until unacceptable toxicity or loss of clinical benefit as determined by the investigator after an integrated assessment of radiographic and biochemical data, local biopsy results (if available), and clinical status (e.g., symptomatic deterioration such as pain secondary to disease). Patients undergo surgery after Cycle 3; treatment in Cycle 4 is re-initiated 4-6 weeks after surgery.

TABLE 74
Treatment regimen for
atezolizumab + tiragolumab arm

| Cycle | Dose, route, and regimen (drugs |
|---|---|
| length | listed in order of administration) |
| 21 days | Atezolizumab 1200 mg IV on |
| | Day 1 of each cycle |
| | Tiragolumab 600 mg IV on |
| | Day 1 of each cycle |

Tiragolumab is administered by IV infusion at a fixed dose of 600 mg on Day 1 of each 21-day cycle with a post-infusion observation period, as described in Table 75. Administration of tiragolumab is performed in a monitored setting where there is immediate access to trained personnel and adequate equipment and medicine to manage potentially serious reactions.

TABLE 75
Administration of first and subsequent tiragolumab infusions

| First infusion | Subsequent infusions |
|---|---|
| No premedication is permitted prior to the | If the patient experienced an infusion-related |
| tiragolumab infusion, | reaction with any previous infusion, premedication |
| Vital signs (pulse rate, respiratory rate, pulse | with antihistamines, antipyretics, and/or |
| oximetry, blood pressure, and temperature) | analgesics may be administered for subsequent |
| should be recorded within 60 minutes prior | doses at the discretion of the investigator. |
| to the infusion. | Vital signs should be recorded within 60 minutes |
| Tiragolumab should be infused over 60 (±10) | prior to the infusion. |
| minutes. | Tiragolumab should be infused over 30 (±10) |
| After the infusion of tiragolumab, the patient | minutes if the previous infusion was tolerated |
| begins a 60-minute observation period. | without an infusion-related reaction, or 60 (±10) |
| Record vital signs every 15 (±5) minutes | minutes if the patient experienced an infusion- |
| during the infusion and at 30 (±10) minutes | related reaction with the previous infusion. |
| after the infusion. | If the patient tolerated the previous infusion of |
| Patients are informed about the possibility of | tiragolumab well without infusion-associated |
| delayed post-infusion symptoms and are | adverse events, the observation period may be |
| instructed to contact their study physician | reduced to 30 minutes. |
| if they develop such symptoms. | If the patient experienced an infusion-related |
| | reaction with the previous infusion or if clinically |
| | indicated, vital signs should be recorded during |
| | the infusion and at 15 (±10) minutes after the |
| | infusion. |

### Example 18. Assessment of Safety for WO39613 Study

A. Safety Parameters and Definitions

Safety assessments consist of monitoring and recording adverse events, including serious adverse events and adverse events of special interest, performing protocol-specified safety laboratory assessments, measuring protocol-specified vital signs, and conducting other protocol-specified tests that are deemed critical to the safety evaluation of the study.

B. Adverse Events

According to the International Council for Harmonisation (ICH) guideline for Good Clinical Practice, an adverse event is any untoward medical occurrence in a clinical investigation subject administered a pharmaceutical product, regardless of causal attribution. An adverse event can therefore be any of the following:
Any unfavorable and unintended sign (including an abnormal laboratory finding), symptom, or disease temporally associated with the use of a medicinal product, whether or not considered related to the medicinal product.
Any new disease or exacerbation of an existing disease (a worsening in the character, frequency, or severity of a known condition).
Recurrence of an intermittent medical condition (e.g., headache) not present at baseline.
Any deterioration in a laboratory value or other clinical test (e.g., ECG, X-ray) that is associated with symptoms or leads to a change in study treatment or concomitant treatment or discontinuation from study treatment.
Adverse events that are related to a protocol-mandated intervention, including those that occur prior to assignment of study treatment (e.g., screening invasive procedures such as biopsies).

Serious Adverse Events

A serious adverse event is any adverse event that meets any of the following criteria:
Is fatal (i.e., the adverse event actually causes or leads to death).
Is life threatening (i.e., the adverse event, in the view of the investigator, places the patient at immediate risk of death). This does not include any adverse event that, had it occurred in a more severe form or was allowed to continue, might have caused death.
Requires or prolongs inpatient hospitalization.
Results in persistent or significant disability/incapacity (i.e., the adverse event results in substantial disruption of the patient's ability to conduct normal life functions).
Is a congenital anomaly/birth defect in a neonate/infant born to a mother exposed to study treatment.
Is a significant medical event in the investigator's judgment (e.g., may jeopardize the patient or may require medical/surgical intervention to prevent one of the outcomes listed above).

The terms “severe” and “serious” are not synonymous. Severity refers to the intensity of an adverse event (e.g., rated as mild, moderate, or severe, or according to NCI CTCAE; the event itself may be of relatively minor medical significance (such as severe headache without any further findings).

Adverse Events of Special Interest

Adverse events of special interest for the atezolizumab+tiragolumab arm are as follows:
Cases of potential drug-induced liver injury that include an elevated ALT or AST in combination with either elevated bilirubin or clinical jaundice, as defined by Hy's Law.
Suspected transmission of an infectious agent by the study treatment, as defined below:

Any organism, virus, or infectious particle (e.g., prion protein transmitting transmissible spongiform encephalopathy), pathogenic or non-pathogenic, is considered an infectious agent. A transmission of an infectious agent may be suspected from clinical symptoms or laboratory findings that indicate an infection in a patient exposed to a medicinal product. This term applies only when a contamination of study treatment is suspected.
Pneumonitis.
Colitis.
Endocrinopathies: diabetes mellitus, pancreatitis, adrenal insufficiency, and hypophysitis.
Hepatitis, including AST or ALT >10× upper limit of normal.
Systemic lupus erythematosus.
Neurological disorders: Guillain-Barré syndrome, myasthenic syndrome or myasthenia gravis, and meningoencephalitis.
Events suggestive of hypersensitivity, IRRs, cytokine-release syndrome, influenza-like illness, HLH, and MAS.
Nephritis.
Ocular toxicities (e.g., uveitis, retinitis, and optic neuritis).
Myositis.
Myopathies, including rhabdomyolysis.
Grade ≥2 cardiac disorders (e.g., atrial fibrillation, myocarditis, pericarditis).
Vasculitis.
Autoimmune hemolytic anemia.
Severe cutaneous reactions (e.g., Stevens-Johnson syndrome, dermatitis bullous, or toxic epidermal necrolysis).

### Example 19. Statistical Considerations and Analysis Plan for WO39613 Study

The WO39613 study analysis is based on patient data collected through study discontinuation. If not otherwise specified, efficacy analyses are based on the efficacy-evaluable population, defined as all patients who receive at least one dose of each drug for their assigned treatment regimen, and the safety analyses are based on the safety-evaluable population, defined as all patients who receive any amount of study treatment.

The analysis results are summarized by the treatment that patients actually receive. Data are described and summarized as warranted by sample size. Continuous variables are summarized through use of means, standard deviations, medians, and minimum and maximum values. Categorical variables are summarized through use of counts and percentages. Listings are used in lieu of tables in the event of small sample sizes.

A. Determination of Sample Size

The WO39613 study is not designed to make explicit power and type I error considerations for a hypothesis test. Instead, the study is designed to obtain preliminary efficacy, safety, and PK data on immunotherapy-based treatment combinations when administered to patients with cisplatin-ineligible MIBC. Approximately 150-350 patients are randomly allocated to the control and experimental arms during the study.

B. Efficacy Analyses

Primary Efficacy Endpoint

The primary efficacy endpoint is pathological complete response (pCR), defined as the proportion of patients with an absence of residual invasive cancer of the complete resected specimen. pCR rate is calculated for each arm, along with 90% confidence intervals. The difference in pCR between the experimental arm(s) and the control arm is also calculated, along with 90% confidence intervals. Confidence intervals are estimated by the exact method or the Wald method, depending on the sample size. Patients with missing or no response assessments are classified as non-responders.

Secondary Efficacy Endpoints

The secondary efficacy endpoints are landmark recurrence-free survival (RFS), landmark event-free survival (EFS), and landmark overall survival (OS), each at specific timepoints (e.g., 12, 18, 24 months). These endpoints are described below. Landmark RFS rates, landmark EFS rates, and landmark OS rates are estimated for each study arm using the Kaplan-Meier method, with 90% CIs calculated through use of Greenwood's formula.

Exploratory Efficacy Endpoints

The exploratory efficacy endpoints are RFS, EFS, OS, and pathological downstaging rate.

Recurrence-free survival (RFS) is defined as the time from Day 1 in the first cycle after surgery to the first documented recurrence of disease or death from any cause. For patients who do not have documented recurrence of disease or death, RFS is censored at the day of the last tumor assessment post surgery.

Event-free survival (EFS) is defined as the time from randomization to any of the following events (whichever occurs first): disease progression that precludes surgery, as assessed by the investigator; local or distant disease recurrence; or death from any cause. Patients who have not experienced such events are censored at the time of their last post-tumor tumor assessment.

Overall survival (OS) is defined as the time from randomization to death from any cause. Data for patients who have not died is censored at the last date known to be alive.

Pathological downstaging rate is defined as the proportion of patients that reach ≤pT1pN0 at the time of cystectomy.

The Kaplan-Meier method is used to estimate the median RFS, EFS, and OS for each study arm. The Brookmeyer and Crowley method are used to construct the 90% CI for the median RFS, EFS, and OS for each study arm.

C. Safety Analyses

Verbatim adverse event terms are mapped to Medical Dictionary for Regulatory Activities thesaurus terms, and adverse event severity is graded according to NCI CTCAE v4.0.

Safety is assessed through summaries of adverse events, changes in laboratory test results, changes in vital signs and ECGs, and exposure to study drugs. Exposure to combination treatment and length of safety follow-up is summarized by treatment arm within each stage.

Treatment-emergent adverse events occurring after initiation of treatment are summarized. For each patient, the maximum reported severity of each adverse event is used in the summaries by severity grade. All treatment-emergent adverse events, serious adverse events, adverse events leading to withdrawal of study treatment, Grade ≥3 adverse events, deaths, and causes of death are listed and summarized by mapped term, appropriate thesaurus level, and NCI CTCAE severity grade.

Relevant laboratory, vital sign (pulse rate, respiratory rate, blood pressure, pulse oximetry, and temperature), and ECG data are displayed by time, with grades identified where appropriate. Additionally, a shift table of selected laboratory test results is used to summarize the baseline and maximum postbaseline severity grade. Changes in vital signs are summarized.

D. Pharmacokinetic analyses

Sparse samples are collected for PK analyses of atezolizumab (patients who receive at least one dose of atezolizumab) and drugs given in combination with atezolizumab (patients who receive at least one dose of the drug). Serum or plasma concentrations of the various study drugs are reported as individual values and summarized (mean, standard deviation, coefficient of variation, median, range, geometric mean, and geometric mean coefficient of variation) by treatment arm and by cycle and day when appropriate and as data allow. Individual and median serum or plasma concentrations of the various study drugs are plotted by treatment arm and cycle and day. PK data for combination drugs may be compared with available historical data from internal and published previous studies. Concentration data may be pooled with data from other studies using an established population PK model to derive PK parameters such as clearance, volume of distribution, and area under the curve.

The relationship between PK parameters and safety, efficacy, PK, and biomarker endpoints may be analyzed and reported via descriptive statistics.

E. Immunogenicity Analyses

Immunogenicity is assessed for atezolizumab and other study treatments as appropriate. The immunogenicity analyses include all patients with at least one anti-drug antibody (ADA) assessment. Patients are grouped according to treatment received or, if no treatment is received prior to study discontinuation, according to treatment assigned.

For atezolizumab, the numbers and proportions of ADA-positive patients and ADA-negative patients at baseline (baseline prevalence) and after drug administration (postbaseline incidence) are summarized by treatment group. When determining postbaseline incidence, patients are considered to be ADA positive if they are ADA negative or have missing data at baseline but develop an ADA response following study drug exposure (treatment-induced ADA response), or if they are ADA positive at baseline and the titer of one or more postbaseline samples is at least 0.60-titer unit greater than the titer of the baseline sample (treatment-enhanced ADA response). Patients are considered to be ADA negative if they are ADA negative or have missing data at baseline and all postbaseline samples are negative, or if they are ADA positive at baseline but do not have any postbaseline samples with a titer that is at least 0.60-titer unit greater than the titer of the baseline sample (treatment unaffected).

For other study treatments where ADA is tested, positivity is determined according to standard methods established in previous studies of that drug.

The relationship between ADA status and safety, efficacy, PK, and biomarker endpoints may be analyzed and reported via descriptive statistics.

F. Biomarker Analyses

Exploratory biomarker analyses are performed in an effort to understand the association of these biomarkers with response to study drugs, taking into account efficacy, safety, PK, immunogenicity, and other biomarker endpoints.

G. Interim Analyses

Interim analyses are conducted during the study, with the earliest interim analysis taking place when at least one experimental arm has completed enrollment in the preliminary phase, and patients have completed their post-surgery pCR assessment. Further interim analyses may be conducted as deemed appropriate by Sponsor. A posterior probability may be used to guide further enrollment based on the interim analysis of clinical activity in the experimental arm compared with the control arm. If the interim analysis suggests that the activity in an experimental arm is higher than that in the control arm, there may be further enrollment of 25 additional patients in the experimental arm (expansion phase) for each MIBC Cohort. The final decision for expanding and ending the Phase 1b trial is made considering the overall benefit-risk balance and totality of data, including time-to-event endpoints and safety data, as well as emerging external information.

### Example 20. A Phase Ib/II, Open-Label, Multicenter, Randomized Umbrella Study Evaluating the Efficacy and Safety of Tiragolumab in Combination with Atezolizumab in Patients with Metastatic Urothelial Carcinoma (mUC)

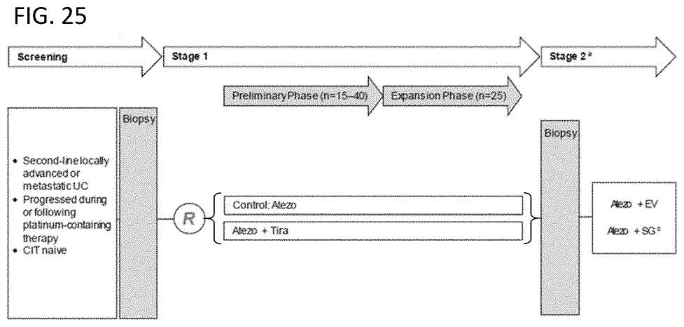

WO39613 is a Phase Ib/II, open-label, multicenter, randomized umbrella study evaluating the efficacy and safety of tiragolumab in combination with atezolizumab in patients with locally advanced or metastatic UC who have progressed during or following a platinum-containing regimen. The study is designed with the flexibility to open new treatment arms as new treatments become available, close existing treatment arms that demonstrate minimal clinical activity or unacceptable toxicity, or modify the patient population (e.g., with regard to prior anti-cancer treatment or biomarker status). Eligible patients are initially assigned to one of several treatment arms (see FIG. 25). Patients who experience loss of clinical benefit or unacceptable toxicity during Stage 1 may be eligible to continue treatment with a different treatment regimen for Stage 2 (see FIG. 25).

A. Stage 1

During Stage 1, patients are randomly assigned to a control arm (atezolizumab [Atezo]) or an experimental arm consisting of atezolizumab in combination with tiragolumab (Atezo+Tira) (see FIG. 25 and Table 76).

TABLE 76
Stage 1 treatment regimens

| | Number of patients | |
|---|---|---|
| Treatment^{a} | Preliminary phase | Expansion phase^{b} |
| Atezo (control) | Variable^{c} | Variable^{c} |
| Atezo + Tira | 15 | 25 |

Atezo = atezolizumab; Tira = tiragolumab.
^{a}The Sponsor may decide to delay or suspend enrollment within a given treatment arm. Thus, all experimental arms may not be open for enrollment at the same time.
^{b}lf clinical activity is observed in an experimental arm during the preliminary phase, approximately 25 additional patients are enrolled in that arm during the expansion phase.
^{c}The randomization ratio depends on the number of experimental arms that are open for randomization (e.g., if an arm is added or randomization into an arm is suspended pending analysis of results from the preliminary phase), with the stipulation that the likelihood of being allocated to the control arm is no more than 35%.

TABLE 77
Target and Proposed Mechanism-of-Action Classification
for Investigational Medicinal Products

| Experimental | | Proposed Mechanism-of- |
|---|---|---|
| IMP | Target(s) | Action Classification |
| Atezolizumab | PD-L1 | Immune checkpoint inhibitor. |
| Tiragolumab | TIGIT | TIGIT antagonist, |
| | | improves the activation |
| | | and effectiveness of T-cell and |
| | | NK-cell tumor-killing activity. |

PD-L1 = programmed death-ligand 1; TIGIT = T-cell immunoreceptor with Ig and ITIM domains.

Enrollment within the experimental arm takes place in two phases: a preliminary phase, followed by an expansion phase. Approximately 15 patients are enrolled during the preliminary phase. If clinical activity is observed in the experimental arm during the preliminary phase, approximately 25 additional patients may be enrolled in that arm during the expansion phase.

The Sponsor may decide to delay or suspend enrollment within a given treatment arm. Experimental arms with insufficient clinical activity or unacceptable toxicity are not expanded. Additional patients may be enrolled to ensure balance among treatment arms with respect to demographic and baseline characteristics, including potential predictive biomarkers, in order to enable further subgroup analyses. New experimental arms may be added during the study by amending the protocol.

Patients are randomly assigned to treatment arms, with the exception of the Stage 2 treatment arms. The randomization ratio also depends on the number of experimental arms that are available (e.g., if an arm is added or enrollment in an arm is suspended, pending analysis of results from the preliminary phase), with the stipulation that the likelihood of being allocated to the control arm is no more than 35%. Randomization takes into account arm-specific exclusion criteria. Patients are ineligible for a specific arm if they meet any of the exclusion criteria outlined for that arm (see below).

Patients in the atezolizumab control arm and the experimental arm are treated until unacceptable toxicity or loss of clinical benefit, as determined by the investigator after an integrated assessment of radiographic and biochemical data, local biopsy results (if available), and clinical status (e.g., symptomatic deterioration such as pain secondary to disease). Because of the possibility of an initial increase in tumor burden caused by immune-cell infiltration in the setting of a T-cell response (termed pseudoprogression) with cancer immunotherapies (CITs) (such as atezolizumab), radiographic progression per Response Evaluation Criteria in Solid Tumors, Version 1.1 (RECIST v1.1) may not be indicative of true disease progression. In the absence of unacceptable toxicity, patients who meet the criteria for disease progression per RECIST v1.1 while receiving treatment with a CIT drug are permitted to continue study treatment if they meet all of the following criteria:
Evidence of clinical benefit, as determined by the investigator following a review of all available data.
Absence of symptoms and signs (including laboratory values such as new or worsening hypercalcemia) indicating unequivocal progression of disease.
Absence of decline in ECOG PS that can be attributed to disease progression.
Absence of tumor progression at critical anatomical sites (e.g., leptomeningeal disease) that cannot be managed by protocol-allowed medical interventions.

B. Stage 2

During Stage 1, patients who experience loss of clinical benefit, as determined by the investigator, or unacceptable toxicity may be eligible to receive a different treatment combination during Stage 2 (See FIG. 25), provided they meet the eligibility criteria. Patients in Stage 2 who are eligible for more than one treatment arm are assigned a treatment arm by the investigator.

Stage 2 treatment must begin within 3 months after a patient has experienced loss of clinical benefit or unacceptable toxicity and continues until loss of clinical benefit or unacceptable toxicity as determined by the investigator. It is recommended that patients begin Stage 2 treatment as soon as possible.

The Sponsor may decide to hold or discontinue enrollment in Stage 2 treatment arms on the basis of a review of safety data, preliminary efficacy data, and supportive information (e.g., biomarker research data), as appropriate.

C. Rationale for Study Design

### Rationale for Patient Population and Atezolizumab Control Arm

The study enrolls patients with locally advanced or metastatic UC who have progressed during or following a platinum-containing regimen.

Atezolizumab (Tecentriq®) is approved for the treatment of UC in patients who have progressed during or following a platinum-containing regimen, non-small-cell lung cancer, small-cell lung cancer, triple-negative breast cancer, hepatocellular carcinoma, and melanoma. The approval for second-line treatment of UC patients who have progressed following a prior platinum-containing regimen was based on data from Cohort 2 of the ongoing Phase II study GO029293 (IMvigor210), which was followed by a full approval in the same indication in Europe based on results from both IMvigor210 and a Phase III study GO29294 (IMvigor211). In IMvigor210 Cohort 2, as of 12 Jul. 2017, 310 patients had been enrolled, and an ORR of 16% (95% CI: 13, 21) with a complete response (CR) rate of 7%, and a median duration of response (DOR) was 24.8 months (95% CI: 13.8, 30.4) was observed. Safety evaluation found that 8% of patients had an adverse event that led to treatment withdrawal, and 16% of patients had a treatment-related Grade 3 or 4 adverse event, with the most common events being fatigue, diarrhea, and pruritus. Immune-mediated events occurred in 12% of patients overall, with 7% of patients having a Grade 3 or 4 immune-mediated event (Grade 3 or 4 rash [1%], ALT increased [2%], blood bilirubin increased [2%], and rhabdomyolysis [1%]). In IMvigor211, as of 13 Mar. 2017, a median OS of 8.6 months, an ORR of 13.4% (10.5, 16.9), and a median DOR of 21.7 months was observed in the intent-to-treat (ITT) population.

Despite the promise of single-agent checkpoint inhibition, only a relatively small percentage of patients experience benefit, highlighting the need for immunotherapy combinations. The multiple combination partners in this study are expected to stimulate the immune system through a variety of mechanisms with the aim of extending the benefit of atezolizumab to a larger population of inoperable locally advanced and metastatic UC.

### Rationale for Stage 2

The advent of immunotherapy provided the first real advancement in mUC in 30 years. Despite this advancement, substantially more work needs to be done to extend the benefit to larger proportions of patients. Key to the ability to advance immunotherapy will be gaining a better understanding of the underlying tumor immune environment and how the sequencing of treatments will affect response.

The Stage 2 portion of this study allows some patients to proceed with subsequent CIT combinations after disease progression to advance the scientific understanding of immune-escape mechanisms in patients who fail to respond or progress on CIT regimens. Critical to patients' ability to enroll in Stage 2 is their ability to undergo a biopsy to allow for evaluation of the immune environment and any changes that may have happened during Stage 1 treatment. Participation in Stage 2 allows patients and providers access to treatment options with the potential for a durable response and improved toxicity profile not available through traditional chemotherapy. Stage 2 treatment would preclude treatment with other potentially more standard options; thus, patients will be informed during the Stage 2 consent process that by enrolling in Stage 2 they are foregoing therapy that may have a survival benefit.

### Rationale for Atezo+EV and Atezo+SG Arms in Stage 2

Patients may be given the opportunity to receive Stage 2 treatment with Atezo+EV or Atezo+SG after loss of clinical benefit.

The combination of Atezo+EV was chosen for Stage 2 because of the strong treatment responses documented in patients receiving single-agent enfortumab vedotin who had received prior CPI treatment. Specifically, in a Phase I trial of enfortumab vedotin, 8 of 17 (47%; 95% CI: 23, 72.2) patients with metastatic bladder treated at the recommended Phase II enfortumab vedotin dose obtained a PR (Petrylak et al., J Clin Oncol, 35(15 Suppl): 106, 2017). These findings suggest that enfortumab vedotin may provide benefit in the post-CPI space. The addition of atezolizumab to enfortumab vedotin has the potential to improve response durability. Additionally, microtubule-disrupting therapy has the potential to induce immunogenic cell death and initiate an anti-tumor immune response.

The combination of Atezo+SG was chosen for Stage 2 because of the strong treatment responses observed in patients with mUC receiving single-agent sacituzumab govitecan who had progressed on platinum-based therapy and checkpoint inhibitor therapy. Specifically, in the Phase II TROPHY-U-01 study, sacituzumab govitecan demonstrated an ORR of 29% in 35 patients including an ORR of 25.0% in patients with liver metastases (Tagawa et al., J Clin Oncol, 37(7 Suppl): 3199-3212, 2019). These findings suggest sacituzumab govitecan may provide benefit in the post-CPI space. The combination of atezolizumab with sacituzumab govitecan in patients who have progressed following chemo- and immunotherapy may provide synergy, by enabling atezolizumab to potentiate an antitumor immune response derived from the inflammation mediated by sacituzumab govitecan's anti-tumor activity.

### Rationale for Immunotherapy-Based Treatment Beyond Initial Radiographic Progression

In studies of immunotherapeutic agents, CR, partial response (PR), and stable disease have each been shown to occur after radiographic evidence of an apparent increase in tumor burden. This initial increase in tumor burden caused by immune-cell infiltration in the setting of a T-cell response has been termed pseudoprogression (Hales et al., Ann Oncol, 21: 1944-1951, 2010).

In Study PCD4989g, evidence of tumor growth followed by a response was observed in several tumor types. In addition, in some responding patients with radiographic evidence of progression, biopsies of new lesions or areas of new growth in existing lesions revealed immune cells and no viable cancer cells. Because of the potential for a response after pseudoprogression, the study allows patients randomly allocated to immunotherapy-based treatment arms to continue combination treatment after apparent radiographic progression per RECIST v1.1, provided the benefit-risk ratio is judged to be favorable by the investigator. Patients should be discontinued for unacceptable toxicity or loss of clinical benefit as determined by the investigator after an integrated assessment of radiographic and biochemical data, local biopsy results (if available), and clinical status.

Specific objectives and corresponding endpoints for the study are outlined in Tables 78 and 79.

TABLE 78
Objectives and corresponding endpoints for Stage 1

| Primary Efficacy Objective | Corresponding Endpoint |
|---|---|
| To evaluate the efficacy of immunotherapy- | Objective response rate, defined as the |
| based treatment combinations during Stage 1. | proportion of patients with a CR or PR |
| | on two consecutive occasions ≥ 4 weeks |
| | apart during Stage 1, as determined by |
| | the investigator according to RECIST v1.1. |
| Secondary Efficacy Objective | Corresponding Endpoints |
| To evaluate the efficacy of immunotherapy- | PFS after randomization, defined as the |
| based treatment combinations during Stage 1. | time from randomization to the first |
| | occurrence of disease progression or |
| | death from any cause (whichever occurs |
| | first) in Stage 1, as determined by the |
| | investigator according to RECIST v1.1. |
| | OS after randomization, defined as the |
| | time from randomization to death from |
| | any cause. |
| | OS rate at specific timepoints (e.g., 12 |
| | months), defined as the proportion of |
| | patients who have not experienced death |
| | from any cause at that timepoint. |
| | DOR, defined as the time from the first occurrence |
| | of a documented objective response during Stage |
| | 1 to disease progression or death from any cause |
| | (whichever occurs first), as determined by the |
| | investigator according to RECIST v1.1. |
| | Disease control, defined as stable disease ≥ 18 |
| | weeks or a CR or PR, as determined by the |
| | investigator according to RECIST v1.1. |
| Exploratory Efficacy Objective | Corresponding Endpoints |
| To evaluate the efficacy of immunotherapy- | Objective response, as determined by the |
| based treatment combinations during Stage 1. | investigator according to iRECIST. |
| | PFS after randomization, determined by the |
| | investigator according to iRECIST. |
| | DOR, as determined by the investigator |
| | according to iRECIST. |
| | Disease control, as determined by the |
| | investigator according to iRECIST. |
| Safety Objective | Corresponding Endpoint |
| To evaluate the safety of immunotherapy- | Incidence, nature, and severity of adverse events |
| based treatment combinations during Stage 1. | and laboratory abnormalities, with severity |
| | determined according to NCI CTCAE v4.0. |
| | Change from baseline in vital signs. |
| | Change from baseline in targeted clinical |
| | laboratory test results. |
| Exploratory Pharmacokinetic Objectives | Corresponding Endpoints |
| To characterize the PK profile of drugs that | Plasma or serum concentration of each drug (as |
| are administered as part of an immuno- | appropriate) at specified timepoints. |
| therapy-based treatment combination | |
| during Stage 1. | |
| To evaluate potential relationships between | Relationship between plasma or serum |
| drug exposure during Stage 1 and the | concentration or PK parameters for each drug (as |
| efficacy and safety of immunotherapy- | appropriate, on the basis of available data) and |
| based treatment combinations, | efficacy endpoints. |
| | Relationship between plasma or serum |
| | concentration or PK parameters for each drug (as |
| | appropriate, on the basis of available data) and |
| | safety endpoints. |
| Exploratory Immunogenicity Objectives | Corresponding Endpoint |
| To evaluate the immune response to drugs | For drugs for which ADA formation is measured: |
| that are administered as part of an immuno- | presence of ADAs during the study relative to the |
| therapy-based treatment combination during | presence of ADAs at baseline. |
| Stage 1. | For drugs for which ADA formation is measured: |
| To evaluate potential effects of ADAs | relationship between ADA status and efficacy, |
| during Stage 1. | safety, or PK endpoints. |
| Exploratory Biomarker Objective | Corresponding Endpoint |
| To identify biomarkers during Stage 1 that | Relationship between biomarkers in blood and |
| are predictive of response to study treat- | tumor tissue and efficacy, safety, PK, |
| ment (i.e., predictive biomarkers), are | immunogenicity, or other biomarker endpoints. |
| associated with progression to a more | |
| severe disease state (i.e., prognostic | |
| biomarkers), are associated with resistance | |
| to study treatment, are associated with | |
| susceptibility to developing adverse events | |
| (i.e., safety biomarkers), can provide | |
| evidence of study treatment activity (i.e., | |
| pharmacodynamic biomarkers), or can | |
| increase the knowledge and understanding | |
| of disease biology. | |

ADA = anti-drug antibody;
CR = complete response;
DOR = duration of response;
iRECIST = immune RECIST;
NCI CTCAE v4.0 = National Cancer Institute Common Terminology Criteria for Adverse Events, Version 4.0;
OS = overall survival;
PFS = progression-free survival;
PK = pharmacokinetic;
PR = partial response;
RECIST v1.1 = Response Evaluation Criteria in Solid Tumors, Version 1.1.

TABLE 79
Objectives and corresponding endpoints for Stage 2

| Exploratory Efficacy Objective | Corresponding Endpoints |
|---|---|
| To evaluate the efficacy of immunotherapy- | Objective response rate, defined as the proportion |
| based treatment combinations during | of patients with a CR or PR on two consecutive |
| Stage 2. | occasions ≥ 4 weeks apart during Stage 2, as |
| | determined by the investigator according to |
| | RECIST v1.1 and iRECIST. |
| | PFS after initiation of Stage 2, defined as the time |
| | from initiation of Stage 2 to the first occurrence of |
| | disease progression or death from any cause |
| | (whichever occurs first), as determined by the |
| | investigator according to RECIST v1.1 and |
| | iRECIST. |
| | DOR, defined as the time from the first occurrence |
| | of a documented objective response during Stage |
| | 2 to disease progression or death from any cause |
| | (whichever occurs first), as determined by the |
| | investigator according to RECIST v1.1 and |
| | iRECIST. |
| | Disease control, defined as stable disease for ≥ 18 |
| | weeks or a CR or PR, as determined by the |
| | investigator according to RECIST v1.1 and |
| | iRECIST. |
| Safety Objective | Corresponding Endpoint |
| To evaluate the safety of immunotherapy- | Incidence, nature, and severity of adverse events |
| based treatment combinations during | and laboratory abnormalities, with severity |
| Stage 2. | determined according to NCI CTCAE v4.0. |
| | Change from baseline in targeted vital signs. |
| | Change from baseline in targeted clinical |
| | laboratory test results. |
| Pharmacokinetic Objective | Corresponding Endpoint |
| To characterize the PK profile of drugs | Plasma or serum concentration of each drug (as |
| that are administered as part of an immuno- | appropriate) at specified timepoints. |
| therapy-based treatment combination | |
| during Stage 2. | |
| Immunogenicity Objective | Corresponding Endpoint |
| To evaluate the immune response to drugs | For drugs for which ADA formation is measured: |
| that are administered as part of an immuno- | presence of ADAs during the study relative to the |
| therapy- based treatment combination | presence of ADAs at baseline. |
| during Stage 2. | |
| Exploratory Immunogenicity Objective | Corresponding Endpoint |
| To evaluate potential effects of ADAs | For drugs for which ADA formation is measured: |
| during Stage 2. | relationship between ADA status and efficacy, |
| | safety, or PK, endpoints. |
| Exploratory Biomarker Objective | Corresponding Endpoint |
| To identify biomarkers during Stage 2 that | Relationship between biomarkers in blood and |
| are predictive of response to study treatment | tumor tissue and efficacy, safety, PK, |
| (i.e., predictive biomarkers), are associated | immunogenicity, or other biomarker endpoints. |
| with progression to a more severe disease | |
| state (i.e., prognostic biomarkers), are | |
| associated with resistance to study treatment, | |
| are associated with susceptibility to developing | |
| adverse events (i.e., safety biomarkers), can | |
| provide evidence of study treatment activity | |
| (i.e., pharmacodynamic biomarkers), or can | |
| increase the knowledge and understanding | |
| of disease biology. | |

ADA = anti-drug antibody;
CR = complete response;
DOR = duration of response;
iRECIST = immune RECIST;
NCI CTCAE v4.0 = National Cancer Institute Common Terminology Criteria for Adverse Events, Version 4.0;
OS = overall survival;
PFS = progression-free survival;
PK = pharmacokinetic;
PR = partial response;
RECIST v1.1 = Response Evaluation Criteria in Solid Tumors, Version 1.1.

D. Assessments and Monitoring

All patients are closely monitored for adverse events throughout the study, and adverse events are graded according to the National Cancer Institute Common Terminology Criteria for Adverse Events, Version 4.0 (NCI CTCAE v4.0). Patients undergo tumor assessments every 9 weeks (starting on Day 1 of Cycle 1) for the first 54 weeks and then every 12 weeks thereafter. Response is assessed by the investigator using RECIST v1.1 (Eisenhauer et al., Eur J Cancer, 45: 228-247, 2009)). If clinical activity is demonstrated in an experimental arm, the Sponsor may request that tumor assessment scans for that arm be submitted for evaluation by a central reading facility.

Baseline tumor tissue samples are collected from all patients, preferably by means of a biopsy performed at study entry. If a biopsy is not deemed feasible by the investigator, archival tumor tissue may be submitted. Tumor tissue is also collected from patients who discontinue Stage 1 because of unacceptable toxicity or loss of clinical benefit, as determined by the investigator (if deemed clinically feasible by the investigator). These samples, as well as blood samples collected during the study, are utilized for biomarker research.

To characterize the pharmacokinetic (PK) properties and/or immunogenicity of atezolizumab and other therapeutic agents, blood samples are obtained at various timepoints before and during study treatment administration.

E. End of Study and Length of Study

The end of the study is defined as the date when the last patient completes the last visit in both stages, including survival follow-up visits conducted by telephone or on-site visit.

The total length of the study, from screening of the first patient to the end of the study, will be approximately 3-5 years.

### Example 21. Materials and Methods for WO39613 mUC Study

A. Inclusion Criteria for Stage 1

Patients must meet all of the criteria outlined below to qualify for Stage 1.
Age ≥18 years at the time of signing Informed Consent Form.
Life expectancy ≥3 months, as determined by the investigator.
Histologically documented, locally advanced (T4b, any N; or any T, N2-N3) or metastatic UC (M1, Stage IV) (also termed TCC or urothelial cell carcinoma of the urinary tract; including renal pelvis, ureters, urinary bladder, and urethra). Patients with mixed histologies are required to have a dominant transitional cell pattern. Locally advanced bladder cancer must be inoperable on the basis of involvement of pelvic sidewall or adjacent viscera (clinical Stage T4b) or bulky nodal metastasis (N2-N3).
Availability of a representative tumor specimen that is suitable for determination of PD-L1 and/or additional biomarker status by means of central testing. Baseline tumor tissue samples are collected from all patients, preferably by means of a biopsy performed at study entry.
   A formalin-fixed, paraffin-embedded (FFPE) tumor specimen in a paraffin block (preferred) or at least 16 slides containing unstained, freshly cut, serial sections must be submitted along with an associated pathology report. If only 10-15 slides are available, the patient may still be eligible for the study. Tumor tissue should be of good quality based on total and viable tumor content.
   Fine-needle aspiration, brushing, cell pellet from pleural effusion, bone metastases, and lavage samples are not acceptable. For core-needle biopsy specimens, at least three cores should be submitted for evaluation. Patients with fewer than three cores available may still be eligible. TURBT specimens must contain a muscle invasive component (i.e., T2 or greater) of the bladder tumor as verified by local pathology review. If the TURBT specimens do not contain a muscle invasive component, then specimens obtained at the time of cystectomy/nephroureterectomy or metastatic spread (i.e., sample from a metastatic lesion) are required prior to randomization.
Disease progression during or following treatment with no more than one platinum-containing regimen (e.g., GC, MVAC, CarboGem) for inoperable, locally advanced or metastatic UC or disease recurrence. A regimen is defined as patients receiving at least two cycles of a platinum-containing regimen. Patients who received prior adjuvant/neoadjuvant chemotherapy and progressed within 12 months of treatment with a platinum-containing adjuvant/neoadjuvant regimen are considered as second-line patients. Patients may have received no more than two prior regimens of treatment (including the required platinum-based regimen) for their advanced or metastatic UC. Patients must have demonstrated disease progression during or following all prior regimen(s). Patients with disease progression following chemoradiotherapy must demonstrate progression outside the prior radiotherapy port.

B. Inclusion Criteria for Stage 1 and Stage 2

Patients must meet all of the criteria outlined below to qualify for Stage 1 or 2.
Ability to comply with the study protocol, in the investigator's judgment.
ECOG PS of 0 or 1.
Measurable disease (at least one target lesion) according to RECIST v1.1 Previously irradiated lesions can be considered as measurable disease only if progressive disease has been unequivocally documented at that site since radiation.
Adequate hematologic and end-organ function, defined by the following laboratory test results, obtained within 14 days prior to initiation of study treatment:
   ANC ≥1.5×10⁹/L (1500/μL) without granulocyte colony-stimulating factor (G-CSF) support.
   WBC count ≥2.5×10⁹/L (2500/μL).
   Lymphocyte count ≥0.5×10⁹/L (500/μL).
   Platelet count ≥100×10⁹/L (100,000/μL) without transfusion.
   Hemoglobin ≥90 g/L (9.0 g/dL).

Patients may be transfused; however, transfusions are not allowed within 2 weeks of screening laboratory tests for eligibility.

Patients with a solitary kidney or chronic kidney disease with low erythropoietin production may use erythropoietin-stimulating agents.
AST, ALT, and alkaline phosphatase (ALP) ≤2.5×upper limit of normal (ULN) with the following exceptions:

Patients with documented liver metastases: AST and/or ALT ≤5×ULN

Patients with documented liver or bone metastases: ALP ≤5×ULN.
Bilirubin ≤1.5×ULN, with the following exception:

Patients with known Gilbert disease: bilirubin level ≤3×ULN.
Albumin ≥25 g/L (2.5 g/dL).
Creatinine clearance ≥30 mL/min (calculated using the Cockcroft-Gault formula).
For patients not receiving therapeutic anticoagulation:
   PTT or aPTT ≤1.5×ULN
   PT or INR ≤1.5×ULN.
For patients receiving therapeutic anticoagulation: stable anticoagulant regimen during the 14 days prior to initiation of study treatment.
Negative HIV test at screening. Patients without a prior positive HIV test result will undergo an HIV test at screening, unless not permitted per local regulations.
Negative total hepatitis B core antibody (HBcAb) test at screening, or positive total HBcAb test followed by quantitative hepatitis B virus (HBV) DNA <500 IU/mL at screening. The HBV DNA test is performed only for patients who have a positive total HBcAb test.
Negative hepatitis C virus (HCV) antibody test at screening, or positive HCV antibody test followed by a negative HCV RNA test at screening. The HCV RNA test is performed only for patients who have a positive HCV antibody test.
For women of childbearing potential: agreement to remain abstinent (refrain from heterosexual intercourse) or use contraceptive measures and agreement to refrain from donating eggs.
For men: agreement to remain abstinent (refrain from heterosexual intercourse) or use contraceptive measures, and agreement to refrain from donating sperm.

C. Inclusion Criteria for Stage 2

Patients must meet all of the following criteria to qualify for Stage 2:
Patients in the atezolizumab control arm: ability to initiate Stage 2 treatment within 3 months after loss of clinical benefit as determined by the investigator while receiving control treatment.
Patients in an experimental arm during Stage 1: ability to initiate Stage 2 treatment within 3 months after experiencing unacceptable toxicity not related to atezolizumab or loss of clinical benefit as determined by the investigator while receiving Stage 1 treatment.
Availability of a tumor specimen from a biopsy performed upon discontinuation of Stage 1.

D. Exclusion Criteria

Patients who meet any of the following criteria are excluded from enrollment during Stage 1 and Stage 2. Event grades in the exclusion criteria are based on NCI CTCAE v4.0.

Exclusion Criteria for Stage 1

Patients who meet any of the following criteria are excluded from Stage 1:
Prior treatment with a T-cell co-stimulating therapy or a CPI including anti-CTLA-4, anti-PD-1, and anti-PD-L1 therapeutic antibodies.
Prior treatment with any of the protocol-specified study treatments including treatment with poly (adenosine diphosphate [ADP]-ribose) polymerase (PARP) inhibitor, nectin-4 targeting agents, signal regulatory protein α-targeting agents, TIGIT-targeting agents, Trop-2 targeting agents, FAP-directed therapies, 4-1 BB (CD137)-directed therapies, or topoisomerase 1 inhibitors.
Treatment with investigational therapy within 28 days prior to initiation of study treatment.
Any approved anti-cancer therapy, including chemotherapy or hormonal therapy, within 3 weeks prior to initiation of study treatment; the following exceptions are allowed:
   Palliative radiotherapy for bone metastases or soft tissue lesions should be completed >14 days prior to baseline imaging.
   Hormone-replacement therapy or oral contraceptives
Eligibility only for the control arm

Exclusion Criteria for Stage 1 and Stage 2

Patients who meet any of the following criteria are excluded from Stage 1 and from Stage 2:
Prior allogeneic stem cell or solid organ transplantation.
Treatment with systemic immunostimulatory agents (including, but not limited to, interferon and interleukin 2) within 4 weeks or 5 half-lives of the drug (whichever is longer) prior to the initiation of study treatment.
Treatment with systemic immunosuppressive medication (including, but not limited to, corticosteroids, cyclophosphamide, azathioprine, methotrexate, thalidomide, and anti-tumor necrosis factor-α agents) within 2 weeks prior to initiation of study treatment, or anticipation of need for systemic immunosuppressant medication during study treatment, with the following exceptions:

Patients who received acute, low-dose, systemic immunosuppressant medications, or a one-time pulse dose of systemic immunosuppressant medication (e.g., 48 hours of corticosteroids for a contrast allergy) are eligible for the study.

Patients who received mineralocorticoids (e.g., fludrocortisone), corticosteroids for chronic obstructive pulmonary disease or asthma, or low-dose corticosteroids for orthostatic hypotension or adrenal insufficiency are eligible for the study.
Treatment with a live, attenuated vaccine within 4 weeks prior to initiation of study treatment, or anticipation of need for such a vaccine during atezolizumab treatment or within 5 months after the last dose of atezolizumab.
Uncontrolled pleural effusion, pericardial effusion, or ascites requiring recurrent drainage procedures (once monthly or more frequently). Use of an indwelling catheter (e.g., PLEURX®) is allowed.
Uncontrolled tumor-related pain. Patients requiring pain medication must be on a stable regimen at study entry. Symptomatic lesions (e.g., bone metastases or metastases causing nerve impingement) amenable to palliative radiotherapy should be treated prior to enrollment. Patients should be recovered from the effects of radiation. There is no required minimum recovery period. Asymptomatic metastatic lesions that would likely cause functional deficits or intractable pain with further growth (e.g., epidural metastasis that is not presently associated with spinal cord compression) should be considered for loco-regional therapy if appropriate prior to enrollment.
Uncontrolled or symptomatic hypercalcemia (ionized calcium >1.5 mmol/L, calcium >12 mg/dL or corrected serum calcium >ULN).
Symptomatic, untreated, or actively progressing CNS metastases. Asymptomatic patients with treated CNS lesions are eligible, provided that all of the following criteria are met:
   Measurable disease, per RECIST v1.1, must be present outside the CNS.
   The patient has no history of intracranial hemorrhage or spinal cord hemorrhage.
   The patient has not undergone stereotactic radiotherapy within 7 days prior to initiation of study treatment, whole-brain radiotherapy within 14 days prior to initiation of study treatment, or neurosurgical resection within 28 days prior to initiation of study treatment.
   The patient has no ongoing requirement for corticosteroids as therapy for CNS disease. Anti-convulsant therapy at a stable dose is permitted.
History of leptomeningeal disease.
Active or history of autoimmune disease or immune deficiency, including, but not limited to, myasthenia gravis, myositis, autoimmune hepatitis, systemic lupus erythematosus, rheumatoid arthritis, inflammatory bowel disease, antiphospholipid antibody syndrome, Wegener granulomatosis, Sjögren syndrome, Guillain-Barré syndrome, or multiple sclerosis, with the following exceptions:

Patients with a history of autoimmune-related hypothyroidism who are on thyroid-replacement hormone are eligible for the study. Patients with controlled Type 1 diabetes mellitus who are on a stable insulin regimen are eligible for the study. Patients with eczema, psoriasis, lichen simplex chronicus, or vitiligo with dermatologic manifestations only (e.g., patients with psoriatic arthritis are excluded) are eligible for the study provided all of following conditions are met:
Rash must cover <10% of body surface area.
Disease is well controlled at baseline and requires only low-potency topical corticosteroids.
There is no occurrence of acute exacerbations of the underlying condition requiring psoralen plus ultraviolet A radiation, methotrexate, retinoids, biologic agents, oral calcineurin inhibitors, or high-potency or oral corticosteroids within the previous 12 months.
History of idiopathic pulmonary fibrosis, organizing pneumonia (e.g., bronchiolitis obliterans), drug-induced pneumonitis, or idiopathic pneumonitis, or evidence of active pneumonitis on screening chest computed tomography (CT) scan. History of radiation pneumonitis in the radiation field (fibrosis) is permitted.
History of malignancy other than UC within 2 years prior to screening, with the exception of malignancies with a negligible risk of metastasis or death (e.g., 5-year overall survival [OS] rate >90%), such as adequately treated carcinoma in situ of the cervix, non-melanoma skin carcinoma, localized prostate cancer, ductal carcinoma in situ, or Stage I uterine cancer. Patients with localized prostate cancer (defined as Stage ≤pT2c, Gleason score ≤7, and prostate-specific antigen (PSA) at prostate cancer diagnosis ≤20 ng/mL) treated with curative intent and without PSA recurrence are eligible. Patients with preexisting low-risk prostate cancer (defined as Stage cT1/T2a, Gleason score ≤6, and PSA ≤10 ng/mL) who are treatment-naive and undergoing active surveillance are eligible.
Active tuberculosis (TB) (i.e., has signs and symptoms of TB).
Severe infection within 4 weeks prior to initiation of study treatment, including, but not limited to, hospitalization for complications of infection, bacteremia, or severe pneumonia, or any active infection that, in the opinion of the investigator, could impact patient safety.
Treatment with therapeutic oral or IV antibiotics within 2 weeks prior to initiation of study Treatment. Patients receiving prophylactic antibiotics (e.g., to prevent a urinary tract infection or chronic obstructive pulmonary disease exacerbation) are eligible for the study.
Significant cardiovascular disease such as New York Heart Association cardiac disease (Class III or greater), myocardial infarction or cerebrovascular accident within 3 months prior to randomization, unstable arrhythmia, or unstable angina.
Uncontrolled hypertension (defined as systolic blood pressure >140 mmHg and/or diastolic blood pressure >95 mmHg). Anti-hypertensive therapy to achieve these parameters is allowed.
Grade ≥3 hemorrhage or bleeding event within 28 days prior to initiation of study treatment.
Major surgical procedure, other than for diagnosis, within 4 weeks prior to initiation of study treatment, or anticipation of need for a major surgical procedure during the study. Placement of central venous access catheter (e.g., port or similar) is not considered a major surgical procedure and is therefore permitted.
Adverse events from prior anti-cancer therapy that have not improved to Grade ≤1 or better, with the exception of alopecia of any grade and Grade ≤2 peripheral neuropathy.
Any other disease, metabolic dysfunction, physical examination finding, or clinical laboratory finding that contraindicates the use of an investigational drug, may affect the interpretation of the results, impair the ability of the patient to participate in the study, or may render the patient at high risk from treatment complications.
History of severe allergic reactions to chimeric or humanized antibodies or fusion proteins.
Known hypersensitivity to Chinese hamster ovary cell products or recombinant human antibodies.
Known intolerance or hypersensitivity to any of the study drugs of their excipients (including histidine, trehalose dihydrate, and polysorbate 20).
Known intolerance to any of the drugs required for premedication (acetaminophen, ranitidine, diphenhydramine, and methylprednisolone).
Patients entering Stage 2: inability to tolerate atezolizumab during Stage 1.
Pregnancy or breastfeeding, or intention of becoming pregnant during the study. Women of childbearing potential must have a negative serum pregnancy test result within 14 days prior to initiation of study treatment.
Patients entering Stage 2: immunotherapy-related adverse events that have not resolved to Grade 1 or better or to baseline at the time of consent with the following exception: patients with ongoing endocrine events that are adequately managed with supplemental therapy are eligible.

Additional Exclusion Criteria for Atezo+Tira Arm during Stage 1

Patients who meet any of the following criteria will be excluded from the Atezo+Tira arm during Stage 1:
Active EBV infection and known or suspected chronic active EBV infection at screening. Patients with a positive EBV viral capsid antigen IgM test at screening are excluded from this arm. An EBV polymerase chain reaction (PCR) test is performed as clinically indicated to screen for active infection or suspected chronic active infection. Patients with a positive EBV PCR test are excluded from this arm.

E. Permitted Therapy

Patients are permitted to use the following therapies during the study:
Oral contraceptives
Hormone-replacement therapy
Prophylactic or therapeutic anticoagulation therapy (such as warfarin at a stable dose or low-molecular-weight heparin at a stable dose)
Prophylactic antibiotic or anti-viral treatment administered according to institutional standards
Inactivated influenza vaccinations
Megestrol acetate administered as an appetite stimulant
Mineralocorticoids (e.g., fludrocortisone)
Corticosteroids administered for chronic obstructive pulmonary disease or asthma
Low-dose corticosteroids administered for orthostatic hypotension or adrenocortical insufficiency
Hormonal therapy with gonadotropin-releasing hormone agonists or antagonists for prostate cancer
Palliative radiotherapy (e.g., treatment of known bony metastases or symptomatic relief of pain) as outlined below:

After 2 cycles of treatment, palliative radiotherapy is permitted, provided it does not interfere with the assessment of tumor target lesions (e.g., the lesion to be irradiated must not be the only site of measurable disease). Treatment with atezolizumab may be continued during palliative radiotherapy.
Local therapy (e.g., surgery, stereotactic radiosurgery, radiotherapy, radiofrequency ablation) as outlined below:

Patients experiencing a mixed response requiring local therapy for control of three or fewer lesions may still be eligible to continue study treatment after Medical Monitor approval has been obtained. Patients who receive local therapy directed at a target lesion are no longer evaluable for radiographic response but will remain evaluable for progression.

Premedication with antihistamines, antipyretics, and/or analgesics may be administered for the second and subsequent atezolizumab infusions only, at the discretion of the investigator.

Patients who experience infusion-associated symptoms may be treated symptomatically with acetaminophen, ibuprofen, diphenhydramine, and/or H2-receptor antagonists (e.g., famotidine, cimetidine), or equivalent medications per local standard practice. Serious infusion-associated events manifested by dyspnea, hypotension, wheezing, bronchospasm, tachycardia, reduced oxygen saturation, or respiratory distress should be managed with supportive therapies as clinically indicated (e.g., supplemental oxygen and β2-adrenergic agonists).

D. Materials and Methods for Atezolizumab Arm

Atezolizumab is administered at a fixed dose of 1200 mg every 3 weeks (Q3W) (1200 mg on Day 1 of each 21-day cycle) (Table 80), which is the approved dosage (TECENTRIQ® U.S. Package Insert; TECENTRIQ® SmPC). Anti-tumor activity has been observed across doses ranging from 1 to 20 mg/kg Q3W. In Study PCD4989g, the maximum tolerated dose of atezolizumab was not reached, and no dose-limiting toxicities were observed at any dose. The fixed dose of 1200 mg Q3W (equivalent to an average body weight-based dose of 15 mg/kg Q3W) was selected on the basis of both nonclinical studies (Deng et al., MAbs, 8: 593-603, 2016) and available clinical pharmacokinetic, efficacy, and safety data.

Patients in the atezolizumab control arm receive treatment until unacceptable toxicity or loss of clinical benefit as determined by the investigator after an integrated assessment of radiographic and biochemical data, local biopsy results (if available), and clinical status (e.g., symptomatic deterioration such as pain secondary to disease). Treatment must be initiated no later than 7 days after treatment assignment. Patients undergo surgery after Cycle 3; treatment in Cycle 4 should be re-initiated 4-6 weeks after surgery.

TABLE 80
Treatment regimen for atezolizumab arm

| Cycle length | Dose, route, and regimen |
|---|---|
| 21 days | Atezolizumab 1200 mg IV on Day 1 of each cycle |

Administration of atezolizumab will be performed in a monitored setting where there is immediate access to trained personnel and adequate equipment and medicine to manage potentially serious reactions.

Atezolizumab infusions are administered per the instructions outlined in Table 81.

TABLE 81
Administration of first and subsequent atezolizumab infusions

| First infusion | Subsequent infusions |
|---|---|
| No premedication is permitted prior to the | If the patient experienced an infusion-related |
| atezolizumab infusion. | reaction with any previous infusion, premedication |
| | with antihistamines, antipyretics, and/or |
| | analgesics may be administered for subsequent |
| | doses at the discretion of the investigator. |
| Vital signs (pulse rate, respiratory rate, pulse | Vital signs should be recorded within 60 minutes |
| oximetry, blood pressure, and temperature) | prior to the infusion. |
| should be recorded within 60 minutes prior to the | |
| infusion. | |
| Atezolizumab should be infused over 60 (±15) | Atezolizumab should be infused over 30 (±10) |
| minutes. | minutes if the previous infusion was tolerated |
| If clinically indicated, vital signs should be | without an infusion-related reaction, or 60 (±15) |
| recorded every 15 (±5) minutes during the | minutes if the patient experienced an infusion- |
| infusion and 30 (±10) minutes after the infusion. | related reaction with the previous infusion. |
| Patients should be informed about the possibility | If the patient experienced an infusion-related |
| of delayed post-infusion symptoms and instructed | reaction with the previous infusion or if clinically |
| to contact their study physician if they develop | indicated, vital signs should be recorded during |
| such symptoms. | the infusion and at 30 (±10) minutes after the |
| | infusion. |

F. Materials and Methods for Atezolizumab+Tiragolumab Arm

Atezolizumab is administered as described above (see Table 81).

Tiragolumab is administered at a fixed dose of 600 mg IV Q3W (600 mg on Day 1 of each 21-day cycle). The fixed dose of 600 mg IV Q3W was selected on the basis of available clinical PK, efficacy, and safety data from the combined Phase Ia/Phase Ib Study GO30103, with single-agent tiragolumab or tiragolumab in combination with atezolizumab. In the Phase Ia portion of the study with tiragolumab as a single agent, the MTD was not reached, and no DLTs were observed in dose escalation. As of the clinical cutoff date, anti-drug antibodies (ADAs) to tiragolumab were rare in the Phase Ia or Phase Ib portions across all dose levels. Prolonged stable disease was observed in patients in the Phase Ia portion of the study at tiragolumab doses beginning at 400 mg. In the Phase Ib portion of the study with tiragolumab plus atezolizumab, the MTD was not reached. Anti-tumor activity, as measured by radiographic partial responses, was observed across doses for tiragolumab beginning at 30 mg and ranging up to 600 mg in combination with 1200 mg atezolizumab.

Patients in the atezolizumab+tiragolumab arm receive treatment as outlined in Table 82 until unacceptable toxicity or loss of clinical benefit as determined by the investigator after an integrated assessment of radiographic and biochemical data, local biopsy results (if available), and clinical status (e.g., symptomatic deterioration such as pain secondary to disease). Patients undergo surgery after Cycle 3; treatment in Cycle 4 is re-initiated 4-6 weeks after surgery.

TABLE 82
Treatment regimen for atezolizumab + tiragolumab arm

| | Dose, route, and regimen (drugs |
|---|---|
| Cycle length | listed in order of administration) |
| 21 days | Atezolizumab 1200 mg IV on Day 1 of each cycle |
| | Tiragolumab 600 mg IV on Day 1 of each cycle |

Tiragolumab is administered by IV infusion at a fixed dose of 600 mg on Day 1 of each 21-day cycle with a post-infusion observation period, as described in Table 83. Administration of tiragolumab is performed in a monitored setting where there is immediate access to trained personnel and adequate equipment and medicine to manage potentially serious reactions.

TABLE 83
Administration of first and subsequent tiragolumab infusions

| First infusion | Subsequent infusions |
|---|---|
| No premedication is permitted prior to the | If the patient experienced an infusion-related |
| tiragolumab infusion. | reaction with any previous infusion, premedication |
| | with antihistamines, antipyretics, and/or |
| | analgesics may be administered for subsequent |
| | doses at the discretion of the investigator. |
| Vital signs (pulse rate, respiratory rate, pulse | Vital signs should be recorded within 60 minutes |
| oximetry, blood pressure, and temperature) | prior to the infusion. |
| should be recorded within 60 minutes prior to the | |
| infusion. | |
| Tiragolumab should be infused over 60 (±10) | Tiragolumab should be infused over 30 (±10) |
| minutes. | minutes if the previous infusion was tolerated |
| | without an infusion-related reaction, or 60 (±10) |
| | minutes if the patient experienced an infusion- |
| | related reaction with the previous infusion. |
| After the infusion of tiragolumab, the patient | If the patient tolerated the previous infusion of |
| begins a 60-minute observation period. | tiragolumab well without infusion-associated |
| Record vital signs every 15 (±5) minutes during | adverse events, the observation period may be |
| the infusion and at 30 (±10) minutes after the | reduced to 30 minutes. |
| infusion. | |
| Patients are informed about the possibility of | If the patient experienced an infusion-related |
| delayed post-infusion symptoms and are | reaction with the previous infusion or if clinically |
| instructed to contact their study physician if they | indicated, vital signs should be recorded during |
| develop such symptoms. | the infusion and at 15 (±10) minutes after the |
| | infusion. |

Safety assessments for the WO39613 mUC study are performed as described in Example 18.

### Example 22. Statistical Considerations and Analysis Plan for WO39613 mUC Study

The WO39613 study analysis is based on patient data collected through study discontinuation. If not otherwise specified, efficacy analyses are based on the efficacy-evaluable population, defined as all patients who receive at least one dose of each drug for their assigned treatment regimen, and the safety analyses are based on the safety-evaluable population, defined as all patients who receive any amount of study treatment.

The analysis results are summarized by the treatment that patients actually receive, as well as by stage (Stage 1 or Stage 2), if applicable. Data are described and summarized as warranted by sample size. Continuous variables are summarized through use of means, standard deviations, medians, and minimum and maximum values. Categorical variables are summarized through use of counts and percentages. Listings are used in lieu of tables in the event of small sample sizes.

A. Determination of Sample Size

The WO39613 study is not designed to make explicit power and type I error considerations for a hypothesis test. Instead, the study is designed to obtain preliminary efficacy, safety, and PK data on immunotherapy-based treatment combinations when administered to patients with patients with locally advanced or metastatic UC who have progressed during or following a platinum-containing regimen. Approximately 160-385 patients are randomly allocated to the control and experimental arms during the study.

B. Efficacy Analyses

Primary Efficacy Endpoint

The primary efficacy endpoint is objective response rate (ORR) during Stage 1, as determined by the investigator according to RECIST v 1.1 and defined above (see Table 78). Patients with missing or no response assessments are classified as non-responders.

ORR, defined as the proportion of patients with a CR or PR, is calculated for each arm, along with 90% CIs (Clopper-Pearson exact method). The difference in ORR between the experimental arms and the control arm is also calculated, along with 90% CIs. CIs are estimated by the exact method or the Wald method, depending on the sample size.

Secondary Efficacy Endpoints

The secondary efficacy endpoints are progression-free survival (PFS), OS, OS at specific timepoints (e.g., 12 months), DOR, and disease control during Stage 1, as defined above (see Table 78). PFS, DOR, and disease control are determined by the investigator according to RECIST v1.1.

DOR is derived for efficacy-evaluable patients with a CR or PR.

For patients who do not have documented disease progression or death in a study stage, PFS and DOR are censored at the day of the last tumor assessment. Patients who are still alive at the time of OS analysis are censored at the last date they were known to be alive.

The Kaplan-Meier method is used to estimate the median for PFS, OS, and DOR, with 90% CIs constructed through use of the Brookmeyer and Crowley method. OS rate at specific timepoints is also estimated through use of the Kaplan-Meier method, with 90% CIs calculated on the basis of the Greenwood estimate for the variance.

Disease control rate, defined as the proportion of patients with stable disease for ≥18 weeks, a PR, or a CR, is calculated for each treatment arm, with 90% CIs estimated through use of the Clopper-Pearson exact method.

Exploratory Efficacy Endpoints

The exploratory efficacy endpoints are ORR, PFS, DOR, and disease control during Stage 1, as determined by the investigator according to iRECIST; and objective response, PFS, DOR, and disease control during Stage 2, as determined by the investigator according to RECIST v1.1 and iRECIST. ORR, PFS, DOR, and disease control are analyzed through use of the same methods described above. DOR is derived for efficacy-evaluable patients with a CR or PR.

C. Interim Analyses

Interim analyses are conducted during the study, with the earliest interim analysis taking place when at least one experimental arm has completed enrollment in the Stage 1 preliminary phase, and patients have been followed for a minimum of 9 weeks. Further interim analyses may be conducted as deemed appropriate. A posterior probability may be used to guide further enrollment based on the interim analysis of clinical activity in the experimental arm compared with the control arm. If the interim analysis suggests that the activity in an experimental arm is higher than that in the control arm, there may be further enrollment of 25 additional patients in the experimental arm (expansion phase). The final decision for expanding and ending this Phase 1 b trial will be made considering the overall benefit-risk balance and totality of data, including time-to-event endpoints and safety data, as well as emerging external information.

An interim analysis is also conducted after approximately 15 patients have been enrolled in a Stage 2 treatment arm and followed for a minimum of 9 weeks. If no clinical activity is observed in the Stage 2 treatment arm, further enrollment in that arm is stopped.

D. Other Analyses

Safety analyses, pharmacokinetic analyses, immunogenicity analyses, and biomarker analyses are performed as described in Example 19.

### Example 23. A Phase Ib/II, Open-Label, Multicenter, Randomized, Umbrella Study Evaluating the Efficacy and Safety of Immunotherapy-Based Treatment Combinations in Patients with Locally Advanced or Metastatic Esophageal Cancer

A. Overview of Study Design

YO039609 is a Phase Ib/II, open-label, multicenter, randomized, umbrella study evaluating the efficacy, safety, and pharmacokinetics of immunotherapy-based treatment combinations in patients with esophageal cancer. The study is designed to obtain preliminary efficacy, safety, and PK data on immunotherapy-based treatment combinations when administered to patients with esophageal cancer. The study is also designed with the flexibility to open new treatment arms as new treatments become available, close existing treatment arms that demonstrate minimal clinical activity or unacceptable toxicity, or modify the patient population (e.g., with regard to prior anti-cancer treatment or biomarker status).

Patients with esophageal cancer who have not received prior systemic treatment for their disease are enrolled in this study. Eligible patients are randomized to one of three treatment arms. Patients in the control arm who experience disease progression or unacceptable toxicity during Stage 1 may be eligible to continue treatment with a different treatment regimen in Stage 2.

Stage 1 Treatment

During Stage 1, patients with esophageal cancer are randomly assigned to a chemotherapy control arm (Cisplatin+5-EU) or one of two experimental arms: atezolizumab in combination with tiragolumab and chemotherapy (Atezo+Tiragolumab+Cisplatin+5-EU), or atezolizumab in combination with chemotherapy (Atezo+Cisplatin+5-EU). Details on the treatment regimens for Stage 1 are provided in Table 84.

TABLE 84
Stage 1 treatment regimens

| | | No. of Patients (Random Assignment)^{a} | |
|---|---|---|---|
| | Study Treatment | Preliminary Phase | Expansion Phase^{c} |
| 1L Esophageal | Atezo + Tira + Cisplatin + 5-FU | 40 | 25 |
| Cancer^{b} | Atezo + Cisplatin + 5-FU | 40 | 25 |

| | |
|---|---|
| Cisplatin + 5-FU | Variable |

1L = first-line;
5-FU = 5-fluorouracil;
Atezo = atezolizumab;
Tira = tiragolumab.
^{a}The randomization ratio depends on the number of experimental arms that are open for enrollment (e.g., if an arm is added or enrollment in an arm is suspended pending analysis of results from the preliminary phase), with the stipulation that no more than 35% of patients are randomly allocated to the control arm at a given time. The randomization ratios may be altered to increase enrollment in an experimental arm that has demonstrated promising clinical activity.
^{b}The Sponsor may decide to delay or suspend enrollment within a given treatment arm. Experimental arms with minimal clinical activity or unacceptable toxicity do not undergo expansion.
^{c}If clinical activity is observed in an experimental arm during the preliminary phase, approximately 25 additional patients are enrolled in that arm during the expansion phase.

Specific objectives and corresponding endpoints for Stage 1 of the study are outlined in Table 85.

TABLE 85
Objectives and Corresponding Endpoints for Stage 1

| | |
|---|---|
| Objectives | Corresponding Endpoint(s) |

Primary Efficacy Objective:

| | |
|---|---|
| To evaluate the efficacy of | Objective response, defined as a complete response |
| immunotherapy-based treatment | or a partial response on two consecutive occasions ≥4 |
| combinations in all arms during Stage 1 | weeks apart during Stage 1, as determined by the |
| | investigator according to RECIST v1.1 |

Secondary Efficacy Objectives:

| | |
|---|---|
| To evaluate the efficacy of | PFS after randomization, defined as the time from |
| immunotherapy-based treatment | randomization to the first occurrence of disease |
| combinations in all arms during Stage 1 | progression or death from any cause (whichever occurs |
| | first), as determined by the investigator according to |
| | RECIST v1.1 |
| | OS after randomization, defined as the time after |
| | randomization to death from any cause |
| | OS at specific timepoints (e.g., 6 and 12 months) |
| | DOR, defined as the time from the first occurrence of a |
| | documented objective response to disease progression |
| | or death from any cause (whichever occurs first) in |
| | Stage 1, as determined by the investigator according to |
| | RECIST v1.1 |
| | Disease control, defined as stable disease for ≥16 |
| | weeks or a complete or partial response, as determined |
| | by the investigator according to RECIST v1.1 |
| | Objective response in patients with TIGIT-positive |
| | tumors by IHC |
| | Objective response in patients with PD-L1 IC/TC- |
| | positive tumors by IHC |

Exploratory Efficacy Objectives:

| | |
|---|---|
| To evaluate the efficacy of | Objective response, as determined by the investigator |
| immunotherapy-based treatment | according to iRECIST |
| combinations in all experimental arms | PFS after randomization, as determined by the |
| during Stage 1 | investigator according to iRECIST. |
| | DOR, as determined by the investigator according to |
| | iRECIST. |
| | Disease control, as determined by the investigator |
| | according to iRECIST. |

Safety Objectives:

| | |
|---|---|
| To evaluate the safety of immunotherapy- | Incidence, nature, and severity of adverse events and |
| based treatment combinations in all arms | laboratory abnormalities, with severity determined |
| during Stage 1 | according to National Cancer Institute Common |
| | Terminology Criteria for Adverse Events, Version 4.0 |
| | Change from baseline in vital signs and ECG |
| | parameters |
| | Change from baseline in targeted clinical laboratory |
| | test results |

Pharmacokinetic Objective:

| | |
|---|---|
| To characterize the PK profile of drugs | Plasma or serum concentration of each drug (as |
| that are administered as part of an | appropriate) at specified timepoints |
| immunotherapy-based treatment | |
| combination during Stage 1 | |

Exploratory Pharmacokinetic Objective:

| | |
|---|---|
| To evaluate potential relationships | Relationship between plasma or serum concentration |
| between drug exposure during Stage 1 and | or PK parameters for each drug (as appropriate on the |
| the efficacy and safety of immunotherapy- | basis of available data) and efficacy endpoints |
| based treatment combinations | Relationship between plasma or serum concentration |
| | or PK parameters for each drug (as appropriate on the |
| | basis of available data) and safety endpoints |

Immunogenicity Objective:

| | |
|---|---|
| To evaluate the immune response to | For drugs for which ADA formation is measured, |
| drugs that are administered as part of an | presence of ADAs during the study relative to the |
| immunotherapy-based treatment | presence of ADAs at baseline |
| combination during Stage 1 | |

Exploratory Immunogenicity Objective:

| | |
|---|---|
| To evaluate the potential effects of ADAs | For drugs for which ADA formation is measured, |
| during Stage 1 | relationship between ADA status and efficacy, safety, or |
| | PK endpoints |

Exploratory Biomarker Objective:

| | |
|---|---|
| To identify biomarkers during Stage 1 that | Relationship between biomarkers in blood and tumor |
| are predictive of response to study | tissue and efficacy, safety, PK, immunogenicity, or other |
| treatment (i.e., predictive biomarkers), are | biomarker endpoints |
| associated with progression to a more | |
| severe disease state (i.e., prognostic | |
| biomarkers), are associated with resistance | |
| to study treatment, are associated with | |
| susceptibility to developing adverse events | |
| (i.e., safety biomarkers), can provide | |
| evidence of study treatment activity (i.e., | |
| pharmacodynamic biomarkers), or can | |
| increase the knowledge and understanding | |
| of disease biology | |

ADA = anti-drug antibody;
DOR = duration of response;
IC = immune cell;
IHC = immunohistochemistry;
iRECIST = modified RECIST v1.1 for immune-based therapeutics;
PFS = progression-free survival;
PK = pharmacokinetic;
RECIST v1.1 = Response Evaluation Criteria in Solid Tumors, Version 1.1;
TC = tumor cell;
TIGIT = T-cell immunoreceptor with Ig and ITIM domains.
Note:
Overall response at a single timepoint is assessed by the investigator using RECIST v1.1.

Enrollment for the two experimental arms takes place in two phases: a preliminary phase followed by an expansion phase.

Approximately 100-165 patients are enrolled during Stage 1. Up to approximately 40 patients are enrolled in the two experimental arms during the preliminary phase to ensure a sufficient number of patients with high TIGIT and PD-L1 expression to facilitate the evaluation of benefit and risk within this subpopulation. If clinical activity is observed in an experimental arm during the preliminary phase, approximately 25 additional patients may be enrolled in that arm during the expansion phase. The Sponsor may decide to delay or suspend enrollment within a given treatment arm.

Experimental arms with minimal clinical activity or unacceptable toxicity do not undergo expansion. New experimental arms may be added during the study by amending the protocol.

Patients in Stage 1 are randomly assigned to treatment arms, and the randomization ratio depends on the number of experimental arms that are available (e.g., if an arm is added or enrollment in an arm is suspended, pending analysis of results from the preliminary phase), with the stipulation that the likelihood of being allocated to the control arm is no more than 35%. The randomization ratio may be altered to increase enrollment in an experimental arm that has demonstrated promising clinical activity. Randomization takes into account arm-specific exclusion criteria. Patients are ineligible for a specific arm if they meet any of the exclusion criteria outlined for that arm.

Patients in the control arm are treated until unacceptable toxicity or disease progression per RECIST v1.1. Patients in the experimental arms are treated until unacceptable toxicity or loss of clinical benefit as determined by the investigator after an integrated assessment of radiographic and biochemical data, local biopsy results (if available), and clinical status (e.g., symptomatic deterioration such as pain secondary to disease). Because of the possibility of an initial increase in tumor burden caused by immune cell infiltration in the setting of a T-cell response (termed pseudoprogression) with cancer immunotherapies (CITs) (such as atezolizumab), radiographic progression per RECIST v1.1 may not be indicative of true disease progression. In the absence of unacceptable toxicity, patients who meet the criteria for disease progression per RECIST v1.1 while receiving treatment with a CIT drug are permitted to continue study treatment if they meet all of the following criteria:
Evidence of clinical benefit, as determined by the investigator following a review of all available data.
Absence of symptoms and signs (including laboratory values such as new or worsening hypercalcemia) indicating unequivocal progression of disease.
Absence of decline in ECOG Performance Status that can be attributed to disease progression.
Absence of tumor progression at critical anatomic sites (e.g., leptomeningeal disease) that cannot be managed by protocol-allowed medical interventions.
Patient's written consent to acknowledge deferring other treatment options in favor of continuing study treatment at the time of initial disease progression.

Stage 2 Treatment

Patients in the control arm who experience disease progression per RECIST v1.1 are given the option of receiving a different treatment combination during Stage 2 provided they meet eligibility criteria and the Stage 2 arm is open for enrollment. The Stage 2 treatment regimen is atezolizumab plus tiragolumab.

Patients in the control arm who experience unacceptable toxicity may be eligible for Stage 2 treatment after Medical Monitor approval has been obtained. Stage 2 treatment must begin within 3 months after the patient has experienced disease progression per RECIST v1.1 or unacceptable toxicity in Stage 1 and continues until unacceptable toxicity or loss of clinical benefit as determined by the investigator.

Specific objectives and corresponding endpoints for Stage 2 of the study are outlined in Table 86.

TABLE 86
Objectives and Corresponding Endpoints for Stage 2

| | |
|---|---|
| Objectives | Corresponding Endpoint(s) |

Exploratory Efficacy Objective:

| | |
|---|---|
| To evaluate the efficacy of | Objective response, defined as a complete response |
| immunotherapy-based treatment | or a partial response on two consecutive occasions ≥4 |
| combinations in patients during Stage 2 | weeks apart during Stage 2, as determined by the |
| | investigator according to RECIST v1.1 and iRECIST |
| | PFS after initiation of Stage 2, defined as the time |
| | from initiation of Stage 2 to the first occurrence of |
| | disease progression or death from any cause |
| | (whichever occurs first), as determined by the |
| | investigator according to RECIST v1.1 and iRECIST |
| | DOR, defined as the time from the first occurrence of a |
| | documented objective response during Stage 2 to |
| | disease progression or death from any cause |
| | (whichever occurs first) as determined by the |
| | investigator according to RECIST v1.1 and iRECIST |
| | Disease control, defined as stable disease for ≥16 |
| | weeks or a complete or partial response, as determined |
| | by the investigator according to RECIST v1.1 and |
| | iRECIST |

Safety Objective:

| | |
|---|---|
| To evaluate the safety of immunotherapy- | Incidence, nature, and severity of adverse events and |
| based treatment combinations during Stage | laboratory abnormalities, with severity determined |
| 2 | according to National Cancer Institute Common |
| | Terminology Criteria for Adverse Events, Version 4.0 |
| | Change from baseline in vital signs and ECG |
| | parameters |
| | Change from baseline in targeted clinical laboratory |
| | test results |

Pharmacokinetic Objective:

| | |
|---|---|
| To characterize the PK profile of drugs | Plasma or serum concentration of each drug at |
| that are administered as part of an | specified timepoints |
| immunotherapy-based treatment | |
| combination during Stage 2 | |

Exploratory Pharmacokinetic Objective:

| | |
|---|---|
| To evaluate potential relationships | Relationship between plasma or serum concentration |
| between drug exposure during Stage 2 and | or PK parameters for each drug (as appropriate on the |
| the efficacy and safety of immunotherapy- | basis of available data) and efficacy endpoints |
| based treatment combinations | Relationship between plasma or serum concentration |
| | or PK parameters for each drug (as appropriate on the |
| | basis of available data) and safety endpoints |

Immunogenicity Objective:

| | |
|---|---|
| To evaluate the immune response to | For drugs for which ADA formation is measured, |
| drugs that are administered as part of an | presence of ADAs during the study relative to the |
| immunotherapy-based treatment | presence of ADAs at baseline |
| combination during Stage 2 | |

Exploratory Immunogenicity Objective:

| | |
|---|---|
| To evaluate potential effects of ADAs | For drugs for which ADA formation is measured, |
| during Stage 2 | relationship between ADA status and efficacy, safety, or |
| | PK endpoints |

Exploratory Biomarker Objective:

| | |
|---|---|
| To identify biomarkers during Stage 2 that | Relationship between biomarkers in blood and tumor |
| are predictive of response to study | tissue and efficacy, safety, PK, immunogenicity, or other |
| treatment (i.e., predictive biomarkers), are | biomarker endpoints |
| associated with progression to a more | |
| severe disease state (i.e., prognostic | |
| biomarkers), are associated with resistance | |
| to study treatment, are associated with | |
| susceptibility to developing adverse events | |
| (i.e., safety biomarkers), can provide | |
| evidence of study treatment activity (i.e., | |
| pharmacodynamic biomarkers), or can | |
| increase the knowledge and understanding | |
| of disease biology | |

ADA = anti-drug antibody;
DOR = duration of response;
iRECIST = modified RECIST v1.1 for immune-based therapeutics;
OS = overall survival;
PFS = progression-free survival;
PK = pharmacokinetic;
RECIST v1.1 = Response Evaluation Criteria in Solid Tumors, Version 1.1.
Note:
Overall response at a single timepoint is assessed by the investigator using RECIST v1.1.

Stage 2 treatment must begin within 3 months after the patient has experienced disease progression per RECIST v1.1 or unacceptable toxicity in Stage 1 and continues until unacceptable toxicity or loss of clinical benefit as determined by the investigator. However, it is recommended that patients begin Stage 2 treatment as soon as possible.

The Sponsor may also decide to discontinue enrollment in the Stage 2 treatment arm on the basis of a review of safety data, preliminary efficacy data, and supportive information (e.g., biomarker research data), as appropriate.

B. End of Study and Length of Study

The end of the study is defined as the date when the last patient completes the last visit, including survival follow-up visits conducted by telephone or on-site visit.

The total length of the study, from screening of the first patient to the end of the study, will be approximately 3-6 years.

C. Rationale for Study Design and Patient Population

The study is designed to accelerate the development of cancer immunotherapy (CIT) combinations by identifying early signals and establishing proof-of-concept clinical data in patients with esophageal cancer.

CIT has demonstrated extraordinary success, with significant survival benefits observed across multiple advanced malignancies. Currently, the prevailing CIT approach is to circumvent immune evasion mechanisms and reinvigorate anti-tumor responses by identifying and targeting T-cell co-inhibitory surface receptors such as CTLA-4 and PD-L1/PD-1. Although these targets have resulted in remarkable clinical therapeutic success for various cancer indications, ongoing research indicates a series of stepwise events necessary for the generation of a continuous anti-tumor immune response (Chen and Mellman, Immunity, 39: 1-10, 2013). Each event is critical for an effective response, and each is also susceptible to several tumor immune evasion mechanisms. Thus, the need to identify and circumvent the various factors involved in tumor immune evasion is critical for propagating the anti-tumor immune response and advancing the field of CIT, most likely through combination regimens.

Esophageal cancer is a disease of high unmet medical need with 5-year survival rates of 5% in the metastatic and 24% in the locally advanced disease settings. Treatment options for patients with advanced or metastatic esophageal cancer are limited and have poor prognosis with minimal overall survival benefit. Therefore, there is a continuing need for more efficacious and better-tolerated treatments for this patient population.

### Example 24. Materials and Methods for YO39609 Study

Approximately 100-165 patients with esophageal cancer who have not received prior systemic therapy in this setting are randomized to the control and experimental arms during the YO39609 study.

K. Inclusion Criteria for Stage 1

Patients must meet all of the following criteria outlined below to qualify for Stage 1.
Age: ≥18 years at the time of signing Informed Consent Form.
ECOG Performance Status of 0 or 1.
Histologically or cytologically confirmed diagnosis of squamous cell carcinoma or adenocarcinoma of the esophagus in locally advanced or metastatic disease.
   The definitive diagnosis of metastatic esophageal cancer is made by evaluating the histopathologic data within the context of clinical and radiographic data.
No prior systemic treatment for esophageal cancer, with the following exception:
   For patients treated with chemotherapy in the locally advanced setting: occurrence of metastasis after 6 months from the last dose of chemotherapy.
For patients with adenocarcinoma: absence of HER2 expression as documented as ISH-negative on previously collected and assessed tumor tissue upon initial diagnosis of disease.
   HER2-negative status is determined on the basis of an ISH non-amplified (ratio of HER2 to CEP17 <2.0 or single probe average HER2 gene copy number <4 signals/cell) IHC 0 or IHC 1+ (if more than one test result is available and not all results meet the inclusion criterion definition, all results should be discussed with the Medical Monitor to establish eligibility of the patient).
Life expectancy: 3 months as determined by the investigator.
Creatinine clearance (CrCl): ≥50 mL/min (calculated using the Cockcroft-Gault formula).
Availability of a representative tumor specimen that is suitable for determination of PD-L1 and TIGIT levels by IHC and/or additional biomarker status by means of retrospective central testing.
   Baseline tumor tissue samples are collected from all patients, preferably by means of a biopsy performed at study entry. If a biopsy is not deemed feasible by the investigator, archival tumor tissue may be submitted after Medical Monitor approval, provided the tissue was obtained from a biopsy performed within 6 months prior to enrollment and the patient has not received any anti-cancer therapy since the time of the biopsy.
   A FFPE tumor specimen in a paraffin block (preferred) or at least 16 slides containing unstained, freshly cut, serial sections must be submitted along with an associated pathology report. If only 10-15 slides are available, the patient may still be eligible for the study, after Medical Monitor approval.

L. Inclusion Criteria for Stage 1 and Stage 2

Patients must meet all of the following criteria outlined below to qualify for Stages 1 and 2.
Signed Informed Consent Form.
Ability to comply with the study protocol, in the investigator's judgment.
Measurable disease per RECIST v1.1. Previously irradiated lesions can be considered as measurable disease only if progressive disease has been unequivocally documented at that site since radiation.
Adequate hematologic and end-organ function, defined by the following laboratory test results, obtained within 14 days prior to initiation of study treatment:
   ANC ≥1.5×10⁹/L (1500/μL) without granulocyte colony-stimulating factor support.
   Lymphocyte count 0.5×109/L (500/μL).
   Platelet count ≥100×10⁹/L (100,000/μL) without transfusion.
   Hemoglobin ≥90 g/L (9 g/dL). Patients may be transfused to meet this criterion.
   AST, ALT, and alkaline phosphatase (ALP)≤2.5× upper limit of normal (ULN), with the following exceptions:
      Patients with documented liver metastases: AST and ALT ≤5×ULN.
      Patients with documented liver or bone metastases: ALP ≤5×ULN.
   Bilirubin ≤1.5×ULN with the following exception:
      Patients with known Gilbert disease: bilirubin level ≤3×ULN.
   Albumin 25 g/L (2.5 g/dL).
   For patients not receiving therapeutic anticoagulation: INR or aPTT ≤1.5×ULN.
   For patients receiving therapeutic anticoagulation: stable anticoagulant regimen.
Negative HIV test at screening, with the following exception: patients with a positive HIV test at screening are eligible provided they are stable on anti-retroviral therapy, have a CD4 count 200/μL, and have an undetectable viral load.
Negative hepatitis B surface antigen (HBsAg) test at screening.
Negative total hepatitis B core antibody (HBcAb) test at screening, or positive total HBcAb test followed by quantitative hepatitis B virus (HBV) DNA <500 IU/mL at screening. The HBV DNA test is performed only for patients who have a positive total HBcAb test.
Negative hepatitis C virus (HCV) antibody test at screening, or positive HCV antibody test followed by a negative HCV RNA test at screening. The HCV RNA test is performed only for patients who have a positive HCV antibody test.
For women of childbearing potential: agreement to remain abstinent (refrain from heterosexual intercourse) or use contraceptive measures, and agreement to refrain from donating eggs.
For men: agreement to remain abstinent (refrain from heterosexual intercourse) or use contraceptive measures, and agreement to refrain from donating sperm.

M. Inclusion Criteria for Stage 2

Patients must meet all of the following criteria outlined below to qualify for Stage 2.
ECOG Performance Status of 0, 1, or 2.
Patients randomly allocated to the control arm during Stage 1: ability to initiate Stage 2 treatment within 3 months after experiencing unacceptable toxicity, provided that Medical Monitor approval for entry into Stage 2 is obtained, or disease progression per RECIST v1.1 while receiving control treatment.
Creatinine ≤1.5×ULN or CrCl 30 mL/min (calculated using the Cockcroft-Gault formula).
Availability of a tumor specimen (if deemed clinically feasible) from a biopsy performed upon discontinuation of Stage 1 because of unacceptable toxicity, or disease progression per RECIST v1.1.

N. Exclusion Criteria

Patients who meet any of the following criteria are excluded from enrollment during Stage 1 and Stage 2.

Exclusion Criteria for Stage 1

Patients who meet any of the following criteria are excluded from Stage 1:
Prior treatment with any of the protocol-specified study treatments.
Patients with known hearing impairment.
Grade ≥2 peripheral neuropathy as defined by NCI CTCAE v4.0 criteria.
Known dihydropyrimidine dehydrogenase deficiency or thymidylate synthase gene polymorphism predisposing the patient to 5-FU toxicity.

Exclusion Criteria for Stage 1 and Stage 2

Patients who meet any of the following criteria are excluded from Stage 1 and from Stage 2:
High risk for developing esophageal fistula by clinical assessment or imaging, such as prior history or associated symptoms of esophageal fistula or T4 classification by endoscopic ultrasound.
Symptomatic, untreated, or actively progressing central nervous system (CNS) metastases. Asymptomatic patients with treated CNS lesions are eligible, provided that all of the following criteria are met:
   Measurable disease, per RECIST v1.1, must be present outside the CNS.
   The patient has no history of intracranial hemorrhage or spinal cord hemorrhage.
   The patient has not undergone stereotactic radiotherapy within 7 days prior to initiation of study treatment, whole-brain radiotherapy within 14 days prior to initiation of study treatment, or neurosurgical resection within 28 days prior to initiation of study treatment.
   The patient has no ongoing requirement for corticosteroids as therapy for CNS disease. Anticonvulsant therapy at a stable dose is permitted.
   Metastases are limited to the cerebellum or the supratentorial region (i.e., no metastases to the midbrain, pons, medulla, or spinal cord).
   There is no evidence of interim progression between completion of CNS-directed therapy and initiation of study treatment.
   Asymptomatic patients with CNS metastases newly detected at screening are eligible for the study after receiving radiotherapy or surgery, with no need to repeat the screening brain scan.
Active EBV infection and known or suspected chronic active EBV infection at screening. Patients who are positive for EBV IgG and/or Epstein-Barr nuclear antigen (EBNA) are eligible only if EBV IgM and/or EBV PCR are negative.
History of leptomeningeal disease.
Uncontrolled tumor-related pain. Patients requiring pain medication must be on a stable regimen at study entry. Symptomatic lesions (e.g., bone metastases or metastases causing nerve impingement) amenable to palliative radiotherapy should be treated prior to enrollment. Patients should be recovered from the effects of radiation. There is no required minimum recovery period. Asymptomatic metastatic lesions that would likely cause functional deficits or intractable pain with further growth (e.g., epidural metastasis that is not currently associated with spinal cord compression) should be considered for loco-regional therapy if appropriate prior to enrollment.
Uncontrolled pleural effusion, pericardial effusion, or ascites requiring recurrent drainage procedures (once monthly or more frequently). Patients with indwelling catheters (e.g., PLEURX®) are allowed.
Uncontrolled or symptomatic hypercalcemia (ionized calcium >1.5 mmol/L, calcium >12 mg/dL, or corrected calcium >ULN).
Active or history of autoimmune disease or immune deficiency, including, but not limited to, myasthenia gravis, myositis, autoimmune hepatitis, systemic lupus erythematosus, rheumatoid arthritis, inflammatory bowel disease, antiphospholipid antibody syndrome, Wegener granulomatosis, Sjögren syndrome, Guillain-Barr6 syndrome, or multiple sclerosis, with the following exceptions:

Patients with a history of autoimmune-related hypothyroidism who are on thyroid-replacement hormone are eligible for the study.

Patients with controlled Type 1 diabetes mellitus who are on an insulin regimen are eligible for the study.

Patients with eczema, psoriasis, lichen simplex chronicus, or vitiligo with dermatologic manifestations only (e.g., patients with psoriatic arthritis are excluded) are eligible for the study provided all of following conditions are met:
Rash must cover <10% of body surface area.
Disease is well controlled at baseline and requires only low-potency topical corticosteroids.
No occurrence of acute exacerbations of the underlying condition requiring psoralen plus ultraviolet A radiation, methotrexate, retinoids, biologic agents, oral calcineurin inhibitors, or high-potency or oral corticosteroids within the previous 12 months.
History of idiopathic pulmonary fibrosis, organizing pneumonia (e.g., bronchiolitis obliterans), drug-induced pneumonitis, or idiopathic pneumonitis, or evidence of active pneumonitis on screening chest computed tomography (CT) scan. History of radiation pneumonitis in the radiation field (fibrosis) is permitted.
Active tuberculosis.
Significant cardiovascular disease (such as New York Heart Association Class II or greater cardiac disease, myocardial infarction, or cerebrovascular accident) within 3 months prior to initiation of study treatment, unstable arrhythmia, or unstable angina.
Major surgical procedure, other than for diagnosis, within 4 weeks prior to initiation of study treatment, or anticipation of need for a major surgical procedure during the study.
History of malignancy other than esophageal cancer within 2 years prior to screening, with the exception of malignancies with a negligible risk of metastasis or death (e.g., 5-year OS rate >90%), such as adequately treated carcinoma in situ of the cervix, non-melanoma skin carcinoma, localized prostate cancer, ductal carcinoma in situ, or Stage I uterine cancer.
Severe infection within 4 weeks prior to initiation of study treatment, including, but not limited to, hospitalization for complications of infection, bacteremia, or severe pneumonia.
Treatment with therapeutic oral or IV antibiotics within 2 weeks prior to initiation of study treatment.
Patients receiving prophylactic antibiotics (e.g., to prevent a urinary tract infection or chronic obstructive pulmonary disease exacerbation) are eligible for the study.
Prior allogeneic stem cell or solid organ transplantation.
Any other disease, metabolic dysfunction, physical examination finding, or clinical laboratory finding that contraindicates the use of an investigational drug, may affect the interpretation of the results, or may render the patient at high risk from treatment complications.
Treatment with a live, attenuated vaccine within 4 weeks prior to initiation of study treatment, or anticipation of need for such a vaccine during atezolizumab treatment or within 5 months after the final dose of atezolizumab.
Current treatment with anti-viral therapy for HBV.
Known allergy or hypersensitivity to any of the study drugs or their excipients.
Treatment with investigational therapy within 28 days prior to initiation of study treatment.
Prior treatment with CD137 agonists or immune checkpoint blockade therapies, including anti-CTLA-4, anti-PD-1, anti-PD-L1, and anti-TIGIT therapeutic antibodies.
Treatment with systemic immunostimulatory agents (including, but not limited to, interferon and interleukin 2 [IL-2]) within 4 weeks or 5 drug elimination half-lives (whichever is longer) prior to initiation of study treatment.
Treatment with systemic immunosuppressive medication (including, but not limited to, corticosteroids, cyclophosphamide, azathioprine, methotrexate, thalidomide, and anti-TNF-α agents) within 2 weeks prior to initiation of study treatment, or anticipation of need for systemic immunosuppressive medication during study treatment, with the following exceptions:
   Patients who received acute, low-dose systemic immunosuppressant medication or a one-time pulse dose of systemic immunosuppressant medication (e.g., 48 hours of corticosteroids for a contrast allergy) are eligible for the study after Medical Monitor approval has been obtained.
   Patients who received mineralocorticoids (e.g., fludrocortisone), corticosteroids for chronic obstructive pulmonary disease (COPD) or asthma, or low-dose corticosteroids for orthostatic hypotension or adrenal insufficiency are eligible for the study.
History of severe allergic anaphylactic reactions to chimeric or humanized antibodies or fusion proteins.
Known hypersensitivity to Chinese hamster ovary cell products or to any component of the atezolizumab and tiragolumab formulation.
Pregnancy or breastfeeding, or intention of becoming pregnant during study treatment or within 5 months after the final dose of atezolizumab and 5 months after the final dose of tiragolumab. Women of childbearing potential must have a negative serum pregnancy test result within 14 days prior to initiation of study treatment.

O. Permitted Therapy

Patients are permitted to use the following therapies during the study:
Oral contraceptives
Hormone-replacement therapy
Prophylactic or therapeutic anticoagulation therapy (such as warfarin at a stable dose or low-molecular-weight heparin)
Inactivated influenza vaccinations
Megestrol acetate administered as an appetite stimulant
Mineralocorticoids (e.g., fludrocortisone)
Corticosteroids administered for chronic obstructive pulmonary disease or asthma
Low-dose corticosteroids administered for orthostatic hypotension or adrenocortical insufficiency
Palliative radiotherapy (e.g., treatment of known bony metastases or symptomatic relief of pain) as outlined below:

Palliative radiotherapy is permitted, provided it does not interfere with the assessment of tumor target lesions (e.g., the lesion to be irradiated must not be the only site of measurable disease). Treatment with atezolizumab may be continued during palliative radiotherapy.
Local therapy (e.g., surgery, stereotactic radiosurgery, radiotherapy, radiofrequency ablation) as outlined below:

Patients experiencing a mixed response requiring local therapy for control of three or fewer lesions may still be eligible to continue study treatment after Medical Monitor approval has been obtained. Patients who receive local therapy directed at a target lesion are no longer evaluable for radiographic response but remain evaluable for progression.

Premedication with antihistamines, antipyretics, and/or analgesics may be administered for the second and subsequent atezolizumab infusions only, at the discretion of the investigator.

Patients who experience infusion-associated symptoms may be treated symptomatically with acetaminophen, ibuprofen, diphenhydramine, and/or H2-receptor antagonists (e.g., famotidine, cimetidine), or equivalent medications per local standard practice. Serious infusion-associated events manifested by dyspnea, hypotension, wheezing, bronchospasm, tachycardia, reduced oxygen saturation, or respiratory distress should be managed with supportive therapies as clinically indicated (e.g., supplemental oxygen and β₂-adrenergic agonists).

P. Dosing and Administration

Patients in the treatment arms below receive treatment as outlined until unacceptable toxicity or loss of clinical benefit as determined by the investigator after an integrated assessment of radiographic and biochemical data, local biopsy results (if available), and clinical status (e.g., symptomatic deterioration such as pain secondary to disease).

Atezolizumab+tiragolumab+cisplatin+5-fluouracil arm

Patients in the atezolizumab+tiragolumab+cisplatin+5-FU arm receive treatment as outlined in Table 87.

TABLE 87
Treatment regimen for atezolizumab + tiragolumab + cisplatin +
5-FU arm

| Cycle | Dose, Route, and Regimen |
|---|---|
| Length | (Drugs Listed in Order of Administration) |
| 21 days | Atezolizumab 1200 mg IV infusion on Day 1 of each cycle |
| | Tiragolumab 600 mg IV infusion on Day 1 of each cycle |
| | Cisplatin 80 mg/m² IV infusion on Day 1 of each cycle^{a,b} |
| | 5-FU 800 mg/m²/24 hours IV infusion (continuous) on |
| | Days 1-5 of each cycle |

5-FU = 5-fluorouracil;
^{a}Cisplatin treatment is capped after 6 doses.
^{b}Infusion times for cisplatin and 5-FU may be adapted in accordance with local standard practice.

Atezolizumab+Cisplatin+5-Fluouracil Arm

Patients in the atezolizumab+cisplatin+5-FU arm receive treatment as outlined in Table 88.

TABLE 88
Treatment regimen for atezolizumab + cisplatin + 5-FU arm

| Cycle | Dose, Route, and Regimen |
|---|---|
| Length | (Drugs Listed in Order of Administration) |
| 21 days | Atezolizumab 1200 mg IV infusion on Day 1 of each cycle |
| | Cisplatin 80 mg/m² IV infusion on Day 1 of each cycle^{a,b} |
| | 5-FU 800 mg/m²/24 hours IV infusion (continuous) on |
| | Days 1-5 of each cycle |

5-FU = 5-fluorouracil;
^{a}Cisplatin treatment is capped after 6 doses.
^{b}Infusion times for cisplatin and 5-FU may be adapted in accordance with local standard practice.

Control arm (cisplatin+5-FU)

Cisplatin is administered by IV infusion over 120 minutes at a dose of 80 mg/m² on Day 1 of the first six 21-day cycles. 5-FU is administered by continuous IV infusion over Days 1-5 (120 hours) at a dose of 800 mg/m²/24 hours starting on Day 1 of each 21-day cycle. Cisplatin treatment continues for a total of 6 cycles, unless unacceptable toxicity occurs, or the patient is withdrawn from study treatment for any reason. Patients in the Cisplatin+5-FU control arm receive treatment until unacceptable toxicity or disease progression per RECIST v1.1.

Stage 2 Treatment for the Control Arm (Atezolizumab+Tiragolumab)

Patients in the atezolizumab+tiragolumab arm receive treatment as outlined in Table 89.

TABLE 89
Treatment regimen for atezolizumab + tiragolumab arm

| Cycle | Dose, Route, and Regimen |
|---|---|
| Length | (Drugs Listed in Order of Administration) |
| 21 days | Atezolizumab 1200 mg IV infusion on Day 1 of each cycle |
| | Tiragolumab 600 mg IV infusion on Day 1 of each cycle |

Patients who experience disease progression per RECIST v1.1 are given the option of receiving a different treatment combination during Stage 2 of the study provided they meet eligibility criteria and the Stage 2 arm is open for enrollment.

Patients who experience unacceptable toxicity may also be eligible to receive treatment during Stage 2, provided they meet eligibility criteria and after Medical Monitor approval has been obtained. Stage 2 treatment must begin within 3 months after the patient has experienced disease progression or unacceptable toxicity. Tumor assessments performed prior to or at the time of disease progression or unacceptable toxicity during Stage 1 may serve as baseline assessments for Stage 2, provided the tumor assessments are performed within 28 days prior to initiation of Stage 2 treatment (i.e., Day 1 of Cycle 1). Stage 2 treatment continues until unacceptable toxicity or loss of clinical benefit as determined by the investigator.

Atezolizumab infusions are administered per the instructions outlined in Table 90.

TABLE 90
Administration of first and subsequent atezolizumab infusions

| First infusion | Subsequent infusions |
|---|---|
| No premedication is permitted prior to the | If the patient experienced an infusion-related |
| atezolizumab infusion. | reaction with any previous infusion, premedication |
| | with antihistamines, antipyretics, and/or |
| | analgesics may be administered for subsequent |
| | doses at the discretion of the investigator. |
| Vital signs (pulse rate, respiratory rate, pulse | Vital signs should be recorded within 60 minutes |
| oximetry, blood pressure, and temperature) | prior to the infusion. |
| should be recorded within 60 minutes prior to the | |
| infusion. | |
| Atezolizumab should be infused over 60 (±15) | Atezolizumab should be infused over 30 (±10) |
| minutes. | minutes if the previous infusion was tolerated |
| | without an infusion-related reaction, or 60 (±15) |
| | minutes if the patient experienced an infusion- |
| | related reaction with the previous infusion. |
| After the infusion of aterzolizumab, the patient | If the patient experienced an infusion-related |
| begins a 30-minute observation period | reaction with the previous infusion or if clinically |
| If clinically indicated, vital signs should be | indicated, vital signs should be recorded during |
| recorded every 15 (±5) minutes during the | the infusion and at 30 (±10) minutes after the |
| infusion and 30 (±10) minutes after the infusion. | infusion. |
| Patients should be informed about the possibility | |
| of delayed post-infusion symptoms and instructed | |
| to contact their study physician if they develop | |
| such symptoms. | |

Tiragolumab infusions are administered per the instructions outlined in Table 91.

TABLE 91
Administration of first and subsequent tiragolumab infusions

| First infusion | Subsequent infusions |
|---|---|
| No premedication is permitted prior to the | If the patient experienced an infusion-related |
| tiragolumab infusion, | reaction with any previous infusion, premedication |
| | with antihistamines, antipyretics, and/or |
| | analgesics may be administered for subsequent |
| | doses at the discretion of the investigator. |
| Vital signs (pulse rate, respiratory rate, pulse | Vital signs should be recorded within 60 minutes |
| oximetry, blood pressure, and temperature) | prior to the infusion. |
| should be recorded within 60 minutes prior to the | |
| infusion. | |
| Tiragolumab should be infused over 60 (±10) | Tiragolumab should be infused over 30 (±10) |
| minutes. | minutes if the previous infusion was tolerated |
| | without an infusion-related reaction, or 60 (±10) |
| | minutes if the patient experienced an infusion- |
| | related reaction with the previous infusion. |
| After the infusion of tiragolumab, the patient | If the patient tolerated the previous infusion of |
| begins a 60-minute observation period. | tiragolumab well without infusion-associated |
| Record vital signs every 15 (±5) minutes during | adverse events, the observation period may be |
| the infusion and at 30 (±10) minutes after the | reduced to 30 minutes. |
| infusion. | If the patient experienced an infusion-related |
| Patients are informed about the possibility of | reaction with the previous infusion or if clinically |
| delayed post-infusion symptoms and are | indicated, vital signs should be recorded during |
| instructed to contact their study physician if they | the infusion and at 15 (±10) minutes after the |
| develop such symptoms. | infusion. |

Q. Safety evaluation phase

To account for potential overlapping toxicities in the atezolizumab+tiragolumab+cisplatin+5-FU arm, enrollment is suspended after approximately 6 patients have been enrolled to allow for a safety evaluation. The safety evaluation is based on safety data from a minimum of 6 patients who have received at least one dose of treatment (i.e., one dose of each agent for a given combination) and completed safety follow-up during at least one full treatment cycle. If the combination is determined to be sufficiently safe, enrollment resumes in the arm.

R. Assessments and Monitoring

All patients are closely monitored for adverse events throughout the study, and adverse events are graded according to the National Cancer Institute Common Terminology Criteria for Adverse Events, Version 4.0 (NCI CTCAE v4.0). Patients undergo tumor assessments every 6 weeks (starting on Day 1 of Cycle 1) for the first 12 months and then every 6 or 12 weeks thereafter. Response is assessed by the investigator using RECIST v1.1 (Eisenhauer et al., Eur J Cancer, 45: 228-247, 2009). Response per modified RECIST v1.1 for immune-based therapeutics (iRECIST) is determined programmatically by the Sponsor on the basis of investigator-assessed individual lesion data (Seymour et al., Lancet Oncol, 18: e143-152, 2017). If clinical activity is demonstrated in an experimental arm, the Sponsor may request that tumor assessment scans for that arm be submitted for evaluation by an independent reading facility.

Baseline tumor tissue samples are collected from all patients, preferably by means of a biopsy performed at study entry. If a biopsy is not deemed feasible by the investigator, archival tumor tissue may be submitted. If deemed clinically feasible by the investigator, tumor tissue is also collected from patients who discontinue Stage 1 because of unacceptable toxicity, disease progression per RECIST v1.1 (control arm), or loss of clinical benefit as determined by the investigator (experimental arms). For patients enrolled in an experimental arm during the expansion phase, an on-treatment tumor tissue sample is collected 4 weeks after initiation of Stage 1 treatment (if clinically feasible), unless on-treatment tissue samples have already been collected, and determined to be evaluable, from a minimum of 15 patients treated with the same CIT combination. These samples, as well as blood samples collected during the study, are utilized for biomarker research.

To characterize the pharmacokinetic (PK) properties and/or immunogenicity of atezolizumab and the other therapeutic agents, blood samples are obtained at various timepoints before and during study treatment administration.

Tumor and Response Evaluations

Patients undergo tumor assessments at baseline, every 6 weeks (±1 week) for the first 12 months following treatment initiation, and every 12 weeks (±2 weeks) thereafter, regardless of dose delays, until radiographic disease progression per RECIST v1.1 except in the case of patients who continue treatment after radiographic disease progression; such patients undergo tumor assessments every 8 weeks (±1 week) until loss of clinical benefit as determined by the investigator.

Thus, tumor assessments are to continue according to schedule for patients who discontinue treatment for reasons other than disease progression or loss of clinical benefit, even if they start a new non-protocol-specified anti-cancer therapy. At the investigator's discretion, tumor assessments may be repeated at any time if progressive disease is suspected.

Baseline tumor assessments for Stage 2 must be performed within 28 days prior to initiation of Stage 2 treatment (i.e., Day 1 of Cycle 1). For patients who are eligible for Stage 2, tumor assessments performed prior to, or at the time of unacceptable toxicity or disease progression per RECIST v1.1 (control arm) or loss of clinical benefit (experimental arms) during Stage 1 may serve as baseline assessments for Stage 2, provided the tumor assessments are performed within 28 days prior to initiation of Stage 2 treatment.

All measurable and evaluable lesions should be assessed and documented at screening (in both Stage 1 and Stage 2). Brain metastases treated with radiotherapy or surgery are not considered measurable or evaluable but are documented as a site of metastatic disease. Tumor assessments performed as standard of care prior to obtaining informed consent and within 28 days prior to initiation of study treatment do not have to be repeated at screening.

Screening assessments must include CT scans (with oral and/or IV contrast) or magnetic resonance imaging (MRI) scans of the chest, abdomen, and pelvis. A spiral CT scan of the chest may be obtained but is not a requirement. If a CT scan with contrast is contraindicated (i.e., in patients with contrast allergy or impaired renal clearance), a non-contrast CT scan of the chest may be performed and MRI scans (with IV contrast, if feasible) of the abdomen and pelvis should be performed. Bone scans and CT scans of the neck should also be performed if clinically indicated. At the investigator's discretion, other methods of assessment of measurable disease according to RECIST v1.1 may be used.

If a CT scan for tumor assessment is performed in a positron emission tomography/CT scanner, the CT acquisition must be consistent with the standards for a full-contrast diagnostic CT scan.

All measurable and/or evaluable lesions identified at baseline should be re-assessed at subsequent tumor evaluations. Brain metastases identified at baseline that have been treated with radiotherapy or surgery are not considered measurable or evaluable unless there is suspected disease progression in the brain (i.e., the patient becomes symptomatic). Thus, subsequent head CT scans are not required unless clinically indicated. The same radiographic procedures used to assess disease sites at screening should be used for subsequent tumor assessments (e.g., the same contrast protocol for CT scans). To facilitate evaluation of response per iRECIST, tumor assessments must be continued after disease progression per RECIST v1.1 for patients who receive treatment beyond progression. This includes continued measurement of target lesions, evaluation of non-target lesions (including monitoring for further worsening of any nontarget lesions that have shown unequivocal progression), and evaluation of any newly identified lesions (including measurements, if lesions are measurable) at all subsequent assessments. Overall response at a single timepoint is assessed by the investigator using RECIST v1.1

Biomarker Assessments

Exploratory biomarker research may include, but is not limited to, analysis of genes or gene signatures associated with tumor molecular subtype and tumor immunobiology, PD-L1, lymphocyte subpopulations, T cell-receptor repertoire, cytokines associated with T-cell activation or density, localization, and activation status of ICs and their subsets, and may involve DNA or RNA extraction, analysis of somatic mutations, and use of next-generation sequencing (NGS) (including whole exome sequencing ((WES)).

Blood samples for biomarker assessments are collected at baseline and during the study. Changes in biomarkers in blood may provide evidence of biologic activity of the specific treatment combinations. Correlations between surrogate biomarkers in blood (such as tumor burden markers, cytokines, chemokines, IC subpopulations, gene expression, and circulating-tumor DNA) and drug dose, efficacy, and safety endpoints may allow for the development of a blood-based biomarker to help define future treatments that may be predictive of which patients are more likely to benefit from specific treatment combinations.

Baseline tumor tissue samples are collected from all patients, preferably by means of a biopsy performed at study entry. Tumor tissue samples collected at baseline are used for determination of PD-L1 and TIGIT expression and for exploratory research on biomarkers. If deemed clinically feasible by the investigator, tumor tissue is collected from patients who discontinue during Stage 1 because of unacceptable toxicity, disease progression per RECIST v1.1, or loss of clinical benefit as determined by the investigator, to enable analysis of tumor tissue biomarkers related to resistance, disease progression, and clinical benefit of study treatment.

If deemed clinically feasible by the investigator, patients enrolled in an experimental arm during the expansion phase undergo an on-treatment biopsy 4 weeks (±7 days) after initiation of Stage 1 treatment, unless on-treatment tissue samples have already been collected, and determined to be evaluable, from a minimum of 15 patients treated with the same CIT combination. On-treatment tissue samples are collected in an effort to better understand potential biological changes that occur during treatment with CIT combinations (including immune escape), provide evidence of pharmacodynamic effects, or confirm hypothesized mechanisms of action.

Tumor samples are evaluated for biomarkers such as tumor-infiltrating ICs, PD-L1, TIGIT, and CD8. Evaluation of the tumor microenvironment in response to treatment within each arm, including changes in the number and functional status of tumor-infiltrating ICs, could provide validation of the postulated mechanism of action and confirmation that an appropriate dose and exposure for the specific treatment combination have been achieved.

Tumor tissue and blood samples may be analyzed through use of NGS, including WES, to identify somatic mutations that are predictive of response to study drug, are associated with progression to a more severe disease state, are associated with acquired resistance to study drug, are associated with susceptibility to developing adverse events, or can increase the knowledge and understanding of disease biology.

### Example 25. Statistical Considerations and Analysis Plan for YO39609 Study

The final study analysis is based on patient data collected through study discontinuation. If not otherwise specified, efficacy analyses are based on the efficacy-evaluable population, defined as all patients who receive at least one dose of each drug for their assigned treatment regimen, and safety analyses are based on the safety-evaluable population, defined as all patients who receive any amount of study treatment.

The analysis results are summarized by the treatment that patients actually received. Results are also summarized by stage (Stage 1 or 2) whenever Stage 2 treatment is available. Data are described and summarized as warranted by sample size. Continuous variables are summarized through use of means, standard deviations, medians, and ranges. Categorical variables are summarized through use of counts and percentages. Listings are used in place of tables in the event of small sample sizes.

New baseline values are established for the Stage 2 efficacy and safety analyses. For evaluation of tumor response, new baseline tumor assessments are established. For other endpoints (e.g., change from baseline in vital signs or laboratory test results), the last non-missing value prior to a patient's first dose during Stage 2 serves as the new baseline.

Determination of Sample Size

The study is not designed to make explicit power and type I error considerations for a hypothesis test. Instead, this study is designed to obtain preliminary efficacy, safety, and PK data on immunotherapy-based treatment combinations when administered to patients with esophageal cancer.

The study enrolls patients with esophageal cancer who have not received prior systemic therapy in this setting. Approximately 100-165 patients are randomly assigned to the control and experimental arms during the study.

G. Efficacy Analyses

The efficacy-evaluable populations are defined as all patients who received at least one dose of each drug for their assigned treatment regimen. Efficacy endpoints are summarized by actual treatment arm.

Primary Efficacy Endpoint

The primary efficacy endpoint is objective response rate (ORR) during Stage 1 based on RECIST v1.1, defined as the proportion of patients with an objective response. An objective response is defined as a complete response or a partial response on two consecutive occasions ≥4 weeks apart, as determined by the investigator using RECIST v1.1. Patients not meeting this criterion, including patients without a post-baseline tumor assessment, are considered to be non-responders. The ORR is summarized, as well as its 95% CI (Clopper-Pearson method). The analysis population for ORR is the efficacy-evaluable population.

Secondary Efficacy Endpoints

The secondary efficacy endpoints are PFS, OS, OS at specific timepoints (e.g., 6 months and 12 months), duration of response (DOR), disease control during Stage 1, objective response in patients with TIGIT-positive tumors, and objective response in patients with PD-L1-positive tumors, as defined in Table 77. PFS, DOR, and disease control are determined by the investigator according to RECIST v1.1.

DOR is derived for efficacy-evaluable patients with a confirmed complete or partial response. For patients who do not have documented disease progression or death in a study stage, PFS and DOR are censored at the day of the last tumor assessment. Patients who are still alive at the time of OS analysis are censored at the last date they were known to be alive.

The Kaplan-Meier method is used to estimate the median for PFS, OS, and DOR, with 90% confidence intervals (CIs) constructed through use of the Brookmeyer and Crowley method. OS rate at specific timepoints is also estimated using the Kaplan-Meier method, with 90% CIs calculated on the basis of Greenwood's estimate for the variance.

Disease control rate, defined as the proportion of patients with stable disease for 16 weeks, a partial response, or a complete response, is calculated for each treatment arm, with 90% CIs estimated through use of Clopper-Pearson's exact method.

Exploratory Efficacy Endpoints

The exploratory efficacy endpoints are objective response, PFS, DOR, and disease control during Stage 1, as determined by the investigator according to iRECIST, and objective response, PFS, DOR, and disease control during Stage 2, as determined by the investigator according to RECIST v1.1 and iRECIST.

H. Safety Analyses

Safety analyses include all patients who received at least one dose of any component of study treatment. Exposure to study treatment is summarized overall and by actual treatment arm within each stage.

Verbatim adverse event terms are mapped to Medical Dictionary for Regulatory Activities thesaurus terms, and adverse event severity is graded according to NCI CTCAE v4.0.

Safety is assessed through summaries of adverse events, changes in laboratory test results, changes in vital signs and ECGs, and exposure to study drugs. Exposure to combination treatment and length of safety follow-up are summarized by treatment arm within each stage.

Treatment-emergent adverse events occurring after initiation of treatment are summarized. For each patient, the maximum reported severity of each adverse event is used in the summaries by severity grade. All treatment-emergent adverse events, serious adverse events, adverse events leading to withdrawal of study treatment, Grade ≥3 adverse events, deaths, and causes of death are listed and summarized by mapped term, appropriate thesaurus level, and NCI CTCAE severity grade.

Relevant laboratory, vital sign (pulse rate, respiratory rate, pulse oximetry, blood pressure, and temperature), and ECG data are displayed by time, with grades identified where appropriate. Additionally, a shift table of selected laboratory tests is used to summarize the baseline and maximum post-baseline severity grade. Changes in vital signs and ECGs are summarized.

I. Pharmacokinetic Analyses

Sparse samples are collected for PK analyses of atezolizumab (patients who receive at least one dose of atezolizumab) and specified drugs given in combination with atezolizumab (patients who receive at least one dose of the drug). Serum or plasma concentrations of the various study drugs are reported as individual values and summarized (mean, standard deviation, coefficient of variation, median, range, geometric mean, and geometric mean coefficient of variation) by treatment arm, and by cycle and day when appropriate and as data allow. Individual and median serum or plasma concentrations of the various study drugs are plotted by treatment arm, cycle, and day. PK data for combination drugs may be compared with available historical data from internal and published previous studies. Atezolizumab concentration data may be pooled with data from other studies using an established population PK model to derive PK parameters such as clearance, volume of distribution, and area under the concentration-time curve.

J. Immunogenicity Analyses

Immunogenicity is assessed for atezolizumab and other study treatments as appropriate. The immunogenicity analyses for atezolizumab include all patients with at least one anti-drug antibody (ADA) assessment. Patients are grouped according to treatment received or, if no treatment is received prior to study discontinuation, according to treatment assigned. For atezolizumab, the number and proportion of ADA-positive patients and ADA-negative patients at baseline (baseline prevalence) and after baseline (postbaseline incidence) are summarized by treatment arm. For other study treatments for which ADAs are tested, ADA positivity is determined according to standard methods established for previous studies of these drugs. The relationship between ADA status and safety, efficacy, PK, and biomarker endpoints may be analyzed and reported using descriptive statistics.

K. Biomarker Analyses

Exploratory biomarker analyses are performed in an effort to understand the association of these biomarkers with response to study drugs, taking into account efficacy and safety endpoints.

L. Interim Analyses

The interim analyses are conducted over the course of the study, with the earliest (Stage 1) interim analysis taking place when at least one experimental arm has completed enrollment in the preliminary phase and patients have been followed for a minimum of 6 weeks. A posterior probability may be used to guide further enrollment in a treatment arm based on an interim analysis of clinical activity in the experimental arm compared with the control arm. If the interim analysis suggests that the activity in an experimental arm is higher than that in the control arm, there may be enrollment of an additional 25 patients in the experimental arm. An interim analysis is also conducted after approximately 15 patients have been enrolled in a Stage 2 treatment arm and followed for a minimum of 6 weeks. If no clinical activity is observed in a Stage 2 treatment arm, further enrollment in that arm is stopped.

### Example 26. PD-L1 as a Predictive Biomarker for Tiragolumab+Atezolizumab Treatment

In the Phase Ib study (GO30103; NCT02794571), tiragolumab was well-tolerated as monotherapy and in combination with atezolizumab in multiple solid tumor types. In the randomized Phase II CITYSCAPE study in 1 L NSCLC (NCT03563716), clinically meaningful improvements were seen in ORR and PFS with tiragolumab+atezolizumab vs placebo+atezolizumab in the intention-to-treat (ITT) population. A greater magnitude of improvement was seen in the PD-L1 tumor proportion score (TPS) ≥50% subgroup (as assessed using the PharmDx 22C3 IHC assay; data cut-off December 2019; median follow-up 10.9 months).

The value of PD-L1 as a predictive biomarker for tiragolumab+atezolizumab treatment was found to be consistent across different PD-L1 assays. IHC was performed to evaluate PD-L1 protein expression for all available patient samples using the pharmDx 22C3 assay (ITT population) and the Conformité Européenne (European Conformity) in vitro diagnostic (CE-IVD0 VENTANA SP263 IHC assay (biomarker evaluable population); the levels of PD-L1 expression were scored using the established algorithms for each assay. Baseline characteristics of the BEP and ITT populations were similar (Table 92).

TABLE 92
Administration of first and subsequent tiragolumab infusions

| | ITT | BEP (SP263)* |
|---|---|---|
| n, (%) | (N = 135) | (n = 113) |
| Age <65 years | 56 (41%) | 49 (43%) |
| Male | 87 (64%) | 75 (66%) |
| White | 82 (61%) | 70 (62%) |
| Asian | 41(30%) | 32 (28%) |
| ECOG PS 0 | 39 (29%) | 29 (26%) |
| Never used tobacco | 14 (10%) | 13 (12%) |
| Non-squamous histology | 80 (59%) | 65 (58%) |
| PD-L1 TPS ≥50% | 58 (43%) | 50 (44%) |
| PD-L1 TPS 1-49% | 77 (57%) | 63 (56%) |
| PD-L1 SP263 TC ≥50% | 45 (33%) | 45 (40%) |
| PD-L1 SP263 TC <50% | 68 (50%) | 68 (60%) |
| PD-L1 SP263 TC missing | 22 (16%) | — |
| TIGIT IHC ≥5% | 49 (36%) | 43 (38%) |
| TIGIT IHC <5% | 56 (41%) | 54 (48%) |
| TIGIT IHC missing | 30 (22%) | 16 (14%) |

*Not all patients had available tissue sample for testing with the SP263 IHC assay.

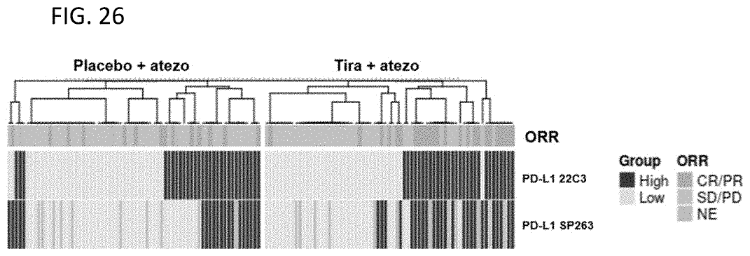

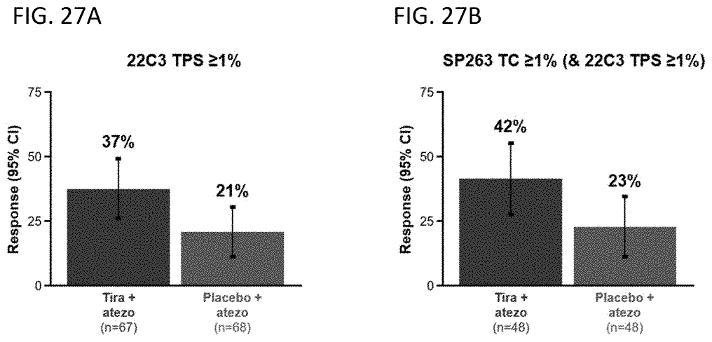

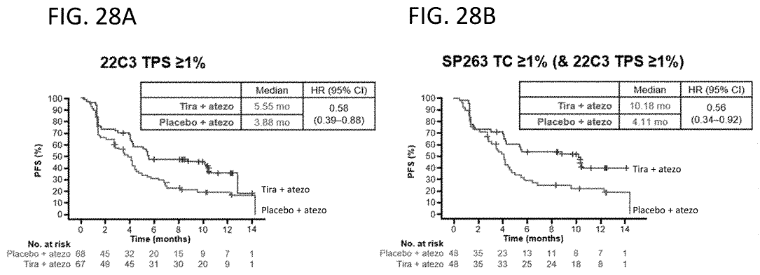

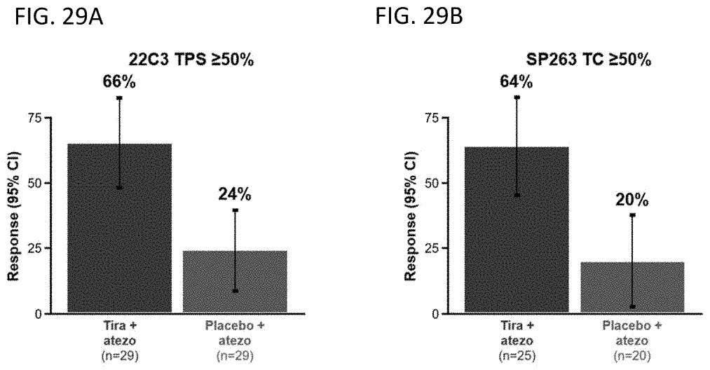

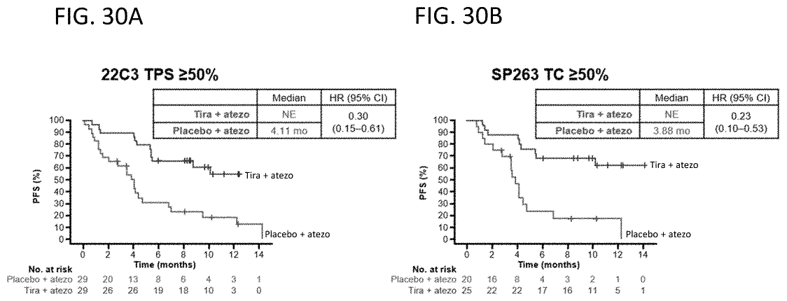

Prevalence of PD-L1 subgroups was comparable between the two IHC assays (FIG. 26). For the PD-L1 22C3 assay, high TPS was classified as TPS ≥50%; low TPS was classified as TPS 1-49%. For the SP263 IHC assay, high TC was classified as TC ≥50%; low TC was classified as TC 1-49%. Comparable ORR and PFS improvements with tiragolumab+atezolizumab vs. atezolizumab monotherapy were seen between the PD-L1-positive (TC ≥1%) subgroup defined by SP263 (PFS HR 0.56, 95% CI: 0.34-0.92) and the PD-L1-positive (TPS ≥1%) subgroup defined by 22C3 (FIGS. 27A, 27B, 28A, and 28B). Comparable ORR and PFS improvements with tiragolumab+atezolizumab vs. atezolizumab monotherapy were also seen between the PD-L1-high (TC ≥50%) subgroup defined by SP263 (PFS HR 0.23, 95% CI: 0.10-0.53) and the PD-L1-high (TPS ≥50%) subgroup defined by 22C3 (FIGS. 29A, 29B, 30A, and 30B).

High PD-L1 expression, assessed either by 22C3 or SP263 IHC, may be an important predictive biomarker for tiragolumab+atezolizumab combination therapy.

### VII. Other Embodiments

Some embodiments of the technology described herein can be defined according to any of the following numbered embodiments:

1. A method of treating a subject having a cancer, the method comprising administering to the subject a dosing regimen comprising one or more dosing cycles of an anti-TIGIT antagonist antibody at a dose of about 500 mg to about 700 mg every three weeks, a PD-1 axis binding antagonist at a dose of about 900 mg to about 1500 mg every three weeks, a platinum-based chemotherapeutic agent every three weeks, and a non-platinum-based chemotherapeutic agent every three weeks.
2. A method of treating a subject having a cancer, the method comprising administering to the subject a dosing regimen comprising one or more dosing cycles of an anti-TIGIT antagonist antibody at a dose of about 700 mg to about 1000 mg every four weeks and a PD-1 axis binding antagonist at a dose of about 1400 mg to 2000 mg every four weeks.
3. A method of treating a subject having a cancer, the method comprising administering to the subject a dosing regimen comprising one or more dosing cycles of an anti-TIGIT antagonist antibody at a dose of about 300 mg to about 600 mg every two weeks and a PD-1 axis binding antagonist at a dose of about 600 mg to about 1200 mg every two weeks.
4. The method of embodiment 2 or 3, wherein the method comprises further administering to the subject one or more chemotherapeutic agents.
5. The method of any one of embodiments 1-4, wherein the anti-TIGIT antagonist antibody is an IgG class antibody, in particular an IgG1 subclass antibody.
6. The method of any one of embodiments 1-5, wherein the anti-TIGIT antagonist antibody comprises the following hypervariable regions (HVRs):
(a) an HVR-H1 sequence comprising the amino acid sequence of SNSAAWN (SEQ ID NO: 1);
(b) an HVR-H2 sequence comprising the amino acid sequence of KTYYRFKWYSDYAVSVKG (SEQ ID NO: 2);
(c) an HVR-H3 sequence comprising the amino acid sequence of ESTTYDLLAGPFDY (SEQ ID NO: 3);
(d) an HVR-L1 sequence comprising the amino acid sequence of KSSQTVLYSSNNKKYLA (SEQ ID NO: 4);
(e) an HVR-L2 sequence comprising the amino acid sequence of WASTRES (SEQ ID NO: 5); and
(f) an HVR-L3 sequence comprising the amino acid sequence of QQYYSTPFT (SEQ ID NO: 6).
7. The method of any one of embodiments 1-6, wherein the anti-TIGIT antagonist antibody further comprises the following light chain variable region framework regions (FRs):
(a) an FR-L1 comprising the amino acid sequence of DIVMTQSPDSLAVSLGERATINC (SEQ ID NO: 7);
(b) an FR-L2 comprising the amino acid sequence of WYQQKPGQPPNLLIY (SEQ ID NO: 8);
(c) an FR-L3 comprising the amino acid sequence of GVPDRFSGSGSGTDFTLTISSLQAEDVAVYYC (SEQ ID NO: 9); and
(d) an FR-L4 comprising the amino acid sequence of FGPGTKVEIK (SEQ ID NO: 10), and
the following heavy chain variable region FRs:
(a) an FR-H1 comprising the amino acid sequence of XiVQLQQSGPGLVKPSQTLSLTCAISGDSVS (SEQ ID NO: 11), wherein X1 is E or Q;
(b) an FR-H2 comprising the amino acid sequence of WIRQSPSRGLEWLG (SEQ ID NO: 12);
(c) an FR-H3 comprising the amino acid sequence of RITINPDTSKNQFSLQLNSVTPEDTAVFYCTR (SEQ ID NO: 13); and
(d) an FR-H4 comprising the amino acid sequence of WGQGTLVTVSS (SEQ ID NO: 14).
8. The method of any one of embodiments 1-7, wherein the anti-TIGIT antagonist antibody comprises:
(a) a heavy chain variable (VH) domain comprising an amino acid sequence having at least 95% sequence identity to the amino acid sequence of SEQ ID NO: 17 or 18;
(b) a light chain variable (VL) domain comprising an amino acid sequence having at least 95% sequence identity to the amino acid sequence of SEQ ID NO: 19; or
(c) a VH domain comprising the amino acid sequence of SEQ ID NO: 17 or 18 and a VL domain comprising the amino acid sequence of SEQ ID NO: 19.
9. The method of any one of embodiments 1-8, wherein the anti-TIGIT antagonist antibody is tiragolumab.
10. The method of any one of embodiments 1-9, wherein the PD-1 axis binding antagonist is a PD-L1 binding antagonist or a PD-1 binding antagonist.
11. The method of any one of embodiments 1-10, wherein the PD-1 axis binding antagonist is an anti-PD-L1 antagonist antibody or an anti-PD-1 antagonist antibody.
12. The method of any one of embodiments 1-11, wherein the PD-1 axis binding antagonist is an anti-PD-L1 antagonist antibody.
13. The method of embodiment 12, wherein the anti-PD-L1 antagonist antibody atezolizumab, MDX-1105, durvalumab, avelumab, SHR-1316, CS1001, envafolimab, TQB2450, ZKAB001, LP-002, CX-072, IMC-001, KL-A167, APL-502, cosibelimab, lodapolimab, FAZ053, TG-1501, BGB-A333, BCD-135, AK-106, LDP, GR1405, HLX20, MSB2311, RC98, PDL-GEX, KD036, KY1003, YBL-007, or HS-636.
14. The method of any one of embodiments 1-13, wherein the PD-1 axis binding antagonist is atezolizumab.
15. The method of any one of embodiments 1-11, wherein the PD-1 axis binding antagonist is an anti-PD-1 antagonist antibody.
16. The method of embodiment 15, wherein the anti-PD-1 antagonist antibody is nivolumab, pembrolizumab, MEDI-0680, spartalizumab, cemiplimab, BGB-108, prolgolimab, camrelizumab, sintilimab, tislelizumab, toripalimab, dostarlimab, retifanlimab, sasanlimab, penpulimab, CS1003, HLX10, SCT-110A, zimberelimab, balstilimab, genolimzumab, BI 754091, cetrelimab, YBL-006, BAT1306, HX008, budigalimab, AMG 404, CX-188, JTX-4014, 609A, Sym021, LZM009, F520, SG001, AM0001, ENUM 244C8, ENUM 388D4, STI-1110, AK-103, or hAb21.
17. The method of any one of embodiments 2-16, wherein the method comprises administering to the subject an effective amount of a platinum-based chemotherapeutic agent and a non-platinum-based chemotherapeutic agent.
18. The method of embodiment 1 or 17, wherein the non-platinum-based chemotherapeutic agent is a topoisomerase II inhibitor.
19. The method of embodiment 18, wherein the topoisomerase II inhibitor is etoposide, teniposide, doxorubicin, daunorubicin, mitoxantrone, amsacrine, an ellipticine, aurintricarboxylic acid, or HU-331, in particular etoposide.
20. The method of embodiment 18 or 19, wherein the topoisomerase II inhibitor is administered at a dose of 100 mg/m2.
21. The method of any one of embodiments 17-20, wherein the platinum-based chemotherapeutic agent is carboplatin or cisplatin, in particular carboplatin.
22. The method of any one of embodiments 17-21, wherein the platinum-based chemotherapeutic agent is administered at a dose sufficient to achieve AUC=5 mg/ml/min on the day of administration.
23. The method of any one of embodiments 1-22, wherein the method comprises a dosing regimen comprising an induction phase and a maintenance phase.
24. The method of embodiment 23, wherein the induction phase comprises four initial dosing cycles and wherein the anti-TIGIT antagonist antibody, PD-1 axis binding antagonist, platinum-based chemotherapeutic agent, and non-platinum-based chemotherapeutic agent are administered in each of four initial dosing cycles.
25. The method of embodiment 23 or 24, wherein the maintenance phase does not comprise administration of the platinum-based chemotherapeutic agent and/or non-platinum based chemotherapeutic agent.
26. The method of any one of embodiments 1-25, wherein the treatment extends progression-free survival (PFS) of the subject as compared to treatment without the anti-TIGIT antagonist antibody.
27. The method of any one of embodiments 1-26, wherein the treatment extends overall survival of the subject as compared to treatment without the anti-TIGIT antagonist antibody.
28. The method of any one of embodiments 1-27, wherein the cancer is lung cancer.
29. The method of embodiment 28, wherein the lung cancer is a small cell lung cancer (SCLC), in particular extensive stage SCLC (ES-SCLC).
30. The method of embodiment 28, wherein the lung cancer is a non-small cell lung cancer (NSCLC), in particular locally advanced unresectable NSCLC (Stage IIIB NSCLC).
31. The method of embodiment 28, wherein the lung cancer is locally advanced unresectable or metastatic non-squamous NSCLC.
32. The method of embodiment 28, wherein the lung cancer is resectable lung cancer.
33. The method of any one of embodiments 1-27, wherein the cancer is cervical cancer.
34. The method of any one of embodiments 1-27, wherein the cancer is an early triple-negative breast cancer (eTNBC).
35. The method of any one of embodiments 1-27, wherein the cancer is a head and neck cancer.
36. The method of any one of embodiments 1-27, wherein the cancer is a liver cancer, in particular hepatocellular carcinoma (HCC).
37. The method of any one of embodiments 1-27, wherein the cancer is a bladder cancer.
38. The method of embodiment 37, wherein the cancer is a urothelial carcinoma (UC), in particular a metastatic urothelial carcinoma (mUC).
39. The method of any one of embodiments 1-27, wherein the cancer is a pancreatic cancer, in particular a pancreatic ductal adenocarcinoma (PDAC).
40. The method of any one of embodiments 1-27, wherein the cancer is an esophageal cancer, in particular an advanced or metastatic esophageal cancer.
41. A kit comprising an anti-TIGIT antagonist antibody for use in combination with a PD-1 axis binding antagonist for treating a subject having a cancer according to the method of any one of embodiments 1-40.
42. The kit of embodiment 41, wherein the kit further comprises one or more chemotherapeutic agents.
43. An anti-TIGIT antagonist antibody and a PD-1 axis binding antagonist for use in a method of treating a subject having a cancer, wherein the method is according to any one of embodiments 1-40.
44. Use of an anti-TIGIT antagonist antibody in the manufacture of a medicament for treating a subject having a cancer in combination with a PD-1 axis binding antagonist, wherein the treatment is according to the method of any one of embodiments 1-40.
45. The use of embodiment 44, wherein the anti-TIGIT antagonist antibody and the PD-1 axis binding antagonist are provided in separate formulations.
46. The use of embodiment 44, wherein the anti-TIGIT antagonist antibody and the PD-1 axis binding antagonist are provided in a single formulation.
47. A method of treating a subject having a cancer, the method comprising administering to the subject a dosing regimen comprising one or more dosing cycles of an anti-TIGIT antagonist antibody at a dose of about 200 mg to about 2000 mg every four weeks and a PD-1 axis binding antagonist at a dose of about 80 mg to about 2000 mg every four weeks.
48. A method of treating a subject having a cancer, the method comprising administering to the subject a dosing regimen comprising one or more dosing cycles of an anti-TIGIT antagonist antibody at a dose of about 200 mg to about 2000 mg every four weeks and a PD-1 axis binding antagonist at a dose of about 20 mg to about 1600 mg every two weeks.
49. A method of treating a subject having a cancer, the method comprising administering to the subject a dosing regimen comprising one or more dosing cycles of an anti-TIGIT antagonist antibody at a dose of about 10 mg to about 1000 mg every two weeks and a PD-1 axis binding antagonist at a dose of about 20 mg to about 1600 mg every two weeks.
50. A method of treating a subject having a cancer, the method comprising administering to the subject a dosing regimen comprising one or more dosing cycles of an anti-TIGIT antagonist antibody at a dose of about 10 mg to about 1000 mg every two weeks and a PD-1 axis binding antagonist at a dose of about 80 mg to about 2000 mg every four weeks.
51. A method of treating a subject having a cancer, the method comprising administering to the subject a dosing regimen comprising one or more dosing cycles of an anti-TIGIT antagonist antibody at a dose of about 30 mg to about 1200 mg every three weeks and a PD-1 axis binding antagonist at a dose of about 100 mg to about 1000 mg every six weeks.
52. The method of any one of embodiments 47-51, wherein the method comprises further administering to the subject one or more chemotherapeutic agents.
53. The method of embodiment 52, wherein the method comprises administering to the subject a platinum-based chemotherapeutic agent and a non-platinum-based chemotherapeutic agent.
54. A method of treating a subject having a cancer, the method comprising administering to the subject a dosing regimen comprising one or more dosing cycles of an anti-TIGIT antagonist antibody at a dose of about 30 mg to about 1200 mg every three weeks, a PD-1 axis binding antagonist at a dose of about 80 mg to about 1600 mg every three weeks, a platinum-based chemotherapeutic agent every three weeks, and a non-platinum-based chemotherapeutic agent every three weeks.
55. The method of embodiment 53 or 54, wherein the method comprises an induction phase and a maintenance phase.
56. The method of embodiment 55, wherein the induction phase and maintenance phase each comprise one or more dosing cycles.
57. The method of embodiment 55 or 56, wherein the maintenance phase does not comprise administration of the platinum-based chemotherapeutic agent.
58. The method of any one of embodiments 55-57, wherein the maintenance phase does not comprise administration of the non-platinum-based chemotherapeutic agent.
59. The method of embodiment 58, wherein the maintenance phase comprises one or more dosing cycles of an anti-TIGIT antagonist antibody at a dose of about 200 mg to about 2000 mg every four weeks and a PD-1 axis binding antagonist at a dose of about 80 mg to about 2000 mg every four weeks.
60. A method of treating a subject having a cancer, the method comprising administering to the subject a dosing regimen comprising one or more dosing cycles of an anti-TIGIT antagonist antibody at a dose of about 30 mg to about 1200 mg every three weeks and an anti-PD-1 antagonist antibody at a dose of about 80 mg to about 1600 mg every three weeks, wherein the anti-PD-1 antagonist antibody is pembrolizumab.
61. A method of treating a subject having a cancer, the method comprising administering to the subject a dosing regimen comprising one or more dosing cycles of tiragolumab and pembrolizumab, wherein the pembrolizumab is administered at a dose between about 100 mg to about 1000 mg every six weeks.
62. A method for treating a subject having a cancer, the method comprising administering to the subject a dosing regimen comprising one or more dosing cycles of an anti-TIGIT antagonist antibody at a dose of between about 30 mg to about 1200 mg every three weeks, a PD-1 axis binding antagonist at a dose of between about 80 mg to about 1600 mg every three weeks, and an antimetabolite at a dose of between about 10 mg/m2 to about 10000 mg/m2 twice a day orally every three weeks for 2-weeks on/1-week off.
63. A method of treating a subject having a cancer, the method comprising administering to the subject a dosing regimen comprising one or more dosing cycles of an anti-TIGIT antagonist antibody at a dose of about 30 mg to about 1200 mg every three weeks, a PD-1 axis binding antagonist at a dose of about 80 mg to about 1600 mg every three weeks, gemcitabine, and nab-paclitaxel.
64. The method of embodiment 52, wherein the one or more chemotherapeutic agents are gemcitabine and nab-paclitaxel.
65. A method for treating a subject having a cancer, the method comprising administering to the subject a dosing regimen comprising one or more dosing cycles of an anti-TIGIT antagonist antibody at a dose of between about 30 mg to about 1200 mg every three weeks, a PD-1 axis binding antagonist at a dose of between about 80 mg to about 1600 mg every three weeks, and a VEGF antagonist at a dose of between about 1 mg/kg to about 35 mg/kg every three weeks.
66. A method of treating a subject having a cancer, the method comprising administering to the subject a dosing regimen comprising an induction phase and a maintenance phase, wherein:
(a) the induction phase comprises one or more dosing cycles of an anti-TIGIT antagonist antibody at a dose of about 30 mg to about 1200 mg every three weeks, a PD-1 axis binding antagonist at a dose of about 80 mg to about 1600 mg every three weeks, a platinum-based chemotherapeutic agent every three weeks, and a non-platinum-based chemotherapeutic agent every three weeks; and
(b) the maintenance phase comprises one or more additional dosing cycles of the anti-TIGIT antagonist antibody every three weeks, the PD-1 axis binding antagonist every three weeks, and the non-platinum-based chemotherapeutic agent every three weeks, and wherein the maintenance phase does not comprise administration of the platinum-based chemotherapeutic agent.
67. A method of treating a subject having a cancer, the method comprising administering to the subject a dosing regimen comprising an induction phase and a maintenance phase, wherein:
(a) the induction phase comprises one or more dosing cycles of an anti-TIGIT antagonist antibody at a dose of about 30 mg to about 1200 mg every three weeks, a PD-1 axis binding antagonist at a dose of about 80 mg to about 1600 mg every three weeks, a platinum-based chemotherapeutic agent every three weeks, and a non-platinum-based chemotherapeutic agent every three weeks; and
(b) the maintenance phase comprises one or more additional dosing cycles of the anti-TIGIT antagonist antibody at a dose of about 200 mg to about 2000 mg every four weeks and the PD-1 axis binding antagonist at a dose of about 80 mg to about 2000 mg every four weeks, wherein the maintenance phase does not comprise administration of the platinum-based chemotherapeutic agent or non-platinum-based chemotherapeutic agent.
68. The method of any one of embodiments 56-59, 66, and 67, wherein the induction phase comprises four to six dosing cycles.
69. The method of any one of embodiments 53-59 and 66-68, wherein:
(a) the platinum-based chemotherapeutic agent is carboplatin or cisplatin and the non-platinum-based chemotherapeutic agent is pemetrexed;
(b) the platinum-based chemotherapeutic agent is carboplatin and the non-platinum-based chemotherapeutic agent is paclitaxel; or
(c) the platinum-based chemotherapeutic agent is carboplatin or cisplatin and the non-platinum-based chemotherapeutic agent is etoposide.
70. The method of any one of embodiments 47-69, wherein the cancer is a solid tumor.
71. The method of any one of embodiments 47-70, wherein the cancer is locally advanced or metastatic.
72. The method of any one of embodiments 47-71, wherein the cancer is a lung cancer, a pancreatic cancer, a cervical cancer, a breast cancer, a head and neck cancer, a liver cancer, a bladder cancer, an esophageal cancer, a gastric cancer, a colorectal cancer, a kidney cancer, a renal cancer, a melanoma, or an ovarian cancer.
73. The method of embodiment 72, wherein the cancer is a lung cancer.
74. The method of embodiment 73, wherein the lung cancer is an NSCLC, in particular a locally advanced unresectable NSCLC.
75. The method of embodiment 74, wherein the NSCLC is a Stage IIIB NSCLC.
76. The method of any one of embodiments 73-75, wherein the lung cancer is a recurrent or metastatic NSCLC.
77. The method of embodiment 76, wherein the NSCLC is a Stage IV NSCLC.
78. The method of embodiment 77, wherein the subject has not been previously treated for Stage IV NSCLC.
79. The method of embodiment 73, wherein the lung cancer is a small cell lung cancer (SCLC).
80. A method of treating a subject or population of subjects having a lung cancer, the method comprising administering to the subject or population of subjects a dosing regimen comprising one or more dosing cycles of an effective amount of an anti-TIGIT antagonist antibody, a PD-1 axis binding antagonist, a platinum-based chemotherapeutic agent, and a topoisomerase II inhibitor, wherein the treatment extends progression-free survival (PFS) of the subject as compared to treatment with the PD-1 axis binding antagonist, the platinum-based chemotherapeutic agent, and the topoisomerase II inhibitor without the anti-TIGIT antagonist antibody.
81. A method of treating a population of subjects having a lung cancer, the method comprising administering to the population of subjects a dosing regimen comprising one or more dosing cycles of an effective amount of an anti-TIGIT antagonist antibody, a PD-1 axis binding antagonist, a platinum-based chemotherapeutic agent, and a topoisomerase II inhibitor, wherein the treatment results in a median PFS of the population of subjects of about 8.2 months to about 9.2 months.
82. The method of embodiment 80 or 81, wherein the treatment extends overall survival (OS) of the subject or population of subjects as compared to treatment with the PD-1 axis binding antagonist, the platinum-based chemotherapeutic agent, and the topoisomerase II inhibitor without the anti-TIGIT antagonist antibody.
83. The method of any one of embodiments 80-82, wherein the treatment extends the PFS of the subject or population of subjects by at least about 2.4 months as compared to treatment with the PD-1 axis binding antagonist, the platinum-based chemotherapeutic agent, and the topoisomerase II inhibitor without the anti-TIGIT antagonist antibody.
84. The method of embodiment 83, wherein the treatment extends the PFS of the subject or population of subjects by at least about 3 months to about 4 months as compared to treatment with the PD-1 axis binding antagonist, the platinum-based chemotherapeutic agent, and the topoisomerase II inhibitor without the anti-TIGIT antagonist antibody.
85. A method of treating a subject or population of subjects having a lung cancer, the method comprising administering to the subject or population of subjects a dosing regimen comprising one or more dosing cycles of an effective amount of an anti-TIGIT antagonist antibody, a PD-1 axis binding antagonist, a platinum-based chemotherapeutic agent, and a topoisomerase II inhibitor, wherein the treatment extends OS of the subject as compared to treatment with the PD-1 axis binding antagonist, the platinum-based chemotherapeutic agent, and the topoisomerase II inhibitor without the anti-TIGIT antagonist antibody.
86. A method of treating a population of subjects having a lung cancer, the method comprising administering to the population of subjects a dosing regimen comprising one or more dosing cycles of an effective amount of an anti-TIGIT antagonist antibody, a PD-1 axis binding antagonist, a platinum-based chemotherapeutic agent, and a topoisomerase II inhibitor, wherein the treatment results in a median OS of the population of subjects of about 15.3 months to about 17.6 months.
87. The method of any one of embodiments 82-86, wherein the treatment extends the OS of the subject or population of subjects by at least about 3.3 months as compared to treatment with the PD-1 axis binding antagonist, the platinum-based chemotherapeutic agent, and the topoisomerase II inhibitor without the anti-TIGIT antagonist antibody.
88. The method of any one of embodiments 82-86, wherein the treatment extends the OS of the subject or population of subjects by at least about 3 months to about 5.3 months as compared to treatment with the PD-1 axis binding antagonist, the platinum-based chemotherapeutic agent, and the topoisomerase II inhibitor without the anti-TIGIT antagonist antibody.
89. The method of any one of embodiments 80-88, wherein the anti-TIGIT antagonist antibody, PD-1 axis binding antagonist, platinum-based chemotherapeutic agent, and topoisomerase II inhibitor are administered in each of four initial dosing cycles.
90. The method of any one of embodiments 80-89, wherein the anti-TIGIT antagonist antibody and the PD-1 axis binding antagonist are further administered in one or more additional cycles following the fourth initial dosing cycle.
91. The method of embodiment 90, wherein the platinum-based chemotherapeutic agent and the topoisomerase II inhibitor are omitted from each of the one or more additional dosing cycles.
92. The method of any one of embodiments 80-91, wherein the platinum-based chemotherapeutic agent is carboplatin and the topoisomerase II inhibitor is etoposide.
93. The method of any one of embodiments 80-92, wherein the lung cancer is a small cell lung cancer (SCLC).
94. The method of embodiment 93, wherein the SCLC is extensive stage SCLC (ES-SCLC).
95. The method of embodiment 94, wherein the subject or subjects are treatment-naïve for ES-SCLC.
96. The method of any one of embodiments 80-95, wherein the subject or subjects do not have a presence or history of brain metastases.
97. The method of any one of embodiments 80-96, wherein the lung cancer is unselected for PD-L1 expression.
98. The method of any one of embodiments 80-96, wherein the lung cancer is selected for PD-L1 expression.
99. The method of embodiment 98, wherein the lung cancer is selected for PD-L1 expression by a detectable expression level of PD-L1.
100. The method of any one of embodiments 80-99, wherein the lung cancer is metastatic.
101. The method of embodiment 100, wherein the lung cancer has metastasized to the brain, liver, lymph nodes, and/or adrenal gland.
102. The method of embodiment 100 or 101, wherein the lung cancer has not metastasized to the brain.
103. The method of any one of embodiments 100-102, wherein the treatment results in a complete response (CR) or a partial response (PR).
104. The method of any one of embodiments 80-103, wherein the anti-TIGIT antagonist antibody is administered at a dose of about 30 mg to about 1200 mg every two weeks, three weeks, or four weeks.
105. The method of any one of embodiments 80-104, wherein the PD-1 axis binding antagonist is administered at a dose of about 80 mg to about 2000 mg every two weeks, three weeks, or four weeks.
106. The method of any one of embodiments 80-105, wherein the PD-1 axis binding antagonist, the anti-TIGIT antagonist antibody, the platinum-based chemotherapeutic agent, and the topoisomerase II inhibitor are administered sequentially.
107. The method of embodiment 106, wherein the PD-1 axis binding antagonist is administered prior to the anti-TIGIT antagonist antibody, the anti-TIGIT antagonist antibody is administered prior to the platinum-based chemotherapeutic agent, and the platinum agent is administered prior to the topoisomerase II inhibitor.
108. The method of any one of embodiments 80-107, wherein the anti-TIGIT antagonist antibody, PD-1 axis binding antagonist, the platinum-based chemotherapeutic agent, and the topoisomerase II inhibitor are administered intravenously.
109. The method of any one of embodiments 80-108, wherein one or more of the anti-TIGIT antagonist antibody, the PD-1 axis binding antagonist, the platinum-based chemotherapeutic agent, and the topoisomerase II inhibitor are administered subcutaneously.
110. A method for treating a subject having SCLC, the method comprising administering to the subject one or more 21-day dosing cycles of an anti-TIGIT antagonist antibody at a dose of about 30 mg to about 1200 mg on Day 1 of each dosing cycle, atezolizumab at a dose of about 80 mg to about 1600 mg on Day 1 of each dosing cycle, carboplatin at a dose sufficient to achieve AUC=5 mg/ml/min on Day 1 of each dosing cycle, and etoposide at a dose of 100 mg/m2 on each of Days 1, 2, and 3 of each dosing cycle, wherein the treatment extends PFS and/or OS of the subject as compared to treatment with atezolizumab, carboplatin, and etoposide without the anti-TIGIT antagonist antibody.
111. A method for treating a subject having SCLC, the method comprising administering to the subject one or more 21-day dosing cycles of an anti-TIGIT antagonist antibody at a dose of about 300 mg to about 800 mg on Day 1 of each dosing cycle, atezolizumab at a dose of about 900 mg to about 1500 mg on Day 1 of each dosing cycle, carboplatin at a dose sufficient to achieve AUC=5 mg/ml/min on Day 1 of each dosing cycle, and etoposide at a dose of 100 mg/m2 on each of Days 1, 2, and 3 of each dosing cycle, wherein the treatment extends PFS and/or OS of the subject as compared to treatment with atezolizumab, carboplatin, and etoposide without the anti-TIGIT antagonist antibody.
112. The method of embodiment 110 or 111, further comprising administering to the subject one or more additional 21-day dosing cycles of the anti-TIGIT antagonist antibody at a dose of about 30 mg to about 1200 mg on Day 1 of each additional dosing cycle and atezolizumab at a dose of about 80 mg to about 1600 mg on Day 1 of each additional dosing cycle, wherein carboplatin and etoposide are omitted from each of the one or more additional dosing cycles.
113. A method for treating a subject or population of subjects having ES-SCLC, the method comprising administering to the subject or population of subjects four initial dosing cycles followed by one or more additional dosing cycles, wherein:
(a) the four initial dosing cycles comprise administering tiragolumab at a dose of about 600 mg on Day 1 of each initial dosing cycle, atezolizumab at a dose of about 1200 mg on Day 1 of each initial dosing cycle, carboplatin at a dose sufficient to achieve AUC=5 mg/ml/min on Day 1 of each initial dosing cycle, and etoposide at a dose of 100 mg/m2 on each of Days 1, 2, and 3 of each initial dosing cycle; and
(b) the one or more additional dosing cycles comprise administering tiragolumab at a dose of about 600 mg on Day 1 of each additional dosing cycle and atezolizumab at a dose of about 1200 mg on Day 1 of each additional dosing cycle, wherein the four initial dosing cycles and the one or more additional dosing cycles are each 21-day dosing cycles, and wherein the treatment extends PFS and/or OS of the subject or population of subjects as compared to treatment with atezolizumab, carboplatin, and etoposide without the tiragolumab.
114. A method of treating a subject or population of subjects having a lung cancer, the method comprising administering to the subject or population of subjects a dosing regimen comprising one or more dosing cycles of an anti-TIGIT antagonist antibody, a PD-1 axis binding antagonist, a first chemotherapeutic agent which is a platinum-based chemotherapeutic agent, and a second chemotherapeutic agent which is a non-platinum-based chemotherapeutic agent.
115. The method of embodiment 114, wherein the lung cancer has not been evaluated for PD-L1 expression.
116. The method of embodiment 114 or 115, wherein the subject or subjects have not been determined to have a PD-L1-positive tumor cell fraction of greater than, or equal to, 50%.
117. The method of embodiment 116, wherein the subject or subjects have been determined to have a PD-L1-positive tumor cell fraction of less than 50%.
118. The method of embodiment 116 or 117, wherein the PD-L1-positive tumor cell fraction is determined by positive staining with an anti-PD-L1 antibody, wherein the anti-PD-L1 antibody is SP263 or 22C3.
119. The method of any one of embodiments 114-118, wherein the subject or subjects do not have an epidermal growth factor receptor (EGFR) or anaplastic lymphoma kinase (ALK) genomic tumor aberration.
120. The method of any one of embodiments 114-119, wherein the subject or subjects have received no prior systemic therapy for the lung cancer.
121. The method of any one of embodiments 114-120, wherein the lung cancer is a locally advanced lung cancer.
122. The method of any one of embodiments 114-121, wherein the lung cancer is an NSCLC, in particular a locally advanced unresectable or metastatic non-squamous NSCLC.
123. The method of embodiment 122, wherein the non-squamous NSCLC is a Stage IV non-squamous NSCLC.
124. The method of any one of embodiments 114-123, wherein the dosing regimen comprises an induction phase comprising four dosing cycles, and wherein the anti-TIGIT antagonist antibody, the PD-1 axis binding antagonist, the platinum-based chemotherapeutic agent, and the non-platinum-based chemotherapeutic agent are administered on Day 1 of each dosing cycle of the induction phase.
125. The method of embodiment 124, wherein the dosing regimen comprises a maintenance phase following the induction phase, wherein the maintenance phase comprises one or more dosing cycles, and wherein the anti-TIGIT antagonist antibody, the PD-1 axis binding antagonist, and the non-platinum-based chemotherapeutic agent are administered on Day 1 of each dosing cycle of the maintenance phase.
126. The method of embodiment 125, wherein the one or more dosing cycles of the maintenance phase do not comprise administration of the platinum-based chemotherapeutic agent.
127. The method of any one of embodiments 114-126, wherein the platinum-based chemotherapeutic agent is carboplatin or cisplatin and the non-platinum-based chemotherapeutic agent is pemetrexed.
128. A method of treating a subject or population of subjects having an advanced non-squamous NSCLC, the method comprising administering to the subject or population of subjects a dosing regimen comprising four 21-day dosing cycles of tiragolumab, atezolizumab, carboplatin or cisplatin, and pemetrexed, wherein the tiragolumab is administered at a dose of about 600 mg every three weeks, the atezolizumab is administered at a dose of about 1200 mg every three weeks, the carboplatin is administered at a dose sufficient to achieve an AUC=5 mg/ml/min every three weeks or the cisplatin is administered at a dose of 75 mg/m2 every three weeks, and the pemetrexed is administered at a dose of about 500 mg/m2 every three weeks on Day 1 of each of the four 21-day dosing cycles.
129. A method of treating a subject or population of subjects having an advanced non-squamous NSCLC, the method comprising administering to the subject or population of subjects:
(i) four induction phase dosing cycles of tiragolumab at a dose of about 600 mg every three weeks, atezolizumab at a dose of about 1200 mg every three weeks, carboplatin at a dose sufficient to achieve an AUC=5 mg/ml/min every three weeks, and pemetrexed at a dose of about 500 mg/m2 every three weeks; and
(ii) one or more maintenance phase dosing cycles of tiragolumab at a dose of about 600 mg every three weeks, atezolizumab at a dose of about 1200 mg every three weeks, and pemetrexed at a dose of about 500 mg/m2 every three weeks, wherein the one or more 21-day dosing cycles of the maintenance phase do not comprise administration of the carboplatin,
wherein the subject or population of subjects have received no prior systemic therapy for the advanced non-squamous NSCLC.
130. A method of treating a subject having an advanced non-squamous NSCLC, the method comprising administering to the subject:
(i) four induction phase dosing cycles of tiragolumab at a dose of about 600 mg every three weeks, atezolizumab at a dose of about 1200 mg every three weeks, carboplatin at a dose sufficient to achieve an AUC=5 mg/ml/min every three weeks, and pemetrexed at a dose of about 500 mg/m2 every three weeks; and
(ii) one or more maintenance phase dosing cycles of tiragolumab at a dose of about 600 mg every three weeks, atezolizumab at a dose of about 1200 mg every three weeks, and pemetrexed at a dose of about 500 mg/m2 every three weeks, wherein the one or more 21-day dosing cycles of the maintenance phase do not comprise administration of the carboplatin wherein the subject has received no prior systemic therapy for the advanced non-squamous NSCLC.
131. A method of treating a subject having an advanced non-squamous NSCLC, the method comprising administering to the subject a dosing regimen comprising four 21-day dosing cycles of tiragolumab, atezolizumab, carboplatin or cisplatin, and pemetrexed, wherein the tiragolumab is administered at a dose of about 600 mg every three weeks, the atezolizumab is administered at a dose of about 1200 mg every three weeks, the cisplatin is administered at a dose of 75 mg/m2, and the pemetrexed is administered at a dose of about 500 mg/m2 on Day 1 of each of the four 21-day dosing cycles.
132. The method of any one of embodiments 114-131, wherein the treatment extends the PFS of the subject or population of subjects by at least about 3.5 months or about 4.7 months.
133. The method of any one of embodiments 114-132, wherein the treatment results in a median PFS of the population of subjects of about 12.5 months to about 14.7 months.
134. The method of any one of embodiments 114-133, wherein the treatment extends the OS of the subject or population of subjects by at least about 5.5 months or about 8.0 months.
135. The method of any one of embodiments 114-134, wherein the treatment results in a median OS of the population of subjects of about 27.5 months to about 32.0 months.
136. A method for treating a subject having a resectable lung cancer, the method comprising administering to the subject one or more dosing cycles of an anti-TIGIT antagonist antibody at a dose of between about 30 mg to about 1200 mg every three weeks and a PD-1 axis binding antagonist at a dose of between about 80 mg to about 1600 mg every three weeks.
137. A method for treating a subject having a resectable lung cancer, the method comprising administering to the subject one or more dosing cycles of an anti-TIGIT antagonist antibody at a dose of between about 300 mg to about 800 mg every three weeks and a PD-1 axis binding antagonist at a dose of between about 900 mg to about 1500 mg every three weeks.
138. A method for treating a subject having a lung cancer, the method comprising administering to the subject one or more dosing cycles of an anti-TIGIT antagonist antibody and a PD-1 axis binding antagonist, wherein at least one of the dosing cycles comprises administering to the subject the anti-TIGIT antagonist antibody at a dose of between about 30 mg to about 1200 mg every three weeks and the PD-1 axis binding antagonist at a dose of between about 80 mg to about 1600 mg every three weeks as a neoadjuvant treatment.
139. A method for treating a subject having a lung cancer, the method comprising administering to the subject one or more dosing cycles of an anti-TIGIT antagonist antibody and a PD-1 axis binding antagonist, wherein at least one of the dosing cycles comprises administering to the subject the anti-TIGIT antagonist antibody at a dose of between about 300 mg to about 800 mg every three weeks and the PD-1 axis binding antagonist at a dose of between about 900 mg to about 1500 mg every three weeks as a neoadjuvant treatment.
140. The method of embodiment 138 or 139, wherein at least one of the dosing cycles comprises administering to the subject the anti-TIGIT antagonist antibody at a dose of between about 30 mg to about 1200 mg every three weeks and the PD-1 axis binding antagonist at a dose of between about 80 mg to about 1600 every three weeks as an adjuvant treatment.
141. The method of embodiment 140, wherein at least one of the dosing cycles comprises administering to the subject the anti-TIGIT antagonist antibody at a dose of between about 500 mg to about 700 mg every three weeks and the PD-1 axis binding antagonist at a dose of between about 900 mg to about 1500 every three weeks as an adjuvant treatment.
142. The method of any one of embodiments 138-141, wherein the lung cancer is a resectable lung cancer.
143. The method of any one of embodiments 136-142, wherein the lung cancer is an early stage lung cancer.
144. The method of any one of embodiments 136-143, wherein the lung cancer is a stage II, IIIA, or IIIB lung cancer.
145. The method of any one of embodiments 136-144, wherein the lung cancer does not have an epidermal growth factor receptor (EGFR) or anaplastic lymphoma kinase (ALK) genomic tumor aberration.
146. The method of any one of embodiments 136-145, wherein the subject is eligible for an R0 resection with curative intent.
147. The method of any one of embodiments 136-146, wherein the subject has not received a prior therapy for lung cancer.
148. The method of embodiment 147, wherein the prior therapy is an immunotherapy, a chemotherapy, or a radiotherapy.
149. The method of any one of embodiments 136-148, wherein the subject is eligible to receive a platinum-based chemotherapy regimen.
150. The method of any one of embodiments 136-149, wherein the first dosing cycle is initiated prior to a surgery.
151. The method of embodiment 150, wherein at least 1, 2, 3, or 4 dosing cycles are completed prior to the surgery.
152. The method of embodiment 150 or 151, wherein 4 dosing cycles are completed prior to the surgery.
153. The method of any one of embodiments 136-152, wherein at least one dosing cycle is initiated after a surgery.
154. The method of embodiment 153, wherein 16 dosing cycles are completed after the surgery.
155. The method of any one of embodiments 150-154, wherein the surgery is a segmentectomy, a lobectomy, a bilobectomy, or a pneumonectomy.
156. The method of any one of embodiments 136-155, wherein the method further comprises a radiotherapy.
157. The method of embodiment 156, wherein the radiotherapy is a post-operative radiotherapy.
158. The method of any one of embodiments 136-157, wherein the method further comprises administering one or more chemotherapeutic agents.
159. The method of embodiment 158, wherein the one or more chemotherapeutic agents are administered after a surgery.
160. The method of embodiment 159, wherein the one or more chemotherapeutic agents are administered in 4 dosing cycles after the surgery.
161. The method of any one of embodiments 158-160, wherein the one or more chemotherapeutic agents are a platinum-based chemotherapeutic agent and a non-platinum-based chemotherapeutic agent.
162. The method of embodiment 161, wherein:
(a) the platinum-based chemotherapeutic agent is carboplatin and the non-platinum-based chemotherapeutic agent is pemetrexed;
(b) the platinum-based chemotherapeutic agent is carboplatin and the non-platinum-based chemotherapeutic agent is gemcitabine;
(c) the platinum-based chemotherapeutic agent is carboplatin and the non-platinum-based chemotherapeutic agent is paclitaxel;
(d) the platinum-based chemotherapeutic agent is cisplatin and the non-platinum-based chemotherapeutic agent is pemetrexed; or
(e) the platinum-based chemotherapeutic agent is cisplatin and the non-platinum-based chemotherapeutic agent is gemcitabine.
163. The method of any one of embodiments 136-162, wherein the treating results in an increase in major pathological response (MPR) rate as compared to a reference MPR rate.
164. The method of embodiment 163, wherein the reference MPR rate is an MPR rate of population of subjects who have received a treatment comprising:
(a) a PD-1 axis binding antagonist without an anti-TIGIT antagonist antibody; and/or
(b) cisplatin and docetaxel or cisplatin, docetaxel, and bevacizumab.
165. The method of any one of embodiments 136-164, wherein the treating results in a pathological complete response (pCR) and/or an increase in pCR rate as compared to a reference pCR rate.
166. The method of embodiment 165, wherein the reference pCR rate is a pCR rate of population of subjects who have received a treatment comprising:
(a) a PD-1 axis binding antagonist without an anti-TIGIT antagonist antibody; and/or
(b) cisplatin and docetaxel or cisplatin, docetaxel, and bevacizumab.
167. The method of any one of embodiments 136-166, wherein the treating results in an increase in event-free survival (EFS) as compared to a reference EFS time.
168. The method of embodiment 167, wherein the reference EFS time is an EFS time of a population of subjects who have received a treatment comprising:
(a) a PD-1 axis binding antagonist without an anti-TIGIT antagonist antibody; and/or
(b) cisplatin and docetaxel or cisplatin, docetaxel, and bevacizumab.
169. A method for treating a subject having a resectable lung cancer, the method comprising administering to the subject one or more dosing cycles of an anti-TIGIT antagonist antibody at a dose of between about 30 mg to about 600 mg every three weeks and a PD-1 axis binding antagonist at a dose of between about 80 mg to about 1600 mg every three weeks.
170. A method for treating a subject having a lung cancer, the method comprising administering to the subject one or more dosing cycles of an anti-TIGIT antagonist antibody and a PD-1 axis binding antagonist, wherein at least one of the dosing cycles comprises administering to the subject the anti-TIGIT antagonist antibody at a dose of between about 30 mg to about 600 mg every three weeks and the PD-1 axis binding antagonist at a dose of between about 80 mg to about 1600 mg every three weeks as a neoadjuvant treatment.
171. A method for treating a subject having a resectable lung cancer, the method comprising administering to the subject one or more dosing cycles of an anti-TIGIT antagonist antibody at a dose of between about 30 mg to about 1200 mg every three weeks, a PD-1 axis binding antagonist at a dose of between about 80 mg to about 1600 mg every three weeks, a platinum-based chemotherapeutic agent, and a non-platinum-based chemotherapeutic agent.
172. A method for treating a subject having a lung cancer, the method comprising administering to the subject one or more dosing cycles of an anti-TIGIT antagonist antibody, a PD-1 axis binding antagonist, a platinum-based chemotherapeutic agent, and a non-platinum-based chemotherapeutic agent, wherein at least one of the dosing cycles comprises administering to the subject the anti-TIGIT antagonist antibody at a dose of between about 30 mg to about 1200 mg every three weeks, the PD-1 axis binding antagonist at a dose of between about 80 mg to about 1600 mg every three weeks, a platinum-based chemotherapeutic agent, and a non-platinum-based chemotherapeutic agent as a neoadjuvant treatment.
173. A method for treating a subject having a resectable lung cancer, the method comprising administering to the subject one or more dosing cycles of an anti-TIGIT antagonist antibody at a dose of between about 30 mg to about 1200 mg every three weeks, a PD-1 axis binding antagonist at a dose of between about 80 mg to about 1600 mg every three weeks, and: (a) (i) a platinum-based chemotherapeutic agent at a dose targeted to achieve an AUC of 5 mg/mL/min or an AUC of 6 mg/mL/min every three weeks; or (ii) a platinum-based chemotherapeutic agent at a dose of about 75 mg/m2 every three weeks; and (b) (i) an antimetabolite at a dose of about 500 mg/m2 every three weeks or about 1000 mg/m2 or about 1250 mg/m2 on Days 1 and 8 of each dosing cycle; or (ii) a taxane at a dose of about 100 mg/m2, about 175 mg/m2, or about 200 mg/m2 every three weeks.
174. A method for treating a subject having a lung cancer, the method comprising administering to the subject one or more dosing cycles of an anti-TIGIT antagonist antibody, a PD-1 axis binding antagonist, a platinum-based chemotherapeutic agent, and a non-platinum-based chemotherapeutic agent, wherein at least one of the dosing cycles comprises administering to the subject: (a) the anti-TIGIT antagonist antibody at a dose of between about 30 mg to about 600 mg every three weeks; (b) the PD-1 axis binding antagonist at a dose of between about 80 mg to about 1600 mg every three weeks; (c) the platinum-based chemotherapeutic agent: (i) at a dose targeted to achieve an AUC of 5 mg/mL/min or an AUC of 6 mg/mL/min every three weeks; or (ii) at a dose of about 75 mg/m2 every three weeks; and (d) the non-platinum-based chemotherapeutic agent, wherein the non-platinum-based chemotherapeutic agent is: (i) an antimetabolite at a dose of about 500 mg/m2 every three weeks or about 1000 mg/m2 or about 1250 mg/m2 on Days 1 and 8 of each dosing cycle; or (ii) a taxane at a dose of about 100 mg/m2, about 175 mg/m2, or about 200 mg/m2 every three weeks; wherein the treatment is a neoadjuvant treatment.
175. A method for treating a subject having a resectable lung cancer, the method comprising administering to the subject one or more dosing cycles of tiragolumab at a dose of about 600 mg every three weeks, atezolizumab at a dose of about 1200 mg every three weeks, and:
(a)
(i) carboplatin at a dose targeted to achieve an AUC of 5 mg/mL/min or an AUC of 6 mg/mL/min every three weeks; or
(ii) cisplatin at a dose of about 75 mg/m2 every three weeks; and
(b)
(i) pemetrexed at a dose of about 500 mg/m2 every three weeks or gemcitabine at a dose of about 1000 mg/m2 or about 1250 mg/m2 on Days 1 and 8 of each dosing cycle; or
(ii) paclitaxel at a dose of about 175 mg/m2 or about 200 mg/m2 every three weeks.
176. A method for treating a subject having a lung cancer, the method comprising administering to the subject one or more dosing cycles of tiragolumab, atezolizumab, a platinum-based chemotherapeutic agent, and a non-platinum-based chemotherapeutic agent, wherein at least one of the dosing cycles comprises administering to the subject: (a) tiragolumab at a dose of about 600 mg every three weeks; (b) atezolizumab at a dose of about 1200 mg every three weeks; (c) the platinum-based chemotherapeutic agent, wherein the platinum-based chemotherapeutic agent is: (i) carboplatin at a dose targeted to achieve an AUC of 5 mg/mL/min or an AUC of 6 mg/mL/min every three weeks; or (ii) cisplatin at a dose of about 75 mg/m2 every three weeks; and (d) the non-platinum-based chemotherapeutic agent, wherein the non-platinum-based chemotherapeutic agent is: (i) pemetrexed at a dose of about 500 mg/m2 every three weeks or gemcitabine at a dose of about 1000 mg/m2 or about 1250 mg/m2 on Days 1 and 8 of each dosing cycle; or (ii) paclitaxel at a dose of about 175 mg/m2 or about 200 mg/m2 every three weeks; wherein the treatment is a neoadjuvant treatment.
177. A method for treating a subject having a non-squamous NSCLC, the method comprising administering to the subject one or more dosing cycles of tiragolumab and atezolizumab, wherein: (a) at least one of the dosing cycles comprises administering to the subject tiragolumab at a dose of about 600 mg every three weeks and atezolizumab at a dose of 1200 mg every three weeks as a neoadjuvant treatment; and (b) at least one of the dosing cycles comprises administering to the subject tiragolumab at a dose of about 600 mg every three weeks and the atezolizumab at a dose of about 1200 mg every three weeks as an adjuvant treatment.
178. A method for treating a subject having a non-squamous NSCLC, the method comprising administering to the subject one or more dosing cycles of tiragolumab and atezolizumab, wherein: (I) at least one of the dosing cycles is a neoadjuvant treatment and comprises administering to the subject: (a) tiragolumab at a dose of about 600 mg every three weeks; (b) atezolizumab at a dose of about 1200 mg every three weeks as a neoadjuvant treatment; and (c) (i) carboplatin at a dose targeted to achieve an AUC of 5 mg/mL/min every three weeks and pemetrexed at a dose of about 500 mg/m2 every three weeks; (ii) carboplatin at a dose targeted to achieve an AUC of 6 mg/mL/min every three weeks and paclitaxel at a dose of about 175 mg/m2 or about 200 mg/m2 every three weeks; or (iii) cisplatin at a dose of about 75 mg/m2 every three weeks and pemetrexed at a dose of about 500 mg/m2 every three weeks; and (II) at least one of the dosing cycles comprises administering to the subject the anti-TIGIT antagonist antibody at a dose of between about 30 mg to about 1200 mg every three weeks and the PD-1 axis binding antagonist at a dose of between about 80 mg to about 1600 mg every three weeks as an adjuvant treatment.
179. A method for treating a subject having a lung cancer, the method comprising administering to the subject one or more dosing cycles of tiragolumab and atezolizumab, wherein: (I) at least one of the dosing cycles is a neoadjuvant treatment and comprises administering to the subject: (a) tiragolumab at a dose of about 600 mg every three weeks; (b) atezolizumab at a dose of about 1200 mg every three weeks as a neoadjuvant treatment; and (c) (i) carboplatin at a dose targeted to achieve an AUC of 5 mg/mL/min every three weeks and gemcitabine at a dose of about 1000 mg/m2 on Days 1 and 8 of each dosing cycle; (ii) carboplatin at a dose targeted to achieve an AUC of 6 mg/mL/min every three weeks and paclitaxel at a dose of about 175 mg/m2 or about 200 mg/m2 every three weeks; or (iii) cisplatin at a dose of about 75 mg/m2 every three weeks and gemcitabine at a dose of about 1250 mg/m2 on Days 1 and 8 of each dosing cycle; and (II) at least one of the dosing cycles comprises administering to the subject tiragolumab at a dose of between about 30 mg to about 1200 mg every three weeks and atezolizumab at a dose of between about 80 mg to about 1600 mg every three weeks as an adjuvant treatment.
180. A method for treating a subject having a lung cancer, the method comprising administering to the subject one or more dosing cycles of tiragolumab and atezolizumab, wherein:
(I) at least one of the dosing cycles is a neoadjuvant treatment and comprises administering to the subject:
(a) tiragolumab at a dose of about 1200 mg every three weeks;
(b) atezolizumab at a dose of about 1200 mg every three weeks as a neoadjuvant treatment; and
(c)
(i) carboplatin at a dose targeted to achieve an AUC of 5 mg/mL/min every three weeks and gemcitabine at a dose of about 1000 mg/m2 on Days 1 and 8 of each dosing cycle;
(ii) carboplatin at a dose targeted to achieve an AUC of 6 mg/mL/min every three weeks and paclitaxel at a dose of about 175 mg/m2 or about 200 mg/m2 every three weeks; or
(iii) cisplatin at a dose of about 75 mg/m2 every three weeks and gemcitabine at a dose of about 1250 mg/m2 on Days 1 and 8 of each dosing cycle; and
   (II) at least one of the dosing cycles comprises administering to the subject tiragolumab at a dose of between about 300 mg to about 800 mg every three weeks and atezolizumab at a dose of between about 900 mg to about 1500 mg every three weeks as an adjuvant treatment.
   181. A method for treating a subject having a lung cancer, the method comprising administering to the subject one or more dosing cycles of tiragolumab and atezolizumab, wherein: (I) at least one of the dosing cycles is a neoadjuvant treatment and comprises administering to the subject: (a) tiragolumab at a dose of about 600 mg every three weeks; (b) atezolizumab at a dose of about 1200 mg every three weeks as a neoadjuvant treatment; and (c) (i) carboplatin at a dose targeted to achieve an AUC of 5 mg/mL/min every three weeks and gemcitabine at a dose of about 1000 mg/m2 on Days 1 and 8 of each dosing cycle; (ii) carboplatin at a dose targeted to achieve an AUC of 6 mg/mL/min every three weeks and paclitaxel at a dose of about 175 mg/m2 or about 200 mg/m2 every three weeks; or (iii) cisplatin at a dose of about 75 mg/m2 every three weeks and gemcitabine at a dose of about 1250 mg/m2 on Days 1 and 8 of each dosing cycle; and (II) at least one of the dosing cycles comprises administering to the subject tiragolumab at a dose of about 600 mg every three weeks and atezolizumab at a dose of about 1200 mg every three weeks as an adjuvant treatment.
   182. A method for treating a subject having a resectable squamous NSCLC, the method comprising administering to the subject one or more dosing cycles of tiragolumab and atezolizumab, wherein: (I) at least one of the dosing cycles is a neoadjuvant treatment and comprises administering to the subject: (a) tiragolumab at a dose of about 600 mg every three weeks; (b) atezolizumab at a dose of about 1200 mg every three weeks as a neoadjuvant treatment; and (c) (i) carboplatin at a dose targeted to achieve an AUC of 5 mg/mL/min every three weeks and gemcitabine at a dose of about 1000 mg/m2 on Days 1 and 8 of each dosing cycle; (ii) carboplatin at a dose targeted to achieve an AUC of 6 mg/mL/min every three weeks and paclitaxel at a dose of about 175 mg/m2 or about 200 mg/m2 every three weeks; or (iii) cisplatin at a dose of about 75 mg/m2 every three weeks and gemcitabine at a dose of about 1250 mg/m2 on Days 1 and 8 of each dosing cycle; and (II) at least one of the dosing cycles comprises administering to the subject tiragolumab at a dose of about 600 mg every three weeks and atezolizumab at a dose of about 1200 mg every three weeks as an adjuvant treatment.
   183. The method of any one of embodiments 136-182, wherein a detectable protein expression level of PD-L1 determined by an IHC assay comprising staining with anti-PD-L1 antibody SP263 has been determined.
   184. The method of embodiment 183, wherein the detectable protein expression level of PD-L1 is a PD-L1-positive tumor cell fraction greater than, or equal to, 50%.
   185. The method of any one of embodiments 73, 114-121, and 136-184, wherein the lung cancer is an NSCLC.
   186. The method of embodiment 185, wherein the NSCLC is a squamous NSCLC.
   187. The method of embodiment 185, wherein the NSCLC is a non-squamous NSCLC.
   188. The method of embodiment 72, wherein the cancer is a cervical cancer.
   189. A method for treating a subject or population of subjects having a cervical cancer with a detectable expression level of PD-L1, the method comprising administering to the subject or a population of subjects one or more dosing cycles of an anti-TIGIT antagonist antibody at a dose of between about 30 mg to about 1200 mg every three weeks and a PD-1 axis binding antagonist at a dose of between about 80 mg to about 1600 mg every three weeks.
   190. A method for treating a subject or a population of subjects having a cervical cancer with a detectable expression level of PD-L1, the method comprising administering to the subject or population of subjects one or more dosing cycles of an anti-TIGIT antagonist antibody at a dose of between about 300 mg to about 800 mg every three weeks and a PD-1 axis binding antagonist at a dose of between about 900 mg to about 1500 mg every three weeks.
   191. A method of selecting a therapy for a subject having a cervical cancer, the method comprising:
   (a) detecting the protein expression level of PD-L1 on tumor cells from a tumor sample from the subject by an IHC assay using an anti-PD-L1 antibody suitable for staining; and
   (b) selecting for the subject having a detectable expression level of PD-L1 a therapy comprising one or more dosing cycles of an anti-TIGIT antagonist antibody administered at a dose of between about 30 mg to about 1200 mg every three weeks and a PD-1 axis binding antagonist administered at a dose of between about 80 mg to about 1600 mg every three weeks based on PD-L1 expression on tumor cells having been detected.
   192. The method any one of embodiments 189-191, wherein the cervical cancer is a squamous cell carcinoma, adenosquamous carcinoma, or adenocarcinoma.
   193. The method of any one of embodiments 189-191, wherein the cervical cancer is Stage IVB, metastatic, recurrent, or persistent.
   194. The method of any one of embodiments 189-193, wherein the cervical cancer is a metastatic and/or recurrent PD-L1-positive cervical carcinoma.
   195. The method of any one of embodiments 189-194, wherein the subject or subjects have received at least one line of prior therapy.
   196. The method of any one of embodiments 189-195, wherein the subject or subjects have received two lines of prior therapy.
   197. The method of any one of embodiments 189-196, wherein the subject or subjects have not received more than two lines of prior therapy.
   198. The method of any one of embodiments 189-194, wherein the subject or subjects have not received prior therapy.
   199. The method of any one of embodiments 195-198, wherein the prior therapy is chemotherapy, surgery, and/or radiotherapy.
   200. The method of any one of embodiments 189 and 192-199, wherein the treating results in a clinical response.
   201. The method of embodiment 200, wherein the clinical response is an increase in an objective response rate (ORR) of the population of subjects as compared to a reference ORR.
   202. The method of embodiment 201, wherein the reference ORR is the median ORR of a population of subjects who have received a treatment comprising a PD-1 axis binding antagonist without an anti-TIGIT antagonist antibody.
   203. The method of embodiment 200, wherein the reference ORR is at least about 14.6% to about 26%.
   204. The method of embodiment 200, wherein the clinical response is a CR or a PR.
   205. The method of any one of embodiments 200-204, wherein the clinical response is an increase in the progression-free survival (PFS) of the subject as compared to a reference PFS time, an increase in the duration of response (DOR) of the subject as compared to a reference DOR time, or an increase in the overall survival (OS) of the subject as compared to a reference OS time.
   206. The method of embodiment 205, wherein:
   (a) the reference PFS time is the median PFS time of a population of subjects who have received a treatment comprising a PD-1 axis binding antagonist without an anti-TIGIT antagonist antibody;
   (b) the reference DOR time is the median DOR time of a population of subjects who have received a treatment comprising a PD-1 axis binding antagonist without an anti-TIGIT antagonist antibody; or
   (c) the reference OS time is the median OS time of a population of subjects who have received a treatment comprising a PD-1 axis binding antagonist without an anti-TIGIT antagonist antibody.
   207. A method for treating a subject having a cervical cancer with a detectable expression level of PD-L1, the method comprising administering to the subject one or more dosing cycles of an anti-TIGIT antagonist antibody at a dose of 600 mg every three weeks and atezolizumab at a dose of 1200 mg every three weeks.
   208. A method of identifying a subject having a cervical cancer who is likely to benefit from a therapy comprising one or more dosing cycles of an anti-TIGIT antagonist antibody and atezolizumab, the method comprising:
   (a) providing a tumor sample from the subject;
   (b) detecting the protein expression level of PD-L1 in the tumor sample by an IHC assay using an anti-PD-L1 antibody suitable for staining; and
   (c) identifying the subject as one who is likely to benefit from a therapy comprising one or more dosing cycles of an anti-TIGIT antagonist antibody administered at a dose of 600 mg every three weeks and atezolizumab administered at a dose of 1200 mg every three weeks based on PD-L1 expression on tumor cells having been detected.
   209. A method of selecting a therapy for a subject having a cervical cancer, the method comprising:
   (a) detecting the protein expression level of PD-L1 on tumor cells from a tumor sample from the subject by an IHC assay using an anti-PD-L1 antibody suitable for staining; and
   (b) selecting for the subject having a detectable expression level of PD-L1 a therapy comprising one or more dosing cycles of an anti-TIGIT antagonist antibody administered at a dose of 600 mg every three weeks and atezolizumab administered at a dose of 1200 mg every three weeks based on PD-L1 expression on tumor cells having been detected.
   210. A method for treating a subject having a cervical cancer with a detectable expression level of PD-L1, the method comprising administering to the subject one or more dosing cycles of tiragolumab at a dose of about 600 mg every three weeks and atezolizumab at a dose of about 1200 mg every three weeks.
   211. A method of treating a subject having a cervical cancer, the method comprising:
   (a) providing a tumor sample from the subject;
   (b) detecting the protein expression level of PD-L1 in the tumor sample by an IHC assay using an anti-PD-L1 antibody suitable for staining;
   (c) identifying the subject as one who is likely to benefit from a therapy comprising one or more dosing cycles of tiragolumab administered at a dose of 600 mg every three weeks and atezolizumab administered at a dose of 1200 mg every three weeks based on PD-L1 expression on tumor cells having been detected; and
   (d) administering to the identified subject the therapy.
   212. A method of selecting a therapy for a subject having a cervical cancer, the method comprising:
   (a) detecting the protein expression level of PD-L1 on tumor cells from a tumor sample from the subject by an IHC assay using an anti-PD-L1 antibody suitable for staining; and
   (b) selecting for the subject having a detectable expression level of PD-L1 a therapy comprising one or more dosing cycles of tiragolumab administered at a dose of 600 mg every three weeks and atezolizumab administered at a dose of 1200 mg every three weeks based on PD-L1 expression on tumor cells having been detected.
   213. A method of treating a subject having a metastatic and/or recurrent cervical carcinoma, the method comprising:
   (a) providing a tumor sample from the subject;
   (b) detecting the protein expression level of PD-L1 in the tumor sample by an IHC assay using an anti-PD-L1 antibody suitable for staining;
   (c) identifying the subject as one who is likely to benefit from a therapy comprising one or more dosing cycles of tiragolumab administered at a dose of 600 mg every three weeks and atezolizumab administered at a dose of 1200 mg every three weeks based on PD-L1 expression on tumor cells having been detected; and
   (d) administering to the identified subject the therapy.
   214. A method of selecting a therapy for a subject having a metastatic and/or recurrent cervical carcinoma, the method comprising:
   (a) detecting the protein expression level of PD-L1 on tumor cells from a tumor sample from the subject by an IHC assay using an anti-PD-L1 antibody suitable for staining; and
   (b) selecting for the subject having a detectable expression level of PD-L1 a therapy comprising one or more dosing cycles of tiragolumab administered at a dose of 600 mg every three weeks and atezolizumab administered at a dose of 1200 mg every three weeks based on PD-L1 expression on tumor cells having been detected.
   215. The method of any one of embodiments 189-214, wherein the subject is identified as one who is likely to benefit from the treatment based on PD-L1 expression on tumor cells having been detected.
   216. The method of any one of embodiments 189-215, wherein PD-L1 expression level is detected using anti-PD-L1 antibody SP263.
   217. The method of any one of embodiments 189-216, wherein the detectable expression level of PD-L1 is a tumor-associated immune-cell (TIC) of greater than or equal to 5% in a sample from the subject.
   218. The method of any one of embodiments 189-215, wherein PD-L1 expression level is detected using anti-PD-L1 antibody 22C3.
   219. The method of any one of embodiments 189-216, wherein the detectable expression level of PD-L1 is a combined positive score (CPS) of greater than or equal to 1 in a sample from the subject.
   220. The method of embodiment 72, wherein the cancer is a breast cancer.
   221. A method of treating a subject or population of subjects having a breast cancer, the method comprising administering to the subject or population of subjects a dosing regimen comprising one or more dosing cycles of tiragolumab at a dose of about 840 mg every four weeks, atezolizumab at a dose of about 1680 mg every four weeks, and nab-paclitaxel at a dose of about 100 mg/m2 for 3-weeks on/1-week off.
   222. The method of embodiment 220 or 221, wherein the breast cancer is a triple-negative breast cancer (TNBC).
   223. The method of embodiment 222, wherein the TNBC is an unresectable locally advanced or metastatic TNBC.
   224. The method of any one of embodiments 220-223, wherein the subject has not received prior systemic therapy for metastatic breast cancer.
   225. The method of any one of embodiments 220-224, wherein the treatment results in an ORR of the population of subjects of at least about 53% to about 67.5%.
   226. The method of any one of embodiments 222-225, wherein the treatment results in a median OS of the population of subjects of about 25.0 months.
   227. A method of treating a subject having an early triple-negative breast cancer (eTNBC), the method comprising administering to the subject a dosing regimen comprising one or more dosing cycles of an anti-TIGIT antagonist antibody at a dose of about 10 mg to about 1000 mg every two weeks and a PD-1 axis binding antagonist at a dose of about 20 mg to about 1600 mg every two weeks.
   228. The method of embodiment 227, wherein the method comprises further administering to the subject one or more chemotherapeutic agents.
   229. The method of embodiment 228, wherein the one or more chemotherapeutic agents are a platinum-based chemotherapeutic agent, a taxane, a topoisomerase II inhibitor, or an alkylating agent.
   230. The method of embodiment 227, wherein the method further comprises administering (a) one or more dosing cycles of the anti-TIGIT antagonist antibody, the PD-1 axis binding antagonist, and a taxane or a taxane and a platinum-based chemotherapeutic agent; and (b) one or more dosing cycles of the anti-TIGIT antagonist antibody, the PD-1 axis binding antagonist, a topoisomerase II inhibitor, an alkylating agent, and granulocyte colony-stimulating factor (G-CSF) or granulocyte-macrophage colony-stimulating factor (GM-CSF).
   231. The method of embodiment 229 or 230, wherein the alkylating agent is cyclophosphamide.
   232. The method of embodiment 231, wherein the cyclophosphamide is administered at a dose of about 600 mg/m2.
   233. The method of any one of embodiments 227-229, 231, and 232, wherein the method comprises further administering to the subject a G-CSF, or GM-CSF.
   234. The method of embodiment 230 or 233, wherein the G-CSF is pegfilgrastim or filgrastim.
   235. The method of embodiment 234, wherein the G-CSF is pegfilgrastim.
   236. The method of embodiment 235, wherein the pegfilgrastim is administered at a dose of about 6 mg.
   237. The method of any one of embodiments 228, 229, and 231-236, wherein the method comprises further administering to the subject one or more subsequent doses of the one or more chemotherapeutic agents and/or G-CSF or GM-CSF.
   238. The method of any one of embodiments 228, 229, and 231-237, wherein the one or more chemotherapeutic agents and/or G-CSF or GM-CSF are each administered once per week, once every two weeks, or once every three weeks.
   239. The method of any one of embodiments 229-238, wherein the platinum-based chemotherapeutic agent is administered every three weeks, the taxane is administered every week, the topoisomerase II inhibitor is administered every two weeks, the alkylating agent is administered every two weeks, and the G-CSF or GM-CSF is administered every two weeks.
   240. The method of any one of embodiments 229-239, wherein the taxane or the taxane and the platinum-based chemotherapeutic agent are administered for the first 12 weeks of the dosing regimen.
   241. The method of any one of embodiments 229-240, wherein the topoisomerase II inhibitor, the alkylating agent, and the G-CSF or GM-CSF are administered during weeks 13-19 of the dosing regimen.
   242. The method of any one of embodiments 227-241, wherein the total length of the dosing regimen is 19 weeks.
   243. The method of any one of embodiments 227-242, wherein the method is a neoadjuvant treatment.
   244. The method of any one of embodiments 227-243, wherein the dosing regimen is followed by surgery.
   245. The method of embodiment 244, wherein the surgery is performed between two and six weeks after the last dose of the dosing regimen.
   246. The method of embodiment 244 or 245, wherein the surgery comprises a mastectomy.
   247. The method of any one of embodiments 244-246, wherein the surgery comprises an axillary lymph node surgery.
   248. The method of any one of embodiments 229-247, wherein the topoisomerase II inhibitor is doxorubicin.
   249. The method of any one of embodiments 229-247, wherein the taxane is nab-paclitaxel, the platinum-based chemotherapeutic agent is carboplatin, and the topoisomerase II inhibitor is doxorubicin.
   250. The method of embodiment 248 or 249, wherein doxorubicin is administered at a dose of about 60 mg/m2.
   251. A method of treating a subject having an eTNBC, the method comprising administering to the subject a dosing regimen comprising an anti-TIGIT antagonist antibody at a dose of about 300 mg to about 600 mg every two weeks, a PD-1 axis binding antagonist at a dose of about 600 mg to about 1200 mg every two weeks, and:
   (a)
   (i) a taxane at a dose of about 125 mg/m2 every week and a platinum agent at a dose targeted to achieve an AUC of 5 mg/mL/min every three weeks for the first 12 weeks of the dosing regimen; and
   (ii) a topoisomerase II inhibitor at a dose of about 60 mg/m2 every two weeks, an alkylating agent at a dose of about 600 mg/m2 every two weeks, and G-CSF or GM-CSF every two weeks for weeks 13-19 of the dosing regimen; or
   (b)
   (i) a taxane at a dose of about 125 mg/m2 every week for the first 12 weeks of the dosing regimen; and
   (ii) a topoisomerase II inhibitor at a dose of about 60 mg/m2 every two weeks, an alkylating agent at a dose of about 600 mg/m2 every two weeks, and G-CSF or GM-CSF every two weeks for weeks 13-19 of the dosing regimen;
   wherein the method further comprises surgery between two and six weeks after the last dose of the dosing regimen.
   252. A method of treating a subject having an eTNBC, the method comprising administering to the subject a dosing regimen comprising an anti-TIGIT antagonist antibody at a dose of about 300 mg to about 600 mg every two weeks, a PD-L1 binding antagonist at a dose of about 600 mg to about 1200 mg every two weeks, and:
   (a)
   (i) a taxane at a dose of about 125 mg/m2 every week and a platinum agent at a dose targeted to achieve an AUC of 5 mg/mL/min every three weeks for the first 12 weeks of the dosing regimen; and
   (ii) a topoisomerase II inhibitor at a dose of about 60 mg/m2 every two weeks, an alkylating agent at a dose of about 600 mg/m2 every two weeks, and G-CSF or GM-CSF every two weeks for weeks 13-19 of the dosing regimen; or
   (b)
   (i) a taxane at a dose of about 125 mg/m2 every week for the first 12 weeks of the dosing regimen; and
   (ii) a topoisomerase II inhibitor at a dose of about 60 mg/m2 every two weeks, an alkylating agent at a dose of about 600 mg/m2 every two weeks, and G-CSF or GM-CSF every two weeks for weeks 13-19 of the dosing regimen;
   wherein the method further comprises surgery between two and six weeks after the last dose of the dosing regimen.
   253. A method of treating a subject having an eTNBC, the method comprising administering to the subject a dosing regimen comprising an anti-TIGIT antagonist antibody at a dose of about 300 mg to about 600 mg every two weeks, an anti-PD-L1 antagonist antibody at a dose of about 600 mg to about 1200 mg every two weeks, and:
   (a)
   (i) a taxane at a dose of about 125 mg/m2 every week and a platinum agent at a dose targeted to achieve an AUC of 5 mg/mL/min every three weeks for the first 12 weeks of the dosing regimen; and
   (ii) a topoisomerase II inhibitor at a dose of about 60 mg/m2 every two weeks, an alkylating agent at a dose of about 600 mg/m2 every two weeks, and G-CSF or GM-CSF every two weeks for weeks 13-19 of the dosing regimen; or
   (b)
   (i) a taxane at a dose of about 125 mg/m2 every week for the first 12 weeks of the dosing regimen; and
   (ii) a topoisomerase II inhibitor at a dose of about 60 mg/m2 every two weeks, an alkylating agent at a dose of about 600 mg/m2 every two weeks, and G-CSF or GM-CSF every two weeks for weeks 13-19 of the dosing regimen;
   wherein the method further comprises surgery between two and six weeks after the last dose of the dosing regimen.
   254. A method of treating a subject having an eTNBC, the method comprising administering to the subject a dosing regimen comprising tiragolumab at a dose of about 300 mg to about 600 mg every two weeks, an anti-PD-L1 antagonist antibody at a dose of about 600 mg to about 1200 mg every two weeks, and:
   (a)
   (i) a taxane at a dose of about 125 mg/m2 every week and a platinum agent at a dose targeted to achieve an AUC of 5 mg/mL/min every three weeks for the first 12 weeks of the dosing regimen; and
   (ii) a topoisomerase II inhibitor at a dose of about 60 mg/m2 every two weeks, an alkylating agent at a dose of about 600 mg/m2 every two weeks, and G-CSF or GM-CSF every two weeks for weeks 13-19 of the dosing regimen; or
   (b)
   (i) a taxane at a dose of about 125 mg/m2 every week for the first 12 weeks of the dosing regimen; and
   (ii) a topoisomerase II inhibitor at a dose of about 60 mg/m2 every two weeks, an alkylating agent at a dose of about 600 mg/m2 every two weeks, and G-CSF or GM-CSF every two weeks for weeks 13-19 of the dosing regimen;
   wherein the method further comprises surgery between two and six weeks after the last dose of the dosing regimen.
   255. A method of treating a subject having an eTNBC, the method comprising administering to the subject a dosing regimen comprising an anti-TIGIT antagonist antibody at a dose of about 300 mg to about 600 mg every two weeks, atezolizumab at a dose of about 600 mg to about 1200 mg every two weeks, and:
   (a)
   (i) a taxane at a dose of about 125 mg/m2 every week and a platinum agent at a dose targeted to achieve an AUC of 5 mg/mL/min every three weeks for the first 12 weeks of the dosing regimen; and
   (ii) a topoisomerase II inhibitor at a dose of about 60 mg/m2 every two weeks, an alkylating agent at a dose of about 600 mg/m2 every two weeks, and G-CSF or GM-CSF every two weeks for weeks 13-19 of the dosing regimen; or
   (b)
   (i) a taxane at a dose of about 125 mg/m2 every week for the first 12 weeks of the dosing regimen; and
   (ii) a topoisomerase II inhibitor at a dose of about 60 mg/m2 every two weeks, an alkylating agent at a dose of about 600 mg/m2 every two weeks, and G-CSF or GM-CSF every two weeks for weeks 13-19 of the dosing regimen;
   wherein the method further comprises surgery between two and six weeks after the last dose of the dosing regimen.
   256. A method of treating a subject having an eTNBC, the method comprising administering to the subject a dosing regimen comprising tiragolumab at a dose of about 300 mg to about 600 mg every two weeks, atezolizumab at a dose of about 600 mg to about 1200 mg every two weeks, and:
   (a)
   (i) nab-paclitaxel at a dose of about 125 mg/m2 every week and carboplatin at a dose targeted to achieve an AUC of 5 mg/mL/min every three weeks for the first 12 weeks of the dosing regimen; and
   (ii) doxorubicin at a dose of about 60 mg/m2 every two weeks, cyclophosphamide at a dose of about 600 mg/m2 every two weeks, and G-CSF or GM-CSF every two weeks for weeks 13-19 of the dosing regimen; or
   (b)
   (i) nab-paclitaxel at a dose of about 125 mg/m2 every week for the first 12 weeks of the dosing regimen; and
   (ii) doxorubicin at a dose of about 60 mg/m2 every two weeks, cyclophosphamide at a dose of about 600 mg/m2 every two weeks, and G-CSF or GM-CSF every two weeks for weeks 13-19 of the dosing regimen;
   wherein the method further comprises surgery between two and six weeks after the last dose of the dosing regimen.
   257. A method of treating a subject having an eTNBC, the method comprising administering to the subject a dosing regimen comprising tiragolumab at a dose of about 420 mg every two weeks, atezolizumab at a dose of about 840 mg every two weeks, and:
   (a)
   (i) nab-paclitaxel at a dose of about 125 mg/m2 every week and carboplatin at a dose targeted to achieve an AUC of 5 mg/mL/min every three weeks for the first 12 weeks of the dosing regimen; and
   (ii) doxorubicin at a dose of about 60 mg/m2 every two weeks, cyclophosphamide at a dose of about 600 mg/m2 every two weeks, and G-CSF or GM-CSF every two weeks for weeks 13-19 of the dosing regimen; or
   (b)
   (i) nab-paclitaxel at a dose of about 125 mg/m2 every week for the first 12 weeks of the dosing regimen; and
   (ii) doxorubicin at a dose of about 60 mg/m2 every two weeks, cyclophosphamide at a dose of about 600 mg/m2 every two weeks, and G-CSF or GM-CSF every two weeks for weeks 13-19 of the dosing regimen;
   wherein the method further comprises surgery between two and six weeks after the last dose of the dosing regimen.
   258. The method of any one of embodiments 220-257, wherein a detectable protein expression level of PD-L1 in a tumor sample from the subject is determined by an IHC assay comprising staining with anti-PD-L1 antibody SP142 has been determined.
   259. The method of embodiment 258, wherein a proportion of tumor area occupied by PD-L1 expressing tumor-infiltrating immune cells (ICs) in the tumor sample is greater than or equal to 1%.
   260. The method of any one of embodiments 227-259, wherein the eTNBC is a T2-4d TNBC at presentation.
   261. The method of any one of embodiments 227-260, wherein the eTNBC is a cT2-cT4, cN0-cN3, and cM0 TNBC at presentation.
   262. The method of any one of embodiments 227-261, wherein the subject has not been previously treated for eTNBC.
   263. The method of any one of embodiments 227-262, wherein the treatment results in a pathological complete response (pCR).
   264. The method of any one of embodiments 227-263, wherein the treatment results in an increase in overall survival (OS) or event-free survival (EFS).
   265. The method of embodiment 72, wherein the cancer is a head and neck cancer.
   266. The method of embodiment 265, wherein the head and neck cancer is a squamous cell carcinoma of the head and neck (SCCHN).
   267. A method for treating a subject or population of subjects having an SCCHN with a detectable expression level of PD-L1, the method comprising administering to the subject or population of subjects one or more dosing cycles of an anti-TIGIT antagonist antibody at a dose of between about 30 mg to about 1200 mg every three weeks and a PD-1 axis binding antagonist at a dose of between about 80 mg to about 1600 mg every three weeks.
   268. A method for treating a subject or population of subjects having an SCCHN with a detectable expression level of PD-L1, the method comprising administering to the subject or population of subjects one or more dosing cycles of an anti-TIGIT antagonist antibody at a dose of between about 300 mg to about 800 mg every three weeks and a PD-1 axis binding antagonist at a dose of between about 900 mg to about 1500 mg every three weeks.
   269. A method of selecting a therapy for a subject or a population of subjects having an SCCHN, the method comprising:
   (a) detecting a protein expression level of PD-L1 in a tumor sample from the subject by an IHC assay using an anti-PD-L1 antibody suitable for staining; and
   (b) selecting for the subject or a population of subjects having a detectable expression level of PD-L1 a therapy comprising one or more dosing cycles of a PD-1 axis binding antagonist at a dose of between about 80 mg to about 1600 mg every three weeks and an anti-TIGIT antagonist antibody at a dose of between about 30 mg to about 1200 mg every three weeks based on PD-L1 expression having been detected.
   270. A method for treating a subject or population of subjects having an SCCHN with a detectable expression level of PD-L1, the method comprising administering to the subject or population of subjects one or more dosing cycles of tiragolumab at a dose of about 600 mg every three weeks and atezolizumab at a dose of about 1200 mg every three weeks.
   271. The method of any one of embodiments 267-270, wherein a tumor sample obtained from the subject or subjects have been determined to have a detectable expression level of PD-L1.
   272. The method of any one of embodiments 266-271, wherein the SSCHN is human papillomavirus (HPV)-positive.
   273. The method of any one of embodiments 266-272, wherein the SSCHN is HPV-negative.
   274. The method of embodiment 272 or 273, wherein HPV status is determined by p16 IHC, in situ hybridization, or by PCR.
   275. The method of any one of embodiments 266-274, wherein the SCCHN is a recurrent and/or metastatic SCCHN.
   276. The method of any one of embodiments 265-275, wherein the subject or subjects have not received prior therapy.
   277. The method of embodiment 276, wherein the prior therapy is a prior systemic therapy for recurrent and/or metastatic disease.
   278. The method of any one of embodiments 265-268 and 270-277, wherein the treating results in a CR or PR.
   279. The method of any one of embodiments 265-268 and 270-277, wherein the treating results in an increase in the objective response rate (ORR) of the population of subjects as compared to a reference ORR.
   280. The method of embodiment 279, wherein the reference ORR is the median ORR of a population of subjects who have received a treatment comprising a PD-1 axis binding antagonist without an anti-TIGIT antagonist antibody.
   281. The method of embodiment 279 or 280, wherein the reference ORR is about 19% to about 36%.
   282. The method of any one of embodiments 265-268 and 270-281, wherein the treating results in an increase in the progression-free survival (PFS) of the subject or population of subjects as compared to a reference PFS time, an increase in the duration of response (DOR) of the subject or population of subjects as compared to a reference DOR time, or an increase in the overall survival (OS) of the subject or population of subjects as compared to a reference OS time.
   283. The method of embodiment 282, wherein:
   (a) the reference PFS time is the median PFS time of a population of subjects who have received a treatment comprising a PD-1 axis binding antagonist without an anti-TIGIT antagonist antibody;
   (b) the reference DOR time is the median DOR time of a population of subjects who have received a treatment comprising a PD-1 axis binding antagonist without an anti-TIGIT antagonist antibody; or
   (c) the reference OS time is the median OS time of a population of subjects who have received a treatment comprising a PD-1 axis binding antagonist without an anti-TIGIT antagonist antibody.
   284. The method of embodiment 282, wherein the reference DOR time is at least about 6.7 months to about 23.4 months.
   285. The method of embodiment 282, wherein the reference OS time is at least about 11.6 months to about 14.9 months.
   286. A method for treating a subject having an SCCHN with a detectable expression level of PD-L1, the method comprising administering to the subject one or more dosing cycles of a PD-1 axis binding antagonist at a dose of between about 80 mg to about 1600 mg every three weeks and an anti-TIGIT antagonist antibody at a dose of between about 30 mg to about 1200 mg every three weeks.
   287. A method for treating a subject having an SCCHN with a detectable expression level of PD-L1, the method comprising administering to the subject one or more dosing cycles of a PD-1 axis binding antagonist at a dose of about 1200 mg every three weeks and an anti-TIGIT antagonist antibody at a dose of about 600 mg every three weeks.
   288. A method for treating a subject having an SCCHN with a detectable expression level of PD-L1, the method comprising administering to the subject one or more dosing cycles of a PD-L1 binding antagonist at a dose of between about 80 mg to about 1600 mg every three weeks and an anti-TIGIT antagonist antibody at a dose of between about 30 mg to about 1200 mg every three weeks.
   289. A method for treating a subject having an SCCHN with a detectable expression level of PD-L1, the method comprising administering to the subject one or more dosing cycles of atezolizumab at a dose of between about 80 mg to about 1600 mg every three weeks and tiragolumab at a dose of between about 30 mg to about 1200 mg every three weeks.
   290. A method for treating a subject having an SCCHN with a detectable expression level of PD-L1, the method comprising administering to the subject one or more dosing cycles of tiragolumab at a dose of about 600 mg every three weeks and atezolizumab at a dose of about 1200 mg every three weeks.
   291. A method of treating a subject having an SCCHN, the method comprising:
   (a) providing a tumor sample from the subject;
   (b) detecting a protein expression level of PD-L1 in the tumor sample by an IHC assay using an anti-PD-L1 antibody suitable for staining;
   (c) identifying the subject as one who is likely to benefit from a therapy comprising one or more dosing cycles of a PD-1 axis binding antagonist at a dose of between about 80 mg to about 1600 mg every three weeks and an anti-TIGIT antagonist antibody at a dose of between about 30 mg to about 1200 mg every three weeks; and
   (d) administering to the identified subject having a detectable expression level of PD-L1 the therapy.
   292. A method of treating a subject having an SCCHN, the method comprising:
   (a) providing a tumor sample from the subject;
   (b) detecting a protein expression level of PD-L1 in the tumor sample by an IHC assay using an anti-PD-L1 antibody suitable for staining;
   (c) identifying the subject as one who is likely to benefit from a therapy comprising one or more dosing cycles of a PD-1 axis binding antagonist at a dose of about 1200 mg every three weeks and an anti-TIGIT antagonist antibody at a dose of about 600 mg every three weeks; and
   (d) administering to the identified subject having a detectable expression level of PD-L1 the therapy.
   293. A method of treating a subject having an SCCHN, the method comprising:
   (a) providing a tumor sample from the subject;
   (b) detecting a protein expression level of PD-L1 in the tumor sample by an IHC assay using an anti-PD-L1 antibody suitable for staining;
   (c) identifying the subject as one who is likely to benefit from a therapy comprising one or more dosing cycles of atezolizumab at a dose of between about 80 mg to about 1600 mg every three weeks and tiragolumab at a dose of between about 30 mg to about 1200 mg every three weeks; and
   (d) administering to the identified subject having a detectable expression level of PD-L1 the therapy.
   294. A method of treating a subject having an SCCHN, the method comprising:
   (a) providing a tumor sample from the subject;
   (b) detecting a protein expression level of PD-L1 in the tumor sample by an IHC assay using an anti-PD-L1 antibody suitable for staining;
   (c) identifying the subject as one who is likely to benefit from a therapy comprising one or more dosing cycles of atezolizumab at a dose of about 1200 mg every three weeks and tiragolumab at a dose of about 600 mg every three weeks; and
   (d) administering to the identified subject having a detectable expression level of PD-L1 the therapy.
   295. A method of selecting a therapy for a subject having an SCCHN, the method comprising:
   (a) detecting a protein expression level of PD-L1 in a tumor sample from the subject by an IHC assay using an anti-PD-L1 antibody suitable for staining; and
   (b) selecting for the subject having a detectable expression level of PD-L1 a therapy comprising one or more dosing cycles of a PD-1 axis binding antagonist at a dose of between about 80 mg to about 1600 mg every three weeks and an anti-TIGIT antagonist antibody at a dose of between about 30 mg to about 1200 mg every three weeks based on PD-L1 expression having been detected.
   296. A method of selecting a therapy for a subject having an SCCHN, the method comprising:
   (a) detecting a protein expression level of PD-L1 in a tumor sample from the subject by an IHC assay using an anti-PD-L1 antibody suitable for staining; and
   (b) selecting for the subject having a detectable expression level of PD-L1 a therapy comprising one or more dosing cycles of a PD-1 axis binding antagonist at a dose of about 1200 mg every three weeks and an anti-TIGIT antagonist antibody at a dose of about 600 mg every three weeks based on PD-L1 expression having been detected.
   297. A method of selecting a therapy for a subject having an SCCHN, the method comprising:
   (a) detecting a protein expression level of PD-L1 in a tumor sample from the subject by an IHC assay using an anti-PD-L1 antibody suitable for staining; and
   (b) selecting for the subject having a detectable expression level of PD-L1 a therapy comprising one or more dosing cycles of atezolizumab at a dose of between about 80 mg to about 1600 mg every three weeks and tiragolumab at a dose of between about 30 mg to about 1200 mg every three weeks based on PD-L1 expression having been detected.
   298. A method of selecting a therapy for a subject having an SCCHN, the method comprising:
   (a) detecting a protein expression level of PD-L1 in a tumor sample from the subject by an IHC assay using an anti-PD-L1 antibody suitable for staining; and
   (b) selecting for the subject having a detectable expression level of PD-L1 a therapy comprising one or more dosing cycles of atezolizumab at a dose of about 1200 mg every three weeks and tiragolumab at a dose of about 600 mg every three weeks based on PD-L1 expression having been detected.
   299. The method of any one of embodiments 267-298, wherein the detectable expression level of PD-L1 is a detectable protein expression level of PD-L1 determined by an immunohistochemical (IHC) assay comprising staining with anti-PD-L1 antibody SP263.
   300. The method of embodiment 299, wherein the detectable protein expression level of PD-L1 is a tumor-associated immune-cell (TIC) of:
   (a) greater than or equal to 5%;
   (b) greater than or equal to 5% and less than 20%; or
   (c) greater than or equal to 20% in the tumor sample.
   301. A method of identifying a subject having an SCCHN as one who is likely to benefit from a therapy comprising one or more dosing cycles of a PD-1 axis binding antagonist at a dose of between about 80 mg to about 1600 mg every three weeks and an anti-TIGIT antagonist antibody at a dose of between about 30 mg to about 1200 mg every three weeks, the method comprising determining a protein expression level of PD-L1 in a tumor sample from the subject by an IHC assay using an anti-PD-L1 antibody suitable for staining, wherein a protein expression level of PD-L1 of greater than or equal to a TIC of 5% identifies the subject as likely to benefit from the therapy.
   302. A method of identifying a subject having an SCCHN as one who is likely to benefit from a therapy comprising one or more dosing cycles of a PD-1 axis binding antagonist at a dose of between about 80 mg to about 1600 mg every three weeks and an anti-TIGIT antagonist antibody at a dose of between about 30 mg to about 1200 mg every three weeks, the method comprising determining a protein expression level of PD-L1 in a tumor sample from the subject by an IHC assay using an anti-PD-L1 antibody suitable for staining, wherein a protein expression level of PD-L1 of greater than or equal to a TIC of 5% and less than 20% identifies the subject as likely to benefit from the therapy.
   303. A method of identifying a subject having an SCCHN as one who is likely to benefit from a therapy comprising one or more dosing cycles of a PD-1 axis binding antagonist at a dose of between about 80 mg to about 1600 mg every three weeks and an anti-TIGIT antagonist antibody at a dose of between about 30 mg to about 1200 mg every three weeks, the method comprising determining a protein expression level of PD-L1 in a tumor sample from the subject by an IHC assay using an anti-PD-L1 antibody suitable for staining, wherein a protein expression level of PD-L1 of greater than or equal to a TIC of 20% identifies the subject as likely to benefit from the therapy.
   304. The method of any one of embodiments 301-303, wherein the subject is identified as likely to benefit from the therapy and the method further comprises administering to the subject the therapy.
   305. The method of any one of embodiments 301-304, wherein the TIC is determined using a Ventana SP263 IHC assay.
   306. The method of any one of embodiments 267-305, wherein the subject or population of subjects are identified as one who is likely to benefit from the treatment based on PD-L1 expression on tumor cells having been detected.
   307. The method of embodiment 72, wherein the cancer is a liver cancer.
   308. The method of embodiment 307, wherein the liver cancer is a hepatocellular carcinoma (HCC).
   309. A method of treating a subject or population of subjects having a hepatocellular carcinoma (HCC), the method comprising administering to the subject or population of subjects one or more dosing cycles of an anti-TIGIT antagonist antibody and a PD-1 axis binding antagonist, wherein the subject or population of subjects have received no prior systemic treatment for HCC.
   310. The method of embodiment 309, wherein the method further comprises administering to the subject or population of subjects a VEGF antagonist.
   311. A method of treating a subject or population of subjects having an HCC, the method comprising administering to the subject or population of subjects one or more dosing cycles of an anti-TIGIT antagonist antibody, a PD-1 axis binding antagonist, and a VEGF antagonist.
   312. The method of embodiment 310 and 311, wherein the VEGF antagonist is administered at a dose of about 5 mg/kg to about 25 mg/kg every three weeks.
   313. The method of any one of embodiments 309-312, wherein the anti-TIGIT antagonist antibody is administered at a dose of about 30 mg to about 1200 mg every three weeks.
   314. The method of embodiment 313, wherein the anti-TIGIT antagonist antibody is administered at a dose of about 30 mg to about 800 mg every three weeks.
   315. The method of any one of embodiments 309-314, wherein the anti-TIGIT antagonist antibody is administered at a dose of about 600 mg every three weeks.
   316. The method of any one of embodiments 309-315, wherein the PD-1 axis binding antagonist is administered at a dose of about 80 mg to about 1600 mg every three weeks.
   317. The method of any one of embodiments 309-316, wherein the PD-1 axis binding antagonist is administered at a dose of about 900 mg to about 1500 mg every three weeks.
   318. The method of embodiment 310 or 311, wherein the VEGF antagonist is administered at a dose of about 1 mg/kg to about 20 mg/kg every two weeks.
   319. The method of embodiment 318, wherein the VEGF antagonist is administered at a dose of about 5 mg/kg to about 10 mg/kg every two weeks.
   320. The method of embodiment 319, wherein the VEGF antagonist is administered at a dose of about 5 mg/kg, about 7.5 mg/kg, or about 10 mg/kg every two weeks.
   321. The method of any one of embodiments 309-311 and 318-320, wherein the anti-TIGIT antagonist antibody is administered at a dose of about 10 mg to about 1000 mg every two weeks.
   322. The method of any one of embodiments 309-311 and 318-321, wherein the PD-1 axis binding antagonist is administered at a dose of about 20 mg to about 1600 mg every two weeks.
   323. The method of embodiment 309-311, wherein the anti-TIGIT antagonist antibody is administered at a dose of about 200 mg to about 2000 mg every four weeks.
   324. The method of any one of embodiments 309-311 and 323, wherein the PD-1 axis binding antagonist is administered at a dose of about 80 mg to about 2000 mg every four weeks.
   325. The method of any one of embodiments 309-311, 323, and 324, wherein the PD-1 axis binding antagonist is administered at a dose of about 1400 mg to about 2000 mg every four weeks.
   326. The method of any one of embodiments 309-325, wherein the HCC is a locally advanced or metastatic HCC.
   327. The method of any one of embodiments 309-326, wherein the HCC is an unresectable HCC.
   328. The method of any one of embodiments 309-327, wherein the subject or subjects have been determined to have adequate liver function.
   329. The method of embodiment 328, wherein the adequate liver function is characterized as Child-Pugh class A.
   330. The method of any one of embodiments 309-329, wherein an HCC tumor sample obtained from the subject or subjects have been determined to have a detectable expression level of PD-L1.
   331. The method of any one of embodiments 309-330, wherein the method comprises administering to the subject or population of subjects at least four dosing cycles.
   332. The method of embodiment 331, wherein the method comprises administering to the subject or population of subjects at least 16 dosing cycles.
   333. A method of treating a subject or population of subjects having an HCC, the method comprising administering to the subject one or more dosing cycles of tiragolumab at a dose of about 600 mg every three weeks, atezolizumab at a dose of about 1200 mg every three weeks, and bevacizumab at a dose of about 15 mg/kg every three weeks.
   334. The method of any one of embodiments 311-333, wherein the subject or subjects have received no prior systemic treatment for HCC.
   335. The method of any one of embodiments 309-334, wherein the treatment results in a median PFS of the population of subjects of at least about 5.6 months to at least about 6.83 months.
   336. The method of embodiment 72, wherein the cancer is a bladder cancer.
   337. The method of embodiment 336, wherein the bladder cancer is a muscle-invasive bladder cancer (MIBC).
   338. A method for treating a subject or population of subjects having an MIBC, the method comprising administering to the subject or population of subjects one or more dosing cycles of an anti-TIGIT antagonist antibody at a dose of between about 30 mg to about 1200 mg every three weeks and a PD-1 axis binding antagonist at a dose of between about 80 mg to about 1600 mg every three weeks, wherein the subject is ineligible for treatment with a platinum-based chemotherapeutic agent.
   339. A method for treating a subject or population of subjects having an MIBC, the method comprising administering to the subject or population of subjects one or more dosing cycles of an anti-TIGIT antagonist antibody at a dose of between about 300 mg to about 800 mg every three weeks and a PD-1 axis binding antagonist at a dose of between about 900 mg to about 1500 mg every three weeks, wherein the subject is ineligible for treatment with a platinum-based chemotherapeutic agent.
   340. A method for treating a subject or population of subjects having an MIBC, the method comprising administering to the subject or population of subjects one or more dosing cycles of an anti-TIGIT antagonist antibody at a dose of between about 30 mg to about 1200 mg every three weeks and a PD-1 axis binding antagonist at a dose of between about 80 mg to about 1600 mg every three weeks, wherein the treatment is a perioperative treatment.
   341. A method for treating a subject or population of subjects having an MIBC, the method comprising administering to the subject or population of subjects one or more dosing cycles of an anti-TIGIT antagonist antibody at a dose of between about 300 mg to about 800 mg every three weeks and a PD-1 axis binding antagonist at a dose of between about 900 mg to about 1500 mg every three weeks, wherein the treatment is a perioperative treatment.
   342. The method of any one of embodiments 338-341, wherein the subject or subjects have a creatinine clearance of <60 mL/min.
   343. The method of any one of embodiments 338-342, wherein the subject or subjects have a greater than or equal to grade 2 hearing loss.
   344. The method of any one of embodiments 338-343, wherein the subject or subjects have a greater than or equal to grade 2 neuropathy.
   345. The method of any one of embodiments 338-344, wherein the subject or subjects have refused treatment with a platinum-based chemotherapeutic agent.
   346. The method of any one of embodiments 338 or 342-345, wherein the platinum-based chemotherapeutic agent is cisplatin.
   347. The method of any one of embodiments 338-346, wherein the MIBC is surgically operable.
   348. The method of embodiment 347, wherein the method further comprises a surgery.
   349. The method of embodiment 348, wherein at least one dosing cycle is initiated prior to the surgery.
   350. The method of embodiment 348 or 349, wherein at least 1, 2, or 3 dosing cycles are completed prior to the surgery.
   351. The method of any one of embodiments 348-350, wherein at least one dosing cycle is initiated between 4-6 weeks after the surgery.
   352. The method of embodiment 351, wherein 1-17 dosing cycles are completed after the surgery.
   353. The method of any one of embodiments 348-352, wherein the surgery is a cystectomy and/or lymph node dissection.
   354. The method of any one of embodiments 338-353, wherein the treating results in a pathological complete response (pCR).
   355. The method of any one of embodiments 338-354, wherein the treating results in an increase in the recurrence-free survival (RFS) of the subject or subjects as compared to a reference RFS time, an increase in the event-free survival (EFS) of the subject or subjects as compared to a reference EFS time, or an increase in the OS of the subject or subjects as compared to a reference OS time.
   356. The method of embodiment 355, wherein:
   (a) the reference RFS time is the median RFS time of a population of subjects who have received a treatment comprising a PD-1 axis binding antagonist without an anti-TIGIT antagonist antibody; (b) the reference EFS time is the median EFS time of a population of subjects who have received a treatment comprising a PD-1 axis binding antagonist without an anti-TIGIT antagonist antibody; or (c) the reference OS time is the median OS time of a population of subjects who have received a treatment comprising a PD-1 axis binding antagonist without an anti-TIGIT antagonist antibody.
   357. The method of any one of embodiments 338-356, wherein the treating results in a pathological downstaging.
   358. A method for treating a subject or population of subjects having an MIBC, the method comprising administering to the subject or population of subjects one or more dosing cycles of an anti-TIGIT antagonist antibody at a dose of between about 30 mg to about 1200 mg every three weeks and a PD-1 axis binding antagonist at a dose of between about 80 mg to about 1600 mg every three weeks, wherein the subject is cisplatin ineligible.
   359. A method for treating a subject or population of subjects having an MIBC, the method comprising administering to the subject or population of subjects one or more dosing cycles of an anti-TIGIT antagonist antibody at a dose of between about 30 mg to about 1200 mg every three weeks and a PD-1 axis binding antagonist at a dose of between about 80 mg to about 1600 mg every three weeks, wherein the treatment is a perioperative treatment.
   360. A method for treating a subject or population of subjects having an MIBC, the method comprising administering to the subject or population of subjects one or more dosing cycles of an anti-TIGIT antagonist antibody at a dose of between about 30 mg to about 1200 mg every three weeks and a PD-L1 binding antagonist at a dose of between about 80 mg to about 1600 mg every three weeks, wherein the subject is cisplatin ineligible.
   361. A method for treating a subject or population of subjects having an MIBC, the method comprising administering to the subject or population of subjects one or more dosing cycles of an anti-TIGIT antagonist antibody at a dose of between about 30 mg to about 1200 mg every three weeks and a PD-L1 binding antagonist at a dose of between about 80 mg to about 1600 mg every three weeks, wherein the treatment is a perioperative treatment.
   362. A method for treating a subject or population of subjects having an MIBC, the method comprising administering to the subject or population of subjects one or more dosing cycles of tiragolumab at a dose of between about 30 mg to about 1200 mg every three weeks and atezolizumab at a dose of between about 80 mg to about 1600 mg every three weeks, wherein the subject is cisplatin ineligible.
   363. A method for treating a subject or population of subjects having an MIBC, the method comprising administering to the subject or population of subjects one or more dosing cycles of tiragolumab at a dose of between about 30 mg to about 1200 mg every three weeks and atezolizumab at a dose of between about 80 mg to about 1600 mg every three weeks, wherein the treatment is a perioperative treatment.
   364. A method for treating a subject or population of subjects having an MIBC, the method comprising administering to the subject or population of subjects one or more dosing cycles of tiragolumab at a dose of about 600 mg every three weeks and atezolizumab at a dose of about 1200 mg every three weeks, wherein the subject or subjects are cisplatin ineligible.
   365. A method for treating a subject or population of subjects having an MIBC, the method comprising administering to the subject or population of subjects one or more dosing cycles of tiragolumab at a dose of about 600 mg every three weeks and atezolizumab at a dose of about 1200 mg every three weeks, wherein the treatment is a perioperative treatment.
   366. The method of any one of embodiments 338-365, wherein the treatment results in an ORR of the population of subjects of at least about 13.4% to about 15%.
   367. The method of any one of embodiments 338-366, wherein the treatment results in a median OS of the population of subjects of at least about 7.9 months to about 8.6 months.
   368. The method of any one of embodiments 338-367, wherein a detectable protein expression level of PD-L1 determined by an IHC assay comprising staining with anti-PD-L1 antibody SP142 has been determined.
   369. The method of embodiment 368, wherein the detectable protein expression level of PD-L1 is a PD-L1-positive tumor cell fraction with a proportion of tumor area occupied by PD-L1 expressing tumor-infiltrating immune cells (ICs) greater than or equal to 5%.
   370. The method of embodiment 336, wherein the bladder cancer is a urothelial carcinoma (UC).
   371. The method of embodiment 370, wherein the UC is a metastatic urothelial carcinoma (mUC).
   372. A method for treating a subject or population of subjects having an mUC, the method comprising administering to the subject or population of subjects a dosing regimen comprising one or more dosing cycles of an anti-TIGIT antagonist antibody at a dose of between about 30 mg to about 1200 mg every three weeks and a PD-1 axis binding antagonist at a dose of between about 80 mg to about 1600 mg every three weeks.
   373. A method for treating a subject or population of subjects having an mUC, the method comprising administering to the subject or population of subjects a dosing regimen comprising one or more dosing cycles of an anti-TIGIT antagonist antibody at a dose of between about 300 mg to about 800 mg every three weeks and a PD-1 axis binding antagonist at a dose of between about 900 mg to about 1500 mg every three weeks.
   374. A method for treating a subject or population of subjects having an mUC, the method comprising administering to the subject or population of subjects a dosing regimen comprising one or more dosing cycles of an anti-TIGIT antagonist antibody at a dose of between about 30 mg to about 1200 mg every three weeks and a PD-1 axis binding antagonist at a dose of between about 80 mg to about 1600 mg every three weeks, wherein the subject has not received a prior cancer immunotherapy.
   375. A method for treating a subject or population of subjects having an mUC, the method comprising administering to the subject or population of subjects a dosing regimen comprising one or more dosing cycles of an anti-TIGIT antagonist antibody at a dose of between about 30 mg to about 1200 mg every three weeks and a PD-1 axis binding antagonist at a dose of between about 80 mg to about 1600 mg every three weeks, wherein the treatment is a second-line treatment.
   376. The method any one of embodiments 372, 372, and 375, wherein the subject or subjects have not received a prior cancer immunotherapy.
   377. The method of any one of embodiments 372-374, wherein the treatment is a second-line treatment.
   378. The method of any one of embodiments 372-377, wherein the mUC has progressed during or following a platinum-containing therapy.
   379. The method of any one of embodiments 372-378, wherein the method further comprises administering to the subject or population of subjects a second dosing regimen after the subject or subjects have experienced disease progression or unacceptable toxicity.
   380. The method of embodiment 379, wherein the second dosing regimen comprises one or more dosing cycles of a PD-1 axis binding antagonist and an antibody-drug conjugate (ADC).
   381. The method of embodiment 380, wherein the ADC is (a) enfortumab vedotin or (b) sacituzumab govitecan.
   382. The method of embodiment 381, wherein (a) enfortumab vedotin is administered at a dose of 1.25 mg/kg every week for 2-weeks on/1 week off or (b) sacituzumab govitecan is administered at a dose of 10 mg/kg every week for 2-weeks on/1 week off.
   383. The method of embodiment 381 or 382, wherein (a) enfortumab vedotin is administered at a dose of 1.25 mg/kg on Days 1 and 8 of each 21-day cycle or (b) sacituzumab govitecan is administered at a dose of 10 mg/kg on Days 1 and 8 of each 21-day cycle.
   384. A method for treating a subject or population of subjects having an mUC, the method comprising administering to the subject or population of subjects a dosing regimen comprising one or more dosing cycles of an anti-TIGIT antagonist antibody at a dose of between about 30 mg to about 1200 mg every three weeks and a PD-L1 binding antagonist at a dose of between about 80 mg to about 1600 mg every three weeks.
   385. A method for treating a subject or population of subjects having an mUC, the method comprising administering to the subject or population of subjects a dosing regimen comprising one or more dosing cycles of tiragolumab at a dose of between about 30 mg to about 1200 mg every three weeks and atezolizumab at a dose of between about 80 mg to about 1600 mg every three weeks.
   386. A method for treating a subject or population of subjects having an mUC, the method comprising administering to the subject or population of subjects one or more dosing cycles of tiragolumab at a dose of about 600 mg every three weeks and atezolizumab at a dose of about 1200 mg every three weeks.
   387. A method for treating a subject or population of subjects having an mUC, the method comprising administering to the subject or population of subjects a first dosing regimen followed by a second dosing regimen, wherein:
   (a) the first dosing regimen comprises one or more dosing cycles of an anti-TIGIT antagonist antibody at a dose of between about 30 mg to about 1200 mg every three weeks and a PD-1 axis binding antagonist at a dose of between about 80 mg to about 1600 mg every three weeks; and
   (b) the second dosing regimen comprises one or more dosing cycles of the PD-1 axis binding antagonist at a dose of between about 80 mg to about 1600 mg every three weeks and (i) enfortumab vedotin is administered at a dose of 1.25 mg/kg every week for 2-weeks on/1 week off or (ii) sacituzumab govitecan is administered at a dose of 10 mg/kg every week for 2-weeks on/1 week off, wherein the second dosing regimen is administered to the subject or population of subjects after the subject or population of subjects have experienced disease progression or unacceptable toxicity during the first dosing regimen.
   388. A method for treating a subject or population of subjects having an mUC, the method comprising administering to the subject or population of subjects a first dosing regimen followed by a second dosing regimen, wherein:
   (a) the first dosing regimen comprises one or more dosing cycles of an anti-TIGIT antagonist antibody at a dose of between about 30 mg to about 1200 mg every three weeks and a PD-L1 binding antagonist at a dose of between about 80 mg to about 1600 mg every three weeks; and
   (b) the second dosing regimen comprises one or more dosing cycles of the PD-L1 binding antagonist at a dose of between about 80 mg to about 1600 mg every three weeks and (i) enfortumab vedotin is administered at a dose of 1.25 mg/kg every week for 2-weeks on/1 week off or (ii) sacituzumab govitecan is administered at a dose of 10 mg/kg every week for 2-weeks on/1 week off, wherein the second dosing regimen is administered to the subject after the subject has experienced disease progression or unacceptable toxicity during the first dosing regimen.
   389. A method for treating a subject or population of subjects having an mUC, the method comprising administering to the subject or population of subjects a first dosing regimen followed by a second dosing regimen, wherein:
   (a) the first dosing regimen comprises one or more dosing cycles of tiragolumab at a dose of about 600 mg every three weeks and atezolizumab at a dose of about 1200 mg every three weeks; and (b) the second dosing regimen comprises one or more dosing cycles of atezolizumab at a dose of about 1200 mg every three weeks and (i) enfortumab vedotin is administered at a dose of 1.25 mg/kg every week for 2-weeks on/1 week off or (ii) sacituzumab govitecan is administered at a dose of 10 mg/kg every week for 2-weeks on/1 week off, wherein the second dosing regimen is administered to the subject after the subject has experienced disease progression or unacceptable toxicity during the first dosing regimen.
   390. The method of any one of embodiments 372-389, wherein the treatment results in an ORR of the population of subjects of at least about 13.4% to at least about 31%.
   391. The method of any one of embodiments 372-390, wherein the treatment results in a median OS of the population of subjects of about 7.9 months to about 16.3 months.
   392. The method of embodiment 72, wherein the cancer is a pancreatic cancer.
   393. A method of treating a subject or a population of subjects having a pancreatic cancer, the method comprising administering to the subject or a population of subjects a dosing regimen comprising one or more 28-day dosing cycles of tiragolumab at a dose of about 420 mg on Days 1 and 15 of each 28-day dosing cycle, atezolizumab at a dose of about 840 mg on Days 1 and 15 of each 28-day dosing cycle, gemcitabine at a dose of about 1000 mg/m2 on Days 1, 8, and 15 of each 28-day dosing cycle, and nab-paclitaxel at a dose of about 125 mg/m2 on Days 1, 8, and 15 of each 28-day dosing cycle.
   394. The method of embodiment 392 or 393, wherein the pancreatic cancer is a pancreatic ductal adenocarcinoma (PDAC).
   395. The method of embodiment 394, wherein the PDAC is a metastatic PDAC.
   396. The method of any one of embodiments 392-395, wherein the subject or subjects have not received prior systemic therapy for metastatic PDAC.
   397. The method of any one of embodiments 392-396, wherein the treatment results in an ORR of the population of subjects of at least about 41.7% to about 46.7%.
   398. The method of any one of embodiments 392-397, wherein the treatment results in an increase in ORR of at least about 20% compared to a treatment comprising gemcitabine and nab-paclitaxel without an anti-TIGIT antagonist antibody and a PD-1 axis binding antagonist.
   399. The method of any one of embodiments 392-398, wherein the treatment results in a median PFS of the population of subjects of at least about 5.5 months to about 7 months.
   400. The method of any one of embodiments 392-399, wherein the treatment results in a median OS of the population of subjects of at least about 8.5 months to about 10.6 months.
   401. The method of embodiment 72, wherein the cancer is an esophageal cancer.
   402. A method for treating a subject or population of subjects having an advanced or metastatic esophageal cancer, the method comprising administering to the subject or population of subjects a dosing regimen comprising one or more 21-day dosing cycles of an anti-TIGIT antagonist antibody at a dose of between about 30 mg to about 1200 mg on Day 1 of each dosing cycle and a PD-1 axis binding antagonist at a dose of between about 80 mg to about 1600 mg on Day 1 of each dosing cycle.
   403. A method for treating a subject or population of subjects having an advanced or metastatic esophageal cancer, the method comprising administering to the subject or population of subjects a dosing regimen comprising one or more 21-day dosing cycles of an anti-TIGIT antagonist antibody at a dose of between about 30 mg to about 1200 mg on Day 1 of each dosing cycle and a PD-1 axis binding antagonist at a dose of between about 900 mg to about 1500 mg on Day 1 of each dosing cycle.
   404. A method for treating a subject or population of subjects having an esophageal cancer, the method comprising administering to the subject or population of subjects a dosing regimen comprising one or more 21-day dosing cycles of an anti-TIGIT antagonist antibody at a dose of between about 30 mg to about 1200 mg on Day 1 of each dosing cycle and a PD-1 axis binding antagonist at a dose of between about 80 mg to about 1600 mg on Day 1 of each dosing cycle, wherein the subject has been previously treated with a platinum-based chemotherapeutic agent and a non-platinum-based chemotherapeutic agent.
   405. A method for treating a subject or population of subjects having an esophageal cancer, the method comprising administering to the subject or population of subjects a dosing regimen comprising one or more 21-day dosing cycles of an anti-TIGIT antagonist antibody at a dose of between about 300 mg to about 800 mg on Day 1 of each dosing cycle and a PD-1 axis binding antagonist at a dose of between about 900 mg to about 1500 mg on Day 1 of each dosing cycle, wherein the subject has been previously treated with a platinum-based chemotherapeutic agent and a non-platinum-based chemotherapeutic agent.
   406. The method of embodiment 402 or 403, wherein the subject or subjects have been previously treated with a platinum-based chemotherapeutic agent and a non-platinum-based chemotherapeutic agent.
   407. The method of any one of embodiments 404-406, wherein the subject or subjects have experienced disease progression or unacceptable toxicity during the previous treatment.
   408. The method of embodiment 402 or 403, wherein the 21-day dosing cycles further comprise a platinum-based chemotherapeutic agent and a non-platinum-based chemotherapeutic agent.
   409. The method of embodiment 408, wherein the platinum-based chemotherapeutic agent is omitted from the dosing regimen after six doses.
   410. The method of any one of embodiments 404-409, wherein the platinum-based chemotherapeutic agent is cisplatin.
   411. The method of embodiment 410, wherein cisplatin is administered at a dose of about 80 mg/m2 on Day 1 of each dosing cycle.
   412. The method of any one of embodiments 404-411, wherein the non-platinum-based chemotherapeutic agent is an antimetabolite.
   413. The method of embodiment 412, wherein the antimetabolite is 5-fluorouracil.
   414. The method of embodiment 413, wherein 5-fluorouracil is administered at a dose of 800 mg/m2/24 hours on Days 1-5 of each 21-day cycle.
   415. The method of any one of embodiments 404-414, wherein the esophageal cancer is an advanced or metastatic esophageal cancer.
   416. The method of any one of embodiments 402, 403, 408, and 409, wherein the subject or subjects have had no prior treatment for metastatic esophageal cancer.
   417. A method for treating a subject or population of subjects having an advanced or metastatic esophageal cancer, the method comprising administering to the subject or population of subjects a dosing regimen comprising one or more 21-day dosing cycles of an anti-TIGIT antagonist antibody at a dose of between about 30 mg to about 1200 mg on Day 1 of each dosing cycle, a PD-1 axis binding antagonist at a dose of between about 80 mg to about 1600 mg on Day 1 of each dosing cycle, a platinum-based chemotherapeutic agent at a dose of about 80 mg/m2 on Day 1 of each dosing cycle, and a non-platinum-based chemotherapeutic agent at a dose of 800 mg/m2/24 hours on Days 1-5 of each 21-day cycle, wherein the platinum-based chemotherapeutic agent is omitted from the dosing regimen after six doses.
   418. A method for treating a subject or population of subjects having an advanced or metastatic esophageal cancer, the method comprising administering to the subject or population of subjects a dosing regimen comprising one or more 21-day dosing cycles of an anti-TIGIT antagonist antibody at a dose of about 600 mg on Day 1 of each dosing cycle, a PD-1 axis binding antagonist at a dose of about 1200 mg on Day 1 of each dosing cycle, cisplatin at a dose of about 80 mg/m2 on Day 1 of each dosing cycle, and 5-fluorouracil at a dose of 800 mg/m2/24 hours on Days 1-5 of each 21-day cycle, wherein cisplatin is omitted from the dosing regimen after six doses.
   419. A method for treating a subject or population of subjects having an advanced or metastatic esophageal cancer, the method comprising administering to the subject or population of subjects a dosing regimen comprising one or more 21-day dosing cycles of tiragolumab at a dose of about 600 mg on Day 1 of each dosing cycle, atezolizumab at a dose of about 1200 mg on Day 1 of each dosing cycle, cisplatin at a dose of about 80 mg/m2 on Day 1 of each dosing cycle, and 5-fluorouracil at a dose of 800 mg/m2/24 hours on Days 1-5 of each 21-day cycle, wherein cisplatin is omitted from the dosing regimen after six doses.
   420. A method for treating a subject or population of subjects having an advanced or metastatic esophageal cancer, the method comprising administering to the subject or population of subjects a first dosing regimen and a second dosing regimen, wherein:
   (a) the first dosing regimen comprises one or more 21-day dosing cycles of a platinum-based chemotherapeutic agent and a non-platinum-based chemotherapeutic agent, wherein the platinum-based chemotherapeutic agent is omitted from the dosing regimen after six doses; and
   (b) the second dosing regimen comprises one or more 21-day dosing cycles of an anti-TIGIT antagonist antibody at a dose of between about 30 mg to about 1200 mg on Day 1 of each dosing cycle and a PD-1 axis binding antagonist at a dose of between about 80 mg to about 1600 mg on Day 1 of each dosing cycle.
   421. A method for treating a subject or population of subjects having an advanced or metastatic esophageal cancer, the method comprising administering to the subject or population of subjects a first dosing regimen and a second dosing regimen, wherein:
   (a) the first dosing regimen comprises one or more 21-day dosing cycles of a platinum-based chemotherapeutic agent at a dose of about 80 mg/m2 on Day 1 of each dosing cycle and a non-platinum-based chemotherapeutic agent at a dose of 800 mg/m2/24 hours on Days 1-5 of each 21-day cycle, wherein the platinum-based chemotherapeutic agent is omitted from the dosing regimen after six doses; and
   (b) the second dosing regimen comprises one or more 21-day dosing cycles of an anti-TIGIT antagonist antibody at a dose of between about 30 mg to about 1200 mg on Day 1 of each dosing cycle and a PD-1 axis binding antagonist at a dose of between about 80 mg to about 1600 mg on Day 1 of each dosing cycle.
   422. A method for treating a subject or population of subjects having an advanced or metastatic esophageal cancer, the method comprising administering to the subject or population of subjects a first dosing regimen and a second dosing regimen, wherein:
   (a) the first dosing regimen comprises one or more 21-day dosing cycles of cisplatin at a dose of about 80 mg/m2 on Day 1 of each dosing cycle and 5-fluorouracil at a dose of 800 mg/m2/24 hours on Days 1-5 of each 21-day cycle, wherein cisplatin is omitted from the dosing regimen after six doses; and (b) the second dosing regimen comprises one or more 21-day dosing cycles of tiragolumab at a dose of about 600 mg on Day 1 of each dosing cycle and atezolizumab at a dose of about 1200 mg on Day 1 of each dosing cycle.
   423. The method of any one of embodiments 402-422, wherein the treatment results in an ORR of the population of subjects of at least about 14%.
   424. The method of any one of embodiments 47-423, wherein the anti-TIGIT antagonist antibody comprises the following hypervariable regions (HVRs):
   an HVR-H1 sequence comprising the amino acid sequence of SNSAAWN (SEQ ID NO: 1);
   an HVR-H2 sequence comprising the amino acid sequence of KTYYRFKWYSDYAVSVKG (SEQ ID NO: 2);
   an HVR-H3 sequence comprising the amino acid sequence of ESTTYDLLAGPFDY (SEQ ID NO: 3); an HVR-L1 sequence comprising the amino acid sequence of KSSQTVLYSSNNKKYLA (SEQ ID NO: 4); an HVR-L2 sequence comprising the amino acid sequence of WASTRES (SEQ ID NO: 5); and an HVR-L3 sequence comprising the amino acid sequence of QQYYSTPFT (SEQ ID NO: 6).
   425. The method of embodiment 424, wherein the anti-TIGIT antagonist antibody further comprises the following light chain variable region framework regions (FRs):
   an FR-L1 comprising the amino acid sequence of DIVMTQSPDSLAVSLGERATINC (SEQ ID NO: 7); an FR-L2 comprising the amino acid sequence of WYQQKPGQPPNLLIY (SEQ ID NO: 8);
   an FR-L3 comprising the amino acid sequence of GVPDRFSGSGSGTDFTLTISSLQAEDVAVYYC (SEQ ID NO: 9); and an FR-L4 comprising the amino acid sequence of FGPGTKVEIK (SEQ ID NO: 10).
   426. The method of embodiment 424, wherein the anti-TIGIT antagonist antibody further comprises the following heavy chain variable region FRs:
   an FR-H1 comprising the amino acid sequence of X1VQLQQSGPGLVKPSQTLSLTCAISGDSVS (SEQ ID NO: 11), wherein X1 is E or Q;
   an FR-H2 comprising the amino acid sequence of WIRQSPSRGLEWLG (SEQ ID NO: 12);
   an FR-H3 comprising the amino acid sequence of RITINPDTSKNQFSLQLNSVTPEDTAVFYCTR (SEQ ID NO: 13); and an FR-H4 comprising the amino acid sequence of WGQGTLVTVSS (SEQ ID NO: 14).
   427. The method of embodiment 426, wherein X1 is E.
   428. The method of embodiment 426, wherein X1 is Q.
   429. The method of any one of embodiments 424-428, wherein the anti-TIGIT antagonist antibody comprises:
   (a) a heavy chain variable (VH) domain comprising an amino acid sequence having at least 95% sequence identity to the amino acid sequence of SEQ ID NO: 17 or 18;
   (b) a light chain variable (VL) domain comprising an amino acid sequence having at least 95% sequence identity to the amino acid sequence of SEQ ID NO: 19; or
   (c) a VH domain as in (a) and a VL domain as in (b).
   430. The method of any one of embodiments 424-429, wherein the anti-TIGIT antagonist antibody comprises:
   (a) a VH domain comprising the amino acid sequence of SEQ ID NO: 17 or 18; and
   (b) a VL domain comprising the amino acid sequence of SEQ ID NO: 19.
   431. The method of any one of embodiments 424-427, 429, and 430, wherein the anti-TIGIT antagonist antibody comprises:
   (a) a VH domain comprising the amino acid sequence of SEQ ID NO: 17; and
   (b) a VL domain comprising the amino acid sequence of SEQ ID NO: 19.
   432. The method of any one of embodiments 424-427 and 429-431, wherein the anti-TIGIT antagonist antibody comprises:
   (a) a heavy chain comprising the amino acid sequence of SEQ ID NO: 33; and
   (b) a light chain comprising the amino acid sequence of SEQ ID NO: 34.
   433. The method of any one of embodiments 424-432, wherein the anti-TIGIT antagonist antibody is a monoclonal antibody.
   434. The method of any one of embodiments 424-433, wherein the anti-TIGIT antagonist antibody is a human antibody.
   435. The method of any one of embodiments 424-434, wherein the anti-TIGIT antagonist antibody is a full-length antibody.
   436. The method of any one of embodiments 424-427 and 429-436, wherein the anti-TIGIT antagonist antibody is tiragolumab.
   437. The method of any one of embodiments 47-112, 114-127, 136-174, 188-209, 227-253, 255, 265-269, 286-288, 291, 292, 295, 296, 301-332, 336-361, 370-384, 387, 388, 401-418, 420, 421, and 424-436, wherein the anti-TIGIT antagonist antibody is tiragolumab, vibostolimab, etigilimab, EOS084448,

### SGN-TGT, or TJ-T6.

438. The method of embodiment 437, wherein the anti-TIGIT antagonist antibody has intact Fc-mediated effector function.
439. The method of embodiment 437 or 438, wherein the anti-TIGIT antagonist antibody has enhanced Fc-mediated effector function.
440. The method of any one of embodiments 437-439, wherein the anti-TIGIT antagonist antibody is SGN-TGT.
441. The method of any one of embodiments 47-112, 114-127, 136-174, 188-209, 227-253, 255, 265-269, 286-288, 291, 292, 295, 296, 301-332, 336-361, 370-384, 387, 388, 401-418, 420, and 421, wherein the anti-TIGIT antagonist antibody is domvanalimab, BMS-986207, ASP8374, or COM902.
442. The method of any one of embodiments 424-431 and 441, wherein the anti-TIGIT antagonist antibody does not have Fc-mediated effector function.
443. The method of any one of embodiments 47-442, wherein the anti-TIGIT antagonist antibody is an IgG class antibody.
444. The method of embodiment 443, wherein the IgG class antibody is an IgG1 subclass antibody.
445. The method of embodiment 444, wherein the anti-TIGIT antagonist antibody is tiragolumab, vibostolimab, etigilimab, EOS084448, SGN-TGT, TJ-T6, BGB-A1217, AB308, domvanalimab, or BMS-986207.
446. The method of any one of embodiments 47-112, 114-127, 136-174, 188-209, 227-253, 255, 265-269, 286-288, 291, 292, 295, 296, 301-332, 336-361, 370-384, 387, 388, 401-418, 420, and 421, wherein the IgG class antibody is an IgG4 subclass antibody.
447. The method of embodiment 446, wherein the anti-TIGIT antagonist antibody is ASP8374 or COM902.
448. The method of any one of embodiments 424-434, wherein the anti-TIGIT antagonist antibody is an antibody fragment that binds TIGIT selected from the group consisting of Fab, bis-Fab, Fab′, Fab′-SH, Fv, single chain variable fragment (scFv), and (Fab′)2 fragments.
449. The method of any one of embodiments 424-448, wherein the anti-TIGIT antagonist antibody is a monospecific antibody.
450. The method of any one of embodiments 424-448, wherein the anti-TIGIT antagonist antibody is a multispecific antibody.
451. The method of embodiment 450, wherein the multispecific antibody is a bispecific antibody.
452. The method of any one of embodiments 424-451, wherein the PD-1 axis binding antagonist is selected from the group consisting of a PD-L1 binding antagonist, a PD-1 binding antagonist, and a PD-L2 binding antagonist.
453. The method of embodiment 452, wherein the PD-1 axis binding antagonist is a PD-L1 binding antagonist.
454. The method of embodiment 453, wherein the PD-L1 binding antagonist inhibits the binding of PD-L1 to one or more of its ligand binding partners.
455. The method of embodiment 454, wherein the PD-L1 binding antagonist inhibits the binding of PD-L1 to PD-1, B7-1, or both PD-1 and B7-1.
456. The method of any one of embodiments 453-455, wherein the PD-L1 binding antagonist is an anti-PD-L1 antagonist antibody.
457. The method of embodiment 456, wherein the anti-PD-L1 antagonist antibody is atezolizumab, MDX-1105, durvalumab, avelumab, SHR-1316, CS1001, envafolimab, TQB2450, ZKAB001, LP-002, CX-072,
IMC-001, KL-A167, APL-502, cosibelimab, lodapolimab, FAZ053, TG-1501, BGB-A333, BCD-135, AK-106, LDP, GR1405, HLX20, MSB2311, RC98, PDL-GEX, KD036, KY1003, YBL-007, or HS-636. 458. The method of embodiment 457, wherein the anti-PD-L1 antagonist antibody is atezolizumab.
459. The method of embodiment 456, wherein the anti-PD-L1 antagonist antibody comprises the following HVRs:
an HVR-H1 sequence comprising the amino acid sequence of GFTFSDSWIH (SEQ ID NO: 20);
an HVR-H2 sequence comprising the amino acid sequence of AWISPYGGSTYYADSVKG (SEQ ID NO: 21);
an HVR-H3 sequence comprising the amino acid sequence of RHWPGGFDY (SEQ ID NO: 22);
an HVR-L1 sequence comprising the amino acid sequence of RASQDVSTAVA (SEQ ID NO: 23);
an HVR-L2 sequence comprising the amino acid sequence of SASFLYS (SEQ ID NO: 24); and an HVR-L3 sequence comprising the amino acid sequence of QQYLYHPAT (SEQ ID NO: 25).
460. The method of embodiment 459, wherein the anti-PD-L1 antagonist antibody comprises: (a) a heavy chain variable (VH) domain comprising an amino acid sequence having at least 95% sequence identity to the amino acid sequence of SEQ ID NO: 26;
(b) a light chain variable (VL) domain comprising an amino acid sequence having at least 95% sequence identity to the amino acid sequence of SEQ ID NO: 27; or
(c) a VH domain as in (a) and a VL domain as in (b).
461. The method of embodiment 460, wherein the anti-PD-L1 antagonist antibody comprises: (a) a VH domain comprising the amino acid sequence of SEQ ID NO: 26; and
(b) a VL domain comprising the amino acid sequence of SEQ ID NO: 27.
462. The method of embodiment 461, wherein the anti-PD-L1 antagonist antibody comprises: (a) a heavy chain comprising the amino acid sequence of SEQ ID NO: 28; and
(b) a light chain comprising the amino acid sequence of SEQ ID NO: 29.
463. The method of any one of embodiments 459-462, wherein the anti-PD-L1 antagonist antibody is a monoclonal antibody.
464. The method of any one of embodiments 459-463, wherein the anti-PD-L1 antagonist antibody is a humanized antibody.
465. The method of embodiment 463 or 464, wherein the anti-PD-L1 antagonist antibody is a full-length antibody.
466. The method of any one of embodiments 459-464, wherein the anti-PD-L1 antagonist antibody is an antibody fragment that binds PD-L1 selected from the group consisting of Fab, Fab′, Fab′-SH, Fv, scFv, and (Fab′)2 fragments.
467. The method of any one of embodiments 459-465, wherein the anti-PD-L1 antagonist antibody is an IgG class antibody.
468. The method of embodiment 467, wherein the IgG class antibody is an IgG1 subclass antibody.
469. The method of embodiment 452, wherein the PD-1 axis binding antagonist is a PD-1 binding antagonist.
470. The method of embodiment 469, wherein the PD-1 binding antagonist inhibits the binding of PD-1 to one or more of its ligand binding partners.
471. The method of embodiment 470, wherein the PD-1 binding antagonist inhibits the binding of PD-1 to PD-L1, PD-L2, or both PD-L1 and PD-L2.
472. The method of any one of embodiments 469-471, wherein the PD-1 binding antagonist is an anti-PD-1 antagonist antibody.
473. The method of embodiment 472, wherein the anti-PD-1 antagonist antibody is nivolumab, pembrolizumab, MEDI-0680, spartalizumab, cemiplimab, BGB-108, prolgolimab, camrelizumab, sintilimab, tislelizumab, toripalimab, dostarlimab, retifanlimab, sasanlimab, penpulimab, CS1003, HLX10,
SCT-110A, zimberelimab, balstilimab, genolimzumab, BI 754091, cetrelimab, YBL-006, BAT1306, HX008, budigalimab, AMG 404, CX-188, JTX-4014, 609A, Sym021, LZM009, F520, SG001, AM0001, ENUM 244C8, ENUM 388D4, STI-1110, AK-103, or hAb21.
474. The method of any one of embodiments 469-471, wherein the PD-1 binding antagonist is an Fc fusion protein.
475. The method of embodiment 474, wherein the Fc fusion protein is AMP-224.
476. The method of any one of embodiments 80-112, 114-127 and 309-312, wherein the method comprises administering to the subject or population of subjects the anti-TIGIT antagonist antibody at a dose of between about 30 mg to about 1200 mg every three weeks.
477. The method of any one of embodiments 51, 54, 60, 62, 63, 65-67, 80-112, 114-127, 136-140, 169-174, 189, 191, 267, 269, 286, 288, 291, 295, 301, 302, 303, 313, 315, 338, 340, 358-361, 372, 384, 387, 388, 402-404, 417, 420, 421, and 476, wherein the method comprises administering to the subject the anti-TIGIT antagonist antibody at a dose of about 600 mg every three weeks.
478. The method of any one of embodiments 51, 54, 60, 62, 63, 65-67, 80-112, 114-127, 136-140, 169-174, 189, 191, 267, 269, 286, 288, 291, 295, 301, 302, 303, 309, 313, 315, 338, 340, 358-361, 372, 384, 387, 388, 402-404, 417, 420, 421, and 476, wherein the method comprises administering to the subject or population of subjects the anti-TIGIT antagonist antibody at a tiered dose based on a subject's body weight, wherein the subject has a body weight of:
(a) less than or equal to 15 kg, and the anti-TIGIT antagonist antibody is administered at a dose of about 300 mg every three weeks;
(b) greater than 15 kg and less than or equal to 40 kg, and the anti-TIGIT antagonist antibody is administered at a dose of about 400 mg every three weeks; or
(c) greater than 40 kg, and the anti-TIGIT antagonist antibody is administered at a dose of about 600 mg every three weeks.
479. The method of any one of embodiments 47, 48, 77, 78, 82, and 83, wherein the method comprises administering to the subject or population of subjects the anti-TIGIT antagonist antibody at a dose of about 700 mg to about 1000 mg every four weeks.
480. The method of any one of embodiments 47, 48, 59, 67, 323, and 479, wherein the method comprises administering to the subject the anti-TIGIT antagonist antibody at a dose of about 840 mg every four weeks.
481. The method of any one of embodiments 49, 50, and 104, wherein the method comprises administering to the subject or population of subjects the anti-TIGIT antagonist antibody at a dose of about 300 mg to about 600 mg every two weeks.
482. The method of any one of embodiments 49, 50, 227, 251-253, 255, and 481, wherein the method comprises administering to the subject the anti-TIGIT antagonist antibody at a dose of about 420 mg every two weeks.
483. The method of any one of embodiments 47-482, wherein the dose of the anti-TIGIT antagonist antibody is a fixed dose.
484. The method of any one of embodiments 54, 62, 63, 65-67, 80-109, and 27-2698, wherein the method comprises administering to the subject or population of subjects the PD-1 axis binding antagonist at a dose of between about 900 mg to about 1500 mg every three weeks.
485. The method of any one of embodiments 54, 60, 62, 63, 65-67, 80-112, 114-127, 136-140, 169-172, 189, 191, 267, 269, 286, 288, 291, 295, 301-303, 309, 311, 313, 315, 338, 340, 358-361, 372-375, 384, 387, 388, 402-405, 417, 420, 421, and 484, wherein the method comprises administering to the subject the PD-1 axis binding antagonist at a dose of about 1200 mg every three weeks.
486. The method of any one of embodiments 47, 50, 80-109, and 324, wherein the method comprises administering to the subject or population of subjects the PD-1 axis binding antagonist at a dose of about 1400 mg to 2000 mg every four weeks.
487. The method of any one of embodiments 47, 50, 59, 67, 324, and 479, wherein the method comprises administering to the subject the PD-1 axis binding antagonist at a dose of about 1680 mg every four weeks.
488. The method of embodiment 48, 49, 80-109, and 481, wherein the method comprises administering to the subject or population of subjects the PD-1 axis binding antagonist at a dose of between about 600 mg to about 1200 mg every two weeks.
489. The method of any one of embodiments 48, 49, 80-109, 227, 251-253, and 481, wherein the method comprises administering to the subject the PD-1 axis binding antagonist at a dose of about 840 mg every two weeks.
490. The method of any one of embodiments 47-489, wherein the dose of the PD-1 axis binding antagonist is a fixed dose.
491. The method of embodiment 60 or 61, wherein the method comprises administering to the subject pembrolizumab at a fixed dose of about 200 mg every three weeks or 400 mg every six weeks.
492. The method of any one of embodiments 54, 60, 62, 63, 65, 80-109, and 191, wherein the length of each of the one or more dosing cycles is 21 days.
493. The method of any one of embodiments 47, 59, 67, 80-109, and 309-311, wherein the length of each of the one or more dosing cycles is 28 days.
494. The method of any one of embodiments 49, 80-109, and 309-311 wherein the length of each of the one or more dosing cycles is 14 days.
495. The method of any one of embodiments 47-494, wherein the method comprises administering to the subject or population of subjects the anti-TIGIT antagonist antibody on about Day 1 of each of the one or more dosing cycles.
496. The method of any one of embodiments 47-495, wherein the method comprises administering to the subject or population of subjects the PD-1 axis binding antagonist on about Day 1 of each of the one or more dosing cycles.
497. The method of embodiment 321 or 493, wherein the method comprises administering to the subject the anti-TIGIT antagonist antibody on about Day 15 of each of the one or more dosing cycles.
498. The method of embodiment 322 or 493, wherein the method comprises administering to the subject the PD-1 axis binding antagonist on about Day 15 of each of the one or more dosing cycles.
499. The method of any one of embodiments 47-498, wherein the method comprises administering to the subject or population of subjects the PD-1 axis binding antagonist before the anti-TIGIT antagonist antibody or the anti-TIGIT antagonist antibody before the PD-1 axis binding antagonist.
500. The method of embodiment 499, wherein the method comprises a first observation period following administration of the PD-1 axis binding antagonist or the anti-TIGIT antagonist antibody.
501. The method of embodiment 500, wherein the first observation period is between about 30 minutes to about 60 minutes in length.
502. The method of embodiment 500 or 501, wherein the method comprises a second observation period following administration of the anti-TIGIT antagonist antibody or the PD-1 axis binding antagonist.
503. The method of embodiment 502, wherein the second observation period is between about 30 minutes to about 60 minutes in length.
504. The method of any one of embodiments 47-503, wherein the method comprises administering to the subject or population of subjects the PD-1 axis binding antagonist simultaneously with the anti-TIGIT antagonist antibody.
505. The method of any one of embodiments 47-504, wherein the method comprises administering to the subject or population of subjects the anti-TIGIT antagonist antibody and PD-1 axis binding antagonist intravenously.
506. The method of embodiment 505, wherein the method comprises administering to the subject or population of subjects the PD-1 axis binding antagonist by intravenous infusion over 30±10 minutes and/or over 60±10 minutes.
507. The method of embodiment 505 or 506, wherein the method comprises administering to the subject or population of subjects the anti-TIGIT antagonist antibody by intravenous infusion over 30±10 minutes and/or over 60±10 minutes.
508. The method of any one of embodiments 47-114 and 116-507, wherein a PD-L1 expression level of a tumor sample obtained from the subject or subjects have been determined.
509. The method of embodiment 508, wherein the tumor sample obtained from the subject or subjects have been determined to have a detectable expression level of PD-L1.
510. The method of embodiment 509, wherein the detectable expression level of PD-L1 is a detectable protein expression level of PD-L1.
511. The method of embodiment 510, wherein the detectable protein expression level of PD-L1 has been determined by an immunohistochemical (IHC) assay comprising staining with an anti-PD-L1 antibody suitable for staining.
512. The method of embodiment 511, wherein the anti-PD-L1 antibody suitable for staining is the anti-PD-L1 antibody SP263, SP142, 22C3, or 28-8.
513. The method of embodiment 511 or 512, wherein the detectable protein expression level of PD-L1 is determined using a Ventana SP263 IHC assay, a pharmDx 22C3 IHC assay, a Ventana SP142 IHC assay, or a pharmDx 28-8 IHC assay.
514. The method of any one any one of embodiments 47-109, 188, 220-265, and 307-423, wherein a detectable protein expression level of PD-L1 determined by an IHC assay comprising staining with anti-PD-L1 antibody SP263 has been determined.
515. The method of any one any one of embodiments 47-220, 265-338, and 370-423, wherein a detectable protein expression level of PD-L1 determined by an IHC assay comprising staining with anti-PD-L1 antibody SP142 has been determined.
516. The method of any one any one of embodiments 47-113, 136-187 and 220-423, wherein a detectable protein expression level of PD-L1 determined by an IHC assay comprising staining with anti-PD-L1 antibody 22C3 has been determined.
517. The method of any one any one of embodiments 47-114 and 116-423, wherein a detectable protein expression level of PD-L1 determined by an IHC assay comprising staining with anti-PD-L1 antibody 28-8 has been determined.
518. The method of embodiment 514, wherein the detectable protein expression level of PD-L1 is a tumor-associated immune-cell (TIC) of greater than or equal to 5% in the tumor sample.
519. The method of embodiment 514, wherein the detectable protein expression level of PD-L1 is a TIC of greater than or equal to 5% and less than 20% in the tumor sample.
520. The method of embodiment 514, wherein the detectable protein expression level of PD-L1 is a TIC of greater than or equal to 10% in the tumor sample.
521. The method of embodiment 514, wherein the detectable protein expression level of PD-L1 is a TIC of greater than or equal to 20% in the tumor sample.
522. The method of embodiment 514, wherein the detectable protein expression level of PD-L1 is a TIC of greater than or equal to 10% and less than 50% in the tumor sample.
523. The method of embodiment 514, wherein the detectable protein expression level of PD-L1 is a TIC of greater than or equal to 50% in the tumor sample.
524. The method of any one of embodiments 514, 516, and 517, wherein the detectable protein expression level of PD-L1 is a PD-L1-positive tumor cell fraction of greater than or equal to 1%.
525. The method of any one of embodiments 514, 516, and 517, wherein the detectable protein expression level of PD-L1 is a PD-L1-positive tumor cell fraction of greater than or equal to 30%.
526. The method of any one of embodiments 514, 516, and 517, wherein the detectable protein expression level of PD-L1 is a PD-L1-positive tumor cell fraction of greater than or equal to 1% and less than 50% in the tumor sample.
527. The method of any one of embodiments 514, 516, and 517, wherein the detectable protein expression level of PD-L1 is a PD-L1-positive tumor cell fraction of greater than or equal to 50%.
528. The method of embodiment 515, wherein the PD-L1-positive tumor cell fraction is a proportion of tumor area occupied by PD-L1 expressing tumor-infiltrating immune cells (ICs).
529. The method of embodiment 515, wherein the proportion of tumor area occupied by PD-L1 expressing tumor-infiltrating ICs is greater than or equal to 1%.
530. The method of embodiment 515, wherein the proportion of tumor area occupied by PD-L1 expressing tumor-infiltrating ICs is greater than or equal to 5%.
531. The method of embodiment 516, wherein the detectable protein expression level of PD-L1 is a combined positive score (CPS) of greater than or equal to 1.
532. The method of embodiment 516, wherein the detectable protein expression level of PD-L1 is a CPS of greater than or equal to 10.
533. The method of embodiment 516, wherein the detectable protein expression level of PD-L1 is a CPS of greater than or equal to 20.
534. The method of any one of embodiments 528-530, wherein the IHC assay is a Ventana SP142 IHC assay.
535. The method of embodiment 509, wherein the detectable expression level of PD-L1 is a detectable nucleic acid expression level of PD-L1.
536. The method of embodiment 535, wherein the detectable nucleic acid expression level of PD-L1 has been determined by RNA-seq, RT-qPCR, qPCR, multiplex qPCR or RT-qPCR, microarray analysis,
SAGE, MassARRAY technique, ISH, or a combination thereof.
537. The method of any one of embodiments 47-423, wherein the treating results in an increase in overall survival (OS) of the subject as compared to a reference OS time and/or progression-free survival (PFS) of the subject as compared to a reference PFS time.
538. The method of embodiment 537, wherein:
(a) the reference OS time is the median OS time of a population of subjects who have received a treatment comprising a PD-1 axis binding antagonist without an anti-TIGIT antagonist antibody or a treatment comprising an anti-TIGIT antagonist antibody without a PD-1 axis binding antagonist; and/or
(b) the reference PFS time is the median PFS time of a population of subjects who have received a treatment comprising a PD-1 axis binding antagonist without an anti-TIGIT antagonist antibody or a treatment comprising an anti-TIGIT antagonist antibody without a PD-1 axis binding antagonist.
539. The method of any one of embodiments 52, 158, and 228, wherein the one or more chemotherapeutic agents are one or more platinum-based chemotherapeutic agents and/or one or more non-platinum-based chemotherapeutic agents.
540. The method of embodiment 539, wherein the platinum-based chemotherapeutic agents is carboplatin or cisplatin.
541. The method of embodiment 69, 92, 127, 131, 249, and 540, wherein the carboplatin is administered at a dose sufficient to achieve an AUC=5 mg/ml/min or an AUC=6 mg/ml/min.
542. The method of embodiment 69, 127, 162, 164, 166, 168, and 540, wherein the cisplatin is administered at a dose of about 75 mg/m2 or 80 mg/m2.
543. The method of any one of embodiments 539-542, wherein the one or more non-platinum-based chemotherapeutic agents are an antimetabolite, a taxane, or a topoisomerase II inhibitor.
544. The method of any one of embodiments 62, 173, 174, and 543, wherein the antimetabolite is pemetrexed, gemcitabine, capecitabine, or 5-fluorouracil.
545. The method of any one of embodiments 69, 127, 162, and 544, wherein the pemetrexed is administered at a dose of about 500 mg/m2.
546. The method of embodiment any one of embodiments 63, 64, 162, 398, and 544, wherein the gemcitabine is administered at a dose of about 1000 mg/m2 or about 1250 mg/m2.
547. The method of embodiment 544, wherein the antimetabolite is capecitabine.
548. The method of embodiment 544 or 547, wherein the capecitabine is administered at a dose of about 1250 mg/m2.
549. The method of any one of embodiments 173, 174, 229, 230, 239, 240, and 543-548, wherein the taxane is paclitaxel or nab-paclitaxel.
550. The method of any one of embodiments 69, 162, and 549, wherein the paclitaxel is administered at a dose of about 175 mg/m2 or about 200 mg/m2.
551. The method of any one of embodiments 63, 64, 249, 398, and 549, wherein the nab-paclitaxel is administered at a dose of about 100 mg/m2.
552. The method of any one of embodiments 80-91, 229, 230, 239, 241, 251-255, and 543-551, wherein the topoisomerase II inhibitor is etoposide, teniposide, doxorubicin, daunorubicin, mitoxantrone, amsacrine, an ellipticine, aurintricarboxylic acid, or HU-331.
553. The method of any one of embodiments 69, 92, and 552, wherein the etoposide is administered at a dose of about 100 mg/m2.
554. The method of any one of embodiments 543-553, wherein the one or more chemotherapeutic agents are each administered once per week, once every two weeks, once every three weeks, twice every three weeks, once every four weeks, twice every four weeks, or three times every four weeks.
555. The method of any one of embodiments 543-554, wherein the one or more chemotherapeutic agents are administered on Day 1 of one or more dosing cycles.
556. The method of any one of embodiments 544-555, wherein the gemcitabine is administered three times every four weeks.
557. The method of any one of embodiments 544-556, wherein the gemcitabine is administered on Days 1, 8, and/or 15 of one or more dosing cycles.
558. The method of any one of embodiments 544-557, wherein the capecitabine is administered daily for two weeks.
559. The method of any one of embodiments 544-558, wherein the capecitabine is administered on Days 1-14 of one or more dosing cycles.
560. The method of any one of embodiments 549-559, wherein the nab-paclitaxel is administered three times every four weeks.
561. The method of any one of embodiments 549-560, wherein the nab-paclitaxel is administered on Days 1, 8, and 15 of one or more dosing cycles.
562. The method of any one of embodiments 552-561, wherein the etoposide is administered on Days 1-3 every three weeks.
563. The method of any one of embodiments 552-562, wherein the etoposide is administered on Days 1-3 of one or more dosing cycles.
564. The method of any one of embodiments 543-563, wherein the one or more chemotherapeutic agents are administered before the PD-1 axis binding antagonist and/or the anti-TIGIT antagonist antibody.
565. The method of any one of embodiments 543-564, wherein the one or more chemotherapeutic agents are administered after the PD-1 axis binding antagonist and/or the anti-TIGIT antagonist antibody.
566. The method of any one of embodiments 543-565, wherein the one or more chemotherapeutic agents are administered intravenously or orally.
567. The method of any one of embodiments 65, 310, and 311, wherein the VEGF antagonist is administered at a dose of about 5 mg/kg to about 25 mg/kg every three weeks.
568. The method of embodiment 312 or 567, wherein the VEGF antagonist is administered at a dose of about 10 mg/kg to about 20 mg/kg every three weeks.
569. The method of embodiment 318 or 568, wherein the VEGF antagonist is administered at a dose of about 15 mg/kg every three weeks.
570. The method of any one of embodiments 65, 310-312, 318-320, and 567-569, wherein the VEGF antagonist is an anti-VEGF antibody.
571. The method of embodiment 570, wherein the anti-VEGF antibody comprises the VH domain and the VL domain of bevacizumab.
572. The method of embodiment 570 or 571, wherein the anti-VEGF antibody is bevacizumab.
573. The method of any one of embodiments 65, 310-312, and 318-320, wherein the anti-TIGIT antagonist antibody is tiragolumab, the PD-1 axis binding antagonist is atezolizumab, and the VEGF antagonist is bevacizumab.
574. The method of any one of embodiments 65, 310-312, 318-320, and 567-573, wherein the method comprises administering to the subject the VEGF antagonist on Day 1 of one or more dosing cycles.
575. The method of any one of embodiments 318-320, wherein the method comprises administering to the subject the VEGF antagonist on Day 15 of one or more dosing cycles.
576. The method of any one of embodiments 65, 310-312, and 318-320, and 567-575, wherein the VEGF antagonist is administered intravenously.
577. The method of embodiment 576, wherein the VEGF antagonist is administered to the subject by intravenous infusion over 90±15 minutes.
578. The method of any one of embodiments 65, 310-312, 318-320, and 567-577, wherein the method comprises administering to the subject the PD-1 axis binding antagonist before the VEGF antagonist and the VEGF antagonist before the anti-TIGIT antagonist antibody.
579. The method of embodiment 578, wherein the method comprises a first observation period following administration of the PD-1 axis binding antagonist, a second observation period following administration of the VEGF antagonist, and a third observation period following administration of the anti-TIGIT antagonist antibody.
580. The method of embodiment 579, wherein the first observation period, the second observation period, and the third observation period are each between about 30 minutes to about 120 minutes in length.
581. The method of any one of embodiments 113, 128, 129-131, 175-182, 210-214, 221, 256, 257, 270, 289, 290, 293, 294, 297, 298, 333, 362-365, 385, 386, 389, 393, 419, and 422, wherein tiragolumab and atezolizumab are combined in an IV bag prior to administration.
582. The method of any one of embodiments 47-581, wherein the subject is a human.
583. A kit comprising a PD-1 axis binding antagonist and/or an anti-TIGIT antagonist antibody for treating a subject having a cancer according to the method of any one of embodiments 47-190, 192-208, 210-211, 213, 215-268, 270-294, and 299-582.
584. A kit comprising a PD-1 axis binding antagonist for use in combination with an anti-TIGIT antagonist antibody for treating a subject having a cancer according to the method of any one of embodiments 47-190, 192-208, 210-211, 213, 215-268, 270-294, and 299-582.
585. The kit of embodiment 584, wherein the kit further comprises an anti-TIGIT antagonist antibody.
586. The kit of any one of embodiments 583-585, wherein the anti-TIGIT antagonist antibody is tiragolumab.
587. The kit of any one of embodiments 583-586, wherein the PD-1 axis binding antagonist is atezolizumab.
588. A kit comprising an anti-TIGIT antagonist antibody for use in combination with a PD-1 axis binding antagonist for treating a subject having a cancer according to the method of any one of embodiments 47-190, 192-208, 210-211, 213, 215-268, 270-294, and 299-582.
589. The kit of embodiment 588, wherein the kit further comprises a PD-1 axis binding antagonist.
590. The kit of embodiment 588 or 589, wherein the PD-1 axis binding antagonist is atezolizumab.
591. The kit of any one of embodiments 588-590, wherein the anti-TIGIT antagonist antibody is tiragolumab.
592. The kit of any one of embodiments 583-591, wherein the kit further comprises one or more chemotherapeutic agents.
593. The kit of embodiment 592, wherein the one or more chemotherapeutic agents are one or more platinum-based chemotherapeutic agents and/or one or more non-platinum-based chemotherapeutic agents.
594. The kit of embodiment 593, wherein the one or more platinum-based chemotherapeutic agents are carboplatin or cisplatin.
595. The kit of embodiment 593 or 594, wherein the one or more non-platinum-based chemotherapeutic agent are an antimetabolite, a taxane, or a topoisomerase II inhibitor.
596. The kit of embodiment 595, wherein the antimetabolite is pemetrexed, gemcitabine, capecitabine, or 5-fluorouracil.
597. The kit of embodiment 595 or 596, wherein the taxane is paclitaxel or nab-paclitaxel.
598. The kit of any one of embodiments 595-597, wherein the topoisomerase II inhibitor is etoposide, teniposide, doxorubicin, daunorubicin, mitoxantrone, amsacrine, an ellipticine, aurintricarboxylic acid, or HU-331.
599. The kit of any one of embodiments 583-598, wherein the kit further comprises a VEGF antagonist.
600. The kit of embodiment 599, wherein the VEGF antagonist is an anti-VEGF antibody.
601. The kit of embodiment 600, wherein the anti-VEGF antibody is bevacizumab.
602. An anti-TIGIT antagonist antibody and a PD-1 axis binding antagonist for use in a method of treating a subject or population of subjects having a cancer, wherein the method is according to any one of embodiments 47-190, 192-208, 210-211, 213, 215-268, 270-294, and 299-582.
603. Use of an anti-TIGIT antagonist antibody in the manufacture of a medicament for treating a subject or population of subjects having a cancer in combination with a PD-1 axis binding antagonist, wherein the treatment is according to the method of any one of embodiments 47-190, 192-208, 210-211, 213, 215-268, 270-294, and 299-582.
604. The use of embodiment 603, wherein the anti-TIGIT antagonist antibody and the PD-1 axis binding antagonist are provided in separate formulations.
605. The use of embodiment 603, wherein the anti-TIGIT antagonist antibody and the PD-1 axis binding antagonist are provided in a single formulation.

Although the foregoing invention has been described in some detail by way of illustration and example for purposes of clarity of understanding, the descriptions and examples should not be construed as limiting the scope of the invention. The disclosures of all patent and scientific literature cited herein are expressly incorporated in their entirety by reference.

1-503. (canceled)

504. A method of treating a subject or population of subjects having a lung cancer, the method comprising administering to the subject or population of subjects a dosing regimen comprising one or more dosing cycles of an effective amount of an anti-TIGIT antagonist antibody, a PD-1 axis binding antagonist, a platinum-based chemotherapeutic agent, and a topoisomerase II inhibitor, wherein the treatment extends progression-free survival (PFS) or overall survival (OS) of the subject as compared to treatment with the PD-1 axis binding antagonist, the platinum-based chemotherapeutic agent, and the topoisomerase II inhibitor without the anti-TIGIT antagonist antibody.

505. The method of claim 504, wherein the anti-TIGIT antagonist antibody, PD-1 axis binding antagonist, platinum-based chemotherapeutic agent, and topoisomerase II inhibitor are administered in each of four initial dosing cycles.

506. The method of claim 504, wherein the anti-TIGIT antagonist antibody and the PD-1 axis binding antagonist are further administered in one or more additional cycles following the fourth initial dosing cycle.

507. The method of claim 504, wherein the platinum-based chemotherapeutic agent and the topoisomerase II inhibitor are omitted from each of the one or more additional dosing cycles.

508. The method of claim 504, wherein the platinum-based chemotherapeutic agent is carboplatin and the topoisomerase II inhibitor is etoposide.

509. The method of claim 504, wherein the lung cancer is extensive stage small cell lung cancer (ES-SCLC).

510. The method of claim 509, wherein the subject or subjects are treatment-naïve for ES-SCLC and/or wherein the subject or subjects do not have a presence or history of brain metastases.

511. The method of claim 504, wherein the anti-TIGIT antagonist antibody is a monoclonal antibody and/or a human antibody.

512. The method of claim 504, wherein (a) the anti-TIGIT antagonist antibody is a full-length antibody or (b) the anti-TIGIT antagonist antibody is an antibody fragment that binds TIGIT selected from the group consisting of Fab, Fab′, Fab′-SH, Fv, single chain variable fragment (scFv), and (Fab′)₂ fragments.

513. The method of claim 504, wherein the anti-TIGIT antagonist antibody has intact Fc-mediated effector function.

514. The method of claim 504, wherein:
(a) the anti-TIGIT antagonist antibody is tiragolumab, vibostolimab, etigilimab, or EOS084448;
(b) the anti-TIGIT antagonist antibody comprises the following hypervariable regions (HVRs):
an HVR-H1 sequence comprising the amino acid sequence of SNSAAWN (SEQ ID NO: 1);
an HVR-H2 sequence comprising the amino acid sequence of KTYYRFKWYSDYAVSVKG (SEQ ID NO: 2);
an HVR-H3 sequence comprising the amino acid sequence of ESTTYDLLAGPFDY (SEQ ID NO: 3);
an HVR-L1 sequence comprising the amino acid sequence of KSSQTVLYSSNNKKYLA (SEQ ID NO: 4);
an HVR-L2 sequence comprising the amino acid sequence of WASTRES (SEQ ID NO: 5); and
an HVR-L3 sequence comprising the amino acid sequence of QQYYSTPFT (SEQ ID NO: 6);
(c) the anti-TIGIT antagonist antibody comprises a VH domain comprising the amino acid sequence of SEQ ID NO: 17 and a VL domain comprising the amino acid sequence of SEQ ID NO: 19; and/or
(d) the anti-TIGIT antagonist antibody comprises a VH domain comprising the amino acid sequence of SEQ ID NO: 18 and a VL domain comprising the amino acid sequence of SEQ ID NO: 19.

515. The method of claim 504, wherein the PD-1 axis binding antagonist is selected from the group consisting of a PD-L1 binding antagonist, a PD-1 binding antagonist, and a PD-L2 binding antagonist.

516. The method of claim 515, wherein the PD-1 axis binding antagonist is an anti-PD-L1 antagonist antibody.

517. The method of claim 516, wherein:
(a) the anti-PD-L1 antagonist antibody is atezolizumab, MDX-1105, durvalumab, avelumab, SHR-1316, CS1001, envafolimab, TQB2450, ZKAB001, LP-002, CX-072, IMC-001, KL-A167, APL-502, cosibelimab, lodapolimab, FAZ053, TG-1501, BGB-A333, BCD-135, AK-106, LDP, GR1405, HLX20, MSB2311, RC98, PDL-GEX, KD036, KY1003, YBL-007, or HS-636;
(b) the anti-PD-L1 antagonist antibody comprises the following HVRs:
an HVR-H1 sequence comprising the amino acid sequence of GFTFSDSWIH (SEQ ID NO: 20);
an HVR-H2 sequence comprising the amino acid sequence of AWISPYGGSTYYADSVKG (SEQ ID NO: 21);
an HVR-H3 sequence comprising the amino acid sequence of RHWPGGFDY (SEQ ID NO: 22);
an HVR-L1 sequence comprising the amino acid sequence of RASQDVSTAVA (SEQ ID NO: 23);
an HVR-L2 sequence comprising the amino acid sequence of SASFLYS (SEQ ID NO: 24); and
an HVR-L3 sequence comprising the amino acid sequence of QQYLYHPAT (SEQ ID NO: 25);
and/or
(c) the anti-PD-L1 antagonist antibody comprises a VH domain comprising the amino acid sequence of SEQ ID NO: 26 and a VL domain comprising the amino acid sequence of SEQ ID NO: 27.

518. The method of claim 516, wherein the anti-PD-L1 antagonist antibody is a monoclonal antibody and/or a humanized antibody.

519. The method of claim 516, wherein the anti-PD-L1 antagonist antibody is (a) a full-length antibody or (b) an antibody fragment that binds PD-L1 selected from the group consisting of Fab, Fab′, Fab′-SH, Fv, scFv, and (Fab′)₂ fragments.

520. The method of claim 515, wherein the PD-1 axis binding antagonist is an anti-PD-1 antagonist antibody.

521. The method of claim 504, wherein the method comprises:
(a) administering to the subject or population of subjects the anti-TIGIT antagonist antibody at a dose of (i) between about 500 mg to about 700 mg every three weeks or (ii) about 600 mg every three weeks and/or administering to the subject or population of subjects the PD-1 axis binding antagonist at a dose of (i) between about 900 mg to about 1500 mg every three weeks or (ii) about 1200 mg every three weeks;
(b) administering to the subject or population of subjects the anti-TIGIT antagonist antibody at a dose of (i) about 700 mg to about 1000 mg every four weeks or (ii) about 840 mg every four weeks and/or administering to the subject or population of subjects the PD-1 axis binding antagonist at a dose of (i) about 1400 mg to 2000 mg every four weeks or (ii) about 1680 mg every four weeks; or
(c) administering to the subject or population of subjects the anti-TIGIT antagonist antibody at a dose of (i) about 300 mg to about 600 mg every two weeks or (ii) about 420 mg every two weeks and/or administering to the subject or population of subjects the PD-1 axis binding antagonist at a dose of (i) between about 600 mg to about 1200 mg every two weeks or (ii) about 840 mg every two weeks.

522. The method of claim 504, wherein the method comprises (a) administering to the subject or population of subjects the PD-1 axis binding antagonist before the anti-TIGIT antagonist antibody; (b) administering to the subject or population of subjects the anti-TIGIT antagonist antibody before the PD-1 axis binding antagonist; or (c) administering to the subject or population of subjects the PD-1 axis binding antagonist simultaneously with the anti-TIGIT antagonist antibody.

523. A method of treating a subject or population of subjects having a lung cancer, the method comprising administering to the subject or population of subjects a dosing regimen comprising one or more dosing cycles of an anti-TIGIT antagonist antibody, a PD-1 axis binding antagonist, a first chemotherapeutic agent which is a platinum-based chemotherapeutic agent, and a second chemotherapeutic agent which is a non-platinum-based chemotherapeutic agent.

524. The method of claim 523, wherein the subject or subjects do not have an epidermal growth factor receptor (EGFR) or anaplastic lymphoma kinase (ALK) genomic tumor aberration and/or the subject or subjects have received no prior systemic therapy for the lung cancer.

525. The method of claim 523, wherein the lung cancer is an NSCLC, in particular a locally advanced unresectable or metastatic non-squamous NSCLC.

526. The method of claim 523, wherein the dosing regimen comprises an induction phase comprising four dosing cycles, and wherein the anti-TIGIT antagonist antibody, the PD-1 axis binding antagonist, the platinum-based chemotherapeutic agent, and the non-platinum-based chemotherapeutic agent are administered on Day 1 of each dosing cycle of the induction phase.

527. The method of claim 526, wherein the dosing regimen comprises a maintenance phase following the induction phase, wherein the maintenance phase comprises one or more dosing cycles, and wherein the anti-TIGIT antagonist antibody, the PD-1 axis binding antagonist, and the non-platinum-based chemotherapeutic agent are administered on Day 1 of each dosing cycle of the maintenance phase.

528. The method of claim 527, wherein the one or more dosing cycles of the maintenance phase do not comprise administration of the platinum-based chemotherapeutic agent.

529. The method of claim 523, wherein the (a) platinum-based chemotherapeutic agent is carboplatin or cisplatin and the non-platinum-based chemotherapeutic agent is pemetrexed, (b) the anti-TIGIT antagonist antibody is tiragolumab, and/or (c) the PD-1 axis binding antagonist is atezolizumab.

530. A method for treating a subject having a lung cancer, the method comprising administering to the subject one or more dosing cycles of an anti-TIGIT antagonist antibody at a dose of between about 500 mg to about 700 mg every three weeks and a PD-1 axis binding antagonist at a dose of between about 900 mg to about 1500 mg every three weeks; wherein:
(a) the lung cancer is a resectable lung cancer; and/or
(b) at least one of the dosing cycles comprises administering to the subject the anti-TIGIT antagonist antibody and the PD-1 axis binding antagonist as a neoadjuvant treatment.

531. The method of claim 530, wherein the lung cancer is a resectable lung cancer; an early-stage lung cancer; and/or a stage II, IIIA, or IIIB lung cancer.

532. The method of claim 530, wherein (a) the first dosing cycle is initiated prior to a surgery or (b) at least one dosing cycle is initiated after a surgery.

533. The method of claim 530, wherein the method further comprises a radiotherapy.

534. The method of claim 533, wherein the radiotherapy is a post-operative radiotherapy.

535. The method of claim 530, wherein the method further comprises administering one or more chemotherapeutic agents.

536. The method of claim 535, wherein the one or more chemotherapeutic agents are administered after a surgery.

537. The method of claim 536, wherein the one or more chemotherapeutic agents are administered in 4 dosing cycles after the surgery.

538. The method of claim 535, wherein the one or more chemotherapeutic agents are a platinum-based chemotherapeutic agent and a non-platinum-based chemotherapeutic agent.

539. The method of claim 538, wherein:
(a) the platinum-based chemotherapeutic agent is carboplatin and the non-platinum-based chemotherapeutic agent is pemetrexed;
(b) the platinum-based chemotherapeutic agent is carboplatin and the non-platinum-based chemotherapeutic agent is gemcitabine;
(c) the platinum-based chemotherapeutic agent is carboplatin and the non-platinum-based chemotherapeutic agent is paclitaxel;
(d) the platinum-based chemotherapeutic agent is cisplatin and the non-platinum-based chemotherapeutic agent is pemetrexed; or
(e) the platinum-based chemotherapeutic agent is cisplatin and the non-platinum-based chemotherapeutic agent is gemcitabine;
(f) the anti-TIGIT antagonist antibody is tiragolumab; and/or
(g) the PD-1 axis binding antagonist is atezolizumab.

540. The method of claim 530, wherein the treating results in (a) an increase in major pathological response (MPR) rate as compared to a reference MPR rate; (b) a pathological complete response (pCR) and/or an increase in pCR rate as compared to a reference pCR rate; and/or (c) an increase in event-free survival (EFS) as compared to a reference EFS time.

541. The method of claim 530, wherein the lung cancer is an NSCLC.

542. A method for treating a subject or population of subjects having a cervical cancer with a detectable expression level of PD-L1, the method comprising administering to the subject or population of subjects one or more dosing cycles of an anti-TIGIT antagonist antibody at a dose of between about 500 mg to about 700 mg every three weeks and a PD-1 axis binding antagonist at a dose of between about 900 mg to about 1500 mg every three weeks.

543. The method of claim 542, wherein the cervical cancer is a Stage IVB, metastatic, recurrent, or persistent squamous cell carcinoma, adenosquamous carcinoma, or adenocarcinoma.

544. The method of claim 542, wherein (a) the subject or subjects have received at least one line of prior therapy or (b) the subject or subjects have not received prior therapy.

545. The method of claim 544, wherein the prior therapy is chemotherapy, surgery, and/or radiotherapy.

546. The method of claim 542, wherein the treating results in a clinical response.

547. The method of claim 542, wherein the anti-TIGIT antagonist antibody is tiragolumab and/or the PD-1 axis binding antagonist is atezolizumab.

548. A method of treating a subject or population of subjects having a breast cancer, the method comprising administering to the subject or population of subjects a dosing regimen comprising one or more dosing cycles of an anti-TIGIT antagonist antibody at a dose of about 700 mg to about 1000 mg every four weeks, a PD-1 axis binding antagonist at a dose of about 1400 mg to 2000 mg every four weeks, and a taxane at a dose of about 50 mg/m² to about 200 mg/m² for 3-weeks on/1-week off.

549. The method of claim 548, wherein the breast cancer is an unresectable locally advanced or metastatic triple-negative breast cancer (TNBC).

550. The method of claim 548, wherein the subject or subjects have not received prior systemic therapy for metastatic breast cancer.

551. A method of treating a subject having an early triple-negative breast cancer (eTNBC), the method comprising administering to the subject a dosing regimen comprising one or more dosing cycles of an anti-TIGIT antagonist antibody at a dose of about 300 mg to about 600 mg every two weeks and a PD-1 axis binding antagonist at a dose of about 600 mg to about 1200 mg every two weeks.

552. The method of claim 551, further comprising administering to the subject:
(a) one or more chemotherapeutic agents; or
(b) (i) one or more dosing cycles of the anti-TIGIT antagonist antibody, the PD-1 axis binding antagonist, and a taxane or a taxane and a platinum-based chemotherapeutic agent; and (ii) one or more dosing cycles of the anti-TIGIT antagonist antibody, the PD-1 axis binding antagonist, a topoisomerase II inhibitor, an alkylating agent, and granulocyte colony-stimulating factor (G-CSF) or granulocyte-macrophage colony-stimulating factor (GM-CSF).

553. The method of claim 552, wherein the one or more chemotherapeutic agents are a platinum-based chemotherapeutic agent, a taxane, a topoisomerase II inhibitor, or an alkylating agent.

554. The method of claim 553, wherein the alkylating agent is cyclophosphamide or the G-CSF is pegfilgrastim or filgrastim.

555. The method of claim 554, wherein the cyclophosphamide is administered at a dose of about 600 mg/m² or the pegfilgrastim is administered at a dose of about 6 mg.

556. The method of claim 552, wherein the method comprises further administering to the subject one or more subsequent doses of the one or more chemotherapeutic agents and/or G-CSF or GM-CSF.

557. The method of claim 556, wherein:
(a) the one or more chemotherapeutic agents and/or G-CSF or GM-CSF are each administered once per week, once every two weeks, or once every three weeks; or
(b) the platinum-based chemotherapeutic agent is administered every three weeks, the taxane is administered every week, the topoisomerase II inhibitor is administered every two weeks, the alkylating agent is administered every two weeks, and the G-CSF or GM-CSF is administered every two weeks.

558. The method of claim 553, wherein the taxane or the taxane and the platinum-based chemotherapeutic agent are administered for the first 12 weeks of the dosing regimen.

559. The method of claim 553, wherein the topoisomerase II inhibitor, the alkylating agent, and the G-CSF or GM-CSF are administered during weeks 13-19 of the dosing regimen.

560. The method of claim 551, wherein the method is a neoadjuvant treatment.

561. The method of claim 551, wherein the dosing regimen is followed by surgery.

562. The method of claim 553, wherein the topoisomerase II inhibitor is doxorubicin; the taxane is nab-paclitaxel and the platinum-based chemotherapeutic agent is carboplatin; the anti-TIGIT antagonist antibody is tiragolumab; and/or the PD-1 axis binding antagonist is atezolizumab.

563. The method of claim 551, wherein the treatment results in a pathological complete response (pCR) or an increase in overall survival (OS) or event-free survival (EFS).

564. A method for treating a subject or population of subjects having an SCCHN with a detectable expression level of PD-L1, the method comprising administering to the subject or population of subjects one or more dosing cycles of an anti-TIGIT antagonist antibody at a dose of between about 500 mg to about 700 mg every three weeks and a PD-1 axis binding antagonist at a dose of between about 900 mg to about 1500 mg every three weeks.

565. The method of claim 564, wherein (a) a tumor sample obtained from the subject or subjects have been determined to have a detectable expression level of PD-L1 and/or (b) the detectable expression level of PD-L1 is a detectable protein expression level of PD-L1 determined by an immunohistochemical (IHC) assay comprising staining with anti-PD-L1 antibody SP263.

566. The method of claim 564, wherein the SCCHN is a recurrent and/or metastatic SCCHN.

567. The method of claim 564, wherein the subject or subjects have not received prior therapy.

568. The method of claim 564, wherein the treating results in a CR or PR; an increase in the objective response rate (ORR) of the population of subjects as compared to a reference ORR; an increase in the progression-free survival (PFS) of the subject or population of subjects as compared to a reference PFS time, an increase in the duration of response (DOR) of the subject or population of subjects as compared to a reference DOR time, or an increase in the overall survival (OS) of the subject or population of subjects as compared to a reference OS time.

569. The method of claim 564, wherein the anti-TIGIT antagonist antibody is tiragolumab and/or the PD-1 axis binding antagonist is atezolizumab.

570. A method of treating a subject or population of subjects having a hepatocellular carcinoma (HCC), the method comprising administering to the subject or population of subjects:
(a) one or more dosing cycles of an anti-TIGIT antagonist antibody and a PD-1 axis binding antagonist, wherein the subject or population of subjects have received no prior systemic treatment for HCC; or
(b) one or more dosing cycles of an anti-TIGIT antagonist antibody, a PD-1 axis binding antagonist, and a VEGF antagonist.

571. The method of claim 570, wherein:
(a) the VEGF antagonist is administered at a dose of about 5 mg/kg to about 25 mg/kg every three weeks; the anti-TIGIT antagonist antibody is administered at a dose of about 500 mg to about 700 mg every three weeks; and/or the PD-1 axis binding antagonist is administered at a dose of about 900 mg to about 1500 mg every three weeks;
(b) (i) the VEGF antagonist is administered at a dose of about 1 mg/kg to about 20 mg/kg every two weeks; about 5 mg/kg to about 10 mg/kg every two weeks; or about 5 mg/kg, about 7.5 mg/kg, or about 10 mg/kg every two weeks; (ii) the anti-TIGIT antagonist antibody is administered at a dose of about 300 mg to about 800 mg every two weeks; and/or (iii) the PD-1 axis binding antagonist is administered at a dose of about 200 mg to about 1200 mg every two weeks; or
(c) the anti-TIGIT antagonist antibody is administered at a dose of about 700 mg to about 1000 mg every four weeks and/or the PD-1 axis binding antagonist is administered at a dose of about 1400 mg to about 2000 mg every four weeks.

572. The method of claim 570, wherein the HCC is a locally advanced or metastatic HCC and/or an unresectable HCC.

573. The method of claim 570, wherein the method comprises administering to the subject or population of subjects at least four dosing cycles.

574. The method of claim 570, wherein the subject or subjects have received no prior systemic treatment for HCC.

575. The method of claim 570, wherein the anti-TIGIT antagonist antibody is tiragolumab; the PD-1 axis binding antagonist is atezolizumab; and/or the VEGF antagonist is bevacizumab.

576. A method for treating a subject or population of subjects having an MIBC, the method comprising administering to the subject one or more dosing cycles of an anti-TIGIT antagonist antibody at a dose of between about 500 mg to about 700 mg every three weeks and a PD-1 axis binding antagonist at a dose of between about 900 mg to about 1500 mg every three weeks, wherein the subject is ineligible for treatment with a platinum-based chemotherapeutic agent and/or the treatment is a perioperative treatment.

577. The method of claim 576, wherein the platinum-based chemotherapeutic agent is cisplatin.

578. The method of claim 576, wherein the MIBC is surgically operable.

579. The method of claim 578, wherein the method further comprises a surgery.

580. The method of claim 579, wherein (a) at least one dosing cycle is initiated prior to the surgery and/or (b) at least one dosing cycle is initiated between 4-6 weeks after the surgery.

581. The method of claim 576, wherein the treating results in a pathological complete response (pCR); an increase in the recurrence-free survival (RFS) of the subject or subjects as compared to a reference RFS time; an increase in the event-free survival (EFS) of the subject or subjects as compared to a reference EFS time; an increase in the overall survival (OS) of the subject or subjects as compared to a reference OS time; or a pathological downstaging.

582. The method of claim 576, wherein the anti-TIGIT antagonist antibody is tiragolumab and/or the PD-1 axis binding antagonist is atezolizumab.

583. A method for treating a subject or population of subjects having an mUC, the method comprising administering to the subject or population of subjects a dosing regimen comprising one or more dosing cycles of an anti-TIGIT antagonist antibody at a dose of between about 500 mg to about 700 mg every three weeks and a PD-1 axis binding antagonist at a dose of between about 900 mg to about 1500 mg every three weeks.

584. The method of claim 583, wherein (a) the subject or subjects have not received a prior cancer immunotherapy or (b) the treatment is a second-line treatment.

585. The method of claim 583, wherein the mUC has progressed during or following a platinum-containing therapy.

586. The method of claim 583, wherein the method further comprises administering to the subject or population of subjects a second dosing regimen after the subject or population of subjects have experienced disease progression or unacceptable toxicity.

587. The method of claim 586, wherein the second dosing regimen comprises one or more dosing cycles of a PD-1 axis binding antagonist and an antibody-drug conjugate (ADC).

588. The method of claim 587, wherein the ADC is (a) enfortumab vedotin or (b) sacituzumab govitecan.

589. The method of claim 588, wherein (a) enfortumab vedotin is administered at a dose of 1.25 mg/kg every week for 2-weeks on/1 week off or (b) sacituzumab govitecan is administered at a dose of 10 mg/kg every week for 2-weeks on/1 week off.

590. The method of claim 583, wherein the treatment results in an ORR of the population of subjects of at least about 13.4% to at least about 31% and/or a median OS of the population of subjects of about 7.9 months to about 16.3 months.

591. The method of claim 583, wherein the anti-TIGIT antagonist antibody is tiragolumab and/or the PD-1 axis binding antagonist is atezolizumab.

592. A method of treating a subject or population of subjects having a pancreatic cancer, the method comprising administering to the subject or population of subjects a dosing regimen comprising one or more 28-day dosing cycles of an anti-TIGIT antagonist antibody at a dose of about 300 mg to about 600 mg every two weeks, a PD-1 axis binding antagonist at a dose of about 600 mg to about 1200 mg every two weeks, gemcitabine at a dose of about 1000 mg/m² three times every four weeks, and nab-paclitaxel at a dose of about 125 mg/m² three times every four weeks.

593. The method of claim 592, wherein the pancreatic cancer is a metastatic pancreatic ductal adenocarcinoma (PDAC).

594. The method of claim 593, wherein the subject or subjects have not received prior systemic therapy for metastatic PDAC.

595. The method of claim 592, wherein the treatment results in an ORR of the population of subjects of at least about 41.7% to about 46.7%; an increase in ORR of at least about 20% compared to a treatment comprising gemcitabine and nab-paclitaxel without an anti-TIGIT antagonist antibody and a PD-1 axis binding antagonist; a median PFS of the population of subjects of at least about 5.5 months to about 7 months; and/or a median OS of the population of subjects of at least about 8.5 months to about 10.6 months.

596. The method of claim 592, wherein:
(a) the anti-TIGIT antagonist antibody is tiragolumab, and the tiragolumab is administered at a dose of about 420 mg on Days 1 and 15 of each 28-day dosing cycle;
(b) the PD-1 axis binding antagonist is atezolizumab, and the atezolizumab is administered at a dose of about 840 mg on Days 1 and 15 of each 28-day dosing cycle;
(c) the gemcitabine is administered at a dose of about 1000 mg/m²; and
(d) the nab-paclitaxel is administered at a dose of about 125 mg/m².

597. A method for treating a subject or population of subjects having an esophageal cancer, the method comprising administering to the subject or population of subjects a dosing regimen comprising one or more 21-day dosing cycles of an anti-TIGIT antagonist antibody at a dose of between about 500 mg to about 700 mg on Day 1 of each dosing cycle and a PD-1 axis binding antagonist at a dose of between about 900 mg to about 1500 mg on Day 1 of each dosing cycle, wherein (a) the esophageal cancer is an advanced or metastatic esophageal cancer and/or (b) the subject or subjects have been previously treated with a platinum-based chemotherapeutic agent and a non-platinum-based chemotherapeutic agent.

598. The method of claim 597, wherein the subject or subjects have experienced disease progression or unacceptable toxicity during the previous treatment.

599. The method of claim 597, wherein the 21-day dosing cycles further comprise a platinum-based chemotherapeutic agent and a non-platinum-based chemotherapeutic agent.

600. The method of claim 599, wherein the platinum-based chemotherapeutic agent is omitted from the dosing regimen after six doses.

601. The method of claim 597, wherein (a) the platinum-based chemotherapeutic agent is cisplatin; (b) the non-platinum-based chemotherapeutic agent is an antimetabolite; (c) the anti-TIGIT antagonist antibody is tiragolumab; and/or (d) the PD-1 axis binding antagonist is atezolizumab.

602. The method of claim 601, wherein cisplatin is administered at a dose of about 80 mg/m² on Day 1 of each dosing cycle.

603. The method of claim 601, wherein the antimetabolite is 5-fluorouracil.

604. The method of claim 603, wherein 5-fluorouracil is administered at a dose of 800 mg/m²/24 hours on Days 1-5 of each 21-day cycle.

605. The method of claim 597, wherein the esophageal cancer is an advanced or metastatic esophageal cancer.

606. The method of claim 597, wherein the subject or subjects have had no prior treatment for metastatic esophageal cancer.

607. A method of treating a subject having a cancer, the method comprising administering to the subject a dosing regimen comprising:
one or more dosing cycles of an anti-TIGIT antagonist antibody at a dose of about 500 mg to about 700 mg every three weeks, a PD-1 axis binding antagonist at a dose of about 900 mg to about 1500 mg every three weeks, and:
(a) a platinum-based chemotherapeutic agent every three weeks, and a non-platinum-based chemotherapeutic agent every three weeks;
(b) an antimetabolite at a dose of between about 10 mg/m² to about 10000 mg/m² twice a day orally every three weeks for 2-weeks on/1-week off;
(c) gemcitabine and nab-paclitaxel; or
(d) a VEGF antagonist at a dose of between about 1 mg/kg to about 35 mg/kg every three weeks.

608. The method of claim 607, wherein the non-platinum-based chemotherapeutic agent is a topoisomerase II inhibitor.

609. The method of claim 607, wherein the topoisomerase II inhibitor is etoposide, teniposide, doxorubicin, daunorubicin, mitoxantrone, amsacrine, an ellipticine, aurintricarboxylic acid, or HU-331, in particular etoposide.

610. The method of claim 607, wherein the platinum-based chemotherapeutic agent is carboplatin or cisplatin, in particular carboplatin; the anti-TIGIT antagonist antibody is tiragolumab; and/or the PD-1 axis binding antagonist is atezolizumab.

611. The method of claim 607, wherein the VEGF antagonist is an anti-VEGF antibody.

612. The method of claim 611, wherein the anti-VEGF antibody is bevacizumab.

613. The method of claim 607, wherein the cancer is lung cancer; a small cell lung cancer (SCLC), in particular extensive stage SCLC (ES-SCLC); a non-small cell lung cancer (NSCLC), in particular locally advanced unresectable NSCLC (Stage IIIB NSCLC); locally advanced unresectable or metastatic non-squamous NSCLC; resectable lung cancer; cervical cancer; an early triple-negative breast cancer (eTNBC); a head and neck cancer; a liver cancer, in particular hepatocellular carcinoma (HCC); a bladder cancer; a urothelial carcinoma (UC), in particular a metastatic urothelial carcinoma (mUC); a pancreatic cancer, in particular a pancreatic ductal adenocarcinoma (PDAC); or an esophageal cancer, in particular an advanced or metastatic esophageal cancer.

614. A method of treating a subject having a cancer, the method comprising administering to the subject a dosing regimen comprising one or more dosing cycles of an anti-TIGIT antagonist antibody at a dose of about 700 mg to about 1000 mg every four weeks and a PD-1 axis binding antagonist at a dose of about 1400 mg to 2000 mg every four weeks.

615. The method of claim 614, wherein the cancer is lung cancer; a small cell lung cancer (SCLC), in particular extensive stage SCLC (ES-SCLC); a non-small cell lung cancer (NSCLC), in particular locally advanced unresectable NSCLC (Stage IIIB NSCLC); locally advanced unresectable or metastatic non-squamous NSCLC; resectable lung cancer; cervical cancer; an early triple-negative breast cancer (eTNBC); a head and neck cancer; a liver cancer, in particular hepatocellular carcinoma (HCC); a bladder cancer; a urothelial carcinoma (UC), in particular a metastatic urothelial carcinoma (mUC); a pancreatic cancer, in particular a pancreatic ductal adenocarcinoma (PDAC); or an esophageal cancer, in particular an advanced or metastatic esophageal cancer.

616. The method of claim 614, wherein the anti-TIGIT antagonist antibody is tiragolumab and/or the PD-1 axis binding antagonist is atezolizumab.

617. A method of treating a subject having a cancer, the method comprising administering to the subject a dosing regimen comprising one or more dosing cycles of an anti-TIGIT antagonist antibody at a dose of about 300 mg to about 600 mg every two weeks and a PD-1 axis binding antagonist at a dose of about 600 mg to about 1200 mg every two weeks.

618. The method of claim 617, wherein the cancer is lung cancer; a small cell lung cancer (SCLC), in particular extensive stage SCLC (ES-SCLC); a non-small cell lung cancer (NSCLC), in particular locally advanced unresectable NSCLC (Stage IIIB NSCLC); locally advanced unresectable or metastatic non-squamous NSCLC; resectable lung cancer; cervical cancer; an early triple-negative breast cancer (eTNBC); a head and neck cancer; a liver cancer, in particular hepatocellular carcinoma (HCC); a bladder cancer; a urothelial carcinoma (UC), in particular a metastatic urothelial carcinoma (mUC); a pancreatic cancer, in particular a pancreatic ductal adenocarcinoma (PDAC); or an esophageal cancer, in particular an advanced or metastatic esophageal cancer.

619. The method of claim 617, wherein the anti-TIGIT antagonist antibody is tiragolumab and/or the PD-1 axis binding antagonist is atezolizumab.

620. A method of treating a subject having a cancer, the method comprising administering to the subject a dosing regimen comprising one or more dosing cycles of an anti-TIGIT antagonist antibody at a dose of about 500 mg to about 700 mg every three weeks and:
(a) pembrolizumab at a dose of about 100 mg to about 300 mg every three weeks; or
(b) pembrolizumab at a dose of between about 300 mg to about 500 mg every six weeks

621. The method of claim 620, wherein the cancer is a solid tumor and/or is locally advanced or metastatic.

622. The method of claim 620, wherein the cancer is a lung cancer, a pancreatic cancer, a cervical cancer, a breast cancer, a head and neck cancer, a liver cancer, a bladder cancer, an esophageal cancer, a gastric cancer, a colorectal cancer, a kidney cancer, a renal cancer, a melanoma, or an ovarian cancer.

623. The method of claim 620, wherein the anti-TIGIT antagonist antibody is tiragolumab.


SEQUENCE LISTING (34 sequences)

SEQ ID NO: 1 - protein, 7 aa, Artificial Sequence
  Synthetic Construct
SNSAAWN

SEQ ID NO: 2 - protein, 18 aa, Artificial Sequence
  Synthetic Construct
KTYYRFKWYSDYAVSVKG

SEQ ID NO: 3 - protein, 14 aa, Artificial Sequence
  Synthetic Construct
ESTTYDLLAGPFDY

SEQ ID NO: 4 - protein, 17 aa, Artificial Sequence
  Synthetic Construct
KSSQTVLYSSNNKKYLA

SEQ ID NO: 5 - protein, 7 aa, Artificial Sequence
  Synthetic Construct
WASTRES

SEQ ID NO: 6 - protein, 9 aa, Artificial Sequence
  Synthetic Construct
QQYYSTPFT

SEQ ID NO: 7 - protein, 23 aa, Artificial Sequence
  Synthetic Construct
DIVMTQSPDSLAVSLGERATINC

SEQ ID NO: 8 - protein, 15 aa, Artificial Sequence
  Synthetic Construct
WYQQKPGQPPNLLIY

SEQ ID NO: 9 - protein, 32 aa, Artificial Sequence
  Synthetic Construct
GVPDRFSGSGSGTDFTLTISSLQAEDVAVYYC

SEQ ID NO: 10 - protein, 10 aa, Artificial Sequence
  Synthetic Construct
FGPGTKVEIK

SEQ ID NO: 11 - protein, 30 aa, Artificial Sequence
  Synthetic Construct
  MISC_FEATURE (1)..(1): Xaa is Gln or Glu
XVQLQQSGPGLVKPSQTLSLTCAISGDSVS

SEQ ID NO: 12 - protein, 14 aa, Artificial Sequence
  Synthetic Construct
WIRQSPSRGLEWLG

SEQ ID NO: 13 - protein, 32 aa, Artificial Sequence
  Synthetic Construct
RITINPDTSKNQFSLQLNSVTPEDTAVFYCTR

SEQ ID NO: 14 - protein, 11 aa, Artificial Sequence
  Synthetic Construct
WGQGTLVTVSS

SEQ ID NO: 15 - protein, 30 aa, Artificial Sequence
  Synthetic Construct
EVQLQQSGPGLVKPSQTLSLTCAISGDSVS

SEQ ID NO: 16 - protein, 30 aa, Artificial Sequence
  Synthetic Construct
QVQLQQSGPGLVKPSQTLSLTCAISGDSVS

SEQ ID NO: 17 - protein, 126 aa, Artificial Sequence
  Synthetic Construct
EVQLQQSGPGLVKPSQTLSLTCAISGDSVSSNSAAWNWIRQSPSRGLEWLGKTYYRFKWYSDYAVSVKGRITINPDTSKNQFSLQLNSVTPEDTAVFYCTRESTTYDLLAGPFDYWGQGTLVTVSS

SEQ ID NO: 18 - protein, 126 aa, Artificial Sequence
  Synthetic Construct
QVQLQQSGPGLVKPSQTLSLTCAISGDSVSSNSAAWNWIRQSPSRGLEWLGKTYYRFKWYSDYAVSVKGRITINPDTSKNQFSLQLNSVTPEDTAVFYCTRESTTYDLLAGPFDYWGQGTLVTVSS

SEQ ID NO: 19 - protein, 113 aa, Artificial Sequence
  Synthetic Construct
DIVMTQSPDSLAVSLGERATINCKSSQTVLYSSNNKKYLAWYQQKPGQPPNLLIYWASTRESGVPDRFSGSGSGTDFTLTISSLQAEDVAVYYCQQYYSTPFTFGPGTKVEIK

SEQ ID NO: 20 - protein, 10 aa, Artificial Sequence
  Synthetic Construct
GFTFSDSWIH

SEQ ID NO: 21 - protein, 18 aa, Artificial Sequence
  Synthetic Construct
AWISPYGGSTYYADSVKG

SEQ ID NO: 22 - protein, 9 aa, Artificial Sequence
  Synthetic Construct
RHWPGGFDY

SEQ ID NO: 23 - protein, 11 aa, Artificial Sequence
  Synthetic Construct
RASQDVSTAVA

SEQ ID NO: 24 - protein, 7 aa, Artificial Sequence
  Synthetic Construct
SASFLYS

SEQ ID NO: 25 - protein, 9 aa, Artificial Sequence
  Synthetic Construct
QQYLYHPAT

SEQ ID NO: 26 - protein, 118 aa, Artificial Sequence [sequence omitted]

SEQ ID NO: 27 - protein, 108 aa, Artificial Sequence
  Synthetic Construct
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKR

SEQ ID NO: 28 - protein, 447 aa, Artificial Sequence
  Synthetic Construct
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 29 - protein, 214 aa, Artificial Sequence
  Synthetic Construct
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 30 - protein, 244 aa, Homo sapiens
MRWCLLLIWAQGLRQAPLASGMMTGTIETTGNISAEKGGSIILQCHLSSTTAQVTQVNWEQQDQLLAICNADLGWHISPSFKDRVAPGPGLGLTLQSLTVNDTGEYFCIYHTYPDGTYTGRIFLEVLESSVAEHGARFQIPLLGAMAATLVVICTAVIVVVALTRKKKALRIHSVEGDLRRKSAGQEEWSPSAPSPPGSCVQAEAAPAGLCGEQRGEDCAELHDYFNVLSYRSLGNCSFFTETG

SEQ ID NO: 31 - protein, 223 aa, Homo sapiens [sequence omitted]

SEQ ID NO: 32 - protein, 290 aa, Homo sapiens
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 33 - protein, 456 aa, Homo sapiens
EVQLQQSGPGLVKPSQTLSLTCAISGDSVSSNSAAWNWIRQSPSRGLEWLGKTYYRFKWYSDYAVSVKGRITINPDTSKNQFSLQLNSVTPEDTAVFYCTRESTTYDLLAGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 34 - protein, 220 aa, Homo sapiens
DIVMTQSPDSLAVSLGERATINCKSSQTVLYSSNNKKYLAWYQQKPGQPPNLLIYWASTRESGVPDRFSGSGSGTDFTLTISSLQAEDVAVYYCQQYYSTPFTFGPGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC